(19) United States
(12) Patent Application Publication
(10) Pub. No.: US 2026/0131007 A1
(43) Pub. Date: May 14, 2026

(54) Mannose 6-Phosphate Receptor Binding Compounds for the Degradation of Extracellular Proteins

(71) Applicant: Avilar Therapeutics, Inc., Waltham, MA (US)

(72) Inventors: Jason Allan Wiles, Madison, CT (US); Hu Liu, Newton, MA (US); Srinivasa Karra, Pembroke, MA (US); Philip B. Graham, Palm Springs, CA (US)

(73) Assignee: Avilar Therapeutics, Inc., Waltham, MA (US)

(21) Appl. No.: 19/316,915

(22) Filed: Sep. 2, 2025

Related U.S. Application Data
(63) Continuation of application No. PCT/US24/18217, filed on Mar. 1, 2024.
(60) Provisional application No. 63/449,542, filed on Mar. 2, 2023.

(51) Int. Cl. A61K 47/54

(52) U.S. Cl. CPC A61K 47/549

(57) ABSTRACT

Compounds and compositions that have a Mannose 6-Phosphate Receptor Ligand bound to an Extracellular Protein Binding Ligand are provided for the selective degradation of a target extracellular protein in vivo to treat disorders mediated by the extracellular protein.

### CROSS REFERENCE TO RELATED APPLICATION

This application is a continuation of International Patent Application No. PCT/US2024/18217, filed in the U.S. Receiving Office on Mar. 1, 2024, which claims the benefit of U.S. Provisional Application No. 63/449,542, filed Mar. 2, 2023. The entirety of each of these applications is hereby incorporated by reference for all purposes.

### FIELD OF THE INVENTION

This invention provides compounds and compositions that have a mannose 6-phosphate receptor binding ligand bound to an extracellular protein binding ligand for the selective degradation of a target extracellular protein in vivo to treat disorders mediated by the extracellular protein.

### INCORPORATION BY REFERENCE

The Sequence Listing XML file named “19121-029WO1US1_ST26_2” created on Jan. 27, 2026, and having a size of 687,401 bytes, is hereby incorporated by reference.

### BACKGROUND OF THE INVENTION

Historically, therapeutic strategies for the inhibition of proteins employed small molecule inhibitors which bound in an enzymatic pocket or at an allosteric position. Those proteins which were not enzymes were difficult to control, and some were considered “not druggable.”

Intracellular protein degradation is a natural and highly regulated, essential process that maintains cellular homeostasis. The selective identification and removal of damaged, misfolded, or excess proteins within the cell is achieved via the ubiquitin-proteasome pathway (UPP). The UPP is central to the regulation of almost all intracellular processes. A number of companies and institutions have designed intracellular protein degrading molecules that take advantage of this natural process to degrade disease-mediating proteins intracellularly by linking a ligand to the protein to be degraded to a protein in the UPP. Examples are found in U.S. 2014/0356322 assigned to Yale University, GlaxoSmithKline, and Cambridge Enterprise Limited University of Cambridge; Buckley et al. (J. Am. Chem. Soc. 2012, 134, 4465-4468) titled “Targeting the Von Hippel-Lindau E3 Ubiquitin Ligase Using Small Molecules to Disrupt the Vhl/Hif-1alpha Interaction”; WO 2015/160845 assigned to Arvinas Inc. titled “Imide Based Modulators of Proteolysis and Associated Methods of Use”; Lu et al. (Chem. Biol. 2015, 22, 755-763) titled “Hijacking the E3 Ubiquitin Ligase Cereblon to Efficiently Target Brd4”; Bondeson et al. (Nat. Chem. Biol. 2015, 11, 611-617) titled “Catalytic in Vivo Protein Knockdown by Small-Molecule Protacs”; Gustafson et al. (Angewandte Chemie, International Edition in English 2015, 54, 9659-9662) titled “Small-Molecule-Mediated Degradation of the Androgen Receptor through Hydrophobic Tagging”; Lai et al. (Angewandte Chemie, International Edition in English 2016, 55, 807-810) titled “Modular Protac Design for the Degradation of Oncogenic Bcr-Abl”; Toure et al. (Angew. Chem. Int. Ed. 2016, 55, 1966-1973) titled “Small-Molecule Protacs: New Approaches to Protein Degradation”; Winter et al. (Science 2015, 348, 1376-1381) titled “Drug Development. Phthalimide Conjugation as a Strategy for in Vivo Targeted Protein Degradation”; U.S. 2016/0058872 assigned to Arvinas, Inc. titled “Imide Based Modulators of Proteolysis and Associated Methods of Use” and U.S. 2016/0045607 assigned to Arvinas Inc. titled “Estrogen-related Receptor Alpha Based PROTAC Compounds and Associated Methods of Use”.

The highjacking of the UPP intracellular process to degrade difficult or undruggable proteins, however, is not available to degrade extracellular proteins. Nonlimiting examples of extracellular proteins that can play a strong role in creating or exacerbating serious diseases include immunoglobulins and cytokines, including IgA, IgG, IgD, IgE, and IgM.

The human body includes several cell surface receptors that function to regulate circulating extracellular protein concentration by transporting their extracellular protein target into the cell for degradation. Mannose 6-phosphate receptors (M6PR) and the asialoglycoprotein receptor (ASGPR) traffic proteins to the lysosome for degradation. There are two mannose 6-phosphate receptors, one of which is dependent on cation presence for efficient lysosomal activity (cation dependent mannose 6-phosphate receptor, also known as CD-M6PR and 46 kDa mannose 6-phosphate receptor) and another that does not depend on cation presence for efficient lysosomal activity (cation independent mannose 6-phosphate receptor, also known as CI-M6PR, IGF2 receptor, and CD222).

Preliminary research has been reported on the use of cell surface receptors for the targeted degradation of extracellular proteins. For example, WO2021/142377 assigned to Lycia Therapeutics describes compounds with ASGPR receptor ligands or mannose 6-phosphate receptor ligands for the degradation of extracellular proteins. WO2021/156792 assigned to Novartis AG describes additional compounds with ASGPR receptor ligands or mannose 6-phosphate and describes their use for the degradation of FHR³ and PCSK9.

The Board of Trustees of the Leland Stanford Junior University has filed a PCT application, WO2020/132100, which describes the use of compounds that bind a lysosomal targeting molecule such as the mannose 6-phophate receptor or ASGPR to degrade a cell surface molecule or extracellular molecule. Compounds related to the WO2020/132100 disclosure are described in an article by Banik et al. (“Lysosome-Targeting Chimaeras for Degradation of Extracellular Proteins” Nature, 2020, 584, 291). Additional research from Bertozzi, et al. was published in an article titled “Lysosome Targeting Chimeras (LYTACs) That Engage a Liver-Specific Asialoglycoprotein Receptor for Targeted Protein Degradation,” Nature 17, 937-946, (2021).

Lycia Therapeutics Inc. has filed applications including WO2021/142377, WO2021/263060 WO 2021/263061, WO 2022/150721, WO 2022/272307, WO 2022/271981, WO 2023/288033, and WO 2023/288015 which describe the use of ligands of a protein associated with endocytosis attached via a linker to an extracellular protein binding ligand.

Yale University and Biohaven Therapeutics Ltd. has filed applications including WO 2019/199621, WO 2019/199634, WO 2021/072269, WO 2021/072246, WO 2022/178428, WO 2022/178425, WO 2022/192478, and WO 2023/028597 which describe the use of certain cellular receptor targeting ligands covalently bound to a circulating protein binding moiety.

U.S. Pat. Nos. 9,340,553; 9,617,293; 10,039,778; 10,376,531, and 10,813,942 assigned to Pfizer Inc. describe certain bicyclic, bridged ketal derivatives of N-acetylgalactosamine (GalNAc) as targeting agents for the ASGPR receptor that in one embodiment are bound to a linker and/or a therapeutic agent.

Avilar Therapeutics Inc. describes new ASGPR binding ligands with various C2 substituents in WO2021/155317 and WO2022/235699. Avilar has described heterobifunctional compounds for targeted degradation with mannose 6-phosphate receptor ligands in WO 2023/028338. Avilar has also described other heterobifunctional compounds in WO 2023/009554 and WO 2022/035997.

While some progress has been made in the area of targeted degradation of disease-mediating extracellular proteins, much is left to be accomplished. There remains an unmet need for additional chemical compounds and approaches to treat medical disorders mediated by extracellular proteins.

### SUMMARY OF THE INVENTION

Novel compounds and their pharmaceutically acceptable salts and compositions thereof that degrade disease-mediating extracellular proteins, as well as starting materials and intermediates for such compounds and their methods of use and processes of manufacture are provided.

The extracellular protein degrading compounds described herein include new high affinity mannose 6-phosphate receptor ligands which have improved properties compared to mannose 6-phosphate for use in targeted extracellular protein degradation.

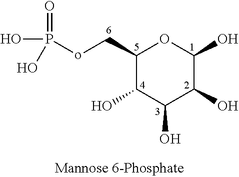

Extracellular proteins that can be targeted according to the present invention include but are not limited to immunoglobulins such as IgA, IgG, IgD, IgE, and IgM and cytokines such as interferons, interleukins, chemokines, lymphokines, MIP, growth factors, hormones, complement factors, neurotransmitters, capsids, soluble receptors, enzymes, and tumor necrosis factors. In certain embodiments, the extracellular protein is selected from IgA, IgG, IgE, IgM, TNF (α or β), IL-1b, IL-2, IFN-γ, IL-6, VEGF, TGF-b1, PCSK-9, Factor B, Factor D, Factor H and CC5. In other nonlimiting embodiments, a compound of the present invention crosses the blood brain barrier and can be used in the treatment of a CNS related disorder such as Alzheimer's disease.

In certain aspects, an Extracellular Protein Degrader of Formula I, Formula II, or Formula III is provided that includes a new Mannose 6-Phosphate Ligand:

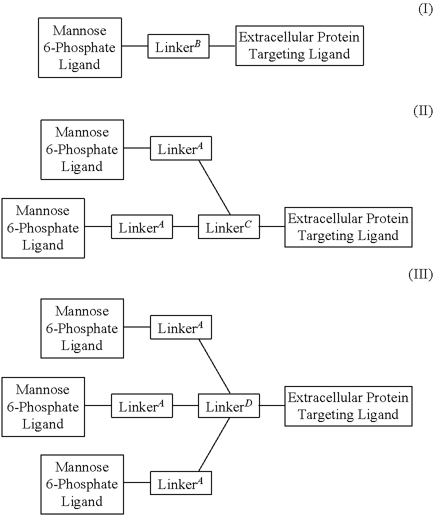

or a pharmaceutically acceptable salt thereof;
wherein:
Mannose 6-Phosphate Ligand is selected from:

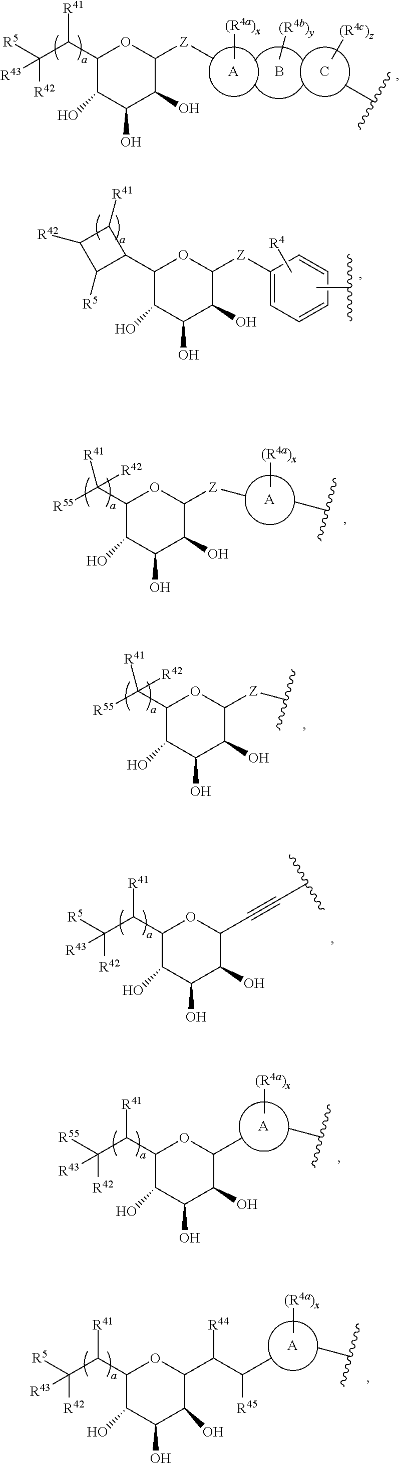

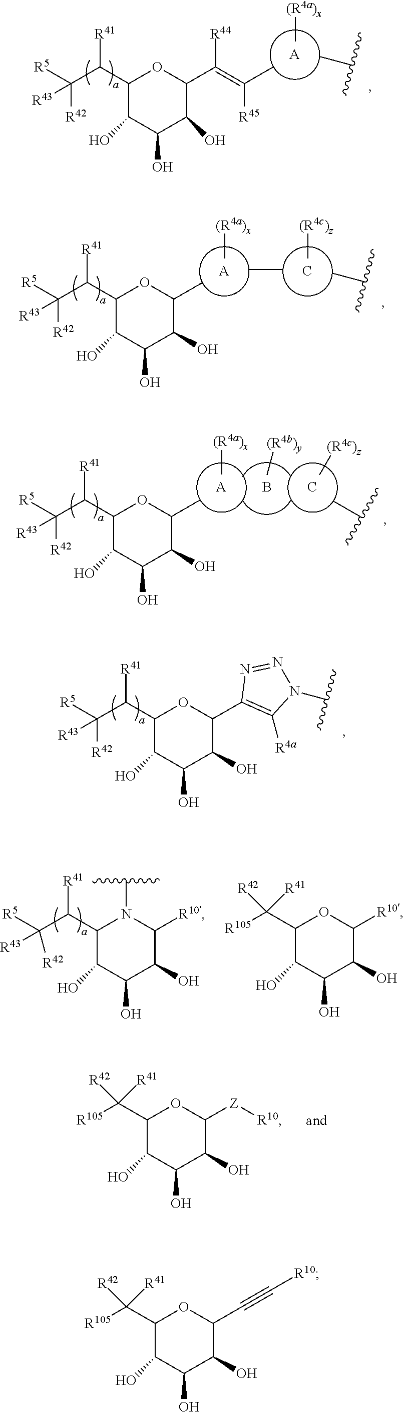

a, x, y, and z are independently 0, 1, 2, or 3;
Z is NR⁸, O, S, NC(O)R³, or CR⁴R⁸;
\textcircled{A} is cycloalkyl, heterocycle, aryl or heteroaryl;
\textcircled{B} is a nonaromatic cycloalkyl or heterocycle;
\textcircled{C} is aryl or heteroaryl;
for example, a compound of structure

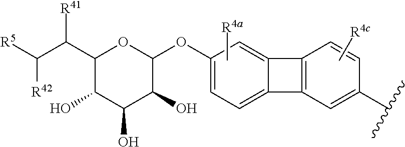

is a compound of the formula

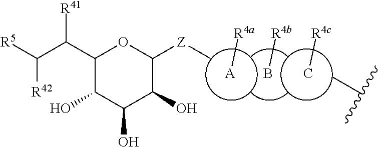

wherein \textcircled{A} is phenyl, \textcircled{B} is cyclobutyl, and \textcircled{C} is phenyl;
R³ at each occurrence is independently selected from hydrogen, alkyl, haloalkyl, arylalkyl, heteroarylalkyl, alkenyl, alkynyl, aryl, heteroaryl, heterocycle, —OR⁸, and —NR⁸R⁹;
R⁴, R^{4a}, R^{4b}, and R^{4c} are independently selected at each occurrence from hydrogen, F, Cl, Br, I, alkyl, haloalkyl, arylalkyl, heteroarylalkyl, alkenyl, alkynyl, aryl, heteroaryl, heterocycle, cyano, —OR⁶, —NR⁶R⁷, C(O)R³, S(O)R³, C(S)R³, and S(O)₂R³;
R⁴¹, R⁴², R⁴³, R⁴⁴, and R⁴⁵ are independently selected at each occurrence from hydrogen, alkyl, haloalkyl, F, arylalkyl, heteroarylalkyl, alkenyl, alkynyl, aryl, heteroaryl, and heterocycle;
R⁵⁰ is hydrogen, alkyl, haloalkyl, arylalkyl, heteroarylalkyl, alkenyl, alkynyl, aryl, heteroaryl, heterocycle, —OR⁶, or—NR⁶R⁷;
R⁵ is selected from

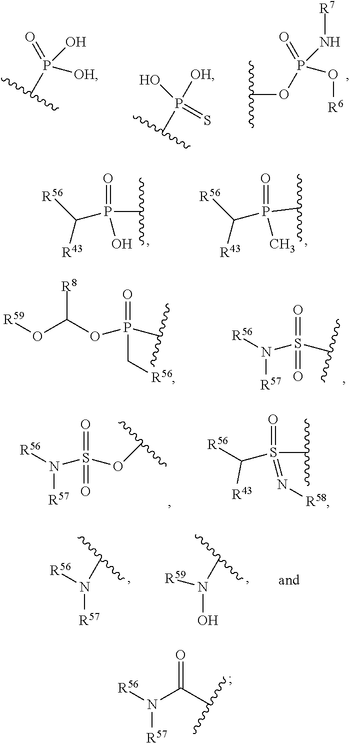

in other embodiments R⁵ is R⁵⁵;
R⁵⁵ is selected from

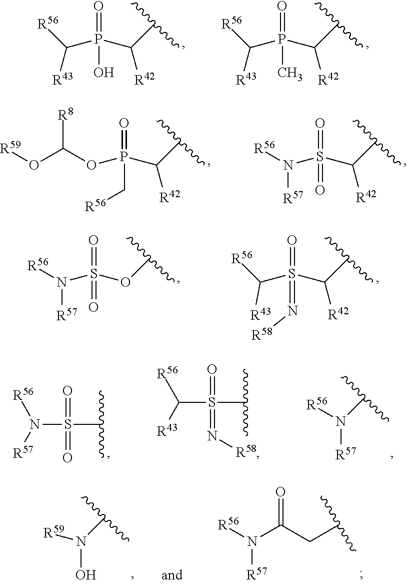

R⁵⁶ is independently selected from hydrogen, alkyl, alkenyl, alkynyl, haloalkyl, —O-alkyl, hydroxyl, cyano, heterocyclyl, aryl, and heteroaryl wherein each group except for hydrogen may optionally be substituted with 1, 2, or 3 independently selected R^{9a} substituents as allowed by valence;
R⁵⁷ is selected from hydrogen, alkyl, alkenyl, alkynyl, haloalkyl, —O-alkyl, aryl, heteroaryl, C(O) H, C(O) alkyl, C(O) alkenyl, C(O) alkynyl, C(O) haloalkyl, C(O) aryl, C(O)heteroaryl, C(O)O-alkyl, C(O)O-alkenyl, C(O)O-alkynyl, C(O)O-haloalkyl, C(O)O-aryl, C(O)O-heteroaryl and

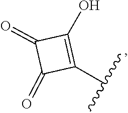 [SMILES: *c1c(O)c(=O)c1=O]

wherein each group except for hydrogen may optionally be substituted with 1, 2, or 3 independently selected R⁹% substituents as allowed by valence;
or R⁵⁶, R⁵⁷, and the nitrogen to which they are attached form a 5 to 7 membered heterocyclic ring such as but not limited to piperidine and piperazine which can be optionally substituted by alkyl, heterocycle, aryl, and heteroaryl, for example, a compound of the structure

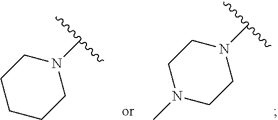

R⁵⁸ is selected from hydrogen, alkyl, C(O) alkyl, C(O)O-alkyl, C(O) aryl, C(O)heteroaryl, C(O)O-aryl, C(O)O-arylalkyl, and C(O)O-heteroaryl, wherein each group may optionally be substituted with 1, 2, or 3 independently selected R^{9c} substituents as allowed by valence;
R⁵⁹ is selected from C(O) alkyl, C(O)O-alkyl, C(O) aryl, C(O)heteroaryl, C(O)O-aryl, and C(O)O-heteroaryl, wherein each group may optionally be substituted with 1, 2, or 3 independently selected R^{9d} substituents as allowed by valence;
R⁶ and R⁷ are independently selected at each occurrence from hydrogen, alkyl, arylalkyl, heteroarylalkyl, alkenyl, alkynyl, aryl, haloalkyl, heteroaryl, heterocycle, -alkyl—OR⁸, -alkyl—NR⁸R⁹, C(O)R³, S(O)R³, C(S)R³, and S(O)₂R³;
R⁸ is independently selected at each occurrence from hydrogen, alkyl, arylalkyl, heteroarylalkyl, alkenyl, alkynyl, aryl, heteroaryl, and heterocycle;
R⁹ is independently selected at each occurrence from hydrogen, alkyl, arylalkyl, heteroarylalkyl, alkenyl, alkynyl, aryl, heteroaryl, and heterocycle;
R^{9a}, R^{9b}, R^{9c}, and R^{9d} are independently selected at each occurrence from hydrogen, F, Cl, Br, I, alkyl, haloalkyl, arylalkyl, heteroarylalkyl, alkenyl, alkynyl, aryl, heteroaryl, heterocycle, cyano, —OR⁶, —NR⁶R⁷, C(O)R³, S(O)R³, C(S)R³, and S(O)₂R³;
R¹⁰ is selected from hydrogen, alkyl, aryl, heteroaryl, arylalkyl, heteroarylalkyl, alkyl-O-alkyl, C(O) aryl, C(O) alkyl, C(O) arylalkyl, C(O)heteroarylalkyl, each of which R¹⁰ except hydrogen is optionally substituted with 1, 2, 3, or 4 substituents selected from alkyl, alkenyl, alkynyl, haloalkyl, —OR⁶, F, Cl, Br, I, —NR⁶R⁷, heterocycle, heteroaryl, aryl, cyano, nitro, hydroxyl, amide, —SR³, —S(O)(NR⁶)R³, —NR^{8c}(O)R³, —C(O)NR⁶R⁷, —C(O)OR³, and —C(O)R³;
or R¹⁰ is

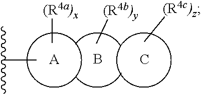

R¹⁰′ is selected from hydrogen, alkyl, aryl, heteroaryl, arylalkyl, heteroarylalkyl, each of which R¹⁰ except hydrogen is optionally substituted with 1, 2, 3, or 4 substituents selected from alkyl, alkenyl, alkynyl, haloalkyl, —OR⁶, F, Cl, Br, I, —NR⁶R⁷, heterocycle, heteroaryl, aryl, cyano, nitro, hydroxyl, amide, —SR³, —S(O)(NR⁶)R³, —NR⁸C(O)R³, —C(O)NR⁶R⁷, —C(O)OR³, and —C(O)R³;′
R¹⁰⁵ is selected from

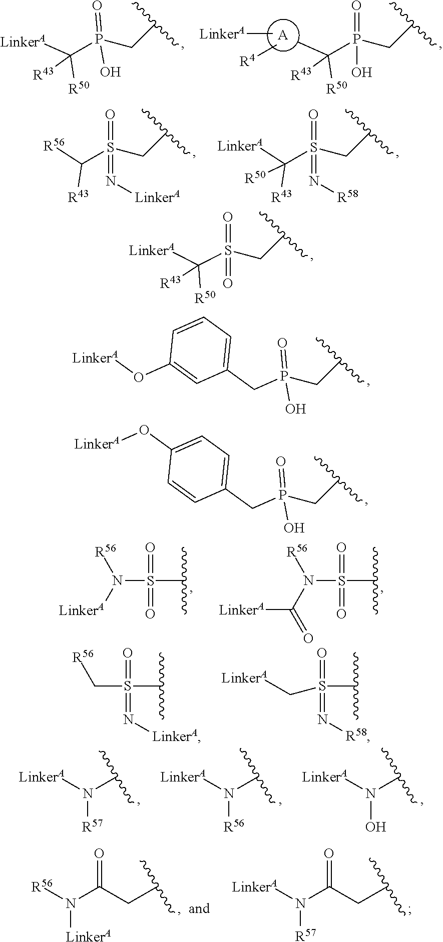

Linker^{A} and Linker^{B} are independently selected from:
wherein:

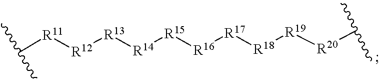

R¹¹, R¹², R¹³, R¹⁴, R¹⁵, R¹⁶, R¹⁷, R¹⁸, R¹⁹, and R²⁰ are independently at each occurrence selected from the group consisting of a bond, alkyl, —C(O)—, —C(O)O—, —OC(O)—, —SO₂—, —S(O)—, —C(S)—, —C(O)NR⁶—, —NR^{9c}(O)—, —C(O)N(OH)—, —N(OH)C(O)—, —O—, —S—, —NR⁶—, —C(R²¹R²¹)—, —S(O)(═N—R⁴⁴)—, —S(O)(═NR), —P(O)(R³)O—, —P(O)(R³)—, a divalent residue of a natural or unnatural amino acid, alkenyl, alkynyl, haloalkyl, alkoxy, aryl, heterocycle, heteroaryl, —CH₂CH₂—[O—(CH₂)₂]ₙ—O—, —CH₂CH₂—[O—(CH₂)₂]ₙ—NR⁶—, —CH₂CH₂—[O—(CH₂)₂]ₙ—, -[—(CH₂)₂—O-]ₙ-,
[O—(CH₂)₂]ₙ—, —[O—CH(CH₃)C(O)]ₙ—, —[C(O)—CH(CH₃)—O]ₙ-, —[O—CH₂C(O)]ₙ—, —[C(O)—CH₂—O]ₙ—, a divalent residue of a fatty acid, a divalent residue of an unsaturated or saturated mono- or dicarboxylic acid; each of which is optionally substituted with 1, 2, 3, or 4 substituents independently selected from R²¹, wherein Linker^{A} is attached to Linker^{B} and in certain embodiments Linker^{A} is a bond and denotes the attachment point to Linker^{B};
Linker^{C} is selected from:
wherein:

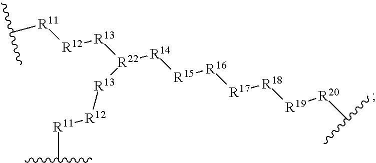

R²² is independently at each occurrence selected from the group consisting of alkyl, —C(O)N—, —NC(O)—, —N—, —C(R²¹)—, —P(O)O—, —P(O)—, —P(O)(NR⁶R⁷)N—, alkenyl, haloalkyl, aryl, heterocycle, and heteroaryl, each of which is optionally substituted with 1, 2, 3, or 4 substituents independently selected from R²¹;
Linker^{D} is selected from:

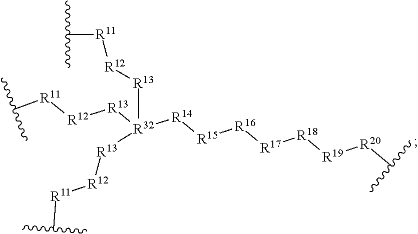

wherein:
R³² is independently at each occurrence selected from the group consisting of alkyl, N⁺X, —C—, alkenyl, haloalkyl, aryl, heterocycle, and heteroaryl, each of which is optionally substituted with 1, 2, 3, or 4 substituents independently selected from R²¹;
X⁻ is an anionic group, for example Br⁻ or Cl⁻;
n is independently selected at each instance from 0, 1, 2, 3, 4, 5, 6, 7, 8, 9, or 10;
R²¹ is independently at each occurrence selected from the group consisting of hydrogen, alkyl, alkenyl, alkynyl, F, Cl, Br, I, hydroxyl, alkoxy, azide, amino, cyano, —NR⁶R⁷, —NR⁸SO₂R³, —NR⁸S(O)R³, haloalkyl, aryl, heteroaryl, and heterocycle; and
Extracellular Protein Targeting Ligand is a chemical moiety that binds to the targeted disease-mediating extracellular protein.

In certain embodiments, the Extracellular Protein Targeting Ligand is a means for binding a targeted disease-mediating extracellular or membrane bound protein, as defined by 35 U.S.C. 112 (f).

In certain embodiments the Mannose 6-Phosphate Ligand is

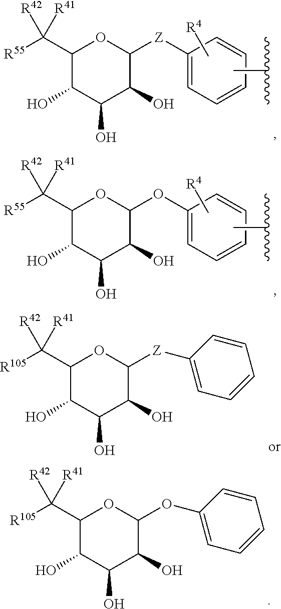

In certain embodiments the Mannose 6-Phosphate Ligand is

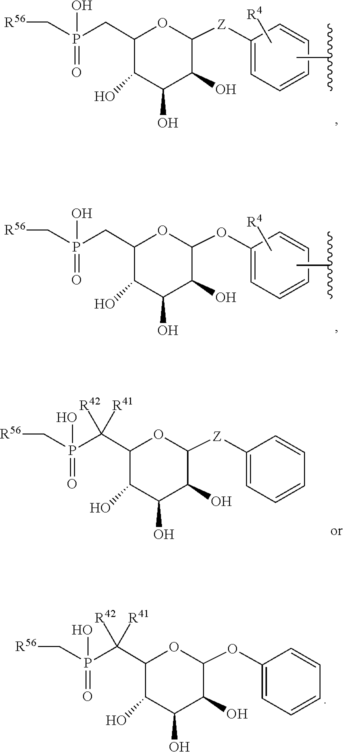

In another aspect of the invention the Mannose 6-Phosphate Ligand is

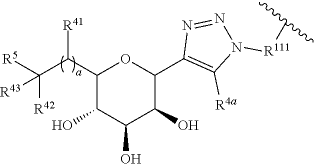

wherein R¹¹¹ is aryl, heteroaryl, heterocycle, C3-C10alkyl, alkenyl, or alkynyl each of which is optionally independently substituted by 1, 2; or 3 R^{4b} substituents and all other variables are as defined herein.

In certain embodiments R¹¹¹ is aryl optionally independently substituted by 1, 2; or 3 R^{4b} substituents and all other variables are as defined herein.

In certain embodiments R¹¹¹ is heteroaryl optionally independently substituted by 1, 2; or 3 R^{4b} substituents and all other variables are as defined herein.

In certain embodiments R¹¹¹ is C₃-C₁₀alkyl optionally independently substituted by 1, 2; or 3 R^{4b} substituents and all other variables are as defined herein.

In other aspects the Mannose 6-Phosphate Ligand is

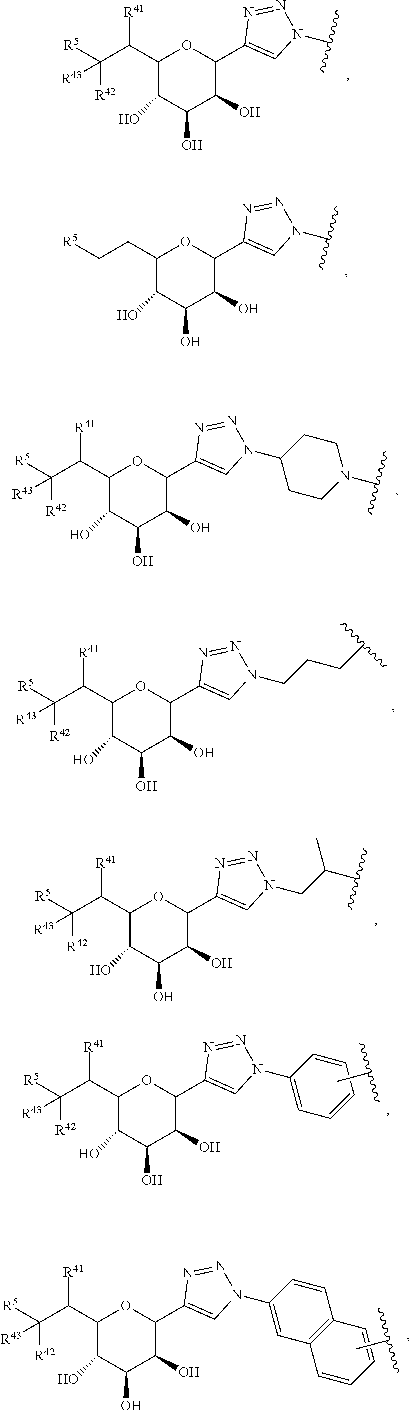

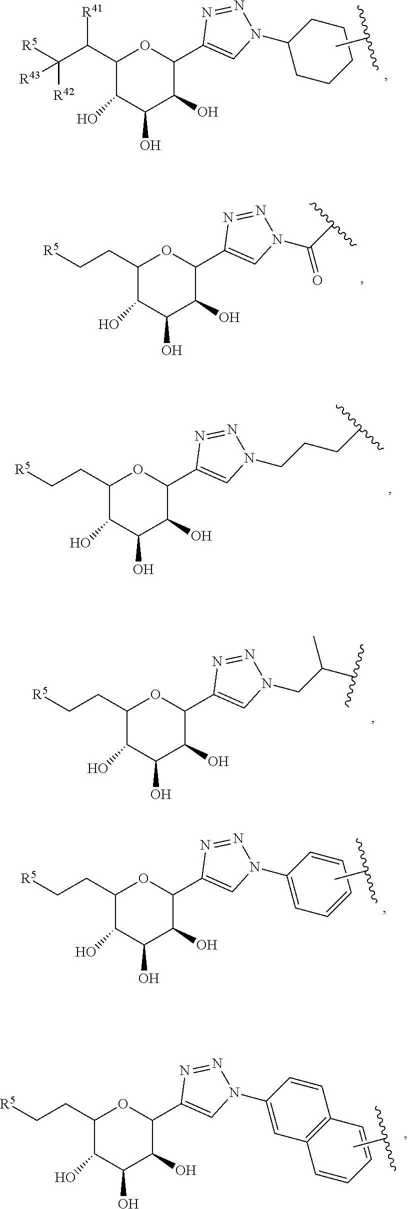

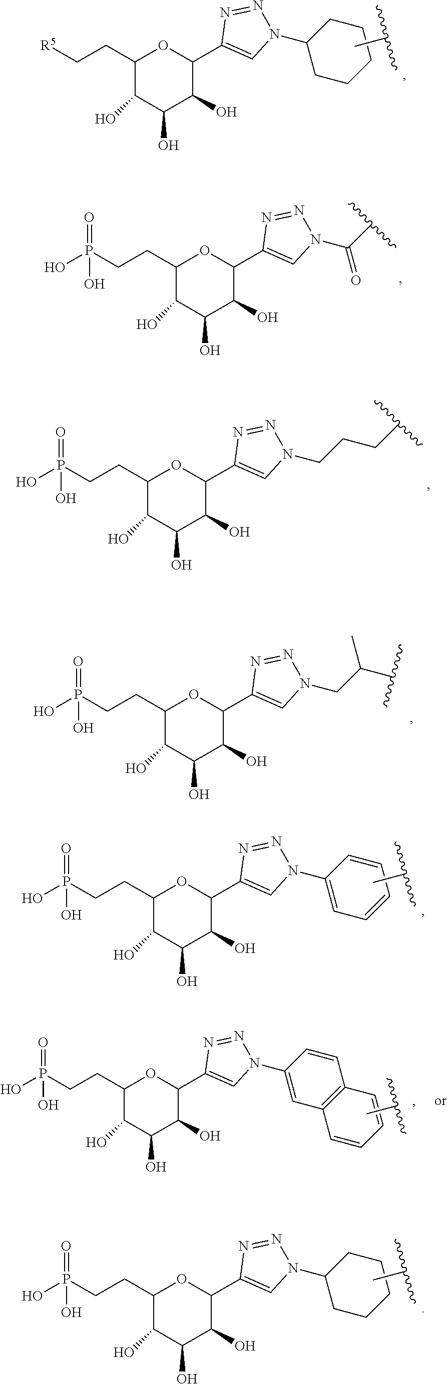

In certain embodiments the Mannose 6-Phosphate Ligand binds the cation independent mannose 6-phosphate receptor (which is also known as CI-MPR, IGF2 receptor, or CD222). In other embodiments the Mannose 6-Phosphate Ligand binds the cation dependent mannose 6-phosphate receptor (which is also known as CD-MPR and the 46 kDa mannose 6-phosphate receptor). In certain embodiments the Mannose 6-Phosphate Ligand binds to both the cation independent mannose 6-phosphate receptor and the cation dependent mannose 6-phosphate receptor.

In an embodiment of the invention, the Extracellular Protein Targeting Ligand is a small organic molecule (i.e., a non-biologic) that adequately binds to the protein in such a manner that it is able to transport it to the mannose 6-phosphate receptor. In other embodiments the Extracellular Protein Targeting Ligand is a residue of a pharmaceutically active compound that binds to the target extracellular protein (for example but not limited to a compound of the sort that would be reviewed as a drug by CDER of the FDA, or an approved or clinical stage drug), a peptide, a protein, a biologic, or a binding fragment thereof that adequately binds to the protein in such a manner that it is able to transport it to the mannose 6-phosphate receptor. A plethora of illustrative nonlimiting examples of Extracellular Protein Targeting Ligands is provided in FIG. 1.

In certain embodiments the extracellular protein is a membrane bound protein.

The Extracellular Protein Degraders of the present invention can be administered in any manner that allows the degrader to bind to the Extracellular Protein, typically in the blood stream, and carry it to a mannose 6-phosphate receptor bearing cell for endocytosis and degradation. As such, examples of methods to deliver the degraders of the present invention include, but are not limited to, oral, intravenous, buccal, sublingual, subcutaneous and transnasal.

In other aspects of the present invention new mannose 6-phosphate receptor ligands are provided of Formula IV:

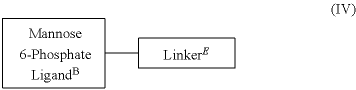

wherein:
Mannose 6-Phosphate Ligand^{B} is selected from:

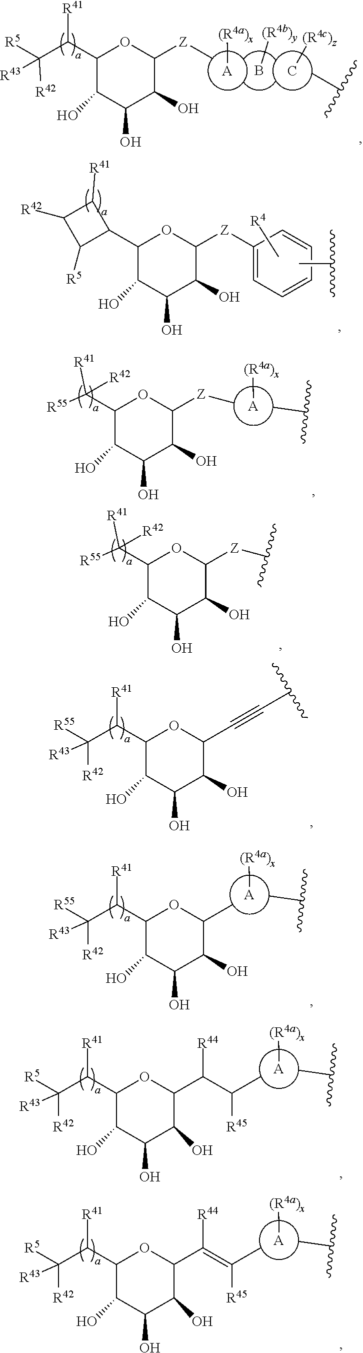

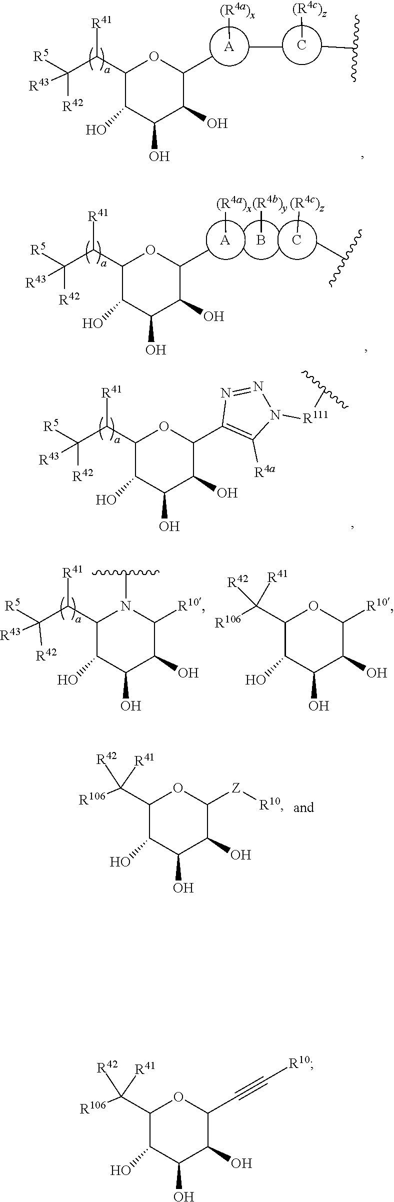

Linker^{E} is selected from

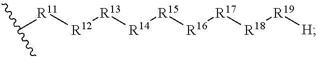

R¹¹, R¹², R¹³, R¹⁴, R¹⁵, R¹⁶, R¹⁷, R¹⁸, and R¹⁹ are independently at each occurrence selected from the group consisting of a bond, alkyl, —C(O)—, —C(O)O—, —OC(O)—, —SO₂—, —S(O)—, —C(S)—, —C(O)NR⁶—, —NR⁶C(O)—, —C(O)N(OH)—, —N(OH)C(O)—, —O—, —S—, —NR⁶—, —C(R²¹R²¹)—, —S(O)(═N—R⁴)—, —S(O)(═NR), —P(O)(R³)O—, —P(O)(R³)—, a divalent residue of a natural or unnatural amino acid, alkenyl, alkynyl, haloalkyl, alkoxy, aryl, heterocycle, heteroaryl, —CH₂CH₂—[O—(CH₂)₂]ₙ—O—, —CH₂CH₂—[O—(CH₂)₂]ₙ—NR⁶—, —CH₂CH₂—[O—(CH₂)₂]ₙ—, -[—(CH₂)₂—O-]ₙ-, [O—(CH₂)₂]ₙ—, —[O—CH(CH₃)C(O)]ₙ—, —[C(O)—CH(CH₃)—O]ₙ—, —[O—CH₂C(O)]ₙ—, —[C(O)—CH₂—O]ₙ—, a divalent residue of a fatty acid, a divalent residue of an unsaturated or saturated mono- or dicarboxylic acid; each of which is optionally substituted with 1, 2, 3, or 4 substituents independently selected from R²¹;
n is independently selected at each instance from 0, 1, 2, 3, 4, 5, 6, 7, 8, 9, or 10;
R²¹ is independently at each occurrence selected from the group consisting of hydrogen, alkyl, alkenyl, alkynyl, F, Cl, Br, I, hydroxyl, alkoxy, azide, amino, cyano, —NR⁶R⁷, —NR⁸SO₂R³, —NR⁸S(O)R³, haloalkyl, aryl, heteroaryl, and heterocycle;
R¹¹¹ is aryl, heteroaryl, heterocycle, C₃-C₁₀alkyl, alkenyl, or alkynyl each of which is optionally independently substituted by 1, 2; or 3 R⁴⁶ substituents;
R¹⁰⁶ is selected from

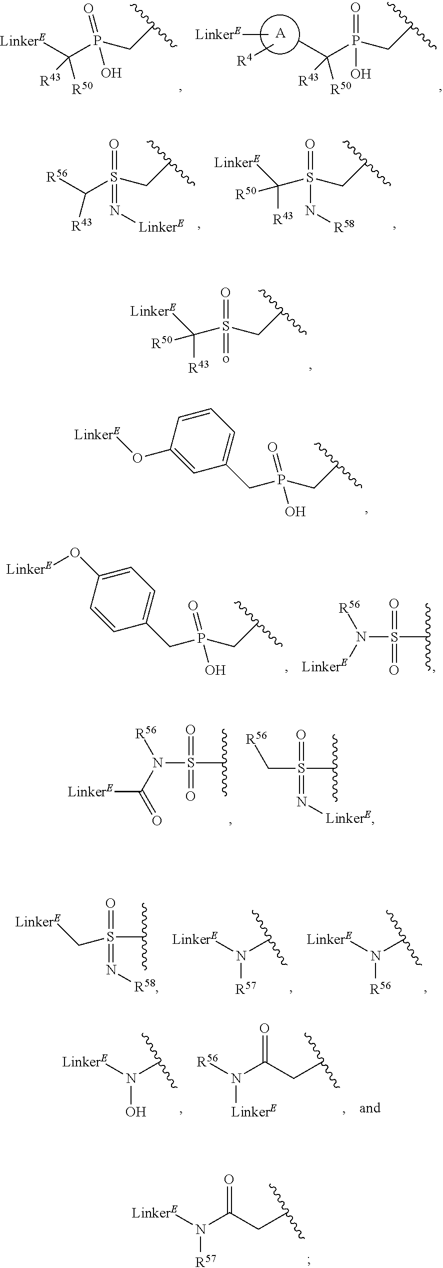

all other variables are as defined herein and wherein the optional substituents are selected such that a stable compound results for example a compound of Formula IV with a shelf stability of at least 1, 2, 3, 4, 5, 6 or more months.

The compounds of Formula IV can be used as intermediates in the manufacture of compounds of the present invention, probe molecules for assessing the effect of binding the cation independent or cation dependent mannose 6-phosphate receptor, therapeutics for the treatment of diseases mediated by mannose 6-phosphate, or for other uses.

In certain aspects one or more of the mannose 6-phosphate receptor ligands is an oligomer. For example, in certain embodiments the compound of the present invention is selected from:

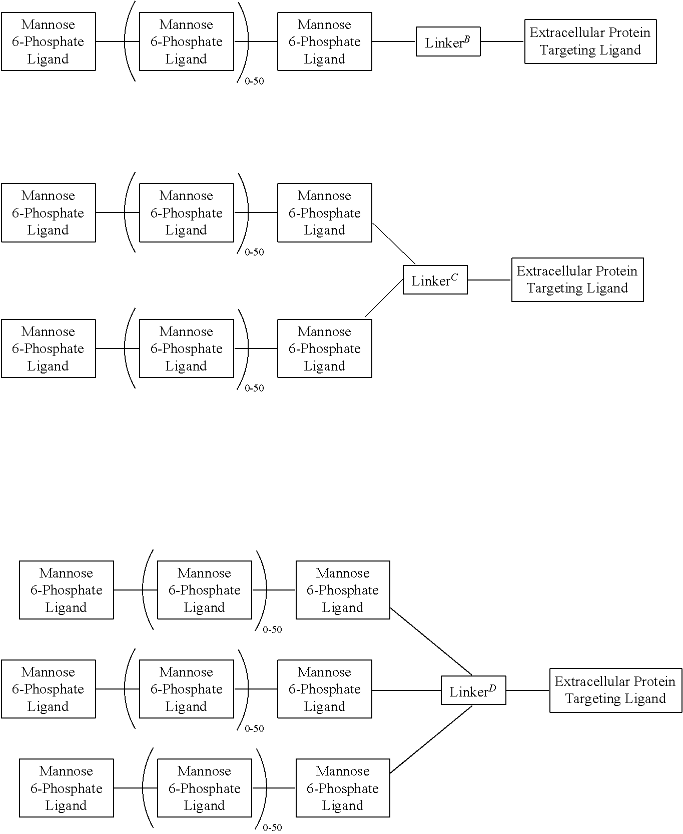

When presented as an oligomer the mannose 6-phosphate receptor ligand may only have the phosphate group or analog thereof on the terminal position or may have the phosphate group or analog thereof in between each sugar moiety. For example, oligomers of Formula I include both:

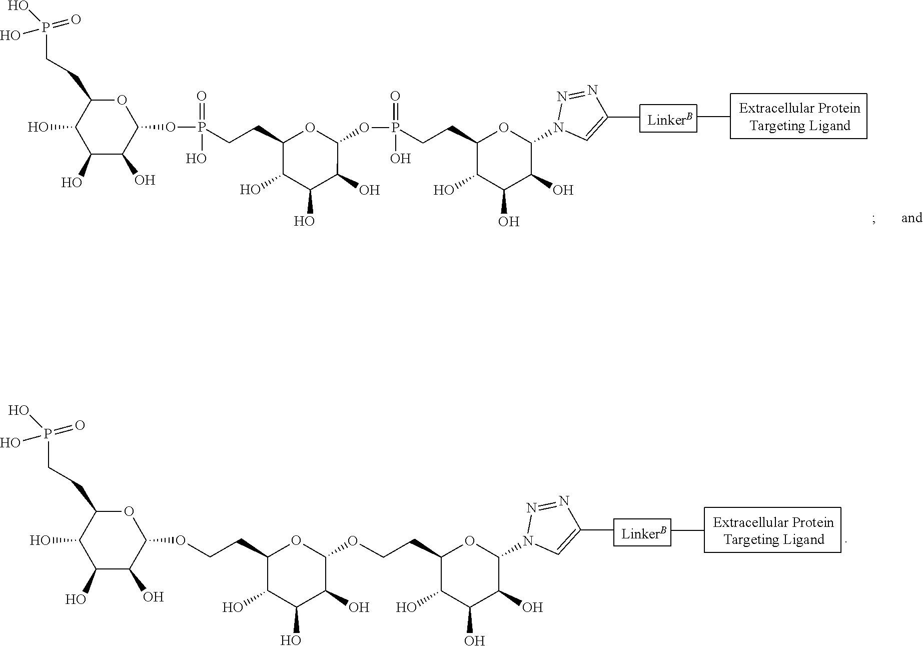

### BRIEF DESCRIPTION OF FIGURES

FIG. 1A provides a non-limiting list of Extracellular Protein Targeting Ligands that target Immunoglobulin A (IgA).

FIG. 1B provides a non-limiting list of Extracellular Protein Targeting Ligands that target Immunoglobulin G (IgG).

FIGS. 1C-1G provides a non-limiting list of Extracellular Protein Targeting Ligands that target Immunoglobulin E (IgE).

FIGS. 1H-1M provides a non-limiting list of Extracellular Protein Targeting Ligands that target Tumor Necrosis Factor alpha (TNF-α).

FIG. 1N provides a non-limiting list of Extracellular Protein Targeting Ligands that target Interleukin-1 (IL-1).

FIGS. 10-1S provides a non-limiting list of Extracellular Protein Targeting Ligands that target Interleukin-2 (IL-2).

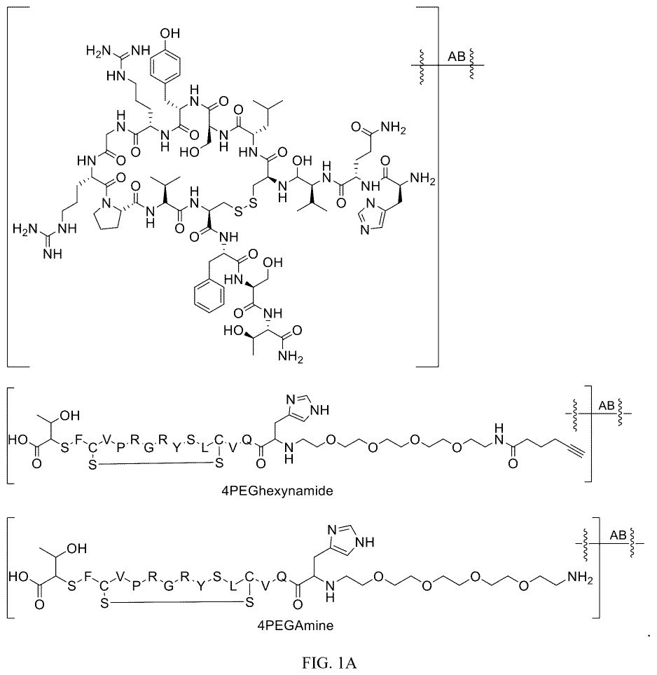

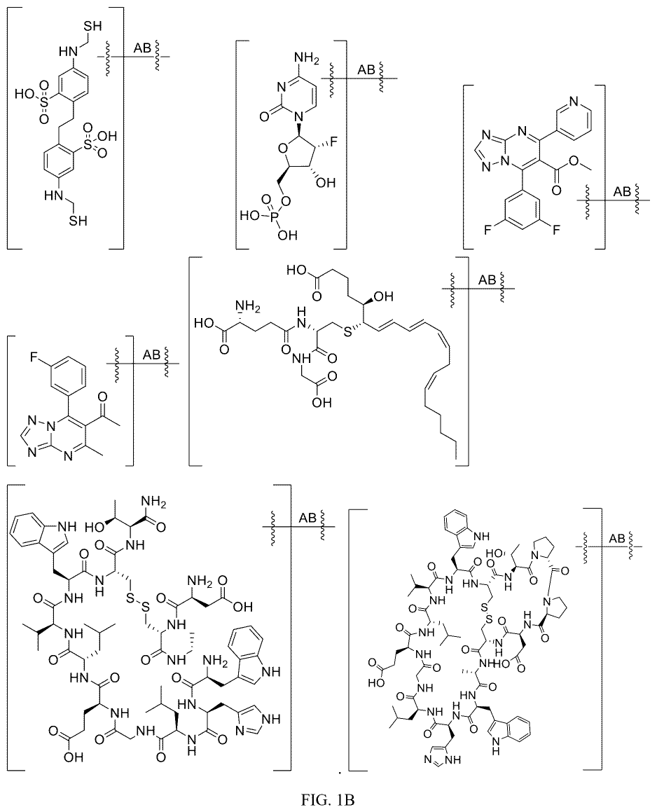

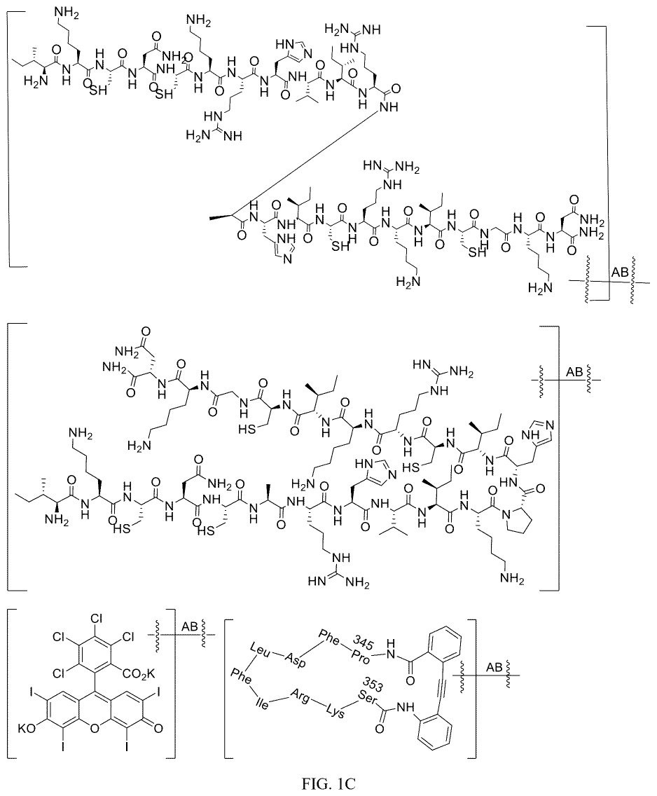

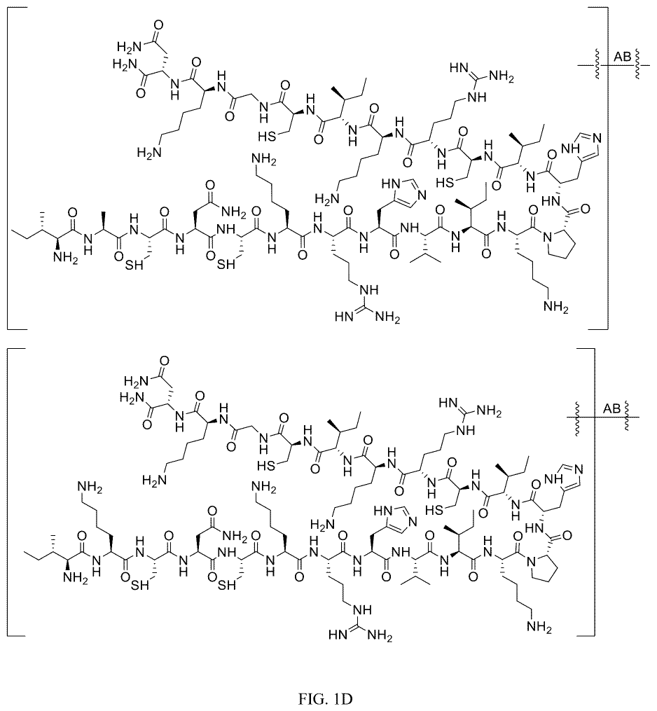

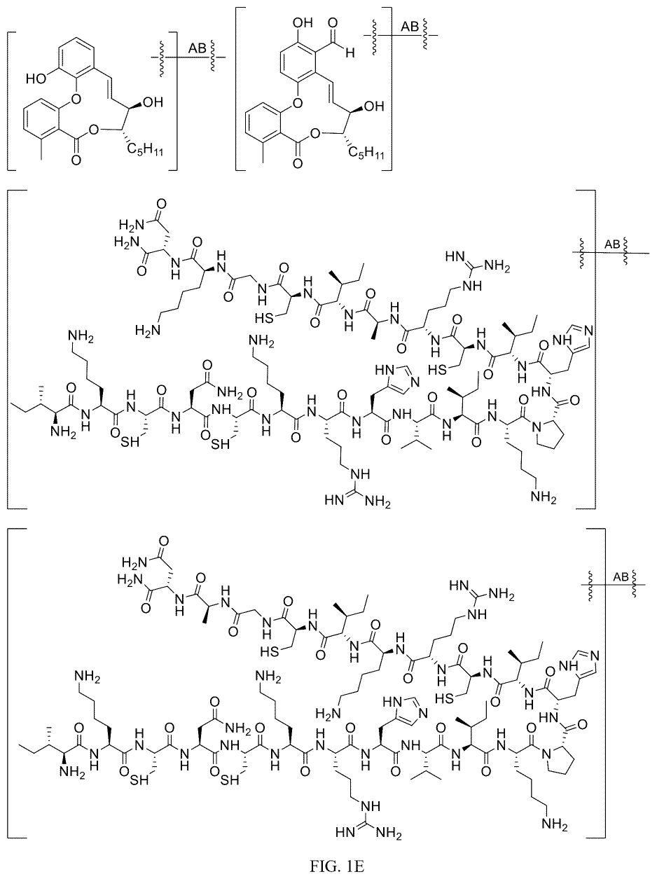

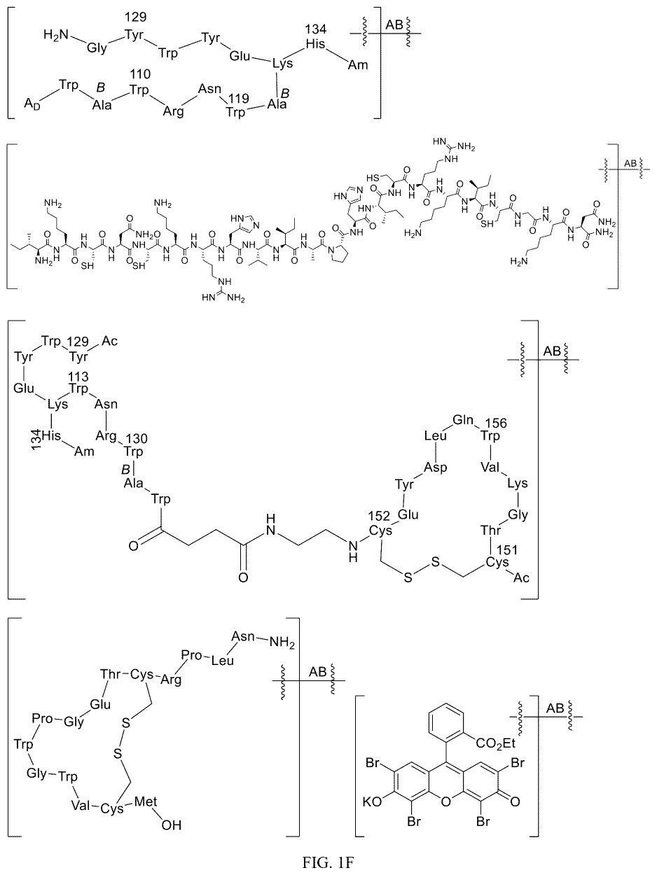

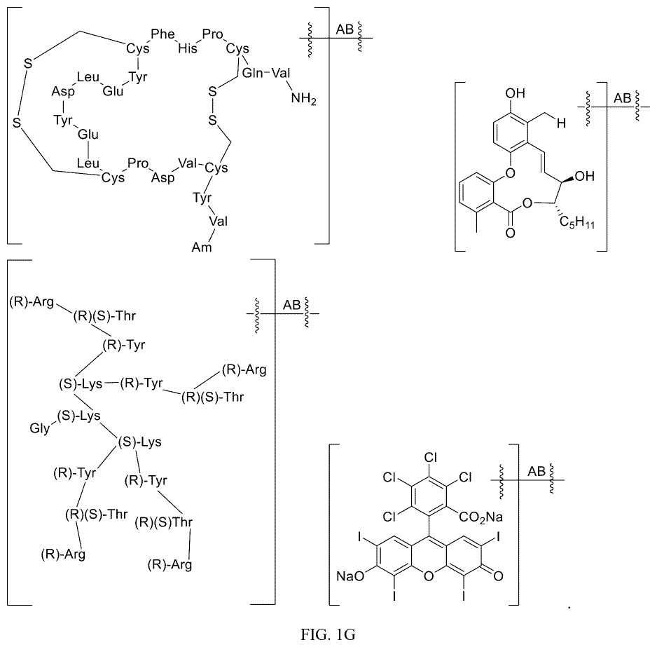

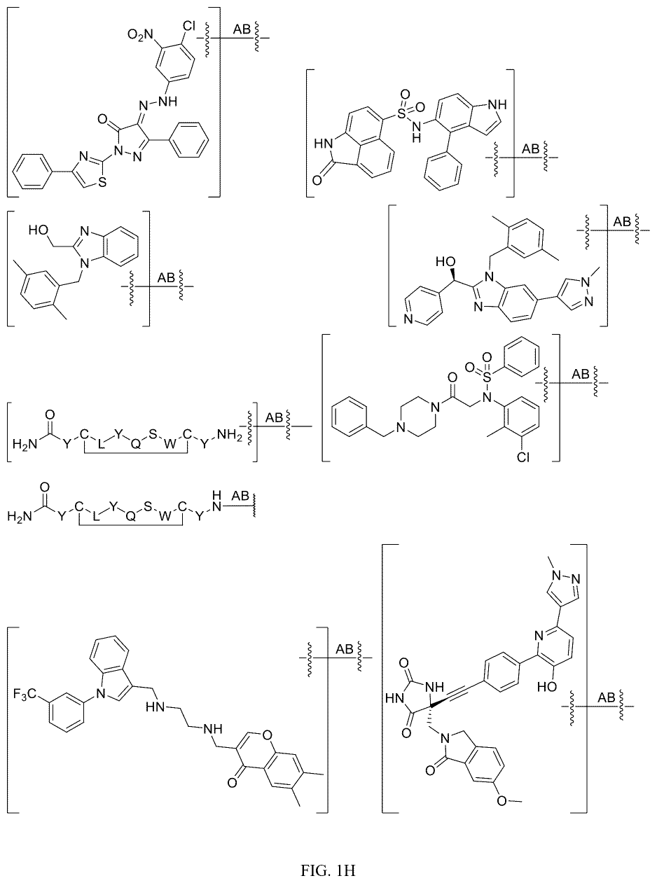

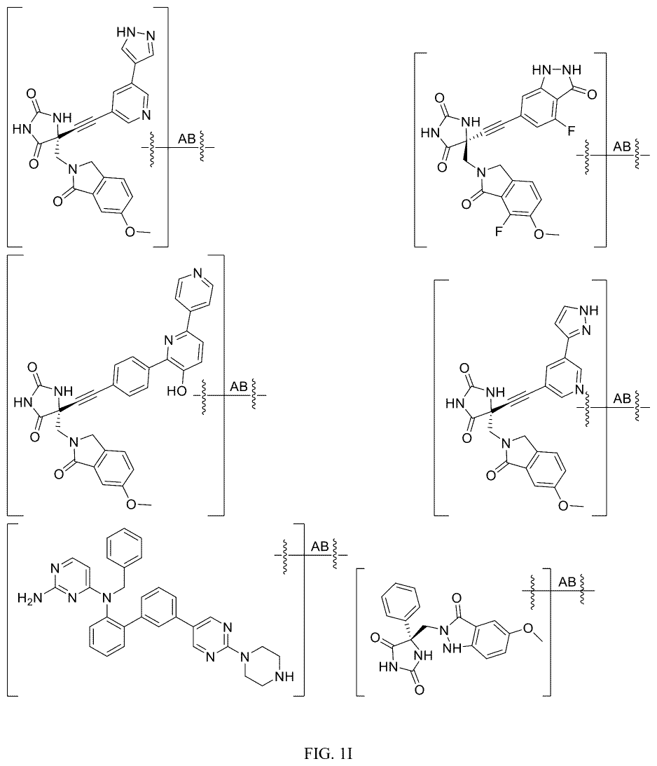

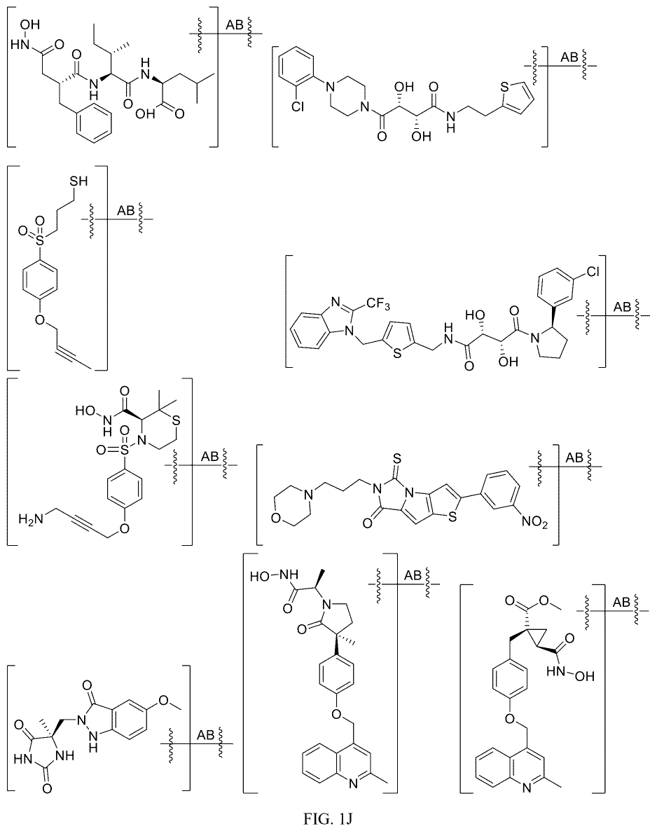

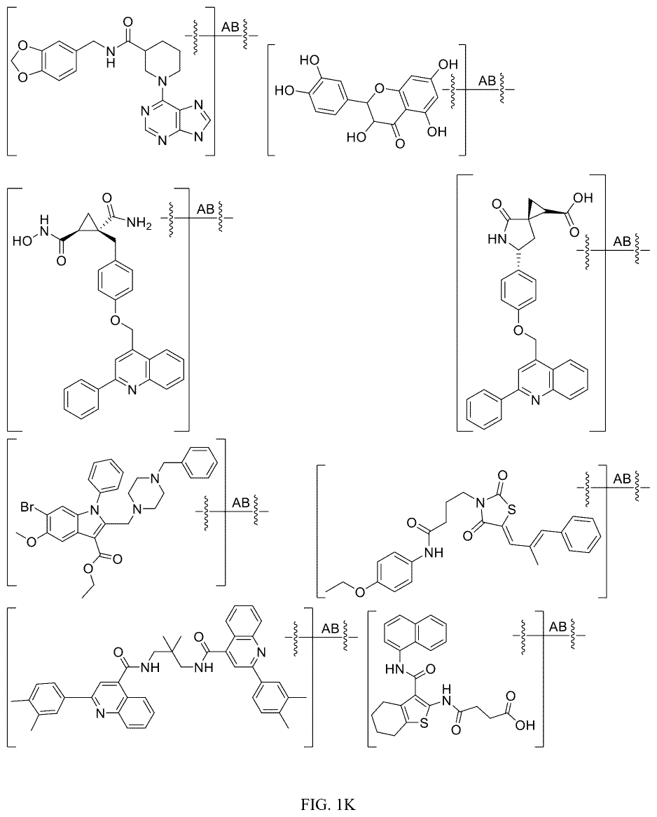

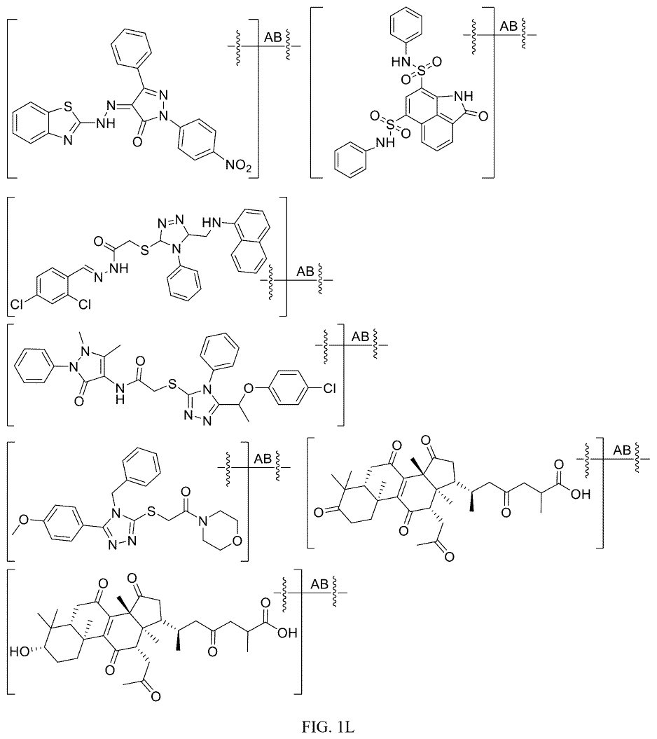

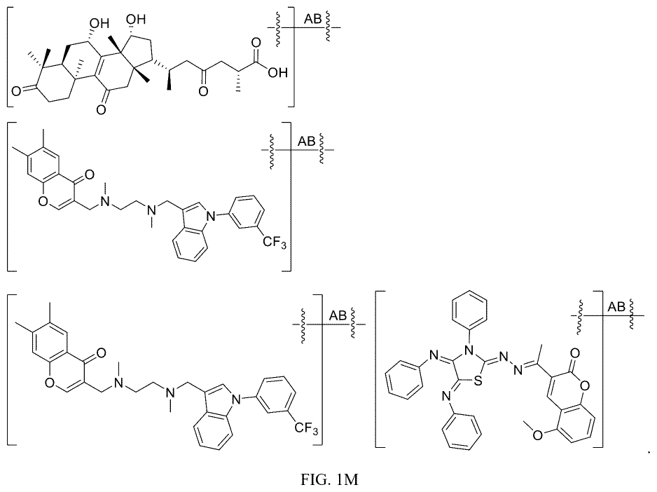

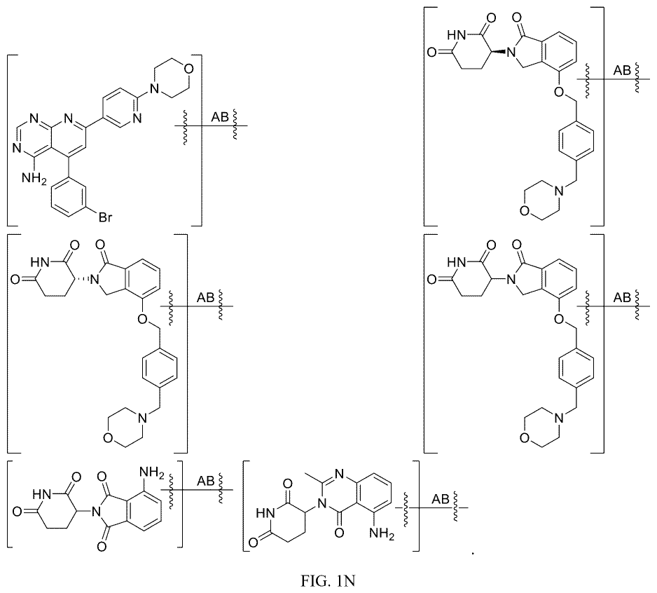

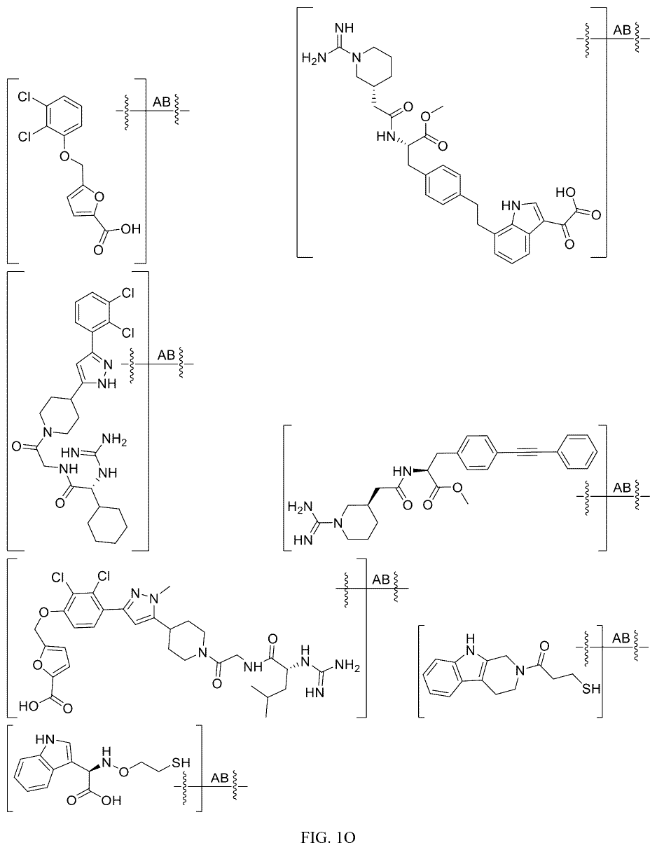

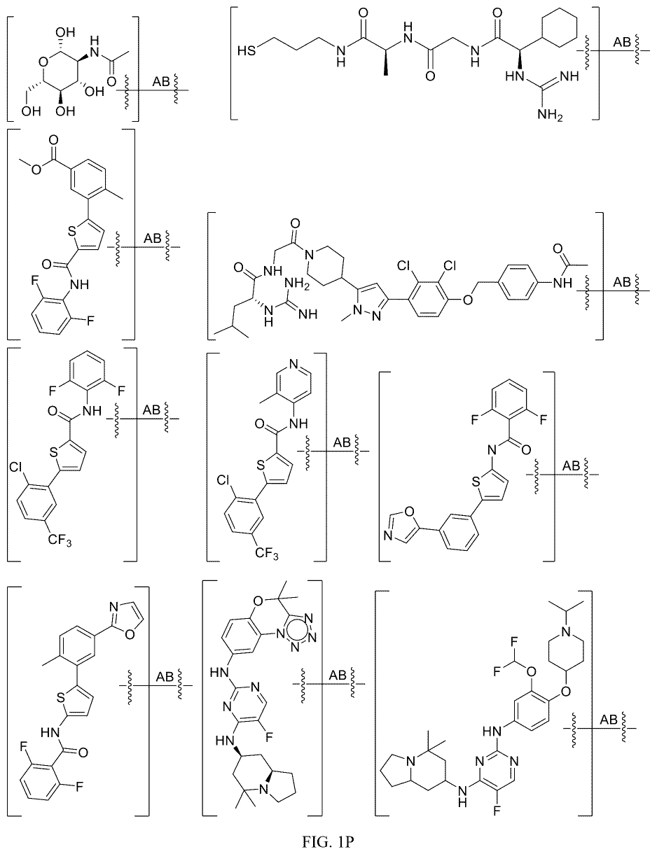

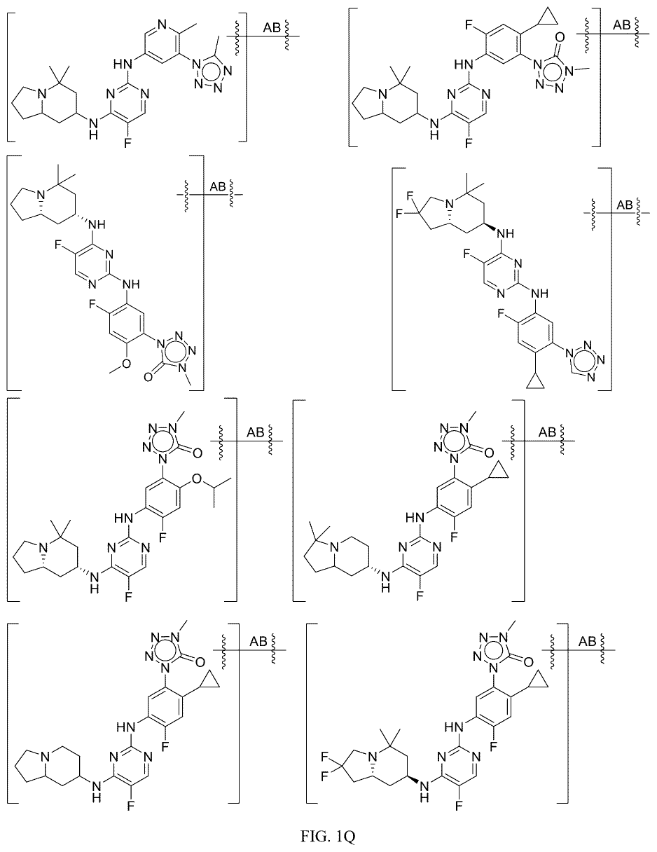

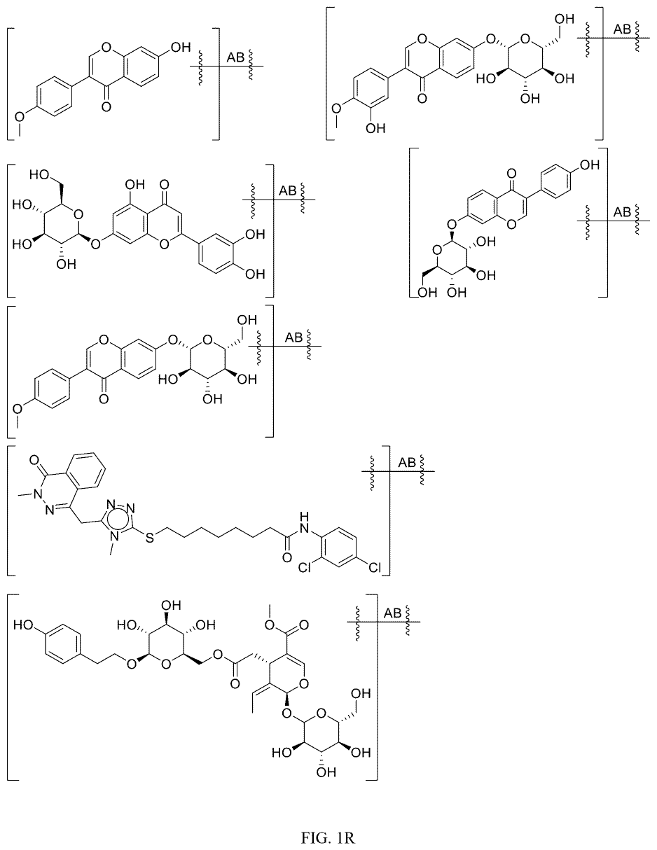

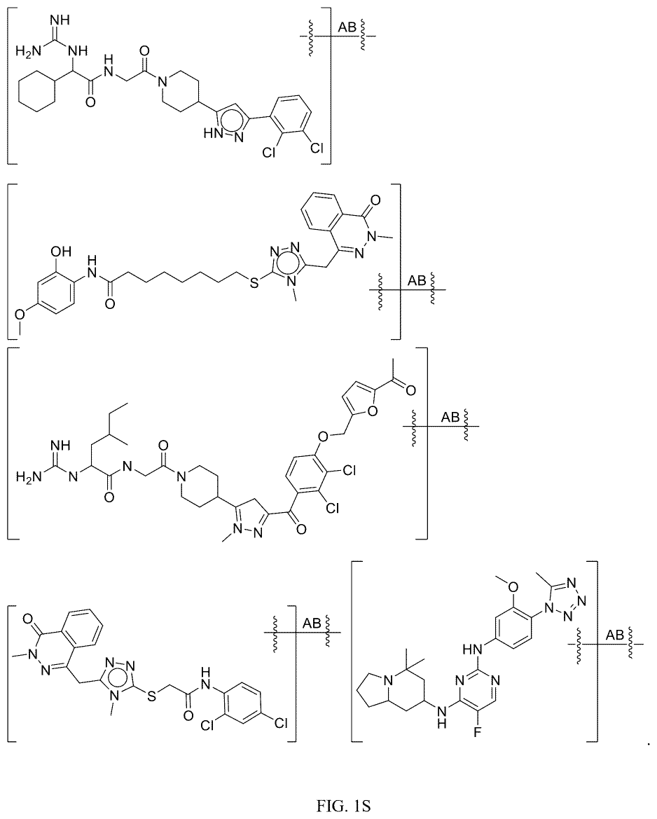

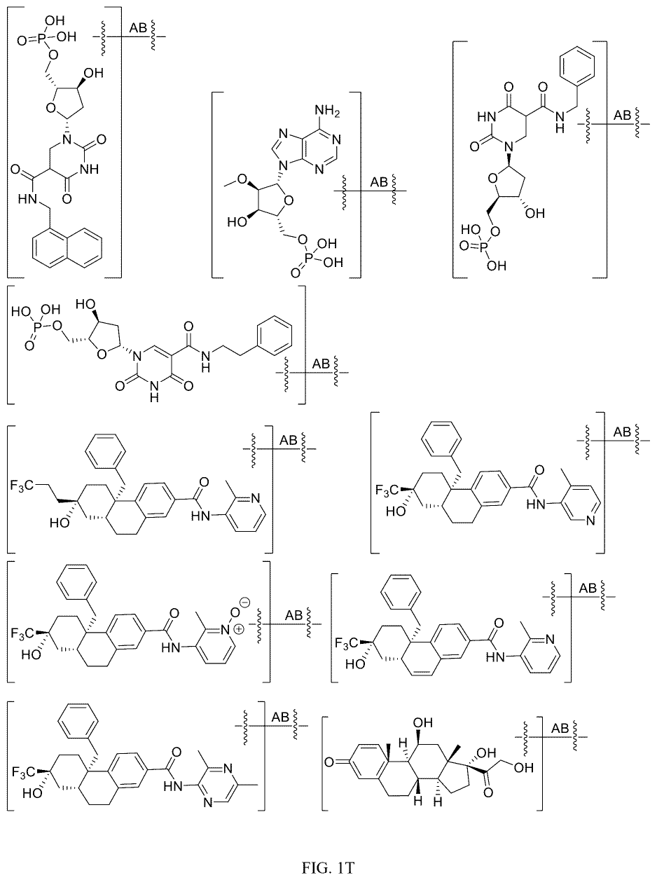

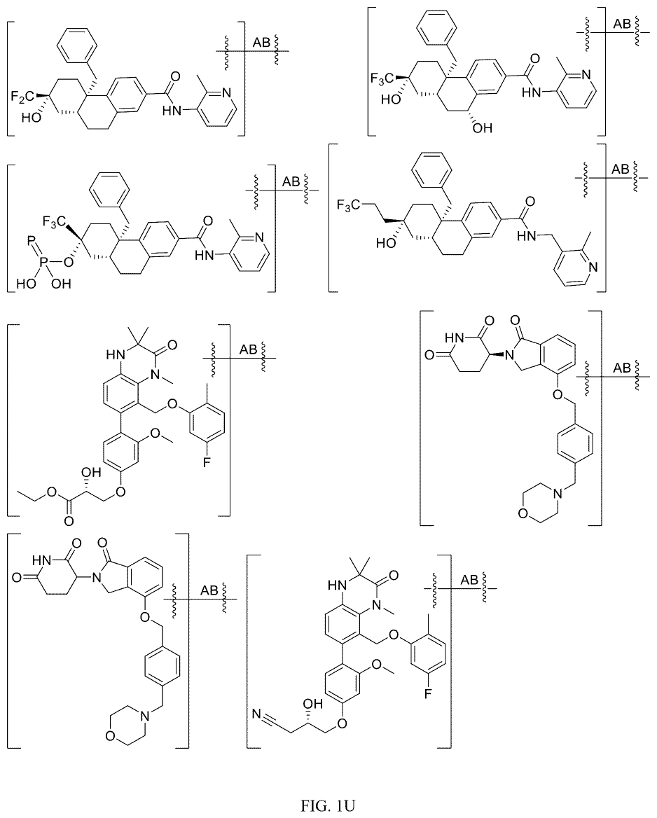

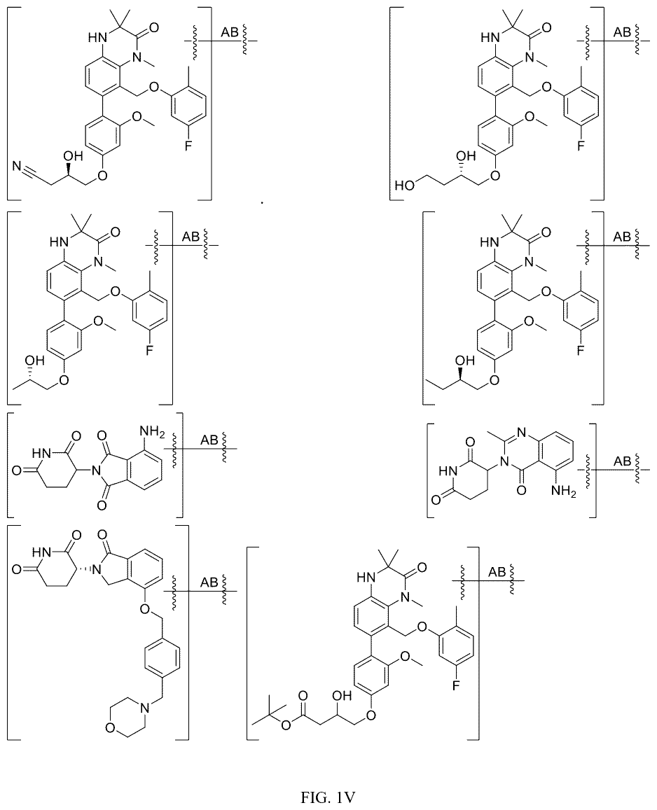

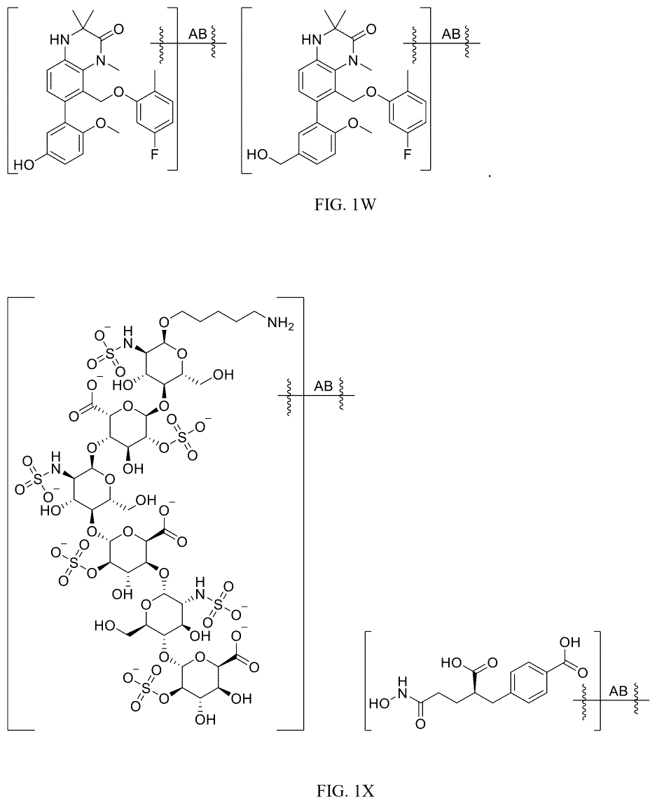

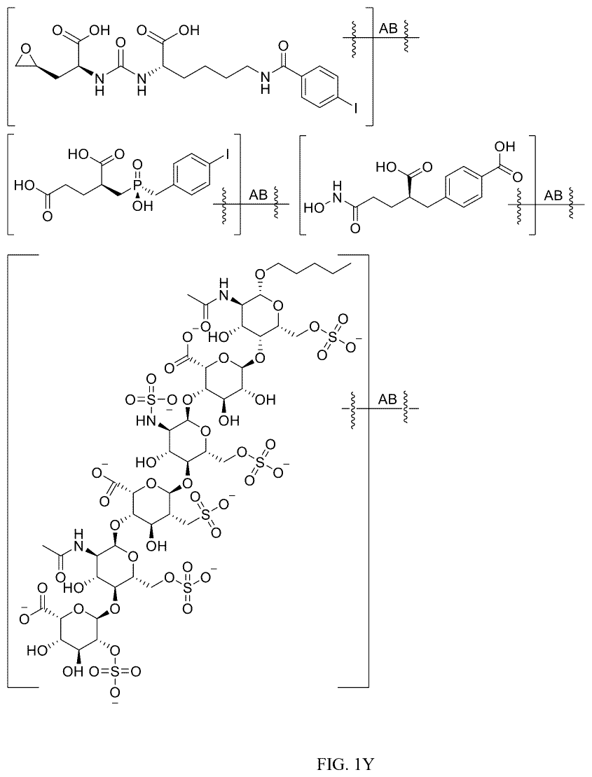

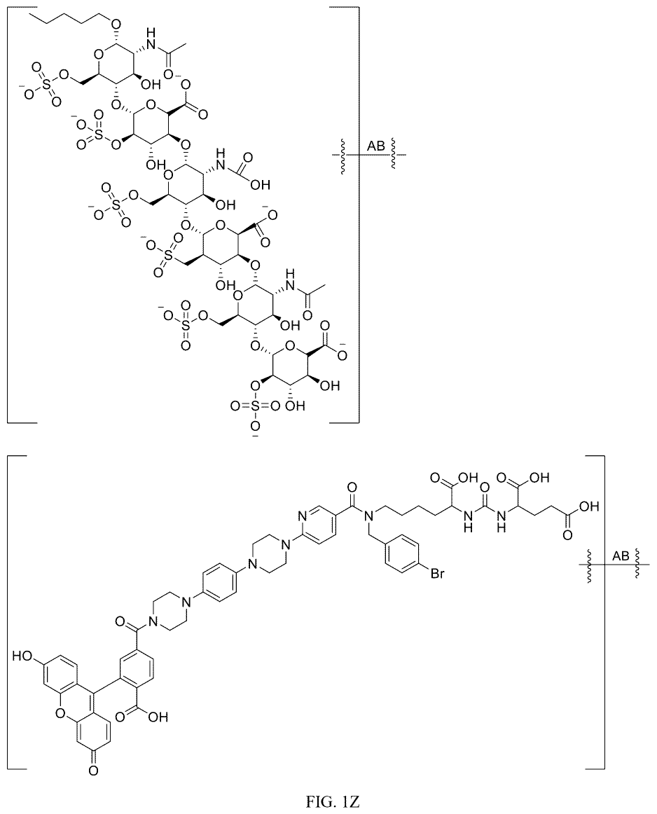

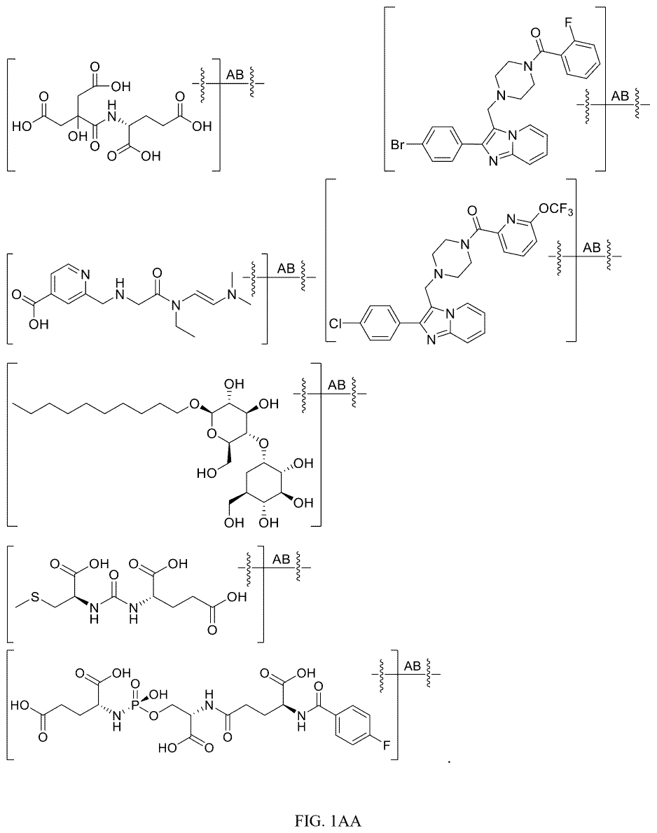

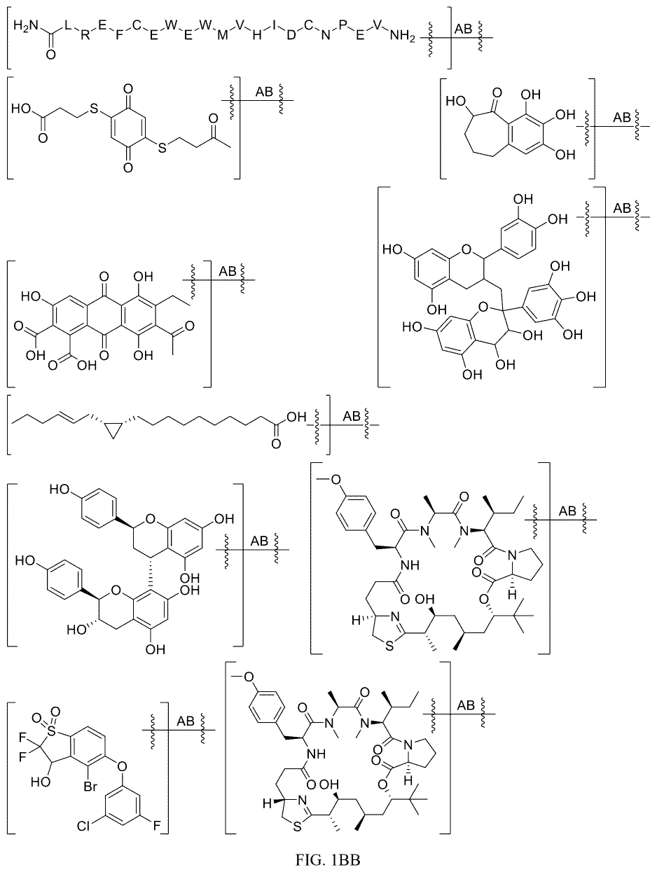

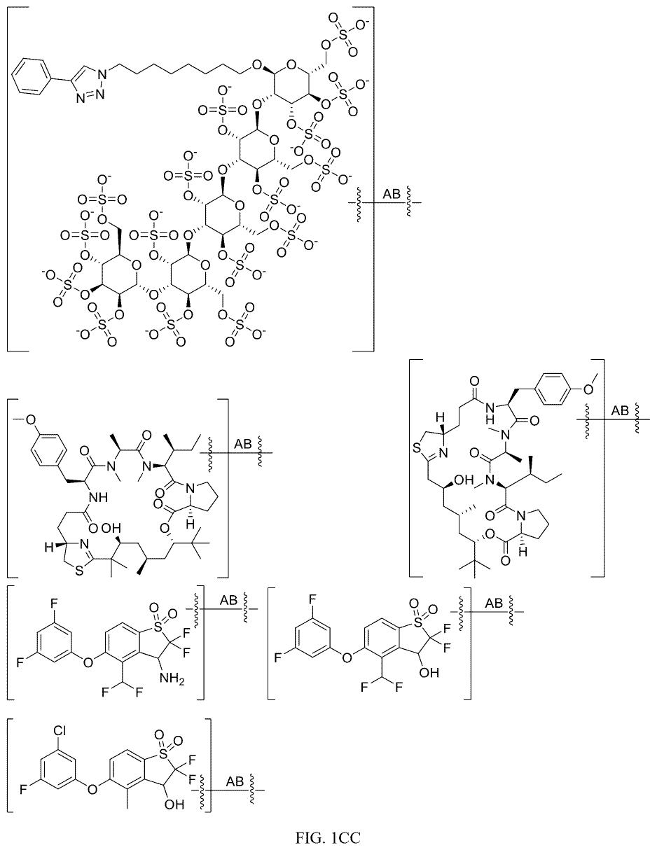

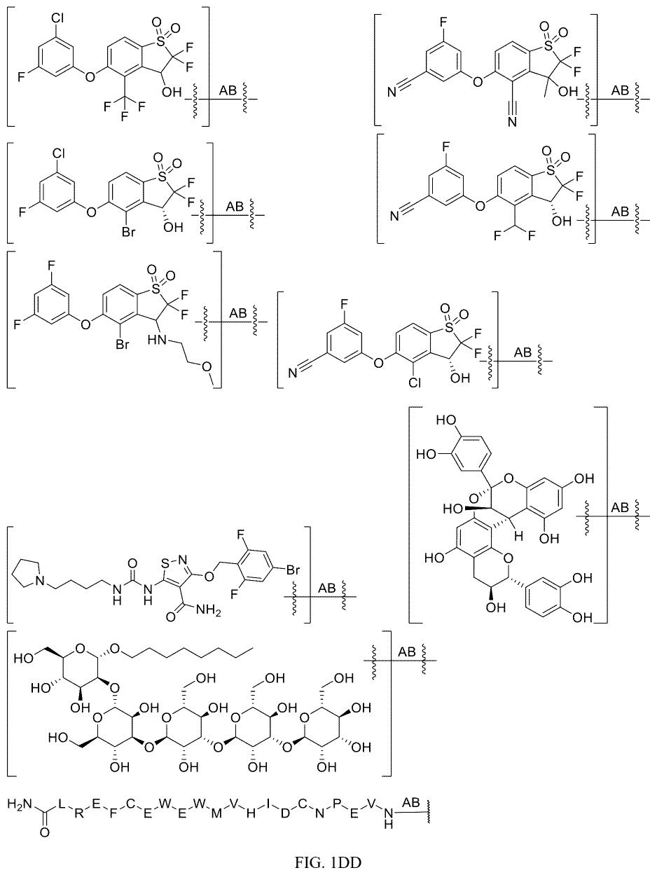

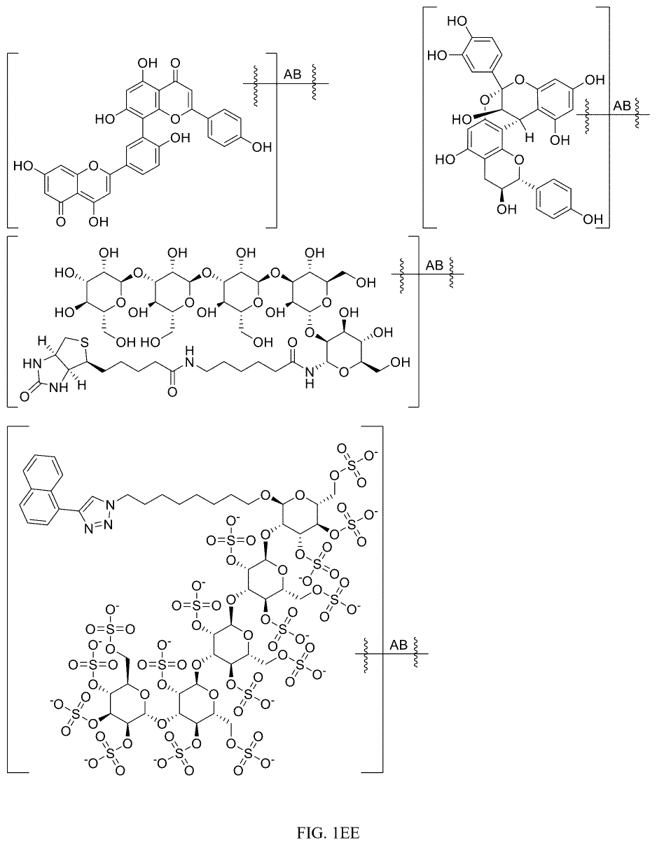

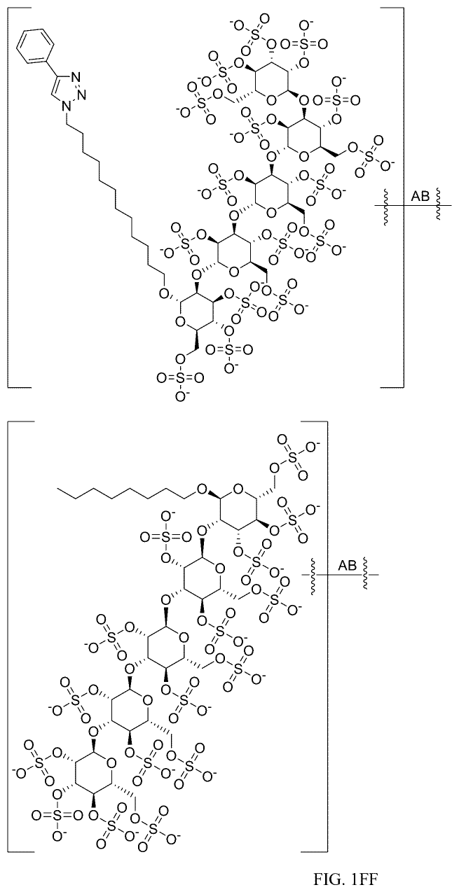

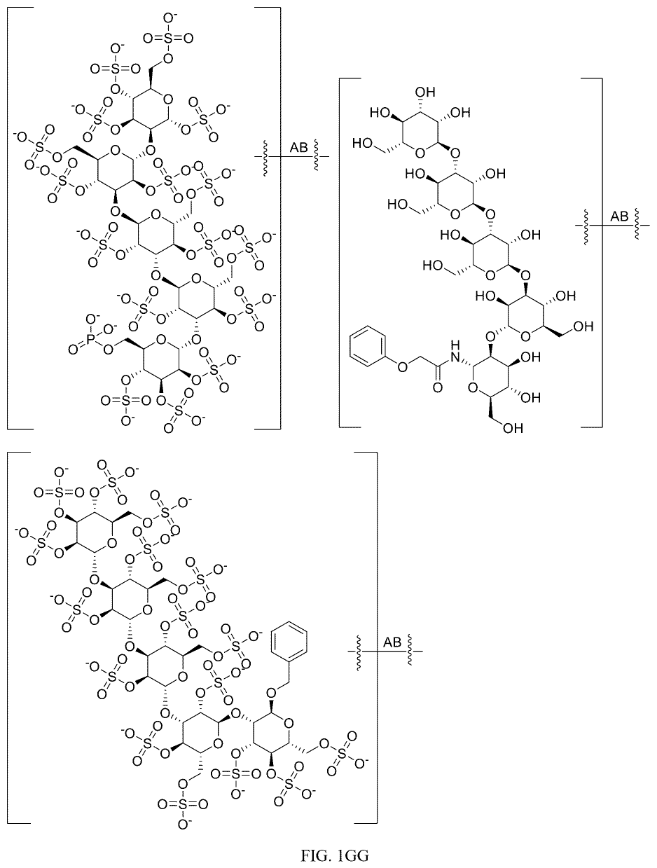

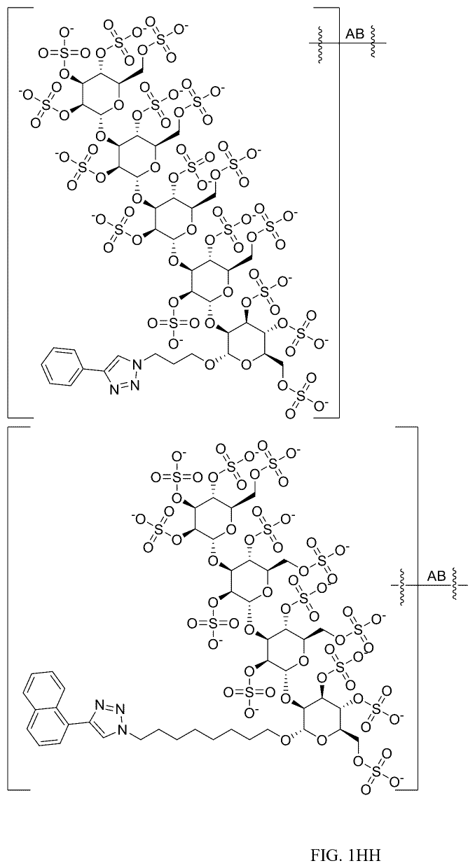

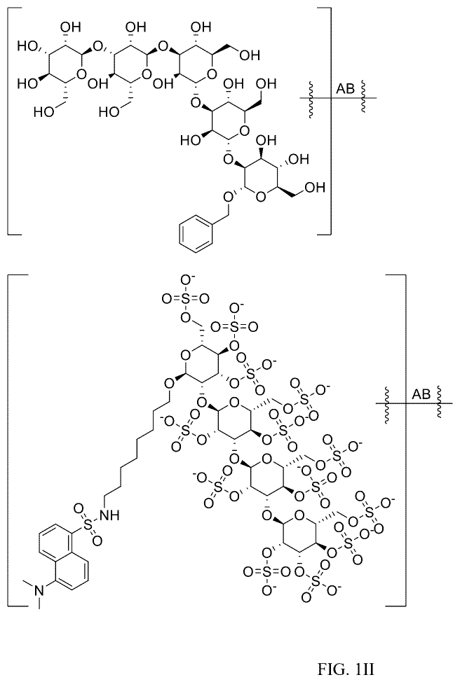

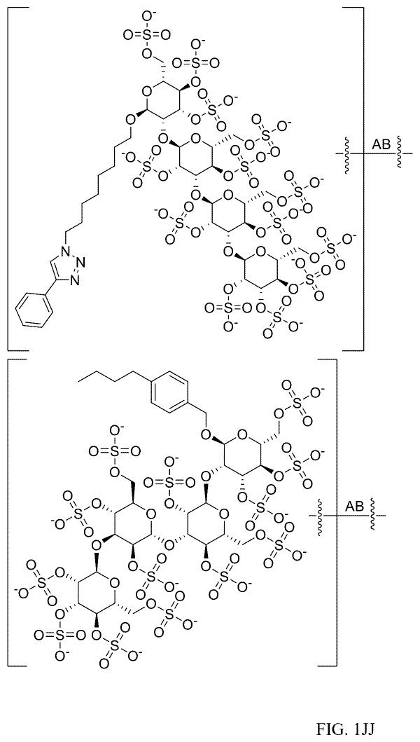

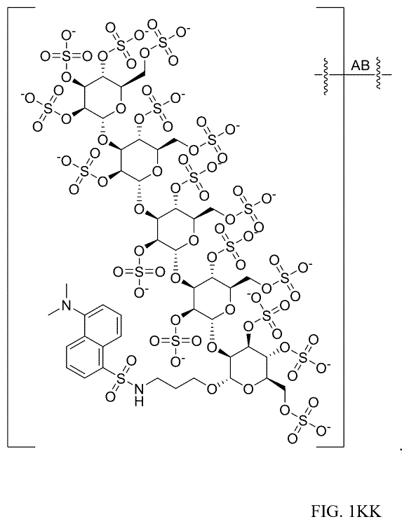

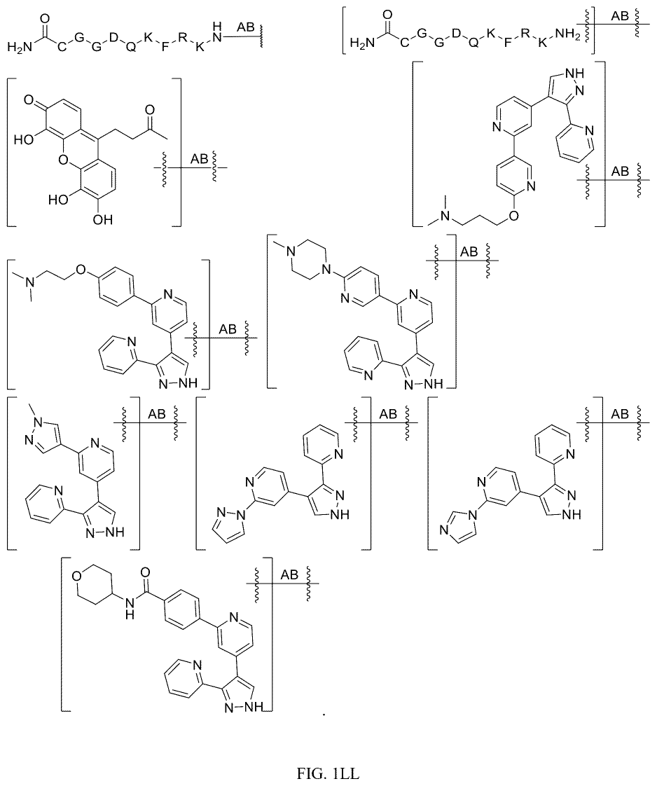

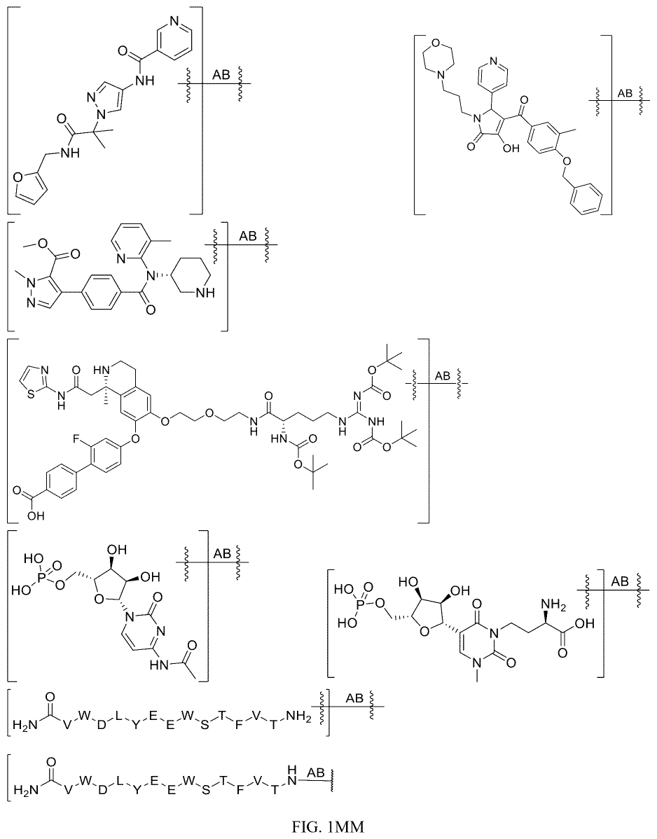

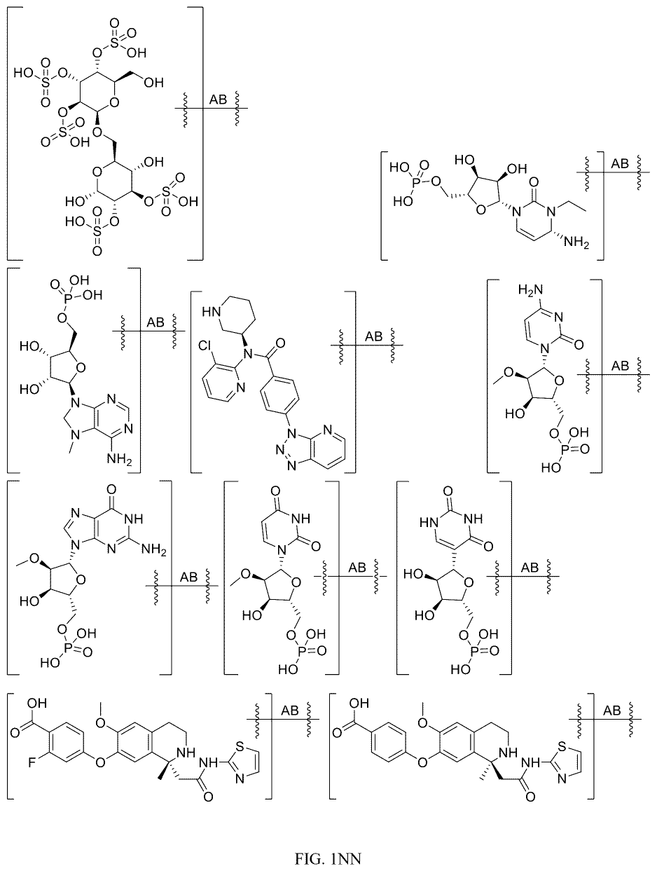

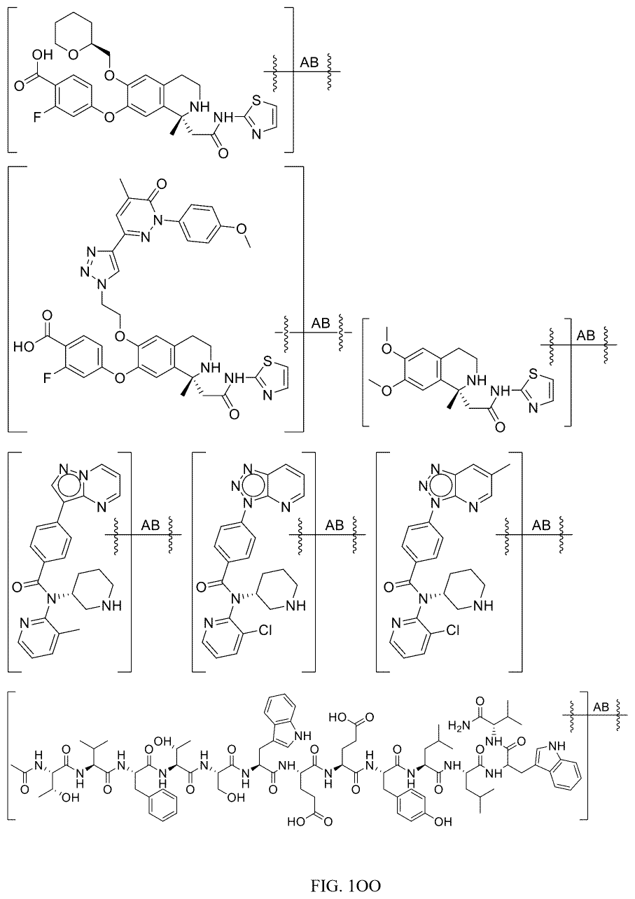

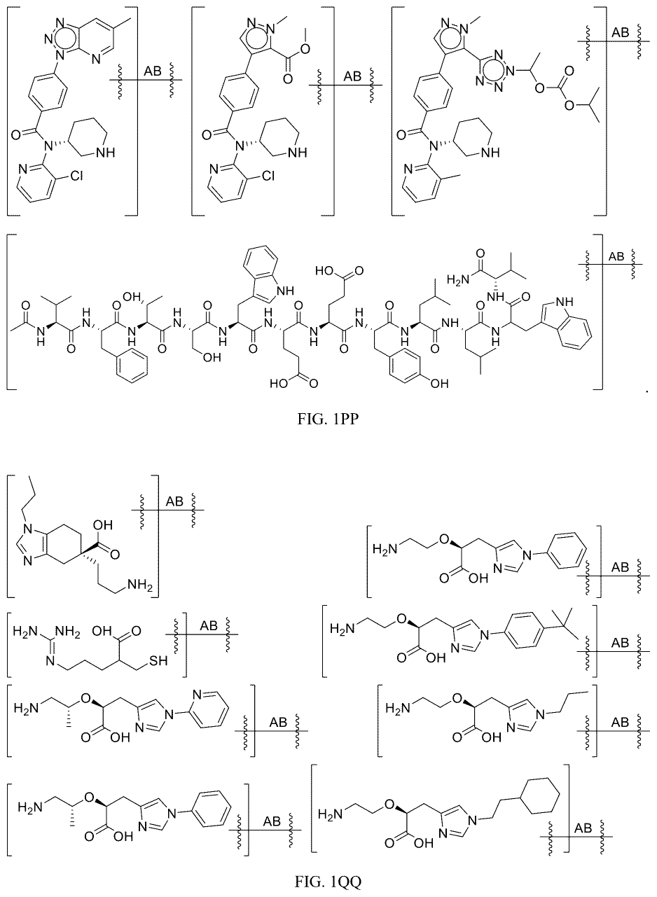

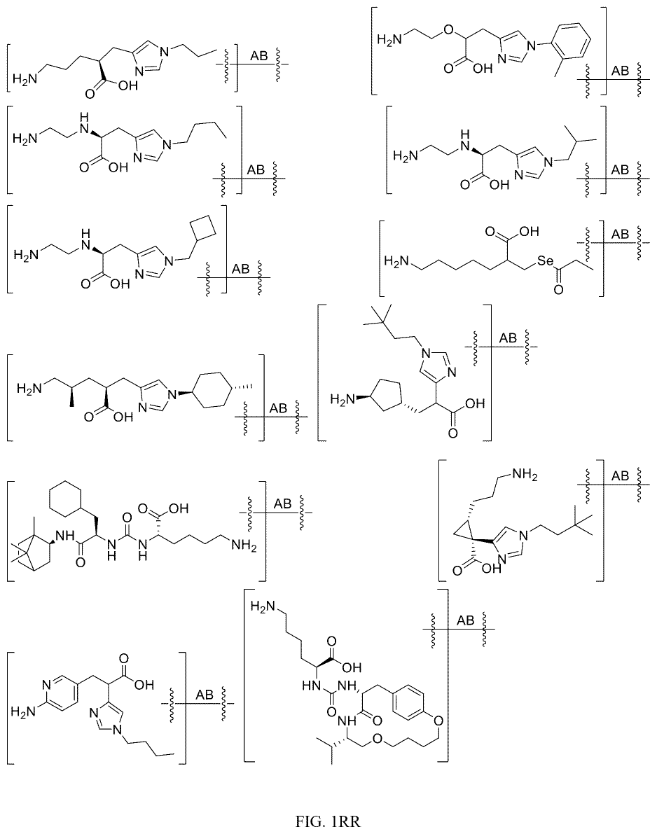

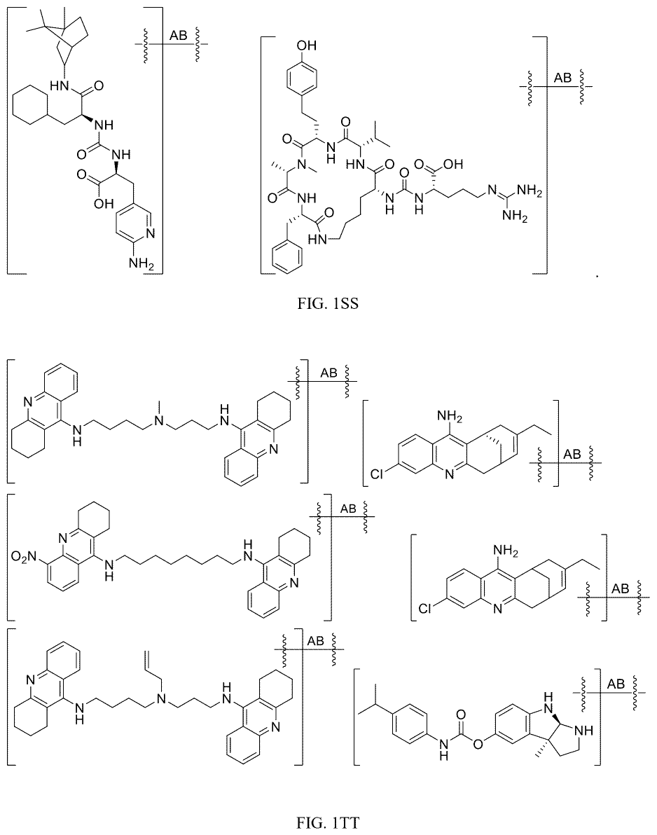

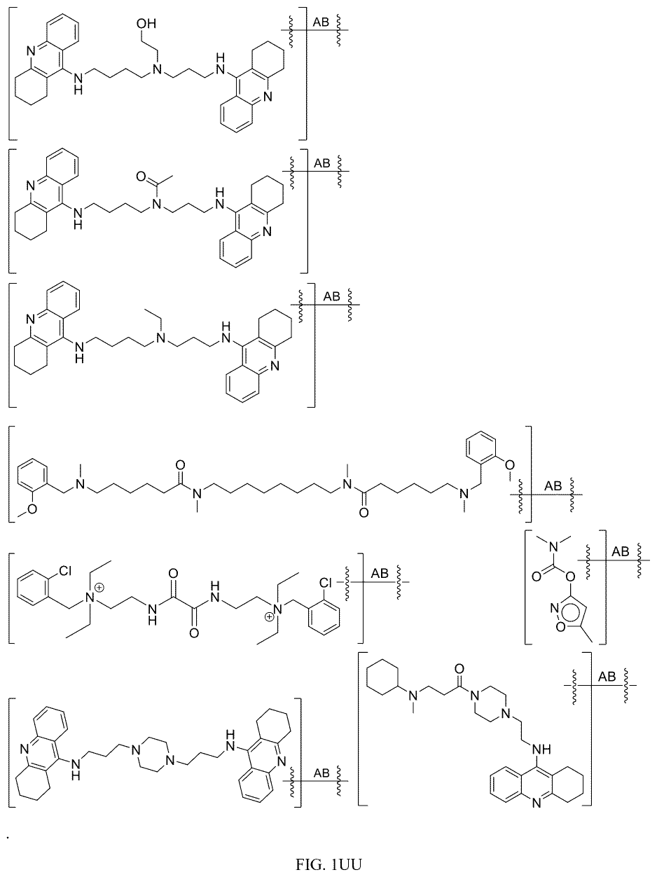

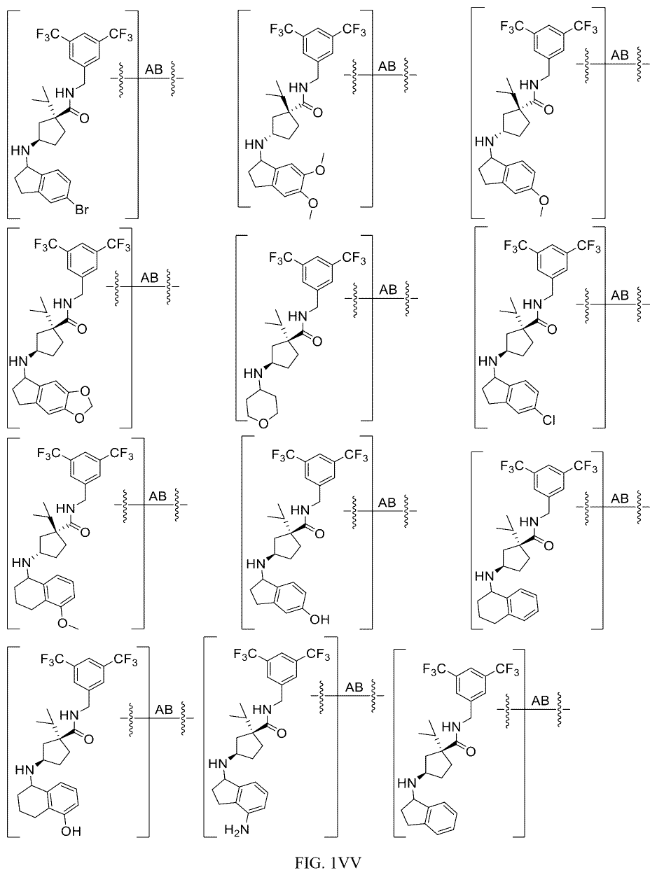

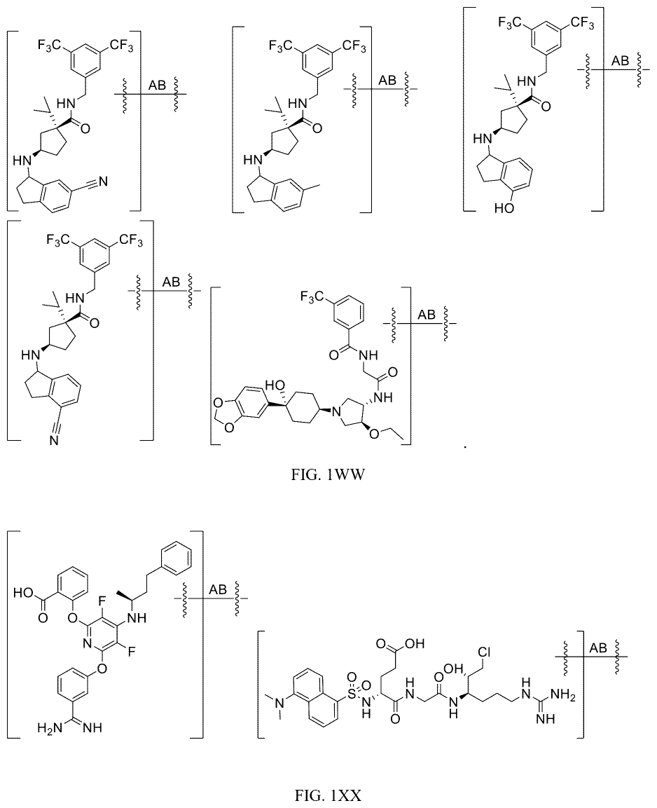

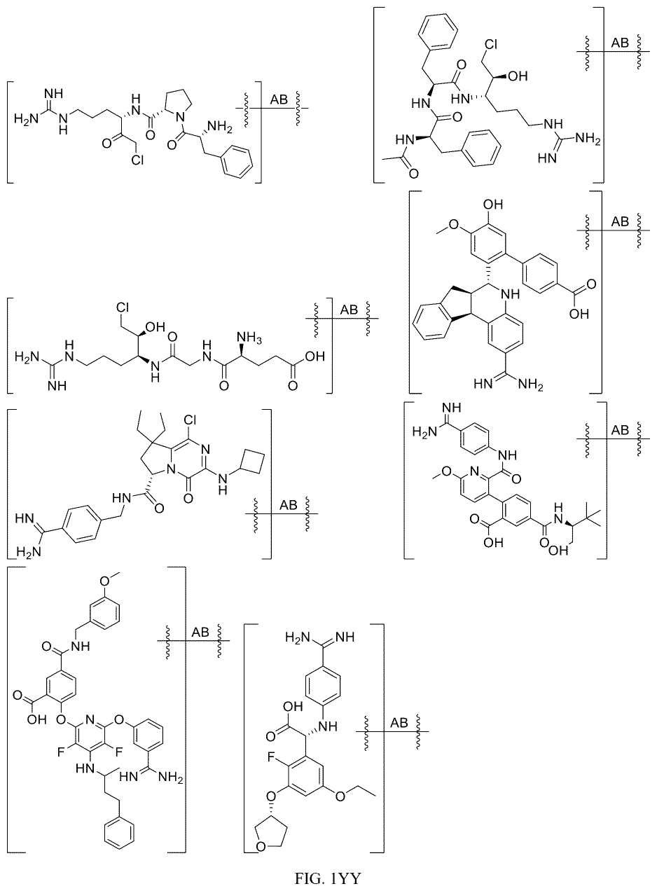

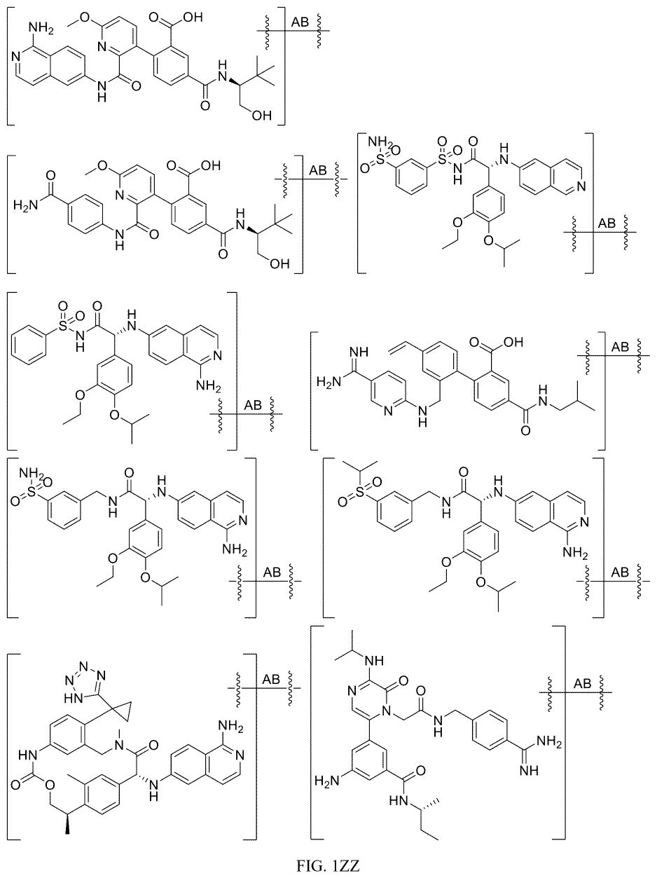

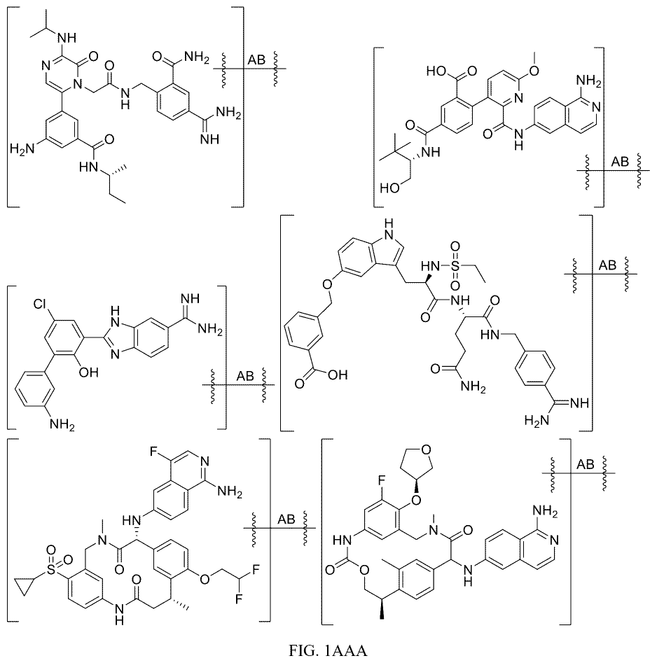

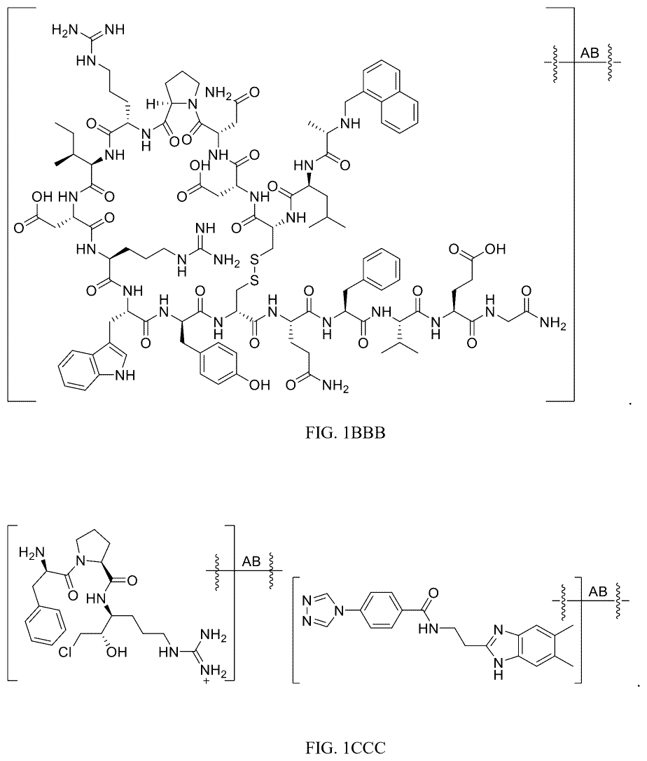

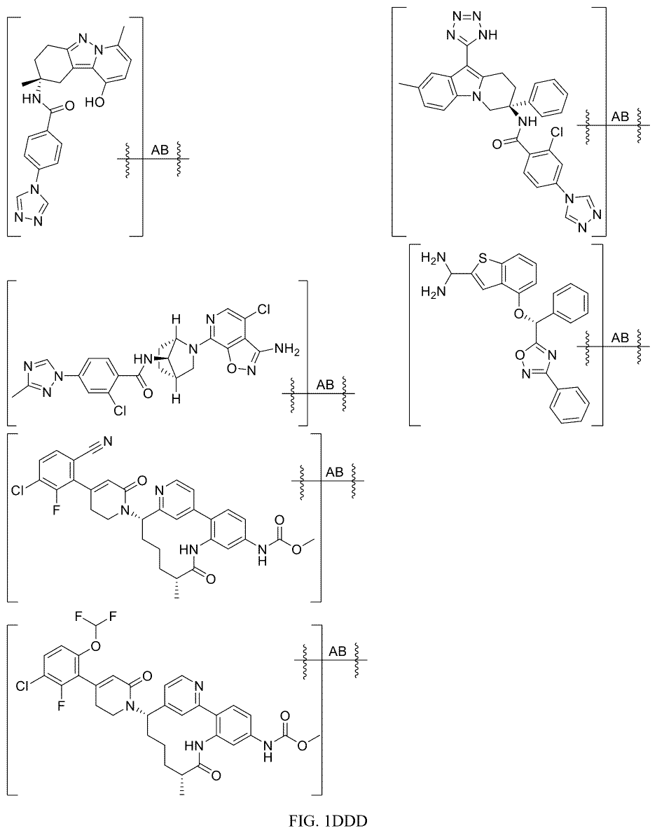

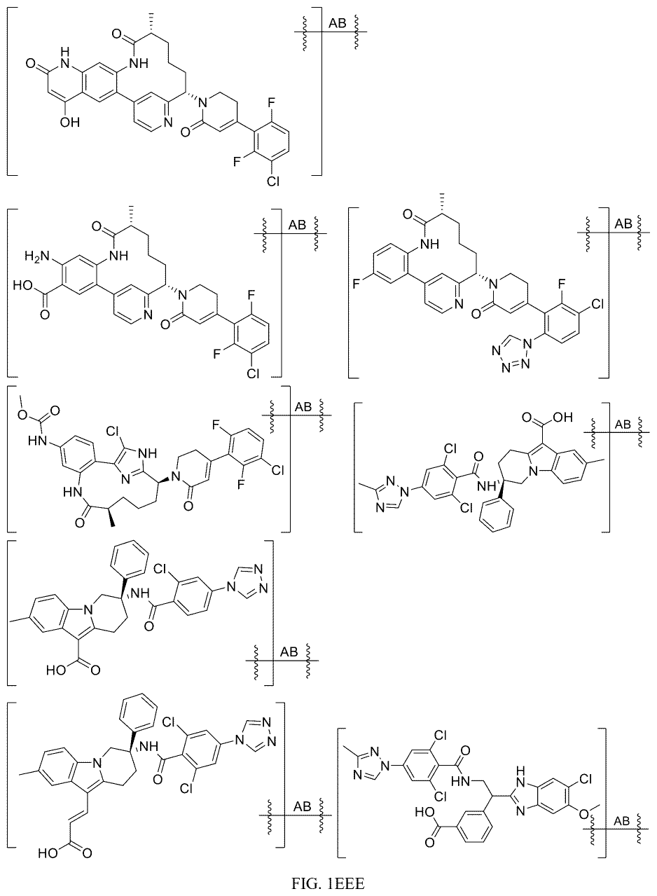

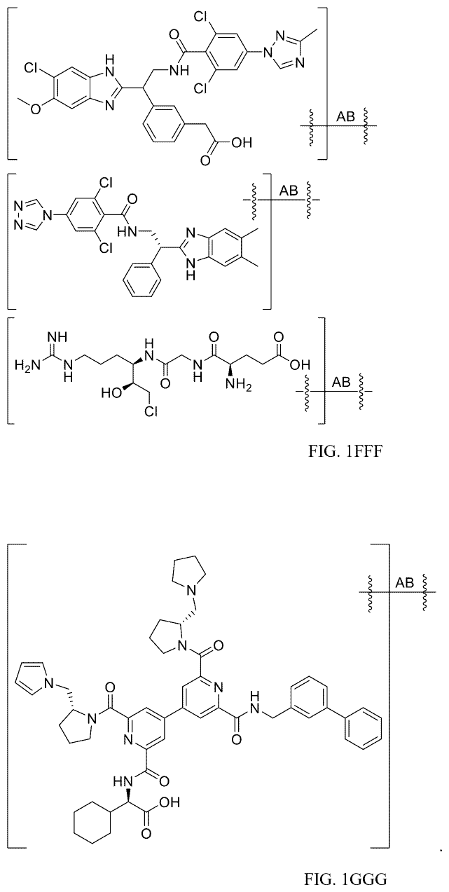

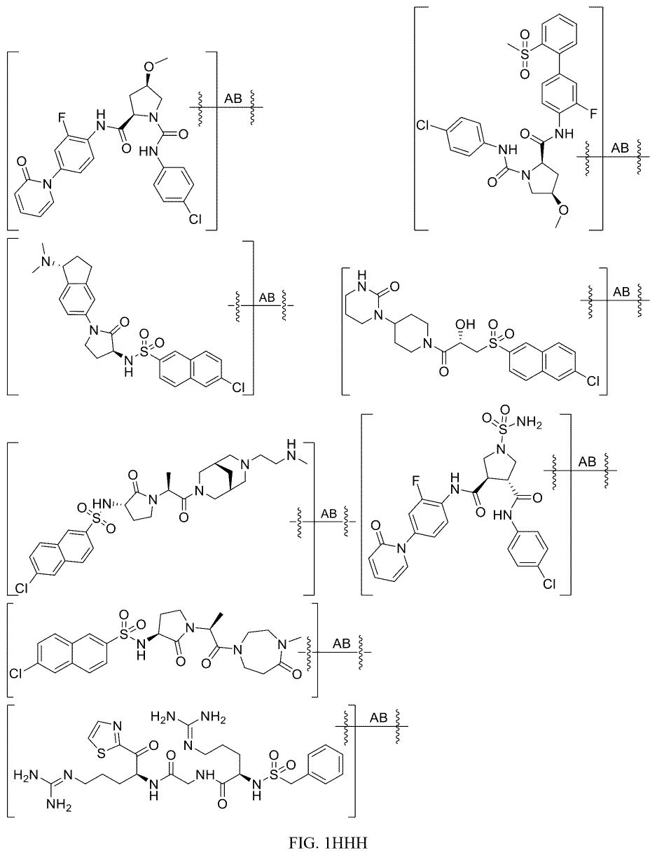

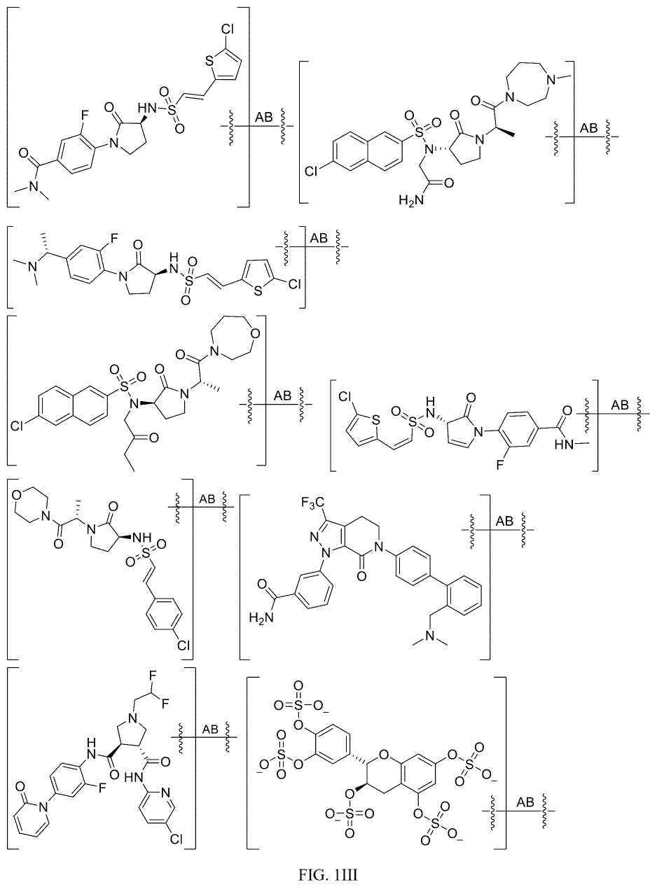

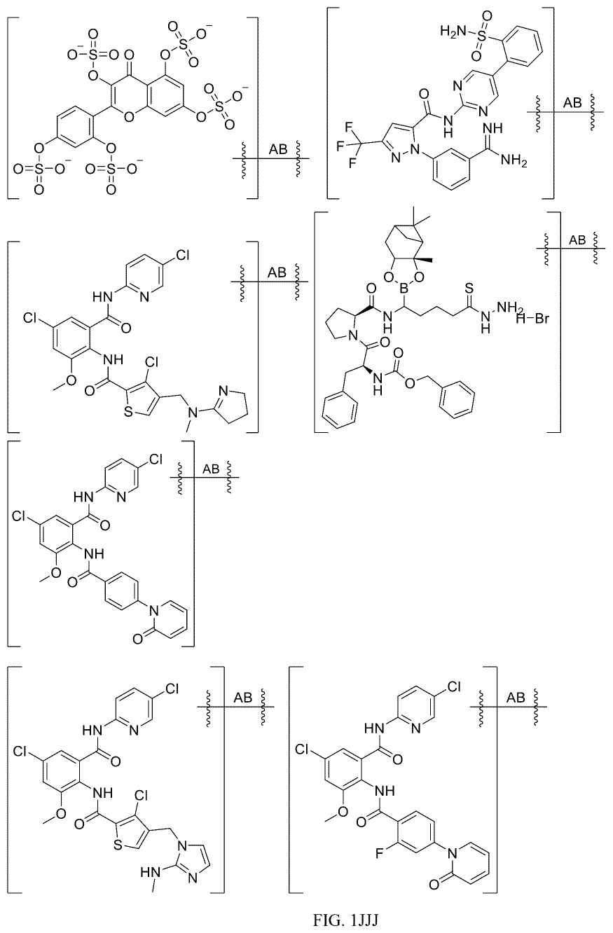

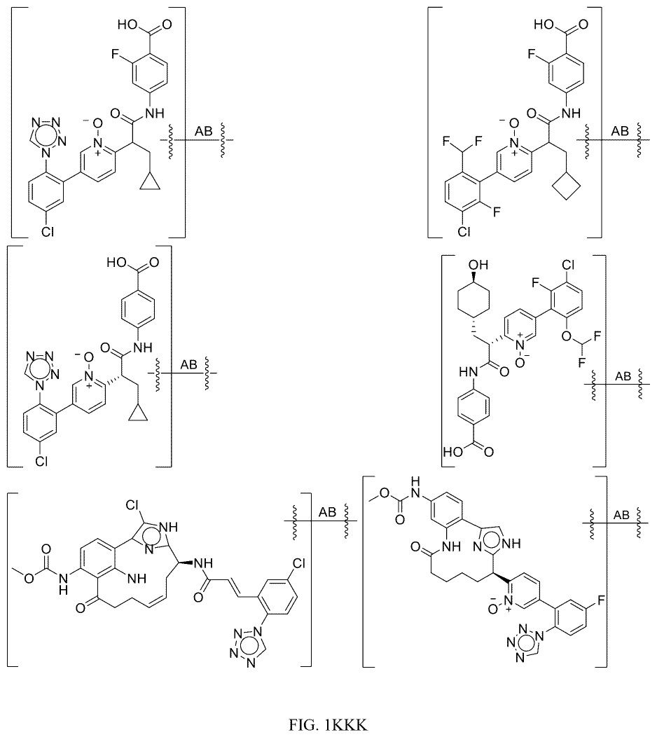

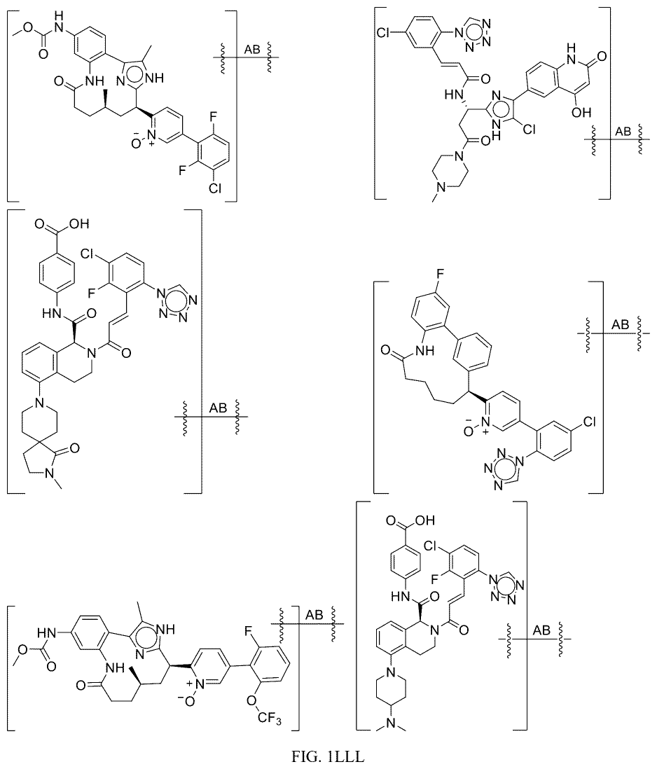

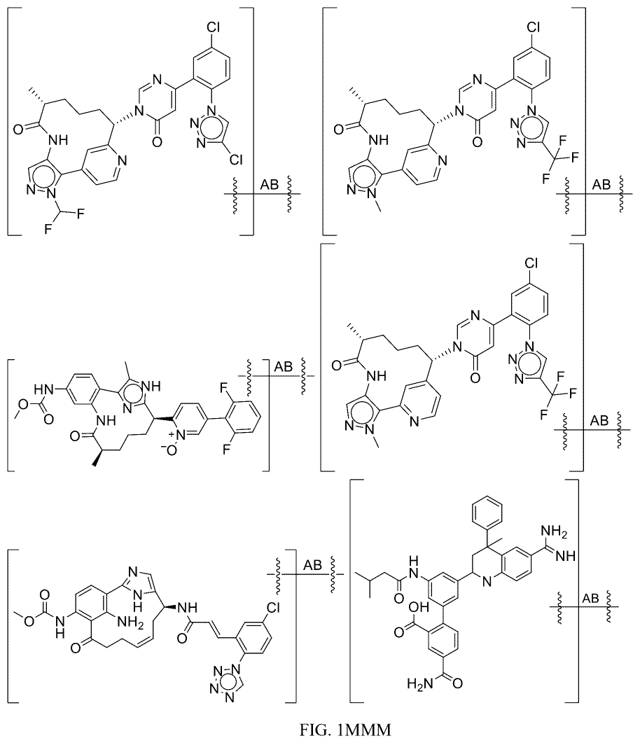

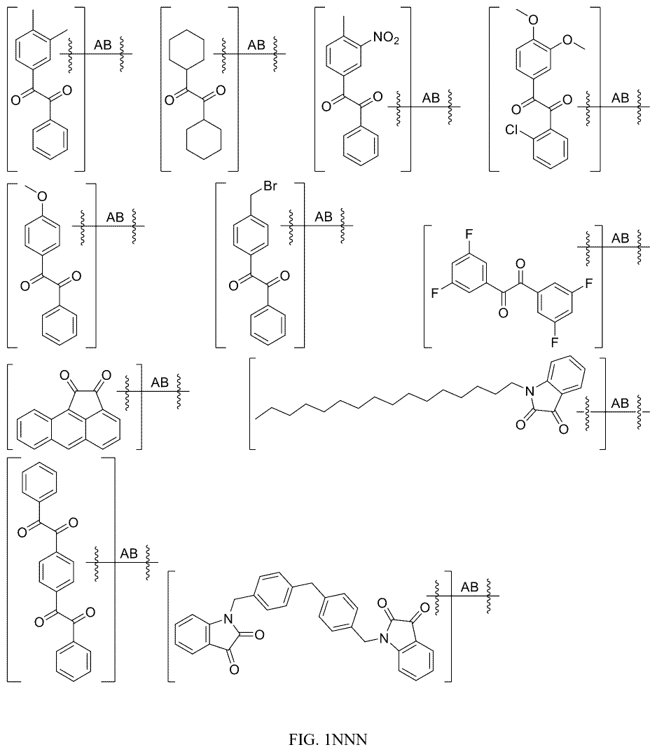

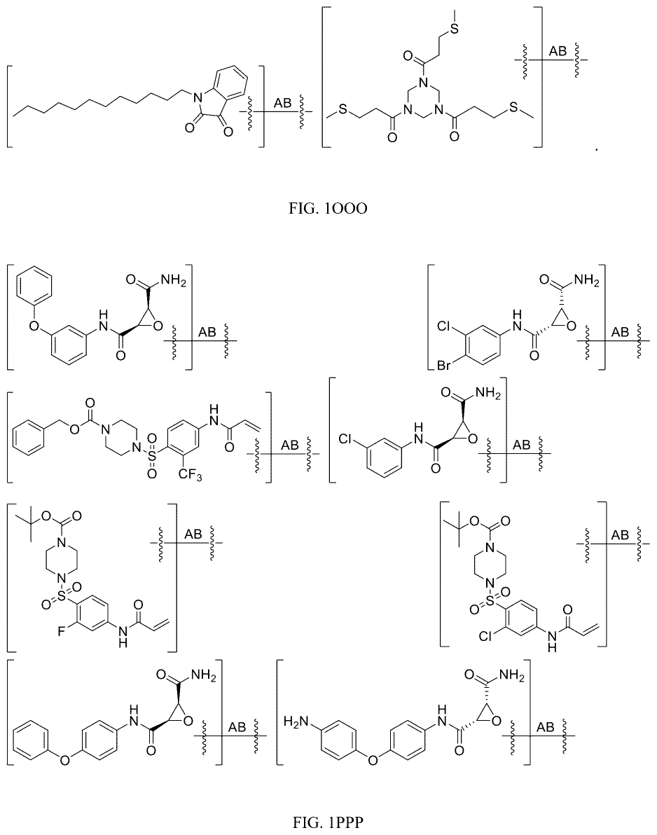

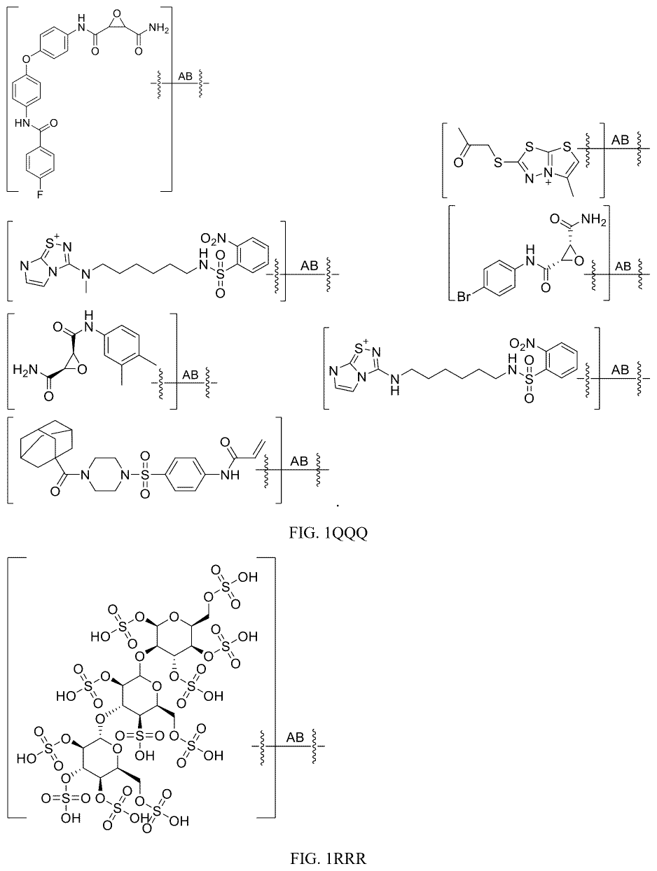

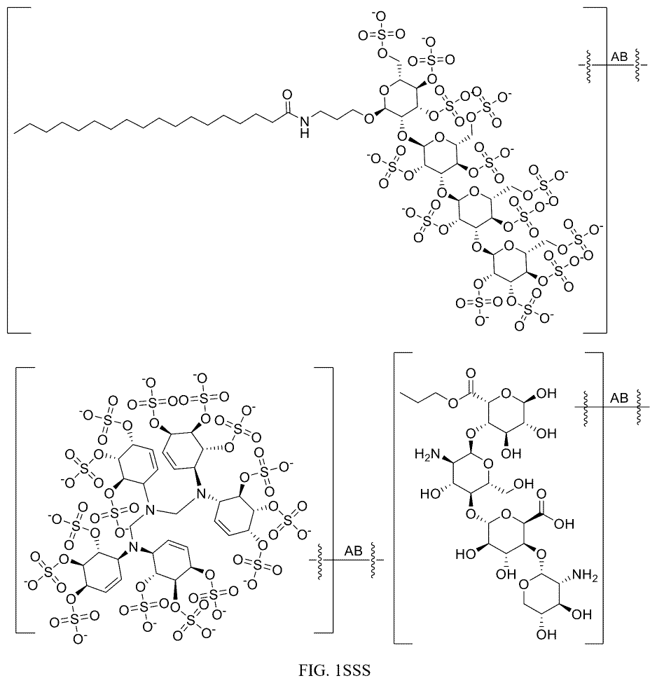

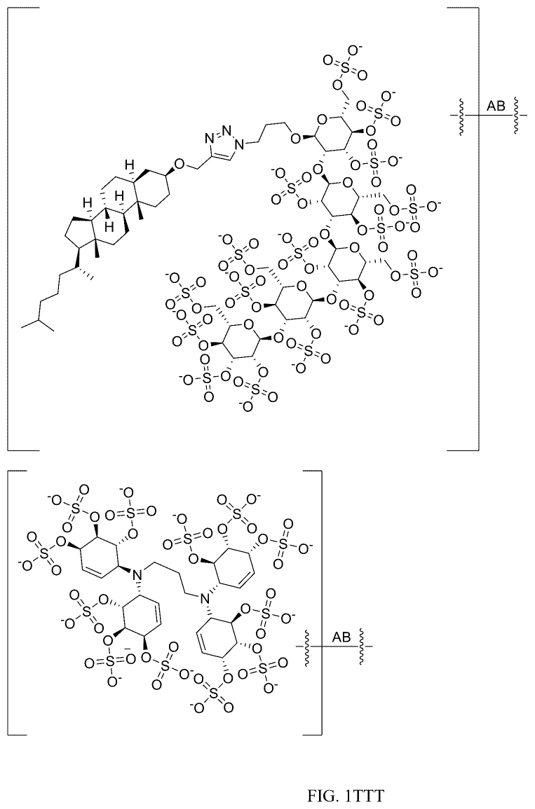

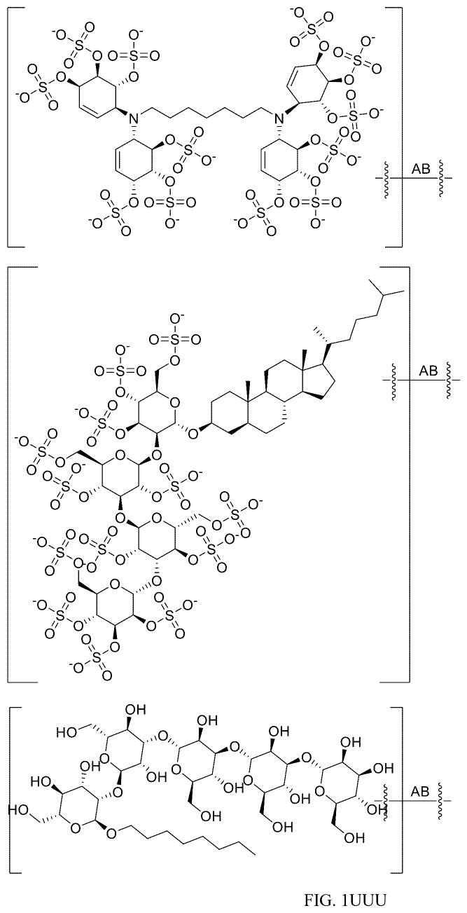

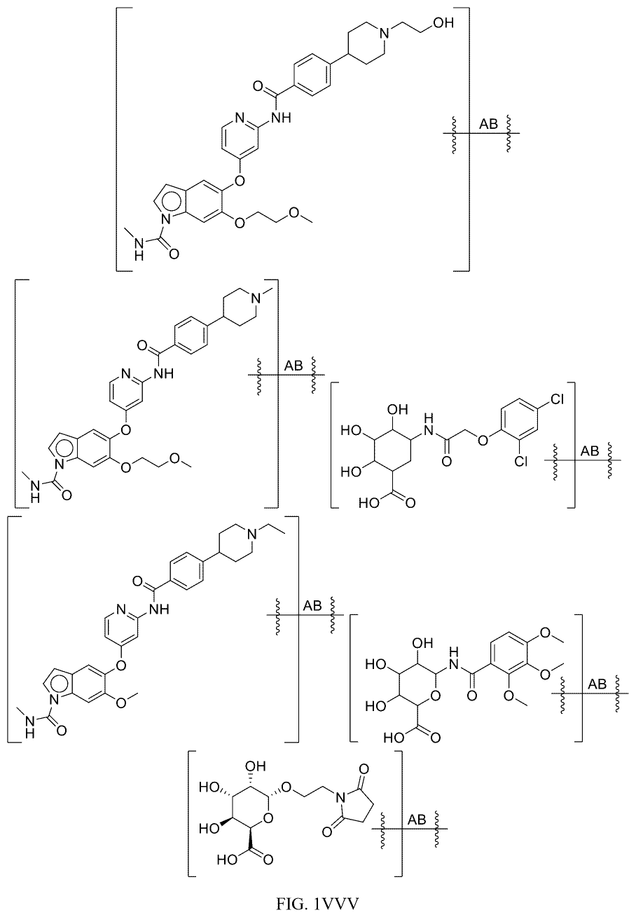

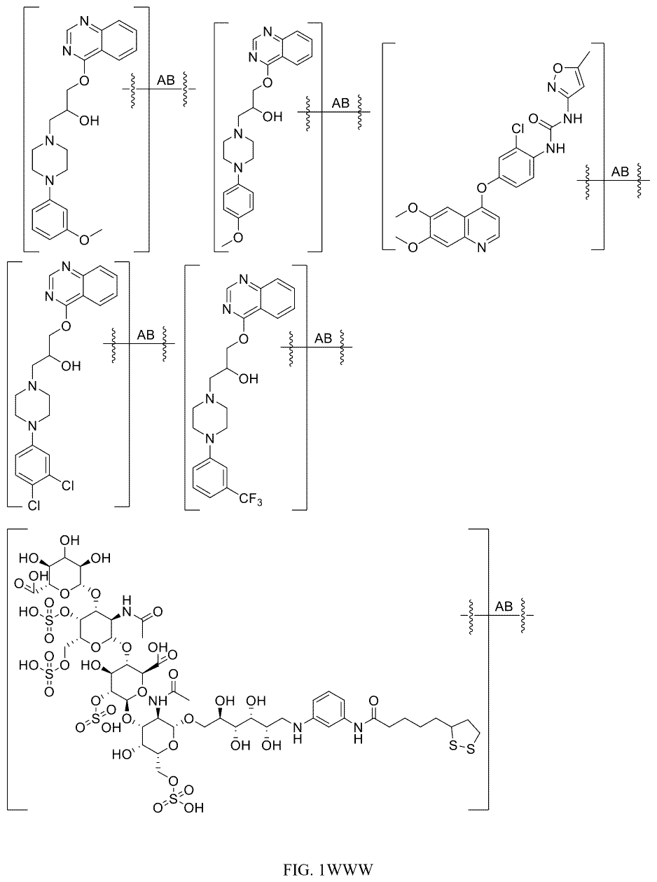

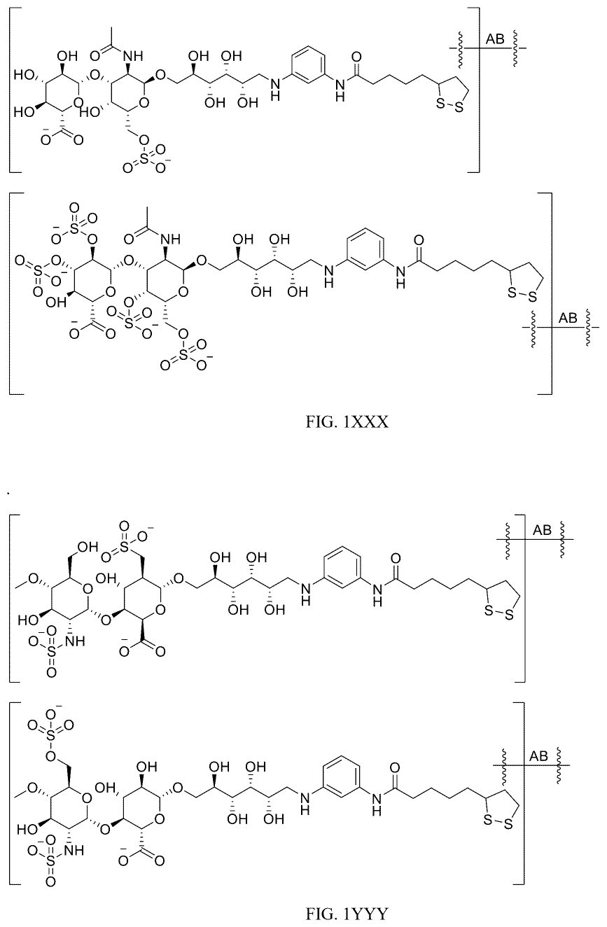

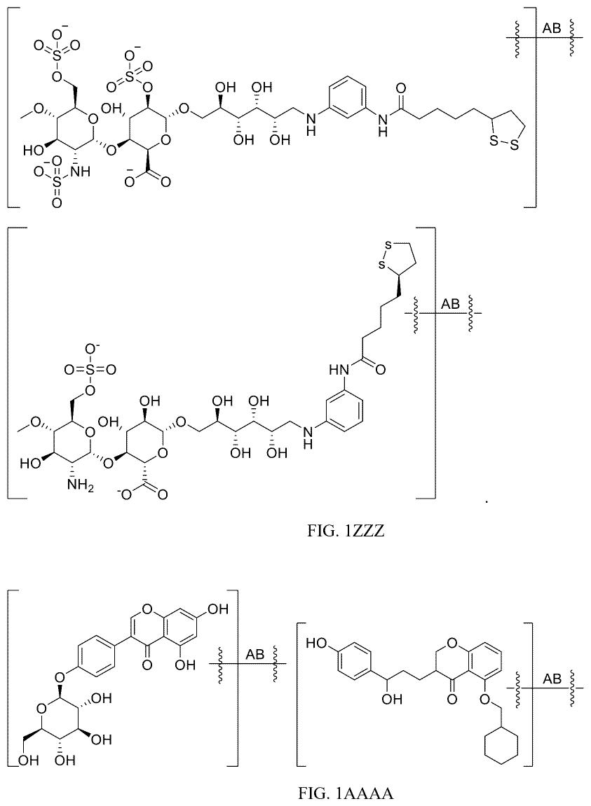

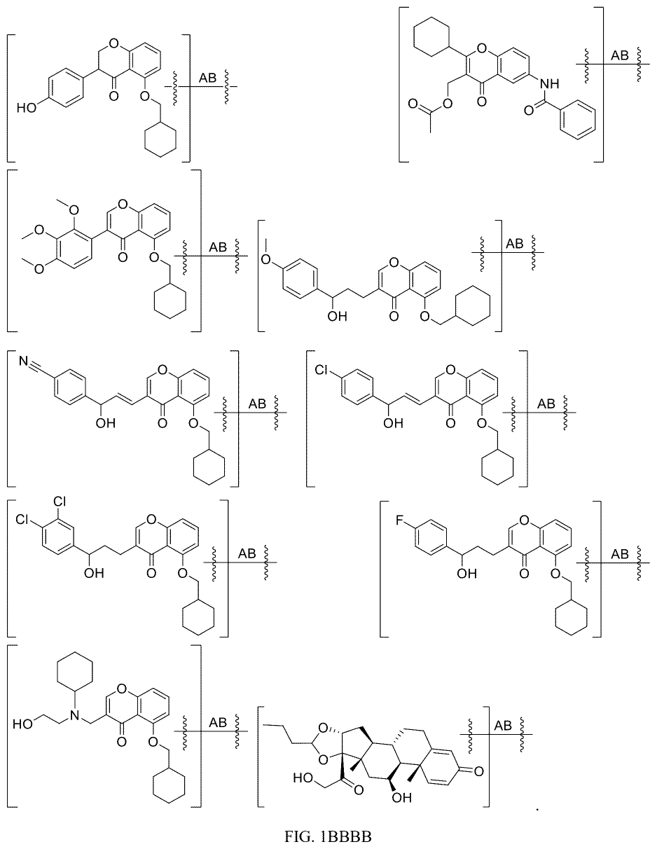

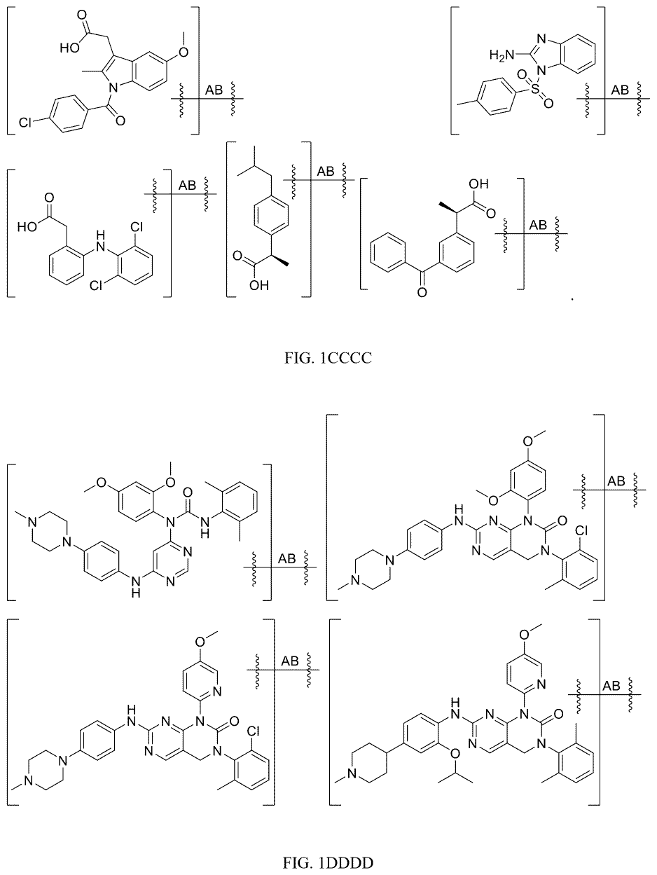

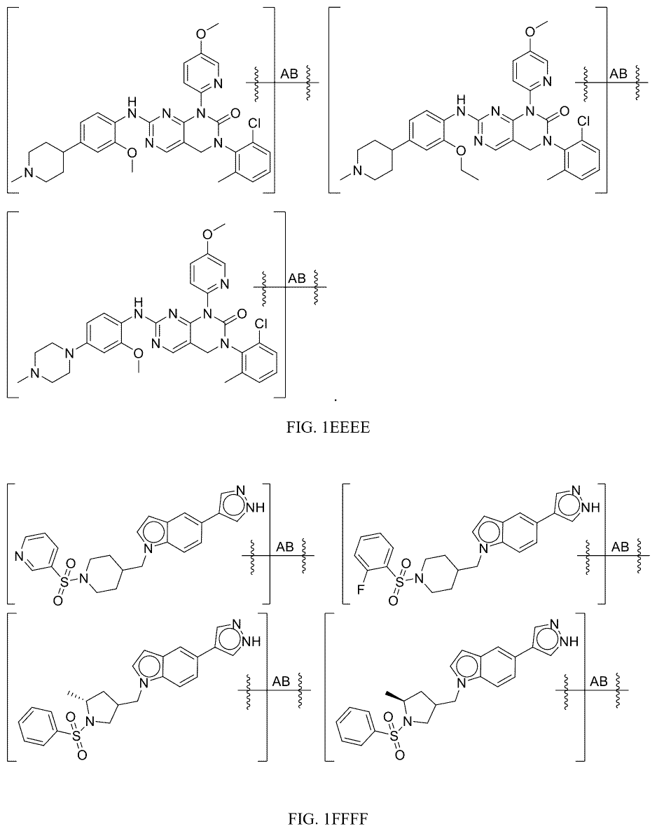

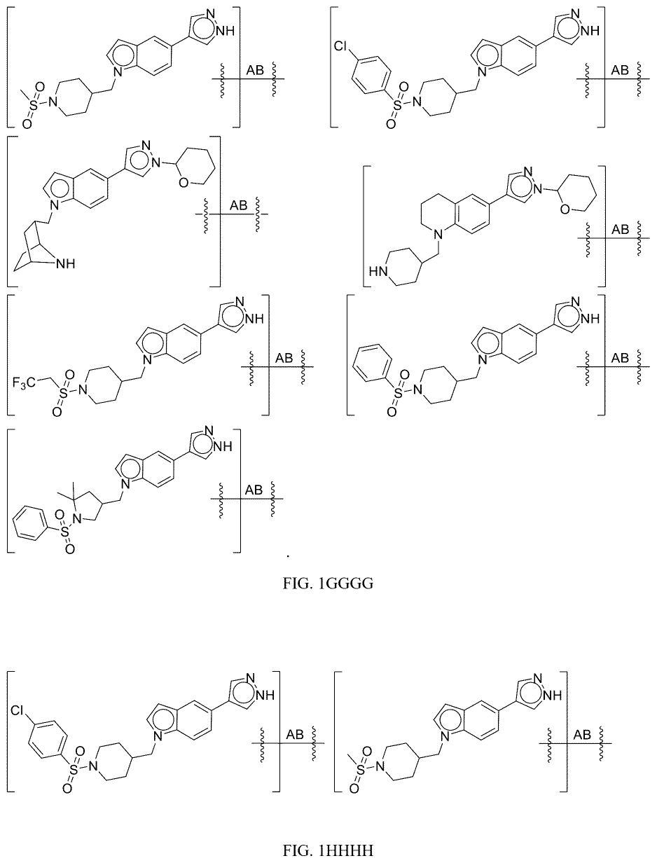

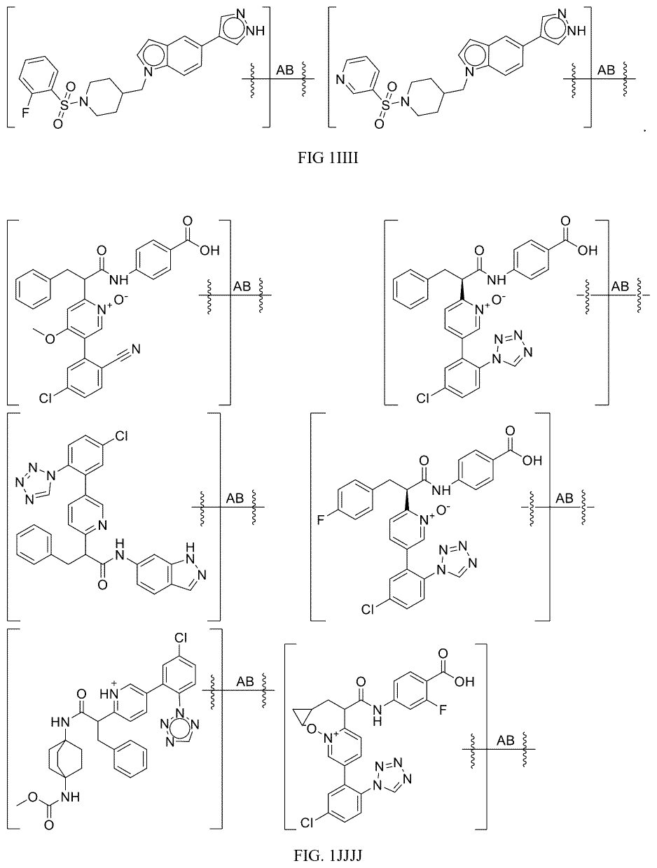

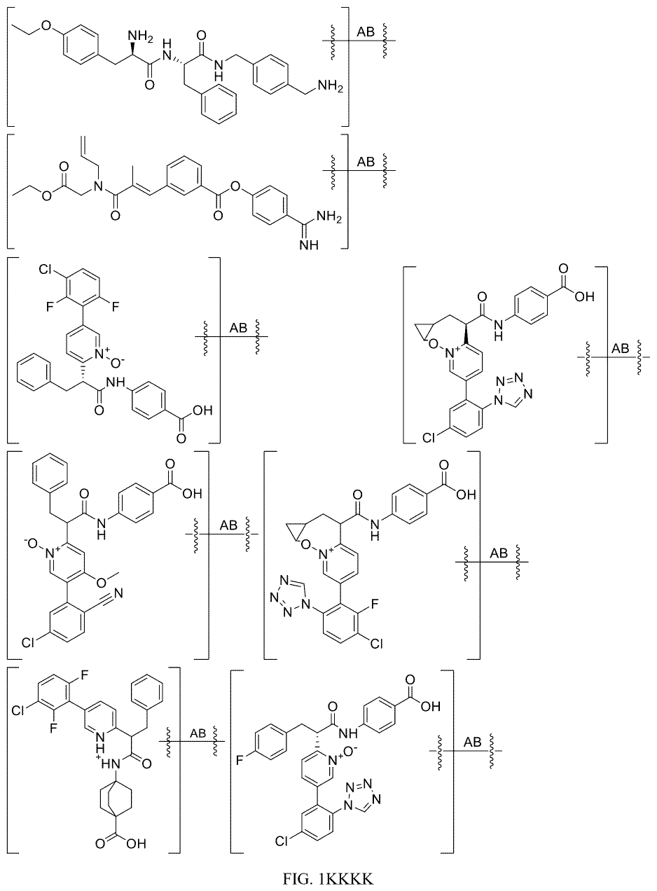

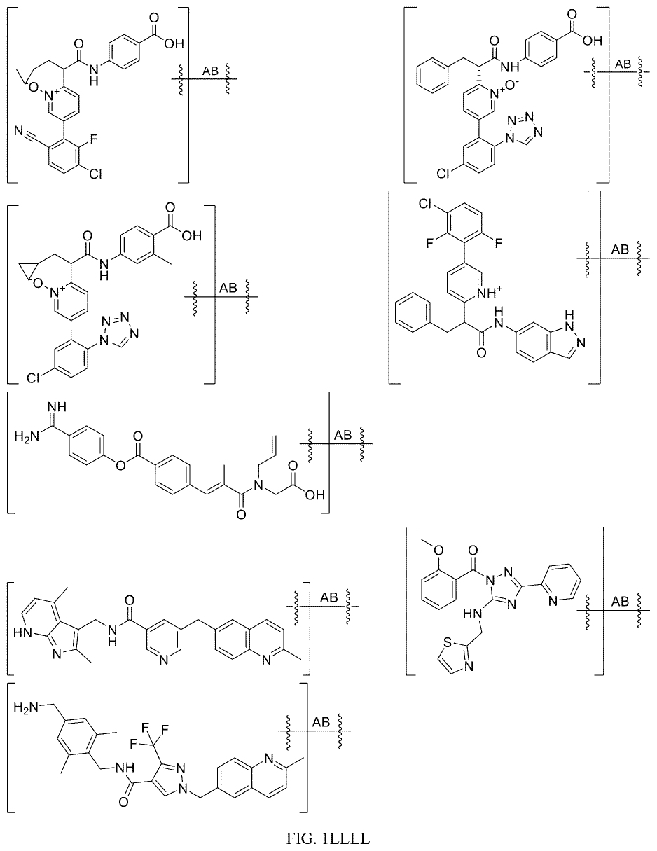

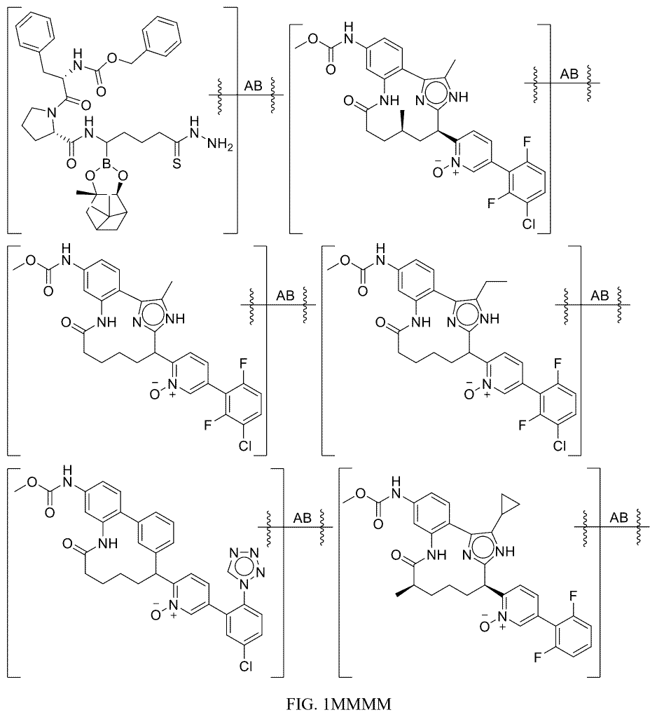

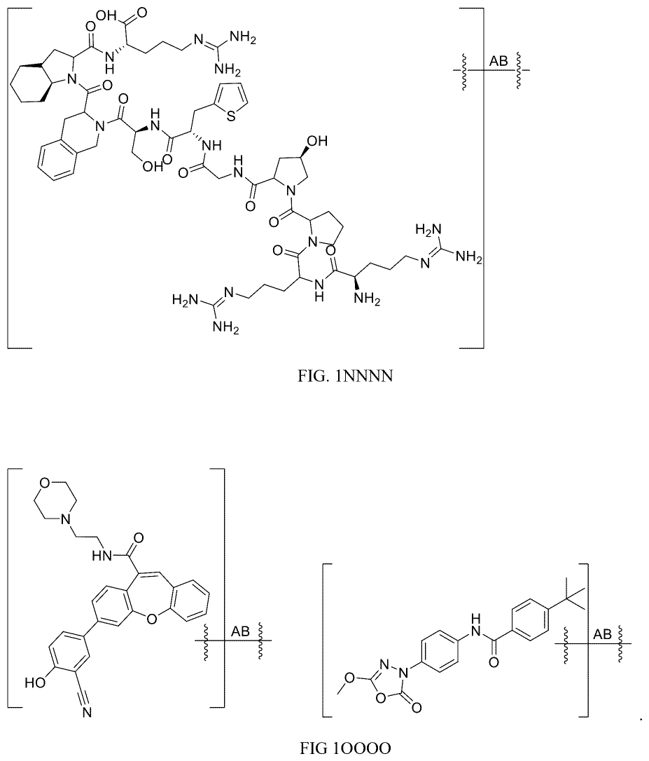

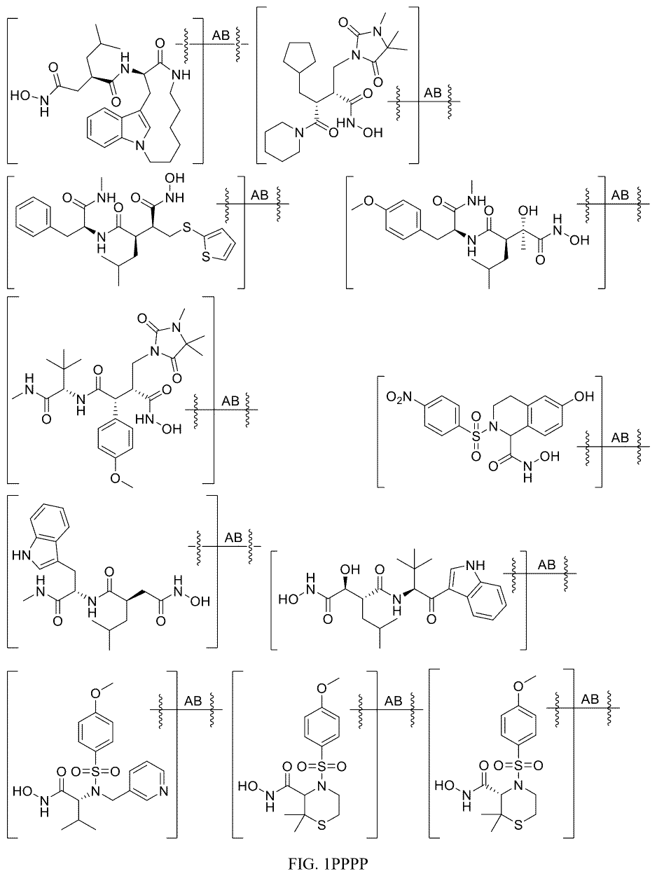

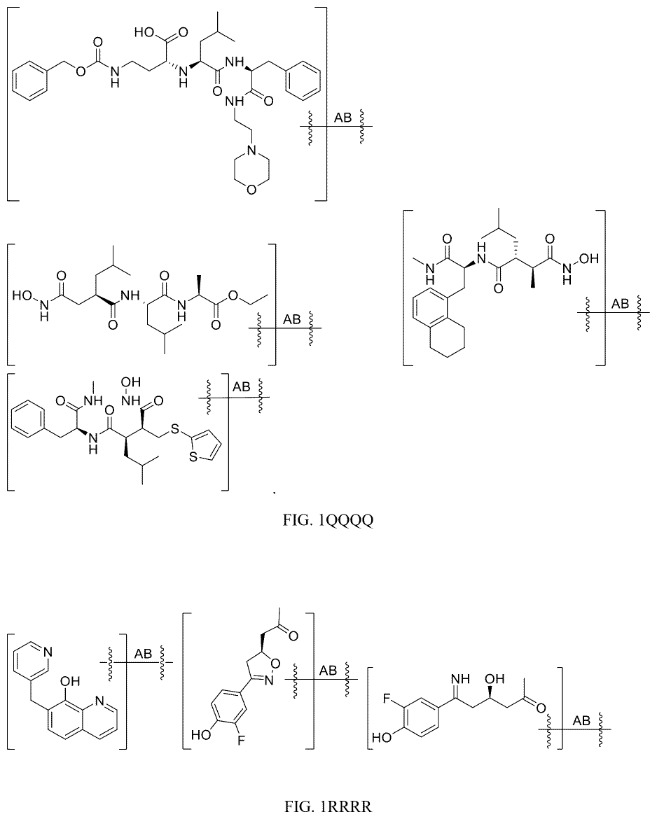

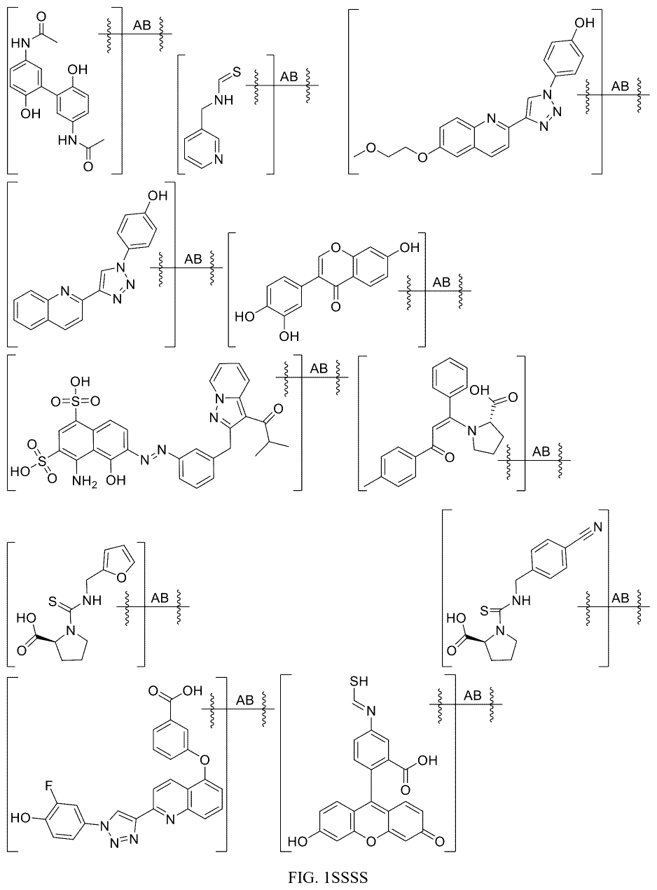

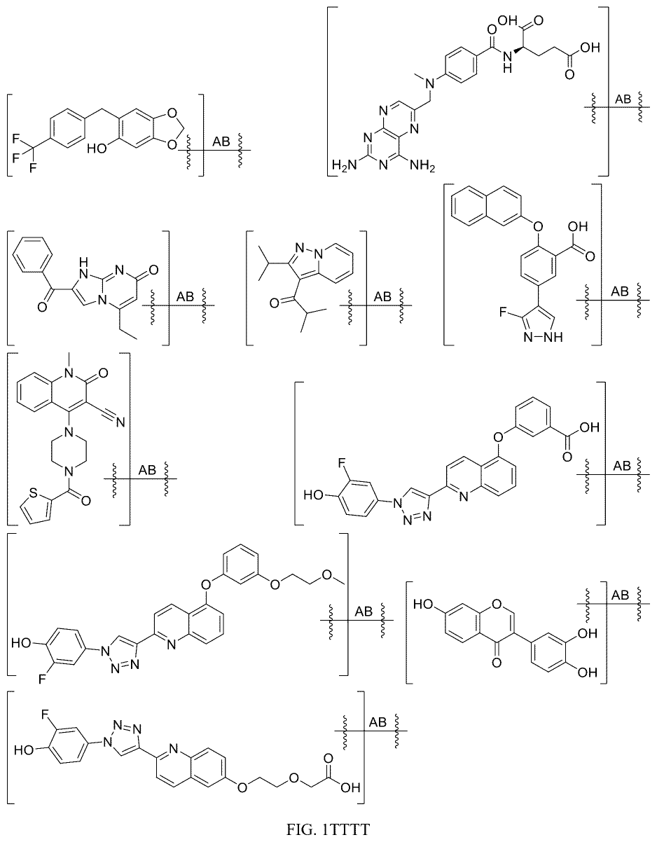

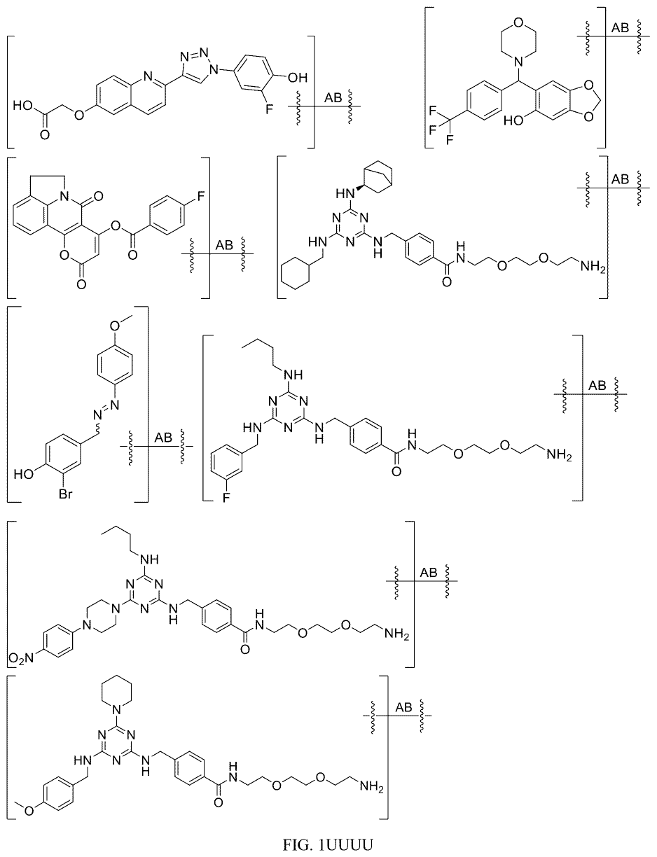

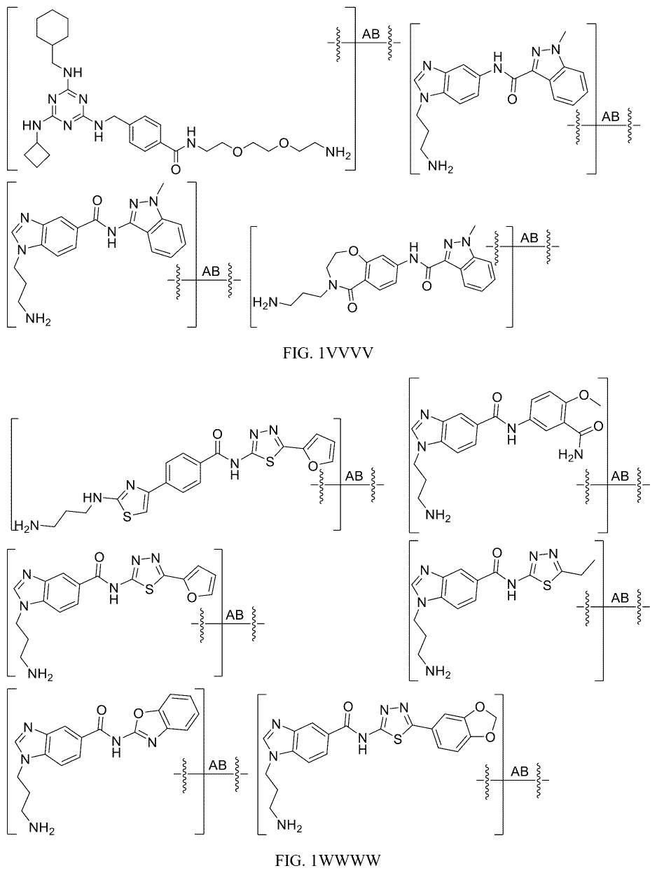

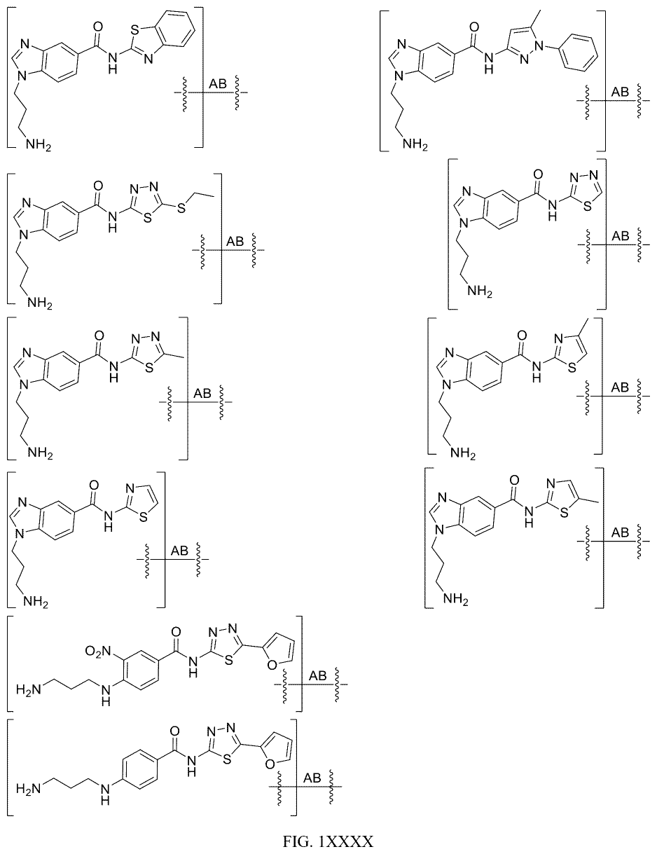

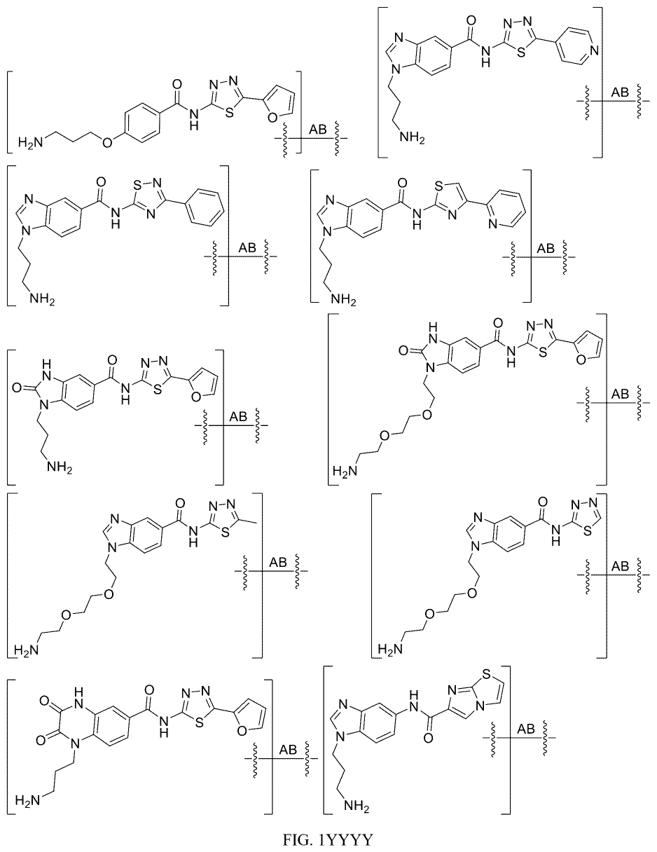

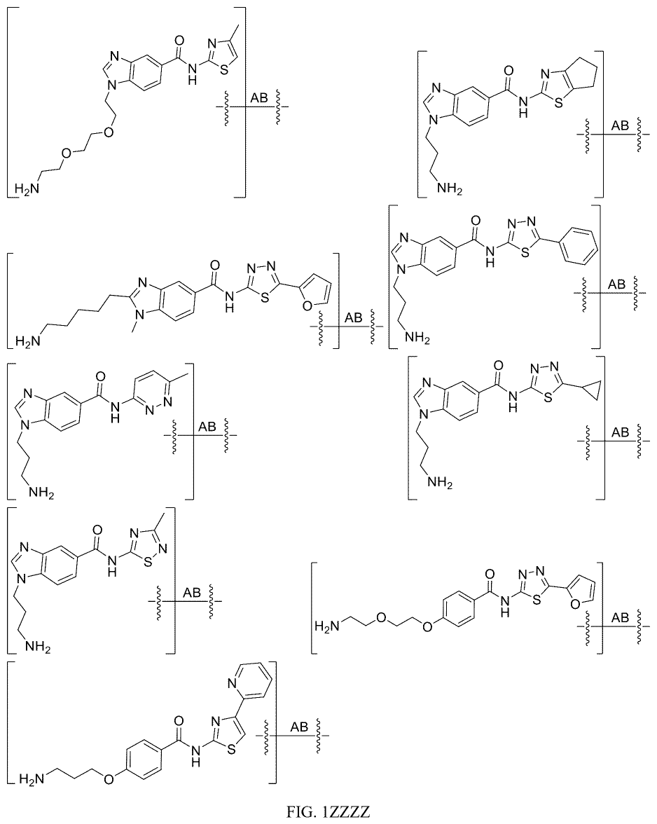

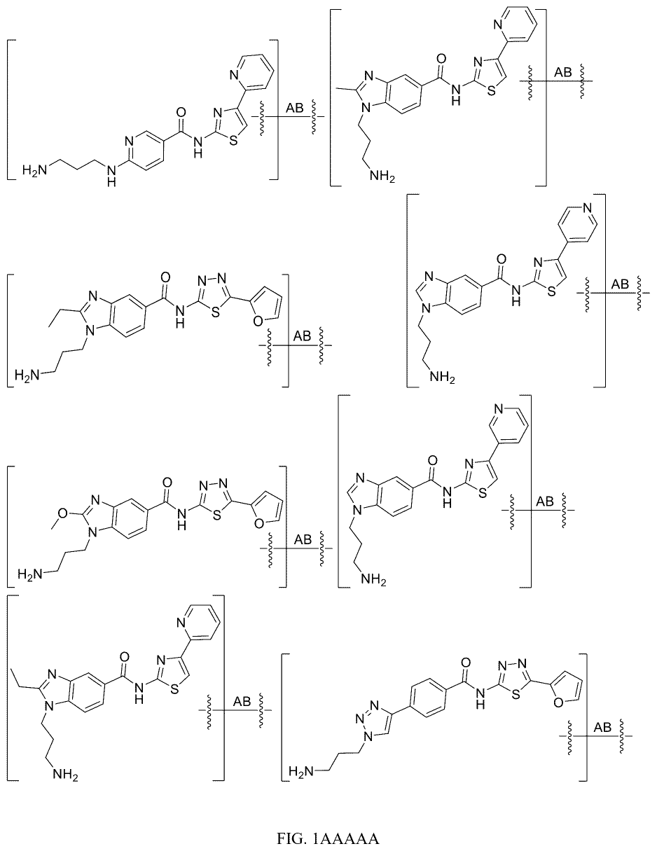

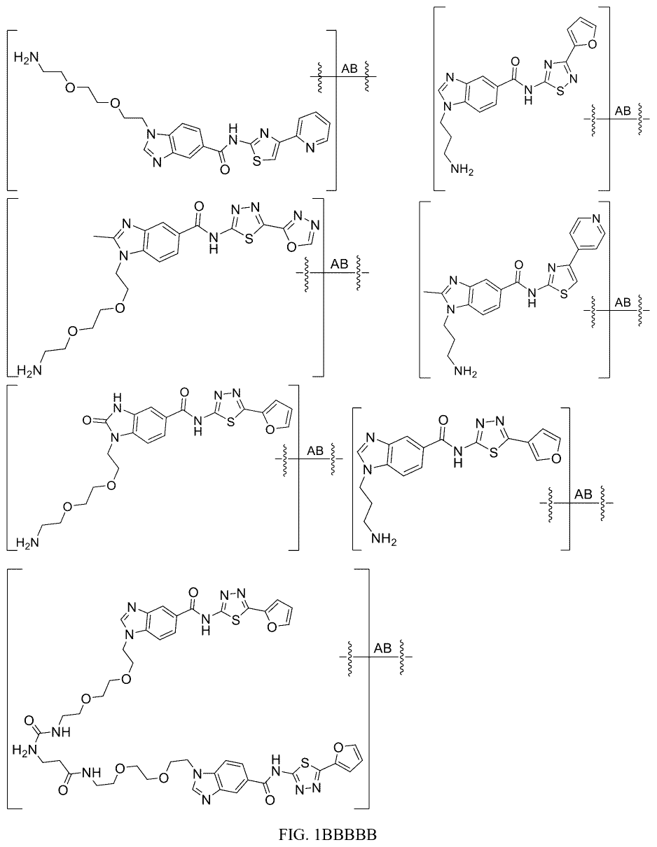

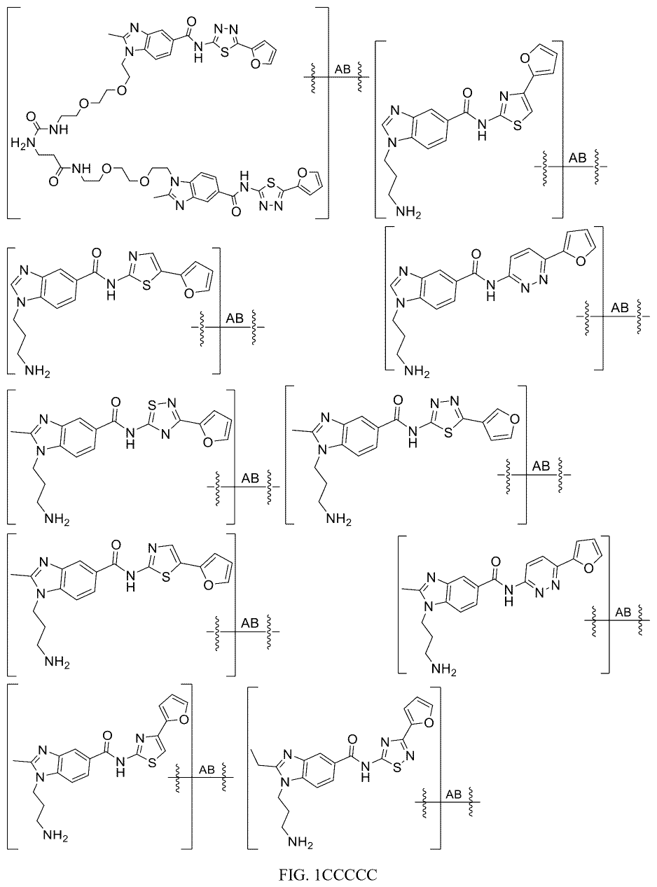

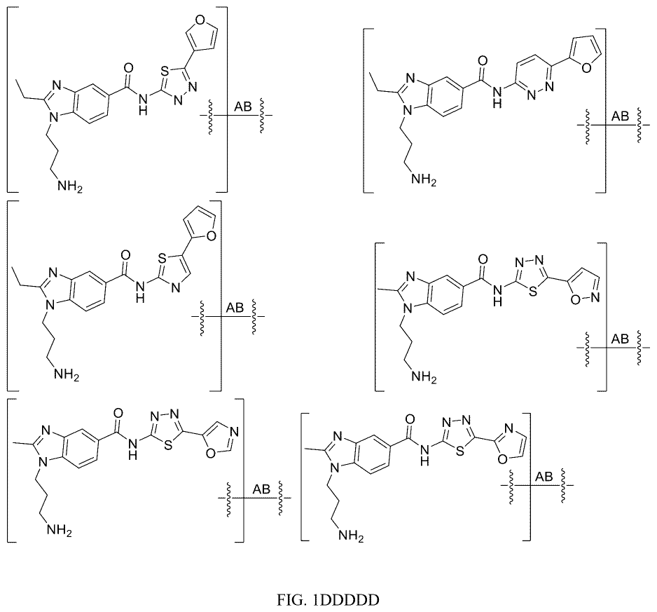

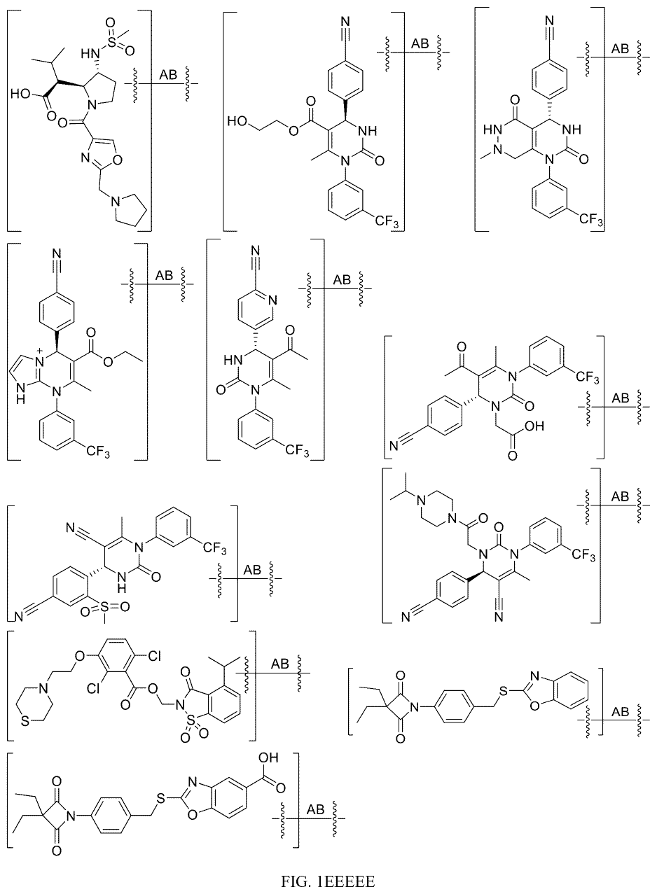

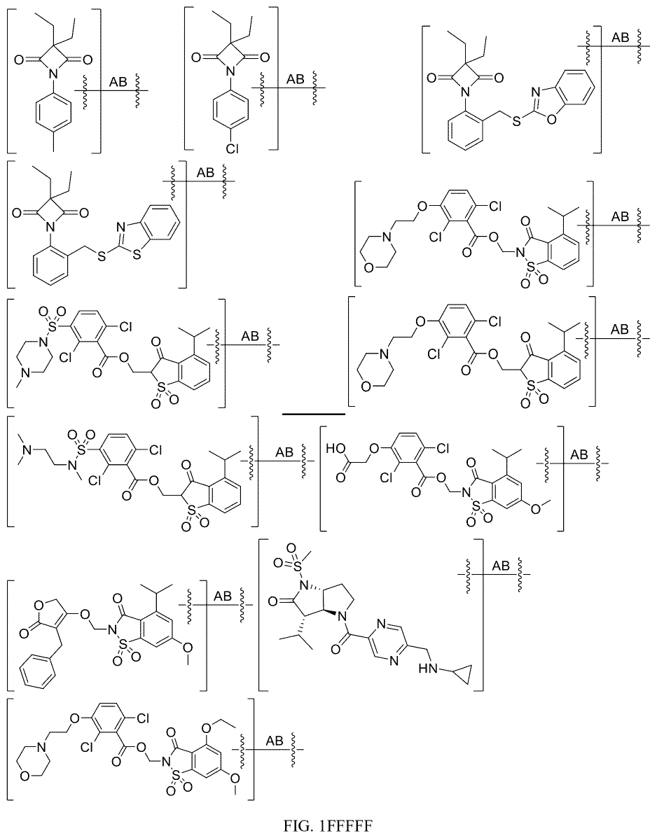

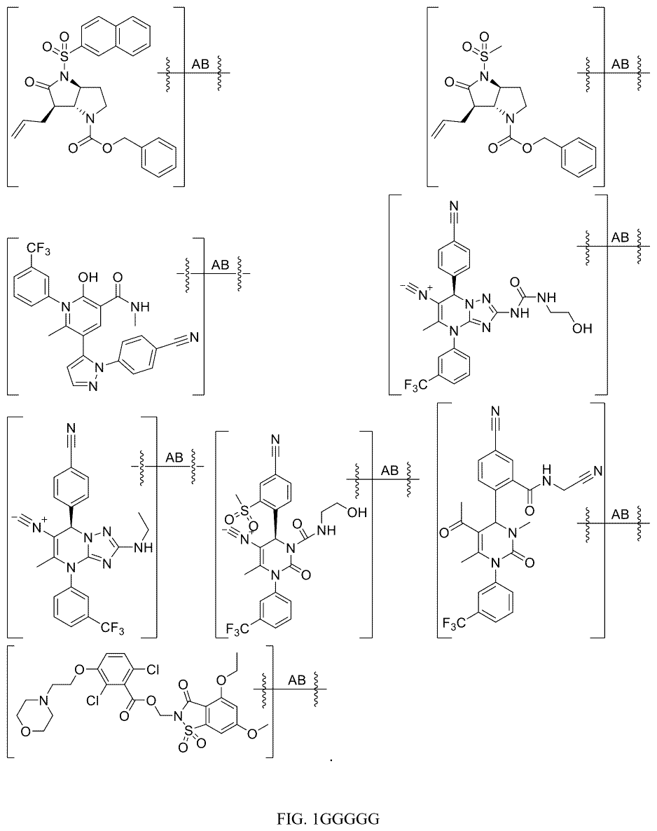

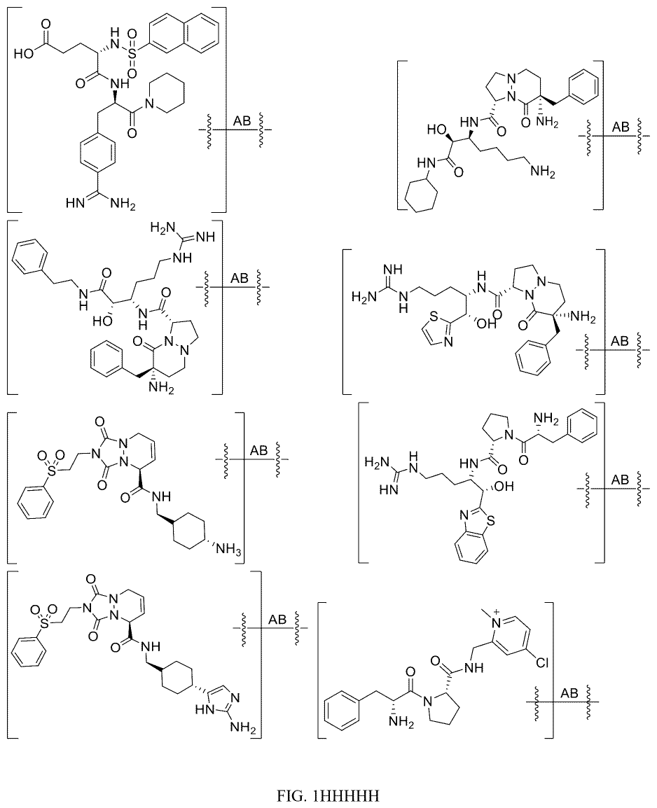

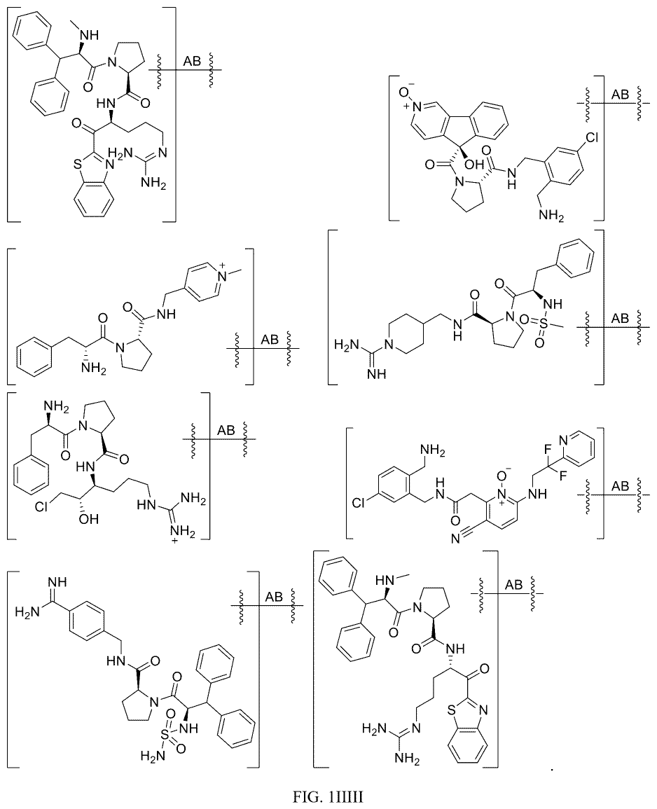

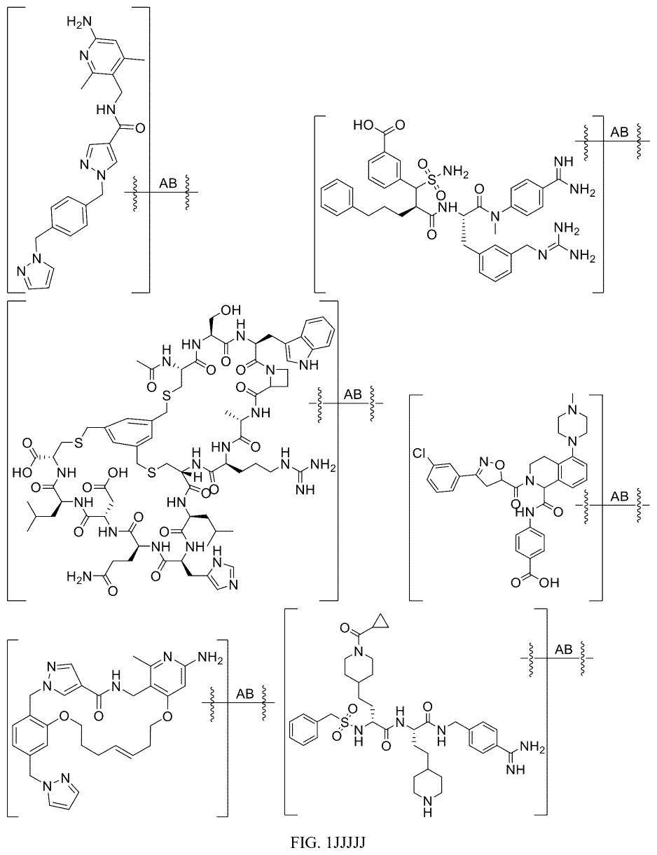

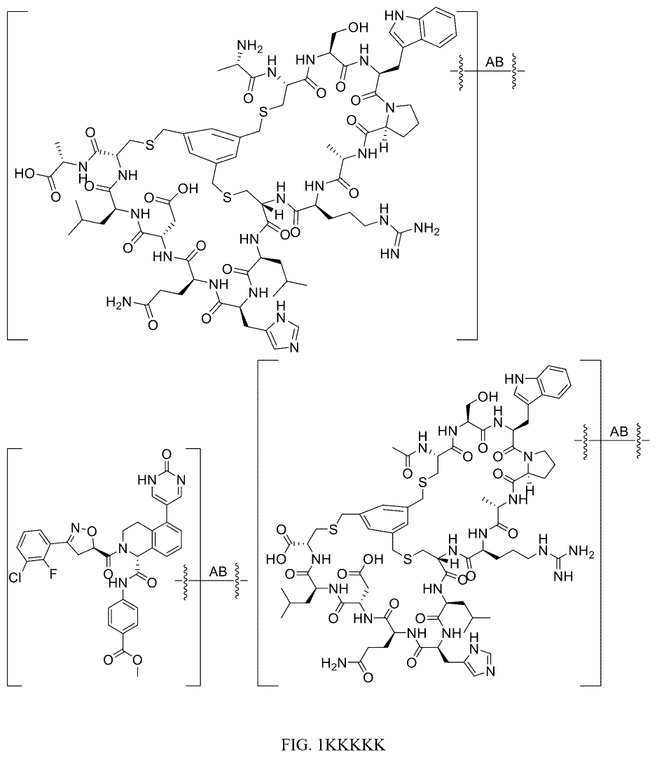

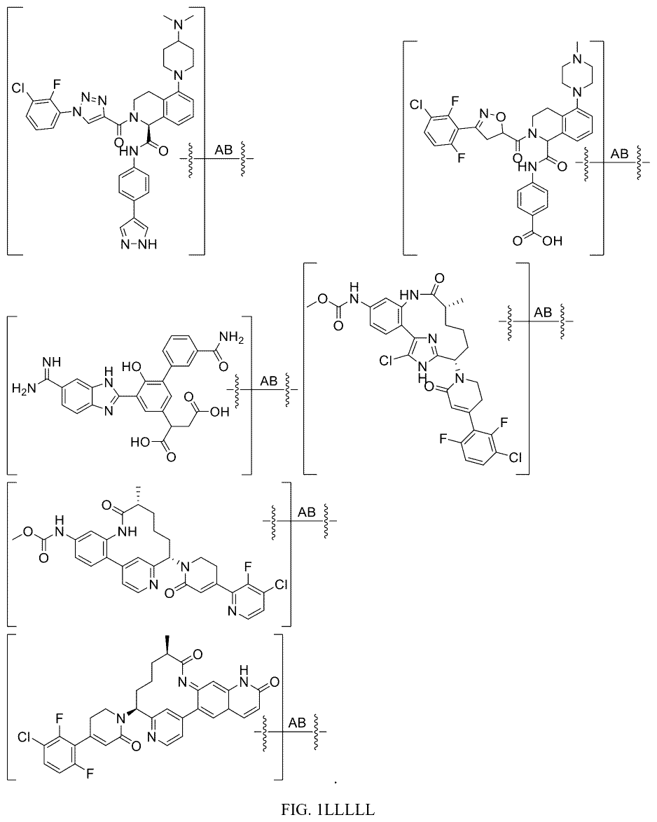

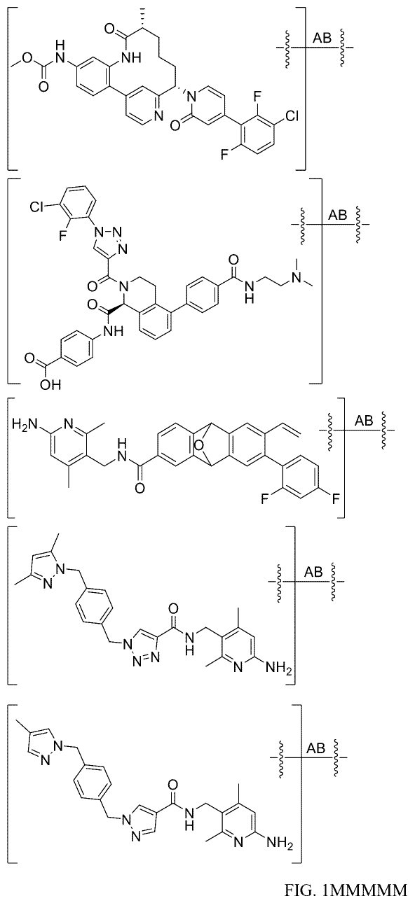

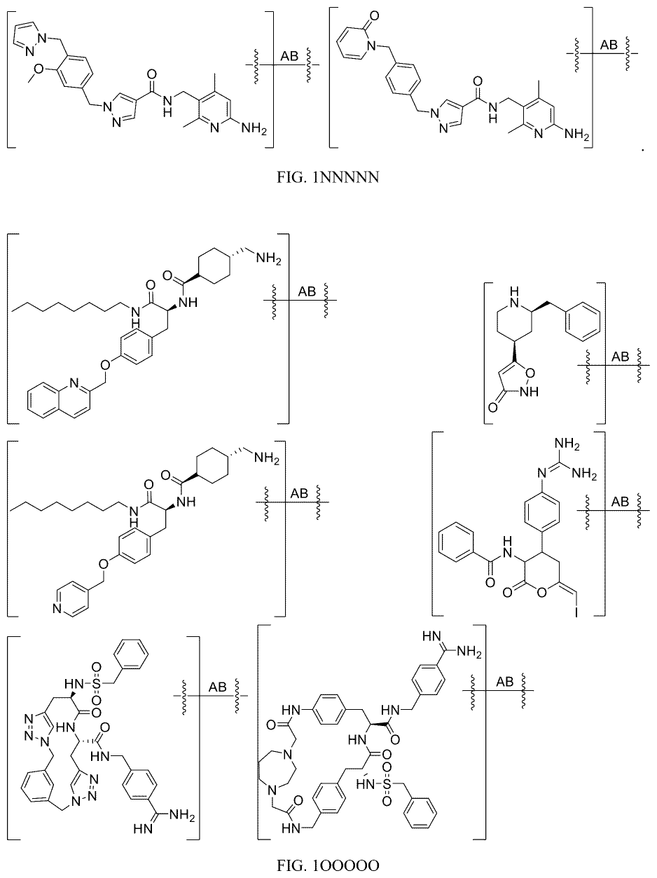

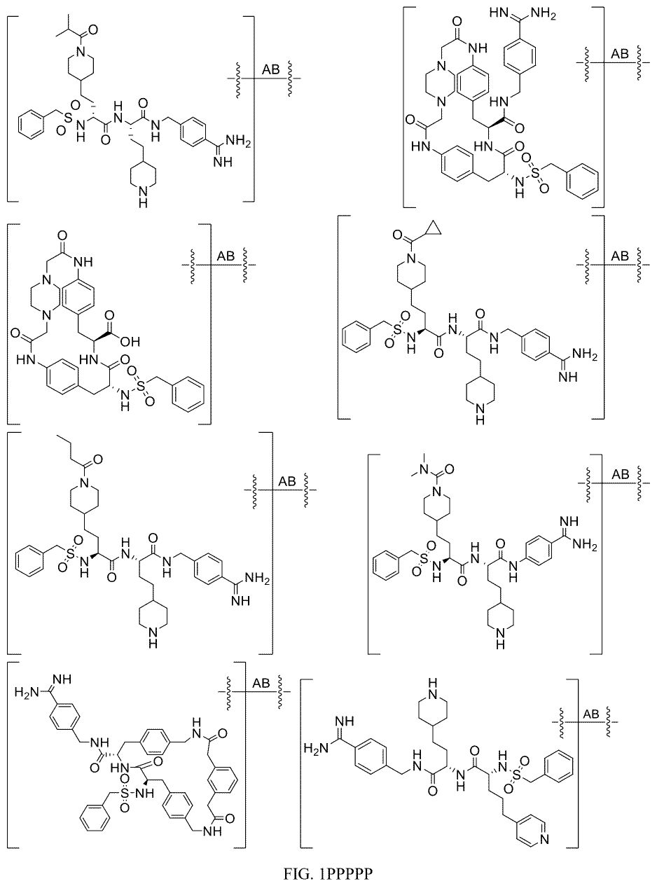

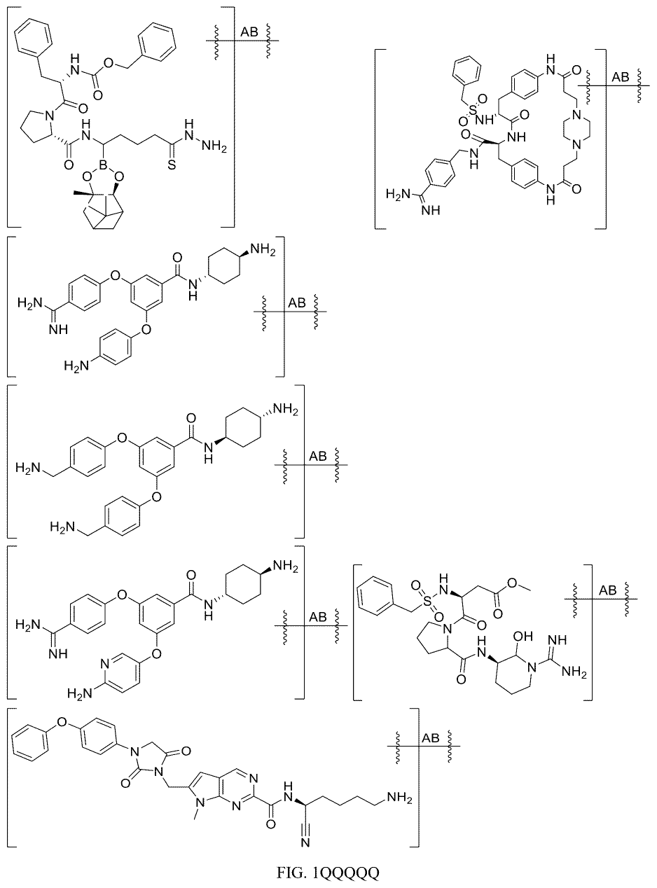

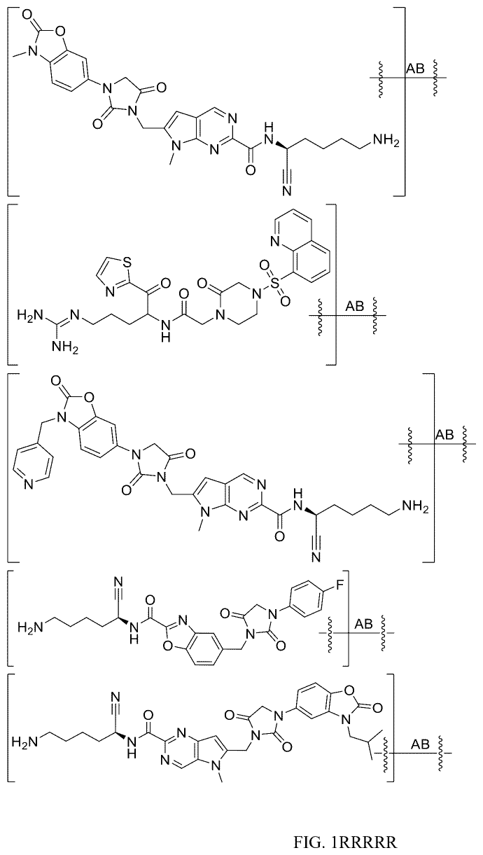

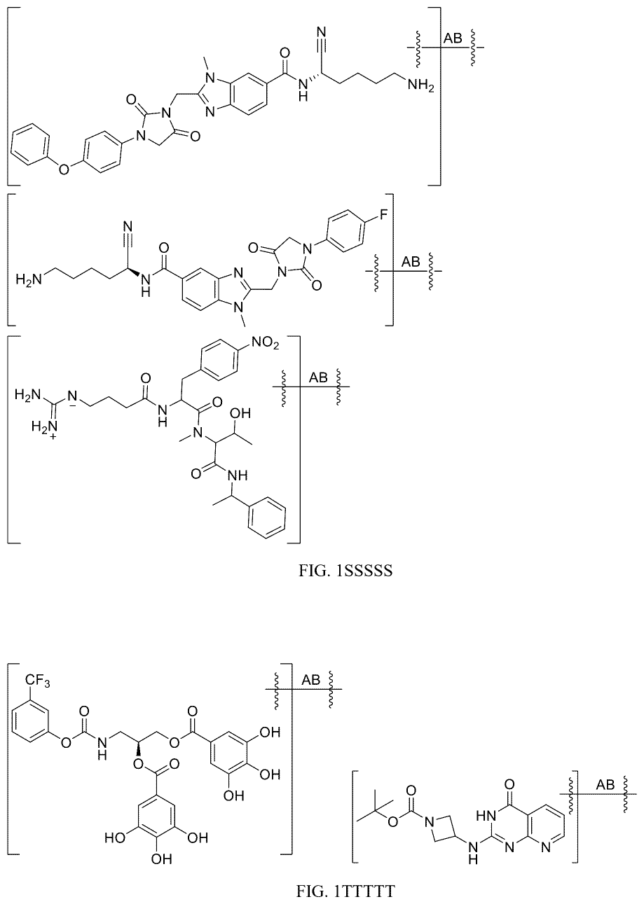

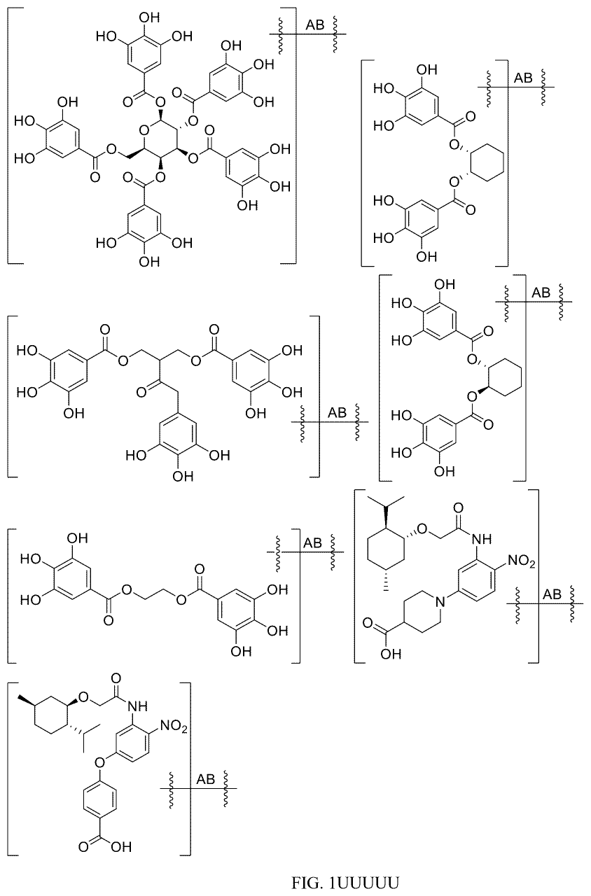

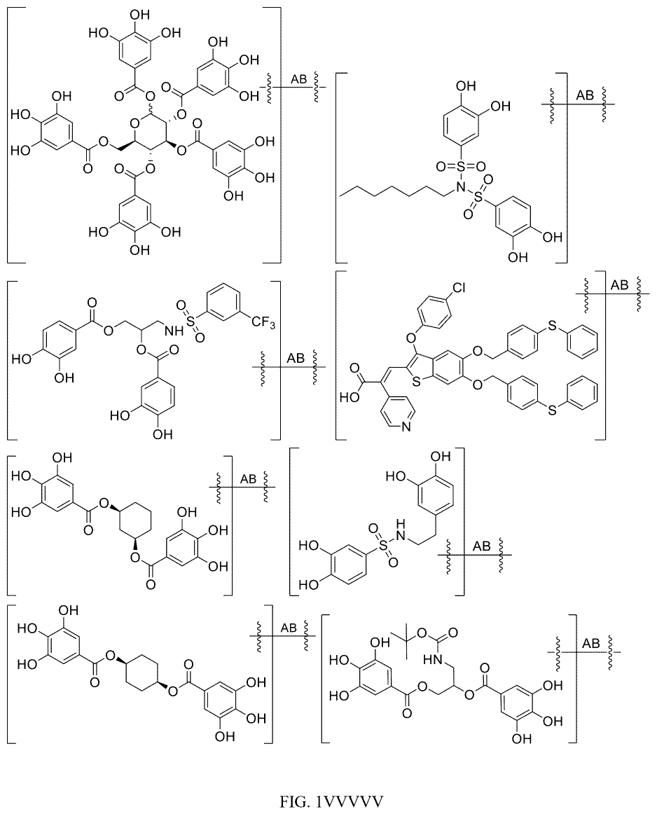

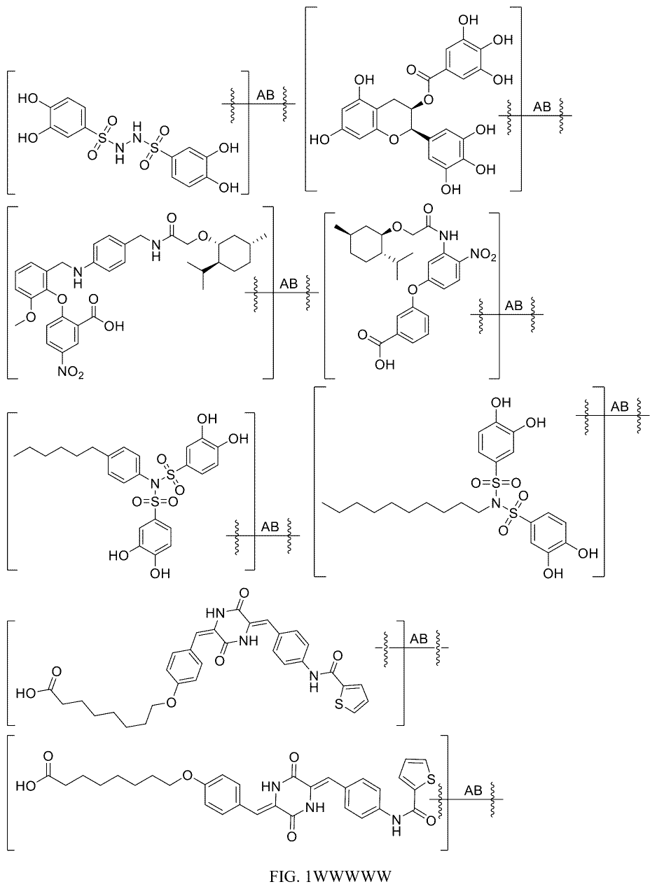

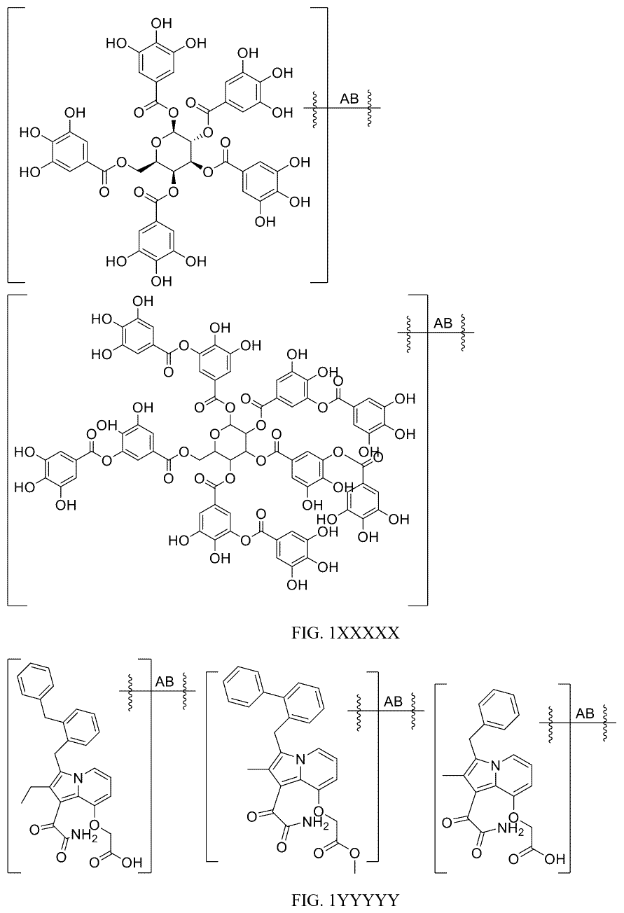

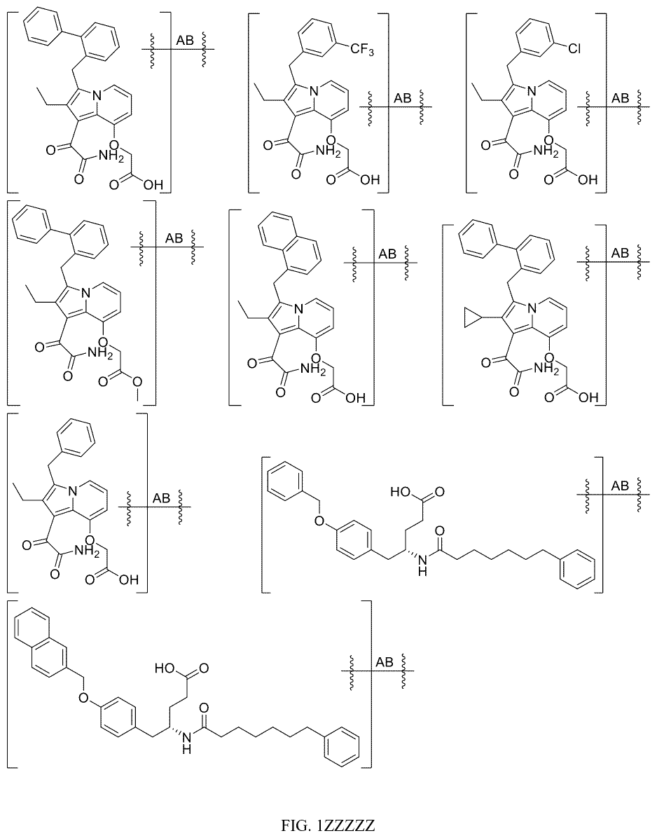

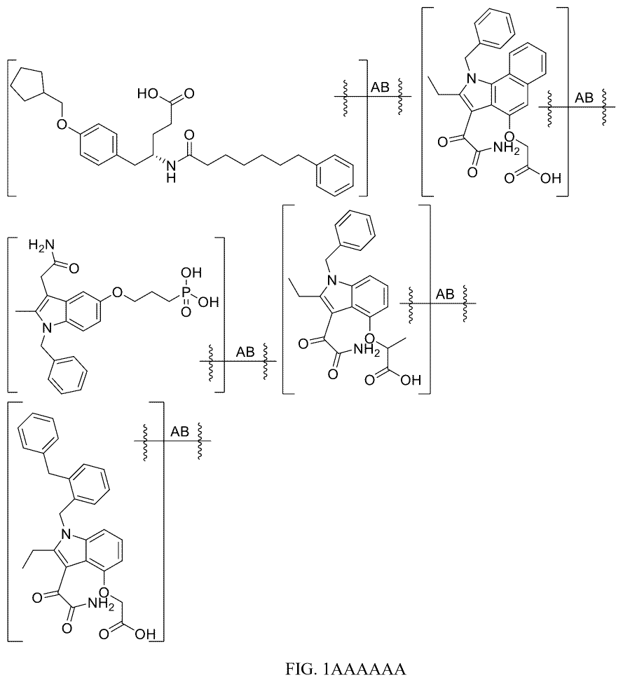

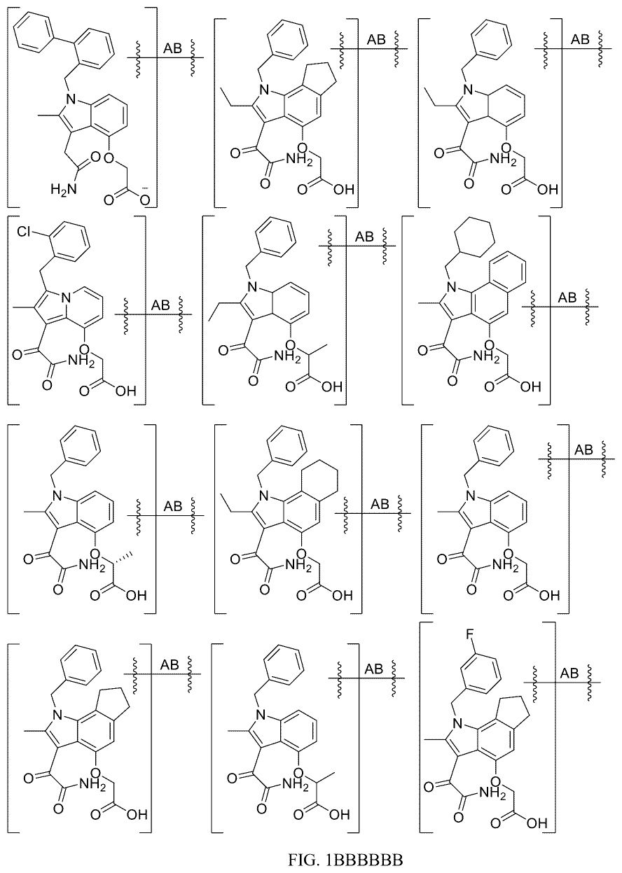

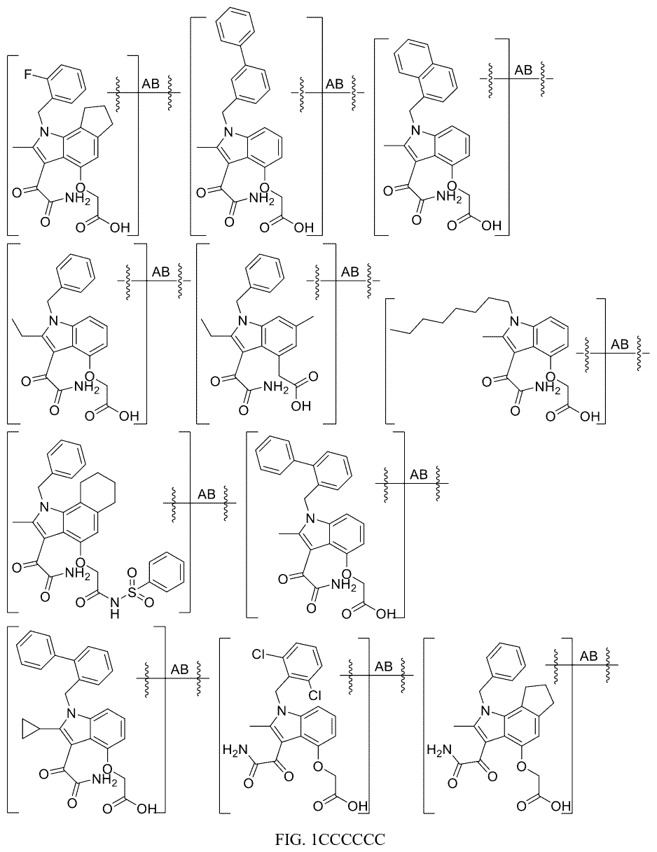

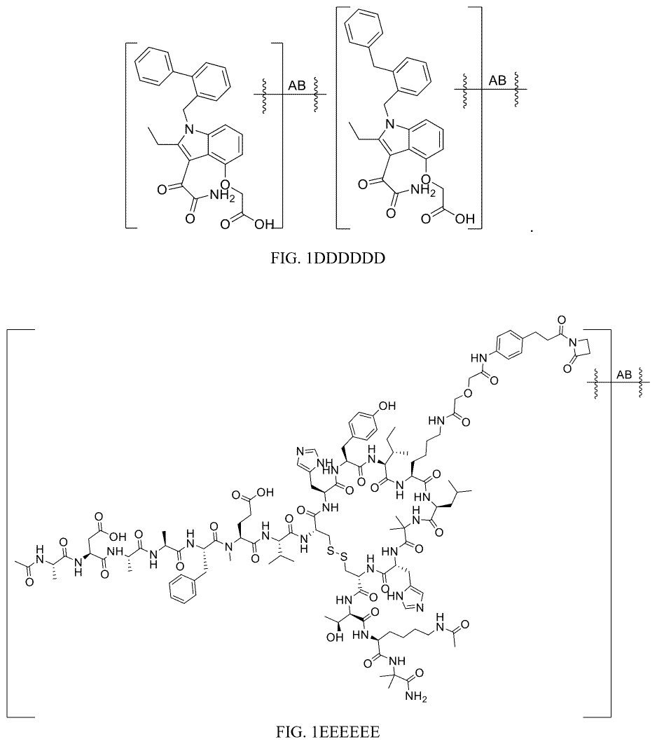

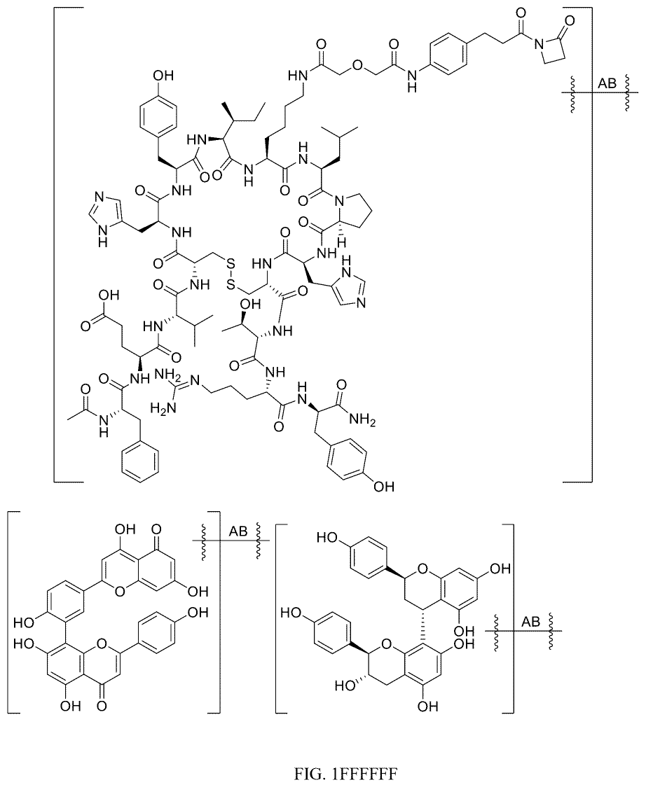

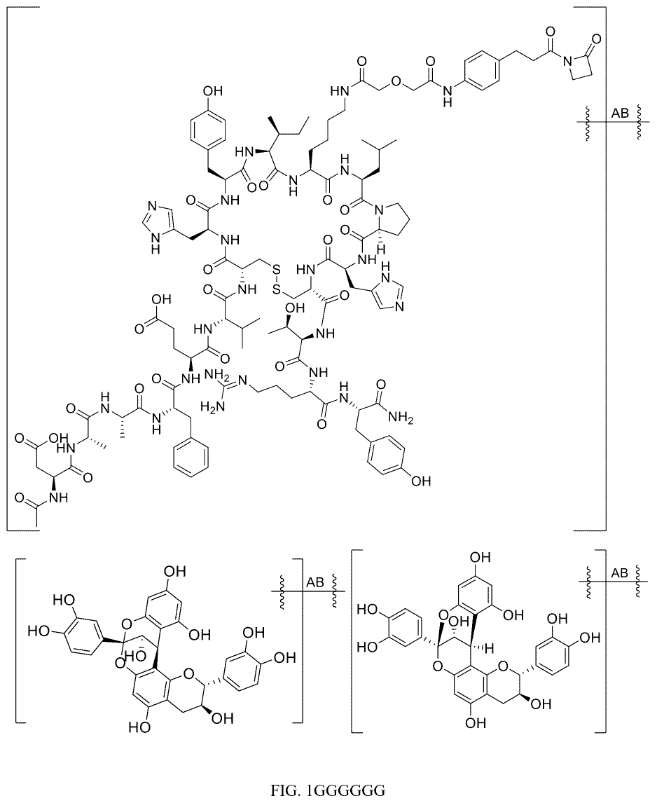

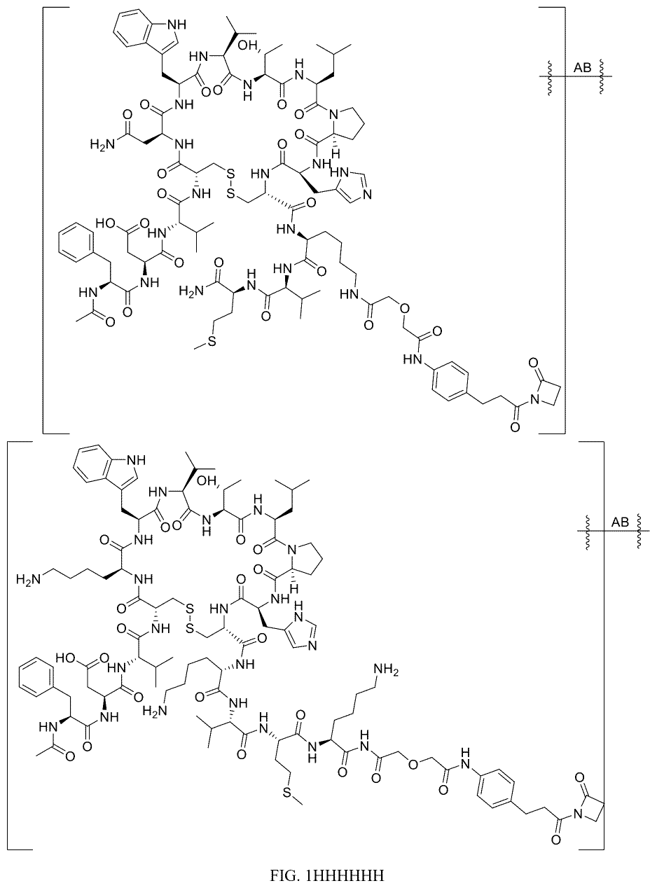

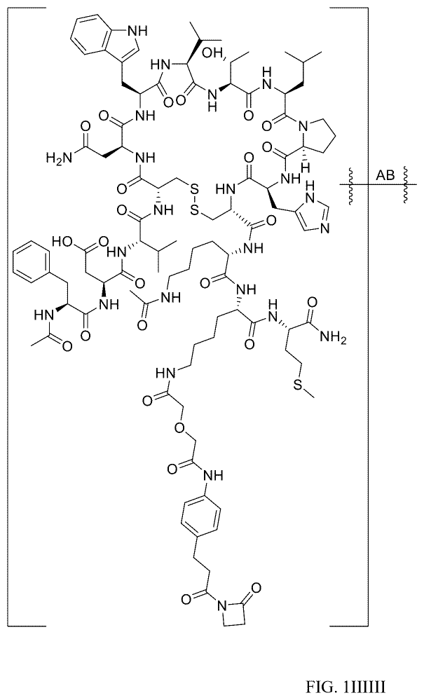

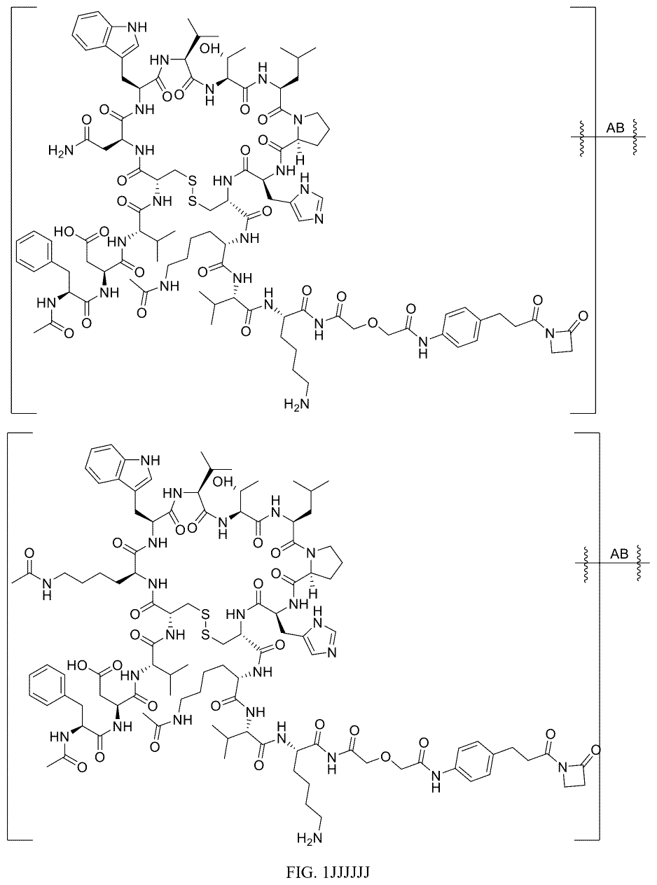

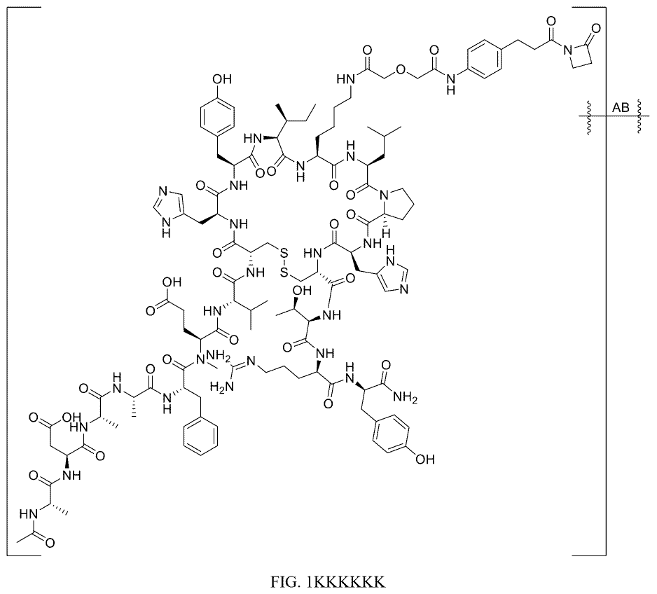

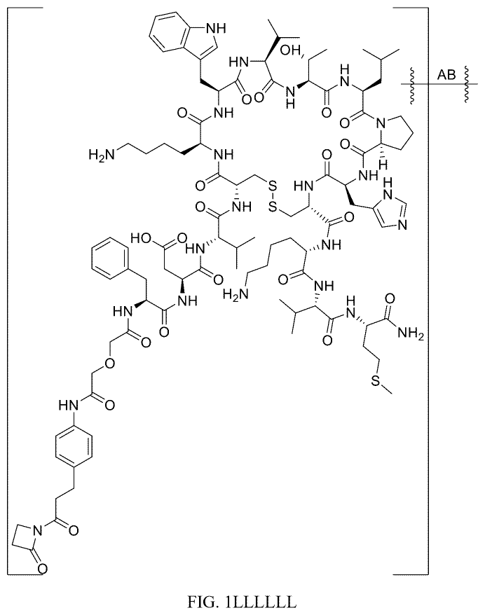

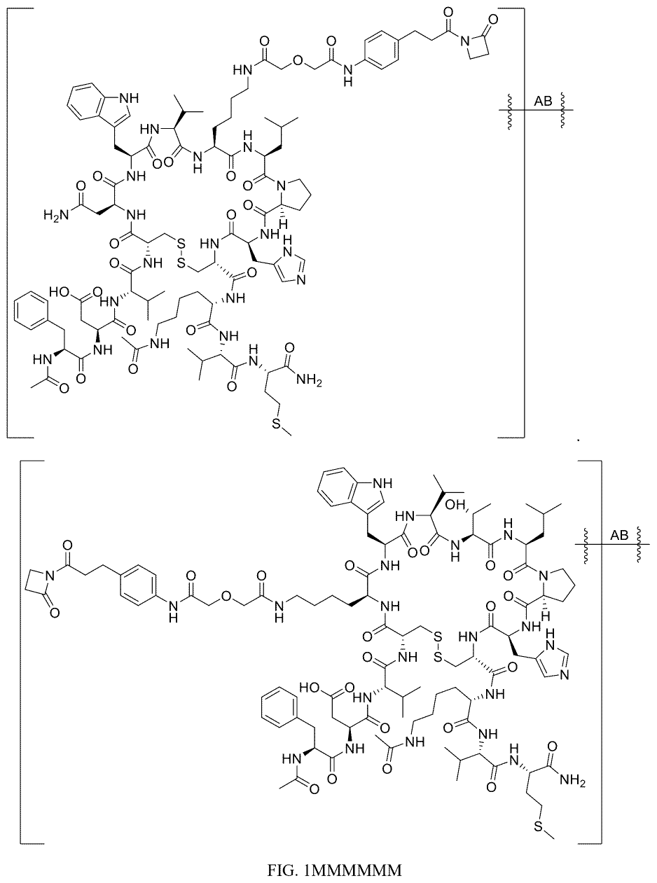

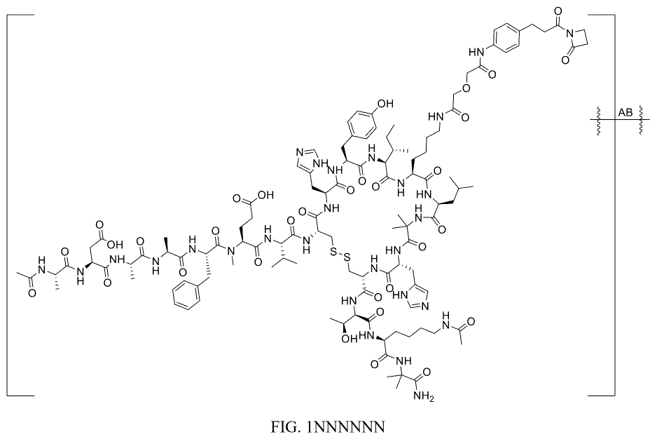

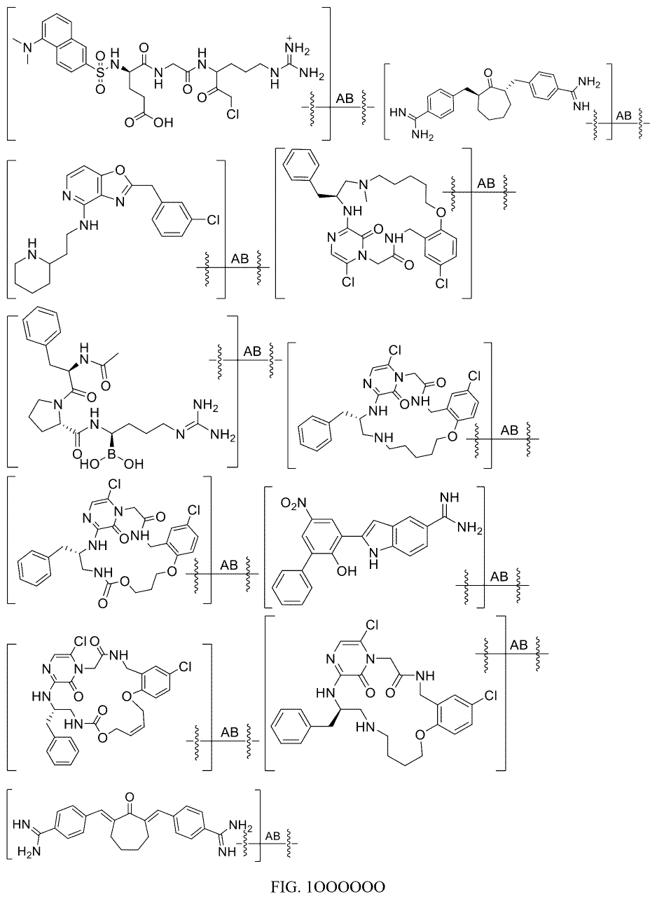

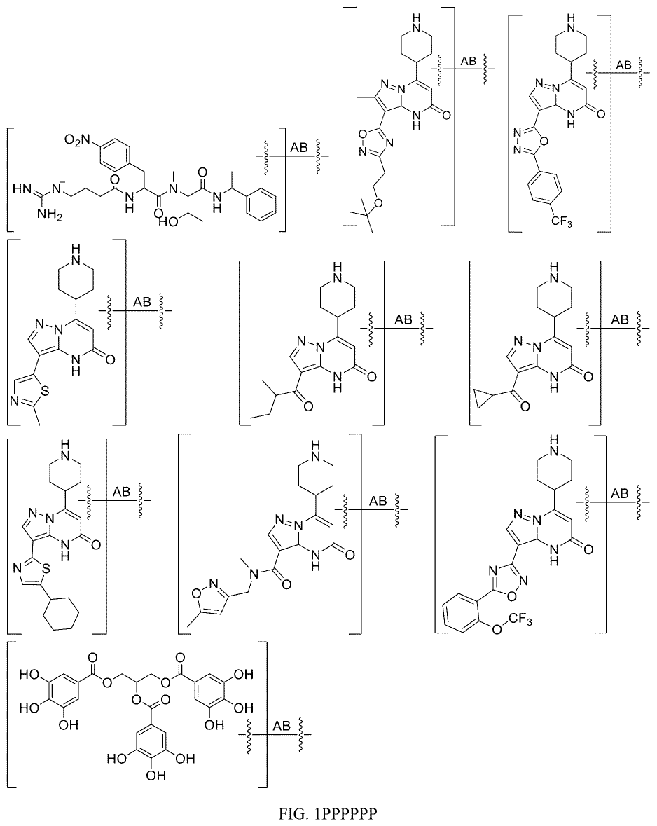

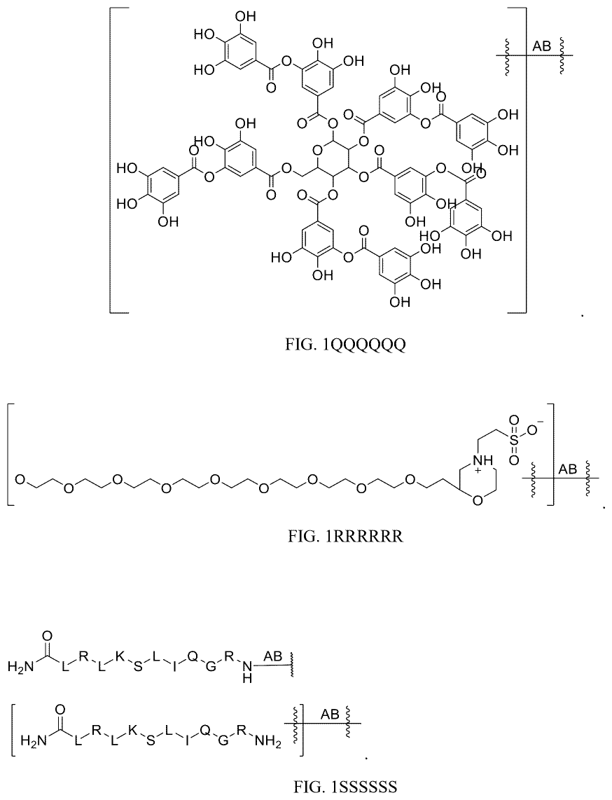

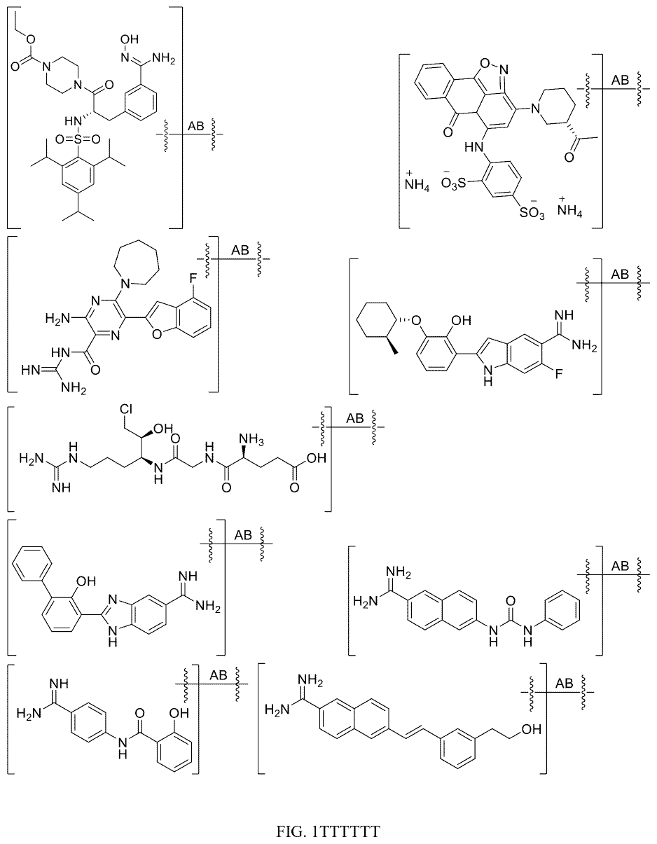

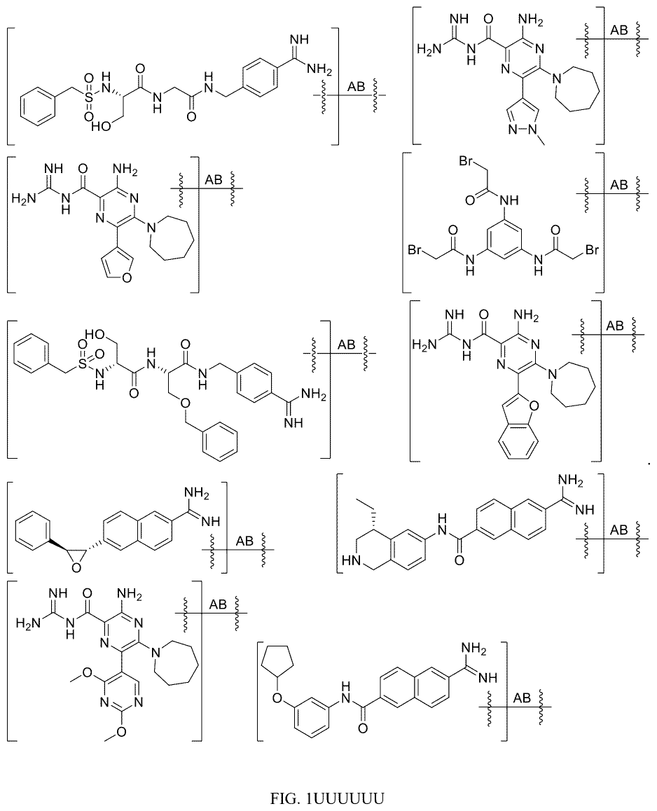

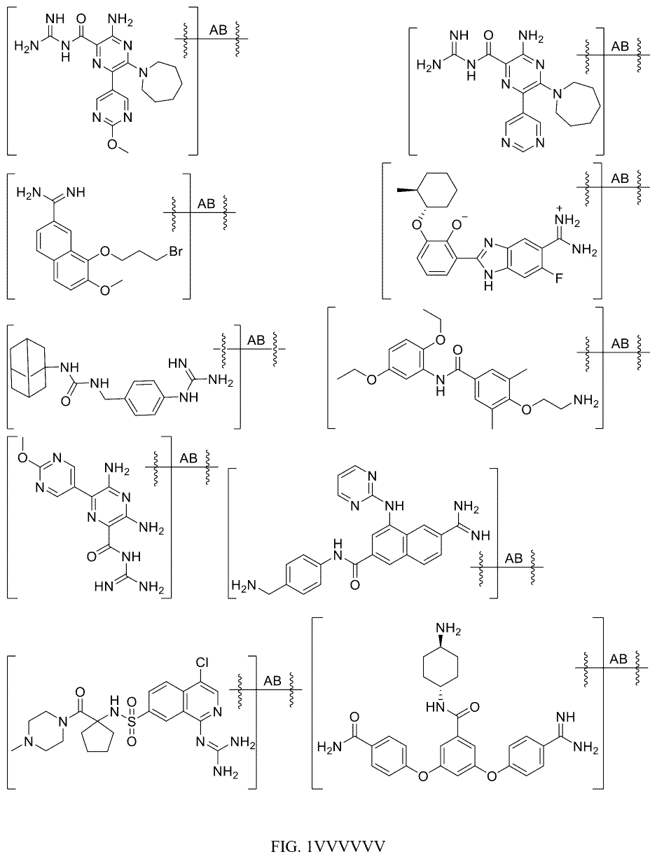

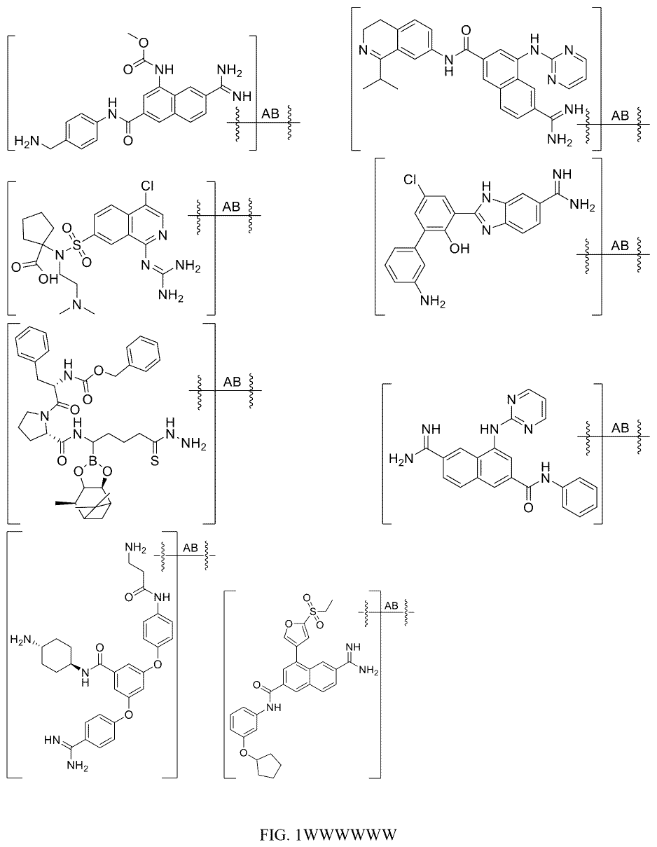

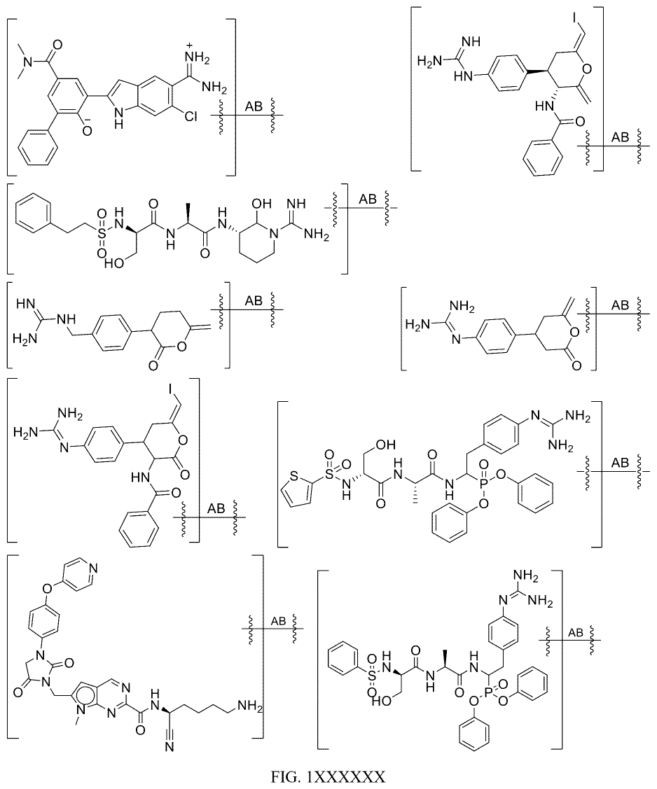

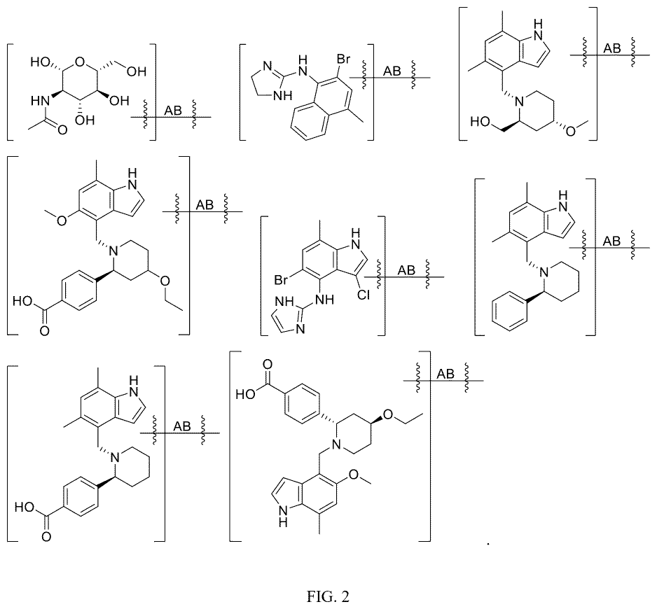

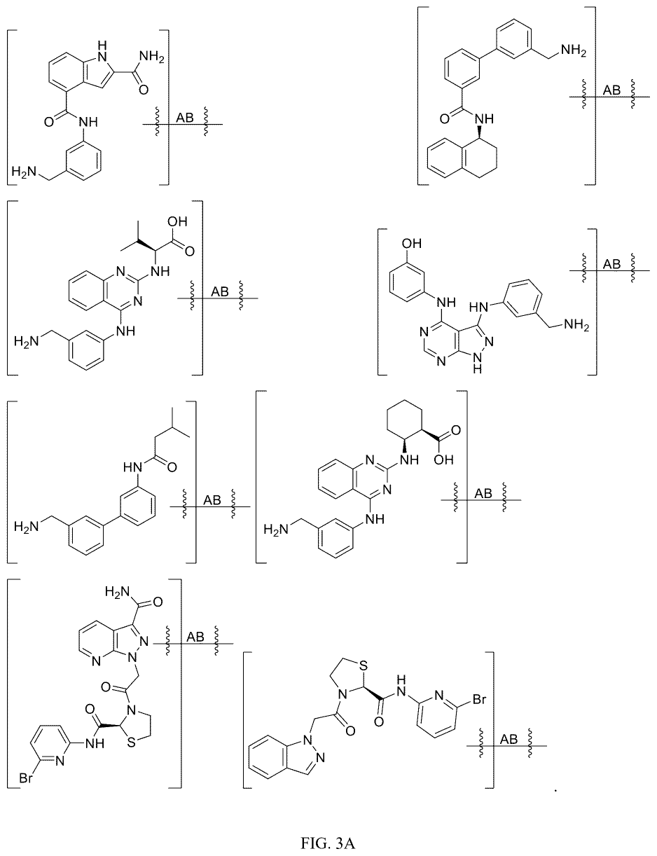

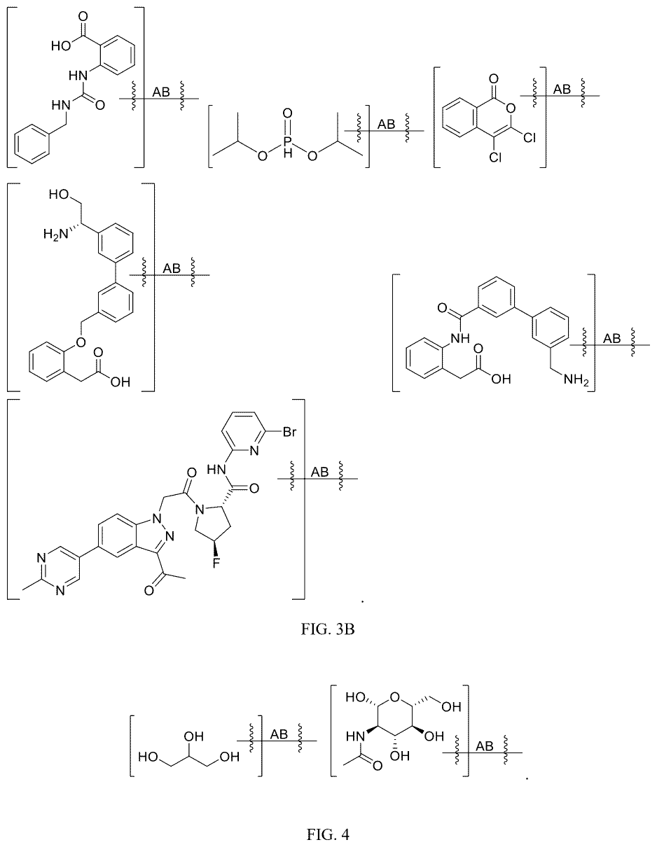

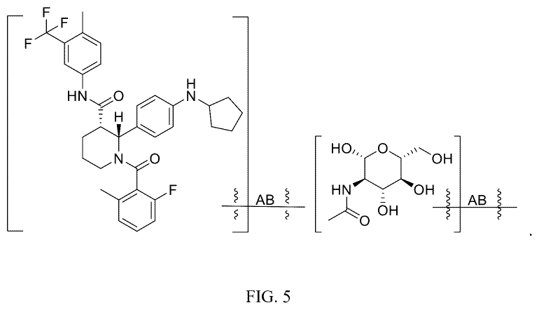

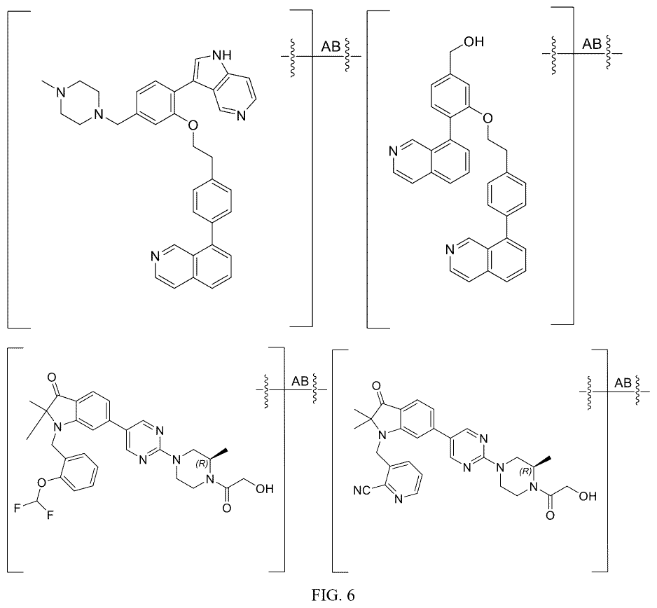

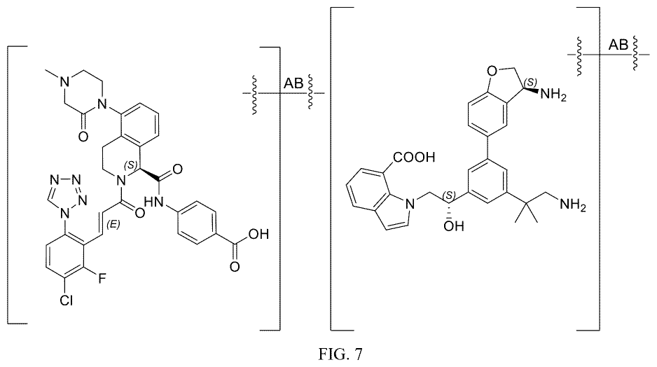

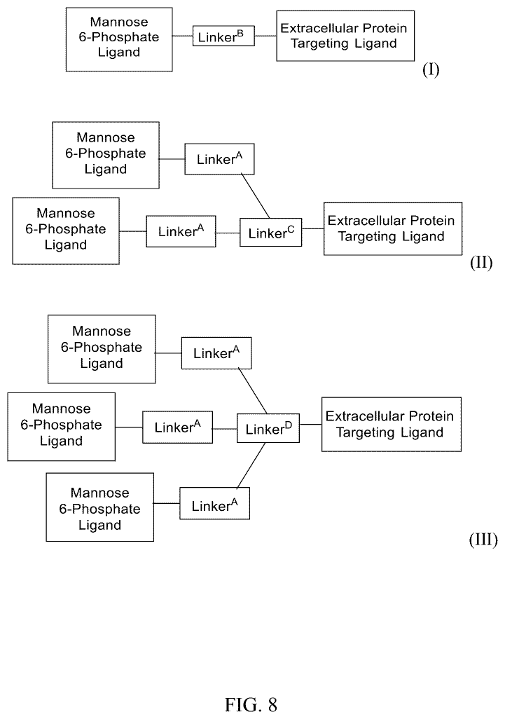

FIGS. 1T-1W provides a non-limiting list of Extracellular Protein Targeting Ligands that target Interleukin-6 (IL-6).

FIGS. 1X-1AA provides a non-limiting list of Extracellular Protein Targeting Ligands that target Interferon gamma (IFN-γ).

FIGS. 1BB-1KK provides a non-limiting list of Extracellular Protein Targeting Ligands that target Vascular endothelial growth factor (VEGF).

FIG. 1LL provides a non-limiting list of Extracellular Protein Targeting Ligands that target Transforming growth factor beta (TGF-β1).

FIGS. 1MM-1PP provides a non-limiting list of Extracellular Protein Targeting Ligands that target proprotein convertase subtilisin kexin 9 (PCSK-9).

FIGS. 1QQ-1SS provides a non-limiting list of Extracellular Protein Targeting Ligands that target Carboxypeptidase B2 (CPB2).

FIGS. 1TT-1UU provides a non-limiting list of Extracellular Protein Targeting Ligands that target Cholinesterase (ChE).

FIGS. 1VV-1WW provides a non-limiting list of Extracellular Protein Targeting Ligands that target C—C Motif Chemokine Ligand 2 (CCL2).

FIGS. 1XX-1BBB provides a non-limiting list of Extracellular Protein Targeting Ligands that target coagulation factor VII (Factor VII).

FIGS. 1CCC-1FFF provides a non-limiting list of Extracellular Protein Targeting Ligands that target coagulation factor IX (Factor IX).

FIG. 1GGG provides a non-limiting list of Extracellular Protein Targeting Ligands that target CD40 Ligand (CD40L).

FIGS. 1HHH-1JJJ provides a non-limiting list of Extracellular Protein Targeting Ligands that target coagulation factor Xa (Factor Xa).

FIGS. 1KKK-1MMM provides a non-limiting list of Extracellular Protein Targeting Ligands that target coagulation factor XI (Factor XI).

FIGS. 1NNN and 1OOO provides a non-limiting list of Extracellular Protein Targeting Ligands that target coagulation factor XII (Factor XII).

FIGS. 1PPP and 1QQQ provides a non-limiting list of Extracellular Protein Targeting Ligands that target coagulation factor XIII (Factor XIII).

FIGS. 1RRR-1UUU provides a non-limiting list of Extracellular Protein Targeting Ligands that target fibroblast growth factor 1 (FGF1).

FIGS. 1VVV-1XXX provides a non-limiting list of Extracellular Protein Targeting Ligands that target fibroblast growth factor 2 (FGF2).

FIGS. 1YYY and 1ZZZ provides a non-limiting list of Extracellular Protein Targeting Ligands that target fibronectin (FN1).

FIGS. 1AAAA and 1BBBB provides a non-limiting list of Extracellular Protein Targeting Ligands that target Interleukin-5 (IL-5).

FIG. 1CCCC provides a non-limiting list of Extracellular Protein Targeting Ligands that target Interleukin-8 (IL-8).

FIGS. 1DDDD and 1EEEE provides a non-limiting list of Extracellular Protein Targeting Ligands that target Interleukin-10 (IL-10).

FIGS. 1FFFF and 1GGGG provides a non-limiting list of Extracellular Protein Targeting Ligands that target Interleukin-21 (IL-21).

FIGS. 1HHHH and 1IIII provides a non-limiting list of Extracellular Protein Targeting Ligands that target Interleukin-22 (IL-22).

FIGS. 1JJJJ-INNNN provides a non-limiting list of Extracellular Protein Targeting Ligands that target Kallikrein 1.

FIG. 1OOOO provides a non-limiting list of Extracellular Protein Targeting Ligands that target lipoprotein lipase (LPL).

FIGS. 1PPPP and 1QQQQ provides a non-limiting list of Extracellular Protein Targeting Ligands that target matrix metalloproteinase-1 (MMP1).

FIGS. 1RRRR-1DDDDD provides a non-limiting list of Extracellular Protein Targeting Ligands that target Macrophage migration inhibitory factor (MIF), also known as glycosylation-inhibiting factor (GIF), L-dopachrome isomerase, or phenylpyruvate tautomerase.

FIGS. 1EEEEE-1GGGGG provides a non-limiting list of Extracellular Protein Targeting Ligands that target neutrophil elastase (NE).

FIGS. 1HHHHH and 1IIIII provides a non-limiting list of Extracellular Protein Targeting Ligands that target Prothrombin.

FIGS. 1JJJJJ-INNNNN provides a non-limiting list of Extracellular Protein Targeting Ligands that target Plasma kallikrein (KLKB1).

FIGS. 1OOOOO-1SSSSS provides a non-limiting list of Extracellular Protein Targeting Ligands that target plasminogen (PLG).

FIGS. 1TTTTT-1XXXXX provides a non-limiting list of Extracellular Protein Targeting Ligands that target Plasminogen activator inhibitor-1 (PAI-1), endothelial plasminogen activator inhibitor or serpin E1.

FIGS. 1YYYYY-1AAAAAA provides a non-limiting list of Extracellular Protein Targeting Ligands that target phospholipases A2, for example type 1B or group 1B (PLA2, PA21B, PLA2G1B, PLA2-IB).

FIGS. 1BBBBBB-1DDDDDD provides a non-limiting list of Extracellular Protein Targeting Ligands that target phospholipases A2, for example type IIA or group IIA (PLA2, PLA2A, PA2IIA, PLA2G2A, PLA2-IIA).

FIGS. 1EEEEEE-1NNNNNN provides a non-limiting list of Extracellular Protein Targeting Ligands that target placental growth factor (PGF).

FIGS. 1OOOOOO-1QQQQQQ provides a non-limiting list of Extracellular Protein Targeting Ligands that target plasminogen activator, tissue type (tPA, PLAT).

FIG. 1RRRRRR provides a non-limiting list of Extracellular Protein Targeting Ligands that target Transforming growth factor beta 2 (TGF-B2, TGFB2).

FIG. 1SSSSSS provides a non-limiting list of Extracellular Protein Targeting Ligands that target thrombospondin 1 (TSP1, TSP-1, THBS1).

FIGS. 1TTTTTT-1 XXXXXX provides a non-limiting list of Extracellular Protein Targeting Ligands that target Urokinase or Urokinase-type plasminogen activator (UPA, uPA).

FIG. 2 provides a non-limiting list of exemplary Extracellular Protein Targeting Ligands that target complement factor B.

FIGS. 3A and 3B provides a non-limiting list of exemplary Extracellular Protein Targeting Ligands that target complement factor D.

FIG. 4 provides a non-limiting list of exemplary Extracellular Protein Targeting Ligands that target complement factor H.

FIG. 5 provides a non-limiting list of exemplary Extracellular Protein Targeting Ligands that target complement component 5.

FIG. 6 provides a non-limiting list of exemplary Extracellular Protein Targeting Ligands that target TNF-alpha.

FIG. 7 provides a non-limiting list of exemplary Extracellular Protein Targeting Ligands that target factor XI.

FIG. 8 provides a non-limiting list of exemplary formulas of the present invention.

FIG. 9 provides a pharmacokinetic plot of Compound 12. The y-axis is compound concentration in plasma and the x-axis is time. The experimental procedure is described in Example 3.

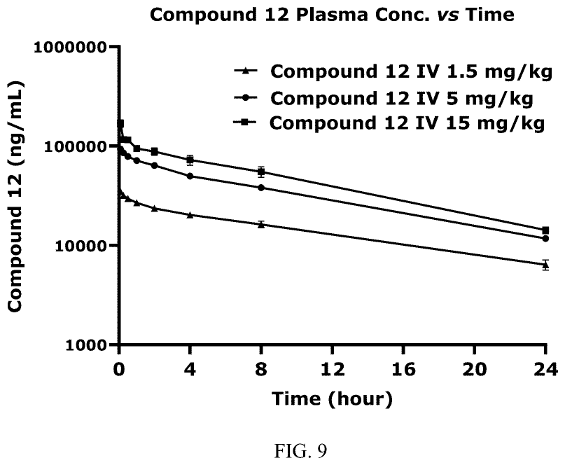

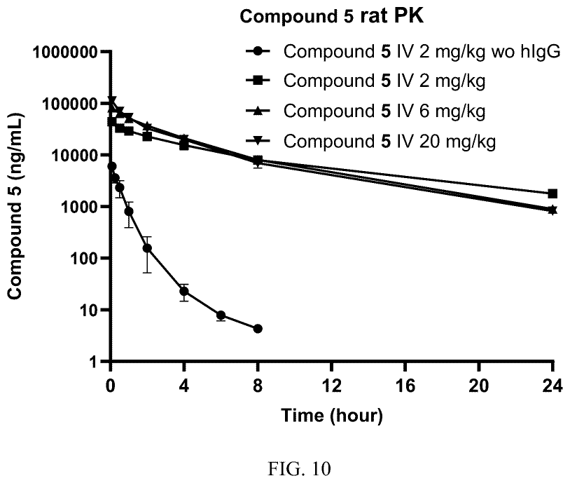

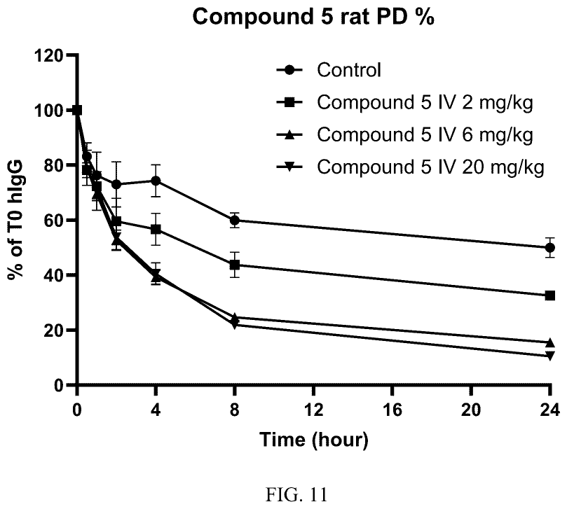

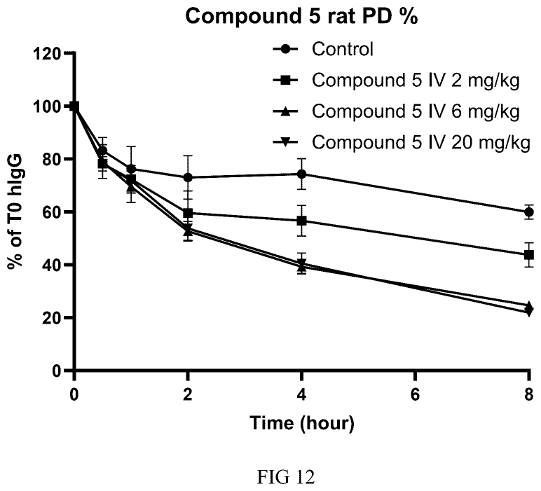

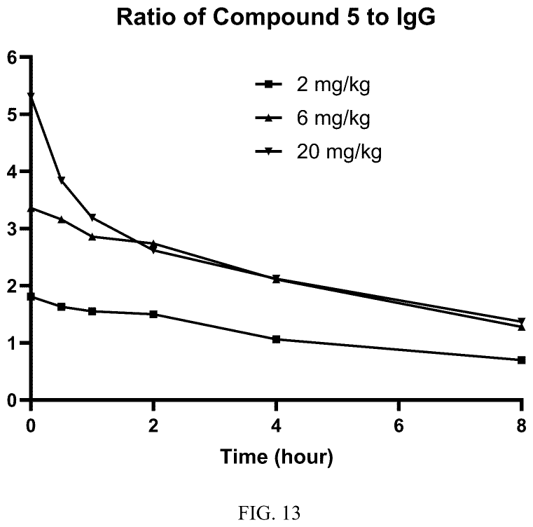

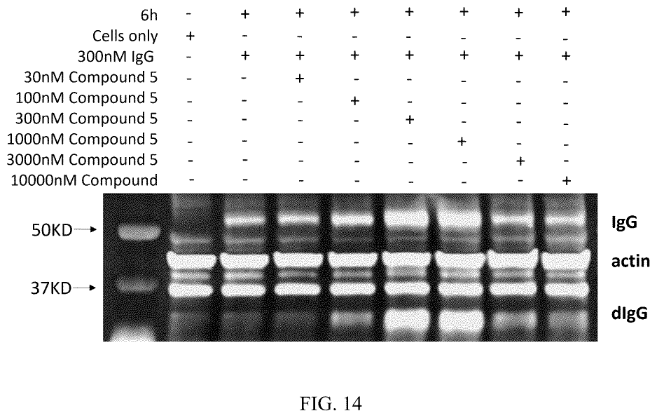

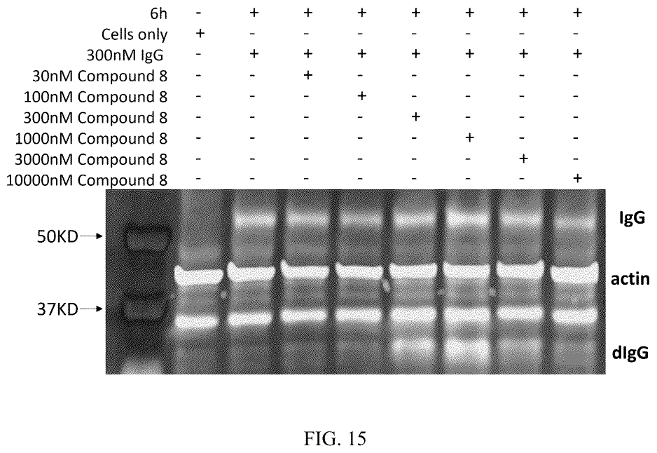

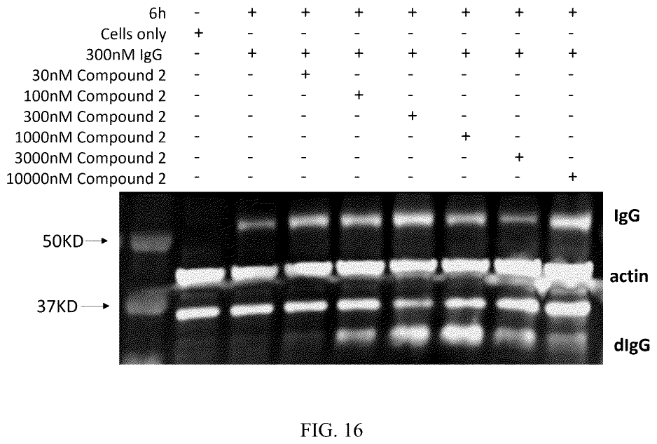

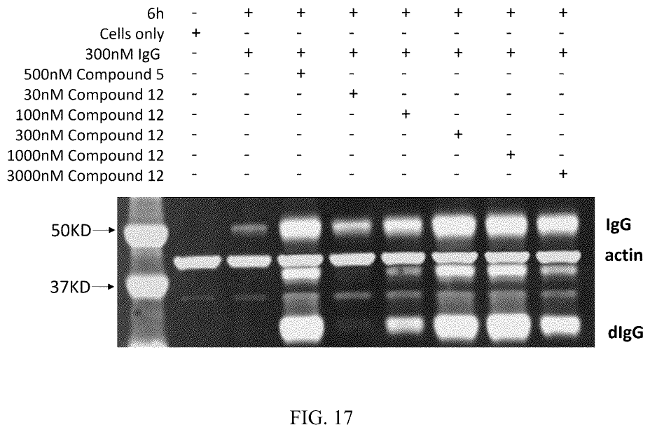

FIG. 10 provides a pharmacokinetic plot of Compound 5. The y-axis is compound concentration in plasma and the x-axis is time. The experimental procedure is described in Example 3.

FIG. 11 provides a plot of the degradation of coadministered human IgG (hlgG) by Compound 5 in rats. The y-axis is the percent of initial hlgG and the x axis is time. The experimental procedure is described in Example 3.

FIG. 12 provides a plot of the degradation of coadministered human IgG (hlgG) by Compound 5 in rats. The y-axis is the percent of initial hlgG and the x axis is time. The experimental procedure is described in Example 3.

FIG. 13 is a plot of the ratio of Compound 5 to IgG over time. The y-axis is the ratio of Compound 5 to IgG and the x-axis is time. The experimental procedure is described in Example 3.

FIG. 14 is a gel electrophoresis image showing degradation of IgG by Compound 5. The experimental procedure is described in Example 5.

FIG. 15 is a gel electrophoresis image showing degradation of IgG by Compound 8. The experimental procedure is described in Example 5.

FIG. 16 is a gel electrophoresis image showing degradation of IgG by Compound 2. The experimental procedure is described in Example 5.

FIG. 17 is a gel electrophoresis image showing degradation of IgG by Compound 12. The experimental procedure is described in Example 5.

### DETAILED DESCRIPTION OF THE INVENTION

Novel compounds and their pharmaceutically acceptable salts and compositions thereof that degrade disease-mediating extracellular proteins, as well as starting materials and intermediates for such compounds and their methods of use and processes of manufacture are provided. This invention provides novel modifications of mannose 6-phosphate for use in extracellular protein degradation. These compounds can be used for intracorporeal therapeutic bloodpheresis.

### I. Mannose-Based Extracellular Protein Degraders of the Present Invention

In certain embodiments the extracellular protein degrading compound is selected from:

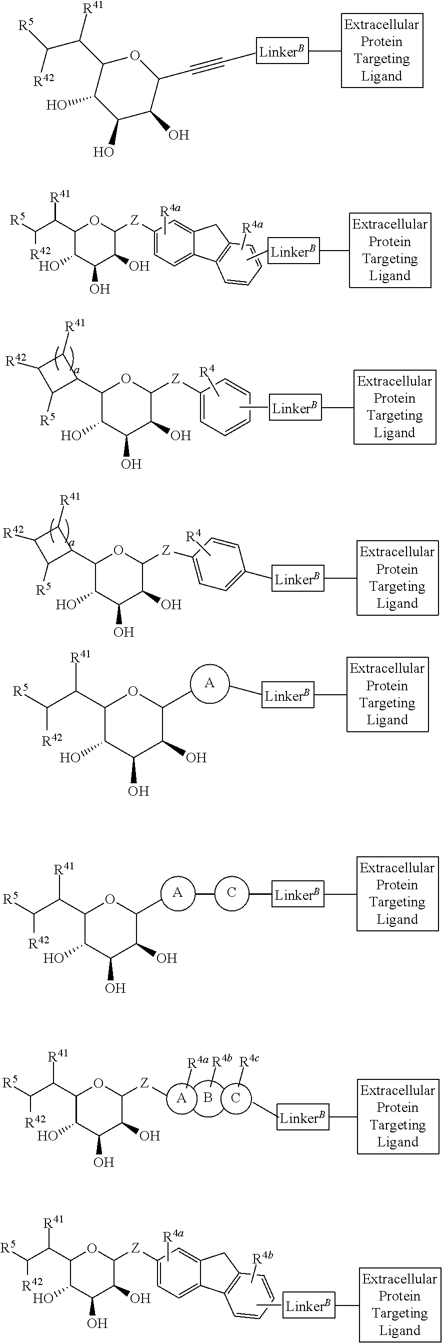

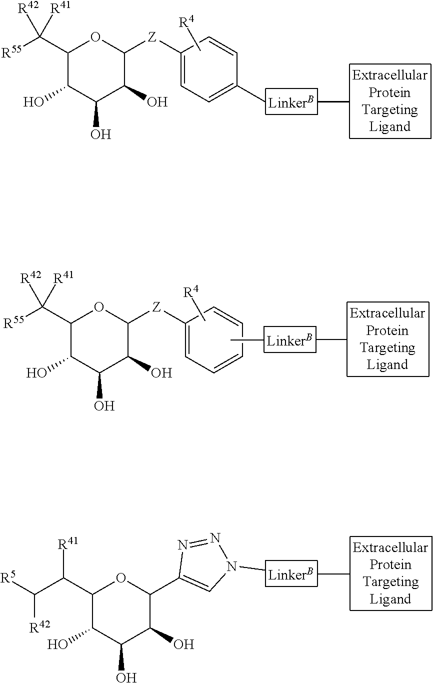

or a pharmaceutically acceptable salt thereof.

In other embodiments the extracellular protein degrading compound is a bi-(Formula II) or tri-(Formula III) version of the above structures. Non-limiting examples of compounds of Formula II include:

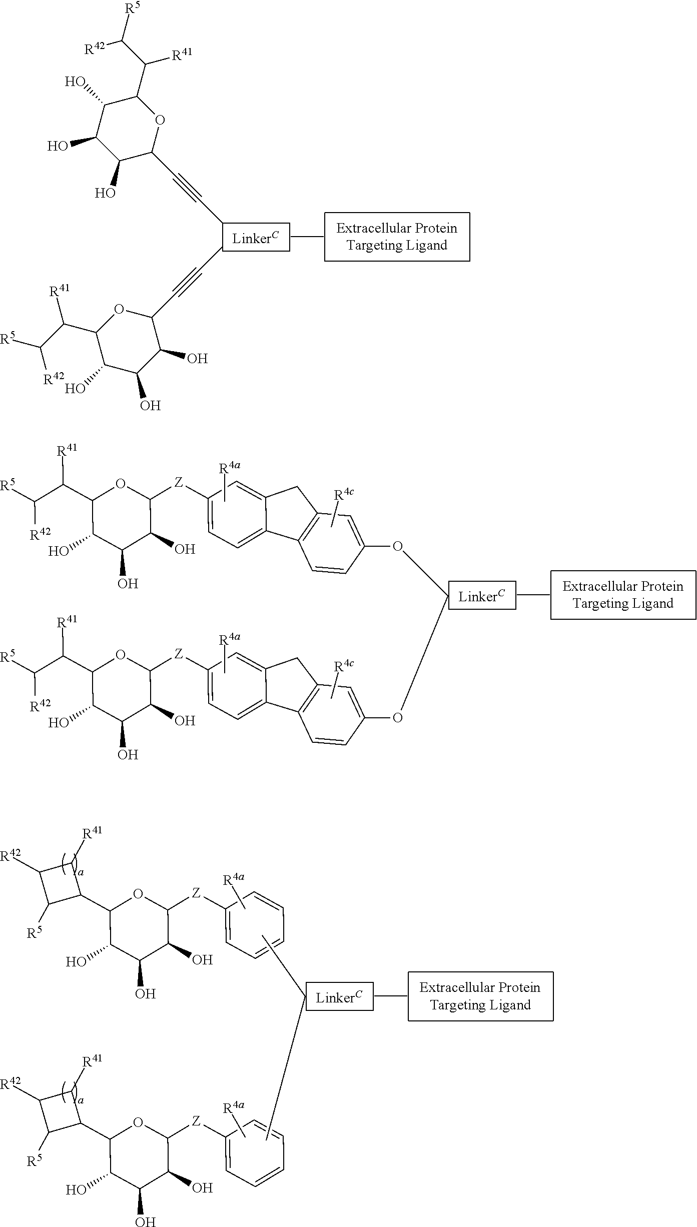

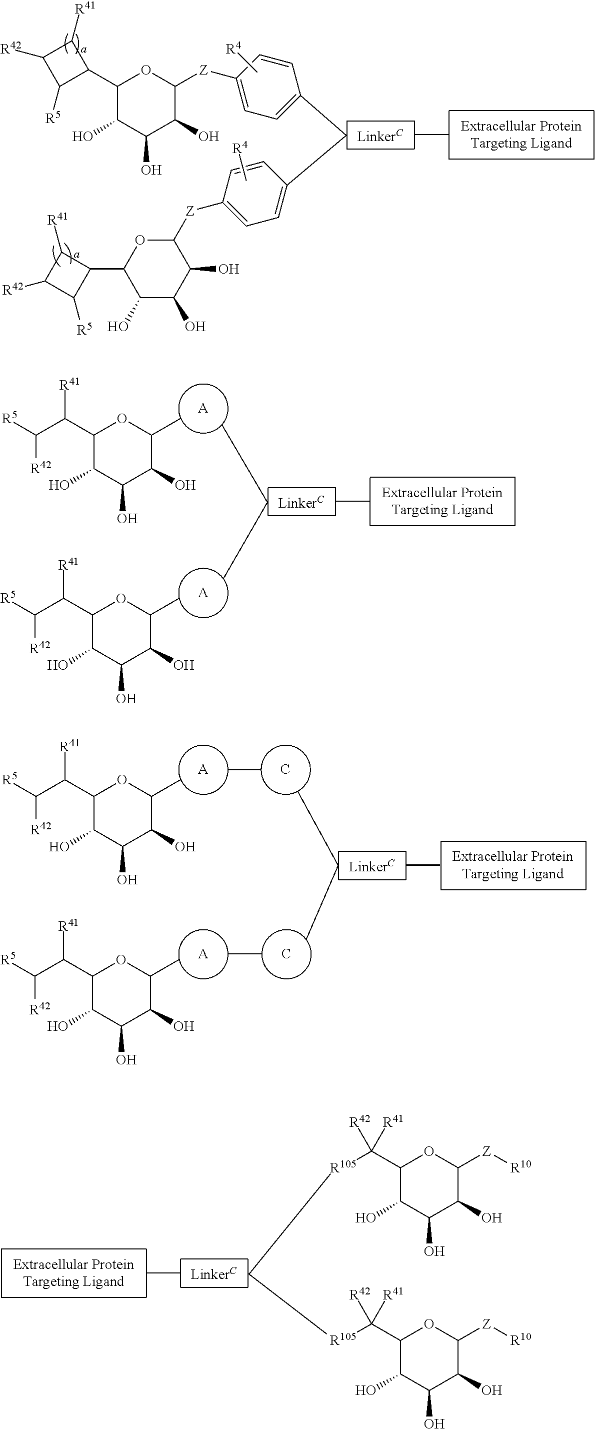

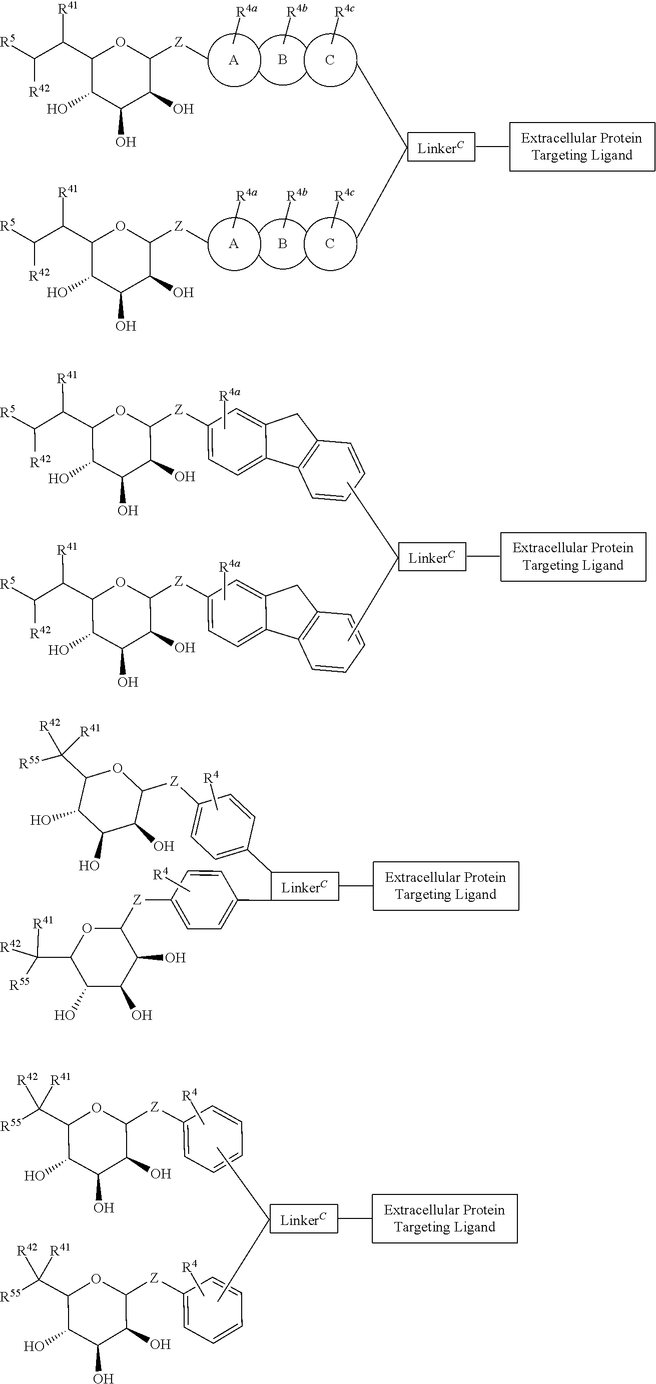

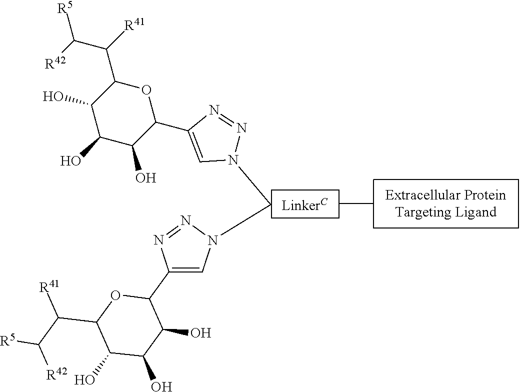

or a pharmaceutically acceptable salt thereof.

In certain embodiments, the extracellular protein degrading compound is of Formula III. Non-limiting examples of compounds of Formula III include:

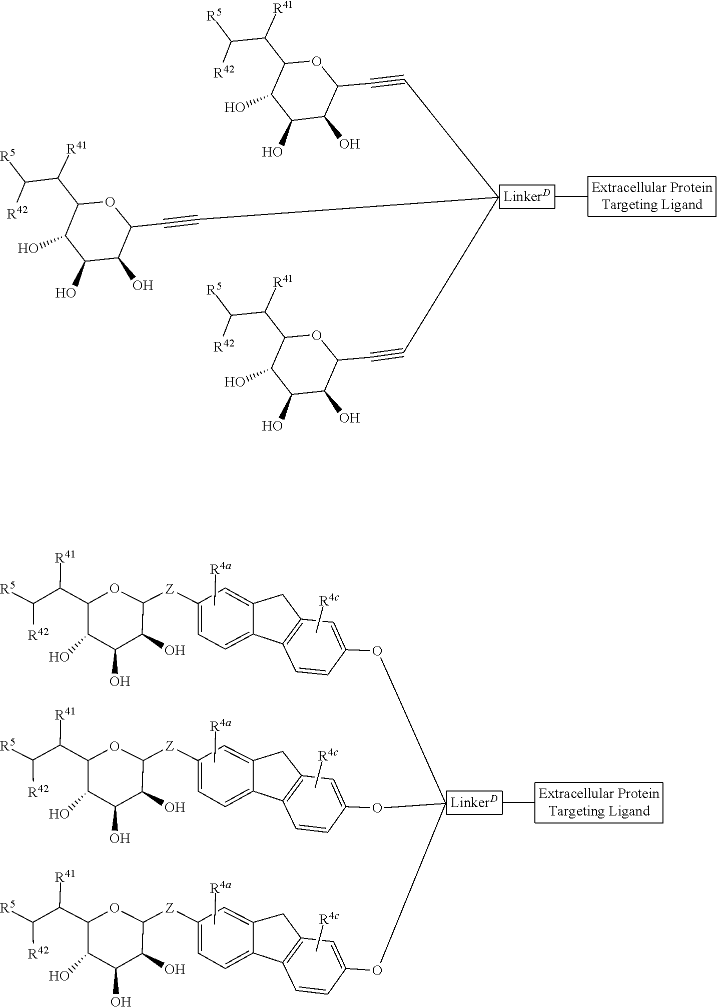

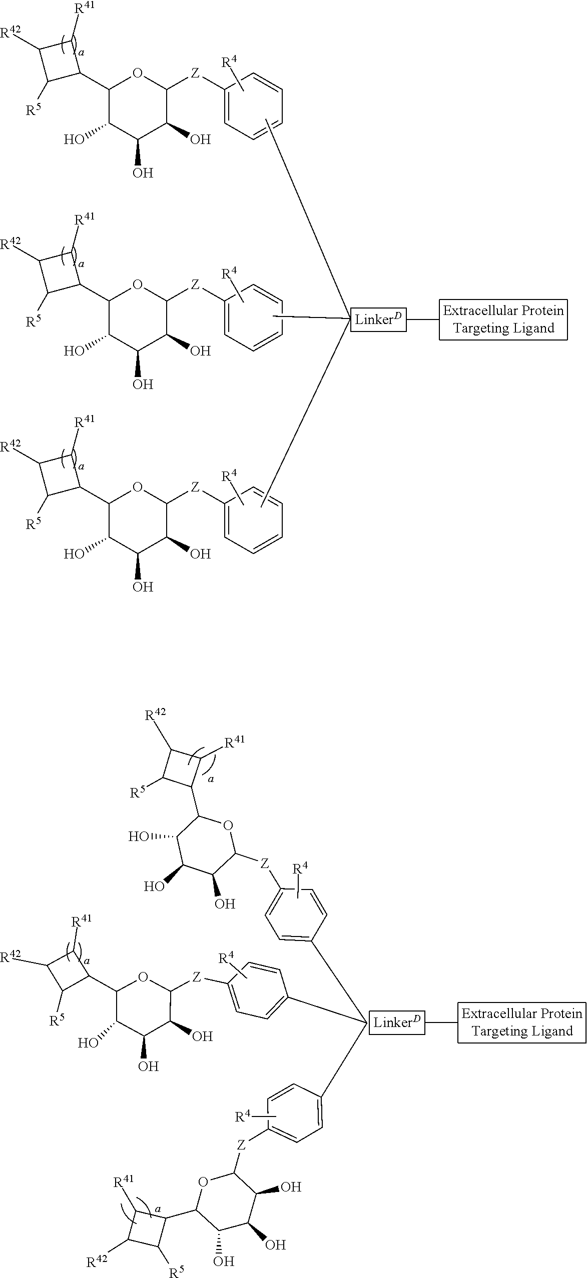

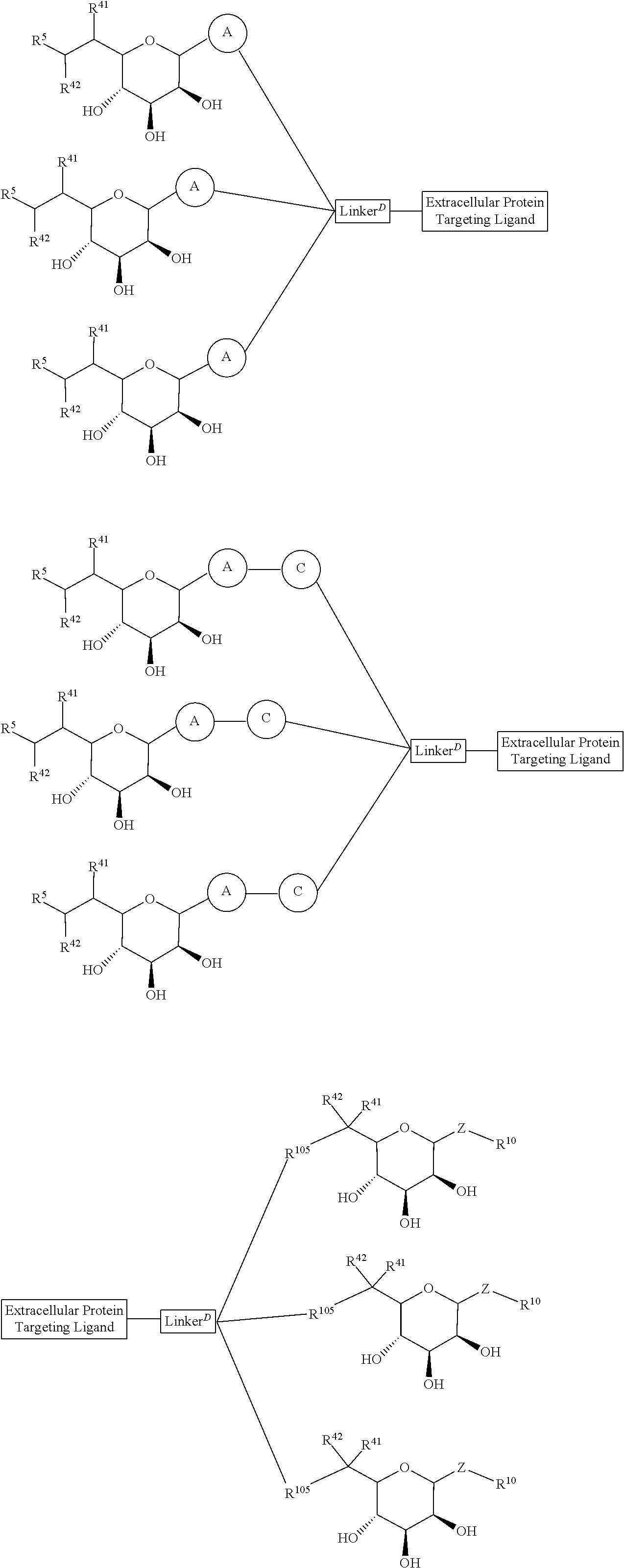

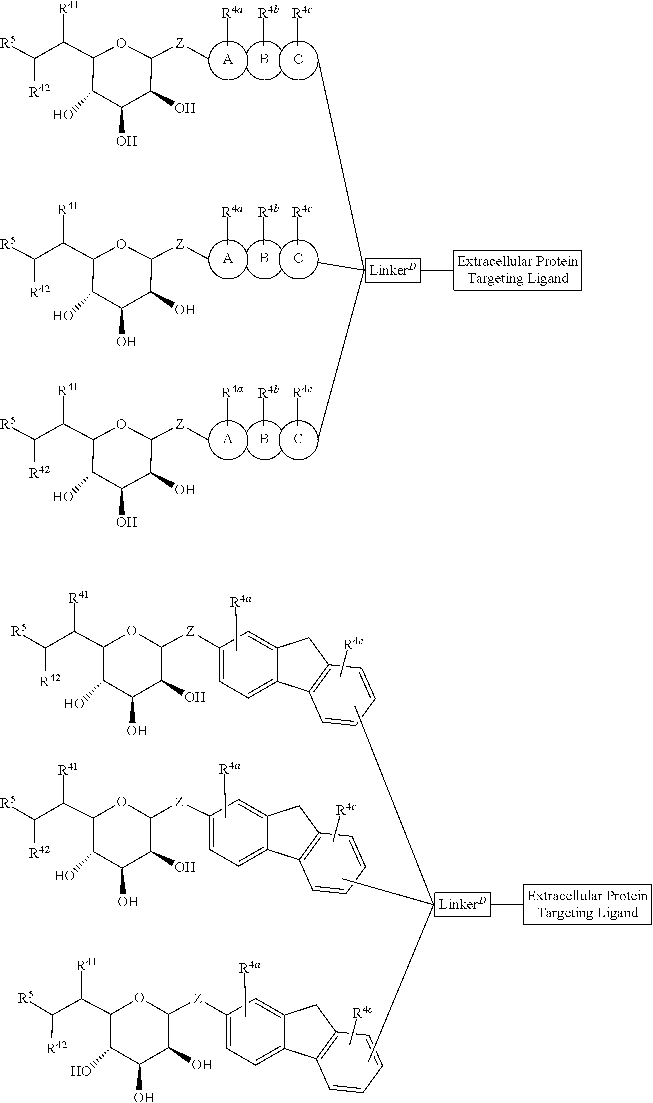

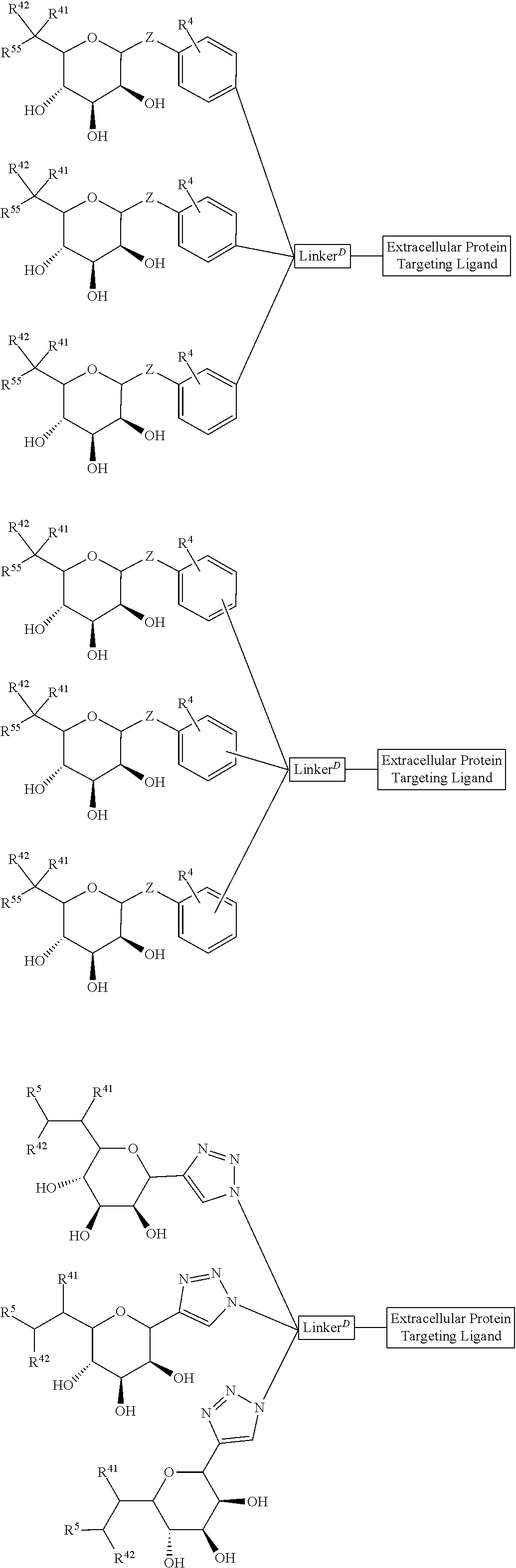

or a pharmaceutically acceptable salt thereof.

As used in the embodiments here, xx is independently selected from 0, 1, 2, 3, 4, 5, 6, 7, 8, 9, 10, 11, 12, 13, 14, 15, 16, 17, 18, 19, 20, 21, 22, 23, 24, and 25.
or each xx is independently selected from 0, 1, 2, 3, 4, 5, 6, 7, 8, 9, 10, 11, 12, 13, 14, 15, 16, 17, 18, 19, and 20.

As used in the embodiments here, yy is independently selected from 0, 1, 2, 3, 4, 5, 6, 7, 8, 9, 10, 11, 12, 13, 14, 15, 16, 17, 18, 19, 20, 21, 22, 23, 24, and 25.
or each yy is independently selected from 0, 1, 2, 3, 4, 5, 6, 7, 8, 9, 10, 11, 12, 13, 14, 15, 16, 17, 18, 19, and 20.

In alternative embodiments the compound of the present invention is selected from:

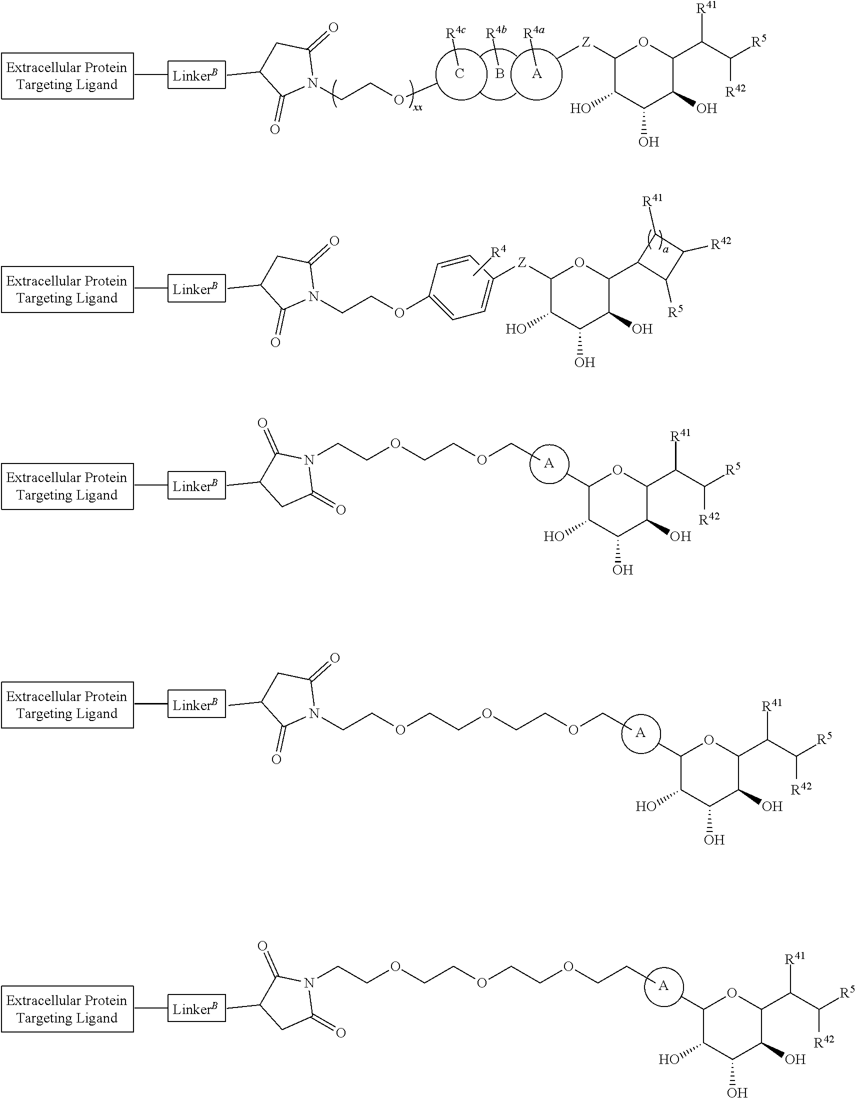

In alternative embodiments the compound of the present invention is selected from:

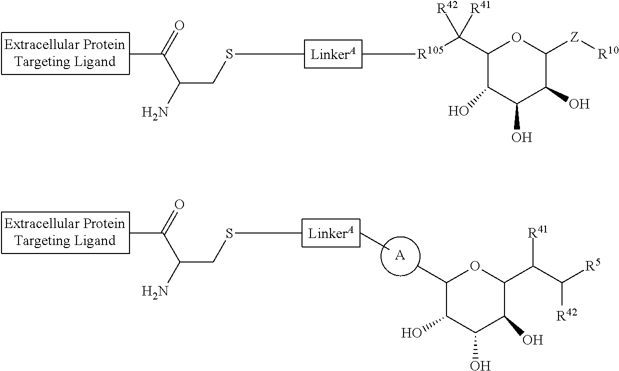

In alternative embodiments the compound of the present invention is selected from:

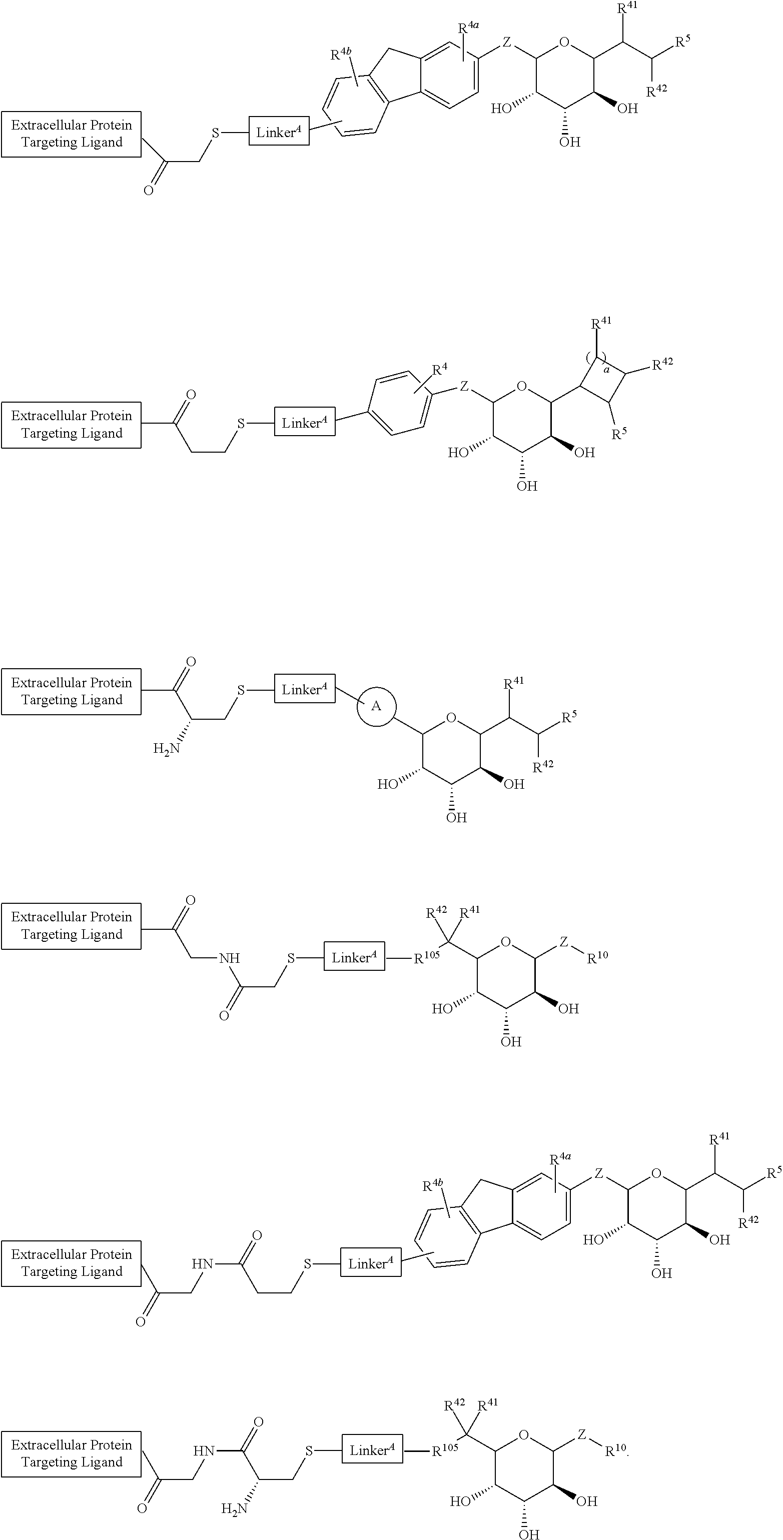

In alternative embodiments the compound of the present invention is selected from:

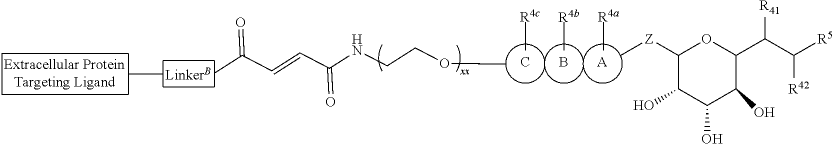

In alternative embodiments the compound of the present invention is selected from:

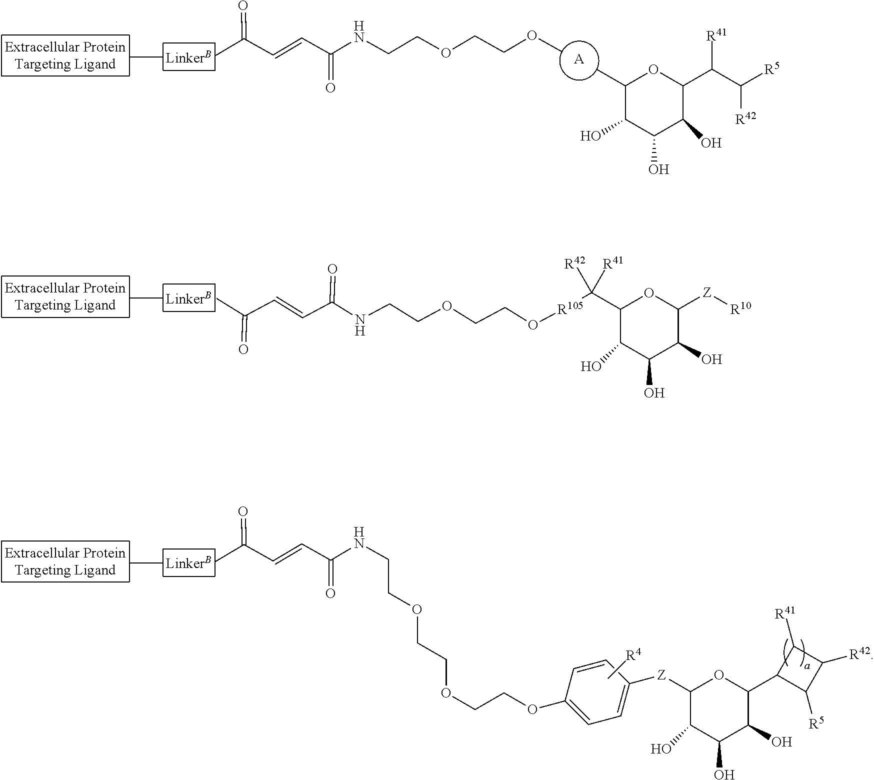

In certain embodiments the compound of the present invention is selected from:

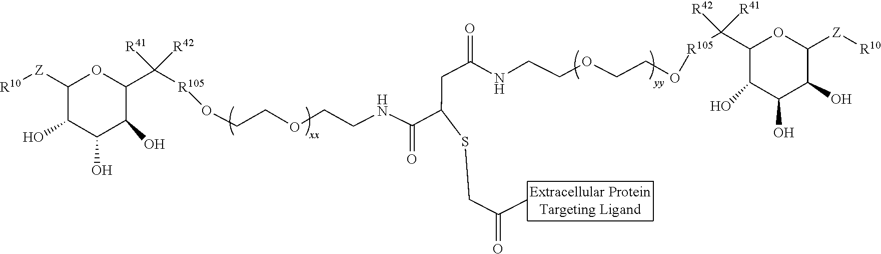

In certain embodiments, the Mannose 6-Phosphate Ligand is selected from:

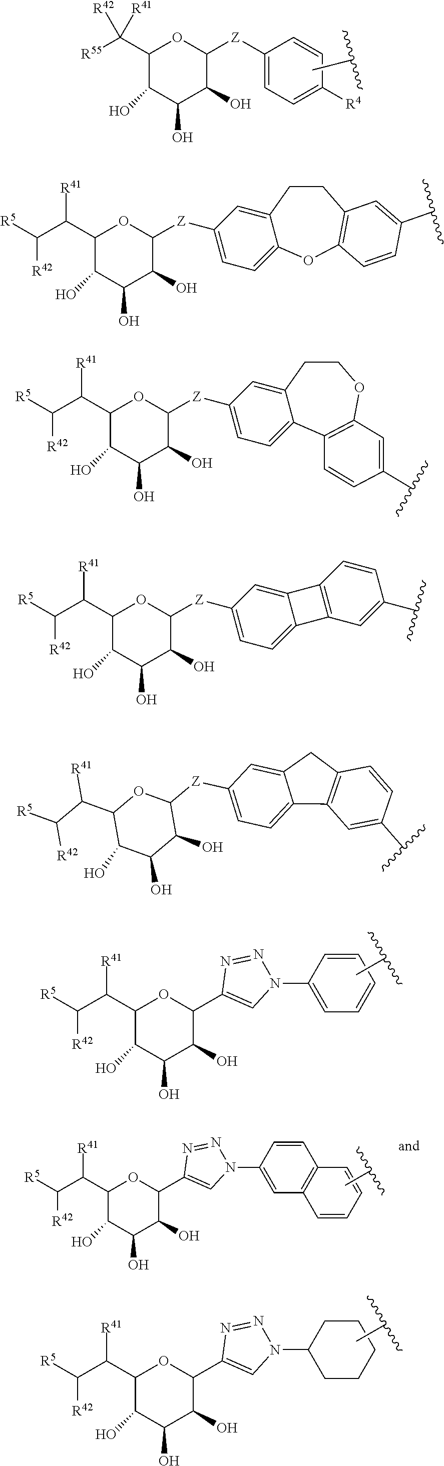

In certain embodiments, the Mannose 6-Phosphate Ligand is selected from:

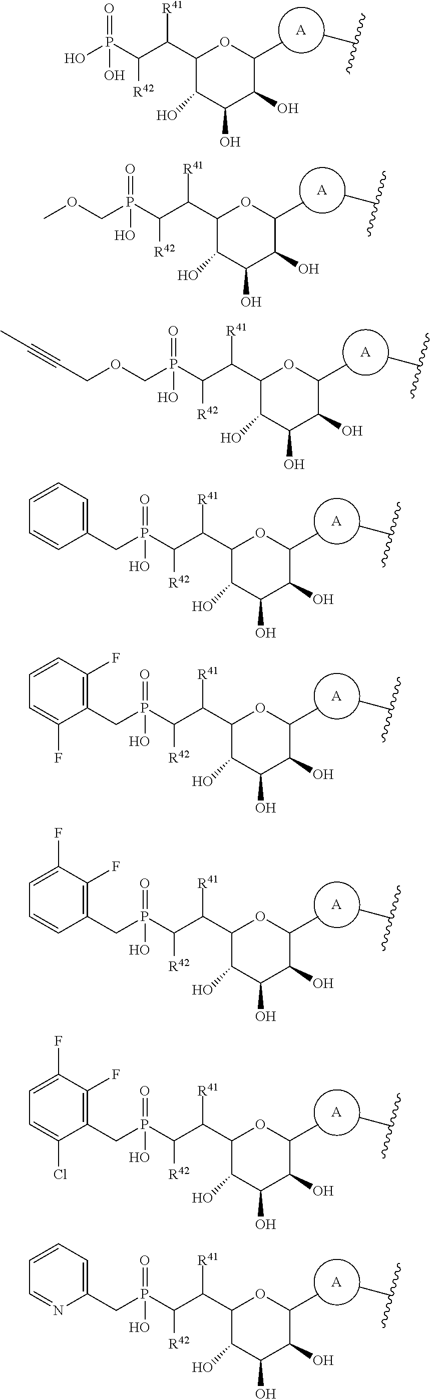

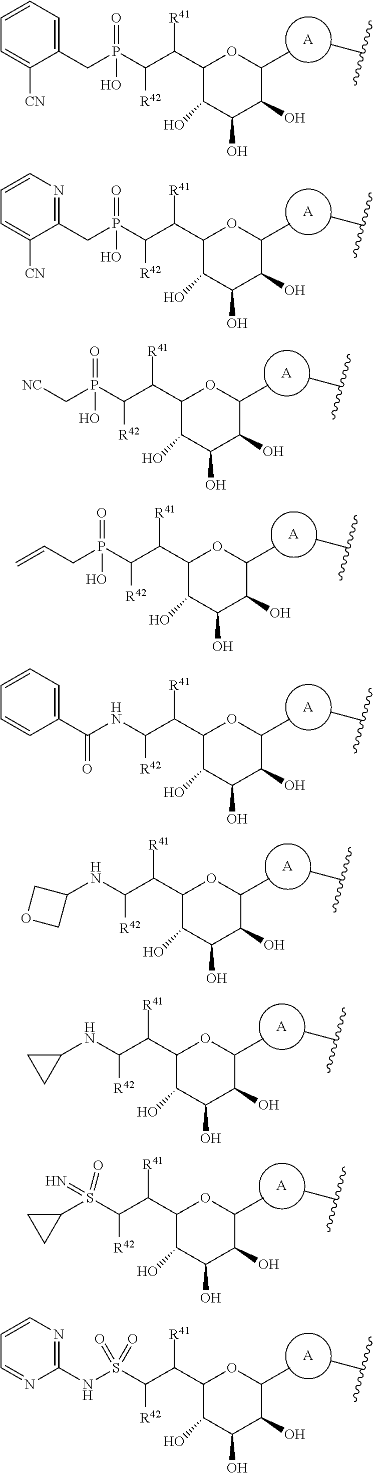

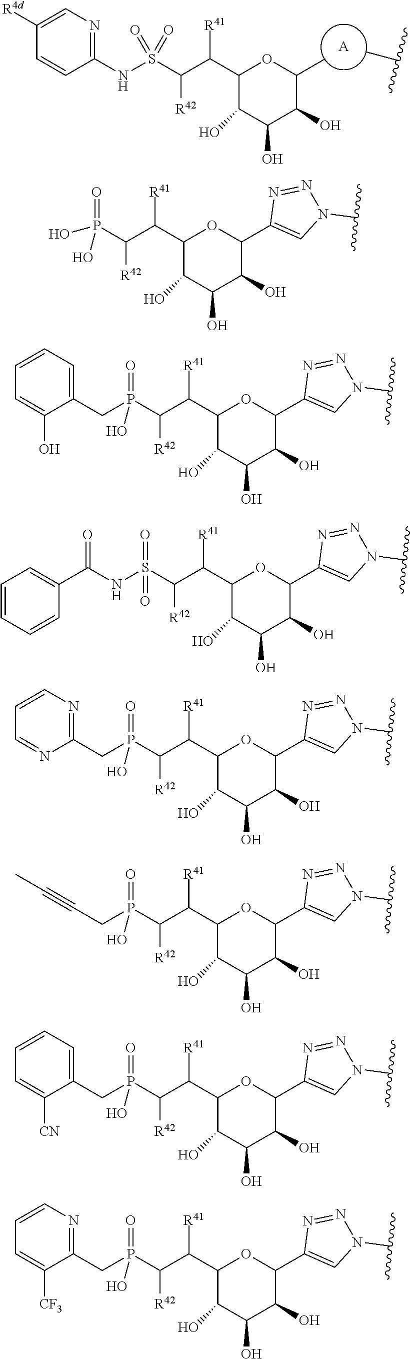

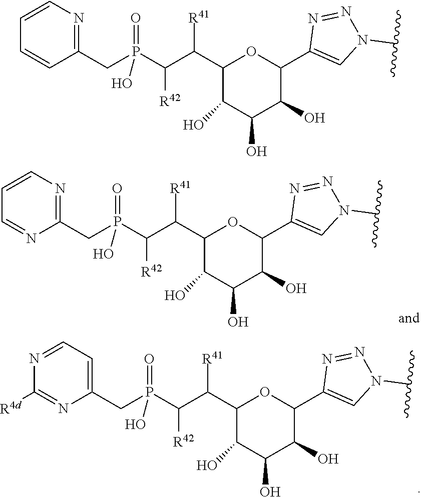

In certain embodiments, the Mannose 6-Phosphate Ligand is selected from:

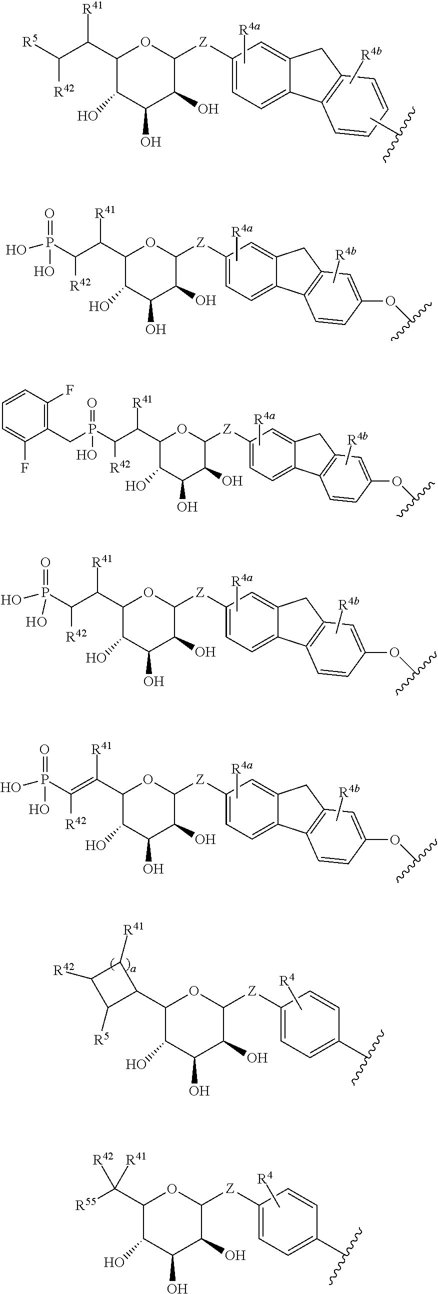

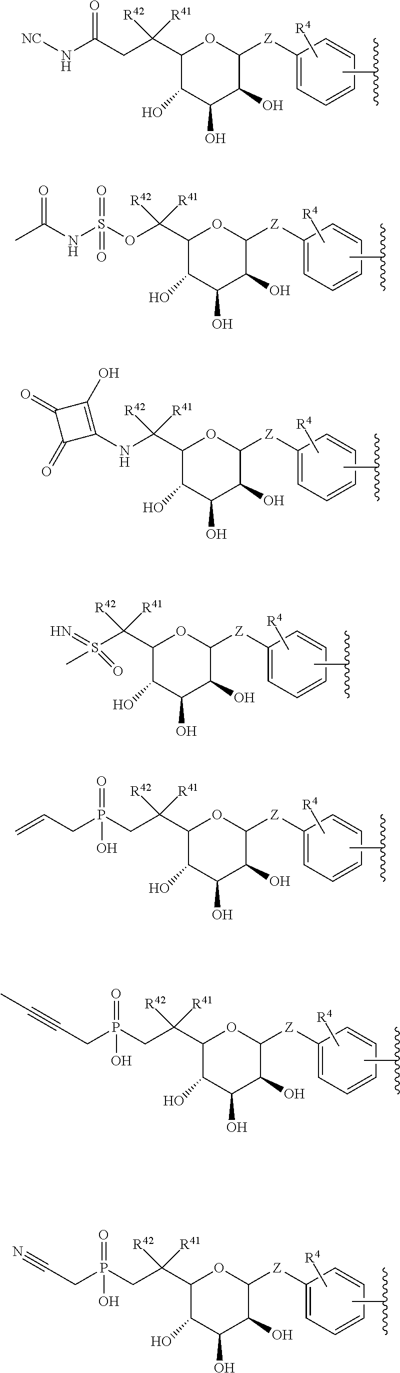

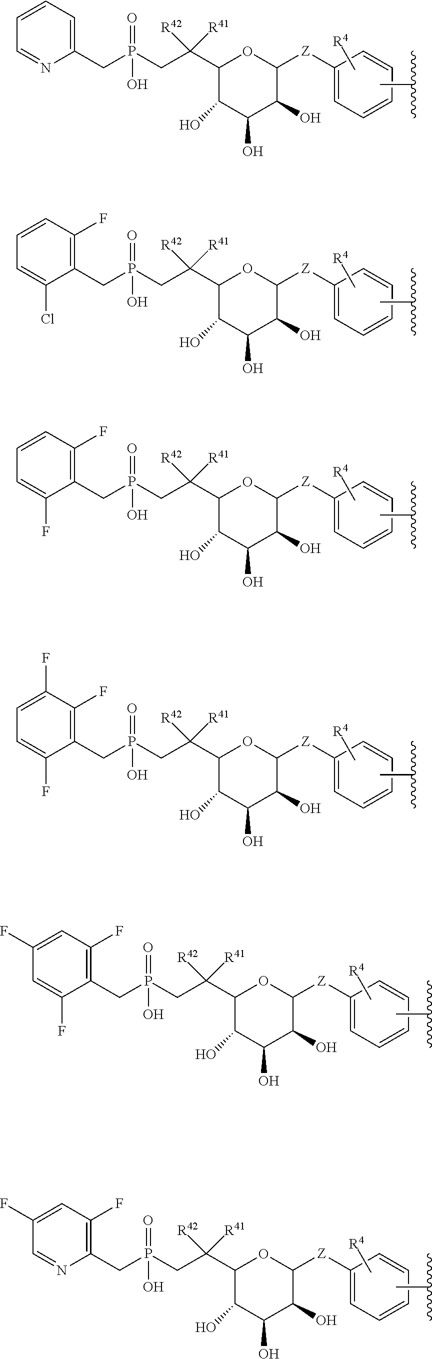

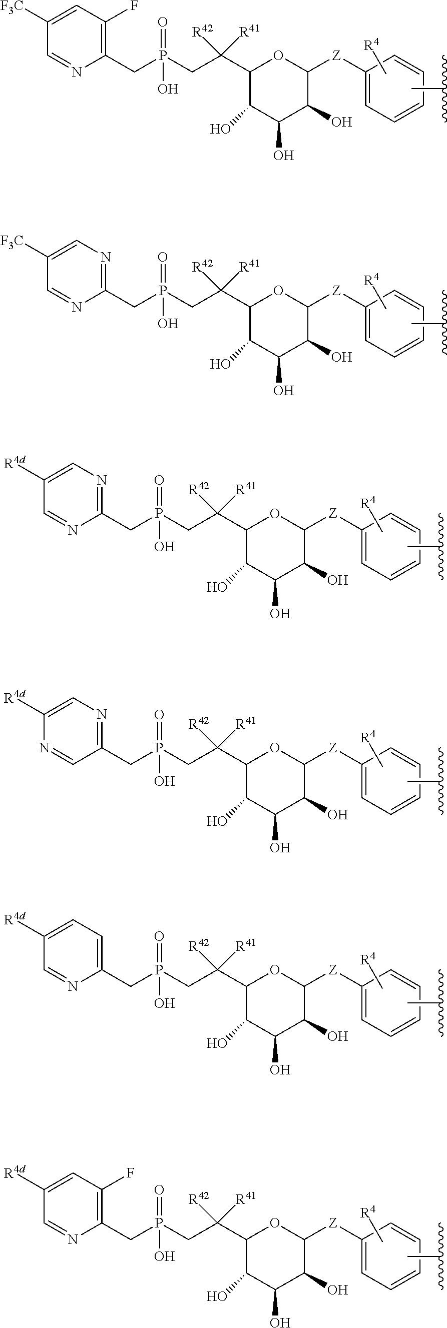

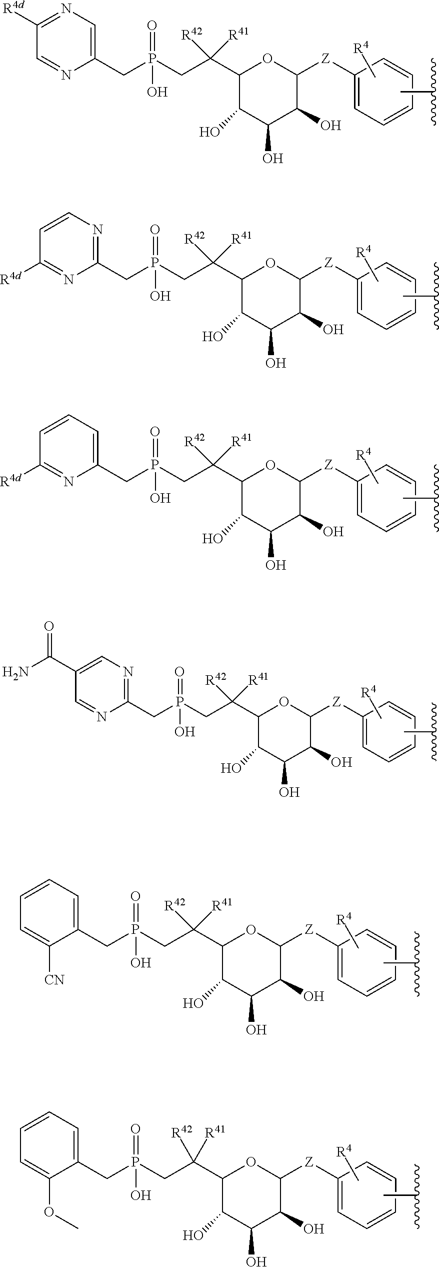

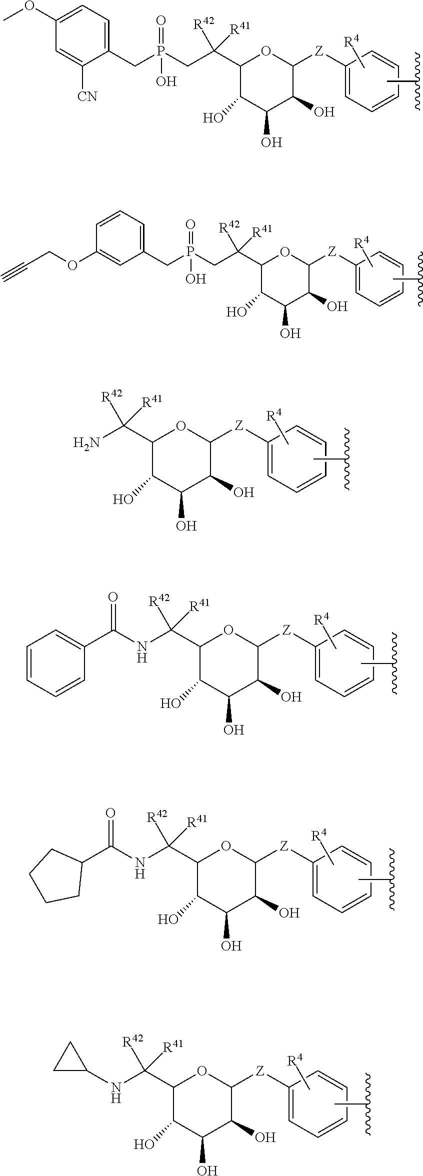

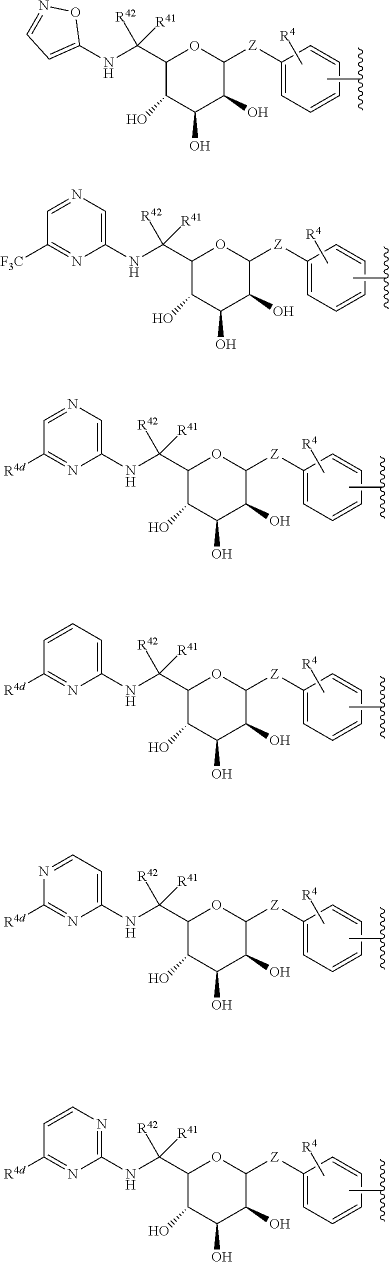

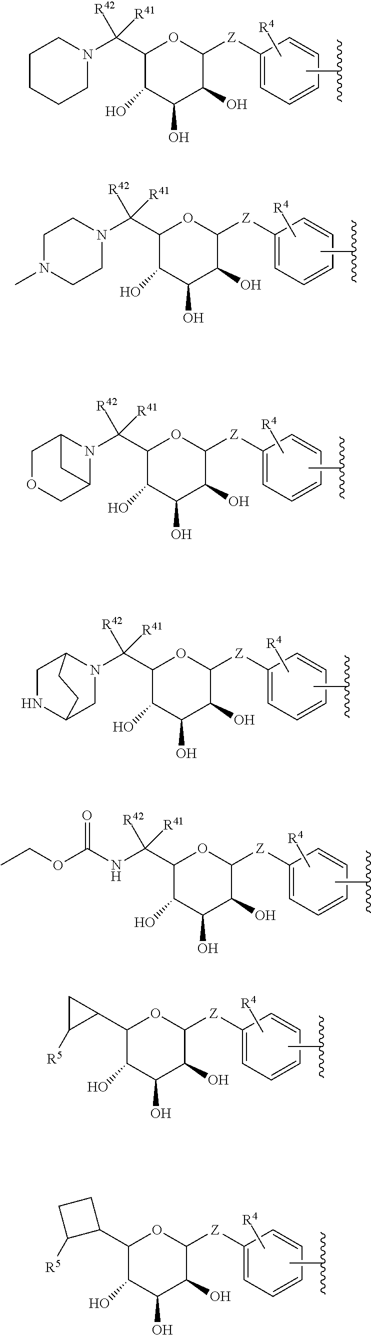

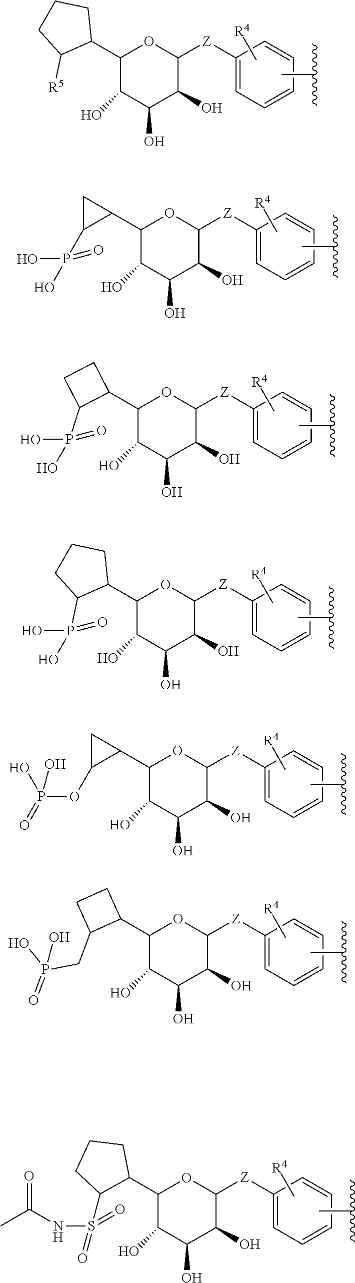

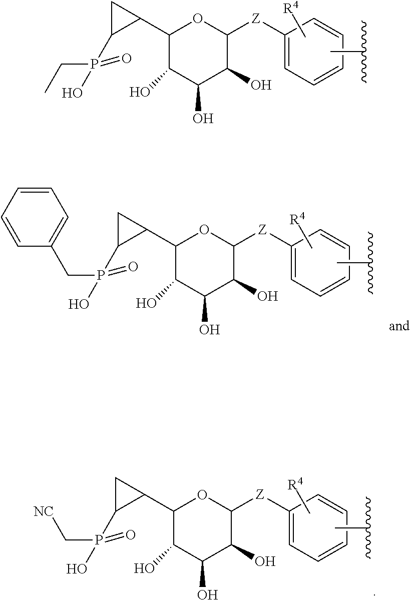

In certain embodiments, the Mannose 6-Phosphate Ligand is selected from:

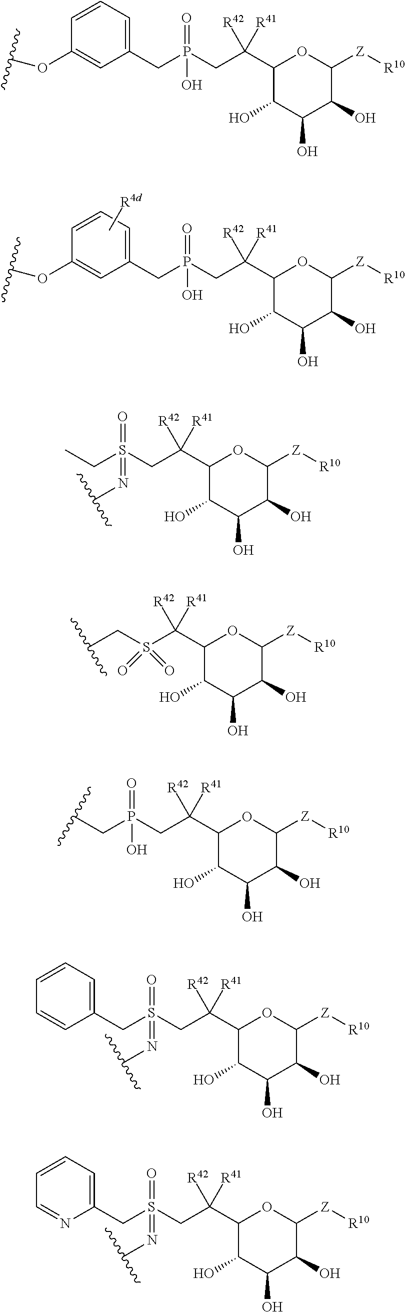

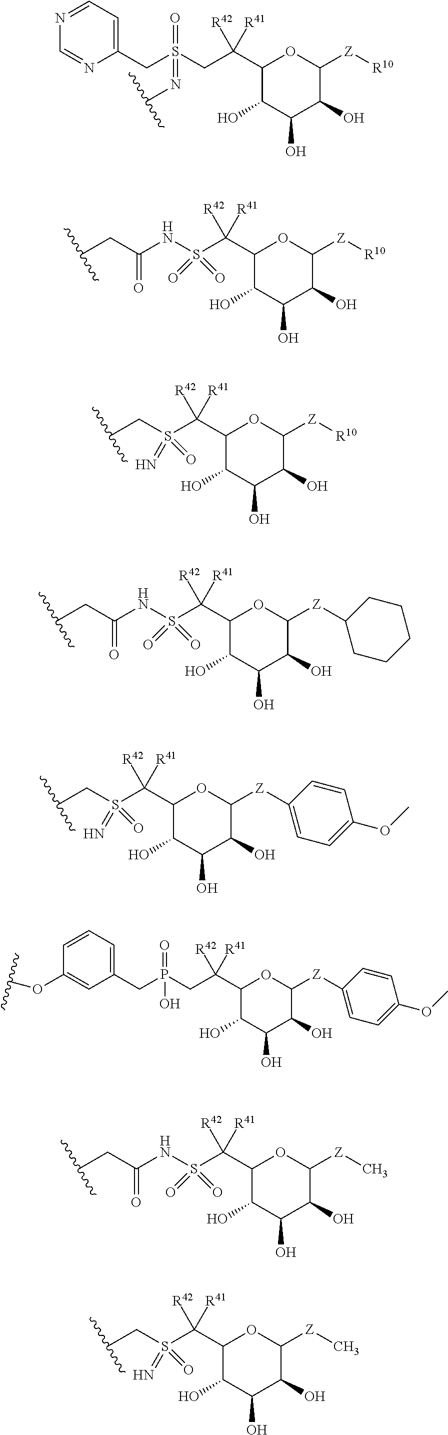

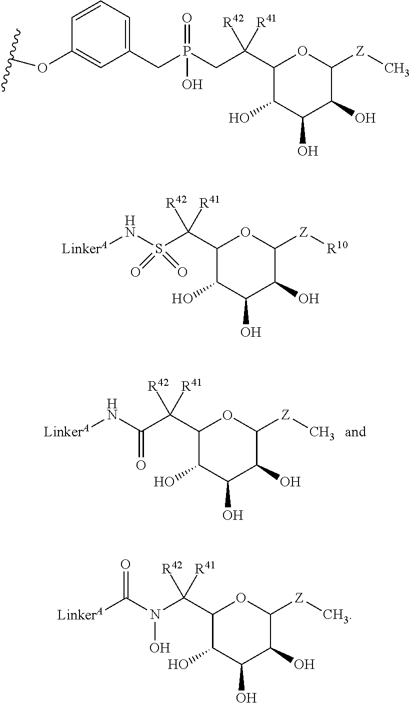

In certain embodiments, the compound of Formula IV is selected from:

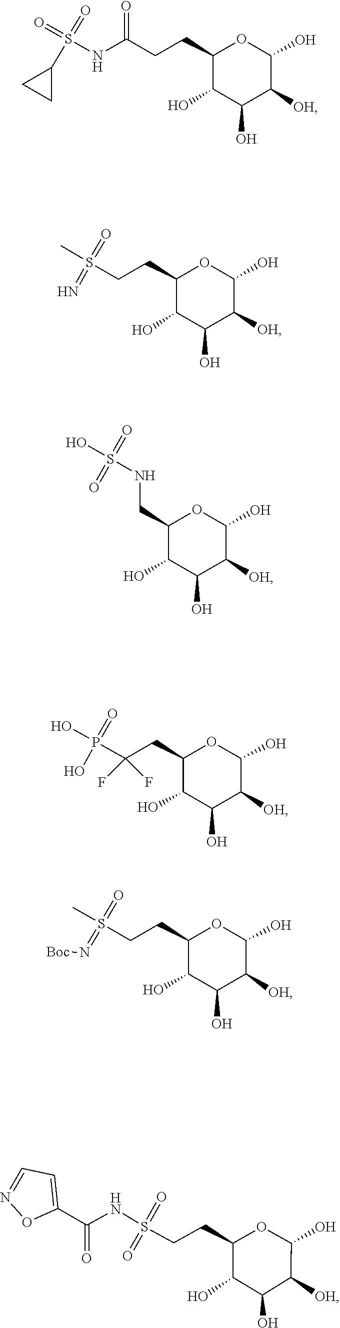 [SMILES: O=C(CC[C@H]1O[C@H](O)[C@@H](O)[C@@H](O)[C@@H]1O)NS(=O)(=O)C1CC1 ; CS(=N)(=O)CC[C@H]1O[C@H](O)[C@@H](O)[C@@H](O)[C@@H]1O ; O=S(=O)(O)NC[C@H]1O[C@H](O)[C@@H](O)[C@@H](O)[C@@H]1O ; O=P(O)(O)C(F)(F)C[C@H]1O[C@H](O)[C@@H](O)[C@@H](O)[C@@H]1O ; CC(C)(C)OC(=O)N=S(C)(=O)CC[C@H]1O[C@H](O)[C@@H](O)[C@@H](O)[C@@H]1O ; O=C(NS(=O)(=O)CC[C@H]1O[C@H](O)[C@@H](O)[C@@H](O)[C@@H]1O)c1ccno1]

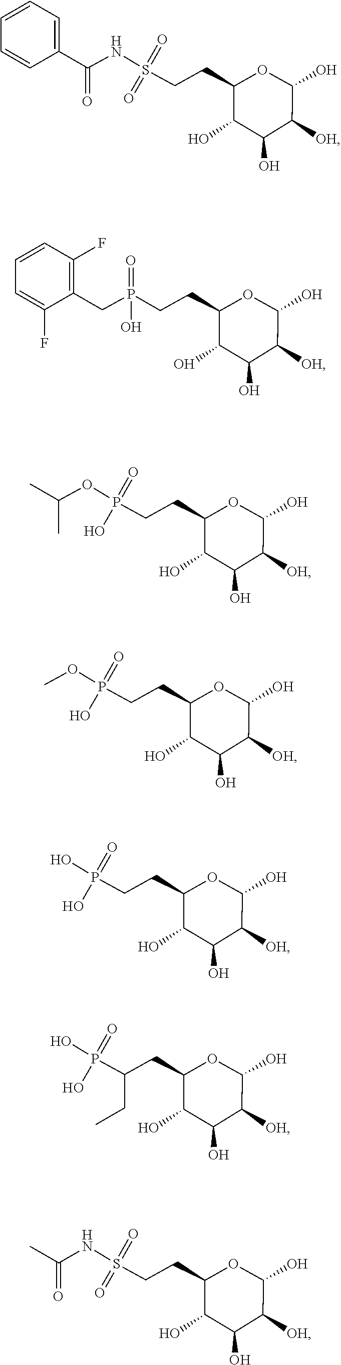

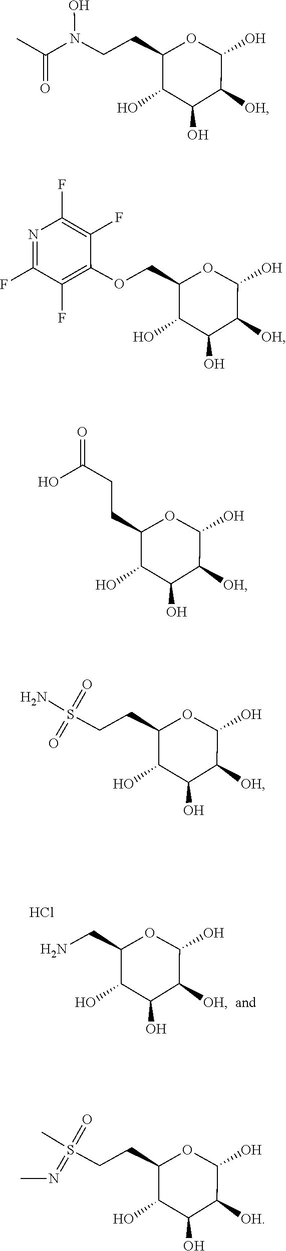

In certain embodiments, the compound of Formula IV is selected from:

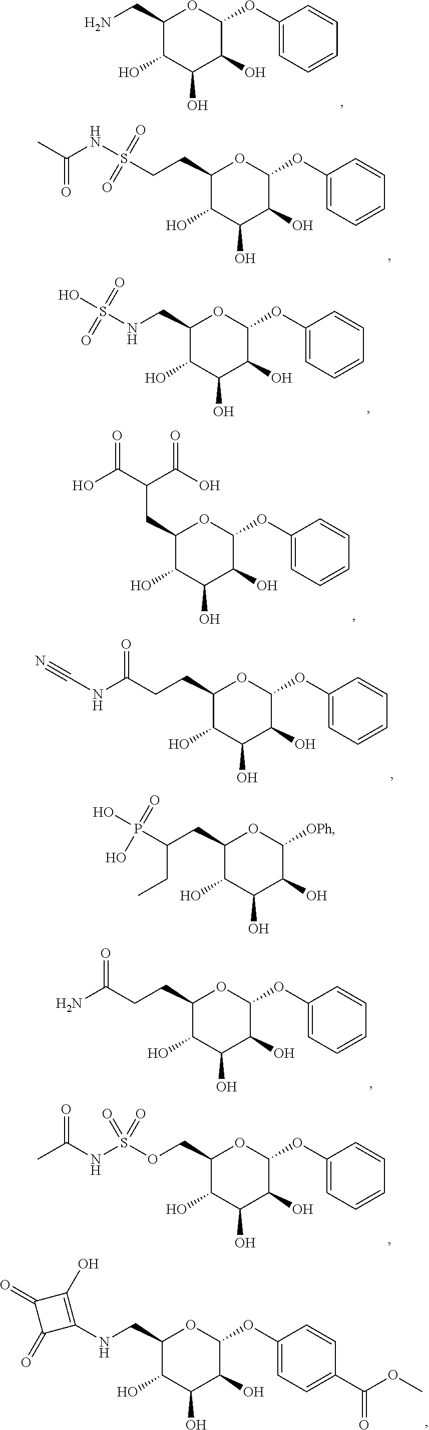

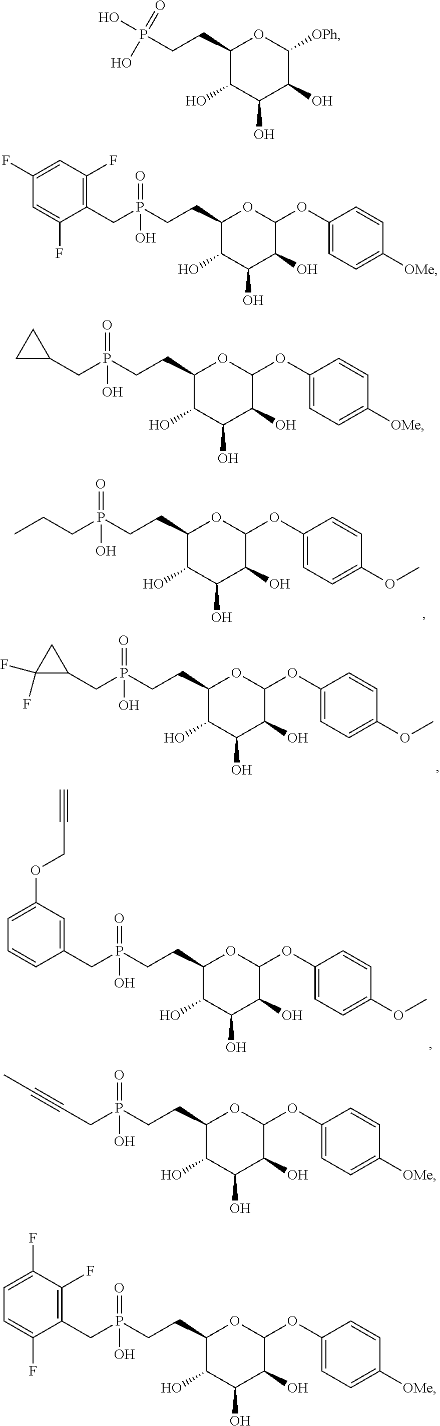

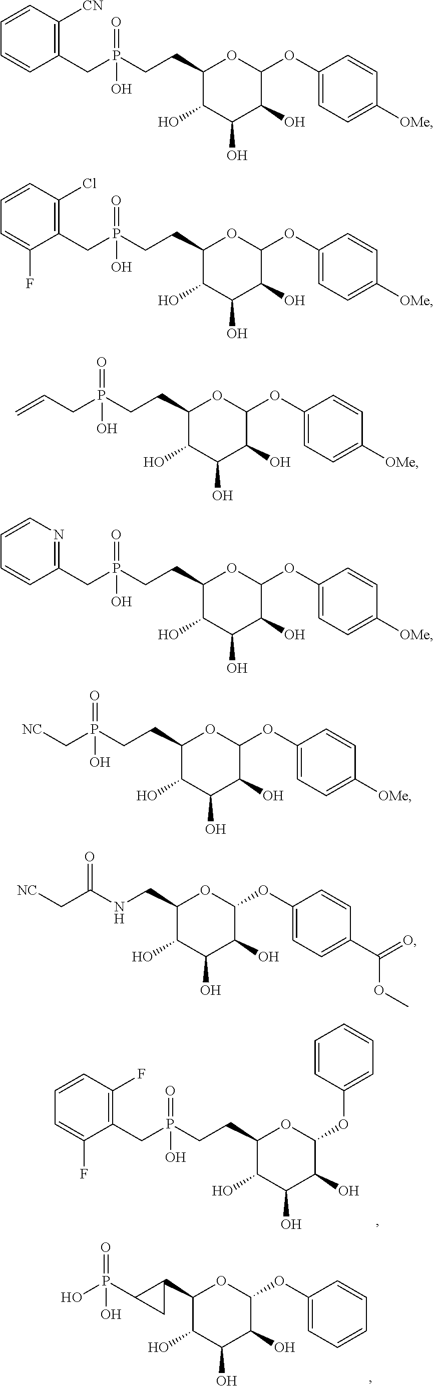

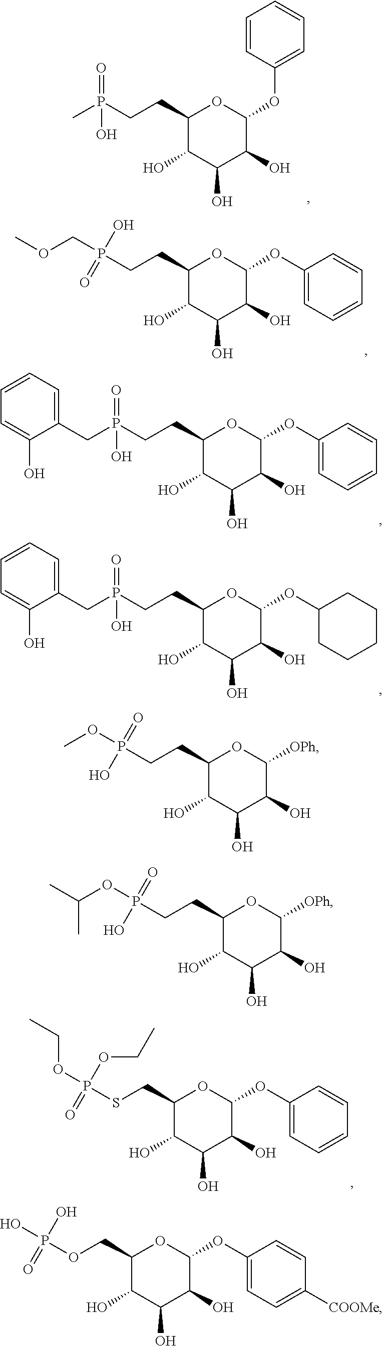

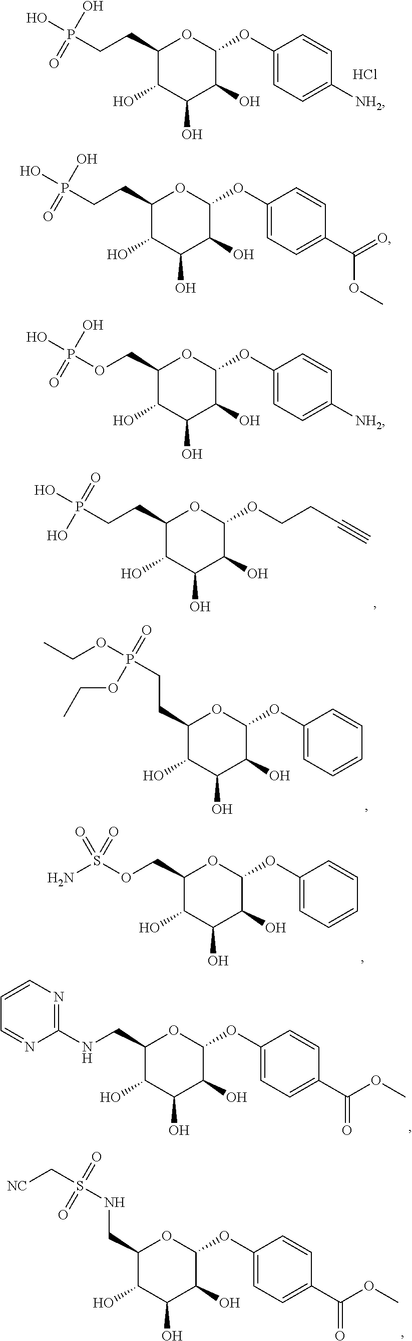

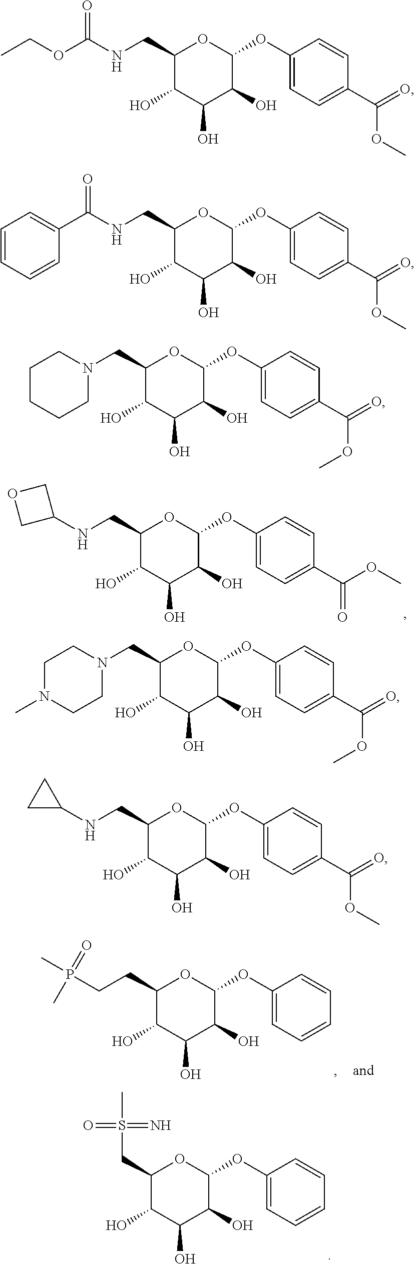

In certain embodiments, the compound of Formula IV is selected from:

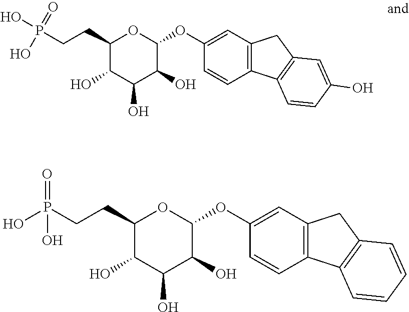 [SMILES: O=P(O)(O)CC[C@H]1O[C@H](Oc2ccc3c(c2)Cc2cc(O)ccc2-3)[C@@H](O)[C@@H](O)[C@@H]1O ; O=P(O)(O)CC[C@H]1O[C@H](Oc2ccc3c(c2)Cc2ccccc2-3)[C@@H](O)[C@@H](O)[C@@H]1O]

In certain embodiments, the compound of Formula IV is selected from:

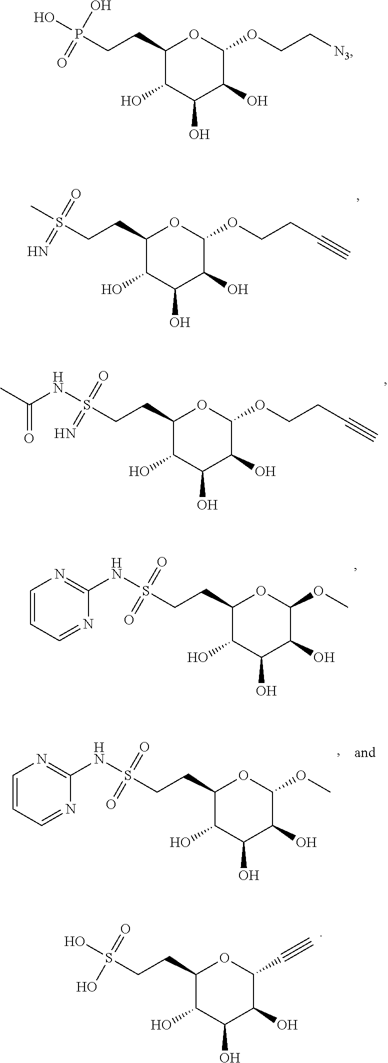

In certain embodiments, the compound of Formula IV is selected from

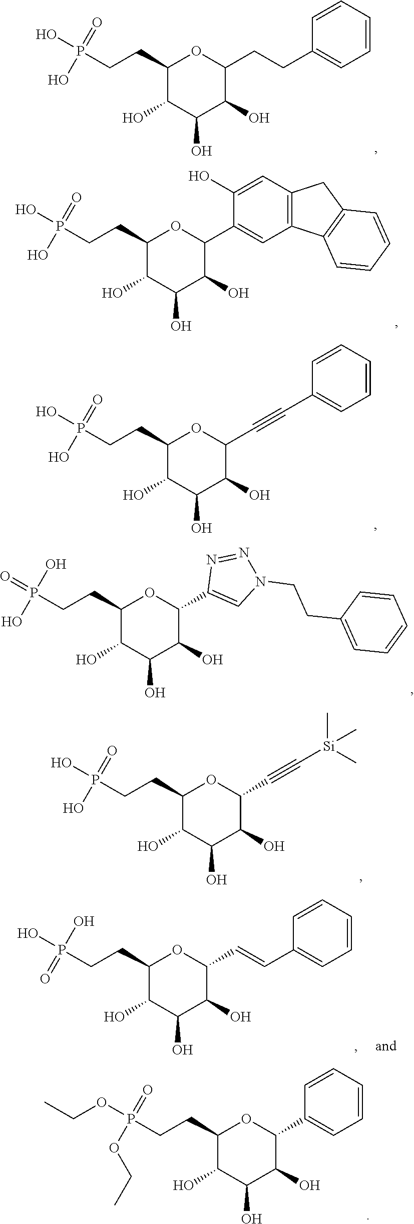

In certain embodiments, the compound of Formula IV is

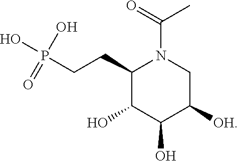 [SMILES: CC(=O)N1C[C@@H](O)[C@@H](O)[C@H](O)[C@H]1CCP(=O)(O)O]

Additional examples of M6PR ligands include:

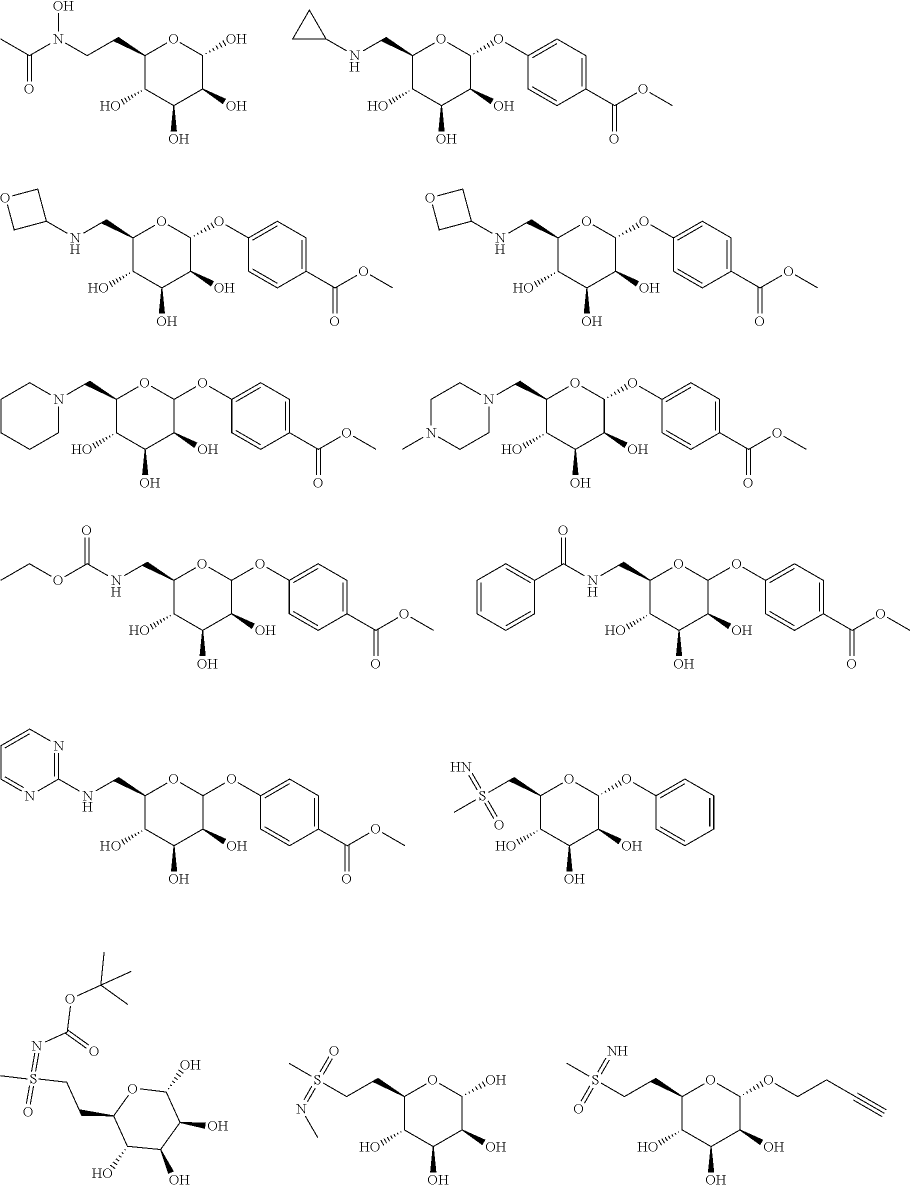

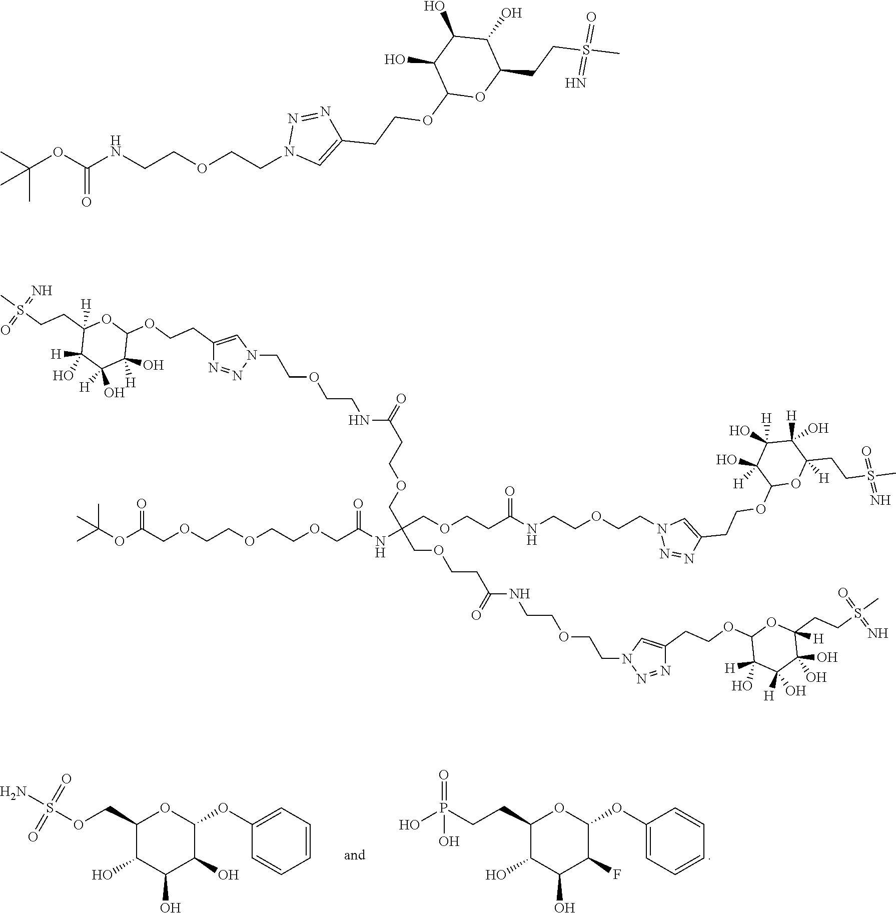

In certain embodiments, the compound of Formula IV is selected from

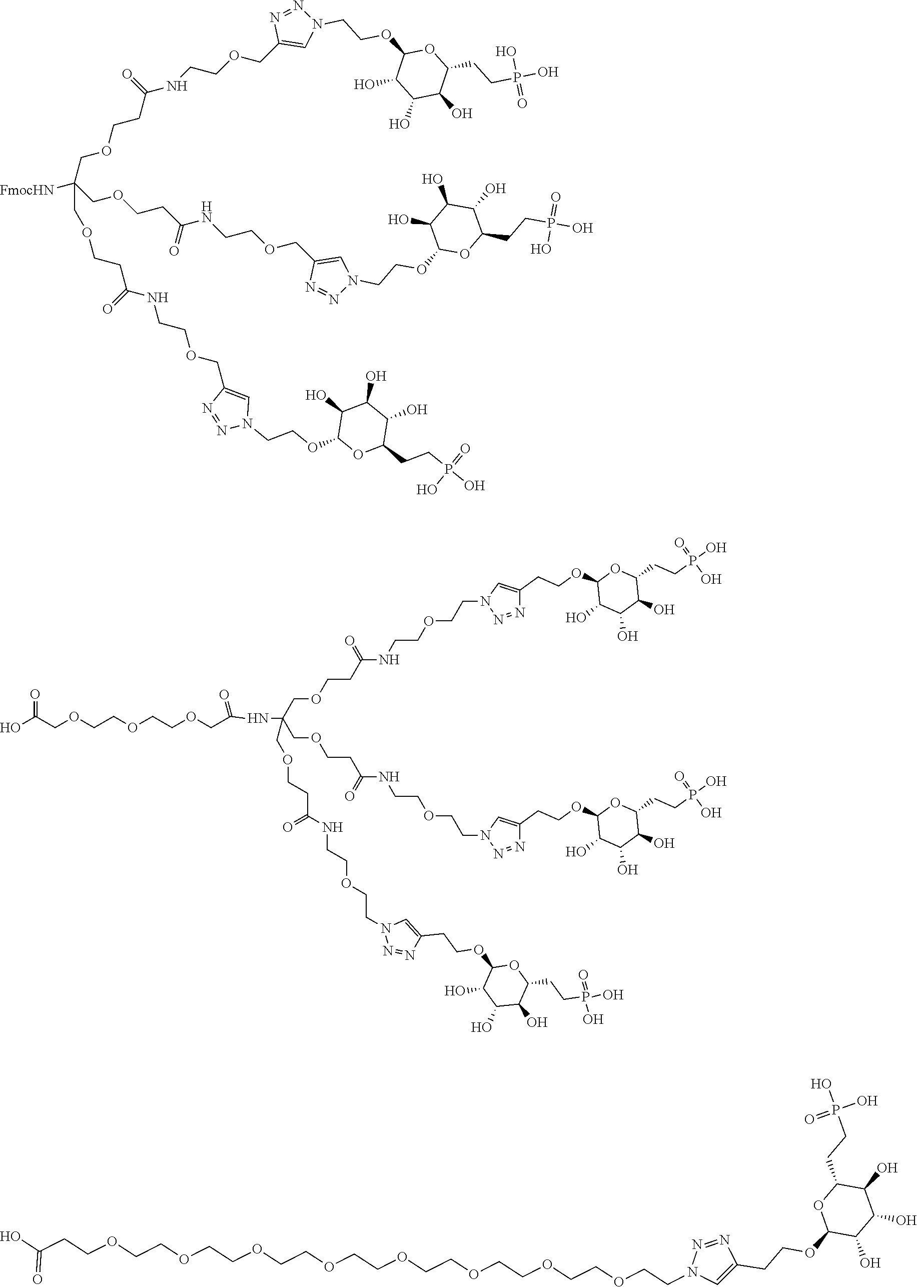

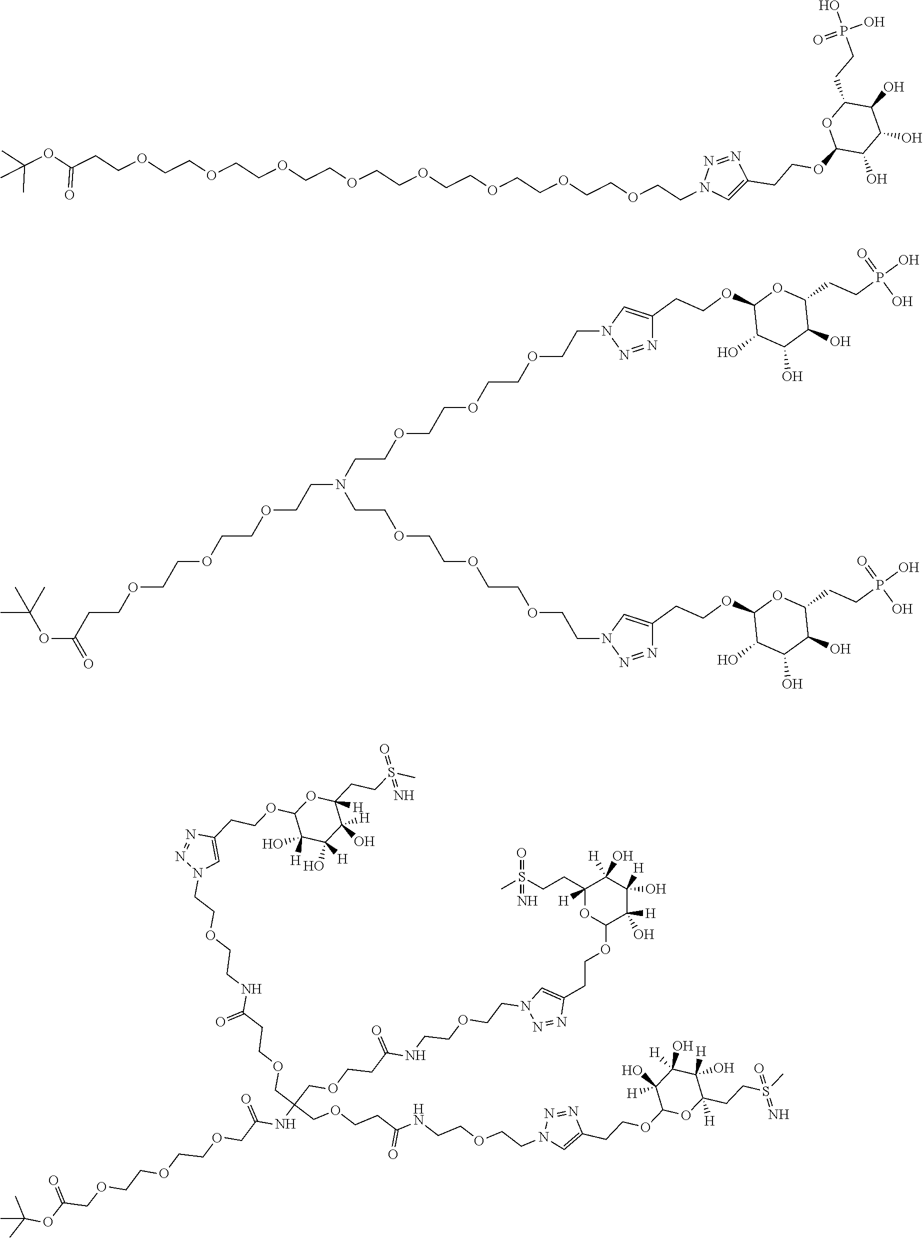

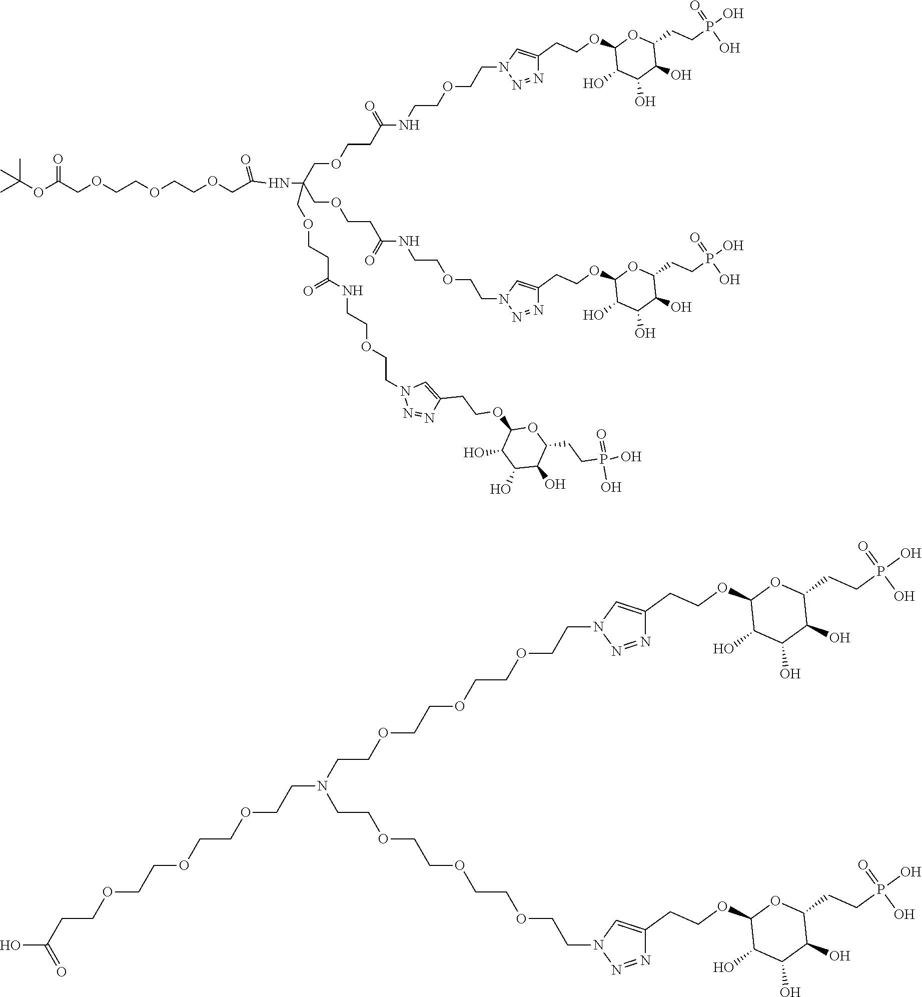 [SMILES: CC(C)(C)OC(=O)COCCOCCOCC(=O)NC(COCCC(=O)NCCOCCn1cc(CCO[C@H]2O[C@H](CCP(=O)(O)O)[C@@H](O)[C@H](O)[C@@H]2O)nn1)(COCCC(=O)NCCOCCn1cc(CCO[C@H]2O[C@H](CCP(=O)(O)O)[C@@H](O)[C@H](O)[C@@H]2O)nn1)COCCC(=O)NCCOCCn1cc(CCO[C@H]2O[C@H](CCP(=O)(O)O)[C@@H](O)[C@H](O)[C@@H]2O)nn1 ; O=C(O)CCOCCOCCOCCN(CCOCCOCCOCCn1cc(CCO[C@H]2O[C@H](CCP(=O)(O)O)[C@@H](O)[C@H](O)[C@@H]2O)nn1)CCOCCOCCOCCn1cc(CCO[C@H]2O[C@H](CCP(=O)(O)O)[C@@H](O)[C@H](O)[C@@H]2O)nn1]

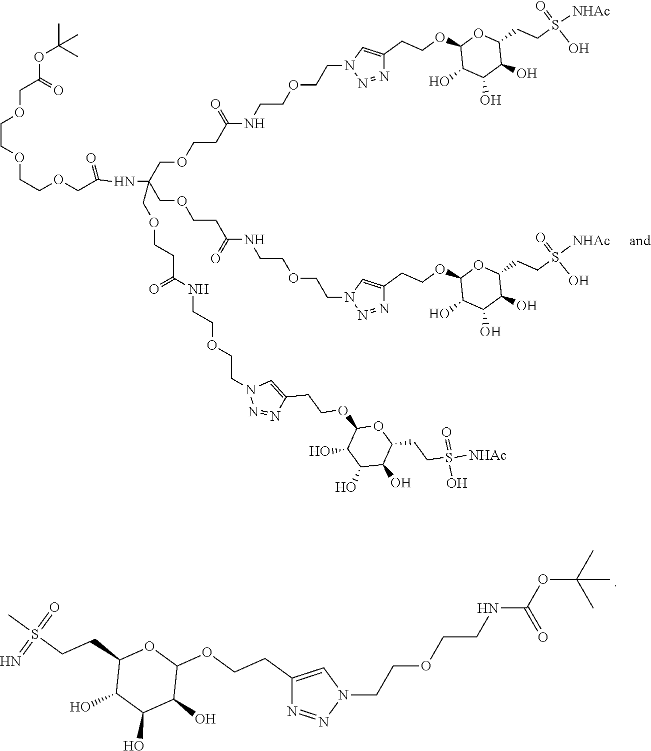

### IV. Non-Limiting Embodiments of the Present Invention

### Embodiments of \textcircled{A}

In certain embodiments,

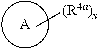

is cycloalkyl optionally substituted by 0, 1, 2, or 3 R^{4a} substituents.

In certain embodiments,

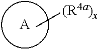

is heterocycle optionally substituted by 0, 1, 2, or 3 R^{4a} substituents.

In certain embodiments,

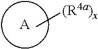

is aryl optionally substituted by 0, 1, 2, or 3 R^{4a} substituents.

In certain embodiments,

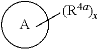

is heteroaryl optionally substituted by 0, 1, 2, or 3 R^{4a} substituents.

In certain embodiments,

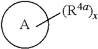

is phenyl optionally substituted by 0, 1, 2, or 3 R^{4a} substituents.

In certain embodiments,

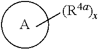

is pyridine optionally substituted by 0, 1, 2, or 3 R^{4a} substituents.

In certain embodiments,

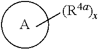

is pyrimidine optionally substituted by 0, 1, 2, or 3 R^{4a} substituents.

In certain embodiments,

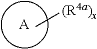

is pyrazine optionally substituted by 0, 1, 2, or 3 R^{4a} substituents.

In certain embodiments,

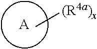

is pyrrole optionally substituted by 0, 1, or 2 R^{4a} substituents.

In certain embodiments,

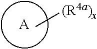

is pyrazole optionally substituted with an R^{4a}

In certain embodiments,

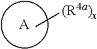

is imidazole optionally substituted with an R^{4a} substituent.

In certain embodiments,

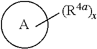

is isoxazole optionally substituted with an R^{4a} substituent.

In certain embodiments,

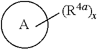

is oxazole optionally substituted with an R^{4a} substituent.

In certain embodiments,

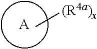

is cyclooctane optionally substituted by 0, 1, 2, or 3 R^{4a} substituents.

In certain embodiments,

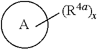

is cycloheptane optionally substituted by 0, 1, 2, or 3 R^{4a} substituents.

In certain embodiments,

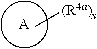

is cyclohexane optionally substituted by 0, 1, 2, or 3 R^{4a} substituents.

In certain embodiments,

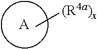

is cyclopentane optionally substituted by 0, 1, or 2 R^{4a} substituents.

In certain embodiments,

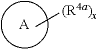

is cyclobutane.

In certain embodiments,

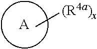

is piperidine optionally substituted by 0, 1, 2, or 3 R^{4a} substituents.

In certain embodiments,

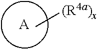

is piperazine optionally substituted by 0, 1, 2, or 3 R^{4a} substituents.

In certain embodiments,

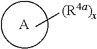

is morpholine optionally substituted by 0, 1, or 2 R^{4a} substituents.

In certain embodiments,

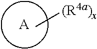

is pyrrolidine optionally substituted by 0, 1, or 2 R^{4a} substituents.

In certain embodiments x is 0.

In certain embodiments x is 1.

In certain embodiments x is 2.

### Embodiments of \textcircled{B}

In certain embodiments,

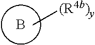

is cycloalkyl optionally substituted by 0, 1, 2, or 3 R^{4b} substituents.

In certain embodiments

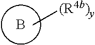

is heterocycle optionally substituted by 0, 1, 2, or 3 R^{4b} substituents.

In certain embodiments,

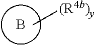

is cyclooctane optionally substituted by 0, 1, 2, or 3 R^{4b} substituents.

In certain embodiments,

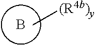

is cycloheptane optionally substituted by 0, 1, 2, or 3 R^{4b} substituents.

In certain embodiments,

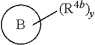

is cyclohexane optionally substituted by 0, 1, 2, or 3 R^{4b} substituents.

In certain embodiments,

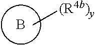

is cyclopentane optionally substituted by 0, 1, or 2 R^{4b} substituents.

In certain embodiments,

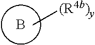

is cyclobutane.

In certain embodiments,

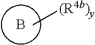

is piperidine optionally substituted by 0, 1, 2, or 3 R^{4b} substituents.

In certain embodiments,

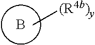

is piperazine optionally substituted by 0, 1, 2, or 3 R^{4b} substituents.

In certain embodiments,

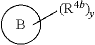

is morpholine optionally substituted by 0, 1, or 2 R^{4b} substituents.

In certain embodiments,

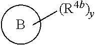

is pyrrolidine optionally substituted by 0, 1, or 2 R^{4b} substituents.

In certain embodiments y is 0.

In certain embodiments y is 1.

In certain embodiments y is 2.

### Embodiments of \textcircled{C}

In certain embodiments,

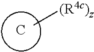

is aryl optionally substituted by 0, 1, 2, or 3 R^{4c} substituents.

In certain embodiments,

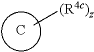

is heteroaryl optionally substituted by 0, 1, 2, or 3 R^{4c} substituents.

In certain embodiments,

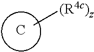

is phenyl optionally substituted by 0, 1, 2, or 3 R^{4c} substituents.

In certain embodiments,

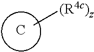

is pyridine optionally substituted by 0, 1, 2, or 3 R^{4c} substituents.

In certain embodiments,

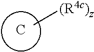

is pyrimidine optionally substituted by 0, 1, 2, or 3 R^{4c} substituents.

In certain embodiments,

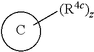

is pyrazine optionally substituted by 0, 1, 2, or 3 R^{4c} substituents.

In certain embodiments,

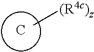

is pyrrole optionally substituted by 0, 1, or 2 R^{4c}

In certain embodiments,

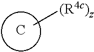

is pyrazole optionally substituted with an R^{4c} substituent.

In certain embodiments,

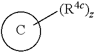

is imidazole optionally substituted with an R^{4c} substituent.

In certain embodiments,

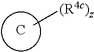

is isoxazole optionally substituted with an R^{4c} substituent.

In certain embodiments,

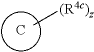

is oxazole optionally substituted with an R^{4c} substituent.

In certain embodiments z is 0.

In certain embodiments z is 1.

In certain embodiments z is 2.

### Embodiments of

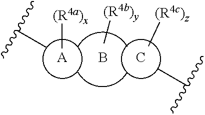

In certain embodiments,

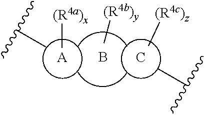

is selected from the group consisting of

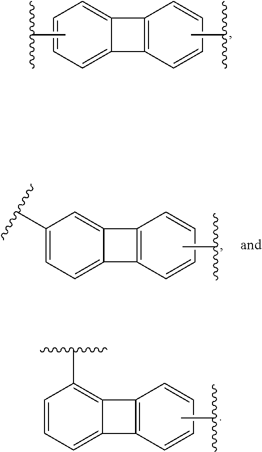

In certain embodiments,

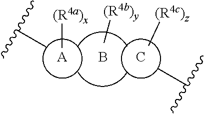

is selected from the group consisting of

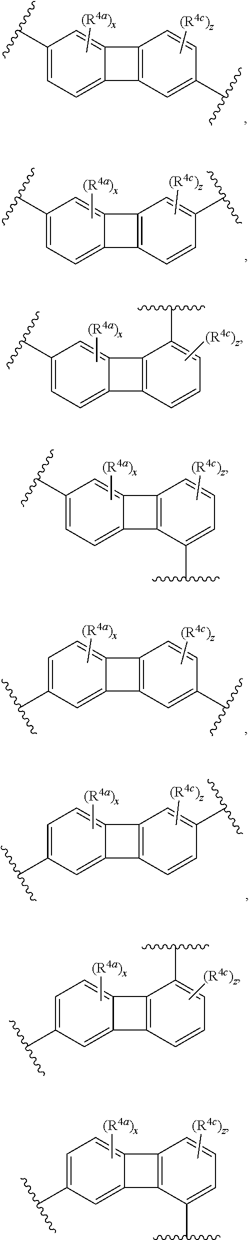

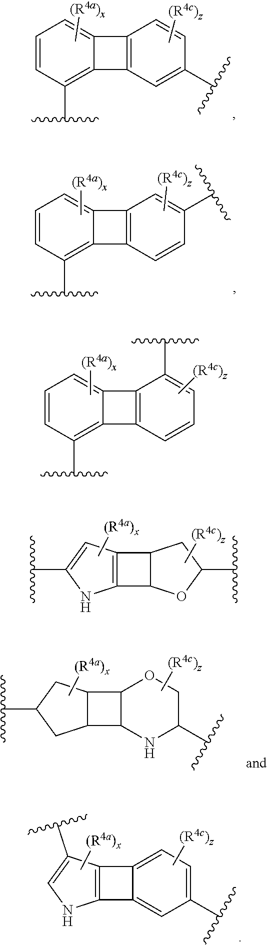

In certain embodiments,

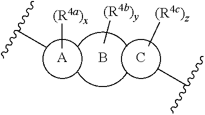

is selected from the group consisting of

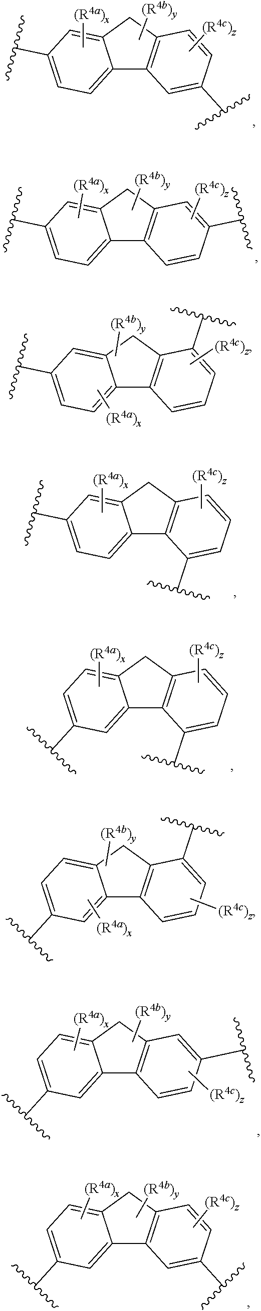

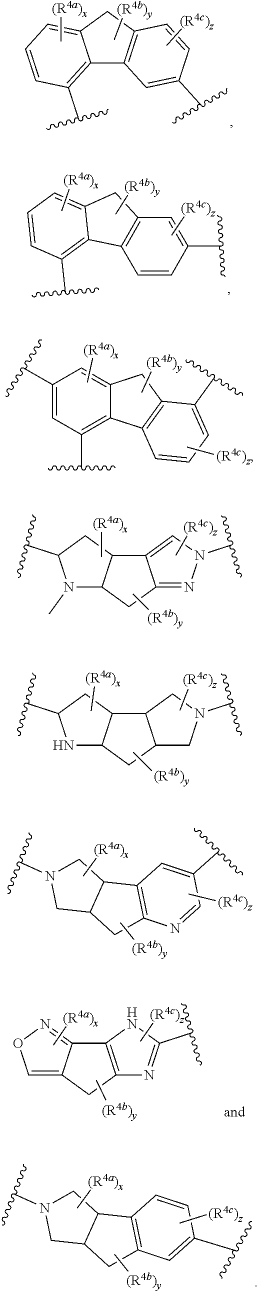

In certain embodiments,

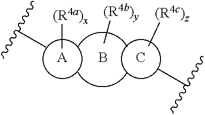

is selected from the group consisting of

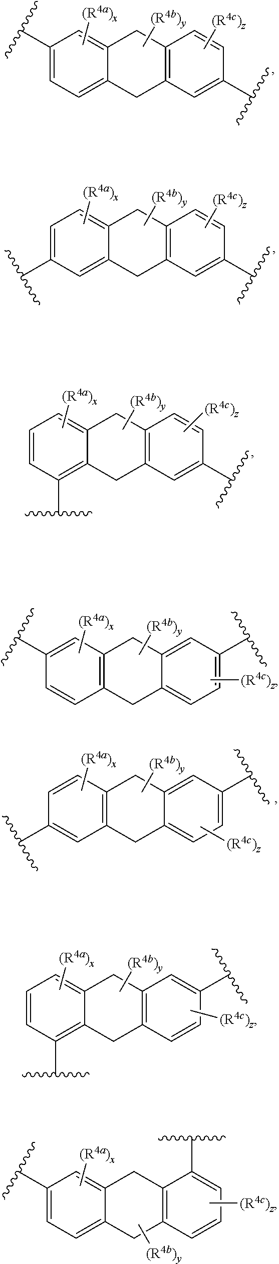

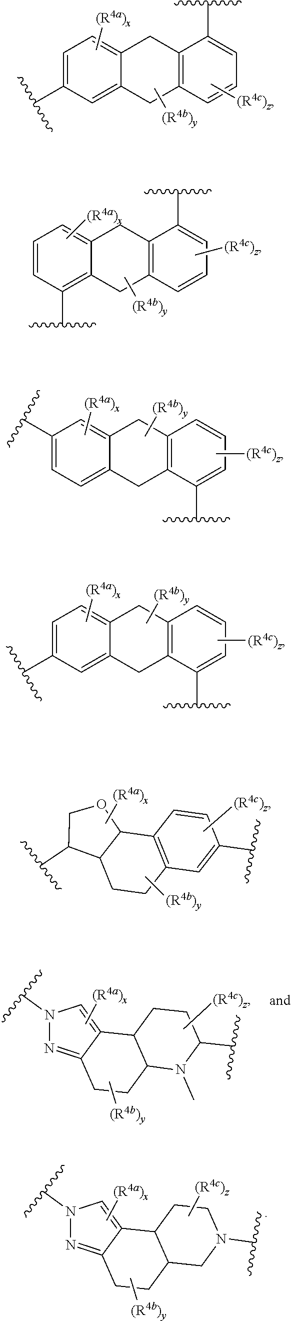

In certain embodiments,

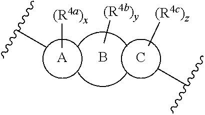

is selected from the group consisting of

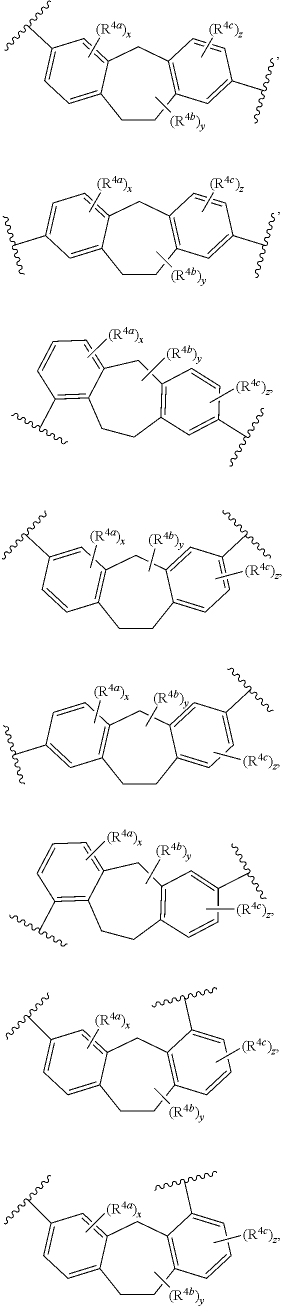

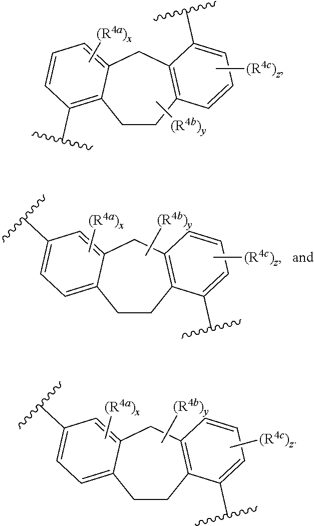

In certain embodiments,

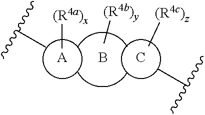

is selected from the group consisting of

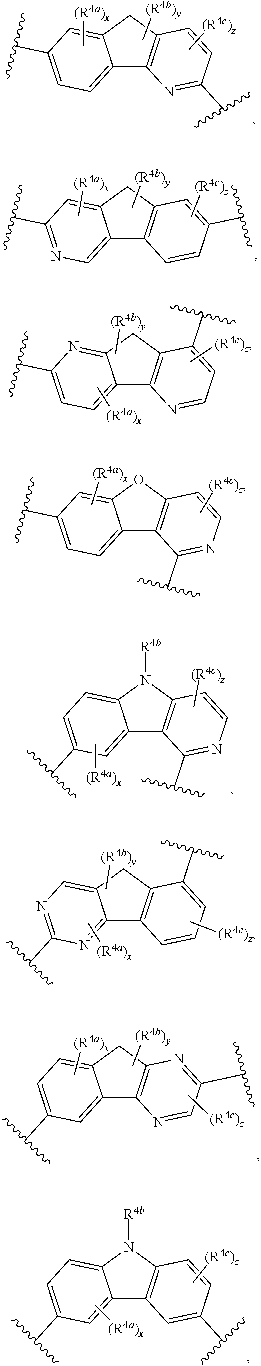

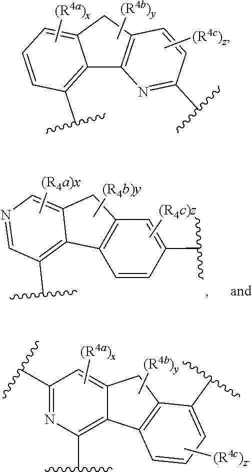

In certain embodiments,

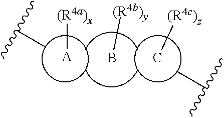

is selected from the group consisting of

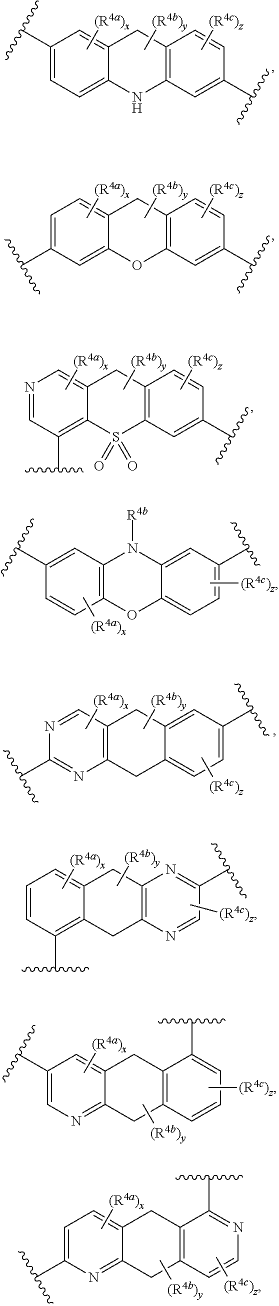

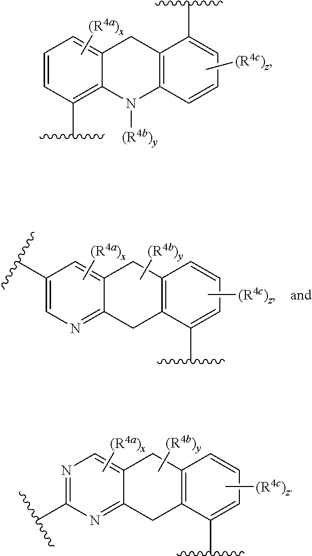

In certain embodiments,

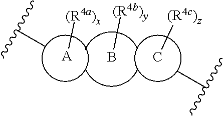

is selected from the group consisting of,

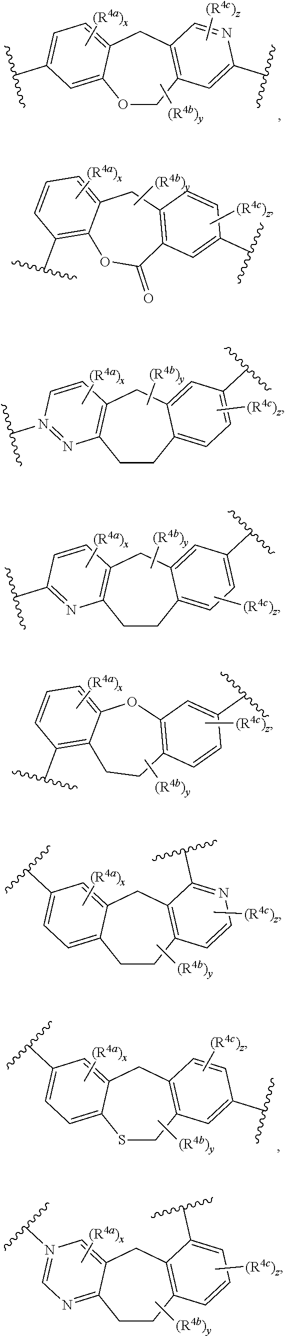

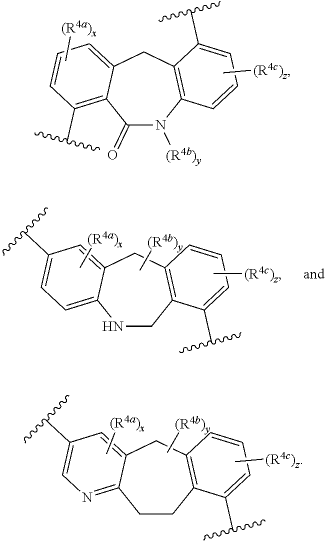

### Additional Embodiments of the Present Invention

1. A compound of Formula

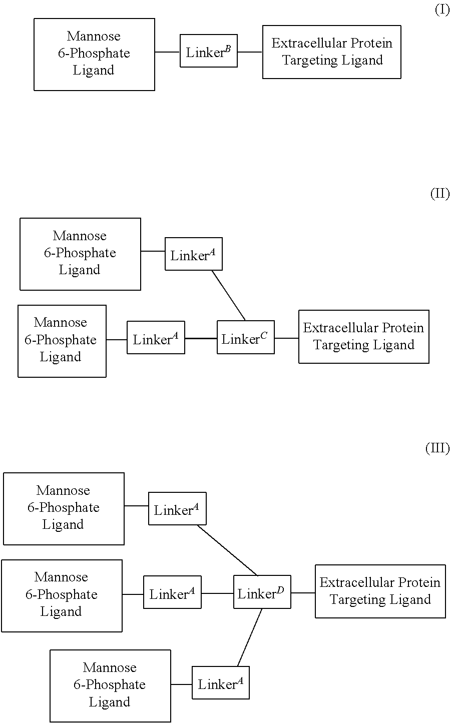

or a pharmaceutically acceptable salt thereof;
wherein:
Mannose 6-Phosphate Ligand is selected from:

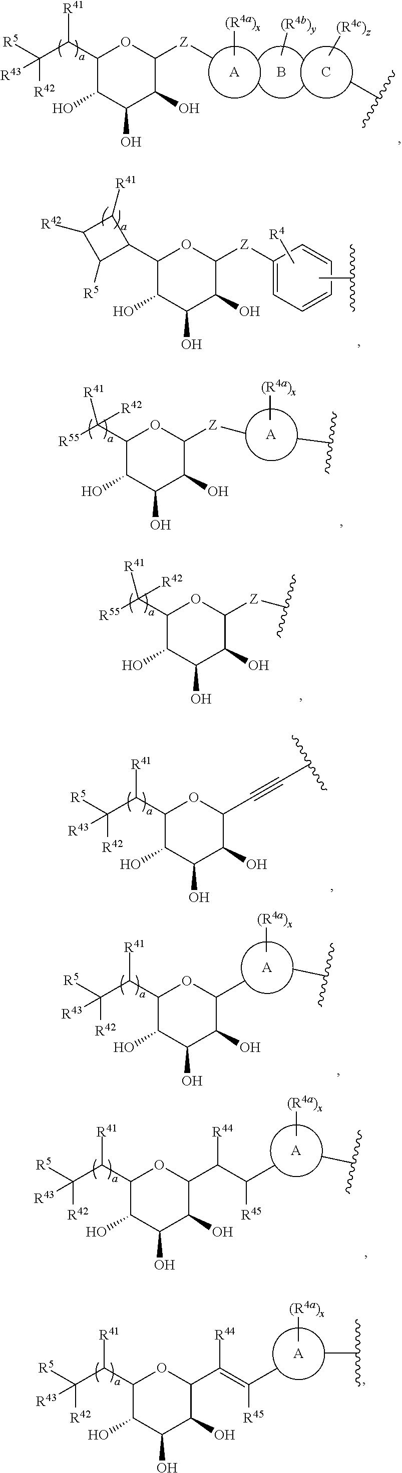

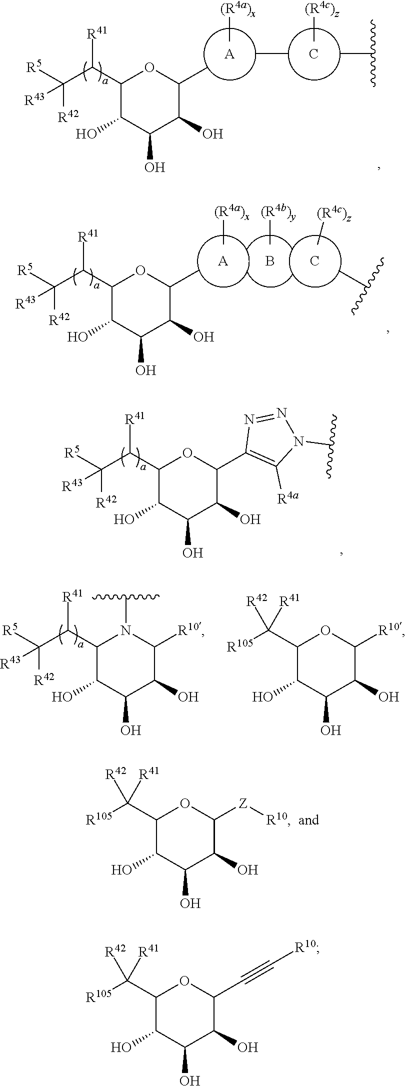

a, x, y, and z are independently 0, 1, 2, or 3;
Z is NR⁸, O, S, NC(O)R³, or CR⁴R⁸;
\textcircled{A} is cycloalkyl, heterocycle, aryl or heteroaryl;
\textcircled{B} is a nonaromatic cycloalkyl or heterocycle;
\textcircled{C} is aryl or heteroaryl;
R³ at each occurrence is independently selected from hydrogen, alkyl, haloalkyl, arylalkyl, heteroarylalkyl, alkenyl, alkynyl, aryl, heteroaryl, heterocycle, —OR⁸, and —NR⁸R⁹;
R⁴, R^{4a}, R^{4b}, and R⁴° C. are independently selected at each occurrence from hydrogen, F, Cl, Br, I, alkyl, haloalkyl, arylalkyl, heteroarylalkyl, alkenyl, alkynyl, aryl, heteroaryl, heterocycle, cyano, —OR⁶, —NR⁶R⁷, C(O)R³, S(O)R³, C(S)R³, and S(O)₂R³;
R⁴¹, R⁴², R⁴³, R⁴⁴, and R⁴⁵ are independently selected at each occurrence from hydrogen, alkyl, haloalkyl, F, arylalkyl, heteroarylalkyl, alkenyl, alkynyl, aryl, heteroaryl, and heterocycle;
R⁵⁰ is hydrogen, alkyl, haloalkyl, arylalkyl, heteroarylalkyl, alkenyl, alkynyl, aryl, heteroaryl, heterocycle, —OR⁶, or—NR⁶R⁷;
R⁵ is selected from

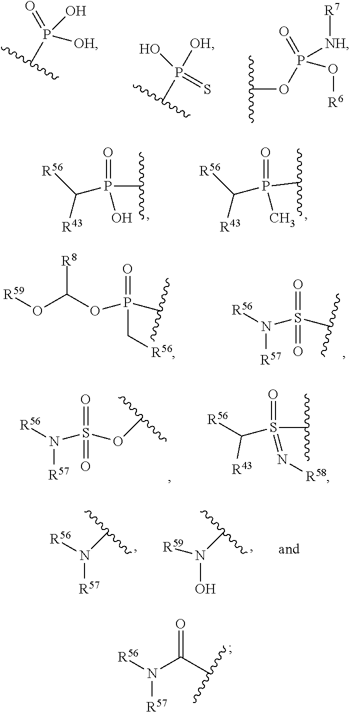

R⁵⁵ is selected from

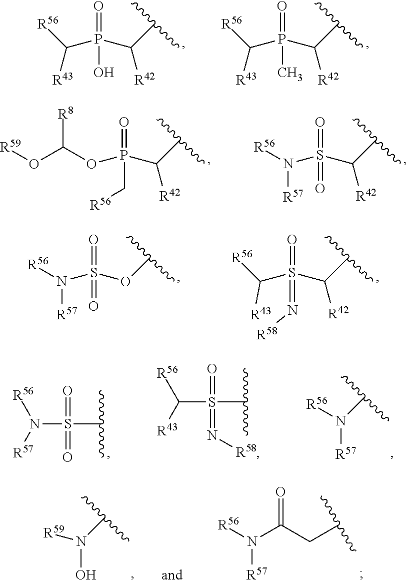

R⁵⁶ is independently selected from hydrogen, alkyl, alkenyl, alkynyl, haloalkyl, —O-alkyl, hydroxyl, cyano, heterocyclyl, aryl, and heteroaryl wherein each group except for hydrogen, hydroxyl, and cyano may optionally be substituted with 1, 2, or 3 independently selected R^{9a} substituents as allowed by valence;
R⁵⁷ is selected from hydrogen, alkyl, alkenyl, alkynyl, haloalkyl, —O-alkyl, aryl, heteroaryl, C(O) H, C(O) alkyl, C(O) alkenyl, C(O) alkynyl, C(O) haloalkyl, C(O) aryl, C(O)heteroaryl, C(O)O-alkyl, C(O)O-alkenyl, C(O)O-alkynyl, C(O)O-haloalkyl, C(O)O-aryl, C(O)O-heteroaryl and

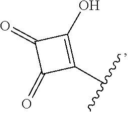 [SMILES: *c1c(O)c(=O)c1=O]

wherein each group except for hydrogen or C(O) H may optionally be substituted with 1, 2, or 3 independently selected Rob substituents as allowed by valence;
or R⁵⁶, R⁵⁷, and the nitrogen to which they are attached form a 5 to 7 membered heterocyclic ring which can be optionally substituted by alkyl, heterocycle, aryl, and heteroaryl;
R⁵⁸ is selected from hydrogen, alkyl, C(O) alkyl, C(O)O-alkyl, C(O) aryl, C(O)heteroaryl, C(O)O-aryl, C(O)O-arylalkyl, and C(O)O-heteroaryl, wherein each group may optionally be substituted with 1, 2, or 3 independently selected R^{9c} substituents as allowed by valence;
R⁵⁹ is selected from C(O) alkyl, C(O)O-alkyl, C(O) aryl, C(O)heteroaryl, C(O)O-aryl, and C(O)O-heteroaryl, wherein each group may optionally be substituted with 1, 2, or 3 independently selected R^{9d} substituents as allowed by valence;
R⁶ and R⁷ are independently selected at each occurrence from hydrogen, alkyl, arylalkyl, heteroarylalkyl, alkenyl, alkynyl, aryl, haloalkyl, heteroaryl, heterocycle, -alkyl—OR⁸, -alkyl—NR⁸R⁹, C(O)R³, S(O)R³, C(S)R³, and S(O)₂R³;
R⁸ is independently selected at each occurrence from hydrogen, alkyl, arylalkyl, heteroarylalkyl, alkenyl, alkynyl, aryl, heteroaryl, and heterocycle;
R⁹ is independently selected at each occurrence from hydrogen, alkyl, arylalkyl, heteroarylalkyl, alkenyl, alkynyl, aryl, heteroaryl, and heterocycle;
R^{9a}, R^{9b}, R^{9c}, and R^{9d} are independently selected at each occurrence from hydrogen, F, Cl, Br, I, alkyl, haloalkyl, arylalkyl, heteroarylalkyl, alkenyl, alkynyl, aryl, heteroaryl, heterocycle, cyano, —OR⁶, —NR⁶R⁷, C(O)R³, S(O)R³, C(S)R³, and S(O)₂R³;
R¹⁰ is selected from hydrogen, alkyl, aryl, heteroaryl, arylalkyl, heteroarylalkyl, alkyl-O-alkyl, C(O) aryl, C(O) alkyl, C(O) arylalkyl, C(O)heteroarylalkyl, each of which R¹⁰ except hydrogen is optionally substituted with 1, 2, 3, or 4 substituents selected from alkyl, alkenyl, alkynyl, haloalkyl, —OR⁶, F, Cl, Br, I, —NR⁶R⁷, heterocycle, heteroaryl, aryl, cyano, nitro, hydroxyl, amide, —SR³, —S(O)(NR⁶)R³, —NR⁸C(O)R³, —C(O)NR⁶R⁷, —C(O)OR³, and —C(O)R³; or R¹⁰ is

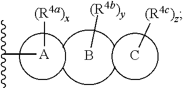

R¹⁰′ is selected from hydrogen, alkyl, aryl, heteroaryl, arylalkyl, heteroarylalkyl, each of which R¹⁰′ except hydrogen is optionally substituted with 1, 2, 3, or 4 substituents selected from alkyl, alkenyl, alkynyl, haloalkyl, —OR⁶, F, Cl, Br, I, —NR⁶R⁷, heterocycle, heteroaryl, aryl, cyano, nitro, hydroxyl, amide, —SR³, —S(O)(NR⁶)R³, —NR⁸C(O)R³, —C(O)NR⁶R⁷, —C(O)OR³, and —C(O)R³;
R¹⁰⁵ is selected from

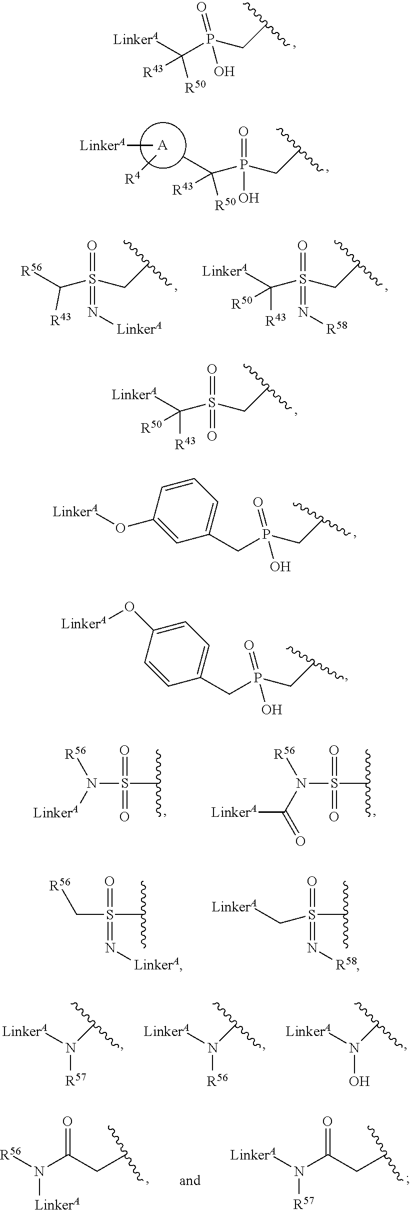

Linker^{A} and Linker^{B} are independently selected from:
wherein:

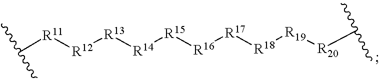

R¹¹, R¹², R¹³, R¹⁴, R¹⁵, R¹⁶, R¹⁷, R¹⁸, R¹⁹, and R²⁰ are independently at each occurrence selected from the group consisting of a bond, alkyl, —C(O)—, —C(O)O—, —OC(O)—, —SO₂—, —S(O)—, —C(S)—, —C(O)NR⁶—, —NR^{9c}(O)—, —C(O)N(OH)—, —N(OH)C(O)—, —O—, —S—, —NR⁶—, —C(R²¹R²¹)—, —S(O)(═N—R⁴⁴)—, —S(O)(═NR), —P(O)(R³)O—, —P(O)(R³)—, a divalent residue of a natural or unnatural amino acid, alkenyl, alkynyl, haloalkyl, alkoxy, aryl, heterocycle, heteroaryl, —CH₂CH₂—[O—(CH₂)₂]ₙ—O—, —CH₂CH₂—[O—(CH₂)₂]ₙ—NR⁶—, —CH₂CH₂—[O—(CH₂)₂]ₙ—, -[—(CH₂)₂—O-]ₙ-, —[O—(CH₂)₂]ₙ—, —[O—CH(CH₃)C(O)]ₙ-, —[C(O)—CH(CH₃)—O]ₙ-, —[O—CH₂C(O)]ₙ—, —[C(O)—CH₂—O]ₙ—, a divalent residue of a fatty acid, a divalent residue of an unsaturated or saturated mono- or di-carboxylic acid; each of which is optionally substituted with 1, 2, 3, or 4 substituents independently selected from R²¹;
Linker^{C} is selected from:

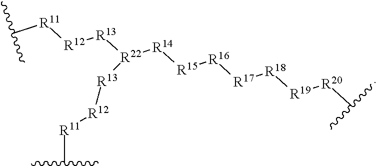

wherein:
R²² is independently at each occurrence selected from the group consisting of alkyl, —C(O)N—, —NC(O)—, —N—, —C(R²¹)—, —P(O)O—, —P(O)—, —P(O)(NR⁶R⁷)N—, alkenyl, haloalkyl, aryl, heterocycle, and heteroaryl, each of which is optionally substituted with 1, 2, 3, or 4 substituents independently selected from R²¹;
Linker^{D} is selected from:

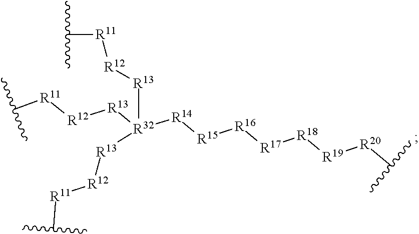

wherein

R³² is independently at each occurrence selected from the group consisting of alkyl, N⁺X⁻, —C—, alkenyl, haloalkyl, aryl, heterocycle, and heteroaryl, each of which except N⁺X⁻ and —C—is optionally substituted with 1, 2, 3, or 4 substituents independently selected from R²¹;
X⁻ is an anionic group, for example Br or Cl⁻;
n is independently selected at each instance from 0, 1, 2, 3, 4, 5, 6, 7, 8, 9, or 10;
R²¹ is independently at each occurrence selected from the group consisting of hydrogen, alkyl, alkenyl, alkynyl, F, Cl, Br, I, hydroxyl, alkoxy, azide, amino, cyano, —NR⁶R⁷, —NR⁸SO₂R³, —NR⁸S(O)R³, haloalkyl, aryl, heteroaryl, and heterocycle; and
Extracellular Protein Targeting Ligand is a chemical moiety that binds to a targeted disease-mediating extracellular protein.

2. The compound of embodiment 1, wherein the compound is of Formula:

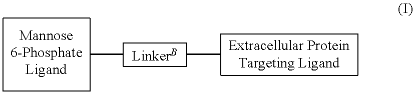

or a pharmaceutically acceptable salt thereof.

3. The compound of embodiment 1, wherein the compound is of Formula:

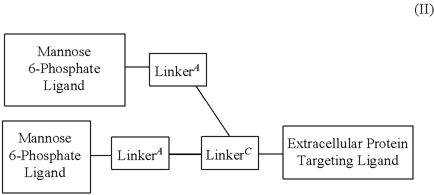

or a pharmaceutically acceptable salt thereof.

4. The compound of embodiment 1, wherein the compound is of Formula:

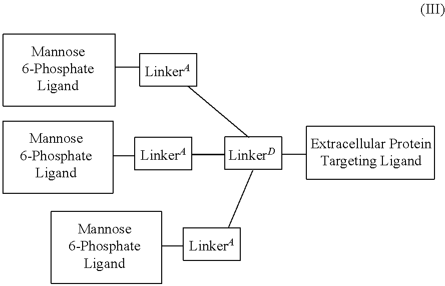

or a pharmaceutically acceptable salt thereof.

5. The compound of any one of embodiments 1-4, wherein Mannose 6-Phosphate Ligand is selected from:

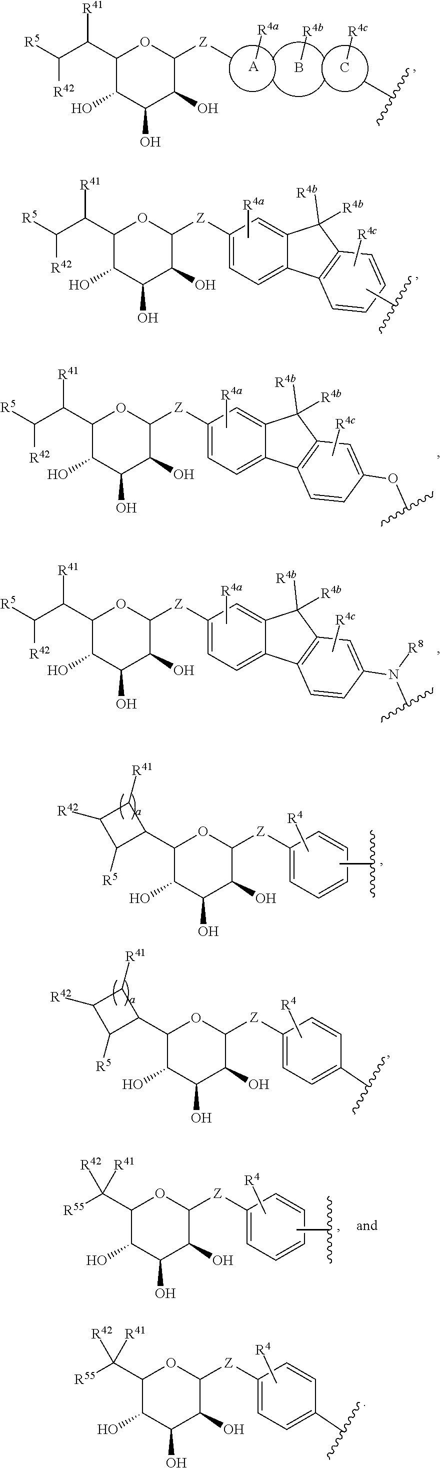

6. The compound of any one of embodiments 1-4, wherein Mannose 6-Phosphate Ligand

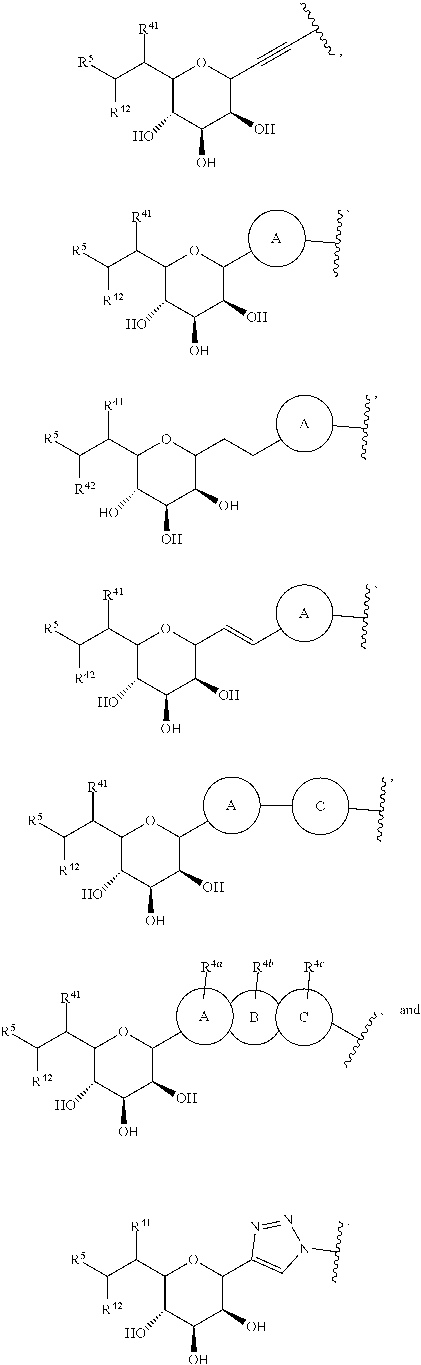

7. The compound of any one of embodiments 1-4, wherein Mannose 6-Phosphate Ligand is

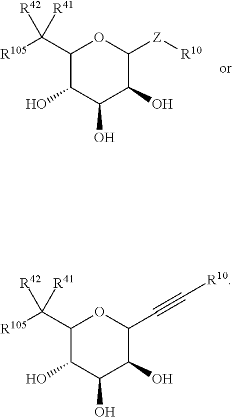

8. The compound of any one of embodiments 1-4, wherein Mannose 6-Phosphate Ligand is

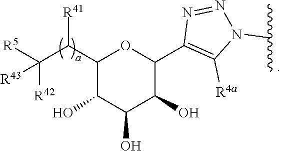

9. The compound of any one of embodiments 1-4, wherein Mannose 6-Phosphate Ligand is

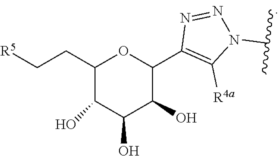

10. The compound of any one of embodiments 1-4, wherein Mannose 6-Phosphate Ligand is

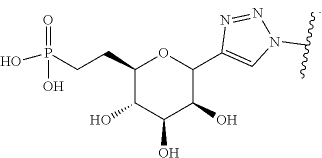 [SMILES: *n1cc(C2O[C@H](CCP(=O)(O)O)[C@@H](O)[C@H](O)[C@@H]2O)nn1]

11. The compound of any one of embodiments 1-10, wherein R⁴, R^{4a}, R^{4b}, and R⁴° C. are hydrogen.

12. The compound of any one of embodiments 1-10, wherein R⁴, R^{4a}, R^{4b}, or R^{4c} is selected from halogen, alkyl, haloalkyl, arylalkyl, heteroarylalkyl, alkenyl, alkynyl, aryl, heteroaryl, heterocycle, cyano, —OR⁶, —NR⁶R⁷, C(O)R³, S(O)R³, C(S)R³, and S(O)₂R³.

13. The compound of any one of embodiments 1-10, wherein R⁴, R^{4a}, R^{4b}, or R^{4c} is selected from halogen, alkyl, haloalkyl, aryl, heteroaryl, heterocycle, cyano, —OR⁶, —NR⁶R⁷, and C(O)R³.

14. The compound of any one of embodiments 1-10, wherein R⁴, R^{4a}, R^{4b}, or R^{4c} is halogen.

15. The compound of any one of embodiments 1-10, wherein R⁴, R^{4a}, R^{4b}, or R^{4c} is alkyl.

16. The compound of any one of embodiments 1-10, wherein R⁴, R^{4a}, R^{4b}, or R^{4c} is haloalkyl.

17. The compound of any one of embodiments 1-10, wherein R⁴, R^{4a}, R^{4b}, or R^{4c} is aryl.

18. The compound of any one of embodiments 1-10, wherein R⁴, R^{4a}, R^{4b}, or R^{4c} is heteroaryl.

19. The compound of any one of embodiments 1-10, wherein R⁴, R^{4a}, R^{4b}, or R^{4c} is cyano.

20. The compound of any one of embodiments 1-10, wherein R⁴, R^{4a}, R^{4b}, or R^{4c} is —OR⁶.

21. The compound of any one of embodiments 1-10, wherein R⁴, R^{4a}, R^{4b}, or R^{4c} is —NR⁶R⁷

22. The compound of any one of embodiments 1-21, wherein R⁵ is selected from:

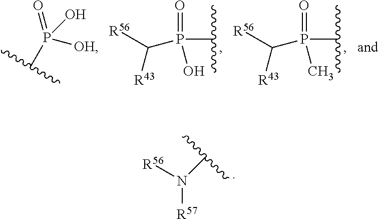

23. The compound of any one of embodiments 1-21, wherein R⁵⁵ is selected from:

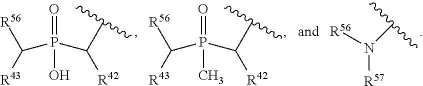

24. The compound of embodiment 22 or 23, wherein R⁵⁶ is hydrogen.

25. The compound of embodiment 22 or 23, wherein R⁵⁶ is aryl substituted 1, 2, or 3, R^{9a} substituents independently selected from halogen, alkyl, haloalkyl, arylalkyl, heteroarylalkyl, alkenyl, alkynyl, aryl, heteroaryl, heterocycle, cyano, —OR⁶, —NR⁶R⁷, C(O)R³, S(O)R³, C(S)R³, and S(O)₂R³.

26. The compound of embodiment 22 or 23, wherein R⁵⁶ is heteroaryl substituted 1, 2, or 3, R^{9a} substituents independently selected from halogen, alkyl, haloalkyl, arylalkyl, heteroarylalkyl, alkenyl, alkynyl, aryl, heteroaryl, heterocycle, cyano, —OR⁶, —NR⁶R⁷, C(O)R³, S(O)R³, C(S)R³, and S(O)₂R³.

27. The compound of embodiments 25 or 26, wherein R^{9a} is halogen.

28. The compound of embodiments 25 or 26, wherein R^{9a} is —OR⁶.

29. The compound of embodiment 22 or 23, wherein R⁵⁶ is cycloalkyl.

30. The compound of embodiment 22 or 23, wherein R⁵⁶ is halocycloalkyl.

31. The compound of embodiment 22 or 23, wherein R⁵⁶ is —O-alkyl.

32. The compound of embodiment 22 or 23, wherein R⁵⁶ is alkynyl.

33. The compound of embodiment 22 or 23, wherein R⁵⁶ is alkenyl.

34. The compound of embodiment 22 or 23, wherein R⁵⁶ is cyano.

35. The compound of any one of embodiments 22-34, wherein R⁵⁷ is alkyl.

36. The compound of any one of embodiments 1 to 35, wherein R⁶ and R⁷ are independently selected at each occurrence from hydrogen, alkyl, aryl, haloalkyl, heteroaryl, heterocycle, C(O)R³, S(O)R³, C(S)R³, and S(O)₂R³.

37. The compound of any one of embodiments 1 to 36, wherein R⁶ and R⁷ are independently selected at each occurrence from hydrogen, alkyl, and C(O)R³, S(O)R³, C(S)R³, and S(O)₂R³.

38. The compound of any one of embodiments 1 to 37, wherein at least one of R⁶ and R⁷ are hydrogen.

39. The compound of any one of embodiments 1 to 38, wherein R⁸ and R⁹ are independently selected at each occurrence from hydrogen and alkyl.

40. The compound of any one of embodiments 1, 2, and 5-39, wherein Linker^{B} is:

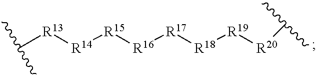

wherein:
R¹³, R¹⁴, R¹⁵, R¹⁶, R¹⁷, R¹⁸, R¹⁹, and R²⁰ are independently at each occurrence selected from the group consisting of a bond, alkyl, —C(O)—, —C(O)O—, —OC(O)—, —SO₂—, —S(O)—, —C(S)—, —C(O)NR⁶—, —NR⁶C(O)—, —O—, —S—, —NR⁶—, —P(O)(R³)O—, —P(O)(R³)—, alkenyl, alkynyl, haloalkyl, alkoxy, aryl, heterocycle, heteroaryl, —CH₂CH₂—[O—(CH₂)₂]ₙ—O—, —CH₂CH₂—[O—(CH₂)₂]ₙ—NR⁶—, —CH₂CH₂—[O—(CH₂)₂]ₙ—, -[—(CH₂)₂—O-]ₙ-, —[O—(CH₂)₂]ₙ—, —[O—CH(CH₃)C(O)]ₙ—, —[C(O)—CH(CH₃)—O]ₙ—, —[O—CH₂C(O)]ₙ—, —[C(O)—CH₂—O]ₙ—, or an amino acid; each of which is optionally substituted with 1 substituent independently selected from R²¹

41. The compound of embodiment 40, wherein R²¹ is independently at each occurrence selected from the group consisting of hydrogen, alkyl, alkenyl, alkynyl, F, Cl, Br, I, hydroxyl, alkoxy, azide, amino, cyano, —NR⁶R⁷, —NR⁸SO₂R³, —NR⁸S(O)R³, haloalkyl, aryl, heteroaryl, and heterocycle.

42. The compound of embodiment 40 or 41, wherein 1, 2, 3, or 4 of R¹³, R¹⁴, R¹⁵, R¹⁶, R¹⁷, R¹⁸, and R¹⁹ is bond.

43. The compound of embodiment 40 or 41, wherein 1, 2, 3, or 4 of R¹³, R¹⁴, R¹⁵, R¹⁶, R¹⁷, R¹⁸, and R¹⁹ is an amino acid.

44. The compound of embodiment 40, wherein Linker^{B} is selected from:

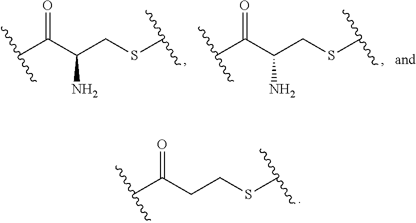 [SMILES: *SC[C@@H](N)C(*)=O ; *SC[C@H](N)C(*)=O ; *SCCC(*)=O]

45. The compound of embodiment 40, wherein Linker^{B} is selected from:

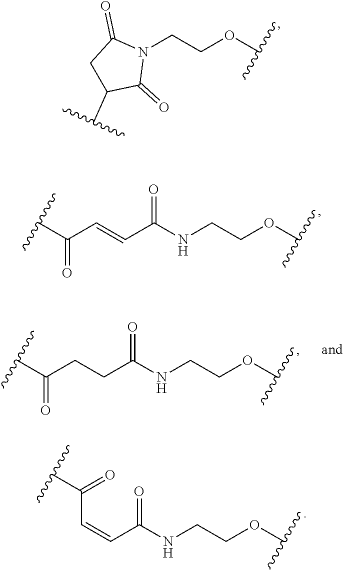 [SMILES: *OCCN1C(=O)CC(*)C1=O ; *OCCNC(=O)/C=C/C(*)=O ; *OCCNC(=O)CCC(*)=O ; *OCCNC(=O)/C=C\C(*)=O]

46. The compound of any one of embodiments 1, 3, and 5-39, wherein Linker^{C} is selected from:

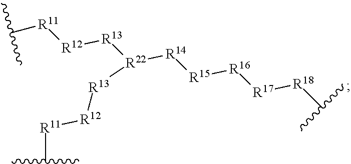

wherein:
R¹¹, R¹², R¹³, R¹⁴, R¹⁵, R¹⁶, R¹⁷, and R¹⁸ are independently at each occurrence selected from the group consisting of a bond, alkyl, —C(O)—, —C(O)O—, —OC(O)—, —SO₂—, —S(O)—, —C(S)—, —C(O)NR⁶—, —NR⁶C(O)—, —O—, —S—, —NR⁶—, —P(O)(R³)O—, —P(O)(R³)—, alkenyl, alkynyl, haloalkyl, alkoxy, aryl, heterocycle, heteroaryl, —CH₂CH₂—[O—(CH₂)₂]ₙ—O—, —CH₂CH₂—[O—(CH₂)₂]ₙ—NR⁶—, —CH₂CH₂—[O—(CH₂)₂]ₙ—, -[—(CH₂)₂—O-]ₙ-, —[O—(CH₂)₂]ₙ—, —[O—CH(CH₃)C(O)]ₙ—, —[C(O)—CH(CH₃)—O]ₙ—, —[O—CH₂C(O)]ₙ-, —[C(O)—CH₂—O]ₙ-, or an amino acid; each of which is optionally substituted with 1 substituent independently selected from R²¹

47. The compound of embodiment 46, wherein R²¹ is independently at each occurrence selected from the group consisting of hydrogen, alkyl, alkenyl, alkynyl, F, Cl, Br, I, hydroxyl, alkoxy, azide, amino, cyano, —NR⁶R⁷, —NR⁸SO₂R³, —NR⁸S(O)R³, haloalkyl, aryl, heteroaryl, and heterocycle.

48. The compound of embodiment 46 or 47, wherein Linker^{C} is selected from:

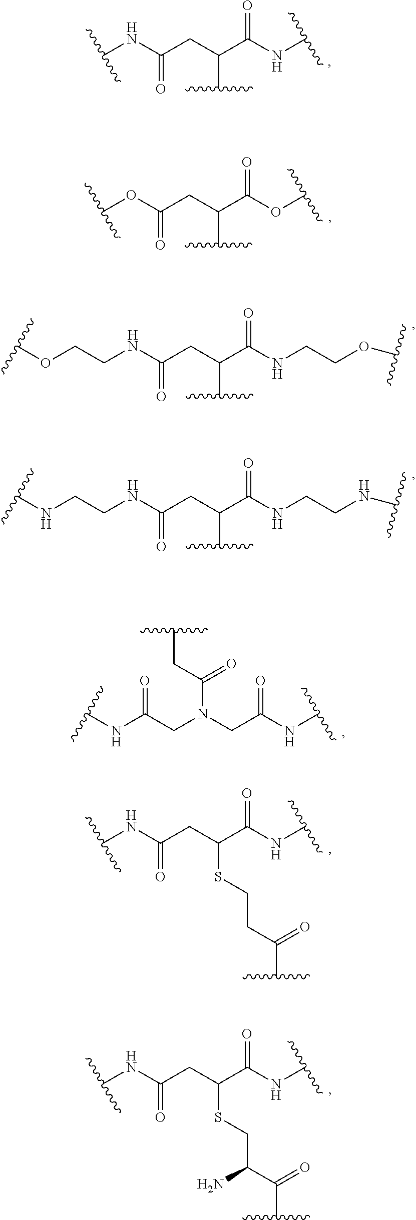

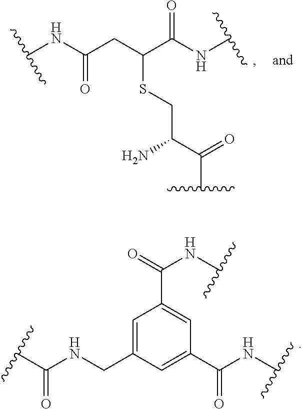 [SMILES: *NC(=O)CC(SC[C@@H](N)C(*)=O)C(=O)N* ; *NC(=O)c1cc(CNC(*)=O)cc(C(=O)N*)c1]

49. The compound of any one of embodiments 1, 4, and 539, wherein Linker^{D} is selected from:

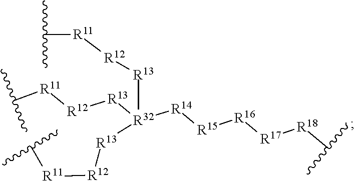

wherein:
R¹¹, R¹², R¹³, R¹⁴, R¹⁵, R¹⁶, R¹⁷, and R¹⁸ are independently at each occurrence selected from the group consisting of a bond, alkyl, —C(O)—, —C(O)O—, —OC(O)—, —SO₂—, —S(O)—, —C(S)—, —C(O)NR⁶—, —NR^{9c}(O)—, —O—, —S—, —NR⁶—, —P(O)(R³)O—, —P(O)(R³)—, alkenyl, alkynyl, haloalkyl, alkoxy, aryl, heterocycle, heteroaryl, —CH₂CH₂—[O—(CH₂)₂]ₙ—O—, —CH₂CH₂—[O—(CH₂)₂]ₙ—NR⁶—, —CH₂CH₂—[O—(CH₂)₂]ₙ—, -[—(CH₂)₂—O-]ₙ-, —[O—(CH₂)₂]ₙ—, —[O—CH(CH₃)C(O)]ₙ—, —[C(O)—CH(CH₃)—O]ₙ—, —[O—CH₂C(O)]ₙ—, —[C(O)—CH₂—O]ₙ—, or an amino acid; each of which is optionally substituted with 1 substituent independently selected from R²¹

50. The compound of any one of embodiments 1 to 49, wherein the Extracellular Protein Targeting Ligand targets an immunoglobin.

51. The compound of any one of embodiments 1 to 50, wherein the Extracellular Protein Targeting Ligand targets IgA.

52. The compound of embodiment 51, wherein the Extracellular Protein Targeting Ligand is:

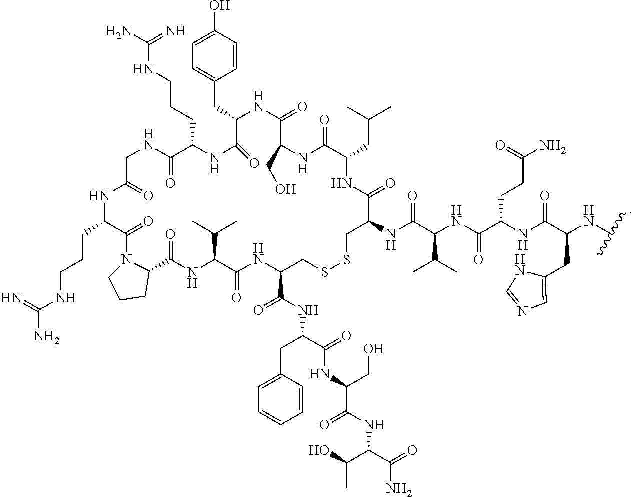 [SMILES: *N[C@@H](Cc1cnc[nH]1)C(=O)N[C@@H](CCC(N)=O)C(=O)N[C@H](C(=O)N[C@H]1CSSC[C@@H](C(=O)N[C@@H](Cc2ccccc2)C(=O)N[C@@H](CO)C(=O)N[C@H](C(N)=O)[C@@H](C)O)NC(=O)[C@H](C(C)C)NC(=O)[C@@H]2CCCN2C(=O)[C@H](CCCNC(=N)N)NC(=O)CNC(=O)[C@H](CCCNC(=N)N)NC(=O)[C@H](Cc2ccc(O)cc2)NC(=O)[C@H](CO)NC(=O)[C@H](CC(C)C)NC1=O)C(C)C]

53. The compound of any one of embodiments 1 to 49, wherein the Extracellular Protein Targeting Ligand targets IgG.

54. The compound of any one of embodiments 1 to 49, wherein the Extracellular Protein Targeting Ligand targets IgE.

55. The compound of any one of embodiments 1 to 49, wherein the Extracellular Protein Targeting Ligand targets IgM.

56. The compound of any one of embodiments 1 to 49, wherein the Extracellular Protein Targeting Ligand targets TNF-α.

57. The compound of any one of embodiments 1 to 49, wherein the Extracellular Protein Targeting Ligand targets IL-1b.

58. The compound of any one of embodiments 1 to 49, wherein the Extracellular Protein Targeting Ligand targets IL-2.

59. The compound of any one of embodiments 1 to 49, wherein the Extracellular Protein Targeting Ligand targets IL-6.

60. The compound of any one of embodiments 1 to 49, wherein the Extracellular Protein Targeting Ligand targets IFN-γ.

61. The compound of any one of embodiments 1 to 49, wherein the Extracellular Protein Targeting Ligand targets VEGF.

62. The compound of any one of embodiments 1 to 49, wherein the Extracellular Protein Targeting Ligand targets TGF-b1.

63. The compound of any one of embodiments 1 to 49, wherein the Extracellular Protein Targeting Ligand targets PCSK-9.

64. A compound selected from:

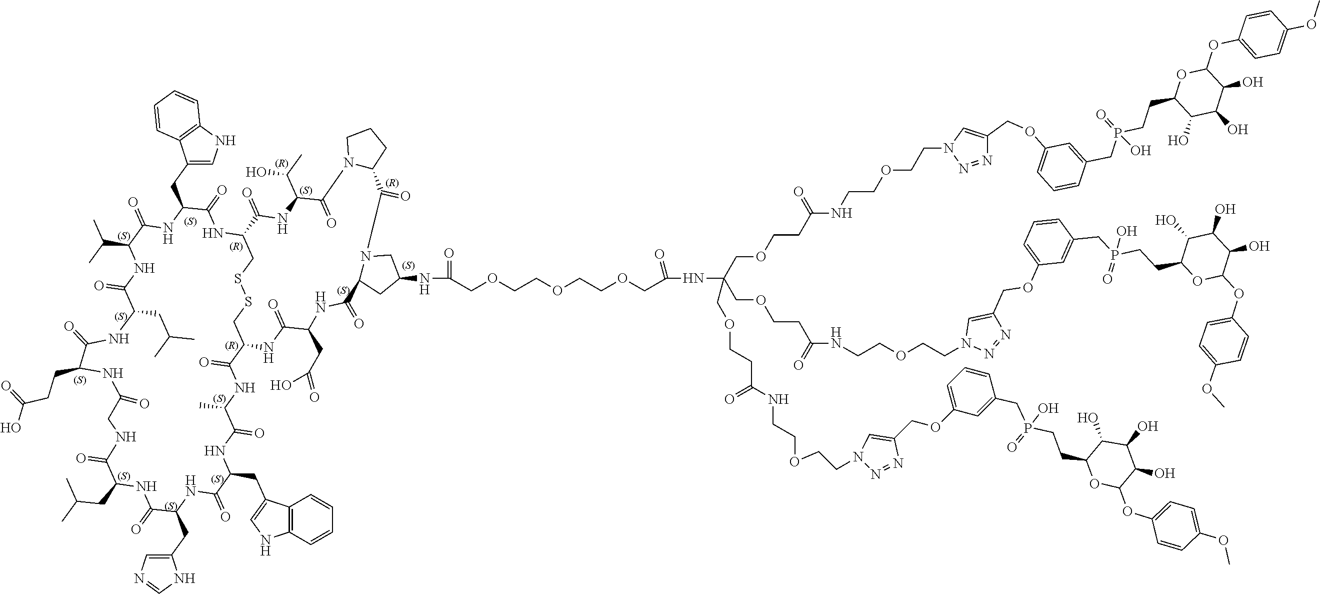

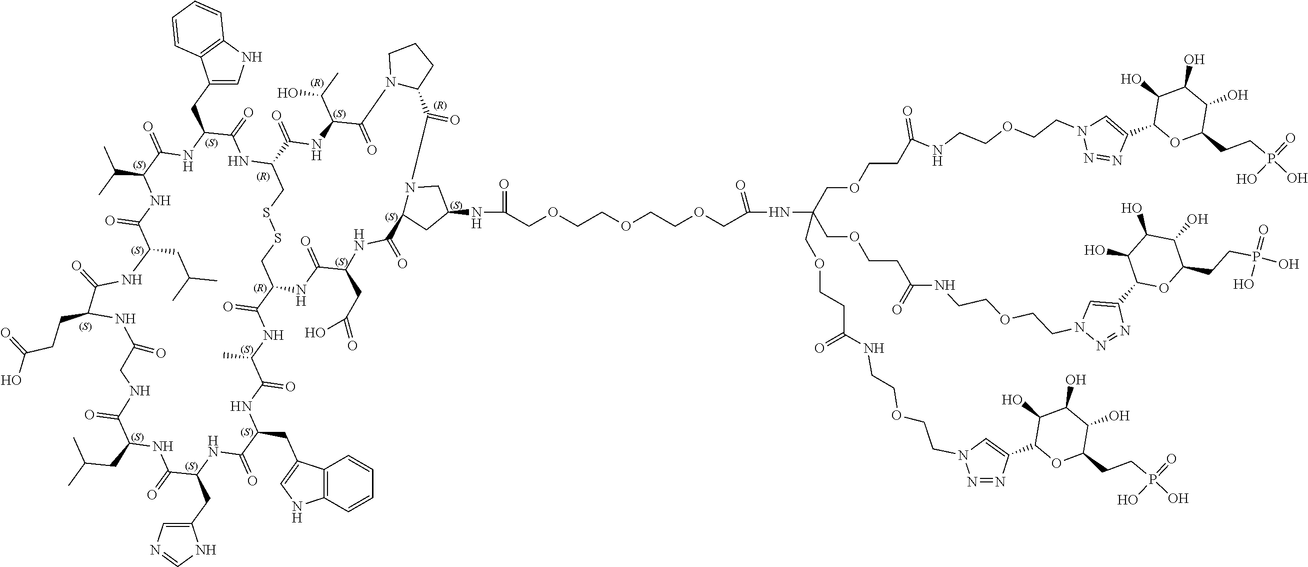

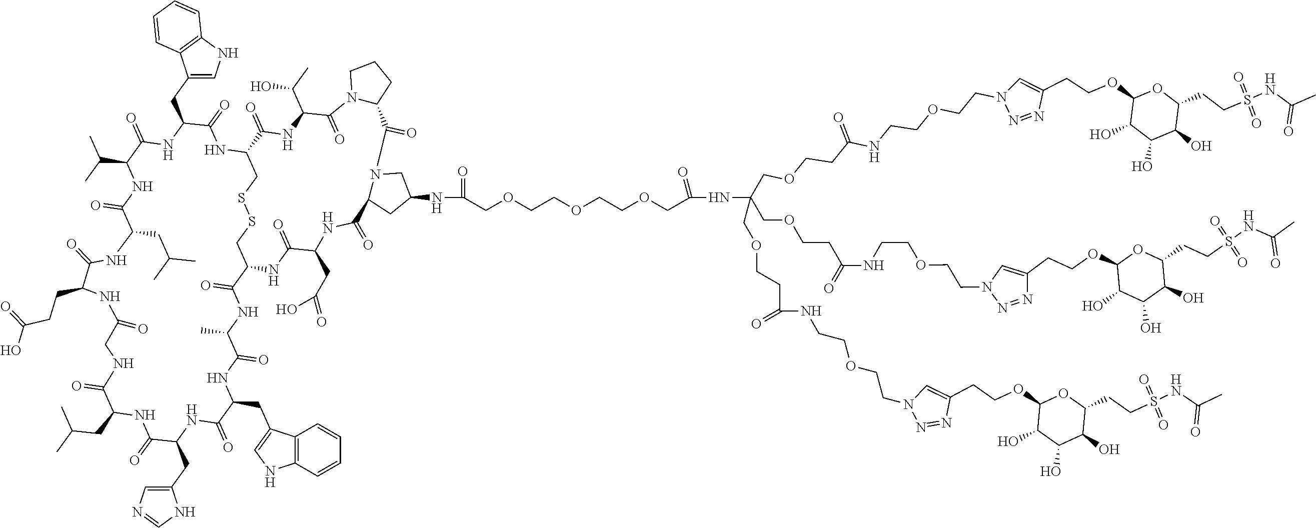

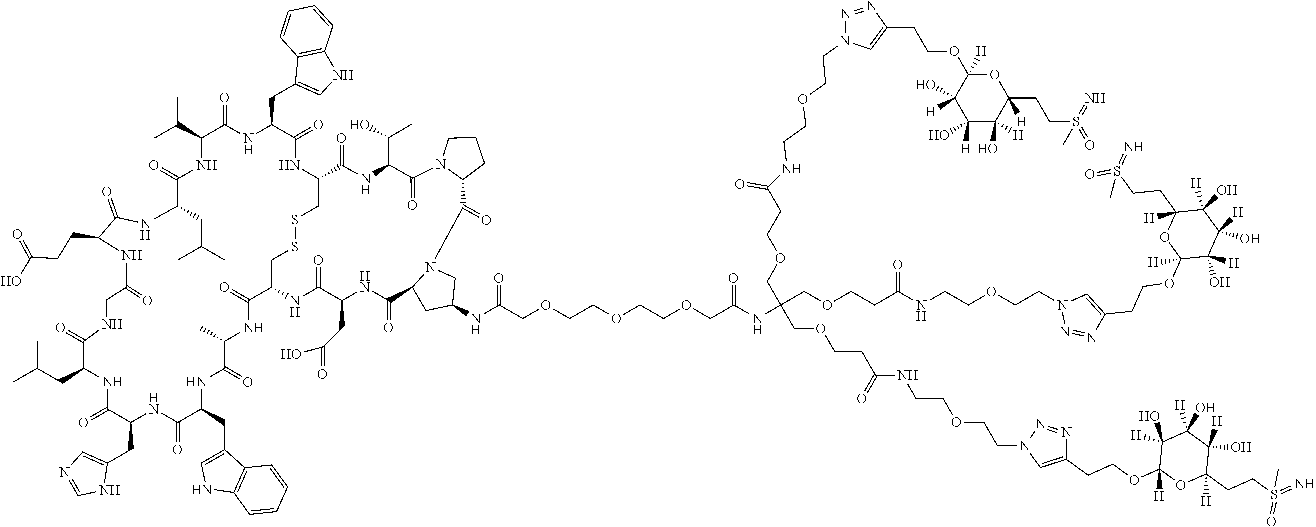

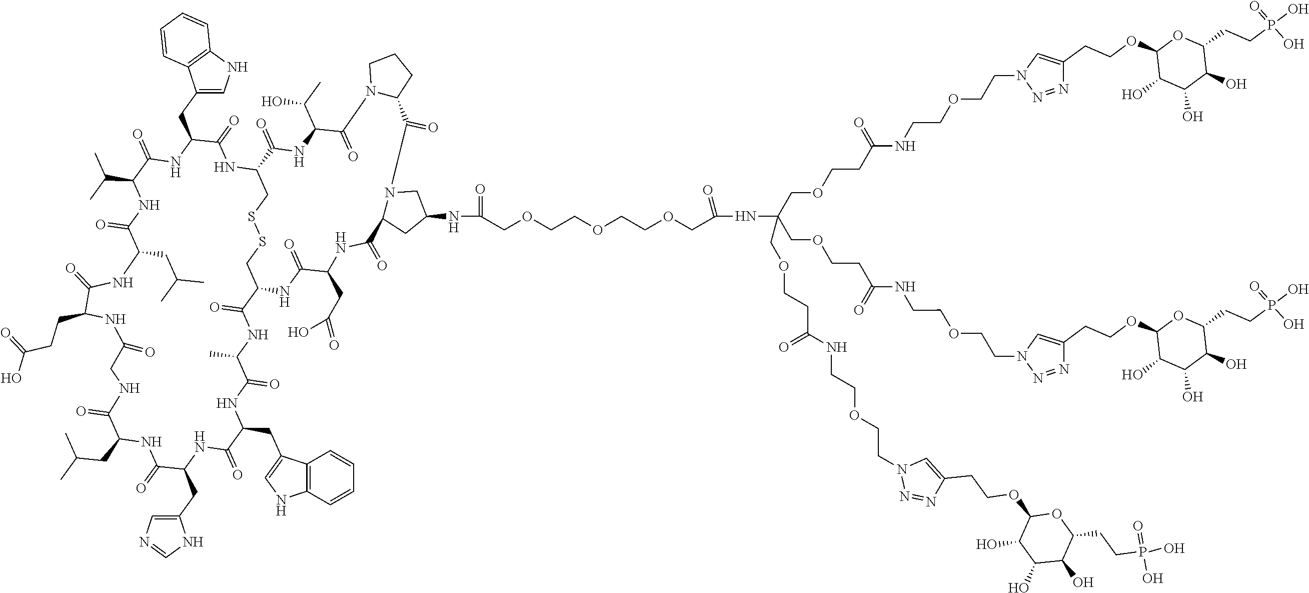

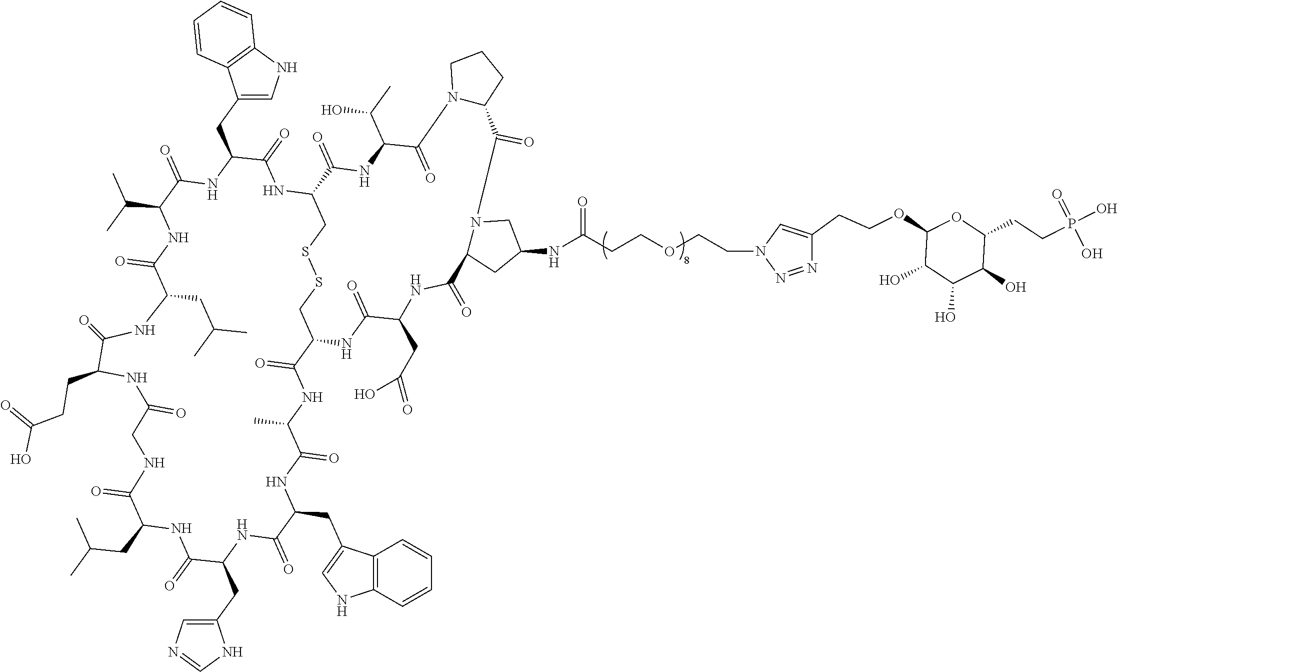

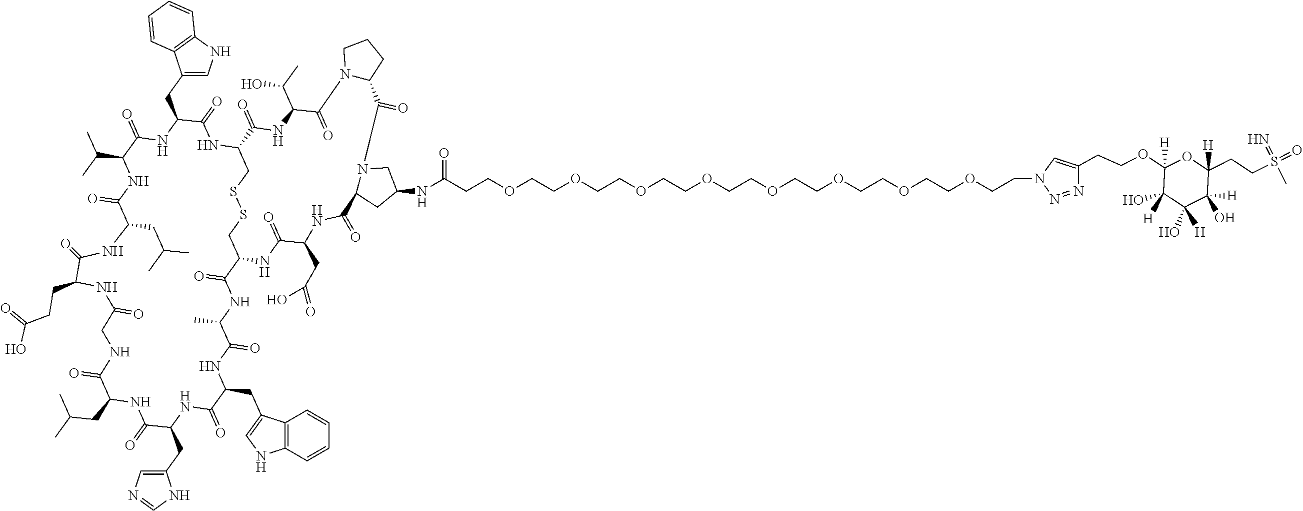 [SMILES: CC(C)C[C@@H]1NC(=O)[C@H](Cc2cnc[nH]2)NC(=O)[C@H](Cc2c[nH]c3ccccc23)NC(=O)[C@H](C)NC(=O)[C@@H]2CSSC[C@H](NC(=O)[C@H](Cc3c[nH]c4ccccc34)NC(=O)[C@H](C(C)C)NC(=O)[C@H](CC(C)C)NC(=O)[C@H](CCC(=O)O)NC(=O)CNC1=O)C(=O)N[C@@H]([C@@H](C)O)C(=O)N1CCC[C@@H]1C(=O)N1C[C@@H](NC(=O)CCOCCOCCOCCOCCOCCOCCOCCOCCn3cc(CCO[C@H]4O[C@H](CCS(C)(=N)=O)[C@@H](O)[C@H](O)[C@@H]4O)nn3)C[C@H]1C(=O)N[C@@H](CC(=O)O)C(=O)N2]

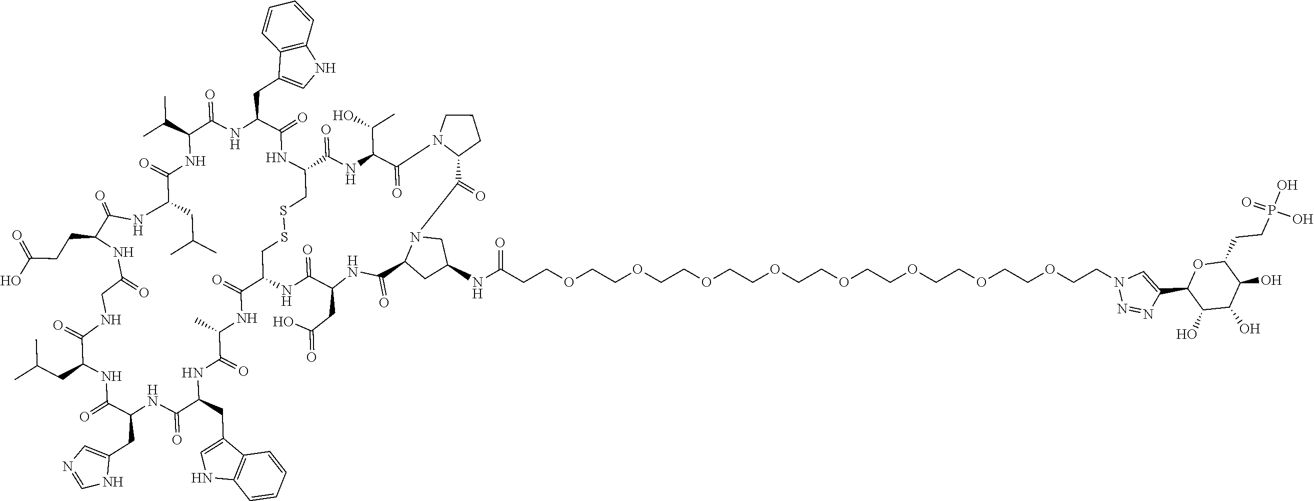 [SMILES: CC(C)C[C@@H]1NC(=O)[C@H](Cc2cnc[nH]2)NC(=O)[C@H](Cc2c[nH]c3ccccc23)NC(=O)[C@H](C)NC(=O)[C@@H]2CSSC[C@H](NC(=O)[C@H](Cc3c[nH]c4ccccc34)NC(=O)[C@H](C(C)C)NC(=O)[C@H](CC(C)C)NC(=O)[C@H](CCC(=O)O)NC(=O)CNC1=O)C(=O)N[C@@H]([C@@H](C)O)C(=O)N1CCC[C@@H]1C(=O)N1C[C@@H](NC(=O)CCOCCOCCOCCOCCOCCOCCOCCOCCn3cc([C@H]4O[C@H](CCP(=O)(O)O)[C@@H](O)[C@H](O)[C@@H]4O)nn3)C[C@H]1C(=O)N[C@@H](CC(=O)O)C(=O)N2]

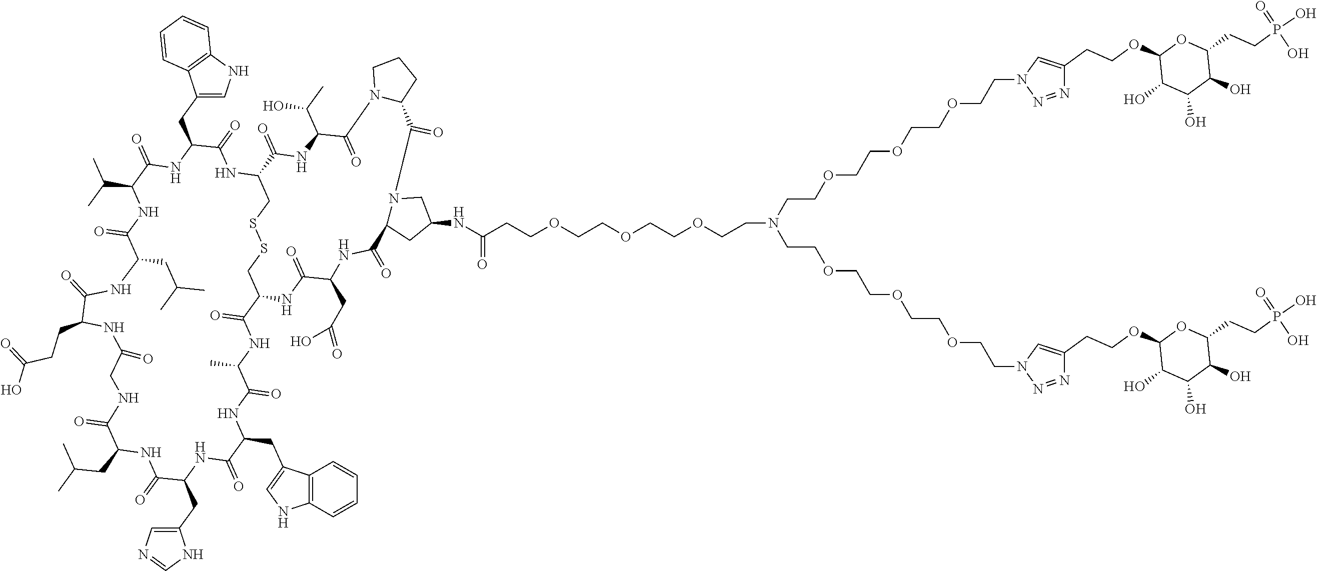 [SMILES: CC(C)C[C@@H]1NC(=O)[C@H](Cc2cnc[nH]2)NC(=O)[C@H](Cc2c[nH]c3ccccc23)NC(=O)[C@H](C)NC(=O)[C@@H]2CSSC[C@H](NC(=O)[C@H](Cc3c[nH]c4ccccc34)NC(=O)[C@H](C(C)C)NC(=O)[C@H](CC(C)C)NC(=O)[C@H](CCC(=O)O)NC(=O)CNC1=O)C(=O)N[C@@H]([C@@H](C)O)C(=O)N1CCC[C@@H]1C(=O)N1C[C@@H](NC(=O)CCOCCOCCOCCN(CCOCCOCCOCCn3cc(CCO[C@H]4O[C@H](CCP(=O)(O)O)[C@@H](O)[C@H](O)[C@@H]4O)nn3)CCOCCOCCOCCn3cc(CCO[C@H]4O[C@H](CCP(=O)(O)O)[C@@H](O)[C@H](O)[C@@H]4O)nn3)C[C@H]1C(=O)N[C@@H](CC(=O)O)C(=O)N2]

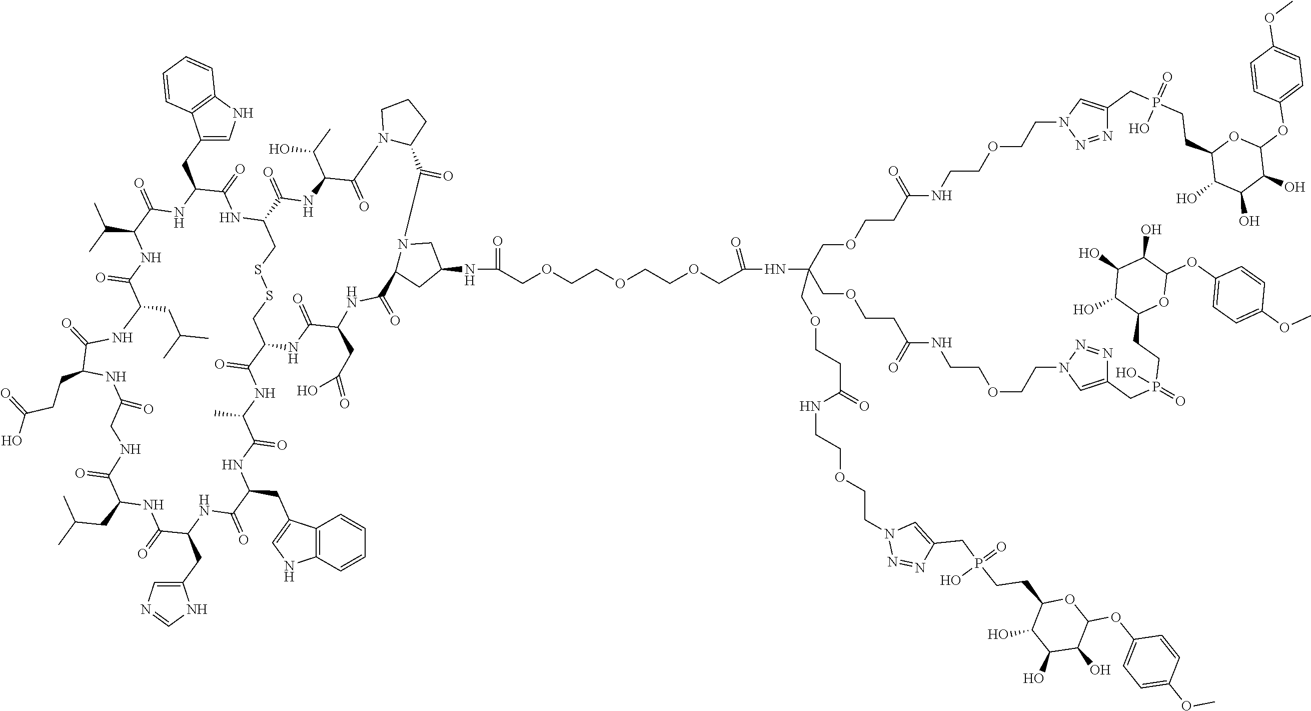

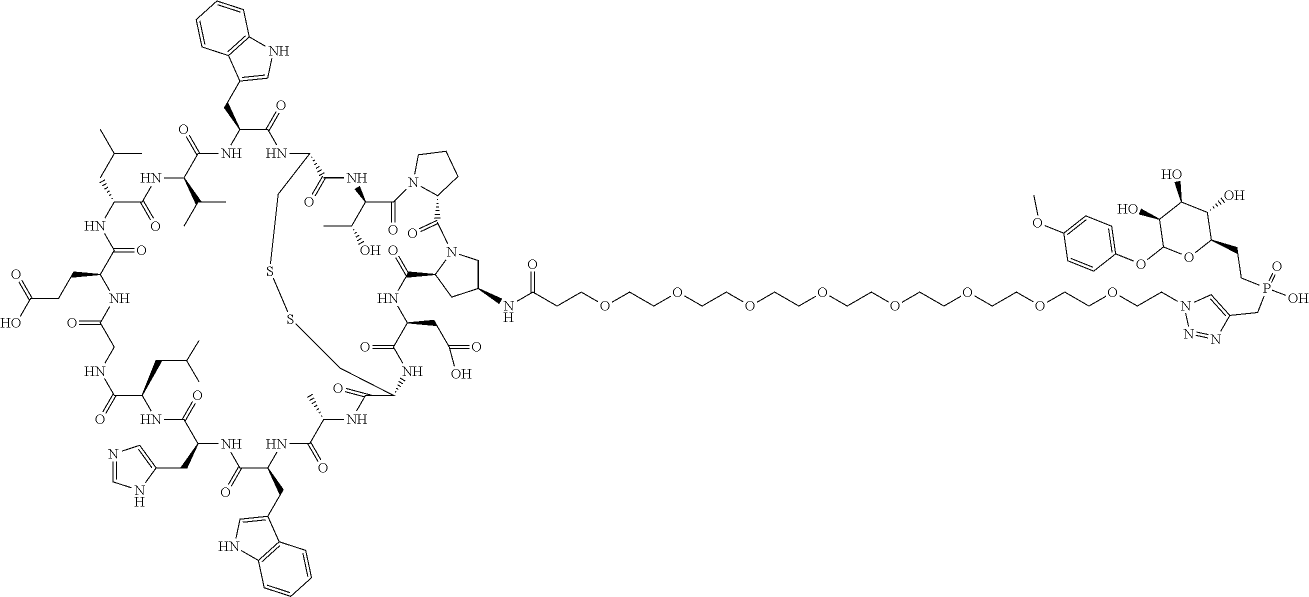 [SMILES: COc1ccc(OC2O[C@H](CCP(=O)(O)Cc3cn(CCOCCOCCOCCOCCOCCOCCOCCOCCC(=O)N[C@H]4C[C@H]5C(=O)N[C@@H](CC(=O)O)C(=O)N[C@H]6CSSC[C@H](NC(=O)[C@H](Cc7c[nH]c8ccccc78)NC(=O)[C@@H](C(C)C)NC(=O)[C@@H](CC(C)C)NC(=O)[C@H](CCC(=O)O)NC(=O)CNC(=O)[C@@H](CC(C)C)NC(=O)[C@H](Cc7cnc[nH]7)NC(=O)[C@H](Cc7c[nH]c8ccccc78)NC(=O)[C@H](C)NC6=O)C(=O)N[C@H]([C@@H](C)O)C(=O)N6CCC[C@@H]6C(=O)N5C4)nn3)[C@@H](O)[C@H](O)[C@@H]2O)cc1]

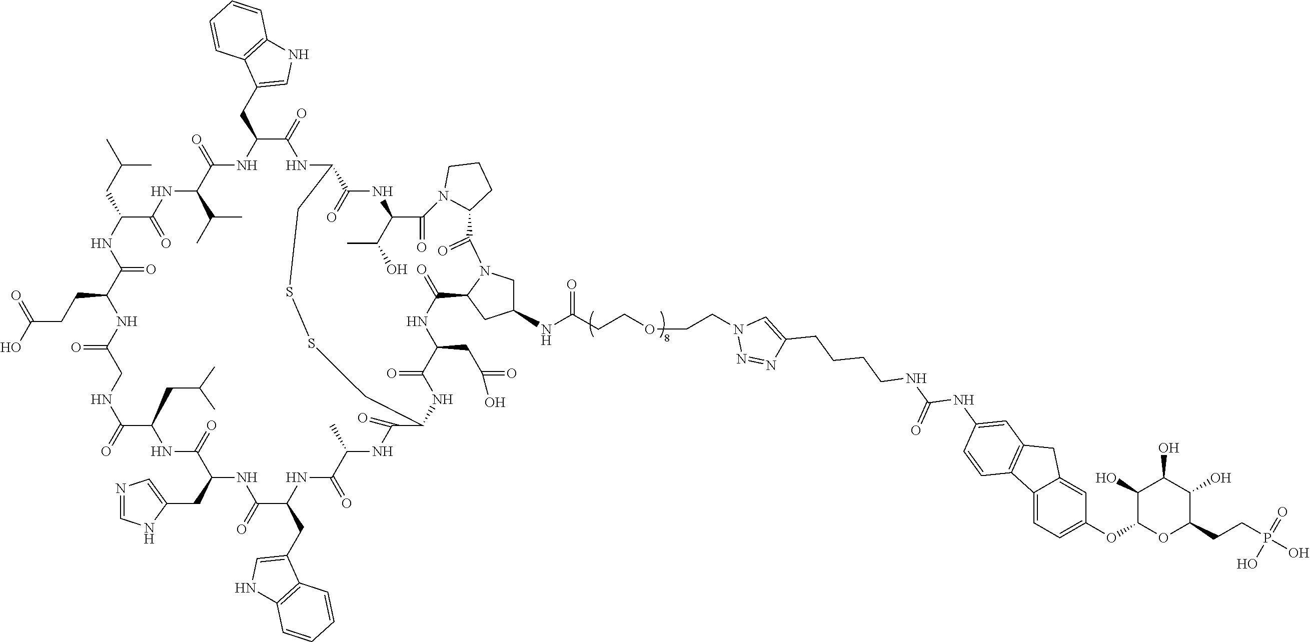

or a pharmaceutically acceptable salt thereof.

65. A pharmaceutical composition comprising a compound of any one of embodiments 1 to 64 and a pharmaceutically acceptable carrier.

66. A method of treating a disorder mediated by an Extracellular Protein comprising administering an effective amount of a compound of any one of embodiments 1 to 64 that includes an Extracellular Protein Targeting Ligand that binds to the Extracellular Protein, or a pharmaceutically acceptable salt thereof, to a patient in need thereof.

67. The method of embodiment 66, wherein the extracellular protein is IgA and the disorder is selected from IgA nephropathy (Berger's disease), celiac disease, Crohn's disease, Henoch-Sconiein purpura (HSP), liner IgA bullous dermatosis, IgA pemphigus, dermatitis herpetiformis, inflammatory bowel disease (IBD), Sjögren's syndrome, ankylosing spondylitis, alcoholic liver cirrhosis, acquired immunodeficiency syndrome, IgA multiple myeloma, α-chain disease, IgA monoclonal gammopathy, monoclonal gammopathy of undetermined significance (MGUS), and linear IgA bullous dermatosis.

68. The method of embodiment 66, wherein the extracellular protein is IgG and the disorder is selected from type 1 autoimmune pancreatitis, interstitial nephritis, Riedel's thyroiditis, storiform fibrosis, Mikulicz's disease, Küttner's tumor, inflammatory pseudotumors, mediastinal fibrosis, retroperitoneal fibrosis (Ormond's disease), aortitis, periaortitis, proximal biliary strictures, idiopathic hypocomplementic tubulointerstitial nephritis, multifocal fibrosclerosis, pachymeningitis, pancreatic enlargement, tumefactive lesions, pericarditis, rheumatoid arthritis (RA), inflammatory bowel disease, multiple sclerosis, myasthenic gravis, thyroid eye disease, chronic inflammatory demyelinating polyneuropathy, warm autoimmune hemolytic anemia, ankylosing spondylitis, primary Sjögren's syndrome, psoriatic arthritis, and systemic lupus erythematosus (SLE), sclerosing cholangitis, and IgG monoclonal gammopathy, monoclonal gammopathy of undetermined significance (MGUS).

69. The method of embodiment 66, wherein the extracellular protein is IgE and the disorder is selected from atopic asthma, allergic rhinitis, atopic dermatitis, IgE-mediated food allergy, IgE-mediated animal allergies, allergic conjunctivitis, allergic urticaria, anaphylactic shock, nasal polyposis, keratoconjunctivitis, mastocytosis, and eosinophilic gastrointestinal disease, bullous pemphigoid, chemotherapy induced hypersensitivity reaction, seasonal allergic rhinitis, interstitial cystitis, eosinophilic esophagitis, angioedema, acute interstitial nephritis, atopic eczema, eosinophilic bronchitis, chronic obstructive pulmonary disease, gastroenteritis, hyper-IgE syndrome (Job's Syndrome), IgE monoclonal gammopathy, and monoclonal gammopathy of undetermined significance (MGUS).

70. The method of embodiment 66, wherein the disorder is dementia or Alzheimer's disease.

71. The method of embodiment 66, wherein the extracellular protein is TNF-α and the disorder is selected from rheumatoid arthritis, inflammatory bowel disease, graft-vs-host disease, ankylosing spondylitis, psoriasis, hidradenitis suppurativa, refractory asthma, systemic lupis erthyematosus, diabetes, and the induction of cachexia.

72. The method of embodiment 66, wherein the extracellular protein is IL-2 and the disorder is selected from host versus graft rejection in transplants and autoimmune disorders.

73. The method of embodiment 66, wherein the extracellular protein is IL-6 and the disorder is selected from Castleman's disease, metastatic castration-associated prostate cancer, renal cell carcinoma, large-cell lung carcinoma, ovarian cancer, rheumatoid arthritis, and asthma.

74. The method of embodiment 66, wherein the extracellular protein is IFN-γ and the disorder is selected from rheumatoid arthritis, multiple sclerosis (MS), corneal transplant rejection, and various autoimmune skin diseases such as psoriasis, alopecia areata, vitiligo, and acne vulgaris.

75. The method of embodiment 66, wherein the disorder is a cancer.

76. A compound of Formula IV:

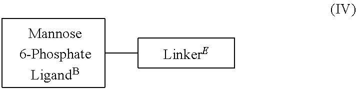

or a salt thereof;
wherein:
Mannose 6-Phosphate Ligand^{B} is selected from:

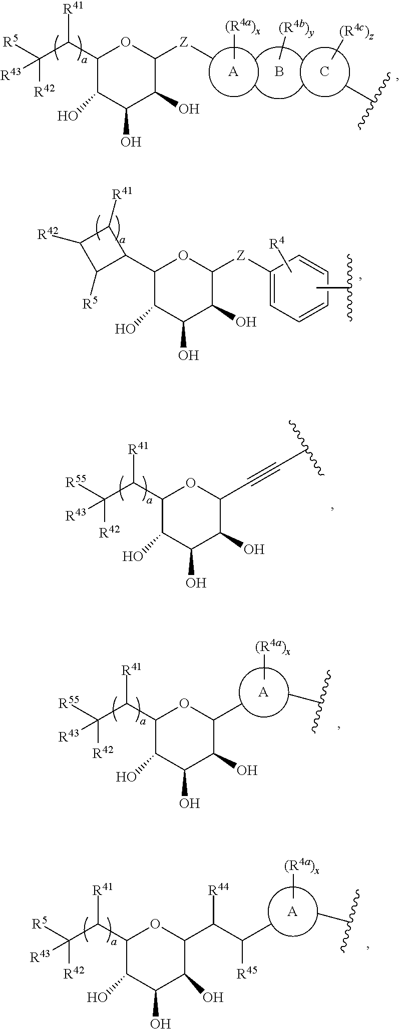

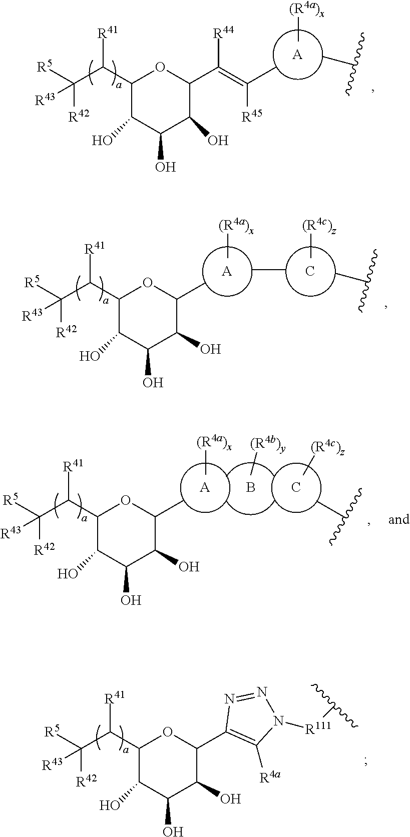

or a salt thereof;
Linker^{E} is selected from

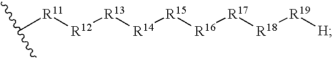

R¹¹, R¹², R¹³, R¹⁴, R¹⁵, R¹⁶, R¹⁷, R¹⁸, and R¹⁹ are independently at each occurrence selected from the group consisting of a bond, alkyl, —C(O)—, —C(O)O—, —OC(O)—, —SO₂—, —S(O)—, —C(S)—, —C(O)NR⁶—, —NR⁶C(O)—, —C(O)N(OH)—, —N(OH)C(O)—, —O—, —S—, —NR⁶—, —C(R²¹R²¹)—, —S(O)(═N—R⁴)—, —S(O)(═NR), —P(O)(R³)O—, —P(O)(R³)—, a divalent residue of a natural or unnatural amino acid, alkenyl, alkynyl, haloalkyl, alkoxy, aryl, heterocycle, heteroaryl, —CH₂CH₂—[O—(CH₂)₂]ₙ—O—, —CH₂CH₂—[O—(CH₂)₂]ₙ—NR⁶—, —CH₂CH₂—[O—(CH₂)₂]ₙ—, -[—(CH₂)₂—O-]ₙ-, —[O—(CH₂)₂]ₙ—, —[O—CH(CH₃)C(O)]ₙ—, —[C(O)—CH(CH₃)—O]ₙ—, —[O—CH₂C(O)]ₙ—, —[C(O)—CH₂—O]ₙ—, a divalent residue of a fatty acid, a divalent residue of an unsaturated or saturated mono- or di-carboxylic acid; each of which is optionally substituted with 1, 2, 3, or 4 substituents independently selected from R²¹;
n is independently selected at each instance from 0, 1, 2, 3, 4, 5, 6, 7, 8, 9, or 10;
R²¹ is independently at each occurrence selected from the group consisting of hydrogen, alkyl, alkenyl, alkynyl, F, Cl, Br, I, hydroxyl, alkoxy, azide, amino, cyano, —NR⁶R⁷, —NR⁸SO₂R³, —NR⁸S(O)R³, haloalkyl, aryl, heteroaryl, and heterocycle;
R¹¹¹ is aryl, heteroaryl, heterocycle, C₃-C₁₀alkyl, alkenyl, or alkynyl each of which is optionally independently substituted by 1, 2; or 3 R⁴⁶ substituents;
a, x, y, and z are independently 0, 1, 2, or 3;
Z is NR⁸, O, S, NC(O)R³, or CR⁴R⁸,
\textcircled{A} is cycloalkyl, heterocycle, aryl or heteroaryl;
\textcircled{B} is a nonaromatic cycloalkyl or heterocycle;
\textcircled{C} is aryl or heteroaryl;
R³ at each occurrence is independently selected from hydrogen, alkyl, haloalkyl, arylalkyl, heteroarylalkyl, alkenyl, alkynyl, aryl, heteroaryl, heterocycle, —OR⁸, and —NR⁸R⁹;
R⁴, R^{4a}, R^{4b}, and R^{4c} are independently selected at each occurrence from hydrogen, F, Cl, Br, I, alkyl, haloalkyl, arylalkyl, heteroarylalkyl, alkenyl, alkynyl, aryl, heteroaryl, heterocycle, cyano, —OR⁶, —NR⁶R⁷, C(O)R³, S(O)R³, C(S)R³, and S(O)₂R³;
R⁴¹, R⁴², R⁴³, R⁴⁴, and R⁴⁵ are independently selected at each occurrence from hydrogen, alkyl, haloalkyl, F, arylalkyl, heteroarylalkyl, alkenyl, alkynyl, aryl, heteroaryl, and heterocycle;
R⁵⁰ is hydrogen, alkyl, haloalkyl, arylalkyl, heteroarylalkyl, alkenyl, alkynyl, aryl, heteroaryl, heterocycle, —OR⁶, or—NR⁶R⁷;
R⁵ is selected from

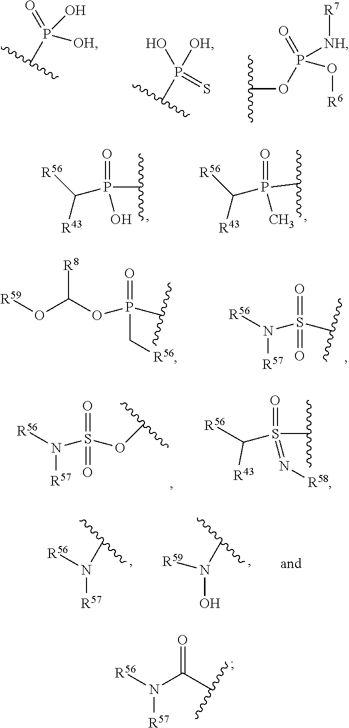

R⁵⁵ is selected from

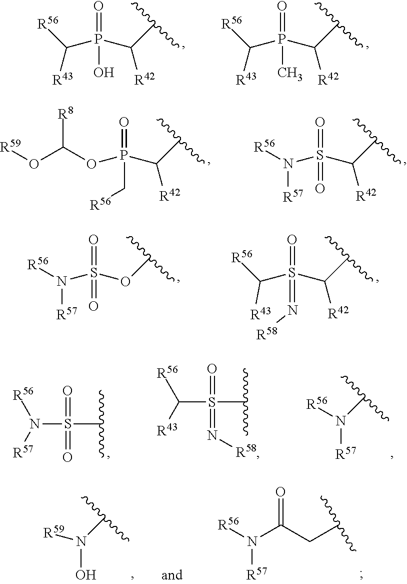

R⁵⁶ is independently selected from hydrogen, alkyl, alkenyl, alkynyl, haloalkyl, —O-alkyl, hydroxyl, cyano, heterocyclyl, aryl, and heteroaryl wherein each group except for hydrogen may optionally be substituted with 1, 2, or 3 independently selected R^{9a} substituents as allowed by valence;
R⁵⁷ is selected from hydrogen, alkyl, alkenyl, alkynyl, haloalkyl, —O-alkyl, aryl, heteroaryl, C(O) H, C(O) alkyl, C(O) alkenyl, C(O) alkynyl, C(O) haloalkyl, C(O) aryl, C(O)heteroaryl, C(O)O-alkyl, C(O)O-alkenyl, C(O)O-alkynyl, C(O)O-haloalkyl, C(O)O-aryl, C(O)O-heteroaryl and

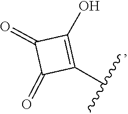 [SMILES: *c1c(O)c(=O)c1=O]

wherein each group except for hydrogen may optionally be substituted with 1, 2, or 3 independently selected Rob substituents as allowed by valence;
or R⁵⁶, R⁵⁷, and the nitrogen to which they are attached form a 5 to 7 membered heterocyclic ring which can be optionally substituted by alkyl, heterocycle, aryl, and heteroaryl;
R⁵⁸ is selected from hydrogen, alkyl, C(O) alkyl, C(O)O-alkyl, C(O) aryl, C(O)heteroaryl, C(O)O-aryl, C(O)O-arylalkyl, and C(O)O-heteroaryl, wherein each group may optionally be substituted with 1, 2, or 3 independently selected R^{9c} substituents as allowed by valence;
R⁵⁹ is selected from C(O) alkyl, C(O)O-alkyl, C(O) aryl, C(O)heteroaryl, C(O)O-aryl, and C(O)O-heteroaryl, wherein each group may optionally be substituted with 1, 2, or 3 independently selected R^{9d} substituents as allowed by valence;
R⁶ and R⁷ are independently selected at each occurrence from hydrogen, alkyl, arylalkyl, heteroarylalkyl, alkenyl, alkynyl, aryl, haloalkyl, heteroaryl, heterocycle, -alkyl—OR⁸, -alkyl—NR⁸R⁹, C(O)R³, S(O)R³, C(S)R³, and S(O)₂R³;
R⁸ is independently selected at each occurrence from hydrogen, alkyl, arylalkyl, heteroarylalkyl, alkenyl, alkynyl, aryl, heteroaryl, and heterocycle;
R⁹ is independently selected at each occurrence from hydrogen, alkyl, arylalkyl, heteroarylalkyl, alkenyl, alkynyl, aryl, heteroaryl, and heterocycle;
R^{9a}, R^{9b}, R^{9c}, and R^{9d} are independently selected at each occurrence from hydrogen, F, Cl, Br, I, alkyl, haloalkyl, arylalkyl, heteroarylalkyl, alkenyl, alkynyl, aryl, heteroaryl, heterocycle, cyano, —OR⁶, —NR⁶R⁷, C(O)R³, S(O)R³, C(S)R³, and S(O)₂R³.

77. The compound of embodiment 76, wherein R⁵ is selected from:

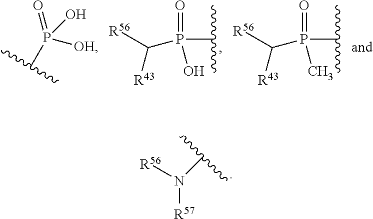

78. The compound of embodiment 76, wherein R⁵⁵ is selected from:

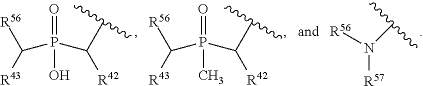

79. The compound of any one of embodiments 76-78, wherein R⁴¹ is hydrogen.

80. The compound of any one of embodiments 76-78, wherein R⁴² is hydrogen.

81. The compound of embodiment 76 of Formula:

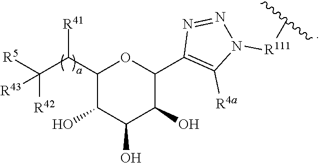

82. The compound of embodiment 81, wherein R¹¹¹ is cycloalkyl, aryl, heteroaryl, C₃-Cioalkyl, heterocycle, or alkynyl.

83. The compound of embodiment 81, wherein R¹¹¹ is aryl.

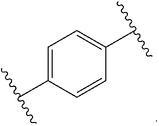 [SMILES: *c1ccc(*)cc1]

84. The compound of embodiment 81, wherein R¹¹¹ is

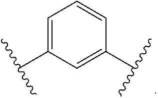 [SMILES: *c1cccc(*)c1]

85. The compound of embodiment 81, wherein R¹¹¹ is

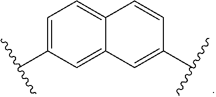 [SMILES: *c1ccc2ccc(*)cc2c1]

86. The compound of embodiment 81, wherein R¹¹¹ is

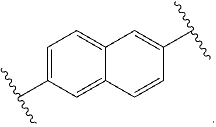 [SMILES: *c1ccc2cc(*)ccc2c1]

87. The compound of embodiment 81, wherein R¹¹¹ is

88. The compound of embodiment 81, wherein R¹¹¹ is heteroaryl.

89. The compound of embodiment 81, wherein R¹¹¹ is

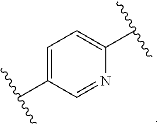 [SMILES: *c1ccc(*)nc1]

90. The compound of embodiment 81, wherein R¹¹¹ is

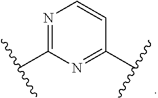 [SMILES: *c1ccnc(*)n1]

91. The compound of embodiment 81, wherein R¹¹¹ is

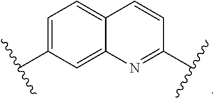 [SMILES: *c1ccc2ccc(*)nc2c1]

92. The compound of embodiment 81, wherein R¹¹¹ is

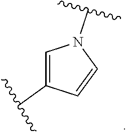 [SMILES: *c1ccn(*)c1]

93. The compound of embodiment 81, wherein R¹¹¹ is

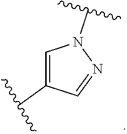 [SMILES: *c1cnn(*)c1]

94. The compound of embodiment 81, wherein R¹¹¹ is cycloalkyl or C₃-C₁₀alkyl.

95. The compound of embodiment 81, wherein R¹¹¹ is

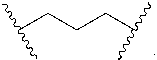 [SMILES: *CCC*]

96. The compound of embodiment 81, wherein R¹¹¹ is

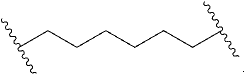 [SMILES: *CCCCCC*]

97. The compound of embodiment 81, wherein R¹¹¹ is

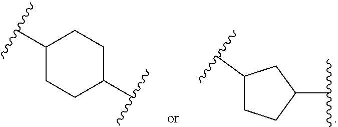 [SMILES: *C1CCC(*)CC1 ; *C1CCC(*)C1]

98. The compound of embodiment 81, wherein R¹¹¹ is heterocycle.

99. The compound of embodiment 81, wherein R¹¹¹ is

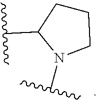 [SMILES: *C1CCCN1*]

100. The compound of embodiment 81, wherein R¹¹¹ is

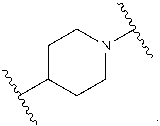 [SMILES: *C1CCN(*)CC1]

101. The compound of embodiment 81, wherein R¹¹¹ is alkynyl.

102. The compound of embodiment 81, wherein R¹¹¹ is

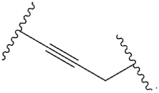 [SMILES: *C#CC*]

In alternative embodiments the Mannose 6-Phosphate Ligand is of Formula:

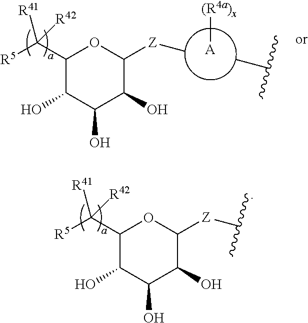

In certain embodiments, the Mannose 6-Phosphate Ligand is

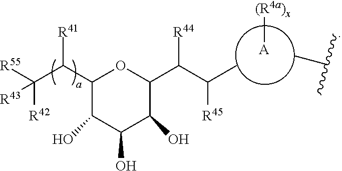

In certain embodiments, the Mannose 6-Phosphate Ligand is selected from

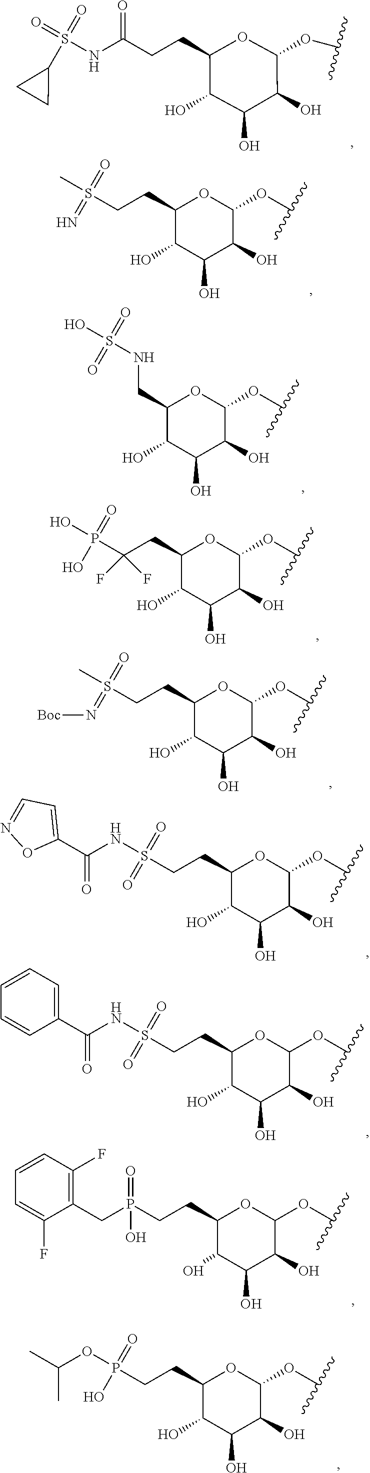

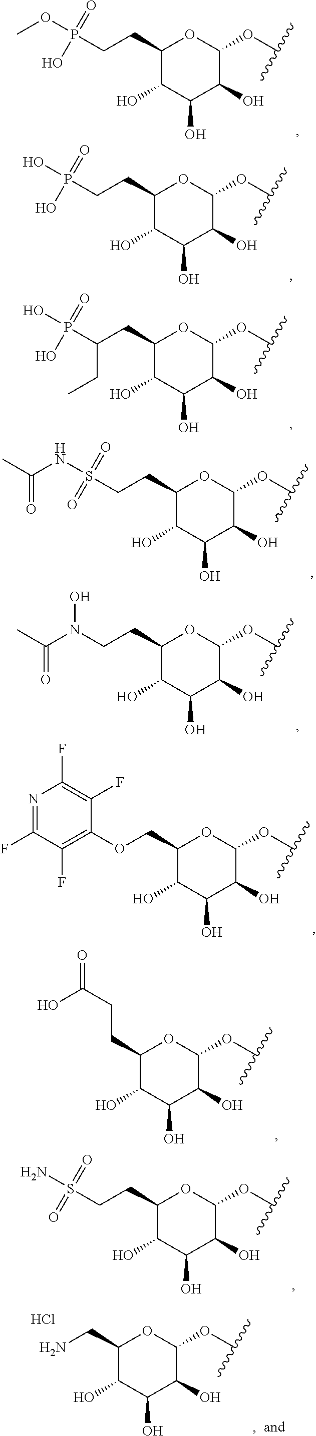

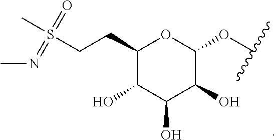 [SMILES: *O[C@H]1O[C@H](CCS(C)(=O)=NC)[C@@H](O)[C@H](O)[C@@H]1O]

In certain embodiments, the Mannose 6-Phosphate Ligand is selected from

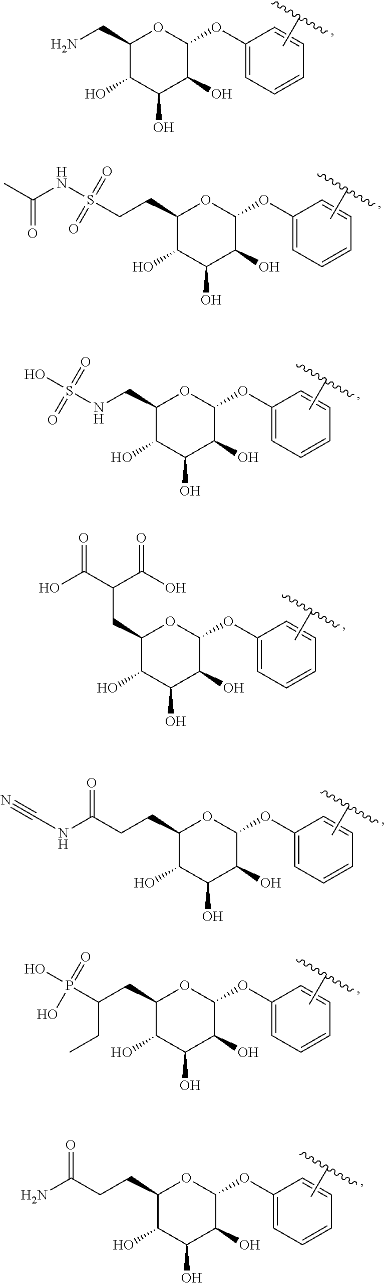

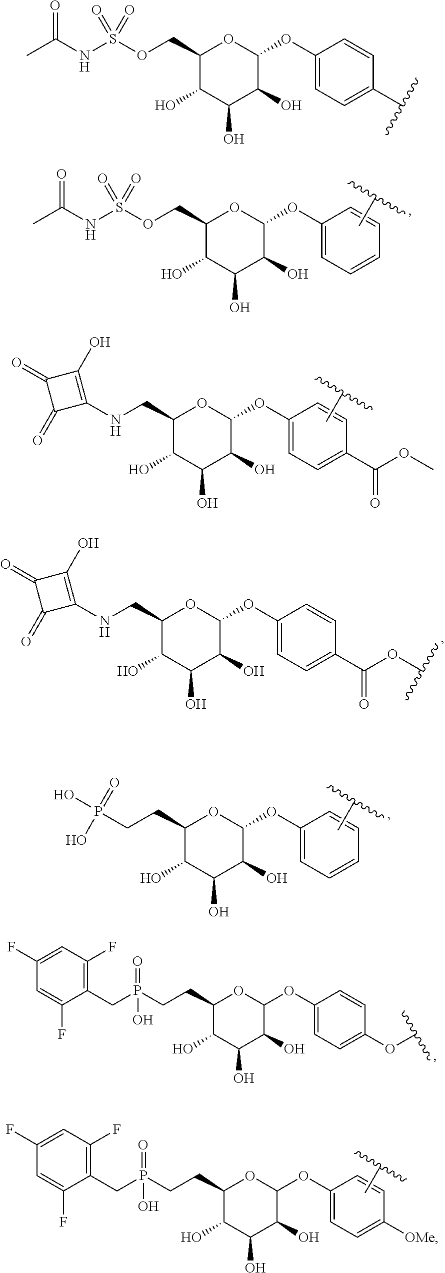

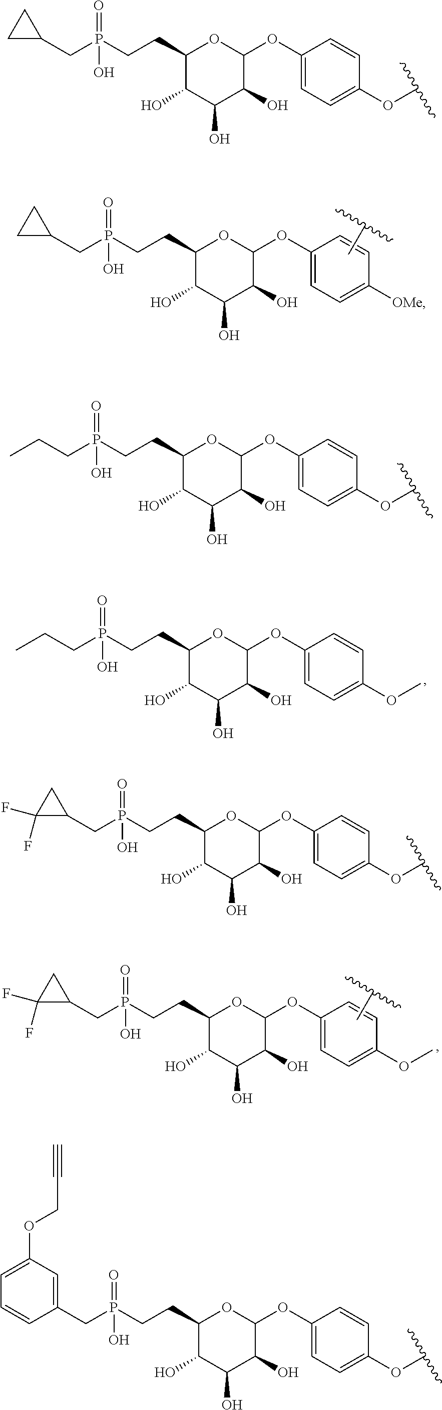

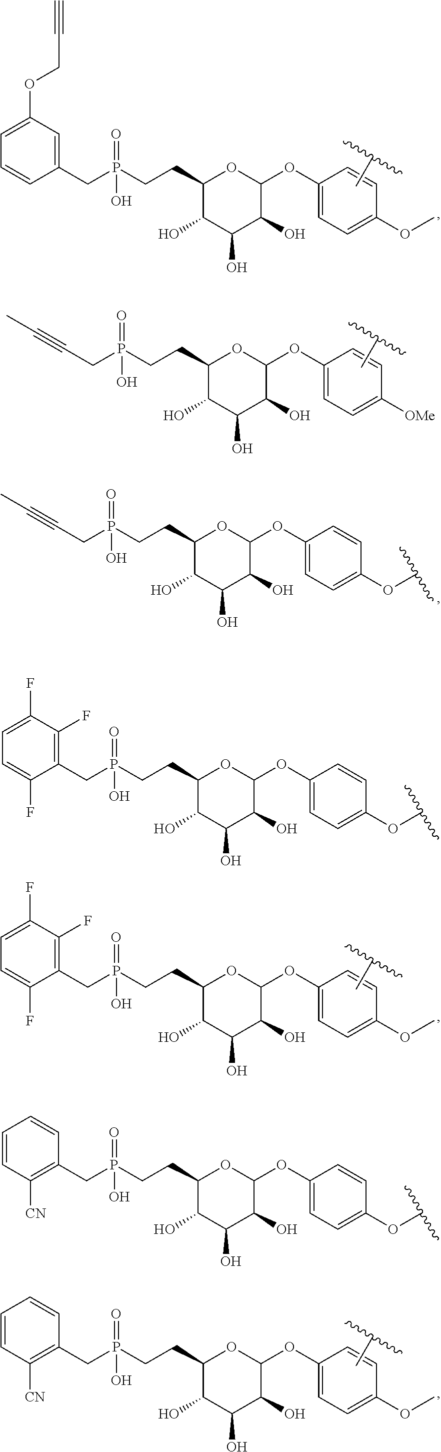

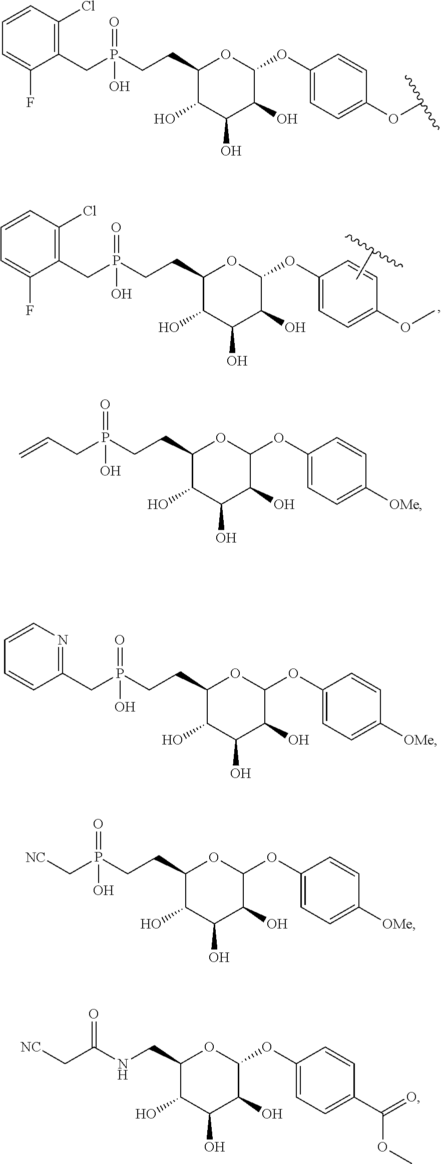

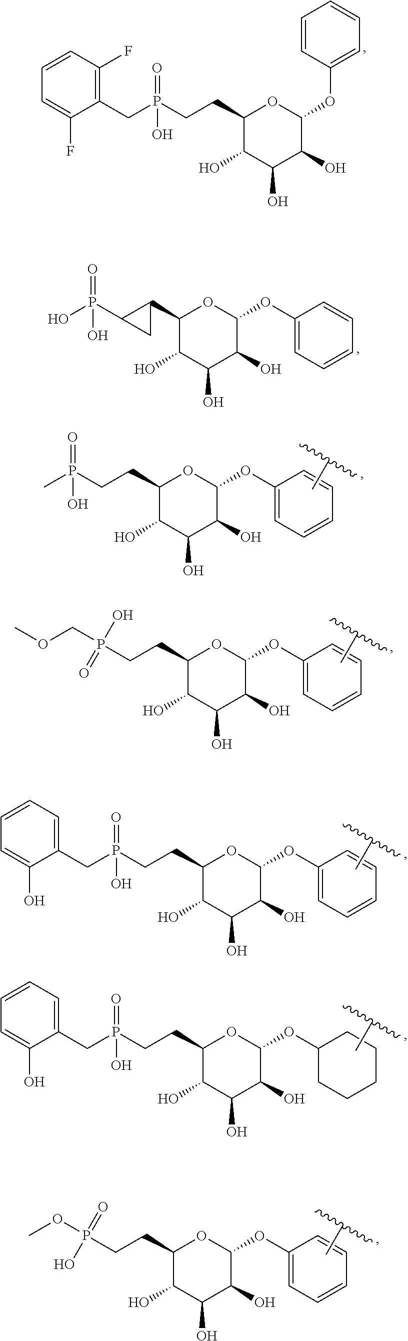

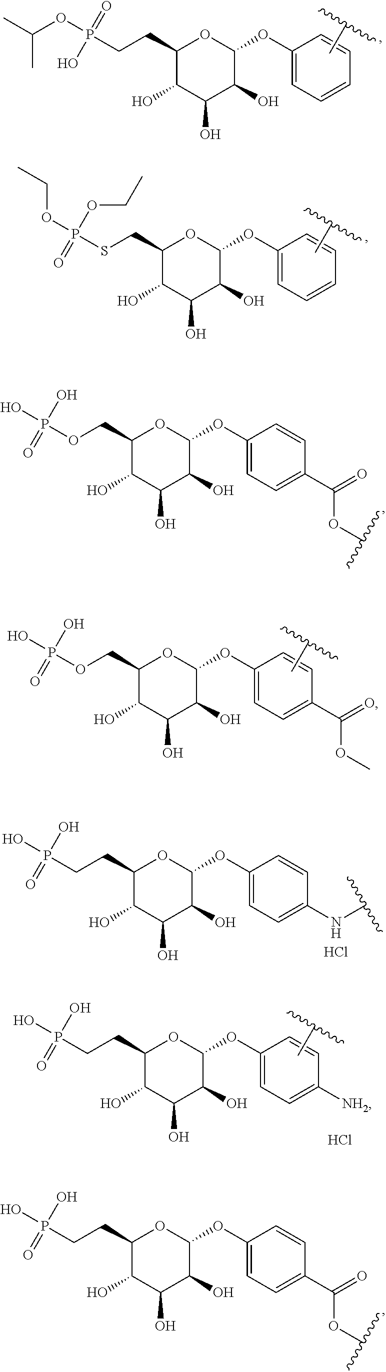

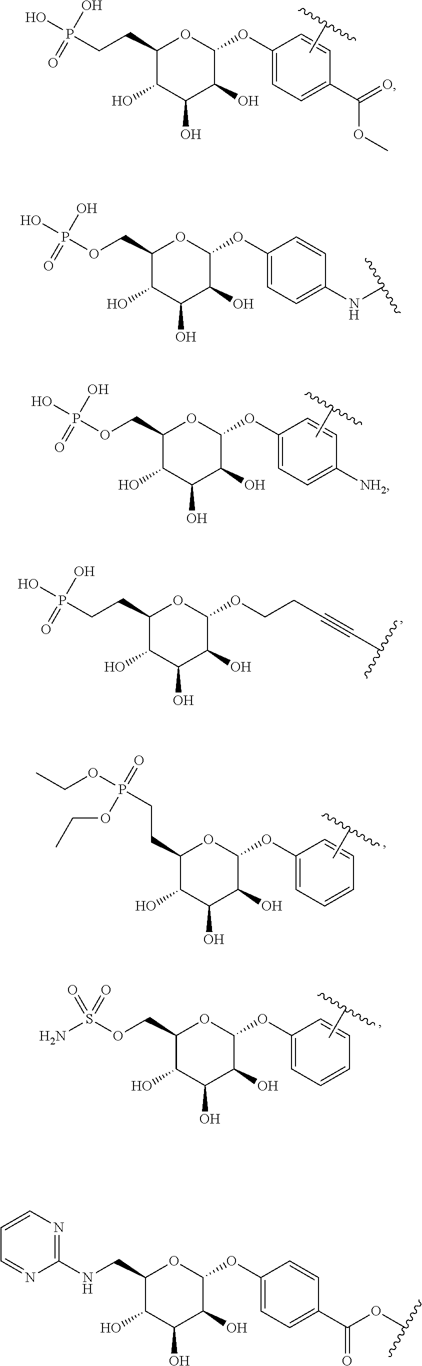

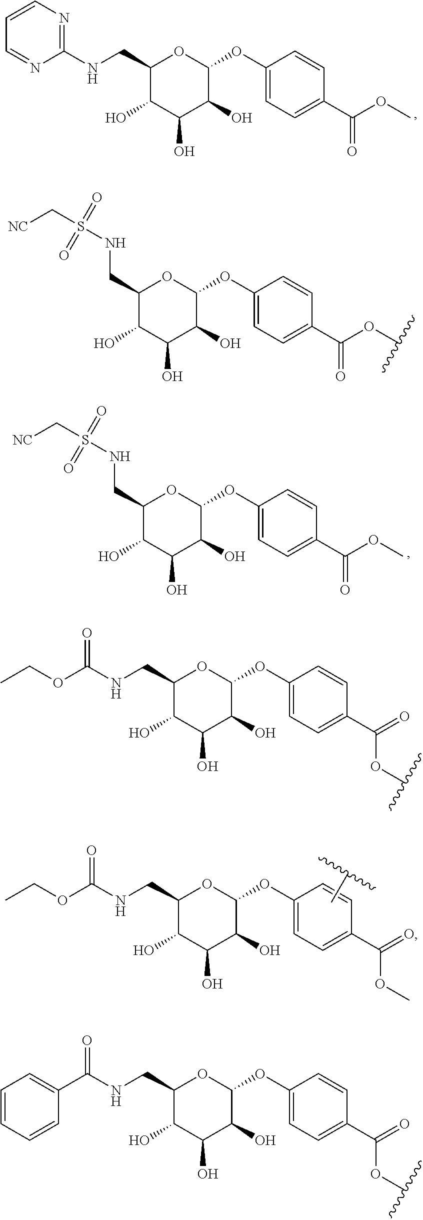

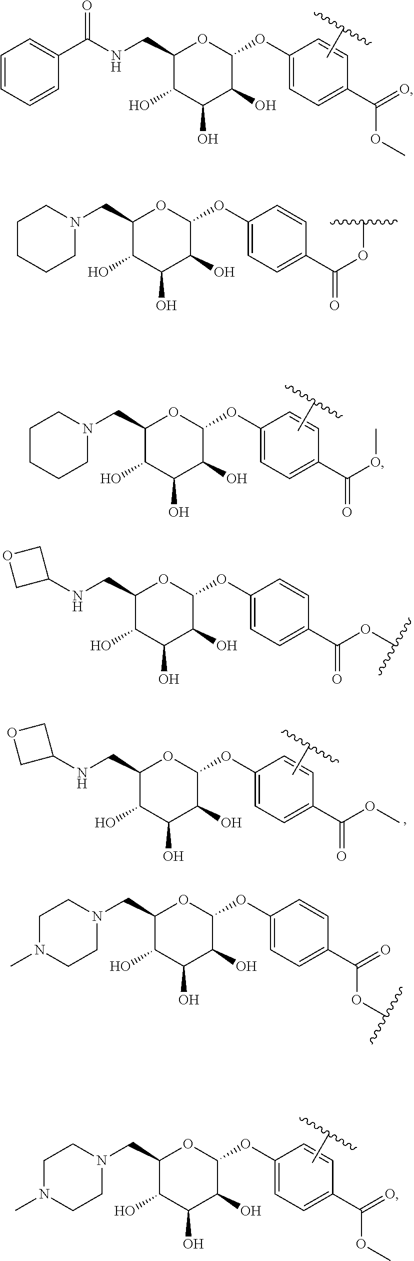

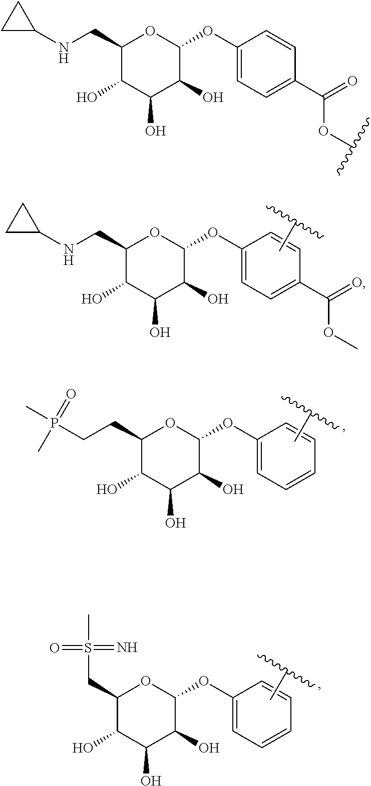

In certain embodiments, the Mannose 6-Phosphate Ligand is selected from

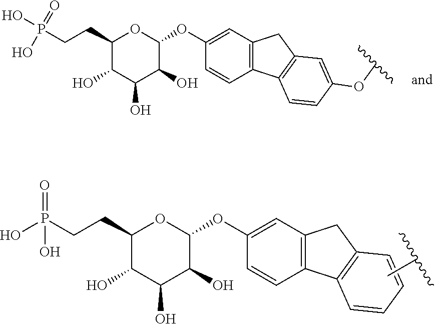

In certain embodiments, the Mannose 6-Phosphate Ligand is selected from

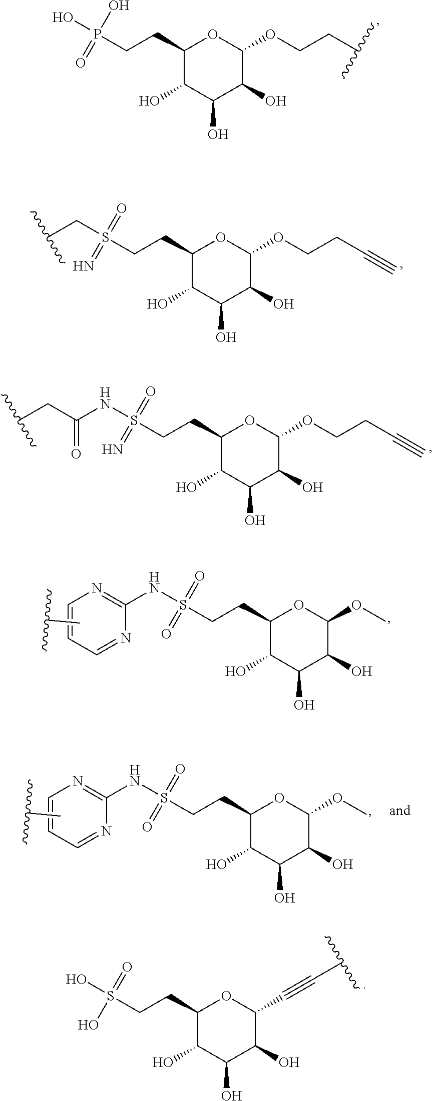

In certain embodiments, the Mannose 6-Phosphate Ligand is selected from

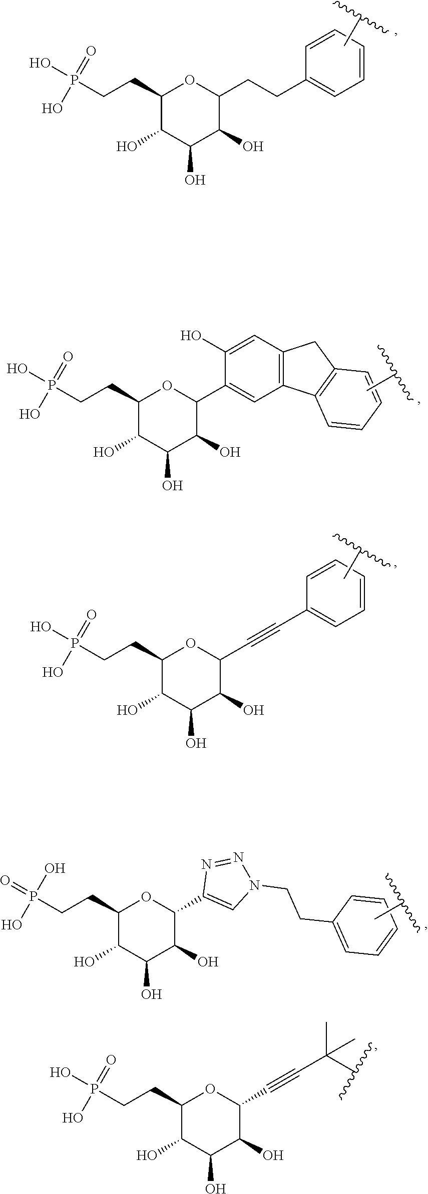

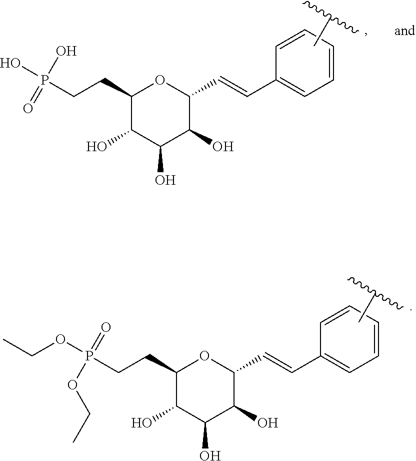

In certain embodiments, the Mannose 6-Phosphate Ligand is

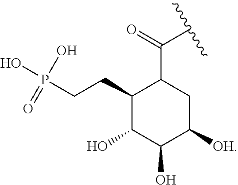 [SMILES: *C(=O)C1C[C@@H](O)[C@@H](O)[C@H](O)[C@H]1CCP(=O)(O)O]

In certain embodiments, the Mannose 6-Phosphate Ligand is selected from

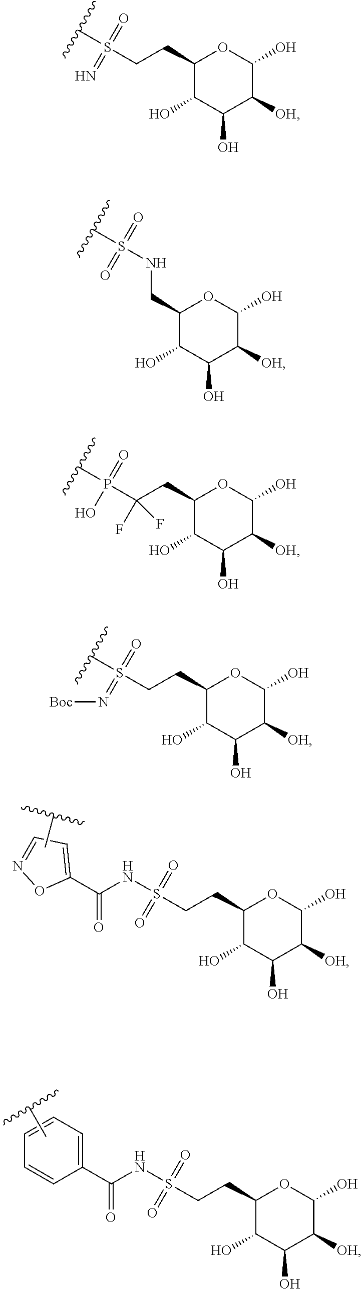

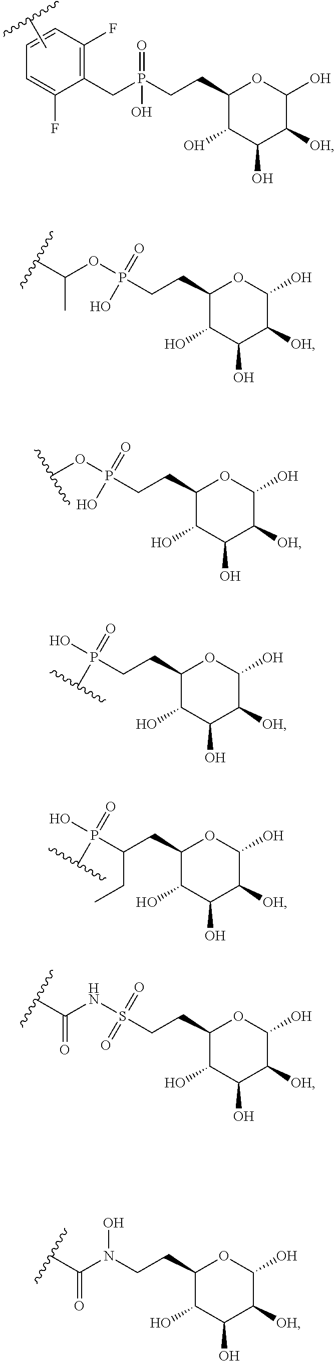

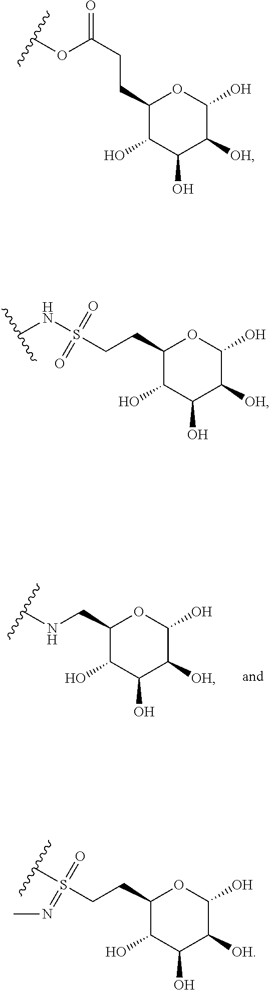 [SMILES: *OC(=O)CC[C@H]1O[C@H](O)[C@@H](O)[C@@H](O)[C@@H]1O ; *NS(=O)(=O)CC[C@H]1O[C@H](O)[C@@H](O)[C@@H](O)[C@@H]1O ; *NC[C@H]1O[C@H](O)[C@@H](O)[C@@H](O)[C@@H]1O ; *S(=O)(CC[C@H]1O[C@H](O)[C@@H](O)[C@@H](O)[C@@H]1O)=NC]

In certain embodiments, the Mannose 6-Phosphate Ligand is selected from

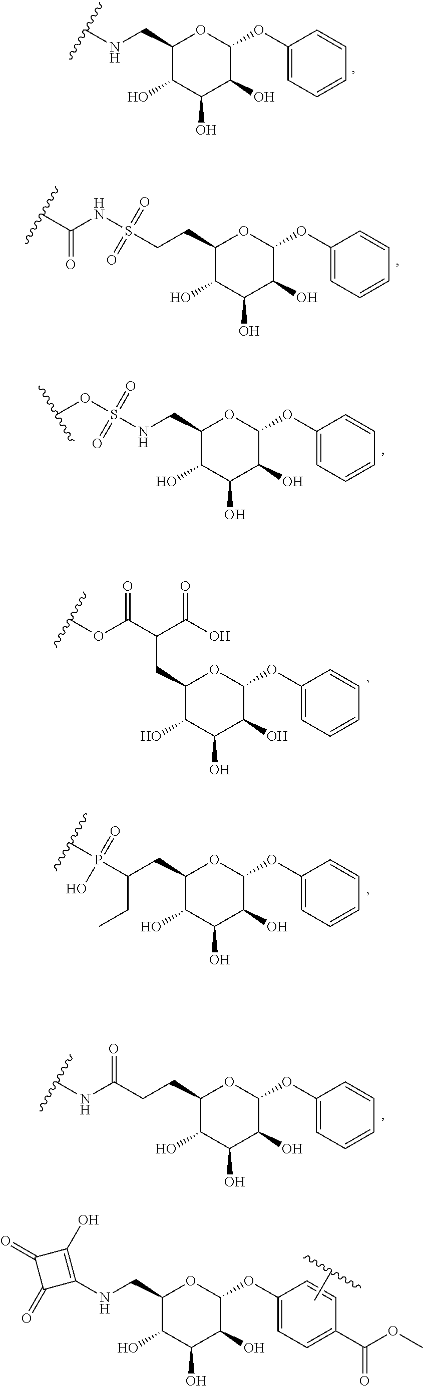

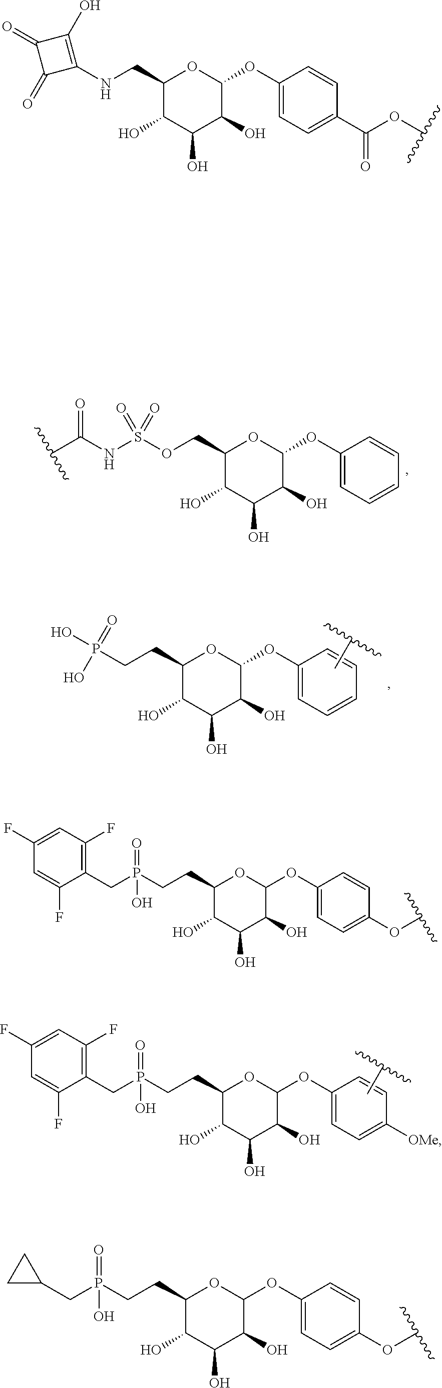

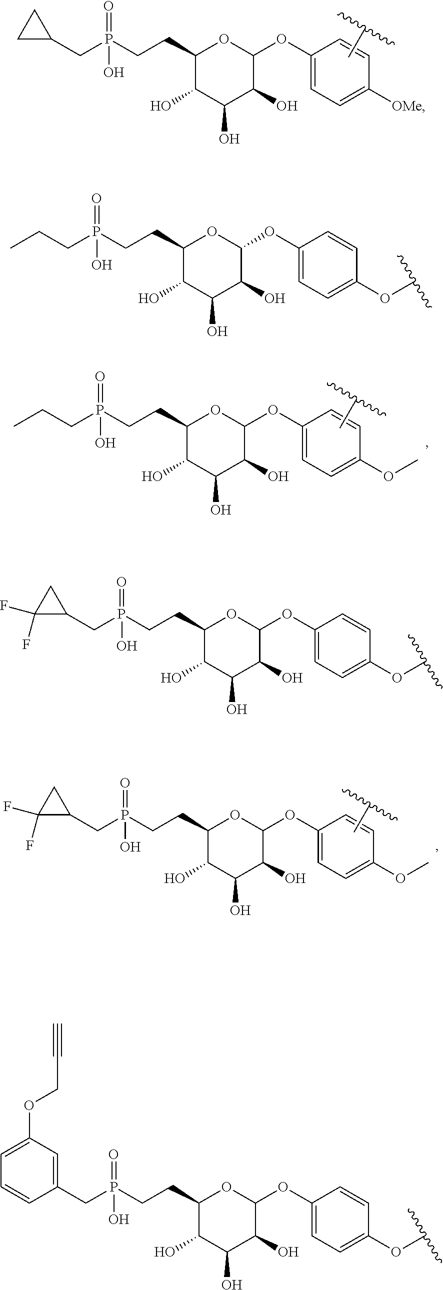

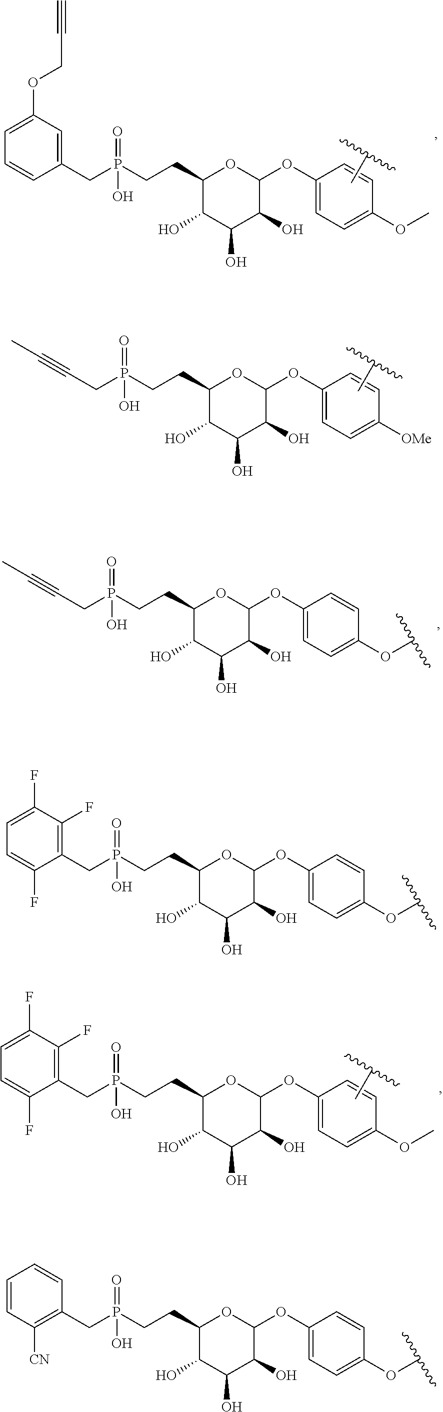

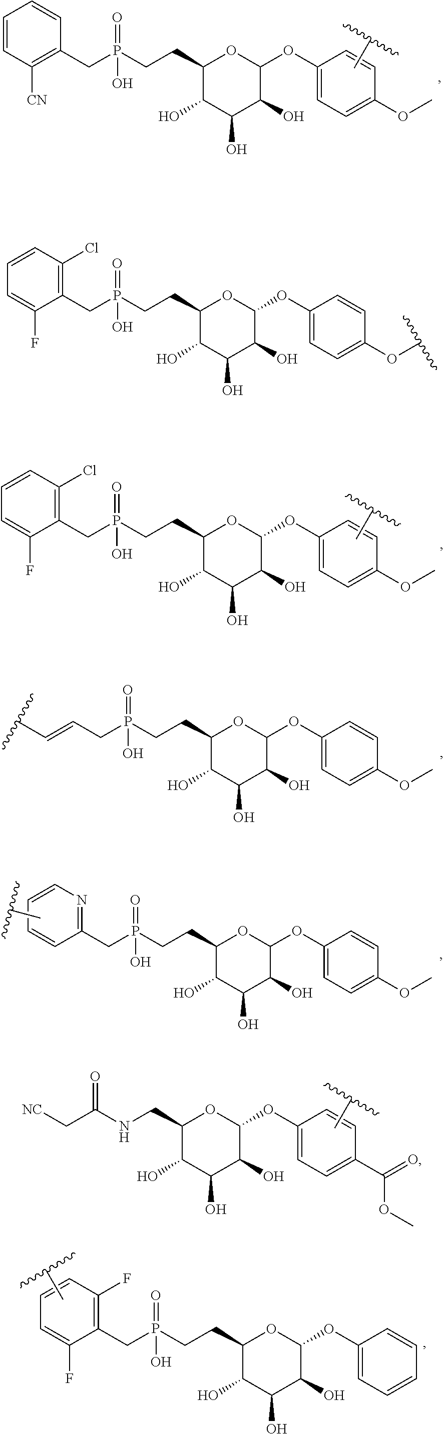

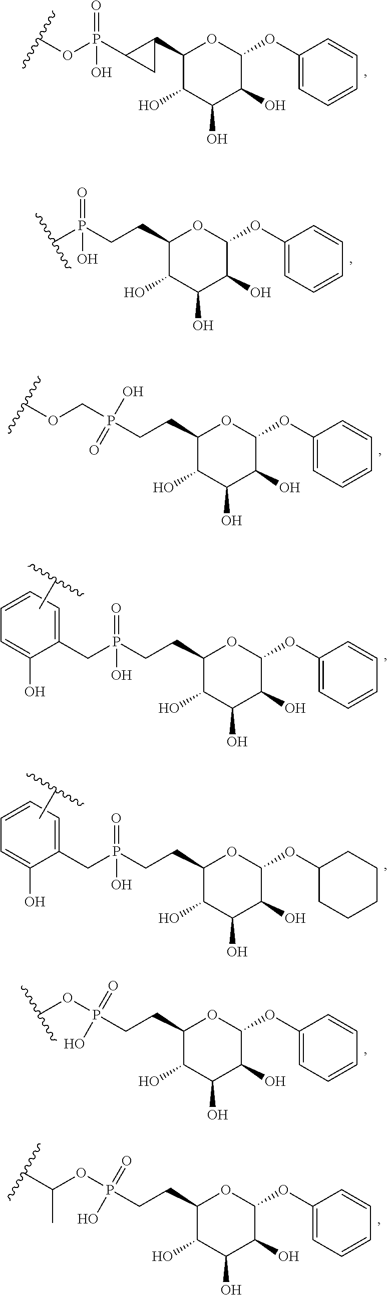

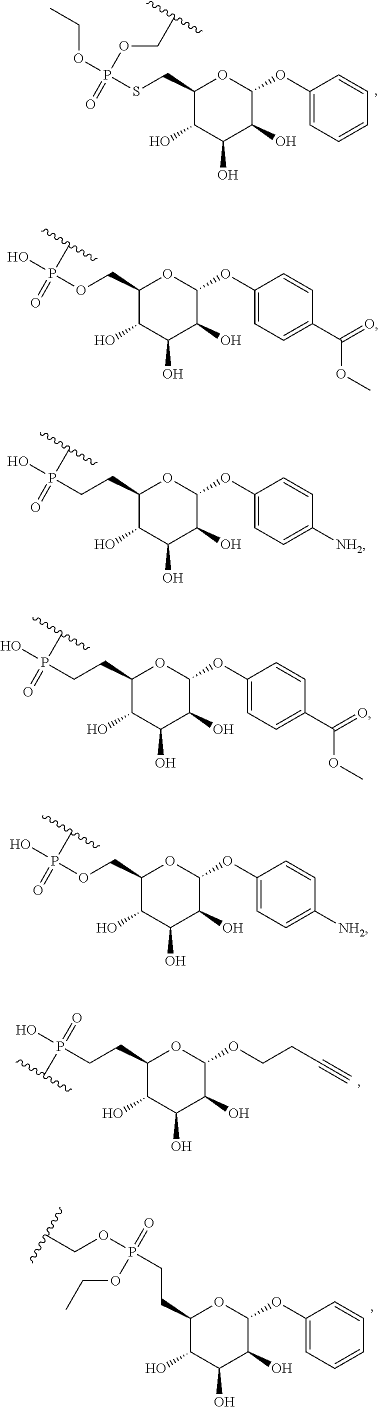

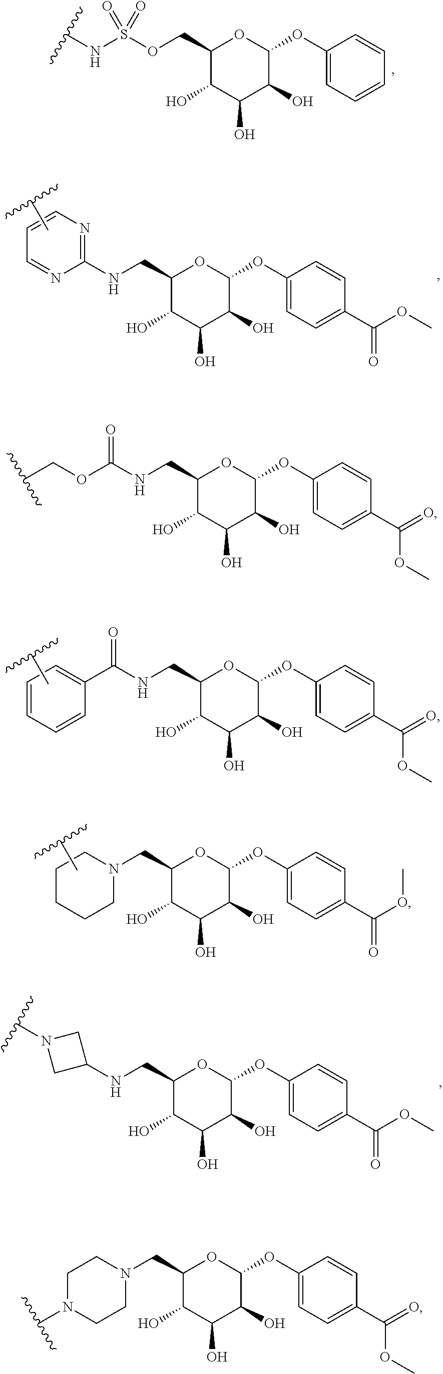

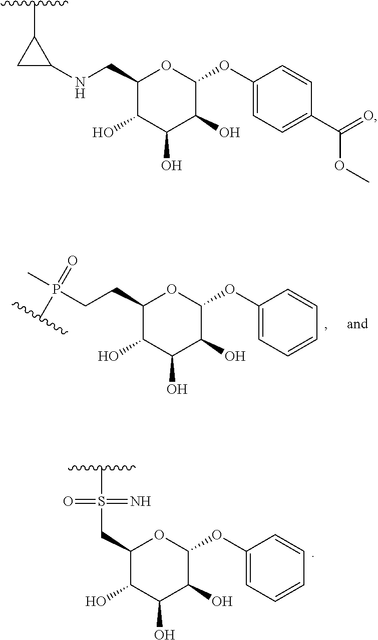

In certain aspects, an Extracellular Protein Degrader is of Formula:

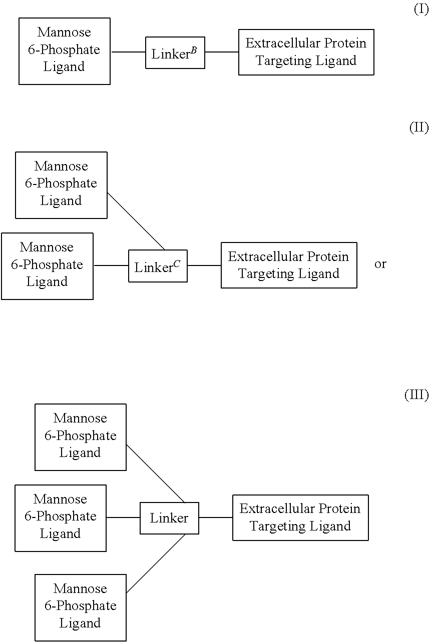

or a pharmaceutically acceptable salt thereof;
wherein:
Mannose 6-Phosphate Ligand is selected from:

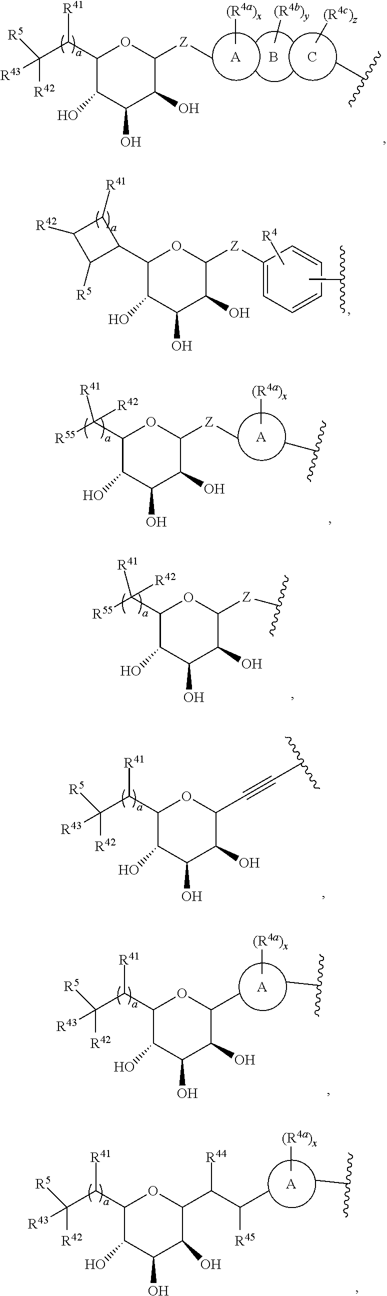

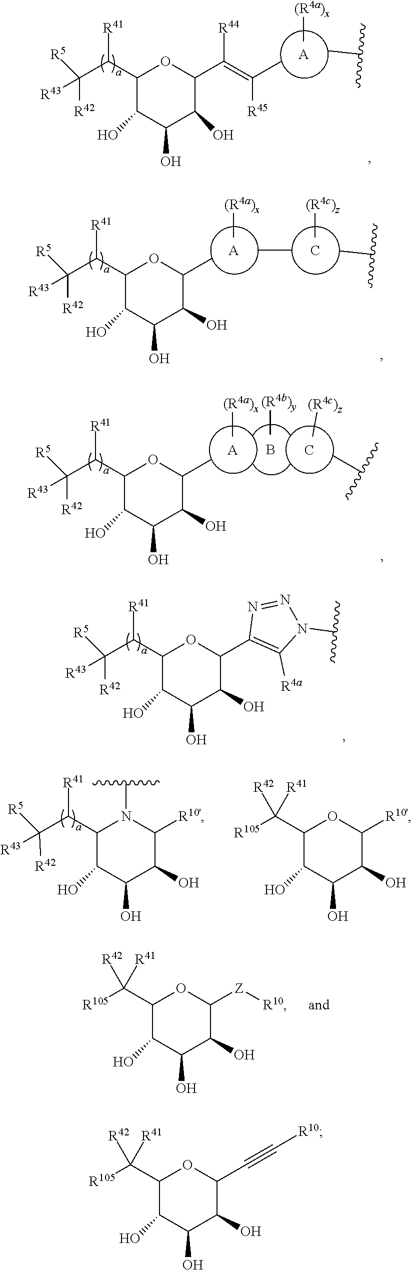

a, x, y, and z are independently 0, 1, 2, or 3;
Z is NR⁸, O, S, NC(O)R³, or CR⁴R⁸,
\textcircled{A} is cycloalkyl, heterocycle, aryl or heteroaryl;
\textcircled{B} is a nonaromatic cycloalkyl or heterocycle;
\textcircled{C} is aryl or heteroaryl;
for example, a compound of structure

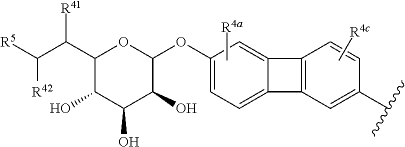

is a compound of the formula

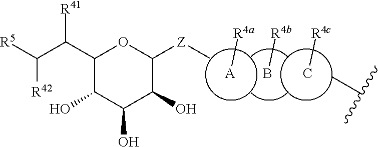

wherein \textcircled{A} is phenyl, \textcircled{B} is cyclobutyl, and \textcircled{C} is phenyl;
R³ at each occurrence is independently selected from hydrogen, alkyl, haloalkyl, arylalkyl, heteroarylalkyl, alkenyl, alkynyl, aryl, heteroaryl, heterocycle, —OR⁸, and —NR⁸R⁹;
R⁴, R^{4a}, R^{4b}, and R^{4c} are independently selected at each occurrence from hydrogen, F, Cl, Br, I, alkyl, haloalkyl, arylalkyl, heteroarylalkyl, alkenyl, alkynyl, aryl, heteroaryl, heterocycle, cyano, —OR⁶, —NR⁶R⁷, C(O)R³, S(O)R³, C(S)R³, and S(O)₂R³;
R⁴¹, R⁴², R⁴³, R⁴⁴, and R⁴⁵ are independently selected at each occurrence from hydrogen, alkyl, haloalkyl, F, arylalkyl, heteroarylalkyl, alkenyl, alkynyl, aryl, heteroaryl, and heterocycle;
R⁵⁰ is hydrogen, alkyl, haloalkyl, arylalkyl, heteroarylalkyl, alkenyl, alkynyl, aryl, heteroaryl, heterocycle, —OR⁶, or—NR⁶R⁷,
R⁵ is selected from

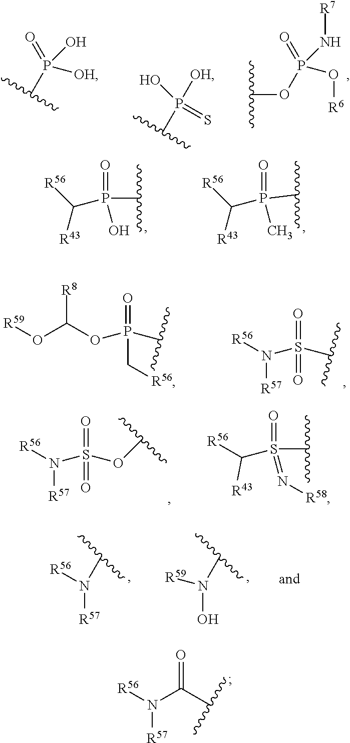

in other embodiments R⁵ is R⁵⁵,
R⁵⁵ is selected from

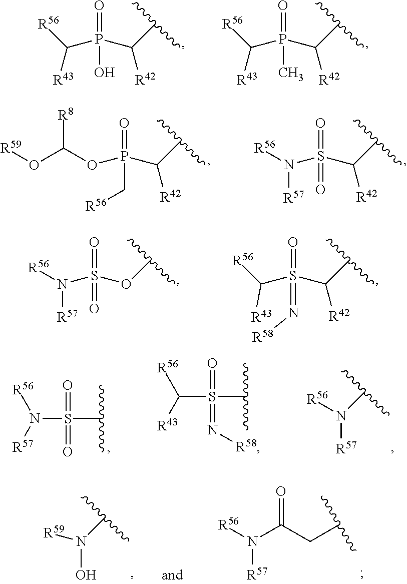

R⁵⁶ is independently selected from hydrogen, alkyl, alkenyl, alkynyl, haloalkyl, —O-alkyl, hydroxyl, cyano, heterocyclyl, aryl, and heteroaryl wherein each group except for hydrogen may optionally be substituted with 1, 2, or 3 independently selected R^{9a} substituents as allowed by valence;
R⁵⁷ is selected from hydrogen, alkyl, alkenyl, alkynyl, haloalkyl, —O-alkyl, aryl, heteroaryl, C(O) H, C(O) alkyl, C(O) alkenyl, C(O) alkynyl, C(O) haloalkyl, C(O) aryl, C(O)heteroaryl, C(O)O-alkyl, C(O)O-alkenyl, C(O)O-alkynyl, C(O)O-haloalkyl, C(O)O-aryl, C(O)O-heteroaryl and

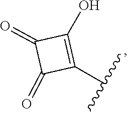 [SMILES: *c1c(O)c(=O)c1=O]

wherein each group except for hydrogen may optionally be substituted with 1, 2, or 3 independently selected R⁹% substituents as allowed by valence;
or R⁵⁶, R⁵⁷, and the nitrogen to which they are attached form a 5 to 7 membered heterocyclic ring such as but not limited to piperidine and piperazine which can be optionally substituted by alkyl, heterocycle, aryl, and heteroaryl, for example, a compound of the structure

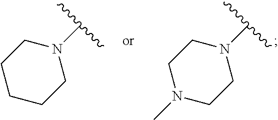

R⁵⁸ is selected from hydrogen, alkyl, C(O) alkyl, C(O)O-alkyl, C(O) aryl, C(O)heteroaryl, C(O)O-aryl, C(O)O-arylalkyl, and C(O)O-heteroaryl, wherein each group may optionally be substituted with 1, 2, or 3 independently selected R^{9c} substituents as allowed by valence;
R⁵⁹ is selected from C(O) alkyl, C(O)O-alkyl, C(O) aryl, C(O)heteroaryl, C(O)O-aryl, and C(O)O-heteroaryl, wherein each group may optionally be substituted with 1, 2, or 3 independently selected R^{9d} substituents as allowed by valence;
R⁶ and R⁷ are independently selected at each occurrence from hydrogen, alkyl, arylalkyl, heteroarylalkyl, alkenyl, alkynyl, aryl, haloalkyl, heteroaryl, heterocycle, -alkyl—OR⁸, -alkyl—NR⁸R⁹, C(O)R³, S(O)R³, C(S)R³, and S(O)₂R³;
R⁸ is independently selected at each occurrence from hydrogen, alkyl, arylalkyl, heteroarylalkyl, alkenyl, alkynyl, aryl, heteroaryl, heterocycle, C(O)R³, S(O)R³, C(S)R³, and S(O)₂R³;
R⁹ is independently selected at each occurrence from hydrogen, alkyl, arylalkyl, heteroarylalkyl, alkenyl, alkynyl, aryl, heteroaryl, and heterocycle;
R^{9a}, R^{9b}, R^{9c}, and R^{9d} are independently selected at each occurrence from hydrogen, F, Cl, Br, I, alkyl, haloalkyl, arylalkyl, heteroarylalkyl, alkenyl, alkynyl, aryl, heteroaryl, heterocycle, cyano, —OR⁶, —NR⁶R⁷, C(O)R³, S(O)R³, C(S)R³, and S(O)₂R³;
R¹⁰ is selected from hydrogen, alkyl, aryl, heteroaryl, arylalkyl, heteroarylalkyl, alkyl-O-alkyl, C(O) aryl, C(O) alkyl, C(O) arylalkyl, C(O)heteroarylalkyl, each of which R¹⁰ except hydrogen is optionally substituted with 1, 2, 3, or 4 substituents selected from alkyl, alkenyl, alkynyl, haloalkyl, —OR⁶, F, Cl, Br, I, —NR⁶R⁷, heterocycle, heteroaryl, aryl, cyano, nitro, hydroxyl, amide, —SR³, —S(O)(NR⁶)R³, —NR^{8c}(O)R³, —C(O)NR⁶R⁷, —C(O)OR³, and —C(O)R³;
or R¹⁰ is

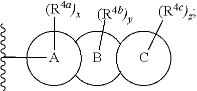

R¹⁰′ is selected from hydrogen, alkyl, aryl, heteroaryl, arylalkyl, heteroarylalkyl, each of which R¹⁰′ except hydrogen is optionally substituted with 1, 2, 3, or 4 substituents selected from alkyl, alkenyl, alkynyl, haloalkyl, —OR⁶, F, Cl, Br, I, —NR⁶R⁷, heterocycle, heteroaryl, aryl, cyano, nitro, hydroxyl, amide, —SR³, —S(O)(NR⁶)R³, —NR⁸C(O)R³, —C(O)NR⁶R⁷, —C(O)OR³, and —C(O)R³;′
R¹⁰⁵ is selected from

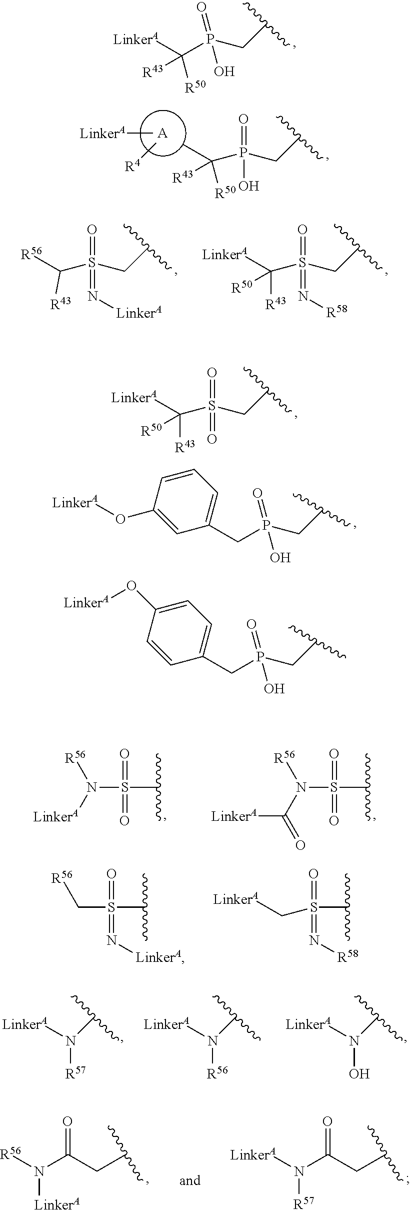

each Linker^{A} is a bond or a moiety that covalently links the sugar (e.g. the C₁ or C₅ position) to Linker^{B};
Linker^{B} is a bond or a moiety that covalently links Linker^{A} to an Extracellular Protein Targeting Ligand;
Linker^{C} is a moiety that covalently links Linker^{A} to an Extracellular Protein Targeting Ligand;
Linker^{D} is a moiety that covalently links Linker^{A} to an Extracellular Protein Targeting Ligand;
Extracellular Protein Targeting Ligand is a chemical moiety that binds to the targeted disease-mediating extracellular protein; and
wherein the optional substituents are selected such that a stable compound results for example a compound of Formula I, Formula II, or Formula III with a shelf stability of at least 1, 2, 3, 4, 5, 6 or more months.

In other embodiments a compound of Formula IV is provided:

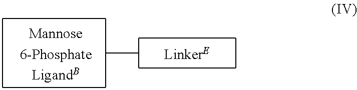

or a salt thereof;
wherein:
Mannose 6-Phosphate Ligand® is selected from:

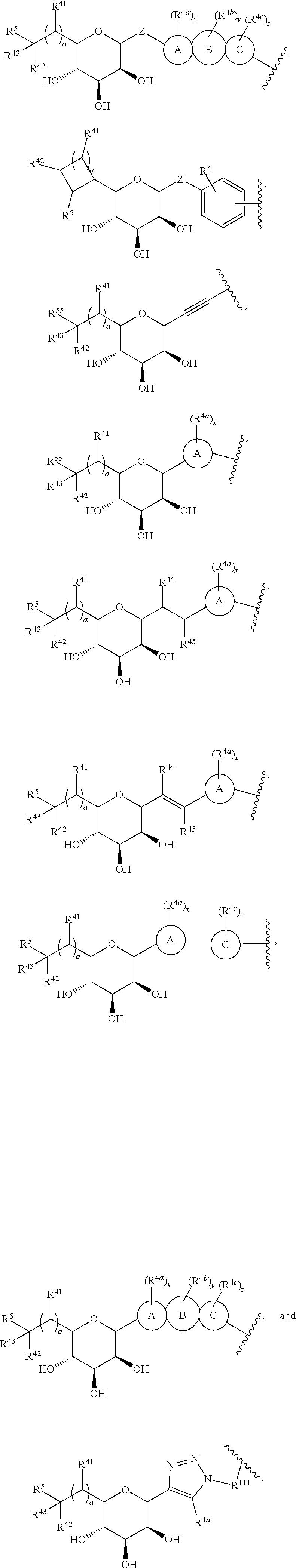

### VII. Embodiments of the Linker

In non-limiting embodiments, Linker^{A} and Linker^{B} are independently selected from:
wherein:

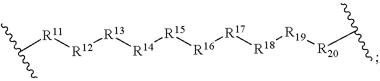

R¹¹, R¹², R¹³, R¹⁴, R¹⁵, R¹⁶, R¹⁷, R¹⁸, R¹⁹, and R²⁰ are independently at each occurrence selected from the group consisting of a bond, alkyl, —C(O)—, —C(O)O—, —OC(O)—, —SO₂—, —S(O)—, —C(S)—, —C(O)NR⁶—, —NR⁶C(O)—, —C(O)N(OH)—, —N(OH)C(O)—, —O—, —S—, —NR⁶—, —C(R²¹R²¹)—, —S(O)(═N—R⁴⁴)—, —S(O)(═NR), —P(O)(R³)O—, —P(O)(R³)—, a divalent residue of a natural or unnatural amino acid, alkenyl, alkynyl, haloalkyl, alkoxy, aryl, heterocycle, heteroaryl, —CH₂CH₂—[O—(CH₂)₂]ₙ—O—, —CH₂CH₂—[O—(CH₂)₂]ₙ—NR⁶—, —CH₂CH₂—[O—(CH₂)₂]ₙ—, -[—(CH₂)₂—O—]n—, —[O—(CH₂)₂]ₙ—, —[O—CH(CH₃)C(O)]ₙ—, —[C(O)—CH(CH₃)—O]ₙ—, —[O—CH₂C(O)]ₙ—, —[C(O)—CH₂—O]ₙ—, a divalent residue of a fatty acid, a divalent residue of an unsaturated or saturated mono- or di-carboxylic acid; each of which is optionally substituted with 1, 2, 3, or 4 substituents independently selected from R²¹;
n is independently selected at each instance from 0, 1, 2, 3, 4, 5, 6, 7, 8, 9, or 10;
R²¹ is independently at each occurrence selected from the group consisting of hydrogen, alkyl, alkenyl, alkynyl, F, Cl, Br, I, hydroxyl, alkoxy, azide, amino, cyano, —NR⁶R⁷, —NR⁸SO₂R³, —NR⁸S(O)R³, haloalkyl, aryl, heteroaryl, and heterocycle;
and the remaining variables are as defined herein.

In certain embodiments Linker^{A} is bond and Linker^{B} is

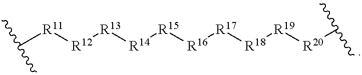

In certain embodiments Linker^{B} is bond and Linker^{A} is

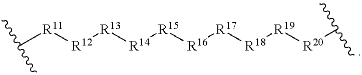

In certain embodiments, a divalent residue of an amino acid is selected from

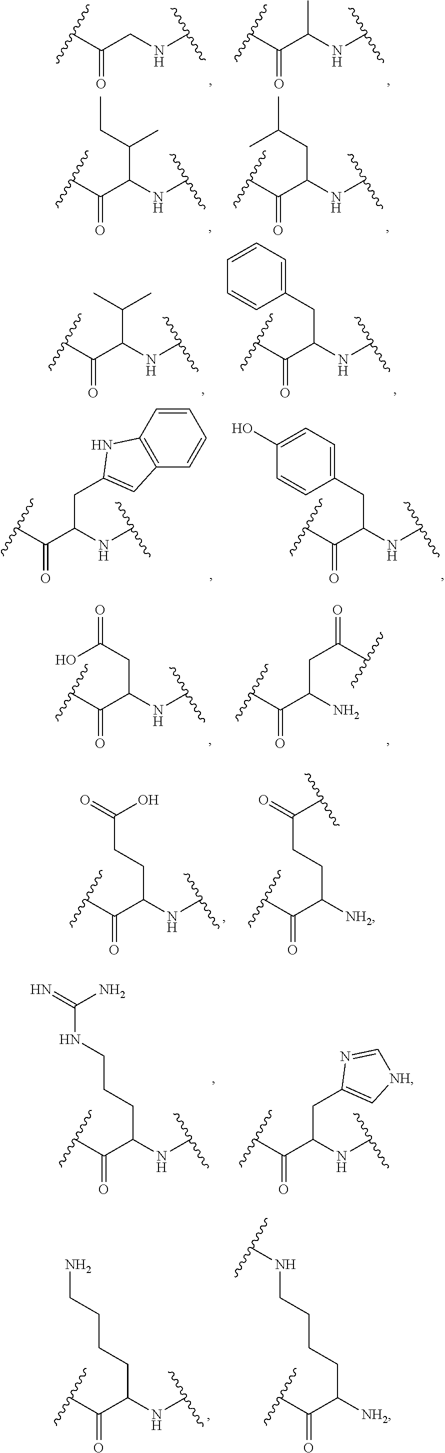

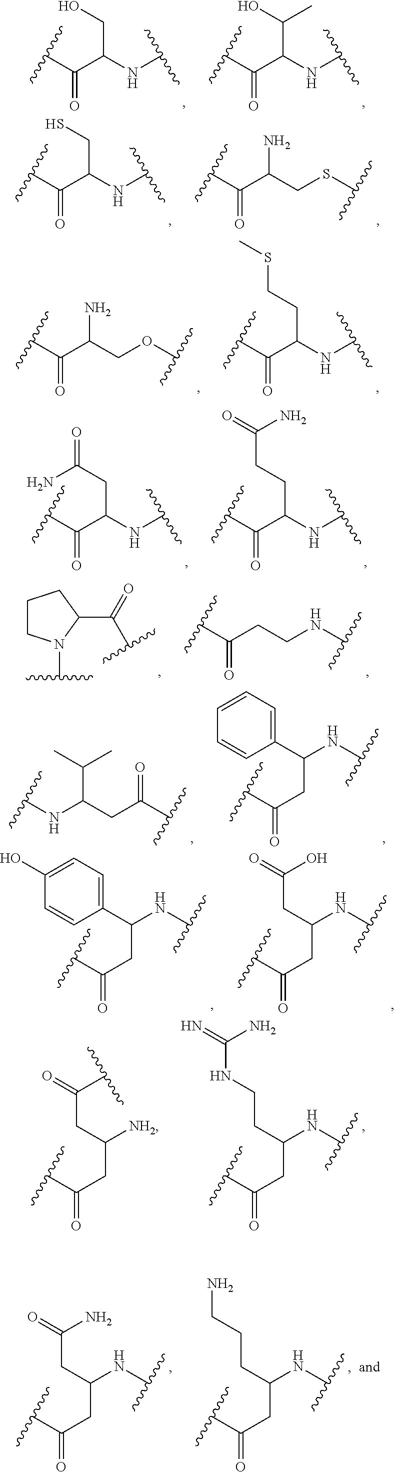

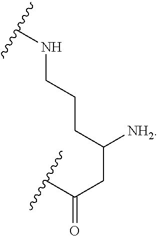 [SMILES: *NCCCC(N)CC(*)=O]

wherein the amino acid can be oriented in either direction and wherein the amino acid can be in the L- or D-form or a mixture thereof.

In certain embodiments, a divalent residue of a dicarboxylic acid is generated from a nucleophilic addition reaction:

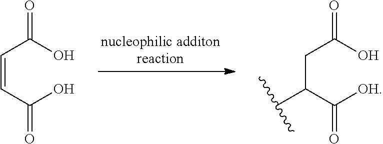 [SMILES: O=C(O)/C=C\C(=O)O ; *C(CC(=O)O)C(=O)O]

Non-limiting embodiments of a divalent residue of a dicarboxylic acid generated from a nucleophilic addition reaction include:

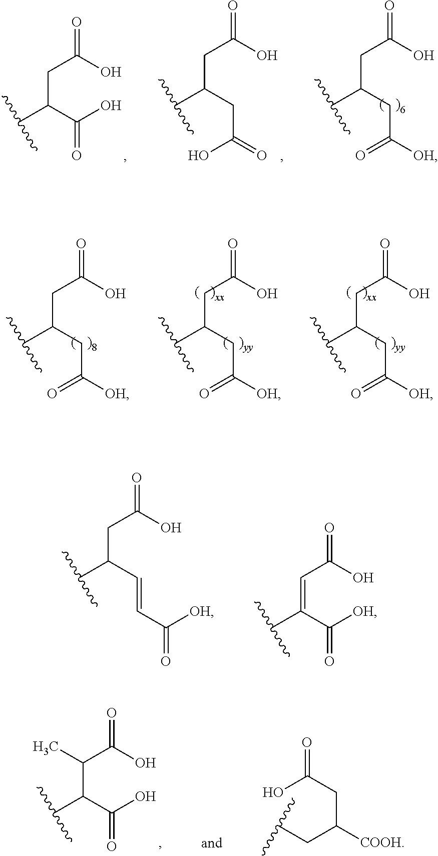

In certain embodiments, a divalent residue of a dicarboxylic acid is generated from a condensation reaction:

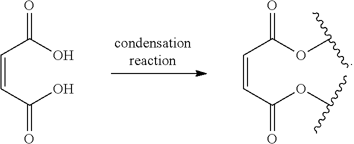 [SMILES: O=C(O)/C=C\C(=O)O ; *OC(=O)/C=C\C(=O)O*]

Non-limiting embodiments of a divalent residue of a dicarboxylic acid generated from a condensation include:

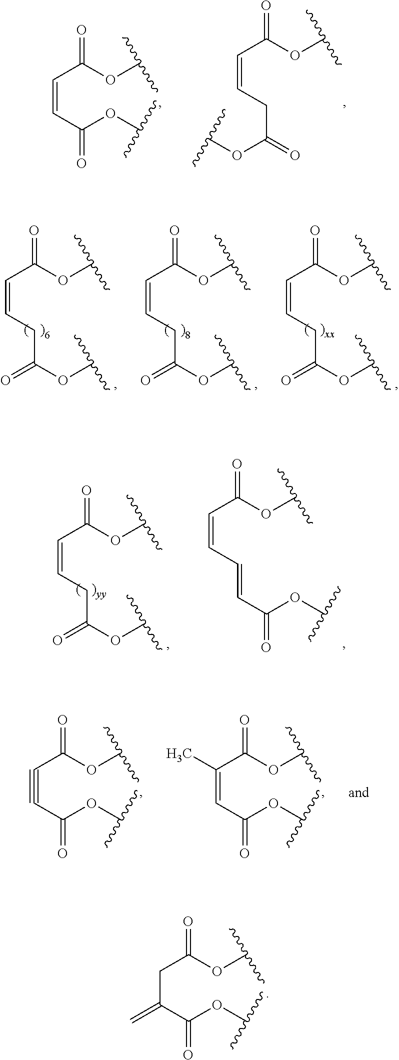

Non-limiting embodiments of a divalent residue of a saturated dicarboxylic acid include:

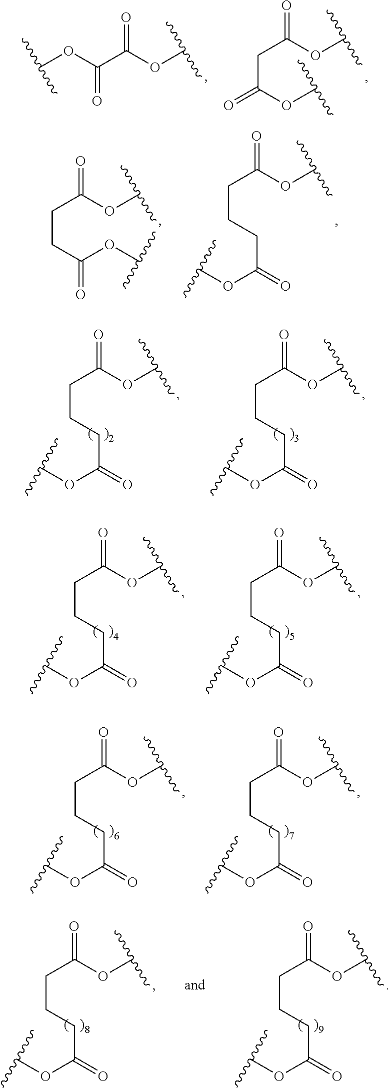

Non-limiting embodiments of a divalent residue of a saturated dicarboxylic acid include:

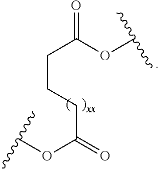

Non-limiting embodiments of a divalent residue of a saturated monocarboxylic acid is selected from butyric acid (—OC(O)(CH₂)₂CH₂—), caproic acid (—OC(O)(CH₂)₄CH₂—), caprylic acid (—OC(O)(CH₂)₅CH₂—), capric acid (—OC(O)(CH₂)₈CH₂—), lauric acid (—OC(O)(CH₂)₁₀CH₂—), myristic acid (—OC(O)(CH₂)₁₂CH₂—), pentadecanoic acid (—OC(O)(CH₂)₁₃CH₂—), palmitic acid (—OC(O)(CH₂)₁₄CH₂—), stearic acid (—OC(O)(CH₂)₁₆CH₂—), behenic acid (—OC(O)(CH₂)₂₀CH₂—), and lignoceric acid (—OC(O)(CH₂)₂₂CH₂—);

Non-limiting embodiments of a divalent residue of a fatty acid include residues selected from linoleic acid, palmitoleic acid, vaccenic acid, paullinic acid, oleic acid, elaidic acid, gondoic acid, gadoleic acid, nervonic acid, myristoleic acid, and erucic acid:

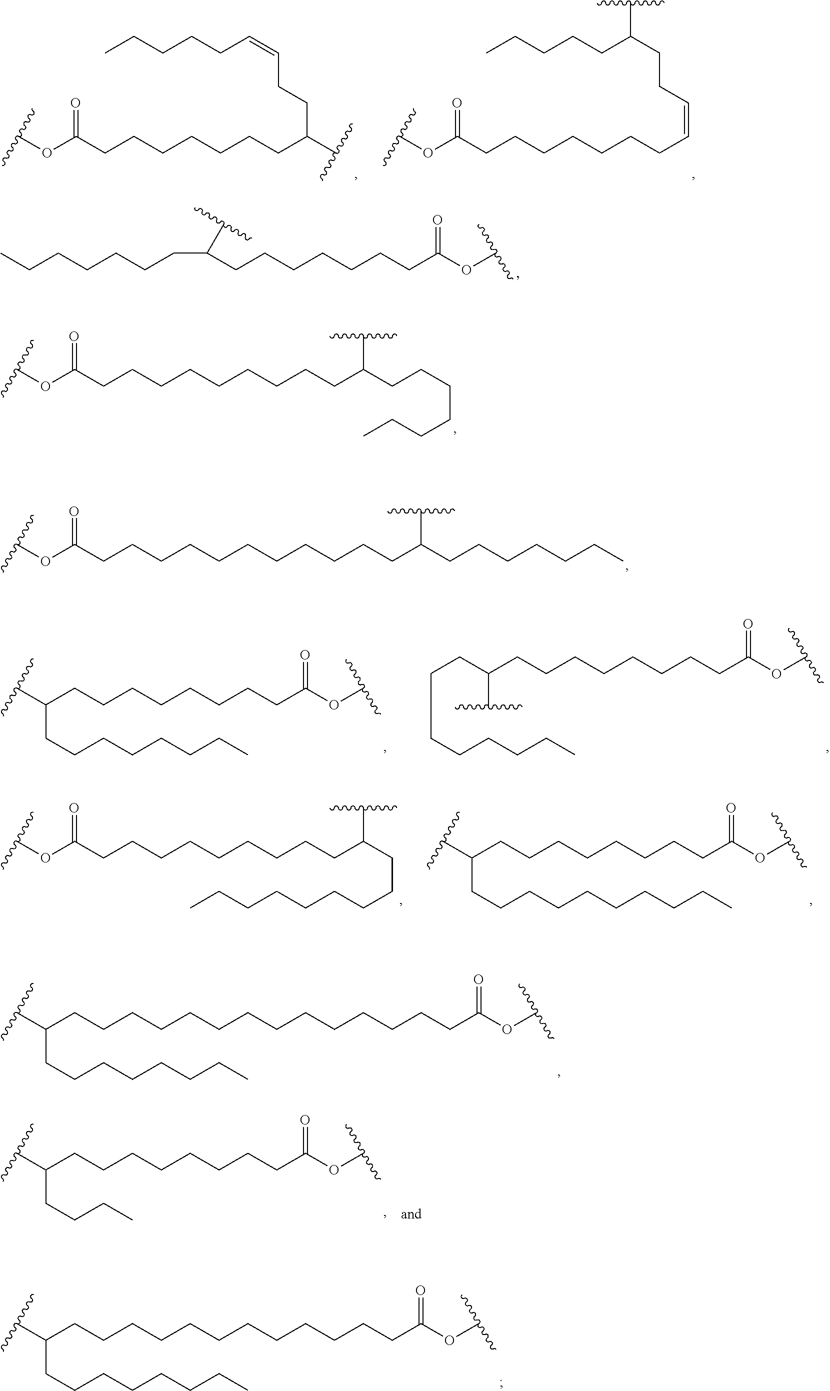

Non-limiting embodiments of a divalent residue of a fatty acid is selected from linoleic acid (—C(O)(CH₂)₇(CH)₂CH₂(CH)₂(CH₂)₄CH₂—), docosahexaenoic acid (—C(O)(CH₂)₂(CHCHCH₂)₆CH₂—), eicosapentaenoic acid (—C(O)(CH₂)₃(CHCHCH₂)₅CH₂—), alpha-linolenic acid (—C(O)(CH₂)₇(CHCHCH₂)₃CH₂—) stearidonic acid (—C(O)(CH₂)₄(CHCHCH₂)₄CH₂—), γ-linolenic acid (—C(O)(CH₂)₄(CHCHCH₂)₃(CH₂)₃CH₂—), arachidonic acid (—C(O)(CH₂)₃, (CHCHCH₂)₄(CH₂)₄CH₂—), docosatetraenoic acid (—C(O)(CH₂)₅(CHCHCH₂)₄(CH₂)₄CH₂—), palmitoleic acid (—C(O)(CH₂)₇CHCH(CH₂)₅CH₂—), vaccenic acid (—C(O)(CH₂)₇CHCH(CH₂)₅CH₂—), paullinic acid (—C(O)(CH₂)₁₁CHCH(CH₂)₅CH₂—), oleic acid (—C(O)(CH₂)₇CHCH(CH₂)₇CH₂—), elaidic acid (—C(O)(CH₂)₇CHCH(CH₂)₇CH₂—), gondoic acid (—C(O)(CH₂)₉CHCH(CH₂)₇CH₂—), gadoleic acid (—C(O)(CH₂)₇CHCH(CH₂)₉CH₂—), nervonic acid (—C(O)(CH₂)₁₃CHCH(CH₂)₇CH₂—), mead acid (—C(O)(CH₂)₃(CHCHCH₂)₃(CH₂)₆CH₂—), myristoleic acid (—C(O)(CH₂)₇CHCH(CH₂)₃CH₂—), and erucic acid (—C(O)(CH₂)₁₁CHCH(CH₂)₇CH₂—).

In certain embodiments Linker^{C} is selected from:

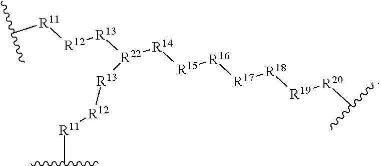

wherein:
R²² is independently at each occurrence selected from the group consisting of alkyl, —C(O)N—, —NC(O)—, —N—, —C(R²¹)—, —P(O)O—, —P(O)—, —P(O)(NR⁶R⁷)N—, alkenyl, haloalkyl, aryl, heterocycle, and heteroaryl, each of which is optionally substituted with 1, 2, 3, or 4 substituents independently selected from R²¹;
and the remaining variables are as defined herein.

In certain embodiments Linker^{D} is selected from:

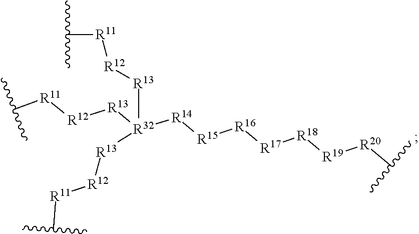

wherein

R³² is independently at each occurrence selected from the group consisting of alkyl, N⁺X⁻, —C—, alkenyl, haloalkyl, aryl, heterocycle, and heteroaryl, each of which is optionally substituted with 1, 2, 3, or 4 substituents independently selected from R²¹;
X⁻ is an anionic group, for example Br⁻ or Cl⁻.

In certain embodiments, the Linker_{A} is selected from

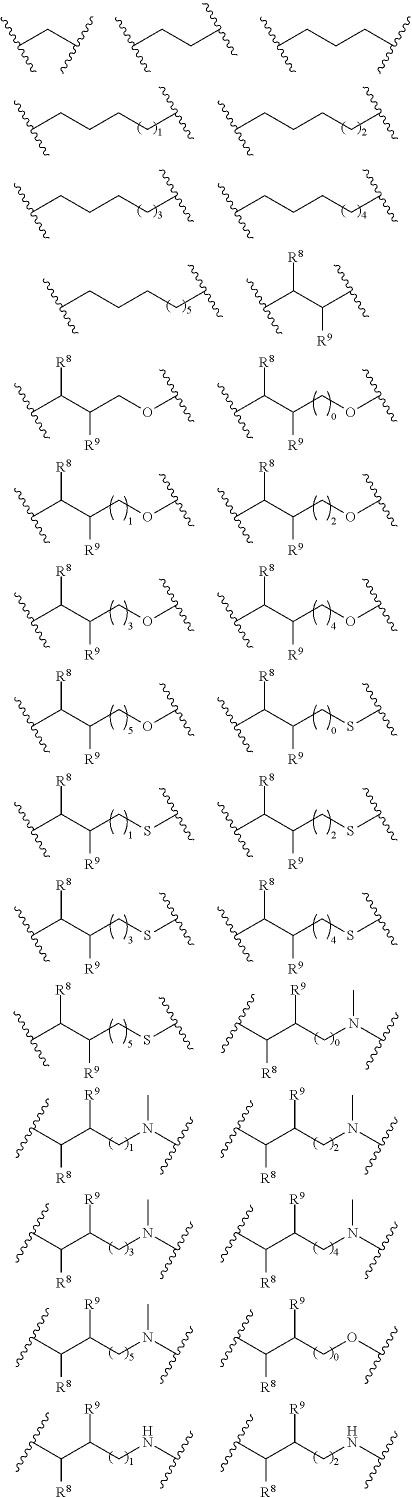

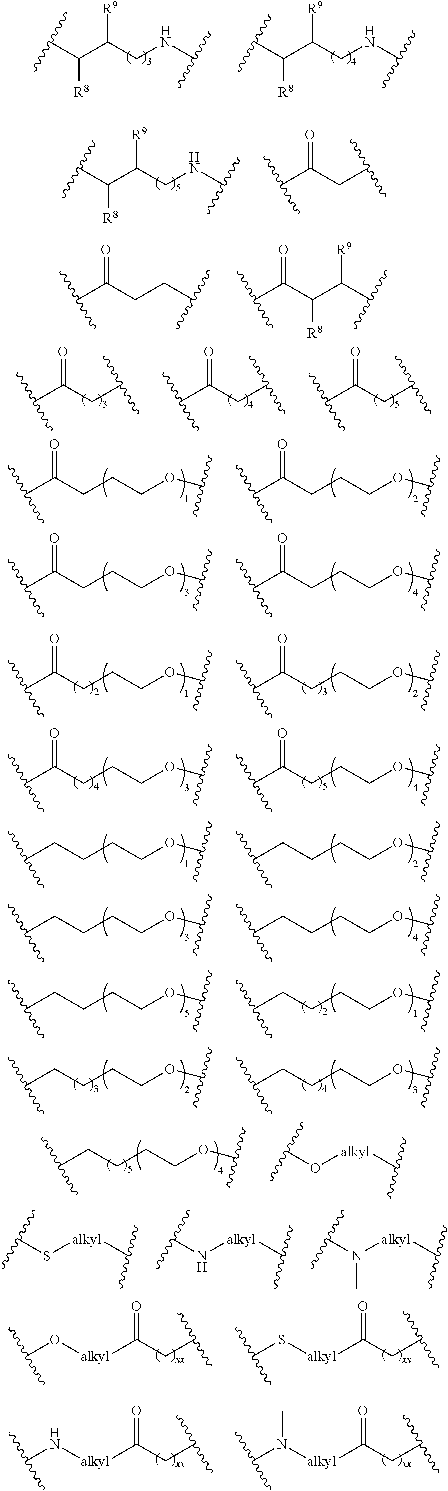

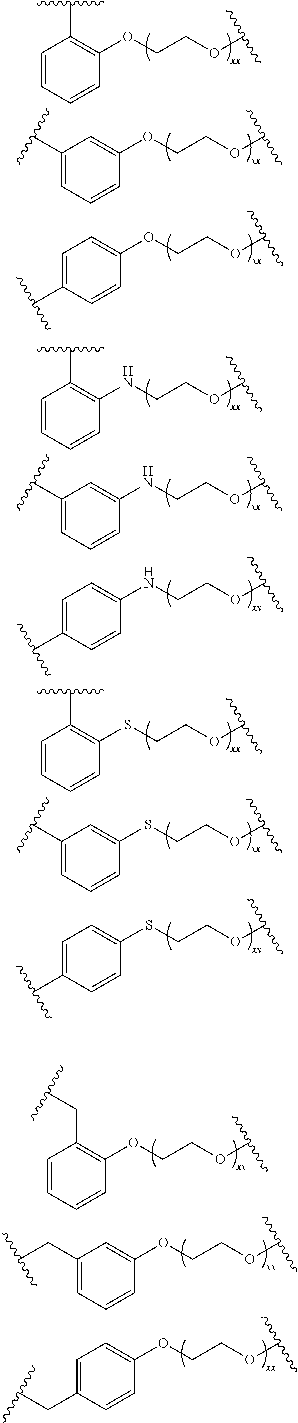

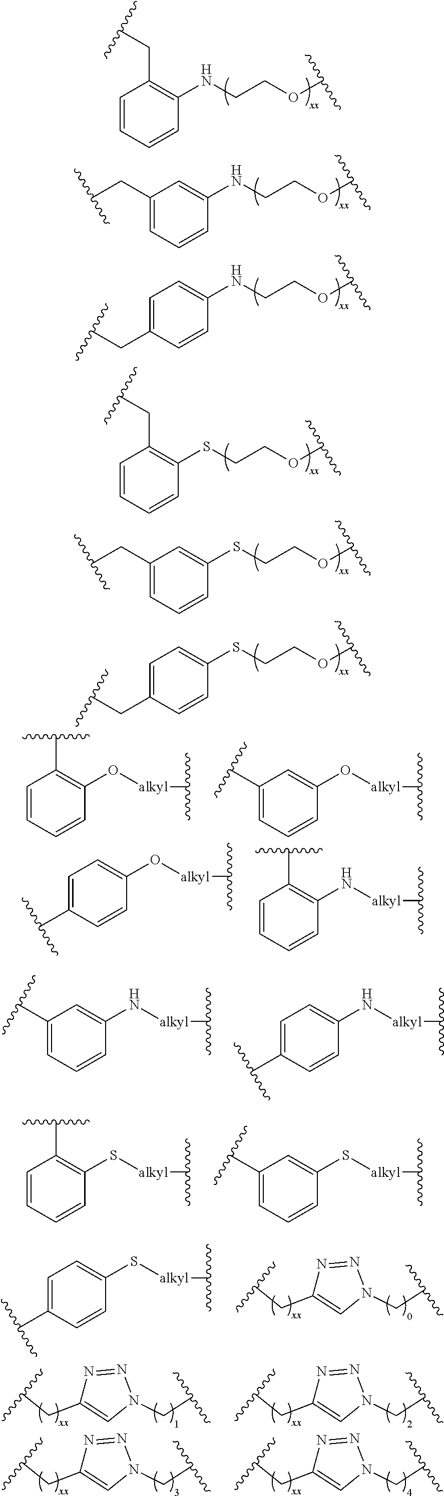

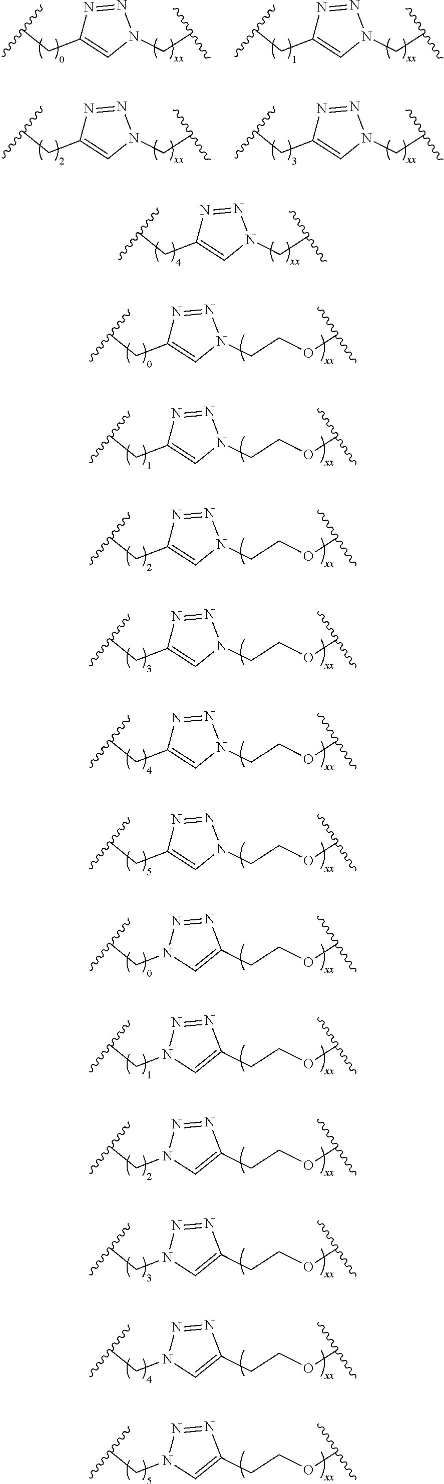

In certain embodiments Linker^{A} is selected from:

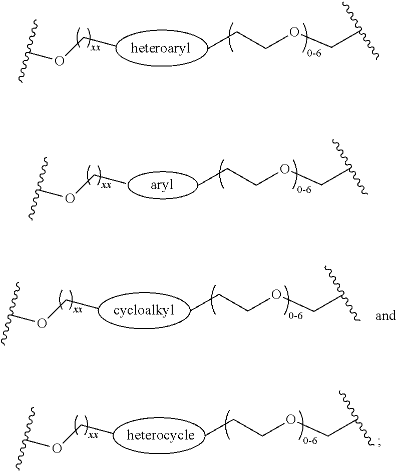

wherein each heteroaryl, heterocycle, cycloalkyl, and aryl can optionally be substituted with 1, 2, 3, or 4 of any combination of halogen, alkyl, haloalkyl, aryl, heteroaryl, heterocycle, or cycloalkyl, as allowed by valence.

In certain embodiments Linker^{A} is selected from:

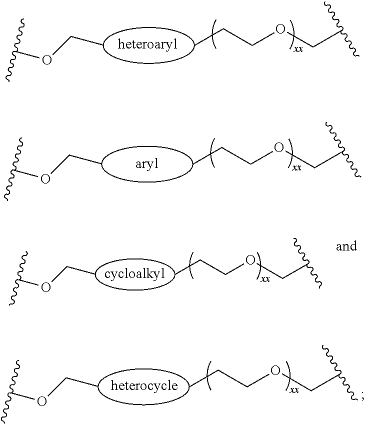

wherein each heteroaryl, heterocycle, cycloalkyl, and aryl can optionally be substituted with 1, 2, 3, or 4 of any combination of halogen, alkyl, haloalkyl, aryl, heteroaryl, heterocycle, or cycloalkyl, as allowed by valence.

In certain embodiments Linker^{A} is selected from:

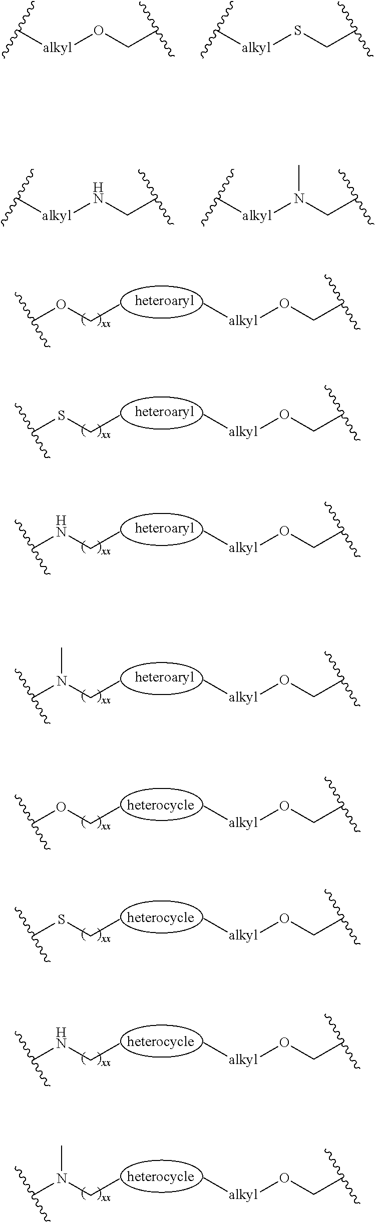

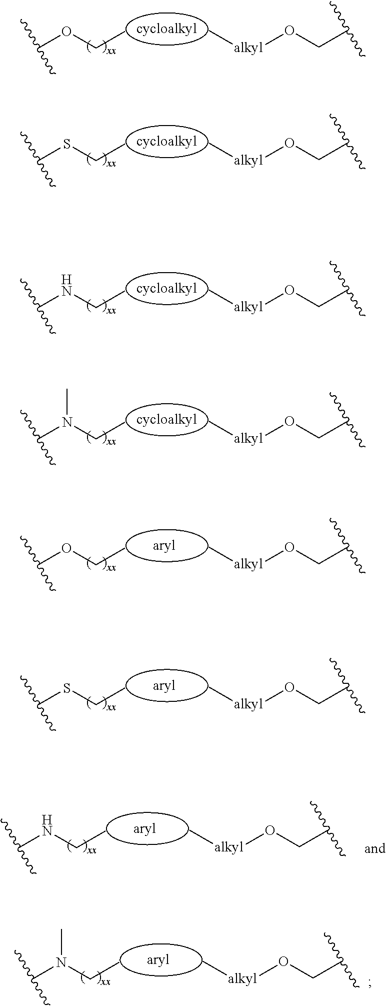

wherein each heteroaryl, heterocycle, cycloalkyl, and aryl can optionally be substituted with 1, 2, 3, or 4 of any combination of halogen, alkyl, haloalkyl, aryl, heteroaryl, heterocycle, or cycloalkyl, as allowed by valence.

In certain embodiments Linker® is selected from:

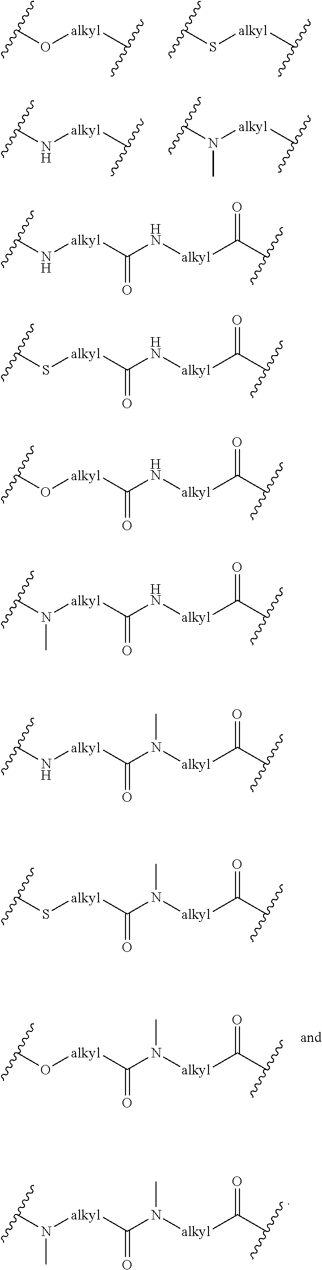

In certain embodiments Linker® is selected from:

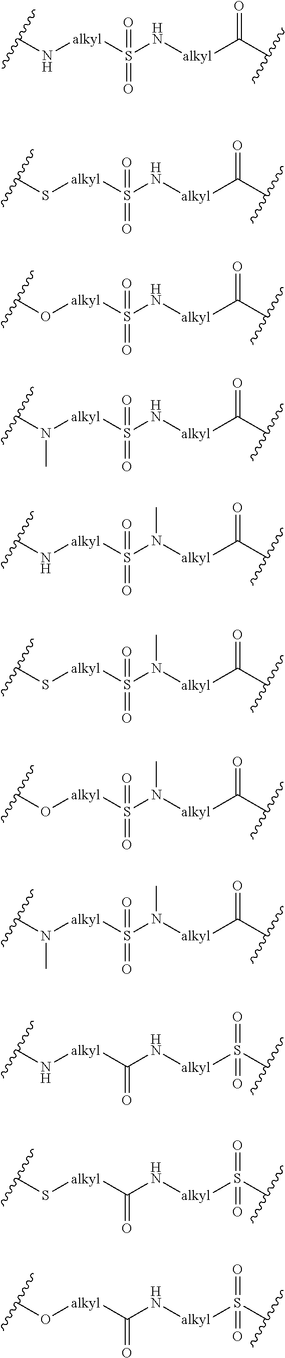

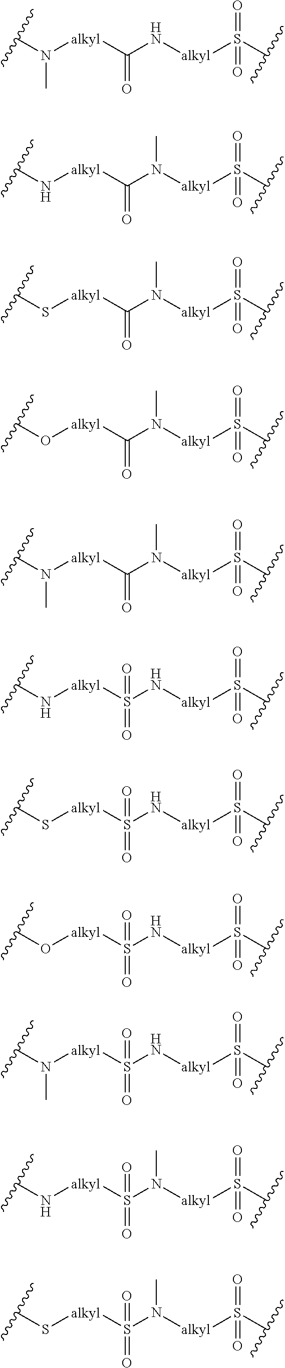

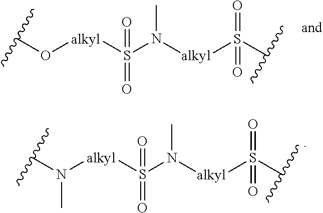

In certain embodiments Linker^{B}, Linker^{C}, or Linker^{D} is selected from:

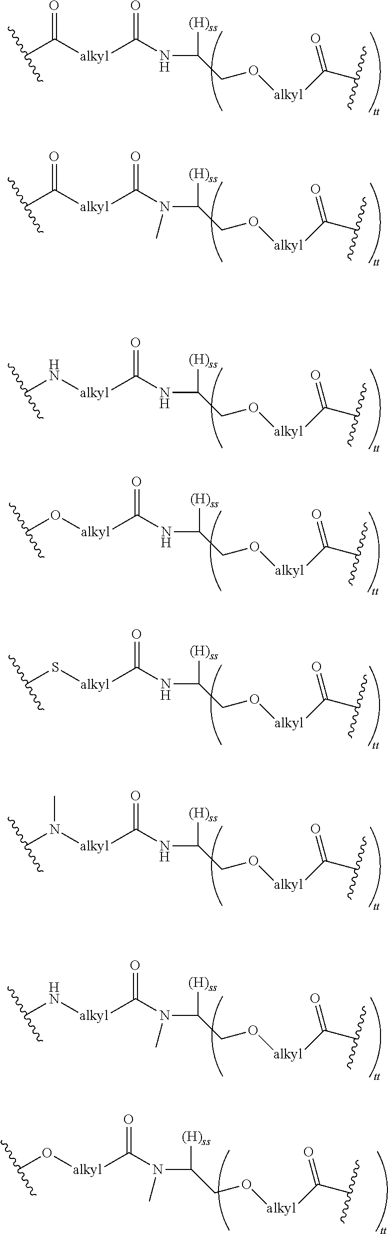

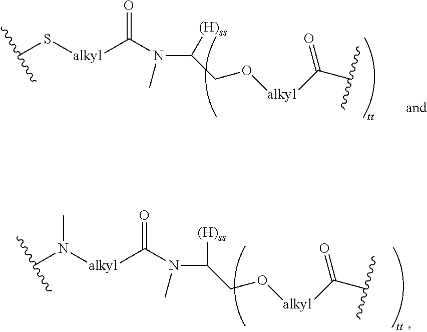

wherein tt is independently selected from 1, 2, or 3 and ss is 3 minus tt.

In certain embodiments Linker^{B}, Linker^{C}, or Linker^{D} is selected from:

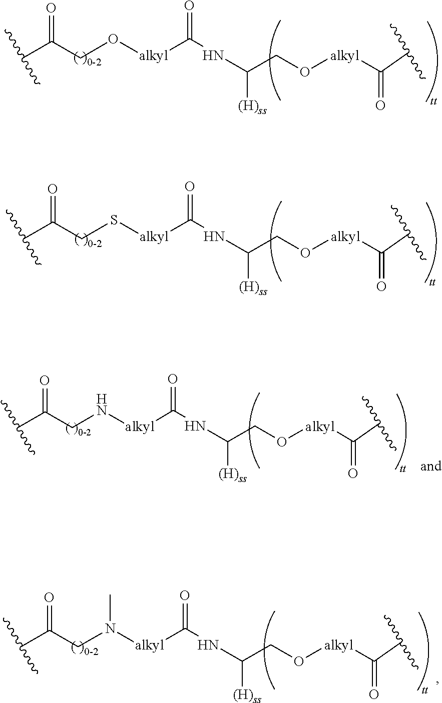

wherein tt and ss are as defined herein.

In certain embodiments Linker^{B}, Linker^{C}, or Linker^{D} is selected from:

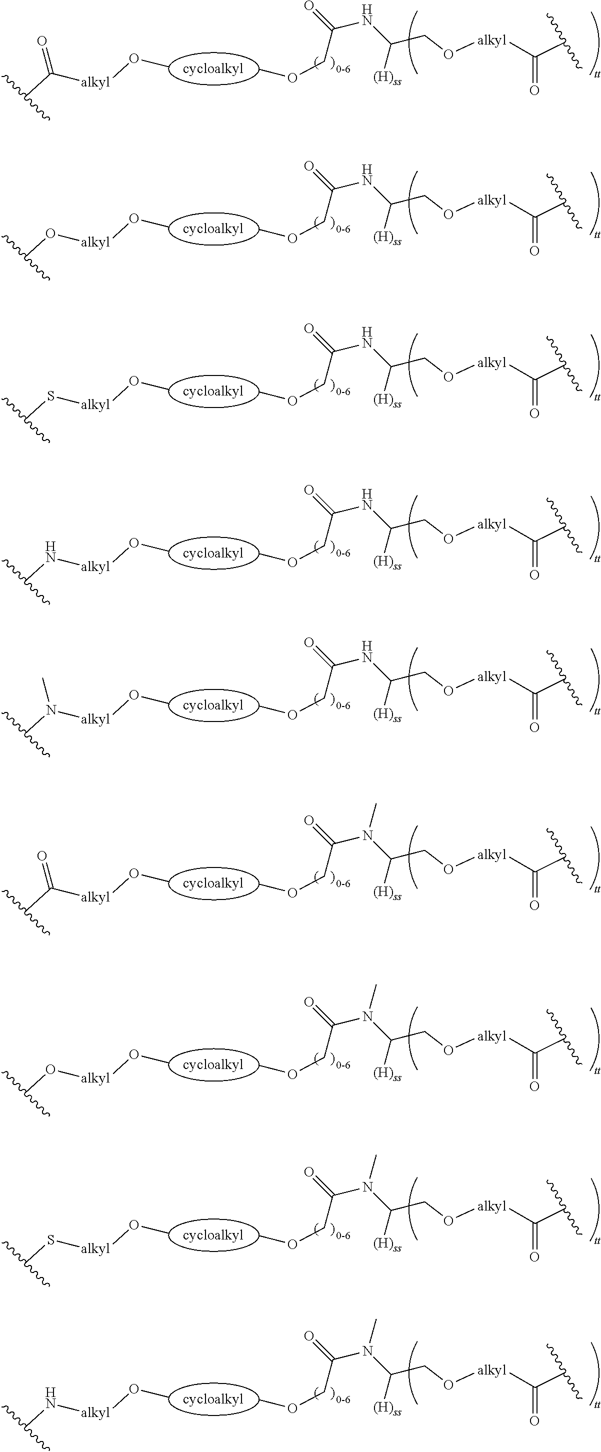

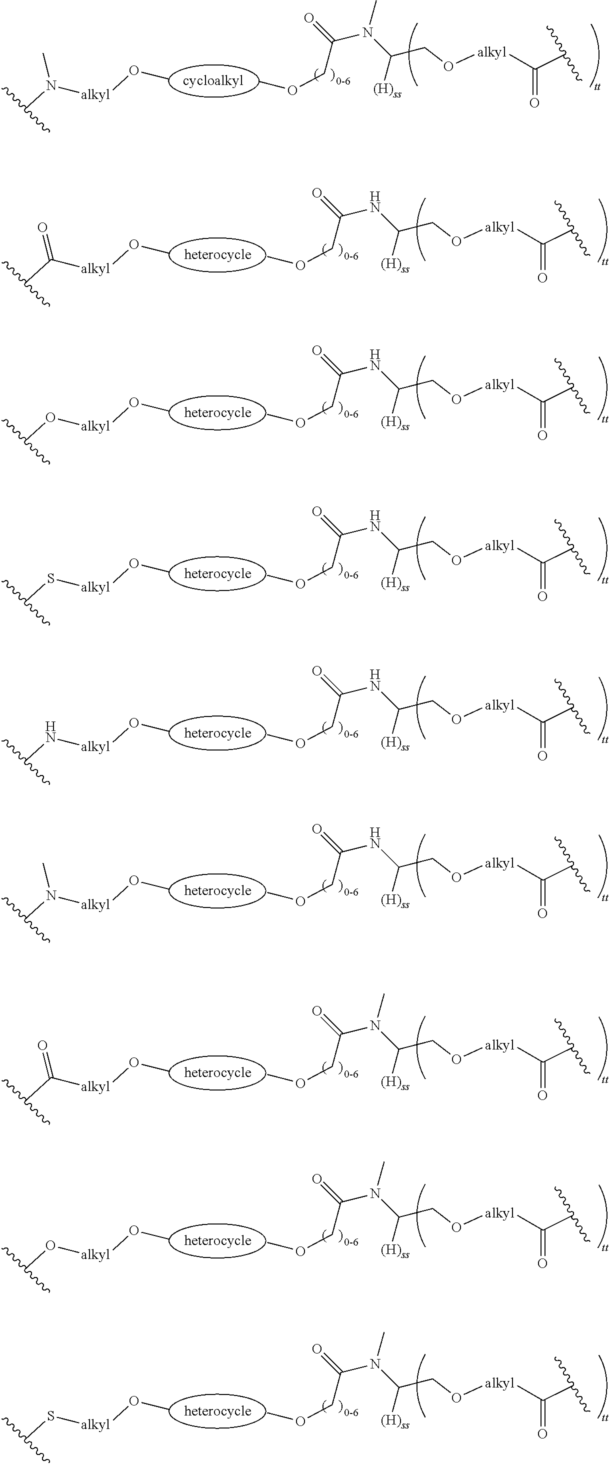

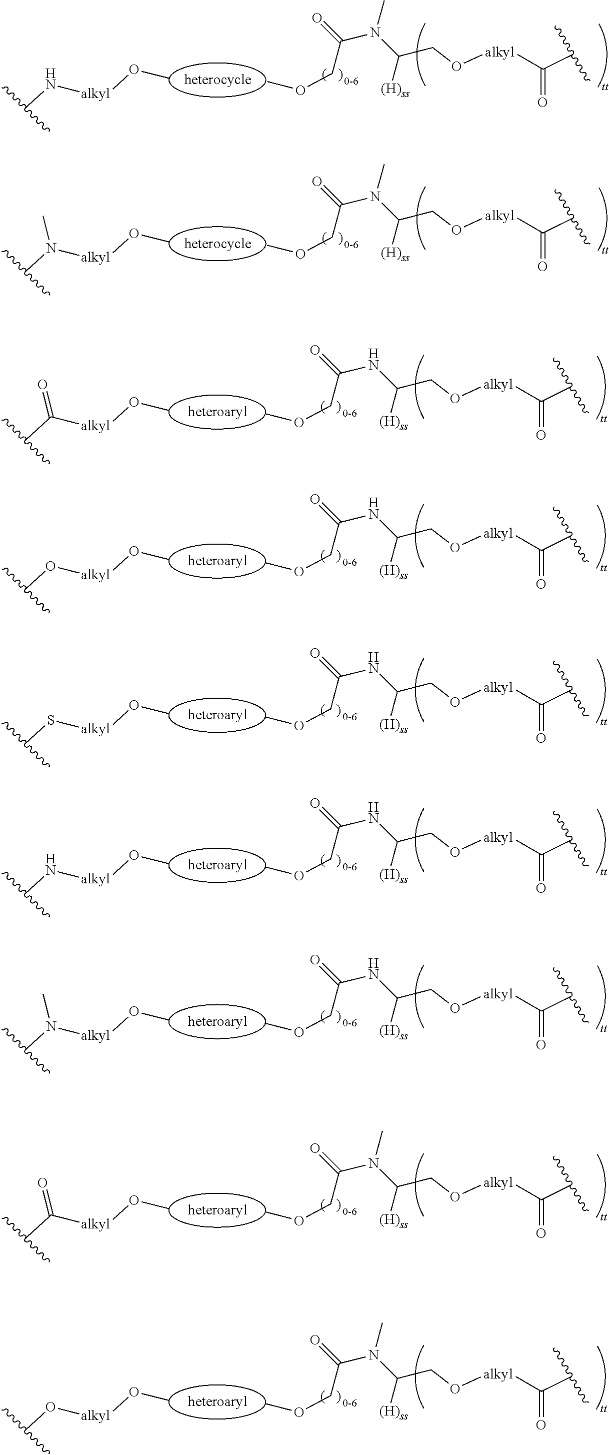

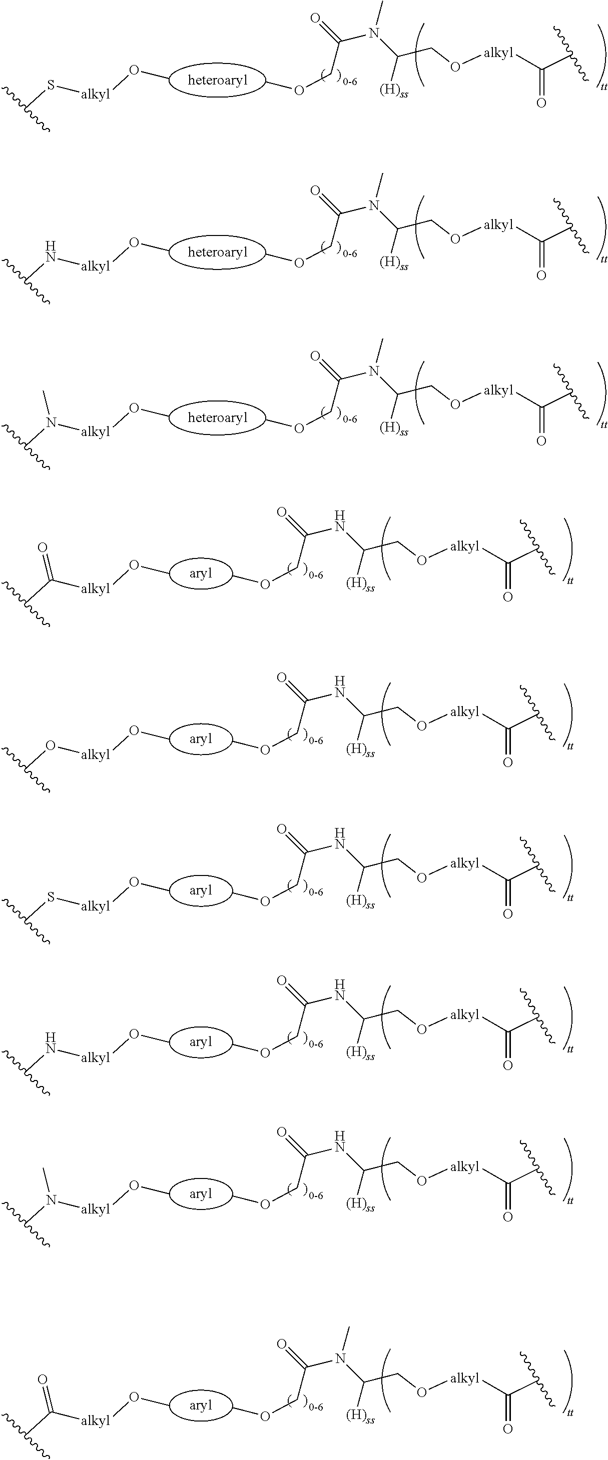

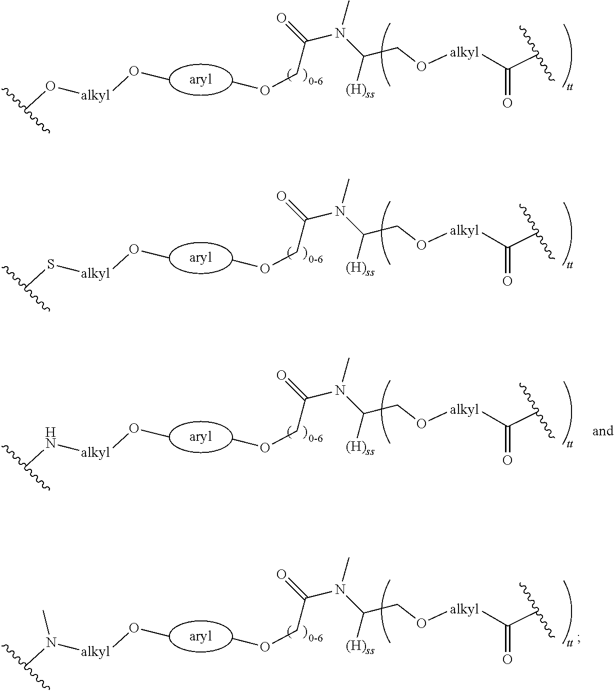

wherein each heteroaryl, heterocycle, cycloalkyl, and aryl can optionally be substituted with 1, 2, 3, or 4 of any combination of halogen, alkyl, haloalkyl, aryl, heteroaryl, heterocycle, or cycloalkyl, as allowed by valence; and tt and ss are as defined herein.

In certain embodiments Linker^{B}, Linker^{C}, or Linker^{D} is selected from:

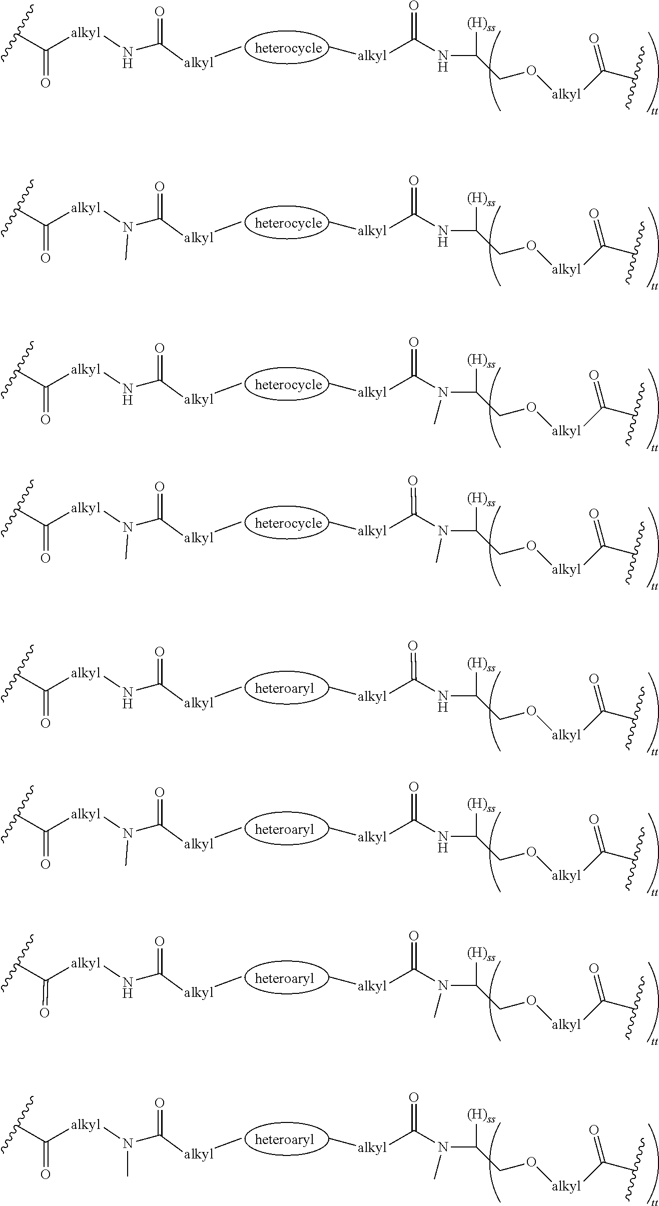

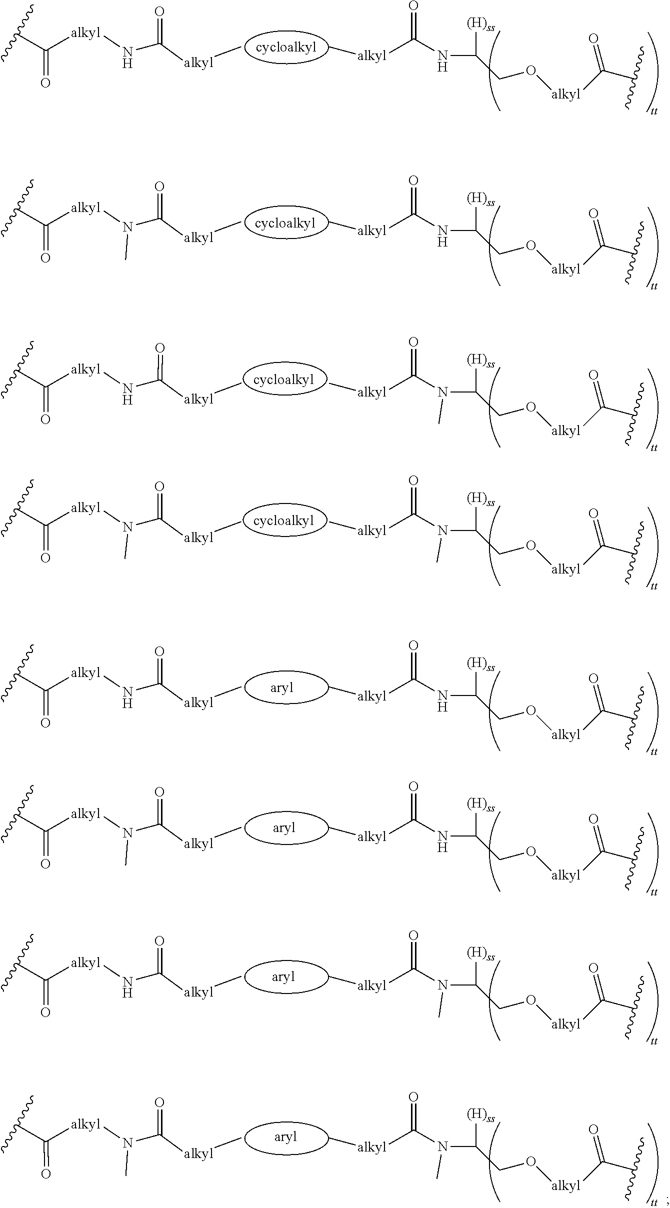

wherein each heteroaryl, heterocycle, cycloalkyl, and aryl can optionally be substituted with 1, 2, 3, or 4 of any combination of halogen, alkyl, haloalkyl, aryl, heteroaryl, heterocycle, or cycloalkyl, as allowed by valence; and tt and ss are as defined herein.

In certain embodiments Linker^{B}, Linker^{C}, or Linker^{D} is selected from:

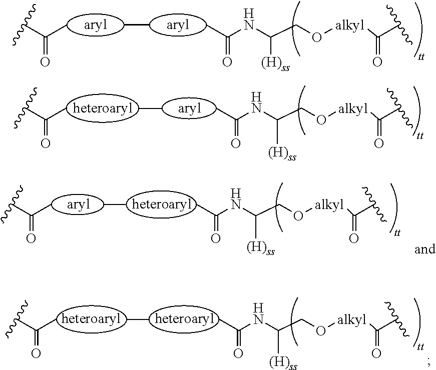

wherein each heteroaryl and aryl can optionally be substituted with 1, 2, 3, or 4 of any combination of halogen, alkyl, haloalkyl, aryl, heteroaryl, heterocycle, or cycloalkyl, as allowed by valence; and tt and ss are as defined herein.

In certain embodiments Linker^{B} is selected from:

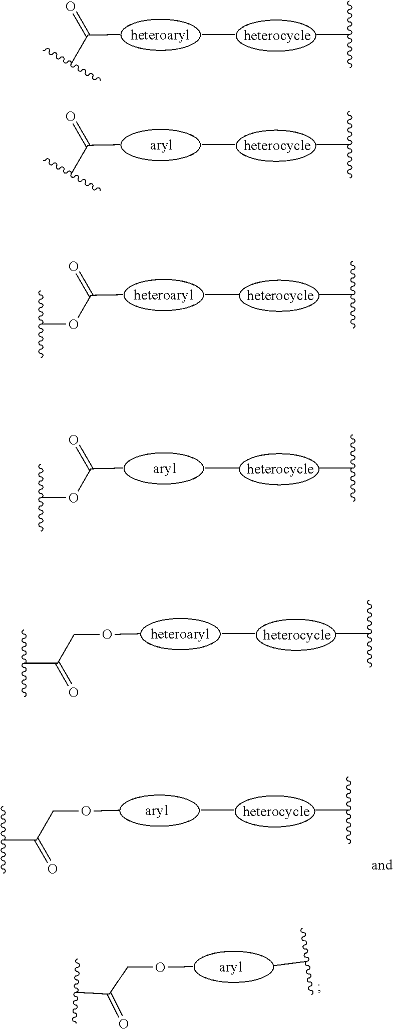

wherein each heteroaryl, heterocycle, cycloalkyl, and aryl can optionally be substituted with 1, 2, 3, or 4 of any combination of halogen, alkyl, haloalkyl, aryl, heteroaryl, heterocycle, or cycloalkyl, as allowed by valence.

In certain embodiments Linker® is selected from:

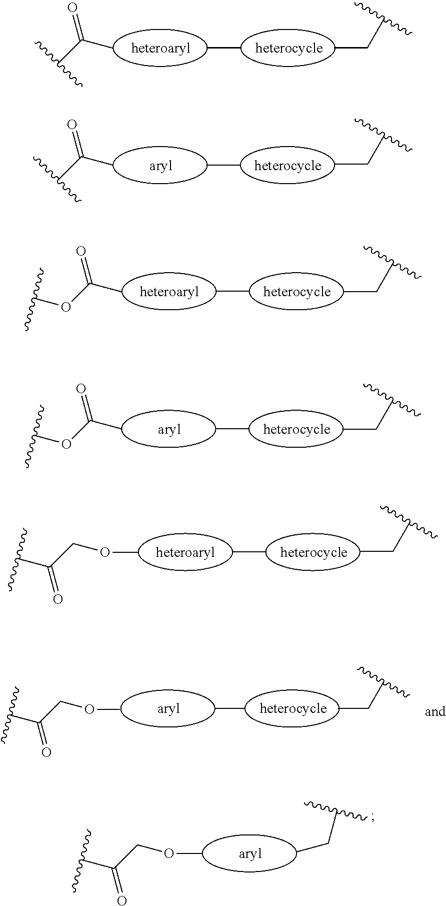

wherein each heteroaryl, heterocycle, cycloalkyl, and aryl can optionally be substituted with 1, 2, 3, or 4 of any combination of halogen, alkyl, haloalkyl, aryl, heteroaryl, heterocycle, or cycloalkyl, as allowed by valence.

In certain embodiments Linker^{B} is selected from:

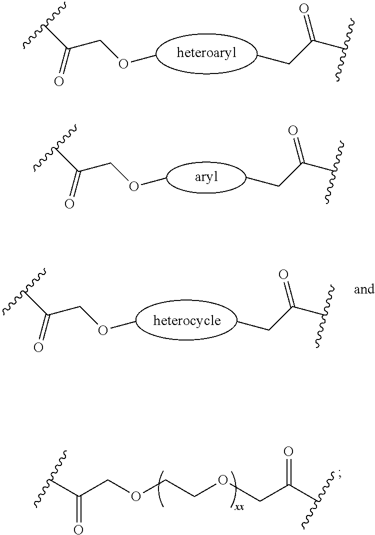

wherein each heteroaryl, heterocycle, cycloalkyl, and aryl can optionally be substituted with 1, 2, 3, or 4 of any combination of halogen, alkyl, haloalkyl, aryl, heteroaryl, heterocycle, or cycloalkyl, as allowed by valence.

In certain embodiments Linker^{B}, Linker^{C}, or Linker^{D} is selected from:

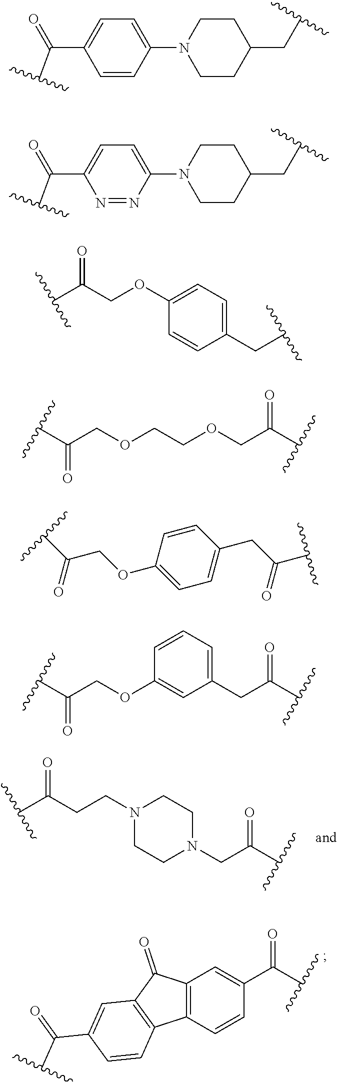

wherein each heteroaryl, heterocycle, cycloalkyl, and aryl can optionally be substituted with 1, 2, 3, or 4 of any combination of halogen, alkyl, haloalkyl, aryl, heteroaryl, heterocycle, or cycloalkyl, as allowed by valence.

In certain embodiments Linker^{A} is selected from:

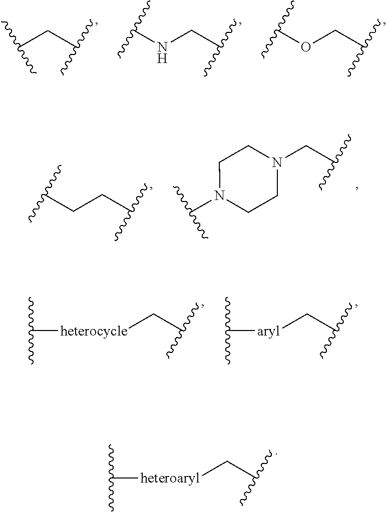

In certain embodiments Linker^{A} is selected from:

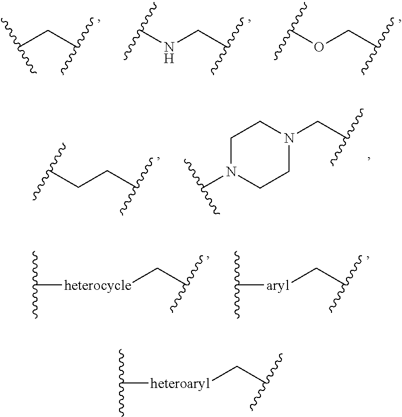

each of which is substituted with 1 or 2 optional substituents.

In certain embodiments Linker^{A} is bond.

In certain embodiments the left side of Linker^{A} is attached to the ASGPR Binding Ligand and the right side is attached to Linker^{B}, Linker^{C}, or Linker^{D}.

In certain embodiments the right side of Linker^{A} is attached to the ASGPR Binding Ligand and the right side is attached to Linker^{B}, Linker^{C}, or Linker^{D}.

In certain embodiments Linker^{B} is selected from:

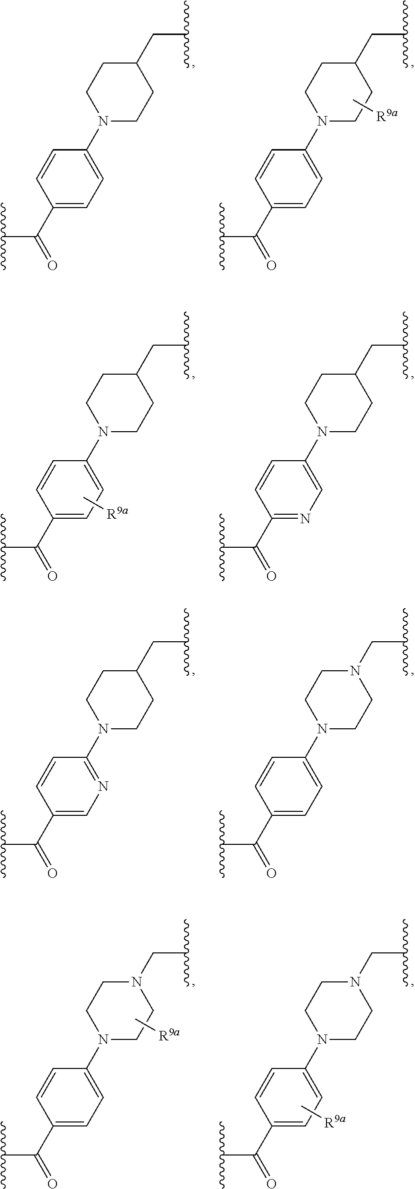

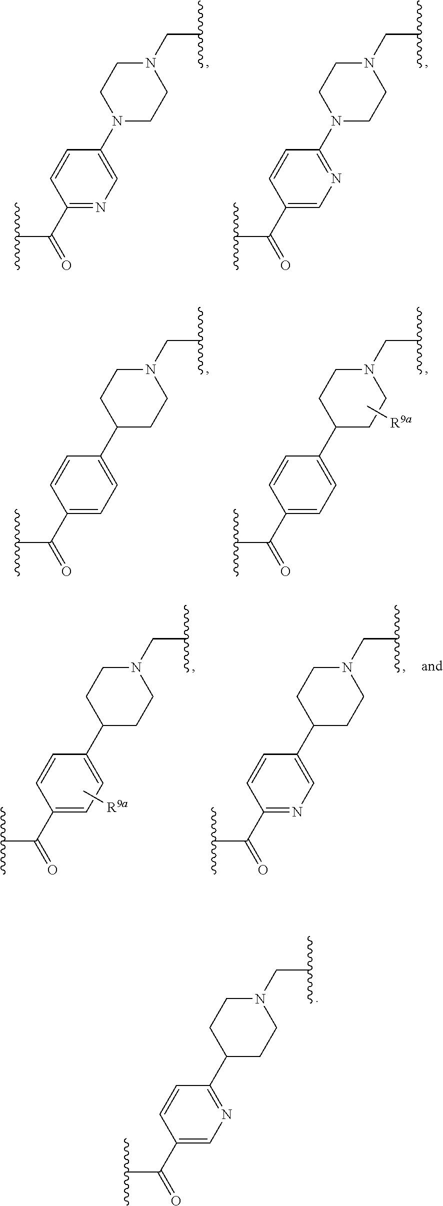

In certain embodiments Linker^{D} is selected from:

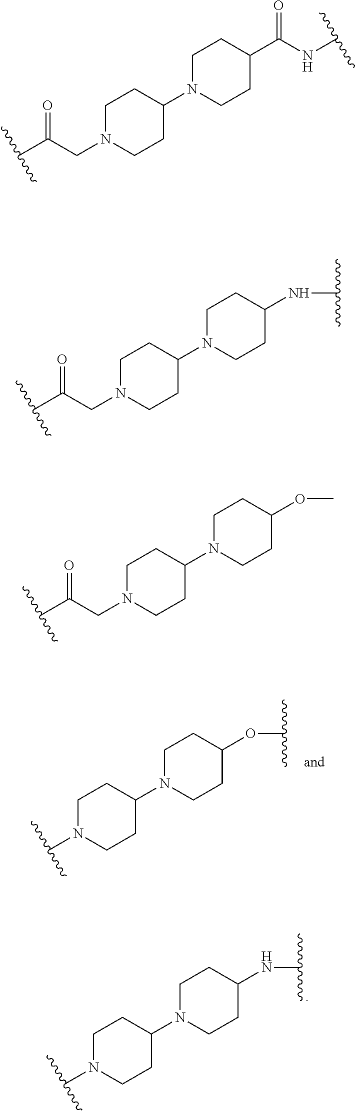 [SMILES: *NC(=O)C1CCN(C2CCN(CC(*)=O)CC2)CC1 ; *NC1CCN(C2CCN(CC(*)=O)CC2)CC1 ; *C(=O)CN1CCC(N2CCC(OC)CC2)CC1 ; *OC1CCN(C2CCN(*)CC2)CC1 ; *NC1CCN(C2CCN(*)CC2)CC1]

In certain embodiments Linker^{B} is selected from:

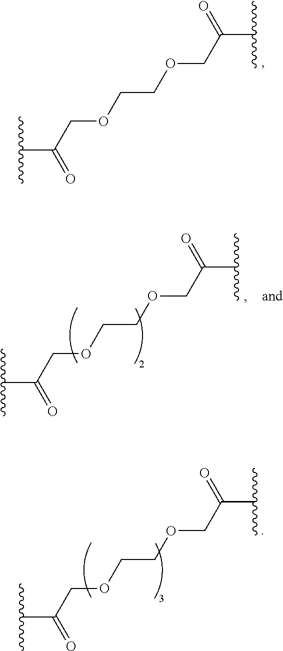

In certain embodiments the left side of Linker^{B} is attached to the Extracellular Targeting Ligand and the right side is attached to Linker^{A}.

In certain embodiments the right side of Linker^{B} is attached to the Extracellular Targeting Ligand and the left side is attached to Linker^{A}.

In certain embodiments Linker^{B} is bond.

In alternative embodiments a linker is provided as described above wherein

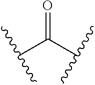 [SMILES: *C(*)=O]

is replaced with a

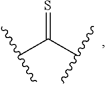 [SMILES: *C(*)=S]

for example where Linker^{B} is drawn as

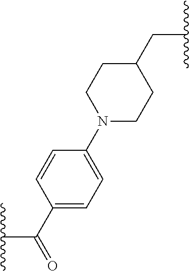 [SMILES: *CC1CCN(c2ccc(C(*)=O)cc2)CC1]

it is

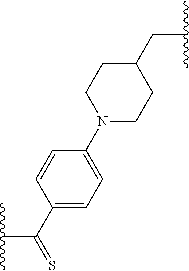 [SMILES: *CC1CCN(c2ccc(C(*)=S)cc2)CC1]

in this embodiment.

In alternative embodiments a linker is provided as described above wherein a

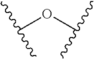 [SMILES: *O*]

is replaced with a

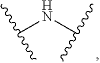 [SMILES: *N*]

for example where Linker^{B} is drawn as

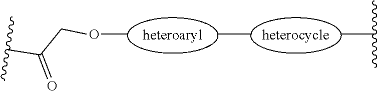

it is

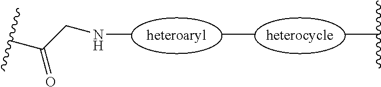

in this embodiment.

In alternative embodiments a linker is provided as described above wherein a

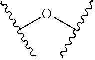 [SMILES: *O*]

is replaced with a

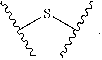 [SMILES: *S*]

In certain embodiments, R¹¹, R¹², R¹³, R¹⁴, R¹⁵, R¹⁶, R¹⁷, R¹⁸, R¹⁹, and R²⁰ are bond. In certain embodiments, R¹², R¹³, R¹⁴, R¹⁵, R¹⁶, R¹⁷, R¹⁸, R¹⁹, and R²⁰ are bond. In certain embodiments, R¹³, R¹⁴, R¹⁵, R¹⁶, R¹⁷, R¹⁸, R¹⁹, and R²⁰ are bond. In certain embodiments, R¹⁴, R¹⁵, R¹⁶, R¹⁷, R¹⁸, R¹⁹, and R²⁰ are bond. In certain embodiments, R¹⁵, R¹⁶, R¹⁷, R¹⁸, R¹⁹, and R²⁰ are bond. In certain embodiments, R¹⁶, R¹⁷, R¹⁸, R¹⁹, and R²⁰ are bond. In certain embodiments, R¹⁷, R¹⁸, R¹⁹, and R²⁰ are bond. In certain embodiments, R¹⁸, R¹⁹, and R²⁰ are bond.

In certain embodiments, nine of R¹¹, R¹², R¹³, R¹⁴, R¹⁵, R¹⁶, R¹⁷, R¹⁸, R¹⁹, and R²⁰ are bond. In certain embodiments, eight of R¹¹, R¹², R¹³, R¹⁴, R¹⁵, R¹⁶, R¹⁷, R¹⁸, R¹⁹, and R²⁰ are bond. In certain embodiments, seven of R¹¹, R¹², R¹³, R¹⁴, R¹⁵, R¹⁶, R¹⁷, R¹⁸, R¹⁹, and R²⁰ are bond. In certain embodiments, six of R¹¹, R¹², R¹³, R¹⁴, R¹⁵, R¹⁶, R¹⁷, R¹⁸, R¹⁹, and R²⁰ are bond. In certain embodiments, five of R¹¹, R¹², R¹³, R¹⁴, R¹⁵, R¹⁶, R¹⁷, R¹⁸, R¹⁹, and R²⁰ are bond. In certain embodiments, four of R¹¹, R¹², R¹³, R¹⁴, R¹⁵, R¹⁶, R¹⁷, R¹⁸, R¹⁹, and R²⁰ are bond. In certain embodiments, three of R¹¹, R¹², R¹³, R¹⁴, R¹⁵, R¹⁶, R¹⁷, R¹⁸, R¹⁹, and R²⁰ are bond. In certain embodiments, two of R¹¹, R¹², R¹³, R¹⁴, R¹⁵, R¹⁶, R¹⁷, R¹⁸, R¹⁹, and R²⁰ are bond. In certain embodiments, one of R¹¹, R¹², R¹³, R¹⁴, R¹⁵, R¹⁶, R¹⁷, R¹⁸, R¹⁹, and R²⁰ is bond.

In certain embodiments R¹¹ is attached to Linker^{A}.

In certain embodiments, Linker^{A} is

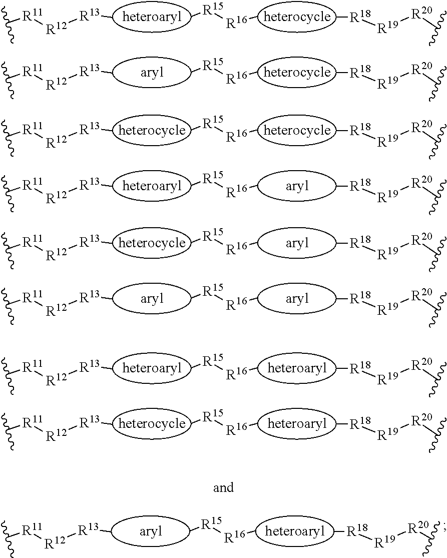

wherein each heteroaryl, heterocycle, and aryl can optionally be substituted with 1, 2, 3, or 4 of any combination of halogen, alkyl, haloalkyl, aryl, heteroaryl, heterocycle, or cycloalkyl.

In certain embodiments, R¹¹, R¹², R¹³, R¹⁵, R¹⁶, R¹⁸, R¹⁹, and R²⁰ are independently selected from bond, alkyl, —C(O)—, —C(O)O—, —OC(O)—, —C(O)NR⁶—, —NR^{9c}(O)—, —O—, —S—, —NR⁶-, —C(R²¹R²¹)—, alkenyl, alkynyl, haloalkyl, alkoxy, aryl, heterocycle, heteroaryl, and —CH₂CH₂—[O—(CH₂)₂]ₙ—O—.

In certain embodiments, Linker^{A} or Linker^{B} is selected from

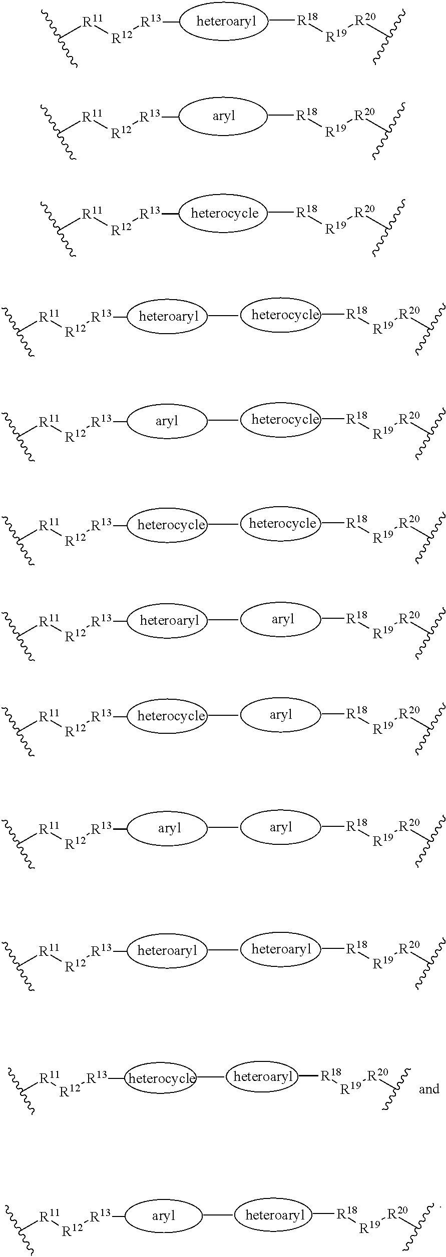

In certain embodiments, R¹¹, R¹², R¹³, R¹⁸, R¹⁹, and R²⁰ are independently selected from bond, alkyl, —C(O)—, —C(O)O—, —OC(O)—, —C(O)NR⁶—, —NR^{9c}(O)—, —O—, —S—, —NR⁶—, —C(R²¹R²¹)—, alkenyl, alkynyl, haloalkyl, alkoxy, aryl, heterocycle, heteroaryl, and —CH₂CH₂—[O—(CH₂)₂]ₙ—O—.

In certain embodiments, R¹¹ is selected from the group consisting of bond, CH₂, —O—, —C(O)NR⁶- and —C(O)O—. In certain embodiments, R²⁰ is selected from the group consisting of bond, CH₂, —O—, —C(O)NR⁶- and —C(O)O—.

In certain embodiments, Linker^{A} or Linker^{B} is selected from

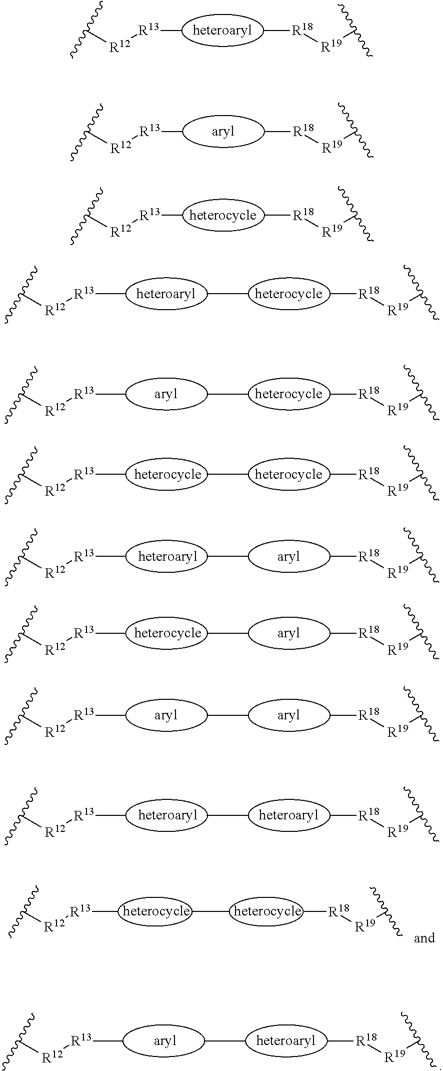

In certain embodiments, R¹² is selected from the group consisting of bond, CH₂, —O—, —C(O)NR⁶- and —C(O)O—. In certain embodiments, R¹⁹ is selected from the group consisting of bond, CH₂, —O—, —C(O)NR⁶- and —C(O)O-.

In certain embodiments, Linker or Linker® is selected from

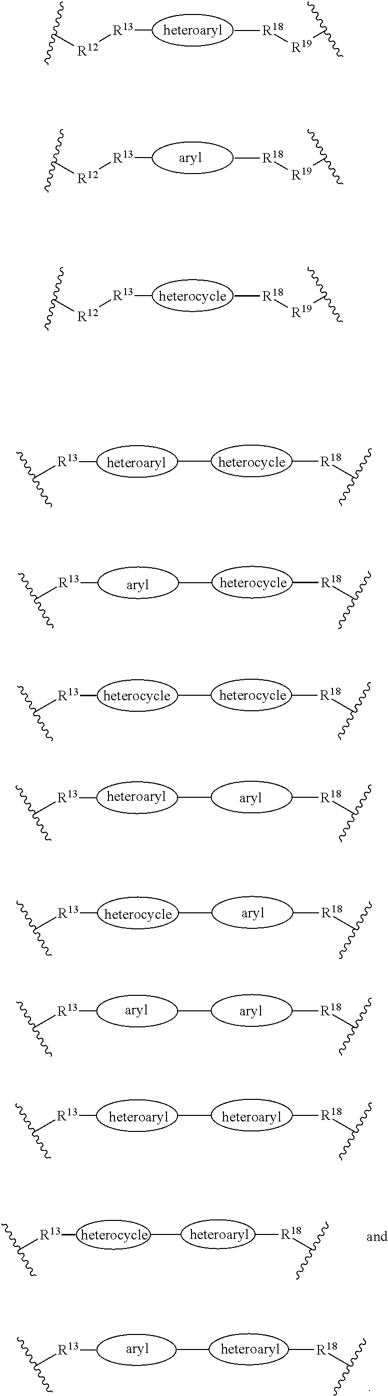

In certain embodiments, R¹³ is selected from the group consisting of bond, CH₂, —O—, —C(O)NR⁶- and —C(O)O—. In certain embodiments, R¹⁸ is selected from the group consisting of bond, CH₂, —O—, —C(O)NR⁶- and —C(O)O—.

In certain embodiments, aryl is phenyl.

In certain embodiments, heteroaryl is selected from

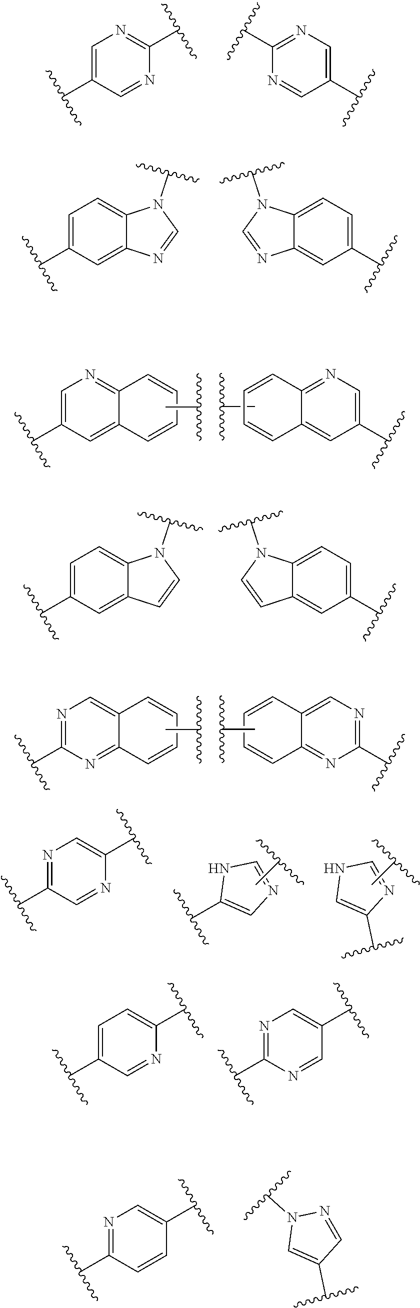

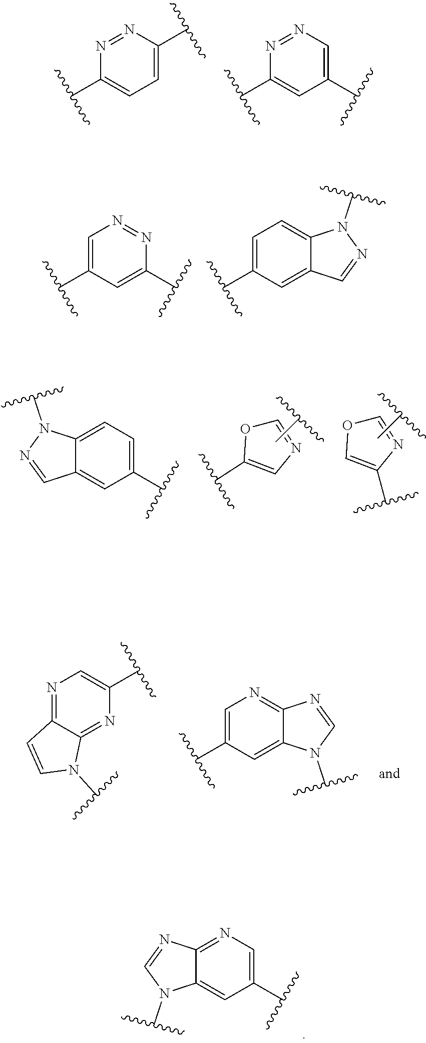

In certain embodiments, heteroaryl is selected from

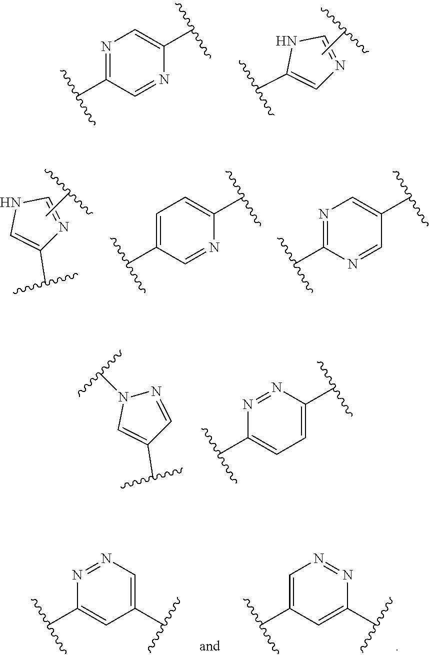

In certain embodiments, heterocycle is selected from

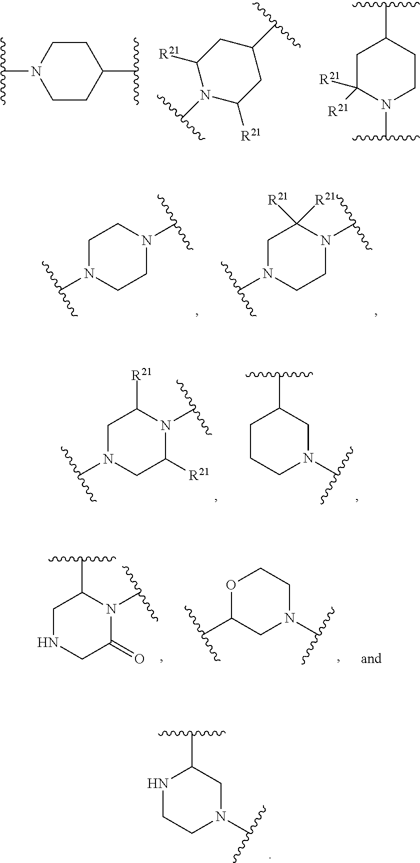

In certain embodiments, Linker^{A} or Linker^{B} is selected from

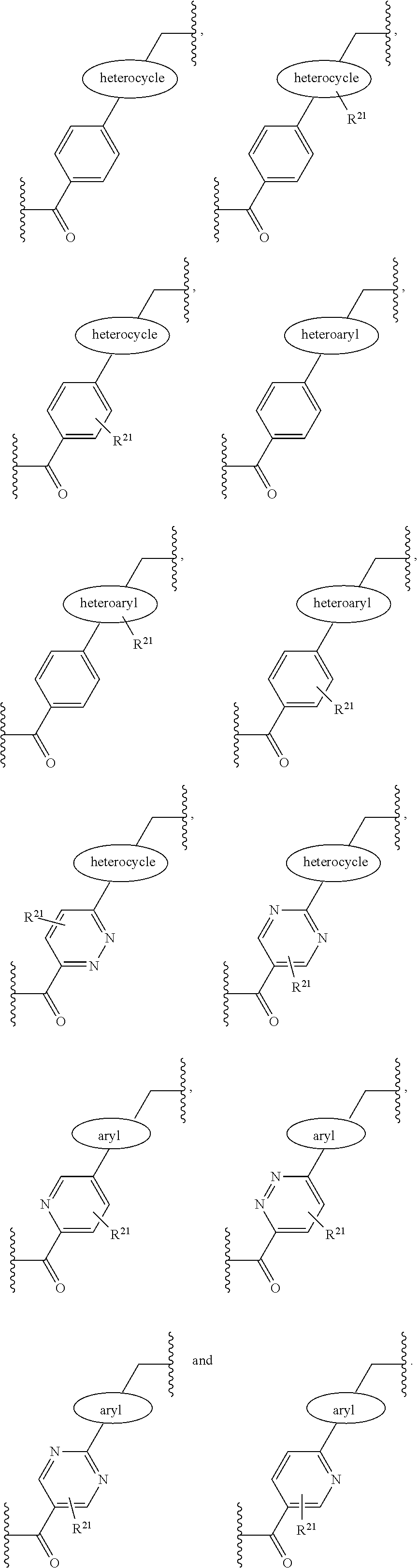

In certain embodiments, Linker^{A} or Linker^{B} is selected from

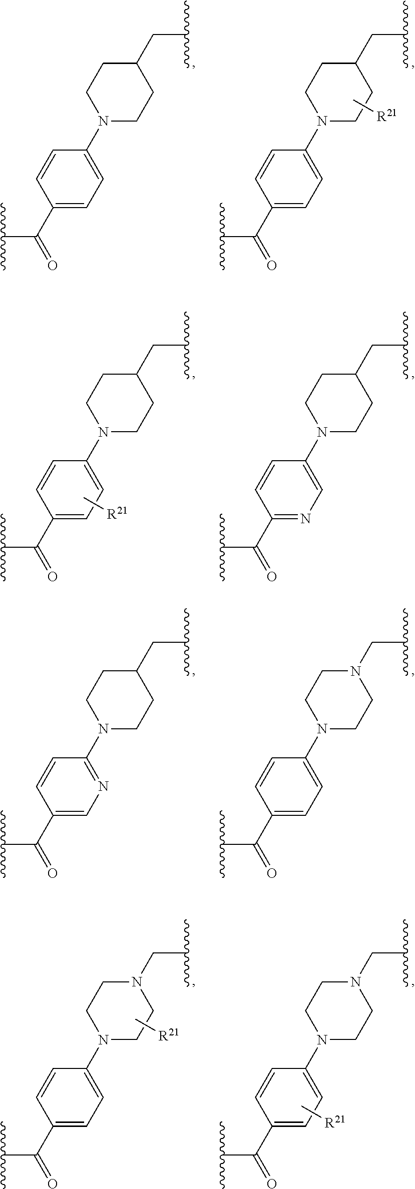

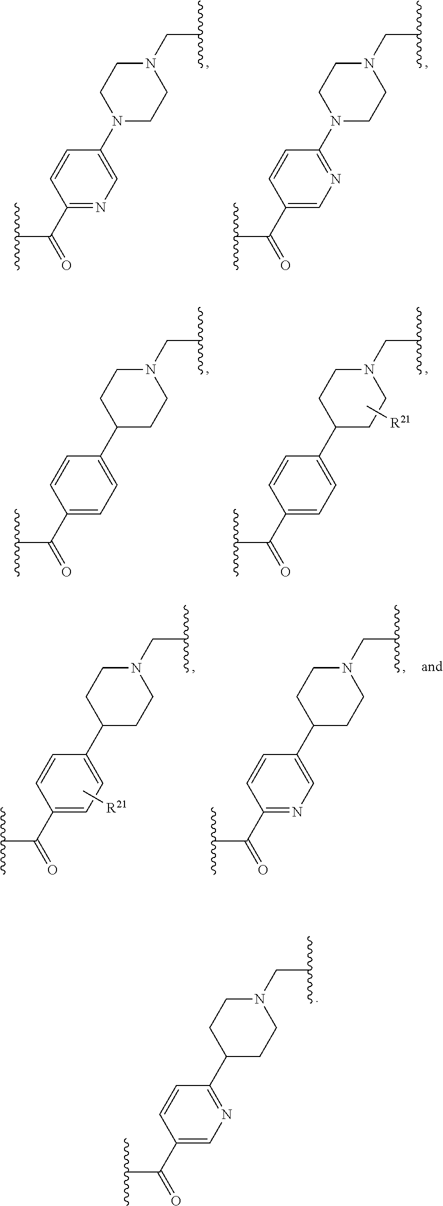

In certain embodiments, Linker^{B} is:

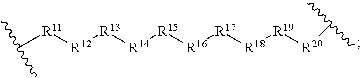

wherein:
R¹¹, R¹², R¹³, R¹⁴, R¹⁵, R¹⁶, R¹⁷, R¹⁸, R¹⁹, and R²⁰ are independently at each occurrence selected from the group consisting of a bond, alkyl, —C(O)—, —C(O)O—, —OC(O)—, —SO₂—, —S(O)—, —C(S)—, —C(O)NR⁶—, —NR^{9c}(O)—, —O—, —S—, —NR⁶—, —C(R²¹R²¹)—, —P(O)(R³)O—, —P(O)(R³)—, alkenyl, alkynyl, haloalkyl, alkoxy, aryl, heterocycle, heteroaryl, —CH₂CH₂—[O—(CH₂)₂]ₙ—O—, and —CH₂CH₂—[O—(CH₂)₂]ₙ—NR⁶—; each of which is optionally substituted with 1, 2, 3, or 4 substituents independently selected from R²¹;
n is independently selected at each instance from 0, 1, 2, 3, 4, 5, 6, 7, 8, 9, or 10;
R²¹ is independently at each occurrence selected from the group consisting of hydrogen, alkyl, alkenyl, alkynyl, F, Cl, Br, I, hydroxyl, alkoxy, azide, amino, cyano, —NR⁶R⁷, —NR⁸SO₂R³, —NR⁸S(O)R³, haloalkyl, aryl, heteroaryl, and heterocycle.

In certain embodiments of Linker^{B}, R¹¹, R¹², R¹³, R¹⁴, R¹⁵, R¹⁶, and R¹⁷ are bond. In certain embodiments of Linker^{B}, five of R¹¹, R¹², R¹³, R¹⁴, R¹⁵, R¹⁶, R¹⁷, R¹⁸, and R¹⁹ are bond. In certain embodiments of Linker^{B}, four of R¹¹, R¹², R¹³, R¹⁴, R¹⁵, R¹⁶, R¹⁷, R¹⁸, and R¹⁹ are bond. In certain embodiments of Linker^{B}, three of R¹¹, R¹², R¹³, R¹⁴, R¹⁵, R¹⁶, R¹⁷, R¹⁸, and R¹⁹ are bond. In certain embodiments of Linker^{B}, R¹⁸, R¹⁹, and R²⁰ are independently selected from bond, alkyl, —C(O)—, —C(O)O—, —OC(O)—, —C(O)NR⁶—, —NR⁶C(O)—, —O—, —S—, —NR⁶—, —C(R²¹R²¹)—, alkenyl, alkynyl, haloalkyl, alkoxy, aryl, heterocycle, heteroaryl, and —CH₂CH₂—[O—(CH₂)₂]ₙ—O—.

In certain embodiments Linker^{B} is selected from:

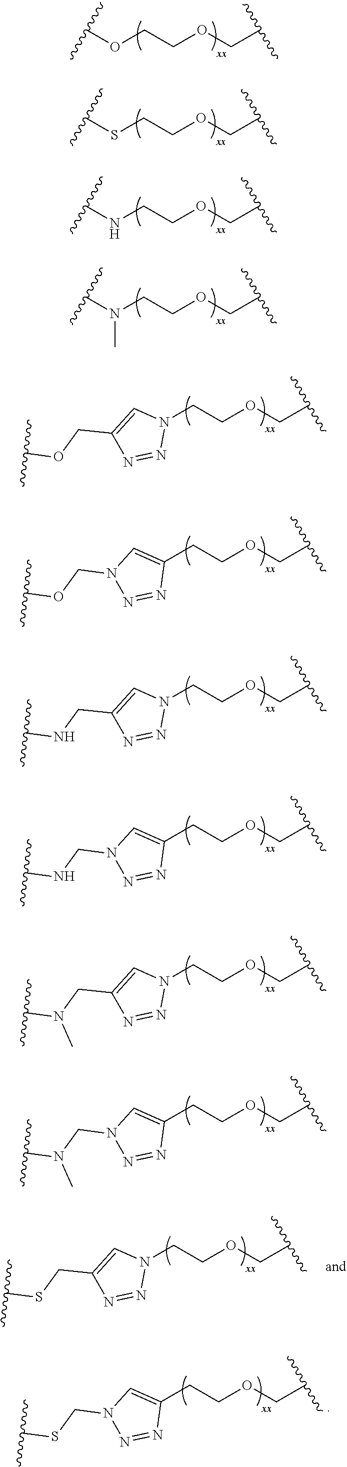

In certain embodiments Linker^{A} is selected from:

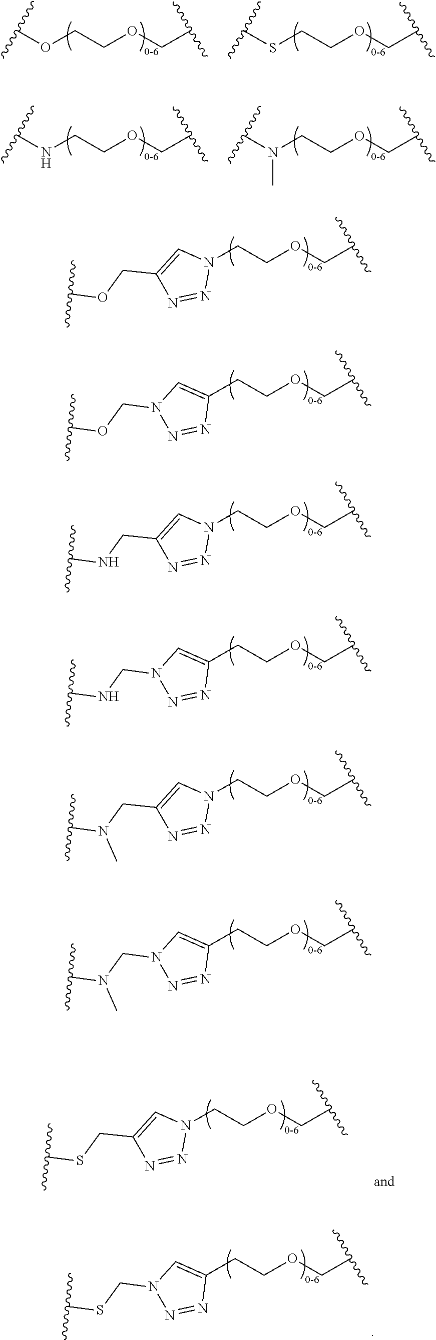

In certain embodiments Linker^{A} is selected from:

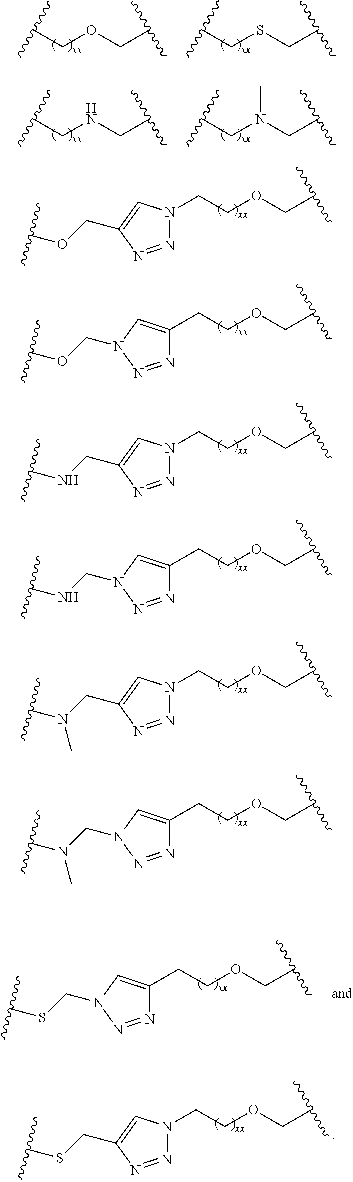

In certain embodiments Linker^{A} is selected from:

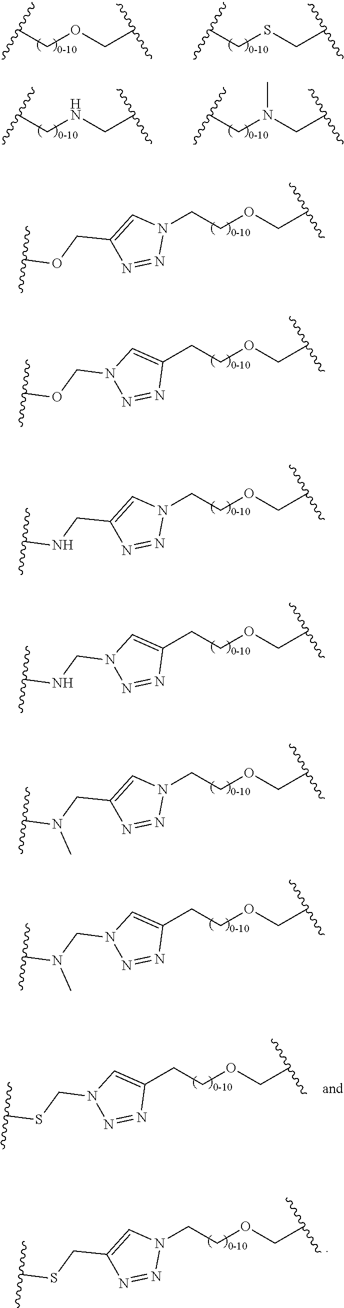

In certain embodiments Linker^{B} is selected from:

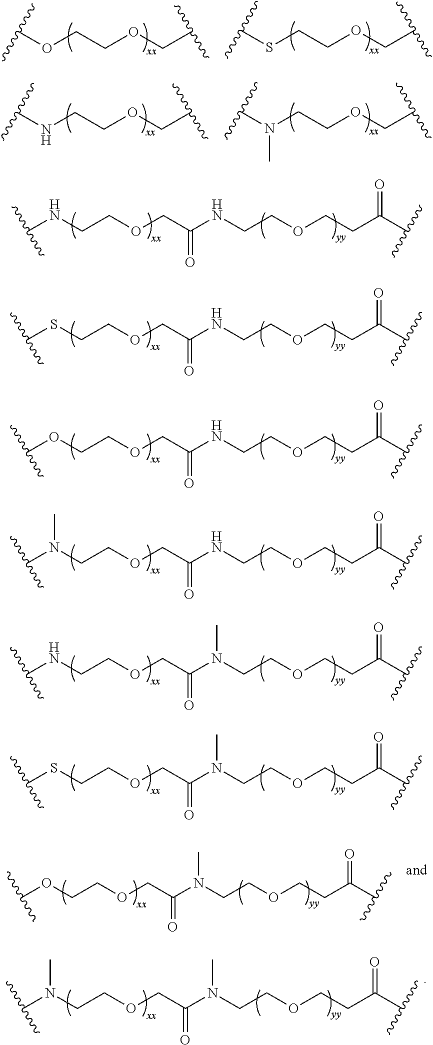

In certain embodiments Linker^{B} is selected from:

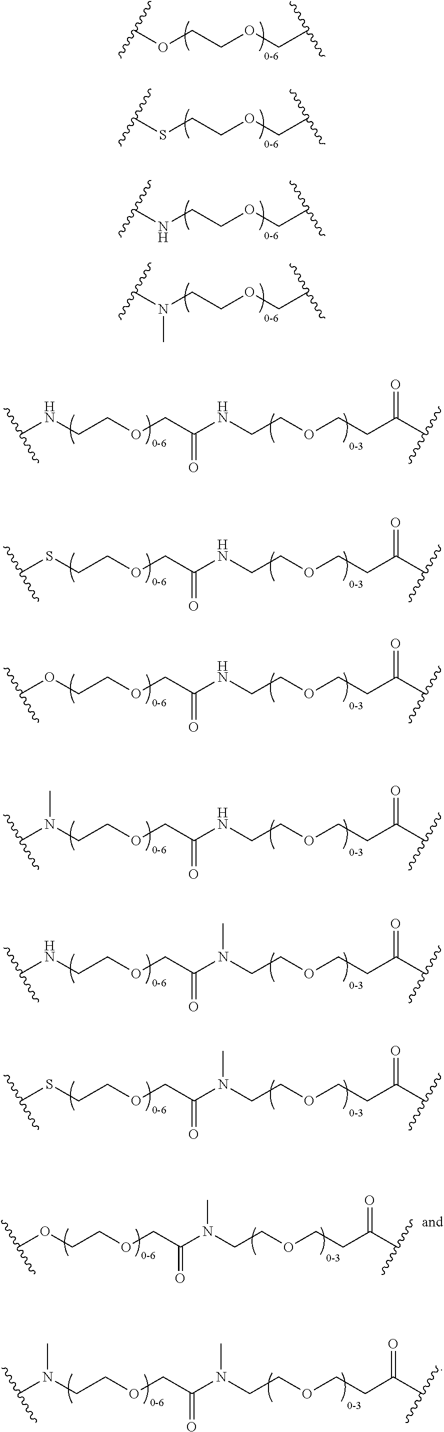

In certain embodiments Linker^{B} is selected from:

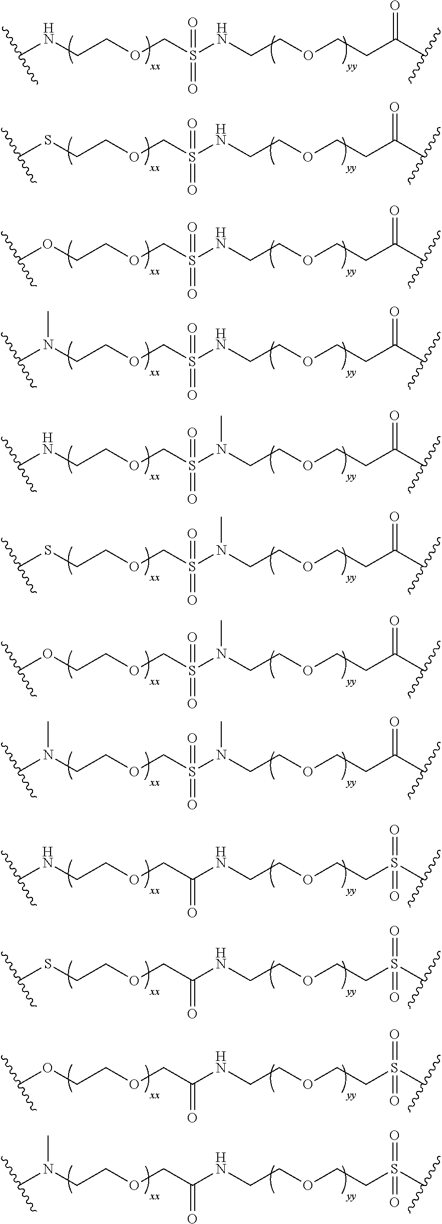

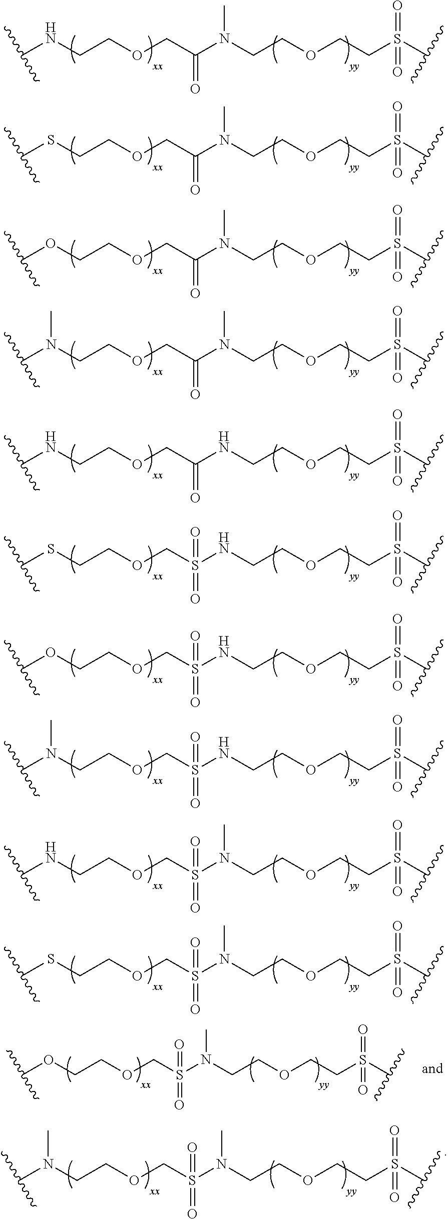

In certain embodiments Linker^{D} is selected from:

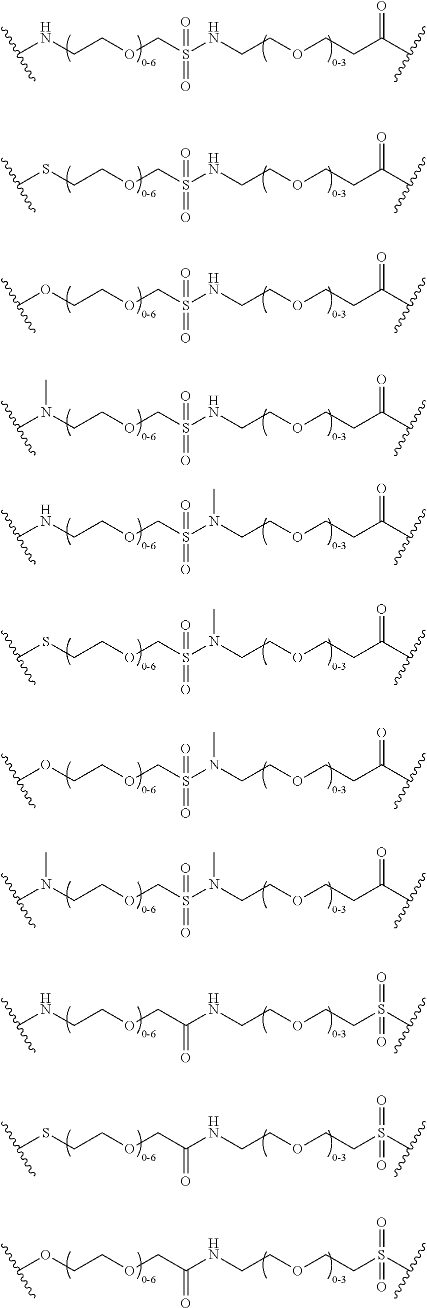

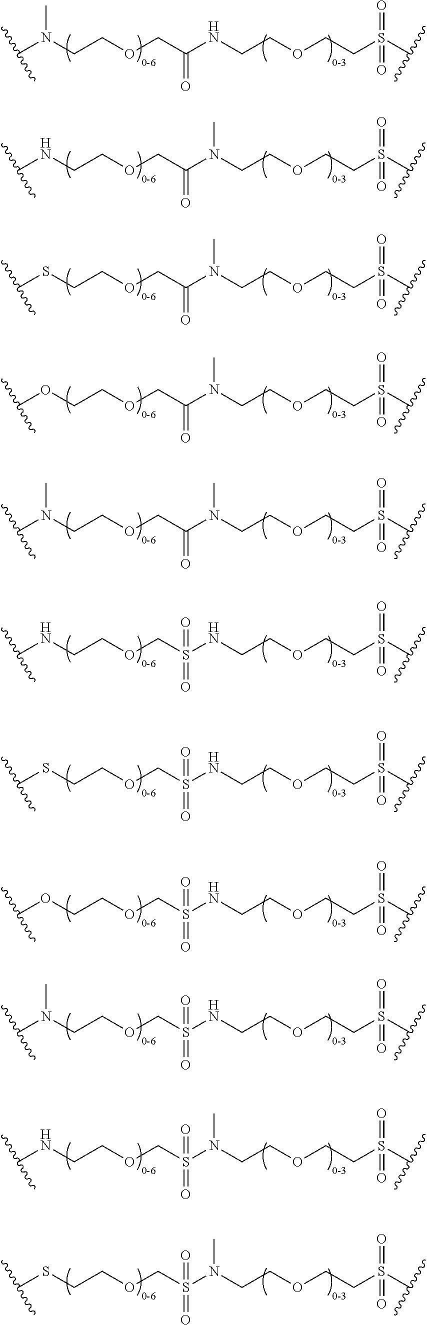

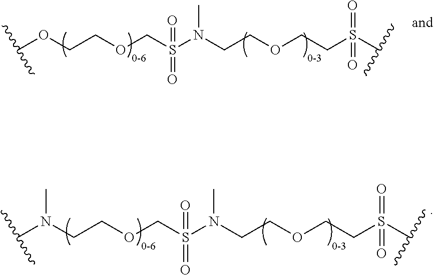

In certain embodiments Linker^{D} is selected from:

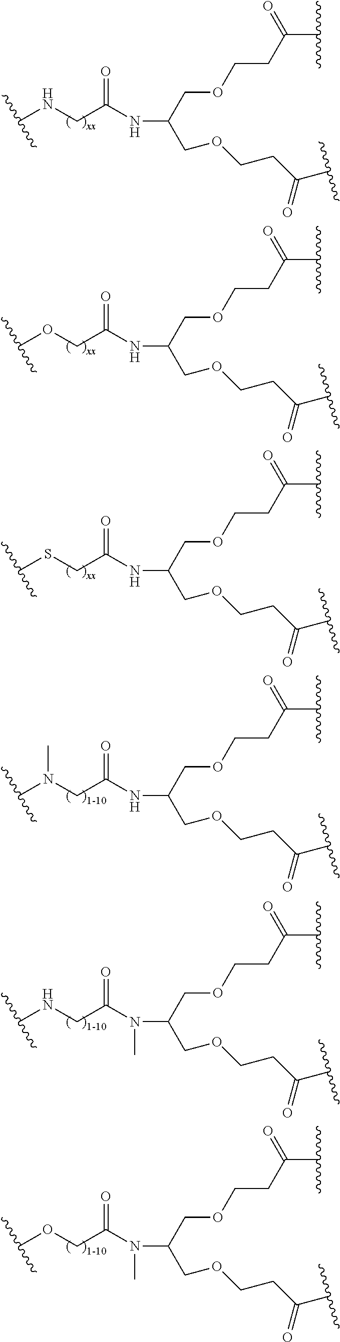

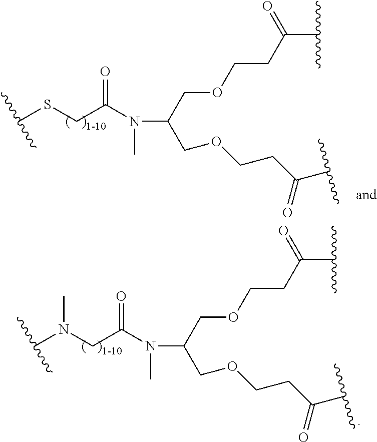

In certain embodiments Linker^{D} is selected from:

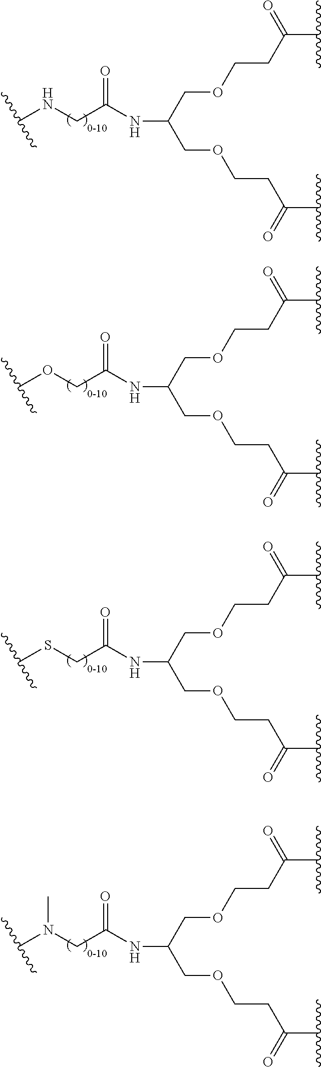

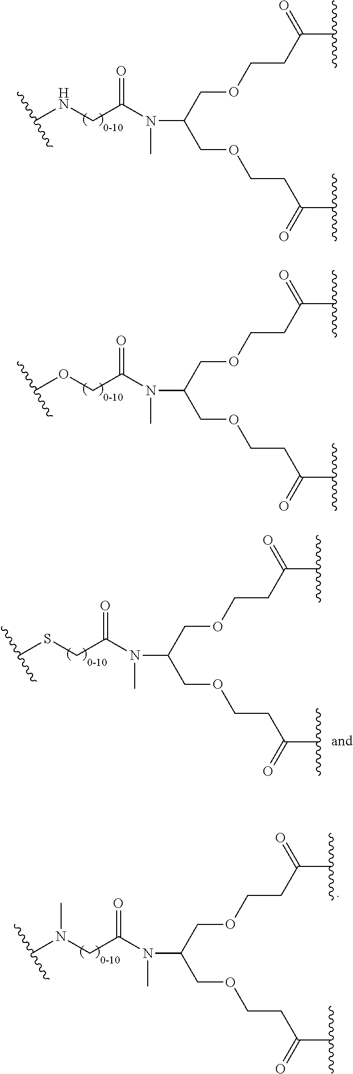

In certain embodiments Linker^{D} is selected from:

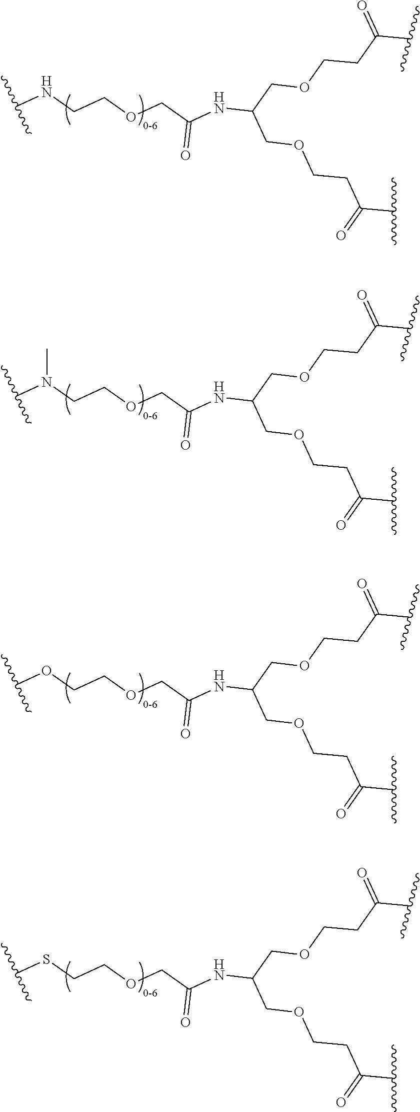

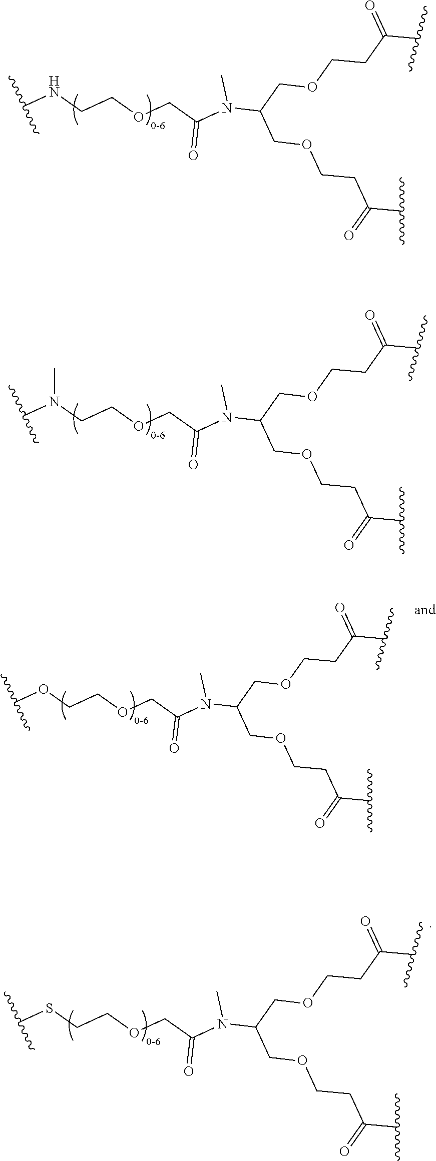

In certain embodiments Linker^{D} is selected from:

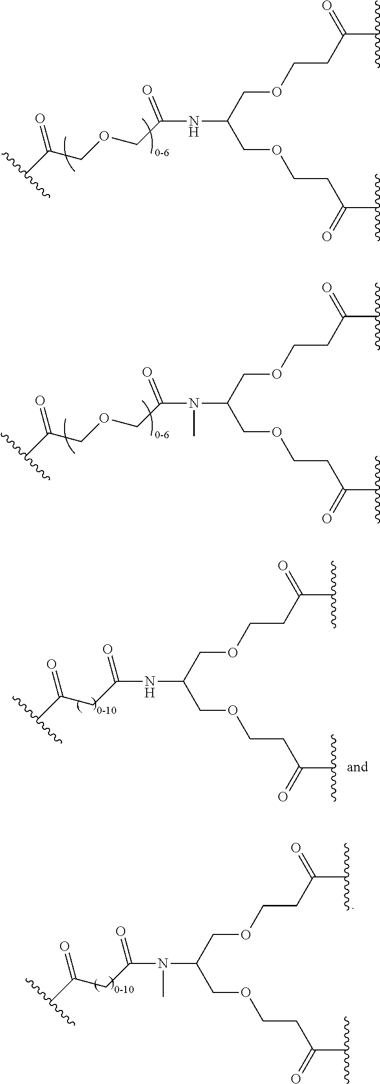

In certain embodiments Linker^{D} is selected from:

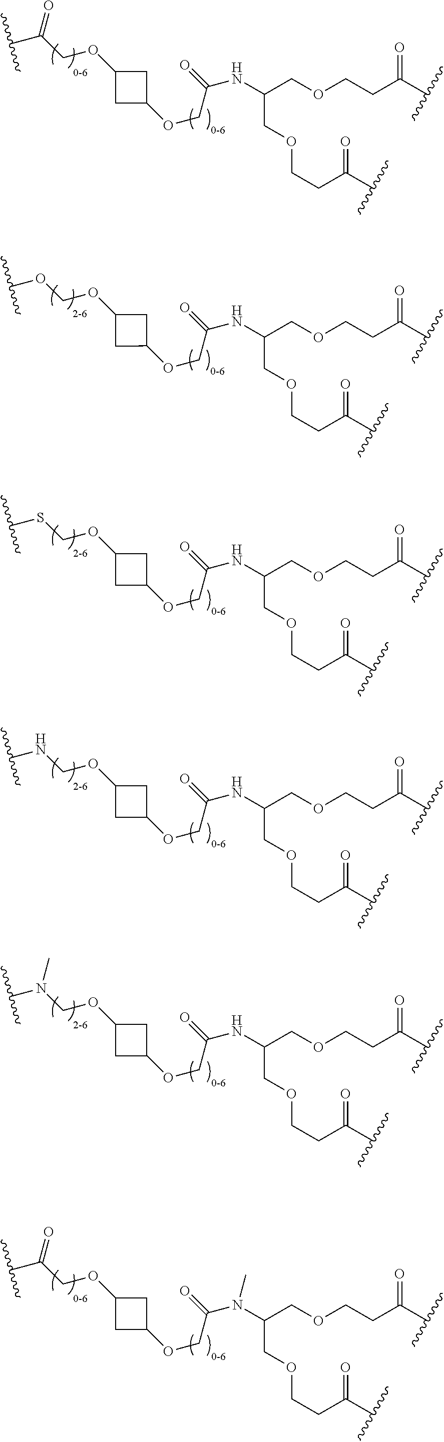

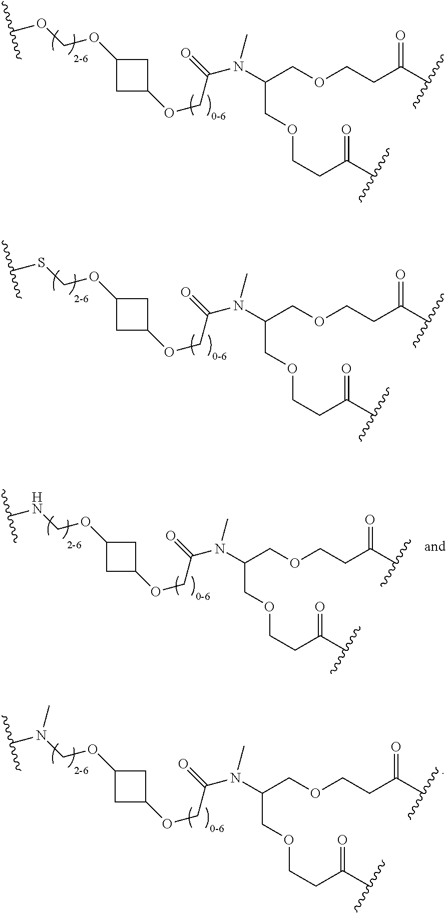

In certain embodiments Linker^{D} is selected from:

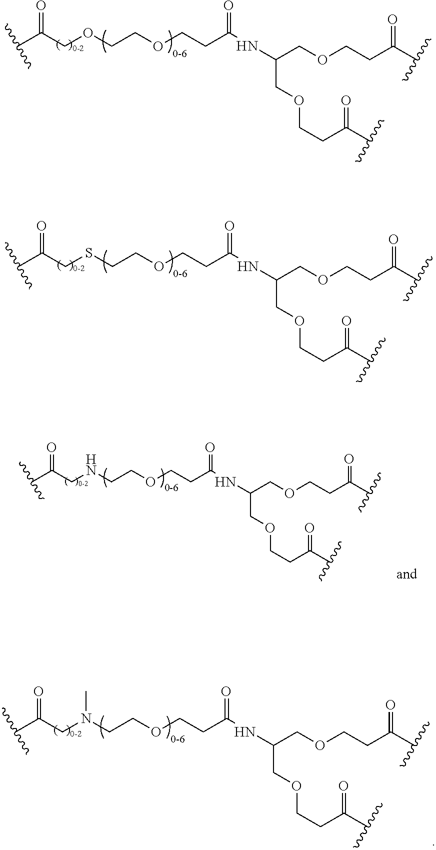

In certain embodiments Linker^{D} is selected from:

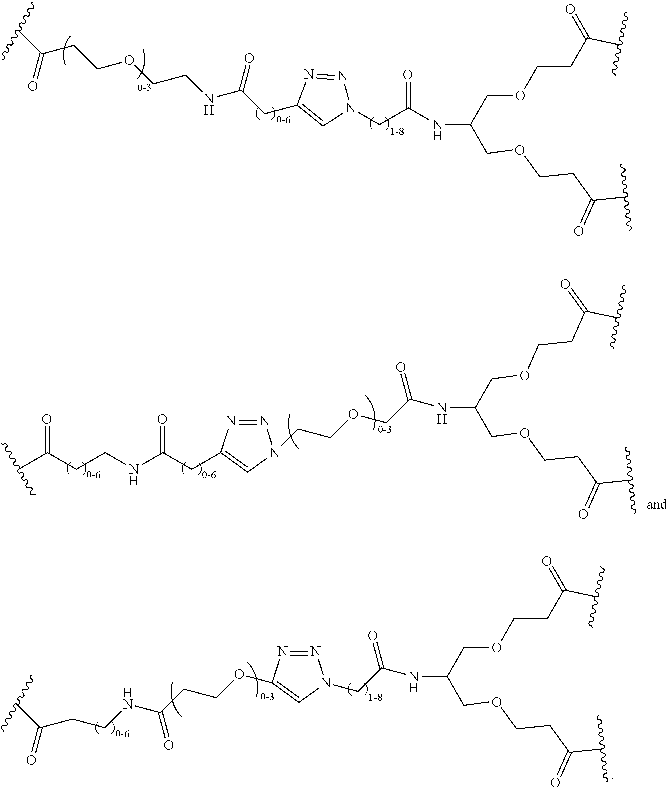

In certain embodiments Linker^{C} is selected from:

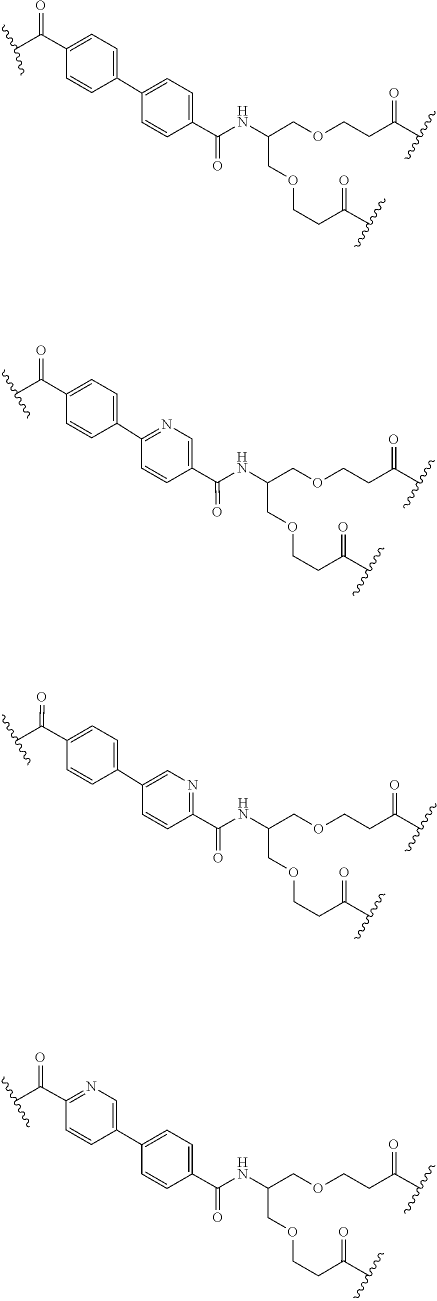 [SMILES: *C(=O)CCOCC(COCCC(*)=O)NC(=O)c1ccc(-c2ccc(C(*)=O)cc2)cc1 ; *C(=O)CCOCC(COCCC(*)=O)NC(=O)c1ccc(-c2ccc(C(*)=O)cc2)nc1 ; *C(=O)CCOCC(COCCC(*)=O)NC(=O)c1ccc(-c2ccc(C(*)=O)cc2)cn1 ; *C(=O)CCOCC(COCCC(*)=O)NC(=O)c1ccc(-c2ccc(C(*)=O)nc2)cc1]

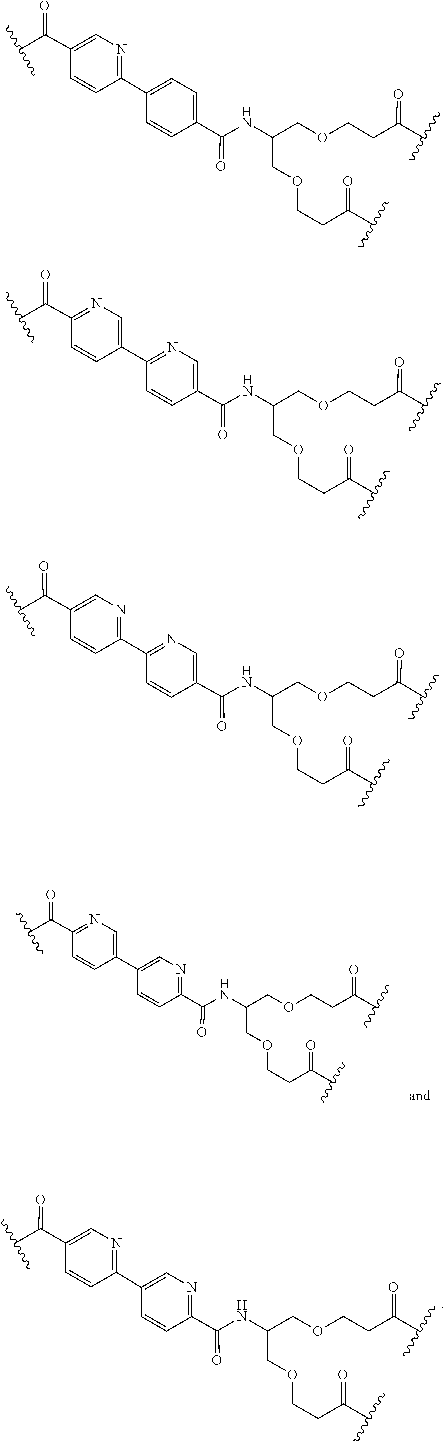 [SMILES: *C(=O)CCOCC(COCCC(*)=O)NC(=O)c1ccc(-c2ccc(C(*)=O)cn2)cc1 ; *C(=O)CCOCC(COCCC(*)=O)NC(=O)c1ccc(-c2ccc(C(*)=O)nc2)nc1 ; *C(=O)CCOCC(COCCC(*)=O)NC(=O)c1ccc(-c2ccc(C(*)=O)cn2)nc1 ; *C(=O)CCOCC(COCCC(*)=O)NC(=O)c1ccc(-c2ccc(C(*)=O)nc2)cn1 ; *C(=O)CCOCC(COCCC(*)=O)NC(=O)c1ccc(-c2ccc(C(*)=O)cn2)cn1]

In certain embodiments Linker^{D} is selected from:

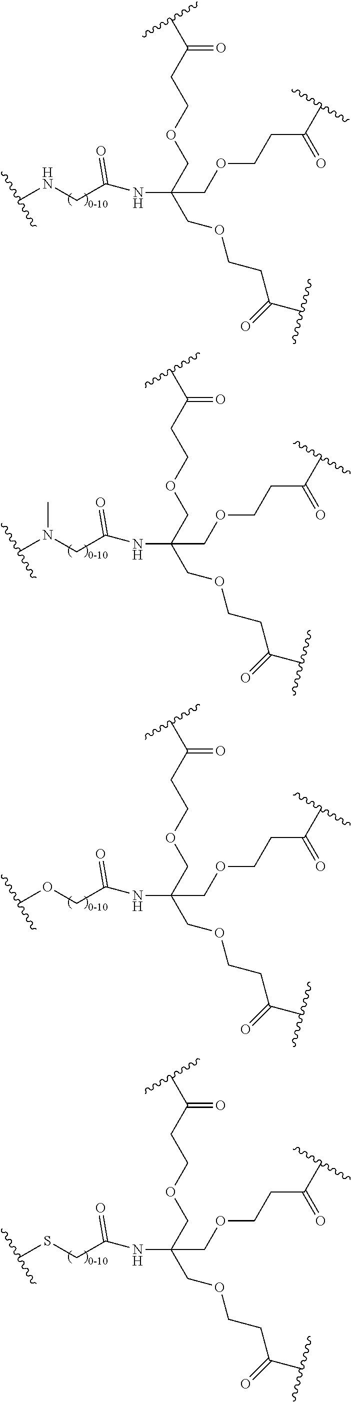

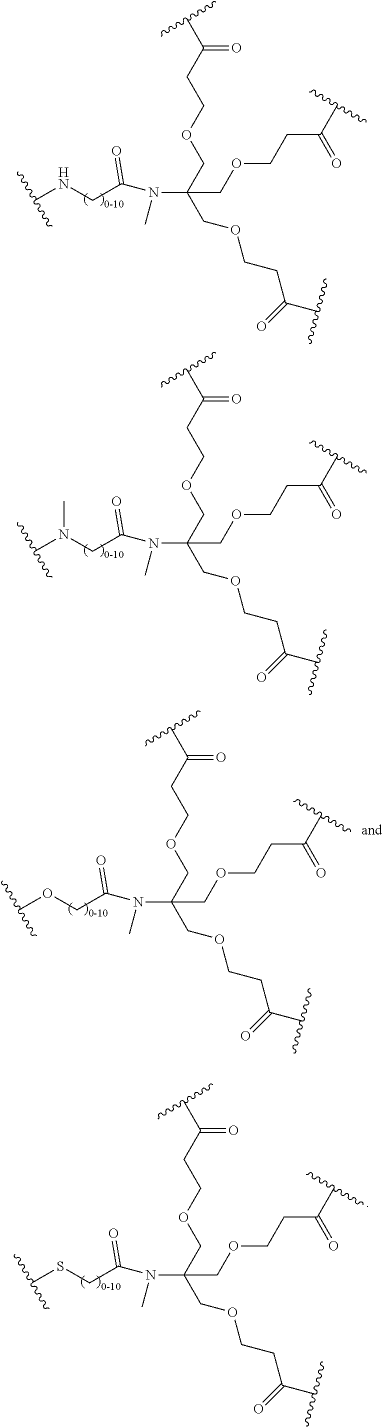

In certain embodiments Linker^{D} is selected from:

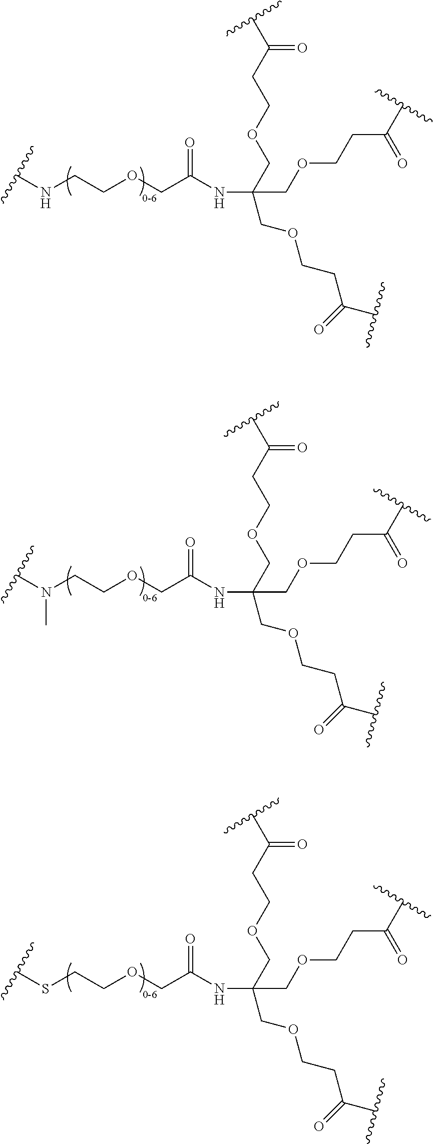

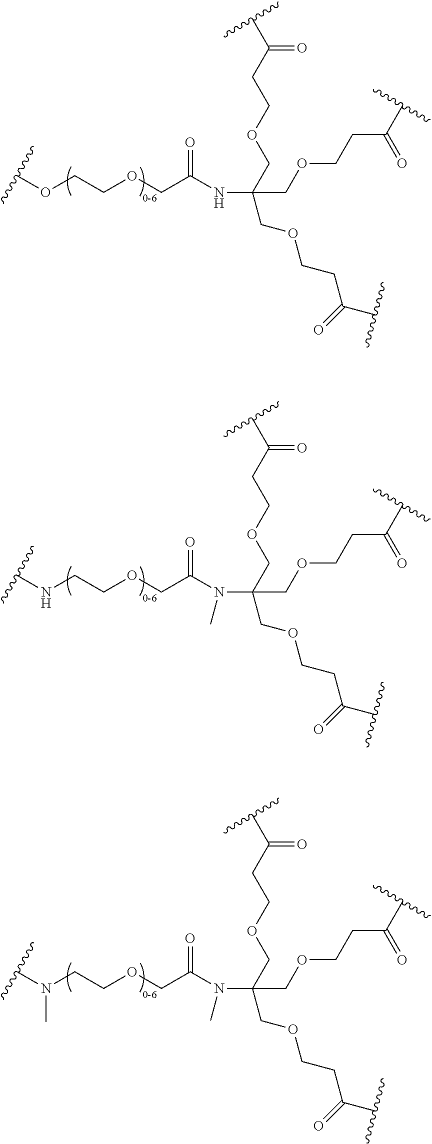

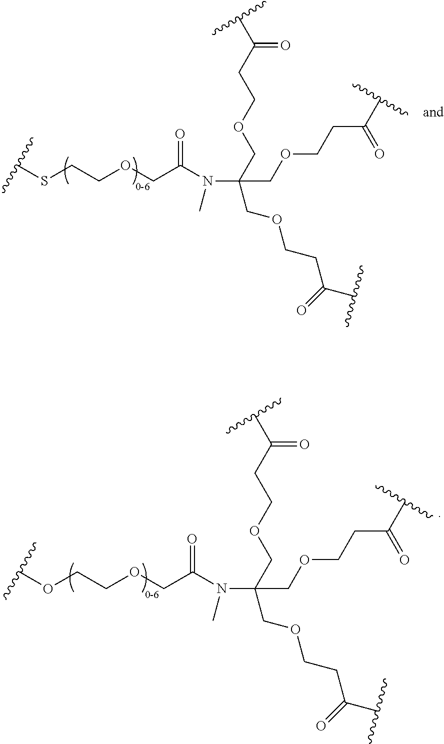

In certain embodiments Linker^{D} is selected from:

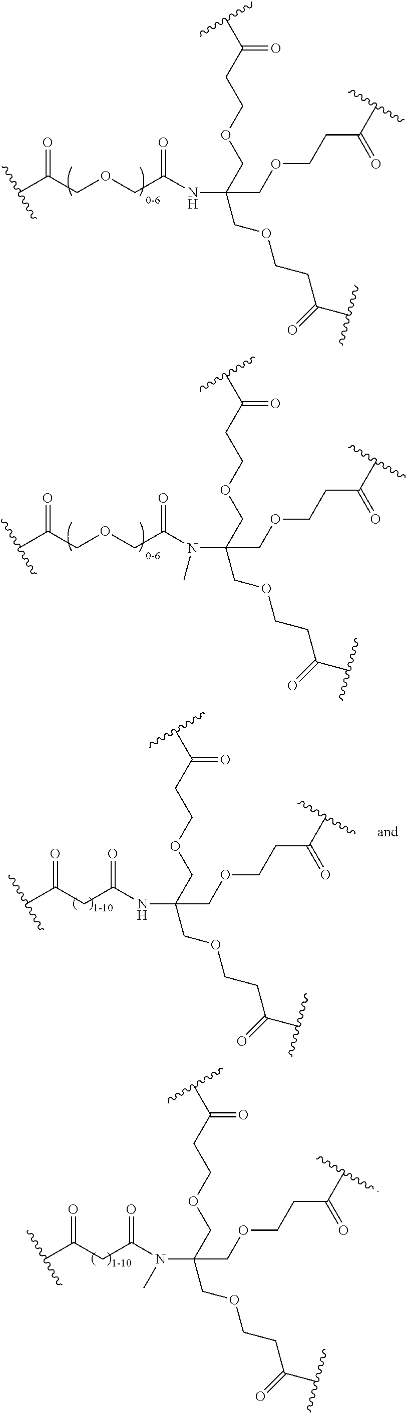

In certain embodiments Linker^{D} is selected from:

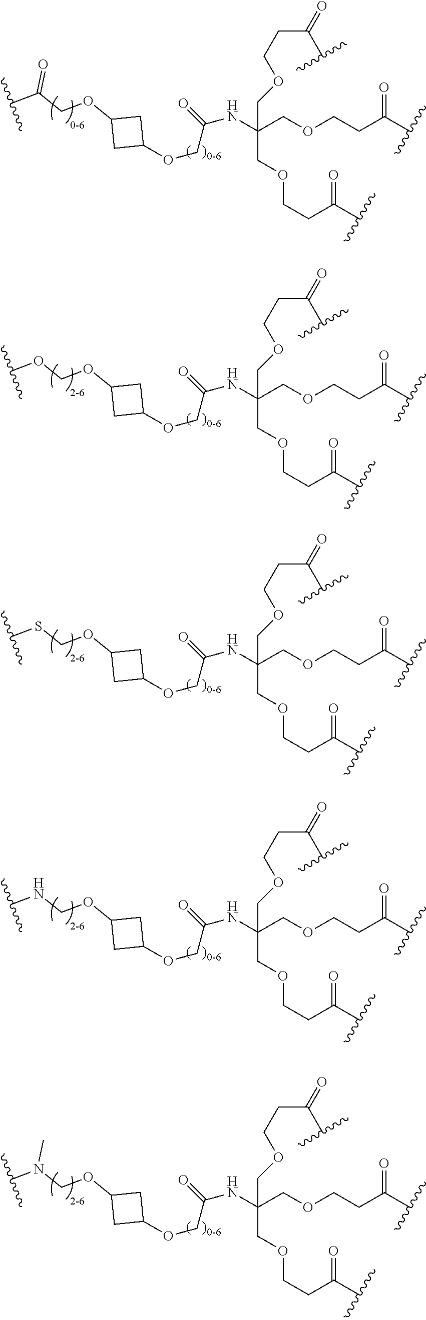

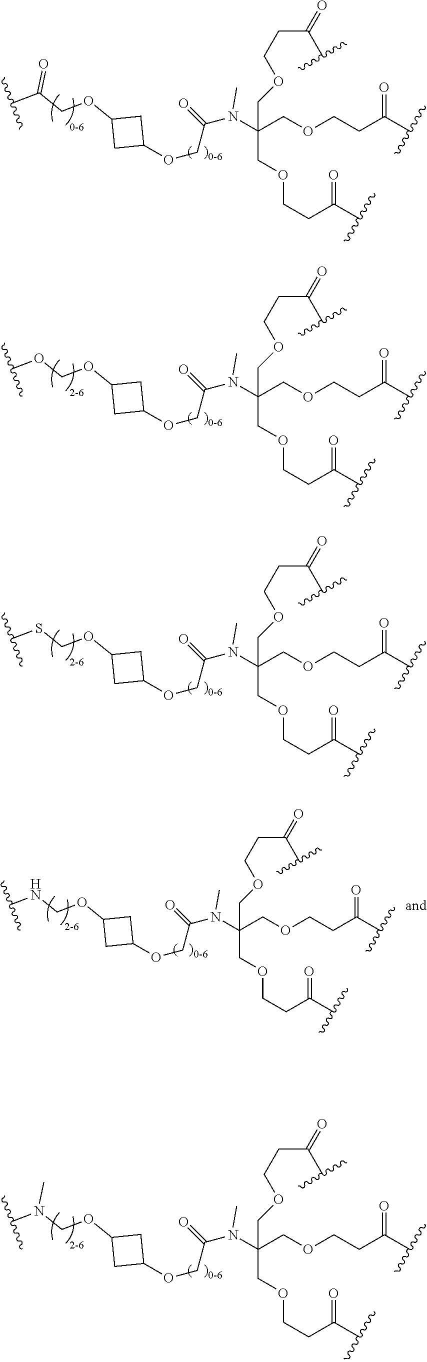

In certain embodiments Linker^{D} is selected from:

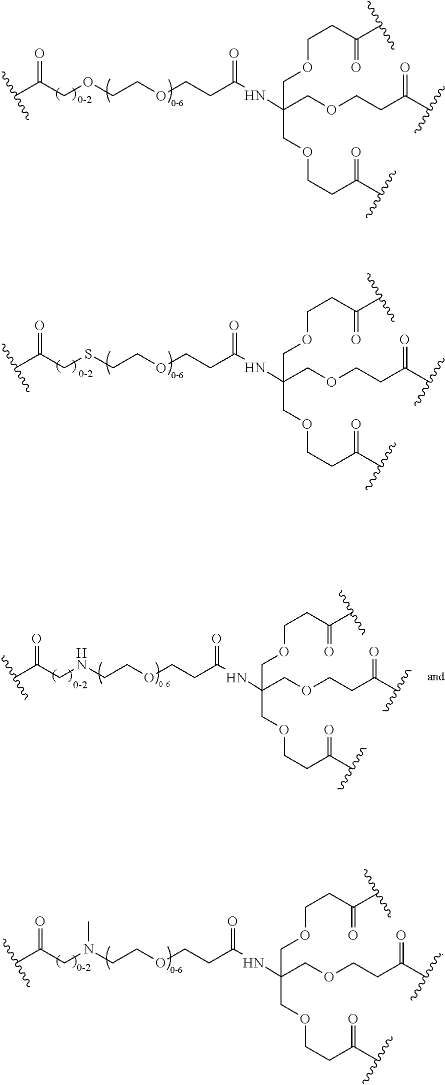

In certain embodiments Linker^{D} is selected from:

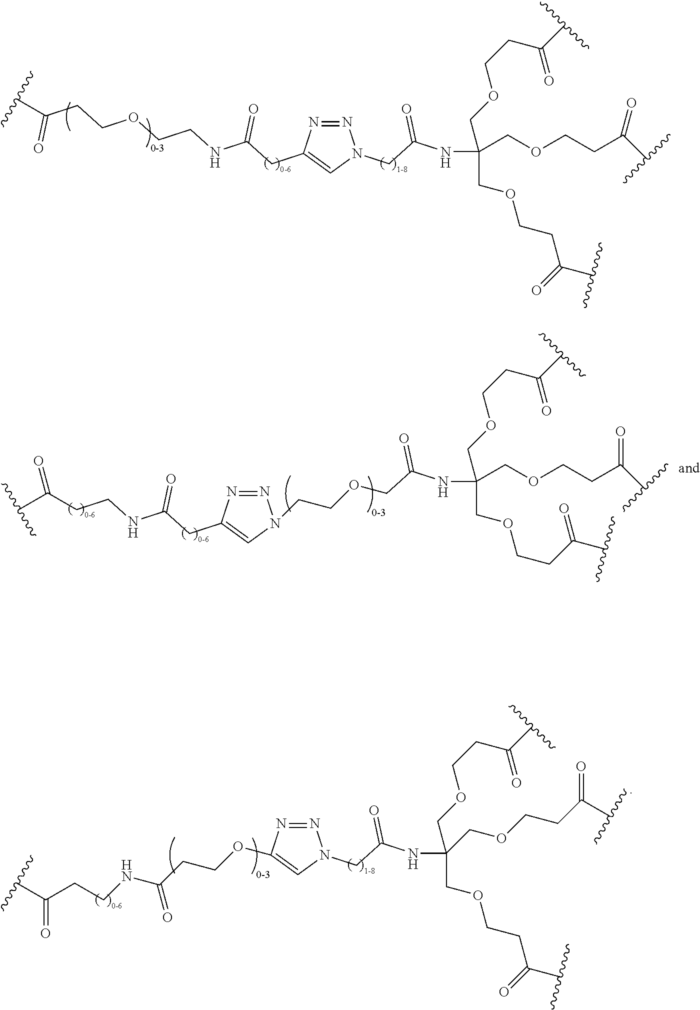

In certain embodiments Linker^{D} is selected from:

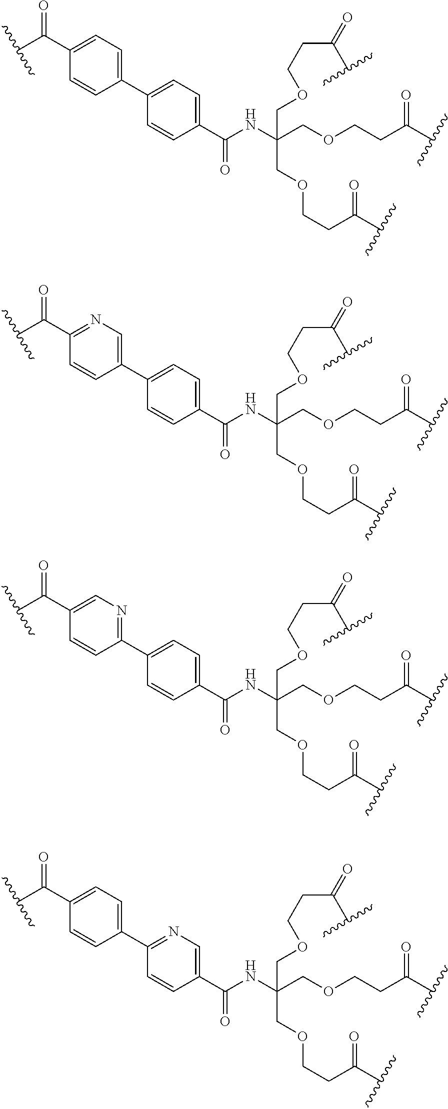 [SMILES: *C(=O)CCOCC(COCCC(*)=O)(COCCC(*)=O)NC(=O)c1ccc(-c2ccc(C(*)=O)cc2)cc1 ; *C(=O)CCOCC(COCCC(*)=O)(COCCC(*)=O)NC(=O)c1ccc(-c2ccc(C(*)=O)nc2)cc1 ; *C(=O)CCOCC(COCCC(*)=O)(COCCC(*)=O)NC(=O)c1ccc(-c2ccc(C(*)=O)cn2)cc1 ; *C(=O)CCOCC(COCCC(*)=O)(COCCC(*)=O)NC(=O)c1ccc(-c2ccc(C(*)=O)cc2)nc1]

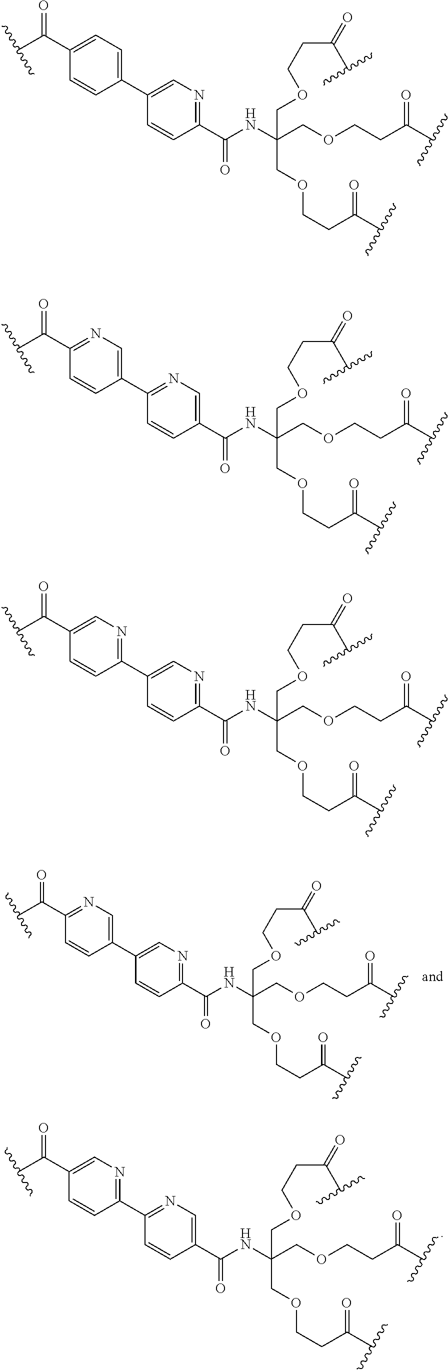 [SMILES: *C(=O)CCOCC(COCCC(*)=O)(COCCC(*)=O)NC(=O)c1ccc(-c2ccc(C(*)=O)cc2)cn1 ; *C(=O)CCOCC(COCCC(*)=O)(COCCC(*)=O)NC(=O)c1ccc(-c2ccc(C(*)=O)nc2)nc1 ; *C(=O)CCOCC(COCCC(*)=O)(COCCC(*)=O)NC(=O)c1ccc(-c2ccc(C(*)=O)cn2)cn1 ; *C(=O)CCOCC(COCCC(*)=O)(COCCC(*)=O)NC(=O)c1ccc(-c2ccc(C(*)=O)nc2)cn1 ; *C(=O)CCOCC(COCCC(*)=O)(COCCC(*)=O)NC(=O)c1ccc(-c2ccc(C(*)=O)cn2)nc1]

In certain embodiments, Linker^{A} or Linker^{B} is selected from:

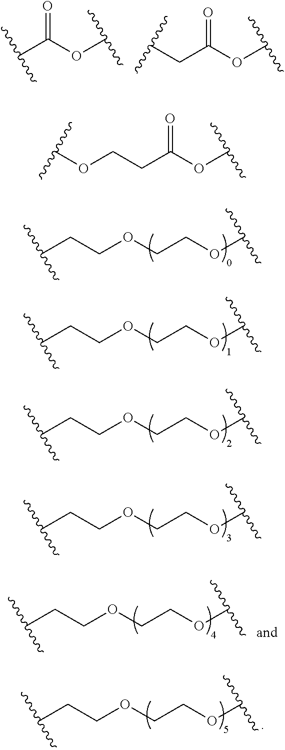

In certain embodiments, Linker^{A} or Linker^{B} is selected from:

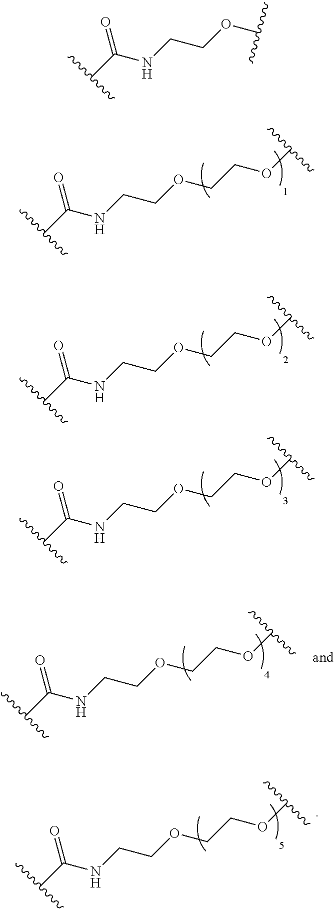

In certain embodiments, Linker^{A} or Linker^{B} is selected from:

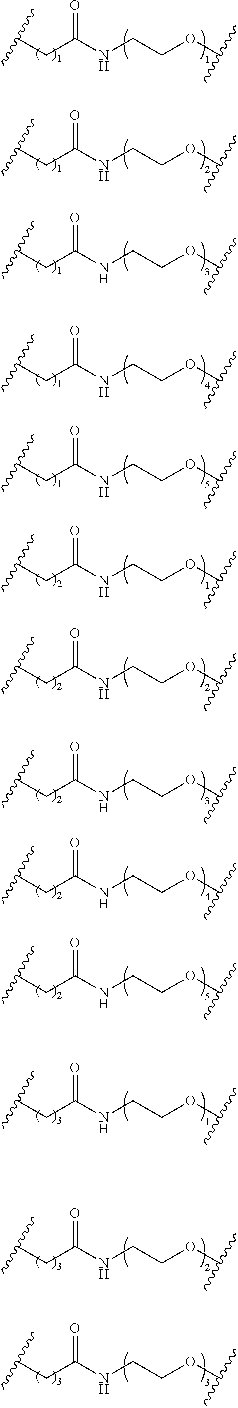

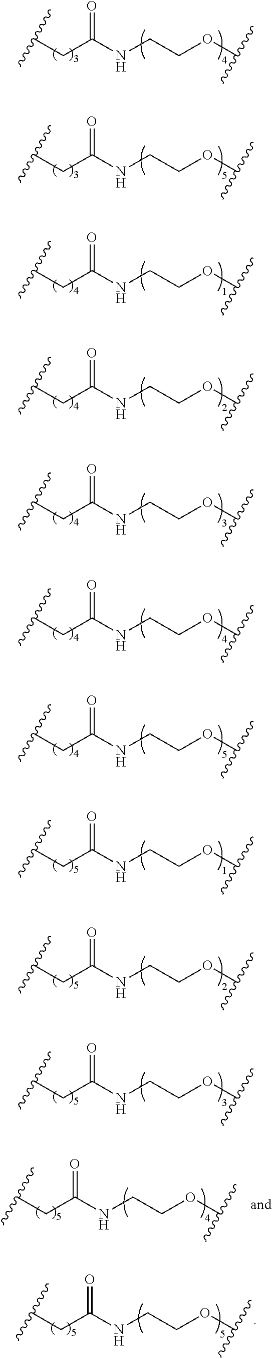

In certain embodiments, Linker^{A} or Linker is selected from:

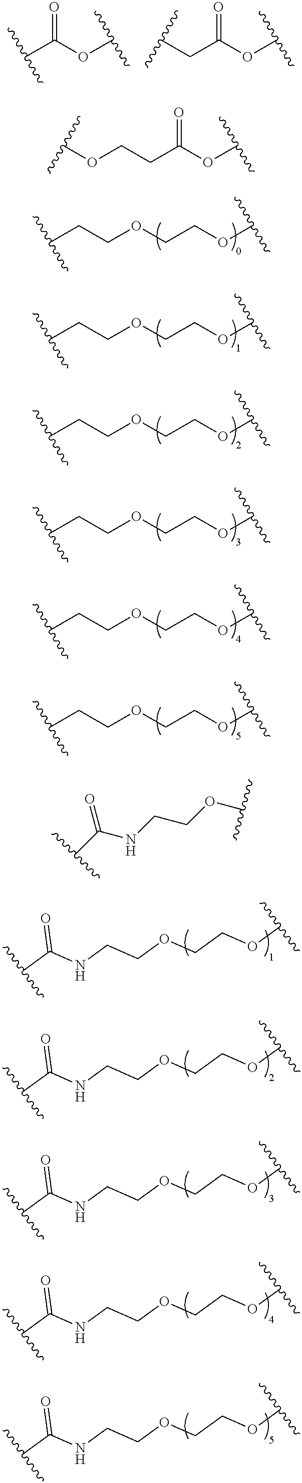

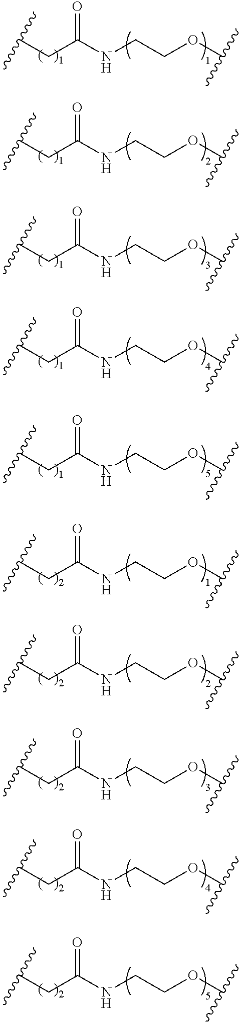

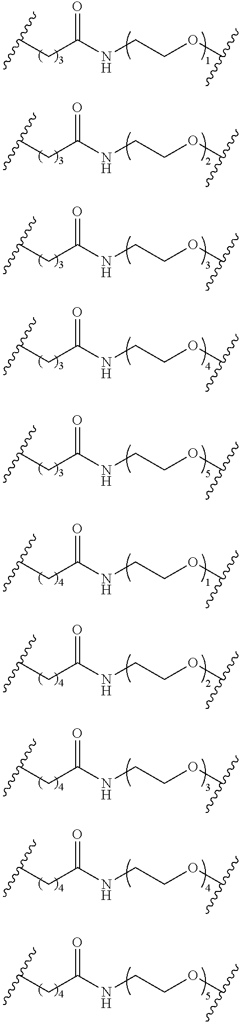

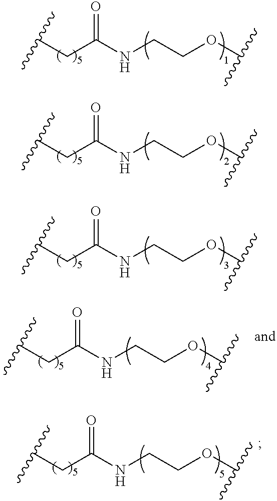

wherein each is optionally substituted with 1, 2, 3, or 4 substituents substituent selected from R²¹.

In certain embodiments, Linker^{A} or Linker^{B} is selected from:

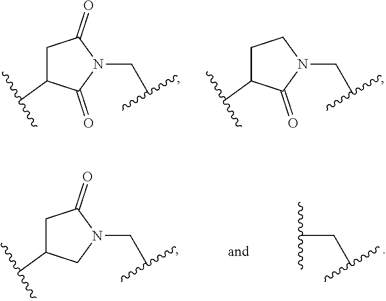

In certain embodiments, Linker^{A} or Linker^{B} is selected from:

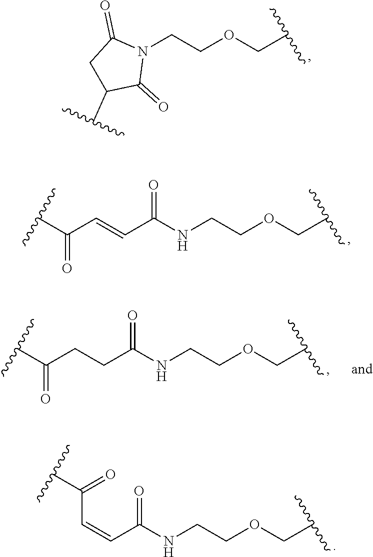 [SMILES: *COCCN1C(=O)CC(*)C1=O ; *COCCNC(=O)/C=C/C(*)=O ; *COCCNC(=O)CCC(*)=O ; *COCCNC(=O)/C=C\C(*)=O]

In certain embodiments, Linker^{A} or Linker^{B} is selected from:

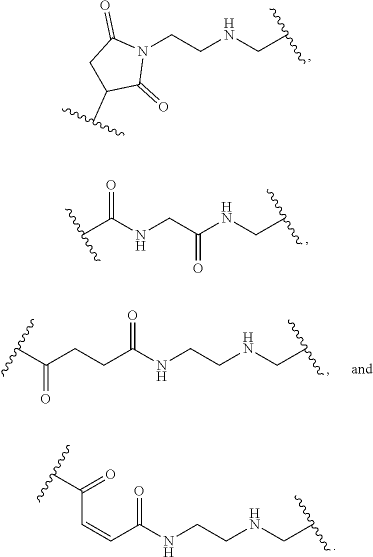 [SMILES: *CNCCN1C(=O)CC(*)C1=O ; *CNC(=O)CNC(*)=O ; *CNCCNC(=O)CCC(*)=O ; *CNCCNC(=O)/C=C\C(*)=O]

In certain embodiments, Linker^{A} or Linker^{B} is selected from:

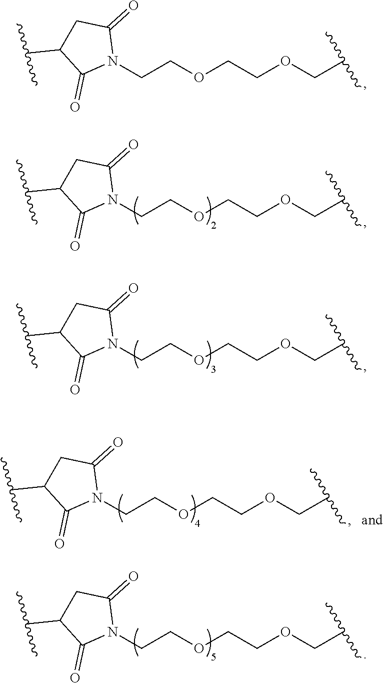

In certain embodiments, Linker^{A} or Linker^{B} is selected from:

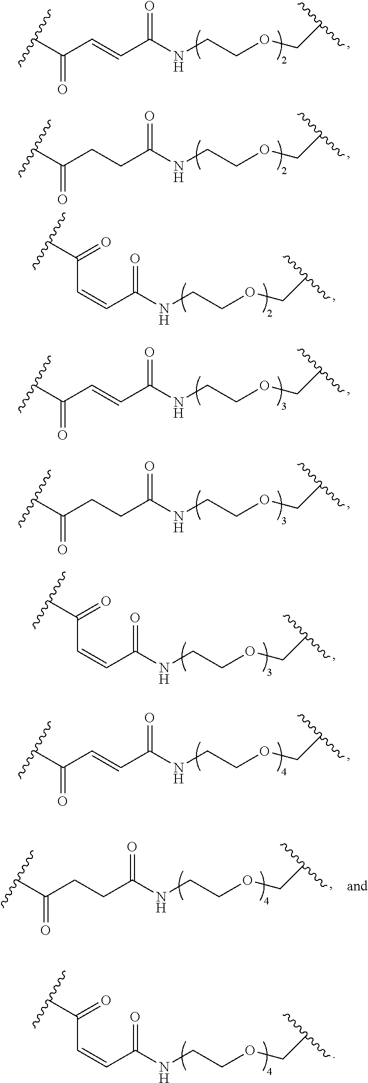

In certain embodiments, Linker^{A} or Linker^{B} is selected from:

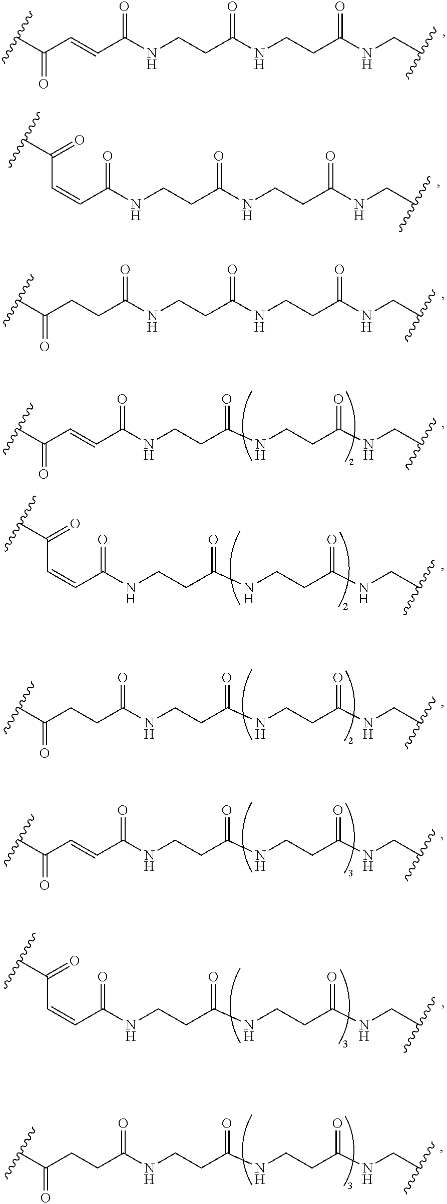

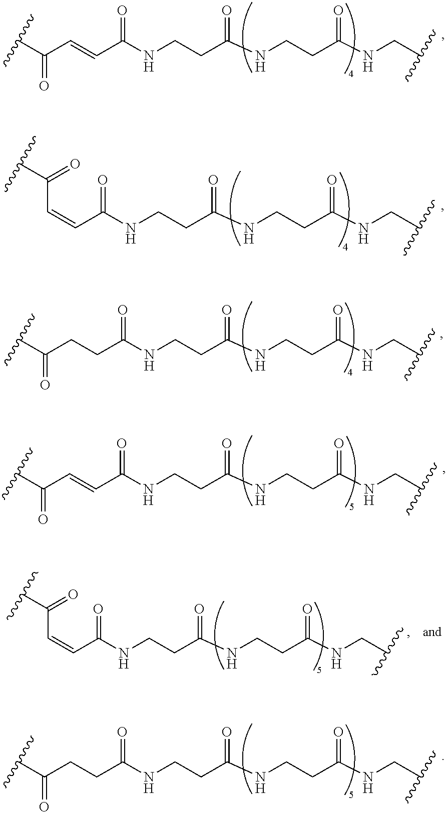

In certain embodiments, Linker^{A} or Linker^{B} is selected from:

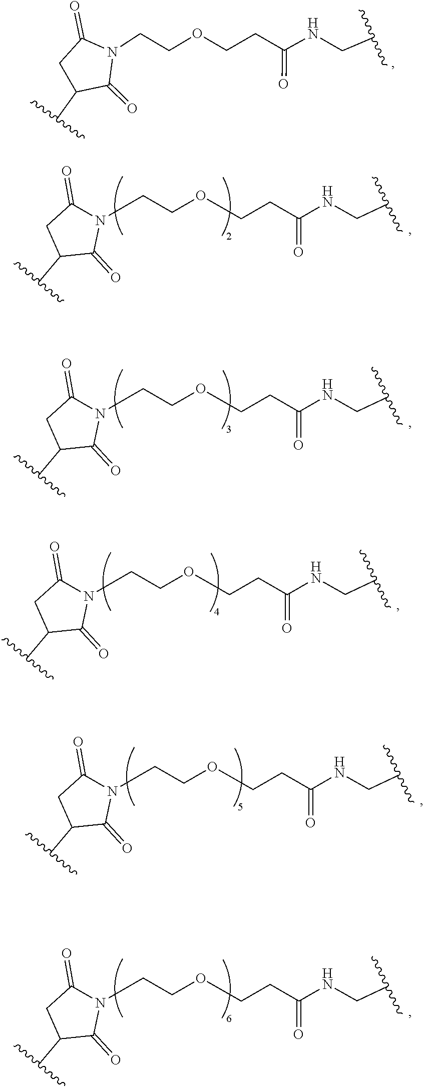

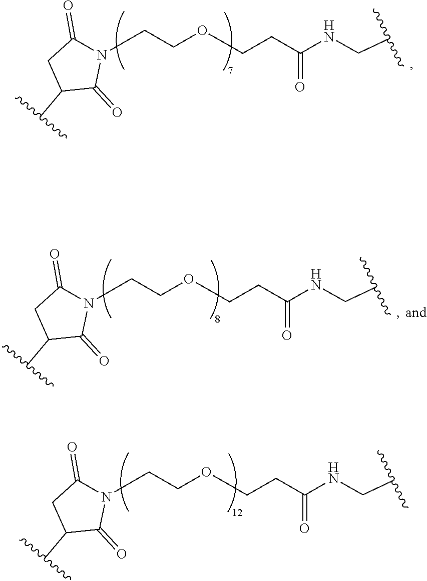

In certain embodiments, Linker^{A} or Linker^{B} is selected from:

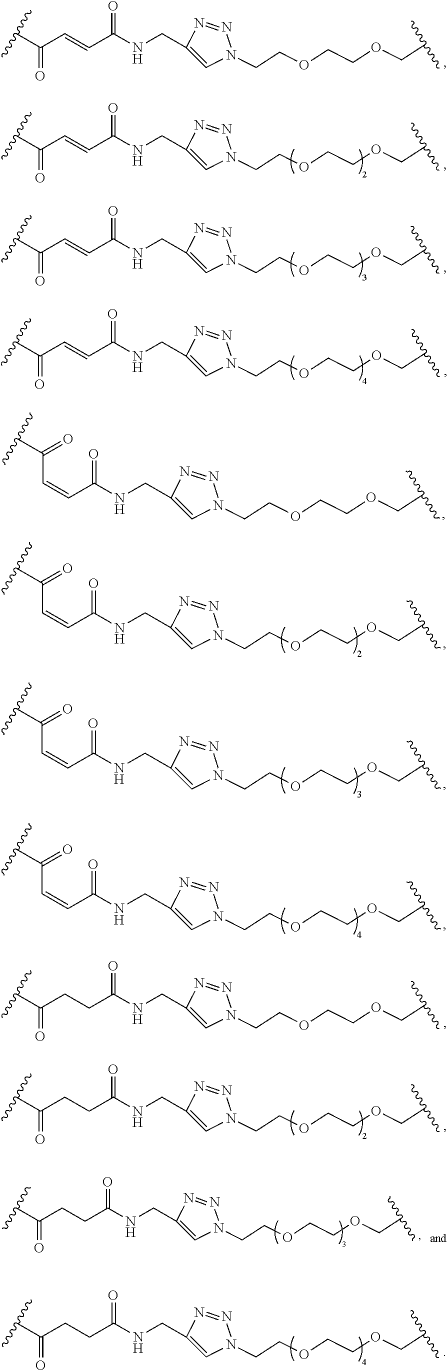

In certain embodiments, Linker^{A} or Linker^{B} is selected from:

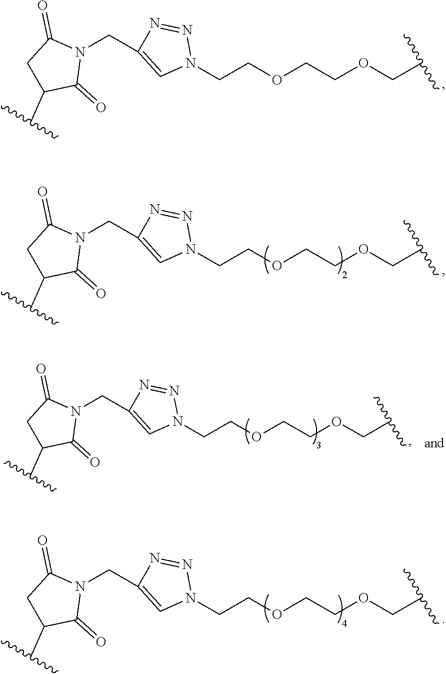

In certain embodiments, Linker^{A} or Linker^{B} is selected from:

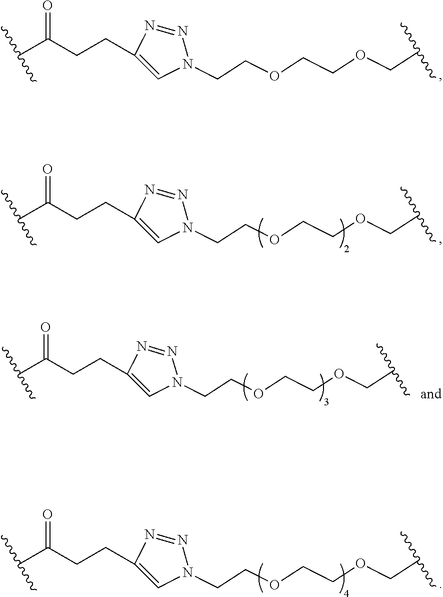

In certain embodiments, Linker^{A} or Linker^{B} is selected from:

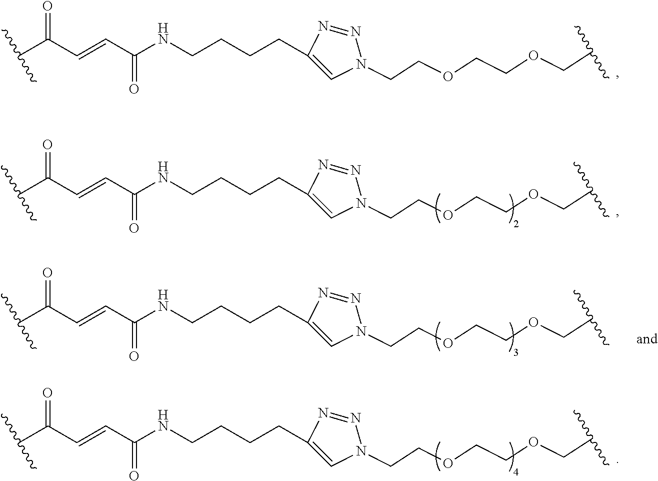

In certain embodiments, Linker^{A} or Linker^{B} is selected from:

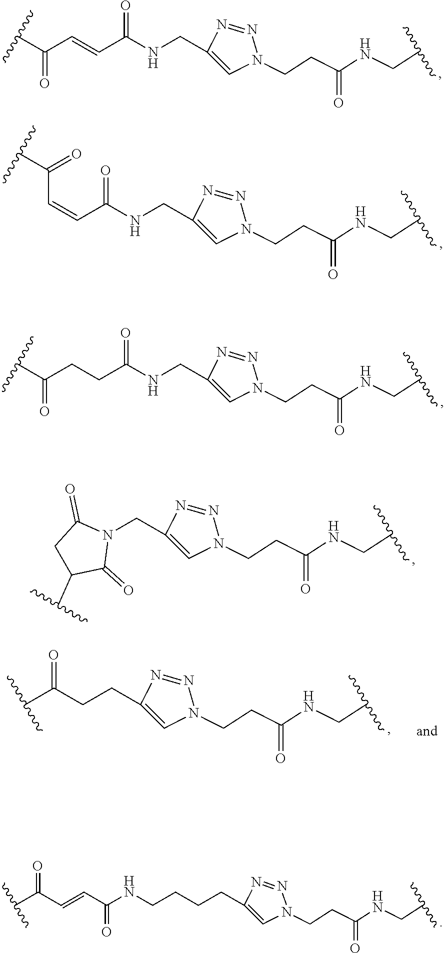 [SMILES: *CNC(=O)CCn1cc(CNC(=O)/C=C/C(*)=O)nn1 ; *CNC(=O)CCn1cc(CNC(=O)/C=C\C(*)=O)nn1 ; *CNC(=O)CCn1cc(CNC(=O)CCC(*)=O)nn1 ; *CNC(=O)CCn1cc(CN2C(=O)CC(*)C2=O)nn1 ; *CNC(=O)CCn1cc(CCC(*)=O)nn1 ; *CNC(=O)CCn1cc(CCCCNC(=O)/C=C/C(*)=O)nn1]

In certain embodiments, Linker^{A} or Linker^{B} is selected from:

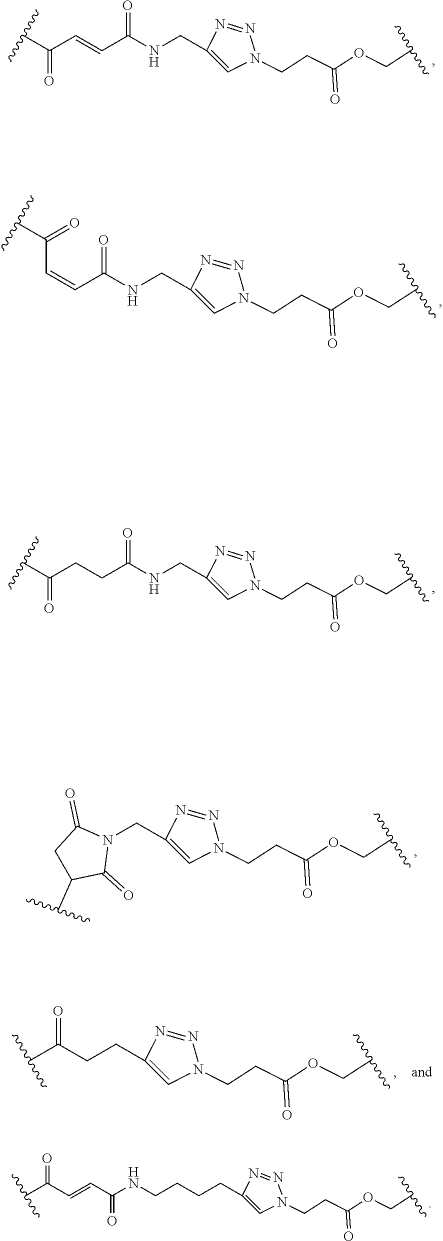 [SMILES: *COC(=O)CCn1cc(CNC(=O)/C=C/C(*)=O)nn1 ; *COC(=O)CCn1cc(CNC(=O)/C=C\C(*)=O)nn1 ; *COC(=O)CCn1cc(CNC(=O)CCC(*)=O)nn1 ; *COC(=O)CCn1cc(CN2C(=O)CC(*)C2=O)nn1 ; *COC(=O)CCn1cc(CCC(*)=O)nn1 ; *COC(=O)CCn1cc(CCCCNC(=O)/C=C/C(*)=O)nn1]

In certain embodiments, the Linker^{B} is selected from

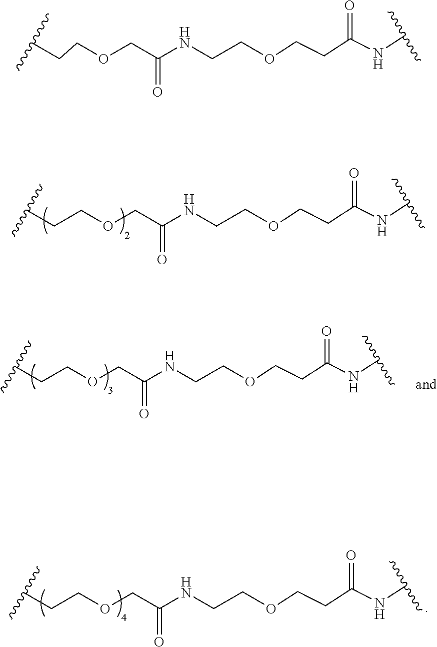

In certain embodiments, the Linker^{B} is selected from

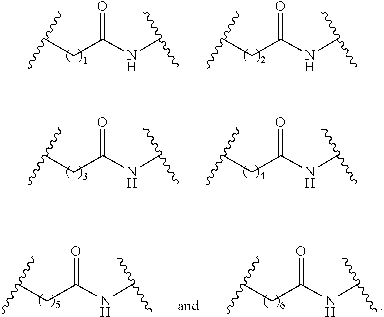

In certain embodiments, the Linker^{B} is selected from

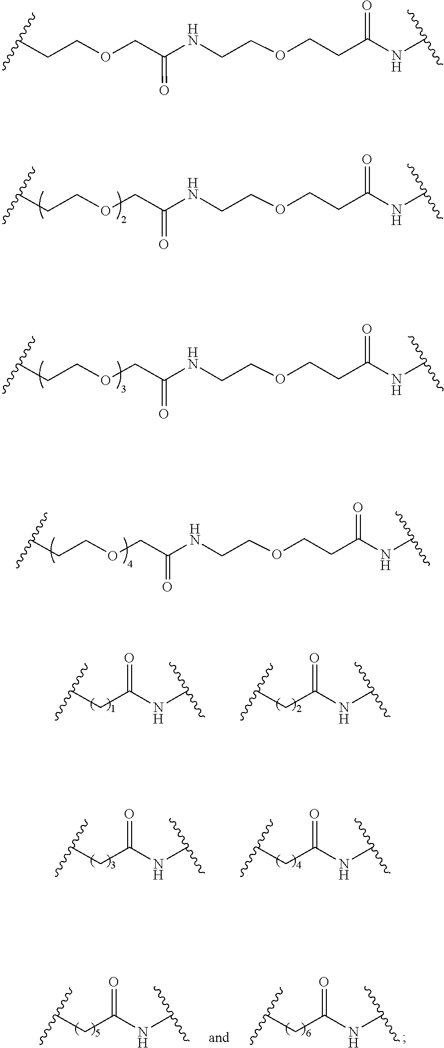

wherein each is optionally substituted with 1, 2, 3, or 4 substituents substituent selected from R²¹.

In certain embodiments Linker^{B} is selected from:

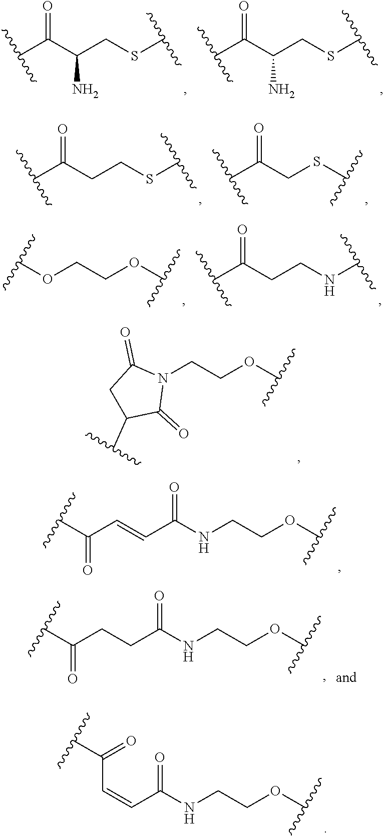

In certain embodiments Linker^{B} is selected from:

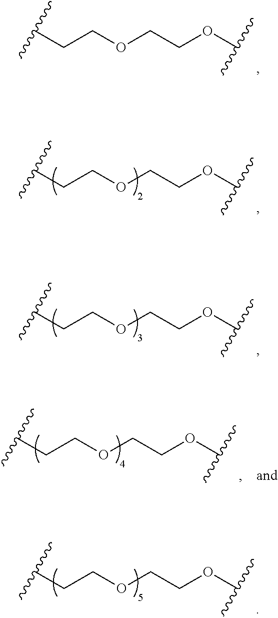

In certain embodiments Linker^{B} is selected from:

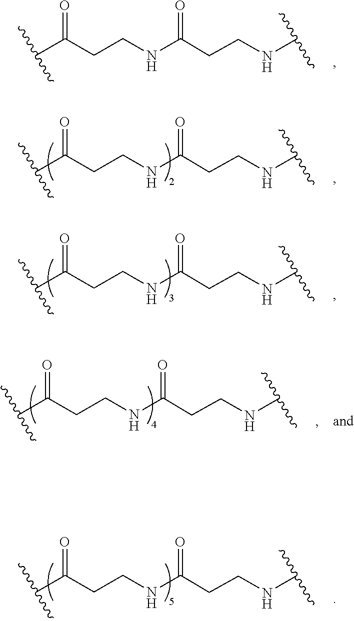

In certain embodiments Linker^{B} is selected from:

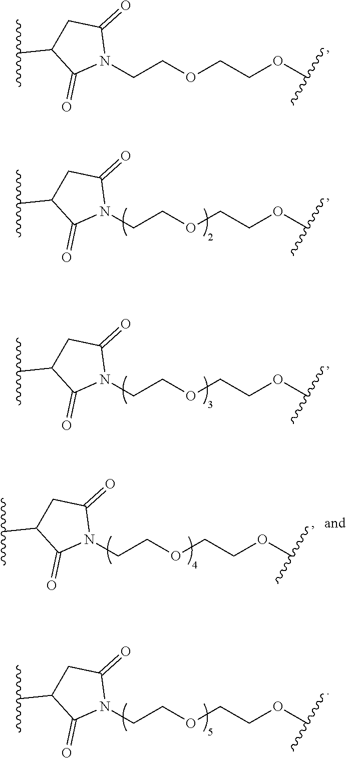

In certain embodiments Linker^{D} is selected from:

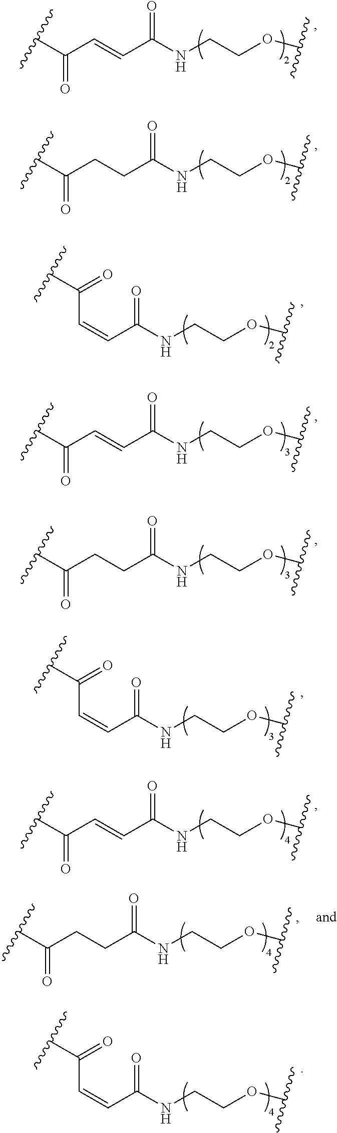

In certain embodiments Linker^{B} is selected from:

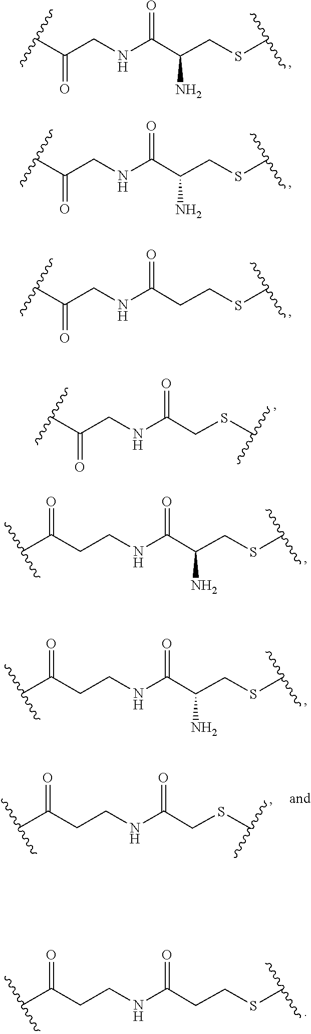

In certain embodiments Linker^{B} is selected from:

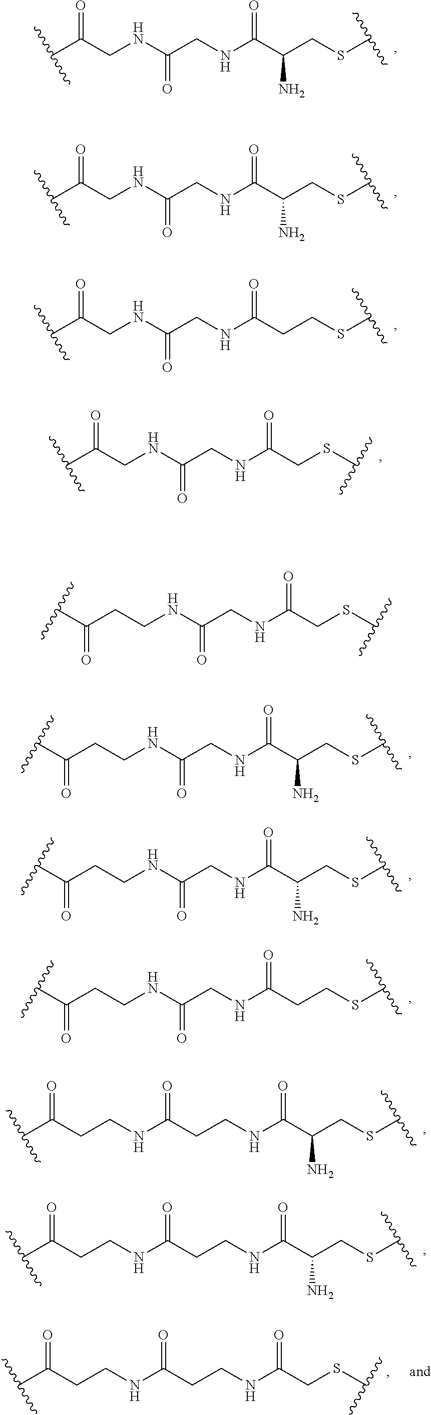

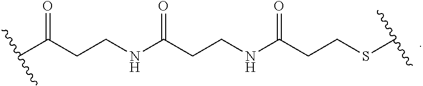 [SMILES: *SCCC(=O)NCCC(=O)NCCC(*)=O]

In certain embodiments Linker^{B} is selected from:

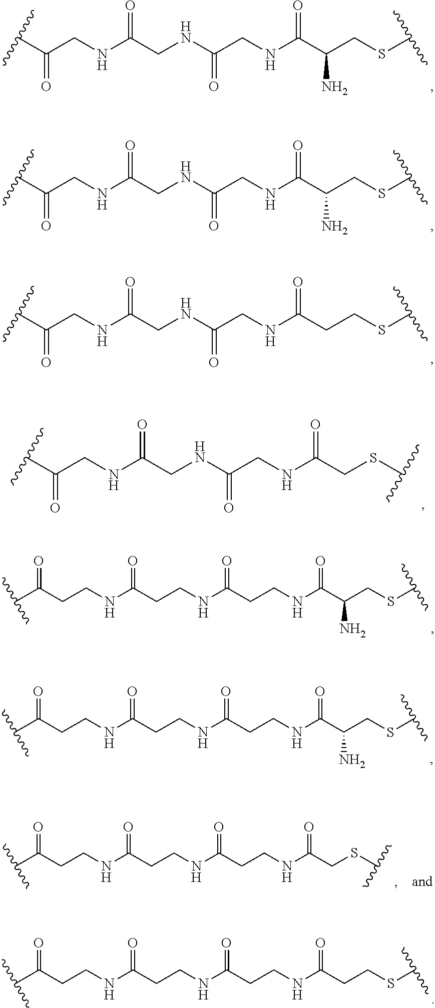

In certain embodiments Linker^{B}-Linker^{A} is selected from:

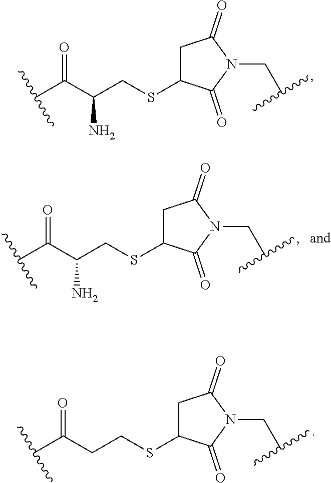 [SMILES: *CN1C(=O)CC(SC[C@@H](N)C(*)=O)C1=O ; *CN1C(=O)CC(SC[C@H](N)C(*)=O)C1=O ; *CN1C(=O)CC(SCCC(*)=O)C1=O]

In certain embodiments Linker^{B}-Linker^{A} is selected from:

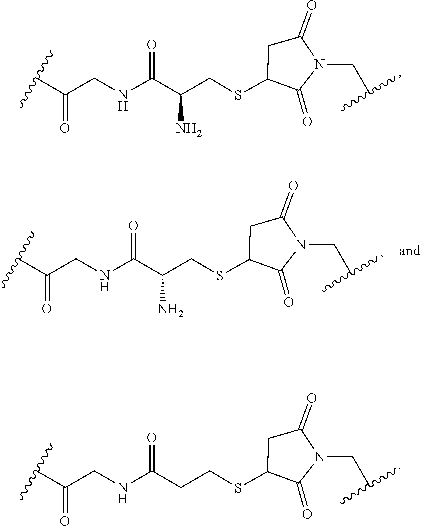 [SMILES: *CN1C(=O)CC(SC[C@@H](N)C(=O)NCC(*)=O)C1=O ; *CN1C(=O)CC(SC[C@H](N)C(=O)NCC(*)=O)C1=O ; *CN1C(=O)CC(SCCC(=O)NCC(*)=O)C1=O]

In certain embodiments, the Linker^{C} is selected from

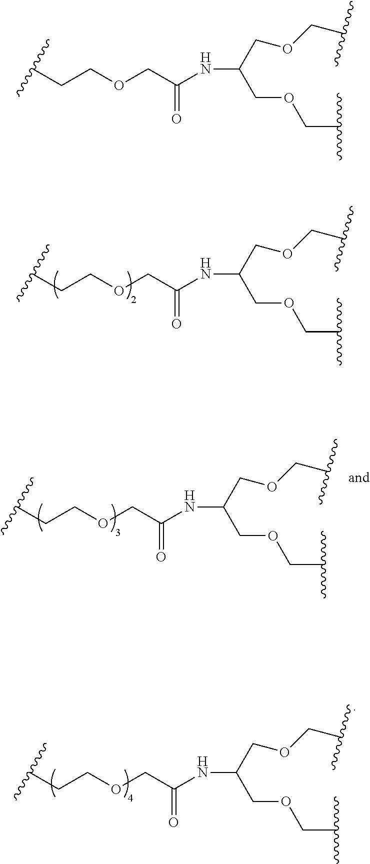

In certain embodiments, the Linker^{C} is selected from

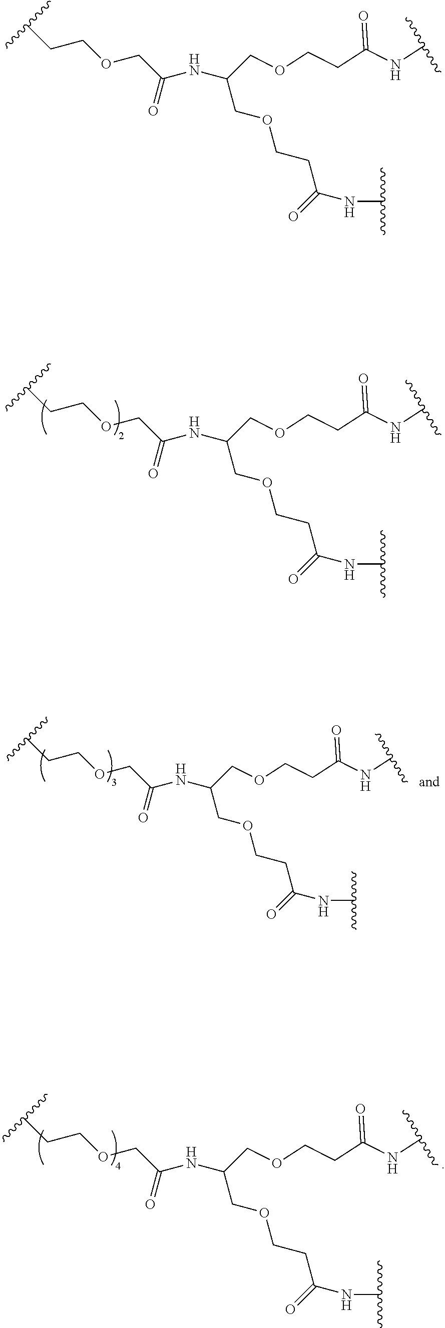

In certain embodiments, the Linker^{C} is selected from

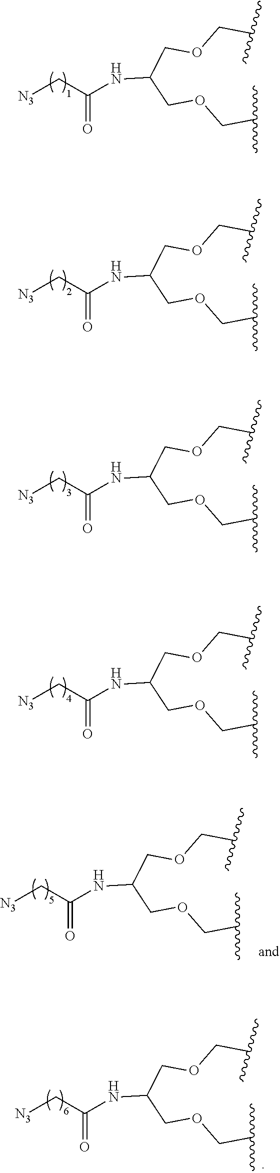

In certain embodiments, the Linker^{C} is selected from

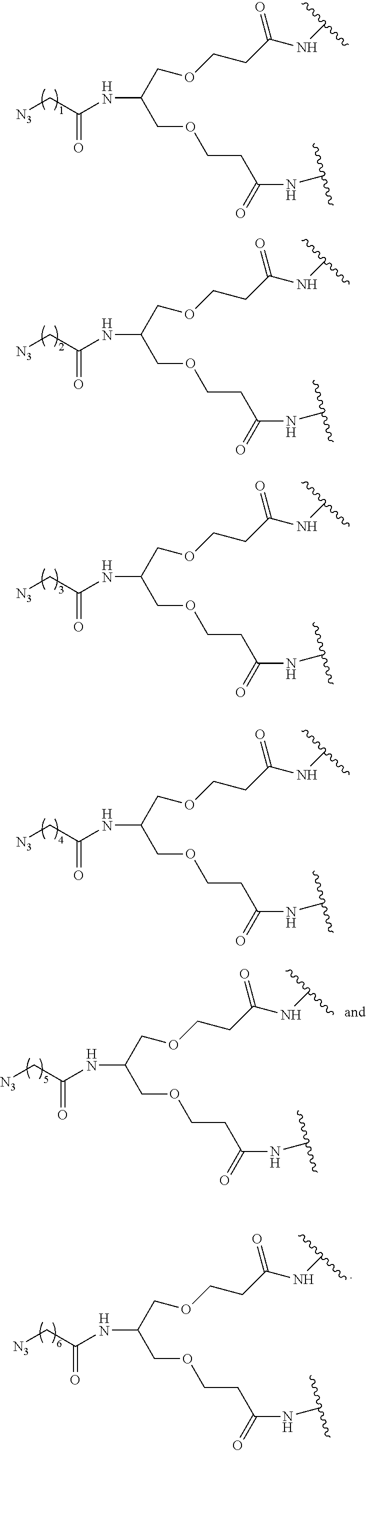

In certain embodiments, the Linker^{C} is selected from

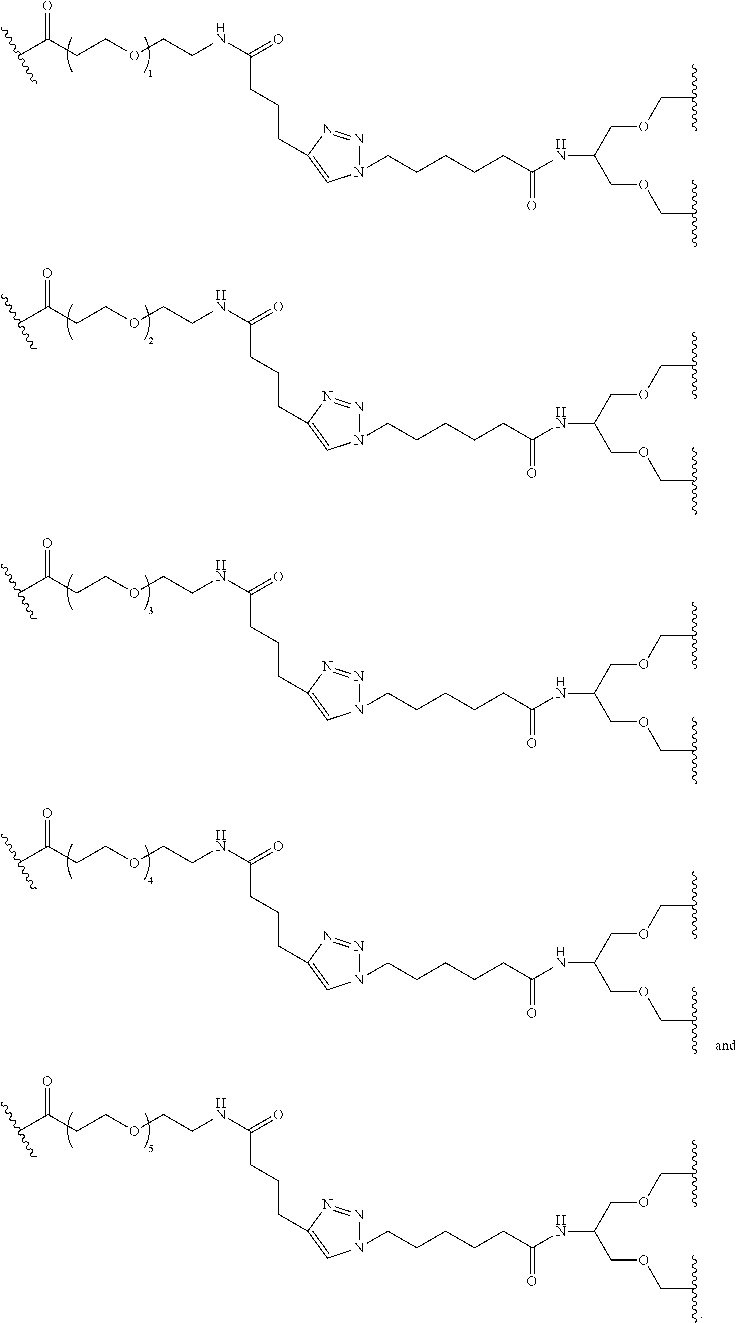

In certain embodiments, the Linker^{C} is selected from

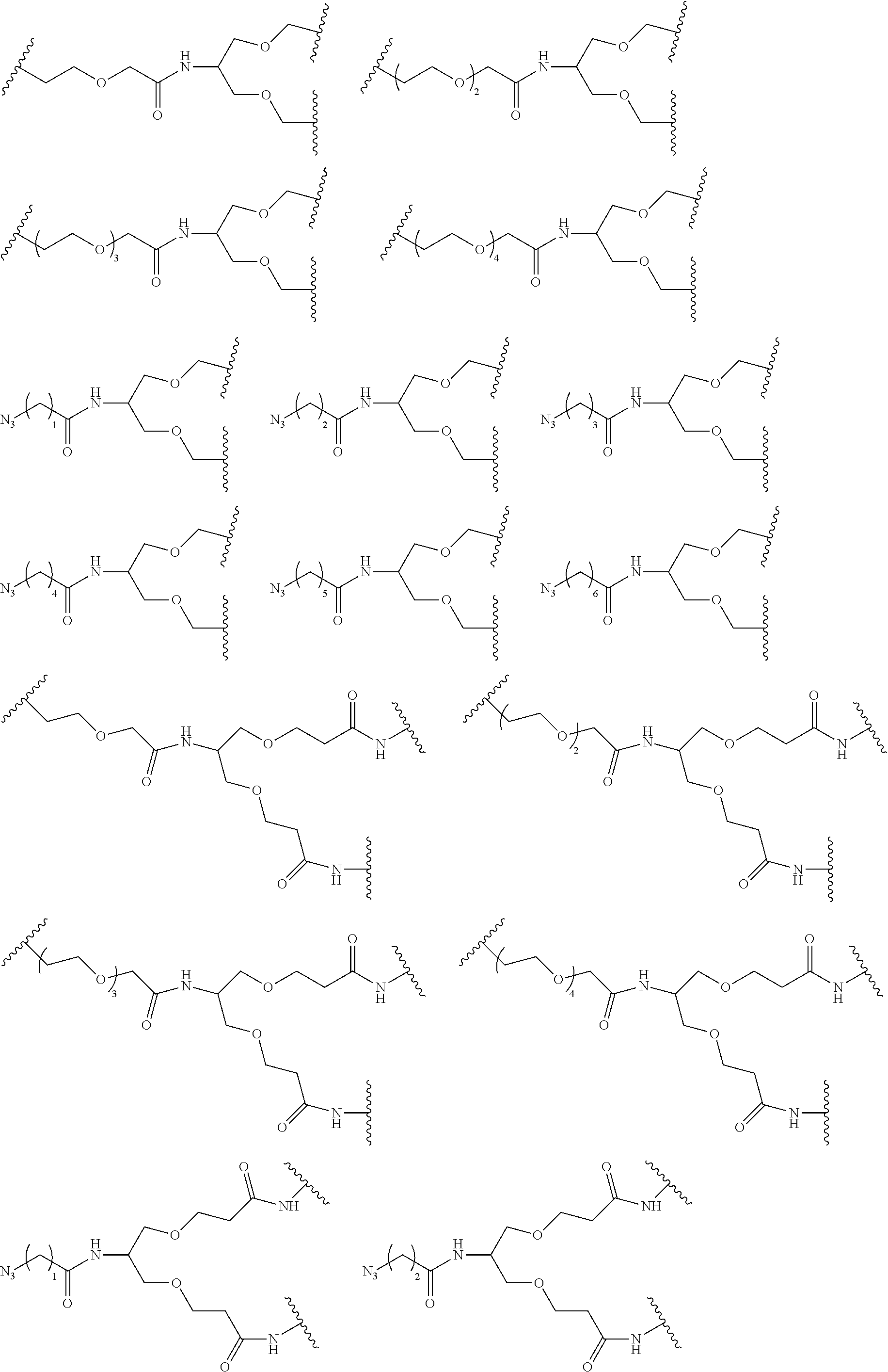

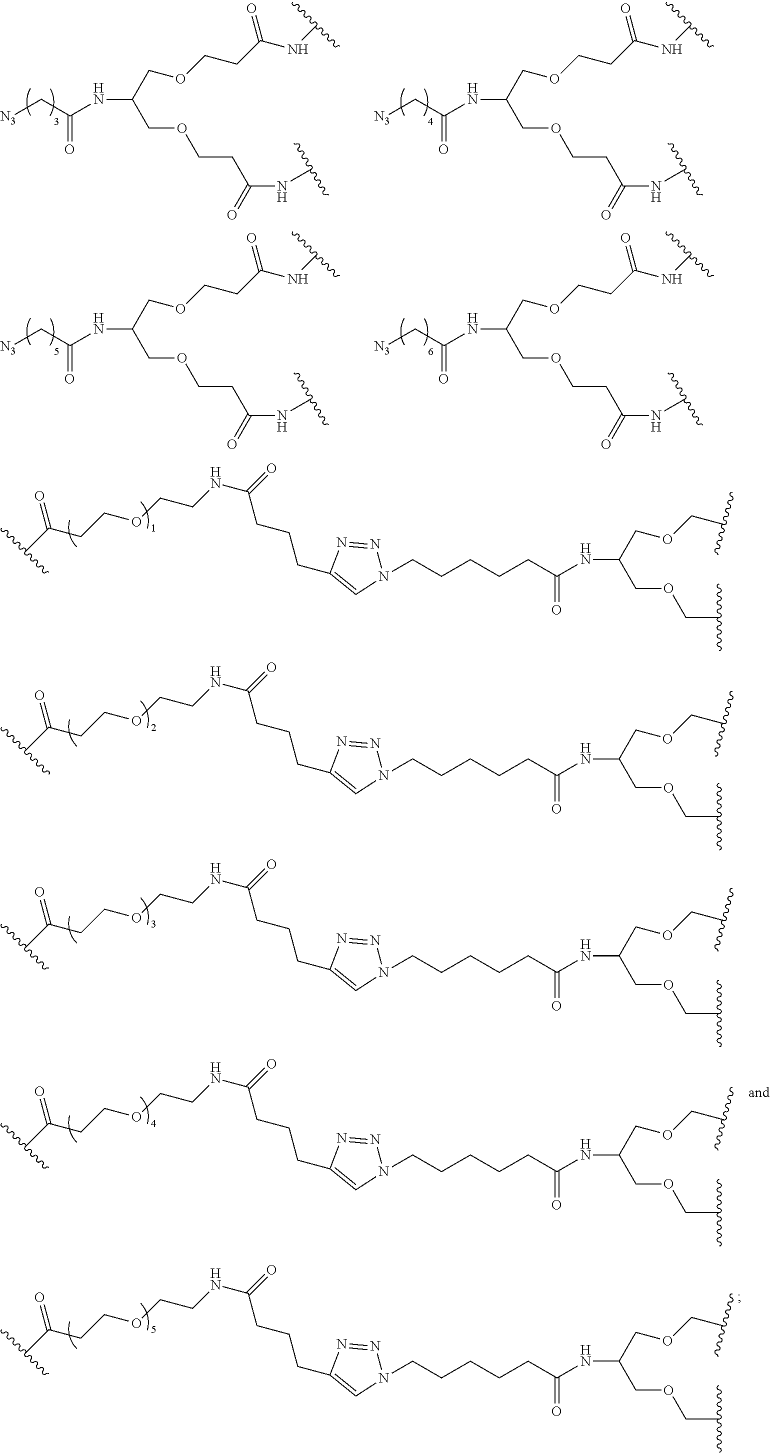

wherein each is optionally substituted with 1, 2, 3, or 4 substituents substituent selected from R²¹.

In certain embodiments Linker^{C} is selected from:

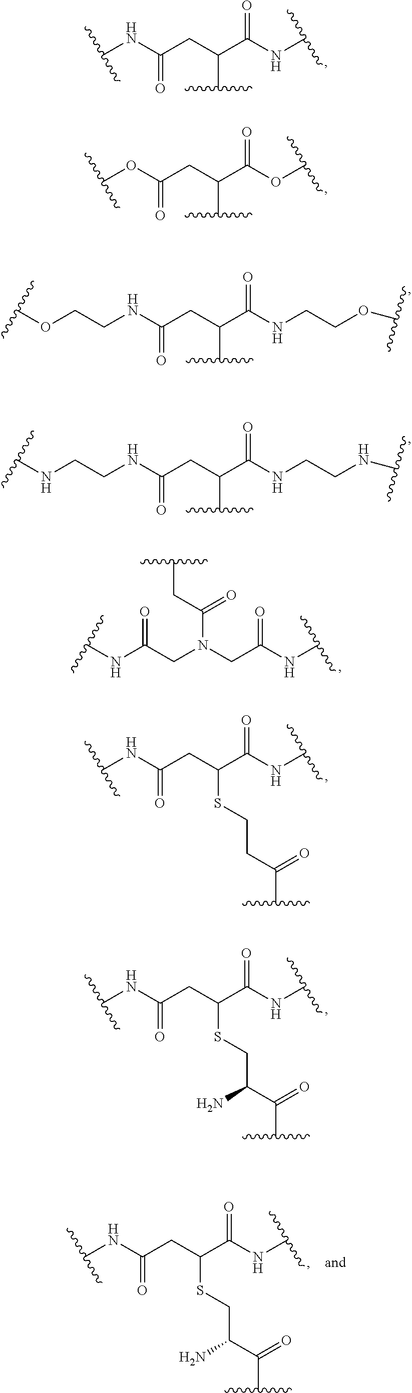

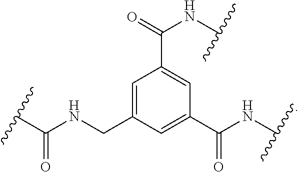 [SMILES: *NC(=O)c1cc(CNC(*)=O)cc(C(=O)N*)c1]

In certain embodiments Linker^{C} is selected from:

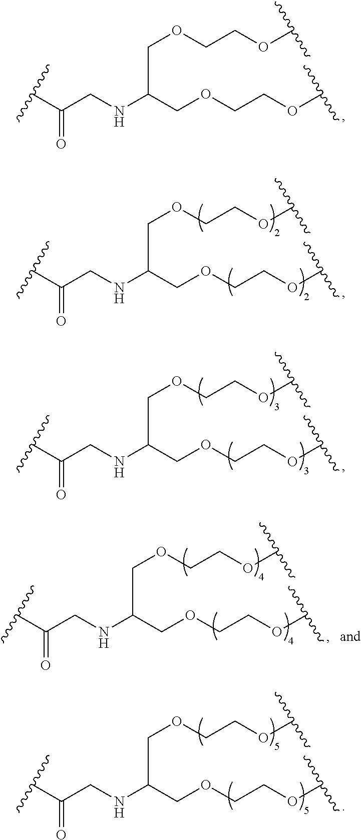

In certain embodiments Linker^{C} is selected from:

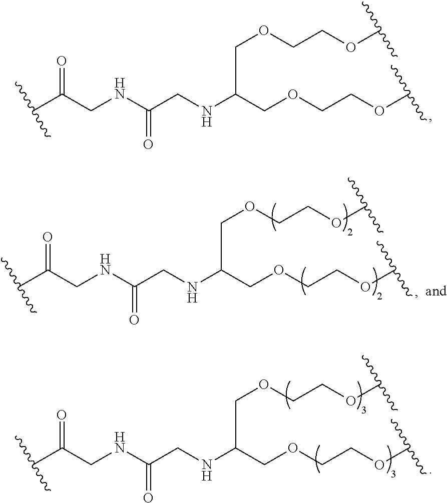

In certain embodiments Linker^{C} is selected from:

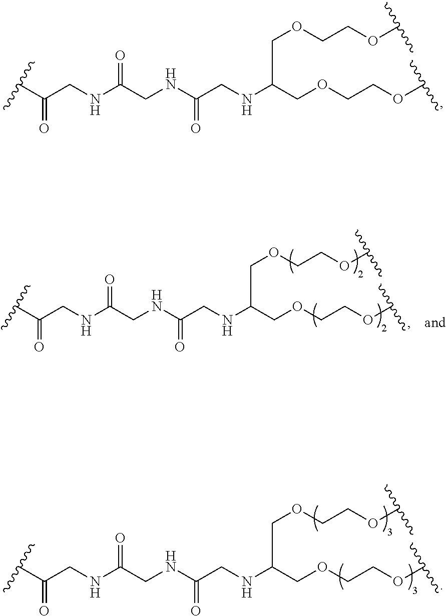

In certain embodiments Linker^{C} is selected from:

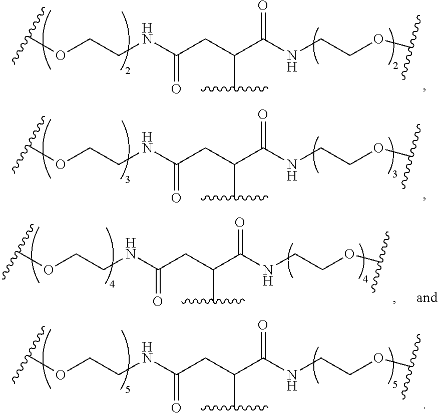

In certain embodiments Linker^{C} is selected from:

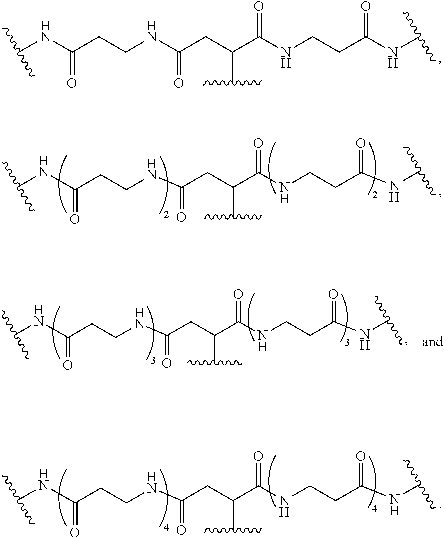

In certain embodiments Linker^{C} is selected from:

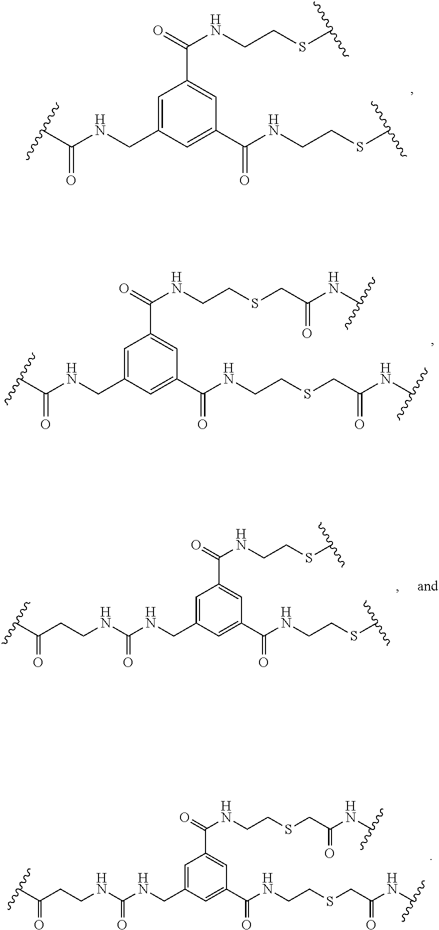 [SMILES: *SCCNC(=O)c1cc(CNC(*)=O)cc(C(=O)NCCS*)c1 ; *NC(=O)CSCCNC(=O)c1cc(CNC(*)=O)cc(C(=O)NCCSCC(=O)N*)c1 ; *SCCNC(=O)c1cc(CNC(=O)NCCC(*)=O)cc(C(=O)NCCS*)c1 ; *NC(=O)CSCCNC(=O)c1cc(CNC(=O)NCCC(*)=O)cc(C(=O)NCCSCC(=O)N*)c1]

In certain embodiments Linker^{C}-(Linker^{A})₂ is selected from:

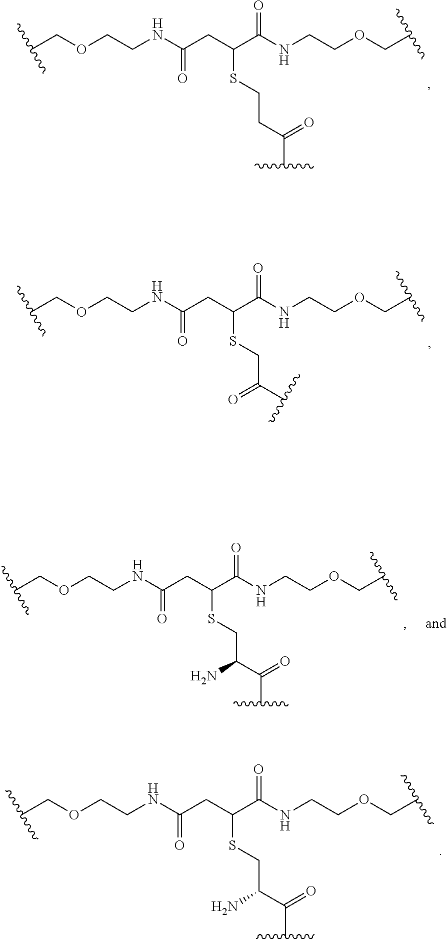 [SMILES: *COCCNC(=O)CC(SCCC(*)=O)C(=O)NCCOC* ; *COCCNC(=O)CC(SCC(*)=O)C(=O)NCCOC* ; *COCCNC(=O)CC(SC[C@H](N)C(*)=O)C(=O)NCCOC* ; *COCCNC(=O)CC(SC[C@@H](N)C(*)=O)C(=O)NCCOC*]

In certain embodiments Linker^{C}-(Linker^{A})₂ is selected from:

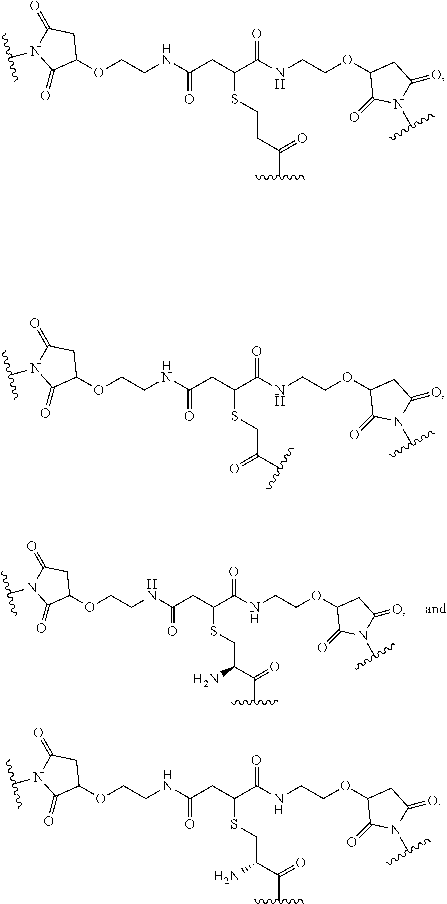 [SMILES: *C(=O)CCSC(CC(=O)NCCOC1CC(=O)N(*)C1=O)C(=O)NCCOC1CC(=O)N(*)C1=O ; *C(=O)CSC(CC(=O)NCCOC1CC(=O)N(*)C1=O)C(=O)NCCOC1CC(=O)N(*)C1=O ; *C(=O)[C@@H](N)CSC(CC(=O)NCCOC1CC(=O)N(*)C1=O)C(=O)NCCOC1CC(=O)N(*)C1=O ; *C(=O)[C@H](N)CSC(CC(=O)NCCOC1CC(=O)N(*)C1=O)C(=O)NCCOC1CC(=O)N(*)C1=O]

In certain embodiments Linker^{C}-(Linker^{A})₂ is selected from:

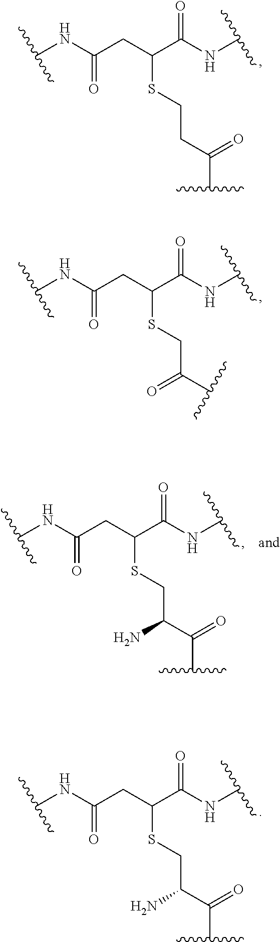 [SMILES: *NC(=O)CC(SCCC(*)=O)C(=O)N* ; *NC(=O)CC(SCC(*)=O)C(=O)N* ; *NC(=O)CC(SC[C@H](N)C(*)=O)C(=O)N* ; *NC(=O)CC(SC[C@@H](N)C(*)=O)C(=O)N*]

In certain embodiments Linker^{C}-(Linker^{A})₂ is selected from:

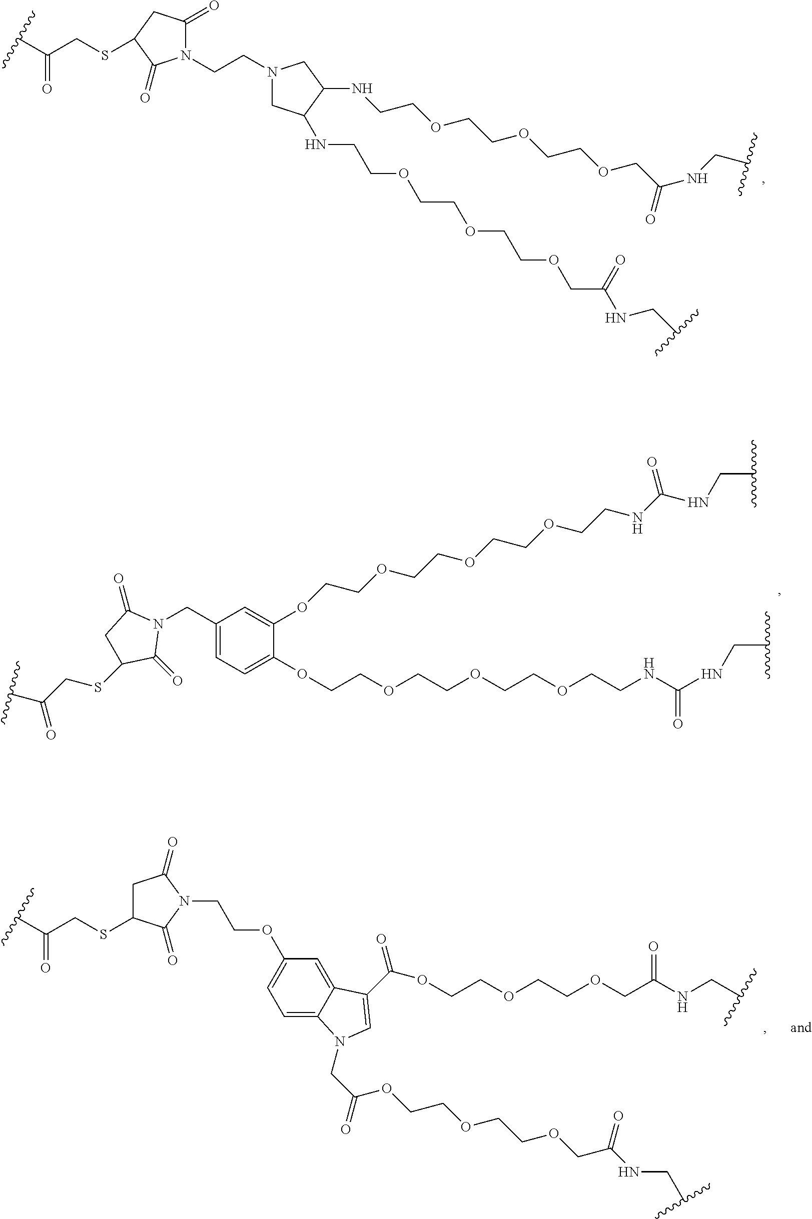 [SMILES: *CNC(=O)COCCOCCOCCNC1CN(CCN2C(=O)CC(SCC(*)=O)C2=O)CC1NCCOCCOCCOCC(=O)NC* ; *CNC(=O)NCCOCCOCCOCCOc1ccc(CN2C(=O)CC(SCC(*)=O)C2=O)cc1OCCOCCOCCOCCNC(=O)NC* ; *CNC(=O)COCCOCCOC(=O)Cn1cc(C(=O)OCCOCCOCC(=O)NC*)c2cc(OCCN3C(=O)CC(SCC(*)=O)C3=O)ccc21]

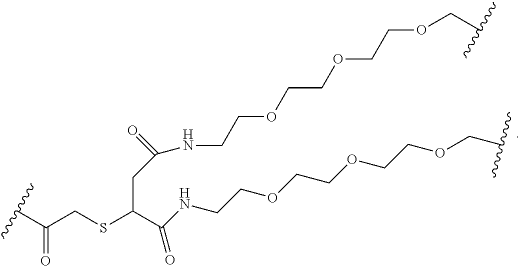 [SMILES: *COCCOCCOCCNC(=O)CC(SCC(*)=O)C(=O)NCCOCCOCCOC*]

In certain embodiments, the Linker^{D} is selected from

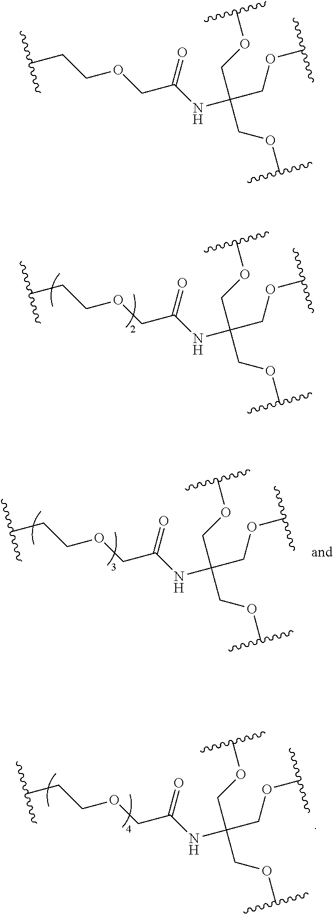

In certain embodiments, the Linker^{D} is selected from

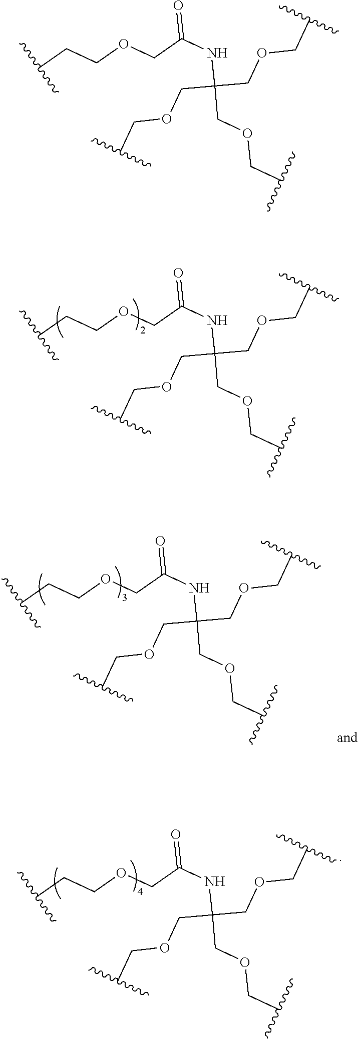

In certain embodiments, the Linker^{D} is selected from

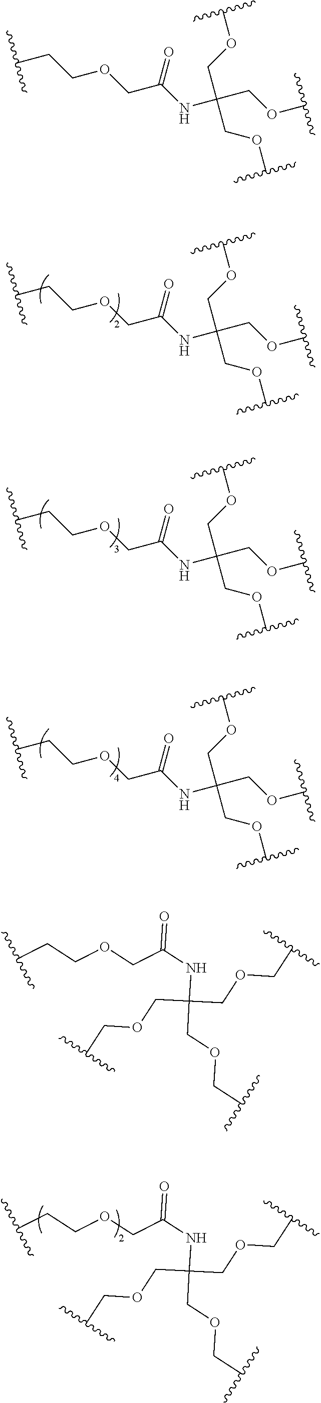

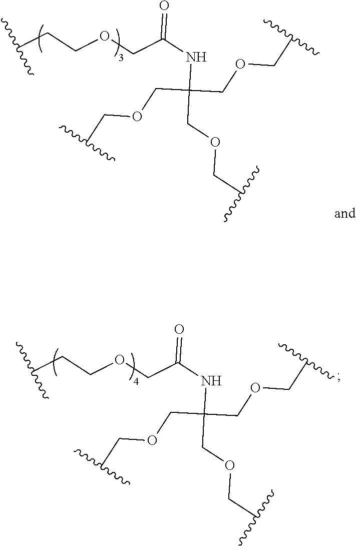

wherein each is optionally substituted with 1, 2, 3, or 4 substituents are selected from R²¹.

In certain embodiments, Linker^{B}-(Linker^{A}) is selected from

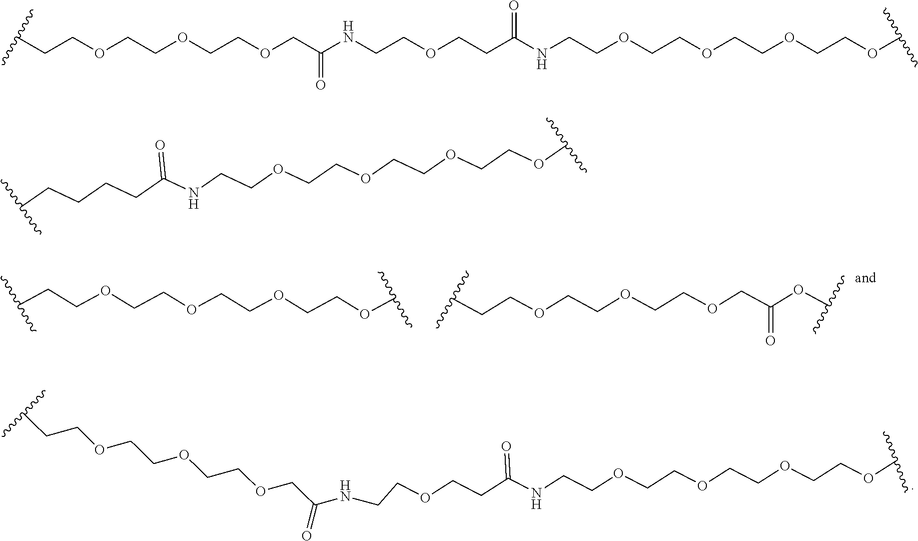 [SMILES: *CCOCCOCCOCC(=O)NCCOCCC(=O)NCCOCCOCCOCCO* ; *CCCCC(=O)NCCOCCOCCOCCO* ; *CCOCCOCCOCCO* ; *CCOCCOCCOCC(=O)O* ; *CCOCCOCCOCC(=O)NCCOCCC(=O)NCCOCCOCCOCCO*]

In certain embodiments, Linker^{C}-(Linker^{A}) is selected from

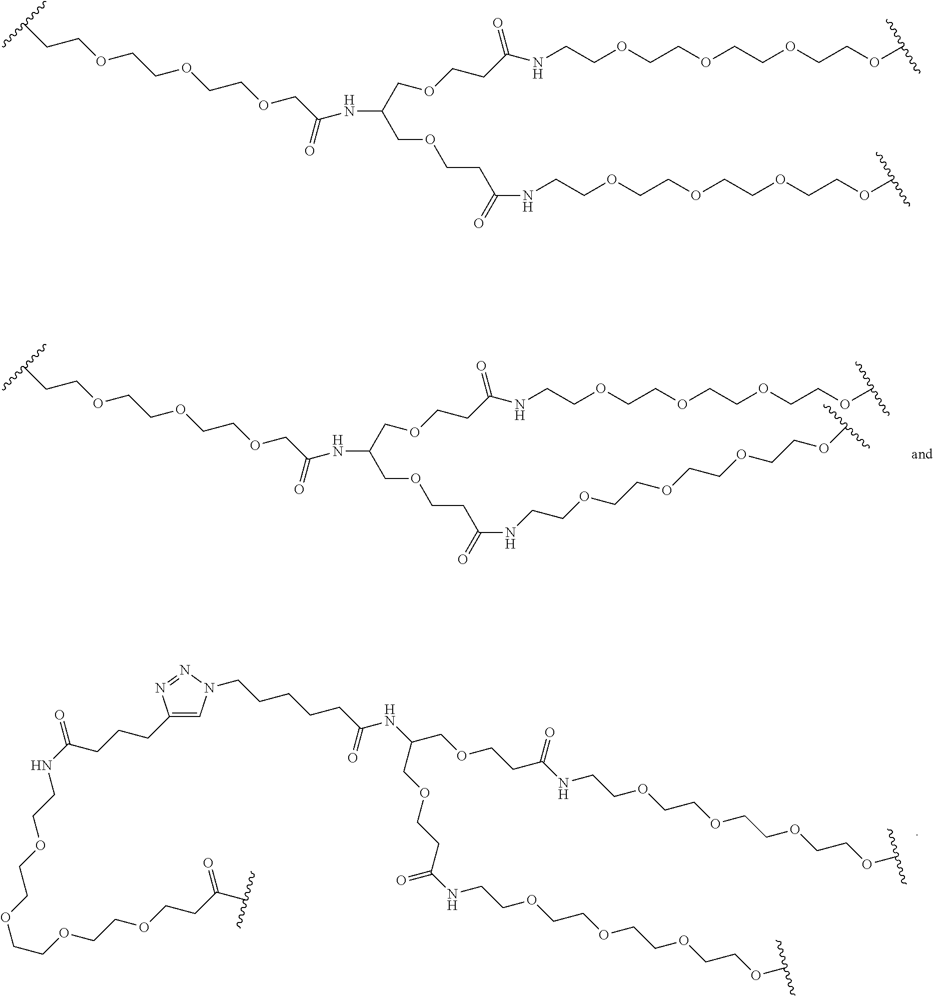 [SMILES: *CCOCCOCCOCC(=O)NC(COCCC(=O)NCCOCCOCCOCCO*)COCCC(=O)NCCOCCOCCOCCO* ; *CCOCCOCCOCC(=O)NC(COCCC(=O)NCCOCCOCCOCCO*)COCCC(=O)NCCOCCOCCOCCO* ; *OCCOCCOCCOCCNC(=O)CCOCC(COCCC(=O)NCCOCCOCCOCCO*)NC(=O)CCCCCn1cc(CCCC(=O)NCCOCCOCCOCCOCCC(*)=O)nn1]

In certain embodiments, Linker^{D}-(Linker^{A}) is selected from

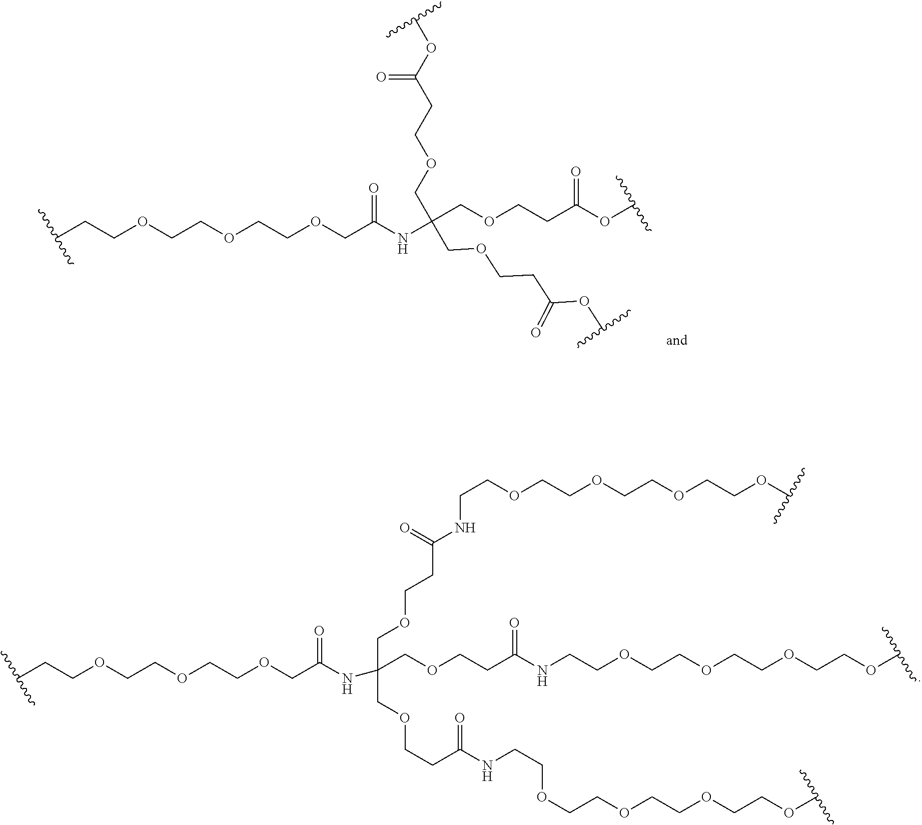 [SMILES: *CCOCCOCCOCC(=O)NC(COCCC(=O)O*)(COCCC(=O)O*)COCCC(=O)O* ; *CCOCCOCCOCC(=O)NC(COCCC(=O)NCCOCCOCCOCCO*)(COCCC(=O)NCCOCCOCCOCCO*)COCCC(=O)NCCOCCOCCOCCO*]

### II. COMPOUND TERMINOLOGY

Compounds are described using standard nomenclature. Unless defined otherwise, all technical and scientific terms used herein have the same meaning as is commonly understood by one of skill in the art to which this invention belongs.

The compounds in any of the Formulas described herein include as separate embodiments enantiomers, diastereomers, tautomers, racemates, rotamers or mixtures thereof, as if each is specifically described, unless otherwise indicated or otherwise excluded by context.

The terms “a” and “an” do not denote a limitation of quantity, but rather denote the presence of at least one of the referenced item. The term “or” means “and/or”. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of examples, or exemplary language (e.g., “such as”), is intended merely to better illustrate the invention and does not pose a limitation on the scope of the invention unless otherwise claimed. Unless defined otherwise, technical and scientific terms used herein have the same meaning as is commonly understood by one of skill in the art to which this invention belongs.

The present invention includes compounds with at least one desired isotopic substitution of an atom, at an amount above the natural abundance of the isotope, i.e., enriched.

Examples of isotopes that can be incorporated into compounds of the invention include isotopes of hydrogen, carbon, nitrogen, oxygen, phosphorous, fluorine, and chlorine, such as ²H, ³H, ¹¹C, ¹³C, ¹⁴C, ¹⁵N, ¹⁷O, ¹⁸O, ¹⁸F ³¹P, ³²P, ³⁵S, ³⁶Cl, and ¹²⁵¹respectively. In certain embodiments, isotopically labelled compounds can be used in metabolic studies (with, for example ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. For example, a ¹⁸F labeled compound may be desirable for PET or SPECT studies. Isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent.

By way of general example and without limitation, isotopes of hydrogen, for example, deuterium (²H) and tritium (³H) may optionally be used anywhere in described structures that achieves the desired result. Alternatively, or in addition, isotopes of carbon, e.g., ¹³C and ¹⁴C, may be used. In certain embodiments, the isotopic substitution is replacing hydrogen with a deuterium at one or more locations on the molecule to improve the performance of the drug, for example, the pharmacodynamics, pharmacokinetics, biodistribution, half-life, stability, AUC, Tmax, Cmax, etc. For example, the deuterium can be bound to carbon in a location of bond breakage during metabolism (an α-deuterium kinetic isotope effect) or next to or near the site of bond breakage (a β-deuterium kinetic isotope effect).

Isotopic substitutions, for example deuterium substitutions, can be partial or complete. Partial deuterium substitution means that at least one hydrogen is substituted with deuterium. In certain embodiments, the isotope is 80, 85, 90, 95 or 99% or more enriched in an isotope at any location of interest. In certain embodiments deuterium is 80, 85, 90, 95 or 99% enriched at a desired location. Unless otherwise stated, the enrichment at any point is above natural abundance, and in an embodiment is enough to alter a detectable property of the drug in a human.

In certain embodiments, the substitution of a hydrogen atom for a deuterium atom occurs within any variable group. For example, when any variable group is, or contain for example through substitution, methyl, ethyl, or methoxy, the alkyl residue may be deuterated (in nonlimiting embodiments, CDH₂, CD₂H, CD₃, CD₂CD₃, CHDCH₂D, CH₂CD₃, CHDCHD₂, OCDH₂, OCD₂H, or OCD₃ etc.). In certain other embodiments, a variable group has a “‘” or an “a” designation, which in certain embodiments can be deuterated. In certain other embodiments, when two substituents of the central core ring are combined to form a cyclopropyl ring, the unsubstituted methylene carbon may be deuterated.

The compound of the present invention may form a solvate with solvents (including water). Therefore, in certain embodiments, the invention includes a solvated form of the active compound. The term “solvate” refers to a molecular complex of a compound of the present invention (including a salt thereof) with one or more solvent molecules. Nonlimiting examples of solvents are water, ethanol, dimethyl sulfoxide, acetone and other common organic solvents. The term “hydrate” refers to a molecular complex comprising a compound of the invention and water. Pharmaceutically acceptable solvates in accordance with the invention include those wherein the solvent of crystallization may be isotopically substituted, e.g. D₂O, d₆-acetone, d₆-DMSO. A solvate can be in a liquid or solid form.

A dash (“-”) that is not between two letters or symbols is used to indicate a point of attachment for a substituent. For example, —(C═O)NH₂ is attached through carbon of the keto (C═O) group.

The term “substituted”, as used herein, means that any one or more hydrogens on the designated atom or group is replaced with a moiety selected from the indicated group, provided that the designated atom's normal valence is not exceeded and the resulting compound is stable. For example, when the substituent is oxo (i.e., —O) then two hydrogens on the atom are replaced. For example a pyridyl group substituted by oxo is a pyridone. Combinations of substituents and/or variables are permissible only if such combinations result in stable compounds or useful synthetic intermediates.

“Alkyl” is a branched, straight chain, or cyclic saturated aliphatic hydrocarbon group. In certain embodiments, the alkyl contains from 1 to about 12 carbon atoms, more generally from 1 to about 6 carbon atoms, from 1 to about 4 carbon atoms, or from 1 to 3 carbon atoms. In certain embodiments, the alkyl contains from 1 to about 8 carbon atoms. In certain embodiments, the alkyl is C₁-C₂, C₁-C₃, C₁-C₄, C₁-C₅ or C₁-C₆. The specified ranges as used herein indicate an alkyl group which is considered to explicitly disclose as individual species each member of the range described as a unique species. For example, the term C₁-C₆ alkyl as used herein indicates a straight or branched alkyl group having from 1, 2, 3, 4, 5, or 6 carbon atoms and also a carbocyclic alkyl group of 3, 4, 5, or 6 carbon atoms and is intended to mean that each of these is described as an independent species. For example, the term C₁-C₄alkyl as used herein indicates a straight or branched alkyl group having from 1, 2, 3, or 4 carbon atoms and is intended to mean that each of these is described as an independent species. When C₀-Cₙ alkyl is used herein in conjunction with another group, for example, (C₃-C₇cycloalkyl)C₀-C₄ alkyl, or —C₀-C₄alkyl(C₃-C₇cycloalkyl), the indicated group, in this case cycloalkyl, is either directly bound by a single covalent bond (C₀alkyl), or attached by an alkyl chain in this case 1, 2, 3, or 4 carbon atoms. Alkyls can also be attached via other groups such as heteroatoms as in —O—C₀-C₄alkyl(C₃-C₇cycloalkyl). Examples of alkyl include, but are not limited to, methyl, ethyl, n-propyl, isopropyl, n-butyl, isobutyl, sec-butyl, t-butyl, n-pentyl, isopentyl, tert-pentyl, neopentyl, n-hexyl, 2-methylpentane, 3-methylpentane, 2,2-dimethylbutane, 2,3-dimethylbutane, and hexyl.

When a term is used that includes “alk” it should be understood that “cycloalkyl” or “carbocyclic” can be considered part of the definition, unless unambiguously excluded by the context. For example and without limitation, the terms alkyl, alkenyl, alkynyl, alkoxy, alkanoyl, alkenloxy, haloalkyl, etc. can all be considered to include the cyclic forms of alkyl, unless unambiguously excluded by context.

“Alkenyl” is a branched or straight chain aliphatic hydrocarbon group having one or more carbon-carbon double bonds that may occur at a stable point along the chain. Nonlimiting examples are C₂-C₅alkenyl, C₂-C₇alkenyl, C₂-C₅alkenyl, C₂-C₅alkenyl and C₂-C₄alkenyl. The specified ranges as used herein indicate an alkenyl group having each member of the range described as an independent species, as described above for the alkyl moiety. Examples of alkenyl include, but are not limited to, ethenyl and propenyl.

“Alkynyl” is a branched or straight chain aliphatic hydrocarbon group having one or more carbon-carbon triple bonds that may occur at any stable point along the chain, for example, C₂-C₈alkynyl or C₂-C₆alkynyl. The specified ranges as used herein indicate an alkynyl group having each member of the range described as an independent species, as described above for the alkyl moiety. Examples of alkynyl include, but are not limited to, ethynyl, propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl and 5-hexynyl.

“Alkoxy” is an alkyl group as defined above covalently bound through an oxygen bridge (—O—). Examples of alkoxy include, but are not limited to, methoxy, ethoxy, n-propoxy, i-propoxy, n-butoxy, 2-butoxy, t-butoxy, n-pentoxy, 2-pentoxy, 3-pentoxy, isopentoxy, neopentoxy, n-hexoxy, 2-hexoxy, 3-hexoxy, and 3-methylpentoxy. Similarly an “alkylthio” or a “thioalkyl” group is an alkyl group as defined above with the indicated number of carbon atoms covalently bound through a sulfur bridge (—S—). In certain embodiments, the alkoxy group is optionally substituted as described above.

“Haloalkyl” indicates both branched and straight-chain alkyl groups substituted with 1 or more halogen atoms, up to the maximum allowable number of halogen atoms. Examples of haloalkyl include, but are not limited to, trifluoromethyl, monofluoromethyl, difluoromethyl, 2-fluoroethyl, and penta-fluoroethyl.

“Aryl” indicates an aromatic group containing only carbon in the aromatic ring or rings. In certain embodiments, the aryl group contains 1 to 3 separate or fused rings and is 6 to 14 or 18 ring atoms, without heteroatoms as ring members. The term “aryl” includes groups where a saturated or partially unsaturated carbocycle group is fused with an aromatic ring. The term “aryl” also includes groups where a saturated or partially unsaturated heterocycle group is fused with an aromatic ring so long as the attachment point is the aromatic ring. Such compounds may include aryl rings fused to a 4 to 7 or a 5 to 7-membered saturated or partially unsaturated cyclic group that optionally contains 1, 2 or 3 heteroatoms independently selected from N, O, B, P, Si and S, to form, for example, a 3,4-methylenedioxyphenyl group. Aryl groups include, for example, phenyl and naphthyl, including 1-naphthyl and 2-naphthyl. In certain embodiments, aryl groups are pendant. An example of a pendant ring is a phenyl group substituted with a phenyl group.

The term “heterocycle” refers to saturated and partially saturated heteroatom-containing ring radicals, where the heteroatoms may be selected from N, S, and O. The term “heterocycle” includes monocyclic 3-12 membered rings, as well as bicyclic 5-16 membered ring systems (which can include fused, bridged, or spiro, bicyclic ring systems). It does not include rings containing —O—O— or —S—S—portions. Examples of saturated heterocycle groups include saturated 4- to 7-membered monocyclic groups containing 1 to 4 nitrogen atoms [e.g., pyrrolidinyl, imidazolidinyl, piperidinyl, pyrrolinyl, azetidinyl, piperazinyl, and pyrazolidinyl]; saturated 4 to 6-membered monocyclic groups containing 1 to 2 oxygen atoms and 1 to 3 nitrogen atoms [e.g., morpholinyl]; saturated 3 to 6-membered heteromonocyclic group containing 1 to 2 sulfur atoms and 1 to 3 nitrogen atoms [e.g., thiazolidinyl]. Examples of partially saturated heterocycle radicals include but are not limited to, dihydrothienyl, dihydropyranyl, dihydrofuryl, and dihydrothiazolyl. Examples of partially saturated and saturated heterocycle groups include but are not limited to, pyrrolidinyl, imidazolidinyl, piperidinyl, pyrrolinyl, pyrazolidinyl, piperazinyl, morpholinyl, tetrahydropyranyl, thiazolidinyl, dihydrothienyl, 2,3-dihydro-benzo[1,4]dioxanyl, indolinyl, isoindolinyl, dihydrobenzothienyl, dihydrobenzofuryl, isochromanyl, chromanyl, 1,2-dihydroquinolyl, 1,2,3,4-tetrahydro-isoquinolyl, 1,2,3,4-tetrahydro-quinolyl, 2,3,4,4a,9,9a-hexahydro-1H-3-aza-fluorenyl, 5,6,7-trihydro-1,2,4-triazolo[3,4-a]isoquinolyl, 3,4-dihydro-2H-benzo[1,4]oxazinyl, benzo[1,4]dioxanyl, 2,3-dihydro-1H-1λ′-benzo[d]isothiazol-6-yl, dihydropyranyl, dihydrofuryl and dihydrothiazolyl. “Bicyclic heterocycle” includes groups wherein the heterocyclic radical is fused with an aryl radical wherein the point of attachment is the heterocycle ring. “Bicyclic heterocycle” also includes heterocyclic radicals that are fused or bridged with a carbocycle radical. For example partially unsaturated condensed heterocyclic group containing 1 to 5 nitrogen atoms, for example, indoline, isoindoline, partially unsaturated condensed heterocyclic group containing 1 to 2 oxygen atoms and 1 to 3 nitrogen atoms, partially unsaturated condensed heterocyclic group containing 1 to 2 sulfur atoms and 1 to 3 nitrogen atoms, and saturated condensed heterocyclic group containing 1 to 2 oxygen or sulfur atoms.

Non-limiting examples of bicyclic heterocycles include:

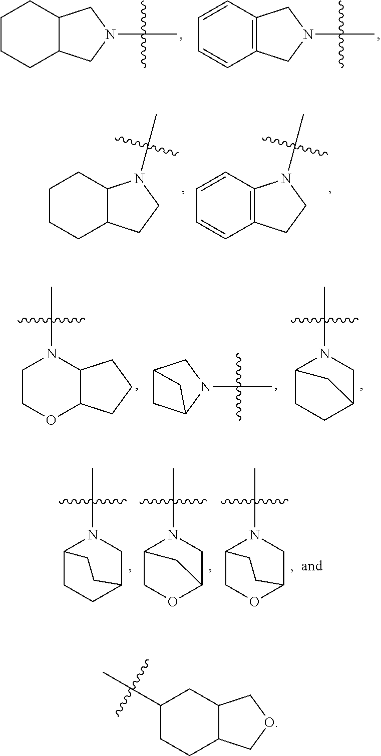

Unless otherwise drawn or clear from the context, the term “bicyclic heterocycle” includes cis and trans diastereomers. Non-limiting examples of chiral bicyclic heterocycles include:

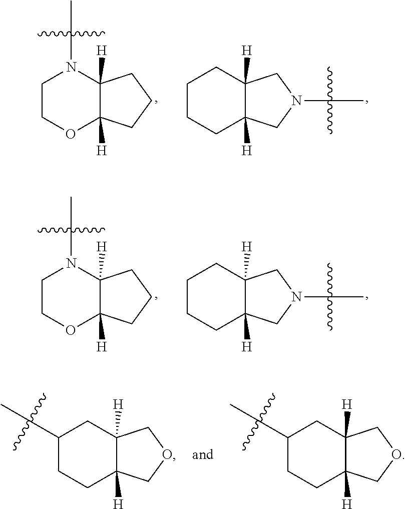

In certain alternative embodiments the term “heterocycle” refers to saturated and partially saturated heteroatom-containing ring radicals, where the heteroatoms may be selected from N, S, O, B, Si, and P.

“Heteroaryl” refers to a stable monocyclic, bicyclic, or multicyclic aromatic ring which contains from 1 to 5, or in some embodiments from 1, 2, or 3 heteroatoms selected from N, O, S, B, and P (and typically selected from N, O, and S) with remaining ring atoms being carbon, or a stable bicyclic or tricyclic system containing at least one 5, 6, or 7 membered aromatic ring which contains from 1 to 5, or in some embodiments from 1 to 2, heteroatoms selected from N, O, S, B or P with remaining ring atoms being carbon. In certain embodiments, the only heteroatom is nitrogen. In certain embodiments, the only heteroatom is oxygen. In certain embodiments, the only heteroatom is sulfur. Monocyclic heteroaryl groups typically have from 5 or 6 ring atoms. In some embodiments bicyclic heteroaryl groups are 8- to 10-membered heteroaryl groups, that is, groups containing 8 or 10 ring atoms in which one 5, 6, or 7-member aromatic ring is fused to a second aromatic or non-aromatic ring wherein the point of attachment is the aromatic ring. When the total number of S and O atoms in the heteroaryl group exceeds 1, these heteroatoms are not adjacent to one another. In certain embodiments, the total number of S and O atoms in the heteroaryl group is not more than 2. In another embodiment, the total number of S and O atoms in the aromatic heterocycle is not more than 1. Examples of heteroaryl groups include, but are not limited to, pyridinyl (including, for example, 2-hydroxypyridinyl), imidazolyl, imidazopyridinyl, pyrimidinyl (including, for example, 4-hydroxypyrimidinyl), pyrazolyl, triazolyl, pyrazinyl, furyl, thienyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, triazolyl, thiadiazolyl, thiadiazolyl, furazanyl, benzofurazanyl, benzothiophenyl, benzothiazolyl, benzoxazolyl, quinazolinyl, quinoxalinyl, naphthyridinyl, tetrahydrofuranyl, and furopyridinyl. Heteroaryl groups are optionally substituted independently with one or more substituents described herein. “Heteroaryloxy” is a heteroaryl group as described bound to the group it substituted via an oxygen, —O—, linker.

“Heteroarylalkyl” is an alkyl group as described herein substituted with a heteroaryl group as described herein.

“Arylalkyl” is an alkyl group as described herein substituted with an aryl group as described herein.

“Heterocycloalkyl” is an alkyl group as described herein substituted with a heterocyclo group as described herein.

When a compound moiety is “optionally substituted” it may be substituted as allowed by valence with one or more groups selected from alkyl (including C₁-C₄alkyl), alkenyl (including C₂-C₄alkenyl), alkynyl (including C₂-C₄alkynyl), haloalkyl (including C₁-C₄haloalkyl), —OR⁶, F, Cl, Br, I, —NR⁶R⁷, cyano, nitro, C(O)R³,

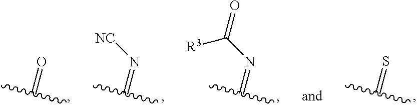

wherein the optional substituent is selected such that a stable compound results. For example

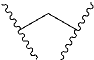 [SMILES: *C*]

could be substituted with 1 or 2 groups independently selected from alkyl, alkenyl, alkynyl, haloalkyl, —OR⁶, F, Cl, Br, I, —NR⁶R⁷, cyano, nitro, C(O)R³ so long as a stable compound results but only one group selected from

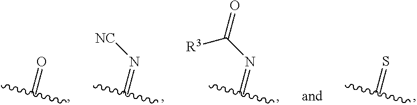

so long as a stable compound results.

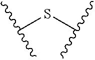 [SMILES: *S*]

on the other hand could only be substituted with 1 or 2 groups selected from

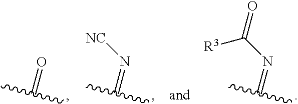

Non-limiting examples of optionally substituted CH₂ groups include:

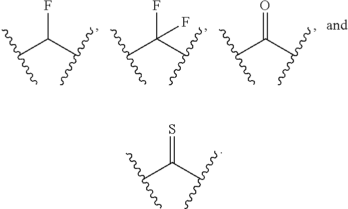

Non-limiting examples of optionally substituted —S— groups include:

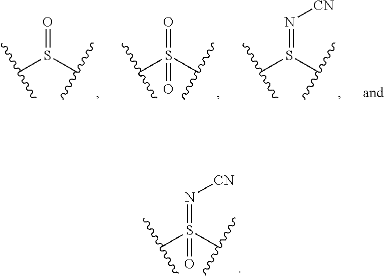

A “dosage form” means a unit of administration of an active agent. Examples of dosage forms include tablets, capsules, injections, suspensions, liquids, emulsions, implants, particles, spheres, creams, ointments, suppositories, inhalable forms, transdermal forms, buccal, sublingual, topical, gel, mucosal, subcutaneous, intramuscular, parenteral, systemic, intravenous, and the like. A “dosage form” can also include an implant for controlled delivery.

“Pharmaceutical compositions” are compositions comprising at least one active agent, and at least one other substance, such as a carrier. The present invention includes pharmaceutical compositions of the described compounds.

“Pharmaceutical combinations” are combinations of at least two active agents which may be combined in a single dosage form or provided together in separate dosage forms with instructions that the active agents are to be used together to treat any disorder described herein.

A “pharmaceutically acceptable salt” is a derivative of the disclosed compound in which the parent compound is modified by making an inorganic or organic, pharmaceutically acceptable, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Salts of the present compounds further include solvates of the compounds and of the compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include salts which are acceptable for human consumption and the quaternary ammonium salts of the parent compound formed, for example, from inorganic or organic acids. Examples, of such salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)₁₋₄—COOH, and the like, or using a different acid that produces the same counterion. Lists of additional suitable salts may be found, e.g., in Remington's Pharmaceutical Sciences, 17th ed., Mack Publishing Company, Easton, Pa., p. 1418 (1985).

The term “carrier” applied to pharmaceutical compositions/combinations of the invention refers to a diluent, excipient, or vehicle with which an active compound is provided.

A “pharmaceutically acceptable excipient” means an excipient that is useful in preparing a pharmaceutical composition/combination that is generally safe, acceptable for human consumption, and neither biologically nor otherwise inappropriate for administration to a host, typically a human. In certain embodiments, an excipient is used that is acceptable for veterinary use.

A “patient” or “host” or “subject” is a human or non-human animal in need of treatment or prevention of any of the disorders as specifically described herein. Typically, the host is a human. A “patient” or “host” or “subject” also refers to for example, a mammal, primate (e.g., human), cow, sheep, goat, horse, dog, cat, rabbit, rat, mice, bird and the like.

A “therapeutically effective amount” of a compound, pharmaceutical composition, or combination of this invention means an amount effective, when administered to a host, that provides a therapeutic benefit such as an amelioration of symptoms or reduction or diminution of the disease itself. In another aspect, a preventative amount can be administered that prevents or minimizes the risk of the disease mediated by the Extracellular Target Protein.

### Embodiments of “Alkyl”

In certain embodiments “alkyl” is a C₁-C₁₀alkyl, C₁-C₉alkyl, C₁-C₈alkyl, C₁-C₇alkyl, C₁-C₆alkyl, C₁-C₈alkyl, C₁-C₄alkyl, C₁-C₃alkyl, or C₁-C₇alkyl.

In certain embodiments “alkyl” has one carbon.

In certain embodiments “alkyl” has two carbons.

In certain embodiments “alkyl” has three carbons.

In certain embodiments “alkyl” has four carbons.

In certain embodiments “alkyl” has five carbons.

In certain embodiments “alkyl” has six carbons.

Non-limiting examples of “alkyl” include: methyl, ethyl, propyl, butyl, pentyl, and hexyl.

Additional non-limiting examples of “alkyl” include: isopropyl, isobutyl, isopentyl, and isohexyl.

Additional non-limiting examples of “alkyl” include: sec-butyl, sec-pentyl, and sec-hexyl.

Additional non-limiting examples of “alkyl” include: tert-butyl, tert-pentyl, and tert-hexyl.

Additional non-limiting examples of “alkyl” include: neopentyl, 3-pentyl, and active pentyl.

In an alternative embodiment the “alkyl” group is optionally substituted.

In an alternative embodiment the “alkenyl” group is optionally substituted.

In an alternative embodiment the “alkynyl” group is optionally substituted.

### Embodiments of “Haloalkyl”

In certain embodiments “haloalkyl” is a C₁-C₁₀haloalkyl, C₁-C₉haloalkyl, C₁-C₈haloalkyl, C₁-C₇haloalkyl, C₁-C₆haloalkyl, C₁-C₅haloalkyl, C₁-C₄haloalkyl, C₁-C₃haloalkyl, and C₁-C₂haloalkyl.

In certain embodiments “haloalkyl” has one carbon.

In certain embodiments “haloalkyl” has one carbon and one halogen.

In certain embodiments “haloalkyl” has one carbon and two halogens.

In certain embodiments “haloalkyl” has one carbon and three halogens.

In certain embodiments “haloalkyl” has two carbons.

In certain embodiments “haloalkyl” has three carbons.

In certain embodiments “haloalkyl” has four carbons.

In certain embodiments “haloalkyl” has five carbons.

In certain embodiments “haloalkyl” has six carbons.

Non-limiting examples of “haloalkyl” include:

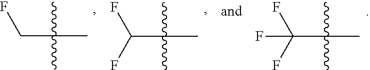

Additional non-limiting examples of “haloalkyl” include:

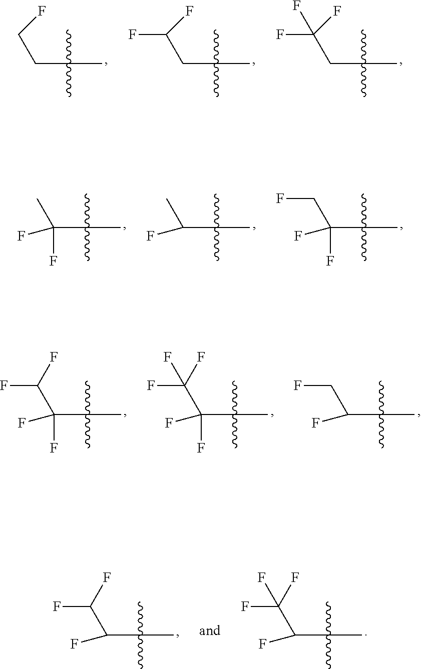

Additional non-limiting examples of “haloalkyl” include

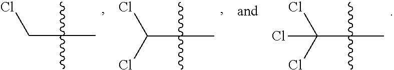

Additional non-limiting examples of “haloalkyl” include:

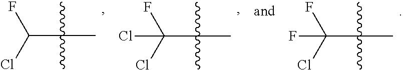

### Embodiments of “Heteroaryl”

Non-limiting examples of 5 membered “heteroaryl” groups include pyrrole, furan, thiophene, pyrazole, imidazole, triazole, isoxazole, oxazole, oxadiazole, oxatriazole, isothiazole, thiazole, thiadiazole, and thiatriazole.

Additional non-limiting examples of 5 membered “heteroaryl” groups include:

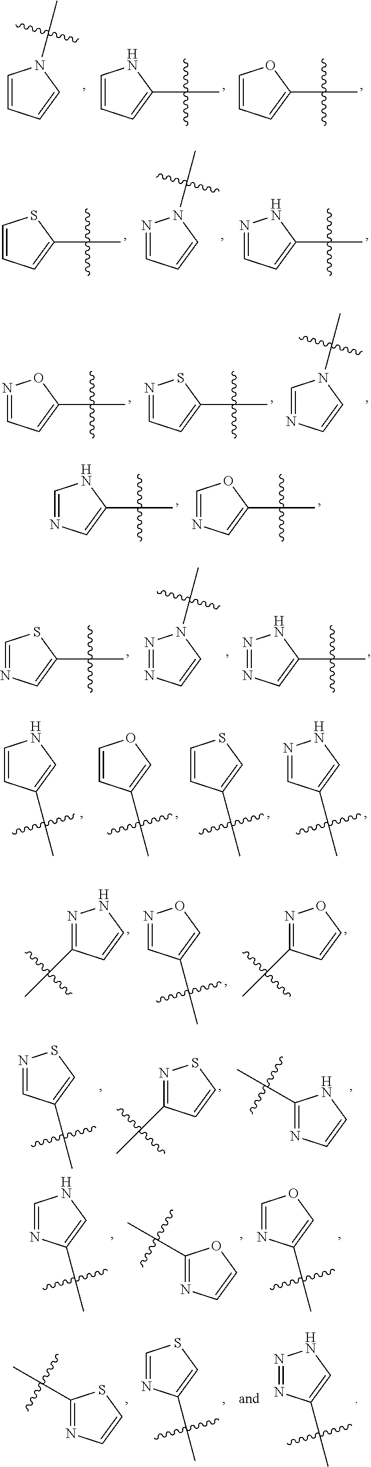

In certain embodiments “heteroaryl” is a 6 membered aromatic group containing 1, 2, or 3 nitrogen atoms (i.e. pyridinyl, pyridazinyl, triazinyl, pyrimidinyl, and pyrazinyl).

Non-limiting examples of 6 membered “heteroaryl” groups with 1 or 2 nitrogen atoms include:

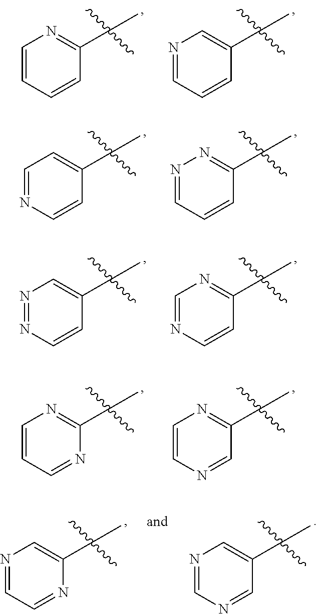

Additional non-limiting examples of heteroaryl groups include:

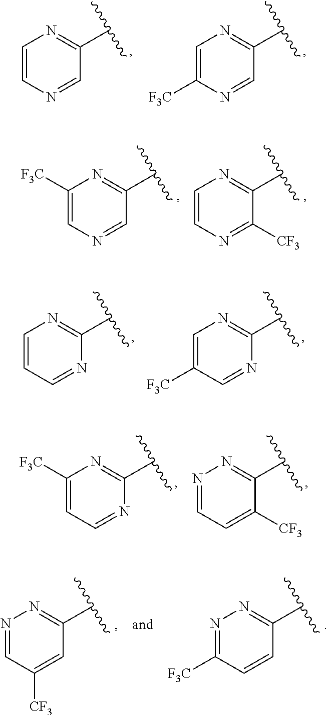

In certain embodiments “heteroaryl” is a 9 membered bicyclic aromatic group containing 1 or 2 atoms selected from nitrogen, oxygen, and sulfur.

Non-limiting examples of “heteroaryl” groups that are bicyclic include indole, benzofuran, isoindole, indazole, benzimidazole, azaindole, azaindazole, purine, isobenzofuran, benzothiophene, benzoisoxazole, benzoisothiazole, benzooxazole, and benzothiazole.

Additional non-limiting examples of “heteroaryl” groups that are bicyclic include:

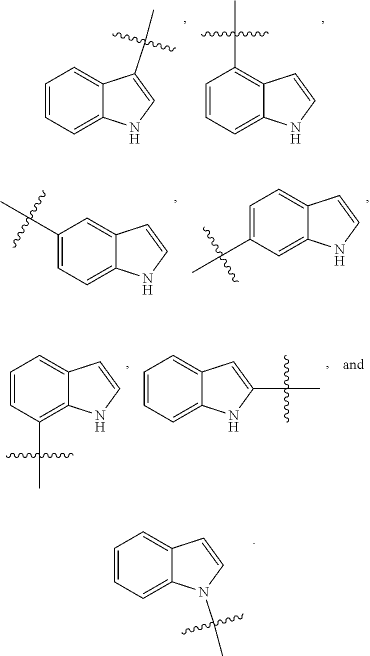

Additional non-limiting examples of “heteroaryl” groups that are bicyclic include:

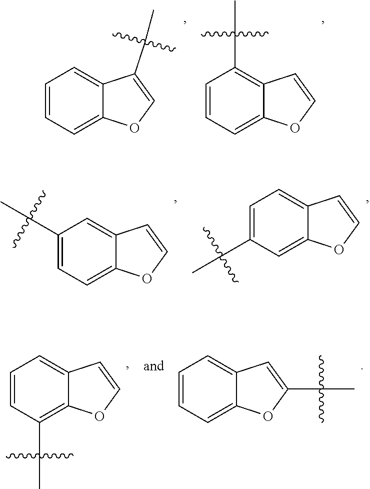 [SMILES: *c1coc2ccccc12 ; *c1cccc2occc12 ; *c1ccc2occc2c1 ; *c1ccc2ccoc2c1 ; *c1cccc2ccoc12 ; *c1cc2ccccc2o1]

Additional non-limiting examples of “heteroaryl” groups that are bicyclic include:

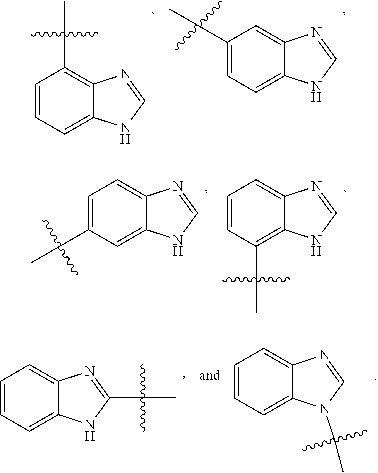

In certain embodiments “heteroaryl” is a 10 membered bicyclic aromatic group containing 1 or 2 atoms selected from nitrogen, oxygen, and sulfur.

Non-limiting examples of “heteroaryl” groups that are bicyclic include quinoline, isoquinoline, quinoxaline, phthalazine, quinazoline, cinnoline, and naphthyridine.

Additional non-limiting examples of “heteroaryl” groups that are bicyclic include:

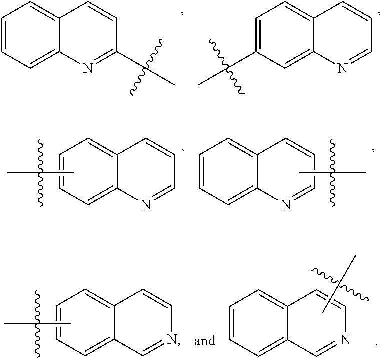

In certain embodiments “heteroaryl” refers to a stable monocyclic, bicyclic, or multicyclic aromatic ring which contains from 1 to 3, or in some embodiments from 1, 2, or 3 heteroatoms selected from N, O, S, B, and P (and typically selected from N, O, and S) with remaining ring atoms being carbon, or a stable bicyclic or tricyclic system containing at least one 5, 6, or 7 membered aromatic ring which contains from 1 to 3, or in some embodiments from 1 to 2, heteroatoms selected from N, O, S, B or P with remaining ring atoms being carbon.

### Embodiments of “Heterocycle”

In certain embodiments “heterocycle” refers to a cyclic ring with one nitrogen and 3, 4, 5, 6, 7, or 8 carbon atoms.

In certain embodiments “heterocycle” refers to a cyclic ring with one nitrogen and one oxygen and 3, 4, 5, 6, 7, or 8 carbon atoms.

In certain embodiments “heterocycle” refers to a cyclic ring with two nitrogens and 3, 4, 5, 6, 7, or 8 carbon atoms.

In certain embodiments “heterocycle” refers to a cyclic ring with one oxygen and 3, 4, 5, 6, 7, or 8 carbon atoms.

In certain embodiments “heterocycle” refers to a cyclic ring with one sulfur and 3, 4, 5, 6, 7, or 8 carbon atoms.

Non-limiting examples of “heterocycle” include aziridine, oxirane, thiirane, azetidine, 1,3-diazetidine, oxetane, and thietane.

Additional non-limiting examples of “heterocycle” include pyrrolidine, 3-pyrroline, 2-pyrroline, pyrazolidine, and imidazolidine.

Additional non-limiting examples of “heterocycle” include tetrahydrofuran, 1,3-dioxolane, tetrahydrothiophene, 1,2-oxathiolane, and 1,3-oxathiolane.

Additional non-limiting examples of “heterocycle” include piperidine, piperazine, tetrahydropyran, 1,4-dioxane, thiane, 1,3-dithiane, 1,4-dithiane, morpholine, and thiomorpholine.

Additional non-limiting examples of “heterocycle” include indoline, tetrahydroquinoline, tetrahydroisoquinoline, and dihydrobenzofuran wherein the point of attachment for each group is on the heterocyclic ring.

For example,

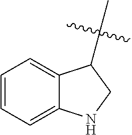 [SMILES: *C1CNc2ccccc21]

is a “heterocycle” group.

However,

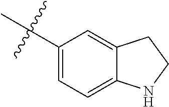 [SMILES: *c1ccc2c(c1)CCN2]

is an “aryl” group.

Non-limiting examples of “heterocycle” also include:

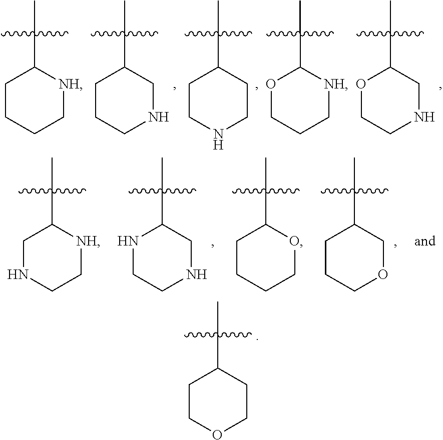

Additional non-limiting examples of “heterocycle” include:

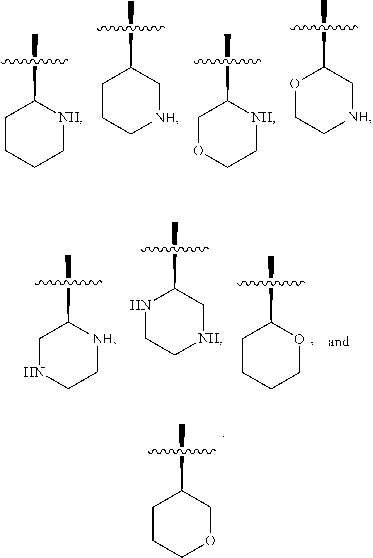

Additional non-limiting examples of “heterocycle” include:

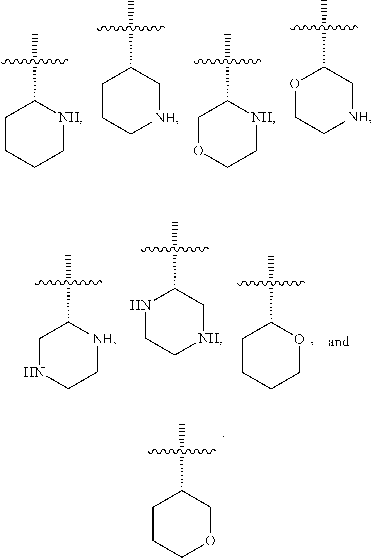

Non-limiting examples of “heterocycle” also include:

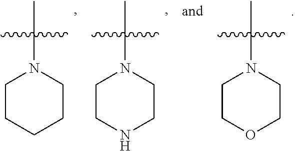

Non-limiting examples of “heterocycle” also include:

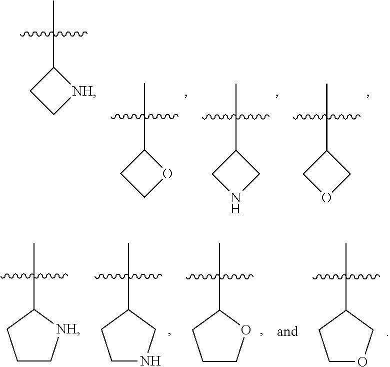

Additional non-limiting examples of “heterocycle” include:

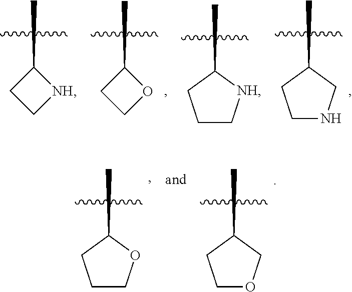

Additional non-limiting examples of “heterocycle” include:

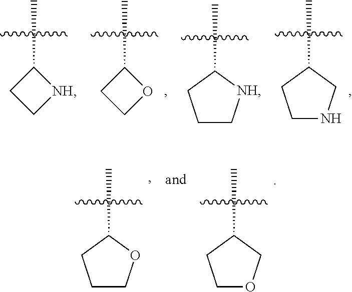

### Aryl

In certain embodiments “aryl” is a 6 carbon aromatic group (phenyl).

In certain embodiments “aryl” is a 10 carbon aromatic group (naphthyl).

In certain embodiments “aryl” is a 6 carbon aromatic group fused to a heterocycle wherein the point of attachment is the aryl ring. Non-limiting examples of “aryl” include indoline, tetrahydroquinoline, tetrahydroisoquinoline, and dihydrobenzofuran wherein the point of attachment for each group is on the aromatic ring.

For example

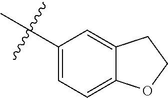 [SMILES: *c1ccc2c(c1)CCO2]

is an “aryl” group.

However,

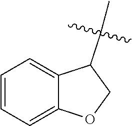 [SMILES: *C1COc2ccccc21]

is a “heterocycle” group.

### Embodiments of “Arylalkyl”

Non-limiting examples of “arylalkyl” include:

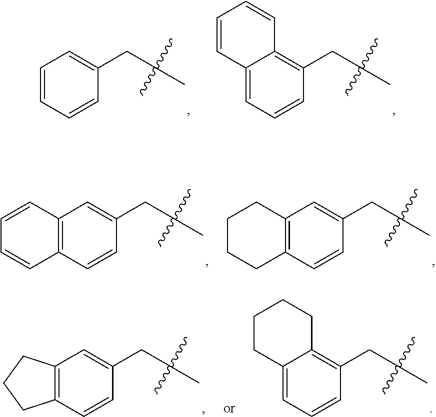

In certain embodiments “arylalkyl” is

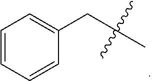 [SMILES: *Cc1ccccc1]

In certain embodiments the “arylalkyl” refers to a 2 carbon alkyl group substituted with an aryl group.

Non-limiting examples of “arylalkyl” include:

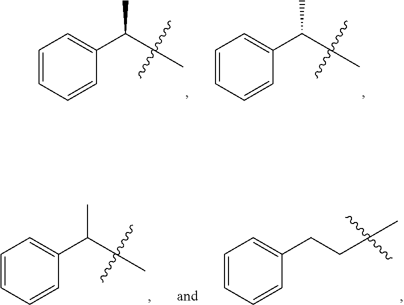

### III. Extracellular Protein Degradation

A wide range of well-known and characterized extracellular proteins can cause, modulate, or amplify diseases in vivo, such as abnormal cellular proliferation such as tumors and cancer, autoimmune disorders, inflammation and aging-related diseases. For example, extracellular proteins such as growth factors, cytokines, and chemokines bind to cell surface receptors, often initiate aberrant signaling in multiple diseases such as cancer and inflammation.

An Extracellular Protein Degrader described herein or its pharmaceutically acceptable salt and/or its pharmaceutically acceptable compositions can be used to treat a disorder which is mediated by the Target Extracellular Protein that binds to the Extracellular Protein Targeting Ligand. The described degraders are capable of targeting specific Extracellular Proteins that mediate pathological disorders for lysosomal degradation. The Target Extracellular Protein may modulate a disorder in a human via a mechanism of action such as modification of a biological pathway, pathogenic signaling, or modulation of a signal cascade or cellular entry. In certain embodiments, the Target Extracellular Protein is a protein that is not druggable in the classic sense in that it does not have a binding pocket or an active site that can be inhibited or otherwise bound, and cannot be easily allosterically controlled. In certain embodiments, the Target Extracellular Protein is a protein that is druggable in the classic sense, yet for therapeutic purposes, degradation of the protein is preferred to inhibition. The Target Extracellular Protein is recruited with an Extracellular Protein Targeting Ligand, which is a ligand for the Target Extracellular Protein. Typically, the Extracellular Protein Targeting Ligand binds the Target Extracellular Protein in a non-covalent fashion. In certain embodiments, the Target Extracellular Protein is covalently bound to the Extracellular Protein Targeting Ligand in a covalent manner that can be irreversible or reversible.

In certain embodiments, the Target Extracellular Protein is an extracellular protein which is not bound to the cell membrane. In certain embodiments, the Target Extracellular Protein is membrane bound with an extracellular domain. In other embodiments, the Target Extracellular Protein is a membrane protein, for example a transmembrane protein.

Accordingly, in some embodiments, a method to treat a host with a disorder mediated by a Target Extracellular Protein is provided that includes administering an effective amount of a degrader targeting the Target Extracellular Protein to the host, typically a human, optionally in a pharmaceutically acceptable composition.

The Target Extracellular Protein can be any amino acid sequence to which the degrader comprising an Extracellular Protein Targeting Ligand can be bound which through degradation thereof, results in a beneficial therapeutic effect. In certain embodiments, the Target Extracellular Protein is a non-endogenous peptide such as that from a pathogen or toxin. In another embodiment, the Target Extracellular Protein can be an endogenous protein that mediates a disorder. The endogenous protein can be either the normal form of the protein or an aberrant form. For example, the Target Extracellular Protein can be an extracellular mutant protein, or a protein, for example, where a partial, or full, gain-of-function or loss-of-function is encoded by nucleotide polymorphisms. In some embodiments, the degrader targets the aberrant form of the protein and not the normal form of the protein.

The Extracellular Protein Targeting Ligand is a ligand which covalently or non-covalently binds to a Target Extracellular Protein which has been selected for lysosomal degradation. In certain embodiments the Extracellular Protein Targeting Ligand is a small molecule or moiety (for example a peptide, nucleotide, antibody fragment, aptamer, biomolecule, or other chemical structure) that binds to a Target Extracellular Protein, and wherein the Target Extracellular Protein is a mediator of disease in a host as described in detail below. Exemplary Extracellular Protein Targeting Ligands are provided in the Figures.

### Anchor Bond

The Extracellular Protein Targeting Ligand (“EPTL”) is covalently bound to Linker in the protein degrader compound through the Anchor Bond (which is the chemical bond between the EPTL and either Linker^{B}, Linker^{C} or Linker^{D}). This bond can be placed at any location on the ligand that does not unacceptably disrupt the ability of the EPTL to bind to the Target Extracellular Protein. The Anchor Bond is depicted on the nonlimiting examples of Extracellular Protein Target Ligands in the figures as:

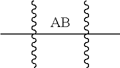 [SMILES: **]

A number of exemplary Target Extracellular Proteins for medical therapy described below have characterizing structural information in the well-known Protein Data Bank (“PDB”), which is a database for the three-dimensional structural information for large biological molecules such as proteins and nucleic acids. PDB includes x-ray crystallography and other information submitted by scientists around the world, and is freely accessible. See for example www.rcsb.org; www.wwpdb.org and www.uniprot.org. Using the PDB codes for example provided in Section ** or in the Data Bank itself, and technical references provided herein or otherwise publicly available, the skilled artisan can determine appropriate locations where the EPTL can be linked through an Anchor Bond to Linker^{B}, Linker^{C} or Linker^{D} to the receptor-binding moiety. For many of these proteins, published references describe how a range of ligands bind to the Target Extracellular Proteins, and from this information, one can determine reasonable Anchor Bond locations.

For example, the skilled artisan can use available visualization tools, including those available on the PDB website, to determine where the Extracellular Protein Targeting Ligand docks into to the Target Extracellular Protein. The skilled artisan can also import the crystal structure and the selected Extracellular Protein Targeting Ligand of interest into modeling software (including for example PyMOL, Glide, Maestro, RasMol, Visual Molecular Dynamics, Jmol, and AutoDock) to determine what portion of the Extracellular Protein Targeting Ligand is bound to the Target Extracellular Protein. The mannose 6-phosphate receptor ligand is then bound through the Linker and the Anchor Bond at a point that does not unduly adversely affect binding to the Target Extracellular Protein.

### Optional Substituents

In certain embodiments an Extracellular Protein Targeting Ligand described herein, for example in one of the figures or below is optionally substituted with 1, 2, 3, or 4 optional substituents independently selected from alkyl (including C₁-C₄alkyl), alkenyl (including C₂-C₄alkenyl), alkynyl (including C₂-C₄alkynyl), haloalkyl (including C₁-C₄haloalkyl), —OR⁶, F, Cl, Br, I, —NR⁶R⁷, cyano, nitro, C(O)R³,

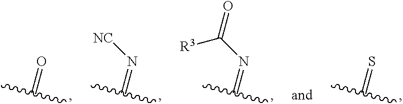

wherein the optional substituent is selected such that a stable compound results.

In certain embodiments the Target Extracellular Protein is selected from IgA, IgG, IgE, TNF-alpha, IL-1, IL-2, IL-6, IFN-γ, VEGF, TGF-β1, PCSK-9, CPB2, ChE, CCL2, Factor VII, Factor IX, CD40L, Factor Xa, Factor XI, Factor XIa, Factor XII, Factor XIII, FGF1, FGF2, FN1, IL-5, IL-8, IL-10, IL-21, IL-22, Kallikrein 1, LPL, MMP1, MIF, GIF, L-dopachrome isomerase, or phenylpyruvate tautomerase, neutrophil elastase, Prothrombin, KLKB1, PLG, PAI-1, endothelial plasminogen activator inhibitor, serpin E1, phospholipases A2, PLA2, PA21B, PLA2G1B, PLA2-IB, PLA2, PLAZA, PAZIIA, PLA2G2A, PLA2-IIA, PGF, plasminogen activator, tissue type (tPA, PLAT), Transforming growth factor beta 2 (TGF-β2, TGFB2), thrombospondin 1, Urokinase, Urokinase-type plasminogen activator, complement factor B, complement factor D, target complement factor H, and complement component 5.

In certain embodiments, where the Target Extracellular Protein has a receptor the Target Extracellular Protein can be used to degrade the receptor.

In certain embodiments the Extracellular Protein Targeting Ligand is a ligand for a protein selected from IgA, IgG, IgE, TNF-alpha, IL-1, IL-2, IL-6, IFN-γ, VEGF, TGF-β1, PCSK-9, CPB2, ChE, CCL2, Factor VII, Factor IX, CD40L, Factor Xa, Factor XI, Factor XIa, Factor XII, Factor XIII, FGF1, FGF2, FN1, IL-5, IL-8, IL-10, IL-21, IL-22, Kallikrein 1, LPL, MMP1, MIF, GIF, L-dopachrome isomerase, or phenylpyruvate tautomerase, neutrophil elastase, Prothrombin, KLKB1, PLG, PAI-1, endothelial plasminogen activator inhibitor, serpin E1, phospholipases A2, PLA2, PA21B, PLA2G1B, PLA2-IB, PLA2, PLAZA, PA21IA, PLA2G2A, PLA2-IIA, PGF, plasminogen activator, tissue type (tPA, PLAT), Transforming growth factor beta 2 (TGF-β2, TGFB2), thrombospondin 1, Urokinase, Urokinase-type plasminogen activator, complement factor B, complement factor D, target complement factor H, and complement component 5.

In other embodiments the Extracellular Protein Targeting Ligand is a ligand for anti-B1AR.

### Amino Acids

In certain embodiments the Extracellular Protein Targeting Ligand comprises one or more amino acids. The invention contemplates using natural amino acids, unnatural amino acids, or any combination thereof to achieve desired targeting ligand properties.

The term “natural amino acid” refers to an amino acid selected from alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine.

In certain embodiments a natural amino acid is replaced with a corresponding unnatural amino acid for example substituting a phenylalanine for a 4-chloro-phenylalanine. Non-limiting examples of unnatural amino acids include: 4-chloro-phenylalanine, 3-fluoro-phenalalanine, 4-trifluoromethyl-phaenylalanine, 3,4-dichloro-phenylalanine, 4-phenyl-phenylalanine, N-methylalanine, N-methylglutamic acid, N-methylphenylalanine, and homoserine.

Additional examples of non-natural amino acids include:

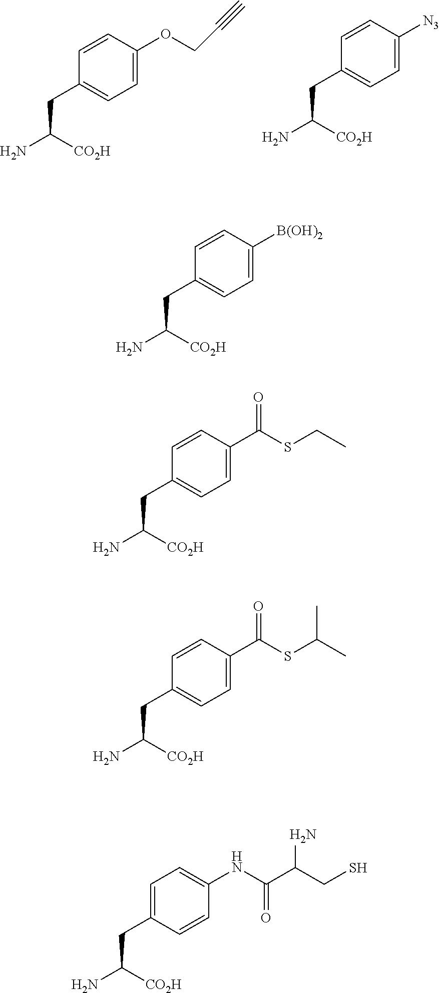 [SMILES: C#CCOc1ccc(C[C@H](N)C(=O)O)cc1 ; [N-]=[N+]=Nc1ccc(C[C@H](N)C(=O)O)cc1 ; N[C@@H](Cc1ccc(B(O)O)cc1)C(=O)O ; CCSC(=O)c1ccc(C[C@H](N)C(=O)O)cc1 ; CC(C)SC(=O)c1ccc(C[C@H](N)C(=O)O)cc1 ; NC(CS)C(=O)Nc1ccc(C[C@H](N)C(=O)O)cc1]

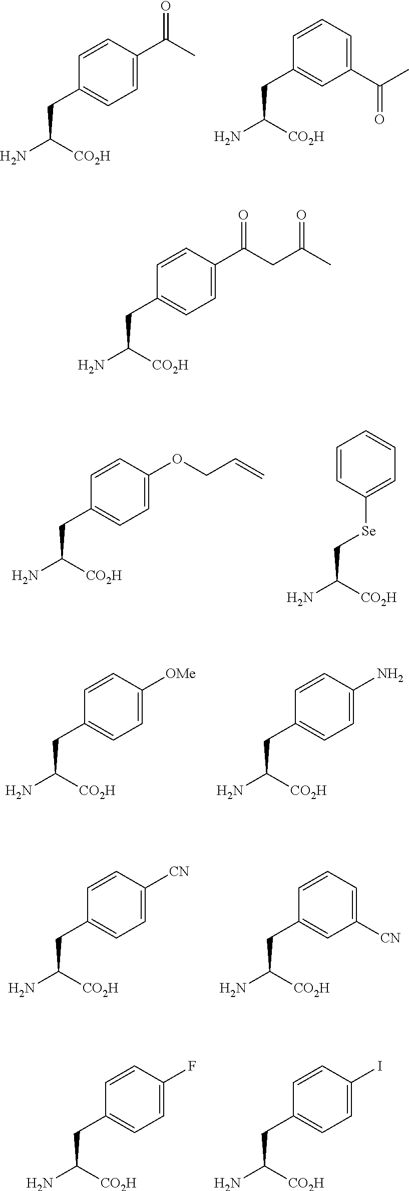

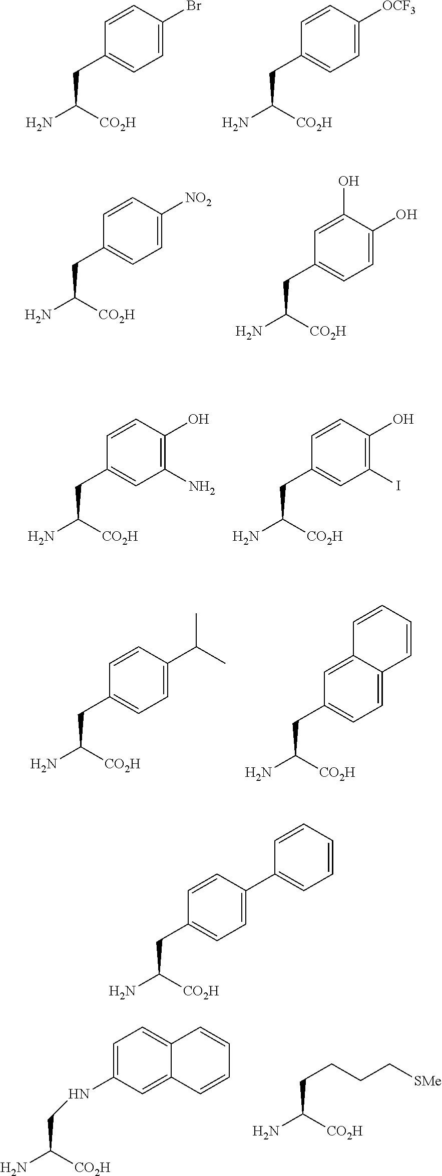

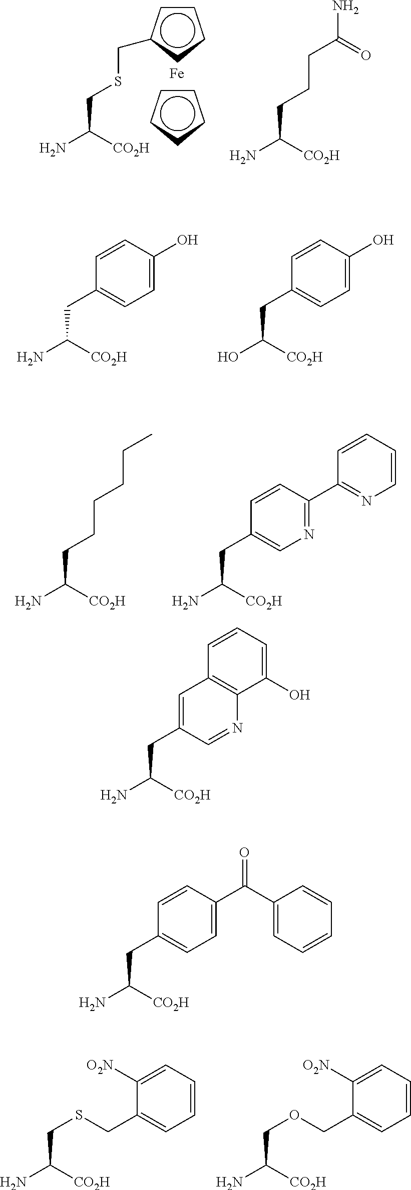

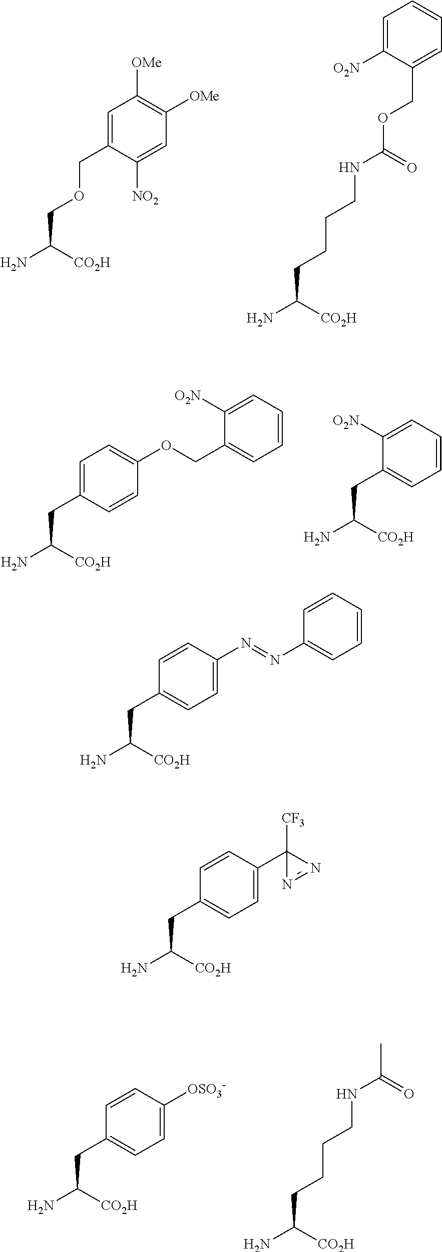

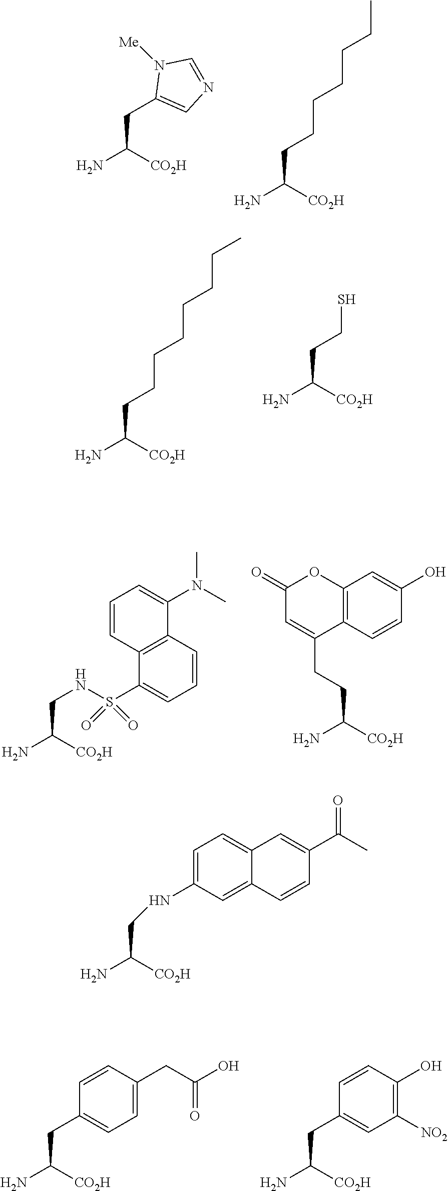

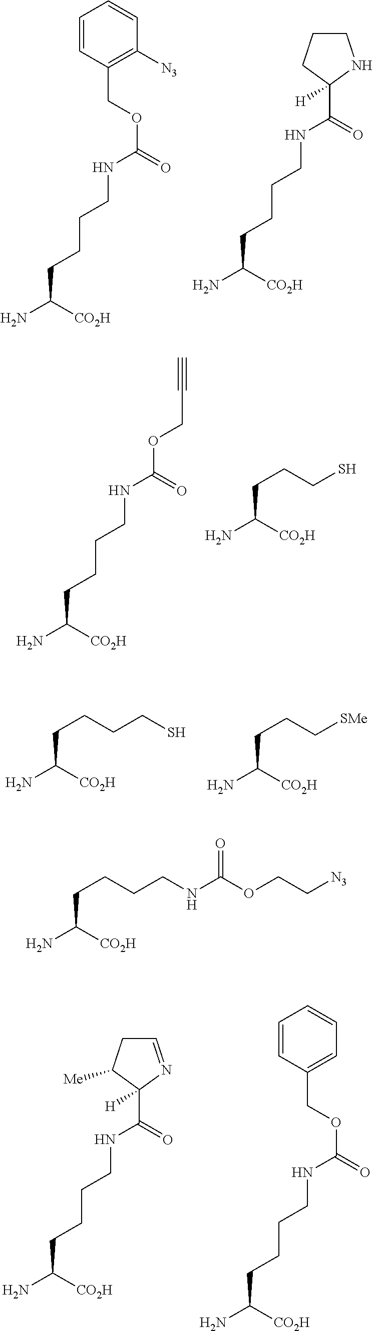

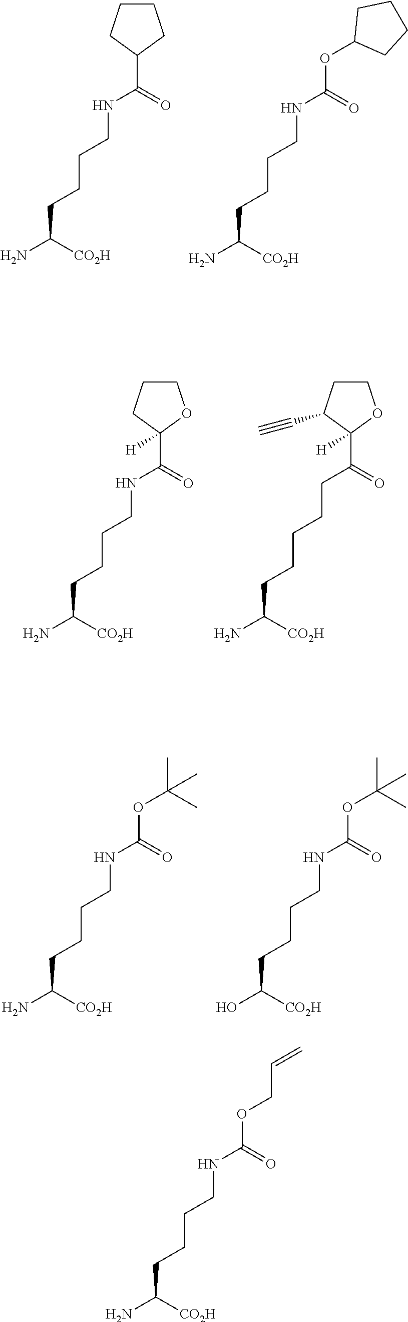

In certain embodiments the Extracellular Protein Targeting Ligand is a sequence of amino acids. In certain embodiments the amino acid sequence is connected to the Linker portion of the molecule by a bond to a terminal amine. In certain embodiments the amino acid sequence is connected to the Linker portion of the molecule by a bond to a terminal carboxylic acid (e.g. an ester or amide). In certain embodiments the peptide includes an amine, hydroxyl, or carboxylic acid side chain and the linker may be bound to one of these sidechains.

For example, when the amino acid sequence is SEQ ID NO: 1 MLKKIE non-limiting examples of locations wherein the peptide may be attached to the linker include:

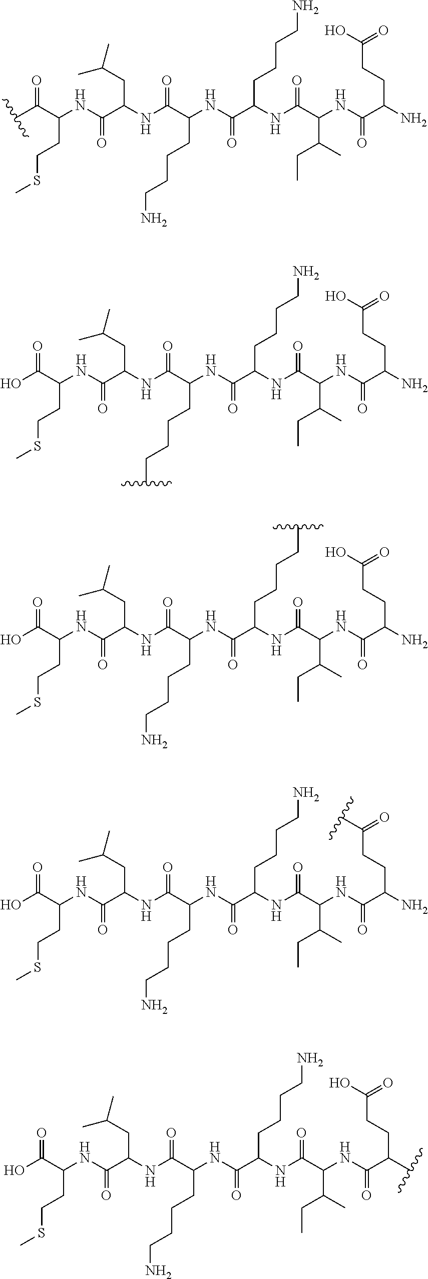 [SMILES: *C(=O)C(CCSC)NC(=O)C(CC(C)C)NC(=O)C(CCCCN)NC(=O)C(CCCCN)NC(=O)C(NC(=O)C(N)CCC(=O)O)C(C)CC ; *CCCCC(NC(=O)C(CCCCN)NC(=O)C(NC(=O)C(N)CCC(=O)O)C(C)CC)C(=O)NC(CC(C)C)C(=O)NC(CCSC)C(=O)O ; *CCCCC(NC(=O)C(NC(=O)C(N)CCC(=O)O)C(C)CC)C(=O)NC(CCCCN)C(=O)NC(CC(C)C)C(=O)NC(CCSC)C(=O)O ; *C(=O)CCC(N)C(=O)NC(C(=O)NC(CCCCN)C(=O)NC(CCCCN)C(=O)NC(CC(C)C)C(=O)NC(CCSC)C(=O)O)C(C)CC ; *C(CCC(=O)O)C(=O)NC(C(=O)NC(CCCCN)C(=O)NC(CCCCN)C(=O)NC(CC(C)C)C(=O)NC(CCSC)C(=O)O)C(C)CC]

The amino acid sequence can be attached to the Linker with chemistry described herein and as otherwise known in the art. For example, when the desired linking group is an amide the linker can be presented with an amine, carboxylic acid, ester or other amide precursor and the targeting ligand can be attached with an amide coupling reaction such as a HATU or HBTU coupling reaction.

Non-limiting examples of Extracellular Protein Targeting Ligands that are a sequence of amino acids include aptamers, antibodies, and peptides. In certain embodiments the left most amino acid listed in the sequence listing is the C-terminus. In other embodiments the right most amino acid listed in the sequence listing is the C-terminus.

In certain embodiments the amino acid sequence refers to a sequence without specified chirality. In other embodiments the amino acid sequence is all D-, all L-, or a mixture of D- and L-amino acids.

### IL-1

In some embodiments, the Target Extracellular Protein is human interleukin-1 (IL-1) (UniProtKB-P01584 (IL1B_HUMAN)). IL-1 is a potent proinflammatory cytokine. Initially discovered as the major endogenous pyrogen, induces prostaglandin synthesis, neutrophil influx and activation, T-cell activation and cytokine production, B-cell activation and antibody production, and fibroblast proliferation and collagen production. IL-1 promotes Th17 differentiation of T-cells, and Synergizes with IL12/interleukin-12 to induce IFNG synthesis from T-helper 1 (Th1) cells. IL-1 has been implicated in a number of auto-inflammatory and autoimmune disorders, including, but not limited to, Blau syndrome, cryopyrin-associated periodic syndromes, familial Mediterranean fever, Majeed syndrome; mevalonate kinase deficiency syndrome, pyogenic arthritis-pyoderma gangrenosum-acne syndrome, tumor necrosis factor receptor-associated periodic syndrome, Behçet's Disease, Sjogren's Syndrome, gout and chondrocalcinosis, periodic fever, aphthous stomatitis, pharyngitis, and cervical adenitis (or PFAPA) syndrome, rheumatoid arthritis, Type 2 diabetes mellitus, acute pericarditis, Chronic interstitial lung diseases (ILDs), Still's Disease,

The Protein Data Bank website provides the crystal structure of IL-1 searchable by 91LB (Yu, B., et al., Proc Natl Acad Sci USA, 1999, 96 103-108); 111B (Finzel, B. C., et al., J Mol Biol., 1989, 209 779-791); and 3040 (Wang et al., Nat. Immunol., 2010, 11:905-911); as well as the crystal structure of IL-1 bound to various compounds searchable by 4G6J (Blech, M., et al., J Mol Biol., 2013, 425 94-111); 5BVP (Rondeau e al., MAbs, 2015, 7 1151-1160); and 3LTQ (Barthelmes, K., et al., J Am Chem. Soc., 2011, 133 808-819). Additionally, Guy et al., provides insight into the crystal structure of a small antagonist peptide bound to interleukin-1 receptor type 1 (Guy et al., The Journal of Biological Chemistry, 2000, 275, 36927-36933).

Potential IL-1 direct or indirect inhibitors are described in FIG. 1. Additional IL-1 Targeting Ligands can be found in, for example, U.S. Pat. No. 9,694,015, each of which is incorporated herein by reference. Additional binding ligands include rilonacept or a binding fragment thereof (J Rheumatol. 2012; 39:720-727 (2012); and Canakinumab, or a binding fragment thereof (J Rheumatol. 2004; 31:1103-1111).

In certain embodiments the IL-1 Targeting Ligand is selected from

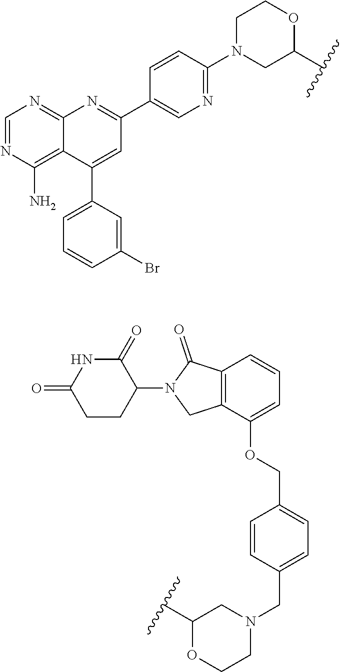 [SMILES: *C1CN(c2ccc(-c3cc(-c4cccc(Br)c4)c4c(N)ncnc4n3)cn2)CCO1 ; *C1CN(Cc2ccc(COc3cccc4c3CN(C3CCC(=O)NC3=O)C4=O)cc2)CCO1]

### IL-2

In some embodiments, the Target Extracellular Protein is human interleukin-2 (IL-2) (UniProtKB-P60568 (IL2_HUMAN)). IL-2 is a potent pro-inflammatory cytokine. IL-2 has been implicated in host versus graft rejection and other autoimmune disorders.

The Protein Data Bank website provides the crystal structure of IL-2 searchable by 1M4C and 1M47 (Arkin, M. R., et al., Proc.Natl.Acad.Sci.USA, 2003, 100:1603-1608); as well as the crystal structure of IL-2 bound to various compounds searchable by 4NEJ and 4NEM (Brenke, R., et al.); 1QVN (Thanos, C. D., et al., Proc Natl Acad Sci USA, 2006, 103 15422-15427); 1P W6 and 1PY2 (Thanos, C. D., et al., J Am Chem Soc., 2003, 125 15280-15281); 1NBP (Hyde, J., et al., Biochemistry, 2003, 42 6475-6483); and 1M48, 1M49, 1M4A, 1M4B, and 1M4C (Arkin, M. R., et al., Proc Natl Acad Sci USA, 2003, 100 1603-1608). Additionally, Stauber, D. J., et al, provides insight into the crystal structure of the IL-2 signaling complex: paradigm for a heterotrimeric cytokine receptor (Stauber, D. J., et al., PNAS, 2006, 103 (8), 2788-2793).

Representative IL-2 Targeting Ligands are provided in FIG. 1. Additional IL-2 Targeting Ligands can be found in, for example, U.S. Pat. Nos. 8,802,721; 9,682,976, 9,708,268; Eur J Med Chem 83:294-306 (2014), J Med Chem 60:6249-6272 (2017); Nature 450:1001-1009 (2007); each of which is incorporated by reference herein.

In certain embodiments the IL-2 Targeting Ligand is selected from

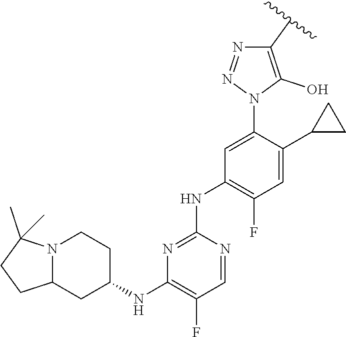 [SMILES: *c1nnn(-c2cc(Nc3ncc(F)c(N[C@H]4CCN5C(CCC5(C)C)C4)n3)c(F)cc2C2CC2)c1O]

### IL-6

In some embodiments, the Target Extracellular Protein is human inteleukin-6 (IL-6) (UniProtKB-P05231 (IL6_HUMAN)). IL-6 is a cytokine with a wide variety of biological functions. It is a potent inducer of the acute phase response and plays an essential role in the final differentiation of B-cells into Ig-secreting cells. It is also involved in lymphocyte and monocyte differentiation. It also acts on B-cells, T-cells, hepatocytes, hematopoietic progenitor cells and cells of the CNS, and is required for the generation of T (H) 17 cells. IL-6 has been implicated in a number of inflammatory diseases and cancers, including, but not limited to, Castleman's disease, metastatic castration-associated prostate cancer, renal cell carcinoma, large-cell lung carcinoma, ovarian cancer, rheumatoid arthritis, asthma.

The Protein Data Bank website provides the crystal structure of IL-6 searchable by 1P9M (Boulanger, M. J., et al., Science, 2003, 300:2101-2104); 1ALU (Somers et al., EMBO J., 1997, 16, 989-997); 11L6 and 21L6 (Xu, G. Y., et al., J Mol Biol., 1997, 268 468-481) and 1N26 (Varghese et al., Proc Natl Acad Sci USA., 2002, 99 15959-15964); as well as the crystal structure of IL-6 bound to various compounds searchable by 4CNI (Shaw, S., et al., Mabs, 2014, 6:773); and 4NI7 and 4NI9 (Gelinas et al., J Biol Chem. 2014, 289 (12), 8720-8734). Additionally, Gelinas et al., provides insight into the crystal structure of interleukin-6 in complex with a modified nucleic acid ligand (Gelinas, A. D., et al., J Biol Chem. 2014, 289 (12), 8720-8734); and Somers et al., provides insight into the crystal structure of interleukin 6: implications for a novel mode of receptor dimerization and signaling.

Potential IL-6 direct or indirect inhibitors are provided in FIG. 1. Additional potential IL-6 direct or indirect inhibitors can be found in, for example, U.S. Pat. Nos. 8,901,310; 10,189,796; 9,694,015; each incorporated herein by reference. In another embodiment the IL-6 Extracellular Targeting Ligand is AvimarC326 or a binding fragment thereof which is described in Nat Biotechnol 23, 1556-1561 (2005).

### IFN-γ

In some embodiments, the Target Extracellular Protein is human interferon-γ (IFN-γ) (UniProtKB-Q14609 (Q14609_HUMAN)). IFN-γ is a immunoregulatory cytokine. IFN-γ has been implicated in a number of autoimmune disorders, including, but not limited to rheumatoid arthritis, multiple sclerosis (MS), corneal transplant rejection, and various autoimmune skin diseases such as psoriasis, alopecia areata, vitiligo, acne vulgaris, and others.

The Protein Data Bank website provides the crystal structure of IFN-γ searchable by 1HIG (Ealick, S. E., et al., Science 252, 1991, 698-702); as well as the crystal structure of IFN-γ bound to various compounds searchable by 6E3K and 6E3L (Mendoza, J. L., et al., Nature, 2019, 567 56-60). Additionally, Randal et al., provides insight into the structure and activity of a monomeric interferon-γ: α-chain receptor signaling complex (Randal, M., et al., Structure, 2001, 9 (2), 155-163).

Representative IFN-γ Targeting Ligands are described in FIG. 1. Additional IFN-γ Targeting Ligands can be found in, for example, J Med Chem 57:4511-20 (2014); which is incorporated by reference herein.

### Vascular Epithelial Growth Factor (VEGF)

In some embodiments, the Target Extracellular Protein is human vascular epithelial growth factor (VEGF) (UniProtKB-P15692 (VEGFA_HUMAN)). VEGF is a growth factor active in angiogenesis, vasculogenesis, and endothelial cell growth. VEGF induces endothelial cell proliferation, promotes cell migration, inhibits apoptosis and induces permeabilization of blood vessels. VEGF has been implicated in the vascularization and angiogenesis of tumors.

The Protein Data Bank website provides the crystal structure of VEGF searchable by 3QTK (Mandal, K., et al., Angew Chem Int Ed Engl., 2011, 50 8029-8033); and 4KZN (Shen et al.); as well as the crystal structure of VEGF bound to various compounds searchable by 504E (Lobner, E., et al., MAbs, 2017, 9 1088-1104); 4QAF (Giese, T., et al.,); 5DN2 (Tsai, Y. C. I., et al., FEBS, 2017, J 283 1921-1934); 4GLS (Mandal, K., et al., Proc Natl Acad Sci USA, 2012, 109 14779-14784); and 1KMX (Stauffer, M. E. et al., J Biomol NMR, 2002, 23 57-61). Additionally, Mueller, Y. A., et al, provides insight into the Crystal structure and functional mapping of the kinase domain receptor binding site of VEGF (Mueller, Y. A., et al., Proc Natl Acad Sci USA., 1997 Jul. 8; 94 (14): 7192-7197).

Representative VEGF Targeting Ligands are provided in FIG. 1. Additional VEGF Targeting Ligands include, but are not limited to, the peptide (SEQ ID NO: 2) (Biochemistry 1998, 37, 17754-177764). In some embodiments, the VEGF Targeting Ligand is the peptide VEPNCDIHVMWEWECFERL (SEQ ID NO: 2), wherein Leu-19 is conjugated to an amide (—NH₂) group. Additional VEGF Targeting Ligands are provided in, for example, J Med Chem 57:3011-29 (2014), U.S. Pat. Nos. 9,884,843, 9,446,026, J Med Chem 53:1686-99 (2010), J Med Chem 48:8229-36 (2005), J Nat Prod 76:29-35 (2013), each of which is incorporated herein by reference. All references cited herein are incorporated by reference.

### Transforming Growth Factor —B1 (TGF-β1)

In some embodiments, the Target Extracellular Protein is human transforming growth factor-β1 (TGF-β1) (UniProtKB-P01137 (TGFB1_HUMAN)). TGF-β1 is a multifunctional protein that regulates the growth and differentiation of various cell types and is involved in various processes, such as normal development, immune function, microglia function and responses to neurodegeneration. TGF-β1 can promote either T-helper 17 cells (Th17) or regulatory T-cells (Treg) lineage differentiation in a concentration-dependent manner. TGF-β1 expression in the tumor microenvironment has been associated with a poor prognosis, and is implicated in TGF-β1 mediated tumor suppression via T-cell exclusion. TGF-β1 expression has also been implicated in hematological malignancies and fibrosis.

The Protein Data Bank website provides the crystal structure of TGF-β1 searchable by 5E8S, 5E8T, and 5E8U (Tebben, A. J., et al., Acta Crystallogr D Struct Biol., 2016, 72 658-674); 2L5S (Zuniga, J. E., et al, J Mol Biol., 2011, 412 601-618); and 2PJY (Groppe, J., et al., Mol Cell, 2008, 29 157-168); as well as the crystal structure of TGF-β1 bound to various compounds searchable by 5QIK, 5QIL and 5QIM, (Zhang, Y., et al., ACS Med Chem Lett., 2018, 9 1117-1122); 6B8Y (Harikrishnan, L. S., et al., Bioorg Med Chem., 2018, 26 1026-1034); 5E8W, 5E8X, 5E8Z, and 5E90 (Tebben, A. J., et al., Acta Crystallogr D Struct Biol., 2016, 72 658-674); 3TZM (Ogunjimi, A. A. et al., Cell Signal, 2012, 24 476-483); 2X70 (Roth, G. J., et al., J Med Chem., 2010, 53 7287); 3KCF (Guckian, K., et al., Bioorg Med Chem Lett., 2010, 20 326-329); 3FAA (Bonafoux, D., et al., Bioorg Med Chem Lett., 2009, 19 912-916); 1VJY (Gellibert, F, J., et al., J Med Chem., 2004 47 4494-4506); and 1PY5 (Sawyer, J. S., et al., Bioorg Med Chem Lett., 2004, 14 3581-3584). Additionally, Hinck et al., provides insight into the structural studies of the TGF-βs and their receptors and further insight into evolution of the TGF-β superfamily (Hinck, A., FEBS, 2012, 586 (14), 1860-1870).

Representative TGF-β1 Targeting Ligands are provided in FIG. 1. In some embodiments, the TGF-β1 Targeting Ligand is the peptide KRFK peptide (J. Biol. Chem. Vol. 274 (No. 19) pp. 13586-13593 (1999) (incorporated herein by reference). Additional TGF-β1 Targeting Ligands are provided in, for example, Bioorg Med Chem Lett 21:5642-5 (2011), which is incorporated herein by reference.

### FHR3

The human complement factor H-related protein 3 (FHR-3) belongs to the complement factor H (FH)-family. Factor H (FH), a major negative regulator of alternative complement pathway activation, belongs to a family that also includes five other related family members thought to have arisen from nonallelic homologous recombination and interlocus gene conversion including: complement factor H-related protein 1 (FHR1), complement factor H-related protein 2 (FHR2), complement factor H-related protein 3 (FHR3), complement factor H-related protein 4 with isoforms 4A and 4B (FHR4A and FHR4B) and complement factor H-related protein 5 (FHR5).

FHR3, unlike factor H, lacks the complement regulatory domains essential for complement inactivation and also competes with factor H, resulting in complement over-activation. Thus, the present invention provides compounds for use in modulating the concentration of complement factor H-proteins, specifically FHR3, to remove factor H's competitor and thereby restore factor H-mediated regulation to treat disorders caused by excessive complement activation.

Due to the central role that factor H plays in the regulation of complement, there are many clinical implications arising from aberrant FH activity. Loss of function mutation in factor H increase susceptibility to the renal diseases, atypical hemolytic uremic syndrome (aHUS) and dense deposit disease (ODD), whilst polymorphic variation of complement factor H has been strongly associated with important human diseases, including age-related macular degeneration (AMO) and meningococcal sepsis (Clin Exp Immunol 151 (2): 210-230; Immunobiology 217 (11): 1034-1046).

In certain embodiment, the invention provides the use in the treatment of a FHR3 mediated disease or disorder.

In certain embodiments, the FHR3 mediated disease or disorder is a complement-related diseases, disorders of complement dysregulation, autoimmune diseases, kidney disease, retinal degenerative diseases, Rheumatic Diseases, associated degenerative diseases, autoimmune renal disease, dense deposit disease (ODD), and systemic autoimmune diseases.

In certain embodiments, nonlimiting examples of FHR3 mediated diseases or disorders include nephropathy, age-related macular degeneration, atypical hemolytic uremic syndrome (aHUS), autoimmune form of hemolytic uremic syndrome, hepatocellular carcinoma (HCC), C₃ glomerulopathy, paroxysmal nocturnal hemoglobinuria, Polymyalgia rheumatica, rheumatoid arthritis, meningococcal sepsis, and SLE (Systemic lupus erythematosus).

In certain embodiments, the present invention provides compounds that utilize receptor mediated endocytosis to eliminate or decrease level of complement factor H-related protein 3 (FHR3) from the plasma.

In certain embodiments, the FHR3 Targeting Ligand is selected from:

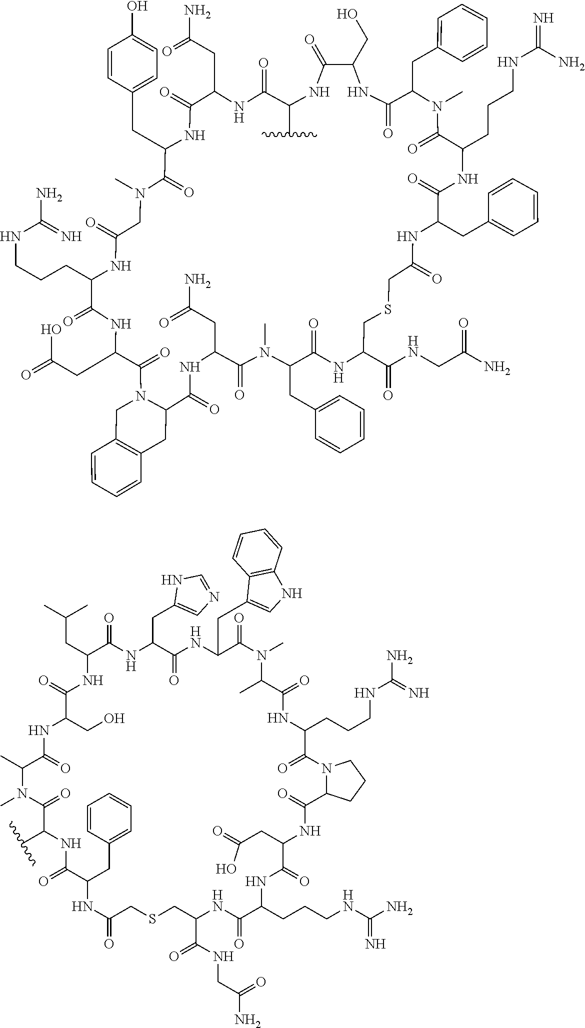 [SMILES: *C1NC(=O)C(CO)NC(=O)C(Cc2ccccc2)N(C)C(=O)C(CCCNC(=N)N)NC(=O)C(Cc2ccccc2)NC(=O)CSCC(C(=O)NCC(N)=O)NC(=O)C(Cc2ccccc2)N(C)C(=O)C(CC(N)=O)NC(=O)C2Cc3ccccc3CN2C(=O)C(CC(=O)O)NC(=O)C(CCCNC(=N)N)NC(=O)CN(C)C(=O)C(Cc2ccc(O)cc2)NC(=O)C(CC(N)=O)NC1=O ; *C1NC(=O)C(Cc2ccccc2)NC(=O)CSCC(C(=O)NCC(N)=O)NC(=O)C(CCCNC(=N)N)NC(=O)C(CC(=O)O)NC(=O)C2CCCN2C(=O)C(CCCNC(=N)N)NC(=O)C(C)N(C)C(=O)C(Cc2c[nH]c3ccccc23)NC(=O)C(Cc2cnc[nH]2)NC(=O)C(CC(C)C)NC(=O)C(CO)NC(=O)C(C)N(C)C1=O]

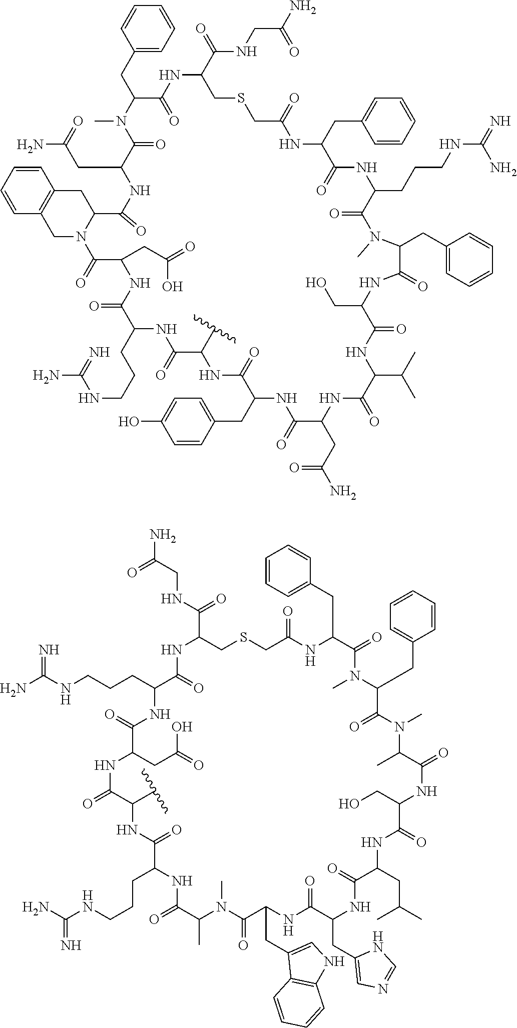 [SMILES: *C1NC(=O)C(Cc2ccc(O)cc2)NC(=O)C(CC(N)=O)NC(=O)C(C(C)C)NC(=O)C(CO)NC(=O)C(Cc2ccccc2)N(C)C(=O)C(CCCNC(=N)N)NC(=O)C(Cc2ccccc2)NC(=O)CSCC(C(=O)NCC(N)=O)NC(=O)C(Cc2ccccc2)N(C)C(=O)C(CC(N)=O)NC(=O)C2Cc3ccccc3CN2C(=O)C(CC(=O)O)NC(=O)C(CCCNC(=N)N)NC1=O ; *C1NC(=O)C(CCCNC(=N)N)NC(=O)C(C)N(C)C(=O)C(Cc2c[nH]c3ccccc23)NC(=O)C(Cc2cnc[nH]2)NC(=O)C(CC(C)C)NC(=O)C(CO)NC(=O)C(C)N(C)C(=O)C(Cc2ccccc2)N(C)C(=O)C(Cc2ccccc2)NC(=O)CSCC(C(=O)NCC(N)=O)NC(=O)C(CCCNC(=N)N)NC(=O)C(CC(=O)O)NC1=O]

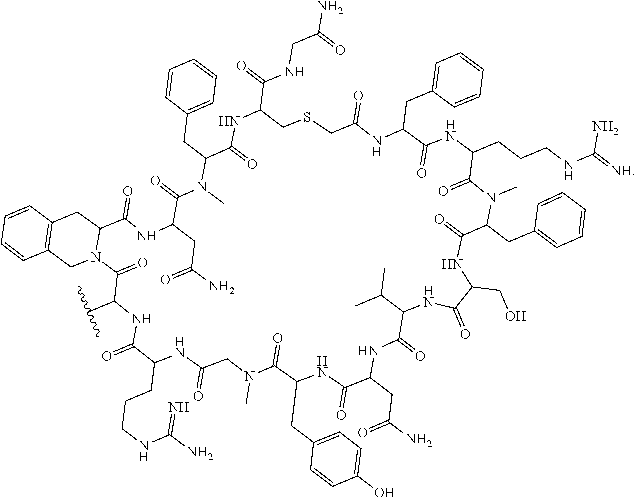 [SMILES: *C1NC(=O)C(CCCNC(=N)N)NC(=O)CN(C)C(=O)C(Cc2ccc(O)cc2)NC(=O)C(CC(N)=O)NC(=O)C(C(C)C)NC(=O)C(CO)NC(=O)C(Cc2ccccc2)N(C)C(=O)C(CCCNC(=N)N)NC(=O)C(Cc2ccccc2)NC(=O)CSCC(C(=O)NCC(N)=O)NC(=O)C(Cc2ccccc2)N(C)C(=O)C(CC(N)=O)NC(=O)C2Cc3ccccc3CN2C1=O]

In certain embodiments, the FHR3 compound is selected from:

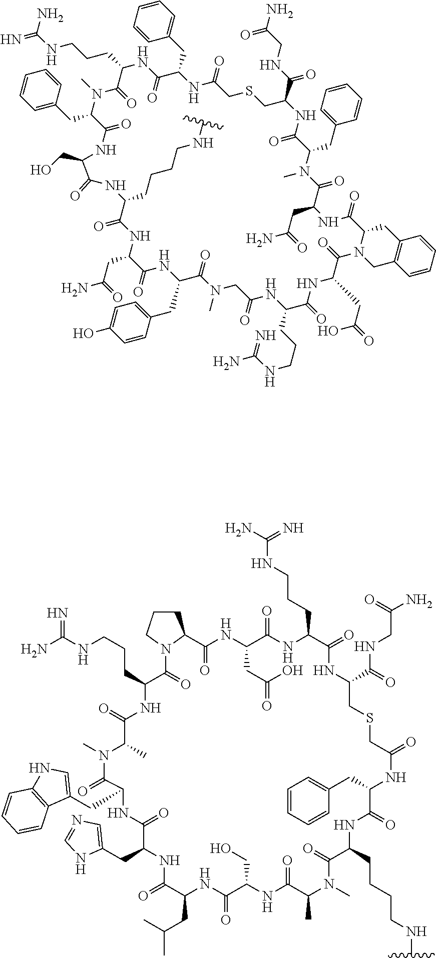

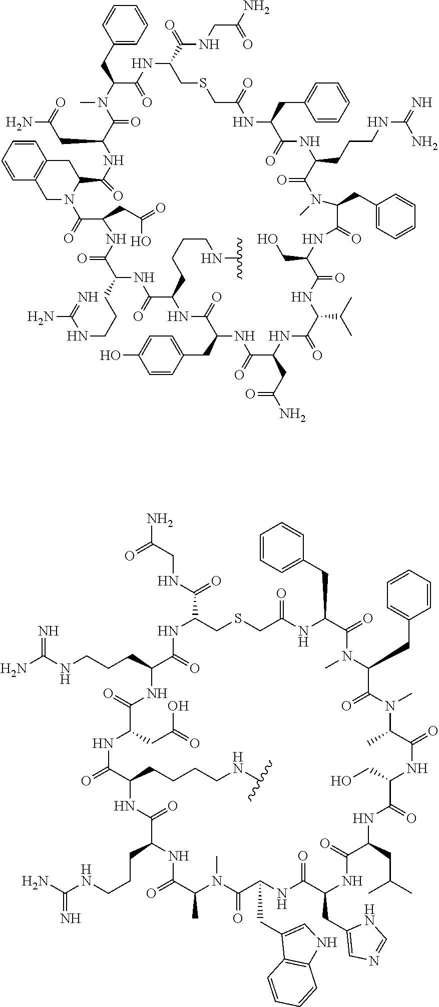

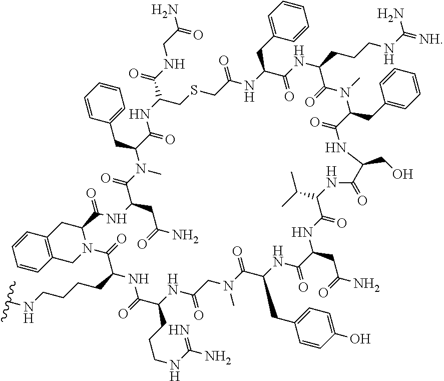

### Tau Protein

In some embodiments, the Target Extracellular Protein is tau protein. The accumulation of tau in the brain causes aggregates that are associated with Alzheimer's and other tauopathies.

Non-limiting examples of Tau Protein targeting ligands include:

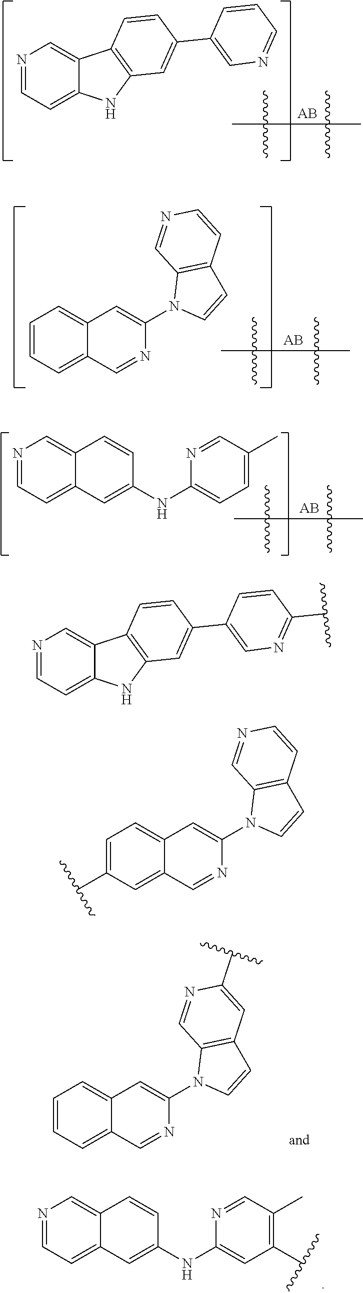

### IL-21

In some embodiments, the Target Extracellular Protein is human interleukin-21 (IL-21) (UniProtKB-Q9HBE4 (IL21_HUMAN)). IL-21 is an immunoregulatory cytokine. IL-21 has been implicated in a number of autoimmune disorders, including Sjogren's syndrome, systemic lupus erythematosus, type 1 diabetes, multiple sclerosis, rheumatoid arthritis, and inflammatory bowel disease.

The Protein Data Bank website provides the crystal structure of IL-21 searchable by 20QP (Bondensgaard, K., et al., J Biol Chem., 2007, 282 23326-23336); and 4NZD (Hamming et al.); as well as the crystal structure of IL-21 bound to various compounds searchable by 3TGX (Hamming, O. J., et al., J Biol Chem., 2012, 287 (12), 9454-9460).

Representative IL-21 Targeting Ligands are described in FIG. 1. Additional IL-21 Targeting Ligands can be found in, for example, U.S. Pat. No. 9,701,663, which is incorporated herein by reference.

### IL-22

In some embodiments, the Target Extracellular Protein is human interleukin-22 (IL-22) (UniProtKB-Q9GZX6 (IL22_HUMAN)). IL-22 is a member of IL-10 family cytokines that is produced by many different types of lymphocytes including both those of the innate and adaptive immune system. IL-22 has been implicated in a number of autoimmune disorders, including, but not limited to, graft versus host disease (GVHD), psoriasis, rheumatoid arthritis, atopic dermatitis, and asthma.

The Protein Data Bank website provides the crystal structure of IL-22 searchable by 1M4R (Nagem, R. A. P., et al., Structure, 2002, 10 1051-1062); as well as the crystal structure of IL-22 bound to various compounds searchable by 3DGC (Jones, B. C. et al., Structure, 2008, 16 1333-1344).

Representative IL-22 Targeting Ligands are described in FIG. 1. Additional IL-22 Targeting Ligands can be found in, for example, U.S. Pat. No. 9,701,663, which is incorporated herein by reference.

### IL-10

In some embodiments, the Target Extracellular Protein is human interleukin-10 (IL-10) (UniProtKB-P22301 (IL10_HUMAN)). IL-10 is an inflammatory cytokine. IL-10 has been implicated in tumor survival and protection against cytotoxic chemotherapeutic drugs.

The Protein Data Bank website provides the crystal structure of IL-10 searchable by 21LK (Zdanov, A et al., Protein Sci., 1996, 5 1955-1962); 11LK (Zdanov, A. et al., Structure, 1995, 3 591-601); 2H24 (Yoon, S. I., et al., J Biol Chem., 2006, 281 35088-35096) and 3LQM (Yoon, S. I., et al., Structure, 2010, 18 638-648). Additionally, Zdanov, A., et al, provides insight into crystal structure of IL-10 (Zdanov A., Current Pharmaceutical design, 2004, 10, 3873-3884).

Representative IL-10 Targeting Ligands are provided in FIG. 1. Additional IL-10 Targeting Ligands can be found, for example, in ACS Chem Biol 11:2105-11 (2016), which is incorporated herein by reference.

### IL-5

In some embodiments, the Target Extracellular Protein is human interleukin-5 (IL-5) (UniProtKB-P05113 (IL5_HUMAN)). IL-5 is a cytokine that regulates eosinophil maturation, recruitment, and survival. IL-5 has been implicated in a number of allergic disorders, including, but not limited to, asthma, nasal polyposis, atopic dermatitis, eosinophilic esophagitis, hypereosinophilic syndrome, and Churg-Strauss syndrome.

The Protein Data Bank website provides the crystal structure of IL-5 searchable by 1HUL (Milburn, M. V., Nature, 1993, 363, 172-176) and 3VA2 (Kusano et al., Protein Sci., 2012, 21 (6), 850-864); as well as the crystal structure of IL-5 bound to various compounds searchable by 1OBX and 1OBZ (Kang, B. S., et al., Structure, 2003, 11, 845).

Representative IL-5 Targeting Ligands are provided in FIG. 1. Additional IL-5 Targeting Ligands can be found, for example, in Bioorg Med Chem 18:4441-5 (2010); Bioorg Med Chem 18:4625-9 (2011); Bioorg Med Chem 21:2543-50 (2013); Eur J Med Chem 59:31-8 (2013); Bioorg Med Chem 23:2498-504 (2015); Bioorg Med Chem 20:5757-62 (2012); each of which is incorporated by reference herein.

### IL8

In some embodiments, the Target Extracellular Protein is human interleukin-8 (IL-8) (UniProtKB-P10145 (IL8_HUMAN)). IL-8 is a chemotactic factor that attracts neutrophils, basophils, and T-cells, but not monocytes. It is also involved in neutrophil activation. It is released from several cell types in response to an inflammatory stimulus. IL-8 has been implicated in the promotion of tumor progression, immune escape, epithelial-mesenchymal transition, and recruitment of myeloid-derived suppressor cells. Studies have demonstrated that high serum IL-8 levels correlate with poor prognosis in many malignant tumors. Preclinical studies have shown that IL-8 blockade may reduce mesenchymal features in tumor cells, making them less resistant to treatment.

The Protein Data Bank website provides the crystal structure of IL-8 searchable by 31L8 (Baldwin, E. T., et al., Proc Natl Acad Sci USA, 1991, 88, 502-506); and 11L8 and 21L8 (Clore, G. M., et al., Biochemistry, 1990, 29, 1689-1696); as well as the crystal structure of IL-8 bound to various compounds searchable by 11LP and 11LQ (Skelton, N, J., et al., Structure, 1999, 7, 157-168); and 1ROD (Sticht, H., et al., Eur J Biochem., 1996, 235, 26-35); 4XDX (Ostrov et al.,) and 5WDZ (Beckamp, S., J Biomol NMR, 2017, 69, 111-121).

Representative IL-8 Targeting Ligands are provided in FIG. 1. Additional IL-8 Targeting Ligands can be found in, for example, Bioorg Med Chem Lett 19:4026-30 (2009), which is incorporated by reference herein.

### Cholinesterase

In some embodiments, the Target Extracellular Protein is human cholinesterase (UniProtKB-P06276 (CHLE_HUMAN)). Cholinesterase contributes to the inactivation of the neurotransmitter acetylcholine. Inhibition of cholinesterase results in increased levels of acetylcholine in the synaptic cleft (the space between two nerve endings). The main use of cholinesterase inhibitors is for the treatment of dementia in patients with Alzheimer's disease. People with Alzheimer's disease have reduced levels of acetylcholine in the brain. Cholinesterase inhibitors have been shown to have an effect on dementia symptoms such as cognition.

The Protein Data Bank website provides the crystal structure of cholinesterase searchable by 1POI and 1POQ (Nicolet, Y., et al., J Biol Chem., 2003, 278, 41141-41147); as well as the crystal structure of cholinesterase bound to various compounds searchable by 1POM and 1POP (Nicolet, Y., et al., J Biol Chem., 2003, 278, 41141-41147); 2J4C (Frasco, M. F., et al., FEBS J., 2007, 274 1849); 4BDT, 4BDS (Nachon, F., et al., Biochem J, 2013, 453, 393-399); 1GQR and 1GQS (Bar-on, P., et al., Biochemistry, 2002, 41, 3555); 3DJY and 3DKK (Carletti, E., et al., J Am Chem Soc., 2008, 130, 16011-16020); 4AXB, 4B00, 4BOP, and 4BBZ (Wandhammer, M., et al., Chem Biol Interact., 2013, 203, 19); 1DX6 (Greenblatt, H. M., et al., FEBS Lett., 1999, 463 321); 1GPK and 1GPN (Dvir, H., et al., Biochemistry, 2002, 41, 10810); 6CQY (Bester, S. M., et al., Chem Res Toxicol., 2018, 31, 1405-1417); 1XLV and 1XLW (Nachon, F., et al., Biochemistry, 2005, 44, 1154-1162); 2Y1K (Carletti, E., et al., Chem Res Toxicol., 2011, 24, 797); and 2WIG, 2WIJ, 2WIK, 2WIL, and 2WSL (Carletti, E., et al., Biochem J., 2009, 421, 97-106). Additionally, Ahmad et al., provides insight into the isolation, crystal structure determination and cholinesterase inhibitory potential of isotalatizidine hydrate from delphinium denudatum (Ahmad H., et al., Journal Pharmaceutical Biology, 2016, 55 (1), 680-686).

Representative cholinesterase Targeting Ligands are provided in FIG. 1. Additional Targeting Ligands can be found in, for example, ACS Med Chem Lett 4:1178-82 (2013); J Med Chem 49:3421-5 (2006); Eur J Med Chem 55:23-31 (2012); J Med Chem 51:3154-70 (2008); J Med Chem 46:1-4 (2002); Eur J Med Chem 126:652-668 (2017); Biochemistry 52:7486-99 (2013); Bioorg Med Chem 23:1321-40 (2015); which are each incorporated herein by reference.

### C—C Motif Chemokine Ligand 2 (CCL2)

Grygiel et al., provides insight into the synthesis by native chemical ligation and crystal structure of human CCL2 (Grygiel, T. L., et al., Biopolymers, 2010, 94 (3), 350-9).

In some embodiments, the Target Extracellular Protein is human C—C motif chemokine ligand 2 (CCL2) (UniProtKB-P13500 (CCL2_HUMAN)). CCL2 acts as a ligand for C—C chemokine receptor CCR2. CCL2 signals through binding and activation of CCR2 and induces a strong chemotactic response and mobilization of intracellular calcium ions. CCL2 exhibits a chemotactic activity for monocytes and basophils but not neutrophils or eosinophils.

CCL2 has been implicated in the recruitment of monocytes into the arterial wall during the disease process of atherosclerosis.

Representative CCL2 Targeting Ligands are provided in FIG. 1. Additional CCL2 Targeting Ligands can be found in, for example, J Med Chem 56:7706-14 (2013), which is incorporated herein by reference.

### Carboxypeptidase B2

In some embodiments, the Target Extracellular Protein is human carboxypeptidase B2 (UniProtKB-Q961Y4 (CBPB2_HUMAN)). Carboxypeptidase B2, also known as thrombin activatable fibrinolysis inhibitor (TAFIa), cleaves C-terminal arginine or lysine residues from biologically active peptides such as kinins or anaphylatoxins in the circulation thereby regulating their activities. It down-regulates fibrinolysis by removing C-terminal lysine residues from fibrin that has already been partially degraded by plasmin. Carboxypeptidase B2 has been implicated and targeted to inhibit thrombosis.

The Protein Data Bank website provides the crystal structure of carboxypeptidase B2 (also known as thrombin-activatable fibrinolysis inhibitor (TAFI)) searchable by 3D₆₆ (Marx, P. F., et al., Blood, 2008, 112, 2803-2809); 3DGV (Anand, K., et al., JBC, 2008, 283, 29416-29423); and 1KWM (Barbosa Pereira, P. J., et al., J Mol Biol., 2002, 321, 537-547); as well as the crystal structure of TAFI bound to various compounds searchable by 3D₆₇ (Marx, P. F., et al., Blood, 2008, 112, 2803-2809); 5HVF, 5HVG, 5HVH (Zhou, X., et al., J Thromb Haemost., 2016, 14, 1629-1638); and 3LMS (Sanglas, L., et al., J Thromb Haemost., 2010, 8, 1056-1065). Additionally, Schreuder et al., provides insight into the interaction of TAFI and anabaenopeptin, a highly potent inhibitor of TAFI (Schreuder, H., et al., Sci Rep., 2016, 6, 32958).

Representative carboxypeptidase B2 Targeting Ligands are provided in FIG. 1. Additional carboxypeptidase B2 Targeting Ligands can be found in, for example, Bioorg Med Chem Lett 20:92-6 (2010), J Med Chem 50:6095-103 (2007), Bioorg Med Chem Lett 14:2141-5 (2004), J Med Chem 58:4839-44 (2015), J Med Chem 55:7696-705 (2012), J Med Chem 59:9567-9573 (2016), Bioorg Med Chem Lett 17:1349-54 (2007), U.S. Pat. Nos. 9,662,310, 8,609,710, 9,688,645, J Med Chem 46:5294-7 (2003), each of which is incorporated herein by reference.

### Neutrophil Elastase

In some embodiments, the Target Extracellular Protein is human neutrophil elastase (UniProtKB-P08246 (ELNE_HUMAN)). Neutrophil elastase modifies the functions of natural killer cells, monocytes and granulocytes. Inhibits C₅a-dependent neutrophil enzyme release and chemotaxis.

Neutrophil elastase has been implicated in a number of disorders, including lung disease, chronic obstructive pulmonary disease, pneumonia, respiratory distress, and acute lung injury (ALI), and cystic fibrosis, as well as chronic kidney disease.

The Protein Data Bank website provides the crystal structure of human neutrophil elastase bound to various compounds searchable by 3Q76 and 3Q77 (Hansen, G., et al., J.Mol.Biol., 2011, 409, 681-691); 5ABW (Von Nussbaum, et al., Bioorg Med Chem Lett., 2015, 25, 4370-4381); 1BOF (Cregge, R. J., et al., J Med Chem., 1998, 41, 2461-2480); 1H₁B (Macdonald, S. J. F., et al., J Med Chem., 2002, 45, 3878); 2Z7F (Koizumi, M., et al., J Synchrotron Radiat., 2008, 15 308-311); 5A09, 5A0A, 5AOB, and 5AOC (Von Nussbaum, F., et al., Chem Med Chem., 2015, 10, 1163-1173); 5A8X, 5A8Y and 5A8Z (Von Nussbaum, F., et al., ChemMedChem., 2016, 11, 199-206); 1HNE (Navia, M. A., et al., Proc Natl Acad Sci USA, 1989, 86, 7-11); 6F5M (Hochscherf, J., et al., Acta Crystallogr F Struct Biol Commun., 2018, 74, 480-489); and 4WVP (Lechtenberg, B. C., et al., ACS Chem Biol., 2015, 10, 945-951).

Representative neutrophil elastase Targeting Ligands are provided in FIG. 1. Additional neutrophil elastase Targeting Ligands can be found in, for example, J Med Chem 53:241-53 (2010), J Med Chem 38:739-44 (1995), J Med Chem 37:2623-6 (1994), J Med Chem 38:4687-92 (1995), J Med Chem 45:3878-90 (2002), Bioorg Med Chem Lett 5:105-109 (1995), Bioorg Med Chem Lett 11:243-6 (2001), J Med Chem 40:1906-18 (1997), Bioorg Med Chem Lett 25:4370-81 (2015), U.S. Pat. Nos. 8,569,314, 9,174,997, 9,290,457, each of which is incorporated herein by reference.

### Factor Xa

In some embodiments, the Target Extracellular Protein is human Factor Xa (UniProtKB-P00742 (FA10_HUMAN)). Factor Xa is a vitamin K-dependent glycoprotein that converts prothrombin to thrombin in the presence of factor Va, calcium and phospholipid during blood clotting.

Factor X has been implicated in the development of deep vein thrombosis and acute pulmonary embolism, and the risk of stroke and embolism in people with nonvalvular atrial fibrillation.

The Protein Data Bank website provides the crystal structure of Factor Xa bound to various compounds searchable by 1G2L and 1G2M (Nar, H., et al., Structure, 2001, 9, 29-38); 2PR³ (Nan huis, C. A., et al., Chem Biol Drug Des., 2007, 69, 444-450); 2UWP (Young, R. J., et al., Bioorg Med Chem Lett., 2007, 17, 2927); 2VVC, 2VVV, 2VVU, 2VWL, 2VWM, 2VWN and 2VWO (Zbinden, K. G., et al., Eur J Med Chem., 2009, 44, 2787); 4Y6D, 4Y71, 4Y7A, 4Y7B, 4zh8, 4ZHA (Convery, M.A. et al.); 4Y76, 4Y79, 2J94 and 2J95 (Chan, C., et al., J Med Chem., 2007, 50 1546-1557); 1FAX (Brandstetter, H., et al., J Biol Chem., 1996, 271, 29988-29992); 2JKH (Salonen, L. M., et al., Angew Chem Int Ed Engl., 2009, 48, 811); 2PHB (Kohrt, J. T., et al., Chem Biol Drug Des., 2007, 70, 100-112); 2W26 (Roehrig, S., et al., J Med Chem., 2005, 48, 5900); 2Y5F, 2Y5G and 2Y5H (Salonen, L. M., et al., Chemistry, 2012, 18, 213); 3Q3K (Yoshikawa, K., et al., Bioorg Med Chem Lett., 2011, 21, 2133-2140); 2BMG (Matter, K., et al., J Med Chem., 2005, 48, 3290); 2BOH, 2BQ6 2BQ7, and 2BQW (Nazare, M., et al., J Med Chem., 2005, 48, 4511); 2CJI (Watson, N. S., et al, Bioorg Med Chem Lett., 2006, 16, 3784); 2J2U, 2J34, 2J38, 2J41 (Senger, S., et al., Bioorg Med Chem Lett., 2006, 16 5731); 31IT (Yoshikawa, K., et al.,Bioorg Med Chem., 2009, 17 8221-8233); 1EZQ, 1FOR and 1FOS (Maignan, S., et al., J Med Chem., 2000, 43, 3226-3232); 1FJS (Adler, M., et al., Biochemistry, 2000, 39, 12534-12542); 1KSN (Guertin, K. R., et al., Bioorg Med Chem Lett., 2002, 12, 1671-1674); INFU, 1NFW, INFX and INFY (Maignan, S., et al., J Med Chem., 2003, 46, 685-690); 2XBV, 2XBW, 2XBX, 2XBY, 2XC0, 2XC4 and 2XC5 (Anselm, L., et al., Bioorg Med Chem Lett., 2010, 20, 5313); 4A7I (Nazare, M., et al., Angew Chem Int Ed Engl., 2012, 51, 905); 4BTI, 4BTT and 4BTU (Meneyrol, L., et al., J Med Chem., 2013, 56, 9441); 3FFG, 3KQB, 3KQC, 3KQD and 3KQE (Quan, M. L., et al., Bioorg Med Chem Lett., 2010, 20, 1373-1377); 2P93, 2P94 and 2P95 (Qiao, J. X., et al., Bioorg Med Chem Lett., 2007, 17, 4419-4427); 1V3X (Haginoya, N., et al., J Med Chem., 2004, 47, 5167-5182); 2P16 (Pinto, D. J. P., et al., J Med Chem., 2007, 50, 5339-5356); 2RAO (Lee, Y. K., et al., J Med Chem., 2008, 51, 282-297); 3SW2 (Shi, Y., et al., Bioorg Med Chem Lett., 2011, 21, 7516-7521); 2VH₆ (Young, R. J., et al., Bioorg Med Chem Lett., 2008, 18, 23); 2WYG and 2WYJ (Kleanthous, S., et al., Bioorg Med Chem Lett., 2010, 20, 618); 2Y7X (Watson, N. S., et al., Bioorg Med Chem Lett., 2011, 21, 1588); 2Y7Z, 2Y80, 2Y81 and 2Y82 (Young, R. J., et al., Bioorg Med Chem Lett., 2011, 21, 1582); 3KL6 (Fujimoto, T., et al., J Med Chem., 2010, 53, 3517-3531); 3LIW (Meuller, M. M., et al., Biol. Chem., 2003, 383, 1185); 5KOH (Schweinitz, A., et al., Med Chem., 2006, 2, 349-361); 1XKA and 1XKB (Kamata, K., et al., Proc Natl Acad Sci USA, 1998, 95, 6630-6635); 2E16 and 2E17 (Nagata, T., et al., Bioorg Med Chem Lett., 2007, 17, 4683-4688); 2P3T (Ye, B., et al., J Med Chem., 2007, 50, 2967-2980); 1MQ5 and 1MQ6 (Adler, M., et al., Biochemistry, 2002, 41, 15514-15523); 3K9X and 3HPT (Shi, Y., et al., Bioorg Med Chem Lett., 2009, 19, 6882-6889); 3CEN (Corte, J. R., et al., Bioorg Med Chem Lett., 2008, 18, 2845-2849); 2W31 and 2W3K (Van Huis, C.A., et al., Bioorg Med Chem., 2009, 17, 2501); 2H₉E (Murakami, M. T., et al., J Mol Biol., 2007, 366, 602-610); 1WU1 and 2DIJ (Komoriya, S., et al., Bioorg Med Chem., 2005, 13, 3927-3954); 2G00 (Pinto, D. J. P., et al., Bioorg Med Chem Lett., 2006, 16, 5584-5589); 3M36 and 3M37 (Pruitt, J. R. et al., J Med Chem., 2003, 46, 5298-5315); 3CS7 (Qiao, J. X., et al., Bioorg Med Chem Lett., 2008, 18, 4118-4123); 1Z6E (Quan, M. L., et al., J Med Chem., 2005, 48, 1729-1744); 2FZZ (Pinto, D. J. P., et al., Bioorg Med Chem Lett., 2006, 16, 4141-4147); and 3ENS (Shi, Y., et al., J Med Chem., 2008, 51, 7541-7551).

Representative Factor Xa Targeting Ligands are provided in FIG. 1. Additional Factor Xa Targeting Ligands can be found in, for example, Bioorg Med Chem Lett 20:5313-9 (2010), Bioorg Med Chem Lett 13:679-83 (2003), J Med Chem 44:566-78 (2001), J Med Chem 50:2967-80 (2007), J Med Chem 38:1511-22 (1995), Bioorg Med Chem Lett 18:2845-9 (2008), J Med Chem 53:6243-74 (2010), Bioorg Med Chem Lett 18:2845-9 (2008), Bioorg Med Chem 16:1562-95 (2008), each of which is incorporated herein by reference.

### Factor XI

In some embodiments, the Target Extracellular Protein is human Factor XI UniProtKB-P03951 (FA11_HUMAN). Factor XI triggers the middle phase of the intrinsic pathway of blood coagulation by activating factor IX.

Factor XI has been implicated in the development of deep vein thrombosis and acute pulmonary embolism, and the risk of stroke and embolism in people with nonvalvular atrial fibrillation.

The Protein Data Bank website provides the crystal structure of Factor XI bound to various compounds searchable by 1ZSL, IZTJ, IZTK, and IZTL (Nagafuji, P., et al.,); 1ZOM (Lin, J., et al., J Med Chem., 2006, 49, 7781-7791); 5EOK and 5EOD (Wong, S. S., et al., Blood, 2016, 127, 2915-2923); 1ZHM, 1ZHP and 1ZHR (Jin, L., et al., Acta Crystallogr D Biol Crystallogr., 2005, 61, 1418-1425); 1ZMJ, 1ZLR, 1ZML and 1ZMN (Lazarova, T.I., Bioorg Med Chem Lett., 2006, 16, 5022-5027); 1ZRK, 1ZSJ and 1ZSK (Guo, Z., et al); 4CRA, 4CRB, 4CRC, 4CRD, 4CRE, 4CRF and 4CRG (Fjellstrom, O., et al., PLOS One, 2015, 10, 13705); 3SOR and 3SOS (Fradera, X., et al., Acta Crystallogr Sect F Struct Biol Cryst Commun., 2012, 68, 404-408); 1ZPB, 1ZPC, 2FDA (Deng, H., et. al., Bioorg Med Chem Lett., 2006, 16, 3049-3054); 5WB6 (Wang, C., et al., Bioorg Med Chem Lett., 2017, 27, 4056-4060); 4NA7 and 4NA8 (Quan, M. L., et al., J Med Chem., 2014, 57, 955-969); 4WXI (Corte, J. R., et al., Bioorg Med Chem Lett., 2015, 25, 925-930); 5QTV, 5QTW, 5QTX and 5QTY (Fang, T., et al., Bioorg Med Chem Lett., 2020, 126949-126949); 6COS (Hu, Z., et al., Bioorg Med Chem Lett., 28, 987-992); 5QQP and 5QQO (Clark, C. G., et al., Bioorg Med Chem Lett., 2019, 29, 126604-126604); 5Q0D, 5Q0E, 5QOF, 5Q0G, and 5Q0H (Corte, J. R., et al., Bioorg Med Chem Lett., 2017, 27, 3833-3839); 5QCK, 5QCL, 5QCM, and 5QCN (Pinto, D. J. P., et al., J Med Chem., 2017, 60, 9703-9723); 5TKS and 5TKU (Corte, J. R., et al., J Med Chem., 2017, 60, 1060-1075); 1XXD and 1XX9 (Jin, L., et al., J Biol Chem., 2005, 280, 4704-4712); 5QTT and 5QTU (Corte, J. R., et al., J Med Chem., 2019, 63, 784-803); 4TY6, 4TY7 (Hangeland, J. J., et al., J Med Chem., 2014, 57, 9915-9932); 4X6M, 4X6N, 4X60, and 4X6P (Pinto, D. J. P., et al., Bioorg Med Chem Lett., 2015, 25, 1635-1642); and 5EX_{M} (Corte, J. R., et al., Bioorg Med Chem., 2016, 24, 2257-2272). Additionally, A1-Horani et al., provides insight into a review of patent literature regarding Factor Xia inhibitors (A1-Horani et al., Expert Opin Ther Pat. 2016; 26 (3), 323-345).

Representative Factor XI Targeting Ligands are provided in FIG. 1. Additional Factor XI Targeting Ligands can be found in, for example, U.S. Pat. Nos. 9,783,530, 10,143,681, 10,214,512, ACS Med Chem Lett 6:590-5 (2015), J Med Chem 60:9703-9723 (2017), J Med Chem 60:9703-9723 (2017), U.S. Pat. No. 9,453,018 (2016), J Med Chem 60:1060-1075 (2017), J Med Chem 57:955-69 (2014), each of which is incorporated herein by reference.

In certain embodiments the Factor XI Targeting Ligand is selected from:

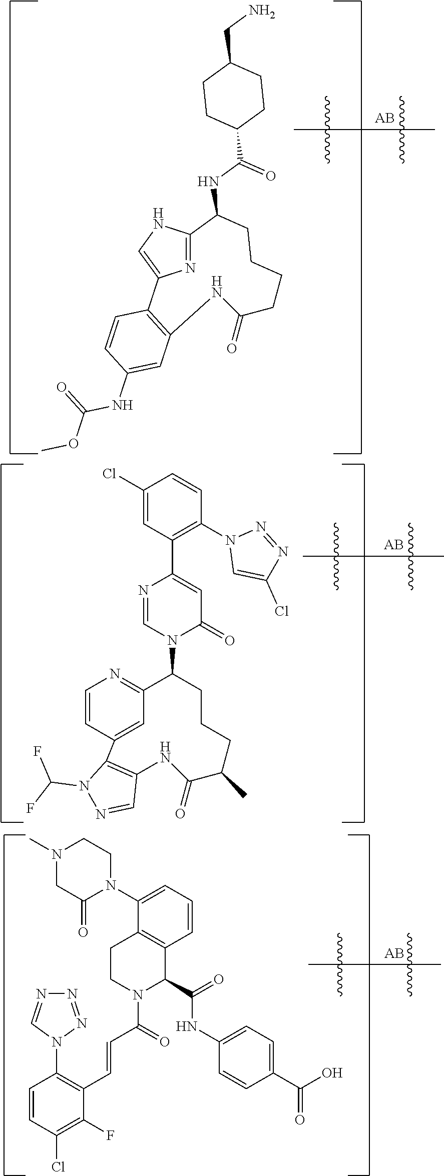

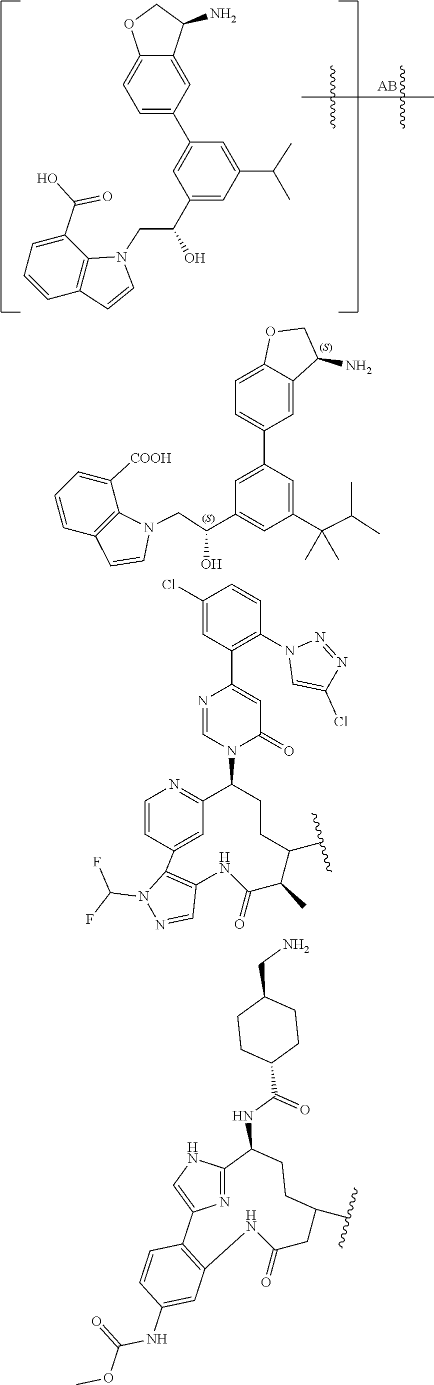

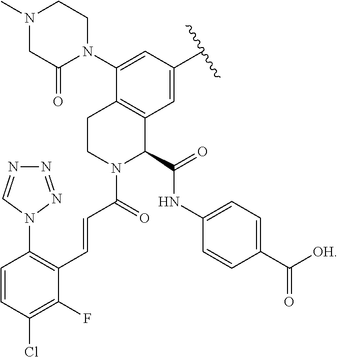 [SMILES: *c1cc2c(c(N3CCN(C)CC3=O)c1)CCN(C(=O)/C=C/c1c(-n3cnnn3)ccc(Cl)c1F)[C@@H]2C(=O)Nc1ccc(C(=O)O)cc1]

In certain embodiments the Factor XI Targeting Ligand is described in J Med Chem 61 (17), 7425-7447 (2018) or J Med Chem (2020) Structure-based design and pre-clinical characterization of selective and orally bioavailable Factor Xia inhibitors: demonstrating the power of an integrated S1 protease family approach.

### Factor XII

In some embodiments, the Target Extracellular Protein is human Factor XII (UniProtKB-P00748 (FA12_HUMAN)). Factor XII is a serum glycoprotein that participates in the initiation of blood coagulation, fibrinolysis, and the generation of bradykinin and angiotensin. Prekallikrein is cleaved by factor XII to form kallikrein, which then cleaves factor XII first to alpha-factor XIIa and then trypsin cleaves it to beta-factor XIIa. Alpha-factor XIIa activates factor XI to factor XIa.

Factor XII has been implicated in the development of deep vein thrombosis and acute pulmonary embolism, and the risk of stroke and embolism in people with nonvalvular atrial fibrillation.

The Protein Data Bank website provides the crystal structure of factor XII bound to various compounds searchable by 4XDE and 4XE4 (Pathak, M., et al., J Thromb Haemost., 2015, 13 (4), 580-591); 6GT6 and 6QF7 (Pathak, M., et al., Acta Crystallogr D Struct Biol., 2019, 75, 578-591); and 6B74 and 6B77 (Dementiev, A. A., et al., Blood Adv., 2018, 2, 549-558). Additionally, Pathak et al., provides insight into the crystal structure of factor XII (Pathak, M., et al., J Thromb Haemost., 2015, 13 (4), 580-591).

Representative Factor XII Targeting Ligands are provided in FIG. 1. Additional Factor XII Targeting Ligands can be found in, for example, J Med Chem 60:1151-1158 (2017), J Med Chem 48:2906-15 (2005), J Med Chem 50:5727-34 (2007), J Med Chem 50:1876-85 (2007), Chembiochem 18:387-395 (2017), each of which is incorporated herein by reference.

### Factor XIII

In some embodiments, the Target Extracellular Protein is human Factor XIII UniProtKB-P00488 (F13A_HUMAN)). Factor XIII is activated by thrombin and calcium ion to a transglutaminase that catalyzes the formation of gamma-glutamyl-epsilon-lysine cross-links between fibrin chains, thus stabilizing the fibrin clot. Also cross-link alpha-2-plasmin inhibitor, or fibronectin, to the alpha chains of fibrin.

Factor XIII has been implicated in the development of deep vein thrombosis and acute pulmonary embolism, and the risk of stroke and embolism in people with nonvalvular atrial fibrillation.

The Protein Data Bank website provides the crystal structure of factor XIII searchable by 1FIE (Yee, V. C., et al., Thromb Res., 1995, 78, 389-397); and 1F13 (Weiss, M.S., et al., FEBS Lett., 1998, 423, 291-296); as well as the crystal structure of factor XIII bound to various compounds searchable by 1DE7 (Sadasivan, C., et al., J Biol Chem., 2000, 275, 36942-36948); and 5MHL, 5MHM, 5MHN, and 5MHO (Stieler, M., et al.,). Additionally, Gupta et al., provides insight into the mechanism of coagulation factor XIII activation and regulation from a structure/functional perspective (Gupta, S., et al., Sci Rep., 2016; 6, 30105); and Komaromi et al., provides insight into the novel structural and functional aspect of factor XIII (Komaromi, Z., et al.,. J Thromb Haemost 2011, 9, 9-20).

Representative Factor XIII Targeting Ligands are provided in FIG. 1. Additional Factor XIII Targeting Ligands can be found in, for example, Eur J Med Chem 98:49-53 (2015), J Med Chem 55:1021-46 (2012), J Med Chem 48:2266-9 (2005), each of which is incorporated herein by reference.

### Prothrombin

In some embodiments, the Target Extracellular Protein is human Prothrombin (UniProtKB-P00734 (THRB_HUMAN)). Thrombin, which cleaves bonds after Arg and Lys, converts fibrinogen to fibrin and activates factors V, VII, VIII, XIII, and, in complex with thrombomodulin, protein C. Functions in blood homeostasis, inflammation and wound healing.

Thrombin is involved in blood clot formation and arterial and venous thrombosis, and thromboembolism associated with atrial fibrillation.

The Protein Data Bank website provides the crystal structure of prothrombin searchable by 3NXP (Chen, Z. et al., Proc Natl Acad Sci USA, 2010, 107, 19278-19283); as well as the crystal structure of prothrombin bound to various compounds searchable by 2HPP and 2HPQ (Arni, R. K., et al., Biochemistry, 1993, 32, 4727-4737); 6BJR, 6C₂W (Chinnaraj, M., et al., Sci Rep., 2018, 8, 2945-2945); 5EDK, 5EDM (Pozzi, N., et al., J Biol Chem., 2016, 291, 6071-6082); 3K65 (Adams, T.E., et al., Biochimie, 2016, 122, 235-242); and 6BJR and 6C₂W (Chinnaraj, M. et al., Sci Rep., 2018, 8, 2945-2945). Additionally, Pozzi et al., provides insight into the mechanism and conformational flexibility for the crystal structure of prothrombin (Pozzi, N. et al., J Biol Chem., 2013, 288 (31), 22734-22744); and Zhiwei et al., provides insight into the crystal structure of prothrombin-1 (Zhiwei, C. et al., PNAS, 2010, 107 (45), 19278-19283).

Prothrombin is converted to thrombin, as such the Protein Data Bank website provides the crystal structure of thrombin bound to compounds searchable by 1XMN (Carter, W. J. et al., J.Biol. Chem., 2005, 280, 2745-2749); 4CH₂ and 4CH₈ (Lechtenberg, B. C. et al., J Mol Biol., 2014, 426, 881); 3PO1 (Karle, M. et al., Bioorg Med Chem Lett., 2012, 22, 4839-4843); 3DA9 (Nilsson, M. et al., J Med Chem., 2009, 52, 2708-2715); 2H₉T and 3BF6 (Lima, L.M.T.R. et al., Biochim Biophys Acta., 2009, 1794, 873-881); 3BEF and 3BEI (Gandhi, P. S. et al., Proc Natl Acad Sci USA, 2008, 105, 1832-1837); 3BV9 (Nieman, M.T. et al., J Thromb Haemost., 2008, 6, 837-845); 2HWL (Pineda, A. O. et al., Biophys Chem., 2007, 125, 556-559); 2AFQ (Johnson, D. J. D. et al., Biochem J., 2005, 392, 21-28); 1SHH (Pineda, A. O. et al., J Biol Chem., 2004, 279, 31842-31853); 1JWT (Levesque, S. et al., Bioorg Med Chem Lett., 2001, 11, 3161-3164); 1G37 (Bachand, B. et al., Bioorg Med Chem Lett., 2001, 11, 287-290); 1EOJ and 1EOL (Slon-Usakiewicz, J. J. et al., Biochemistry, 2000, 39, 2384-2391); 1AWH (Weir, M. P. et al., Biochemistry, 1998, 37, 6645-6657); 1DIT (Krishnan, R. et al., Protein Sci., 1996, 5, 422-433); 1HAO and 1HAP (Padmanabhan, K. et al., Acta Crystallogr D Biol Crystallogr., 1996, 52, 272-282); and 1HBT (Rehse, P. H. et al., Biochemistry, 1995, 34, 11537-11544).

Representative prothrombin Targeting Ligands are provided in FIG. 1. Additional prothrombin Targeting Ligands can be found in, for example, J Med Chem 46:3612-22 (2003), Bioorg Med Chem Lett 12:1017-22 (2002), J Med Chem 40:830-2 (1997), Bioorg Med Chem Lett 15:2771-5 (2005), J Med Chem 42:3109-15 (1999), J Med Chem 47:2995-3008 (2004), Bioorg Med Chem 16:1562-95 (2008), J Med Chem 42:3109-15 (1999), each of which is incorporated herein by reference.

### Coagulation Factor VII

In some embodiments, the Target Extracellular Protein is human coagulation Factor VII (UniProtKB-P08709 (FA7_HUMAN)). Factor VII initiates the extrinsic pathway of blood coagulation. It is a serine protease that circulates in the blood in a zymogen form. Factor VII is converted to Factor VIIa by Factor Xa, Factor XIIa, Factor IXa, or thrombin by minor proteolysis. In the presence of tissue factor and calcium ions, Factor VIIa then converts Factor X to Factor Xa by limited proteolysis. Factor VIIa will also convert Factor IX to Factor IXa in the presence of tissue factor and calcium.

Factor VII is involved in blood clot formation and arterial and venous thrombosis, and thromboembolism associated with atrial fibrillation.

The Protein Data Bank website provides the crystal structure of factor VII bound to various compounds searchable by 2F9B (Rai, R., et al., Bioorg Med Chem Lett., 2006, 16, 2270-2273); 5U6J (Wurtz, N. R., et al., Bioorg Med Chem Lett., 2017, 27, 2650-2654); 5L2Y, 5L2Z, and 5L30 (Ladziata,.U., et al., Bioorg Med Chem Lett., 2016, 26, 5051-5057); 5146 (Glunz, P. W., et al., J Med Chem., 2016, 59, 4007-4018); 4YLQ, 4Z6A, and 4ZMA (Sorensen, A. B., et al., J Biol Chem., 2016, 291, 4671-4683); 4YT6 and 4YT7 (Glunz, P. W., et al., Bioorg Med Chem Lett, 2015, 25, 2169-2173); 4NA9 (Quan, M. L., et al., J Med Chem., 2014, 57, 955-969); 4NG9 (hang, X., et al., ACS Med Chem Lett., 2014, 5, 188-192); 4JZD, 4JZE and 4JZF (Bolton, S. A., et al., Bioorg Med Chem Lett., 2013, 23, 5239-5243); 4JYU and 4JYV (Glunz, P. W., et al., Bioorg Med Chem Lett., 2013, 23, 5244-5248); 41SH (Priestley, E. S., et al., Bioorg Med Chem Lett., 2013, 23, 2432-2435); 41SI (Zhang, X., et al., Bioorg Med Chem Lett., 2013, 23, 1604-1607); 2ZZU (Shiraishi, T., et al., Chem Pharm Bull (Tokyo), 2010, 58, 38-44); 1WV7 and 1WUN (Kadono, S., et al., Biochem Biophys Res Commun., 2005, 327, 589-596); 2ZWL, 2ZPO, (Kadono, S., et al.); 2EC9 (Krishan, R., et al., Acta Crystallogr D Biol Crystallogr., 2007, 63, 689-697); 2PUQ (Larsen, K. S., et al., Biochem J., 2007, 405, 429-438); 2FLR (Riggs, J. R., et al., Bioorg Med Chem Lett., 2006, 16, 3197-3200); 2C₄F (Kohrt, J. T., et al., Bioorg Med Chem Lett., 2006, 16, 1060); 2AEI (Kohrt, J. T. et al., Bioorg Med Chem Lett., 2005, 15, 4752-4756); 1WTG (Kadono, S., et al., Biochem Biophys Res Commun., 2005, 326, 859-865); 1WSS (Kadono, S., et al., Acta Crystallogr Sect F Struct Biol Cryst Commun., 2005, 61, 169-173); 1W7X and 1W8B (Zbinden, K. G., et al., Bioorg Med Chem Lett., 2005, 15, 5344); 1WQV (Kadono, S., et al., Biochem Biophys Res Commun., 2004, 324, 1227-1233); 1Z6J (Schweitzer, B. A., et al., Bioorg Med Chem Lett., 2005, 15, 3006-3011); 1YGC (Olivero, A. G., et al., J Biol Chem., 2005, 280, 9160-9169); 6R²W (Sorensen, A. B., et al., J Biol Chem., 2019, 295, 517-528); 5PA8, 5PA9, 5PAA, 5PAB, 5PAC, 5PAE, 5PAF, 5PAG, 5PAI, 5PAJ, 5PAK, 5PAM, 5PAN, 5PAO, 5PAQ, 5PAR, 5PAS, 5PAT, 5PAU, 5PABV, 5PAW, 5PAX, 5PAY, 5PB0, 5PB1, 5PB2, 5PB3, 5PB4, 5PB5, and 5PB6 (Mayweg, A. V., et al.,); and 5LOS (Li, Z., et al., Nat Commun., 2017, 8, 185-185). Additionally, Kemball-Cook, et al., provides insight into the crystal structure of active site-inhibited factor VIIa (Kemball-Cook, G., et al., J Struct Biol., 1999, 127 (3), 213-23).

Representative Factor VII Targeting Ligands are provided in FIG. 1. Additional Factor VII Targeting Ligands can be found in, for example, U.S. Pat. No. 9,174,974, Bioorg Med Chem Lett 26:5051-5057 (2016), Bioorg Med Chem Lett 11:2253-6 (2001), Bioorg Med Chem Lett 15:3006-11 (2005), Bioorg Med Chem Lett 12:2883-6 (2002), each of which is incorporated herein by reference.

### Coagulation Factor IX

In some embodiments, the Target Extracellular Protein is human coagulation Factor IX (UniProtKB-P00740 (FA9_HUMAN)). Factor IX Factor IX is a vitamin K-dependent plasma protein that participates in the intrinsic pathway of blood coagulation by converting factor X to its active form in the presence of Ca2+ ions, phospholipids, and factor VIIIa.

Factor IX is involved in blood clot formation and arterial and venous thrombosis, and thromboembolism associated with atrial fibrillation.

The Protein Data Bank website provides the crystal structure of factor IX bound to various compounds searchable by 6MV4 (Vadivel, K., et al., J Thromb Haemost., 2019, 17, 574-584); 4ZAE (Zhang, T., et al., Bioorg Med Chem Lett., 2015, 25, 4945-4949); 4YZU and 4ZOK (Parker, D. L., et al., Bioorg Med Chem Lett., 2015, 25, 2321-2325); 5TNO and 5TNT (Sakurada, I., et al., Bioorg Med Chem Lett., 2017, 27, 2622-2628); 5JB8, 5JB9, 5JBA, 5JBB and 5JBC (Kristensen, L. H., et al., Biochem J., 2016, 473, 2395-2411); 3LC3 (Wang, S., et al., J Med Chem., 2010, 53, 1465-1472); 3LC5 (Wang, S., et al., J Med Chem., 2010, 53, 1473-1482); 3KCG (Johnson, D. J. D., et al., Proc Natl Acad Sci USA, 2010, 107, 645-650); INLO (Huang, M., et al., J Biol Chem., 2004, 279, 14338-14346); 1RFN (Hopfner, K. P., et al., Structure, 1999, 7, 989-996); and 6RFK (Sendall, T. J., et al.,).

Representative Factor IX Targeting Ligands are provided in FIG. 1. Additional Factor IX Targeting Ligands can be found in, for example, U.S. Pat. No. 9,409,908, Bioorg Med Chem Lett 25:5437-43 (2015), U.S. Pat. No. 10,189,819, each of which is incorporated herein by reference.

### Fibroblast Growth Factor 1 (FGF1)

In some embodiments, the Target Extracellular Protein is human fibroblast growth factor 1 (FGF1) (UniProtKB-P05230 (FGF1_HUMAN)). FGF1 plays an important role in the regulation of cell survival, cell division, angiogenesis, cell differentiation and cell migration. FGF1 acts as a ligand for FGFR1 and integrins, and binds to FGFR1 in the presence of heparin leading to FGFR1 dimerization and activation via sequential autophosphorylation on tyrosine residues which act as docking sites for interacting proteins, leading to the activation of several signaling cascades. FGF1 induces the phosphorylation and activation of FGFR1, FRS2, MAPK3/ERK1, MAPK1/ERK2 and AKTI. FGF1 can induce angiogenesis. FGF1 has been implicated in oncogenesis, cancer cell proliferation, resistance to anticancer therapies, and neoangiogenesis.

The Protein Data Bank website provides the crystal structure of FGF1 searchable by 2AFG (Blaber, M., et al., Biochemistry, 1996, 35, 2086-2094); and 1BAR (Zhu, X. et al., Science, 1991, 251, 90-93); as well as the crystal structure of FGF1 bound to various compounds searchable by 1AFC (Zhu, X., et al., Structure, 1993, 1, 27-34); 1AX_{M} and 2AX_{M} (DiGabriele, A. D., et al., Nature, 1998, 393, 812-817); 1EVT (Plotnikov, A. N., et al., Cell, 2000, 101, 413-424); 1E00 (Pellegrini, L., et al., Nature, 2000, 407, 1029); and 2ERM (Canales, A., et al., FEBS J, 2006, 273, 4716-4727).

Representative FGF1 Targeting Ligands are provided in FIG. 1. Additional FGF1 Targeting Ligands can be found in, for example, Bioorg Med Chem Lett 18:344-9 (2008), Chembiochem 6:1882-90 (2005), J Med Chem 55:3804-13 (2012), J Med Chem 47:1683-93 (2004), J Med Chem 53:1686-99 (2010,) each of which is incorporated herein by reference.

### Fibroblast Growth Factor 2 (FGF2)

In some embodiments, the Target Extracellular Protein is human fibroblast growth factor 2 (FGF2) (UniProtKB-P09038 (FGF2_HUMAN)). FGF2 acts as a ligand for FGFR1, FGFR2, FGFR3 and FGFR4. FGF2 also acts as an integrin ligand which is required for FGF2 signaling, and plays an important role in the regulation of cell survival, cell division, cell differentiation and cell migration. FGF2 also induces angiogenesis. FGF2 has been implicated in oncogenesis, cancer cell proliferation, resistance to anticancer therapies, and neoangiogenesis.

The Protein Data Bank website provides the crystal structure of FGF2 bound to various compounds searchable by 40EE, 40E F, and 40EG (Li, Y.C., et al., ACS Chem Biol., 2014, 9, 1712-1717); 1EV2 (Plotnikov, A. N., et al., Cell, 2000, 101, 413-424); and 5X10 (Tsao, Y.H.).

Representative FGF2 Targeting Ligands are provided in FIG. 1. Additional FGF2 Targeting Ligands can be found in, for example, U.S. Pat. No. 8,933,099, Bioorg Med Chem Lett 12:3287-90 (2002), Chem Biol Drug Des 86:1323-9 (2015), Bioorg Med Chem Lett 25:1552-5 (2015), each of which is incorporated herein by reference.

### Fibronectin-1

In some embodiments, the Target Extracellular Protein is human fibronectin 1 (FN1) (UniProtKB-P02751 (FINC_HUMAN)). Fibronectin (FN) polymerization is necessary for collagen matrix deposition and is a key contributor to increased abundance of cardiac myofibroblasts (MFs) after cardiac injury. Interfering with FN polymerization may attenuate MF and fibrosis and improve cardiac function after ischemia/reperfusion (I/R) injury.

The Protein Data Bank website provides the crystal structure of fibronectin-1 bound to various compounds searchable by 3M7P (Graille, M., et al., Structure, 2010, 18, 710-718); 3MQL (Erat, M. C., et al., J Biol Chem., 2010, 285, 33764-33770); and 3EJH (Erat, M. C., et al., Proc Natl Acad Sci USA, 2009, 106, 4195-4200).

Representative FN Targeting Ligands are provided in FIG. 1. Additional FN Targeting Ligands can be found in, for example, Bioorg Med Chem Lett 18:2499-504 (2008), which is incorporated herein by reference.

### Kallikrein-1 (KLK1)

In some embodiments, the Target Extracellular Protein is human kallikrein-1 (UniProtKB-P06870 (KLK1_HUMAN)). Glandular kallikreins cleave Met-Lys and Arg-Ser bonds in kininogen to release Lys-bradykinin. Kallikrein has been implicated in adverse reactions in hereditary angioedema (HAE).

The Protein Data Bank website provides the crystal structure of KLK 1 searchable by 1SPJ (Laxmikanthan, G., et al., Proteins, 2005, 58, 802-814); as well as the crystal structure of KLK1 bound to various compounds searchable by 5F8Z, 5F8T, 5F8X, (Xu, M., et al.,); and 6A80 (Xu, M., et al., FEBS Lett., 2018, 592, 2658-2667). Additionally, Katz et al., provides insight into the crystal structure of kallikrein (Katz, B. A., et al., Protein Sci., 1998, 7 (4), 875-85).

Representative kallikrein Targeting Ligands are provided in FIG. 1. Additional kallikrein Targeting Ligands can be found in, for example, U.S. Pat. No. 9,783,530, J Med Chem 38:2521-3 (1995), U.S. Pat. Nos. 9,234,000, 10,221,161, 9,687,479, 9,670,157, 9,834,513, J Med Chem 38:1511-22 (1995), U.S. Pat. No. 10,214,512, each of which is incorporated herein by reference.

### Plasma Kallikrein

In some embodiments, the Target Extracellular Protein is human plasma kallikrein (UniProtKB-P03952 (KLKB1_HUMAN)). Plasma kallikrein cleaves Lys-Arg and Arg-Ser bonds. It activates, in a reciprocal reaction, factor XII after its binding to a negatively charged surface. It also releases bradykinin from HMW kininogen and may also play a role in the renin-angiotensin system by converting prorenin into renin. Plasma kallikrein has been implicated in retinal dysfunction, the development of diabetic macular edema and hereditary angioedema (HAE).

The Protein Data Bank website provides the crystal structure of plasma kallikrein bound to various compounds searchable by 5TJX (Li, Z., et al., ACS Med Chem Lett., 2017, 8, 185-190); 601G and 601S (Patridge, J. R., et al., J Struct Biol., 2019, 206, 170-182); 40GX and 40GY (Kenniston, J. A., et al., J Biol Chem., 2014, 289, 23596-23608); and 5F8T, 5F8X, and 5F8Z (Xu, M., et al.,).

Representative plasma kallikrein Targeting Ligands are provided in FIG. 1. Additional plasma kallikrein Targeting Ligands can be found in, for example, J Med Chem 61:2823-2836 (2018), J Med Chem 55:1171-80 (2012), U.S. Pat. Nos. 8,598,206, 9,738,655, Bioorg Med Chem Lett 16:2034-6 (2006), U.S. Pat. Nos. 9,409,908, 10,144,746, 9,290,485, each of which is incorporated herein by reference.

### Lipoprotein Lipase

In some embodiments, the Target Extracellular Protein is human lipoprotein lipase (UniProtKB-P06858 (LIPL_HUMAN)). Lipoprotein lipase is a key enzyme in triglyceride metabolism. It catalyzes the hydrolysis of triglycerides from circulating chylomicrons and very low density lipoproteins (VLDL), and thereby plays an important role in lipid clearance from the blood stream, lipid utilization and storage. Lipoprotein lipase mediates margination of triglyceride-rich lipoprotein particles in capillaries. Lipoprotein lipase has been implicated in the development of cardiovascular disease and obesity.

The Protein Data Bank website provides the crystal structure of lipoprotein lipase bound to various compounds searchable by 6E7K (Birrane, G., et al., Proc Natl Acad Sci USA, 2018 116 1723-1732).

Representative lipoprotein lipase Targeting Ligands are provided in FIG. 1. Additional lipoprotein lipase Targeting Ligands can be found in, for example, J Med Chem 47:400-10 (2004), which is incorporated herein by reference.

### Matrix Metallopeptidase 1 (MMP-1)

In some embodiments, the Target Extracellular Protein is human matrix metallopeptidase 1 (MMP-1) (UniProtKB-P03956 (MMP1_HUMAN)). MMP-1 cleaves collagens of types I, II, and III at one site in the helical domain. It also cleaves collagens of types VII and X. MMP-1 has been implicated in cardiovascular disease.

The Protein Data Bank website provides the crystal structure of MMP-1 searchable by 3SHI (Bertini, I., et al., FEBS Lett., 2012, 586, 557-567); as well as the crystal structure of MMP-1 bound to various compounds searchable by 4AUO (Manka, S. W., et al., Proc Natl Acad Sci U SA, 2012, 109, 12461); 3MA2 (Grossman, M., et al., Biochemistry, 2010, 49, 6184-6192); and 2JOT (Iyer, S., et al., J.Biol. Chem., 2007, 282, 364). Additionally, Iyer et al., provides insight into the crystal structure of an active form of MMP-1 (lyer, S., et al., J Mol Biol., 2006, 362 (1), 78-88); and Lovejoy et al., provides insight into the crystal structure of MMP1 and the selectivity of collagenase inhibitors (Lovejoy, B., et al., Nat Struct Mol Biol., 1999, 6, 217-221).

Representative MMP-1 Targeting Ligands are provided in FIG. 1. Additional MMP-1 Targeting Ligands can be found in, for example, Bioorg Med Chem Lett 5:1415-1420 (1995), Bioorg Med Chem Lett 16:2632-6 (2006), Bioorg Med Chem Lett 8:837-42 (1999), Eur J Med Chem 60:89-100 (2013), J Med Chem 54:4350-64 (2011), Bioorg Med Chem Lett 8:3251-6 (1999), J Med Chem 42:4547-62 (1999), J Med Chem 61:2166-2210 (2018), J Med Chem 41:1209-17 (1998), which is incorporated herein by reference.

### Macrophage Migration Inhibitory Factor (MIF)

In some embodiments, the Target Extracellular Protein is human macrophage migration inhibitory factor (MIF) (UniProtKB-P14174 (MIF_HUMAN)). MIF is a pro-inflammatory cytokine involved in the innate immune response to bacterial pathogens. The expression of MIF at sites of inflammation suggests a role as mediator in regulating the function of macrophages in host defense. It counteracts the anti-inflammatory activity of glucocorticoids.

MIF has been implicated in tumor progression; systemic inflammation; atherosclerosis; rheumatoid arthritis; and systemic lupus erythematosus, among others.

The Protein Data Bank website provides the crystal structure of MIF searchable by 1MIF (Sun, H—W. et al., Proc Natl Acad Sci USA, 1996, 93, 5191-5196); as well as the crystal structure of MIF bound to various compounds searchable by 6PEG (Cirillo, P. F. et al.,); 5XEJ (Fukushima, K); 6FVE and 6FVH (Sokolov, A. V., et al., Biochemistry (Mosc), 2018, 83, 701-707); 6CB5, 6CBF, 6CBG, and 6CBH (Trivedi-Parmar, V., et al., ChemMedChem., 2018, 13, 1092-1097); 6B1C, 6B1K, 6B2C, (Dawson, T. K., et al., ACS Med Chem Lett., 2017, 8, 1287-1291); 4Z15, 4Z1T and 4Z1U (Singh, A. K., et al, J Cell Mol Med., 2017, 21, 142-153); 5HVS and 5HVT (Cisneros, J. A., et al., J Am Chem Soc., 2016, 138, 8630-8638); 4PKK (Pantouris, G., et al.,); 5J7P and 5J7Q (Cisneros, J. A., et al., Bioorg Med Chem Lett., 2016, 26, 2764-2767); 5B40 (Kimura, H., et al., Chem Biol., 2010, 17, 1282-1294); 4PLU, 4TRF, 4POH, and 4P01 (Pantouris, G., et al., Chem Biol., 2015, 22, 1197-1205); 4WR⁸ and 4WRB (Dziedzic, P., et al., J Am Chem Soc., 2015, 137 2996-3003); 4K9G (Ioannou, K., et al., Int J Oncol., 2014, 45, 1457-1468); 40SF, 3WNR, 3WNS and 3WNT (Spencer, E. S., et al., Eur J Med Chem., 2015, 93, 501-510); 40YQ (Spencer, E. S. et al.,); 3SMB and 3SMC (Crichlow, G. V. et al., Biochemistry, 2012, 51, 7506-7514); 3U18 (Bai, F., et al., J Biol Chem., 2012, 287, 30653-30663); 4F2K (Tyndall, J. D. A., et al., Acta Crystallogr Sect F Struct Biol Cryst Commun., 2012, 68, 999-1002); 31JG and 31JJ (Cho, Y., et al., Proc Natl Acad Sci USA, 2010, 107, 11313-11318); 3L5P, 3L5R, 3L5S, 3L5T, 3L5U, and 3L5V (McLean, L. R. et al., Bioorg Med Chem Lett., 2010, 20, 1821-1824); 3JSF, 3JSG and 3JTU (McLean, L. R., et al., Bioorg Med Chem Lett., 2009, 19, 6717); 3HOF (Crawley, L., et al.); 3CE4 and 3DJI (Crichlow G.V., et al., Biochemistry, 2009, 48, 132-139); 3B9S (Winner, M. et al., Cancer Res., 2008, 68, 7253-7257); 200H, 200W and 200Z (Crichlow, G. V. et al., J Biol Chem., 2007, 282, 23089-23095); 1GCZ and 1GDO (Orita, M. et al., J Med Chem., 2001, 44, 540-547); and 1CA7, 1CGQ and 1P1G (Lubetsky, J. B. et al., Biochemistry, 1999, 38, 7346-7354). Additionally, Sun et al., provides insight into the crystal structure of MIF (Proc Natl Acad Sci U SA., 1996, 28; 93 (11), 5191-6).

Representative MIF Targeting Ligands are provided in FIG. 1. Additional MIF Targeting Ligands can be found in, for example, ACS Med Chem Lett 8:124-127 (2017), J Med Chem 44:540-7 (2001), J Med Chem 52:416-24 (2009), J Med Chem 50:1993-7 (2007), which is incorporated herein by reference.

### Transforming Growth Factor —B2 (TGF-β2)

In some embodiments, the Target Extracellular Protein is human transforming growth factor-β2 (TGF-β2) (UniProtKB-P61812 (TGFB2_HUMAN)). TGF-β2 is a multifunctional protein that regulates various processes such as angiogenesis and heart development. Once activated following release of LAP, TGF-beta-2 acts by binding to TGF-beta receptors (TGFBR1 and TGFBR2), which transduce signal. TGF-β2 expression in the tumor microenvironment has been associated with a poor prognosis, and is implicated in TGF-β2 mediated tumor suppression via T-cell exclusion. TGF-β2 expression has also been implicated in hematological malignancies and fibrosis.

The Protein Data Bank website provides the crystal structure of TGF-β2 searchable by 619J (Del Amo-Maestro L. et al., Sci Rep. 2019, 9, 8660-8660); as well as the crystal structure of TGF-β2 bound to various compounds searchable by 1M9Z (Boesen, C. C., et al. Structure, 2002, 10, 913-919); 5QIN (Zhang, Y. et al., ACS Med Chem Lett., 2018, 9, 1117-1122); 5E8V, 5E8Y, 5E91 and 5E92 (Tebben, A. J. et al., Acta Crystallogr D Struct Biol., 2016, 72, 658-674); 4P7U (Wangkanont, K. et al., Protein Expr Purif., 2015, 115, 19-25); 4XJJ (Wangkanont et al.); and 1KTZ (Hart, P. J., et al., Nat Struct Biol., 2002, 9, 203-208).

Representative TGF-β2 Targeting Ligands are provided in FIG. 1.

### Thrombospondin-1 (TSP-1)

In some embodiments, the Target Extracellular Protein is human thrombospondin-1 (TSP-1) (UniProtKB-P61812 (TGFB2_HUMAN)). TSP1 acts as an angiogenesis inhibitor by stimulating endothelial cell apoptosis, inhibiting endothelial cell migration and proliferation, and regulating vascular endothelial growth factor bioavailability and activity. TSP1 affects tumor immune response, tumor cell behaviors including adhesion, invasion, migration, apoptosis, and proliferation.

TSP-1 expression has been implicated in a number of diseases, including in promoting certain cancers such as breast cancer, prostate cancer, melanoma, SCLC, osteosarcoma, cutaneous squamous cell carcinoma, oral squamous cell carcinoma, papillary thyroid carcinoma, thyroid cancer, medulloblastoma, and fibrotic disorders such as diabetes, liver fibrosis, and in multiple myeloma.

The Protein Data Bank website provides the crystal structure of TSP-1 searchable by 1LSL

(Tan, K. et al., J Cell Biol., 2002, 159, 373-382); 2ES3 (Tan, K., et al., J Biol Chem., 2008, 283, 3932-3941); 1Z78 and 2ERF (Tan, K., et al., Structure, 2006, 14, 33-42); and 3R⁶B (Klenotic, P. A., et al., Protein Expr Purif., 2011, 80, 253-259); as well as the crystal structure of TSP-1 bound to various compounds searchable by 20UH and 2OUJ (Tan, K., et al., J Biol Chem., 2008, 283, 3932-3941); and 1ZA4 (Tan, K., et al., Structure, 2006, 14, 33-42).

Representative TSP-1 Targeting Ligands are provided in FIG. 1.

### CD40 Ligand (CD40L)

In some embodiments, the Target Extracellular Protein is human CD40 1igand (CD40L) (UniProtKB-P29965 (CD40L_HUMAN)). CD40L is a cytokine that acts as a ligand to CD40/TNFRSF5. It costimulates T-cell proliferation and cytokine production. Its cross-linking on T-cells generates a costimulatory signal which enhances the production of IL4 and IL10 in conjunction with the TCR/CD3 1igation and CD28 costimulation. CD40L induces the activation of NF-kappa-B, as well as kinases MAPK8 and PAK2 in T-cells. It also induces tyrosine phosphorylation of isoform 3 of CD28. CD40L mediates B-cell proliferation in the absence of co-stimulus as well as IgE production in the presence of IL4, and is involved in immunoglobulin class switching.

The Protein Data Bank website provides the crystal structure of CD40L searchable by 1ALY (Karpusas, M., et al., Structure, 1995, 3, 1031-1039); as well as the crystal structure of CD40L bound to various compounds searchable by 3QD₆ (An, H.J., et al., J Biol Chem., 2011, 286, 11226-11235); and 6BRB (Karnell, J. L., et al., Sci Transl Med., 2019, 11 (489), 6584).

The expression of CD40L has been implicated in HIV-associated neurocognitive disorders and cardiovascular complications. Representative CD40L Targeting Ligands are provided in FIG. 1.

### Urokinase-type Plasminogen Activator (UPA)

In some embodiments, the Target Extracellular Protein is human urokinase-type plasminogen activator (UPA) (UniProtKB-P00749 (UROK_HUMAN)). Urokinase-type plasminogen activator (uPA), is a serine protease present in the blood and in the extracellular matrix of many tissues. The primary physiological substrate of this enzyme is plasminogen, which is an inactive form (zymogen) of the serine protease plasmin. Activation of plasmin triggers a proteolytic cascade that, depending on the physiological environment, participates in thrombolysis or extracellular matrix degradation. This cascade had been involved in vascular diseases and cancer progression. Elevated expression levels of urokinase and several other components of the plasminogen activation system are found to be correlated with tumor malignancy.

The Protein Data Bank website provides the crystal structure of UPA bound to various compounds searchable by 5ZA7, 5ZAJ, 5ZA8, 5ZA9, 5ZAE, 5ZAF, 5ZAG, 5ZAH, and 5ZC5 (Buckley, B. J. et al., J Med Chem., 2018, 61, 8299-8320); 5LHP, 5LHQ, 5LHR, and 5LHS (Kromann-Hansen, T. et al., Sci Rep., 2017, 7, 3385-3385); 2VNT (Fish, P. V. et al. J Med Chem., 2007, 50, 2341); 1OWD, IOWE, 1OWH, IOWI, 10WJ, and 1OWK (Wendt, M. D. et al., J Med Chem., 2004, 47, 303-324); 1SQA, 1SQO, and 1SQT (Wendt, M. D., et al., Bioorg Med Chem Lett., 2004, 14, 3063-3068); 1U6Q (Bruncko, M. et al., Bioorg Med Chem Lett., 2005, 15, 93-98); 30X7, 30Y5 and 30Y6 (Jiang, L. G. et al., J Mol Biol., 2011, 412, 235-250); 40S1, 40S2, 40S4, 40S5, 40S6 and 40S7 (Chen, S. et al., Nat Chem., 2014, 6, 1009-1016); 31G6 (West, C. W. et al., Bioorg Med Chem Lett., 2009, 19, 5712-5715); 4X0W and 4X1P (Jiang, L. et al., Int J Biochem Cell Biol., 2015, 62, 88-92); 4XIN, 4X1Q, 4X1R and 4X1S (Zhao, B. et al., PLOS One, 2014, 9, e115872-e115872); 5WXO and 5WXP (Jiang, L. et al., Biochim Biophys Acta., 2018, 1862, 2017-2023); 4MNV, 4MNW, 4MNX, and 4MNY (Chen, S., et al., Angew Chem Int Ed Engl., 2014, 53, 1602-1606); 4GLY (Chen, S., et al., J Am Chem Soc., 2013, 135, 6562-6569); 4JK5 and 4JK5 (Chen, S., et al., Chembiochem., 2013, 14, 1316-1322); 3QN7 (Angelini, A. et al., ACS Chem Biol., 2012, 7, 817-821); 2NWN (Zhao, G. et al., J Struct Biol., 2007, 160, 1-10); 6NMB (Wu, G. et al., Blood Adv., 2019, 3, 729-733); 1WOZ, 1W10, 1W11, 1W12, 1W13, and 1W14 (Zeslawska, E. et al., J Mol Biol., 2003, 328, 109); 4DVA (Jiang, L et al., Biochem J., 2013, 449, 161-166); 6A8G 6A8N (Wang, D. et al., J Med Chem., 2019, 62, 2172-2183); 2VIN, 2VIO, 2VIP, 2VIQ, 2VIV, and 2VIW (Frederickson, M. et al., J Med Chem., 2008, 51, 183); 1EJN (Speri, S., et al., Proc Natl Acad Sci USA, 2000, 97, 5113-5118); 3PB1 (Lin, Z. et al., J Biol Chem., 2011, 286, 7027-7032); 3U73 (Xu, X. et al., J Mol Biol., 2012, 416, 629-641); 1C₅W, 1C₅X, 1C₅Y and IC5Z (Katz, B. A., et al., Chem Biol., 2000, 7, 299-312); 5XG4 (Xue, G. et al., Food Funct., 2017, 8, 2437-2443); 5WXF (Jiang, L. et al., Biochim Biophys Acta., 2018, 1862, 2017-2023); 5WXS, 4ZKS, 5WXQ, 5WXT, 5YC6, 5YC7, 5ZIC, (Jiang, L. et al.); 4H₄₂ (Yu, H.Y. et al.,); 6AG3 and 6AG9 (Buckley, B. et al); 3KGP, 3KHV, 3KID, 3M61, 3MHW, and 3MWI (Jiang, L. G. et al.,); 4ZKN, 4ZKO and 4ZKR (Jiang, L. et al.); 208T, 208U, 208W (Zhao, G. et al.,); and 4FU7, 4FU8, 4FU9, 4FUB, 4FUC, 4FUD, 4FUE, 4FUF, 4FUG, 4FUH, 4FUI, and 4FUJ (Kang, Y. N. et al.).

Representative UPA Targeting Ligands are provided in FIG. 1. Additional UPA Targeting Ligands are provided in, for example, J Med Chem 38:1511-22 (1995), Bioorg Med Chem Lett 11:2253-6 (2001), Bioorg Med Chem Lett 14:3063-8 (2004), J Med Chem 52:3159-65 (2009), CSAR1: (2012), Bioorg Med Chem 22:3187-203 (2014), J Med Chem 50:2341-51 (2007), J Mol Biol 329:93-120 (2003), Bioorg Med Chem Lett2: 1399-1404 (1992), J Med Chem 35:4297-305 (1992), J Med Chem 35:4150-9 (1992), J Med Chem 49:5785-93 (2006), Bioorg Med Chem 23:3696-704 (2015), Bioorg Med Chem Lett 10:983-7 (2000), J Med Chem 49:5785-93 (2006), each of which is incorporated by reference herein.

### Plasminogen Activator, Tissue Type (TPA)

In some embodiments, the Target Extracellular Protein is human plasminogen activator, tissue type (TPA) (UniProtKB-P00750 (TPA_HUMAN)). TPA converts the abundant, but inactive, zymogen plasminogen to plasmin by hydrolyzing a single Arg-Val bond in plasminogen. By controlling plasmin-mediated proteolysis, it plays an important role in tissue remodeling and degradation, in cell migration and many other physiopathological events. TPA plays a direct role in facilitating neuronal migration. PLA has been shown activated in various cancers including oral malignancy.

The Protein Data Bank website provides the crystal structure of TPA searchable by 1VR1 (Dekker, R. J. et al., J Mol Biol., 1999, 293, 613-627); as well as the crystal structure of TPA bound to various compounds searchable by 1RTF (Lamba, D. et al., J Mol Biol., 1996, 258, 117-135); 1A5H (Renatus, M. et al., J Biol Chem., 1997, 272, 21713-21719); and 1BDA (Renatus, M. et al., EMBO J., 1997, 16, 4797-4805).

Representative TPA Targeting Ligands are provided in FIG. 1. Additional TPA Targeting Ligands are provided in, for example, Bioorg Med Chem Lett 15:4411-6 (2005), Bioorg Med Chem Lett 13:2781-4 (2003), Bioorg Med Chem Lett 6:2913-2918 (1996), J Med Chem 44:2753-71 (2001), J Med Chem 41:5445-56 (1999), Bioorg Med Chem Lett 12:3183-6 (2002), U.S. Pat. No. 10,118,930, J Biol Chem 285:7892-902 (2010), each of which is incorporated by reference herein.

### Plasminogen (PLG)

In some embodiments, the Target Extracellular Protein is human plasminogen (PLG) (UniProtKB-P00747 (PLMN_HUMAN)). PLG dissolves the fibrin of blood clots and acts as a proteolytic factor in a variety of other processes including embryonic development, tissue remodeling, tumor invasion, and inflammation. It activates the urokinase-type plasminogen activator, collagenases and several complement zymogens, such as C1 and C5. Its role in tissue remodeling and tumor invasion may be modulated by CSPG4.

The Protein Data Bank website provides the crystal structure of PLG searchable by 1DDJ (Wang, X. et al., J.Mol.Biol., 2000, 295, 903-914); and 4DUR and 4DUU (Law, R. H. P., et al., Cell Rep., 2012, 1, 185-190).

Representative PLG Targeting Ligands are provided in FIG. 1. Additional PLG Targeting Ligands are provided in, for example, J Med Chem 35:4297-305 (1992), J Med Chem 38:1511-22 (1995), J Med Chem 56:820-31 (2013), U.S. Pat. Nos. 8,598,206, 8,921,319, J Med Chem 55:1171-80 (2012), Bioorg Med Chem Lett 12:3183-6 (2002), Bioorg Med Chem 23:3696-704 (2015), Bioorg Med Chem Lett 13:723-8 (2003), Bioorg Med Chem Lett 7:331-336 (1997), each of which is incorporated by reference herein.

### Plasminogen Activator Inhibitor-1 (PAI-1)

In some embodiments, the Target Extracellular Protein is human plasminogen activator inhibitor 1 (PAI-1) (UniProtKB-P05121 (PAI1_HUMAN)). PAI-1 is a serine protease inhibitor, and a primary inhibitor of tissue-type plasminogen activator (PLAT) and urokinase-type plasminogen activator (PLAU). As PLAU inhibitor, it is involved in the regulation of cell adhesion and spreading, and acts as a regulator of cell migration, independently of its role as protease inhibitor. Overexpression of PAI-1 favors angiogenesis, metastasis, and poor prognosis in tumors, including, but not limited to, oral cancers and breast cancers.

The Protein Data Bank website provides the crystal structure of PAI-1 searchable by 3Q02 and 3Q03 (Jensen, J. K. et al., J Biol Chem., 2011, 286, 29709-29717); 1B3K (Sharp, A. M. et al., Structure, 1999, 7, 111-118); 1C5G (Tucker, H. M. et al., Nat Struct Biol., 1995, 2, 442-445); 1DVM (Stout, T. J. et al., Biochemistry, 2000, 39, 8460-8469); and 3UT3 (Lin, Z. H. et al.,); as well as the crystal structure of PAI-1 bound to various compounds searchable by 4AQH (Fjellstrom, O. et al., J Biol Chem., 2013, 288, 873); 3R⁴L (Jankun, J. et al., Int J Mol Med., 2012, 29 61-64); 1A7C (Xue, Y., et al., Structure, 1998, 6, 627-636); 10C0 (Zhou, A. et al., Nat Struct Biol., 2003, 10, 541); 618S (Vousden, K. A. et al., Sci Rep., 2019, 9, 1605-1605); 4G80 and 4G8R (Li, S.H. et al., Proc Natl Acad Sci USA, 2013, 110, E4941-E4949); 6GWQ, 6GWN and 6GWP (Sillen, M. et al., J Thromb Haemost, 2019); and 41CO (Hong, Z. B. et al.,).

Representative PAI-1 Targeting Ligands are provided in FIG. 1. Additional PAI-1 Targeting Ligands are provided in, for example, J Biol Chem 285:7892-902 (2010), U.S. Pat. No. 9,120,744, Bioorg Med Chem Lett 13:3361-5 (2003), Bioorg Med Chem Lett 12:1063-6 (2002), Bioorg Med Chem Lett 13:1705-8 (2003), Bioorg Med Chem Lett 11:2589-92 (2001), U.S. Pat. No. 9,718,760, each of which is incorporated by reference herein.

### Placenta Growth Factor (PIGF)

In some embodiments, the Target Extracellular Protein is human placental growth factor (PGF) (UniProtKB-P49763 (PLGF_HUMAN)). PGF is growth factor active in angiogenesis and endothelial cell growth, stimulating their proliferation and migration. It binds to the receptor FLT1/VEGFR-1. Isoform PIGF-2 binds NRP1/neuropilin-1 and NRP2/neuropilin-2 in a heparin-dependent manner. PGF also promotes cell tumor growth, and has been implicated in age-related macular degeneration (AMD) and choroidal neovascularization (CNV).

The Protein Data Bank website provides the crystal structure of PIGF searchable by 1FZV (Iyer, S. et al., J Biol Chem., 2001, 276, 12153-12161); as well as the crystal structure of PIGF bound to various compounds searchable by 1RV6 (Christinger, H. W., J Biol Chem., 2004, 279, 10382-10388). Additionally, De Falco provides insight into the discovery and biological activity of placenta growth factor (De Falco, Exp Mol Med., 2012, 44, 1-9).

Representative PGF Targeting Ligands are provided in FIG. 1. Additional PGF Targeting Ligands are provided in, for example, J Med Chem 54:1256-65 (2011), J Nat Prod 76:29-35 (2013), each of which is incorporated by reference herein.

### Phospholipase A2, Group IB (PA21B)

In some embodiments, the Target Extracellular Protein is human phospholipase A2, Group IB (PA21B) (UniProtKB-P04054 (PA21B_HUMAN)). PA21B cleaves phospholipids preferentially at the sn-2 position, liberating free fatty acids and lysophospholipids. PA21B has been implicated in a number of diseases, including cardiovascular diseases, atherosclerosis, immune disorders and cancer.

The Protein Data Bank website provides the crystal structure of PA21B searchable by 3FVJ and 3FVI (Pan, Y. H. et al., Biochim.Biophys.Acta., 2010, 1804, 1443-1448).

Representative PA21B Targeting Ligands are provided in FIG. 1. Additional PA21B Targeting Ligands are provided in, for example, J Med Chem 39:3636-58 (1996), Chembiochem 4:181-5 (2003), J Med Chem 39:5159-75 (1997), J Med Chem 51:4708-14 (2008), each of which is incorporated by reference herein.

### Phospholipase A2, Group IIA (PA2GA)

In some embodiments, the Target Extracellular Protein is human phospholipase A2, Group IIA (PA2GA) (UniProtKB-P04054 (PA21B_HUMAN)). PA2GA catalyzes the calcium-dependent hydrolysis of the 2-acyl groups in 3-sn-phosphoglycerides. It is thought to participate in the regulation of phospholipid metabolism in biomembranes including eicosanoid biosynthesis. Independent of its catalytic activity, it also acts as a ligand for integrins. PA2GA Induces cell proliferation in an integrin-dependent manner. PA2GA has been implicated in a number of diseases, including cardiovascular diseases, atherosclerosis, immune disorders, and cancer.

The Protein Data Bank website provides the crystal structure of PA2GA bound to various compounds searchable by 2ARM and 1SV3 (Singh, N. et al., Proteins, 2006, 64, 89-100); 5G3M and 5G3N (Giordanetto, F., et al. ACS Med Chem Lett., 2016, 7, 884); 1KQU (Jansford, K. A., et al., Chembiochem., 2003, 4,181-185); and 1ZYX (Singh, N. et al.,). Additionally, Singh et al., provides insight into the crystal structure of the complexes of a group IIA phospholipase A2 with two natural anti-inflammatory agents, anisic acid, and atropine reveal a similar mode of binding (Singh, N. et al., Proteins, 2006, 64 (1): 89-100); and Kitadokoro et al also provides insight into the crystal structure of human secretory phospholipase A2-IIA complex with the potent indolizine inhibitor 120-1032 (Kitadokoro, K. et al., J Biochem., 1998, 123 (4), 619-23).

Representative PA2GA Targeting Ligands are provided in FIG. 1. Additional PA2GA Targeting Ligands are provided in, for example, J Med Chem 48:893-6 (2005), J Med Chem 39:5159-75 (1997), each of which is incorporated by reference herein.

### Factor B

In some embodiments, the Target Extracellular Protein is human Complement factor B (UniProtKB-P00751 (CFAB_HUMAN)). Complement factor B, which is part of the alternate pathway of the complement system, is cleaved by factor D into 2 fragments: Ba and Bb. Bb, a serine protease, then combines with complement factor 3b to generate the C3 or C5 convertase. It has also been implicated in proliferation and differentiation of preactivated B-lymphocytes, rapid spreading of peripheral blood monocytes, stimulation of lymphocyte blastogenesis and lysis of erythrocytes. Ba inhibits the proliferation of preactivated B-lymphocytes.

The Protein Data Bank website provides the crystal structure of Complement Factor B searchable by 20K5 (Milder, F. J., et al., Nat Struct Mol Bio 2007, 14, 224-228); as well as the crystal structure of Complement factor B bound to various compounds searchable by 6QSW, 6QSX, and 6RAV (Schubart, A., et al., Proc Natl Acad Sci 2019, 116, 7926-7931); 6T8U, 6T8W, and 6T8V (Mainolfi, N., et al, J Med Chem 2020, 63, 5697-5722); and 7JTN (Xu, X., et al., J Immunol 2021, 206, doi: 10.4049/jimmunol.2001260).

Representative Complement Factor B Targeting Ligands are provided in FIG. 5. Additional Complement Factor B Targeting Ligands are provided in, for example, U.S. Pat. No. 9,682,968B2, U.S. Pat. No. 9,475,806B2, U.S. Pat. No. 9,452,990B2, Proc Natl Acad Sci 116:7926-7931 (2019), J Med Chem 52:6042-6052 (2009), and J Med Chem 63:5697-5722 (2020), each of which is incorporated by reference herein.

In certain embodiments the Extracellular Targeting Ligand is selected from:

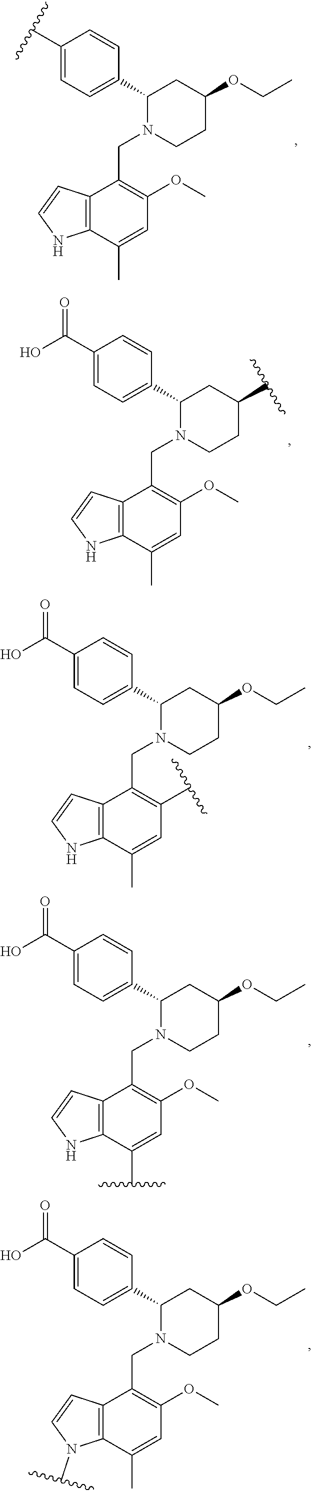 [SMILES: *c1ccc([C@@H]2C[C@@H](OCC)CCN2Cc2c(OC)cc(C)c3[nH]ccc23)cc1 ; *[C@H]1CCN(Cc2c(OC)cc(C)c3[nH]ccc23)[C@H](c2ccc(C(=O)O)cc2)C1 ; *c1cc(C)c2[nH]ccc2c1CN1CC[C@H](OCC)C[C@H]1c1ccc(C(=O)O)cc1 ; *c1cc(OC)c(CN2CC[C@H](OCC)C[C@H]2c2ccc(C(=O)O)cc2)c2cc[nH]c12 ; *n1ccc2c(CN3CC[C@H](OCC)C[C@H]3c3ccc(C(=O)O)cc3)c(OC)cc(C)c21]

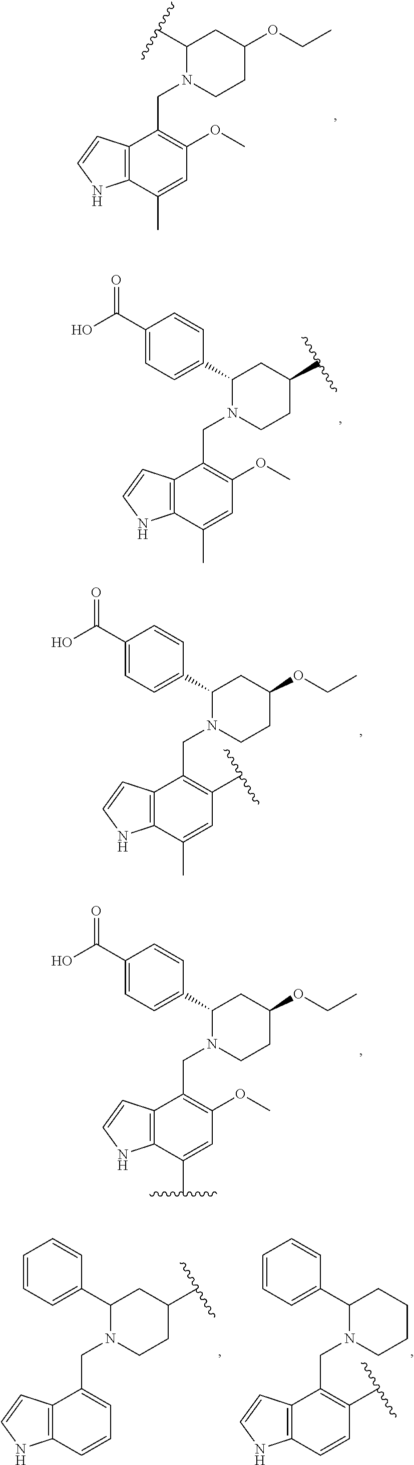 [SMILES: *C1CC(OCC)CCN1Cc1c(OC)cc(C)c2[nH]ccc12 ; *[C@H]1CCN(Cc2c(OC)cc(C)c3[nH]ccc23)[C@H](c2ccc(C(=O)O)cc2)C1 ; *c1cc(C)c2[nH]ccc2c1CN1CC[C@H](OCC)C[C@H]1c1ccc(C(=O)O)cc1 ; *c1cc(OC)c(CN2CC[C@H](OCC)C[C@H]2c2ccc(C(=O)O)cc2)c2cc[nH]c12 ; *C1CCN(Cc2cccc3[nH]ccc23)C(c2ccccc2)C1 ; *c1ccc2[nH]ccc2c1CN1CCCCC1c1ccccc1]

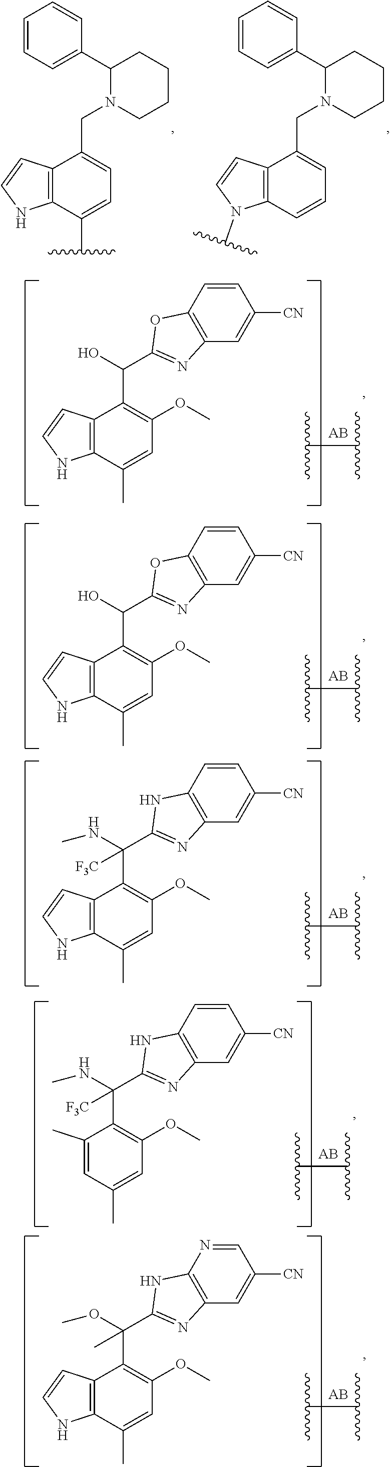

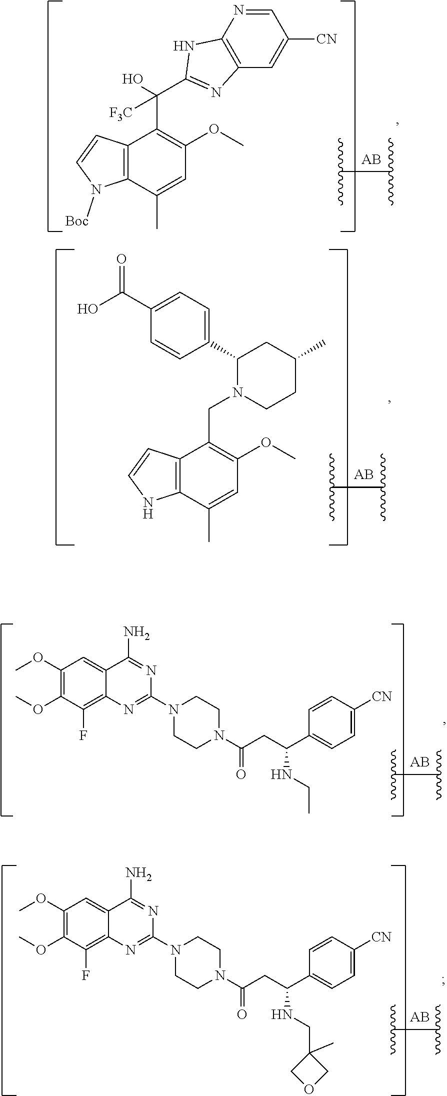

each of which is optionally substituted with 1, 2, 3, or 4 substituents independently selected from R²¹.

In certain embodiments the Factor B Targeting Ligand is selected from a ligand described in: Mainolfi, N. et. al. Discovery of 4-((2 S,4 S)-4-Ethoxy-1-((5-Methoxy-7-Methyl-1 H-Indol-4-Y1) Methyl) Piperidin-2-Y1) Benzoic Acid (LNP023), a Factor B Inhibitor Specifically Designed To Be Applicable to Treating a Diverse Array of Complement Mediated Diseases. J. Med. Chem. 2020, 63 (11), 5697-5722; WO2020/016749; WO2018/005552; WO2013/192345; or WO2015/009616.

In certain embodiments the factor B Targeting Ligand-linker is selected from:

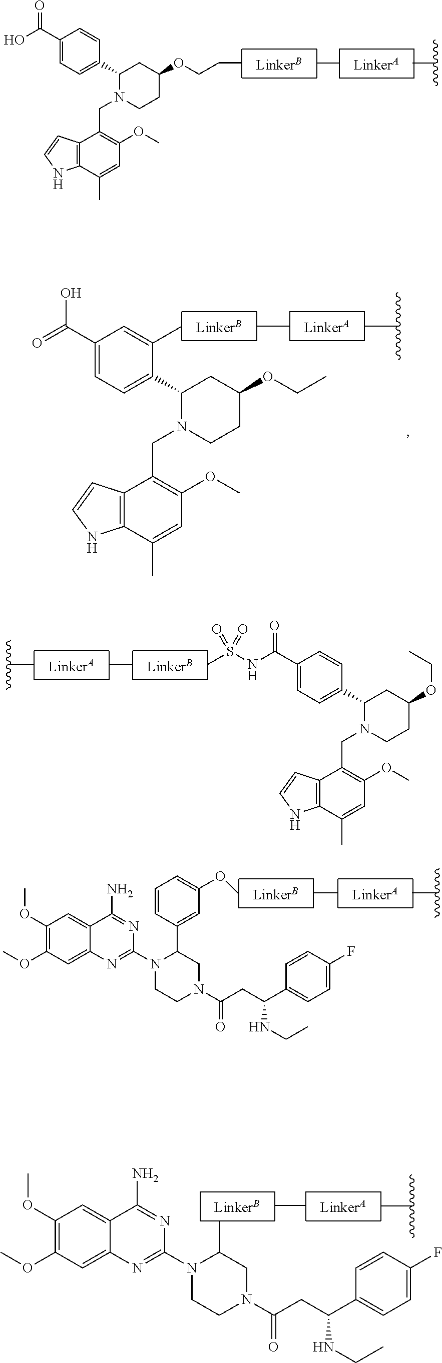

In certain embodiments the compound of the present invention is selected from the following compounds or a bi- or tri-dentate version thereof:

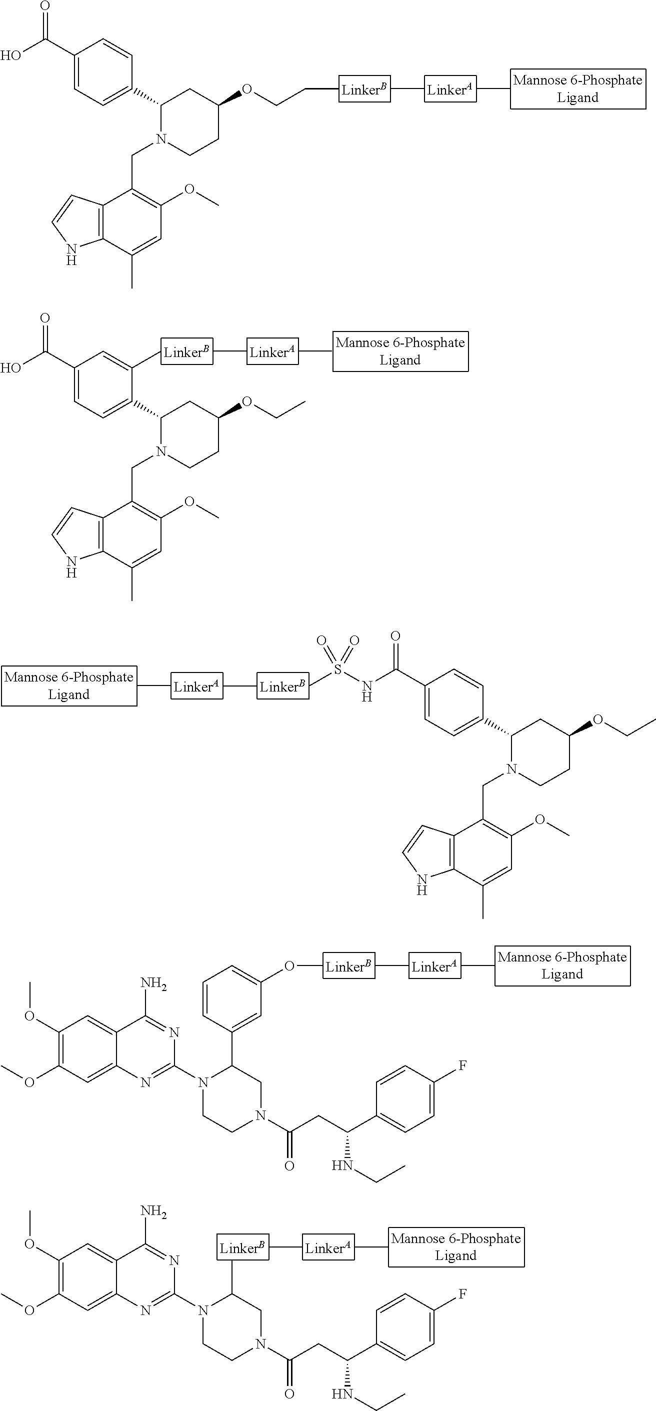

In certain embodiments the Factor B Targeting Ligand is selected from a ligand described in WO 2013/164802; WO 2014/143638; WO 2015/066241; or WO 2019/043609.

### Factor D

In some embodiments, the Target Extracellular Protein is human Complement factor D (UniProtKB-P00746 (CFAD_HUMAN)). Factor D cleaves factor B when the latter is complexed with factor C3b, activating the C3bbb complex, which then becomes the C3 convertase of the alternate pathway. Its function is homologous to that of C1s in the classical pathway.

The Protein Data Bank website provides the crystal structure of Complement factor D bound to various compounds searchable by 6FTZ, 6FUT, 6FUH, 6FUG, 6FUJ, and 6FUI (Vulpetti, A., et al., ACS Med Chem Lett 2018, 9, 490-495); 5TCA and 5TCC (Yang, C. Y., et al., ACS Med Chem Lett 2016, 7, 1092-1096); 5MT4 (Vulpetti, A., et al., J Med Chem 2017, 60, 1946-1958); 1DFP (Cole, L. B., et al., Acta Crystallogr D Biol Crystallogr 1997, 53, 143-150); 1DIC (Cole, L. B., et al., Acta Crystallogr D Biol Crystallogr 1998, 54, 711-717); 6QMR and 6QMT (Karki, R. G., et al., J Med Chem 2019, 62, 4656-4668).

Representative Complement factor D Targeting Ligands are provided in FIG. 6. Additional Complement Factor D Targeting Ligands are provided in, for example, J Med Chem 60:5717-5735 (2017), Nat Chem Biol 12:1105-1110 (2016), U.S. Pat. No. 9,598,446B2, U.S. Pat. No. 9,643,986B2, US patent U.S. Pat. No. 9,663,543B2 US patent U.S. Pat. No. 9,695,205B2, U.S. Pat. No. 9,732,103B2, U.S. Pat. No. 9,732,104B2, U.S. Pat. No. 9,758,537B2, U.S. Pat. No. 9,796,741B2, U.S. Pat. No. 9,828,396B2, U.S. Pat. No. 10,000,516B2, U.S. Pat. No. 10,005,802B2, U.S. Pat. No. 10,011,612B2, U.S. Pat. No. 10,081,645B2, U.S. Pat. No. 10,087,203B2, U.S. Pat. No. 10,092,584B2, U.S. Pat. No. 10,100,072B2, U.S. Pat. No. 10,106,563B2, U.S. Pat. No. 10,138,225B2, U.S. Pat. No. 10,189,869B2, U.S. Pat. No. 10,253,053B2, U.S. Pat. No. 10,287,301B2, U.S. Pat. No. 10,301,336B2, U.S. Pat. No. 10,370,394B2, U.S. Pat. No. 10,385,097B2, U.S. Pat. No. 10,428,094B2, U.S. Pat. No. 10,428,095B2, U.S. Pat. No. 10,464,956B2, U.S. Pat. No. 10,550,140B2, U.S. Pat. No. 10,660,876B2, U.S. Pat. No. 10,662,175B2, US U.S. Pat. No. 10,689,409B2, U.S. Pat. No. 10,807,952B2, U.S. Pat. No. 10,822,352B2, U.S. Pat. No. 9,464,081B2, and Hematological 102:466-475 (2017), each of which is incorporated by reference herein.

In certain embodiments the Extracellular Targeting Ligand is selected from:

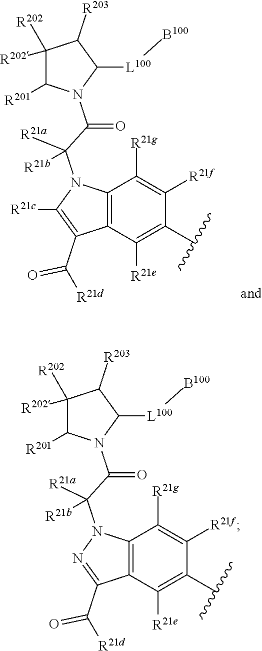

wherein

R^{21a}, R^{21b}, R^{21c}, R^{21d}, R²¹e, R^{21f}, and R^{21g} are independently at each occurrence selected from the group consisting of hydrogen, alkyl, alkenyl, alkynyl, F, Cl, Br, I, hydroxyl, alkoxy, azide, amino, cyano, —NR⁶R⁷, —NR⁸SO₂R³, —NR⁸S(O)R³, haloalkyl, aryl, heteroaryl, heterocyclyl, —SR³, —C(O)OR³, —C(O)NR⁶NR⁷, —OR³, and heterocycle;
R²⁰¹, R²⁰², R²⁰²′, and R²⁰³ are independently selected from hydrogen, halogen, hydroxyl, nitro, cyano, amino, C₁-C₆alkyl, C₂-C₅alkenyl, C₁-C₆alkoxy, C₂-C₅alkynyl, C₂-C₅alkanoyl, C₁-C₆thioalkyl, hydroxyC₁-C₆alkyl, aminoC₁-C₆alkyl, —C₀-C₄alkylNR⁹R¹⁰, —C(O)OR⁹, —OC(O)R⁹, —NR⁹C(O)R¹⁰, —C(O)NR⁹R¹⁰, —OC(O)NR⁹R¹⁰, —O (heteroaryl), —NR⁹C(O)OR¹⁰, C₁-C₇haloalkyl, —C₀-C₄alkyl(C₃-C₇cycloalkyl) and —O—C₀-C₄alkyl(C₃-C₇cycloalkyl), and C₁-C₇haloalkoxy, where R²⁰⁹ and R²¹⁰ are independently chosen at each occurrence from hydrogen, C₁-C₆alkyl, and (C₃-C₇cycloalkyl)C₀-C₄alkyl;
or R²⁰² and R²⁰² may be taken together to form a 3- to 6-membered spiro ring optionally substituted with 1 or more substituents independently chosen from halogen, hydroxyl, cyano, —COOH, C₁-C₄alkyl (including in particular methyl), C₂-C₄alkenyl, C₂-C₄alkynyl, C₁-C₄alkoxy, C₂-C₄alkanoyl, hydroxyC₁-C₄alkyl, and di-C₁-C₄alkylamino)C₀-C₄alkyl, (mono-—C₀-C₄alkyl(C₃-C₇cycloalkyl), —O—C₀-C₄alkyl(C₃-C₇cycloalkyl), C₁-C₇haloalkyl, and C₁-C₇haloalkoxy.
or R²⁰¹ and R²⁰² may be taken together to form a 3-membered carbocyclic ring, optionally substituted with 1, 2, or 3 substituents selected from R²¹.
or R²⁰¹ and R²⁰² may be taken together to form a 4- to 6-membered carbocyclic ring or a 4- to 6-membered heterocyclic ring containing 1 or 2 heteroatoms independently chosen from N, O, and S, optionally substituted with 1, 2, or 3 substituents selected from R²¹.

R²⁰² and R²⁰³ may be taken together to form a 3- to 6-membered carbocyclic ring or a 3- to 6-membered heterocyclic ring optionally substituted with 1, 2, or 3 substituents selected from R²¹.
L¹⁰⁰ is selected from

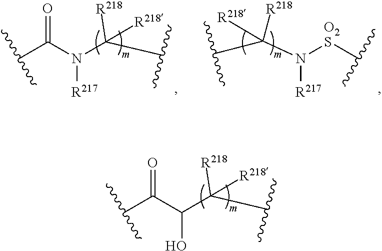

wherein R²¹⁷ is hydrogen or C₁-C₆alkyl and R²¹⁸ and R²¹⁸′ are independently chosen from hydrogen, halogen, hydroxymethyl, and methyl; and m is 0, 1, 2, or 3;
B¹⁰⁰ is a cycloalkyl, heterocycle group having 1, 2, 3, or 4 heteroatoms independently selected from N, O, and S, a C₂-C₅alkenyl, C₂-C₆alkynyl group, —(C₀-C₄alkyl) (aryl), —(C₀-C₄alkyl) (heteroaryl), or —(C₀-C₄alkyl) (biphenyl), each of which is optionally substituted with 1, 2, 3, or 4 substituents independently selected from R²¹.

In certain embodiments the Extracellular Targeting Ligand is selected from:

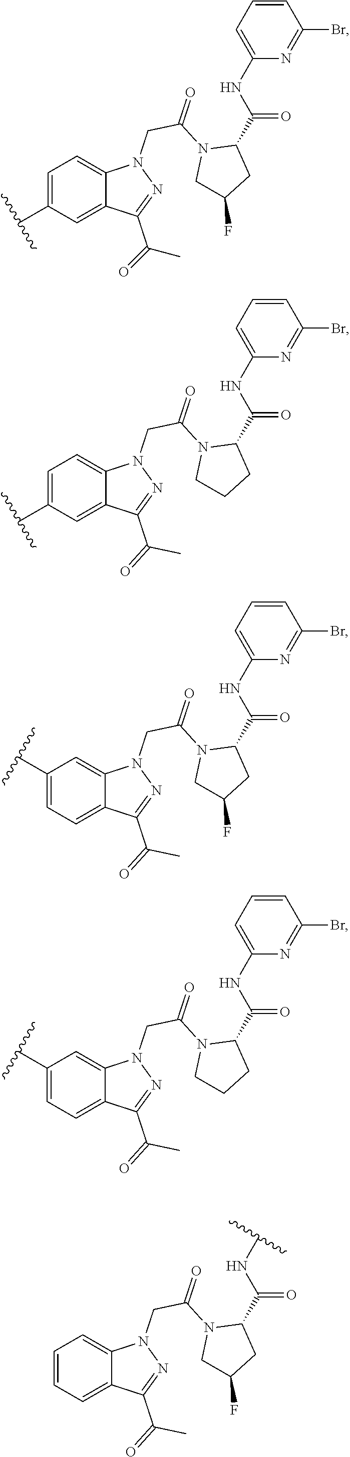 [SMILES: *c1ccc2c(c1)c(C(C)=O)nn2CC(=O)N1C[C@H](F)C[C@H]1C(=O)Nc1cccc(Br)n1 ; *c1ccc2c(c1)c(C(C)=O)nn2CC(=O)N1CCC[C@H]1C(=O)Nc1cccc(Br)n1 ; *c1ccc2c(C(C)=O)nn(CC(=O)N3C[C@H](F)C[C@H]3C(=O)Nc3cccc(Br)n3)c2c1 ; *c1ccc2c(C(C)=O)nn(CC(=O)N3CCC[C@H]3C(=O)Nc3cccc(Br)n3)c2c1 ; *NC(=O)[C@@H]1C[C@@H](F)CN1C(=O)Cn1nc(C(C)=O)c2ccccc21]

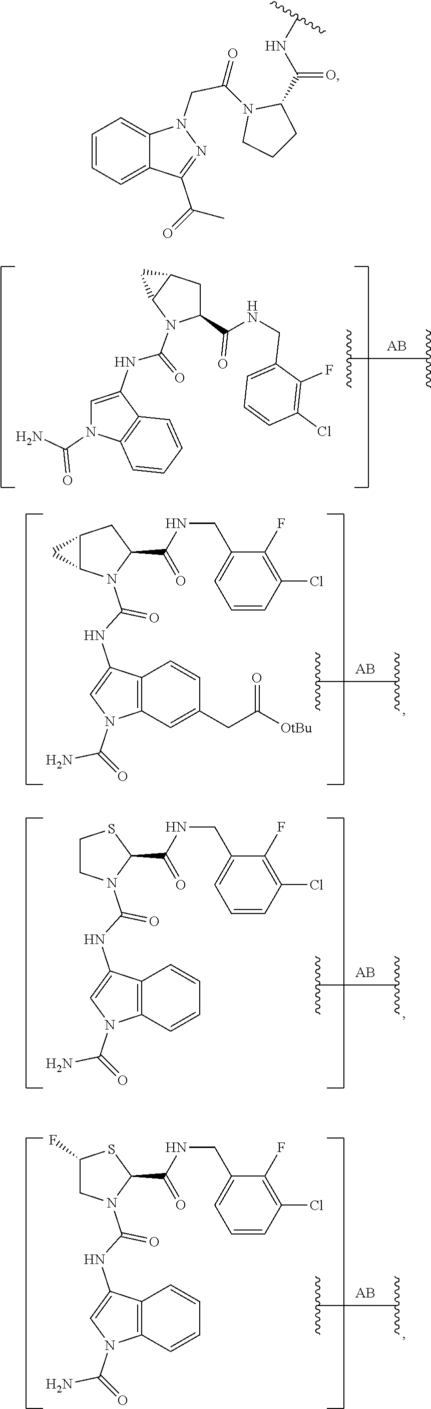

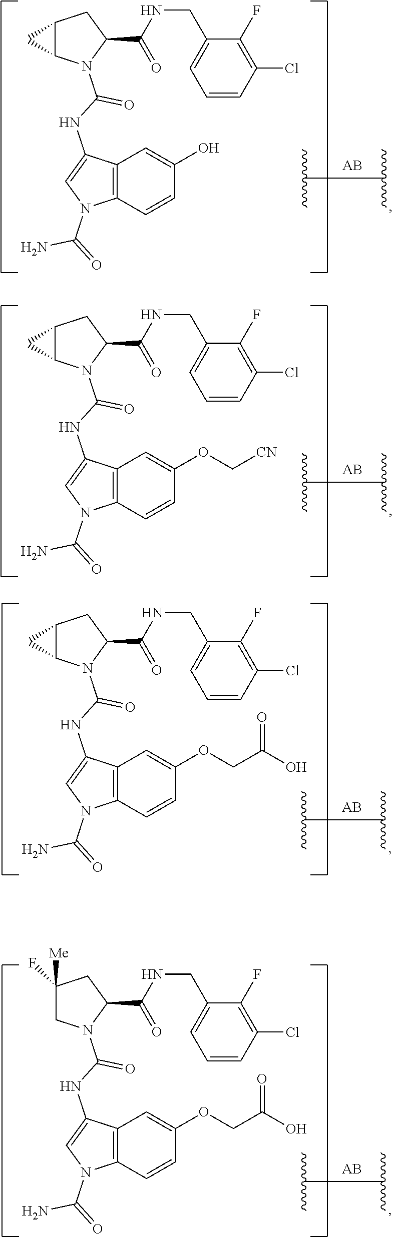

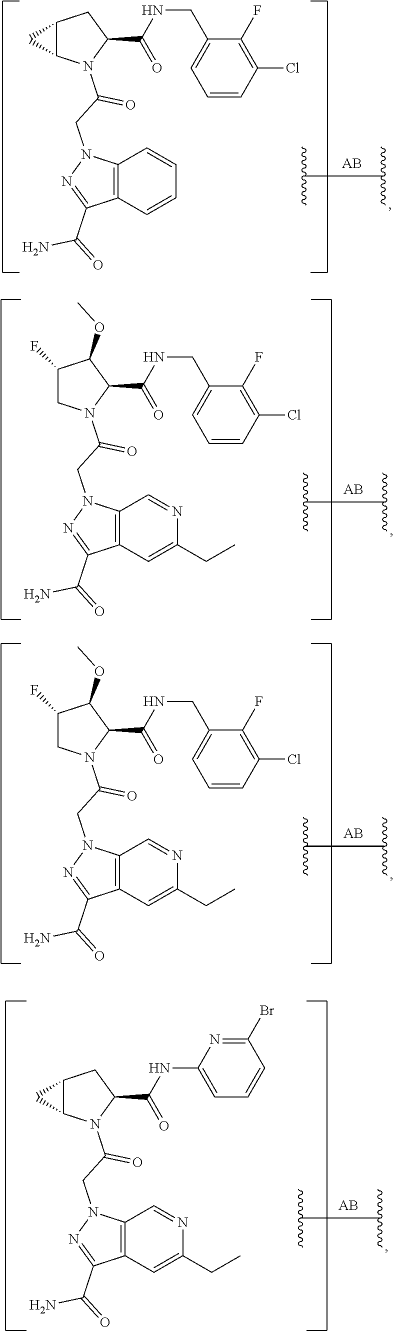

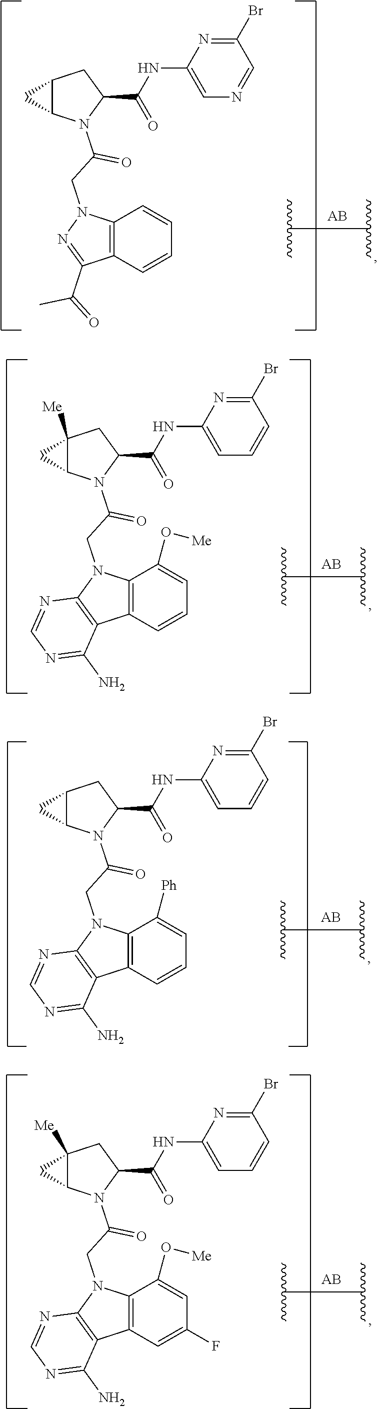

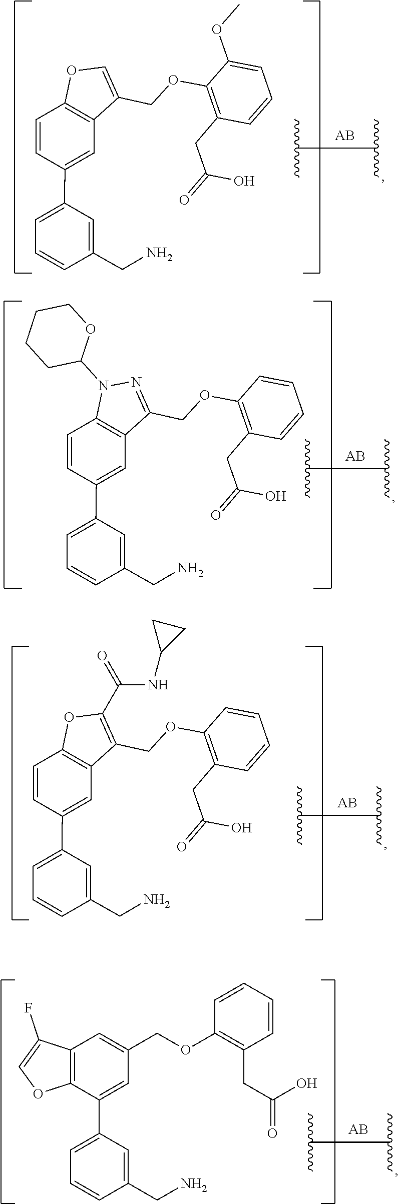

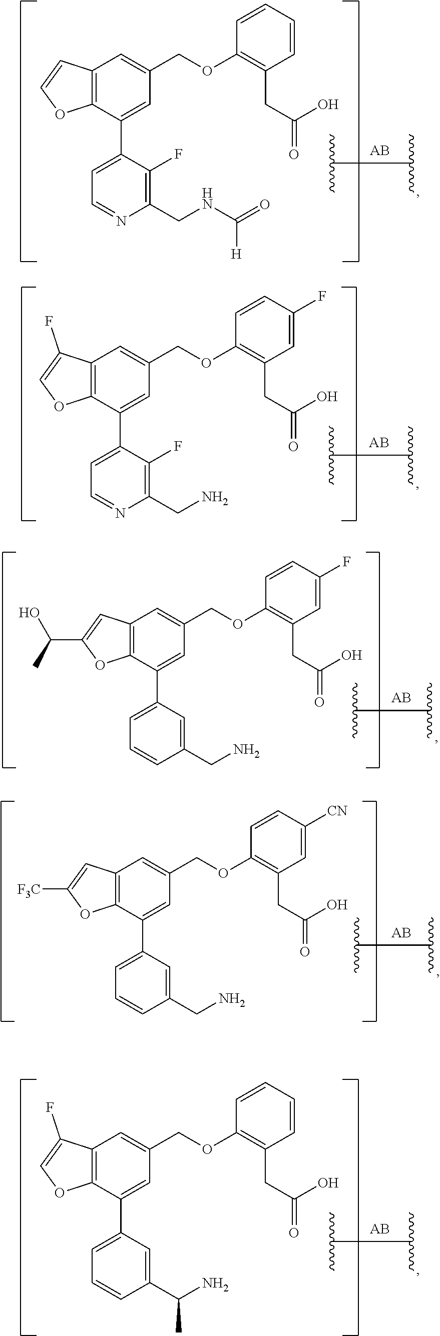

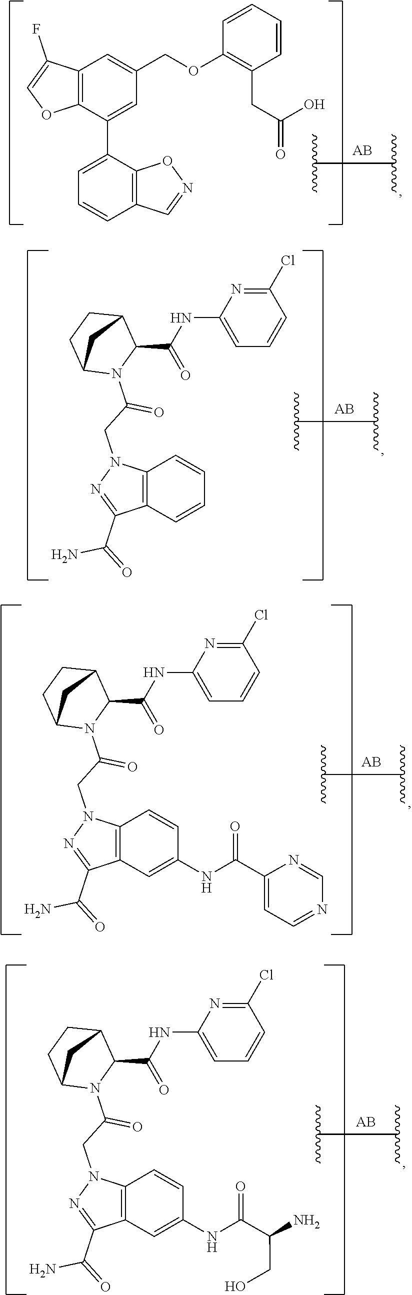

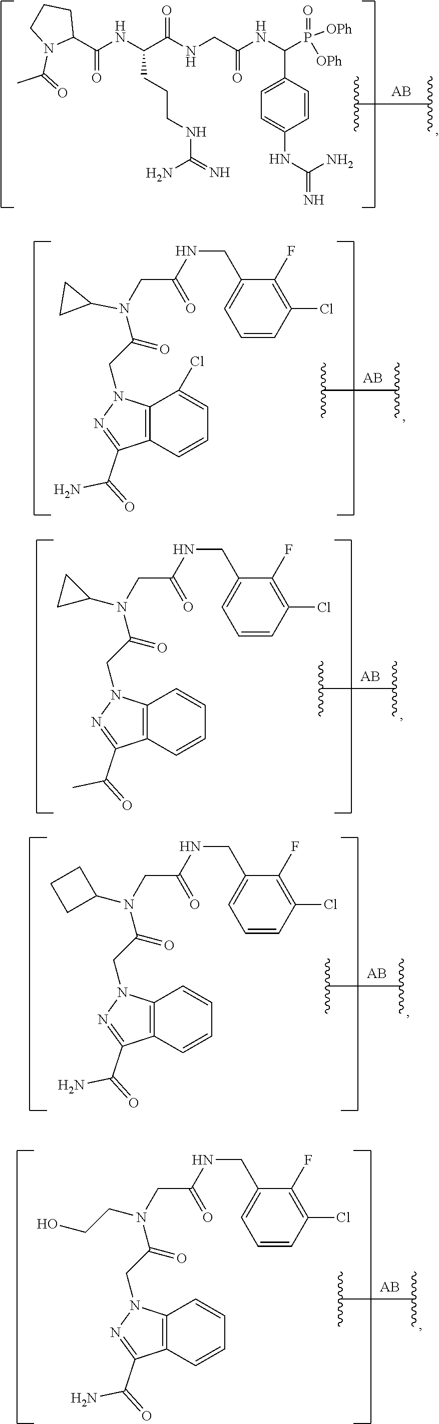

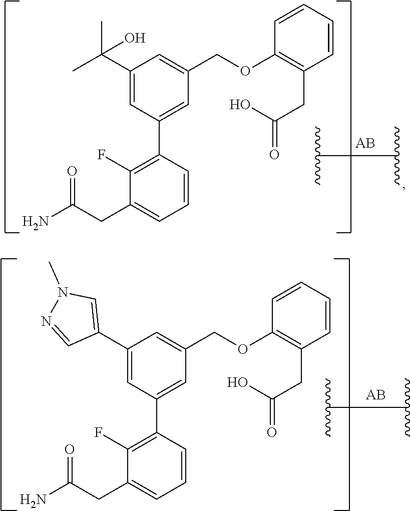

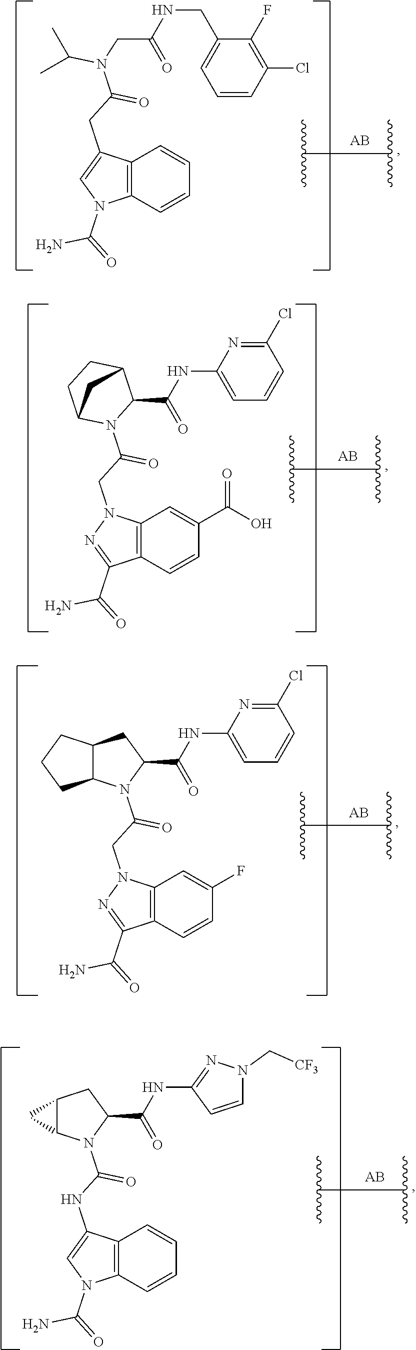

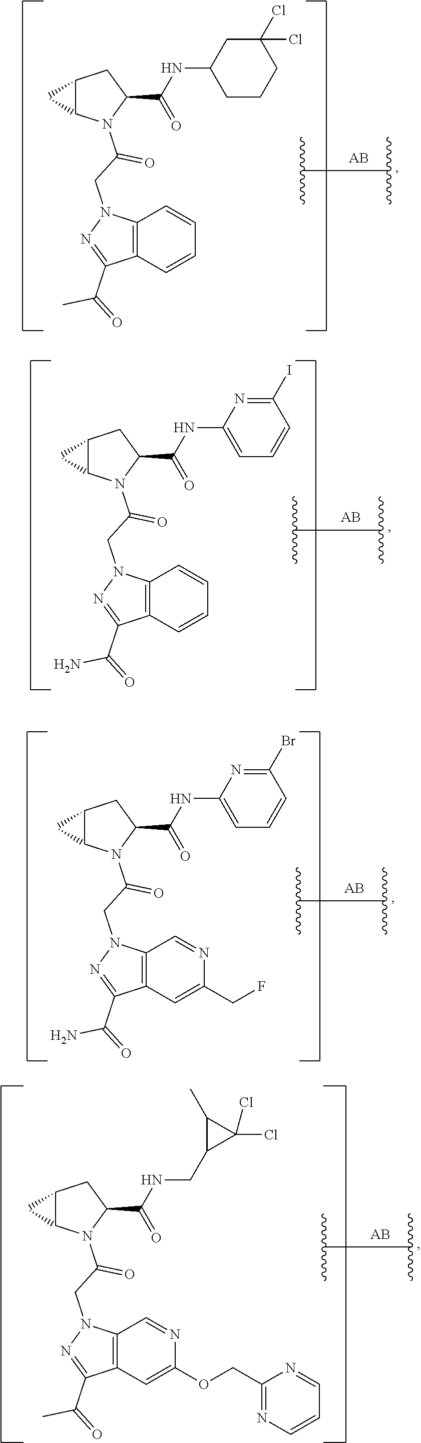

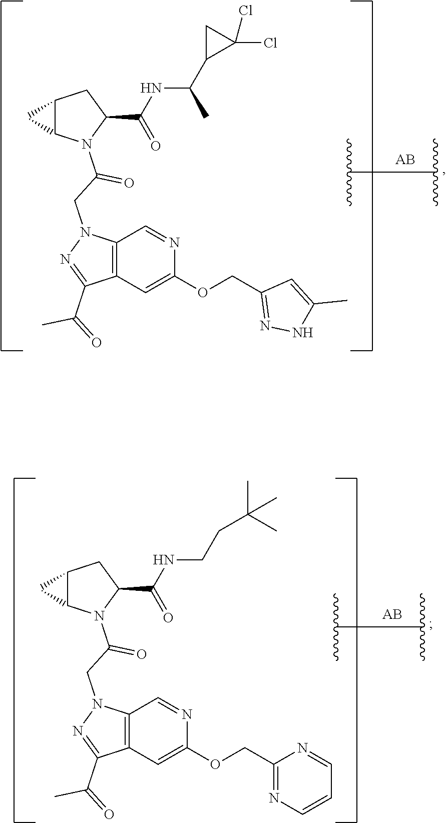

each of which is optionally substituted with 1, 2, 3, or 4 substituents independently selected from R²¹ and is attached to the linker at a location allowed by valence.

In certain embodiments the Factor D Targeting Ligand is selected from a ligand described in: U.S. Patent 9,796,74; U.S. Pat. No. 10,011,612; WO2018/160889; WO2019/195720; WO2019/057946; Karki, R. G. et al. Design, Synthesis, and Preclinical Characterization of Selective Factor D Inhibitors Targeting the Alternative Complement Pathway. J. Med. Chem. 2019, 62 (9), 4656-4668; or Belanger, D. B. et al.; WO2015/009977.

In certain embodiments the Factor B Targeting Ligand is selected from a ligand described in WO2012093101A1; WO2014002051A2; WO2014002052A1; WO2014002053A1; WO2014002054A1; WO2014002057A1; WO2014002058A2; WO2014009833A2; WO2015130784A1; WO2015130795A1; WO2015130806A1; WO2015130830A1; WO2015130838A1; WO2015130842A2; WO2015130845A1; WO2015130854A1; WO2016088082A1; WO2016203313A1; WO2017035348A1; WO2017035349A1; WO2017035351A1; WO2017035352A1; WO2017035353A1; WO2017035355A1; WO2017035357A1; WO2017035360A1; WO2017035361A1; WO2017035401A1; WO2017035405A1; WO2017035408A1; WO2017035409A1; WO2017035411A1; WO2017035413A2; WO2017035415A1; WO2017035417A1; WO2017035418A1; WO2017098328A2; WO2017136395A1; WO2018015818A2; WO2018024818A1; WO2018160889A1; WO2018160891A1; WO2018160892A1; WO2018229543A2; WO2019028284A1; WO2019195720A1; WO2020041301A1; WO2020051532A2; WO2020051538A1; WO2020131974A1; WO2020198062A1; WO2021021909A1; WO2021072156A1; WO2021072198A1; WO2021168320A1; WO2021202977A1; or WO2021231470A1.

In certain embodiments the complement factor D targeting ligand-linker- is selected from:

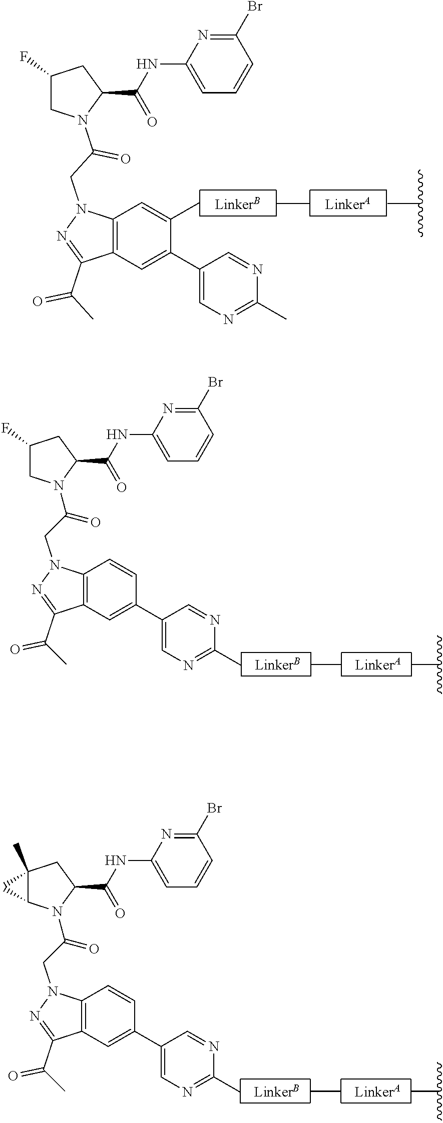

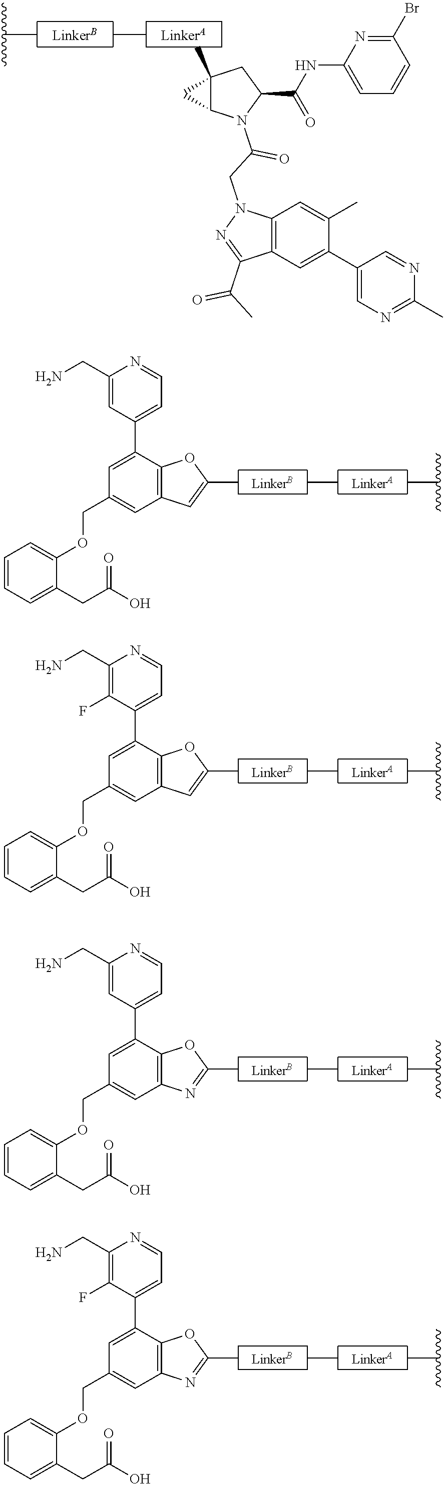

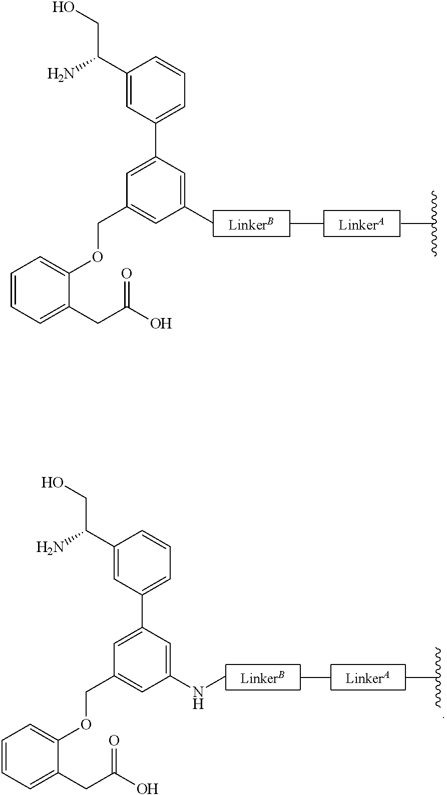

In certain embodiments the compound of the present invention is selected from the following compounds or a bi- or tri-dentate version thereof:

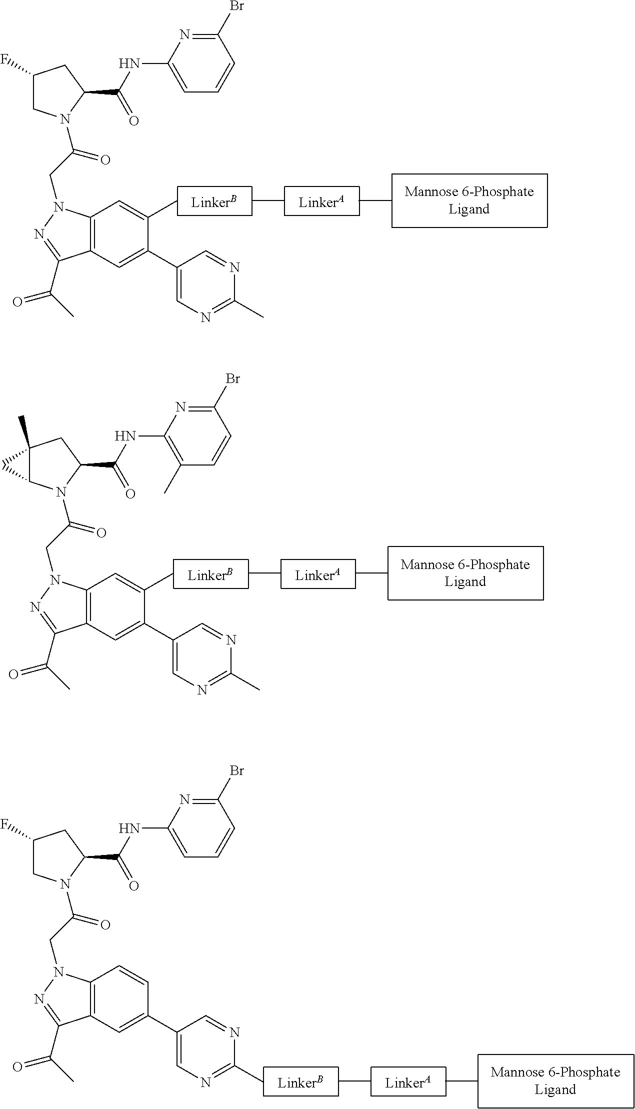

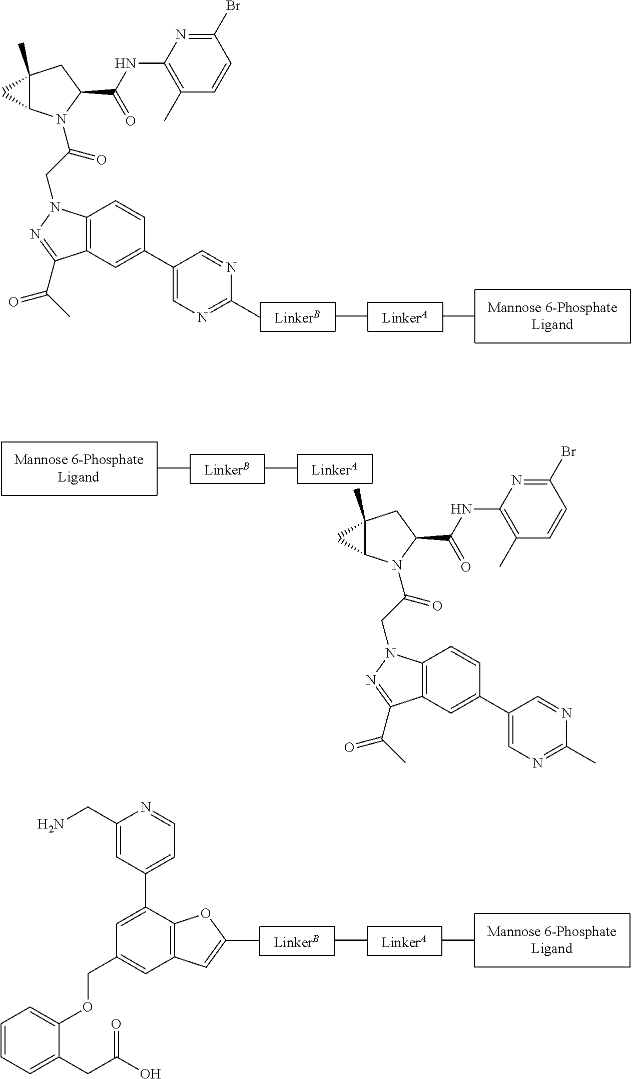

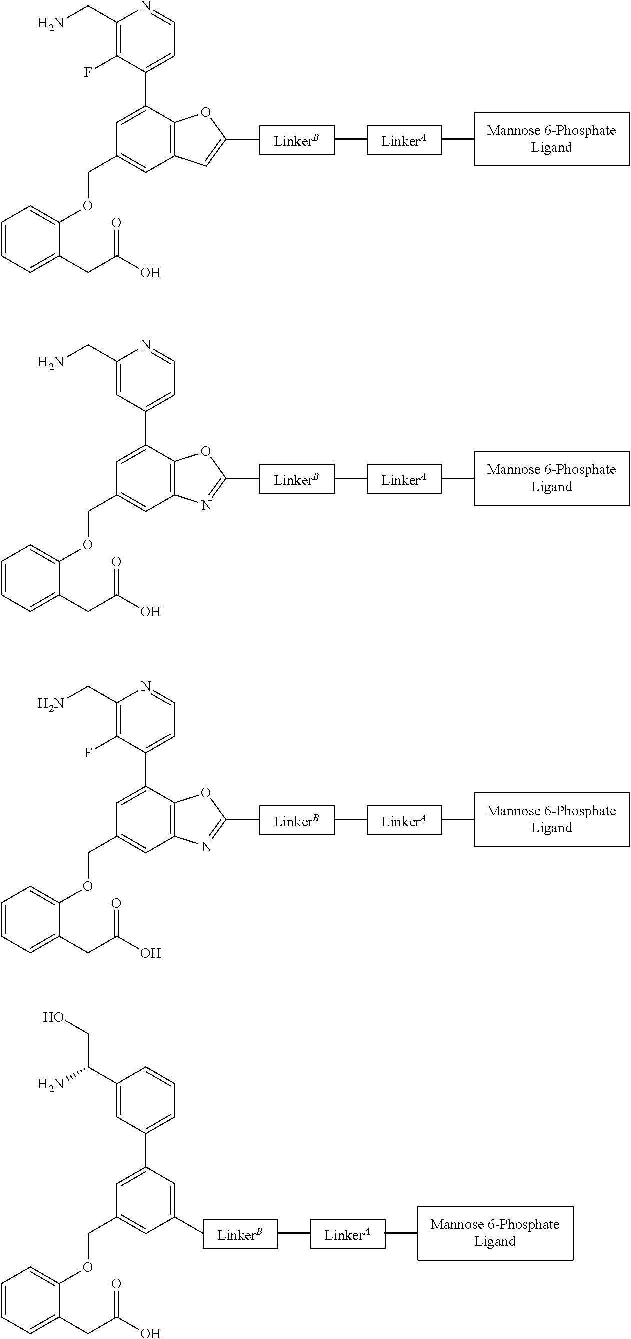

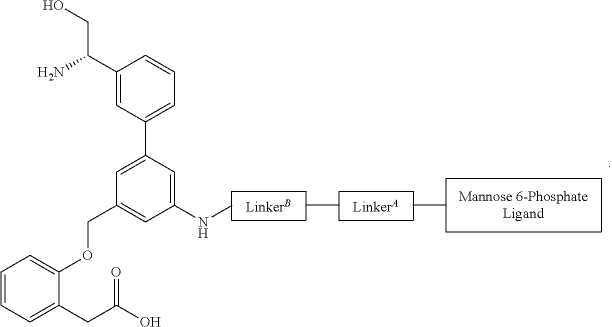

In certain embodiments the Factor D Targeting Ligand is selected from:

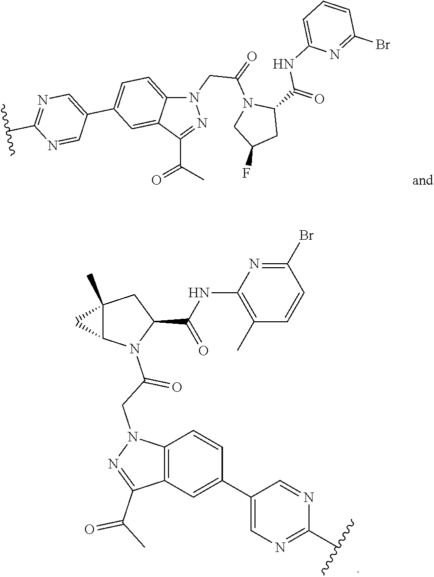 [SMILES: *c1ncc(-c2ccc3c(c2)c(C(C)=O)nn3CC(=O)N2C[C@H](F)C[C@H]2C(=O)Nc2cccc(Br)n2)cn1 ; "and" ; *c1ncc(-c2ccc3c(c2)c(C(C)=O)nn3CC(=O)N2[C@H](C(=O)Nc3nc(Br)ccc3C)C[C@@]3(C)C[C@@H]23)cn1]

### Factor H

In some embodiments, the Target Extracellular Protein is human complement factor H (UniProtKB-P08603 (CFAH_HUMAN)). Complement factor H is a glycoprotein that plays an essential role in maintaining a well-balanced immune response by modulating complement activation. Acts as a soluble inhibitor of complement, where its binding to self-markers such as glycan structures prevents complement activation and amplification on cell surfaces. Complement factor H accelerates the decay of the complement alternative pathway (AP) C3 convertase C3bBb, thus preventing local formation of more C3b, the central player of the complement amplification loop. As a cofactor of the serine protease factor I, CFH also regulates proteolytic degradation of already-deposited C3b. In addition, it mediates several cellular responses through interaction with specific receptors. For example, CFH interacts with CR³/ITGAM receptor and thereby mediates the adhesion of human neutrophils to different pathogens. In turn, these pathogens are phagocytosed and destroyed.

The Protein Data Bank website provides the crystal structure of highly similar mutants of complement factor H searchable by 3KXV and 3KZJ (Bhattacharjee, A., et al., Mol Immunol 2010, 47, 1686-1691); as well as the crystal structure of wild type complement factor H bound to various compounds searchable by 2UWN (Prosser, B. E., et al., J Exp Med 2007, 204, 2277); 5WTB (Zhang, Y., et al., Biochem J 2017, 474, 1619-1631); 5032 and 5035 (Xue, X., et al., Nat Struct Mol Biol 2017, 24, 643-651); 4ONT (Blaum, B. S., et al., Nat Chem Biol 2015, 11, 77-82); and 4ZH₁ (Blaum, B. S., et al., Glycobiology 2016, 26, 532-539).

Representative complement factor H Targeting Ligands are provided in FIG. 7. Additional complement factor H Targeting Ligands are provided in, for example, J Immunol 182:6394-6400 (2009), PLOS Pathogens 4: e1000250 (2008), PLOS Pathogens 6: e1001027 (2010), U.S. Pat. No. 10,865,238B¹, U.S. Pat. No. 8,962,795B², U.S. patent application No. 20160317573A1, and U.S. patent application No. 20190315842A1, each of which is incorporated by reference herein.

### Complement Component 5 (C5)

In some embodiments, the Target Extracellular Protein is human complement component (C5) (UniProtKB-P01031 (CO5_HUMAN)). Activation of C5 by a C5 convertase initiates the spontaneous assembly of the late complement components, C5-C9, into the membrane attack complex. C5b has a transient binding site for C6. The C5b-C6 complex is the foundation upon which the lytic complex is assembled.

The Protein Data Bank website provides the crystal structure of Complement Component searchable by 3CU7 (Fredslund, F., Nat Immunol 2008, 9, 753-760); as well as the crystal structure of Complement Component 5 bound to various compound searchable by 515K (Schatz-Jakobsen, J. A., et al, J Immunol 2016, 197, 337-344); 3PVM and 3PRX (Laursen, N. S., et al., EMBO J 2011, 30, 606-616); and 3KLS (Laursen, N. S., et al., Proc Natl Acad Sci 2010, 107, 3681-3686).

Representative Complement Component 5 Targeting Ligands are provided in the figures. Additional Complement Component 5 Targeting Ligands are provided in, for example, J Immunol 197:337-344 (2016), Ther Adv Hematol 10:1-11 (2019), BioDrugs 34:149-158 (2020), Blood 135:884-885 (2020), U.S. patent application No. 20170342139A1, and U.S. patent application Ser. No. 20/200,095307A1, each of which is incorporated by reference herein.

In certain embodiments the Extracellular Targeting Ligand is selected from:

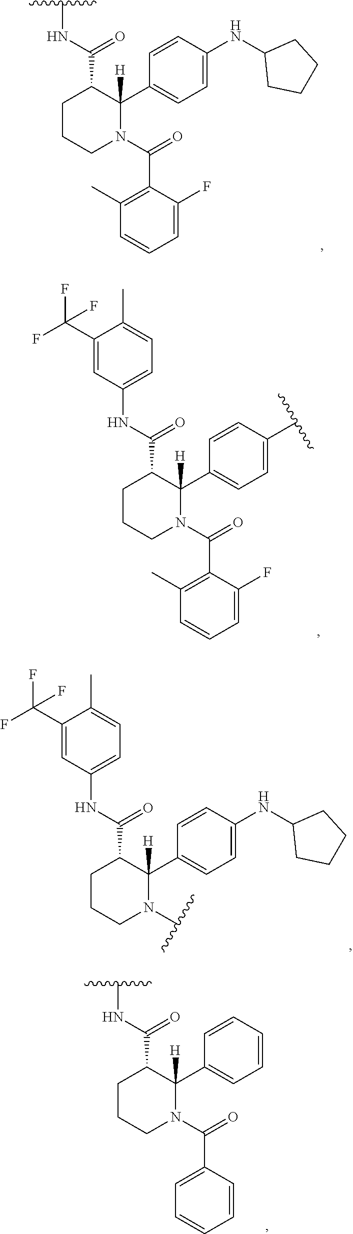

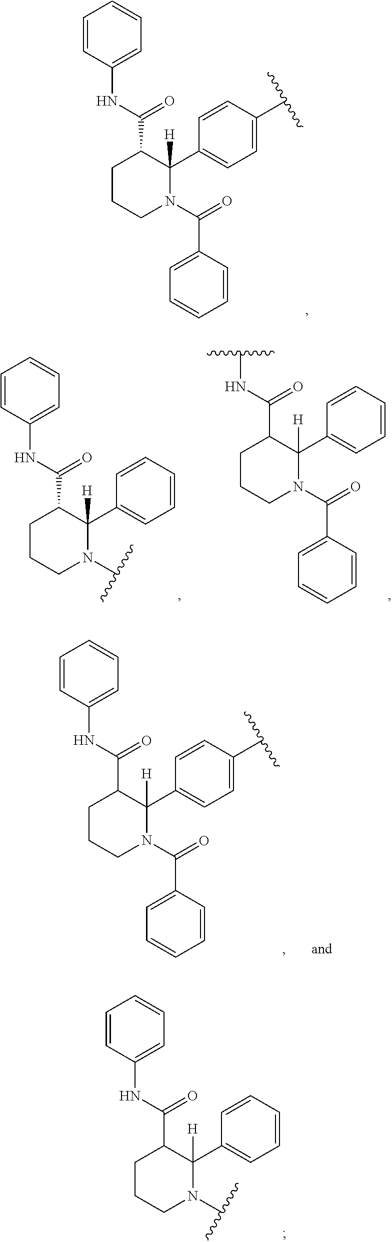 [SMILES: *c1ccc([C@H]2[C@@H](C(=O)Nc3ccccc3)CCCN2C(=O)c2ccccc2)cc1 ; *N1CCC[C@H](C(=O)Nc2ccccc2)[C@@H]1c1ccccc1 ; *NC(=O)C1CCCN(C(=O)c2ccccc2)C1c1ccccc1 ; *c1ccc(C2C(C(=O)Nc3ccccc3)CCCN2C(=O)c2ccccc2)cc1 ; *N1CCCC(C(=O)Nc2ccccc2)C1c1ccccc1]

each of which is optionally substituted with 1, 2, 3, or 4 substituents independently selected from R²¹.

In certain embodiments the complement C5 Targeting Ligand is selected from a ligand described in Jendza, K. et al. A Small-Molecule Inhibitor of C5 Complement Protein. Nat Chem Biol 2019, 15 (7), 666-668; or Zhang, M.; Yang, X.-Y.; Tang, W.; Groeneveld, T. W. L.; He, P.-L.; Zhu, F.-H.; Li, J.; Lu, W.; Blom, A. M.; Zuo, J.-P.; Nan, F.-J. Discovery and Structural Modification of 1-Phenyl-3-(1-Phenylethyl) Urea Derivatives as Inhibitors of Complement. ACS Med. Chem. Lett. 2012, 3 (4), 317-321.

In certain embodiments the C5 Targeting Ligand is selected from:

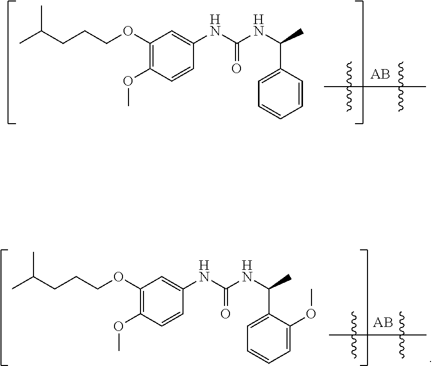

In certain embodiments the C5 Targeting Ligand is selected from:

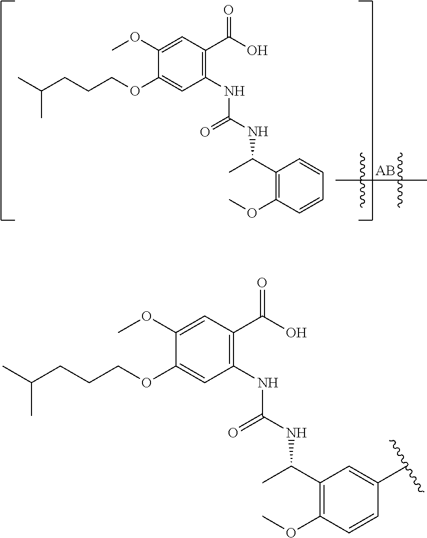

### Complement C1s

In certain embodiments the extracellular targeting ligand is a C1s Targeting Ligand.

In certain embodiments the complement C1s Targeting Ligand is selected from a ligand described in WO2020/198062 or U.S. Pat. No. 6,683,055.

In certain embodiments the compound of the present invention is selected from the following compounds or a bi- or tri-dentate version thereof:

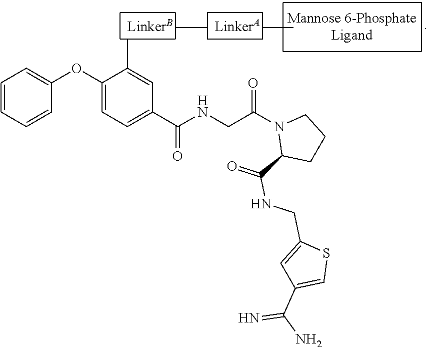 [SMILES: Cc1cc(C(=O)NCC(=O)N2CCC[C@H]2C(=O)NCc2cc(C(=N)N)cs2)ccc1Oc1ccccc1]

### MASP

In certain embodiments the extracellular targeting ligand is a MASP Targeting Ligand.

In certain embodiments the MASP Targeting Ligand is selected from a ligand described in Héja, D. et al. Monospecific Inhibitors Show That Both Mannan-Binding Lectin-Associated Serine Protease-1 (MASP-1) and -2 Are Essential for Lectin Pathway Activation and Reveal Structural Plasticity of MASP-2. Journal of Biological Chemistry 2012, 287 (24), 20290-20300; Dobó, J.; Kocsis, A.; Gál, P. Be on Target: Strategies of Targeting Alternative and Lectin Pathway Components in Complement-Mediated Diseases. Front. Immunol. 2018, 9, 1851; or WO 2014/144542.

In certain embodiments the MSAP-1 Targeting Ligand is SGMI-1 peptide, linked through the N- or C-terminus.

In certain embodiments the MSAP-1 Targeting Ligand is SGMI-2 peptide, linked through the N- or C-terminus.

In certain embodiments the MSAP-1 Targeting Ligand is TFMI-3 peptide, linked through the N- or C-terminus.

### Factor XIa

In certain embodiments the extracellular targeting ligand is a factor XIa Targeting Ligand. In certain embodiments the factor XIa Targeting Ligand is selected from a ligand described in: Lorthiois, E. et al. Structure-Based Design and Preclinical Characterization of Selective and Orally Bioavailable Factor XIa Inhibitors: Demonstrating the Power of an Integrated S1 Protease Family Approach. J. Med. Chem. 2020, 63 (15), 8088-8113.

In certain embodiments the factor XIa Targeting Ligand is selected from a ligand described in: Quan, M. L. et al. Factor XIa Inhibitors as New Anticoagulants. J. Med. Chem. 2018, 61 (17), 7425-7447.

In certain embodiments the factor XIa Targeting Ligand is selected from a ligand described in: Yang, W. et al. Discovery of a High Affinity, Orally Bioavailable Macrocyclic FXIa Inhibitor with Antithrombotic Activity in Preclinical Species. J. Med. Chem. 2020, 63 (13), 7226-7242.

In certain embodiments the factor XIa Targeting Ligand-Linker is:

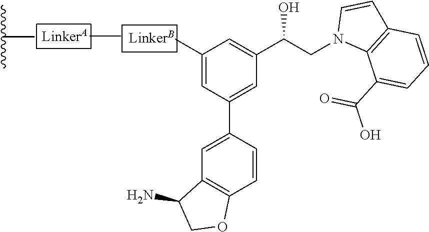

In certain embodiments the compound of the present invention is selected from the following compounds or a bi- or tri-dentate version thereof:

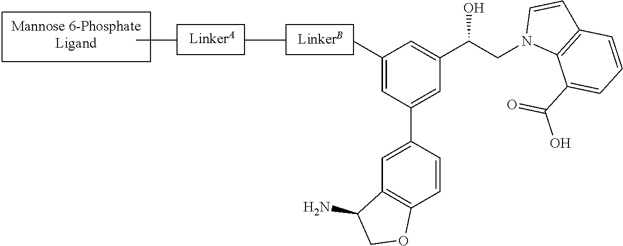 [SMILES: Cc1cc(-c2ccc3c(c2)[C@H](N)CO3)cc([C@H](O)Cn2ccc3cccc(C(=O)O)c32)c1]

In certain embodiments the factor Xia Targeting Ligand is selected where an anchor bond is placed at any suitable location with or without functionalization.

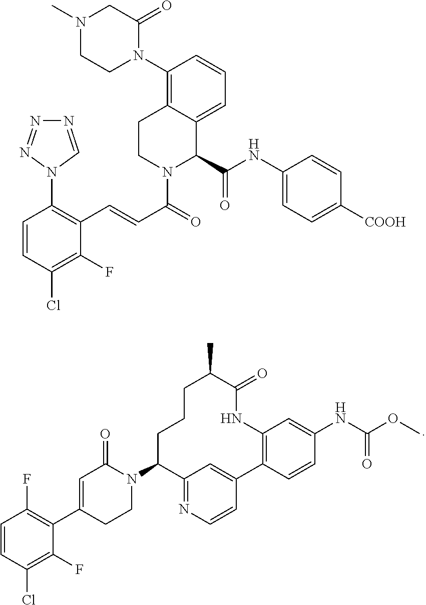 [SMILES: CN1CCN(c2cccc3c2CCN(C(=O)/C=C/c2c(-n4cnnn4)ccc(Cl)c2F)[C@@H]3C(=O)Nc2ccc(C(=O)O)cc2)C(=O)C1 ; COC(=O)Nc1ccc2c(c1)NC(=O)[C@H](C)CCC[C@H](N1CCC(c3c(F)ccc(Cl)c3F)=CC1=O)c1cc-2ccn1]

In certain embodiments the Factor XIa Targeting Ligand is selected from:

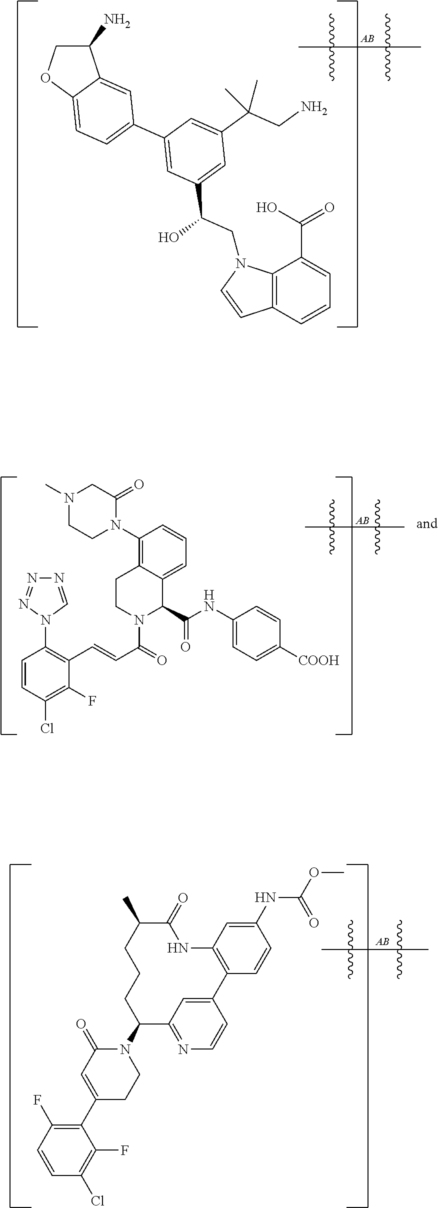

### Immunoglobulin Degradation

Immunoglobulins, for example IgG, can cause, modulate, or amplify diseases in vivo, such as abnormal cellular proliferation such as tumors and cancer, autoimmune disorders, inflammation, and aging-related diseases. For example, immunoglobulins bind to cell surface receptors, often initiating aberrant signaling in multiple diseases such as cancer and inflammation.

The immunoglobulin degraders described herein or their pharmaceutically acceptable salt and/or pharmaceutically acceptable compositions thereof can be used to treat a disorder which is mediated by an immunoglobulin that binds to the Immunoglobulin Targeting Ligand. The described degraders are capable of targeting immunoglobulins that mediate pathological disorders for lysosomal degradation. The selected immunoglobulin may modulate a disorder in a human via a mechanism of action such as modification of a biological pathway, pathogenic signaling, or modulation of a signal cascade or cellular entry. The immunoglobulin is recruited with an Immunoglobulin Targeting Ligand, which is a ligand for the immunoglobulin.

Accordingly, in some embodiments, a method to treat a host with a disorder mediated by an immunoglobulin is provided that includes administering an effective amount of a degrader targeting the immunoglobulin or its pharmaceutically acceptable salt described herein to the host, typically a human, optionally in a pharmaceutically acceptable composition.

The immunoglobulin can be either the normal form of the protein or an aberrant form. For example, the immunoglobulin can be a mutant protein, or a protein, for example, where a partial, or full, gain-of-function or loss-of-function is encoded by nucleotide polymorphisms.

Targeting specific immunoglobulins is accomplished by the present invention through the use of specific Immunoglobulin Targeting Ligands. The target immunoglobulins of the current invention may include, but are not limited to, immunoglobulin G (IgG), immunoglobulin A (IgA), and immunoglobulin E (IgE). These immunoglobulins mediate a range of diseases that can be treated with an effective amount of the disclosed Immunoglobulin Degraders described herein.

### Immunoglobulin A (IgA)

Aberrant expression of immunoglobulin A (IgA) mediates a range of autoimmune and immune-mediated disorders, including IgA nephropathy (also known as Berger's disease), celiac disease, Crohn's disease, Henoch-Schönlein purpura (HSP) (also known as IgA vasculitis), IgA pemphigus, dermatitis herpetiformis, inflammatory bowel disease (IBD), Sjögren's syndrome, ankylosing spondylitis, alcoholic liver cirrhosis, acquired immunodeficiency syndrome, IgA multiple myeloma, α-chain disease, IgA monoclonal gammopathy, monoclonal gammopathy of undetermined significance (MGUS), linear IgA bullous dermatosis, rheumatoid arthritis, ulcerative colitis, and primary glomerulonephritis, among others.

Specific degradation of IgA can be accomplished through the use of an IgA-specific Immunoglobulin Targeting Ligand. In certain embodiments, the Immunoglobulin Targeting Ligand used is an Opt peptide. Variations and derivatives of the IgA-specific Opt peptide suitable for use as IgA-specific Immunoglobulin Targeting Ligands are described in Hatanaka et al. Journal of Biological Chemistry, 287 (51) 43126-43136. In certain embodiments, the IgA-specific Immunoglobulin Targeting Ligand is Opt-1. In certain embodiments, the IgA-specific Immunoglobulin Targeting Ligand is Opt-2. In certain embodiments, the IgA-specific Immunoglobulin Targeting Ligand is Opt-3.

In certain embodiments the immunoglobulin degrading compound is:

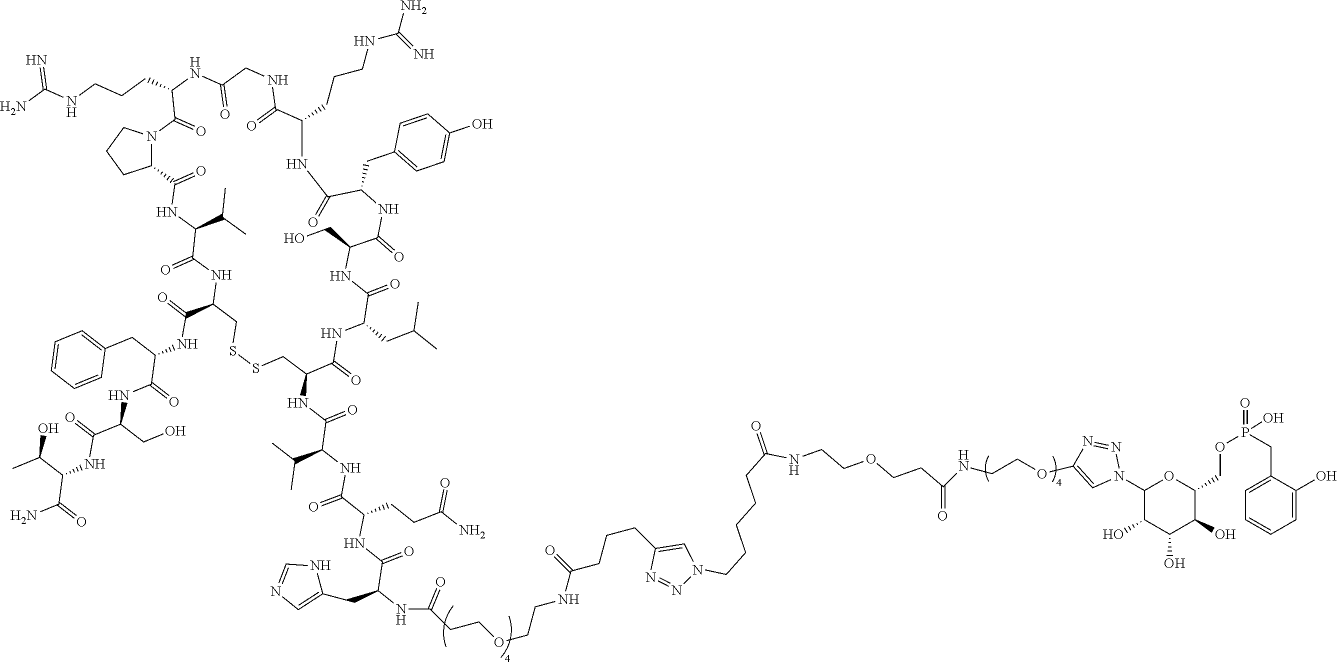

or a pharmaceutically acceptable salt thereof.

In certain embodiments the immunoglobulin degrading compound is:

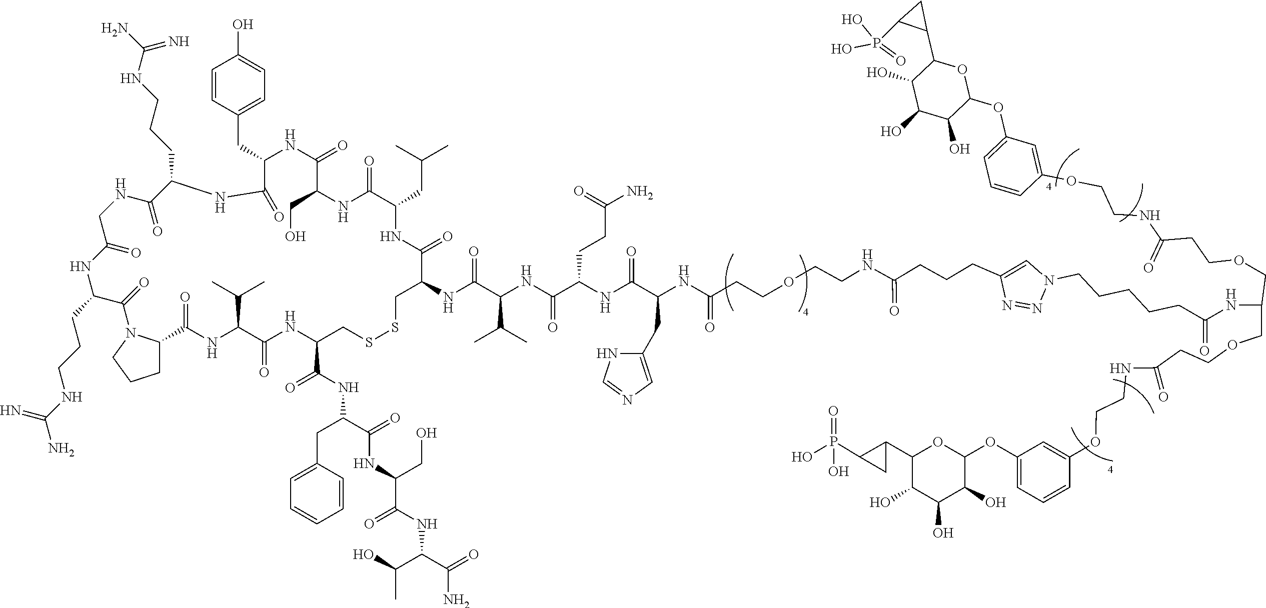

or a pharmaceutically acceptable salt thereof.

In certain embodiments the immunoglobulin degrading compound is:

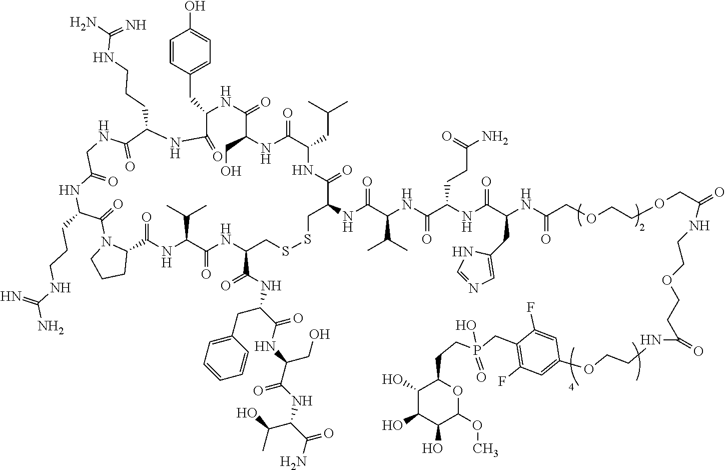

or a pharmaceutically acceptable salt thereof.

In certain embodiments the Immunoglobulin Targeting Ligand is:

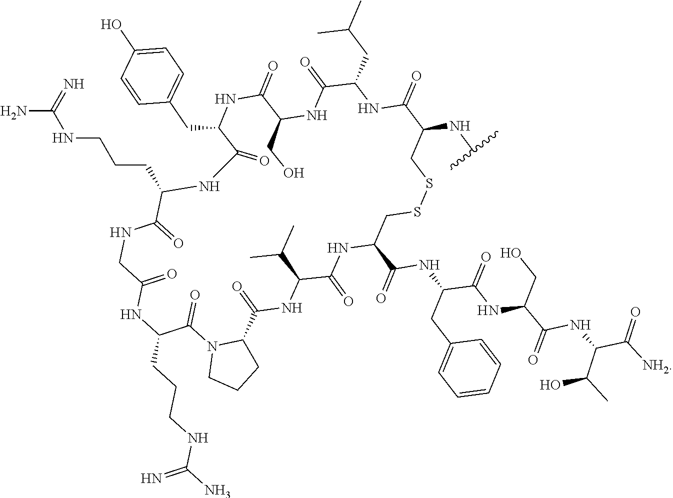 [SMILES: *N[C@H]1CSSC[C@@H](C(=O)N[C@@H](Cc2ccccc2)C(=O)N[C@@H](CO)C(=O)N[C@H](C(N)=O)[C@@H](C)O)NC(=O)[C@H](C(C)C)NC(=O)[C@@H]2CCCN2C(=O)[C@H](CCCNC(=N)N)NC(=O)CNC(=O)[C@H](CCCNC(=N)N)NC(=O)[C@H](Cc2ccc(O)cc2)NC(=O)[C@H](CO)NC(=O)[C@H](CC(C)C)NC1=O]

The Protein Data Bank website provides the crystal structure of IgA, as well as the crystal structure of IgA bound to various compounds searchable by SE8E (Baglin, T. P., et al., J. Thromb. Haemost., 2016, 14:137-142), and 2QTJ (Bonner, A., et al., J. Immunol., 2008, 180:1008-1018). Additionally, Hatanaka T. et al., provides great insight into the specificity and high binding affinity of IgA to OPT-1 peptides (J Biol Chem., 2012, 287 (51), 43126-43136.).

Representative IgA Targeting Ligands are provided in FIG. 1.

Additional representative Targeting Ligands include:

SEQ ID NO: 1
MLKKIE (Jerlstrom et al. Infect. Immun. 1996 Jul.;
64(7): 2787-2793;
SEQ ID NO: 2
VEPNCDIHVMWEWECFERL (Biochemistry 1998, 37, 17754-
177764)
SEQ ID NO: 3
TVFTSWEEYLDWV (J. Bio. Chem. 2014 Jan.; 289(2):
942-955)
SEQ ID NO: 4
*(Ac)-FVPTTX(N-Me)AX(N-Me)AEAPC*
SEQ ID NO: 5
*(Ac)-FVDTTS(N-Me)FX(N-Me)ENSPC*
SEQ ID NO: 6
*(Ac)-FVSTTX(N-Me)AX(N-Me)ADRPC*
SEQ ID NO: 7
*(Ac)-FVDTTS(N-Me)FX(N-Me)ANSPC*
SEQ ID NO: 8
*(Ac)-FVDSTT(N-Me)AX(N-Me)ANHPC*
SEQ ID NO: 9
*(Ac)-FVDTTS(N-Me)FX(N-Me)AESPC*
SEQ ID NO: 10
*(Ac)-FVDTTS(N-Me)F(4CF)F(N-Me)AESPC*
SEQ ID NO: 11
*(Ac)-FVDTTS(N-Me)AX(N-Me)AKSPC*
SEQ ID NO: 12
*(Ac)-FVSTTX(N-Me)AX(N-Me)ADSPC*
SEQ ID NO: 13
*(Ac)-FVSTTS(N-Me)FX(N-Me)ADRPC*
SEQ ID NO: 14
*(Ac)-FVDTTX(N-Me)AX(N-Me)AESPC*
SEQ ID NO: 15
*(Ac)-FVSTT(4CF₃)F(N-Me)A(4CF₃)F(N-Me)AERPC*
SEQ ID NO: 16
*(Ac)-FVSTTS(N-Me)FX(N-Me)AESPC*
SEQ ID NO: 17
*(Ac)-FVSTTX(N-Me)FX(N-Me)AESPC*
SEQ ID NO: 18
*(Ac)-FVSTTX(N-Me)A(3,4diCI)F(N-Me)ADRPC*
SEQ ID NO: 19
*(Ac)-FVSTTX(N-Me)A(3FI)F(N-Me)ADRPC*
SEQ ID NO: 20
*(Ac)-FVDTTA(N-Me)FX(N-Me)AEAPC*
SEQ ID NO: 21
*(Ac)-FVDTTF(N-Me)AX(N-Me)AESPC*
SEQ ID NO: 22
*(Ac)-FVDTTA(N-Me)FX(N-Me)AQAPC*
SEQ ID NO: 23
*(Ac)-FVPTTX(N-Me)AX(N-Me)ADRPC*
SEQ ID NO: 24
*(Ac)-FVSTTX(N-Me)AX(N-Me)APSPC*
SEQ ID NO: 25
*(Ac)-FVPTTA(N-Me)FX(N-Me)AEAPC*
SEQ ID NO: 26
*(Ac)-FVATTF(N-Me)AX(N-Me)AKAPC*
SEQ ID NO: 27
*(Ac)-FVNTTF(N-Me)AX(N-Me)AKAPC*
SEQ ID NO: 28
*(Ac)-FVSTTF(N-Me)AX(N-Me)AEAPC*
SEQ ID NO: 29
*(Ac)-FVSTTX(N-Me)AX(N-Me)AESPC*
SEQ ID NO: 30
*(Ac)-FVDTTX(N-Me)A(3,4diCI)F(N-Me)AESPC*
SEQ ID NO: 31
*(Ac)-FENTTF(N-Me)AX(N-Me)AASPC*
SEQ ID NO: 32
*(Ac)-FVPTTF(N-Me)A(4CI)F(N-Me)APAPC*
SEQ ID NO: 33
*(Ac)-FVPTTF(N-Me)A(4CI)F(N-Me)ADAPC*
SEQ ID NO: 34
*(Ac)-FV-Hse-TTF(N-Me)A(4CI)F(N-Me)ADAPC*
SEQ ID NO: 35
*(Ac)-FVATTF(N-Me)A(4CI)F(N-Me)ANAPC*
SEQ ID NO: 36
*(Ac)-FVPTTV(N-Me)A(4CI)F(N-Me)AEAPC*
SEQ ID NO: 37
*(Ac)-FVNTTF(N-Me)AX(N-Me)A(N-Me)EAPC*
SEQ ID NO: 38
*(Ac)-FVPTTF(N-Me)AX(N-Me)AEAPC*
SEQ ID NO: 39
*(Ac)-FVPTTX(N-Me)AX(N-Me)AAAPC*
SEQ ID NO: 40
Opt-1 - HMVCLAYRGRPVCFAL (Hatanaka et al. J. Biol.
Chem. Vol. 287, No. 51, pp. 43126-43136, Dec. 14,
2012)
SEQ ID NO: 41
Opt-2 - HMVCLSYRGRPVCFSL (Hatanaka et al. J. Biol.
Chem. Vol. 287, No. 51, pp. 43126-43136, Dec. 14,
2012)
SEQ ID NO: 42
Opt-3 - HQVCLSYRGRPVCFST (Hatanaka et al. J. Biol.
Chem. Vol. 287, No. 51, pp. 43126-43136, Dec. 14,
2012)
SEQ ID NO: 43
QMRCLSYKGRRVCLWL (U.S. Pat. No. 9593147)
SEQ ID NO: 44
KRLCLQYKGSKVCFRL (U.S. Pat. No. 9593147)
SEQ ID NO: 45
RMRCLTYRGRRVCLEL (U.S. Pat. No. 9593147)
SEQ ID NO: 46
SMRCLQYRGSRVCLTL (U.S. Pat. No. 9593147)
SEQ ID NO: 47
HLRCLRYKGTRVCFSL (U.S. Pat. No. 9593147)
SEQ ID NO: 48
HVRCLSYKGREVCVQL (U.S. Pat. No. 9593147)
SEQ ID NO: 49
PRMCLFIYKGRRVCIPY (U.S. Pat. No. 9593147)
SEQ ID NO: 50
HMRCLHYKGRRVCFLL (U.S. Pat. No. 9593147)
SEQ ID NO: 51
HKRCLHYRGRMVCFLI (U.S. Pat. No. 9593147)
SEQ ID NO: 52
QKRCLKYKGSRVCFFL (U.S. Pat. No. 9593147)
SEQ ID NO: 53
HVRCLRYRGKNVCFLL (U.S. Pat. No. 9593147)
SEQ ID NO: 54
SDVCLRYRGRPVCFQV (U.S. Pat. No. 9593147)
SEQ ID NO: 55
RDVCLRYRGRPVCFQV (U.S. Pat. No. 9593147)
SEQ ID NO: 56
HDVCLRYRGRPVCFQV (U.S. Pat. No. 9593147)
SEQ ID NO: 57
SMVCLRYRGRPVCFQV (U.S. Pat. No. 9593147)
SEQ ID NO: 58
SAVCLRYRGRPVCFQV (U.S. Pat. No. 9593147)
SEQ ID NO: 59
SDVCLNYRGRPVCFQV (U.S. Pat. No. 9593147)
SEQ ID NO: 60
SDVCLHYRGRPVCFQV (U.S. Pat. No. 9593147)
SEQ ID NO: 61
SDVCLAYRGRPVCFQV (U.S. Pat. No. 9593147)
SEQ ID NO: 62
SDVCLRYRGRPVCFAV (U.S. Pat. No. 9593147)
SEQ ID NO: 63
SDVCLRYRGRPVCFQL (U.S. Pat. No. 9593147)
SEQ ID NO: 64
SDVCLRYRGRPVCFQA (U.S. Pat. No. 9593147)
SEQ ID NO: 65
HMVCLSYRGRPVCF (US Pub. No. 20150044701)
SEQ ID NO: 66
HMVCLSYRGRPVCFS (US Pub. No. 20150044701)
SEQ ID NO: 67
HQVCLSYRGQPVCFSL (US Pub. No. 20150044701)
SEQ ID NO: 68
HQVCLSYRGRPTCFSL (US Pub. No. 20150044701)
SEQ ID NO: 69
HQVCLSYRGRPVCYSL (US Pub. No. 20150044701)
SEQ ID NO: 70
HQVCLSYRGQPVCFST (US Pub. No. 20150044701)
SEQ ID NO: 71
HQVCLSYRGRPTCFST (US Pub. No. 20150044701)
SEQ ID NO: 72
HQVCLSYRGQPTCFST (US Pub. No. 20150044701)

In certain embodiments the IgA Targeting Ligand is

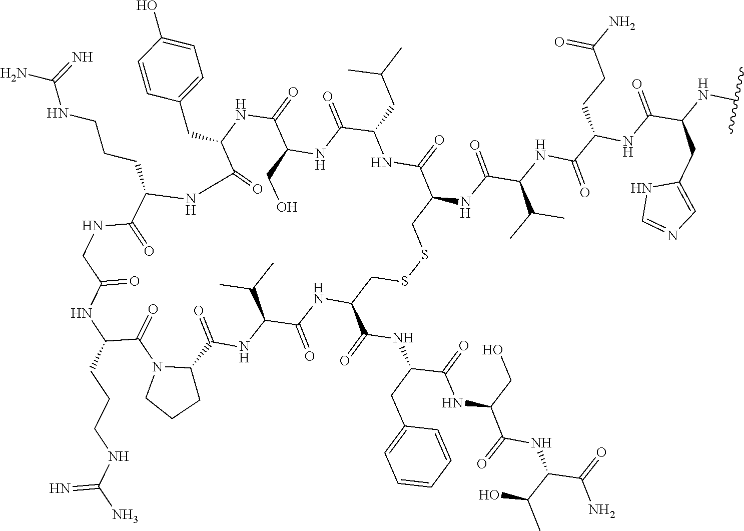 [SMILES: *N[C@@H](Cc1cnc[nH]1)C(=O)N[C@@H](CCC(N)=O)C(=O)N[C@H](C(=O)N[C@H]1CSSC[C@@H](C(=O)N[C@@H](Cc2ccccc2)C(=O)N[C@@H](CO)C(=O)N[C@H](C(N)=O)[C@@H](C)O)NC(=O)[C@H](C(C)C)NC(=O)[C@@H]2CCCN2C(=O)[C@H](CCCNC(=N)N)NC(=O)CNC(=O)[C@H](CCCNC(=N)N)NC(=O)[C@H](Cc2ccc(O)cc2)NC(=O)[C@H](CO)NC(=O)[C@H](CC(C)C)NC1=O)C(C)C]

### Immunoglobulin G (IgG)

Immunoglobulin G (IgG) mediates a range of autoimmune, infectious and metabolic diseases, including systemic fibroinflammatory disease. In addition, overexpression of IgG4 is associated with IgG4-related diseases, which generally include multiple organs, and disorders include type 1 autoimmune pancreatitis, interstitial nephritis, Riedel's thyroiditis, storiform fibrosis, Mikulicz's disease, Küttner's tumor, inflammatory pseudotumors (in various sites of the body), mediastinal fibrosis, retroperitoneal fibrosis (Ormond's disease), aortitis and periaortitis, proximal biliary strictures, idiopathic hypocomplementemic tubulointerstitial nephritis, multifocal fibrosclerosis, pachymeningitis, pancreatic enlargement, tumefactive lesions, pericarditis, rheumatoid arthritis (RA), inflammatory bowel disease, multiple sclerosis, myasthenia gravis, ankylosing spondylitis, primary Sjögren's syndrome, psoriatic arthritis, systemic lupus erythematosus (SLE), sclerosing cholangitis, IgG monoclonal gammopathy, monoclonal gammopathy of undetermined significance (MGUS), melanoma, bullous pemphigoid, Goodpasture disease, encephalitis, thrombotic thrombocytopeniaurpura, chronic inflammatory polyneuropathy, limbic encephalitis, neuromyotonia, Morvan syndrome, pemphigus foliaceus, pemphigus vulgaris, REM and non-REM parasomnia, and membranous nephropathy, multiple sclerosis, hyperthyroid Grave's disease, epidermolysis bullosa acquisita, pemphigoid gestationis, anti-p200 pemphigoid, and paraneoplastic pemphigus, among others.

Specific degradation of IgG can be accomplished through the use of an IgG-specific Immunoglobulin Targeting Ligand. In certain embodiments, the Immunoglobulin Targeting Ligand binds to the Fc region of IgG. In certain embodiments the IgG-specific Immunoglobulin Targeting Ligand is an Fc-binding peptide. In certain embodiments, the IgG-specific Immunoglobulin Targeting Ligand is Fc-BP2. In certain embodiments, the IgG-specific Immunoglobulin Targeting Ligand is Fc-III.

In certain embodiments the Immunoglobulin Targeting Ligand is:

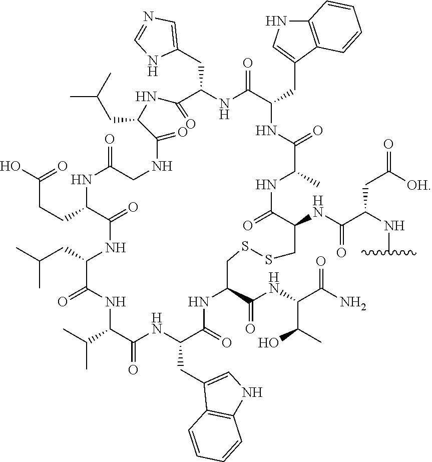 [SMILES: *N[C@@H](CC(=O)O)C(=O)N[C@H]1CSSC[C@@H](C(=O)N[C@H](C(N)=O)[C@@H](C)O)NC(=O)[C@H](Cc2c[nH]c3ccccc23)NC(=O)[C@H](C(C)C)NC(=O)[C@H](CC(C)C)NC(=O)[C@H](CCC(=O)O)NC(=O)CNC(=O)[C@H](CC(C)C)NC(=O)[C@H](Cc2cnc[nH]2)NC(=O)[C@H](Cc2c[nH]c3ccccc23)NC(=O)[C@H](C)NC1=O]

In certain embodiments the Immunoglobulin Targeting Ligand is:

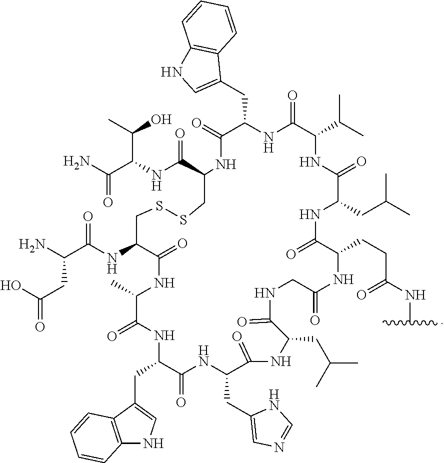 [SMILES: *NC(=O)CC[C@@H]1NC(=O)CNC(=O)[C@H](CC(C)C)NC(=O)[C@H](Cc2cnc[nH]2)NC(=O)[C@H](Cc2c[nH]c3ccccc23)NC(=O)[C@H](C)NC(=O)[C@@H](NC(=O)[C@@H](N)CC(=O)O)CSSC[C@@H](C(=O)N[C@H](C(N)=O)[C@@H](C)O)NC(=O)[C@H](Cc2c[nH]c3ccccc23)NC(=O)[C@H](C(C)C)NC(=O)[C@H](CC(C)C)NC1=O]

In certain embodiments the Immunoglobulin Targeting Ligand is:

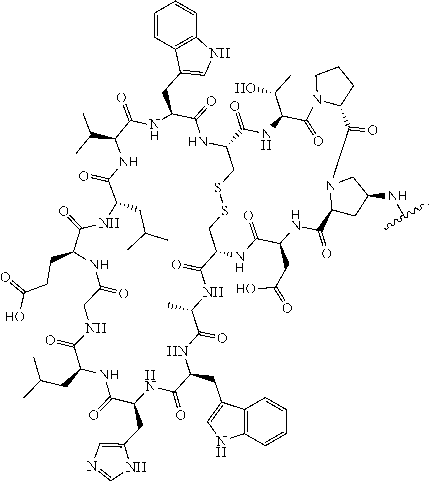 [SMILES: *N[C@H]1C[C@H]2C(=O)N[C@@H](CC(=O)O)C(=O)N[C@H]3CSSC[C@H](NC(=O)[C@H](Cc4c[nH]c5ccccc45)NC(=O)[C@H](C(C)C)NC(=O)[C@H](CC(C)C)NC(=O)[C@H](CCC(=O)O)NC(=O)CNC(=O)[C@H](CC(C)C)NC(=O)[C@H](Cc4cnc[nH]4)NC(=O)[C@H](Cc4c[nH]c5ccccc45)NC(=O)[C@H](C)NC3=O)C(=O)N[C@@H]([C@@H](C)O)C(=O)N3CCC[C@@H]3C(=O)N2C1]

The Protein Data Bank website provides the crystal structure of IgG searchable by 1H3X (Krapp, S., et al., J. Mol. Biol., 2003, 325:979); and 5V43 (Lee, C. H., et al., Nat. Immunol., 2017, 18:889-898); as well as the crystal structure of IgG bound to various compounds searchable by 5YC5 (Kiyoshi M., et al., Sci. Rep., 2018, 8:3955-3955); 5XJE (Sakae Y., et al., Sci. Rep.,2017, 7:13780-13780); 5GSQ (Chen, C. L., et al., ACS Chem. Biol., 2017, 12:1335-1345); and 1HZH (Saphire E. O., et al., Science, 2001, 293:1155-1159). Additionally, Kiyoshi, M., et al., provides insight into the structural basis for binding of human IgG1 to its high-affinity human receptor 10 FcγRI. (Kiyosi M., et al., Nat Commun., 2015, 6, 6866).

Representative IgG Targeting Ligands are provided in FIG. 1.

Additional representative IgG Targeting Ligands include:

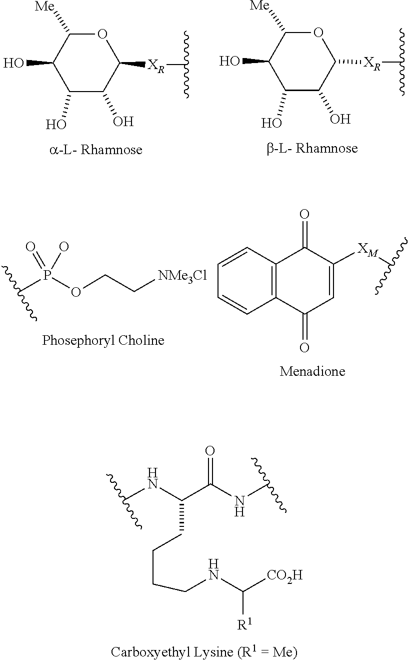

wherein X_{R} is O, S, NH, or N—C₁-C₃ alkyl; and
X_{M} is O, S, NH, or N—C₁-C₃ alkyl.

In other embodiments the IgG Targeting Ligand is selected from:

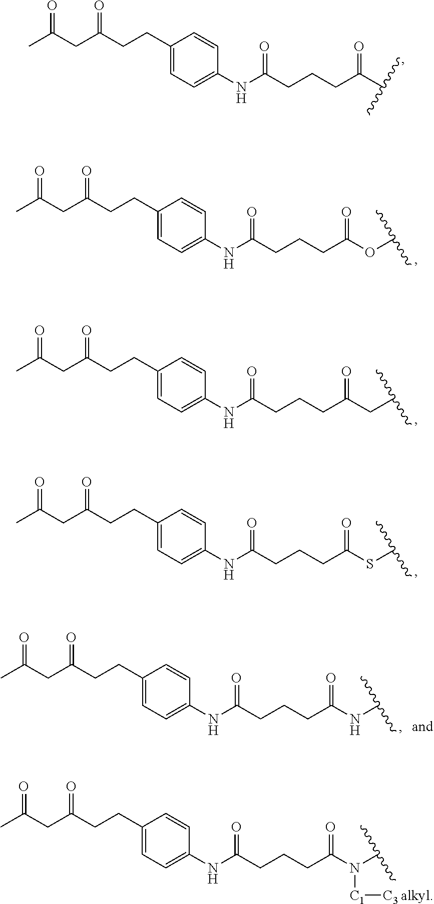

In some embodiments, the IgG Targeting Ligand is a group according to the chemical structure:

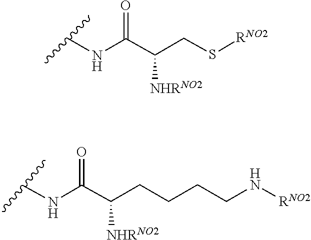

wherein R^{N02} is a dinitrophenyl group optionally linked through CH₂, S(O), S(O)₂, —S(O)₂O, —OS(O)₂, or OS(O)₂O.

In certain embodiments the IgG Targeting Ligand is selected from:

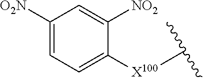

wherein X¹⁰⁰ is selected from O, CH₂, NH, N—C₁-C₃ alkyl, NC(O) C₁-C₃ alkyl, S(O), S(O)₂, —S(O)₂O, —OS(O)₂, or OS(O)₂O.

In some embodiments, the IgG Targeting Ligand is a 3-indoleacetic acid group according to the chemical structure:

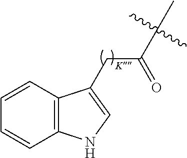

where k″″ is 1˜4 (preferably 2-3, most often 3) or a

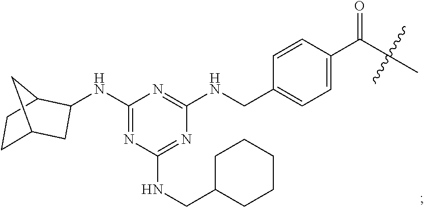 [SMILES: *C(=O)c1ccc(CNc2nc(NCC3CCCCC3)nc(NC3CC4CCC3C4)n2)cc1]

group.

In some embodiments, the IgG Targeting Ligand is a peptide. Nonlimiting examples of IgG Targeting Ligand peptides include:

SEQ ID NO: 73
PAM(RTY)₄K₂KG
(Fassina, et al, J. Mol. Recognit. 1996,
9, 564-569)

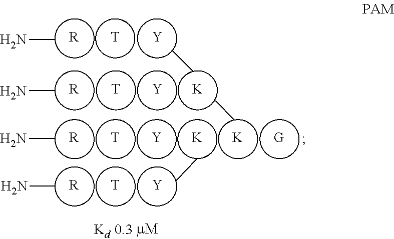

D-PAM, wherein the amino acids of the PAM sequence are all D-amino acids (Verdoliva, et al, J. Immunol. Methods, 2002, 271, 77-88) SEQ ID NO: 74 (RTY)₄K₂KG D-PAM-Φ, wherein the amino acids of the PAM sequence are all D-amino acids with further modifications wherein the four N-terminal arginines are acetylated with phenylactic acid (Dinon, et al J. Mol. Recognit. 2011, 24, 1087-1094) SEQ ID NO: 75 (RTY)₄K₂KG

SEQ ID NO: 76
TWKTSRISIF
(Krook, et al, .J. Immunol. Methods 1998,
221, 151-157)
SEQ ID NO: 77
FGRLVSSIRY
(Krook, et al, J. Immunol. Methods 1998,
221, 151-157)
Fc-III
SEQ ID NO: 78
(DCAWHLGELVWCT-NH2)
(DeLano et al, Science 2000, 287, 1279-1283)

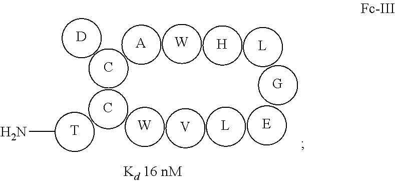

SEQ ID NO: 79
FCBP-Ser DSAWHLGELWST
(see WO2014010813)
SEQ ID NO: 80
DCHKRSFWADNCT
(see WO2014010813)
SEQ ID NO: 81
DCRTQFRPNQTCT
(see WO2014010813)
SEQ ID NO: 82
DCQLCDFWRTRCT
(see WO2014010813)
SEQ ID NO: 83
DCFEDFNEQRTCT
(see WO2014010813)
SEQ ID NO: 84
DCLAKFLKGKDCT
(see WO2014010813)
SEQ ID NO: 85
DCWHRRTHKTFCT
(see WO2014010813)
SEQ ID NO: 86
DCRTIQTRSCT
(see WO2014010813)
SEQ ID NO: 87
DCIKLAQLHSVCT
(see WO2014010813)
SEQ ID NO: 88
DCWRHRNATEWCT
(see WO2014010813)
SEQ ID NO: 89
DCQNWIKDVHKCT
(see WO2014010813)
SEQ ID NO: 90
DCAWHLGELVWCT
(see WO2014010813)
SEQ ID NO: 91
DCAFHLGEL VWCT
(see WO2014010813)
SEQ ID NO: 92
DCAYHLGELVWCT
(see WO2014010813)
FcBP-1
SEQ ID NO: 93
PAWHLGELVWP
(Kang, et al, J. Chromatogr. A 2016, 1466,
105-1 12)

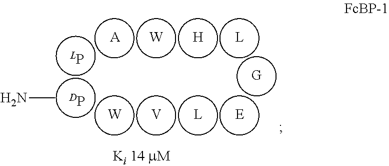

FcBP-2
SEQ ID NO: 94
PDCAWHLGELVWCTP
(Dias, et al, J. Am. Chem. Soc. 2006,
128, 2726-2732)

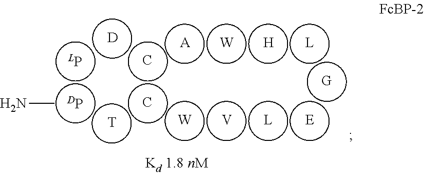

Fc-111-4c
SEQ ID NO: 95
CDCAWHLGELVWCTC
(Gong, et al, Bioconjug. Chem. 2016, 27,
1569-1573)

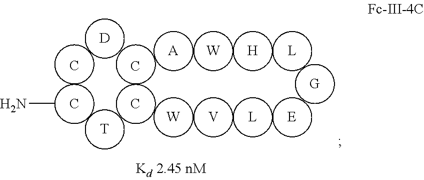

SEQ ID NO: 96
EPIHRSTLTALL
(Ehrlich, et al, J. Biochem. Biophys.
Method 2001, 49, 443-454)
SEQ ID NO: 97
APAR
(Camperi, et al, Biotechnol. Lett. 2003,
25, 1545-1548)
SEQ ID NO: 98
FcRM
(CFHH)₂KG
(Fc Receptor Mimetic, Verdoliva, et al.,
ChemBioChem 2005, 6, 1242-1253)

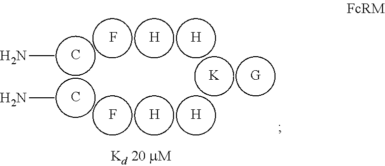

SEQ ID NO: 99
HWRGWV
(Yang, et al., J Peptide Res. 2006, 66, 1 1 0-137)
SEQ ID NO: 100
HYFKFD
(Yang, et al, J. Chromatogr. A 2009, 1216, 910-918)
SEQ ID NO: 101
HFRRHL
(Menegatti, et al, J. Chromatogr. A 2016, 1445, 93-104)
SEQ ID NO: 102
HWCitGWV
(Menegatti, et al, J. Chromatogr. A 2016, 1445, 93-104)
SEQ ID NO: 103
HWmetCitGWmetV
(US10, 266, 566)
SEQ ID NO: 104
D₂AAG
(Small Synthetic peptide ligand, Lund, et al, J. Chromatogr. A
2012, 1225, 158-167)
SEQ ID NO: 105
DAAG
(Small Synthetic peptide ligand, Lund, et al, J. Chromatogr. A
2012, 1225, 158-167);
SEQ ID NO: 106
cyclo[(Nα-Ac) S(A)-RWHYFK-Lact-E]
(Menegatti, et al, Anal. Chem.2013, 85, 9229-9237);
SEQ ID NO: 107
cyclo[(Nα-Ac)-Dap(A)-RWHYFK-Lact-E]
(Menegatti, et al, Anal. Chem. 2013, 85, 9229-9237);
SEQ ID NO: 108
cyclo[Link M-WFRHYK]
(Menegatti, et al, Biotechnol. Bioeng. 2013, 110, 857-870);
SEQ ID NO: 109
NKFRGKYK
(Sugita, et al, Biochem. Eng. J. 2013, 79, 33-40);
SEQ ID NO: 110
NARKFYKG
(Sugita, et al, Biochem. Eng. J. 2013, 79, 33-40);
SEQ ID NO: 111
FYWHCLDE
(Zhao, et al, Biochem. Eng. J. 2014, 88, 1-11);
SEQ ID NO: 112
FYCHWALE
(Zhao, et al, J Chromatogr. A 2014, 1355, 107-114);
SEQ ID NO: 113
FYCHTIDE
(Zhao, et al., Z Chromatogr. A 2014, 1359, 100-111);
Dual 1/3
SEQ ID NO: 114
(FYWHCLDE-FYCHTIDE)
(Zhao, et al, J. Chromatogr. A 2014, 1369, 64-72);
SEQ ID NO: 115
RRGW
(Tsai, et al, Anal. Chem. 2014, 86, 293 1-2938);
SEQ ID NO: 116
KHRFNKD
(Yoo and Choi, BioChip J. 2015, 10, 88-94);
SEQ ID NO: 117
CPSTHWK
(Sun et al. Polymers 2018, 10, 778);
SEQ ID NO: 118
NVQYFAV
(Sun et al. Polymers 2018, 10, 778);
SEQ ID NO: 119
ASHTQKS
(Sun et al. Polymers 2018, 10, 778);
SEQ ID NO: 120
QPQMSHM
(Sun et al. Polymers 2018, 10, 778);
SEQ ID NO: 121
TNIESLK
(Sun et al. Polymers 2018, 10, 778);
SEQ ID NO: 122
NCHKCWN
(Sun et al. Polymers 2018, 10, 778);
SEQ ID NO: 123
SHLSKNF
(Sun et al. Polymers 2018, 10, 778).

In some embodiments the IgG Targeting Ligand is specific for IgG4.

In some embodiments the IgG4 specific Targeting Ligand is described in Gunnarsson et al. Biomolecular Engineering 2006, 23, 111-117.

In some embodiments the IgG4 specific targeting ligand is selected from

SEQ ID NO: 124
FDLLEHFY
and
SEQ ID NO: 125
DLLHHFDYF.

Additional IgG Targeting Ligands include

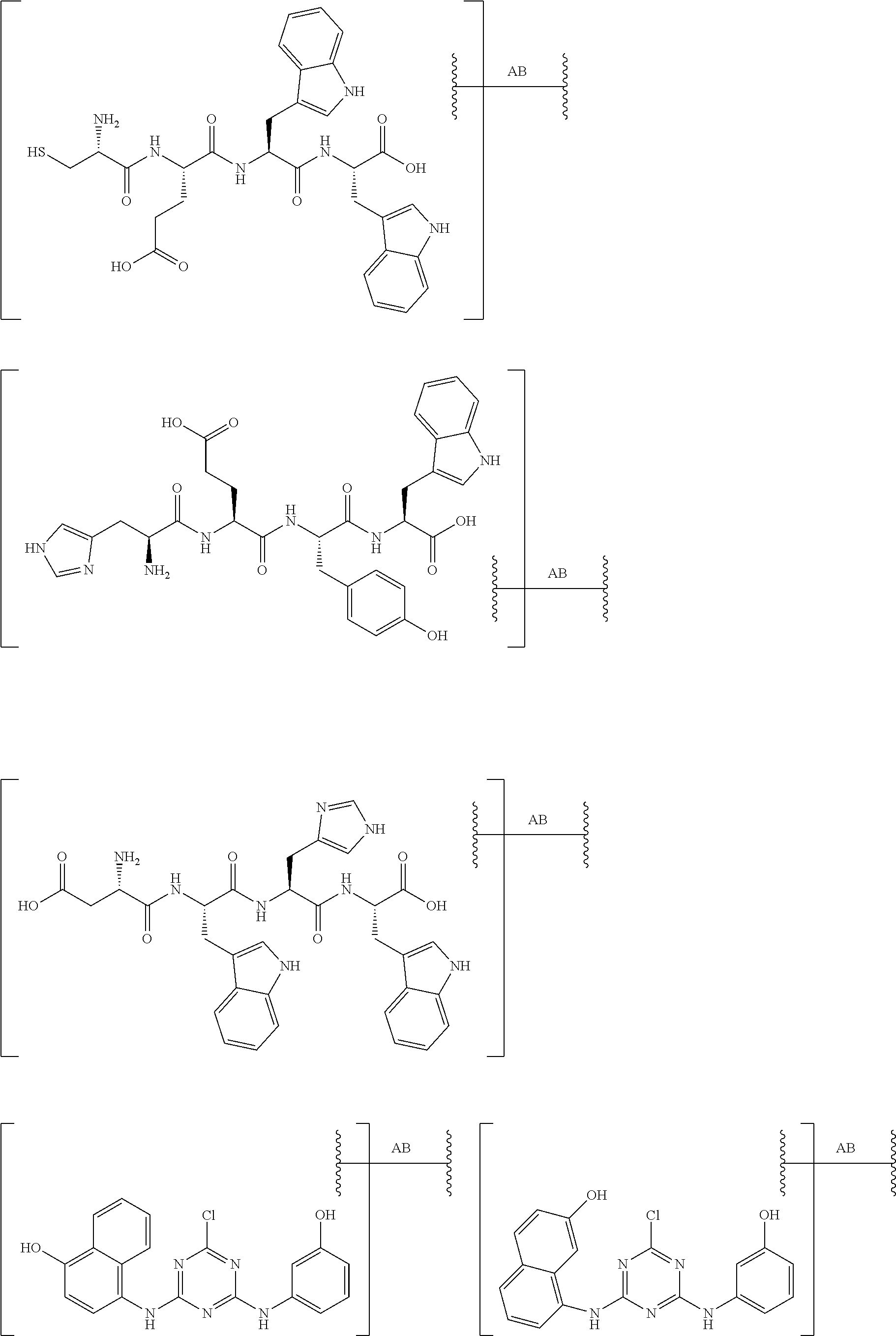

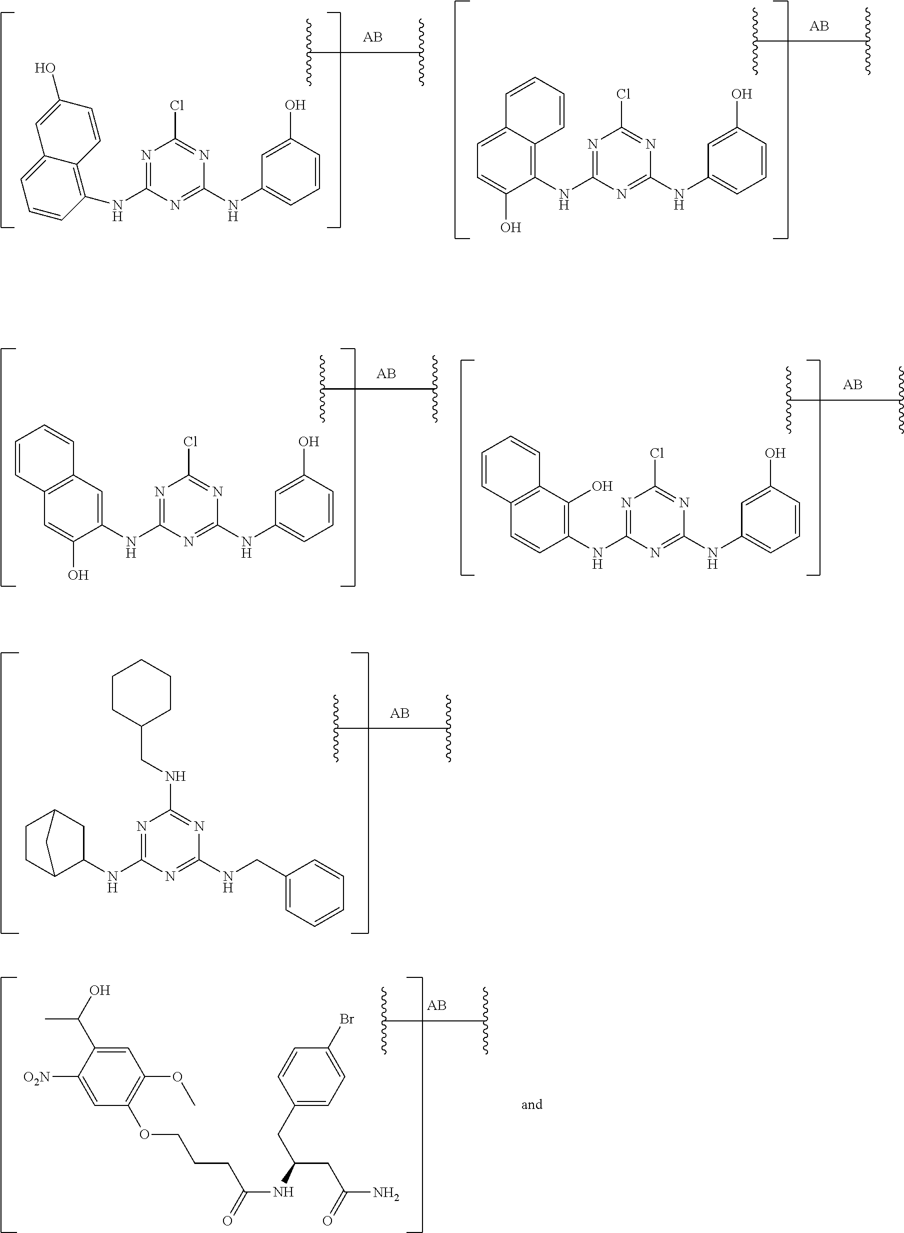

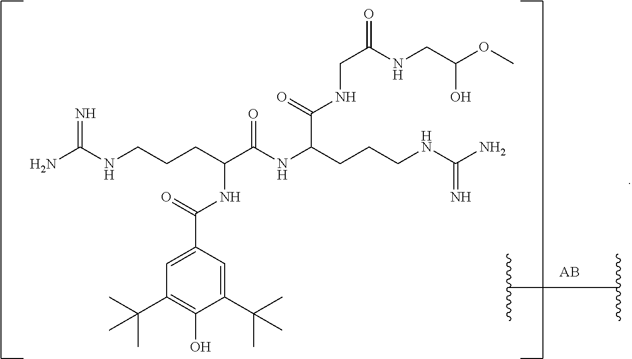

### Immunoglobulin E (IgE)

Immunoglobulin E (IgE) is a strong mediator of allergic disease, including but not limited to, atopic asthma, allergic rhinitis, atopic dermatitis, cutaneous contact hypersensitivity, IgE-mediated food allergy, IgE-mediated animal allergies, allergic conjunctivitis, allergic urticaria, anaphylactic shock, nasal polyposis, keratoconjunctivitis, mastocytosis, eosinophilic gastrointestinal disease, bullous pemphigoid, chemotherapy induced hypersensitivity reaction, seasonal allergic rhinitis, interstitial cystitis, eosinophilic esophagitis, angioedema, acute interstitial nephritis, atopic eczema, eosinophilic bronchitis, chronic obstructive pulmonary disease, gastroenteritis, hyper-IgE syndrome (Job's Syndrome), IgE monoclonal gammopathy, monoclonal gammopathy of undetermined significance (MGUS), pemphigus vulgaris, mucus membrane pemphigoid, chronic urticaria, autoimmune uveitis, rheumatoid arthritis, autoimmune pancreatitis, and allergic rhinoconjunctivitis among others.

In certain embodiments the Immunoglobulin Targeting Ligand is:

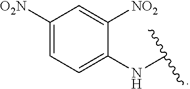 [SMILES: *Nc1ccc([N+](=O)[O-])cc1[N+](=O)[O-]]

In certain embodiments, the IgE Targeting Ligand is selected from a ligand described in Baldo, B. “IgE and Drug Allergy: Antibody Recognition of ‘Small’ Molecules of Widely Varying Structures and Activities” Antibodies 2014, 3, 56-91; Gokulrangan, G. “DNA Aptamer-Based Bioanalysis of IgE by Fluorescence Anisotropy” Anal. Chem. 2005, 77, 1963-1970; Wang, J. “Characterizing the interaction between aptamers and human IgE by use of surface plasmon resonance” Anal. Bioanal. Chem. 2008, 390:1059-1065; and US 2009/0018093

### IgM

In some embodiments the Target Extracellular Protein is IgM for example anti-MAG IgM autoantibodies. Myelin-associated glycoprotein (MAG) is a transmembrane glycoprotein that plays a role in glial-axonal interactions in the nervous system. In some patients, IgM anti-MAG antibodies develop leading to neuropathy. Antibody levels can be as high as four-fold over normal, leading to potential nephropathy. Lowering levels of anti-MAG antibodies is associated with clinical response in polyneuropathy.

Representative targeting ligands that bind to Anti-MAG IgM autoantibodies include HSO₃₋3G1cAβ1-3Ga1β1-4G1cNAcβ1-3Ga1β1-4G1cβ1-Cer; HSO₃-3G1cAβ1-3Ga1β1-4G1cNAcβ1-3Ga1β1-4G1cNAcβ1-3Ga1β1-4G1cβ1-Cer; HSO₃-3G1cAβ1-3Ga1β1-4G1cNAc-X;

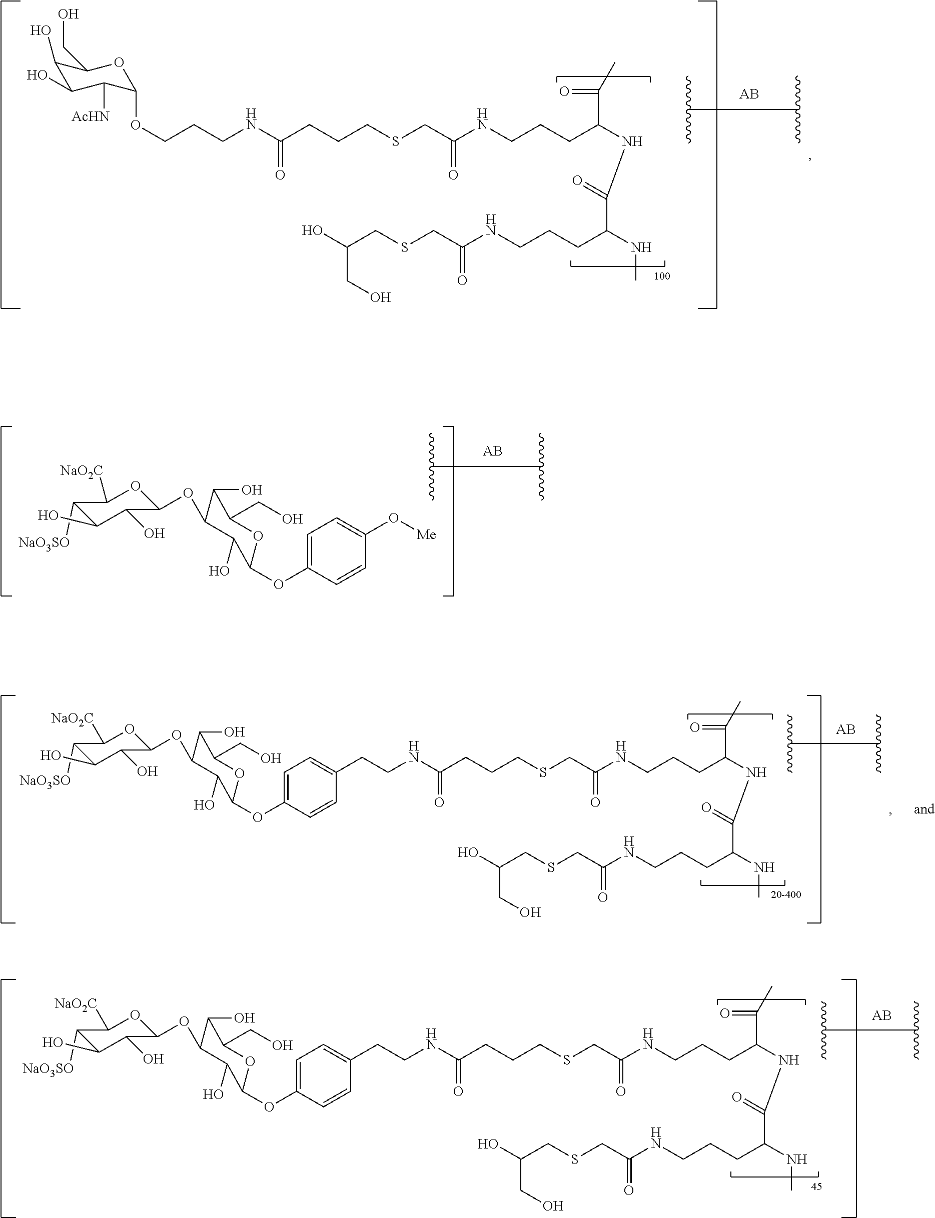

Additional ligands for IgM autoantibodies that can be used in the present invention are described in Herrendorff, R. et al. 2017 PNAS Early Edition, doi/10.1073/pnas.1619386114, WO 2018/167230, U.S. Pat. Nos. 9,056,081; 9,994,605; Volshol et al. J. Biol. Chem 1996; Wang et al. 2020; WO 2000/050447; WO 2015/136027; Bunyatov et al. “Synthetic HNK-1 containing glycans provide insight into binding properties of serum antibodies from MAG-neuropathy patients” BioRxiv, 2022; Aliu wt al. “Selective inhibition of anti-MAG IgM autoantibody binding to myelin by an antigen-specific glycopolymer” Journal of Neurochemistry 2020; WO 2022/081895; and Simon-Haldi, M. et al. “Identification of a peptide mimic of the L2/HNK-1 carbohydrate epitope” Journal of Neurochemistry 2002, 83, 1380-1388.

In certain embodiments the IgM Targeting Ligand is selected from

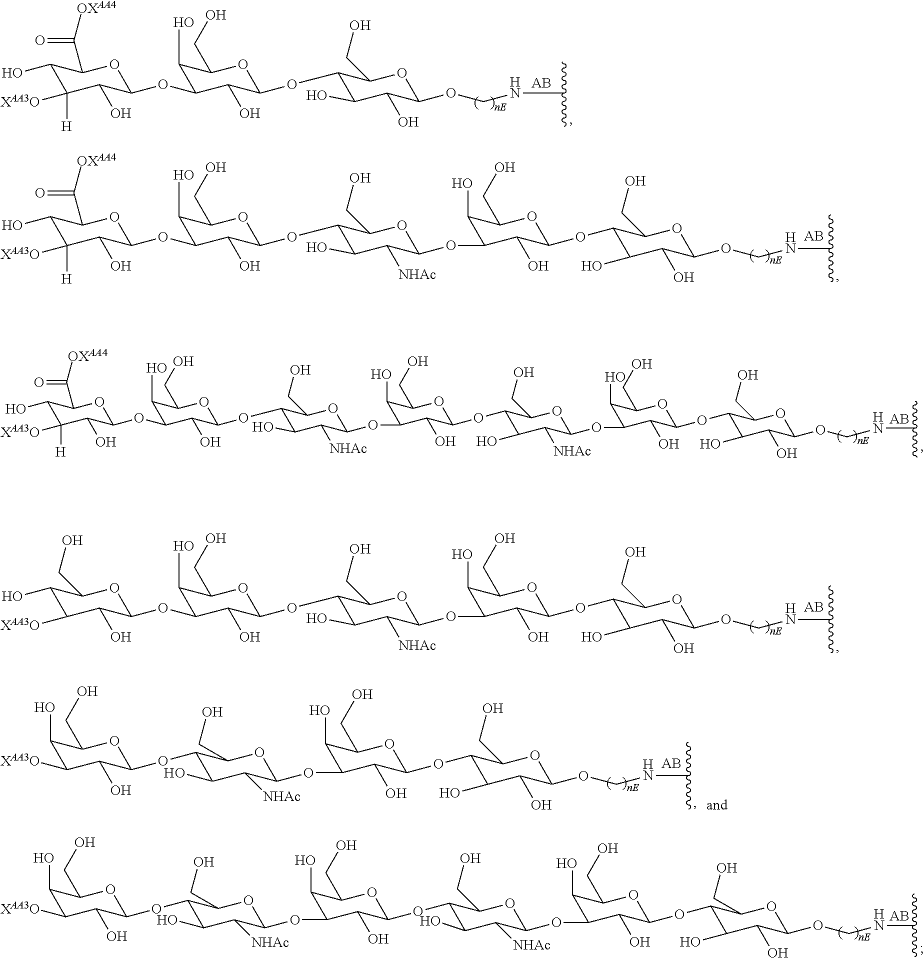

wherein
nE is 1 to 10;
X^{AA3} is selected from H, SO₃⁻, SO₃H, and SO₃Na; and
X^{AA4} is selected from H or Na.

In certain embodiments the IgM Targeting Ligand is is selected from

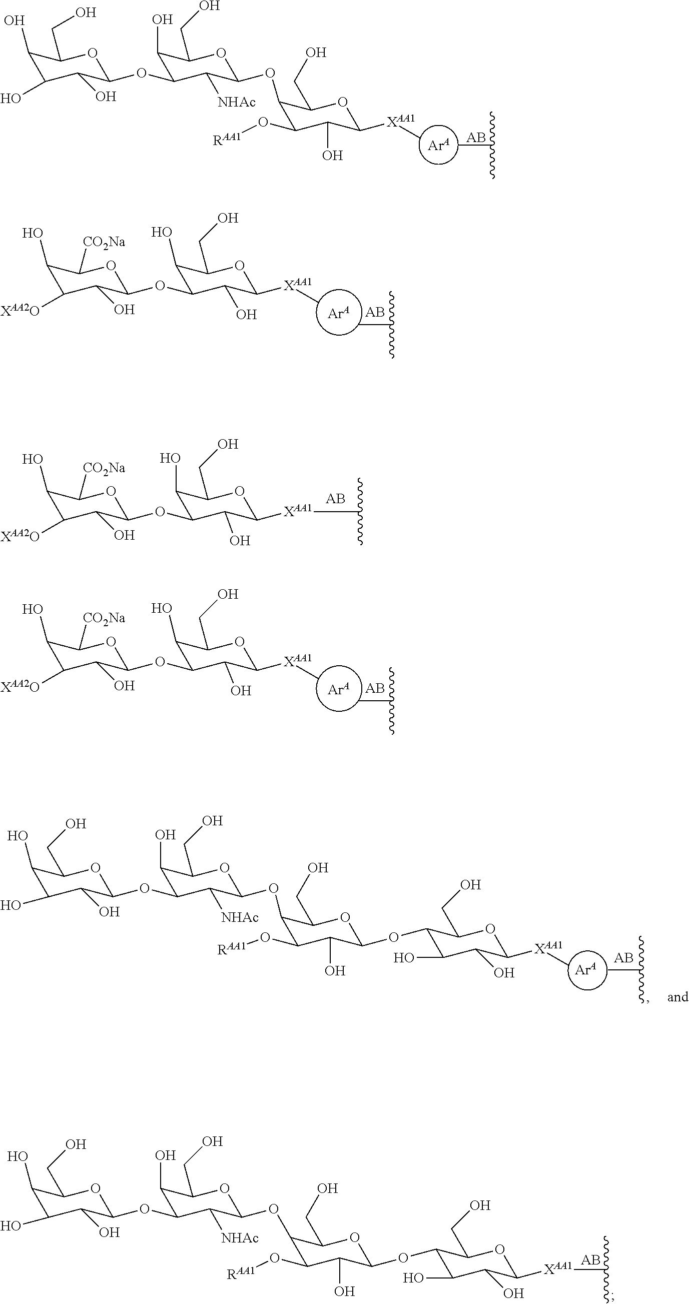

wherein
R^{AA1} is selected from a sialic acid group and an optionally substituted carboxymethyl group;
X^{AA1} is selected from O, S, NR^{AA2}, C(R^{AA2})₂;
X^{AA2} is selected from H and SO₃Na;
R^{AA2} is independently selected from H, C₁-C₄ alkyl, C₁-C₄ alkoxy, benzyl, CH₂CH₂C₆H₅, OCH₂C₆H₅, and OCH₂CH₂C₆H₅;

Ar^{A} is selected from an optionally substituted aryl and an optionally substituted heteroaryl. In certain embodiments the IgM Targeting Ligand is a compound of the formula:

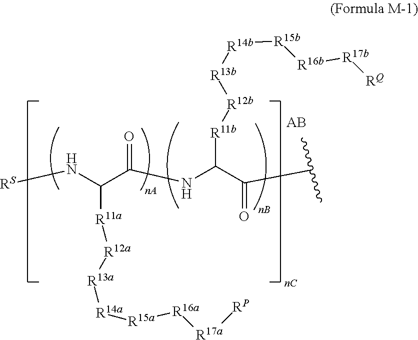

wherein
R^{11a}a, R^{11b}, R^{12a}, R^{12b}, R^{13a}, R^{13b}, R^{14a}, R^{14b}, R^{15a}, R^{15b}, R^{16a}, R^{16b}, R^{17a}, and R^{17b} are independently at each occurrence selected from the group consisting of a bond, alkyl, —C(O)—, —C(O)O—, —OC(O)—, —SO₂—, —S(O)—, —C(S)—, —C(O)NR⁶—, —NR⁹C(O)—, —O—, —S—, —NR⁶—, —C(R²¹R²¹)—, —P(O)(R³)O—, —P(O)(R³)—, a divalent residue of a natural or unnatural amino acid, alkenyl, alkynyl, haloalkyl, alkoxy, aryl, heterocycle, heteroaryl, —CH₂CH₂—[O—(CH₂)₂]ₙ—O—, —CH₂CH₂—[O—(CH₂)₂]ₙ—NR⁶—, —CH₂CH₂—[O—(CH₂)₂]ₙ—, -[—(CH₂)₂-O—]ₙ—, —[O—(CH₂)₂]ₙ—, —[O—CH(CH₃) C(O)]ₙ—, —[C(O)—CH(CH₃)—O]ₙ—, —[O—CH₂C(O)]ₙ—, —[C(O)—CH₂—O]ₙ—, a divalent residue of a fatty acid, a divalent residue of an unsaturated or saturated mono- or di-carboxylic acid, each of which is optionally substituted with 1, 2, 3, or 4 substituents independently selected from R²¹;
R^{Q} is

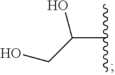 [SMILES: *C(O)CO]

W¹ is selected from H, SO₃⁻, SO₃H, and SO₃Na; and
W² is selected from H or Na;

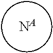

is selected from aryl, heteroaryl, and cycloalkyl;
R^{AA1} is selected from a sialic acid group and an optionally substituted carboxymethyl group;
X^{AA1} is selected from O, S, NR^{AA2}, C(R^{AA2})₂;
X^{AA2} is selected from H and SO₃Na;
R^{AA2} is independently selected from H, C₁-C₄ alkyl, C₁-C₄ alkoxy, benzyl, —CH₂-benzyl, —O-benzyl, and —O—CH₂-benzyl;
R^{S} is hydrogen, alkyl, cycloalkyl, aryl, or heteroaryl.
nA is 10-90% of the polymer and nB is 100%-nA;
nC is 1-500; and
R^{P} is selected from

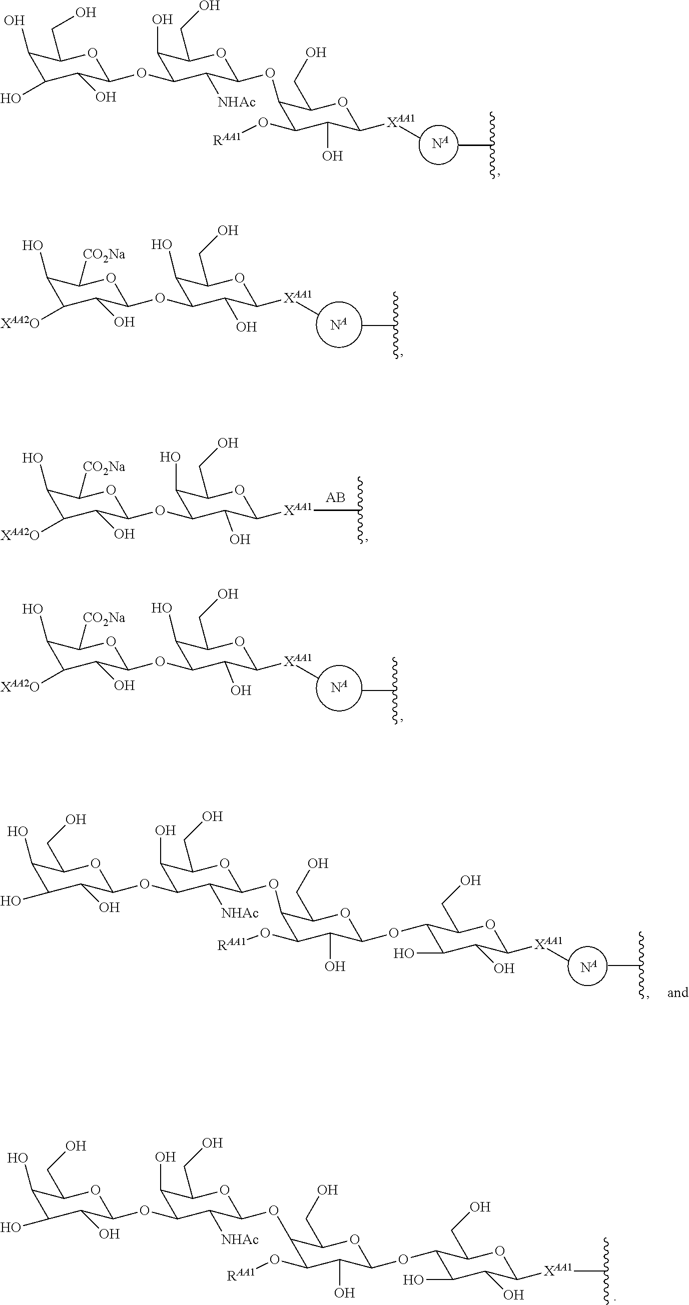

For example, a compound of the above formula is of the structure:

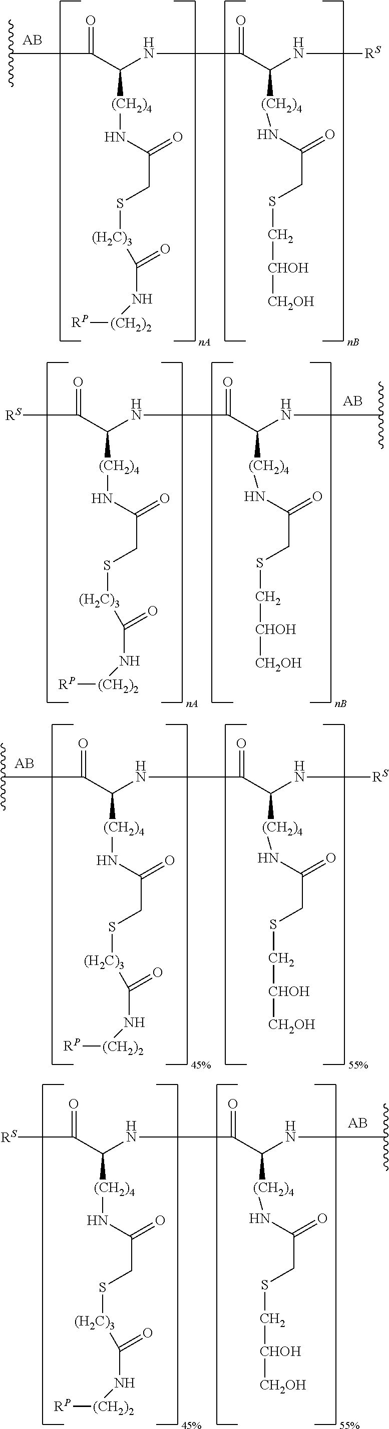

In certain embodiments of Formula M-1, nC is 150-300, 70-150, 40-60, 30-70, 15-30, or 4-5. In certain embodiments, nC is at least about 50, 60, 70, 80, 90, 100, 110, 120, 130, 140, or 150. In certain embodiments, nC is about 75, 80, 85, 90, 95, or 100.

In certain embodiments the IgM Targeting Ligand is selected from

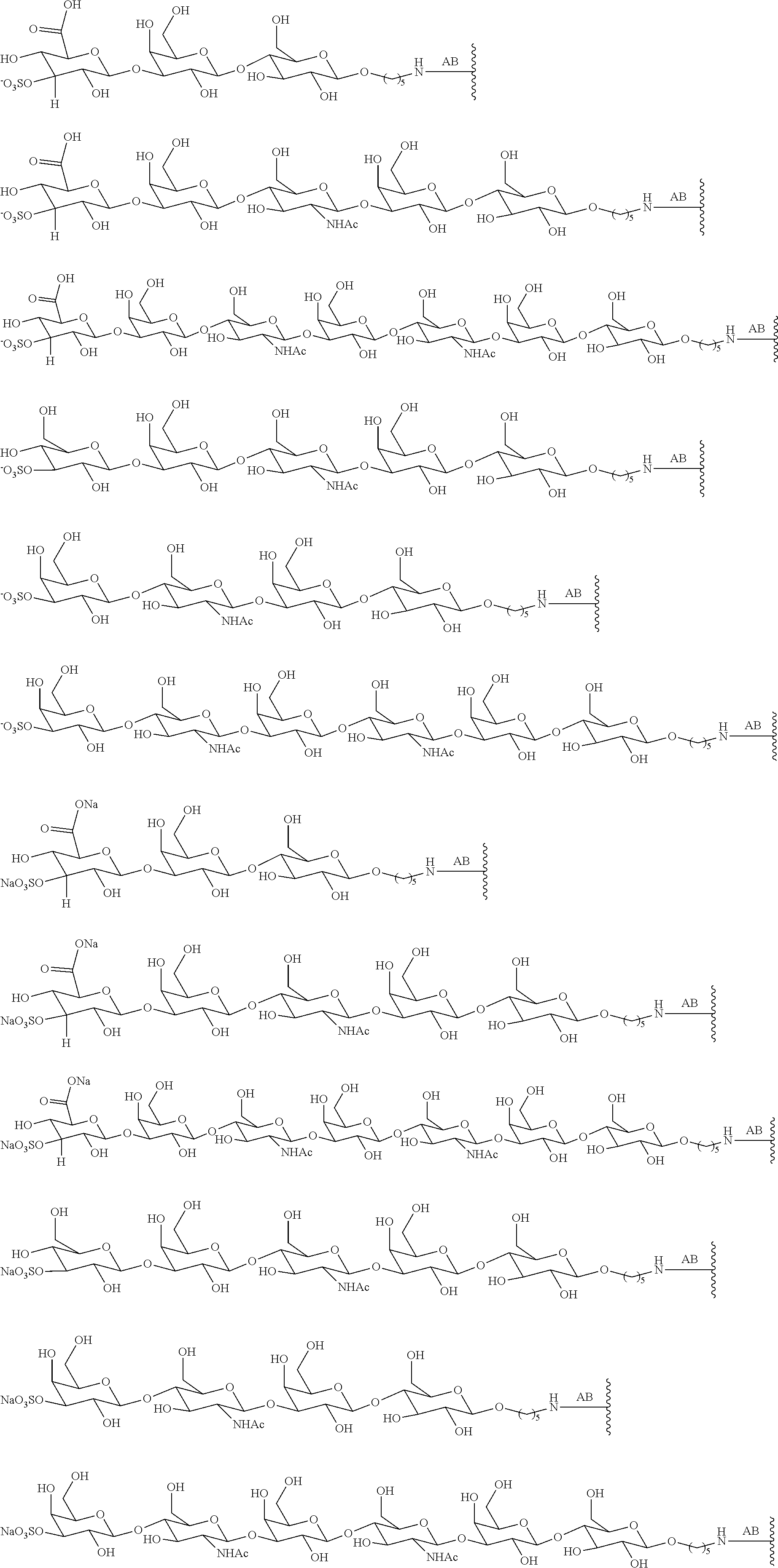

In certain embodiments the IgM Targeting Ligand is selected from

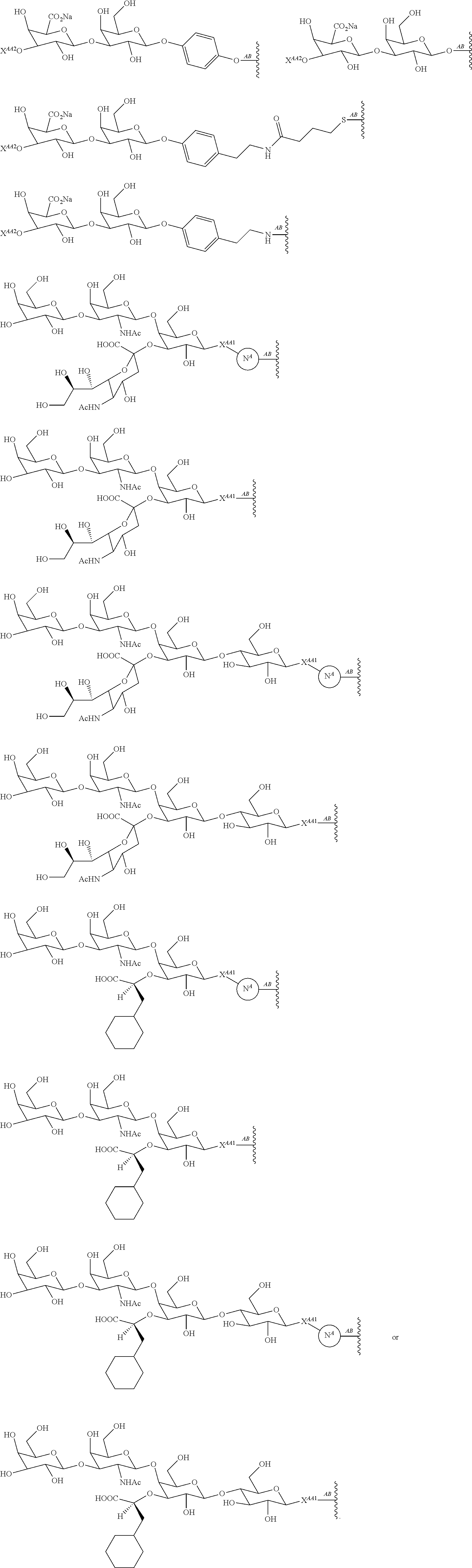

Wherein in all the above structures sodium and negative charges can be optionally replaced with hydrogen.

In certain aspects an IgM degrading compound is provided of Formula:

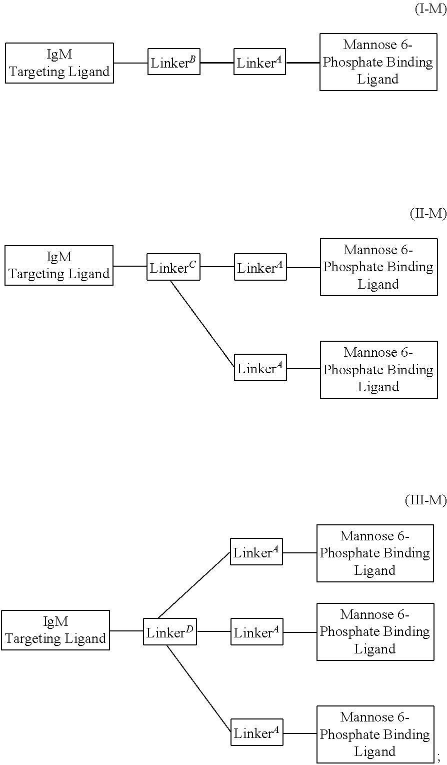

or a pharmaceutically acceptable salt thereof.

In certain embodiments the Mannose 6-Phosphate Binding Ligand used in Formula I-M, II-M, or III-M is selected from:

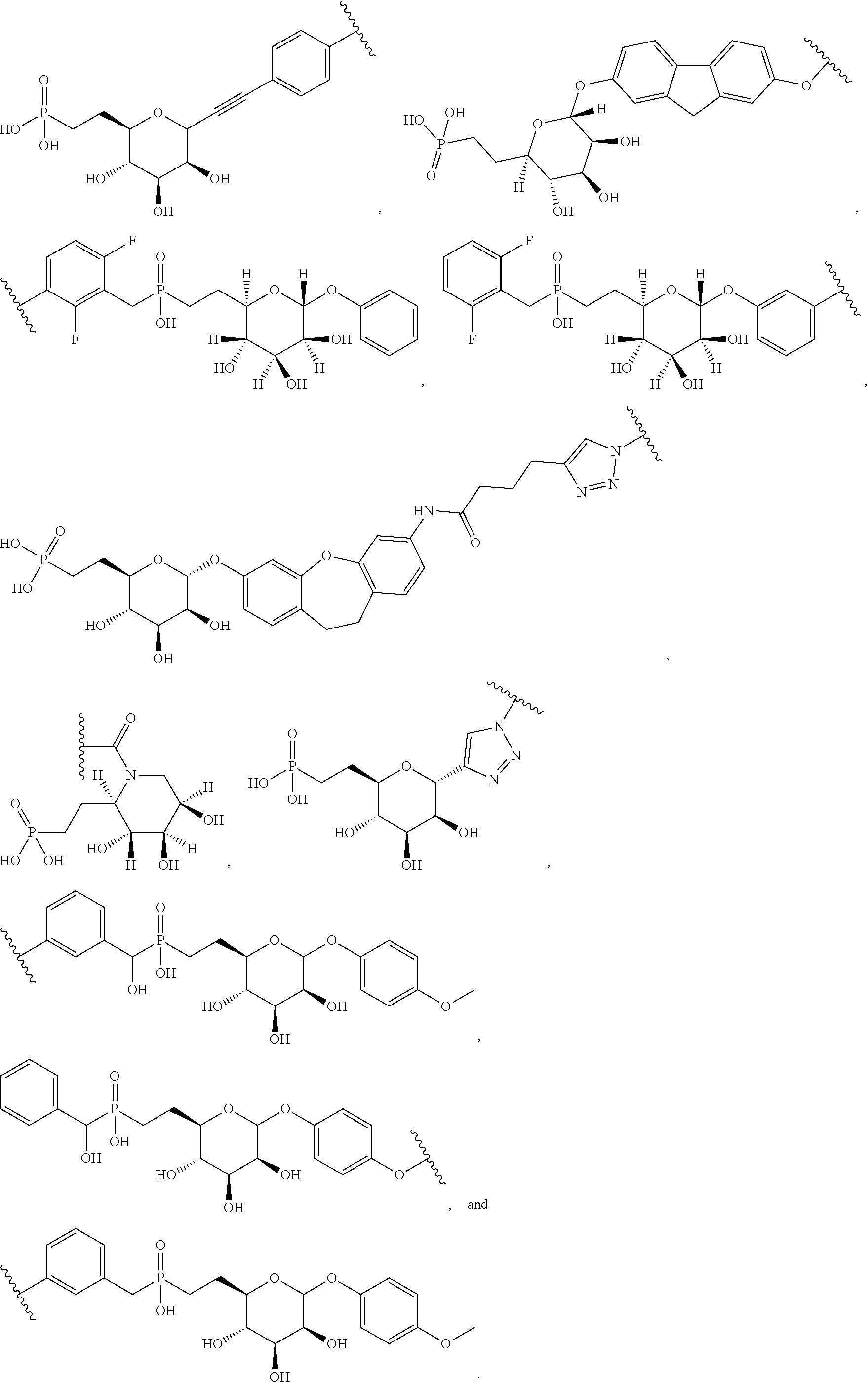

In other embodiments the Mannose 6-Phosphate Binding Ligand used in Formula I-M, II-M, or III-M is:

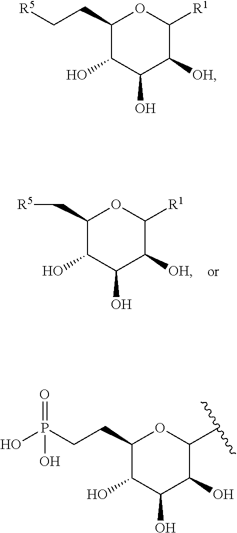

### IgE

In certain embodiments the IgE Targeting Ligand is selected from

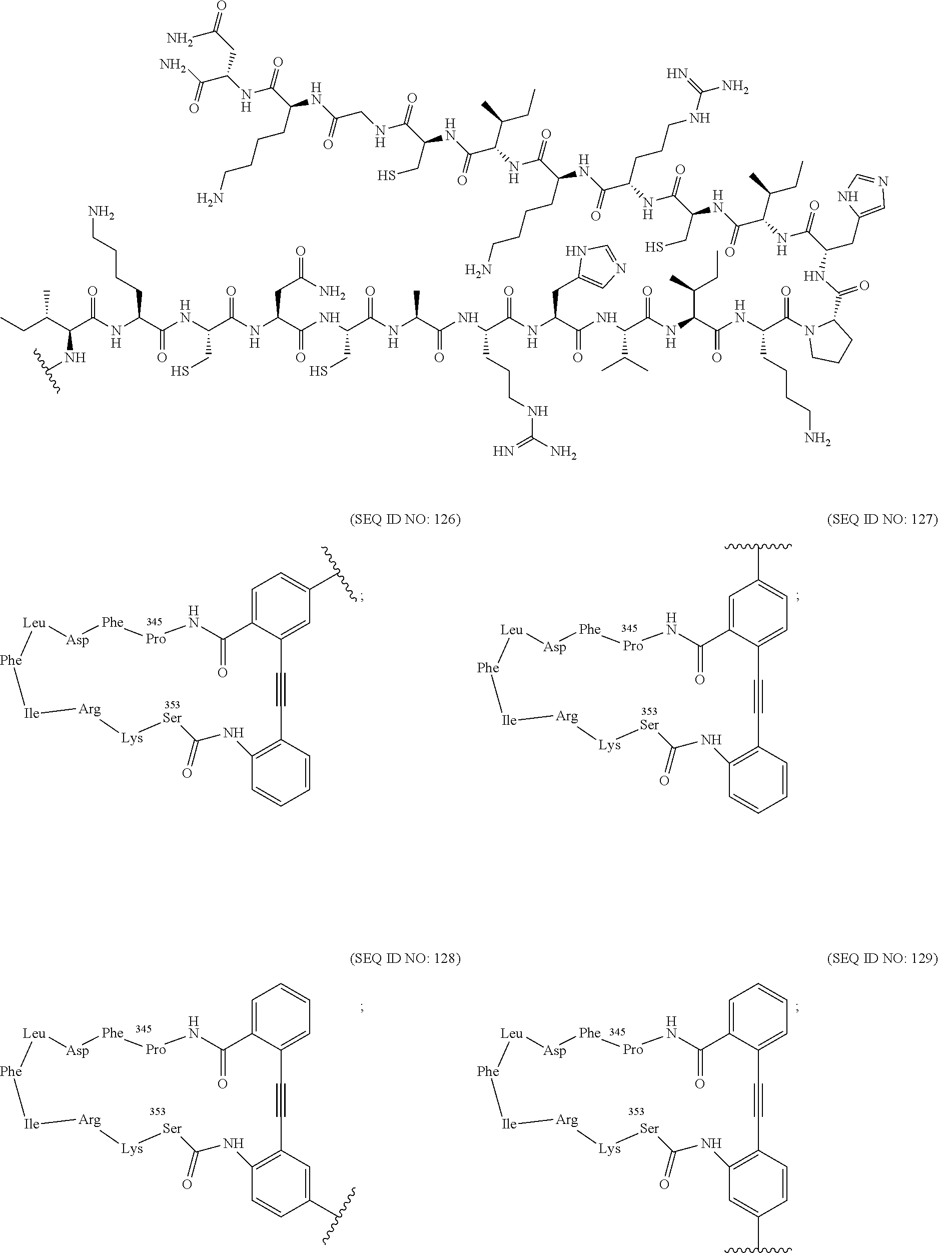

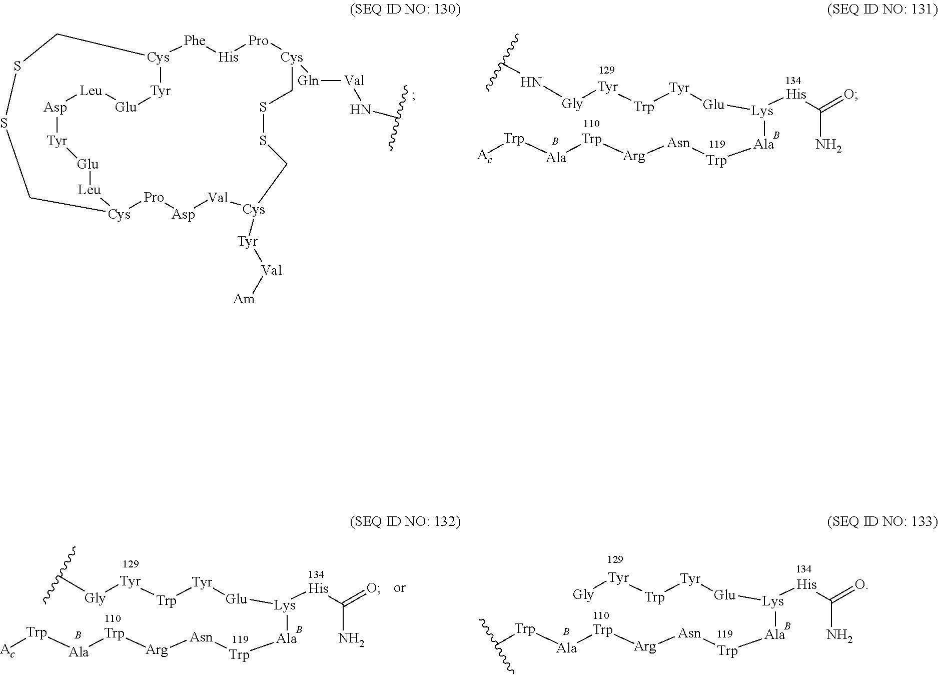

### Phospholipase A2 Receptor-1 (PLA2R) Autoantibodies

In some embodiments, the Target Extracellular Protein is an autoantibody that binds PLA2R. Phospolipase A2 Receptor-1 (PLA2R) is a major target in autoimmune membranous nephropathy. Membranous nephropathy is one of the leading causes of nephrotic syndrome, with most patients progressing to end-stage renal disease. Current treatment regimes with anti-CD20 antibodies can be ineffective at generating a complete remission. PLA2R is a transmembrane glycoprotein with a cysteine-rich N-terminal extracellular domain. This domain contains the epitope where autoantibodies bind. Reduction of autoantibody levels may provide relief to patients and complete elimination of the autoantibodies could be required to produce a durable remission.

The Protein Data Bank provides the crystal structure of the CTLD7 domain of PLA2R, the region where autoantibodies bind (6JLI; Yu et al. J. Struct. Biol. 207, 295-300). Representative PLA2R autoantibody binding ligands include, but are not limited to,

SEQ ID NO: 134
GIFVIQSESLKKC (Fresquet et al. J. Am. Soc. Nephrol 2015, 26, 302)
SEQ ID NO: 135
SVLTLENCK (Fresquet et al. J. Am. Soc. Nephrol 2015, 26, 302)
SEQ ID NO: 136
SVLTLENC (Brenchley et al. WO2019/081912)
SEQ ID NO: 137
SVLTLDNCK (Brenchley et al. WO2019/081912)
SEQ ID NO: 138
SVLTEENC (Brenchley et al. WO2019/081912)
SEQ ID NO: 139
SVLTEENS (Brenchley et al. WO2019/081912)
SEQ ID NO: 140
SVLTDENC (Brenchley et al. WO2019/081912)
SEQ ID NO: 141
SVLTDENS (Brenchley et al. WO2019/081912)
SEQ ID NO: 142
PIQSESLKK (Brenchley et al. WO2019/081912)
SEQ ID NO: 143
VIDSESLKK (Brenchley et al. WO2019/081912)
SEQ ID NO: 144
PIDSESLKK (Brenchley et al. WO2019/081912)
SEQ ID NO: 145
VIQSESLKK (Brenchley et al. WO2019/081912)
SEQ ID NO: 146
PIESES-PEG-K-PEG-SVLTEENC (Brenchley et al. WO2019/081912)
SEQ ID NO: 147
VIQSES-PEG-K-PEG-SVL TLENC (Brenchley et al. WO2019/081912)
SEQ ID NO: 148
VIQSES-PEG-K-PEG-SVL TEENC (Brenchley et al. WO2019/081912)
SEQ ID NO: 149
PIDDES-PEG-K-PEG-SVLTLENC (Brenchley et al. WO2019/081912)
SEQ ID NO: 150
PIDDES-PEG-KPEG-SVLTEENC (Brenchley et al. WO2019/081912)
SEQ ID NO: 151
VIQSESLKKCKSVLTLENC (Brenchley et al. WO2019/081912)
SEQ ID NO: 152
PIQSESLKKCKSVLTLENC (Brenchley et al. WO2019/081912)
SEQ ID NO: 153
VIESESLKKCKSVLTLENC (Brenchley et al. WO2019/081912)
SEQ ID NO: 154
VIDSESLKKCKSVLTLENC (Brenchley et al. WO2019/081912)
SEQ ID NO: 155
PIESESLKKCKSVLTLENC (Brenchley et al. WO2019/081912)
SEQ ID NO: 156
VIQSESLKKCIQAGKLENC (Brenchley et al. WO2019/081912)
SEQ ID NO: 157
PIQSESLKKCIQAGKLENC (Brenchley et al. WO2019/081912)
SEQ ID NO: 158
VIESESLKKCIQAGKLENC (Brenchley et al. WO2019/081912)
SEQ ID NO: 159
VIDSESLKKCIQAGKLENC (Brenchley et al. WO2019/081912)
SEQ ID NO: 160
PIESESLKKCIQAGKLENC (Brenchley et al. WO2019/081912)
SEQ ID NO: 161
PIQSESLKKCKSVLTLENK (Brenchley et al. WO2019/081912)
SEQ ID NO: 162
VIESESLKKCKSVLTLENK (Brenchley et al. WO2019/081912)
SEQ ID NO: 163
VIDSESLKKCKSVLTLENK (Brenchley et al. WO2019/081912)
SEQ ID NO: 164
PIESESLKKCKSVLTLENK (Brenchley et al. WO2019/081912)
SEQ ID NO: 165
VIQSESLKKCIQAGKLENK (Brenchley et al. WO2019/081912)
SEQ ID NO: 166
PIQSESLKKCIQAGKLENK (Brenchley et al. WO2019/081912)
SEQ ID NO: 167
VIESESLKKCIQAGKLENK (Brenchley et al. WO2019/081912)
SEQ ID NO: 168
VIDSESLKKCIQAGKLENK (Brenchley et al. WO2019/081912)
SEQ ID NO: 169
PIESESLKKCIQAGKLENK (Brenchley et al. WO2019/081912)
SEQ ID NO: 170
PIESESGSVLTLENCK (Brenchley et al. WO2019/081912)
SEQ ID NO: 171
PIESESGGSVLTLENCK (Brenchley et al. WO2019/081912)
SEQ ID NO: 172
PIESESGGGSVLTLENCK (Brenchley et al. WO2019/081912)
SEQ ID NO: 173
PIESESGGGGSVLTLENCK (Brenchley et al. WO2019/081912)
SEQ ID NO: 174
PIESESGGGGGSVLTLENCK (Brenchley et al. WO2019/081912)
SEQ ID NO: 175
VIQSESGSVLTLENCK (Brenchley et al. WO2019/081912)
SEQ ID NO: 176
VIQSESGGSVLTLENCK (Brenchley et al. WO2019/081912)
SEQ ID NO: 177
VIQSESGGGSVLTLENCK (Brenchley et al. WO2019/081912)
SEQ ID NO: 178
VIQSESGGGGSVLTLENCK (Brenchley et al. WO2019/081912)
SEQ ID NO: 179
VIQSESGGGGGSVLTLENCK (Brenchley et al. WO2019/081912)
SEQ ID NO: 180
KGCFVIQSESLKKSIQAGKSVLTLENCK (Brenchley et al. WO2019/081912)
SEQ ID NO: 181
LKKCIQAGKSVLTLENCKQAN (Brenchley et al. WO2019/081912)
SEQ ID NO: 182
WQDKGIFVIQSESLKKCIQAGK (Brenchley et al. WO2019/081912)
SEQ ID NO: 183
KGIFVIQSESLKKCIQAGKSVLTLENCK (Brenchley et al. WO2019/081912)
SEQ ID NO: 184
GIFVIQSESLKKC (Brenchley et al. WO2015/185949)
SEQ ID NO: 185
WSVLTLENCK (Brenchley et al. WO2015/185949)
SEQ ID NO: 186
WQDKGIFVIQSESLKKCIQAGKSVLTLENCK (Brenchley et al. WO2015/185949)
SEQ ID NO: 187
YDWIPSSAW (Glee et al., the journal of immunology, 1999, 163: 826-833)
SEQ ID NO: 188
AGAIWQRDW
SEQ ID NO: 189
AGAIWQKDW
SEQ ID NO: 190
VIQSESLK
SEQ ID NO: 191
PIQSESLK
SEQ ID NO: 192
PIESESLK
SEQ ID NO: 193
SVLTEENCK

In certain embodiments a compound is provided of Formula

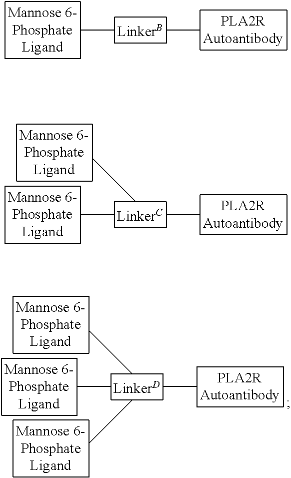

or a pharmaceutically acceptable salt thereof;
wherein

PLA2R Autoantibody is any PLA2R autoantibody described in WO2019/081912.

In certain embodiments PLA2R Autoantibody is of Formula:

SEQ ID NO: 194:
SVLTXENX;
XIXXEX;
XENXK;
SEQ ID NO: 195:
SVLTXENCK;
SEQ ID NO: 196:
XIXXEXLK;

or
or a peptide of XIXXEX, XENXK, SEQ ID No:194, 195, or 196 linked via a Linker-B group, in certain embodiments the linked sequences are SEQ ID:194 and XIXXEX or SEQ ID NO: 195 and SEQ ID NO: 196;
   wherein X is any natural amino acid or other amino acid described herein;
   and wherein the sequence is linked to a Linker described herein at a terminal amine or carboxylic acid.

### Complement C3

In some embodiments the Target Extracellular Protein is complement C3. Complement C3 is one of the major proteins involved in the complement response, a significant factor in both innate and adaptive immunity. Elevated C3 is associated with Paroxysmal nocturnal hemoglobinuria (PNH), immune complex membranoproliferative glomerulonephritis (IC-MPGN), C3 glomerulopathy (C3G), geographic (GA), age-related macular degeneration (AMD), periodontitis, amyotrophic lateral sclerosis (ALS), hematopoietic stem cell transplantation- associated thrombotic microangiopathy (HSCT-TMA), cold agglutinin disease (CAD) and host attack in gene therapies. Reduction of C3 levels may ameliorate some of the symptoms or complications that arise from these inflammatory diseases.

The Protein Data Bank website provides the crystal structure of complement C3, searchable by 2A73 (Janssen, B. J. Nature, 2005, 505-511). Complement C3 bound to a nanobody inhibitor can be found with PDB accession code 6EHG (Jensen, R. K. et al. J Biol Chem, 2018, 293, 6269-6281). Nonlimiting examples of complement C3 binding ligands include

SEQ ID NO: 197
dYI*CV(N-Me)WQDWSarAHRC*(N-Me)I
(Zhang, Y. et al. 2015,
Immunobiology, 220, 993-998)
SEQ ID NO: 198
I*CVVQDWGHHRC*TAGMANLTSHASAI,
(Sahu, A. et al. The Journal
of Immunology, 1996, 157, 884-891).
SEQ ID NO: 199
I*CVVQDWGHHRC*T,
(Sahu, A. et al. The Journal of Immunology,
1996, 157, 884-891).
SEQ ID NO: 200
*CVVQDWGHHAC*
(Sahu, A. et al. The Journal of Immunology, 1996,
157, 884-891).
SEQ ID NO: 201
(Ac)-I*CVVQDWGHHRC*T-(NH₂),
(Sahu, The Journal of Immunology,
2000, 165, 2491-2499);
SEQ ID NO: 202
*CVVQDWGHHRC*T-(NH₂),
(Sahu, The Journal of Immunology, 2000,
165, 2491-2499);
SEQ ID NO: 203
*CVVQDWGHHRC*-(NH₂),
(Sahu, The Journal of Immunology, 2000,
165, 2491-2499);
SEQ ID NO: 204
(Ac)-I*CVVGDWGHHRC*T-(NH2),
(Sahu, The Journal of Immunology,
2000, 165, 2491-2499);
SEQ ID NO: 205
(Ac)-I*CVVQPWGHHRC*T-(NH2),
(Sahu, The Journal of Immunology,
2000, 165, 2491-2499);
SEQ ID NO: 206
(Biotin)-KYSSI*CVVQDWGHHRC*T-(NH2),
(Sahu, The Journal of
Immunology, 2000, 165, 2491-2499);
SEQ ID NO: 207
(Ac)-dI*CVVQDWGHHRC*TAGHMANLTSHASAK-(Biotin),
(Sahu, The Journal of Immunology,
2000, 165, 2491-2499);
SEQ ID NO: 208
(Ac)-dI*CV(N-Me)WQDWGAHRC*T,
(Risitano et al. Blood, 2014, 123, 2094)
SEQ ID NO: 209
dYI*CV(N-Me)WQDW-Sar-AHRC*(N-Me)I,
(Risitano et al. Blood, 2014,
123, 2094)

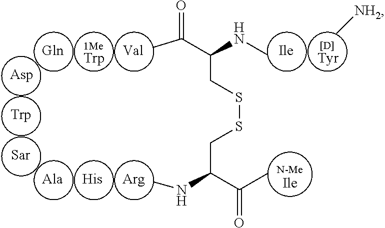

SEQ ID NO: 210
(PEG40K)-dYI*CV(N-Me)WQDW-Sar-AHRC*(N-Me)I
(Risitano et al.
Blood, 2014, 123, 2094)

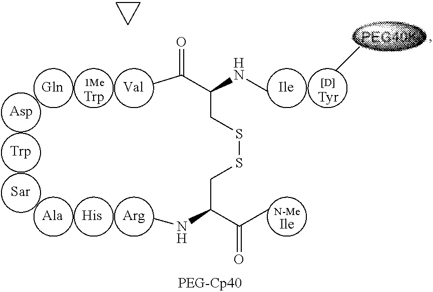

SEQ ID NO: 211:
(Ac)-dYI*CV(N-Me)WQDW-Sar-AHRC*IK

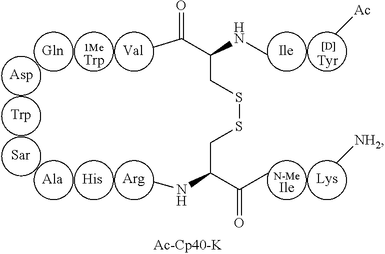

SEQ ID NO: 212:
(Ac)-dYI*CV(N-Me)WQDW-Sar-AHRC*IK-(PEG40K)

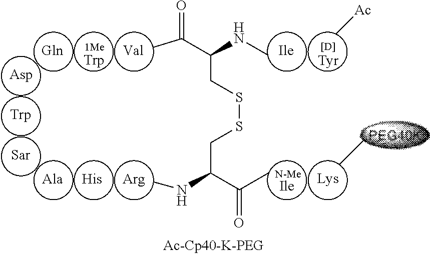

SEQ ID NO: 213
(Ac)-I*CVWQDWGAHRC*T-(NH₂)(Qu, H. et al. Immunobiology(2012)
http://dx.doi.org/10.1016/j.imbio.2012.06.003)
SEQ ID NO: 214
(Ac)I*CV(N-Me)WQDW-Sar-AHRC*I-(NH₂) (Qu, H. et al.
Immunobiology(2012) http://dx.doi.org/10.1016/j.imbio.2012.06.003)
SEQ ID NO: 215
(Ac)I*CV(N-Me)WQDW-Sar-AHRC*(N-Me)I-(NH₂) (Qu, H. et al.
Immunobiology(2012) http://dx.doi.org/10.1016/j.imbio.2012.06.003)
SEQ ID NO: 216
(Ac)I*CV(N-Me)WQDWGAHRC*T-(NH₂)
(Qu, H. et al. Molecular
Immunology, 2011, 48, 481)
SEQ ID NO: 217
(Ac)X*CVXQDWGXXXC*T-(NH₂)
(Mallik et al. J. Med. Chem., 2005,
48, 274-286)
SEQ ID NO: 218
(Ac)-I*CVVQDWGHHRC*T-(NH₂)
(Mallik et al. J. Med. Chem., 2005,
48, 274-286)
SEQ ID NO: 219
(Ac)-I*CVVQDWGAHRC*T-(NH₂)
(Mallik et al. J. Med. Chem., 2005,
48, 274-286)
SEQ ID NO: 220
(Ac)-I*CVTQDWGHHRC*T-(NH₂)
(Mallik et al. J. Med. Chem., 2005,
48, 274-286)
SEQ ID NO: 221
(Ac)-I*CVSQDWGHHRC*T-(NH₂)
(Mallik et al. J. Med. Chem., 2005,
48, 274-286)
SEQ ID NO: 222
(Ac)-I*CVHQDWGHHRC*T-(NH₂),
(Mallik et al. J. Med. Chem., 2005,
48, 274-286)
SEQ ID NO: 223
(Ac)-I*CVFQDWGHHRC*T-(NH₂),
(Mallik et al. J. Med. Chem., 2005, 48, 274-286)
SEQ ID NO: 224
(Ac)-I*CVYQDWGAHRC*T-(NH₂), (Mallik et al. J. Med. Chem., 2005,
48, 274-286)
SEQ ID NO: 225
(Ac)-I*CVWQDWGWHRC*T-(NH₂),
(Mallik et al. J. Med. Chem., 2005,
48, 274-286)
SEQ ID NO: 226
(Ac)-I*CVWQDWGHHRC*T-(NH₂),
(Mallik et al. J. Med. Chem., 2005,
48, 274-286)
SEQ ID NO: 227
(Ac)-I*CVWQDWGAHRC*T,
(Mallik et al. J. Med. Chem., 2005, 48,
274-286)
SEQ ID NO: 228
(Ac)-I*CVWQDWGAHRC*T-(NH₂),
(Mallik et al. J. Med. Chem., 2005,
48, 274-286)
SEQ ID NO: 229
(Ac)-I*CVWQDWGAdHRC*T,
(Mallik et al. J. Med. Chem., 2005, 48,
274-286)
SEQ ID NO: 230
(Ac)-I*CVWQDWGdAHRC*T,
(Mallik et al. J. Med. Chem., 2005, 48,
274-286)
SEQ ID NO: 231
(Ac)-dI*CVWQDWGAHRC*T,
(Mallik et al. J. Med. Chem., 2005, 48,
274-286)
SEQ ID NO: 232
(Ac)-I*CVWQDWGAHRC*dT,
(Mallik et al. J. Med. Chem., 2005, 48,
274-286)
SEQ ID NO: 233
(Ac)-I*CVWQDWGAHRC*T-(NH₂),
(Lopez de Victoria, A. et al. Chem
Biol Drug Des 2011, 77, 431-440)
SEQ ID NO: 234
W*CVWQDWGTNRC*W-(NH₂),
(Lopez de Victoria, A. et al. Chem Biol
Drug Des 2011, 77, 431-440)
SEQ ID NO: 235
(Ac)-D*CVWQDWGTNKC*W-(NH₂),
(Lopez de Victoria, A. et al. Chem
Biol Drug Des 2011, 77, 431-440)
SEQ ID NO: 236
Q*CVWQDWGQNQC*W-(NH₂),
(Lopez de Victoria, A. et al. Chem Biol
Drug Des 2011, 77, 431-440)
SEQ ID NO: 237
(Ac)-I*CVWQDWGAHRC*W-(NH₂),
(Lopez de Victoria, A. et al. Chem
Biol Drug Des 2011, 77, 431-440)
SEQ ID NO: 238
(Ac)-W*CVWQDWGAHRC*T-(NH₂),
(Lopez de Victoria, A. et al. Chem
Biol Drug Des 2011, 77, 431-440)
SEQ ID NO: 239
(Ac)-W*CVWQDWGAHRC*W-(NH₂), (Lopez de Victoria, A. et al. Chem
Biol Drug Des 2011, 77, 431-440)
SEQ ID NO: 240
(Ac)-I*AVWQDWGAHR-Hcy*T-(NH₂), (Knerr, P. et al. ACS Chem.
Biol., 2011, 6, 753-760)
SEQ ID NO: 241
(Ac)-I*CVWQDWGAHRC*(N-Me)I-(NH₂), (Knerr, P. et al. ACS Chem.
Biol., 2011, 6, 753-760)
SEQ ID NO: 242
(Ac)-I*AVWQDWGAHR-Hcy*(N-Me)I-(NH₂), (Knerr, P. et al. ACS
Chem. Biol., 2011, 6, 753-760)
SEQ ID NO: 243
(Ac)-I*CV(5f)WQDWGAHRC*T-(NH₂), (Katragadda et al. J. Med. Chem.
2006, 49, 4616-4622).
SEQ ID NO: 244
(Ac)-I*CV(5-Me)WQDWGAHRC*T-(NH₂), (Katragadda et al. J. Med.
Chem. 2006, 49, 4616-4622).
SEQ ID NO: 245
(Ac)-I*CV(2-Nal)QDWGAHRC*T-(NH₂), (Katragadda et al. J. Med.
Chem. 2006, 49, 4616-4622).
SEQ ID NO: 246
(Ac)-I*CVWQD(5-f)WGAHRCT-(NH₂), (Katragadda et al. J. Med. Chem.
2006, 49, 4616-4622).
SEQ ID NO: 247
(Ac)-I*CVWQD(5-Me)WGAHRCT-(NH₂), (Katragadda et al. J. Med.
Chem. 2006, 49, 4616-4622).
SEQ ID NO: 248
(Ac)-I*CVWQD(1-Me)WGAHRCT-(NH₂), (Katragadda et al. J. Med.
Chem. 2006, 49, 4616-4622).
SEQ ID NO: 249
(Ac)-I*CVYQDWGAHRC*T-(CONH₂), (WO 2021/007, 111)
SEQ ID NO: 250
(Ac)-I*CVWQDWGAHRC*T-(COOH), (WO 2021/007, 111)
SEQ ID NO: 251
(Ac)-I*CVWQDWGAHRC*T-(CONH₂), (WO 2021/007, 111)
SEQ ID NO: 252
(Ac)-I*CVWQDWGAHRC*dT-(COOH), (WO 2021/007, 111)
SEQ ID NO: 253
(Ac)-I*CV(2-Nal)QDWGAHRC*T-(CONH₂), (WO 2021/007, 111)
SEQ ID NO: 254
(Ac)-I*CV(2-Nal)QDWGAHRC*T-(COOH), (WO 2021/007, 111)
SEQ ID NO: 255
(Ac)-I*CV(1-Nal)QDWGAHRC*T-(COOH), (WO 2021/007, 111)
SEQ ID NO: 256
(Ac)-I*CV(2-lal)QDWGAHRC*T-(CONH₂), (WO 2021/007, 111)
SEQ ID NO: 257
(Ac)-I*CV(2-lal)QDWGAHRC*T-(COOH), (WO 2021/007, 111)
SEQ ID NO: 258
(Ac)-I*CVDhtQDWGAHRC*T-(COOH), (WO 2021/007, 111)
SEQ ID NO: 259
(Ac)-I*CV(Bpa)QDWGAHRC*T-(COOH), (WO 2021/007, 111)
SEQ ID NO: 260
(Ac)-I*CV(Bpa)QDWGAHRC*T-(CONH₂), (WO 2021/007, 111)
SEQ ID NO: 261
(Ac)-I*CV(Bta)QDWGAHRC*T-(COOH),
(WO 2021/007, 111)
SEQ ID NO: 262
(Ac)-I*CV(Bta)QDWGAHRC*T-(CONH₂),
(WO 2021/007, 111)
SEQ ID NO: 263
(Ac)-I*CVWQDWG(2-Abu)HRC*T-(CONH₂),
(WO 2021/007, 111)
SEQ ID NO: 264
H-GI*CVWQDWGAHRC*TAN-(COOH),
(WO 2021/007, 111)
SEQ ID NO: 265
(Ac)-I*CV(5f)WQDWGAHRC*T-(CONH₂),
(WO 2021/007, 111)
SEQ ID NO: 266
(Ac)-I*CV(5-me)WQDWGAHRC*T-(CONH₂),
(WO 2021/007, 111)
SEQ ID NO: 267
(Ac)-I*CV(1-me)WIQDWGAHRC*T-(CONH₂),
(WO 2021/007, 111)
SEQ ID NO: 268
(Ac)-I*CVWQD(5f)WGAHRC*T-(CONH₂),
(WO 2021/007, 111)
SEQ ID NO: 269
(Ac)-I*CV(5-f)WQD(5f)WGAHRC*T-(CONH₂),
(WO 2021/007, 111)
SEQ ID NO: 270
(Ac)-I*CV(5-me)WQD(5f)WGAHRC*T-(CONH₂),
(WO 2021/007, 111)
SEQ ID NO: 271
(Ac)-I*CV(1-me)WQD(5f)WGAHRC*T-(CONH₂),
(WO 2021/007, 111)
SEQ ID NO: 272
H-GI*CV(6f)WQD(6f)WGAHRC*TN-(COOH),
(WO 2021/007, 111)
SEQ ID NO: 273
(Ac)-I*CV(1-formyl)WQDWGAHRC*T-(CONH₂),
(WO 2021/007, 111)
SEQ ID NO: 274
(Ac)-I*CV(1-methoxy)WQDWGAHRC*T-(CONH₂),
(WO 2021/007, 111)
SEQ ID NO: 275
H-G I*CV(5-f)WQD(5-f)WGAHRC*TN-(COOH),
(WO 2021/007, 111)

In certain embodiments the complement C3 targeting ligand is

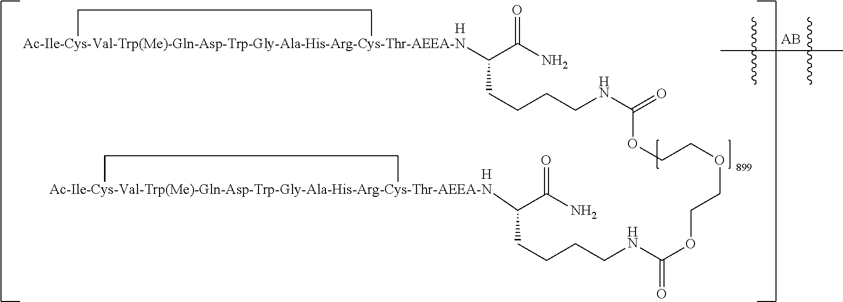

SEQ ID NO: 276
(Ac)I*CV(Me)WQDWGAHRC*T-(AEEA)- . . .

### Complement C1q

In some embodiments, the Target Extracellular Protein is Complement C1q. The complement system is part of the innate immune system and clears apoptotic cells and pathogens. Activation of this pathway begins with binding the C1 complex to an immunoglobulin that has bound to an antigen. The C1 complex consists of C1q and a tetramer of proteases (C1r and C1s). C1q mediates the binding of complement to IgG or IgM. Following the binding event, the proteases are activated, and they cleave C4 which sets off the remainder of the pathway that ends in opsonization. Overactivity of this pathway can lead to a number of inflammatory pathologies including allograft rejection, neuromyelitis optica, generalized myasthenia gravis, and cold agglutinin disease. Degradation of C1q may reduce the symptoms associated with these inflammatory diseases.

The Protein Data Bank website provides the crystal structure of Complement C1q searchable by 2JG9 (Paidassi, H. et al., J. Immunol, 2008, 180, 2329-2338), 1PK6 (Gaboriaud, C., J. Biol. Chem, 2003, (278) 46974-46982), 5HZF (Moreau, C. et al., Front. Immunol, 2016, (7) 79), 2WNV and 2WNU (Garlatti, V. et. al., J. Immunol. 2010, (185), 808). Also provided on the PDB website is the structure of complement C1q with a ligand bound, searchable by 6Z67 (Laursen, N. et al. Front. Immunol., 2020, (11), 1504)

Nonlimiting examples of complement C1q binding ligands include

SEQ ID NO: 277
(Ac)-AEAKAKA-(CONH2) (WO 88/07054)
SEQ ID NO: 278
IALILEPICCQERAA (U.S. Pat. No. 8,906,845; Sharp, J. A. et al. PLOS ONE 10(7), e0132446)
SEQ ID NO: 279
IALILEPICCQERAA-(dPEG24) (Sharp, J. A. et al. PLOS ONE 10(7), e0132446)
SEQ ID NO: 280
(dPEG24)-IALILEPICCQERAA (Sharp, J. A. et al. PLOS ONE 10(7), e0132446)
SEQ ID NO: 281
RALILEPICCQERAA (Sharp, J. A. et al. PLOS ONE 10(7), e0132446)
SEQ ID NO: 282
IRLILEPICCQERAA (Sharp, J. A. et al. PLOS ONE 10(7), e0132446)
SEQ ID NO: 283
IARILEPICCQERAA (Sharp, J. A. et al. PLOS ONE 10(7), e0132446)
SEQ ID NO: 284
IALIREPICCQERAA (Sharp, J. A. et al. PLOS ONE 10(7), e0132446)
SEQ ID NO: 285
IALILEPICCRERAA (Sharp, J. A. et al. PLOS ONE 10(7), e0132446)
SEQ ID NO: 286
IALILEPICCORRAA (Sharp, J. A. et al. PLOS ONE 10(7), e0132446)
SEQ ID NO: 287
IELILEPICCQERAA (Sharp, J. A. et al. PLOS ONE 10(7), e0132446)
SEQ ID NO: 288
IAEILEPICCQERAA (Sharp, J. A. et al. PLOS ONE 10(7), e0132446)
SEQ ID NO: 289
IALILEPICCQEEAA (Sharp, J. A. et al. PLOS ONE 10(7), e0132446)
SEQ ID NO: 290
IALILEPICCQEREA (Sharp, J. A. et al. PLOS ONE 10(7), e0132446)
SEQ ID NO: 291
IALILEEICCQERAA (Sharp, J. A. et al. PLOS ONE 10(7), e0132446)
SEQ ID NO: 292
IALILEPECCQERAA (Sharp, J. A. et al. PLOS ONE 10(7), e0132446)
SEQ ID NO: 293
PAICQRATATLGTVGSNTSGTTAIEACILL (U.S. Pat. No. 8,906,845; Sharp, J. A. et al.
Frontiers in Immunology (2014) 5, 406)
SEQ ID NO: 294
*CEGPFGPRHDLTFC*W (Roos, A. et al. The Journal of Immunology,
2001, 167, 7052)
SEQ ID NO: 295
*XbEGPFGPRHDLTFC*W (Roos, A. et al. The Journal of Immunology,
2001, 167, 7052)
SEQ ID NO: 296
QYYPFSX (Messmer B.T. et al. Molecular Immunology, 2000, 37, 343)
SEQ ID NO: 297
NPFNLAR (Messmer B.T. et al. Molecular Immunology, 2000, 37, 343)
SEQ ID NO: 298
QLQDMTSSPFWL (Messmer B.T. et al. Molecular Immunology, 2000, 37, 343)
SEQ ID NO: 299
NPFVIGRWHPPH (Messmer B.T. et al. Molecular Immunology, 2000, 37 ,343)
SEQ ID NO: 300
SLAKFLNPFLYR (Messmer B.T. et al. Molecular Immunology, 2000, 37, 343)
SEQ ID NO: 301
ASTPRFEPFQLD (Messmer B.T. et al. Molecular Immunology, 2000, 37, 343)
SEQ ID NO: 302
SLHSQPYSPFML (Messmer B.T. et al. Molecular Immunology, 2000, 37, 343)
SEQ ID NO: 303
NILSSWSSPFVF (Messmer B.T. et al. Molecular Immunology, 2000, 37, 343)
SEQ ID NO: 304
NLPSSWTNPFYL (Messmer B.T. et al. Molecular Immunology, 2000, 37, 343)
SEQ ID NO: 305
SPFMLHP (Messmer B.T. et al. Molecular Immunology, 2000, 37, 343)
SEQ ID NO: 306
PSPFMLT (Messmer B.T. et al. Molecular Immunology, 2000, 37, 343)
SEQ ID NO: 307
IGPFHLH (Messmer B.T. et al. Molecular Immunology, 2000, 37, 343)
SEQ ID NO: 308
TNPFMLN (Messmer B.T. et al. Molecular Immunology, 2000, 37, 343)
SEQ ID NO: 309
NTTFLYP (Messmer B.T. et al. Molecular Immunology, 2000, 37, 343)
SEQ ID NO: 310
SHYTQYL (Messmer B.T. et al. Molecular Immunology, 2000, 37, 343)
SEQ ID NO: 311
NHHPNYW (Messmer B.T. et al. Molecular Immunology, 2000, 37, 343)
SEQ ID NO: 312
VHYPLSW (Messmer B.T. et al. Molecular Immunology, 2000, 37, 343)
SEQ ID NO: 313
HHLKYSDTSPPI (Messmer B.T. et al. Molecular Immunology, 2000, 37, 343)
SEQ ID NO: 314
SHMHERWDTSPPI (Messmer B.T. et al. Molecular Immunology, 2000, 37, 343)
SEQ ID NO: 315
SHMHERWDTSYQ (Messmer B.T. et al. Molecular Immunology, 2000, 37, 343)
SEQ ID NO: 316
SHIHSNAAWRIT (Messmer B.T. et al. Molecular Immunology, 2000, 37, 343)
SEQ ID NO: 317
WHYPHWQ (Messmer B.T. et al. Molecular Immunology, 2000, 37, 343)
SEQ ID NO: 318
SHYLYTQ (Messmer B.T. et al. Molecular Immunology, 2000, 37, 343)
SEQ ID NO: 319
AHYSFTQ (Messmer B.T. et al. Molecular Immunology, 2000, 37, 343)
SEQ ID NO: 320
THYPTFY (Messmer B.T. et al. Molecular Immunology, 2000, 37, 343)
SEQ ID NO: 321
EHNTSFW (Messmer B.T. et al. Molecular Immunology, 2000, 37, 343)
SEQ ID NO: 322
NHYKLTW (Messmer B.T. et al. Molecular Immunology, 2000, 37, 343)
SEQ ID NO: 323
NHSPYFQ (Messmer B.T. et al. Molecular Immunology, 2000, 37, 343)
SEQ ID NO: 324
SHYQHYQ (Messmer B.T. et al. Molecular Immunology, 2000, 37, 343)
SEQ ID NO: 325
PAICQRATATLGTVGSNTSGTTEIEACILL (U.S. Pat. No. 8,906,845;
Gronemus, J.Q. et al. Molecular immunology, 2010, 48, 305)
SEQ ID NO: 326
WLGLGGGYGW (Lauvrak V., Biol. Chem. 1997, 378, 1509)
SEQ ID NO: 327
FYGPFFLNDSLRGIW (Lauvrak V., Biol. Chem. 1997, 378, 1509)
SEQ ID NO: 328
LRFLNPFSLDGSGFW (Lauvrak V., Biol. Chem. 1997, 378, 1509)
SEQ ID NO: 329
HSPFCLGVLECFGLV (Lauvrak V., Biol. Chem. 1997, 378, 1509)
SEQ ID NO: 330
TCGAFYLYHDPFICG (Lauvrak V., Biol. Chem. 1997, 378, 1509)
SEQ ID NO: 331
MQHCLASHELYLPWC (Lauvrak V., Biol. Chem. 1997, 378, 1509)
SEQ ID NO: 332
FFVFGSGDAFAFSDM (Lauvrak V., Biol. Chem. 1997, 378, 1509)
SEQ ID NO: 333
PCVIIDTGSSRWCYL (Lauvrak V., Biol. Chem. 1997, 378, 1509)
SEQ ID NO: 334
HSPFCLGVLECFGLV (Lauvrak V., Biol. Chem. 1997, 378, 1509)
SEQ ID NO: 335
HAAFEPRGDVRHTLL (Lauvrak V., Biol. Chem. 1997, 378, 1509)
SEQ ID NO: 336
CRWDGSWGEVRC (Lauvrak V., Biol. Chem. 1997, 378, 1509)
SEQ ID NO: 337
CYWVGTWGEAVC (Lauvrak V., Biol. Chem. 1997, 378, 1509)
SEQ ID NO: 338
RWFPCPNKEGCCSISV (Lauvrak V., Biol. Chem. 1997, 378, 1509)
SEQ ID NO: 339
RSTYCNKNKDSCHIPE (Lauvrak V., Biol. Chem. 1997, 378, 1509)
SEQ ID NO: 340
QPPQCIKDGGFVICRV (Lauvrak V., Biol. Chem. 1997, 378, 1509)
SEQ ID NO: 341
KGKKCKPEEHPCNEPM (Lauvrak V., Biol. Chem. 1997, 378, 1509)
SEQ ID NO: 342
NKMTCSDDGKLCWEHL (Lauvrak V., Biol. Chem. 1997, 378, 1509)
SEQ ID NO: 343
PLGRPCPTCPLAPS (Lauvrak V., Biol. Chem. 1997, 378, 1509)
SEQ ID NO: 344
QRMRPCPSCPLAPW (Lauvrak V., Biol. Chem. 1997, 378, 1509)
SEQ ID NO: 345
WPSRPCPSCPEVPP (Lauvrak V., Biol. Chem. 1997, 378, 1509)
SEQ ID NO: 346
SCTKDCPTCPLVPV (Lauvrak V., Biol. Chem. 1997, 378, 1509)
C1qNb75 Nanobody (Laursen, N. S. et al. Frontiers in Immunology, 2020, 11, 1504)
SEQ ID NO: 347
PAIAQRATATLGTVGSNTSGTTEIEACILL (U.S. Pat. No. 8,906,845)
SEQ ID NO: 348
PAICQRATATLGTVGSNTSGTTEIEAAILL (U.S. Pat. No. 8,906,845)
SEQ ID NO: 349
PAICQRATATLGTVGSNTSGTTAIEACILL (U.S. Pat. No. 8,906,845)
SEQ ID NO: 350
PAICQRATATLGTVGSNTSGTTEIAACILL (U.S. Pat. No. 8,906,845)
SEQ ID NO: 351
PAICQRAEIEACILL (U.S. Pat. No. 8,906,845)
SEQ ID NO: 352
PAIAQRAEIEAAILL (U.S. Pat. No. 8,906,845)
SEQ ID NO: 353
PAICQRATATLGTNTSGTTEIEACILL (U.S. Pat. No. 8,906,845)
SEQ ID NO: 354
PAICQRATATLSGTTEIEACILL (U.S. Pat. No. 8,906,845)
SEQ ID NO: 355
PAICQRATATTEIEACILL (U.S. Pat. No. 8,906,845)
SEQ ID NO: 356
AICQRATATLGTVGSNTSGTTEIEACILL (U.S. Pat. No. 8,906,845)
SEQ ID NO: 357
ICQRATATLGTVGSNTSGTTEIEACILL (U.S. Pat. No. 8,906,845)
SEQ ID NO: 358
CQRATATLGTVGSNTSGTTEIEACILL (U.S. Pat. No. 8,906,845)
SEQ ID NO: 359
PAICQRATATLGTVGSNTSGTTEIEACIL (U.S. Pat. No. 8,906,845)
SEQ ID NO: 360
PAICQRATATLGTVGSNTSGTTEIEACI (U.S. Pat. No. 8,906,845)
SEQ ID NO: 361
PAICQRATATLGTVGSNTSGTTEIEAC (USU.S. Pat. No. P 8,906,845)
SEQ ID NO: 362
(Ac)-IALILEPICCQERAA (U.S. Pat. No. 8,906,845)
SEQ ID NO: 363
(Ac)-PAICQRATATLGTVGSNTSGTTEIEACILL (U.S. Pat. No. 8,906,845)
SEQ ID NO: 364
(Ac)-PAIAQRATATLGTVGSNTSGTTEIEACILL (U.S. Pat. No. 8,906,845)
SEQ ID NO: 365
(Ac)-PAICQRATATLGTVGSNTSGTTEIEAAILL (U.S. Pat. No. 8,906,845)
SEQ ID NO: 366
(Ac)-PAICQRATATLGTVGSNTSGTTAIEACILL (U.S. Pat. No. 8,906,845)
SEQ ID NO: 367
(Ac)-PAICQRATATLGTVGSNTSGTTEIAACILL (U.S. Pat. No. 8,906,845)
SEQ ID NO: 368
(Ac)-PAICQRAEIEACILL (U.S. Pat. No. 8,906,845)
SEQ ID NO: 369
(Ac)-PAIAQRAEIEAAILL (U.S. Pat. No. 8,906,845)
SEQ ID NO: 370
(Ac)-PAICQRATATLGTNTSGTTEIEACILL (U.S. Pat. No. 8,906,845)
SEQ ID NO: 371
(Ac)-PAICQRATATLSGTTEIEACILL (U.S. Pat. No. 8,906,845)
SEQ ID NO: 372
(Ac)-PAICQRATATTEIEACILL (U.S. Pat. No. 8,906,845)
SEQ ID NO: 373
(Ac)-ICQRATATLGTVGSNTSGTTEIEACILL (U.S. Pat. No. 8,906,845)
SEQ ID NO: 374
(Ac)-CQRATATLGTVGSNTSGTTEIEACILL (U.S. Pat. No. 8,906,845)
SEQ ID NO: 375
(Ac)-PAICQRATATLGTVGSNTSGTTEIEACIL (U.S. Pat. No. 8,906,845)
SEQ ID NO: 376
(Ac)-PAICQRATATLGTVGSNTSGTTEIEACI (U.S. Pat. No. 8,906,845)
SEQ ID NO: 377
(Ac)-PAICQRATATLGTVGSNTSGTTEIEAC (U.S. Pat. No. 8,906,845)

In certain embodiments the Linker is bound through the C-terminus of the amino acid sequence for example

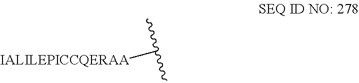

In certain embodiments the Linker is bound to the N-terminus for example

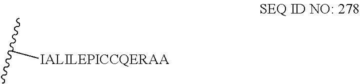

In certain embodiments the complement C3 Targeting Ligand is selected from:

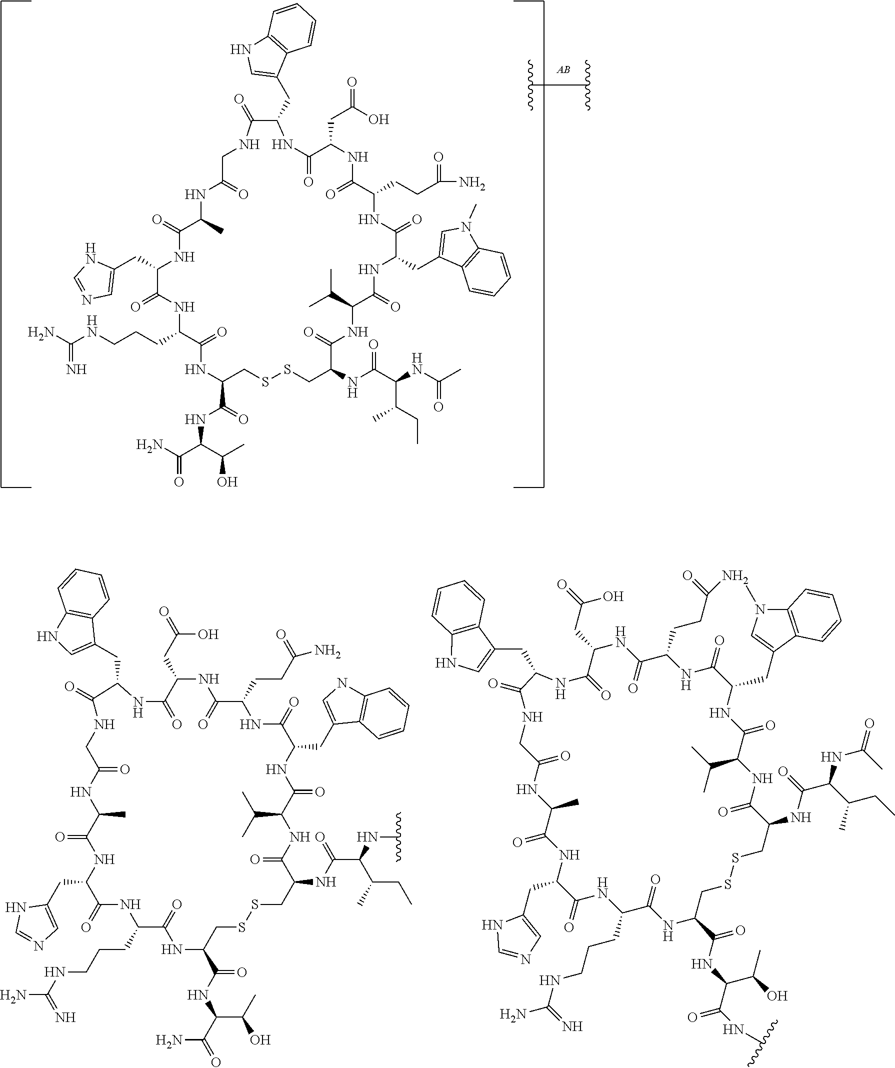

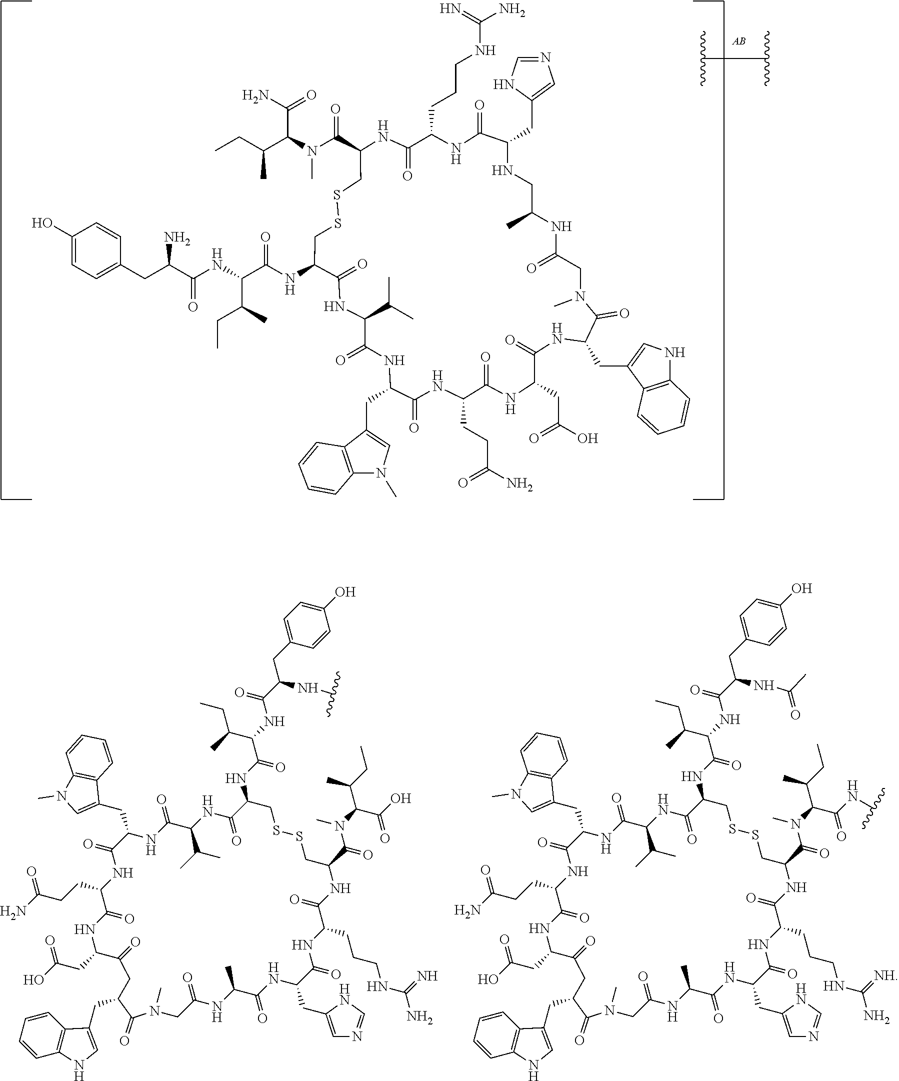

### IL-17

In some embodiments, the Target Extracellular Protein is human interleukin-17 (IL-17) (UniProtKB-Q16552 (IL17_HUMAN)). Interleukin-17 is a 35 kDa homodimeric glycoprotein and is an important cytokine for the inflammatory response. IL-17 is secreted by a distinct class of Helper T cells (known as Th17 cells) which mediates tissue inflammation. A characteristic effect of IL-17 production is the expansion of neutrophils, and in healthy tissue it is responsible for neutrophil homeostasis. IL-17 has been implicated as a major factor in psoriasis as well as other autoimmune diseases. Other diseases where IL-17 therapies may be of benefit include but are not limited to asthma, rheumatoid arthritis, psoriatic arthritis, Crohn's disease, and inflammatory bowel disease. Inflammation caused by IL-17 has been shown to hamper recovery post-stroke.

The Protein Data Bank website provides the crystal structure of IL-17, searchable by 4NUX (Zhang, B. et al. (2014) Acta Crystallogr D Biol Crystallogr 70:1476-1483), 4HSA (Liu, S. et al. (2013) Nat Commun 4:1888-1888), 4QHU (unpublished), 6WIR (Lieu, R. et al. (2020) PLoS One 15: e0232311-e0232311), 5VB9 (Ting, J. P. et al. (2018) PLoS One 13: e0190850-e0190850), 4NUX (Zhang, et al. (2014) Acta Crystallogr D Biol Crystallogr 70:1476-1483), 3JVF (Ely, L. K. et al. (2009) Nat Immunol 10:1245-1251), 5N9B (unpublished), 2VXS (Gerhardt, S. et al. (2009) J Mol Biol 394:905).

Non-limiting examples of IL-17 Targeting Ligands can be found in, for example, WO2012101263A1, WO2020163554A1, WO2021055376A1, WO2020146194A1, WO2020127685A1, US20150005319, WO2014066726A2, WO2019223718A1, WO2020135872A1, WO2020146194A1, WO2021027721A1, WO2021027724, WO2021027729A1, WO2021067191A1, CN104069102A, CN105601617B, CN108299256B, Liu et al. “Binding site elucidation and structure guided design of macrocyclic IL-17A antagonists” 2016, Scientific Reports, 6:30859., Liu et al. “Inhibiting complex IL-17AA and IL-17RA interactions with a linear peptide” 2016, Scientific Reports 6:26071. Wang, W. et al. “Artificial macrocycles as IL-17A/IL-17RA antagonists”. Med. Chem. Comm. 2018, 9, 22. Liu, C. et al. “The flavonoid cyanidin blocks binding of the cytokine interleukin-17A to the IL-17RA subunit to alleviate inflammation in vivo” Science Signaling 10, eaaf8823 (2017).

Additional binding ligands include

SEQ ID NO: 378
IVVTAPADLWDWIRA
(Liu et al. 2016, Scientific Reports 6: 26071)
SEQ ID NO: 379
ITVTMPADLWDWIRA
(Liu et al. 2016, Scientific Reports 6: 26071)
SEQ ID NO: 380
IVVTIPADLWDWIRA
(Liu et al. 2016, Scientific Reports 6: 26071)
SEQ ID NO: 381
IVVTLPADLWDWIRA
(Liu et al. 2016, Scientific Reports 6: 26071)
SEQ ID NO: 382
IVVTVPADLWDWIRA
(Liu et al. 2016, Scientific Reports 6: 26071)
SEQ ID NO: 383
IVVTMPADLWDWIMA
(Liu et al. 2016, Scientific Reports 6: 26071)
SEQ ID NO: 384
IVVTMPADLWDWINA
(Liu et al. 2016, Scientific Reports 6: 26071)
SEQ ID NO: 385
IVVTMPADLWDWIQA
(Liu et al. 2016, Scientific Reports 6: 26071)
SEQ ID NO: 386
IHVTIPADLWDWINK
(Liu et al. 2016, Scientific Reports 6: 26071)
SEQ ID NO: 387
IHVTIPADLWDWIN
(Liu et al. 2016, Scientific Reports 6: 26071)

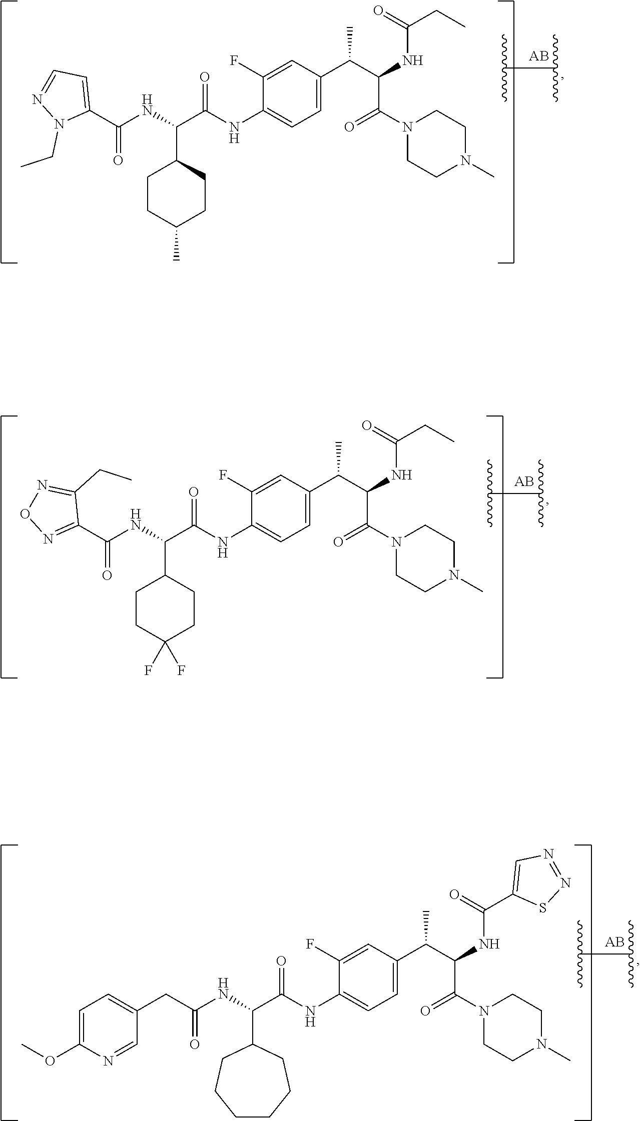

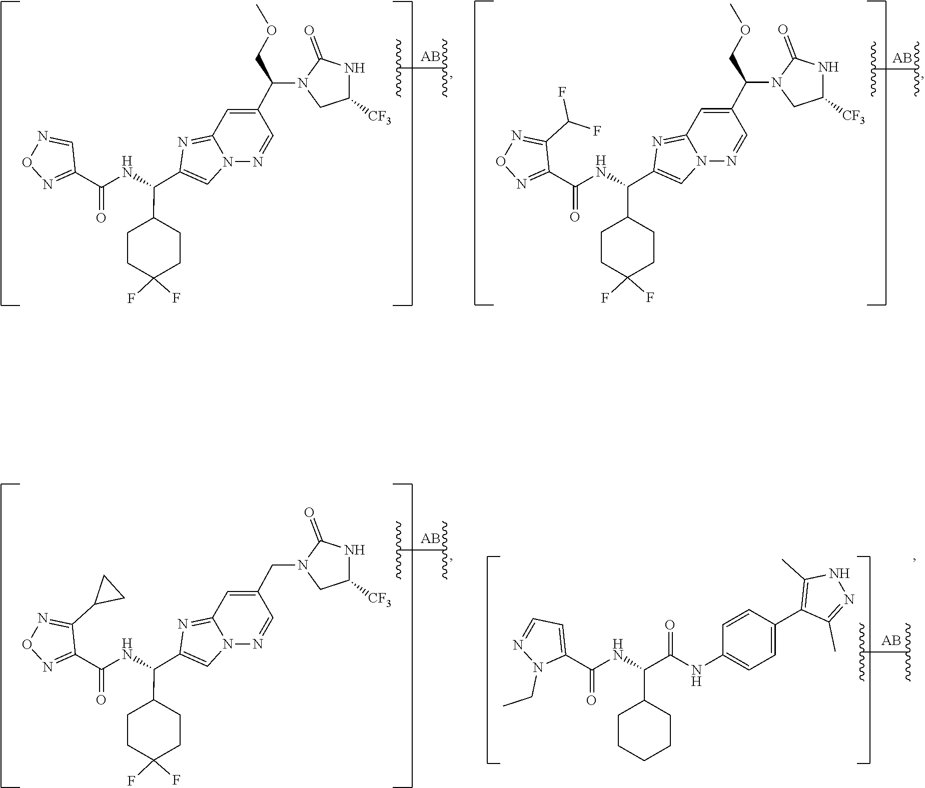

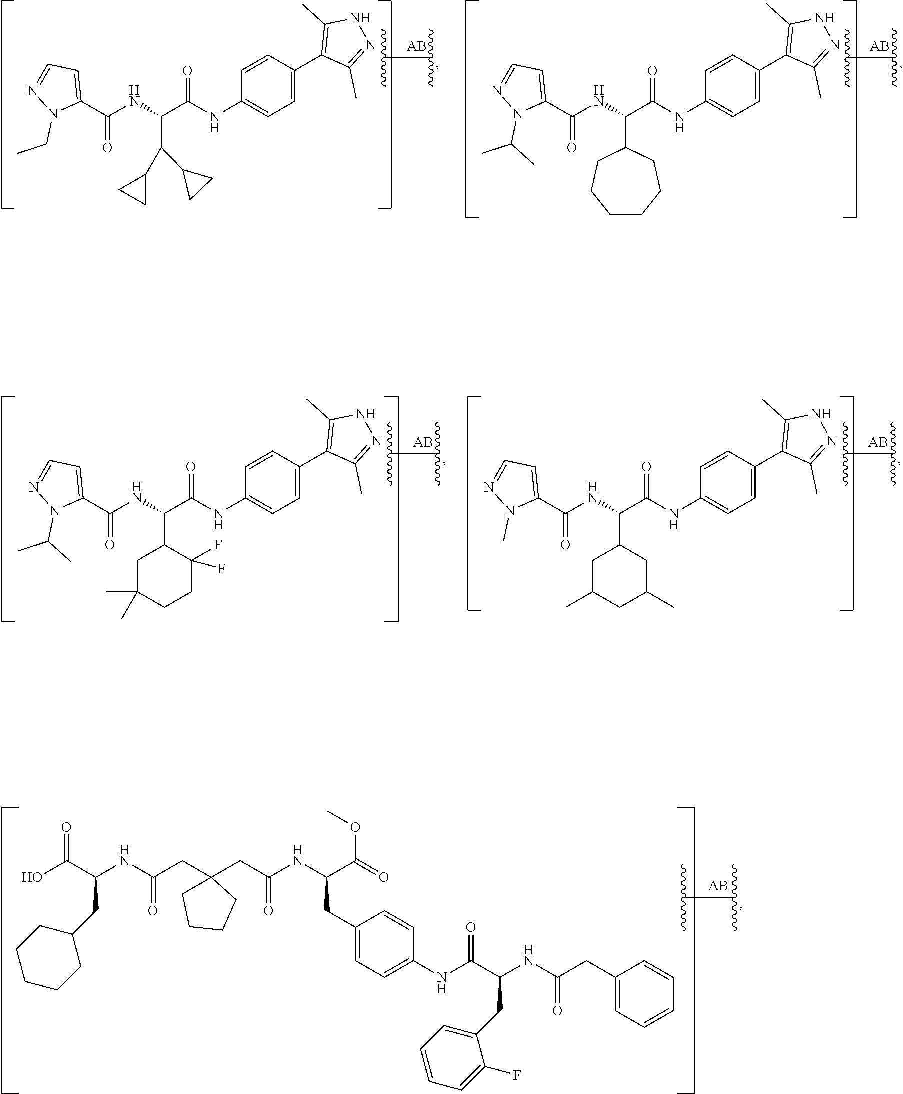

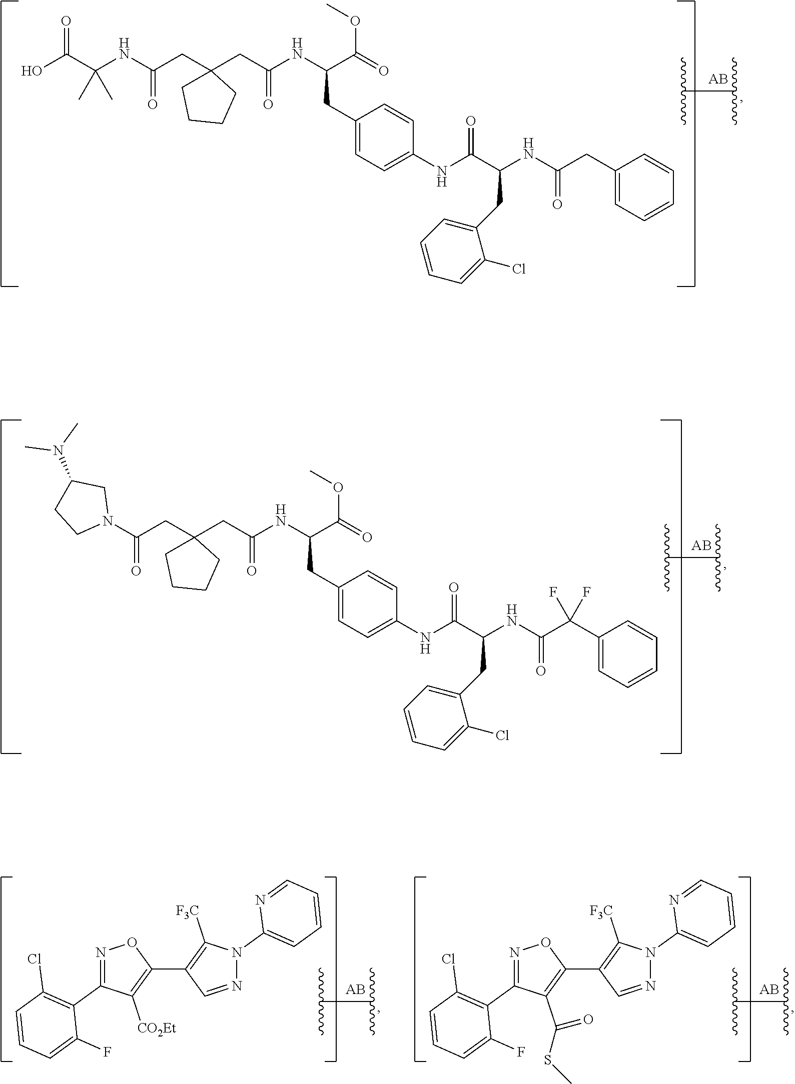

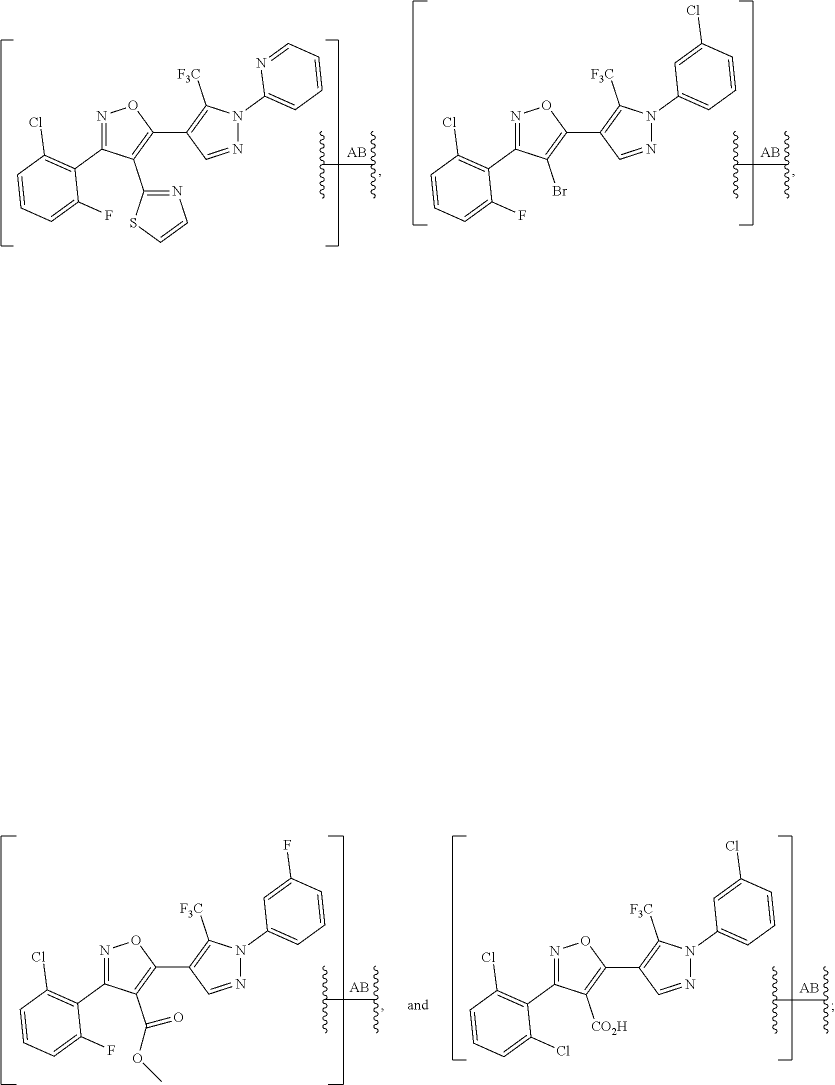

each of which is optionally substituted with 1, 2, 3, or 4 substituents independently selected from R²¹.

In certain embodiments a compound is provided of Formula

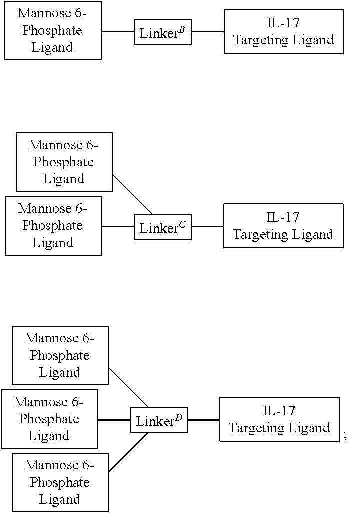

or a pharmaceutically acceptable salt thereof;
wherein

IL-17 Targeting Ligand is any IL-17 ligand described in WO2020/146,194; WO2020/163,554; WO2020/127,685; and WO2021/055,376; each of which is incorporated by reference.

In certain embodiments IL-17 Targeting Ligand is of Formula:

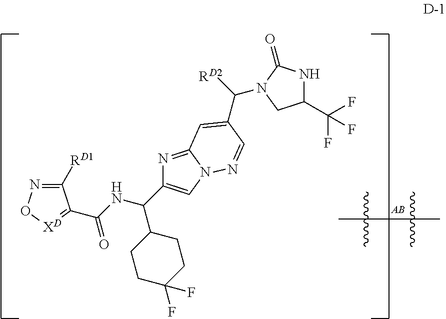

wherein,
X^{D} is CH or N;
R^{D1} is —CH₃, —CH₂F, —CHF₂, —CF₃, —CH₂CH₃, —CH₂CF₃, —CH(CH₃)₂, CH₂CHF₂, CH₂CH₂F, —CF(CH₃)₂, CF₂CH₃, —OCH₃,

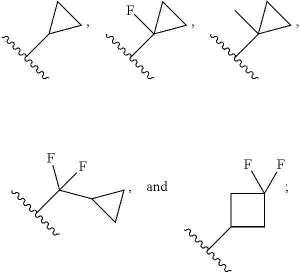

R^{D2} is —H or —CH₂OCH₃.

In certain embodiments IL-17 Targeting Ligand is of Formula:

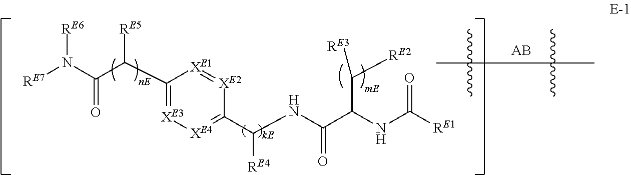

wherein;

R^{E1} is alkyl, substituted alkyl, aryl, substituted aryl, heteroaryl, substituted heteroaryl, arylalkyl, substituted arylalkyl, heteroarylalkyl, substituted heteroarylalkyl-OR^{E8} or —NR^{E9}R^{E10} or an F pocket substituent;
R^{E2} is alkyl, substituted alkyl, heterocycle, substituted heterocycle, aryl, substituted aryl, fused cycloalkylaryl, substituted fused cycloalkylaryl, heteroaryl, substituted heteroaryl or a D pocket substituent;
each R^{E3} is independently hydrogen, (C₁-C₇)alkyl, (C₁-C₇) substituted alkyl or —OR^{E32}, m^{E} is 0, 1 or 2;
each R^{E4} is independently hydrogen, (C₁-C₇)alkyl, (C₁-C₇) substituted alkyl, cycloalkyl substituted cycloalkyl, heterocycle or substituted heterocycle;
k^{E} is 0 or 1;
X^{E1}, X^{E2}, X^{E3} and X^{E4} are independently —N—or—CR^{E11}-provided that no more than two of X^{E1} X^{E2} X^{E3} and X^{E4} are nitrogen;
each R^{E5} is independently hydrogen, (C₁-C₇)alkyl, (C₁-C₇) substituted alkyl, heterocycle, substituted heterocycle, cycloalkyl, substituted cycloalkyl, heterocyclealkyl, substituted heterocyclealkyl, —NR^{E12}R^{E13}, —NR^{E14}C(O)R^{E15}, —NHSO₂R^{E31}, OH or a B pocket substituent; R^{E6} is hydrogen or alkyl;
R^{E7} is heterocycle, substituted heterocycle, —(CHR^{E16})ₒR^{E17} or —(CHR^{E18})ₚR^{E19} or R^{E6} and R^{E7} taken together with the nitrogen atom to which they are attached form piperazine, substituted piperazine, heterocycle or substituted heterocycle,

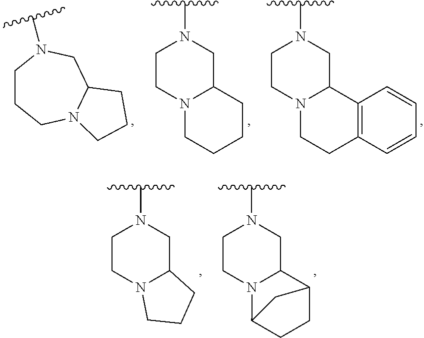

or an A pocket substituent;
R^{E8} is (C₁-C₇)alkyl, (C₁-C₇) substituted alkyl, aryl, substituted aryl, heteroaryl or substituted heteroaryl;
each R^{E11} is independently hydrogen, alkyl, substituted alkyl, —OR^{E20}, —NR^{E21}R^{E22}, halo, —CN, —CO2R^{E23}, —CONR^{E24}R^{E25}, or —SR^{E26},
n^{E} is 1, 2 or 3;
o^{E} is 1, 2 or 3;
p^{E} is 1, 2 or 3;
each R^{E16} is independently hydrogen, (C₁-C₇)alkyl or (C₁-C₇) substituted ALKYL
R^{E17} is

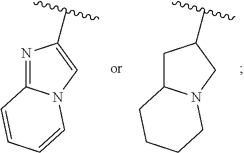 [SMILES: *c1cn2ccccc2n1 ; *C1CC2CCCCN2C1]

each R^{E18} is independently hydrogen, (C₁-C₇)alkyl, or (C₁-C₇) substituted alkyl;
R^{E19} is —NR^{E27}R^{E28};
R^{E27} and R^{E28} together with the nitrogen atom to which they are attached form a heterocycle or substituted heterocycle ring or

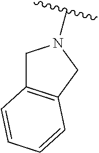 [SMILES: *N1Cc2ccccc2C1]

R^{E9}, R^{E10}, R^{E12}, R^{E13}, R^{E14}, R^{E1}S, R^{E18}, R^{E1}9, R^{E20}, R^{E21}, R^{E22}, R^{E23}, R^{E24}, R^{E25}, R^{E26}, R^{E30}, R^{E31}, and R^{E32} are independently selected at each instance from hydrogen, alkyl, substituted alkyl, heterocycle, substituted heterocycle, aryl, substituted aryl, heteroaryl, substituted heteroaryl, or alternatively, independently, R^{E9} and R^{E10}, R^{E21} and R^{E22} and R^{E24} and R^{E25} together with atom to which they are attached form a cycloalkyl, substituted cycloalkyl, heterocycle or substituted heterocycle ring;
R^{E28} is hydrogen or alkyl;
A-pocket substituent is selected from the group consisting of

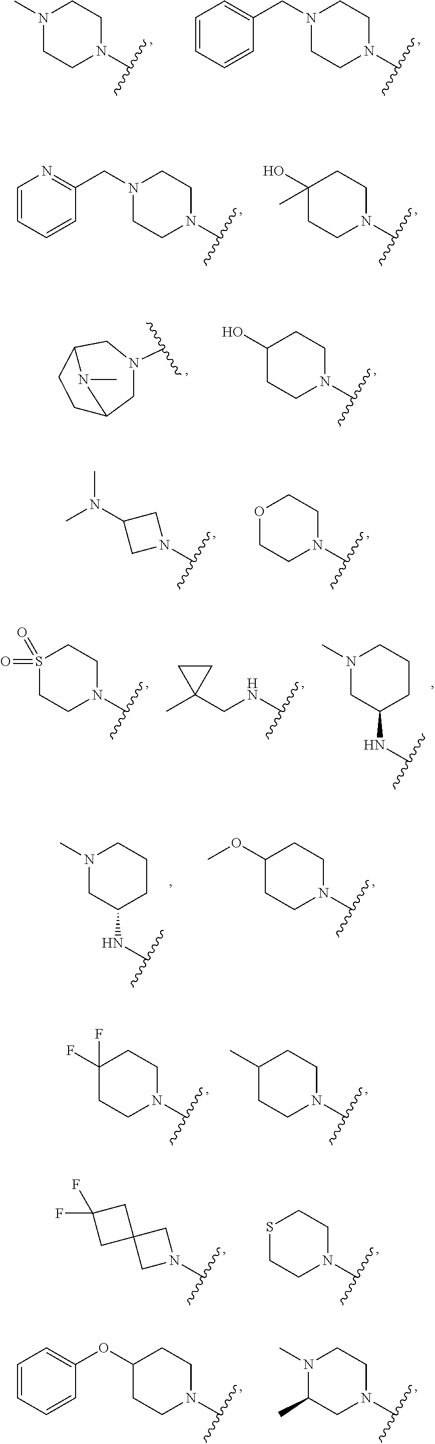

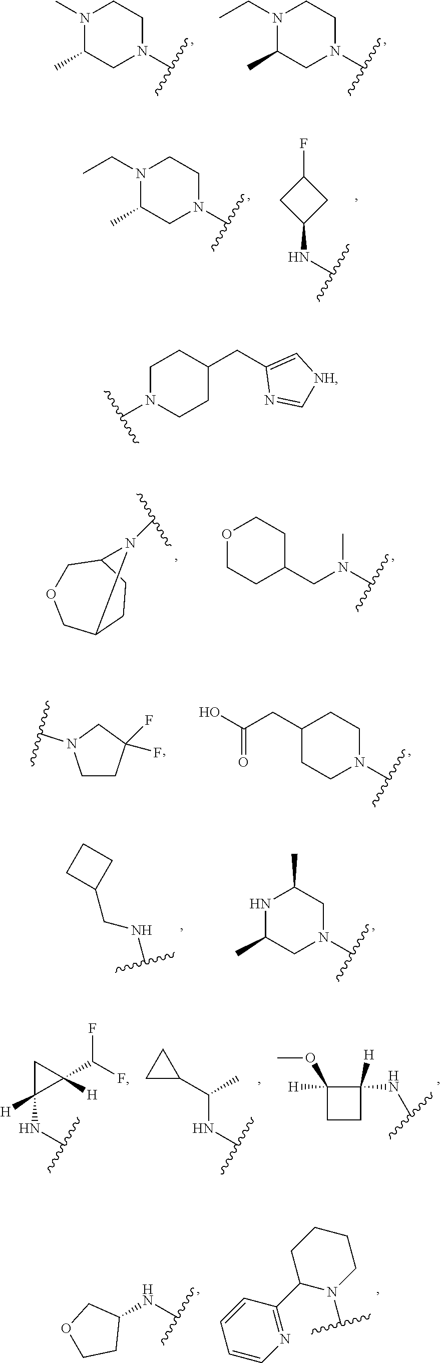

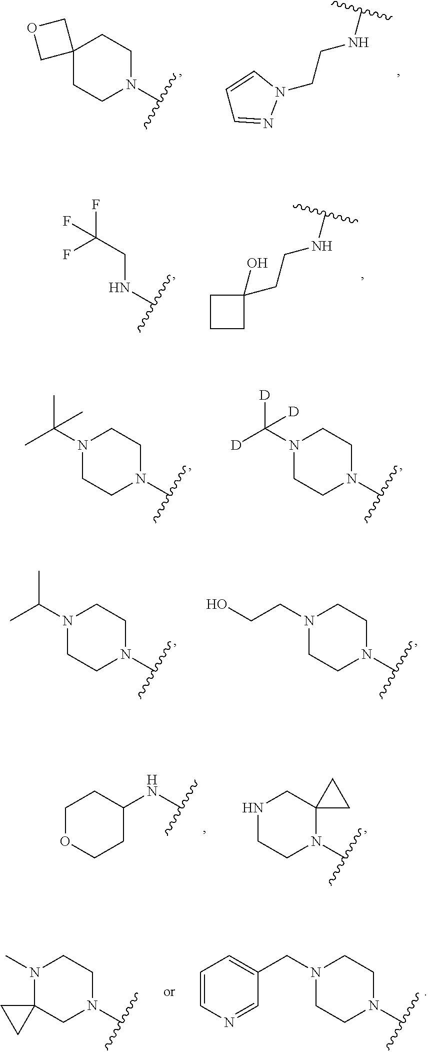

B-pocket substituent is selected from the group consisting of

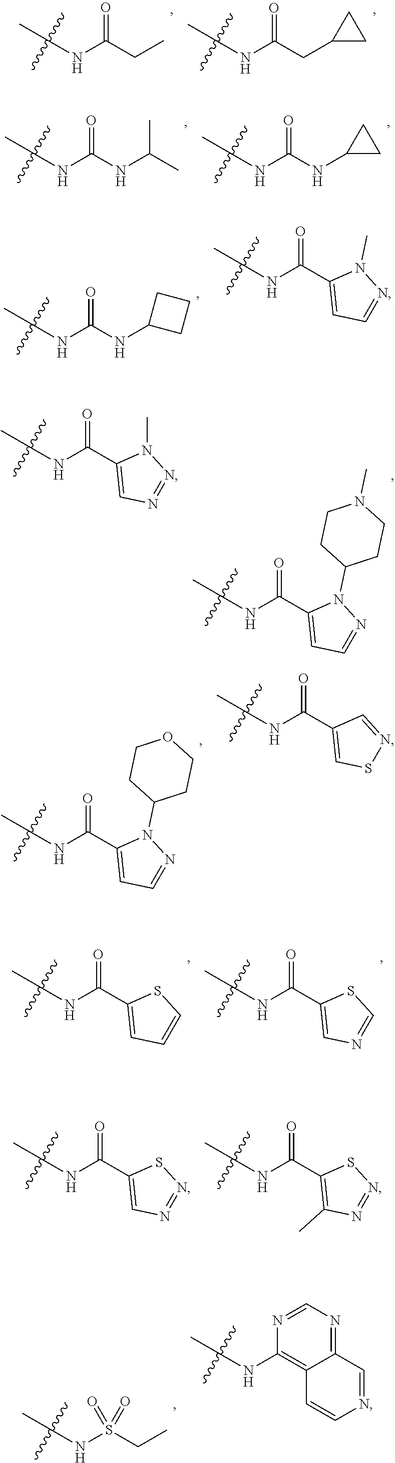

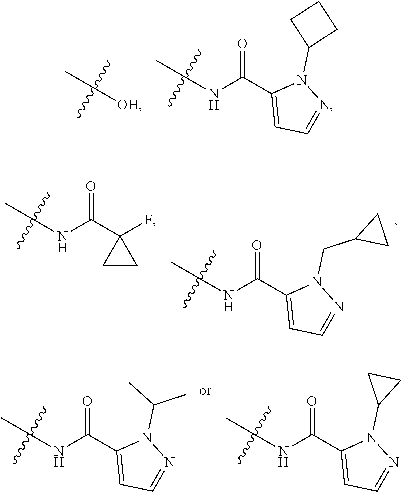

D-pocket substituent is selected from the group consisting of

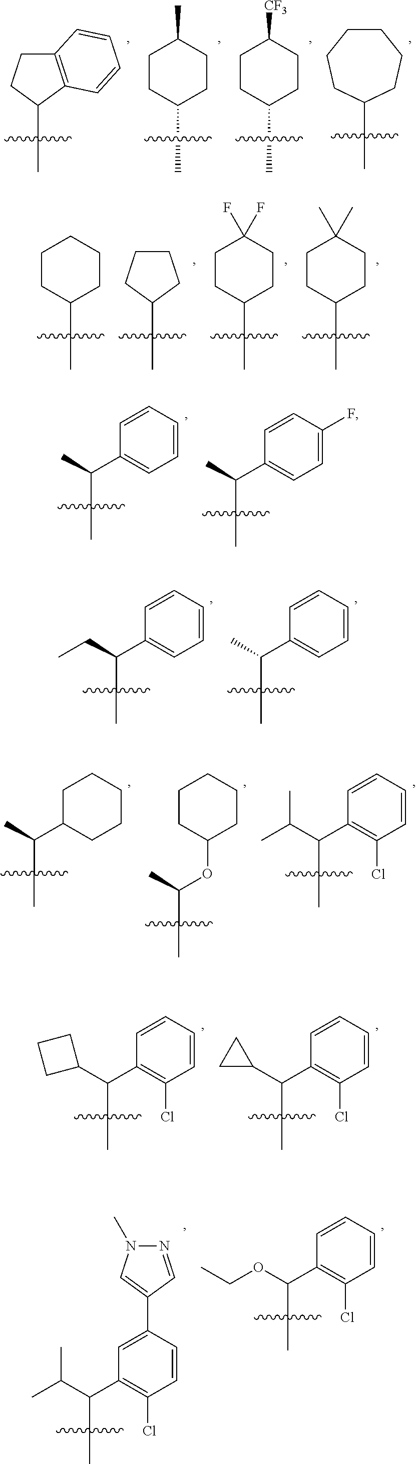

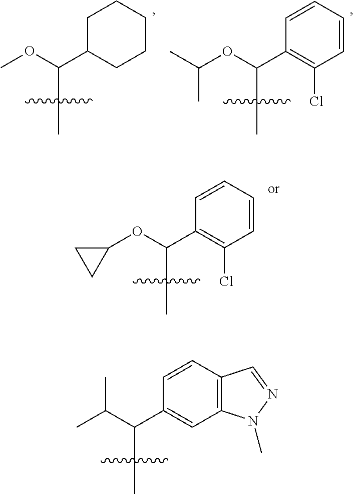

F-pocket substituent is selected from the group consisting of

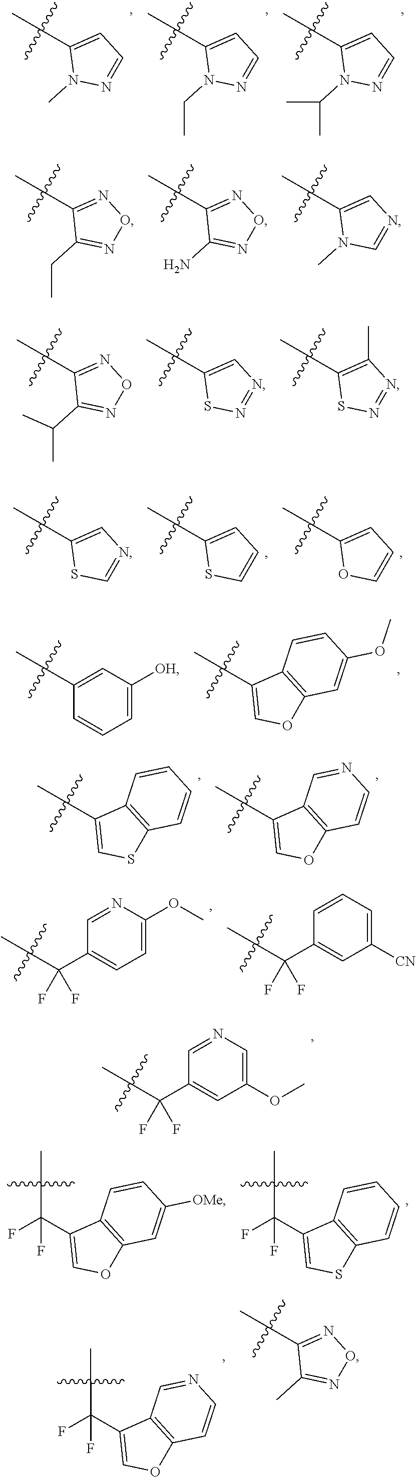

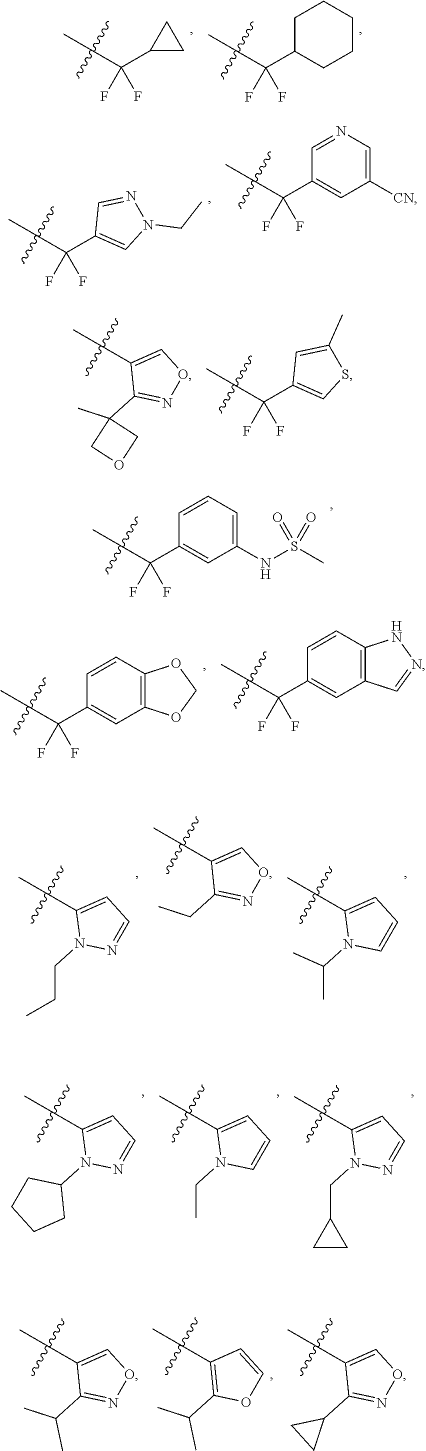

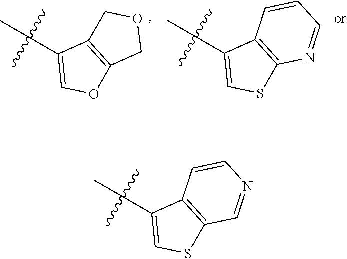 [SMILES: *c1coc2c1COC2 ; *c1csc2ncccc12 ; *c1csc2cnccc12]

wherein each optional substituent for the above Formula is independently selected from halogen, —OR^{F12}, —SR^{F12}, —N(R^{F12})₂, —C(O)R^{F12}, —C(O)N(R^{F12})₂, N(R^{F12})C(O)R^{F12}, —C(O)OR^{F12}, —OC(O)R^{F12}, —S(O)R^{F12}, —S(O)₂R^{F12}, —NO₂, ═O, ═S, ═N(R^{F12}), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR^{F12}, —N(R^{F12})₂, —C(O)R^{F12}, —C(O)N(R^{F12})₂, —N(R^{F12})C(O)R^{F12}, —C(O)OR^{F12}, —OC(O)R^{F12}, —NO, ═O, —N(R^{F11}) and —CN.

In certain embodiments the I-17 Targeting Ligand is of Formula:

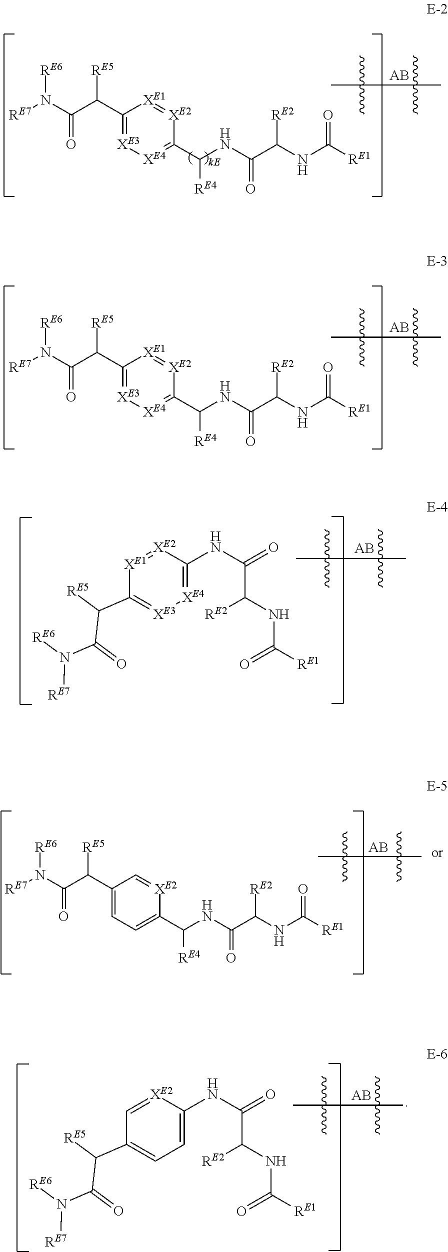

In certain embodiments IL-17 Targeting Ligand is of Formula:

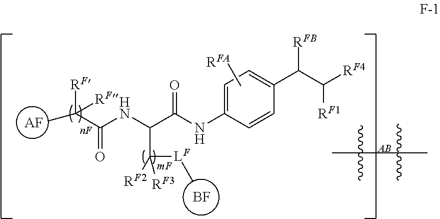

wherein,

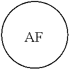

is selected from an optionally substituted C₃₋₁₂ carbocycle and optionally substituted 3- to 12-membered heterocycle wherein substituents on Ring AF are independently selected at each occurrence from:
halogen, —OR^{F11}, —SR^{F11}, —N(R^{F11})₂, —C(O)R^{F11}, —C(O)N(R^{F11})₂, N(R^{F11})C(O)R^{F11}, —N(R^{F11}) S(O)₂R^{F11}, —C(O)OR^{F11}, —OC(O)R^{F11}, —S(O)R^{F11}, —S(O)₂R^{F11}, —NO₂, ═O, ═S, ═N(R^{F11}), —CN; and
C₁₋₁₀ alkyl, C₂₋₁₀ alkenyl, C₂₋₁₀ alkynyl, each of which is optionally substituted with one or more substituents independently selected from halogen, —OR^{F11}, —SR^{F11}, —N(R^{F11})₂, —C(O)R^{F11}, —C(O)N(R^{F11})₂, · N(R^{F11})C(O)R^{F11}, —C(O)OR^{F11}, —OC(O)R^{F11}, —S(O)R^{F11}, —S(O)₂R^{F11}, —NO₂, ═O, ═S, ═N(R^{F11}), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR^{F11}, —N(R^{F11})₂, —C(O)R^{F11}, —C(O)N(R^{F11})₂, —N(R^{F11})C(O)R^{F11}, —C(O)OR^{E1}, —OC(O)R^{E1}, —NO 2, =O═N(R^{F11} and —CN· ′and
C₃₋₁₂ carbocycle and 3- to 12-membered heterocycle, each of which is optionally substituted with one or more substituents independently selected from: halogen, —OR^{F11}, —SR^{F11}, —N(R^{F11})₂, —C(O)R^{F11}, —C(O)N(R^{F11})₂, N(R^{F11})C(O)R^{F11}, —C(O)OR^{F11}, —OC(O)R^{F11}, —NO₂, —CN, C₁₋₆ alkyl and C₁₋₆ haloalkyl;

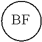

is selected from an optionally substituted C₃₋₁₀ carbocycle and optionally substituted 3- to 12-membered heterocycle each substituent on Ring B are independently selected at each occurrence from:
halogen, —OR^{F12}, —SR^{F12}, —N(R^{F12})₂, —C(O)R^{F12}, —C(O)N(R^{F12})₂, —N(R^{F12})C(O)R^{F12}, —C(O)OR^{F12}, —OC(O)R^{F12}, —S(O)R^{F12}, —S(0)₂R^{F12}, —N02, —O, ═S, ═N(R^{F12}), —CN; and
C₁₋₁₀ alkyl, C₂₋₁₀ alkenyl, C₂₋₁₀ alkynyl, each of which is optionally substituted with one or more substituents independently selected from halogen, —OR^{F12}, —SR^{F12}, —N(R^{F12})₂, —C(O)R^{F12}, —C(O)N(R^{F12})₂, N(R^{F12})C(O)R^{F12}, —C(O)OR^{F12}, —OC(O)R^{F12}, —S(O)R^{F12}, —S(O)₂R^{F12}, —NO₂, ═O, ═S, ═N(R^{F12}), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR^{F12}, —N(R^{F12})₂, —C(O)R^{F12}, —C(O)N(R^{F12})₂, —N(R^{F12})C(O)R^{F12}, —C(O)OR^{F12}, —OC(O)R^{F12}, —NO, —O, ═N(R^{F11}) and —CN;
R^{F4} is selected from—C(O)N(R^{F23}) (R^{F24}) and C(O) heterocycle, wherein heterocycle is optionally substituted with 1, 2, 3, or 4 substituents selected from halogen, —OR^{F13}, —SR^{F13}, —N(R^{F13})₂, —C(O)R^{F13}, —C(O)N(R^{F13})₂, —N(R^{F13})C(O)R^{F13}, —C(O)OR^{F13}, —OC(O)R^{F13}, —S(O)R^{F13}, —S(O)₂R^{F13}, —NO₂, ═O, ═S, ═N(R^{F13})—CN; and
C₁₋₁₀ alkyl, C₂₋₁₀ alkenyl, C₂₋₁₀ alkynyl, each of which is optionally substituted with one or more substituents independently selected from halogen, —OR^{F13}, —SR^{F13}, —N(R^{F13})₂, —C(O)R^{F13}, —C(O)N(R^{F13})₂, N(R^{F13})C(O)R^{F13}, —C(O)OR^{F13}, —OC(O)R^{F13}, —S(O)R^{F13}, —S(O)₂R^{F13}, —NO₂, ═O, ═S, ═N(R^{F13}), —CN, C₃₋₁₀ carbocycle and 3- to 10-membered heterocycle, wherein the C₃₋₁₀ carbocycle and 3- to 10-membered heterocycle are each optionally substituted with one or more substituents selected from: halogen, —OR^{F13}, —N(R^{F13})₂, —C(O)R^{F13}, —C(O)N(R^{F13})₂, —N(R^{F13})C(O)R^{F13}, —C(O)OR^{F13}, —OC(O)R^{F13}, —NO₂, —O, ═N(R^{F13}), and —CN;
L^{F} is bond or selected from —O- and —NH—;
R^{FA} is selected from hydrogen, halogen, —OR^{F14}, —N(R^{F14})₂, —C(O)R^{F14}, —C(O)N(R^{F14})₂, N(R^{F14})C(O)R^{F14}, —C(O)OR^{F14}, —OC(O)R^{F14}, —NO₂, —CN, and C₁₋₆ alkyl, wherein C₁₋₆ alkyl is optionally substituted with one or more substituents selected from: halogen, OR^{F14}, —N(R^{F14})₂, —C(O)R^{F14}, NO₂, ═O, and —CN;
R^{FB} is selected from hydrogen, halogen, —OR^{F15}, —N(R^{F15})₂, —C(O)R^{F15}, —C(O)N(R^{F15})₂, N(R^{F15})C(O)R^{F15}, —C(O)OR^{F15}, —OC(O)R^{F15}, —NO₂, —CN, and C₁₋₆ alkyl, wherein C₁₋₆ alkyl is optionally substituted with one or more substituents selected from: halogen, OR^{F15}, —N(R^{F15})₂, —C(O)R^{F15}, NO₂, ═O, and —CN, wherein at least one of RA or RB is not hydrogen;
R^{F′} and R^{F″} are independently selected from: hydrogen, halogen, —OR^{F16}, and C₁₋₆ alkyl; wherein the C₁₋₆ alkyl is optionally substituted with one or more substituents selected from: halogen, —OR^{F16}, —N(R^{F16})₂, —C(O)R^{F16}, —NO₂, ═O, and —CN;
R^{F1} is selected from —OR^{F21}, —N(R^{F21}) (R^{F22}), —N(R^{F21})C(O)R^{F22}, —N(R^{F21})C(O)OR^{F22}, —N(R^{F21})C(O)N(R^{F21}) (R^{F22}), —N(R^{F21}) S(═O)₂N(R^{F21}) (R^{F22}), and —N(R^{F21}) S(═O)₂ (R^{F22});
each R^{F2} and R^{F3} are independently selected from: hydrogen, halogen, —OR^{F17}, C₁₋₆ alkyl, and C₃₋₆ cycloalkyl; wherein the C₁₋₆ alkyl and C₃₋₆ cycloalkyl are optionally substituted with one or more substituents selected from: halogen, —OR^{F17}, —N(R^{F17})₂, —C(O)R^{F17}, —NO₂, —O, and —CN; or R^{F2} and R^{F3} bound to the same carbon come together to form a C₃₋₆ cycloalkyl optionally substituted with one or more substituents selected from halogen, —OR^{F17}, —N(R^{F17})₂, —C(O)R^{F17}, —NO₂, —O, and —CN;
R^{F21} is independently selected at each occurrence from hydrogen and C₁-C₆ alkyl optionally substituted by one or more substituents independently selected from halogen, —OR^{F17}, —N(R^{F17})₂, —C(O)R^{F17}, —NO₂, —O, and —CN;
R^{F22} is selected from: C₁₋₁₀ alkyl, C₂₋₁₀ alkenyl, C₂₋₁₀ alkynyl, each of which is optionally substituted with one or more substituents independently selected from halogen, —OR^{F18}, —SR^{F18}, —N(R^{F18})₂, —C(O)R^{F18}, —C(O)N(R^{F18})₂, —N(R^{F18})C(O)R^{F18}, —C(O)OR^{F18}, —OC(O)R^{F18}, —S(O)R^{F18}, —S(O)₂R^{F18}, —NO₂, —O, ═S, ═N(R^{F18}), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR^{F18}, —N(R^{F18})₂, —C(O)R^{F18}, —C(O)N(R^{F18})₂, ·N(R^{F18})C(O)R^{F18}, —C(O)OR^{F18}, —OC(O)R^{F18}, —NO₂, ═O, ═N(R^{F18}), and —CN; and C₃₋₁₂ carbocycle and 3- to 12-membered heterocycle, each of which is optionally substituted with one or more substituents independently selected from:
halogen, —OR^{F18}, —SR^{F18}, —N(R^{F18})₂, —C(O)R^{F18}, —C(O)N(R^{F18})₂, —N(R^{F18})C(O)R^{F18}, —C(O)OR^{F18}, —OC(O)R^{F18}, —S(O)R^{F18}, —S(O)₂R^{F18}, —NO₂, ═O, ═S, ═N(R^{F18}), —CN; and
C₁₋₁₀ alkyl, C₂₋₁₀ alkenyl, C₂₋₁₀ alkynyl, each of which is optionally substituted with one or more substituents independently selected from halogen, —OR^{F18}, —SR^{F18}, —N(R^{F18})₂, —C(O)R^{F18}, —C(O)N(R^{F18})₂, —N(R^{F18})C(O)R^{F18}, —C(O)OR^{F18}, —OC(O)R^{F18}, —S(O)R^{F18}, —S(O)₂R^{F18}, —NO₂, ═O, ═S, ═N(R^{F18}), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR^{F18}, —N(R^{F18})₂, —C(O)R^{F18}, —C(O)N(R^{F18})₂, —N(R^{F18})C(O)R^{F18}, —C(O)OR^{F18}, —OC(O)R^{F18}, —NO═O, ═N(R^{F18}), and —CN; and
C₃₋₁₀ carbocycle and 3- to 10-membered heterocycle are each optionally substituted with one or more substituents selected from: halogen, —OR^{F18}, —N(R^{F18})₂, —C(O)R^{F18}, —C(O)N(R^{F18})₂, N(R^{F18})C(O)R^{F18}, —C(O)OR^{F18}, —OC(O)R^{F18}, —NO₂, ═O, ═N(R^{F18}), and —CN; R^{F23} is selected from:
C₁₋₆ alkyl optionally substituted with one or more substituents independently selected from halogen, —OR^{F19}, —SR^{F19}, —N(R^{F19})₂, —NO₂, —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR^{F19}, —N(R^{F19})₂, —O, C₁-C₆ alkyl, C₁-C₆ haloalkyl, and —CN; and
C₃₋₁₂ carbocycle and 3- to 10-membered heterocycle, each of which is optionally substituted with one or more substituents independently selected from halogen, —OR^{F19}, —N(R^{F19})₂, ═O, C₁-C₆ alkyl, C₁-C₆ haloalkyl, and —CN;
R^{F24} is selected from hydrogen and C₁₋₆ alkyl optionally substituted with one or more substituents independently selected from halogen, —OR^{F19}, —SR^{F19}, —N(R^{F19})₂, —NO₂, —CN, C₃₋₆ carbocycle and 3- to 6-membered heterocycle;
R^{F11}, R^{F12}, R^{F13}, R^{F14}, R^{F15}, R^{F16}, R^{F17}, R^{F18}, and R^{F19} are independently selected at each occurrence from
hydrogen; and
C₁₋₆ alkyl optionally substituted with one or more substituents independently selected from halogen, —OH, —O—C₁-C₆ alkyl, —O—C₁-C₆haloalkyl-NH₂, —NO ₂, ═O, —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OH, —O—C₁-C₆ alkyl, —O—C₁-C₆ haloalkyl-NH₂, —NO₂, —O, and —CN; and
C₃₋₁₂ carbocycle and 3- to 12-membered heterocycle, each of which is optionally substituted with one or more substituents independently selected from:
halogen, —OH, —O—C₁-C₆ alkyl, —O—C₁-C₆haloalkyl-NH₂, —NO₂, —O, —CN; and C₁₋₆ alkyl optionally substituted with one or more substituents independently selected from halogen, —OH, —O—C₁-C₆ alkyl, —O—C₁-C₆ haloalkyl-NH₂, —NO₂, —O, and —CN;
n^{F} is selected from 0 and 1; and
m^{F} is selected from 0, 1, and 2.

In certain embodiments IL-17 Targeting Ligand is of Formula:

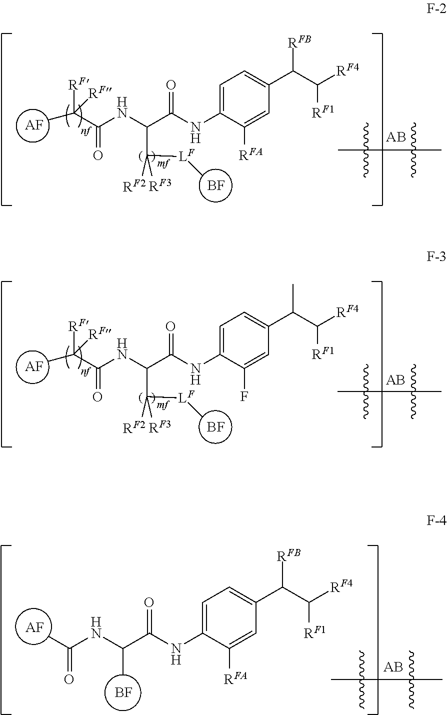

In certain embodiments IL-17 Targeting Ligand is of Formula:

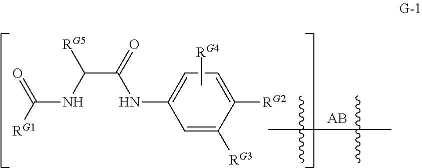

wherein;

R^{G1} is selected from the group consisting of 5- or 6-membered heteroaryl, 9- or 10-membered bicyclic heteroaryl, phenyl, (C₁-C₆)alkoxy, (C₃-C₇)cycloalkoxy, (C₁-C₆)alkyl, phenyl-(C₁-C₄)alkyl, (C₃-C₇)cycloalkyl, 4-6-membered heterocycloalkyl and —NR^{GC}R^{GD}, wherein said 5- or 6-membered heteroaryl, 9- or 10-membered bicyclic heteroaryl, phenyl, (C₁-C₆)alkoxy, (C₃-C₇)cycloalkoxy, (C₁-C₆)alkyl, phenyl-(C₁-C₄)alkyl, (C₃-C₇)cycloalkyl and 4-6-membered heterocycloalkyl is optionally substituted with one or more substituents independently selected from R^{GA};
R^{GA} represents deuterium, halogen, hydroxy, —NR^{GC}R^{GD}, (C₁-C₆)alkyl, (C₁-C₆)alkylcarbonyl, (C₃-C₇)cycloalkyl, phenyl, 5- or 6-membered heteroaryl or, 4-6-membered heterocycloalkyl, wherein said (C₁-C₆)alkyl, (C₁-C₆)alkylcarbonyl, (C₃-C₇)cycloalkyl, phenyl, 5- or 6-membered heteroaryl or 4-6-membered heterocycloalkyl is optionally substituted with one or more substituents independently selected from deuterium, halogen, hydroxy, cyano, (C₁-C₄)alkyl, (C₃-C₇)cycloalkyl, (C₁-C₄)alkoxy, —SO2—(C₁-C₄)alkyl and —NR^{GC}R^{GD};
R^{G2} is selected from the group consisting of 5- or 6-membered heteroaryl, wherein said 5- or 6-membered heteroaryl is optionally substituted with one or more substituents independently selected from RGB, wherein said 5- or 6-membered heteroaryl may optionally contain —CO—as a ring member and wherein when said 5 membered heteroaryl contains nitrogen as a ring atom said nitrogen may optionally be substituted with a substituent selected from R^{G8};
R^{GB} represents deuterium, halogen, cyano, hydroxy, —NR^{GC}R^{GD}, (C₁-C₆)alkyl, (C₁-C₆)alkoxy, (C₁-C₆)alkyl-CO—O—(CH₂)ₙ—or (C₃-C₇)cycloalkyl, wherein n is 1-4, and wherein said (C₁-C₆)alkyl, (C₁-C₆)alkoxy or (C₃-C₇)cycloalkyl is optionally substituted with one or more substituents independently selected from deuterium, halogen, cyano, hydroxy, —NR^{GC}R^{GD} and (C₁-C₄)alkoxy;
R^{GC} and R^{GD} each independently are selected from the group consisting of hydrogen and (C₁-C₆)alkyl, or R^{GC} and R^{GD} together form pyrrolidinyl or piperidinyl, wherein said (C₁-C₆)alkyl, pyrrolidinyl or piperidinyl is optionally substituted with one or more substituents independently selected from halogen, cyano and hydroxy;
R^{G8} is selected from the group consisting of —L^{G}-PO(OH)₂ and —CHR^{GG}O—(CO-A-NR^{GH}))_{0 or 1)}—CO-A-NR^{GH}R^{GI};

LG is selected from the group consisting of a bond or —CHR^{GG}O—;
wherein each —CO-A-NR^{GH}—independently represents an amino acid residue wherein the amino acid residue is selected from the natural amino acids either in D or L-form or as mixtures of the D and L form, and wherein said amino acid residue may be substituted on the α-amino group with a substituent R^{GH};
R^{GG}, R^{GH}, and R^{GI} are independently selected from hydrogen and (C₁-C₆)alkyl;
R^{G3} is selected from the group consisting of hydrogen, deuterium, hydroxy and halogen;
R^{G4} is selected from the group consisting of hydrogen, deuterium and halogen;
R^{G5} is selected from the group consisting of —CHR^{G6}R^{G7}, (C₃-C₁₀)cycloalkyl and GG, wherein said (C₃-C₁₀)cycloalkyl and GG are optionally substituted with one or more substituents independently selected from deuterium, halogen, cyano, hydroxy, (C₁-C₄)alkyl and halo(C₁-C₄)alkyl;
GG represents

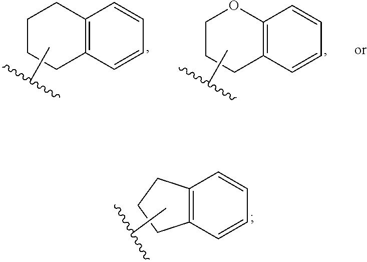

and
R^{G6} and R^{G7} each independently represents hydrogen, phenyl, (C₁-C₆)alkyl, or (C₃-C₇)cycloalkyl, wherein said phenyl, (C₁-C₆)alkyl or (C₃-C₇)cycloalkyl is optionally substituted with one or more substituents independently selected from halogen, cyano, hydroxy and (C₁-C₄)alkyl.

In certain embodiments IL-17 Targeting Ligand is of Formula:

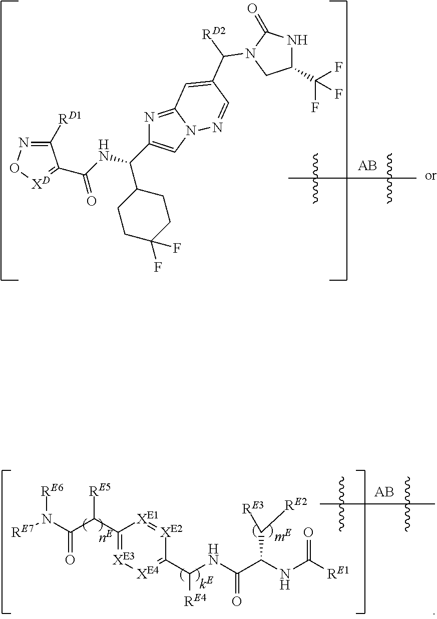

### Interleukin-6 (IL-6)

In some embodiments, the Target Extracellular Protein is human inteleukin-6 (IL-6) (UniProtKB-P05231 (IL6_HUMAN)). IL-6 is a cytokine with a wide variety of biological functions. It is a potent inducer of the acute phase response and plays an essential role in the final differentiation of B-cells into Ig-secreting cells. It is also involved in lymphocyte and monocyte differentiation. It also acts on B-cells, T-cells, hepatocytes, hematopoietic progenitor cells and cells of the CNS, and is required for the generation of T (H) 17 cells. IL-6 has been implicated in a number of inflammatory diseases and cancers, including, but not limited to, Castleman's disease, metastatic castration- associated prostate cancer, renal cell carcinoma, large-cell lung carcinoma, ovarian cancer, rheumatoid arthritis, asthma.

The Protein Data Bank website provides the crystal structure of IL-6 searchable by 1P9M (Boulanger, M. J., et al., Science, 2003, 300:2101-2104); 1ALU (Somers et al., EMBO J., 1997, 16, 989-997); 1IL6 and 2IL6 (Xu, G. Y., et al., J Mol Biol., 1997, 268 468-481) and 1N26 (Varghese et al., Proc Natl Acad Sci USA., 2002, 99 15959-15964); as well as the crystal structure of IL-6 bound to various compounds searchable by 4CNI (Shaw, S., et al., Mabs, 2014, 6:773); and 4NI7 and 4NI9 (Gelinas et al., J Biol Chem. 2014, 289 (12), 8720-8734). Additionally, Gelinas et al., provides insight into the crystal structure of interleukin-6 in complex with a modified nucleic acid ligand (Gelinas, A. D., et al., J Biol Chem. 2014, 289 (12), 8720-8734); and Somers et al., provides insight into the crystal structure of interleukin 6: implications for a novel mode of receptor dimerization and signaling.

Non-limiting examples of IL-6 direct or indirect inhibitors are provided in FIG. 1. Additional IL-6 direct or indirect inhibitors can be found in, for example, U.S. Pat. Nos. 8,901,310; 10,189,796; 9,694,015; each incorporated herein by reference. In another embodiment the IL-6 Extracellular Targeting Ligand is AvimarC326 or a binding fragment thereof which is described in Nat Biotechnol 23, 1556-1561 (2005).

In some embodiments, the Target Extracellular Protein is Interleukin-6. Interleukin-6 (IL-6) is a cytokine that is a crucial component of the acute phase immune response. IL-6 ligand binds to IL-6 receptor, and the heterodimer then associates with IL6ST and gp130, stimulating a response. During infection certain molecules from pathogens bind to toll-like receptors, which activate macrophages to produce IL-6. In addition to stimulating differentiation of B cells and neutrophils, IL-6 mediates the fever response.

IL-6 has been implicated in many inflammatory diseases, including multiple sclerodid, neuromyelitis optica spectrum disorder, diabetes, atherosclerosis, depression, Alzheimer's disease, systemic lupus erythromatosus, multiple myeloma, prostate cancer, Behcet's disease, rheumatoid arthritis, systemic juvenile idiopathic arthritis, and Castleman's disease.

IL-6 signaling is also important to the musculoskeletal system. In bone, it interacts with VEGF stimulating angiogenesis. In muscle cells, IL-6 is produced in large amounts during exercise. In contrast to its role in stimulating the immune system, during exercise IL-6 is anti-inflammatory.

The Protein Data Bank website provides the crystal structure of Interleukin-6, searchable by 1ALU (Somers, W. S. et al. 1.9 A crystal structure of interleukin 6: implications for a novel mode of receptor dimerization and signaling. (1997) EMBO J. 16:989-997), 1IL6 (Xu, G. Y. et al. Solution structure of recombinant human interleukin-6 (1997) J Mol Biol 268:468-481), and the structure of IL-6 bound in the active hexameric complex searchable by 1P9M (Boulanger, M. J. et al. Hexameric Structure and Assembly of the Interleukin-6/IL-6-alpha-Receptor/gp130 Complex. (2003) Science 300:2101-2104)

Non-limiting examples of IL-6 Targeting Ligands can be found in, for example, USP 10633423, USP 10669314, US 2004/0092720, and Ranganath, S. et al. Discovery and Characterization of a Potent Interleukin-6 Binding Peptide with Neutralizing Activity In Vivo.

PLOS ONE 10(11): e0141330.
SEQ ID NO: 388
QSDChaDCIHRLLEAF(4-F)LDPNLTEEQRWEKIGlaKINDECE
(Ranganath, S. et al. PLOS ONE 10(11): e0141330)
SEQ ID NO: 389
QSDChaDCIHRLLEAF(4-F)LDPNLTEEQRWERIGlaK(PEG30L)INDECE
(Ranganath, S. et al. PLOS ONE 10(11): e0141330)
SEQ ID NO: 390
QSDChaDCIHRLLEAF(4-F)LDPNLTEEQRWERIGlaK(PEG20Br)INDECE
(Ranganath, S. et al. PLOS ONE 10(11): e0141330)
SEQ ID NO: 391
QSDChaDCIHRLLEAF(4-F)LDPNLTEEQRWERIGlaK(PEG40Br)INDECE
(Ranganath, S. et al. PLOS ONE 10(11): e0141330)
SEQ ID NO: 392
FDhLDCIHRLLEAFLDPNLTEQQRWEKIDKINDECE (Ranganath, S. et
al. PLOS ONE 10(11): e0141330)
SEQ ID NO: 393
QSDChaDCIHRLLEAF(4-F)LDPNLTEEQRWERIGlaKINDECE
(Ranganath, S. et al. PLOS ONE 10(11): e0141330)
SEQ ID NO: 394
SWQSDChaDCIHRLLEAFLDK-AcNLTEEQRWERIDKINDECE
(Ranganath, S. et al. PLOS ONE 10(11): e0141330)
SEQ ID NO: 395
SWQSDChaDCIHRLLEAFLDK-(PEG40Br)-
NLTEEQRWERIDKINDECE (Ranganath, S. et al. PLOS ONE 10(11): e0141330)

In certain embodiments the IL-6 Targeting ligand is SEQ ID NO: 388, bound to the linker through the PEGylated lysine residue.

SEQ ID NO: 396
EEX₃X₄AWX₇EIHX₁₁LPNLX₁₆X₁₇X₁₈QX₂₀X₂₁AFIX₂₅X₂₆LX₂₈X₂₉
(U.S. Pat. No. 10, 633,423)

wherein, independently from each other;
X₃ is selected from A, F, H, K, Q, R, S, W and Y;
X₄ is selected from A, D, E, F, H, I, K, L, M, N, Q, R, S, T, V and Y;
X₇ is selected from F, H, I, K, L, M, N, R, S, T, V, W and Y;
X₁₁ is selected from A, I, K, L, M, N, R, S, T and V;
X₁₆ is selected from N and T;
X₁₇ is selected from A, I, T and V;
X₁₈ is selected from D, E, G, H, K, N, Q, R, S and T;
X₂₀ is selected from I, L, M, R, T and V;
X₂₁ is selected from A, S, T and V;
X₂₆ is selected from I, M, Q, S, T, V and W;
X₂₆ is selected from K and S,
X₂₈ is selected from F, L, M and Y; and
X₂₉ is selected from D and R;

SEQ ID NO: 397
EEX₃X₄AWX₇EIHX₁₁LPNLX₁₆X₁₇X₁₈QX₂₀X₂₁AFIX₂₅X₂₆LX₂₈X₂₉
(U.S. Pat. No. 10, 669,314)

wherein, independently from each other,
X₃ is selected from A, F, H, K, Q, R, S, W and Y;
X₄ is selected from A, D, E, F, H, I, K, L, M, N, Q, R, S, T, V and Y;
X₇ is selected from F, H, I, K, L, M, N, R, S, T, V, W and Y;
X₁₁ is selected from A, I, K, L, M, N, R, S, T and V;
X₁₆ is selected from N and T;
X₁₇ is selected from A, I, T and V;
X₁₈ is selected from D, E, G, H, K, N, Q, R, S and T;
X₂₀ is selected from I, L, M, R, T and V;
X₂₁ is selected from A, S, T and V;
X₂₅ is selected from I, M, Q, S, T, V and W;
X₂₆ is selected from K and S;
X₂₈ is selected from F, L, M and Y; and
X₂₉ is selected from D and R;

In certain embodiments the targeting ligand for treating an IL-6 mediated disease binds to gp130. Non-limiting examples of gp130 Targeting Ligands can be found in, for example, Ahn, S—H. et al. In vitro and in vivo pharmacokinetic characterization of LMT-28 as a novel small molecular interleukin-6 inhibitor 2020 Asian-Australas J Anim Sci.33:670-677, Aqel, S. I. Novel small molecule IL-6 inhibitor suppresses autoreactive Th17 development and promotes Treg development. (2019)Clinical and Experimental Immunology, 196:215-225, Hong, S.-S. et al. A Novel Small-Molecule Inhibitor Targeting the IL-6 Receptor beta Subunit, Glycoprotein 130. 2015 J Immunol 195:237-245

Non-limiting examples of gp130 Targeting Ligands can be found in, for example, Wang, J. et al. “Structure-based virtual screening and characterization of a novel IL-6 antagonistic compound from synthetic compound database” Drug Design, Development and Therapy 2016:10 4091-4100

In certain embodiments the gp130 binding Targeting Ligand is selected from

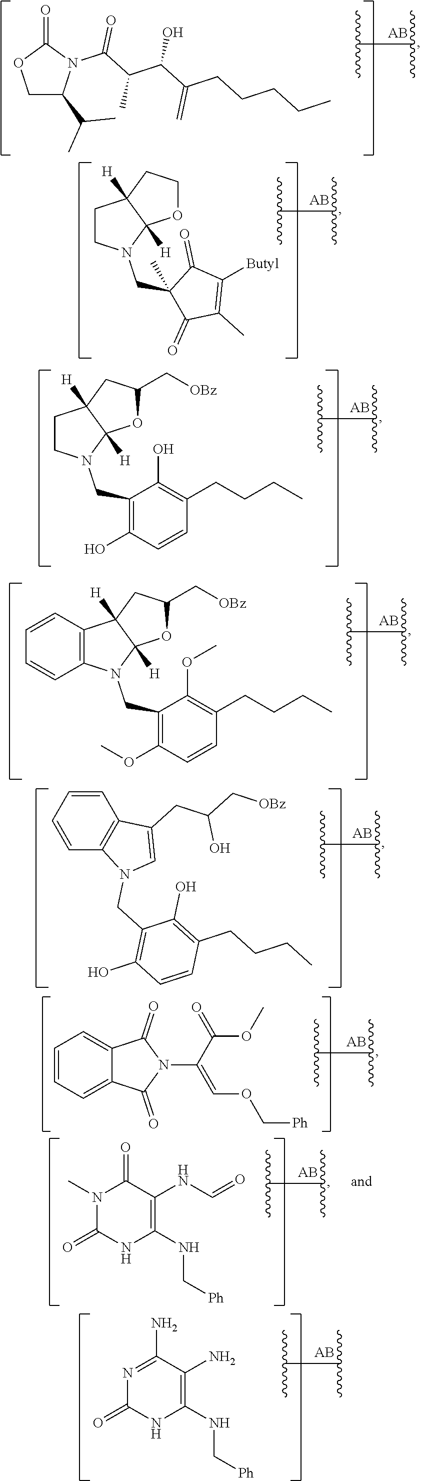

### Immunoglobulin A1 (IgA1)

Immunoglobulin A is a class of antibodies which is commonly found in secretions, but is also present in serum. IgA contains four heavy chains and four light chains, in a dimeric form. IgA exists in two isotypes, IgA1 and IgA2. IgA1 contains more repeats in the hinge region and is the predominant form found in serum. While production of IgA maintains strong mucosal immunity and defending against pathogens, it can become toxic. IgA nephropathy, also known as Berger's disease, is the pathological buildup of IgA antibodies which reduces kidney function. The etiology of the disease remains unclear, however it has been suggested that the glycosylation pattern on the hinge region plays a role. As proper kidney function is important for overall health, IgA nephropathy is associated with systemic diseases such as liver failure, cancer, celiac disease, systemic lupus erythematosus, rheumatoid arthritis, heart failure, reactive arthritis, and ankylosing spondylitis.

The Protein Data Bank website provides the crystal structure of IgA1, and representative example include PDB accession codes 1IGA (Boehm, M. K. 1999, J. Mol. Bio. 286 1421-1447), 2ESG (Almogren, A. 2006 J. Mol. Biol. 356, 413-431), 6XJA, 7JGJ, (E isenmesser, E. Z. 2020, Nat. Commun, 11, 6063-6063), and 3CHN (Bonner, A. 2009, Mucosal Immunol., 2, 74-84).

Direct or indirect IgA1 binding molecules include jacalin and

SEQ ID NO: 398
YYALSDAKEEEPRYKALRGENQDLREKERKYQDKIKKLEEKEKNLEKKS.

In certain embodiments, the IgA1 Targeting Ligand is selected from a ligand described in US20090317381A1; US20210301019A1; U.S. Pat. Nos. 4,757,134; 5,210,183; 5,644,030; 5,714,334; 5,723,303; 5,869,047; Ramsland, P. et al. “Structural basis for evasion of IgA immunity by Staphylococcus aureus revealed in the complex of SSL7 with Fc of human IgA1” PNAS 2007, 104:38, 15051-15056; and Herr, A. et al. “Insights into IgA-mediated immune responses from the crystal structures of human FcaRI and its complex with IgA1-Fc” Nature 2003, 423, 614-620.

### Anti-B1AR Autoantibody Targeting Ligand

In certain aspects a Extracellular Protein Degrading Compound of the present invention degrades anti-β1 adrenergic receptor (anti-β1AR) autoantibodies and can be used to treat a disorder mediated by anti-β1AR autoantibodies such as heart failure, for example cardiomyopathy or dilated cardiomyopathy.

In certain embodiments the anti-B1AR autoantibody Targeting Ligand is SEQ ID NO: 399 DEARRCYNDPKCSDFVQ. In certain embodiments, the anti-β1AR autoantibody targeting ligand has about 98%, 95%, 93%, 90%, 88%, 85%, 83%, or 80% sequence homology with SEQ ID NO: 399. In certain embodiments, the anti-β1AR autoantibody Targeting Ligand is SEQ ID NO: 399, cyclized from N-terminus to C-terminus. In certain embodiments, the anti-β1AR autoantibody Targeting Ligand is SEQ ID NO: 399, with a disulfide bond between the cysteine residues. In certain embodiments, the anti-β1AR autoantibody Targeting Ligand is SEQ ID NO: 399, cyclized from N-terminus to C-terminus and with a disulfide bond between the cysteine residues.

In certain embodiments the anti-β1AR autoantibody Targeting Ligand is SEQ ID NO: 420 ADEARRCYNDPKCSDFVQ. In certain embodiments, the anti-β1AR autoantibody targeting ligand has about 98%, 95%, 93%, 90%, 88%, 85%, 83%, or 80% sequence homology with SEQ ID NO: 420. In certain embodiments, the anti-β1AR autoantibody Targeting Ligand is SEQ ID NO: 420, cyclized from N-terminus to C-terminus. In certain embodiments, the anti-β1AR autoantibody Targeting Ligand is SEQ ID NO: 399, with a disulfide bond between the cysteine residues.

### Prostate Specific Membrane Antigen (PSMA)

In some embodiments, the Target Extracellular Protein is human prostate specific membrane antigen (UniProtKB-Q04609 (FOLH1_HUMAN)), also known as Glutamate carboxypeptidase II (GCPII), N-acetyl-L- aspartyl-L-glutamate peptidase I (NAALADase I), and NAAG peptidase. PSMA is an enzyme that catalyzes the reaction of N-Acetyl aspartylglutamate to glutamate and N-acetylaspartate. PSMA inhibitors have been shown to decrease the levels of glutamate in the nervous system, protecting from neural degeneration in models of stroke, amyotrophic lateral sclerosis (ALS) and neuropathic pain. In certain embodiments, the Extracellular Protein Targeting Ligand binds to PSMA.

Nonlimiting examples of PSMA ligands include

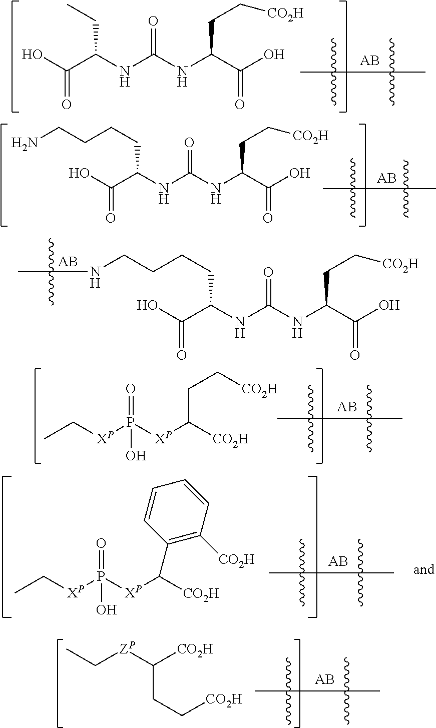

wherein
X^{P} and Y^{P} are each independently CH₂, O, NH, or S; and
Z^{P} is O, CH₂, NR⁶, S(O), S(O)₂, —S(O)₂O, —OS(O)₂, or OS(O)₂O.

### Envelope Glycoprotein GP120

In certain embodiments, the gp120 binding ligand is compound disclosed in WO 2001/062255 or WO 2003/072028. In certain embodiments, the gp120 binding ligand is a

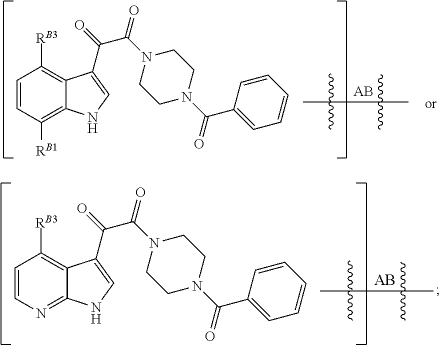

wherein
R^{B1} is selected from hydrogen, C₁—C alkyl, —O—C₁-C₆alkyl, aryl, and heteroaryl;
in certain embodiments, R^{B1} is phenyl, naphthyl, pyridyl (2-, 3- or 4-pyridyl group), thiazolyl (2-, 4- or 5-thiazole), isothiazolyl, oxazolyl (2-, 4- or 5-oxazole), isoxazolyl, furanyl (2- or 3-furan) or thiophenyl (2- or 3-thiophene);
R^{B2} is hydrogen or C₁-C₃alkyl; and
R^{B3} is selected from hydrogen, C₁-C₆alkyl, —O—, —NR⁶—, —S(O)—, —S(O)₂—, —S(O)₂O—, and —OS(O)₂O—.

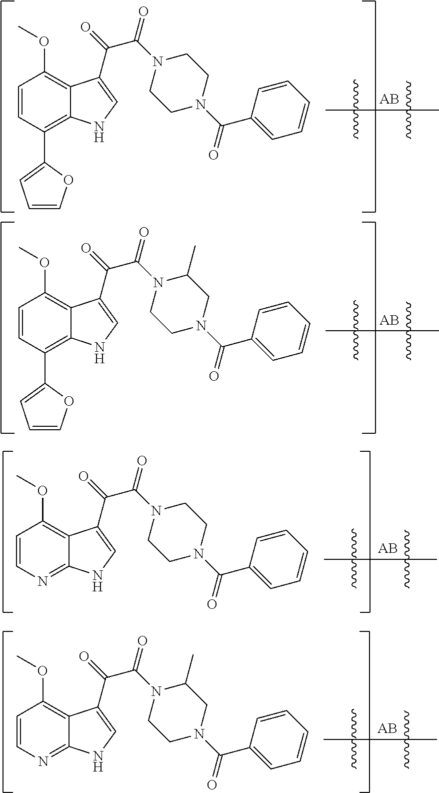

In certain embodiments, the Target Protein is selected from Serum amyloid P component, amyloid precursor protein, C reactive protein (CRP), an N-methyl-D-aspartate (NMD A) receptor, a-synuclein, LAPP, transthyretin, and combinations thereof. In some embodiments, the Target Protein is selected from a calcitonin gene-related peptide (CGRP), a CGRP receptor, an N-methyl-D-aspartate (NMD A) receptor, myeloperoxidase (MPO), IAPP, transthyretin, extracellular tau, beta-amyloid, amyloid precursor protein, prion protein, and a-synuclein. In some embodiments, the Extracellular Protein Targeting Ligand binds to extracellular tau, beta-amyloid, amyloid precursor protein, prion protein, a-synuclein, or a combination thereof.

In certain embodiments, the Extracellular Protein Targeting Ligand comprises one of the following amino acid sequences that binds extracellular tau:

VY-WIW:
(SEQ ID NO: 401)
SVWIWYE, (Seidler, P. M. et al, Journal of Biological Chemistry,
2019, 29: 16451-16464);
or
IN-M4:
(SEQ ID NO: 402)
DVWIINKKLK, (Seidler, P. M. et al , Journal of Biological Chemistry,
2019, 29: 16451-16464), wherein SEQ ID NOs 401 and 402 can be attached to
the Linker through the C or N terminus.

In certain embodiments, the Extracellular Protein Targeting Ligand comprises one of the following amino acid sequences that targets amyloid beta:

NCAM1(N):
(SEQ ID NO: 403)
MLRTKDLIWTLFFLGTAVS-NH₂, (Henning- Knechtel, A. et
al, Cell Reports Physical Science, 2020, 26:100014);
N-Pr:
(SEQ ID NO: 404)
MLRTKDLIWTLFFLGTAVSKKRPKP-NH₂, (Henning- Knechtel, A.
et al, Cell Reports Physical Science, 2020, 26:100014);
or
N-Ab:
(SEQ ID NO: 405)
MLRTKDLIWTLFFLGTAVSKKLVFF-NFb, (Henning- Knechtel, A. et al,
Cell Reports Physical Science, 2020, 26:100014), wherein SEQ ID NOs: 403-405
can be attached to the Linker through the C or N terminus.

In certain embodiments, the amino end of any of SEQ ID NOs:403-405 binds to the Linker group or the LPR1 binding motif. In other embodiments, the carboxylic acid end of any of SEQ ID NOs:403-405 binds to the Linker. In certain embodiments, the carboxylic acid terminus of any of SEQ ID NOs:403-405 is a carboxamide group and the amine terminus is covalently linked to the Linker.

In certain embodiments, the Target Protein Binding Ligand binds to the N-methyl-D-aspartate (NMD A) receptor. Non limiting examples of a Target Protein Binding Ligand that bind to the N-methyl-D-aspartate (NMD A) receptor include

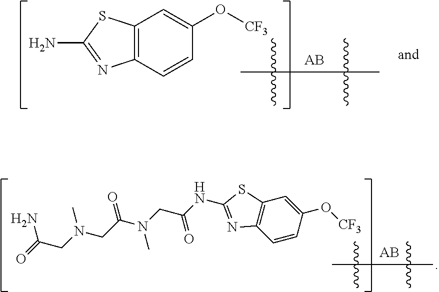

In certain embodiments, the Target Protein Binding Ligand binds prions. Non limiting examples of a Target Protein Binding Ligand that binds a prion include

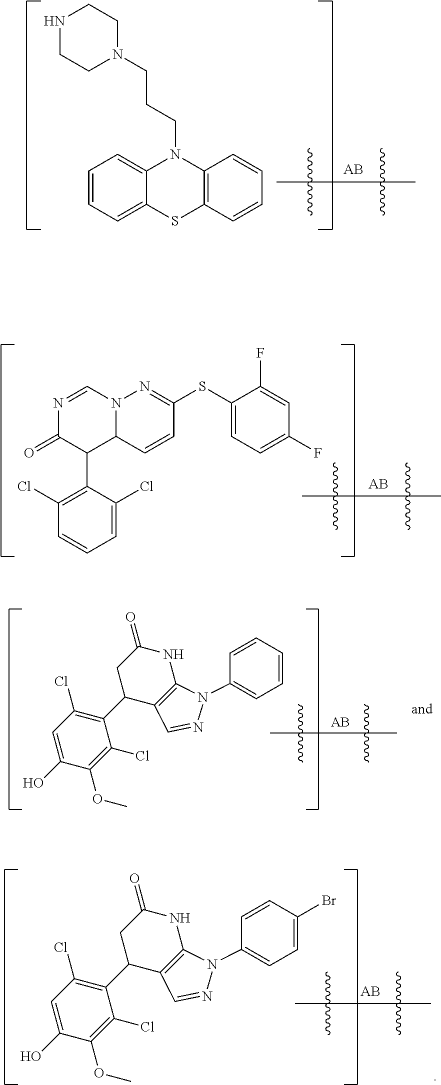

In certain embodiments, the Target Protein Binding Ligand binds prions. Non limiting examples of a Target Protein Binding Ligand that binds a prion include

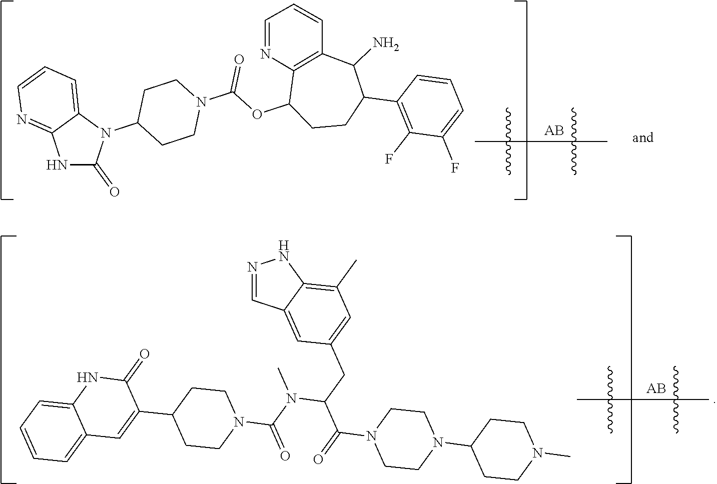

### CD16a (FcγRIII)

In certain embodiments, the Target Protein Binding Ligand binds CD16a. In some embodiments, CD16a is a high-affinity form, e.g., 158V. In some embodiments, CD16a is a low-5 affinity form, e.g., 158F. Nonlimiting examples of ligands that bind CD16a include

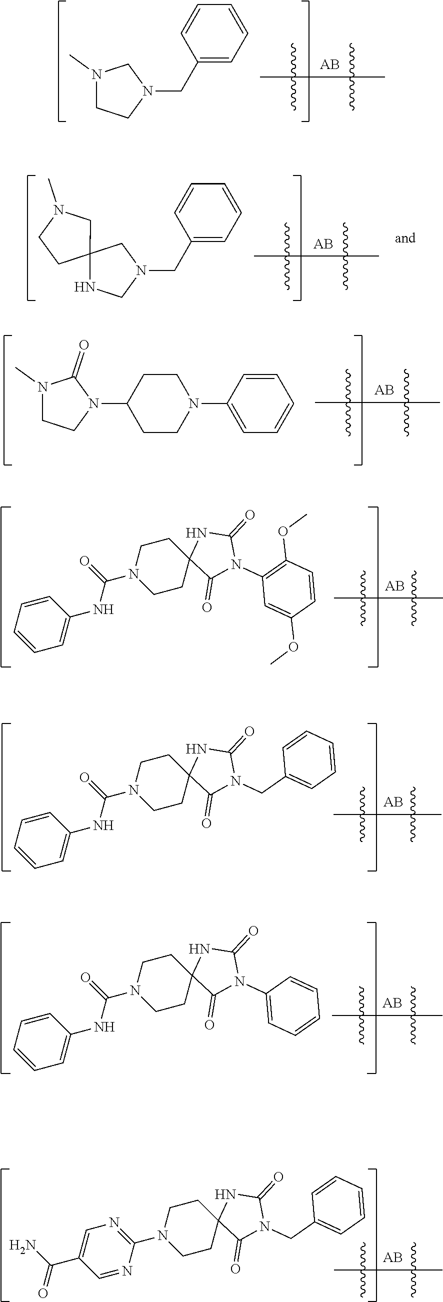

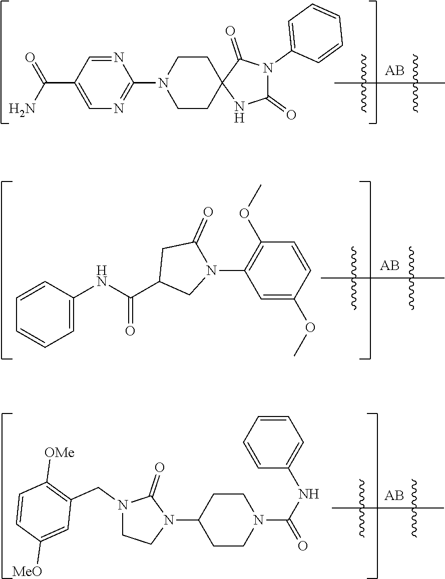

### Toll-Like Receptors

Toll-like receptors (TLRs) are receptors that recognize pathogens via pathogen- associated molecular patterns. These receptors are a major component of immune response, and activation of TLRs results in proinflammatory signaling. Decreasing TLR activity may be beneficial in the treatment of diseases such as rheumatoid arthritis and systemic lupus erythematosus.

In certain embodiments, the Extracellular Protein Targeting Ligand binds TLRs. In certain embodiments the TLR ligand is Rintatolimod, SMP-105, IPH-3102, CBL^{B502}, MGN-1706, IMO-2055, ANA773, OM-174, ISS1018, Agatolimod, 852A, Imiquimod or Cadi-05. In certain embodiments the TLR ligand is Imiquimod. In certain embodiments, the TLR ligand is a compound described in Bioorg. Med. Chem. Lett. (2010) 6384; Wu, et al. Proc Natl Acad Sci USA, 2007; 104 (10):3990-5; Gorden, et al., The Journal of Immunology, 2005; 174 (3):1259-68; and Reece, et al., or Proc Natl Acad Sci USA, 2005; 102 (42):15190-4.

In certain embodiments, the TLR ligand is selected from

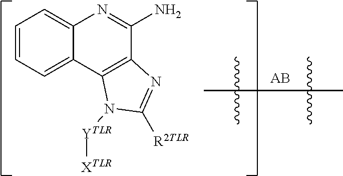

Wherein
R^{2TLR} is C₁-C₄alkyl, —C₁-C₄—NR⁶—C₁-C₄-, —C₁-C₄—O—C₁-C₄-, and —C₁-C₄—S—C₁-C₄-;
Y^{TLR} is selected from

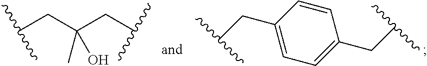

and
X^{TLR} is selected from OH, SH, NH₂, or NH(C₁-C₃alkyl).

In certain embodiments, the TLR ligand is selected from

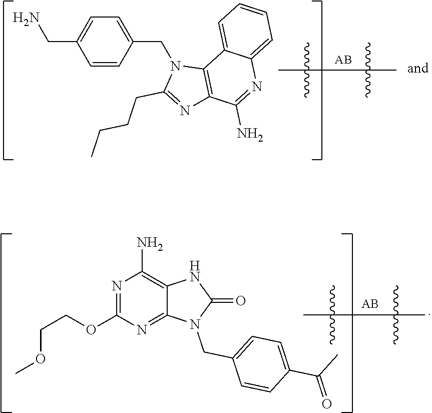

### Soluble FLT-1 (VEGFR-1)

Soluble fms-like tyrosine kinase 1 (sFLT-1), an antiangiogenic protein implicated in the pathogenesis of preeclampsia. This receptor binds VEGFA, VEGFB, and placental growth factor and controls angiogenesis in healthy and diseased tissues. sFLT-1 is upregulated in women with preeclampsia and high levels of sFLT-1 is a cause of maternal hypertension of proteinuria. During placentation, a critical process is remodeling of certain arteries to support the pregnancy. One such artery is the spiral artery (SpA). SpA remodeling involves apoptosis of certain mother cells followed by the production of specialized fetal trophoblast cells in combination with uterine natural killer (uNK) cells surrounding the SpAs. In preeclampsia pregnancies, SpA remodeling is poor and the reduced vasodilation during preeclampsia affects placental perfusion.

In certain embodiments, the Extracellular Protein Targeting Ligand binds sFLT-1. In certain embodiments, the sFLT-1 binding ligand is a fragment or homolog of VEGFA, VEGFB, or placental growth factor (Barleon, B. et al “Mapping the Sites for Ligand Binding and Receptor Dimerization at the Extracellular Domain of the Vascular Endothelial Growth Factor Receptor FLT-1” Protein Chemistry and Structure 1997, 272 (16), 10382-10388).

Soluble Endoglin (sEng)

sEng (soluble Eng), caused by Eng shedding from endothelial cell surface, has antiangiogenic effects in pregnant women, which can lead to preeclampsia. It has been hypothesized that this occurs via binding to circulating TGF-β1, a protein involved in homeostasis of angiogenic processes. sEng is used as a biomarker of preeclampsia during pregnancy, in particular during the second trimester. In certain embodiments, the Extracellular Protein Targeting Ligand binds sEng.

### TNF-alpha (TNF-α)

In some embodiments, the Target Extracellular Protein is human TNF-α (UniProtKB-P01375 (TNFA_HUMAN)). TNF-α is a pro-inflammatory cytokine active in the bodily immune response and serious inflammatory diseases. TNF-α has been implicated in a number of disorders, including but not limited to rheumatoid arthritis, inflammatory bowel disease, graft-vs-host disease, ankylosing spondylitis, psoriasis, hidradenitis suppurativa, refractory asthma, systemic lupus erthyematosus, diabetes, and the induction of cachexia.

The Protein Data Bank website provides the crystal structure of TNF-α searchable by 6RMJ (Valentinis, B., et al., Int. J. Mol. Sci., 2019, 20); 5UUI (Carrington et al., Biophys J., 2017, 113 371-380); 600Y, 600Z and 6OPO(O'Connell, J., et al., Nat. Commun., 2019,10 5795-5795); and 5TSW (Cha, S. S., J Biol Chem., 1998, 273 2153-2160); as well as the crystal structure of TNF-α bound to various compounds searchable by 5YOY (Ono et al., Protein Sci., 2018, 27 1038-1046); 2AZ5 (He., M. M., et al., Science, 2005, 310:1022-1025); 5WUX (Lee, J. U., Int J Mol Sci., 2017, 18); 5MU8 (Blevitt et al., J Med Chem., 2017, 60 3511-3517); 4Y60 (Feldman J. L., et al., Biochemistry, 2015, 54 3037-3050); 3WD5 (Hu, S., et al., J Biol Chem, 2013, 288 27059-27067); and 4G3Y (Liang, S. Y., J Biol Chem., 2013, 288 13799-13807).

Representative TNF-α Targeting Ligands are provided in FIG. 1. Additional TNF-α Targeting Ligands can be found in, for example, U.S. Pat. No. 8,541,572; J Chem Inf Model. 2017 May 22; 57 (5):1101-1111; each of which is incorporated by reference herein.

In certain embodiments the TNF-alpha Targeting Ligand is selected from:

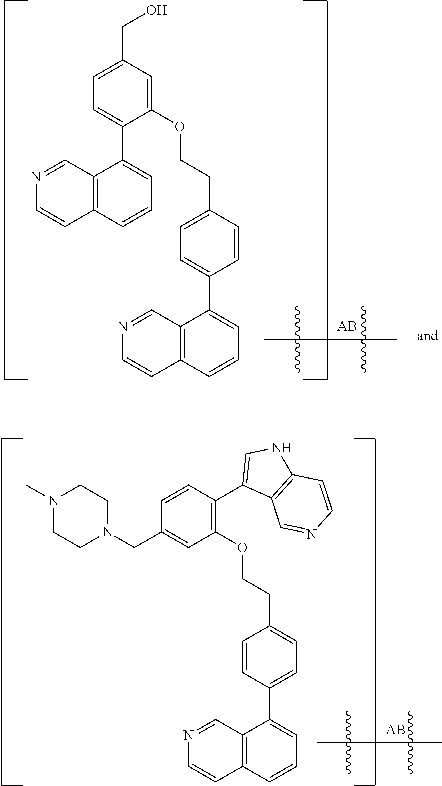

In certain embodiments the TNF-alpha Targeting Ligand is selected from:

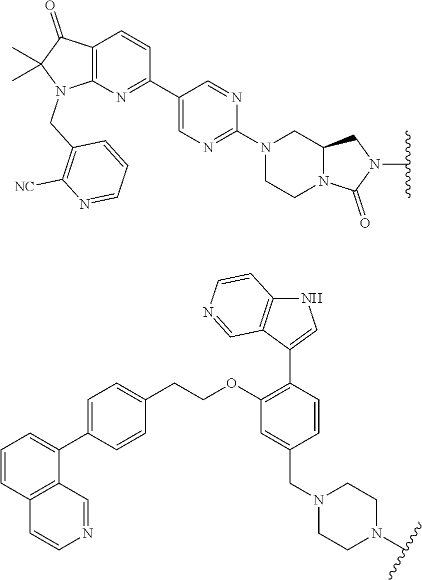 [SMILES: *N1C[C@@H]2CN(c3ncc(-c4ccc5c(n4)N(Cc4cccnc4C#N)C(C)(C)C5=O)cn3)CCN2C1=O ; *N1CCN(Cc2ccc(-c3c[nH]c4ccncc34)c(OCCc3ccc(-c4cccc5ccncc45)cc3)c2)CC1]

In alternative embodiments, the TNF-alpha Targeting Ligand comprises the polypeptide STPTRYS (SEQ ID NO: 406) (Guangdong Yixue 2008, 29 (1):55-57). In certain embodiments, the TNF-alpha Targeting Ligand comprises the polypeptide CALWHWWHC (SEQ ID NO: 407) or C (T/S) WLHWWAC (SEQ ID NO: 408) (Diyi Daxue Xuebao 2002, 22 (7):597-599). In certain embodiments, the TNF-alpha Targeting Ligand comprises any Tbab protein described in Zhu, et al., 2016, Protein Sci.25:2066-2075. In certain embodiments, the TNF-alpha Targeting Ligand comprises the polypeptide (L/M) HEL (Y/F) (L/M) X (W/Y/F), as described in Zhang, et al., 2003; Biochem. Biophys. Res. Commun.310:1181-1187. In certain embodiments, the TNF-alpha Targeting Ligand comprises one of the polypeptides:

SEQ ID NO: 409
D-DDDEK QLKER WYKRW LEYLD EFKKN (DHPT-9)
SEQ ID NO: 410
D-TEEEK QLKEW WYKHW QEYLE EFKKN (DHPT-91) (Yang,
et al., 2019, FEBS Lett.593: 1292-1302).

In certain embodiments, the TNF-alpha Targeting Ligand comprises TNFR1 or TNFR2 (Yang & Yang, 2013, Fenxi Huaxue/Chinese J. Anal. Chem.41:664-669). In certain embodiments, the TNF-alpha Targeting Ligand comprises anticachexin C1 and/or C2 (Lian, et al., 2013, J. Am. Chem. Soc. 135:11990-11995).

In certain embodiments, the TNF-alpha Targeting Ligand comprises adalimumab, infliximab, etanercept, golimumab, and/or certolizumab. In certain embodiments, the TNF-alpha Targeting Ligand comprises the 29.2 kDa scFv identified in Safarpour, et al., 2018, Iran. J. Pharm. Res. 17:743-752. In certain embodiments, the TNF-alpha Targeting Ligand comprises GACPPCLWQVLCGGSGSGSG (SEQ ID NO: 411) (which can be, in a non-limiting example, tris-bromomethyl mesitylene core sulfur linked; Luzi, et al., 2015, Protein Eng. Des. Sel.28:45-52). In certain embodiments, the TNF-alpha Targeting Ligand comprises any affibodies (˜60 amino acids) identified in Löfdahl, et al., 2009, N. Biotechnol.26:251-259. In certain embodiments, the TNF-alpha Targeting Ligand comprises any affibodies identified in Kronqvist, et al., 2008, Protein Eng. Des. Sel.21:247-255. In certain embodiments, the TNF-alpha Targeting Ligand comprises any affibodies identified in Jonsson, et al., 2009, Biotechnol. Appl. Biochem.54:93-103. In certain embodiments, the TNF-alpha Targeting Ligand comprises the bispecific albumin/TNF binding polypeptide identified in Nilvebrant, et al., 2011, PLoS One 6. In certain embodiments, the TNF-alpha Targeting Ligand comprises the ubiquitin-based artificial binding protein identified in Hoffmann, et al., 2012, PLoS One 7:2-11. In certain embodiments, the TNF-alpha Targeting Ligand comprises HIHDDLLRYYGW linear (SEQ ID NO: 412) or tetra branched peptide (SEQ ID NOS:502-504) identified in Brunetti, et al., 2014, Molecules 19:7255-7268.

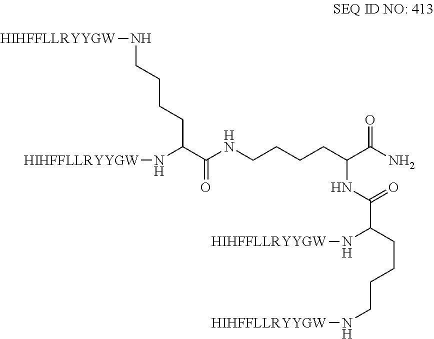

In certain embodiments, the TNF-alpha Targeting Ligand comprises any TNF-binding peptides (P51 and P52) identified in Alizadeh, et al., 2017, Eur. J. Pharm. Sci.96:490-498.

In certain embodiments, the TNF-alpha Targeting Ligand comprises the scFv antibody identified in Alizadeh, et al., 2015, Adv. Pharm. Bull.5:661-666. In certain embodiments, the TNF-alpha Targeting Ligand comprises any TNF binding peptide recited in WO 2006/053568 (such as but not limited to KRWSRYF (SEQ ID NO: 414), which may in certain embodiments be polyvalent), which is incorporated herein in its entirety by reference. In certain embodiments, the TNF-alpha Targeting Ligand comprises any TNF binding peptide recited in WO 2015/055597 (such as but not limited to HIHDDLLRYYGW (SEQ ID NO: 415), which may in certain embodiments be polyvalent), which is incorporated herein in its entirety by reference. In certain embodiments, the TNF-alpha Targeting Ligand comprises YCWSQYLCY (SEQ ID NO: 416) as identified in Arthritis & Rheumatism 2007, 56 (4):1164-74. In certain embodiments, the TNF-alpha Targeting Ligand comprises DFLPHYKNTSLGHRP (SEQ ID NO: 417) as identified in Chirinos-Rojas, et al., 1998, J. Immunol. 161:5621-5626. In certain embodiments, the TNF-alpha Targeting Ligand comprises YCLYQSWCY (SEQ ID NO: 418). In certain embodiments, the TNF-alpha Targeting Ligand is its reduced form (i.e., with an internal disulfide bond). In certain embodiments, the TNF-alpha Targeting Ligand is its oxidized form (i.e., without an internal disulfide bond).

In certain embodiments, the TNF binder comprises a compound of formula

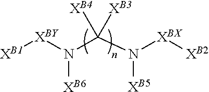

wherein:
X^{B1} and X^{B2} are independently selected from the group consisting of

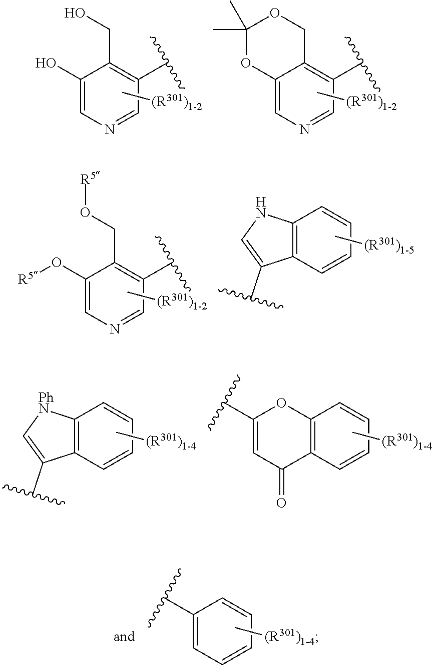

R⁵″ is independently hydrogen or optionally substituted C₁-C₄ alkyl;
X^{B3} and X^{B4} are independently hydrogen or optionally substituted C₁-C₄ alkyl
X^{BX} and X^{BY} are independently carbonyl or CH₂;
n is 2, 3, or 4;
X^{B5} and X^{B6} are independently hydrogen or optionally substituted C₁-C₄ alkyl, or
R³ and R⁴ can combine to form a heterocyclyl ring.

In certain embodiments, the TNF-alpha Targeting Ligand comprises any compound disclosed in U.S. Pat. No. 10,266,532, which is incorporated herein in its entirety by reference. In certain embodiments, the TNF-alpha Targeting Ligand comprises any compound disclosed in U.S. Pat. No. 9,879,016, which is incorporated herein in its entirety by reference. In certain embodiments, the TNF-alpha Targeting Ligand comprises any compound disclosed in WO 2008/142623, which is incorporated herein in its entirety by reference. In certain embodiments, the TNF-alpha Targeting Ligand is

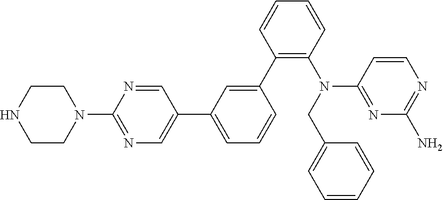 [SMILES: Nc1nccc(N(Cc2ccccc2)c2ccccc2-c2cccc(-c3cnc(N4CCNCC4)nc3)c2)n1]

In certain embodiments, the TNF-alpha Targeting Ligand is of the formula:

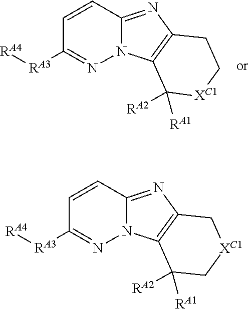

wherein
X^{C1} is CH₂ or O;
R^{A1} is

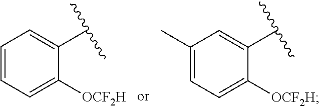

R^{A2} is selected from the group consisting of

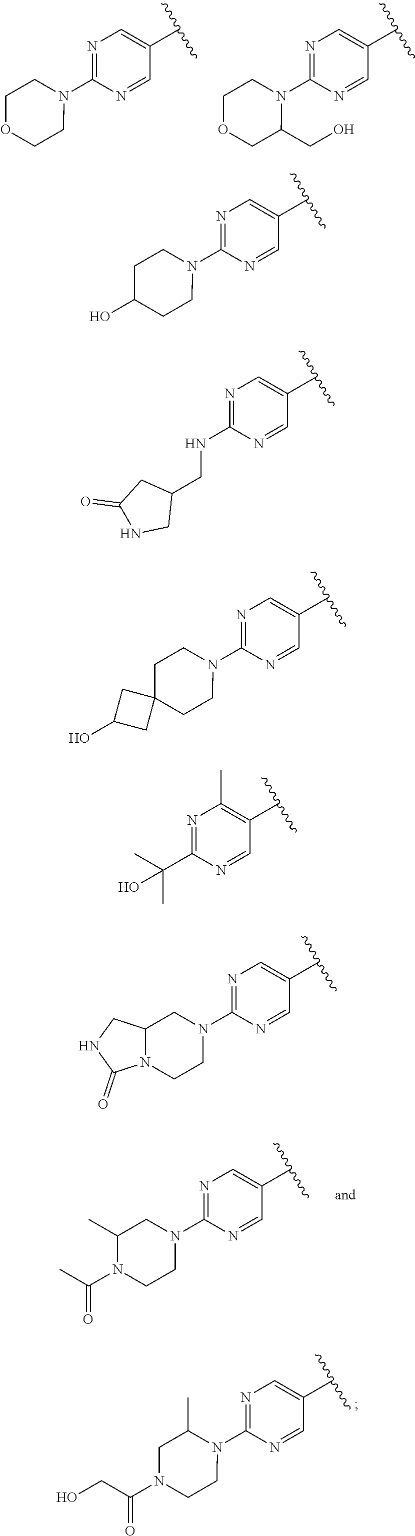

R^{A3} is selected from the group consisting of

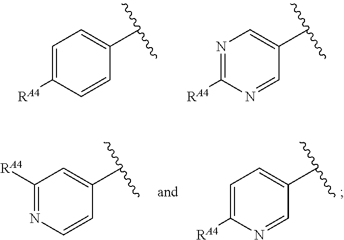

and
R^{A4} is selected from the group consisting of

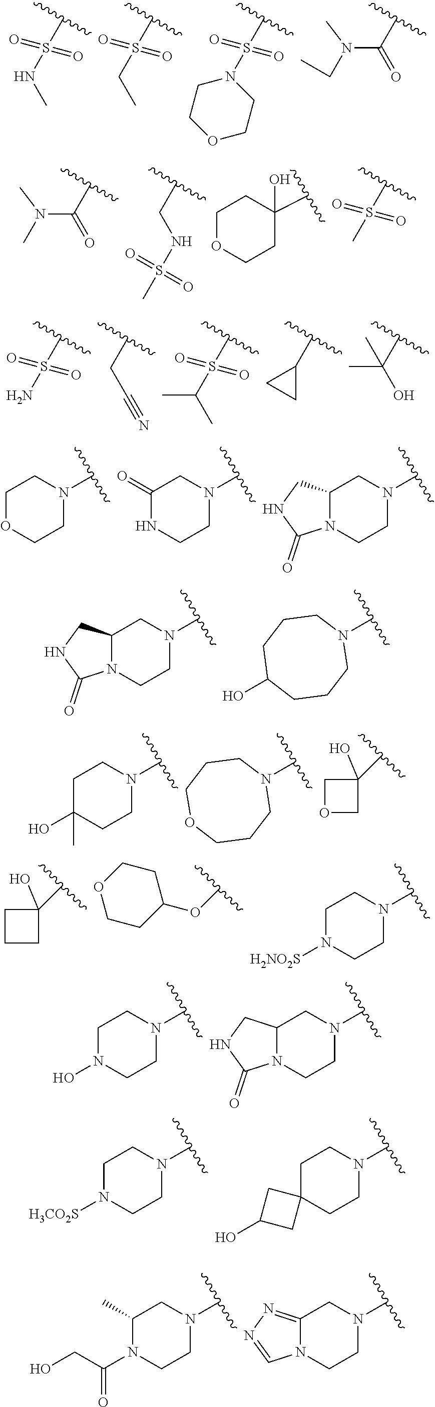

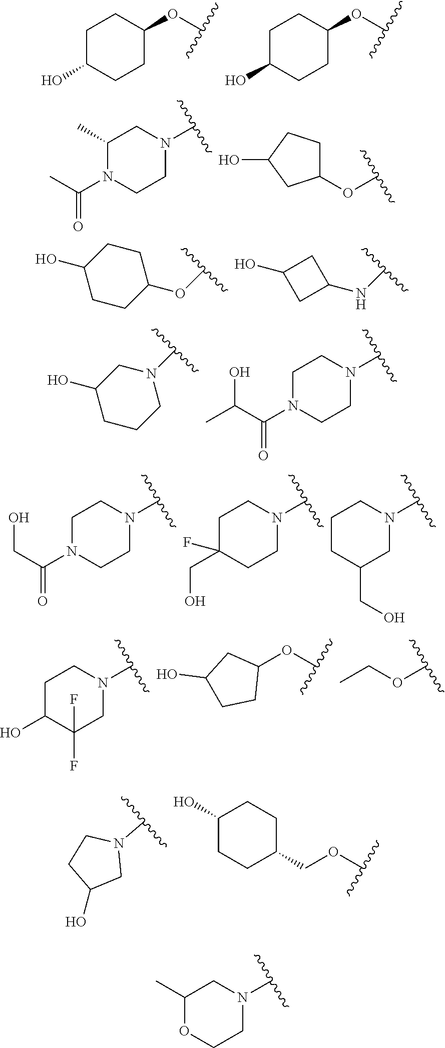

In certain embodiments, the TNF-alpha Targeting Ligand is selected from the group consisting of 2-(5-(1-(2-methoxyphenyl)-2,3-dihydro-1H-benzo[d]pyrrolo[1,2-a]imidazol-7-yl)pyrimidin-2-yl)propan-2-ol; 4-(3-fluorophenyl)-7-(2-morpholinopyrimidin-5-yl)-3,4-dihydro-1H-benzo[4,5]imidazo[2,1-c][1,4]oxazine; (R)-1-phenyl-7-(2-((tetrahydro-2H-pyran-4-yl)oxy)pyridin-4-yl)-2,3-dihydro-1H-benzo[d]pyrrolo[1,2-a]imidazole; (S)-2-(2-morpholinopyrimidin-5-yl)-9-phenyl-8,9-dihydro-6H-pyrido[3′,2′:4,5]imidazo[2,1-c][1,4]oxazine; 2-(5-(8-methyl-8-phenyl-7,8-dihydro-6H-pyrrolo[2′,1′: 2,3]imidazo[4,5-b]pyridin-2-yl)pyrimidin-2-yl)propan-2-ol; 2-(5-(1-(tetrahydro-2H-pyran-4-yl)-2,3-dihydro-1H-benzo[d]pyrrolo[1,2-a]imidazol-7-yl)pyrimidin-2-yl)propan-2-ol; (S)-7-(2-((1R,6S)-3,10-diazabicyclo[4.3.1]decan-10-yl)pyrimidin-5-yl)-4-phenyl-3,4-dihydro-1H-benzo[4,5]imidazo[2,1-c][1,4]oxazine; (S)-7-(5-(4-phenyl-3,4-dihydro-1H-benzo[4,5]imidazo[2,1-c][1,4]oxazin-7-yl)pyrimidin-2-yl)-7-azaspiro[3.5]nonan-2-amine; 7-(5-((S)-4-phenyl-3,4-dihydro-1H-benzo[4,5]imidazo[2,1-c][1,4]oxazin-7-yl)pyrimidin-2-yl)hexahydroimidazo[1,5-a]pyrazin-3 (2H)-one; 7-(5-(8-(2-methoxyphenyl)-7,8-dihydro-6H-cyclopenta[4,5]imidazo[1,2-b]pyridazin-2-yl)pyrimidin-2-yl)hexahydroimidazo[1,5-a]pyrazine-3 (2H)-one; 1-(5-(8-phenyl-7,8-dihydro-6H-cyclopenta[4,5]imidazo[1,2-b]pyridazin-2-yl)pyrimidin-2-yl)piperidin-4-ol; 2-(5-(4-(2-methoxyphenyl)-3,4-dihydro-1H-pyran [3′,4′:4,5]imidazo[1,2-a]pyridin-7-yl)pyrimidin-2-yl)propan-2-ol; (S)-7-(5-((R)-8-phenyl-7,8-dihydro-6H-pyrrolo[2′,1′:2,3]imidazo[4,5-b]pyridin-2-yl)pyrimidin-2-yl)hexahydroimidazo[1,5-a]pyrazin-3 (2H)-one; (R)-7-(5-((R)-1-phenyl-2,3-dihydro-1H-benzo[d]pyrrolo[1,2-a]imidazol-7-yl)pyrimidin-2-yl)hexahydroimidazo[1,5-a]pyrazin-3 (2H)-one; 2-(5-(8-(pyridin-2-yl)-7,8-dihydro-6H-pyrrolo[2′,1′: 2,3]imidazo[4,5-b]pyridin-2-yl)pyrimidin-2-yl)propan-2-ol; 2-(5-(1-(pyridin-2-yl)-2,3-dihydro-1H-benzo[d]pyrrolo[1,2-a]imidazol-7-yl)pyrimidin-2-yl)propan-2-ol; (S)-7-(5-((S)-4-phenyl-3,4-dihydro-1H-benzo[4,5]imidazo[2,1-c][1,4]oxazin-7-yl)pyrimidin-2-yl)hexahydroimidazo[1,5-a]pyrazin-3 (2H)-one; (R)-7-(5-((S)-4-phenyl-3,4-dihydro-1H-benzo[4,5]imidazo[2,1-c][1,4]oxazin-7-yl)pyrimidin-2-yl)hexahydroimidazo[1,5-a]pyrazin-3 (2H)-one; 7-(5-(9-(2-methoxyphenyl)-6,7,8,9-tetrahydrobenzo[4,5]imidazo[1,2-a]pyridin-2-yl)pyrimidin-2-yl)-7-azaspiro[3.5]nonan-4-(5-(9-(2-methoxyphenyl)-6,7,8,9-tetrahydrobenzo[4,5]imidazo[1,2-a]pyridin-2-2-ol; yl)pyrimidin-2-yl)-1,4-oxazepane; 7-(5-(9-(2-methoxyphenyl)-6,7,8,9-tetrahydrobenzo[4,5]imidazo[1,2-a]pyridin-2-yl)pyrimidin-2-yl)hexahydroimidazo[1,5-a]pyrazin-3 (2H)-one; 1-(5-(9-(2-methoxyphenyl)-6,7,8,9-tetrahydrobenzo[4,5]imidazo[1,2-a]pyridin-2-yl)pyrimidin-2-yl)-4-methylpiperidin-4-ol; (4-fluoro-1-(5-(9-(2-methoxyphenyl)-6,7,8,9-tetrahydrobenzo[4,5]imidazo[1,2-a]pyridin-2-yl)pyrimidin-2-yl)piperidin-4-yl) methanol; (4-fluoro-1-(5-(9-(2-methoxyphenyl)-6,7,8,9-tetrahydrobenzo[4,5]imidazo[1,2-a]pyridin-2-yl)pyrimidin-2-yl)piperidin-4-yl) methanol; 1-(5-(9-(2-methoxyphenyl)-6,7,8,9-tetrahydrobenzo[4,5]imidazo[1,2-a]pyridin-2-yl)pyrimidin-2-yl)azepan-4-ol; 1-(5-(9-(2-methoxyphenyl)-6,7,8,9-tetrahydrobenzo[4,5]imidazo[1,2-a]pyridin-2-yl)pyrimidin-2-yl)piperidin-4-ol; 1-(5-(8-methyl-8-phenyl-7,8-dihydro-6H-pyrrolo[2′,1′:2,3]imidazo[4,5-b]pyridin-2-yl)pyrimidin-2-yl)piperidin-4-ol; 1-(5-(9-(3-fluorophenyl)-8,9-dihydro-6H-pyrido[3′,2′:4,5]imidazo[2,1-c][1,4]oxazin-2-yl)pyrimidin-2-yl)piperidin-4-ol; 7-(5-(8-methyl-8-phenyl-7,8-dihydro-6H-pyrrolo[2′,1′:2,3]imidazo[4,5-b]pyridin-2-yl)pyrimidin-2-yl)hexahydroimidazo[1,5-a]pyrazin-3 (2H)-one; 7-(5-(9-(3-fluorophenyl)-8,9-dihydro-6H-pyrido[3′,2′:4,5]imidazo[2,1-c][1,4]oxazin-2-yl)pyrimidin-2-yl)hexahydroimidazo[1,5-a]pyrazin-3 (2H)-one; 7-(5-(8-cyclohexyl-7,8-dihydro-6H-pyrrolo[2′,1′:2,3]imidazo[4,5-b]pyridin-2-yl)pyrimidin-2-yl)hexahydroimidazo[1,5-a]pyrazin-3 (2H)-one; 7-(5-((R)-8-cyclohexyl-7,8-dihydro-6H-pyrrolo[2′,1′: 2,3]imidazo[4,5-b]pyridin-2-yl)pyrimidin-2-yl)hexahydroimidazo[1,5-a]pyrazin-3 (2H)-one; 7-(5-((S)-4-(2-chlorophenyl)-3,4-dihydro-1H-benzo[4,5]imidazo[2,1-c][1,4]oxazin-7-yl)pyrimidin-2-yl)hexahydroimidazo[1,5-a]pyrazin-7-(5-(4-(3-chlorophenyl)-3,4-dihydro-1H-benzo[4,5]imidazo[2,1-c][1,4]oxazin-7-3 (2H)-one; yl)pyrimidin-2-yl)hexahydroimidazo[1,5-a]pyrazin-3 (2H)-one; 7-(5-(4-(2-fluorophenyl)-3,4-dihydro-1H-benzo[4,5]imidazo[2,1-c][1,4]oxazin-7-yl)pyrimidin-2-yl)hexahydroimidazo[1,5-a]pyrazin-3 (2H)-one; 4-(5-(1-(2-methoxyphenyl)-2,3-dihydro-1H-cyclopenta[4,5]imidazo[1,2-a]pyridin-7-yl)pyrimidin-2-yl)morpholine; (R)-7-(5-((R)-9-phenyl-8,9-dihydro-6H-pyrano[3′,4′:4,5]imidazo[1,2-b]pyridazin-2-yl)pyrimidin-2-yl)hexahydroimidazo[1,5-a]pyrazin-3 (2H)-one; (R)-7-(5-((R)-4-phenyl-3,4-dihydro-1H-pyrano[3′,4′:4,5]imidazo[1,2-a]pyridin-7-yl)pyrimidin-2-yl)hexahydroimidazo[1,5-a]pyrazin-3 (2H)-one; 1-(5-(9-(2-methoxyphenyl)-6,7,8,9-tetrahydroimidazo[1,2-a:5,4-b′]dipyridin-2-yl)pyrimidin-2-yl)piperidin-4-ol; 7-(5-(8-(2-methoxyphenyl)-7,8-dihydro-6H-pyrrolo[2′,1′:2,3]imidazo[4,5-b]pyridin-2-yl)pyrimidin-2-yl)hexahydroimidazo[1,5-a]pyrazin-3 (2H)-one; 1-(5-(8-(2-methoxyphenyl)-7,8-dihydro-6H-pyrrolo[2′,1′:2,3]imidazo[4,5-b]pyridin-2-yl)pyrimidin-2-yl)piperidin-4-ol; (S)-1-(5-(9-(2-methoxyphenyl)-8,9-dihydro-6H-pyrido[3′,2′:4,5]imidazo[2,1-c][1,4]oxazin-2-yl)pyrimidin-2-yl)piperidin-4-ol; 7-(5-((S)-9-phenyl-8,9-dihydro-6H-pyrido[3′,2′:4,5]imidazo[2,1-c][1,4]oxazin-2-yl)pyrimidin-2-yl)hexahydroimidazo[1,5-a]pyrazin-1 (5H)-one; (S)-1-(5-(9-phenyl-8,9-dihydro-6H-pyrido[3′,2′:4,5]imidazo[2,1-c][1,4]oxazin-2-yl)pyrimidin-2-yl)piperidin-4-ol; (S)-4-(5-(9-phenyl-8,9-dihydro-6H-pyrido[3′,2′:4,5]imidazo[2,1-c][1,4]oxazin-2-yl)pyrimidin-2-(S)-2-(5-(9-phenyl-8,9-dihydro-6H-pyrido[3′,2′:4,5]imidazo[2,1-yl)piperazin-2-one; c][1,4]oxazin-2-yl)pyrimidin-2-yl)propan-2-ol; (S)-7-(5-((R)-1-phenyl-2,3-dihydro-1H-benzo[d]pyrrolo[1,2-a]imidazol-7-yl)pyrimidin-2-yl)hexahydroimidazo[1,5-a]pyrazin-3 (2H)-one; (R)-3-(5-(1-phenyl-2,3-dihydro-1H-benzo[d]pyrrolo[1,2-a]imidazol-7-yl)pyrimidin-2-yl)oxetan-3-ol; (R)-1-(5-(1-phenyl-2,3-dihydro-1H-benzo[d]pyrrolo[1,2-a]imidazol-7-yl)pyrimidin-2-yl)cyclobutanol; (R)-4-(5-(1-phenyl-2,3-dihydro-1H-benzo[d]pyrrolo[1,2-a]imidazol-7-yl)pyrimidin-2-yl)tetrahydro-2H-pyran-4-ol; (R)-7-(5-((R)-1-phenyl-2,3-dihydro-1H-benzo[d]pyrrolo[1,2-a]imidazol-7-yl)pyrimidin-2-yl)hexahydroimidazo[1,5-a]pyrazin-3 (2H)-one; 2-(5-(4-(2,6-dichlorophenyl)-3,4-dihydro-1H-benzo[4,5]imidazo[2,1-c][1,4]oxazin-7-yl)pyrimidin-2-yl)propan-2-ol; (S)-2-(5-(4-(2-methoxyphenyl)-3,4-dihydro-1H-benzo[4,5]imidazo[2,1-c][1,4]oxazin-7-yl)pyrimidin-2-yl)propan-2-ol; 7-(5-((S)-4-phenyl-3,4-dihydro-1H-benzo[4,5]imidazo[2,1-c][1,4]oxazin-7-yl)pyrimidin-2-yl)hexahydroimidazo[1,5-a]pyrazin-3 (2H)-one; 7-(5-(4-(2-chlorophenyl)-3,4-dihydro-1H-benzo[4,5]imidazo[2,1-c][1,4]oxazin-7-yl)pyrimidin-2-yl)hexahydroimidazo[1,5-a]pyrazin-3 (2H)-one; 7-(5-(1,2′,3,3′-tetrahydrospiro[benzo[4,5]imidazo[2,1-c][1,4]oxazine-4,1′- inden]-7-yl)pyrimidin-2-yl)hexahydroimidazo[1,5-a]pyrazin-3 (2H)-one; (S)-2-hydroxy-1-(4-(5-(4-phenyl-3,4-dihydro-1H-benzo[4,5]imidazo[2,1-c][1,4]oxazin-7-yl)pyrimidin-2-yl)piperazin-1-yl)ethanone; 7-(5-((S)-4-phenyl-3,4-dihydro-1H-benzo[4,5]imidazo[2,1-c][1,4]oxazin-7-yl)pyrimidin-2-yl)-7-azaspiro[3.5]nonan-1-ol; (R)-1-(5-(8-(3-fluorophenyl)-7,8-dihydro-6H-pyrrolo[2′,1′:2,3]imidazo[4,5-b]pyridin-2-yl)pyrimidin-2-yl)piperidin-4-ol; (R)-1-(5-(8-phenyl-7,8-dihydro-6H-pyrrolo[2′,1′: 2,3]imidazo[4,5-b]pyridin-2-yl)pyrimidin-2-yl)piperidin-4-ol; (R)-4-(5-(8-phenyl-7,8-dihydro-6H-pyrrolo[2′,1′:2,3]imidazo[4,5-b]pyridin-2-yl)pyrimidin-2-yl)morpholine; (S)-2-(5-(1-(2,5-dimethylphenyl)-2,3-dihydro-1H-benzo[d]pyrrolo[1,2-a]imidazol-7-yl)pyrimidin-2-yl)propan-2-ol; (R)-2-(5-(1-(2,5-dimethylphenyl)-2,3-dihydro-1H-benzo[d]pyrrolo[1,2-a]imidazol-7-yl)pyrimidin-2-yl)propan-2-ol; (S)-4-(3-fluorophenyl)-7-(2-morpholinopyrimidin-5-yl)-3,4-dihydro-1H-benzo[4,5]imidazo[2,1-c][1,4]oxazine; 7-(5-(8-(2,5-dimethylphenyl)-7,8-dihydro-6H-pyrrolo[2′,1′: 2,3]imidazo[4,5-b]pyridin-2-yl)pyrimidin-2-yl)hexahydroimidazo[1,5-a]pyrazin-3 (2H)-one compound with ethane (1:1); 7-(5-(8-(3-fluorophenyl)-7,8-dihydro-6H-pyrrolo[2′,1′:2,3]imidazo[4,5-b]pyridin-2-yl)pyrimidin-2-yl)hexahydroimidazo[1,5-a]pyrazin-3 (2H)-one; (R)-1-(5-(1-phenyl-2,3-dihydro-1H-benzo[d]pyrrolo[1,2-a]imidazol-7-yl)pyrimidin-2-yl)piperidin-4-ol; (R)-4-(5-(1-phenyl-2,3-dihydro-1H-benzo[d]pyrrolo[1,2-a]imidazol-7-yl)pyrimidin-2-yl)thiomorpholine 1,1-dioxide;-(5-(8-phenyl-7,8-dihydro-6H-pyrrolo[2′,1′:2,3]imidazo[4,5-b]pyridin-2-yl)pyrimidin-2-yl)hexahydroimidazo[1,5-a]pyrazin-3 (2H)-one; 7-(5-((R)-1-phenyl-2,3-dihydro-1H-benzo[d]pyrrolo[1,2-a]imidazol-7-yl)pyrimidin-2-yl)hexahydroimidazo[1,5-a]pyrazin-3 (2H)-one; 3,3-difluoro-1-(5-((R)-1-phenyl-2,3-dihydro-1H-benzo[d]pyrrolo[1,2-a]imidazol-7-yl)pyrimidin-2-yl)piperidin-4-ol; (S)-2-(5-(4-(2-(difluoromethoxy)phenyl)-3,4-dihydro-1H-benzo[4,5]imidazo[2,1-c][1,4]oxazin-7-yl)pyrimidin-2-yl)propan-2-ol; (S)-2-(5-(9-(2-methoxyphenyl)-6,7,8,9-tetrahydrobenzo[4,5]imidazo[1,2-a]pyridin-2-yl)pyrimidin-2-yl)propan-2-ol; (R)-7-(5-(1-phenyl-2,3-dihydro-1H-benzo[d]pyrrolo[1,2-a]imidazol-7-yl)pyrimidin-2-yl)-7-azaspiro[3.5]nonan-2-ol; 1-(5-(8-(2,5-dimethylphenyl)-7,8-dihydro-6H-pyrrolo[2′,1′:2,3]imidazo[4,5-b]pyridin-2-yl)pyrimidin-2-yl)piperidin-4-ol; 4-(5-(10-(2-methoxyphenyl)-7,8,9,10-tetrahydro-6H-cyclohepta[4,5]imidazo[1,2-a]pyridin-2-yl)pyrimidin-2-yl)morpholine; (R)-4-(5-(1-phenyl-2,3-dihydro-1H-benzo[d]pyrrolo[1,2-a]imidazol-7-yl)pyrimidin-2-yl)morpholine; 7-(5-((S)-4-phenyl-3,4-dihydro-1H-benzo[4,5]imidazo[2,1-c][1,4]oxazin-7-yl)pyrimidin-2-yl)hexahydroimidazo[1,5-a]pyrazin-3 (2H)-one; 4-(5-(8-(2,5-dimethylphenyl)-7,8-dihydro-6H-pyrrolo[2′,1′:2,3]imidazo[4,5-b]pyridin-2-yl)pyrimidin-2-yl)piperazin-2-one; 2-(5-(1-(2,5-dimethylphenyl)-2,3-dihydro-1H-benzo[d]pyrrolo[1,2-a]imidazol-7-yl)pyrimidin-2-yl)propan-2-ol; 3,3-difluoro-1-(5-((S)-4-phenyl-3,4-dihydro-1H-benzo[4,5]imidazo[2,1-c][1,4]oxazin-7-yl)pyrimidin-2-yl)piperidin-4-ol; 7-(5-(4-(3-fluorophenyl)-3,4-dihydro-1H-benzo[4,5]imidazo[2,1-c][1,4]oxazin-7-yl)pyrimidin-2-yl)hexahydroimidazo[1,5-a]pyrazin-3 (2H)-one; (R)-2-(5-(1-(2-methoxyphenyl)-2,3-dihydro-1H-cyclopenta[4,5]imidazo[1,2-a]pyridin-7-yl)pyrimidin-2-yl)propan-2-ol; 7-(4-(isopropylsulfonyl)phenyl)-1-phenyl-2,3-dihydro-1H-benzo[d]pyrrolo[1,2-a]imidazole; 2-(5-(9-(2-methoxyphenyl)-6,7,8,9-tetrahydrobenzo[4,5]imidazo[1,2-a]pyridin-2-yl)pyrimidin-2-yl)propan-2-ol; 4-(5-(8-(2,5-dimethylphenyl)-7,8-dihydro-6H-pyrrolo[2′,1′:2,3]imidazo[4,5-b]pyridin-2-yl)pyrimidin-2-yl)morpholine; (S)-7-(5-(4-phenyl-3,4-dihydro-1H-benzo[4,5]imidazo[2,1-c][1,4]oxazin-7-yl)pyrimidin-2-yl)-7-azaspiro[3.5]nonan-2-ol; 4-(5-(9-(2-methoxyphenyl)-6,7,8,9-tetrahydrobenzo[4,5]imidazo[1,2-a]pyridin-2-yl)pyrimidin-2-yl)morpholine; (S)-1-(5-(4-phenyl-3,4-dihydro-1H-benzo[4,5]imidazo[2,1-c][1,4]oxazin-7-yl)pyrimidin-2-yl)piperidin-4-ol; 1-(5-(9-phenyl-6,7,8,9-tetrahydroimidazo[1,2-a:5,4-b′]dipyridin-2-yl)pyrimidin-2-yl)piperidin-4-ol; (R)-2-(5-(1-phenyl-2,3-dihydro-1H-benzo[d]pyrrolo[1,2-a]imidazol-7-yl)pyrimidin-2-yl)propan-2-ol; 4-(5-(8-(3-fluorophenyl)-7,8-dihydro-6H-pyrrolo[2′,1′:2,3]imidazo[4,5-b]pyridin-2-yl)pyrimidin-2-yl)morpholine; 2-(5-(10-(2-methoxyphenyl)-7,8,9,10-tetrahydro-6H-cyclohepta[4,5]imidazo[1,2-a]pyridin-2-yl)pyrimidin-2-yl)propan-2-ol; N-methyl-4-(1-phenyl-2,3-dihydro-1H-benzo[d]pyrrolo[1,2-a]imidazol-7-yl)benzenesulfonamide; 1-(5-(8-(3-fluorophenyl)-7,8-dihydro-6H-pyrrolo[2′,1′:2,3]imidazo[4,5-b]pyridin-2-yl)pyrimidin-2-yl)piperidin-4-ol; 7-(4-(S)-7-(2-(ethylsulfonyl)phenyl)-1-phenyl-2,3-dihydro-1H-benzo[d]pyrrolo[1,2-a]imidazole; morpholinopyrimidin-5-yl)-4-phenyl-3,4-dihydro-1H-benzo[4,5]imidazo[2,1-c][1,4]oxazine; 4-(5-(8-phenyl-7,8-dihydro-6H-pyrrolo[2′,1′:2,3]imidazo[4,5-b]pyridin-2-yl)pyrimidin-2-yl)morpholine; 4-(5-(8-phenyl-7,8-dihydro-6H-pyrrolo[2′,1′:2,3]imidazo[4,5-b]pyridin-2-yl)pyrimidin-2-yl)morpholine; (S)-7-(5-(4-phenyl-3,4-dihydro-1H-benzo[4,5]imidazo[2,1-c][1,4]oxazin-7-yl)pyrimidin-2-yl)-7-azaspiro[3.5]nonan-2-ol; 1-(5-(8-phenyl-7,8-dihydro-6H-pyrrolo[2′,1′: 2,3]imidazo[4,5-b]pyridin-2-yl)pyrimidin-2-yl)piperidin-4-ol; (S)-7-(2-(1,4-oxazepan-4-yl)pyrimidin-5-yl)-4-phenyl-3,4-dihydro-1H-benzo[4,5]imidazo[2,1-c][1,4]oxazine; 4-(5-(1-phenyl-2,3-dihydro-1H-benzo[d]pyrrolo[1,2-a]imidazol-7-yl)pyrimidin-2-yl)morpholine; 3,3-difluoro-1-(5-(4-(3-fluorophenyl)-3,4-dihydro-1H-benzo[4,5]imidazo[2,1-c][1,4]oxazin-7-yl)pyrimidin-2-yl)piperidin-4-ol; (1-(5-((S)-4-phenyl-3,4-dihydro-1H-benzo[4,5]imidazo[2,1-c][1,4]oxazin-7-yl)pyrimidin-2-yl)piperidin-3-yl) methanol; 1-(5-((S)-4-phenyl-3,4-dihydro-1H-benzo[4,5]imidazo[2,1-c][1,4]oxazin-7-yl)pyrimidin-2-yl)azepan-4-ol; (S)-4-(5-(4-phenyl-3,4-dihydro-1H-benzo[4,5]imidazo[2,1-c][1,4]oxazin-7-yl)pyrimidin-2-yl)piperazine-1-sulfonamide; N-(4-(1-phenyl-2,3-dihydro-1H-benzo[d]pyrrolo[1,2-a]imidazol-7-yl)benzyl) methanesulfonamide; 2-(5-(1-(2-methoxyphenyl)-2,3-dihydro-1H-cyclopenta[4,5]imidazo[1,2-a]pyridin-7-yl)pyrimidin-2-yl)propan-2-ol; 7-(4-(methylsulfonyl)phenyl)-1-phenyl-2,3-dihydro-1H-benzo[d]pyrrolo[1,2-a]imidazole; 7-(2-(1,4-oxazepan-4-yl)pyrimidin-5-yl)-4-(3-fluorophenyl)-3,4-dihydro-1H-benzo[4,5]imidazo[2,1-c][1,4]oxazine; 4-(1-phenyl-2,3-dihydro-1H-benzo[d]pyrrolo[1,2-a]imidazol-7-yl)benzenesulfonamide; (4-(5-((S)-4-phenyl-3,4-dihydro-1H-benzo[4,5]imidazo[2,1-c][1,4]oxazin-7-yl)pyrimidin-2-yl)morpholin-2-yl) methanol; (S)-4-(5-(4-phenyl-3,4-dihydro-1H-benzo[4,5]imidazo[2,1-c][1,4]oxazin-7-yl)pyrimidin-2-yl)thiomorpholine 1,1-dioxide; 4-(5-(9-phenyl-6,7,8,9-tetrahydroimidazo[1,2-a:5,4-b′]dipyridin-2-yl)pyrimidin-2-yl)morpholine; (R)-4-(5-(1-phenyl-1,2,3,4-tetrahydrobenzo[4,5]imidazo[1,2-a]pyridin-8-yl)pyrimidin-2-yl)morpholine; 2-(5-(1-(3-fluorophenyl)-2,3-dihydro-1H-benzo[d]pyrrolo[1,2-a]imidazol-7-yl)pyrimidin-2-yl)propan-2-ol; (S)-(4-fluoro-1-(5-(4-phenyl-3,4-dihydro-1H-benzo[4,5]imidazo[2,1-c][1,4]oxazin-7-yl)pyrimidin-2-yl)piperidin-4-yl) methanol; 4-(5-(9-(3-chlorophenyl)-6,7,8,9-tetrahydrobenzo[4,5]imidazo[1,2-a]pyridin-2-yl)pyrimidin-2-yl)morpholine; (R)-2-(5-(1-phenyl-1,2,3,4-tetrahydrobenzo[4,5]imidazo[1,2-a]pyridin-8-yl)pyrimidin-2-yl)propan-2-ol; (S)-7-(2-(1′-methyl-[4,4′-bipiperidin]-1-yl)pyrimidin-5-yl)-4-phenyl-3,4-dihydro-1H-benzo[4,5]imidazo[2,1-c][1,4]oxazine; 2-(5-(9-phenyl-6,7,8,9-tetrahydroimidazo[1,2-a:5,4-b′]dipyridin-2-yl)pyrimidin-2-yl)propan-2-ol; 7-(2-Morpholinopyrimidin-5-yl)-4-phenyl-3,4-dihydro-1H-benzo[4,5]imidazo[2,1-c][1,4]oxazine; (4S)-7-(2-(2-methylmorpholino)pyrimidin-5-yl)-4-phenyl-3,4-dihydro-1H-benzo[4,5]imidazo[2,1-c][1,4]oxazine; 7-(5-(4-(3-fluorophenyl)-3,4-dihydro-1H-benzo[4,5]imidazo[2,1-c][1,4]oxazin-7-yl)pyrimidin-2-yl)-7-azaspiro[3.5]nonan-2-ol; 4-(5-(1-phenyl-1,2,3,4-tetrahydrobenzo[4,5]imidazo[1,2-a]pyridin-8-yl)pyrimidin-2-yl)morpholine; Ethyl 2-[[5-[9-(2-methoxyphenyl)-6,7,8,9-tetrahydropyrido[1,2-a]benzimidazol-2-yl]pyrimidin-(S)-7-(2-(5,6-dihydro-[1,2,4]triazolo[4,3-a]pyrazin-7 (8H)-yl)pyrimidin-5-2-yl]amino]acetate; yl)-4-phenyl-3,4-dihydro-1H-benzo[4,5]imidazo[2,1-c][1,4]oxazine; 2-(5-(8-(3-fluorophenyl)-7,8-dihydro-6H-pyrrolo[2′,1′:2,3]imidazo[4,5-b]pyridin-2-yl)pyrimidin-2-yl)propan-2-ol; (S)-7-(2-(4-(methylsulfonyl)piperazin-1-yl)pyrimidin-5-yl)-4-phenyl-3,4-dihydro-1H-benzo[4,5]imidazo[2,1-c][1,4]oxazine; 2-(5-(8-phenyl-7,8-dihydro-6H-pyrrolo[2′,1′:2,3]imidazo[4,5-b]pyridin-2-yl)pyrimidin-2-yl)propan-2-ol; 2-(4-(1-phenyl-2,3-dihydro-1H-benzo[d]pyrrolo[1,2-a]imidazol-7-yl)phenyl)acetonitrile; 4-(5-(8-phenyl-7,8-dihydro-6H-pyrrolo[2′,1′:2,3]imidazo[4,5-b]pyridin-2-yl)pyrimidin-2-yl)piperazin-2-one; 7-(2-cyclopropylpyrimidin-5-yl)-1-phenyl-2,3-dihydro-1H-benzo[d]pyrrolo[1,2-a]imidazole; (S)-4-(5-(4-phenyl-3,4-dihydro-1H-benzo[4,5]imidazo[2,1-c][1,4]oxazin-7-yl)pyrimidin-2-yl)piperazin-2-one; 2-((5-(9-(2-methoxyphenyl)-6,7,8,9-tetrahydrobenzo[4,5]imidazo[1,2-a]pyridin-2-yl)pyridin-2-yl)oxy) acetic acid; 7-(6-(ethylsulfonyl)pyridin-3-yl)-1-phenyl-2,3-dihydro-1H-benzo[d]pyrrolo[1,2-a]imidazole; 4-(5-(8-(3-fluorophenyl)-7,8-dihydro-6H-pyrrolo[2′,1′:2,3]imidazo[4,5-b]pyridin-2-yl)pyrimidin-2-yl)piperazin-2-one; 10-(3-fluorophenyl)-2-(2-morpholinopyrimidin-5-yl)-7,8,9,10-tetrahydro-6H-cyclohepta[4,5]imidazo[1,2-a]pyridin-10-ol; 4-(5-(9-phenyl-6,7,8,9-tetrahydroimidazo[1,2-a:5,4-b′]dipyridin-2-yl)pyrimidin-2-yl)piperazin-2-one; (S)-6-(5-(4-phenyl-3,4-dihydro-1H-benzo[4,5]imidazo[2,1-c][1,4]oxazin-7-yl)pyrimidin-2-yl)-6-azaspiro[3.4]octan-2-ol; N,N-dimethyl-4-(1-phenyl-2,3-dihydro-1H-benzo[d]pyrrolo[1,2-a]imidazol-7-yl)benzamide; N-ethyl-N-methyl-4-(1-phenyl-2,3-dihydro-1H-benzo[d]pyrrolo[1,2-a]imidazol-7-yl)benzamide; 7-(6-morpholinopyridin-3-yl)-4-phenyl-3,4-dihydro-1H-benzo[4,5]imidazo[2,1-c][1,4]oxazine; 2-(5-(1-phenyl-1,2,3,4-tetrahydrobenzo[4,5]imidazo[1,2-a]pyridin-8-yl)pyrimidin-2-yl)propan-2-ol; 2-(5-(1-Phenyl-2,3-dihydro-1H-benzo[d]pyrrolo[1,2-a]imidazol-7-yl)pyrimidin-2-yl)propan-2-ol; (S)-4-(2-hydroxyethyl)-1-(5-(4-phenyl-3,4-dihydro-1H-benzo[4,5]imidazo[2,1-c][1,4]oxazin-7-yl)pyrimidin-2-yl)piperidin-4-ol; (S)-7-(2-(2-oxa-7-azaspiro[3.5]nonan-7-yl)pyrimidin-5-yl)-4-phenyl-3,4-dihydro-1H-benzo[4,5]imidazo[2,1-c][1,4]oxazine; 2-(1-(5-((S)-4-phenyl-3,4-dihydro-1H-benzo[4,5]imidazo[2,1-c][1,4]oxazin-7-yl)pyrimidin-2-yl)piperidin-3-yl)acetic acid; 7-(5-methyl-6-morpholinopyridin-3-yl)-4-phenyl-3,4-dihydro-1H-benzo[4,5]imidazo[2,1-c][1,4]oxazine; 7-(5-(2-methyl-1H-imidazol-1-yl)pyrazin-2-yl)-4-phenyl-3,4-dihydro-1H-benzo[4,5]imidazo[2,1-c][1,4]oxazine; 2-(5-(1-cyclohexyl-2,3-dihydro-1H-benzo[d]pyrrolo[1,2-a]imidazol-7-yl)pyrimidin-2-yl)propan-2-ol; 2-(5-(4-phenyl-3,4-dihydro-1H-benzo[4,5]imidazo[2,1-c][1,4]oxazin-7-yl)pyrimidin-2-yl)propan-2-ol; (S)-(4-(methylsulfonyl)-1-(5-(4-phenyl-3,4-dihydro-1H-benzo[4,5]imidazo[2,1-c][1,4]oxazin-7-yl)pyrimidin-2-yl)piperidin-4-yl) methanol; 1-(1-(5-((S)-4-phenyl-3,4-dihydro-1H-benzo[4,5]imidazo[2,1-c][1,4]oxazin-7-yl)pyrimidin-2-yl)piperidin-4-yl)ethanol; (S)-4-(5-(4-phenyl-3,4-dihydro-1H-benzo[4,5]imidazo[2,1-c][1,4]oxazin-7-yl)pyrimidin-2-yl)-1,4-diazepan-2-one; 2-(4-(1-phenyl-2,3-dihydro-1H-benzo[d]pyrrolo[1,2-a]imidazol-7-yl) phenoxy) acetonitrile; (S)—N-(1-(5-(4-phenyl-3,4-dihydro-1H-benzo[4,5]imidazo[2,1-c][1,4]oxazin-7-yl)pyrimidin-2-yl)piperidin-4-yl) methanesulfonamide; (S)-3-(1-(5-(4-phenyl-3,4-dihydro-1H-benzo[4,5]imidazo[2,1-c][1,4]oxazin-7-yl)pyrimidin-2-yl)piperidin-4-yl)propanoic acid; 4-phenyl-7-(6-(trifluoromethyl)pyridin-3-yl)-3,4-dihydro-1H-benzo[4,5]imidazo[2,1-c][1,4]oxazine; 4-(5-(1-phenyl-1,2,3,4-tetrahydrobenzo[4,5]imidazo[1,2-a]pyridin-8-yl)pyrimidin-2-yl)piperazin-2-one; 7-(5-fluoro-6-methoxypyridin-3-yl)-4-phenyl-3,4-dihydro-1H-benzo[4,5]imidazo[2,1-c][1,4]oxazine; N,N-dimethyl-5-(1-phenyl-2,3-dihydro-1H-benzo[d]pyrrolo[1,2-a]imidazol-7-yl)pyridin-2-amine; 7-(2-methylpyridin-4-yl)-1-phenyl-2,3-dihydro-1H-benzo[d]pyrrolo[1,2-a]imidazole; (4S)-4-phenyl-7-(2-(2-(trifluoromethyl)morpholino)pyrimidin-5-yl)-3,4-dihydro-1H-benzo[4,5]imidazo[2,1-c][1,4]oxazine; 7-(5-((S)-4-phenyl-3,4-dihydro-1H-benzo[4,5]imidazo[2,1-c][1,4]oxazin-7-yl)pyrimidin-2-yl)-2,7-diazaspiro[4.4]nonan-1-one; N-cyclopentyl-5-(4-phenyl-3,4-dihydro-1H-benzo[4,5]imidazo[2,1-c][1,4]oxazin-7-yl)pyrimidin-2-amine; 7-(2-(1H-pyrazol-1-yl)pyrimidin-5-yl)-4-phenyl-3,4-dihydro-1H-benzo[4,5]imidazo[2,1-c][1,4]oxazine; (4S)-7-(2-(2,6-dimethylmorpholino)pyrimidin-5-yl)-4-phenyl-3,4-dihydro-1H-benzo[4,5]imidazo[2,1-c][1,4]oxazine; 7-(6-methylpyridin-3-yl)-1-phenyl-2,3-dihydro-1H-benzo[d]pyrrolo[1,2-a]imidazole; 7-(5-ethoxypyridin-3-yl)-1-phenyl-2,3-dihydro-1H-benzo[d]pyrrolo[1,2-a]imidazole; 2-(2-morpholinopyrimidin-5-yl)-9-(m-tolyl)-6,7,8,9-tetrahydrobenzo[4,5]imidazo[1,2-a]pyridin-9-ol; 7-(6-(methylthio)pyridin-3-yl)-1-phenyl-2,3-dihydro-1H-benzo[d]pyrrolo[1,2-a]imidazole; ethyl 2-((5-(10-(2-methoxyphenyl)-7,8,9,10-tetrahydro-6H-cyclohepta[4,5]imidazo[1,2-a]pyridin-2-yl)pyrimidin-2-yl)amino)acetate; (S)-3-(4-(5-(4-phenyl-3,4-dihydro-1H-benzo[4,5]imidazo[2,1-c][1,4]oxazin-7-yl)pyrimidin-2-yl)piperazin-1-yl)propan-1-ol; 9-(3-fluorophenyl)-2-(2-morpholinopyrimidin-5-yl)-6,7,8,9-tetrahydrobenzo[4,5]imidazo[1,2-a]pyridin-9-ol; 2-(2-morpholinopyrimidin-5-yl)-9-phenyl-6,7,8,9-tetrahydrobenzo[4,5]imidazo[1,2-a]pyridin-9-ol; 4-(2,5-difluorophenyl)-7-(2-morpholinopyrimidin-5-yl)-3,4-dihydro-1H-benzo[4,5]imidazo[2,1-c][1,4]oxazine; 1-phenyl-7-(6-(piperazin-1-yl)pyridin-3-yl)-2,3-dihydro-1H-benzo[d]pyrrolo[1,2-a]imidazole; 9-(2-Methoxyphenyl)-2-(2-morpholinopyrimidin-5-yl)-6,7,8,9-tetrahydrobenzo[4,5]imidazo[1,2-a]pyridin-9-ol; (S)-7-(2-(2-oxa-6-azaspiro[3.4]octan-6-yl)pyrimidin-5-yl)-4-phenyl-3,4-dihydro-1H-benzo[4,5]imidazo[2,1-c][1,4]oxazine; 7-(5-(1H-imidazol-1-yl)pyrazin-2-yl)-4-phenyl-3,4-dihydro-1H-benzo[4,5]imidazo[2,1-c][1,4]oxazine; 7-(furo[3,2-b]pyridin-6-yl)-4-phenyl-3,4-dihydro-1H-benzo[4,5]imidazo[2,1-c][1,4]oxazine; 10-(3-chlorophenyl)-2-(2-morpholinopyrimidin-5-yl)-7,8,9,10-tetrahydro-6H-cyclohepta[4,5]imidazo[1,2-a]pyridin-10-ol; N-ethyl-4-(1-phenyl-2,3-dihydro-1H-benzo[d]pyrrolo[1,2-a]imidazol-7-yl)benzamide; 2-(3-(1-phenyl-2,3-dihydro-1H-benzo[d]pyrrolo[1,2-a]imidazol-7-yl) phenoxy) acetonitrile; 2-(2-morpholinopyrimidin-5-yl)-10-(m-tolyl)-7,8,9,10-tetrahydro-6H-cyclohepta[4,5]imidazo[1,2-a]pyridin-10-ol; 2-((5-(10-(2-methoxyphenyl)-7,8,9,10-tetrahydro-6H-cyclohepta[4,5]imidazo[1,2-a]pyridin-2-yl)pyrimidin-2-yl)amino) acetic acid; N-cyclopropyl-4-(1-phenyl-2,3-dihydro-1H-benzo[d]pyrrolo[1,2-a]imidazol-7-yl)benzamide; 4-(1-phenyl-2,3-dihydro-1H-benzo[d]pyrrolo[1,2-a]imidazol-7-yl)benzamide; 1-(4-(5-(4-phenyl-3,4-dihydro-1H-benzo[4,5]imidazo[2,1-c][1,4]oxazin-7-yl)pyridin-2-yl)piperazin-1-yl)ethanone; 7-(6-(4-methylpiperazin-1-yl)pyridin-3-yl)-1-phenyl-2,3-dihydro-1H-benzo[d]pyrrolo[1,2-a]imidazole; 7-(benzo[d][1,3]dioxol-5-yl)-1-phenyl-2,3-dihydro-1H-benzo[d]pyrrolo[1,2-a]imidazole; 9-(3-fluoro-2-methylphenyl)-2-(2-morpholinopyrimidin-5-yl)-6,7,8,9-tetrahydrobenzo[4,5]imidazo[1,2-a]pyridin-9-ol; 8-Phenyl-2-(4-(pyrimidin-2-yl)piperazin-1-yl)-7,8-dihydro-6H-pyrrolo[2′,1′:2,3]imidazo[4,5-b]pyridine; 9-(4-fluorophenyl)-2-(2-morpholinopyrimidin-5-yl)-6,7,8,9-tetrahydrobenzo[4,5]imidazo[1,2-a]pyridin-9-ol; 7-(5-((S)-4-phenyl-3,4-dihydro-1H-benzo[4,5]imidazo[2,1-c][1,4]oxazin-7-yl)pyrimidin-2-yl)tetrahydro-1H-oxazolo[3,4-a]pyrazin-3 (5H)-one; 2-((5-(9-(2-methoxyphenyl)-6,7,8,9-tetrahydrobenzo[4,5]imidazo[1,2-a]pyridin-2-yl)pyrimidin-2-yl)amino) acetic acid; 10-(4-fluorophenyl)-2-(2-morpholinopyrimidin-5-yl)-7,8,9,10-tetrahydro-6H-cyclohepta[4,5]imidazo[1,2-a]pyridin-10-ol; 9-(3-chlorophenyl)-2-(2-morpholinopyrimidin-5-yl)-6,7,8,9-tetrahydrobenzo[4,5]imidazo[1,2-a]pyridin-9-ol; N-cyclopropyl-5-(4-phenyl-3,4-dihydro-1H-benzo[4,5]imidazo[2,1-c][1,4]oxazin-7-yl)pyrimidin-2-amine; 9-(3-chloro-5-fluorophenyl)-2-(2-morpholinopyrimidin-5-yl)-6,7,8,9-tetrahydrobenzo[4,5]imidazo[1,2-a]pyridin-9-ol; N,N-dimethyl-5-(4-phenyl-3,4-dihydro-1H-benzo[4,5]imidazo[2,1-c][1,4]oxazin-7-yl)pyrimidin-2-amine; 1-(5-(1-phenyl-1,2,3,4-tetrahydrobenzo[4,5]imidazo[1,2-a]pyridin-8-yl)pyrimidin-2-yl)piperidine-4-carboxylic acid; 7-(6-isopropoxypyridin-3-yl)-4-phenyl-3,4-dihydro-1H-benzo[4,5]imidazo[2,1-c][1,4]oxazine; 7-(6-isopropoxypyridin-3-yl)-4-phenyl-3,4-dihydro-1H-benzo[4,5]imidazo[2,1-c][1,4]oxazine; 4-(5-(6-phenyl-7,8-dihydro-6H-pyrrolo[11,2′: 1,2]imidazo[4,5-c]pyridin-3-yl)pyrimidin-2-yl)morpholine; 1-phenyl-7-(1-(pyridin-3-ylmethyl)-1H-pyrazol-4-yl)-2,3-dihydro-1H-benzo[d]pyrrolo[1,2-a]imidazole; 5-(4-phenyl-3,4-dihydro-1H-benzo[4,5]imidazo[2,1-c][1,4]oxazin-7-yl)picolinonitrile; 7-(4-methyl-3,4-dihydro-2H-pyrido[3,2-b][1,4]oxazin-7-yl)-4-phenyl-3,4-dihydro-1H-benzo[4,5]imidazo[2,1-c][1,4]oxazine; 2-(2-morpholinopyrimidin-5-yl)-9-(p-tolyl)-6,7,8,9-tetrahydrobenzo[4,5]imidazo[1,2-a]pyridin-9-ol; 7-(6-(methylsulfonyl)pyridin-3-yl)-4-phenyl-3,4-dihydro-1H-benzo[4,5]imidazo[2,1-c][1,4]oxazine; N-(2-methoxyethyl)-4-(1-phenyl-2,3-dihydro-1H-benzo[d]pyrrolo[1,2-a]imidazol-7-yl)benzamide; N-methyl-4-(1-phenyl-2,3-dihydro-1H-benzo[d]pyrrolo[1,2-a]imidazol-7-yl)benzamide; 4-phenyl-7-(6-(2,2,2-trifluoroethoxy)pyridin-3-yl)-3,4-dihydro-1H-benzo[4,5]imidazo[2,1-c][1,4]oxazine; 1-(5-(4-phenyl-3,4-dihydro-1H-benzo[4,5]imidazo[2,1-c][1,4]oxazin-7-yl)pyrimidin-2-yl)piperidine-4-carboxylic acid; 7-(5-methyl-6-(4-methylpiperazin-1-yl)pyridin-3-yl)-4-phenyl-3,4-dihydro-1H-benzo[4,5]imidazo[2,1-c][1,4]oxazine; 5-(9-(2-Methoxyphenyl)-6,7,8,9-yl)thiophene-2-carboxylic acid; 7-(6-(4-tetrahydrobenzo[4,5]imidazo[1,2-a]pyridin-2-methylpiperazin-1-yl)pyridin-3-yl)-4-phenyl-3,4-dihydro-1H-benzo[4,5]imidazo[2,1-c][1,4]oxazine; (S)-7-(2-(1′-methyl-[4,4′-bipiperidin]-1-yl)pyrimidin-5-yl)-4-phenyl-3,4-dihydro-1H-benzo[4,5]imidazo[2,1-c][1,4]oxazine; 7-(2-methoxypyrimidin-5-yl)-4-phenyl-3,4-dihydro-1H-benzo[4,5]imidazo[2,1-c][1,4]oxazine; 10-(3-chloro-5-fluorophenyl)-2-(2-morpholinopyrimidin-5-yl)-7,8,9,10-tetrahydro-6H-cyclohepta[4,5]imidazo[1,2-a]pyridin-10-ol; 3-(2-hydroxyethyl)-1-(5-((S)-4-phenyl-3,4-dihydro-1H-benzo[4,5]imidazo[2,1-c][1,4]oxazin-7-yl)pyrimidin-2-yl)pyrrolidin-3-ol; 4-(5-(9-(2-Methoxyphenyl)-6,7-dihydrobenzo[4,5]imidazo[1,2-a]pyridin-2-yl)pyrimidin-2-yl)morpholine; 10-(4-methoxyphenyl)-2-(2-morpholinopyrimidin-5-yl)-7,8,9,10-tetrahydro-6H-cyclohepta[4,5]imidazo[1,2-a]pyridin-10-ol; 7-(5-(1H-pyrazol-1-yl)pyrazin-2-yl)-4-phenyl-3,4-dihydro-1H-benzo[4,5]imidazo[2,1-c][1,4]oxazine; 1-phenyl-7-(pyridin-3-yl)-2,3-dihydro-1H-benzo[d]pyrrolo[1,2-a]imidazole; 5-(1-phenyl-2,3-dihydro-1H-benzo[d]pyrrolo[1,2-a]imidazol-7-yl)pyrimidin-2-amine; 2-(2-morpholinopyrimidin-5-yl)-10-(p-tolyl)-7,8,9,10-tetrahydro-6H-cyclohepta[4,5]imidazo[1,2-a]pyridin-10-ol; 5-(4-phenyl-3,4-dihydro-1H-benzo[4,5]imidazo[2,1-c][1,4]oxazin-7-yl)pyrimidine-2-carbonitrile; (R)-2-(5-(9-(2-methoxyphenyl)-6,7,8,9-tetrahydrobenzo[4,5]imidazo[1,2-a]pyridin-2-yl)pyrimidin-2-yl)propan-2-ol; (S)-7-(2-((R)-3-(methylsulfonyl)pyrrolidin-1-yl)pyrimidin-5-yl)-4-phenyl-3,4-dihydro-1H-benzo[4,5]imidazo[2,1-c][1,4]oxazine; 7-([1,2,5]oxadiazolo[3,4-b]pyridin-6-yl)-4-phenyl-3,4-dihydro-1H-benzo[4,5]imidazo[2,1-c][1,4]oxazine; 2-(5-(6-phenyl-7,8-dihydro-6H-pyrrolo[1,2′: 1,2]imidazo[4,5-c]pyridin-3-yl)pyrimidin-2-yl)propan-2-ol; 7-(2-methylpyrimidin-5-yl)-4-phenyl-3,4-dihydro-1H-benzo[4,5]imidazo[2,1-c][1,4]oxazine; 1-phenyl-7-(pyrimidin-5-yl)-2,3-dihydro-1H-benzo[d]pyrrolo[1,2-a]imidazole; 7-(6-methoxy-5-methylpyridin-3-yl)-4-phenyl-3,4-dihydro-1H-benzo[4,5]imidazo[2,1-c][1,4]oxazine; 10-(4-chlorophenyl)-2-(2-morpholinopyrimidin-5-yl)-7,8,9,10-tetrahydro-6H-cyclohepta[4,5]imidazo[1,2-a]pyridin-10-ol; 2-(3-(1-phenyl-2,3-dihydro-1H-benzo[d]pyrrolo[1,2-a]imidazol-7-yl)phenyl)acetonitrile; N-(3-(1-phenyl-2,3-dihydro-1H-benzo[d]pyrrolo[1,2-a]imidazol-7-yl)benzyl) methanesulfonamide; 7-(6-methylpyridin-3-yl)-4-phenyl-3,4-dihydro-1H-benzo[4,5]imidazo[2,1-c][1,4]oxazine; (S)-2-(5-(1-phenyl-1,2,3,4-tetrahydrobenzo[4,5]imidazo[1,2-a]pyridin-8-yl)pyrimidin-2-yl)propan-2-ol; (S)-7-(2-(4-(1-methylpiperidin-4-yl)piperazin-1-yl)pyrimidin-5-yl)-4-phenyl-3,4-dihydro-1H-benzo[4,5]imidazo[2,1-c][1,4]oxazine; 4-phenyl-7-(6-(piperazin-1-yl)pyridin-3-yl)-3,4-dihydro-1H-benzo[4,5]imidazo[2,1-c][1,4]oxazine; (4S)-7-(2-(3-(methylsulfonyl)pyrrolidin-1-yl)pyrimidin-5-yl)-4-phenyl-3,4-dihydro-1H-benzo[4,5]imidazo[2,1-c][1,4]oxazine; (S)-8-(5-(4-phenyl-3,4-dihydro-1H-benzo[4,5]imidazo[2,1-c][1,4]oxazin-7-yl)pyrimidin-2-yl)-1,3,8-triazaspiro[4.5]decan-4-one; 7-(6-isopropoxy-5-methylpyridin-3-yl)-4-phenyl-3,4-dihydro-1H-benzo[4,5]imidazo[2,1-c][1,4]oxazine; 7-(5-methylpyridin-3-yl)-1-phenyl-2,3-dihydro-H-benzo[d]pyrrolo[1,2-a]imidazole; N-methyl-3-(1-phenyl-2,3-dihydro-1H-benzo[d]pyrrolo[1,2-a]imidazol-7-yl)benzamide; 4-(1-phenyl-2,3-dihydro-1H-benzo[d]pyrrolo[1,2-a]imidazol-7-yl)-N-((tetrahydrofuran-2-yl)methyl)benzamide; (S)-4-(5-(1-phenyl-2,3-dihydro-1H-benzo[d]pyrrolo[1,2-a]imidazol-7-yl)pyrimidin-2-yl)morpholine; N-(5-(4-phenyl-3,4-dihydro-1H-benzo[4,5]imidazo[2,1-c][1,4]oxazin-7-yl)pyridin-2-yl)acetamide; N-ethyl-N-methyl-3-(1-phenyl-2,3-dihydro-1H-benzo[d]pyrrolo[1,2-a]imidazol-7-yl)benzamide; 5-(1-phenyl-2,3-dihydro-1H-benzo[d]pyrrolo[1,2-a]imidazol-7-yl)pyridin-2-amine; 7-(3-methyl-3H-imidazo[4,5-b]pyridin-6-yl)-4-phenyl-3,4-dihydro-1H-benzo[4,5]imidazo[2,1-c][1,4]oxazine; 10-(3,5-dimethoxyphenyl)-2-(2-morpholinopyrimidin-5-yl)-7,8,9,10-tetrahydro-6H-cyclohepta[4,5]imidazo[1,2-a]pyridin-10-ol; N,N-dimethyl-3-((5-(4-phenyl-3,4-dihydro-1H-benzo[4,5]imidazo[2,1-c][1,4]oxazin-7-yl)pyridin-2-yl)oxy) propan-1-amine; (3R,4R)-1-(5-((S)-4-phenyl-3,4-dihydro-1H-benzo[4,5]imidazo[2,1-c][1,4]oxazin-7-yl)pyrimidin-2-yl)pyrrolidine-3,4-diol; N-(2-(dimethylamino)ethyl)-3-(1-phenyl-2,3-dihydro-1H-benzo[d]pyrrolo[1,2-a]imidazol-7-yl)benzamide; 10-(3-methoxyphenyl)-2-(2-morpholinopyrimidin-5-yl)-7,8,9,10-tetrahydro-6H-cyclohepta[4,5]imidazo[1,2-a]pyridin-10-ol; 7-(1,5-dimethyl-1H-pyrazol-4-yl)-1-phenyl-2,3-dihydro-1H-benzo[d]pyrrolo[1,2-a]imidazole; N,N-dimethyl-3-(1-phenyl-2,3-dihydro-1H-benzo[d]pyrrolo[1,2-a]imidazol-7-yl)benzamide; 7-(3-(methylsulfonyl)phenyl)-1-phenyl-2,3-dihydro-1H-benzo[d]pyrrolo[1,2-a]imidazole; 4-phenyl-7-(pyrido[2,3-b]pyrazin-7-yl)-3,4-dihydro-1H-benzo[4,5]imidazo[2,1-c][1,4]oxazine; 1-methyl-5-(1-phenyl-2,3-dihydro-1H-benzo[d]pyrrolo[1,2-a]imidazol-7-yl)pyridin-2 (1H)-one; (3S,4S)-1-(5-((S)-4-phenyl-3,4-dihydro-1H-benzo[4,5]imidazo[2,1-c][1,4]oxazin-7-yl)pyrimidin-2-yl)pyrrolidine-3,4-diol; 4-phenyl-7-(pyrimidin-5-yl)-3,4-dihydro-1H-benzo[4,5]imidazo[2,1-c][1,4]oxazine; (S)-2-(5-(1-(2-Methoxyphenyl)-2,3-dihydro-1H-cyclopenta[4,5]imidazo[1,2-a]pyridin-7-yl)pyrimidin-2-yl)propan-2-ol; (S)-2-(5-(1-phenyl-2,3-dihydro-1H-benzo[d]pyrrolo[1,2-a]imidazol-7-yl)pyrimidin-2-yl)propan-2-ol; 2-(2-(2-(2-hydroxypropan-2-yl)pyrimidin-5-yl)-6,7,8,9-tetrahydrobenzo[4,5]imidazo[1,2-a]pyridin-9-yl) phenol; 4-(5-(6-phenyl-6,7,8,9-yl)piperazin-2-one; (S)-7-(2-(3-tetrahydroimidazo[1,2-a:4,5-b′]dipyridin-3-yl)pyrimidin-2-morpholinoazetidin-1-yl)pyrimidin-5-yl)-4-phenyl-3,4-dihydro-1H-benzo[4,5]imidazo[2,1-c][1,4]oxazine; 7-(5-(methylsulfonyl)pyridin-3-yl)-1-phenyl-2,3-dihydro-1H-benzo[d]pyrrolo[1,2-a]imidazole; (R)-7-(2-Morpholinopyrimidin-5-yl)-4-phenyl-3,4-dihydro-1H-benzo[4,5]imidazo[2,1-c][1,4]oxazine; 7-(2-methylpyridin-3-yl)-1-phenyl-2,3-dihydro-1H-benzo[d]pyrrolo[1,2-a]imidazole; 1-(5-(1-phenyl-1,2,3,4-tetrahydrobenzo[4,5]imidazo[1,2-a]pyridin-8-yl)pyrimidin-2-yl)azetidine-3-carboxylic acid; 7-(1-methyl-1H-pyrrol-3-yl)-1-phenyl-2,3-dihydro-1H-benzo[d]pyrrolo[1,2-a]imidazole; or N-(2-morpholinoethyl)-5-(4-phenyl-3,4-dihydro-1H-benzo[4,5]imidazo[2,1-c][1,4]oxazin-7-yl)pyridin-2-amine. In certain embodiments, the compound is selected from the group consisting of:7-(5-((R)-1-Phenyl-2,3-dihydro-1H-benzo[d]pyrrolo[1,2-a]imidazol-7-yl)pyrimidin-2-yl)hexahydroimidazo[1,5-a]pyrazin-3 (2H)-one; 3,3-difluoro-1-(5-((R)-1-phenyl-2,3-dihydro-1H-benzo[d]pyrrolo[1,2-a]imidazol-7-yl)pyrimidin-2-yl)piperidin-4-ol; (S)-2-(5-(4-(2-(Difluoromethoxy)phenyl)-3,4-dihydro-1H-benzo[4,5]imidazo[2,1-c][1,4]oxazin-7-yl)pyrimidin-2-yl)propan-2-ol; (S)-2-(5-(9-(2-methoxyphenyl)-6,7,8,9-tetrahydrobenzo[4,5]imidazo[1,2-a]pyridin-2-yl)pyrimidin-2-yl)propan-2-ol; (R)-7-(5-(1-phenyl-2,3-dihydro-1H-benzo[d]pyrrolo[1,2-a]imidazol-7-yl)pyrimidin-2-yl)-7-azaspiro[3.5]nonan-2-ol; 1-(5-(8-(2,5-dimethylphenyl)-7,8-dihydro-6H-pyrrolo[2′,1′:2,3]imidazo[4,5-b]pyridin-2-yl)pyrimidin-2-yl)piperidin-4-ol; 4-(5-(10-(2-methoxyphenyl)-7,8,9,10-tetrahydro-6H-cyclohepta[4,5]imidazo[1,2-a]pyridin-2-yl)pyrimidin-2-yl)morpholine; (R)-4-(5-(1-phenyl-2,3-dihydro-1H-benzo[d]pyrrolo[1,2-a]imidazol-7-yl)pyrimidin-2-yl)morpholine; 7-(5-((S)-4-phenyl-3,4-dihydro-1H-benzo[4,5]imidazo[2,1-c][1,4]oxazin-7-yl)pyrimidin-2-yl)hexahydroimidazo[1,5-a]pyrazin-3 (2H)-one; 7-(5-((S)-4-phenyl-3,4-dihydro-1H-benzo[4,5]imidazo[2,1-c][1,4]oxazin-7-yl)pyrimidin-2-yl)hexahydroimidazo[1,5-a]pyrazin-3 (2H)-one; 4-(5-(8-(2,5-dimethylphenyl)-7,8-dihydro-6H-pyrrolo[2′,1′:2,3]imidazo[4,5-b]pyridin-2-yl)pyrimidin-2-yl)piperazin-2-one; 2-(5-(1-(2,5-dimethylphenyl)-2,3-dihydro-1H-benzo[d]pyrrolo[1,2-a]imidazol-7-yl)pyrimidin-2-yl)propan-2-ol; 3,3-difluoro-1-(5-((S)-4-phenyl-3,4-dihydro-1H-benzo[4,5]imidazo[2,1-c][1,4]oxazin-7-yl)pyrimidin-2-yl)piperidin-4-ol; 7-(5-(4-(3-fluorophenyl)-3,4-dihydro-1H-benzo[4,5]imidazo[2,1-c][1,4]oxazin-7-yl)pyrimidin-2-yl)hexahydroimidazo[1,5-a]pyrazin-3 (2H)-one; (R)-2-(5-(1-(2-methoxyphenyl)-2,3-dihydro-1H-cyclopenta[4,5]imidazo[1,2-a]pyridin-7-yl)pyrimidin-2-yl)propan-2-ol; 7-(4-(isopropylsulfonyl)phenyl)-1-phenyl-2,3-dihydro-1H-benzo[d]pyrrolo[1,2-a]imidazole; 2-(5-(9-(2-Methoxyphenyl)-6,7,8,9-tetrahydrobenzo[4,5]imidazo[1,2-a]pyridin-2-yl)pyrimidin-2-yl)propan-2-ol; 4-(5-(8-(2,5-dimethylphenyl)-7,8-dihydro-6H-pyrrolo[2′,1′:2,3]imidazo[4,5-b]pyridin-2-yl)pyrimidin-2-yl)morpholine; (S)-7-(5-(4-phenyl-3,4-dihydro-1H-benzo[4,5]imidazo[2,1-c][1,4]oxazin-7-yl)pyrimidin-2-yl)-7-azaspiro[3.5]nonan-2-ol; 4-(5-(9-(2-methoxyphenyl)-6,7,8,9-tetrahydrobenzo[4,5]imidazo[1,2-a]pyridin-2-yl)pyrimidin-2-yl)morpholine; 1-(5-(9-phenyl-6,7,8,9-tetrahydroimidazo[1,2-a:5,4-b′]dipyridin-2-yl)pyrimidin-2-yl)piperidin-4-ol; (R)-2-(5-(1-Phenyl-2,3-dihydro-1H-benzo[d]pyrrolo[1,2-a]imidazol-7-yl)pyrimidin-2-yl)propan-2-ol; 2-(5-(10-(2-methoxyphenyl)-7,8,9,10-tetrahydro-6H-cyclohepta[4,5]imidazo[1,2-a]pyridin-2-yl)pyrimidin-2-yl)propan-2-ol; N-methyl-4-(1-phenyl-2,3-dihydro-1H-benzo[d]pyrrolo[1,2-a]imidazol-7-yl)benzenesulfonamide; (S)-7-(2-morpholinopyrimidin-5-yl)-4-phenyl-3,4-dihydro-1H-benzo[4,5]imidazo[2,1-c][1,4]oxazine; (S)-7-(5-(4-phenyl-3,4-dihydro-1H-benzo[4,5]imidazo[2,1-c][1,4]oxazin-7-yl)pyrimidin-2-yl)-7-azaspiro[3.5]nonan-2-ol; 2-(5-(1-(2-methoxyphenyl)-2,3-dihydro-1H-cyclopenta[4,5]imidazo[1,2-a]pyridin-7-yl)pyrimidin-2-yl)propan-2-ol; (R)-2-(5-(1-phenyl-1,2,3,4-tetrahydrobenzo[4,5]imidazo[1,2-a]pyridin-8-yl)pyrimidin-2-yl)propan-2-ol; ethyl 2-[[5-[9-(2-methoxyphenyl)-6,7,8,9-tetrahydropyrido[1,2-a]benzimidazol-2-yl]pyrimidin-2-yl]amino]acetate.r 2-((5-(9-(2-methoxyphenyl)-6,7,8,9-tetrahydrobenzo[4,5]imidazo[1,2-a]pyridin-2-yl)pyridin-2-yl)oxy) acetic acid. In certain embodiments, the compound is selected from the group consisting of: (8aR)-7-(5-(6′,8′-dihydro-2H-spiro[benzofuran-3,9′-pyrido[3′,2′:4,5]imidazo[2,1-c][1,4]oxazin]-2′-yl)pyrimidin-2-yl)hexahydroimidazo[1,5-a]pyrazin-3 (2H)-one; 3-((5-(6′,8′-dihydro-2H-spiro[benzofuran-3,9′-pyrido[3′,2′:4,5]imidazo[2,1-c][1,4]oxazin]-2′-yl)pyrimidin-2-yl)amino)cyclobutanol; 5-(6′,8′-dihydro-2H-spiro[benzofuran-3,9′-pyrido[3′,2′:4,5]imidazo[2,1-c][1,4]oxazin]-2′-yl)-N-(tetrahydrofuran-3-yl)pyrimidin-2-amine; 1-(5-(6′,8′-dihydro-2H-spiro[benzofuran-3,9′-pyrido[3′,2′:4,5]imidazo[2,1-c][1,4]oxazin]-2′-yl)pyrimidin-2-yl)piperidin-3-ol; 2′-(2-(4-methylpiperazin-1-yl)pyrimidin-5-yl)-6′,8′-dihydro-2H-spiro[benzofuran-3,9′-pyrido[3′,2′:4,5]imidazo[2,1-c][1,4]oxazine]; 2′-(2-morpholinopyrimidin-5-yl)-6′,8′-dihydro-2H-spiro[benzofuran-3,9′-pyrido[3′,2′:4,5]imidazo[2,1-c][1,4]oxazine]; 1-(5-(1,2′,3,3′-tetrahydrospiro[benzo[4,5]imidazo[2,1-c][1,4]oxazine-4,1′- inden]-7-yl)pyrimidin-2-yl)piperidin-4-ol; (1-(5-(2,3,6′,8′-tetrahydrospiro[indene-1,9′-pyrido[3′,2′:4,5]imidazo[2,1-c][1,4]oxazin]-2′-yl)pyrimidin-2-yl)piperidin-3-yl) methanol; (8aS)-7-(5-(2,3,6′,8′-tetrahydrospiro[indene-1,9′-pyrido[3′,2′:4,5]imidazo[2,1-c][1,4]oxazin]-2′-yl)pyrimidin-2-yl)hexahydroimidazo[1,5-a]pyrazin-3 (2H)-one; 2′-(2-(1,4-oxazepan-4-yl)pyrimidin-5-yl)-2,3,6′,8′-tetrahydrospiro[indene-1,9′-pyrido[3′,2′:4,5]imidazo[2,1-c][1,4]oxazine]; 3,3-difluoro-1-(5-(2,3,6′,8′-tetrahydrospiro[indene-1,9′-pyrido[3′,2′:4,5]imidazo[2,1-c][1,4]oxazin]-2′-yl)pyrimidin-2-yl)piperidin-4-ol; 2-hydroxy-1-(4-(5-(2,3,6′,8′-tetrahydrospiro[indene-1,9′-pyrido[3′,2′:4,5]imidazo[2,1-c][1,4]oxazin]-2′-yl)pyrimidin-2-yl)piperazin-1-yl)propan-1-one; 2-hydroxy-1-(4-(5-(2,3,6′,8′-tetrahydrospiro[indene-1,9′-pyrido[3′,2′:4,5]imidazo[2,1-c][1,4]oxazin]-2′-yl)pyrimidin-2-yl)piperazin-1-yl)ethanone; (4-fluoro-1-(5-(2,3,6′,8′-tetrahydrospiro[indene-1,9′-pyrido[3′,2′:4,5]imidazo[2,1-c][1,4]oxazin]-2′-yl)pyrimidin-2-2′-(2-morpholinopyrimidin-5-yl)-2,3,6′,8′-tetrahydrospiro[indene-yl)piperidin-4-yl) methanol; 1,9′-pyrido[3′,2′:4,5]imidazo[2,1-c][1,4]oxazine]; 1-(5-(2,3,6′,8′-tetrahydrospiro[indene-1,9′-pyrido[3′,2′:4,5]imidazo[2,1-c][1,4]oxazin]-2′-yl)pyrimidin-2-yl)azepan-4-ol; (S)-2-(5-(6′,8′-dihydrospiro[chroman-4,9′-pyrido[3′,2′:4,5]imidazo[2,1-c][1,4]oxazin]-2′-yl)pyrimidin-2-yl)propan-2-ol; (R)-2-(5-(6′,8′-dihydrospiro[chroman-4,9′-pyrido[3′,2′:4,5]imidazo[2,1-c][1,4]oxazin]-2′-yl)pyrimidin-2-yl)propan-2-ol; (8aR)-7-(5-(6′,8′-dihydro-2H-spiro[benzofuran-c][1,4]oxazin]-2′-yl)pyrimidin-2-yl)hexahydroimidazo[1,5-3,9′-pyrido[3′,2′:4,5]imidazo[2,1-a]pyrazin-3 (2H)-one; 3-((5-(6′,8′-dihydro-2H-spiro[benzofuran-3,9′-pyrido[3′,2′:4,5]imidazo[2,1-c][1,4]oxazin]-2′-yl)pyrimidin-2-yl)amino)cyclobutanol; 5-(6′,8′-dihydro-2H-spiro[benzofuran-3,9′-pyrido[3′,2′:4,5]imidazo[2,1-c][1,4]oxazin]-2′-yl)-N-(tetrahydrofuran-3-yl)pyrimidin-2-amine; 1-(5-(6′,8′-dihydro-2H-spiro[benzofuran-3,9′-pyrido[3′,2′:4,5]imidazo[2,1-c][1,4]oxazin]-2′-yl)pyrimidin-2-yl)piperidin-3-ol; 2′-(2-(4-methylpiperazin-1-yl)pyrimidin-5-yl)-6′,8′-dihydro-2H-spiro[benzofuran-3,9′-pyrido[3′,2′:4,5]imidazo[2,1-c][1,4]oxazine]; 2′-(2-morpholinopyrimidin-5-yl)-6′,8′-dihydro-2H-spiro[benzofuran-3,9′-pyrido[3′,2′:4,5]imidazo[2, 1-c][1,4]oxazine]; 1-(5-(1,2′,3,3′-tetrahydrospiro[benzo[4,5]imidazo[2,1-c][1,4]oxazine-4, 1′-inden]-7-yl)pyrimidin-2-yl)piperidin-4-ol; (1-(5-(2,3,6′,8′-tetrahydrospiro[indene-1,9′-pyrido[3′,2′:4,5]imidazo[2,1-c][1,4]oxazin]-2′-yl)pyrimidin-2-yl)piperidin-3-yl) methanol; (8aR)-7-(5-(2,3,6′,8′-tetrahydrospiro[indene-1,9′-pyrido[3′,2′:4,5]imidazo[2,1-c][1,4]oxazin]-2′-yl)pyrimidin-2-yl)hexahydroimidazo[1,5-a]pyrazin-3 (2H)-one; 2′-(2-(1,4-oxazepan-4-yl)pyrimidin-5-yl)-2,3,6′,8′-tetrahydrospiro[indene-1,9′-pyrido[3′,2′:4,5]imidazo[2,1-c][1,4]oxazine]; pyrido[3′,2′:4,5]imidazo[2,1-c][1,4]oxazin]-2′-yl)pyrimidin-2-yl)piperidin-4-ol; 2-hydroxy-1-(4-(5-(2,3,6′,8′-tetrahydrospiro[indene-1,9′-pyrido[3′,2′:4,5]imidazo[2,1-c][1,4]oxazin]-2′-yl)pyrimidin-2-yl)piperazin-1-yl)propan-1-one; 2-hydroxy-1-(4-(5-(2,3,6′,8′-tetrahydrospiro[indene-1,9′-pyrido[3′,2′:4,5]imidazo[2,1-c][1,4]oxazin]-2′-yl)pyrimidin-2-3,3-difluoro-1-(5-(2,3,6′,8′-tetrahydrospiro[indene-1,9′-yl)piperazin-1-yl)ethanone; (4-fluoro-1-(5-(2,3,6′,8′-tetrahydrospiro[indene-1,9′-pyrido[3′,2′:4,5]imidazo[2,1-c][1,4]oxazin]-2′-yl)pyrimidin-2-yl)piperidin-4-yl) methanol; 2′-(2-morpholinopyrimidin-5-yl)-2,3,6′,8′-tetrahydrospiro[indene-1,9′-pyrido[3′,2′:4,5]imidazo[2, 1-c][1,4]oxazine]; 1-(5-(2,3,6′,8′-tetrahydrospiro[indene-1,9′-pyrido[3′,2′:4,5]imidazo[2,1-c][1,4]oxazin]-2′-yl)pyrimidin-2-yl)azepan-4-ol; (R)-1-((4,4-difluorocyclohexyl)methyl)-4-(8-phenyl-7,8-dihydro-6H-pyrrolo[2′,1′: 2,3]imidazo[4,5-b]pyridin-2-yl)pyridin-2 (1H)-one; (1r,4r)-4-((4-(6′,8′-dihydro-2H-spiro[benzofuran-3,9′-pyrido[3′,2′:4,5]imidazo[2,1-c][1,4]oxazin]-2′-yl)pyridin-2-yl)oxy)cyclohexanol; (1s,4s)-4-((4-(6′,8′-dihydro-2H-spiro[benzofuran-3,9′-pyrido[3′,2′:4,5]imidazo[2,1-c][1,4]oxazin]-2′-yl)pyridin-2-yl)oxy)cyclohexanol; 3-((4-(6′,8′-dihydro-2H-spiro[benzofuran-3,9′-pyrido[3′,2′:4,5]imidazo[2,1-c][1,4]oxazin]-2′-yl)pyridin-2-yl)oxy)cyclopentanol; 2′-(2-morpholinopyrimidin-5-yl)-6′, 7′-dihydrospiro[cyclohexane-1,9′-pyrano[4′,3′:4,5]imidazo[1,2-b]pyridazine]; 2′-(2-morpholinopyrimidin-5-yl)-6′,7′-dihydrospiro[chroman-4,9′-pyrano[4′,3′:4,5]imidazo[1,2-b]pyridazine]; 2′-(2-(piperazin-1-yl)pyrimidin-5-yl)-6′H,8′H-spiro[chromane-4,9′-pyrido[3′,2′:4,5]imidazo[2,1-c][1,4]oxazine]; 2′-(2-methoxypyrimidin-5-yl)-6′,8′-dihydrospiro[chroman-4,9′-pyrido[3,2′:4,5]imidazo[2, 1-c][1,4]oxazine]; 2′-(2-ethoxypyrimidin-5-yl)-6′,8-dihydrospiro[chroman-4,9′-pyrido[3,2′:4,5]imidazo[2,1-c][1,4]oxazine]; 2′-(2-(methylsulfonyl)pyrimidin-5-yl)-6′,8′-dihydrospiro[chroman-4,9′-pyrido[3′,2′:4,5]imidazo[2,1-c][1,4]oxazine]; 2′-(2-(1,4-diazepan-1-yl)pyrimidin-5-yl)-6′,8′-dihydrospiro[chroman-4,9′-pyrido[3′,2′:4,5]imidazo[2,1-c][1,4]oxazine]; 5-(6′,8′-dihydrospiro[chroman-4,9′-pyrido[3′,2′:4,5]imidazo[2,1-c][1,4]oxazin]-2′-yl)-N-isopropylpyrimidin-2-amine; 2′-(2-morpholinopyrimidin-5-yl)-6′,8-dihydrospiro[chroman-4,9′-pyrido[3′,2′:4,5]imidazo[2,1-c][1,4]oxazine]; 2-(5-(6′,8′-dihydrospiro[chroman-4,9′-pyrido[3′,2′:4,5]imidazo[2,1-c][1,4]oxazin]-2′-yl)pyrimidin-2-yl)propan-2-ol; 2′-(2-((tetrahydro-2H-pyran-4-yl)oxy)pyridin-4-yl)-6′,8′-dihydrospiro[chroman-4,9′-pyrido[3′,2′:4,5]imidazo[2, 1-c][1,4]oxazine]; 5-(6′H, 8′H-Spiro [chromane-4,9′-pyrido[3′,2′:4,5]imidazo[2,1-c][1,4]oxazin]-2′-yl)-N-(tetrahydrofuran-3-yl)pyrimidin-2-amine; (8aR)-7-(5-(6′,8′-dihydrospiro[chroman-4,9′-pyrido[3′,2′:4,5]imidazo[2,1-c][1,4]oxazin]-2′-yl)pyrimidin-2-yl)hexahydroimidazo[1,5-a]pyrazin-3 (2H)-one; 1-(5-(6′,8′-dihydrospiro[chroman-4,9′-pyrido[3′,2′:4,5]imidazo[2,1-c][1,4]oxazin]-2′-yl)pyrimidin-2-yl)piperidin-4-ol; 1-(5-(6′,8′-dihydrospiro[chroman-4,9′-pyrido[3′,2′:4,5]imidazo[2,1-c][1,4]oxazin]-2′-yl)pyrimidin-2-yl)piperidin-3-ol; 1-(5-(6′,8′-dihydrospiro[chroman-4,9′-pyrido[3′,2′:4,5]imidazo[2,1-c][1,4]oxazin]-2′-yl)pyrimidin-2-yl)azetidin-3-ol; 1-(5-(6′,8′-dihydrospiro[chroman-4,9′-pyrido[3′,2′:4,5]imidazo[2, 1-c][1,4]oxazin]-2′-yl)pyrimidin-2-yl)pyrrolidin-3-ol; 3-((5-(6′,8′-dihydrospiro[chroman-4,9′-pyrido[3′,2′:4,5]imidazo[2,1-c][1,4]oxazin]-2′-yl)pyrimidin-2-yl)amino)cyclobutanol; 1-(5-(6′,8′-dihydrospiro[chroman-4,9′-pyrido[3′,2′:4,5]imidazo[2,1-c][1,4]oxazin]-2′-yl)pyrimidin-2-yl)-3-methylazetidin-3-ol; 2′-(2-(4-methylpiperazin-1-yl)pyrimidin-5-yl)-6′,8′-dihydrospiro[chroman-4,9′-pyrido[3′,2′:4,5]imidazo[2,1-c][1,4]oxazine]; 2′-(5,5-Dimethyl-2,5-dihydro-1H-pyrrol-3-yl)-2H,6′H,8′H-spiro[benzofuran-3,9′-pyrido[3′,2′:4,5]imidazo[2,1-c][1,4]oxazine]; 2′-(2,5-dihydro-1H-pyrrol-3-yl)-6′,8′-dihydro-2H-spiro[benzofuran-3,9′-pyrido[3′,2′:4,5]imidazo[2,1-c][1,4]oxazine]; 2′-(2-(piperazin-1-yl)pyrimidin-5-yl)-2H,6′H,8′H-spiro[benzofuran-3,9′-pyrido[3′,2′:4,5]imidazo[2,1-c][1,4]oxazine]; 2′-(2-((tetrahydro-2H-pyran-4-yl)oxy)pyridin-4-yl)-6′,8′-dihydro-2H-spiro[benzofuran-3,9′-pyrido[3′,2′:4,5]imidazo[2,1-c][1,4]oxazine]; 2′-(2-methoxypyrimidin-5-yl)-6′,8′-dihydro-2H-spiro[benzofuran-3,9′-pyrido[3′,2′:4,5]imidazo[2,1-c][1,4]oxazine]; 2-(tert-butoxy)-1-((2S)-4-(5-(6′,8′-dihydro-2H-spiro[benzofuran-3,9′-pyrido[3′,2′:4,5]imidazo[2,1-c][1,4]oxazin]-2′-yl)pyrimidin-2-yl)-2-methylpiperazin-1-yl)ethanone; 2-(tert-butoxy)-1-((3S)-4-(5-(6′,8′-dihydro-2H-spiro[benzofuran-3,9′-pyrido[3′,2′:4,5]imidazo[2,1-c][1,4]oxazin]-2′-yl)pyrimidin-2-yl)-3-methylpiperazin-1-yl)ethanone; 2-(tert-butoxy)-1-((3R)-4-(5-(6′,8′-dihydro-2H-spiro[benzofuran-3,9′-pyrido[3′,2′:4,5]imidazo[2,1-c][1,4]oxazin]-2′-yl)pyrimidin-2-yl)-3-methylpiperazin-1-yl)ethanone; 2-(tert-butoxy)-1-((2R)-4-(5-(6′,8′-dihydro-2H-spiro[benzofuran-3,9′-pyrido[3′,2′:4,5]imidazo[2,1-c][1,4]oxazin]-2′-yl)pyrimidin-2-yl)-2-methylpiperazin-1-yl)ethanone; 1-((2S)-4-(5-(2H,6′H,8′H-spiro[benzofuran-3,9′-pyrido[3′,2′:4,5]imidazo[2,1-c][1,4]oxazin]-2′-yl)pyrimidin-2-yl)-2-methylpiperazin-1-yl)-2-hydroxyethan-1-one 1-((3S)-4-(5-(6′,8′-dihydro-2H-spiro[benzofuran-3,9′-pyrido[3′,2′:4,5]imidazo[2,1-c][1,4]oxazin]-2′-yl)pyrimidin-2-yl)-3-methylpiperazin-1-yl)-2-hydroxyethanone; 1-((3R)-4-(5-(6′,8′-dihydro-2H-spiro[benzofuran-3,9′-pyrido[3′,2′:4,5]imidazo[2,1-c][1,4]oxazin]-2′-yl)pyrimidin-2-yl)-3-methylpiperazin-1-yl)-2-hydroxyethanone; 1-((2R)-4-(5-(6′,8′-dihydro-2H-spiro[benzofuran-3,9′-pyrido[3′,2′:4,5]imidazo[2,1-c][1,4]oxazin]-2′-yl)pyrimidin-2-yl)-2-methylpiperazin-1-yl)-2-hydroxyethanone; 2-(5-(2,3-dihydro-6′H, 8′H-spiro[indene-1,9′-pyrido[3′,2′:4,5]imidazo[2,1-c][1,4]oxazin]-2′-yl)pyrimidin-2-yl)propan-2-ol; 2′-(2-(2-hydroxypropan-2-yl)pyrimidin-5-yl)-6′,8′-dihydrospiro[indene-1,9′-pyrido[3′,2′:4,5]imidazo[2,1-c][1,4]oxazin]-3 (2H)-one; 2′-(2-(2-hydroxypropan-2-yl)pyrimidin-5-yl)-2,3-dihydro-6′H,8′H-spiro[indene-1,9′-pyrido[3′,2′:4,5]imidazo[2,1-c][1,4]oxazin]-6′-ol; 2′-(1-(pyrimidin-4-yl)-1,2,3,6-tetrahydropyridin-4-yl)-2H,6′H, 8′H-spiro[benzofuran-3,9′-pyrido[3′,2′:4,5]imidazo[2,1-c][1,4]oxazine]; 1-(4-(6′,8′-dihydro-2H-spiro[benzofuran-3,9′-pyrido[3′,2′:4,5]imidazo[2,1-c][1,4]oxazin]-2′-yl)-5,6-dihydropyridin-1 (2H)-yl)-3-methoxy-3-methylbutan-1-one; (S)-1-(5-(6′,8′-dihydro-2H-spiro[benzofuran-3,9′-pyrido[3′,2′:4,5]imidazo[2,1-c][1,4]oxazin]-2′-yl)pyrimidin-2-yl)piperidin-4-ol; (R)-7-(5-((S)-6′,8′-dihydro-2H-spiro[benzofuran-3,9′-pyrido[3′,2′:4,5]imidazo[2, 1-c][1,4]oxazin]-2′-yl)pyrimidin-2-yl)hexahydroimidazo[1,5-(1S,4r)-4-((4-((S)-6′,8′-Dihydro-2H-spiro[benzofuran-3,9′-a]pyrazin-3 (2H)-one; pyrido[3′,2′:4,5]imidazo[2,1-c][1,4]oxazin]-2′-yl)pyridin-2-yl)oxy)cyclohexanol; 1-((S)-4-(5-((S)-6′,8′-dihydro-2H-spiro[benzofuran-3,9′-pyrido[3′,2′:4,5]imidazo[2,1-c][1,4]oxazin]-2′-yl)pyrimidin-2-yl)-2-methylpiperazin-1-yl)-2-hydroxyethanone; 1-((R)-4-(5-((S)-6′,8′-dihydro-2H-spiro[benzofuran-3,9′-pyrido[3′,2′:4,5]imidazo[2,1-c][1,4]oxazin]-2′-yl)pyrimidin-2-yl)-2-methylpiperazin-1-yl)-2-hydroxyethanone; 1-((R)-4-(5-((R)-6′,8′-dihydro-2H-spiro[benzofuran-3,9′-pyrido[3′,2′:4,5]imidazo[2,1-c][1,4]oxazin]-2′-yl)pyrimidin-2-yl)-2-methylpiperazin-1-yl)-2-hydroxyethanone; 1-((S)-4-(5-((R)-6′,8′-dihydro-2H-spiro[benzofuran-3,9′-pyrido[3′,2′:4,5]imidazo[2,1-c][1,4]oxazin]-2′-yl)pyrimidin-2-yl)-2-methylpiperazin-1-yl)-2-hydroxyethanone; 1-(5-(6′,8′-dihydrospiro[isochroman-4,9′-pyrido[3′,2′:4,5]imidazo[2, 1-c][1,4]oxazin]-2′-yl)pyrimidin-2-yl)piperidin-4-ol; (8aR)-7-(5-(6′,8′-dihydrospiro[isochroman-4,9′-pyrido[3′,2′:4,5]imidazo[2,1-c][1,4]oxazin]-2′-yl)pyrimidin-2-yl)hexahydroimidazo[1,5-a]pyrazin-3 (2H)-one; 2′-(2-morpholinopyrimidin-5-yl)-6′,8′-dihydrospiro[isochroman-4,9′-pyrido[3′,2′:4,5]imidazo[2,1-c][1,4]oxazine]; 2-(5-(2,3-dihydro-6′H,8′H-spiro[indene-1,9′-pyrido[3′,2′:4,5]imidazo[2,1-c][1,4]oxazin]-2′-yl)pyrimidin-2-yl)propan-2-amine; (S)-2-(5-(6′,8′-dihydro-2H-spiro[benzofuran-3,9′-pyrido[3′,2′:4,5]imidazo[2,1-c][1,4]oxazin]-2′-yl)pyrimidin-2-yl)propan-2-amine; or 2′-(2-(2-hydroxypropan-2-yl)pyrimidin-5-yl)-2,3,6′,8′-tetrahydrospiro[indene-1,9′-pyrido[3′,2′:4,5]imidazo[2,1-c][1,4]oxazin]-3-ol. In certain embodiments, the compound is selected from the group consisting of: ((R)-1-(5-((R)-8-phenyl-7,8-dihydro-6H-pyrrolo[2′,1′:2,3]imidazo[4,5-b]pyridin-2-yl)pyrimidin-2-yl)pyrrolidin-2-yl) methanol; ((S)-1-(5-((R)-8-phenyl-7,8-dihydro-6H-pyrrolo[2′,11:2,3]imidazo[4,5-b]pyridin-2-yl)pyrimidin-2-yl)pyrrolidin-2-yl) methanol; (R)-1-(2-(methylsulfonyl)ethyl)-4-(8-phenyl-7,8-dihydro-6H-pyrrolo[2′,1′:2,3]imidazo[4,5-b]pyridin-2-yl)pyridin-2 (1H)-one; (R)-1-(2-hydroxy-2-methylpropyl)-4-(8-phenyl-7,8-dihydro-6H-pyrrolo[2′,1′:2,3]imidazo[4,5-b]pyridin-2-yl)pyridin-2 (1H)-one; 1-(5-((R)-8-phenyl-7,8-dihydro-6H-pyrrolo[2′,1′:2,3]imidazo[4,5-b]pyridin-2-yl)pyrimidin-2-yl)ethanol; 2-cyclopropyl-1-(5-((R)-8-phenyl-7,8-dihydro-6H-pyrrolo[2′,1′:2,3]imidazo[4,5-b]pyridin-2-yl)pyrimidin-2-yl)ethanol; (5-((R)-8-phenyl-7,8-dihydro-6H-pyrrolo[2′,1′:2,3]imidazo[4,5-b]pyridin-2-yl)pyrimidin-2-yl) (tetrahydro-2H-pyran-4-yl) methanol; 1-(5-((R)-8-phenyl-7,8-dihydro-6H-pyrrolo[2′,1′:2,3]imidazo[4,5-b]pyridin-2-yl)pyrimidin-2-yl)-1-(tetrahydro-2H-pyran-4-yl)ethanol; 1-cyclopropyl-2-(5-((R)-8-phenyl-7,8-dihydro-6H-pyrrolo[2′,1′:2,3]imidazo[4,5-b]pyridin-2-yl)pyrimidin-2-yl)propan-2-ol; 1-((R)-2-methyl-4-(5-((R)-8-phenyl-7,8-dihydro-6H-pyrrolo[2′,1′:2,3]imidazo[4,5-b]pyridin-2-yl)pyrimidin-2-yl)piperazin-1-yl)ethanone; 1-((S)-2-methyl-4-(5-((R)-8-phenyl-7,8-dihydro-6H-pyrrolo[2′,1′:2,3]imidazo[4,5-b]pyridin-2-yl)pyrimidin-2-yl)piperazin-1-yl)ethanone; (1R,3R)-3-((4-((R)-8-phenyl-7,8-dihydro-6H-pyrido[3,2-b]pyrrolizin-2-yl)pyridin-2-yl)oxy)cyclopentanecarbonitrile; (R)-1-((4,4-difluorocyclohexyl)methyl)-4-(8-phenyl-7,8-dihydro-6H-pyrrolo[2′,1′:2,3]imidazo[4,5-b]pyridin-2-yl)pyridin-2 (1H)-one; 2-(5-(8-(pyridin-2-yl)-7,8-dihydro-6H-pyrrolo[2′,1′:2,3]imidazo[4,5-b]pyridin-2-yl)pyrimidin-2-yl)propan-2-ol; 1-((S)-2-methyl-4-(5-((R)-8-phenyl-7,8-dihydro-6H-pyrrolo[2′,1′:2,3]imidazo[4,5-b]pyridin-2-yl)pyrimidin-2-yl)piperazin-1-yl)ethanone; 2-hydroxy-1-((R)-2-methyl-4-(5-((R)-8-phenyl-7,8-dihydro-6H-pyrrolo[2′,1′:2,3]imidazo[4,5-b]pyridin-2-yl)pyrimidin-2-yl)piperazin-1-yl)ethanone; (R)-7-(5-((R)-8-(3-methoxyphenyl)-7,8-dihydro-6H-pyrrolo[2′,1′:2,3]imidazo[4,5-b]pyridin-2-yl)pyrimidin-2-yl)hexahydroimidazo[1,5-a]pyrazin-3 (2H)-one; 1-((R)-4-(5-((R)-8-(3-methoxyphenyl)-7,8-dihydro-6H-pyrrolo[2′,1′:2,3]imidazo[4,5-b]pyridin-2-yl)pyrimidin-2-yl)-2-methylpiperazin-1-yl)ethanone; (R)-7-(5-((S)-8-(3-methoxyphenyl)-7,8-dihydro-6H-pyrrolo[2′,1′:2,3]imidazo[4,5-b]pyridin-2-yl)pyrimidin-2-yl)hexahydroimidazo[1,5-a]pyrazin-3 (2H)-one; (R)-7-(5-((R)-8-(2-methoxyphenyl)-7,8-dihydro-6H-pyrrolo[2′,1′:2,3]imidazo[4,5-b]pyridin-2-yl)pyrimidin-2-yl)hexahydroimidazo[1,5-a]pyrazin-3 (2H)-one; (S)-7-(5-((R)-8-(2-methoxyphenyl)-7,8-dihydro-6H-pyrrolo[2′,1′:2,3]imidazo[4,5-b]pyridin-2-yl)pyrimidin-2-yl)hexahydroimidazo[1,5-a]pyrazin-3 (2H)-one; (R)-1-(5-(8-(2-methoxyphenyl)-7,8-dihydro-6H-pyrrolo[2′,11:2,3]imidazo[4,5-b]pyridin-2-yl)pyrimidin-2-yl)piperidin-4-ol; 2-hydroxy-1-((R)-4-pyrrolo[2′,1′:2,3]imidazo[4,5-b]pyridin-2-(5-((R)-8-(2-methoxyphenyl)-7,8-dihydro-6H-yl)pyrimidin-2-yl)-2-methylpiperazin-1-yl)ethanone; (R)-7-(5-((S)-8-(2-methoxyphenyl)-7,8-dihydro-6H-pyrrolo[2′,1′:2,3]imidazo[4,5-b]pyridin-2-yl)pyrimidin-2-yl)hexahydroimidazo[1,5-a]pyrazin-3 (2H)-one; 1-((R)-4-(5-((R)-8-(3-(Hydroxymethyl)phenyl)-7,8-dihydro-6H-pyrrolo[2′,1′:2,3]imidazo[4,5-b]pyridin-2-yl)pyrimidin-2-yl)-2-methylpiperazin-1-yl)ethanone; 1-((R)-4-(5-((R)-8-(3-(Hydroxymethyl)phenyl)-7,8-dihydro-6H-pyrrolo[2′,1′:2,3]imidazo[4,5-b]pyridin-2-yl)pyrimidin-2-yl)-2-methylpiperazin-1-yl)ethanone; (S)-1-(5-((R)-8-phenyl-7,8-dihydro-6H-pyrrolo[2′,1′:2,3]imidazo[4,5-b]pyridin-2-yl)pyrimidin-2-yl)piperidin-4-yl 2-amino-3-methylbutanoate; (R)-1-(5-(8-phenyl-7,8-dihydro-6H-pyrrolo[2′,1′:2,3]imidazo[4,5-b]pyridin-2-yl)pyrimidin-2-yl)piperidin-4-yl dihydrogen phosphate hydrochloride; (R)-8-phenyl-2-(2-((tetrahydro-2H-pyran-4-yl)oxy)pyridin-4-yl)-7,8-dihydro-6H-pyrrolo[2′,11:2,3]imidazo[4,5-b]pyridine; 3-((4-((R)-8-phenyl-7,8-dihydro-6H-pyrrolo[2′,1:2,3]imidazo[4,5-b]pyridin-2-yl)pyridin-2-yl)oxy)cyclopentanol; (R)-4-((4-(8-phenyl-7,8-dihydro-6H-pyrrolo[2′,1:2,3]imidazo[4,5-b]pyridin-2-yl)pyridin-2-yl)oxy)cyclohexanol; (R)-2-(2-(oxetan-3-yloxy)pyridin-4-yl)-8-phenyl-7,8-dihydro-6H-pyrrolo[2′,1′:2,3]imidazo[4,5-b]pyridine; 3-((4-((R)-8-phenyl-7,8-dihydro-6H-pyrrolo[2′,1:2,3]imidazo[4,5-b]pyridin-2-yl)pyridin-2-yl)oxy)cyclohexanol; (R)-2-(2-(oxetan-3-ylmethoxy)pyridin-4-yl)-8-phenyl-7,8-dihydro-6H-pyrrolo[2′,1′:2,3]imidazo[4,5-b]pyridine; (R)-2-(2-(((R)-1-methylpyrrolidin-3-yl)oxy)pyridin-4-yl)-8-phenyl-7,8-dihydro-6H-pyrrolo[2′,1′:2,3]imidazo[4,5-b]pyridine; (R)-2-((4-(8-phenyl-7,8-dihydro-6H-pyrrolo[2′,1:2,3]imidazo[4,5-b]pyridin-2-yl)pyridin-2-yl)oxy) ethanol; (R)-2-(2-(((S)-1-methylpyrrolidin-3-yl)oxy)pyridin-4-yl)-8-phenyl-7,8-dihydro-6H-pyrrolo[2′,1′:2,3]imidazo[4,5-b]pyridine; (1S,4s)-4-((4-((R)-8-phenyl-7,8-dihydro-6H-pyrrolo[2′,1′:2,3]imidazo[4,5-b]pyridin-2-yl)pyridin-2-yl)oxy)cyclohexanol; (8R)-8-phenyl-2-(2-((tetrahydro-2H-pyran-3-yl)oxy)pyridin-4-yl)-7,8-dihydro-6H-pyrrolo[2′,1′:2,3]imidazo[4,5-b]pyridine; (8R)-8-phenyl-2-(2-((tetrahydrofuran-3-yl)oxy)pyridin-4-yl)-7,8-dihydro-6H-pyrrolo[2′,1′:2,3]imidazo[4,5-b]pyridine; (R)-2-(2-(cyclopentyloxy)pyridin-4-yl)-8-phenyl-7,8-dihydro-6H-pyrrolo[2′,1′:2,3]imidazo[4,5-b]pyridine; (R)-2-(2-(cyclohexyloxy)pyridin-4-yl)-8-phenyl-7,8-dihydro-6H-pyrrolo[2′,1′:2,3]imidazo[4,5-b]pyridine; (R)-methyl 4-((4-(8-phenyl-7,8-dihydro-6H-pyrrolo[2′,1′:2,3]imidazo[4,5-b]pyridin-2-yl)pyridin-2-yl)oxy)cyclohexanecarboxylate; methyl 3-((4-((R)-8-phenyl-7,8-dihydro-6H-pyrrolo[2′,1′:2,3]imidazo[4,5-b]pyridin-2-yl)pyridin-2-yl)oxy)cyclopentanecarboxylate; (R)-2-(2-butoxypyridin-4-yl)-8-phenyl-7,8-dihydro-6H-pyrido[3,2-b]pyrrolizine; (1R,3R)-3-((4-((R)-8-phenyl-7,8-dihydro-6H-pyrido[3,2-b]pyrrolizin-2-yl)pyridin-2-yl)oxy)cyclopentanecarbonitrile; (R)-8-phenyl-2-(2-((S)-pyrrolidin-3-yloxy)pyridin-4-yl)-7,8-dihydro-6H-(8R)-8-phenyl-2-(2-(piperidin-3-yloxy)pyridin-4-yl)-pyrrolo[2′,1′:2,3]imidazo[4,5-b]pyridine; 7,8-dihydro-6H-pyrrolo[2′,1′:2,3]imidazo[4,5-b]pyridine; (R)-8-phenyl-2-(2-((R)-pyrrolidin-3-yloxy)pyridin-4-yl)-7,8-dihydro-6H-pyrrolo[2′,1′:2,3]imidazo[4,5-b]pyridine; (8R)-2-(2-(6-azaspiro[3.4]octan-1-yloxy)pyridin-4-yl)-8-phenyl-7,8-dihydro-6H-pyrrolo[2′,1′:2,3]imidazo[4,5-b]pyridine; (R)-2-(2-(6-azaspiro[3.4]octan-2-yloxy)pyridin-4-yl)-8-phenyl-7,8-dihydro-6H-pyrrolo[2′,1′:2,3]imidazo[4,5-b]pyridine; (8R)-2-(2-(6-azaspiro[3.5]nonan-1-yloxy)pyridin-4-yl)-8-phenyl-7,8-dihydro-6H-pyrrolo[2′,1′:2,3]imidazo[4,5-b]pyridine; 1-(5-(6′,8′-sihydro-2H-spiro[benzofuran-3,9′-pyrido[3′,2′:4,5]imidazo[2,1-c][1,4]oxazin]-2′-yl)pyrimidin-2-yl)piperidin-4-ol; (R)-1-((4,4-difluorocyclohexyl)methyl)-4-(8-phenyl-7,8-dihydro-6H-pyrrolo[2′,1′:2,3]imidazo[4,5-b]pyridin-2-yl)pyridin-2 (1H)-one; (R)-1-(2-methoxyethyl)-4-(8-phenyl-7,8-dihydro-6H-pyrrolo[2′,1′:2,3]imidazo[4,5-b]pyridin-2-yl)pyridin-2 (1H)-one; (R)-4-(8-phenyl-7,8-dihydro-6H-pyrrolo[2′,1′:2,3]imidazo[4,5-b]pyridin-2-yl)-1-((tetrahydro-2H-pyran-4-yl)methyl)pyridin-4-((R)-8-phenyl-7,8-dihydro-6H-pyrrolo[2′,1′:2,3]imidazo[4,5-b]pyridin-2-yl)-1-2 (1H)-one; (tetrahydrofuran-3-yl)pyridin-2 (1H)-one; (R)-4-(8-phenyl-7,8-dihydro-6H-pyrrolo[2′,1′:2,3]imidazo[4,5-b]pyridin-2-yl)-1-(tetrahydro-2H-pyran-4-yl)pyridin-2 (1H)-one; (R)-8-phenyl-2-(1-(tetrahydro-2H-pyran-4-yl)-1H-pyrazol-4-yl)-7,8-dihydro-6H-pyrrolo[2′,1′:2,3]imidazo[4,5-b]pyridine; 4-((4-(6′, 8′-dihydro-2H-spiro[benzofuran-3,9′-pyrido[3′,2′:4,5]imidazo[2,1-c][1,4]oxazin]-2′-yl)pyridin-2-yl)oxy)cyclohexanol; (R)-1-(5-(3-fluoro-8-phenyl-7,8-dihydro-6H-pyrrolo[2′,1′:2,3]imidazo[4,5-b]pyridin-2-yl)pyrimidin-2-yl)piperidin-4-ol; 2-(2-morpholinopyrimidin-5-yl)-9-phenyl-7,9-dihydro-6H-pyran [4′,3′:4,5]imidazo[1,2-b]pyridazine; trans-4-((4-(4-(2-methoxyphenyl)-3,4-dihydro-2H-pyran [2′,3′:4,5]imidazo[1,2-a]pyridin-7-yl)pyridin-2-yl)oxy)cyclohexanol; (8aS)-7-(5-(9-phenyl-8,9-dihydro-6H-pyrido[3′,2′:4,5]imidazo[2,1-c][1,4]oxazin-2-yl)pyrimidin-2-yl)hexahydroimidazo[1,5-a]pyrazin-3 (2H)-one; 3,3-difluoro-1-(5-(9-phenyl-8,9-dihydro-6H-pyrido[3′,2′:4,5]imidazo[2,1-c][1,4]oxazin-2-yl)pyrimidin-2-yl)piperidin-4-ol; 1-(5-(9-phenyl-8,9-dihydro-6H-pyrido[3′,2′:4,5]imidazo[2,1-c][1,4]oxazin-2-yl)pyrimidin-2-yl)piperidin-4-ol; 2-(2-(1,4-oxazepan-4-yl)pyrimidin-5-yl)-9-phenyl-8,9-dihydro-6H-pyrido[3′,2′:4,5]imidazo[2, 1-c][1,4]oxazine; (1-(5-(9-phenyl-8,9-dihydro-6H-pyrido[3′,2′:4,5]imidazo[2,1-c][1,4]oxazin-2-yl)pyrimidin-2-yl)piperidin-3-yl) methanol; (4-fluoro-1-(5-(9-phenyl-8,9-dihydro-6H-pyrido[3′,2′:4,5]imidazo[2,1-c][1,4]oxazin-2-yl)pyrimidin-2-yl)piperidin-4-yl) methanol; 2-hydroxy-1-(4-(5-(9-phenyl-8,9-dihydro-6H-pyrido[3′,2′:4,5]imidazo[2,1-c][1,4]oxazin-2-yl)pyrimidin-2-yl)piperazin-1-yl)propan-1-one; 2-hydroxy-1-(4-(5-(9-phenyl-8,9-dihydro-6H-pyrido[3′,2′:4,5]imidazo[2,1-c][1,4]oxazin-2-yl)pyrimidin-2-yl)piperazin-1-yl)ethanone; (R)-8-phenyl-2-(2-((tetrahydro-2H-pyran-4-yl) methoxy)pyridin-4-yl)-7,8-dihydro-6H-pyrrolo[2′,1′:2,3]imidazo[4,5-b]pyridine; (R)-2-(2-(2-methoxyethoxy)pyridin-4-yl)-8-phenyl-7,8-dihydro-6H-pyrrolo[2′,1′:2,3]imidazo[4,5-b]pyridine; (R)-2-methyl-1-((4-(8-phenyl-7,8-dihydro-6H-pyrrolo[2′,1′:2,3]imidazo[4,5-b]pyridin-2-yl)pyridin-2-yl)oxy) propan-2-ol; (R)-8-phenyl-2-(1,2,3,6-tetrahydropyridin-4-yl)-7,8-dihydro-6H-pyrrolo[2′,1′:2,3]imidazo[4,5-b]pyridine; (1S,4s)-4-(((4-((R)-8-phenyl-7,8-dihydro-6H-pyrrolo[2′,1′:2,3]imidazo[4,5-b]pyridin-2-yl)pyridin-2-yl)oxy)methyl)cyclohexanol; (1R,4r)-4-(((4-((R)-8-phenyl-7,8-dihydro-6H-pyrrolo [1′:22′,1:2,3]imidazo[4,5-b]pyridin-2-yl)pyridin-2-yl)oxy)methyl)cyclohexanol; ((1R,4r)-4-(((4-((R)-8-phenyl-7,8-dihydro-6H-pyrrolo[2′,1′:2,3]imidazo[4,5-b]pyridin-2-yl)pyridin-2-yl)oxy)methyl)cyclohexyl) methanol; (R)-8-Phenyl-2-(1-(pyrimidin-4-yl)-1,2,3,6-tetrahydropyridin-4-yl)-7,8-dihydro-6H-pyrrolo[2′,1′:2,3]imidazo[4,5-b]pyridine; methyl 2-(4-((R)-8-phenyl-7,8-dihydro-6H-pyrrolo[2′,1′:2,3]imidazo[4,5-b]pyridin-2-yl)cyclohex-3-en-1-yl)acetate; 1-(5-(8-methyl-8-phenyl-7,8-dihydro-6H-pyrrolo[2′,1′:2,3]imidazo[4,5-b]pyridin-2-yl)pyrimidin-2-yl)piperidin-4-ol; (S)-1-(5-(8-methyl-8-phenyl-7,8-dihydro-6H-pyrrolo[2′,1′:2,3]imidazo[4,5-b]pyridin-2-yl)pyrimidin-2-yl)piperidin-4-ol; (R)-1-(5-(8-methyl-8-phenyl-7,8-dihydro-6H-pyrrolo[2′,1′:2,3]imidazo[4,5-b]pyridin-2-yl)pyrimidin-2-yl)piperidin-4-ol; (S)-2-(5-(8-methyl-8-phenyl-7,8-dihydro-6H-pyrrolo[2′,1′:2,3]imidazo[4,5-b]pyridin-2-yl)pyrimidin-2-yl)propan-2-ol; (R)-2-(5-(8-methyl-8-phenyl-7,8-dihydro-6H-pyrrolo[2′,1′:2,3]imidazo[4,5-b]pyridin-2-yl)pyrimidin-2-yl)propan-2-ol; (R)-2-(5-(8-phenyl-7,8-dihydro-6H-pyrrolo[2′,1′:2,3]imidazo[4,5-b]pyridin-2-yl)pyrimidin-2-yl)propan-2-amine; 2-(2-(4,4-difluoropiperidin-1-yl)pyrimidin-5-yl)-9-phenyl-8,9-dihydro-6H-pyrido-[3′,2′:4,5]imidazo[2,1-c][1,4]oxazine; or (1R,4R)-4-((4-((R)-8-phenyl-7,8-dihydro-6H-pyrrolo[2′,1′:2, 3]imidazo[4,5-b]pyridin-2-yl)pyridin-2-yl)oxy)cyclohexanol. In certain embodiments, the compound is selected from the group consisting of:2-(5-(9-phenyl-6,7,8,9-tetrahydro-6,8-methanoimidazo[1,2-a:5,4-b′]dipyridin-2-yl)pyrimidin-2-yl)propan-2-ol; 9-phenyl-2-(2-((tetrahydro-2H-pyran-4-yl)oxy)pyridin-4-yl)-6,7,8,9-tetrahydro-6,8-methanoimidazo[1,2-a:5,4-b′]dipyridine; 4-(5-(9-phenyl-6,7,8,9-tetrahydro-6,8-methanoimidazo[1,2-a:5,4-b′]dipyridin-2-yl)pyrimidin-2-yl)morpholine; 1-(5-(9-phenyl-6,7,8,9-tetrahydro-6,8-methanoimidazo[1,2-a:5,4-b′]dipyridin-2-yl)pyrimidin-2-yl)piperidin-4-ol; 2-(5-((6R,8S,9S)-9-phenyl-6,7,8,9-tetrahydro-6,8-epoxyimidazo[1,2-a:5,4-b′]dipyridin-2-yl)pyrimidin-2-yl)propan-2-ol; 1-(5-((6R,8S,9S)-9-phenyl-6,7,8,9-tetrahydro-6,8-epoxyimidazo[1,2-a:5,4-b′]dipyridin-2-yl)pyrimidin-2-yl)piperidin-4-ol; 2-(5-(9-phenyl-6,7,8,9-tetrahydro-6,8-methanoimidazo[1,2-a:5,4-b′]dipyridin-2-yl)pyrimidin-2-yl)propan-2-ol; 9-phenyl-2-(2-((tetrahydro-2H-pyran-4-yl)oxy)pyridin-4-yl)-6,7,8,9-tetrahydro-6,8-methanoimidazo[1,2-a:5,4-b′]dipyridine; 1-(5-(9-phenyl-6,7,8,9-tetrahydro-6,8-yl)pyrimidin-2-yl)piperidin-4-ol; 4-(5-(9-phenyl-methanoimidazo[1,2-a:5,4-b′]dipyridin-2-6,7,8,9-tetrahydro-6,8-methanoimidazo[1,2-a:5,4-b′]dipyridin-2-yl)pyrimidin-2-yl)morpholine; 2-(5-((6R,8S,9S)-9-phenyl-6,7,8,9-tetrahydro-6,8-epoxyimidazo[1,2-a:5,4-b′]dipyridin-2-yl)pyrimidin-2-yl)propan-2-ol; or 1-(5-((6R,8S,9S)-9-phenyl-6,7,8,9-tetrahydro-6,8-epoxyimidazo[1,2-a:5,4-b′]dipyridin-2-yl)pyrimidin-2-yl)piperidin-4-ol. The disclosures of U.S. Pat. No 10,266,532 B2 , 9,856,253 B2, and U.S. Patent Applications No. US20160304517A1 and US2018/0179198 A1, are incorporated herein in their entireties by reference.

In certain embodiments, the TNF-alpha Targeting Ligand is selected from the group consisting of:3-(2-(Difluoromethoxy)phenyl)-6-(2-morpholinopyrimidin-5-yl)-2,3-dihydropyrazolo[1,2-a]indazol-9 (1H)-one; (R)-3-(2-(difluoromethoxy)phenyl)-6-(2-morpholinopyrimidin-5-yl)-2,3-dihydropyrazolo[1,2-a]indazol-9 (1H)-one; (S)-3-(2-(difluoromethoxy)phenyl)-6-(2-morpholinopyrimidin-5-yl)-2,3-dihydropyrazolo[1,2-a]indazol-9 (1H)-one; 1-(5-(3-(2-(difluoromethoxy)phenyl)-9-oxo-1,2,3,9-tetrahydropyrazolo[1,2-a]indazol-6-yl)pyrimidin-2-yl)piperidine-4-carboxylic acid; (S)-3-(2-(difluoromethoxy)phenyl)-6-(2-((R)-2-(methoxymethyl)pyrrolidin-1-yl)pyrimidin-5-yl)-2,3-dihydropyrazolo[1,2-a]indazol-9 (1H)-one; (R)-3-(2-(difluoromethoxy)phenyl)-6-(2-((R)-2-(methoxymethyl)pyrrolidin-1-yl)pyrimidin-5-yl)-2,3-dihydropyrazolo[1,2-a]indazol-9 (1H)-one; (R)-3-(2-(difluoromethoxy)phenyl)-6-(2-(((R)-tetrahydrofuran-3-yl)amino)pyrimidin-5-yl)-2,3-dihydropyrazolo[1,2-a]indazol-9 (1H)-one; (S)-3-(2-(difluoromethoxy)phenyl)-6-(2-(((R)-tetrahydrofuran-3-yl)amino)pyrimidin-5-yl)-2,3-dihydropyrazolo[1,2-a]indazol-9 (1H)-one; (R)-3-(2-(difluoromethoxy)phenyl)-6-(2-((R)-2-(hydroxymethyl)morpholino)pyrimidin-5-yl)-2,3-dihydropyrazolo[1,2-a]indazol-9 (1H)-one; (S)-3-(2-(difluoromethoxy)phenyl)-6-(2-((R)-2-(hydroxymethyl)morpholino)pyrimidin-5-yl)-2,3-dihydropyrazolo[1,2-a]indazol-9 (1H)-one; (S)-3-(2-(difluoromethoxy)phenyl)-6-(2-((S)-2-(hydroxymethyl)morpholino)pyrimidin-5-yl)-2,3-dihydropyrazolo[1,2-a]indazol-9 (1H)-one; (R)-3-(2-(difluoromethoxy)phenyl)-6-(2-((S)-2-(hydroxymethyl)morpholino)pyrimidin-5-yl)-2,3-dihydropyrazolo[1,2-a]indazol-9 (1H)-one; (R)-3-(2-(Difluoromethoxy)phenyl)-6-(2-(2-hydroxypropan-2-yl)pyrimidin-5-yl)-2,3-dihydropyrazolo[1,2-a]indazol-9 (1H)-one; (S)-3-(2-(difluoromethoxy)phenyl)-6-(2-(2-hydroxypropan-2-yl)pyrimidin-5-yl)-2,3-dihydropyrazolo[1,2-a]indazol-9 (1H)-one; 3-(2-(difluoromethoxy)phenyl)-6-(2-(4-hydroxypiperidin-1-yl)pyrimidin-5-yl)-2,3-dihydropyrazolo[1,2-a]indazol-9 (1H)-one; (R)-3-(2-(difluoromethoxy)phenyl)-6-(2-((((S)-5-oxopyrrolidin-3-yl)methyl)amino)pyrimidin-5-yl)-2,3-dihydropyrazolo[1,2-a]indazol-9 (1H)-one; (S)-3-(2-(difluoromethoxy)phenyl)-6-(2-((((S)-5-oxopyrrolidin-3-yl)methyl)amino)pyrimidin-5-yl)-2,3-dihydropyrazolo[1,2-a]indazol-9 (1H)-one; (R)-3-(2-(difluoromethoxy)phenyl)-6-(2-((((R)-5-oxopyrrolidin-3-yl)methyl)amino)pyrimidin-5-yl)-2,3-dihydropyrazolo[1,2-a]indazol-9 (1H)-one; (S)-3-(2-(difluoromethoxy)phenyl)-6-(2-((((R)-5-oxopyrrolidin-3-yl)methyl)amino)pyrimidin-5-yl)-2,3-dihydropyrazolo[1,2-a]indazol-9 (1H)-one; (R)-3-(2-(difluoromethoxy)phenyl)-6-(2-(2-hydroxy-7-azaspiro[3.5]nonan-7-yl)pyrimidin-5-yl)-2,3-dihydropyrazolo[1,2-a]indazol-9 (1H)-one; (S)-3-(2-(difluoromethoxy)phenyl)-6-(2-(2-hydroxy-7-azaspiro[3.5]nonan-7-yl)pyrimidin-5-yl)-2,3-dihydropyrazolo[1,2-a]indazol-9 (1H)-one; (S)-3-(2-(difluoromethoxy)phenyl)-6-(2-((S)-3-oxohexahydroimidazo[1,5-a]pyrazin-7 (1H)-yl)pyrimidin-5-yl)-2,3-dihydropyrazolo[1,2-a]indazol-9 (1H)-one; (S)-3-(2-(difluoromethoxy)phenyl)-6-(2-((R)-3-oxohexahydroimidazo[1,5-a]pyrazin-7 (1H)-yl)pyrimidin-5-yl)-2,3-dihydropyrazolo[1,2-a]indazol-9 (1H)-one; (R)-3-(2-(difluoromethoxy)phenyl)-6-(2-((S)-3-oxohexahydroimidazo[1,5-a]pyrazin-7 (1H)-yl)pyrimidin-5-yl)-2,3-dihydropyrazolo[1,2-a]indazol-9 (1H)-one; (R)-3-(2-(difluoromethoxy)phenyl)-6-(2-((R)-3-oxohexahydroimidazo[1,5-a]pyrazin-7 (1H)-yl)pyrimidin-5-yl)-2,3-dihydropyrazolo[1,2-a]indazol-9 (1H)-one; (R)-3-(2-(Difluoromethoxy)phenyl)-6-(2-((3-methoxypropyl)amino)pyrimidin-5-yl)-2,3-dihydropyrazolo[1,2-a]indazol-9 (1H)-one; (S)-3-(2-(Difluoromethoxy)phenyl)-6-(2-((3-methoxypropyl)amino)pyrimidin-5-yl)-2,3-dihydropyrazolo[1,2-a]indazol-9 (1H)-one; (S)-6-(2-(4-Acetylpiperazin-1-yl)pyrimidin-5-yl)-3-(2-(difluoromethoxy)phenyl)-2,3-dihydropyrazolo[1,2-a]indazol-9 (1H)-one; (R)-6-(2-(4-Acetylpiperazin-1-yl)pyrimidin-5-yl)-3-(2-(difluoromethoxy)phenyl)-2,3-dihydropyrazolo[1,2-a]indazol-9 (1H)-one; 6-(2-(difluoromethoxy)phenyl)-3-(2-morpholinopyrimidin-5-yl)-8,9-dihydro-6H-pyridazino [1,2-a]indazol-11 (7H)-one; 6-(2-(difluoromethoxy)phenyl)-3-(2-(4-hydroxypiperidin-1-yl)pyrimidin-5-yl)-8,9-dihydro-6H-pyridazino [1,2-a]indazol-11 (7H)-one; 6-(2-(difluoromethoxy)phenyl)-3-(2-(1,1-dioxidothiomorpholino)pyrimidin-5-yl)-8,9-dihydro-6H-pyridazino [1,2-a]indazol-11 (7H)-one; 3-(2-methoxyphenyl)-6-(2-morpholinopyrimidin-5-yl)-2,3-dihydropyrazolo[1,2-a]indazol-9 (1H)-one; (R)-6-(2-(2-hydroxypropan-2-yl)pyrimidin-5-yl)-3-(2-methoxyphenyl)-2,3-dihydropyrazolo[1,2-a]indazol-9 (1H)-one; (S)-6-(2-(2-hydroxypropan-2-yl)pyrimidin-5-yl)-3-(2-methoxyphenyl)-2,3-dihydropyrazolo[1,2-a]indazol-9 (1H)-one; 2-methyl-6-(6-(2-morpholinopyrimidin-5-yl)-9-oxo-1,2,3,9-tetrahydropyrazolo[1,2-a]indazol-3-yl)benzonitrile; 6-(2-morpholinopyrimidin-5-yl)-3-phenyl-2,3-dihydropyrazolo[1,2-a]indazol-9 (1H)-one; 4-methoxy-3-(6-(2-morpholinopyrimidin-5-yl)-9-oxo-1,2,3,9-tetrahydropyrazolo[1,2-a]indazol-3-yl)benzonitrile; 2-methoxy-3-(6-(2-morpholinopyrimidin-5-yl)-9-oxo-1,2,3,9-tetrahydropyrazolo[1,2-a]indazol-3-yl)benzonitrile; 3-(1-isopropyl-1H-pyrazol-5-yl)-6-(2-morpholinopyrimidin-5-yl)-2,3-dihydropyrazolo[1,2-a]indazol-9 (1H)-one; 2-methyl-6-(6-(2-morpholinopyrimidin-5-yl)-9-oxo-1,2,3,9-tetrahydropyrazolo[1,2-a]indazol-3-yl)benzamide; rac-(1R,9bR)-1-(2-(difluoromethoxy)phenyl)-8-(2-morpholinopyrimidin-5-yl)-2,3-dihydro-1H-pyrrolo[2, 1-a]isoindol-5 (9bH)-one; rac-(1R,9bS)-1-(2-(difluoromethoxy)phenyl)-8-(2-morpholinopyrimidin-5-yl)-2,3-dihydro-1H-pyrrolo[2,1-a]isoindol-5 (9bH)-one; rac-(1R, 10bR)-1-(2-(difluoromethoxy)phenyl)-9-(2-morpholinopyrimidin-5-yl)-3,4-dihydro-1H-[1,4]oxazino [3,4-a]isoindol-6 (10bH)-one; (1S,9bS)-1-(2-(difluoromethoxy)phenyl)-8-(2-morpholinopyrimidin-5-yl)-2,3-dihydro-1H-pyrrolo[2,1-a]isoindol-5 (9bH)-one; (1R,9bR)-1-(2-(difluoromethoxy)phenyl)-8-(2-morpholinopyrimidin-5-yl)-2,3-dihydro-1H-pyrrolo[2,1-a]isoindol-5 (9bH)-one; (1S,9bS)-1-(2-(difluoromethoxy)phenyl)-8-(2-(2-hydroxypropan-2-yl)pyrimidin-5-yl)-2,3-dihydro-1H-pyrrolo[2,1-a]isoindol-5 (9bH)-one; (1R,9bR)-1-(2-(difluoromethoxy)phenyl)-8-(2-(2-hydroxypropan-2-yl)pyrimidin-5-yl)-2,3-dihydro-1H-pyrrolo[2,1-a]isoindol-5 (9bH)-one; (1S,9bS)-1-(2-(difluoromethoxy)phenyl)-8-(2-((R)-2-(methoxymethyl)pyrrolidin-1-yl)pyrimidin-5-yl)-2,3-dihydro-1H-pyrrolo[2,1-a]isoindol-5 (9bH)-one; (1R,9bR)-1-(2-(difluoromethoxy)phenyl)-8-(2-((R)-2-(methoxymethyl)pyrrolidin-1-yl)pyrimidin-5-yl)-2,3-dihydro-1H-pyrrolo[2, 1-a]isoindol-5 (9bH)-one; (1R,9bR)-1-(2-(difluoromethoxy)phenyl)-8-(2-((R)-3-oxohexahydroimidazo[1,5-a]pyrazin-7 (1H)-yl)pyrimidin-5-yl)-2,3-dihydro-1H-pyrrolo[2,1-a]isoindol-5 (9bH)-one; (1R,9bR)-1-(2-(difluoromethoxy)phenyl)-8-(2-((S)-3-oxohexahydroimidazo[1,5-a]pyrazin-7 (1H)-yl)pyrimidin-5-yl)-2,3-dihydro-1H-pyrrolo[2, 1-a]isoindol-5 (9bH)-one; (IR)-1-(2-(difluoromethoxy)phenyl)-8-(2-(2-hydroxypropan-2-yl)-4-methylpyrimidin-5-yl)-2,3-dihydro-1H-pyrrolo[2,1-a]isoindol-5 (9bH)-one; (R)-6-(2-((R)-4-acetyl-2-methylpiperazin-1-yl)pyrimidin-5-yl)-3-(2-(difluoromethoxy)phenyl)-2,3-dihydropyrazolo[1,2-a]indazol-9 (1H)-one; (S)-3-(2-(difluoromethoxy)phenyl)-6-(2-((S)-3-oxohexahydroimidazo[1,5-a]pyrazin-7 (1H)-yl)pyrimidin-5-yl)-2,3-dihydropyrazolo[1,2-a]indazol-9 (1H)-one; (R)-3-(2-(difluoromethoxy)-5-methylphenyl)-6-(2-(2-hydroxypropan-2-yl)pyrimidin-5-yl)-2,3-dihydropyrazolo[1,2-a]indazol-9 (1H)-one; (R)-6-(2-((R)-4-acetyl-3-methylpiperazin-1-yl)pyrimidin-5-yl)-3-(2-(difluoromethoxy)phenyl)-2,3-dihydropyrazolo[1,2-a]indazol-9 (1H)-one; (R)-3-(2-(difluoromethoxy)-5-methylphenyl)-6-(2-((S)-3-oxohexahydroimidazo[1,5-a]pyrazin-7 (1H)-yl)pyrimidin-5-yl)-2,3-dihydropyrazolo[1,2-a]indazol-9 (1H)-one; (R)-3-(2-(difluoromethoxy)phenyl)-6-(2-((R)-4-(2-hydroxyacetyl)-3-methylpiperazin-1-yl)pyrimidin-5-yl)-2,3-dihydropyrazolo[1,2-a]indazol-9 (1H)-one; (R)-3-(2-(difluoromethoxy)-5-methylphenyl)-6-(2-(2-hydroxypropan-2-yl)pyrimidin-5-yl)-2,3-dihydropyrazolo[1,2-a]indazol-9 (1H)-one; (S)-3-(2-(difluoromethoxy)phenyl)-6-(2-((S)-3-oxohexahydroimidazo[1,5-a]pyrazin-7 (1H)-yl)pyrimidin-5-yl)-2,3-dihydropyrazolo[1,2-a]indazol-9 (1H)-one; (S)-3-(2-(difluoromethoxy)phenyl)-6-(2-(2-hydroxypropan-2-yl)pyrimidin-5-yl)-2,3-dihydropyrazolo[1,2-a]indazol-9 (1H)-one; (R)-3-(2-(difluoromethoxy)phenyl)-6-(2-((R)-3-hydroxy-4-(2-hydroxyacetyl)piperazin-1-yl)pyrimidin-5-yl)-2,3-dihydro-1H,9H-pyrazolo[1,2-a]indazol-9-one; (S)-3-(2-(difluoromethoxy)-5-methylphenyl)-7-fluoro-6-(2-(2-hydroxypropan-2-yl)pyrimidin-5-yl)-2,3-dihydropyrazolo[1,2-a]indazol-9 (1H)-one; (R)-3-(2-(difluoromethoxy)-5-methylphenyl)-7-fluoro-6-(2-(2-hydroxypropan-2-yl)pyrimidin-5-yl)-2,3-dihydropyrazolo[1,2-a]indazol-9 (1H)-one; (R)-3-(2-(difluoromethoxy)phenyl)-7-fluoro-6-(2-(2-hydroxypropan-2-yl)pyrimidin-5-yl)-2,3-dihydropyrazolo[1,2-a]indazol-9 (1H)-one; (S)-3-(2-(difluoromethoxy)phenyl)-7-fluoro-6-(2-(2-hydroxypropan-2-yl)pyrimidin-5-yl)-2,3-dihydropyrazolo[1,2-a]indazol-9 (1H)-one; 3-(5-(hydroxymethyl)-2-methoxyphenyl)-6-(2-morpholinopyrimidin-5-yl)-2,3-dihydropyrazolo[1,2-a]indazol-9 (1H)-one; (R)-3-(2-(difluoromethoxy)-5-methylphenyl)-7-fluoro-6-(2-(2-hydroxypropan-2-yl)pyrimidin-5-yl)-2,3-dihydro-1H,9H-pyrazolo[1,2-a]indazol-9-one; (S)-3-(2-(difluoromethoxy)-5-methylphenyl)-7-fluoro-6-(2-(2-hydroxypropan-2-yl)pyrimidin-5-yl)-2,3-dihydro-1H,9H-pyrazolo[1,2-a]indazol-9-one; (S)-3-(2-(difluoromethoxy)phenyl)-6-(2-(2-hydroxypropan-2-yl)pyrimidin-5-yl)-2,3-dihydro-1H,9H-pyrazolo[1,2-a]indazol-9-one; (R)-3-(2-(difluoromethoxy)-5-methylphenyl)-6-(2-((S)-3-oxohexahydroimidazo[1,5-a]pyrazin-7 (1H)-yl)pyrimidin-5-yl)-2,3-dihydro-1H,9H-pyrazolo[1,2-a]indazol-9-one; (R)-3-(2-(difluoromethoxy)-5-methylphenyl)-6-(2-(2-hydroxypropan-2-yl)pyrimidin-5-yl)-2,3-dihydro-1H,9H-pyrazolo[1,2-a]indazol-9-one; (R)-6-(2-((R)-4-acetyl-3-methylpiperazin-1-yl)pyrimidin-5-yl)-3-(2-(difluoromethoxy)phenyl)-2,3-dihydropyrazolo[1,2-a]indazol-9 (1H)-one; (R)-3-(2-(difluoromethoxy)phenyl)-6-(2-((R)-3-hydroxy-4-(2-hydroxyacetyl)piperazin-1-yl)pyrimidin-5-yl)-2,3-dihydropyrazolo[1,2-a]indazol-9 (1H)-one; 9b-(2-methoxyphenyl)-8-(2-morpholinopyrimidin-5-yl)-2,3-dihydro-1H-pyrrolo[2,1-a]isoindol-5 (9bH)-one; or 3-(5-(hydroxymethyl)-2-methoxyphenyl)-6-(2-morpholinopyrimidin-5-yl)-2,3-dihydropyrazolo[1,2-a]indazol-9 (1H)-one.

### Proprotein Convertase Subtilisin/Kexin Type 9 (PCSK-9)

In some embodiments, the Target Extracellular Protein is human proprotein convertase subtilisin/kexin type 9 (PCSK-9) (UniProtKB-Q8NBP7 (PCSK9_HUMAN)). PCSK-9 is a crucial player in the regulation of plasma cholesterol homeostasis. PCSK-9 binds to low-density lipid receptor family members: low density lipoprotein receptor (LDLR), very low-density lipoprotein receptor (VLDLR), apolipoprotein E receptor (LRP1/APOER) and apolipoprotein receptor 2 (LRP8/APOER2), and promotes their degradation in intracellular acidic compartments. It acts via a non-proteolytic mechanism to enhance the degradation of the hepatic LDLR through a clathrin LDLRAP1/ARH-mediated pathway, and may prevent the recycling of LDLR from endosomes to the cell surface or direct it to lysosomes for degradation. PCSK-9 has been implicated in high blood cholesterol and the development of cardiovascular disease.

The Protein Data Bank website provides the crystal structure of PCSK-9 searchable by 2P4E (Cunningham, D., et al., Nat Struct Mol Biol., 2007, 14 413-419); as well as the crystal structure of PCSK-9 bound to various compounds searchable by 3BPS (Kwon, H. J., et al., Proc Natl Acad Sci USA, 2008, 105 1820-1825); 6U26, 6U2N, 6U2P, 6U36, 6U38, and 6U3X (Petrilli, W. L., et al., Cell Chem Biol., 2019, 27 32-40.e3); 50CA (Gustafsen, C., et al., Nat Commun., 2017, 8 503-503); 4NE9 (Schroeder, C. I., et al., Chem Biol., 2014, 21 284-294); 40V6 (Mitchell, T., et al., J Pharmacol Exp Ther., 2014, 350 412-424); and 4NMX (Zhang, Y., et al., J Biol Chem., 2014, 289 942-955). Additionally, Piper et al., provides insight into the crystal structure of PCSK9 (Piper, D. E., et al., Structure, 2007, 15 (5), 545-52).

Representative PCSK-9 Targeting Ligands are provided in FIG. 1. In some embodiments, the PCSK-9 Targeting Ligand is the peptide TVFTSWEEYLDWV (SEQ ID:3) (J. Bio. Chem. 2014 January; 289 (2):942-955, incorporated herein by reference). Additional PCSK-9 Targeting Ligands are provided in, for example, U.S. Pat. No. 9,227,956, J Biol Chem 289:942-55 (2014), each of which is incorporated by reference herein.

In certain embodiments the PCSK-9 ligand is any PCSK-9 ligand described in WO2021/156792 which is incorporated by reference.

Non-limiting examples of PCSK-9 Targeting Ligands that can be used in any of the formulas of the present invention include:

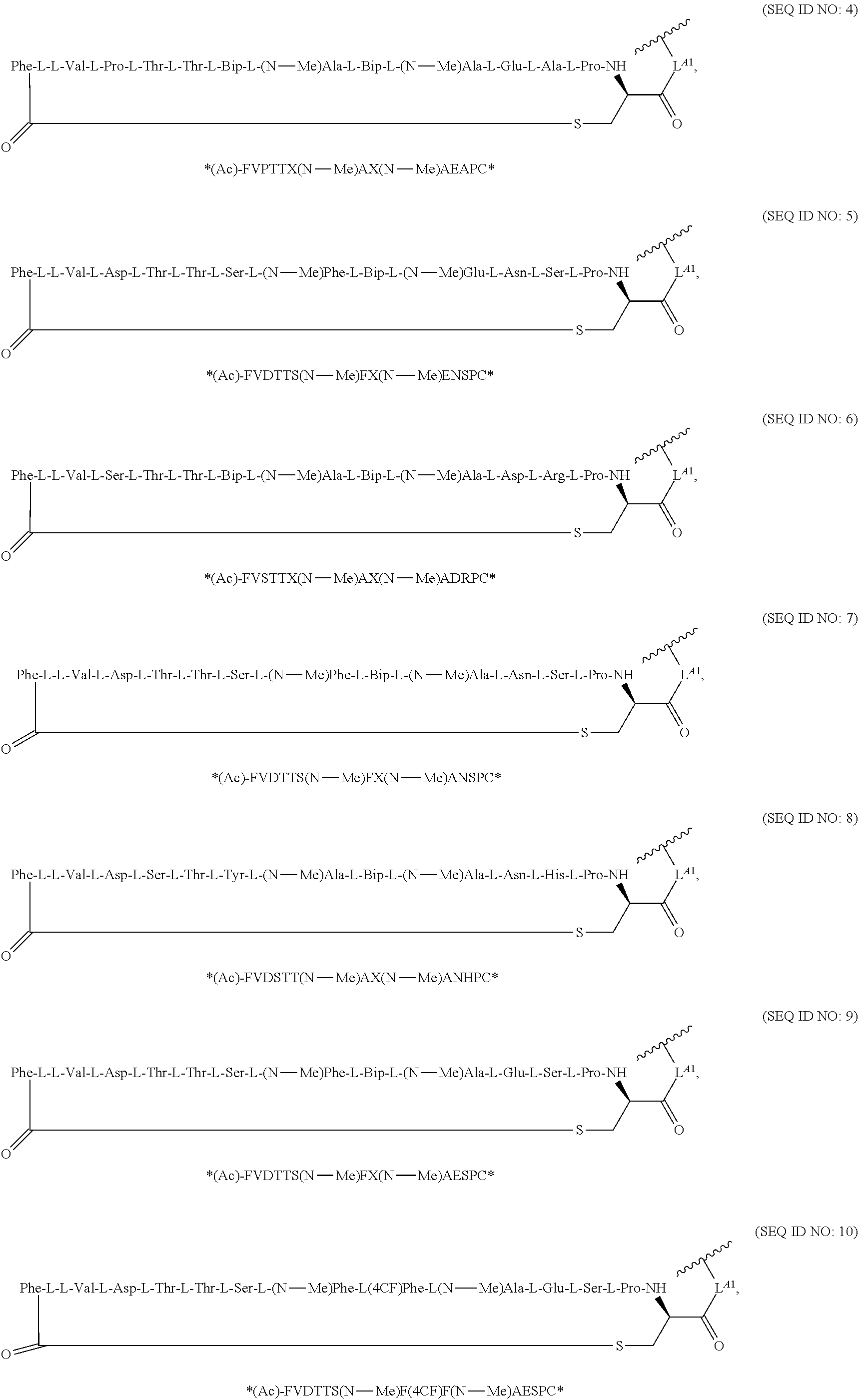

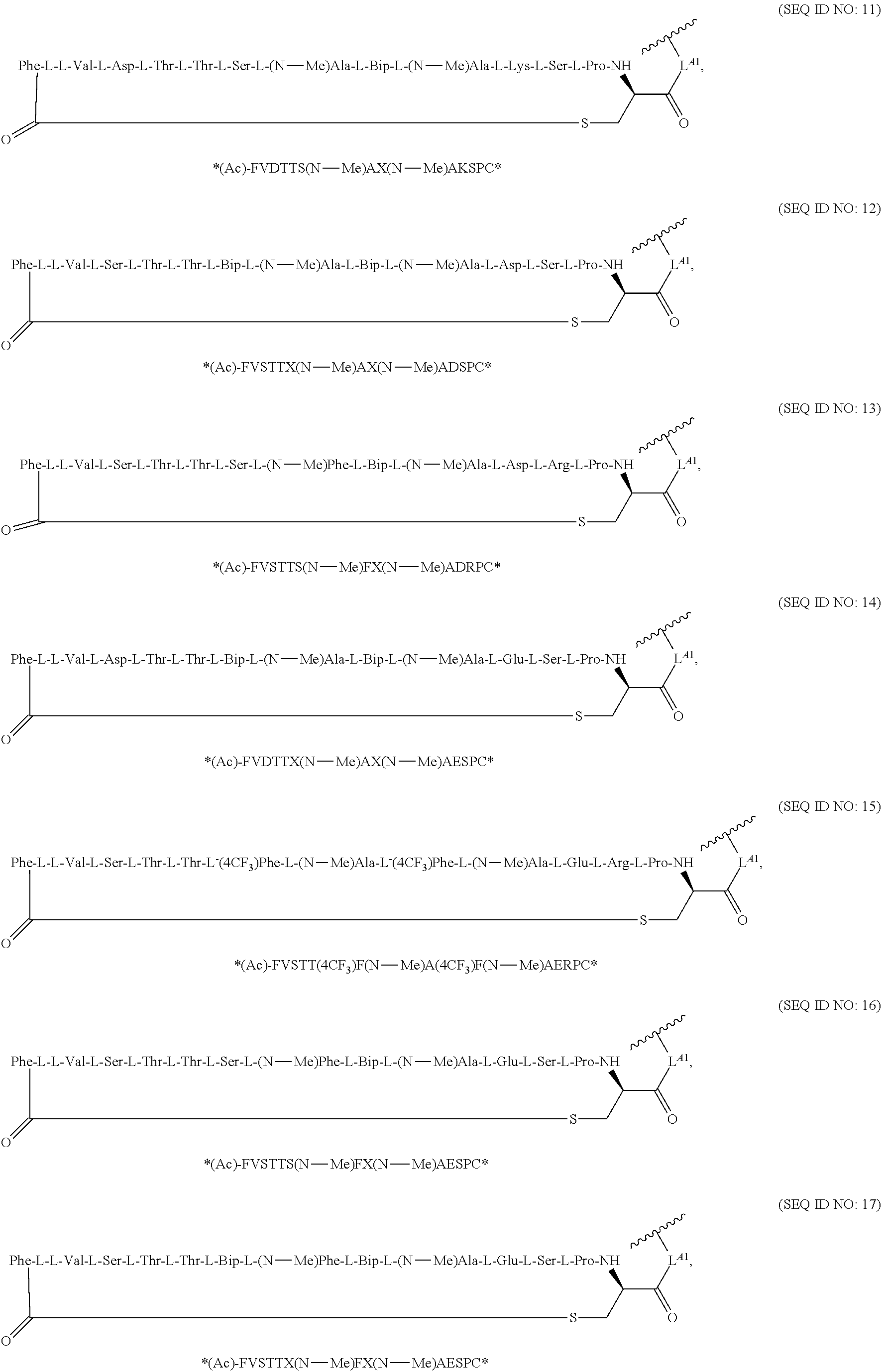

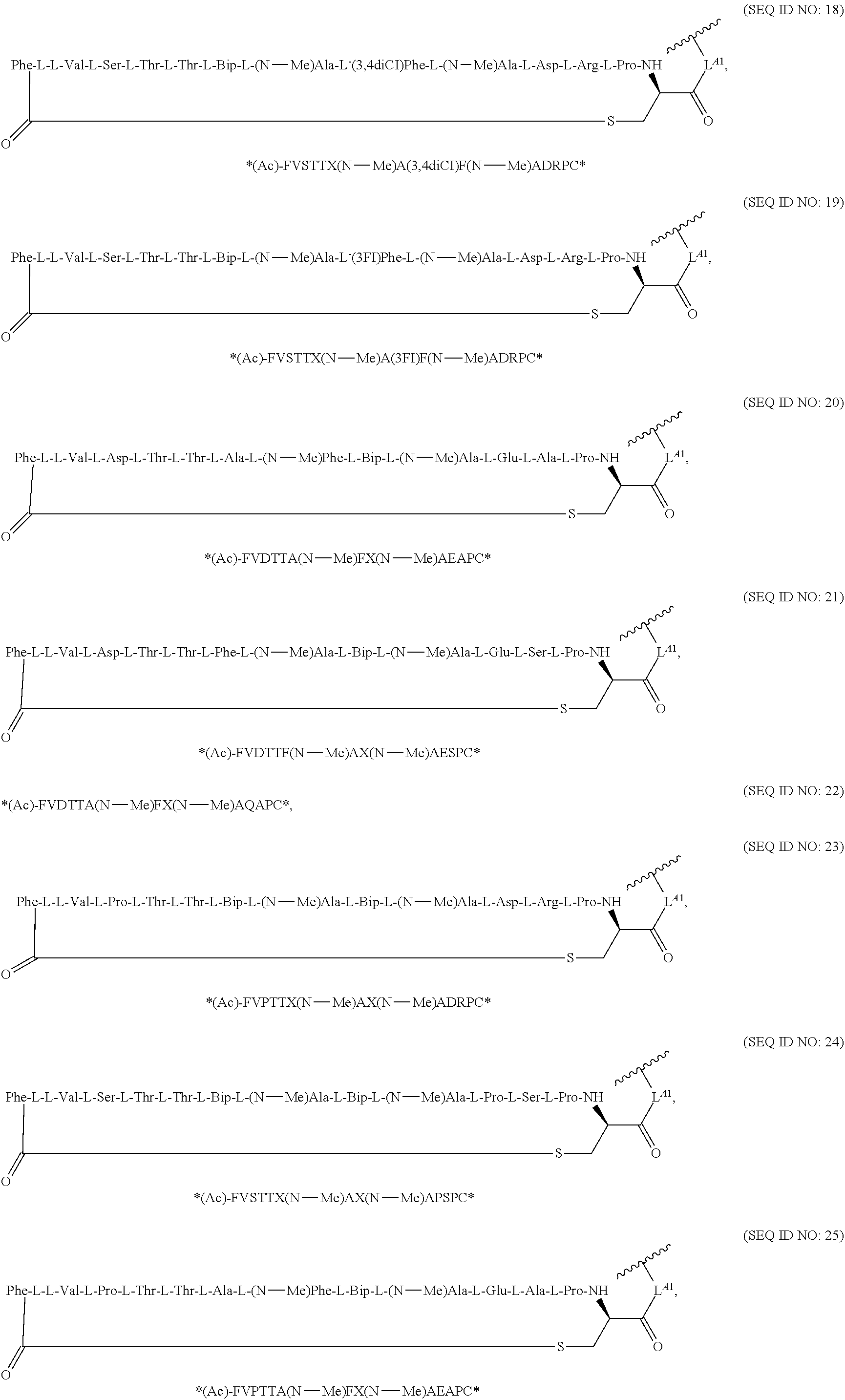

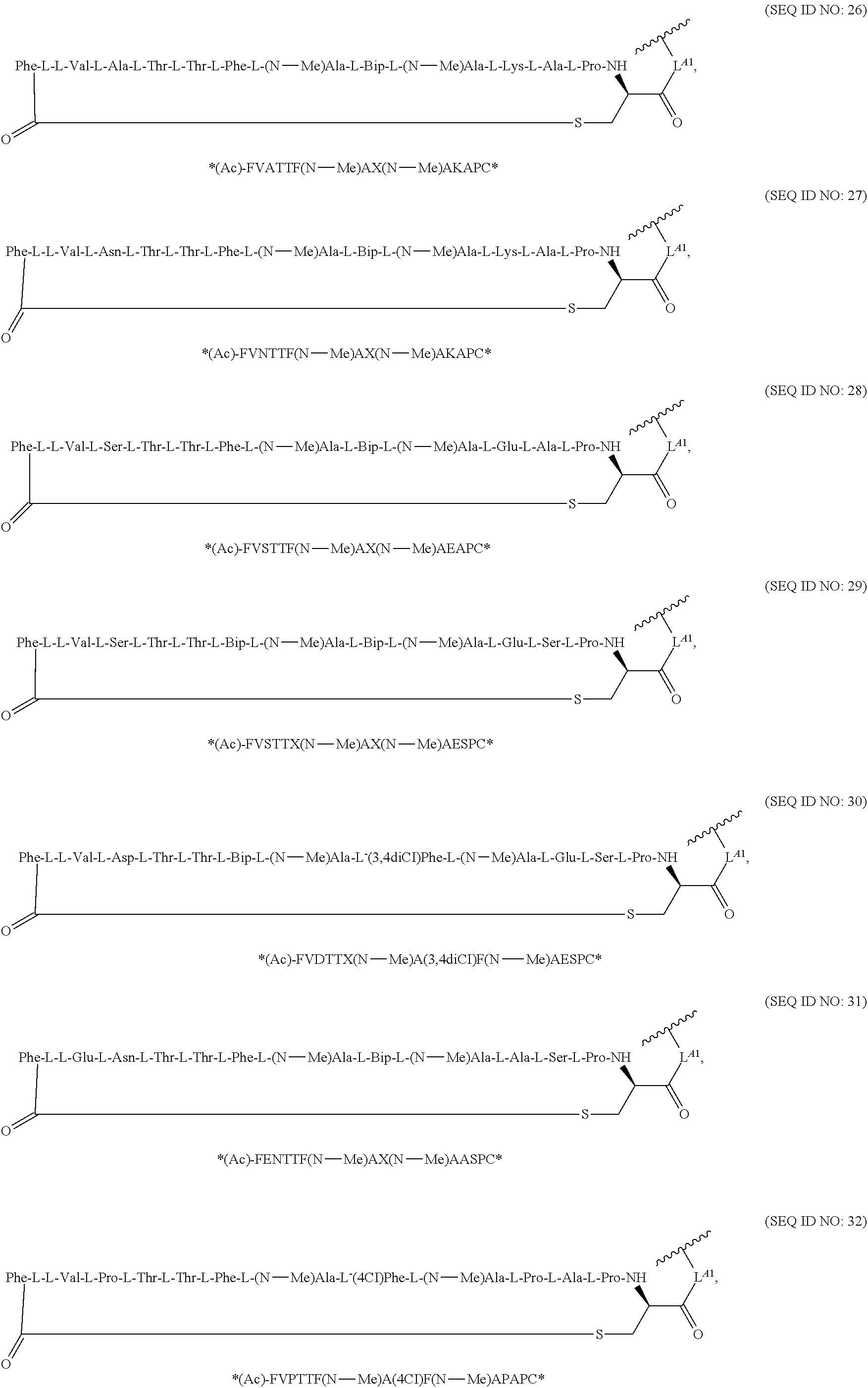

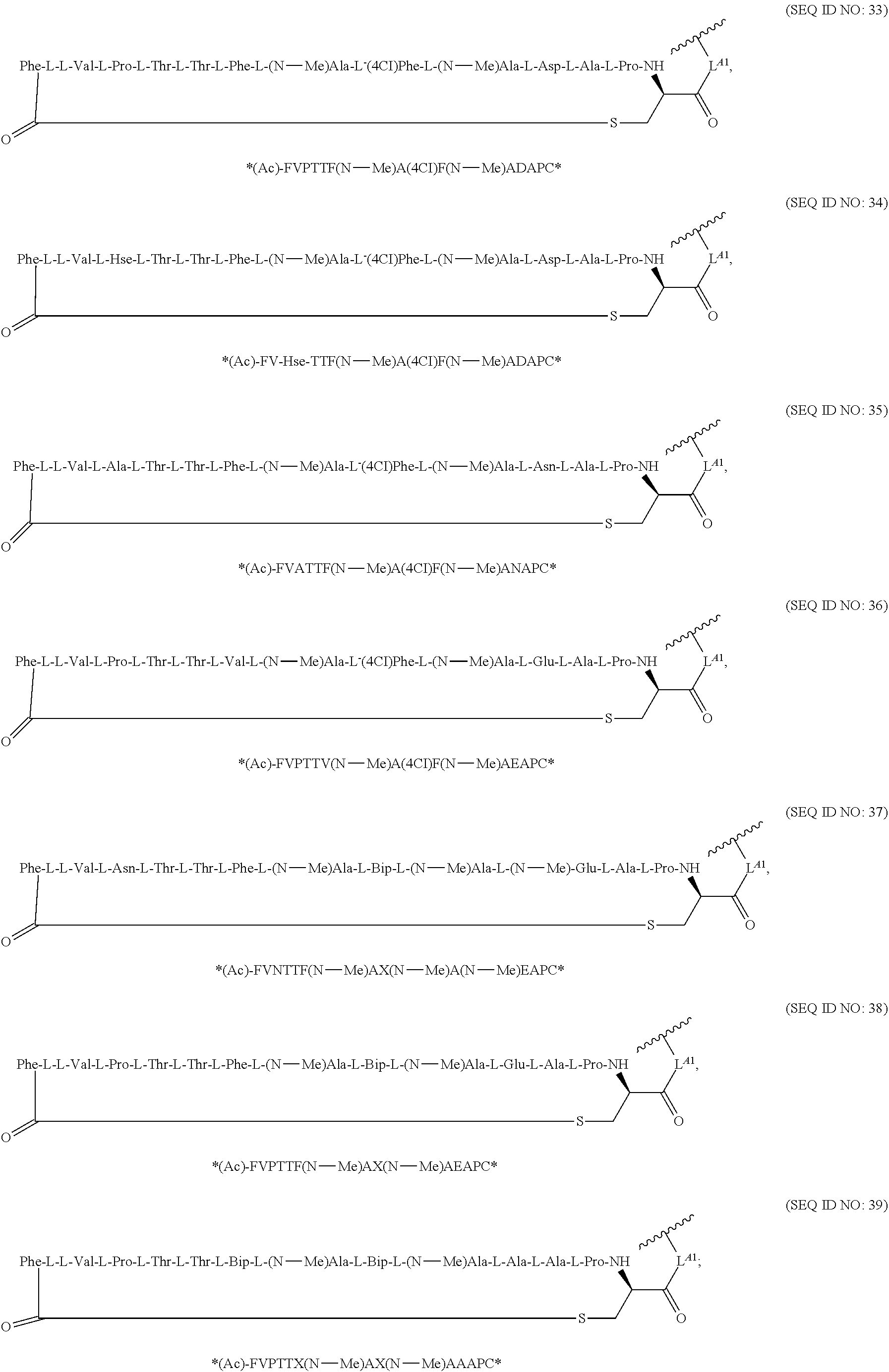

wherein L^{A1} is bond, NR⁸, or O; X, Bip, L-(4-phenyl-phenylalanine); N-Me, N-Methyl; *, cyclic bond.

In certain embodiments the PCSK9 Targeting Ligand is a compound of Formula:

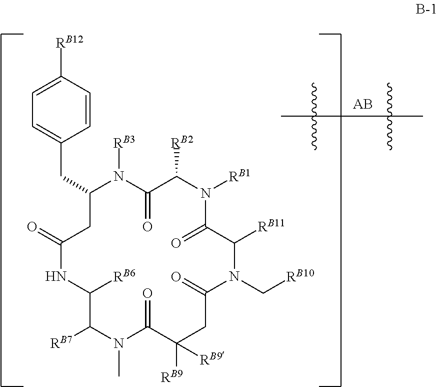

wherein;
R^{B1} is H;
R^{B2} is (C₁-C₆)alkoxy, —L^{B1}-, or (C₁-C₆)alkyl, substituted with —C(═O)OH;
R^{B3} is H or (C₁-C₆)alkyl;
R^{B6} is H, (C₁-C₆)alkyl or L^{B1};
R^{B7} is H, (C₁-C₆)alkyl or L^{B1};
or R^{B6} and R^{B7} together with the carbon atoms to which they are attached form a (C₃-C₇)cycloalkyl;
R^{B9} is H or (C₁-C₆)alkyl, optionally substituted with one or more R^{B27};
R^{B9}′ is H or (C₁-C₆)alkyl;
R^{B10} is (C₆-C₁₀) aryl substituted with OR^{B13} and optionally substituted with one or more R^{B14};
R^{E1} is (C₁-C₆)alkyl or L^{B1};
R^{B12} is halogen, (C₁-C₆)alkyl, (C₁-C₆)alkoxy, (C₁-C₆)haloalkyl, (C₁-C₆)haloalkoxy, —OH, or CN;
R^{B13} is (C₆-C₁₀) aryl substituted with R^{B16}.
each R^{B14} is independently at each occurrence halogen, (C₁-C₆)alkyl, (C₁-C₆)alkoxy; (C₁-C₆)haloalkyl, (C₁-C₆)haloalkoxy, oxo, —OH, or CN;
R^{B16} is 5- to 7-membered heteroaryl comprising 1-3 heteroatoms selected from N, 0, and S, optionally substituted with one or more R^{B26}; each R^{B26} is independently at each occurrence (C₁-C₆)alkyl optionally substituted with one or more R^{B29};
each R^{B27} is independently at each occurrence (C₆-C₁₀) aryl;
each R^{B29} is independently at each occurrence —NR^{B31}R^{B32} or 4- to 7-membered heterocyclyl comprising 1-3 heteroatoms selected from N, O, and S;
each R^{B31} is independently selected from Hand (C₁-C₆)alkyl;
each R^{B32} is independently selected from Hand (C₁-C₆)alkyl;
L^{B1} is —(CH₂)ₚNH—*, where the* of L^{B1} indicates the point of attachment to Linker (LA), and where at least one of R^{B11}, R^{B6} or R^{B7} is —L^{B1} -; and
n is 1.

In certain embodiments the PCSK9 Targeting Ligand is a compound of Formula:

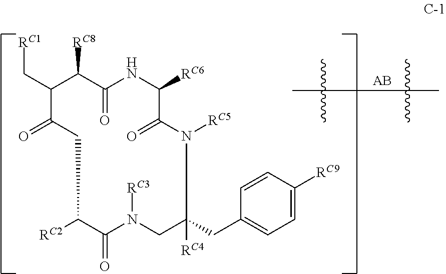

wherein,
R^{C1} is (C₆-C₁₀) aryl substituted with —OR^{C10} and one or more R^{C11};
R^{C2} is H, (C₁-C₆)alkyl, —L^{C1} or (C₃-C₉) carbocyclyl, wherein the alkyl is substituted with one R^{C18}, and the carbocyclyl is substituted with one or more R^{C19};
R^{C3} is H or (C₁-C₆)alkyl;
R^{C4} is H or (C₁-C₆)alkyl; or R^{C3} and R^{C4} together with the atoms to which they are attached form a 5—to 7-membered heterocyclyl ring comprising 1-3 heteroatoms selected from N, O, and S;
R^{C5} is H or (C₁-C₆)alkyl;
R^{C6} is (C₁-C₆)alkyl, or —L^{C1}, wherein the alkyl is optionally substituted with one or more substituents each independently selected from —OH or (C₁-C₆)alkoxy;
R^{C8} is H, (C₁-C₆)alkyl, or —L^{C1};
R^{C9} is halogen;
R^{C10} is (C₆-C₁₀) aryl substituted with one R^{C22}.
each R^{C11} is independently at each occurrence halogen, (C₁-C₆)alkyl, (C₁-C₆)alkoxy, (C₁-C₆)haloalkyl, (C₁-C₆)haloalkoxy, —OH, or CN;
R^{C18} is (C₆-C₁₀) aryl;
each R^{C19} is independently at each occurrence halogen, (C₁-C₆)alkyl, (C₁-C₆)alkoxy; (C₁-C₆)haloalkyl, (C₁-C₆)haloalkoxy, —OH, or CN;
R^{C22} is 5- or 6-membered heteroaryl comprising 1-3 heteroatoms selected from N, 0, and S, substituted with one or more R^{C23};
each R^{C23} is independently at each occurrence (C₁-C₆)alkyl, optionally substituted with —NR^{C24}R^{C25} or a 4- to 7-membered heterocyclyl comprising 1-3 heteroatoms selected from N, 0, and S;
R^{C24} is H, (C₁-C₆)alkyl;
R^{C25} is H, (C₁-C₆)alkyl,
L^{C1} is —(CH₂)ₚNH—*, where the* of L^{C1} indicates the point of attachment to Linker (LA), and where at least one of R^{C2}, R^{C6} or R^{C8} is —L^{C1}; and
p is 1, 2, 3, 4,5, or 6.

In certain embodiments the PCSK-9 Targeting Ligands that can be used in any of the formulas of the present invention include:

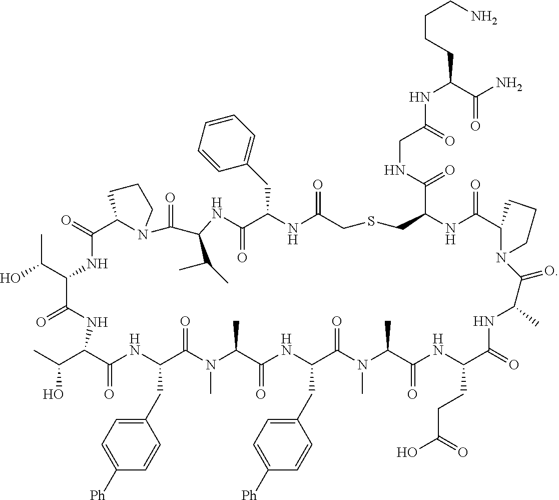 [SMILES: CC(C)[C@@H]1NC(=O)[C@H](Cc2ccccc2)NC(=O)CSC[C@@H](C(=O)NCC(=O)N[C@@H](CCCCN)C(N)=O)NC(=O)[C@@H]2CCCN2C(=O)[C@H](C)NC(=O)[C@H](CCC(=O)O)NC(=O)[C@H](C)N(C)C(=O)[C@H](Cc2ccc(-c3ccccc3)cc2)NC(=O)[C@H](C)N(C)C(=O)[C@H](Cc2ccc(-c3ccccc3)cc2)NC(=O)[C@H]([C@@H](C)O)NC(=O)[C@H]([C@@H](C)O)NC(=O)[C@@H]2CCCN2C1=O]

In certain embodiments the PCSK-9 Targeting Ligands that can be used in any of the formulas of the present invention include:

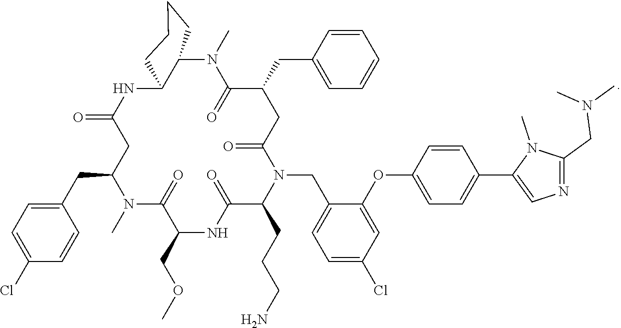

In certain embodiments the compound of the present invention is selected from

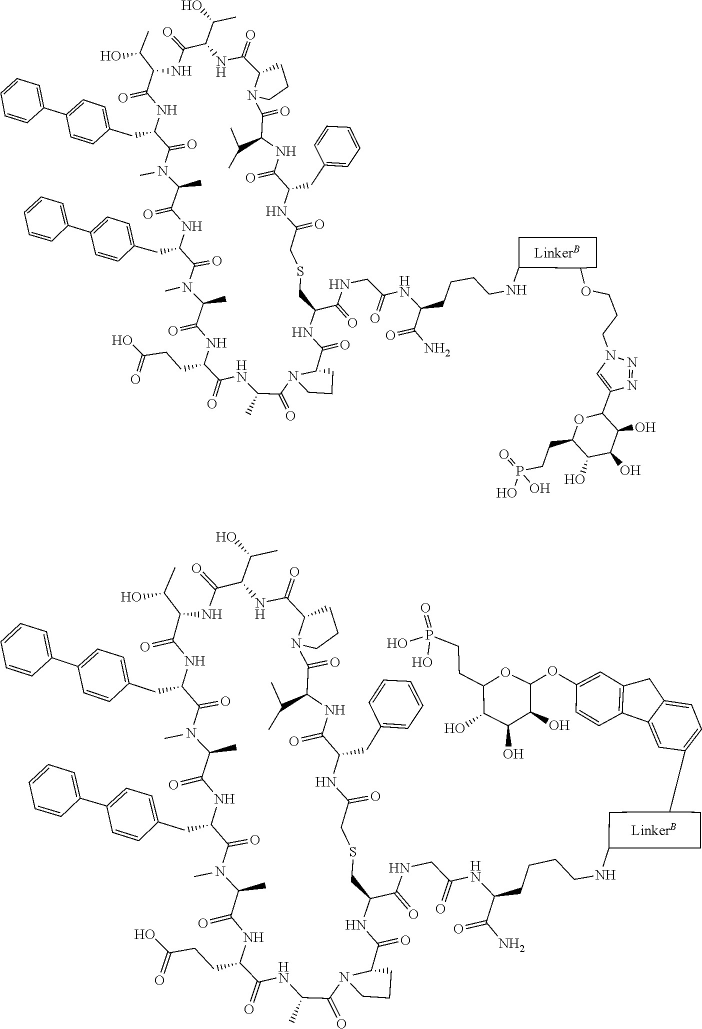

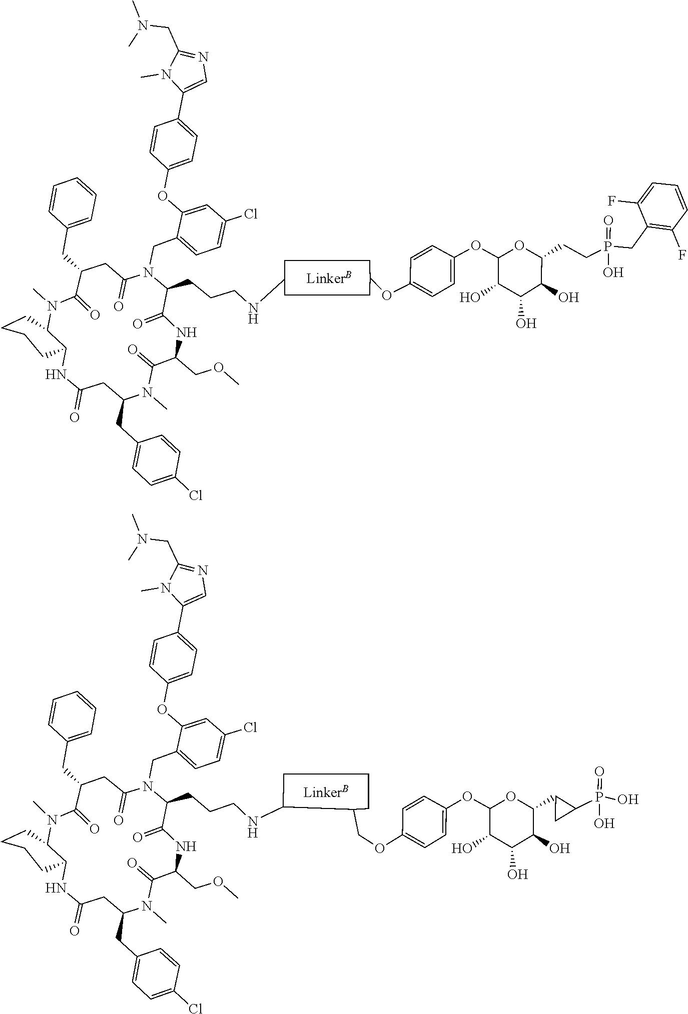

In certain embodiments the compound of the present invention is selected from

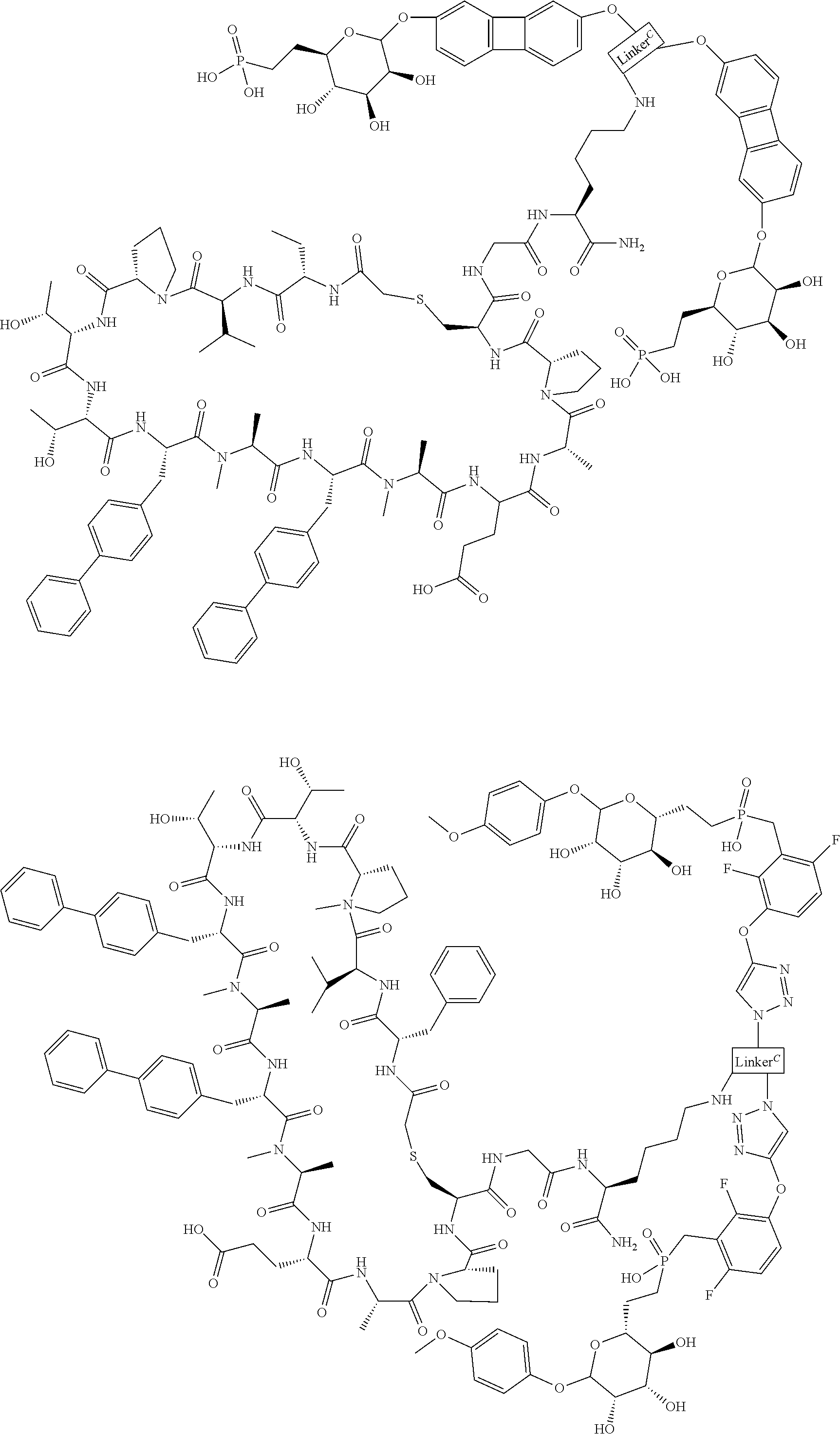

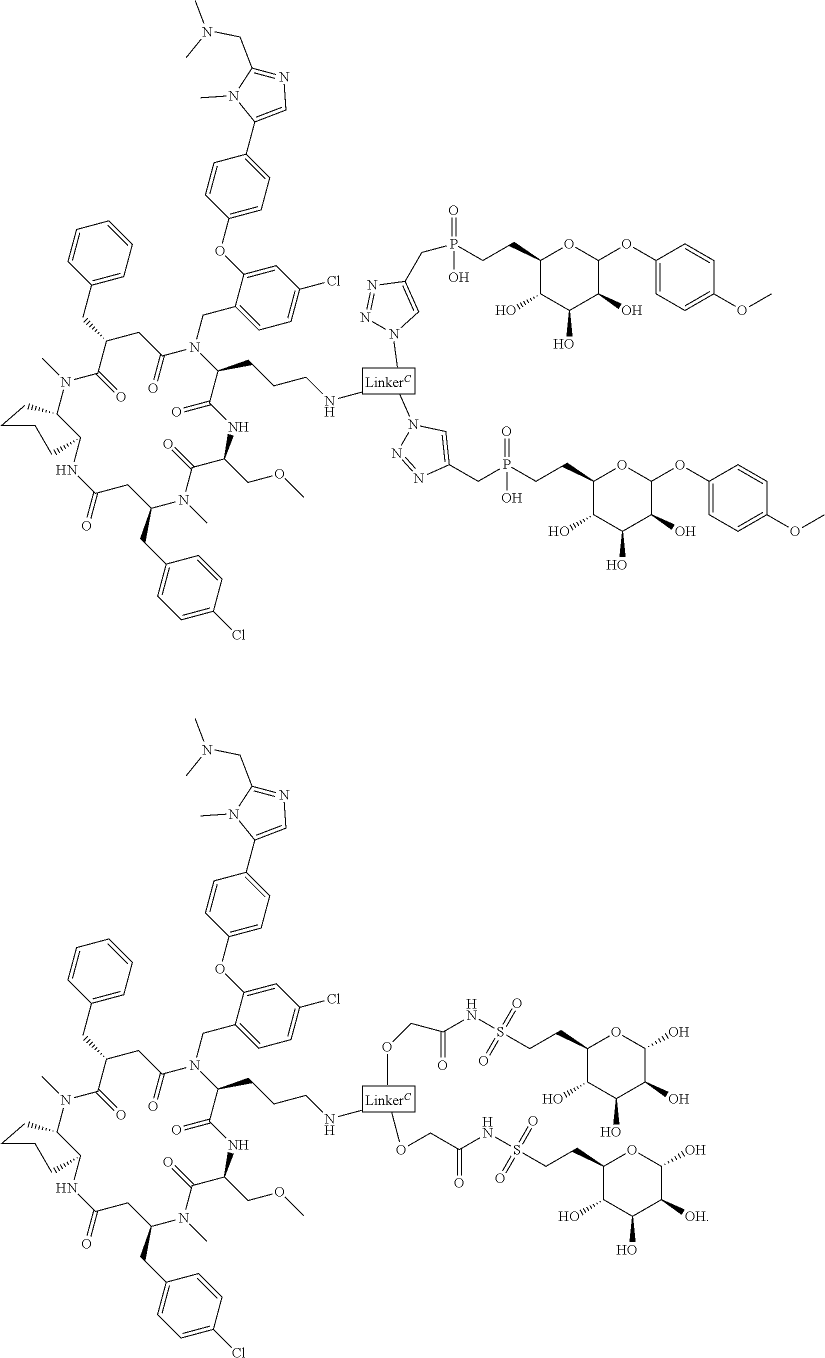

In certain embodiments the PCSK9 Targeting Ligand is selected from:

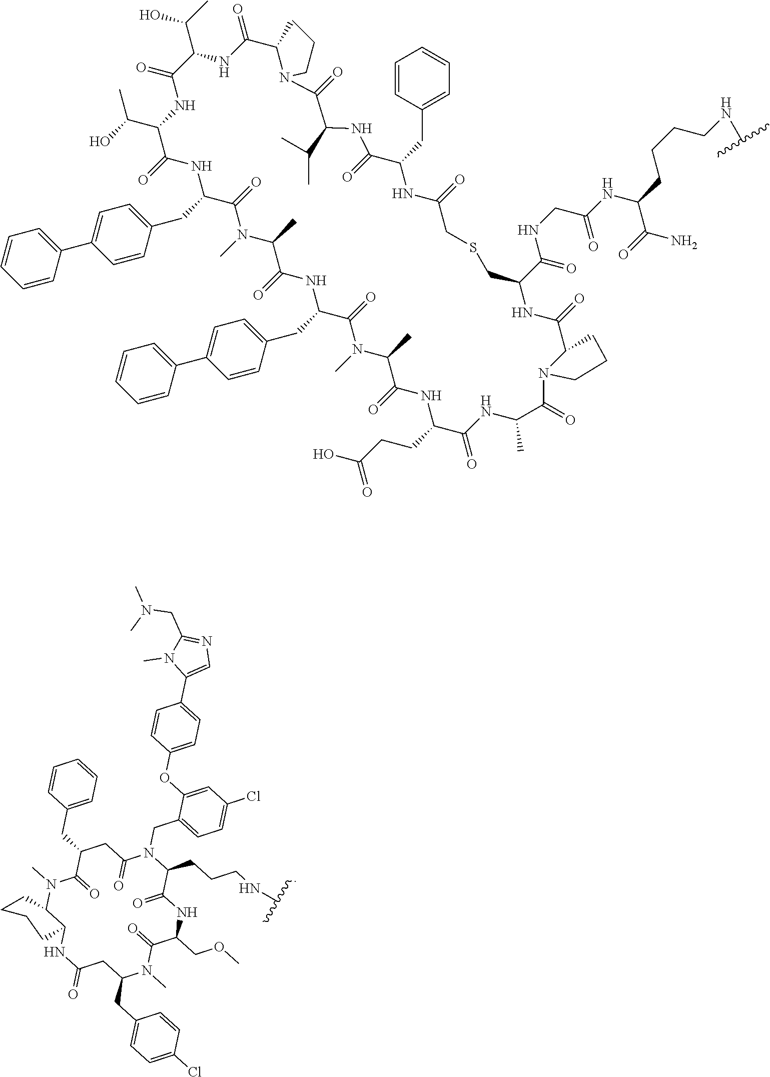

### Neonatal Fragment Crystallizable Receptor (FcRN)

In certain embodiments, the Extracellular Protein Targeting Ligand binds the neonatal fragment crystallizable receptor (FcRN). In certain embodiments, the FcRN Targeting Ligand is selected from a ligand described in Anderrsen, J. “Seclection of Nanbodies that Target Human Neonatal Fc Receptor”, Scientific Reports, 2013, 3:1118; WO 2022046614; U.S. Pat. No. 8,101,186B2; WO 2021074683; WO2020245420A1; Gevys, A. “A human endothelial cell-based recycling assay for screening of FcRn targeted molecules” Nature Communications 2018, 9:621; McDonnell, K. “Synthesis and Structure-Activity Relationships of Dimeric Peptide Antagonists of the Human Immunoglobulin G-Human Neonatal Fc Receptor (IgG-FcRn) Interaction” J. Med. Chem. 2010, 53, 1587-1596; Mezo, A. “PEGylation enhances the therapeutic potential of peptide antagonists of the neonatal Fc receptor, FcRn” B.M.C.L. 21 (2011) 6332-6335; Mezo, A. “X-ray Crystal Structures of Monomeric and Dimeric Peptide Inhibitors in Complex with the Human Neonatal Fc Receptor, FcRn” J. Biol. Chem. 2010, 285:36, 27694-27701; Mezo, A. “Structure-activity relationships of a peptide inhibitor of the human FcRn: human IgG interaction” Bioorganic Med. Chem. 16 (2008) 6394-6405; Mezo, A. “Reduction of IgG in nonhuman primates by a peptide antagonist of the neonatal Fc receptor FcRn” PNAS 2008, 105:7, 2337-2342; Zhu, L. “FcRn inhibitors: a novel option for the treatment of myasthenia gravis” Neural Regeneration Research 2023, 18:8, 1637-1644; Wang, Z. “Discovery and structure-activity relationships of small molecules that block the human immunoglobulin G-human neonatal Fc receptor (hIgG-hFcRn) protein-protein interaction” B.M.C.L. 23, 2013, 1253-1256; Stoppler, D. “Insight into small molecule binding to the neonatal Fc receptor by X-ray crystallography and 100 kHz magic-angle-spinning NMR” PLoS Biology 16 (5), e2006192; Seijsing, J. et al. “In vivo depletion of serum IgG by an affibody molecule binding the neonatal Fc receptor” Scientific Reports 2018, 8:5141; and Nath, N. et al. “Deciphering the Interaction between Neonatal Fc Receptor and Antibodies Using a Homogeneous Bioluminescent Immunoassay” Journal of Immunology, 2021 207:4, 1211-1221.

Additional anti-B1AR autoantibody Targeting Ligands

In certain embodiments, the anti-β1AR autoantibody Targeting Ligand is SEQ ID NO: 420, cyclized from N-terminus to C-terminus and with a disulfide bond between the cysteine residues. In certain embodiments, the anti-β1AR autoantibody Targeting Ligand is SEQ ID NO: 419

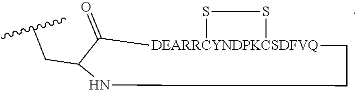

In certain embodiments, the anti-β1AR autoantibody Targeting Ligand has about 99%, 98%, 95%, 93%, 90%, 88%, 85%, 83%, or 80% sequence homology with SEQ ID NO: 419. In certain embodiments, the anti-BIAR autoantibody Targeting Ligand is SEQ ID NO: 419 modified with one, two, three, or four D-amino acids. In certain embodiments, the anti-β1AR autoantibody Targeting Ligand is

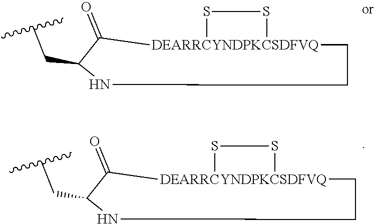

In certain embodiments the anti-β1AR autoantibody Targeting Ligand is SEQ ID NO: 400 5′-GGTTGGTGTGGTTGG-3′. In certain embodiments, the anti-β1AR autoantibody targeting ligand has about 93%, 86%, or 80% sequence homology with SEQ ID NO: 400. In other embodiments an aptamer described in Werner et al. “The aptamer BC 007 for treatment of dilated cardiomyopathy: evaluation in Doberman Pinschers of efficacy and outcomes” ESC Heart Failure, 2020, 7, 844-855 is used as an anti-B1AR autoantibody Targeting Ligand. In certain aspects an aptamer such as SEQ ID NO: 400 may provide lower immunogenicity than a protein-based targeting ligand.

In certain embodiments the anti-β1AR autoantibody Targeting Ligand is SEQ ID NO: 421 DCCKPDNYCR, wherein each amino acid is a D-amino acid. In certain embodiments, the anti-B1AR autoantibody targeting ligand has about 99%, 98%, 95%, 93%, 90%, 88%, 85%, 83%, or 80% sequence homology with SEQ ID NO: 421.

### Additional VEGFR-1 Targeting Ligands

In certain embodiments, the soluble VEGFR-1 Targeting Ligand is 01A04. The preparation of 01A04 is described in WO2016164567A1.

In certain embodiments, the VEGFR-1 Targeting Ligand is an antibody described in WO 2016/164528. In certain embodiments, the soluble VEGFR-1 Targeting Ligand is an antibody comprising one or more complementarity determining regions (CDR) selected from the group consisting of
a variable light (VL) chain CDR1 defined by an amino acid sequence having at least 80% identity to any one of SEQ ID NO: 440 to SEQ ID NO: 442,
a VL CDR2 defined by an amino acid sequence having at least 80% identity to any one of SEQ ID NO: 443 to SEQ ID NO: 445;
a VL CDR3 defined by an amino acid sequence having at least 80% identity to any one of SEQ ID NO: 446 to SEQ ID NO: 455;
a variable heavy (VH) chain CDR^{E} defined by an amino acid sequence having at least 80% identity to any one of SEQ ID NO: 422 to SEQ ID NO: 425,
a VH CDR2 defined by an amino acid sequence having at least 80% identity to any one of SEQ ID NO: 426 to SEQ ID NO: 435, and
a VH CDR3 defined by an amino acid sequence having at least 80% identity to any one of SEQ ID NO: 436 to SEQ ID NO: 439.

SEQ ID NO: 422
Ser Tyr Ala Met Ser
SEQ ID NO: 423
Asp Tyr Ser Met Ser
SEQ ID NO: 424
Asp Tyr Ser Ala Ser
SEQ ID NO: 425
Asp Tyr Ser Leu Ser
SEQ ID NO: 426
Ala Ile Ser Gly Ser Gly Gly Ser Thr Tyr Tyr Ala Asp Ser Val Lys
SEQ ID NO: 427
Ala Ile Ser Trp Asn Gly Asp Ser Thr Tyr Tyr Ala Glu Ser Met Lys
SEQ ID NO: 428
Ala Ile Ser Trp Asn Gly Asp Ser Thr Tyr Tyr Ala Glu Ser Leu Lys
SEQ ID NO: 429
Ala Ile Ser Trp Asn Gly Asp Ser Thr Tyr Tyr Ala Glu Ser Ala Lys
SEQ ID NO: 430
Ala Ile Ser Trp Asn Gly Asp Ser Thr Tyr Tyr Ala Glu Ser Val Lys
SEQ ID NO: 431
Ala Ile Thr Trp Ser Gly Asp Ser Thr Tyr Tyr Ala Glu Ser Val Lys
SEQ ID NO: 432
Ala Ile Ser Trp Ser Gly Asp Ser Thr Tyr Tyr Ala Glu Ser Leu Lys
SEQ ID NO: 433
Ala Ile Ser Trp Ser Gly Asp Ser Thr Tyr Tyr Ala Glu Ser Val Lys
SEQ ID NO: 434
Ala Ile Ser Trp Gln Gly Asp Ser Thr Tyr Tyr Ala Glu Ser Ala Lys
SEQ ID NO: 435
Ala Ile Ser Trp Asn Ala Asp Ser Thr Tyr Tyr Ala Glu Ser Ala Lys
SEQ ID NO: 436
Asp Tyr
SEQ ID NO: 437
Ser Trp Ala Thr Pro Ile Glu Ser Leu Tyr Tyr Tyr Gly Met Asp Tyr
SEQ ID NO: 438
Ser Trp Ala Thr Pro Ile Glu Ser Leu Tyr Tyr Tyr Gly Ser Asp Tyr
SEQ ID NO: 439
Ser Trp Ala Thr Pro Ile Glu Ser Leu Tyr Tyr Tyr Gly Thr Asp Tyr
SEQ ID NO: 440
Gly Gly Asn Asn Ile Gly Ser Lys Asn Val His
SEQ ID NO: 441
Gly Gly Asn Asn Leu Gly Tyr Lys Ser Val His
SEQ ID NO: 442
Gly Gly Asn Asn Ile Gly Ser Gln Thr Ala Gln
SEQ ID NO: 443
Arg Asp Ser Asn Arg Pro Ser
SEQ ID NO: 444
Arg Asp Asn Asn Arg Pro Ser
SEQ ID NO: 445
Ala Asn Asn Arg Arg Pro Ser
SEQ ID NO: 446
Gln Val Val Val
SEQ ID NO: 447
Gln Val Trp Asp Gly Ser Thr Gln Ala Ile Val
SEQ ID NO: 448
Gln Val Trp Glu Asp Ser Thr Gln Ala Ile Val
SEQ ID NO: 449
Gln Val Trp Asp Glu Ser Thr Gln Ala Ile Val
SEQ ID NO: 450
Gln Val Trp Ala Ala Ser Thr Gln Ala Ile Val
SEQ ID NO: 451
Gln Val Trp Asp Asp Ser Thr Gln Ala Ile Val
SEQ ID NO: 452
Gln Val Trp Glu Ala Ser Thr Gln Ala Ile Val
SEQ ID NO: 453
Gln Val Trp Asp Ala Ser Thr Gln Ala Ile Val
SEQ ID NO: 454
Gln Val Trp Glu Glu Ser Thr Gln Ala Ile Val
SEQ ID NO: 455
Gln Val Trp Glu Gly Ser Thr Gln Ala Ile Val

In certain embodiments, the VEGFR-1 Targeting Ligand is an antibody described in US 2016/0347843 A1.

In some embodiments, CDRH1 comprises a sequence as set forth in SEQ ID NO: 456. In some embodiments, CDRH2 comprises a sequence as set forth in SEQ ID NO: 457. In some embodiments, CDRH3 comprises a sequence as set forth in SEQ ID NO: 458. CDRL1 comprises a sequence as set forth in SEQ ID NO: 459. In some embodiments, CDRL2 comprises a sequence as set forth in SEQ ID NO: 460. In some embodiments, CDRL3 comprises a sequence as set forth in SEQ ID NO: 461. For example, in some embodiments, CDRH1 comprises a sequence as set forth in SEQ ID NO: 456, CDRH2 comprises a sequence as set forth in SEQID NO: 457, CDRH3 comprises a sequence as set forth in SEQ ID NO: 458. CDRL1 comprises a sequence as set forth in SEQ ID NO: 459, CDRL2 comprises a sequence as set forth in SEQ ID NO: 460, and CDRL3 comprises a sequence as set forth in SEQ ID NO: 461.

In some embodiments, CDRH1 comprises a sequence as set forth in SEQ ID NO: 462. In some embodiments, CDRH2 comprises a sequence as set forth in SEQ ID NO: 463. In some embodiments, CDRH3 comprises a sequence as set forth in SEQ ID NO: 464. CDRL1 comprises a sequence as set forth in SEQ ID NO: 465. In some embodiments, CDRL2 comprises a sequence as set forth in SEQ ID NO: 466. In some embodiments, CDRL3 comprises a sequence as set forth in SEQ ID NO: 467. For example, in some embodiments, CDRH1 comprises a sequence as set forth in SEQ ID NO: 462, CDRH2 comprises a sequence as set forth in SEQ ID NO: 463, CDRH3 comprises a sequence as set forth in SEQ ID NO: 464, CDRL1 comprises a sequence as set forth in SEQ ID NO: 465, CDRL2 comprises a sequence as set forth in SEQ ID NO: 466, and CDRL3 comprises a sequence as set forth in SEQ ID NO: 467.

SEQ ID NO: 456
Asp Asp Tyr Met Asn
SEQ ID NO: 457
Trp Ile Asp Pro Glu Asn Gly Asp Thr Glu Tyr Ala Ser Lys Phe Gln
SEQ ID NO: 458
Gly Tyr Asp Tyr Phe Pro Phe Val Tyr
SEQ ID NO: 459
Arg Ser Ser Gln Asn Ile Val His Ser Asn Gly Asn Thr Tyr Leu Glu
SEQ ID NO: 460
Lys Val Ser Asn Arg Phe Ser
SEQ ID NO: 461
Phe Glin Gly Ser His Val Pro Phe Thr
SEQ ID NO: 462
Thr Ser Gly Met Ser
SEQ ID NO: 463
Trp Ile Asn Thr Tyr Ser Gly Glu Pro Thr Tyr Ala Asp Asp Phe Lys
SEQ ID NO: 464
Ser Arg Asn Asn Tyr Glu Gly Phe Ala Tyr
SEQ ID NO: 465
Lys Ser Ser Gln Ser Leu Leu Tyr Ser Ser Asn Gln Lys Asn Tyr Leu
SEQ ID NO: 466
Trp Ala Ser Thr Arg Glu Ser
SEQ ID NO: 467
Gln Gln Tyr Tyr Leu Tyr Pro Leu Thr

### Anti-citrullinated Protein Antibodies (ACPA)

In certain embodiments, the Extracellular Target Protein is anti-citrullinated protein antibodies (ACPA). In certain embodiments, a Targeted Protein Degrader that degrades ACPA can be used in the treatment of autoimmune diseases such as rheumatoid arthritis. In certain embodiments, the ACPA Targeting Ligand is selected from SEQ ID NO: 468-470 (Khatri, S. et al “Cyclic Citrullinated Peptide Aptamer Treatment Attenuates Collagen-Induced Arthritis” Biomacromolecules 2022, 23, 2126-2137). In certain embodiments, the ACPA Targeting Ligand is a cyclized form of SEQ ID NO: 468-470. In certain embodiments, the ACPS Targeting Ligand is a linear or cyclic peptide with at least about 99%, 98%, 95%, 92%, 90%, 85%, or 80% sequence homology with a peptide selected from SEQ ID NO: 468-470. In certain embodiments, the ACPS Targeting Ligand is a linear or cyclic peptide of SEQ ID NO 468-470 with one, two, three, or four amino acids modified with citrulline.

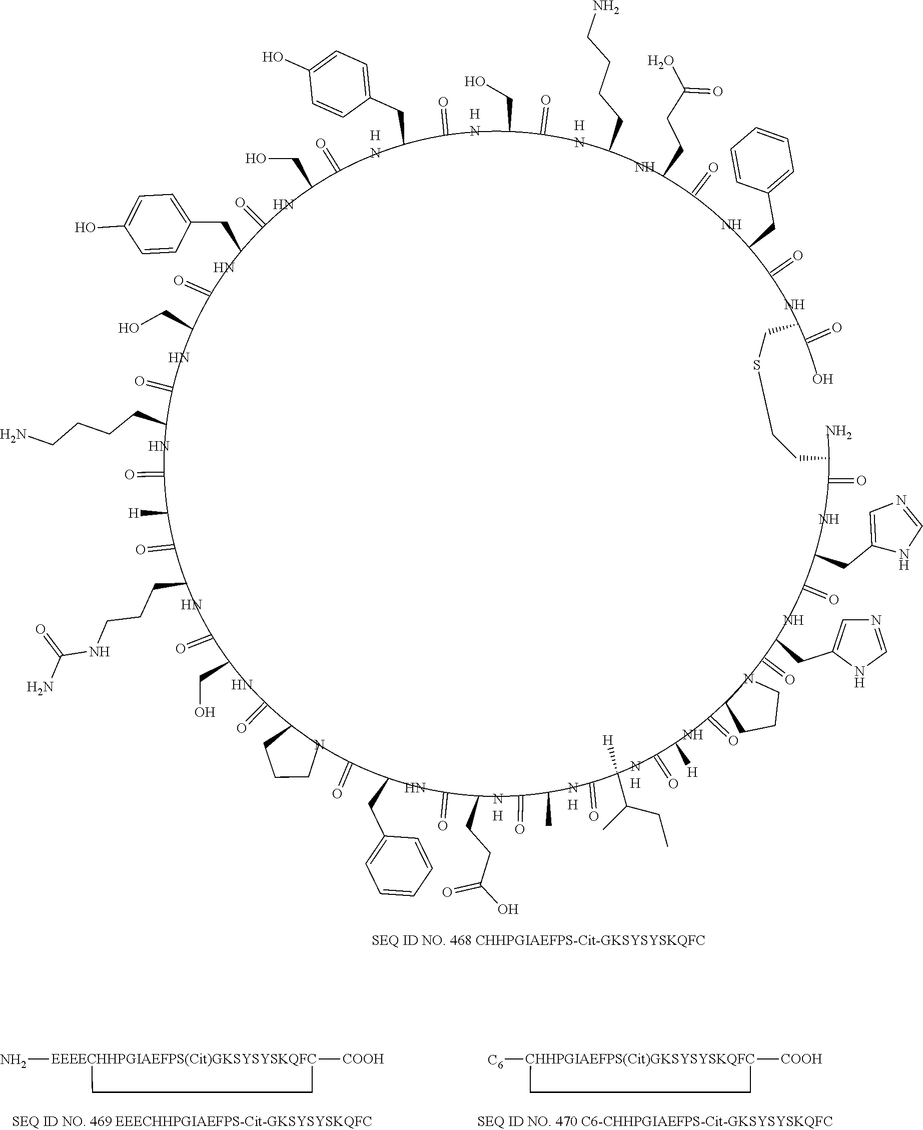

In certain embodiments, the ACPA Targeting Ligand is a nanoparticle prepared according to Khatri, S. et al “Cyclic Citrullinated Peptide Aptamer Treatment Attenuates Collagen-Induced Arthritis” Biomacromolecules 2022, 23, 2126-2137.

In certain embodiments, the ACPA Targeting Ligand is selected from SEQ ID NO: 471-490 (Khatri, S. et al. “Antibodies to synthetic citrullinated peptide epitope correlate with disease activity and flares in rheumatoid arthritis” PLoS ONE 15 (4): e0232010).

SEQ ID NO: 471
HHPGIAEFPS(Cit)GKSSSYSKQF
SEQ ID NO: 472
HHPGIAEFPS(Cit)GKSYSYS KQF
SEQ ID NO: 473
HGP GIA EFP S(Cit)G PSY SYS KQF
SEQ ID NO: 474
AEGGGV(Cit)GPRVVE
SEQ ID NO: 475
ASSGGV(Cit)GPRIVE
SEQ ID NO: 476
KDLLPS(Cit)D(Cit)QHLPLIK
SEQ ID NO: 477
QMRMELE(Cit)PGGNEIT(Cit)GGSTSYG
SEQ ID NO: 478
NVSPGT(Cit)(Cit)EYHTEK
SEQ ID NO: 479
ST(Cit)SVSSSSY(Cit)(Cit)MFGG
SEQ ID NO: 480
VYAT(Cit)SSAV(Cit)L(Cit)SSVP
SEQ ID NO: 481
A(Cit)TKQTA(Cit)KSTGGKAP
SEQ ID NO: 482
AA(Cit)KSAPSTGGVKKPH
SEQ ID NO: 483
Y(Cit)PGTVAL(Cit)EIKKYQKS
SEQ ID NO: 484
LI(Cit)KLPFQ(Cit)LV(Cit)EIAQDFK
SEQ ID NO: 485
LCAIHAK(Cit)VTIMPKDI
SEQ ID NO: 486
A(Cit)GLTG(Cit)PGDA
SEQ ID NO: 487
SHQEST(Cit)G(Cit)S(Cit)GRSGRSGS
SEQ ID NO: 488
T(Cit)GRS
SEQ ID NO: 489
T(Cit)G(Cit)S
SEQ ID NO: 490
TRG(Cit)S

In certain embodiments, the ACPA Targeting Ligand is selected from SEQ ID NO: 491-501 (Fernandes-Cerqueira et al. Targeting of anti-citrullinated protein/peptide antibodies in rheumatoid arthritis using peptides mimicking endogenously citrullinated fibrinogen antigens Arthritis Research & Therapy (2015) 17:155). In certain embodiments, the ACPA Targeting Ligand is a cyclized form of SEQ ID NO: 491-501. In certain embodiments, the ACPS Targeting Ligand is a linear or cyclic peptide with at least about 99%, 98%, 95%, 92%, 90%, 85%, or 80% sequence homology with a peptide selected from SEQ ID NO: 491-501. In certain embodiments, the ACPS Targeting Ligand is a linear or cyclic peptide of SEQ ID NO 491-501 with one, two, three, or four amino acids replaced with citrulline.

SEQ ID NO: 491
HHP GIA EFP SRG KSS SYS KQF
SEQ ID NO: 492
HHP GIA EFP SXG KSS SYS KQF (wherein X is citrulline)
SEQ ID NO: 493
SKQ FTS STS YNR GDS TFE SKS
SEQ ID NO: 494
SKQ FTS STS YNX GDS TFE SKS (wherein X is citrulline)
SEQ ID NO: 495
APP PIS GGG YRA RPA KAA AT
SEQ ID NO: 496
APP PIS GGG YXA RPA KAA AT (wherein X is citrulline)
SEQ ID NO: 497
APP PIS GGG YRA XPA KAA AT (wherein X is citrulline)
SEQ ID NO: 498
cyclo-CHHP GIA EFP SXG KSS SYS KQF (wherein X is citrulline)
SEQ ID NO: 499
GIA EFP SXG KSS SYS (wherein X is citrulline)
SEQ ID NO: 500
A EFP SXG KSS S (wherein X is citrulline)

### Desmoglein-2 (DSG-2) Autoantibodies

In certain embodiments, the Extracellular Target Protein is a desmoglein-2 (DSG-2) autoantibody. In certain embodiments, a Targeted Protein Degrader that degrades autoantibodies to DSG-2 can be used in the treatment of diseases including but not limited to Arrhythmogenic Right Ventricular Cardiomyopathy or post-COVID-19 cardiac syndrome.

In certain embodiments, the DSG-2 autoantibody Targeting Ligand is a DSG-2 fusion protein wherein the fusion protein comprises a whole or a portion of a DSG-2 protein and a whole or a portion of an immunoglobulin protein (as described in WO 2022/132854A1).

In certain embodiments, the DSG-2 autoantibody Targeting Ligand is a DSG-2 fusion protein wherein the fusion protein comprises the extracellular region of DSG-2 protein (SEQ ID NO: 501) and a whole or a portion of an immunoglobulin protein.

SEQ ID NO: 501
Ala Trp Ile Thr Ala Pro Val Ala Leu Arg Glu Gly Glu Asp Leu Ser Lys Lys Asn
Pro Ile Ala Lys Ile His Ser Asp Leu Ala Glu Glu Arg Gly Leu Lys Ile Thr Tyr Lys Tyr Thr Gly
Lys Gly Ile Thr Glu Pro Pro Phe Gly Ile Phe Val Phe Asn Lys Asp Thr Gly Glu Leu Asn Val Thr
Ser Ile Leu Asp Arg Glu Glu Thr Pro Phe Phe Leu Leu Thr Gly Tyr Ala Leu Asp Ala Arg Gly
Asn Asn Val Glu Lys Pro Leu Glu Leu Arg Ile Lys Val Leu Asp Ile Asn Asp Asn Glu Pro Val
Phe Thr Gln Asp Val Phe Val Gly Ser Val Glu Glu Leu Ser Ala Ala His Thr Leu Val Met Lys Ile
Asn Ala Thr Asp Ala Asp Glu Pro Asn Thr Leu Asn Ser Lys Ile Ser Tyr Arg Ile Val Ser Leu Glu
Pro Ala Tyr Pro Pro Val Phe Tyr Leu Asn Lys Asp Thr Gly Glu Ile Tyr Thr Thr Ser Val Thr Leu
Asp Arg Glu Glu His Ser Ser Tyr Thr Leu Thr Val Glu Ala Arg Asp Gly Asn Gly Glu Val Thr
Asp Lys Pro Val Lys Gln Ala Gln Val Gln Ile Arg Ile Leu Asp Val Asn Asp Asn Ile Pro Val Val
Glu Asn Lys Val Leu Glu Gly Met Val Glu Glu Asn Gln Val Asn Val Glu Val Thr Arg Ile Lys
Val Phe Asp Ala Asp Glu Ile Gly Ser Asp Asn Trp Leu Ala Asn Phe Thr Phe Ala Ser Gly Asn
Glu Gly Gly Tyr Phe His Ile Glu Thr Asp Ala Gln Thr Asn Glu Gly Ile Val Thr Leu Ile Lys Glu
Val Asp Tyr Glu Glu Met Lys Asn Leu Asp Phe Ser Val Ile Val Ala Asn Lys Ala Ala Phe His Lys
Ser Ile Arg Ser Lys Tyr Lys Pro Thr Pro Ile Pro Ile Lys Val Lys Val Lys Asn Val Lys Glu Gly Ile
His Phe Lys Ser Ser Val Ile Ser Ile Tyr Val Ser Glu Ser Met Asp Arg Ser Ser Lys Gly Gln Ile Ile
Gly Asn Phe Gln Ala Phe Asp Glu Asp Thr Gly Leu Pro Ala His Ala Arg Tyr Val Lys Leu Glu
Asp Arg Asp Asn Trp Ile Ser Val Asp Ser Val Thr Ser Glu Ile Lys Leu Ala Lys Leu Pro Asp Phe
Glu Ser Arg Tyr Val Gln Asn Gly Thr Tyr Thr Val Lys Ile Val Ala Ile Ser Glu Asp Tyr Pro Arg
Lys Thr Ile Thr Gly Thr Val Leu Ile Asn Val Glu Asp Ile Asn Asp Asn Cys Pro Thr Leu Ile Glu
Pro Val Gln Thr Ile Cys His Asp Ala Glu Tyr Val Asn Val Thr Ala Glu Asp Leu Asp Gly His Pro
Asn Ser Gly Pro Phe Ser Phe Ser Val Ile Asp Lys Pro Pro Gly Met Ala Glu Lys Trp Lys Ile Ala
Arg Gln Glu Ser Thr Ser Val Leu Leu Gln Gln Ser Glu Lys Lys Leu Gly Arg Ser Glu Ile Gln Phe
Leu Ile Ser Asp Asn Gln Gly Phe Ser Cys Pro Glu Lys Gln Val Leu Thr Leu Thr Val Cys Glu Cys
Leu His Gly Ser Gly Cys Arg Glu Ala Gln His Asp Ser Tyr Val Gly

In certain embodiments, the DSG-2 autoantibody Targeting Ligand is a DSG-2 fusion protein wherein the fusion protein comprises the extracellular cadherin domain 1 (EC1), extracellular cadherin domain 2 (EC2), extracellular cadherin domain 3 (EC3), extracellular cadherin domain 4 (EC4), and/or extracellular anchor domain (EA) of DSG-2 protein and a whole or a portion of an immunoglobulin protein.

### ApoE Targeting Ligands

In certain embodiments the Extracellular Protein Targeting Ligand binds to apolipoprotein E (ApoE). ApoE is a multifunctional protein with central roles in lipid metabolism, neurobiology, and neurodegenerative diseases. It has three major isoforms (apoE2, apoE3, and apoE4) with different effects on lipid and neuronal homeostasis. It is a protein involved in the metabolism of fats in the body of mammals.

In certain embodiments the ApoE Targeting Ligand is a nanobody that is identified as “sdAb-ApoE-Nbx”. For example in certain embodiments the ApoE Targeting Ligand is nanobody sdAb-ApoE-Nbx which is purchased from Gulliver Biomed.

In other aspects the ApoE Targeting Ligand is a lipid nanoparticle or a component of a lipid nanoparticle (LNP). For example, in certain embodiments the ApoE Targeting Ligand is a LNP which endogenously desorpts PEG lipids and enables the subsequent binding of ApoE. Non-limiting examples of LNPs are described in the paper by Dillard et al. titled “On the Mechanism of Tissue-Specific mRNA Delivery by Selective Organ Targeting Nanoparticles.” PNAS 2021, 118, 52. In certain embodiments the LNP is mDLNP as described in the paper by Dillard et al. In other embodiments the LNP is DLin-MC3-DMA LNP as described in the paper by Dillard et al.

In other aspects the ApoE Targeting Ligand is an antibody or antibody fragment selected from ab183597, ab52607, ab51015, ab183596, ab271944, ab227993, ab1907, ab171357, ab184845, ab245999, ab271843, ab215086, ab1906, ab195855, ab24139, ab83115, ab196194, ab196463, ab307386, ab307392, ab302567, ab24274, ab55210, ab32897, ab280330, ab302580, ab300743, ab242844, ab242598, ab244739, ab245008, ab241856, ab27613, ab312030, ab313170, ab312507, ab312961, ab310501, ab311685, ab310350, ab308786, ab308649, ab306401, ab310867, ab303051, ab306402, ab303052, ab306400, ab310931, ab303050, ab123749, ab226314, ab267976, ab108813, ab279714, ab279719, ab169861, ab27543, ab85975, ab85976, ab286161, ab123764, ab 123766, ab50242, ab233623, and ab244096.

In other aspects the ApoE Targeting Ligand binds to ApoE4. Non-limiting examples of antibodies or antibody fragments that bind to ApoE4 include: ab279714, ab279719, ab169861, ab85975, ab85976, and ab123766.

In other aspects the ApoE Targeting Ligand binds to ApoE3. Non-limiting examples of antibodies or antibody fragments that bind to ApoE3 include: ab27543, ab286161, ab 123764, and ab50242.

In certain embodiments the ApoE Targeting Ligand is a sequence of greater than 80%, 85%, 90%, 91%, 92%, 93%, 94%, 95%, 96%, 97%, 98%, or 99% homology to an ApoE Targeting antibody or antibody fragment described herein. In certain aspects the ApoE Targeting Ligand can be purchased from abcam.

### Additional Extracellular Protein Targets

In certain embodiments, the Extracellular Protein Targeting Ligand binds a protein target selected from the group consisting of 1-40-β-amyloid, 5′-nucleotidase, activated F9, F10, activin receptor-like kinase 1, alpha-fetoprotein, amyloid, angiopoietin 2, angiopoietin 3, anthrax toxin, AOC3, AOC3 (VAP-1), Bacillus anthracis anthrax, BAFF, beta amyloid, c-Met, C₁s, C242 antigen, C5, CA-125, calcitonin, calcitonin gene-related peptide, calcitonin gene-related peptide alpha, Canis lupus familiaris IL31, carbonic anhydrase 9 (CA-IX), CEA, CEA-related antigen, CEACAM5, CFD, CGRP, clumping factor A, coagulation factor III, complement C5a, CSF1, MCSF, CSF2, dabigatran, E. coli shiga toxin type-1, E. coli shiga toxin type-2, EGFL7, EGFR, endotoxin, episialin, FGF 23, fibrin II, beta chain, fibronectin extra domain —B, folate hydrolase, GDF8, gelatinase B, GMCSF, growth differentiation factor 8, hemagglutinin, hemagglutinin HA, HGF, HIV-1, HNGF, Hsp90, human beta-amyloid, human scatter factor receptor kinase, human TNF, IFN-gamma, IFN-γ, IgE, IgE Fc region, IGF1, IGF2, IGHE, IL 17A, IL 17A and IL 17F, IL 20, IL-1, IL-12, IL-23, IL-13, IL-17, IL-1B, IL-22, IL-4, IL-5, IL-6, IL17A and IL17F, ILIA, IL2, IL23, IL23A, IL31RA, IL6, IL6R, IL9, ILGF2, Influenza A hemagglutinin, influenza A virus hemagglutinin, influenza A virus hemagglutinin HA, interferon gamma, interferon gamma-induced protein, interleukin 1 alpha, interleukin 13, interleukin 17 alpha, interleukin 17 alpha, TNF, interleukin 17A, kallikrein, LOXL2, LRRC15, LTA, MASP-2, MCP-1, MIF, MST1R (aka RON), MUC1, myostatin, NACP, NCA-90 (granulocyte antigen), neural apoptosis-regulated proteinase 1, NGF, NOGO-A, Notch 1, NRP1, oxLDL, PCSK9, PD-L1, phosphatidylserine; RANKL, RGMA, root plate-specific spondin 3, RTN4, sclerostin, SDC1, serum amyloid A protein, serum amyloid P component, SOST, Staphylococcus aureus alpha toxin, tau protein, TFPI, TGF beta 1, TGF beta 2, TROP-2, TSLP, VEGF-A, VEGF-A and Ang-2, VEGFA, and VWF.

In certain embodiments, the Extracellular Protein Targeting Ligand is an antibody ligand selected from Abagovomab, Abrezekimab, Adalimumab, Aducanumab, Afasevikumab; Afelimomab, Alirocumab, Altumomab, Altumomab pentetate, Andecaliximab, Anrukinzumab, Arcitumomab, Ascrinvacumab, Atezolizumab, Atidortoxumab, Atinumab, Avelumab, Bapineuzumab, Bavituximab, Belimumab, Bermekimab, Besilesomab, Bevacizumab, Biciromab, Bimekizumab, Birtamimab, Blosozumab, Bococizumab, Brazikumab, Briakinumab, Brodalumab, Brolucizumab, Brontictuzumab, Burosumab, Cabiralizumab, Canakinumab, Cantuzumab; Cantuzumab ravtansine, Caplacizumab, Carlumab, Cergutuzumab, Cergutuzumab amunaleukin, Certolizumab, Certolizumab pegol, Cibisatamab, Clazakizumab, Clivatuzumab, Clivatuzumab tetraxetan, Concizumab, Crenezumab, Dectrekumab, Denosumab, Dezamizumab, Diridavumab, Domagrozumab, Dorlimomab, Dorlimomab aritox, Durvalumab, Dusigitumab, Eculizumab, Edobacomab, Efungumab, Eldelumab, Elezanumab, Elsilimomab, Emactuzumab, Emapalumab; Emicizumab, Enokizumab, Epitumomab, Epitumomab cituxetan, Eptinezumab, Erenumab, Evinacumab, Evolocumab, Faricimab, Fasinumab, Fezakinumab, Ficlatuzumab, Firivumab, Fletikumab, Fontolizumab, Fremanezumab, Fresolimumab, Frovocimab, Frunevetmab, Fulranumab, Galcanezumab, Gantenerumab, Gatipotuzumab, Gedivumab, Gevokizumab, Gimsilumab, Girentuximab, Golimumab, Gosuranemab, Guselkumab, Idarucizumab, Igovomab; Imalumab, Indatuximab, Indatuximab ravtansine, Infliximab, Istiratumab, Ixekizumab, Labetuzumab, Lacnotuzumab, Lampalizumab, Lanadelumab, Landogrozumab, Lebrikizumab, Lemalesomab, Lendalizumab, Lenzilumab, Lerdelimumab, Lesofavumab, Ligelizumab, Lodelcizumab, Lokivetmab, Lutikizumab, Marstacimab, Mepolizumab, Metelimumab, Mirikizumab, Nacolomab, Nacolomab tafenatox, Namilumab, Narnatumab, Navivumab, Naxitamab, Nebacumab, Nemolizumab, NEOD, Nerelimomab, Nesvacumab, Netakimab, Nofetumomab, Nofetumomab merpentan, Obiltoxaximab, Oleclumab, Olendalizumab; Olokizumab, Omalizumab, OMS, Onartuzumab, Oregovomab, Orticumab, Otilimab; Ozanezumab, Ozoralizumab, Parsatuzumab, Pascolizumab, Pasotuxizumab, Pateclizumab, Pemtumomab, Perakizumab, Pexelizumab, Placulumab, Ponezumab, Prasinezumab, Pritoxaximab, Quilizumab, Radretumab, Ralpancizumab, Ranevetmab, Ranibizumab; Ravulizumab, Raxibacumab, REGN-EB, Remtolumab, Reslizumab, Rilotumumab, Risankizumab, Romilkimab, Romosozumab, Rontalizumab, Rosmantuzumab, Sacituzumab, Sacituzumab govitecan, Samrotamab, Samrotamab vedotin, Sarilumab, Secukinumab, Setoxaximab, Setrusumab, Sifalimumab, Siltuximab, Simtuzumab, Sirukumab, Sofituzumab, Sofituzumab, Sofituzumab vedotin, Solanezumab, Sontuzumab, Stamulumab, Sulesomab, Sutimlimab, Suvizumab, Suvratoxumab, Tabalumab, Tacatuzumab, Tacatuzumab tetraxetan, Talizumab, Tanezumab, Tefibazumab, Telimomab, Telimomab aritox, Tesidolumab, Tezepelumab, Tibulizumab, Tildrakizumab, Timolumab, Tisotumab, Tisotumab vedotin, Tralokinumab, Trevogrumab, Urtoxazumab, Ustekinumab, Vanucizumab, Vapaliximab, Varisacumab, Vepalimomab, Vesencumab, Vobarilizumab, Vunakizumab, and Xentuzumab.

In certain embodiments, the Extracellular Protein Targeting Ligand binds a protein target selected from the group consisting of TNFa, HER2, EGFR, HER3, VEGFR, CD20, CD 19, CD22, anb3 integrin, CEA, CXCR4, MUC1, LCAM1, EphA2, PD-1, PD-L1, TIGIT, TIM3, CTLA4, VISTA, Notch receptors, EGF, c-MET, CCL2, CCR2, Frizzled receptors, Wnt, LRP5/6, CSF-1R, SIRPa, CD38, CD73, TGF-b, Bombesin R, CAIX, CD13, CD44v6, Emmprin, Endoglin, EpCAM, FAP-a, Folate R, GRP78, IGF-1R, Matriptase, Mesothelin, sMET/HGFR, MT1-MMP, MT6-MMP, PSCA, PSMA, Tn antigen, and uPAR, TSHRa, Myelin oligodendrocyte glycoprotein (MOG), AChR-al, noncollagen domain 1 of the a3 chain of type IV collagen (a3NCl), ADAMTS13, Desmoglein-1/3, or GPIb/IX, GPIIb/IIIa, GPIa/IIa, NMDA receptor, glutamic acid decarboxylase (GAD), amphiphysin and gangliosides GM1, GD3, GQ1B, LILRB1, LILR^{B2}; VEGF-R, CXCR4, CXCL12, CSF1, CD47, aggregated light chain or aggregated transthyretin.

In certain embodiments, the Extracellular Protein Targeting Ligand binds an antibody that binds to TSHRa, MOG, AChR-al, noncollagen domain 1 of the a3 chain of type IV collagen (a3NCl), ADAMTS13, Desmoglein-1/3, or GPIb/IX, GPIIb/IIIa, GPIa/IIa, NMDA receptor, glutamic acid decarboxylase (GAD), amphiphysin, or gangliosides GM1, GD3 or GQ1B.

In certain embodiments, the Extracellular Protein Targeting Ligand binds SIRPa, CCR2, CSF1R, LILRB1, LILR^{B2}, VEGF-R, or CXCR4. In other embodiments, the target protein associated with TAMs comprises CCL2, CXCL12, CSF1 or CD47.

In certain embodiments, the Extracellular Protein Targeting Ligand binds a protein that is upregulated in cancer or involved in cancer progression. In some embodiments, the target protein upregulated in cancer or involved in cancer progression comprises HER2, EGFR, HER3, VEGFR CD20, CD19, CD22, anb3 integrin, CEA, CXCR4, MUC1, LCAM1, EphA2, PD-1, PD-L1, TIGIT, TIM3, CTLA4, VISTA, Notch receptors, EGF, c-MET, CCL2, CCR2, Frizzled receptors, Wnt, LRP5/6, CSF1R, SIRPa, CD38, CD73, or TGF-b.

In certain embodiments, the Extracellular Protein Targeting Ligand binds an autoantibody of an autoimmune disease. In some embodiments, the target protein is an autoantigen in an autoimmune disease. In some embodiments, the autoimmune disease is selected from Graves' Disease, Myelin oligodendrocyte glycoprotein antibody- associated disease (MOGAD), Myasthenia Gravis, Anti-GBM Disease, Immune Thrombotic Thrombocytopeniarpura, Acquired Pemphigus Vulgaris, Immune Thrombocytopenia, autoimmune encephalitis, Guillain-Barre Syndrome, and Membranous Nephropathy.

In certain embodiments, the Extracellular Protein Targeting Ligand is a ligand that binds an autoantibody which itself binds disintegrin and metalloproteinase with a thrombospondin type 1 motif, member 13 (ADAMTS13), steroidogenic cytochrome P450 enzyme 21-hydroxylase, N-methyl-d-aspartate-(NMDA)-receptor, erythrocytes, anti-smooth muscle antibodies (ASMAs), actin, platelet, signal recognition particle (SRP), 3-hydroxy-3-methyl-glutaryl-coenzyme A reductase (HMGCR), myosin, sperm, amylase alpha2, type XVII collagen (col 17), kallikrein 13, type VII collagen (col7), myeloperoxidase (MPO), type IV collagen, proteinase 3 (PR3), thyrotropin receptor (TSHR), thyroglobulin, thyroid peroxidase (TPO), thyroglobulin, thyroid peroxidase (TPO), platelets, myeloperoxidase (MPO), muscle nicotinic acetylcholine receptors, muscle-specific kinase (MuSK), low-density lipoprotein receptor protein 4 (LRP4), myosin, beta-1 adrenergic receptor, adenine-nucleotide translocase, aquaporin-4, myelin oligodendrocyte glycoprotein (MOG), heat shock protein 90 (HSP90), heat shock protein A5 (HSPA5), desmoglein-3, parietal cells, mitochondria, phospholipase A2 receptor (PLA2R), thrombospondin type 1 domain-containing 7A (THSD7A), cyclic citrullinated proteins, RNA binding proteins (Ros), La, double-stranded DNA (dsDNA), angiotensin II type 1 receptor (ATIR), endothelin-1 type A receptor (ETAR), insulin, glutamic acid decarboxylase or protein tyrosine phosphatase.

In some embodiments, the autoantibody in the autoimmune disease is an antibody binding to TSHRa, Myelin oligodendrocyte protein (MOG), AChR-al, noncollagen domain 1 of the a3 chain of type IV collagen (a3NCl), ADAMTS13, Desmoglein-1/3, GPIb/IX, GPIIb/IIIa, GPIa/IIa, NMDA receptor, glutamic acid decarboxylase (GAD), amphiphysin, or gangliosides GM1, GD3 or GQIB.

In some embodiments, the Extracellular Protein Targeting Ligand binds a protein that is upregulated or expressed in a neurodegenerative disease. In some embodiments, the target protein upregulated or expressed in a neurodegenerative disease is alpha-synuclein, amyloid beta or complement cascade component.

In some embodiments, the Extracellular Protein Targeting Ligand binds a protein that is upregulated in amyloidosis. In some embodiments, the amyloidosis can be systemic amyloidosis. In other embodiments, the amyloidosis can be localized amyloidosis. In some embodiments, the protein upregulated in systemic amyloidosis can be transthyretin.

In some embodiments, the Extracellular Protein Targeting Ligand binds an immune checkpoint protein. In some embodiments, the target protein comprises a cancer antigen. In certain embodiments, the cancer antigen comprises HER2, EGFR, CDCP1, CD38, IGF1R, MMP14, and TROP2.

In some embodiments, the Extracellular Protein Targeting Ligand binds an immunomodulatory protein. In certain embodiments, the immunomodulatory protein comprises PD-L1, PD-1, CTLA-4, B7-H3, B7-H4, LAG3, NKG2D, TIM-3, VISTA, CD39, CD73 (NT5E), A2AR, SIGLEC7, and SIGLEC15. In some embodiments, the target protein comprises a B cell antigen. In some exemplary embodiments, the B cell antigen comprises CD 19 and CD20.

In certain embodiments, the Extracellular Protein Targeting Ligand binds a soluble target protein. In some embodiments, the soluble target protein comprises an inflammatory cytokine, a growth factor (GF), a toxic enzyme, a target associated with metabolic diseases, a neuronal aggregate, or an autoantibody. In certain embodiments, the inflammatory cytokine comprises lymphotoxin, interleukin-1 (IL-1), IL-2, IL-5, IL-6, IL-12, IL-13, IL-17, IL-18, IL-23, tumor necrosis factor alpha (TNF-α), interferon gamma (IFNy), and granulocyte-macrophage colony stimulating factor (GM-CSF). In certain embodiments, the growth factor comprises EGF, FGF, NGF, PDGF, VEGF, IGF, GMCSF, GCSF, TGF, RANK-L, erythropieitn, TPO, BMP, HGF, GDF, neurotrophins, MSF, SGF, GDF, and an isoform thereof. In certain embodiments, the toxic enzyme comprises a protein arginine deiminase 1 (PAD1), PAD2, PAD3, PAD4, and PAD6, leucocidin, hemolysin, coagulase, treptokinase, hyaluronidase. In certain embodiments, the toxic enzyme comprises PAD2 or PAD4. In some embodiments, the neuronal aggregate comprises Ab, TTR, a-synuclein, TAO, and prion. In certain embodiments, the autoantibody comprises IgA, IgE,
IgG, IgMand IgD.

In certain embodiments, the Extracellular Protein Targeting Ligand binds a growth factor, a cytokine, a chemokine, a hormone, a neurotransmitter, a capsid, a soluble receptor, an extracellular secreted protein, an antibody, a lipoprotein, an exosome, a virus, a cell, or a plasma membrane protein, wherein the bifunctional compounds of the invention can then be used to direct the extracellular target molecule to lysosomes for degradation. Examples of such target molecules which can be directed for degradation using the bifunctional compound of the invention include, but are not limited to, LDL (ApoB), Lp (a), ApoCIII, ANGPTL3, ANGPTL4, ANGPTL8, Factor 11, GDF15, LPL, PCSK9, IL1 B, IL17, Complement Factor B, Complement Factor D, MPO, IgE, IL7, IL 12A, IL23, TNFA, CXCR4, MAPT, FHR3, TIMP1, Apelin, BMP6, BMP9/GDF2, CSF1, EPO, IL5, MFGE8, TSLP, TSP, C5, CXCL10, FGF²³, IGF1, IL10, IL13, IL2, IL6, VEGFA, NKG2D, ZNFR3, ADA2, suPAR, TGF-β1, IL4 receptor, sToll receptor, histamine, Tau, proglanulin, Alpha-synuclein, toxins, venoms, HBV soluble antigen, viral antigens, prion protein, scFV, AAV and anti-AAV antibodies. In certain embodimenst, the extracellular target molecules which can be directed for degradation using the bifunctional compound of the invention are PCSK9 and FHR3.

In certain embodiments, the Extracellular Protein Targeting Ligand binds a protein selected from proprotein convertase subtilisin/kexin type 9 (PCSK9), tumor necrosis factor receptor 1 (TNFR^{E}), interleukin-1 receptor (ILIR), low density lipoproteins, very low density lipoproteins, chylomicrons, apolipoprotein B (ApoB), lipoprotein (a) (Lp (a)), apolipoprotein C3 (ApoCIII), angiopoietin-like 3 (ANGPTL3), angiopoietin-like 4 (ANGPTL4), angiopoietin-like 8 (ANGPTL8), Factor 11, growth differentiation factor 15 (GDF15), lipoprotein lipase (LPL), interleukin 1-beta (IL 113), interleukin 17 (IL 17), complement Factor B, complement Factor D, myeloperoxidase (MPO), immunoglobulin A (IgA), immunoglobulin E (IgE), programmed cell death protein 1 (PD-1), programmed death-ligand 1 (PD-L1), interleukin 7 (IL7), interleukin 12A (IL12A), interleukin 23 (IL23), tumor necrosis factor A (TNFA), microtubule associated protein tau (MAPT), complement factor H-related protein 3 (FHR3), tissue inhibitor of metalloproteinases 1 (TIMP1), Apelin, bone morphogenetic protein 6 (BMP6), bone morphogenetic protein 9/growth differentiation factor 2 (BMP9/GDF2), colony stimulating factor 1 receptor (CSF1), erythropoietin (EPO), interleukin 5 (IL5), milk fat globule-EGF Factor 8 protein (MFGE8), thymic stromal lymphopoietin (TSLP), thrombospondin (TSP), complement component 5 (C5), C—X—C motif chemokine ligand 10 (CXCL10), fibroblast growth factor 23 (FGF²³), insulin-like growth factor 1 (IGF1), interleukin 10 (IL 10), interleukin 13 (IL 13), interleukin 2 (IL2), interleukin 6 (IL6), vascular endothelial growth factor A (VEGF-A), adenosine deaminase 2 (ADA2), soluble urokinase-type plasminogen activator receptor (suPAR), transforming growth factor beta 1 (TGF-pi), progranulin, alpha-synuclein, a toxin, a venom, an HBV soluble antigen, a viral antigen, a prion protein, a scFv, an AAV, and an anti-AAV antibody.

### IV. Pharmaceutical Compositions and Dosage Forms for the Extracellular Degrading Compounds of the Present Invention

A compound of the present invention or a pharmaceutically acceptable salt, solvate or prodrug thereof as disclosed herein can be administered as a neat chemical, but is more typically administered as a pharmaceutical composition that includes an effective amount for a host, typically a human, in need of such treatment to treat a disorder mediated by the target extracellular protein, as described herein or otherwise well-known for that extracellular protein. The Extracellular Protein Degraders of the present invention can be administered in any manner that allows the degrader to bind to the Extracellular Protein, typically in the blood stream, and carry it to the mannose 6-phosphate receptor bearing cells for endocytosis and degradation. As such, examples of methods to deliver the degraders of the present invention include, but are not limited to, oral, intravenous, sublingual, subcutaneous, parenteral, buccal, rectal, intra-aortal, intracranial, subdermal, transdermal, controlled drug delivery, intramuscular, or transnasal, or by other means, in dosage unit formulations containing one or more conventional pharmaceutically acceptable carriers, as appropriate. In certain embodiments, the degrader is provided in a liquid dosage form, a solid dosage form, a gel, particle, etc.

In certain embodiments the compound of the present invention is administered subcutaneously. Typically, the compound will be formulated in a liquid dosage form for subcutaneous injection, such as a buffered solution. Non-limiting examples of solutions for subcutaneous injection include phosphate buffered solution and saline buffered solution. In certain embodiments the solution is buffered with multiple salts.

In certain embodiments the compound of the present invention is administered intravaneously. Typically, the compound will be formulated in a liquid dosage form for intravaneous injection, such as a buffered solution. Non-limiting examples of solutions for intravaneous injection include phosphate buffered solution and saline buffered solution. In certain embodiments the solution is buffered with multiple salts.

Therefore, the disclosure provides pharmaceutical compositions comprising an effective amount of degrading compound or its pharmaceutically acceptable salt together with at least one pharmaceutically acceptable carrier for any appropriate use thereof. The pharmaceutical composition may contain a compound or salt as the only active agent, or, in an alternative embodiment, the compound and at least one additional active agent.

The term “pharmaceutically acceptable salt” as used herein refers to a salt of the described compound which is, within the scope of sound medical judgment, suitable for administration to a host such as a human without undue toxicity, irritation, allergic response, and the like, commensurate with a reasonable benefit/risk ratio, and effective for its intended use. Thus, the term “pharmaceutically acceptable salt” refers to the relatively non-toxic, inorganic and organic acid addition salts of the presently disclosed compounds. These salts can be prepared during the final isolation and purification of the compounds or by separately reacting the purified compound in its free form with a suitable organic or inorganic acid and then isolating the salt thus formed. Basic compounds are capable of forming a wide variety of different salts with various inorganic and organic acids. Acid addition salts of the basic compounds are prepared by contacting the free base form with a sufficient amount of the desired acid to produce the salt in the conventional manner. The free base form can be regenerated by contacting the salt form with a base and isolating the free base in the conventional manner. The free base forms may differ from their respective salt forms in certain physical properties such as solubility in polar solvents. Pharmaceutically acceptable base addition salts may be formed with a metal or amine, such as alkali and alkaline earth metal hydroxide, or an organic amine. Examples of metals used as cations, include, but are not limited to, sodium, potassium, magnesium, calcium, and the like. Examples of suitable amines include, but are not limited to, N,N′-dibenzylethylenediamine, chloroprocaine, choline, diethanolamine, ethylenediamine, N-methylglucamine, and procaine. The base addition salts of acidic compounds are prepared by contacting the free acid form with a sufficient amount of the desired base to produce the salt in the conventional manner. The free acid form can be regenerated by contacting the salt form with an acid and isolating the free acid in a conventional manner. The free acid forms may differ from their respective salt forms somewhat in certain physical properties such as solubility in polar solvents.

Salts can be prepared from inorganic acids sulfate, pyrosulfate, bisulfate, sulfite, bisulfite, nitrate, phosphate, monohydrogenphosphate, dihydrogenphosphate, metaphosphate, pyrophosphate, chloride, bromide, iodide, nitric, phosphoric, sulfuric, hydrobromic, hydriodic, phosphorus, and the like. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, laurylsulphonate and isethionate salts, and the like. Salts can also be prepared from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. and the like. Representative salts include acetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Pharmaceutically acceptable salts can include cations based on the alkali and alkaline earth metals, such as sodium, lithium, potassium, calcium, magnesium and the like, as well as non-toxic ammonium, quaternary ammonium, and amine cations including, but not limited to, ammonium, tetramethylammonium, tetraethylammonium, methylamine, dimethylamine, trimethylamine, triethylamine, ethylamine, and the like. Also contemplated are the salts of amino acids such as arginate, gluconate, galacturonate, and the like. See, for example, Berge et al., J. Pharm. Sci., 1977, 66, 1-19, which is incorporated herein by reference.

Any dosage form can be used that achieves the desired results. In certain embodiments the pharmaceutical composition is in a dosage form that contains from about 0.1 mg to about 1500 mg, from about 10 mg to about 1000 mg, from about 100 mg to about 800 mg, or from about 200 mg to about 600 mg of the active compound and optionally from about 0.1 mg to about 1500 mg, from about 10 mg to about 1000 mg, from about 100 mg to about 800 mg, or from about 200 mg to about 600 mg of an additional active agent in a unit dosage form. Examples are dosage forms with at least 0.1, 1, 5, 10, 25, 50, 100, 200, 250, 300, 400, 500, 600, 700, or 750 mg of active compound, or its salt.

In certain embodiments the dose ranges from about 0.01-100 mg/kg of patient bodyweight, for example about 0.01 mg/kg, about 0.05 mg/kg, about 0.1 mg/kg, about 0.5 mg/kg, about 1 mg/kg, about 1.5 mg/kg, about 2 mg/kg, about 2.5 mg/kg, about 3 mg/kg, about 3.5 mg/kg, about 4 mg/kg, about 4.5 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, or about 100 mg/kg.

In some embodiments, compounds disclosed herein or used as described are administered once a day (QD), twice a day (BID), or three times a day (TID). In some embodiments, compounds disclosed herein or used as described are administered at least once a day for at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, at least 31 days, at least 35 days, at least 45 days, at least 60 days, at least 75 days, at least 90 days, at least 120 days, at least 150 days, at least 180 days, or longer.

In certain embodiments the compound of the present invention is administered once a day, twice a day, three times a day, or four times a day.

In certain embodiments, the compound of the present invention is administered once every other day or once every three, four, five, or six days. In certain embodiments, the compound of the present invention is administered once per week. In certain embodiments, the compound of the present invention is administered once every other week. In certain embodiments, the compound of the present invention is administered once per month. In certain embodiments, the compound of the present invention is administered once every other month or once every three months.

The pharmaceutical composition may be formulated as any pharmaceutically useful form, e.g., a pill, capsule, tablet, an injection or infusion solution, a syrup, an inhalation formulation, a suppository, a buccal or sublingual formulation, a parenteral formulation, or in a medical device. Some dosage forms, such as tablets and capsules, can be subdivided into suitably sized unit doses containing appropriate quantities of the active components, e.g., an effective amount to achieve the desired purpose.

Carriers include excipients and diluents and must be of sufficiently high purity and sufficiently low toxicity to render them suitable for administration to the patient being treated. The carrier can be inert or it can possess pharmaceutical benefits of its own. The amount of carrier employed in conjunction with the compound is sufficient to provide a practical quantity of material for administration per unit dose of the compound. If provided as in a liquid, it can be a solution or a suspension.

Representative carriers include phosphate buffered saline, water, solvent(s), diluents, pH modifying agents, preservatives, antioxidants, suspending agents, wetting agent, viscosity agents, tonicity agents, stabilizing agents, and combinations thereof. In some embodiments, the carrier is an aqueous carrier. Examples of aqueous carries include, but are not limited to, an aqueous solution or suspension, such as saline, plasma, bone marrow aspirate, buffers, such as Hank's Buffered Salt Solution (HBSS), HEPES (4-(2-hydroxyethyl)-1-piperazineethanesulfonic acid), Ringers buffer, Pro Visc®, diluted ProVisc®, Provisc® diluted with PBS, Krebs buffer, Dulbecco's PBS, normal PBS, sodium hyaluronate solution (HA, 5 mg/mL in PBS), citrate buffer, simulated body fluids, plasma platelet concentrate and tissue culture medium or an aqueous solution or suspension comprising an organic solvent. Acceptable solutions include, for example, water, Ringer's solution and isotonic sodium chloride solutions. The formulation may also be a sterile solution, suspension, or emulsion in a non-toxic diluent or solvent such as 1,3-butanediol.

Viscosity agents may be added to the pharmaceutical composition to increase the viscosity of the composition as desired. Examples of useful viscosity agents include, but are not limited to, hyaluronic acid, sodium hyaluronate, carbomers, polyacrylic acid, cellulosic derivatives, polycarbophil, polyvinylpyrrolidone, gelatin, dextin, polysaccharides, polyacrylamide, polyvinyl alcohol (including partially hydrolyzed polyvinyl acetate), polyvinyl acetate, derivatives thereof and mixtures thereof.

Solutions, suspensions, or emulsions for administration may be buffered with an effective amount necessary to maintain a pH suitable for the selected administration. Suitable buffers are well known by those skilled in the art. Some examples of useful buffers are acetate, borate, carbonate, citrate, and phosphate buffers. Solutions, suspensions, or emulsions for topical, for example, ocular administration may also contain one or more tonicity agents to adjust the isotonic range of the formulation. Suitable tonicity agents are well known in the art. Some examples include glycerin, mannitol, sorbitol, sodium chloride, and other electrolytes.

Classes of carriers include, but are not limited to binders, buffering agents, coloring agents, diluents, disintegrants, emulsifiers, flavorants, glidents, lubricants, preservatives, stabilizers, surfactants, tableting agents, and wetting agents. Some carriers may be listed in more than one class, for example vegetable oil may be used as a lubricant in some formulations and a diluent in others. Exemplary pharmaceutically acceptable carriers include sugars, starches, celluloses, powdered tragacanth, malt, gelatin; talc, and vegetable oils. Optional active agents may be included in a pharmaceutical composition, which do not substantially interfere with the activity of the compound of the present invention.

The pharmaceutical compositions/combinations can be formulated for oral administration. These compositions can contain any amount of active compound that achieves the desired result, for example between 0.1 and 99 weight % (wt. %) of the compound and usually at least about 5 wt. % of the compound. Some embodiments contain from about 25 wt. % to about 50 wt. % or from about 5 wt. % to about 75 wt. % of the compound. Enteric coated oral tablets may also be used to enhance bioavailability of the compounds for an oral route of administration.

Formulations suitable for rectal administration are typically presented as unit dose suppositories. These may be prepared by admixing the active compound with one or more conventional solid carriers, for example, cocoa butter, and then shaping the resulting mixture.

V. Treatment of Diseases with the Disclosed Extracellular Protein Degraders

The Target Proteins of the current invention may include, but are not limited to, immunoglobulins, cytokines, chemokines, growth factors, coagulation factors, extracellular matrix proteins and proteins involved in formation and/or degradation of the extracellular matrix, esterases, lipases, peptidases, convertases, among others. These proteins mediate a range of diseases that can be treated with an effective amount of the disclosed Extracellular Protein Degraders described herein.

### Immunoglobulins

1) Immunoglobulin A (IgA) aberrant expression mediates a range of autoimmune and immune-mediated disorders, including IgA nephropathy (also known as Berger's disease), celiac disease, Crohn's disease, Henoch-Sconiein purpura (HSP) (also known as IgA vasculitis), liner IgA bullous dermatosis, IgA pemphigus, dermatitis herpetiformis, inflammatory bowel disease (IBD), Sjögren's syndrome, ankylosing spondylitis, alcoholic liver cirrhosis, acquired immunodeficiency syndrome, IgA multiple myeloma, α-chain disease, IgA monoclonal gammopathy, monoclonal gammopathy of undetermined significance (MGUS), and linear IgA bullous dermatosis, among others.
2) Immunoglobulin G (IgG) mediates a range of autoimmune, infectious and metabolic diseases, including systemic fibroinflammatory disease. In addition, overexpression of IgG4 is associated with IgG4-related diseases, which generally include multiple organs, and disorders include type 1 autoimmune pancreatitis, interstitial nephritis, Riedel's thyroiditis, storiform fibrosis, Mikulicz's disease, Küttner's tumor, inflammatory pseudotumors (in various sites of the body), mediastinal fibrosis, retroperitoneal fibrosis (Ormond's disease), aortitis and periaortitis, proximal biliary strictures, idiopathic hypocomplementic tubulointerstitial nephritis, multifocal fibrosclerosis, pachymeningitis, pancreatic enlargement, tumefactive lesions, pericarditis, rheumatoid arthritis (RA), inflammatory bowel disease, multiple sclerosis, myasthenic gravis, ankylosing spondylitis, primary Sjögren's syndrome, psoriatic arthritis, and systemic lupus erythematosus (SLE), sclerosing cholangitis, and IgG monoclonal gammopathy, monoclonal gammopathy of undetermined significance (MGUS), among others.
3) Immunoglobulin E (IgE)-IgE is a strong mediator of allergic disease, including but not limited to, atopic asthma, allergic rhinitis, atopic dermatitis, IgE-mediated food allergy, IgE-mediated animal allergies, allergic conjunctivitis, allergic urticaria, anaphylactic shock, nasal polyposis, keratoconjunctivitis, mastocytosis, and eosinophilic gastrointestinal disease, bullous pemphigoid, chemotherapy induced hypersensitivity reaction, seasonal allergic rhinitis, interstitial cystitis, eosinophilic esophagitis, angioedema, acute interstitial nephritis, atopic eczema, eosinophilic bronchitis, chronic obstructive pulmonary disease, gastroenteritis, hyper-IgE syndrome (Job's Syndrome), IgE monoclonal gammopathy, and monoclonal gammopathy of undetermined significance (MGUS), among others.

### Cytokines/Chemokines

1) TNF-α mediates a number of disorders, including but not limited to rheumatoid arthritis, inflammatory bowel disease, graft-vs-host disease, ankylosing spondylitis, psoriasis, hidradenitis suppurativa, refractory asthma, systemic lupis erthyematosus, diabetes, and the induction of cachexia.
2) IL-2 mediates host versus graft rejection in transplants and autoimmune disorders, including, but not limited to, multiple sclerosis, idiopathic arthritis, iritis, anterior uveitis, IL-2 induced hypotension, psoriasis, and other autoimmune disorders
3) IL-1 mediates a number of auto-inflammatory and autoimmune disorders, including, but not limited to, Blau syndrome, cryopyrin- associated periodic syndromes, familial Mediterranean fever, Majeed syndrome; mevalonate kinase deficiency syndrome, pyogenic arthritis-pyoderma gangrenosum-acne syndrome, tumor necrosis factor receptor-associated periodic syndrome, Behçet's Disease, Sjogren's Syndrome, gout and chondrocalcinosis, periodic fever, aphthous stomatitis, pharyngitis, and cervical adenitis (or PFAPA) syndrome, rheumatoid arthritis, Type 2 diabetes mellitus, acute pericarditis, Chronic interstitial lung diseases (ILDs), and Still's disease amongst others.
4) IFN-γ mediates a wide range of autoimmune disorders, including, but not limited to rheumatoid arthritis, multiple sclerosis (MS), corneal transplant rejection, and various autoimmune skin diseases such as psoriasis, alopecia areata, vitiligo, acne vulgaris, and others.
5) IL-21 mediates a number of autoimmune disorders, including Sjögren's syndrome, systemic lupus erythematosus, type 1 diabetes, multiple sclerosis, rheumatoid arthritis, and inflammatory bowel disease.
6) IL-22 mediates a number of autoimmune disorders, including, but not limited to, graft versus host disease (GVHD), psoriasis, rheumatoid arthritis, atopic dermatitis, and asthma.
7) IL-10 has been implicated in tumor survival and protection against cytotoxic chemotherapeutic drugs.
8) IL-5 has been implicated in a number of allergic disorders, including, but not limited to, asthma, nasal polyposis, atopic dermatitis, eosinophilic esophagitis, hypereosinophilic syndrome, and Churg-Strauss syndrome.
9) IL-6 has been implicated in a number of inflammatory diseases and cancers, including, but not limited to, Castleman's disease, metastatic castration- associated prostate cancer, renal cell carcinoma, large-cell lung carcinoma, ovarian cancer, rheumatoid arthritis, asthma.
10) IL-8 has been implicated in the promotion of tumor progression, immune escape, epithelial-mesenchymal transition, and recruitment of myeloid-derived suppressor cells. Studies have demonstrated that high serum IL-8 levels correlate with poor prognosis in many malignant tumors. Preclinical studies have shown that IL-8 blockade may reduce mesenchymal features in tumor cells, making them less resistant to treatment.
11) C—C motif chemokine ligand 2 (CCL2) has been implicated in the recruitment of monocytes into the arterial wall during the disease process of atherosclerosis.
12) Macrophage Migration Inhibitory Factor (MIF) is a mediator of tumor progression; systemic inflammation; atherosclerosis; rheumatoid arthritis; and systemic lupus erythematosus, among others.

### Growth Factors

1) Fibroblast Growth Factor 1 (FGF1) can induce angiogenesis. FGF1 has been implicated in oncogenesis, cancer cell proliferation, resistance to anticancer therapies, and neoangiogenesis.
2) Fibroblast Growth Factor 2 (FGF2) has been implicated in oncogenesis, cancer cell proliferation, resistance to anticancer therapies, and neoangiogenesis.
3) Vascular Epithelial Growth Factor (VEGF-A) has been implicated in the vascularization and angiogenesis of tumors.
4) Transforming Growth Factor-β1 (TGF-β1) expression in the tumor microenvironment has been associated with a poor prognosis, and is implicated in TGF-β1 mediated tumor suppression via T-cell exclusion. TGF-β1 expression has also been implicated in hematological malignancies and fibrosis.
5) Transforming Growth Factor-β2 (TGF-β2) expression in the tumor microenvironment has been associated with a poor prognosis, and is implicated in TGF-β2 mediated tumor suppression via T-cell exclusion. TGF-β2 expression has also been implicated in hematological malignancies and fibrosis.
6) Placental Growth Factor (PGF) promotes cell tumor growth, and has been implicated in age-related macular degeneration (AMD) and choroidal neovascularization (CNV).

### Esterase

1)Cholinesterase has been implicated in cognitive disorders such as dementia and Alzheimer's disease.

### Coagulation Factors

1)Carboxypeptidase B2 has been implicated and targeted to inhibit thrombosis.
2)Coagulation Factor Xa is a mediator in the development of deep vein thrombosis and acute pulmonary embolism, and the risk of stroke and embolism in people with nonvalvular atrial fibrillation.
3)Coagulation Factor XI is a mediator in the development of deep vein thrombosis and acute pulmonary embolism, and the risk of stroke and embolism in people with nonvalvular atrial fibrillation.
4)Coagulation Factor XII has been implicated in the development of deep vein thrombosis and acute pulmonary embolism, and the risk of stroke and embolism in people with nonvalvular atrial fibrillation.
5)Coagulation Factor XIII has been implicated in the development of deep vein thrombosis and acute pulmonary embolism, and the risk of stroke and embolism in people with nonvalvular atrial fibrillation.
6) Prothrombin is involved in blood clot formation and arterial and venous thrombosis, and thromboembolism associated with atrial fibrillation.
7)Coagulation Factor VII is involved in blood clot formation and arterial and venous thrombosis, and thromboembolism associated with atrial fibrillation.
8)Coagulation Factor IX is involved in blood clot formation and arterial and venous thrombosis, and thromboembolism associated with atrial fibrillation.

### Extracellular Matrix Proteins

1)Neutrophil Elastase-Neutrophil elastase has been implicated in a number of disorders, including lung disease, chronic obstructive pulmonary disease, pneumonia, respiratory distress, and acute lung injury (ALI), and cystic fibrosis, as well as chronic kidney disease.
2) Fibronectin-1-Interfering with FN polymerization may attenuate myofibroblasts and fibrosis and improve cardiac function after ischemia/reperfusion (I/R) injury.
3) Thrombospondin-1-TSP-1 has been implicated in a number of diseases, including in promoting certain cancers such as breast cancer, prostate cancer, melanoma, SCLC, osteosarcoma, cutaneous squamous cell carcinoma, oral squamous cell carcinoma, papillary thyroid carcinoma, thyroid cancer, medulloblastoma, and fibrotic disorders such as diabetes, liver fibrosis, and in multiple myeloma.
4) Urokinase-type Plasminogen Activator (UPA)-UPA has been implicated in vascular diseases and cancer progression. Elevated expression levels of urokinase and several other components of the plasminogen activation system are found to be correlated with tumor malignancy.
5) Plasminogen Activator, Tissue Type (TPA)-PLA has been shown activated in various cancers including oral malignancy.
6) Plasminogen (PLG)-PLG has been implicated in tumor invasion and inflammation.
7) Plasminogen Activator Inhibitor-1 (PAI-1)-PAI-1 has been implicated in angiogenesis, metastasis, and poor prognosis in tumors, including, but not limited to, oral cancers and breast cancers.

### Peptidase

1) Kallikrein-1-Kallikrein has been implicated in adverse reactions in hereditary angioedema (HAE).
2) Plasma Kallikrein-Plasma kallikrein has been implicated in retinal dysfunction, the development of diabetic macular edema and hereditary angioedema (HAE).
3) Matrix Metallopeptidase-1-MMP-1 has been implicated in cardiovascular disease, development of fibrosis, and growth of certain cancers such as bladder cancer.
4) Phospholipase A2, Group IIA (PA2GA)-PA2GA has been implicated in a number of diseases, including cardiovascular diseases, atherosclerosis, immune disorders, and cancer.

### Lipase

1) Lipoprotein Lipase-Lipoprotein lipase has been implicated in the development of cardiovascular disease and obesity.
2) Phospholipase A2, Group IB (PA21B)-PA21B has been implicated in a number of diseases, including cardiovascular diseases, atherosclerosis, immune disorders and cancer.

### Convertase

1) Proprotein Convertase Subtilisin/Kexin Type 9 (PCSK-9)—PCSK-9 has been implicated in high blood cholesterol and the development of cardiovascular disease.

Certain extracellular protein targets include but are not limited to: SAA (serum amyloid A), amyloid light chains, antibodies to Klebsiella dipeptidase protein; Ig antibodies to anionic phospholipids and beta-2-glycoprotein-I; IL-13; MIF; Transthyretin (misfolded), IgG autoantibodies to thyroid peroxidase, thyroglobulin and TSH receptors; TNF-α; Protein arginine deiminase (PAD, PAD4); antibodies to citrullinated protein antibody (ACPA); anti-DNA antibodies; IL-17; Lysyl Oxidase 2 (LOXL2); IL-18; Blys; B cell activating factor (BAFF); CD40 (soluble); CXCL12; soluble PSMA; matrix metalloproteinase IX (MMP-9); hormone-sensitive lipase; lipoprotein- associated phospholipase A2; Factor Xa; DPP4; thrombin; PCSK9; ApoB-100; Complement component C3b; PKK (pre-kallikrein); Factor XI; PF⁴; Anti-vWF antibodies; anticardiolipin antibodies and lupus anticoagulant; FGF23 (fibroblast growth factor 23); Plasminogen activator inhibitor type 1 (PAI-1); Myeloperoxidase (MPO) extracellular; Myostatin; Beta2-m; suPAR (soluble urokinase plasminogen activator receptor); anti-ganglioside IgG; amyloid beta; Tau; CJD-associate prion; anti-ganglioside IgG; HTT; anti-ganglioside IgG; synuclein; elastase; PABA (protective antigen of Bacillus anthracis); edema factor; Botulinum toxin; C. difficile toxin B; hemolysin; tetanus toxin; IL-2; growth hormone and ACTH.

### VI. Exemplary Methods of Treatment of Diseases Mediated by Extracellular Proteins

The present invention can be used to treat any disorder that is mediated by the selected target disease-mediating extracellular protein. Nonlimiting examples of indications include autoimmune, other immune dysfunctions, complement mediated disorders, abnormal cellular proliferation, cancer, tumors, hematology-related disorders, renal disorders and liver disorders.

In certain embodiments, the degrader or its salt or composition as described herein is used in the treatment of an autoimmune disorder. In some aspects, the extracellular protein is an Ig, such as IgA or IgG. IgG degradation can treat for example, thyroid eye disease, myasthenia gravis, chronic inflammatory demyelinating polyneuropathy, and warm autoimmune hemolytic anemia.

Non-limiting examples of autoimmune disorders include: lupus, allograft rejection, autoimmune thyroid diseases (such as Graves' disease and Hashimoto's thyroiditis), autoimmune uveoretinitis, giant cell arteritis, inflammatory bowel diseases (including Crohn's disease, ulcerative colitis, regional enteritis, granulomatous enteritis, distal ileitis, regional ileitis, and terminal ileitis), diabetes, multiple sclerosis, pernicious anemia, psoriasis, rheumatoid arthritis, sarcoidosis, and scleroderma.

In an embodiment, the degrader or its salt or composition as described herein is used in the treatment of lupus. Non-limiting examples of lupus include lupus erythematosus, cutaneous lupus, discoid lupus erythematosus, chilblain lupus erythematosus, or lupus erythematosus-lichen planus overlap syndrome. Lupus erythematosus is a general category of disease that includes both systemic and cutaneous disorders. The systemic form of the disease can have cutaneous as well as systemic manifestations. However, there are also forms of the disease that are only cutaneous without systemic involvement. For example, SLE is an inflammatory disorder of unknown etiology that occurs predominantly in women, and is characterized by articular symptoms, butterfly erythema, recurrent pleurisy, pericarditis, generalized adenopathy, splenomegaly, as well as CNS involvement and progressive renal failure. The sera of most patients (over 98%) contain antinuclear antibodies, including anti-DNA antibodies. High titers of anti-DNA antibodies are essentially specific for SLE. Conventional treatment for this disease has been the administration of corticosteroids or immunosuppressants.

There are three forms of cutaneous lupus: chronic cutaneous lupus (also known as discoid lupus erythematosus or DLE), subacute cutaneous lupus, and acute cutaneous lupus. DLE is a disfiguring chronic disorder primarily affecting the skin with sharply circumscribed macules and plaques that display erythema, follicular plugging, scales, telangiectasia and atrophy. The condition is often precipitated by sun exposure, and the early lesions are erythematous, round scaling papules that are 5 to 10 mm in diameter and display follicular plugging. DLE lesions appear most commonly on the cheeks, nose, scalp, and ears, but they may also be generalized over the upper portion of the trunk, extensor surfaces of the extremities, and on the mucous membranes of the mouth. If left untreated, the central lesion atrophies and leaves a scar. Unlike SLE, antibodies against double-stranded DNA (e.g., DNA-binding test) are almost invariably absent in DLE.

Multiple Sclerosis is an autoimmune demyelinating disorder that is believed to be T lymphocyte dependent. MS generally exhibits a relapsing-remitting course or a chronic progressive course. The etiology of MS is unknown, however, viral infections, genetic predisposition, environment, and autoimmunity all appear to contribute to the disorder. Lesions in MS patients contain infiltrates of predominantly T lymphocyte mediated microglial cells and infiltrating macrophages. CD4+T lymphocytes are the predominant cell type present at these lesions. The hallmark of the MS lesion is plaque, an area of demyelination sharply demarcated from the usual white matter seen in MRI scans. Histological appearance of MS plaques varies with different stages of the disease. In active lesions, the blood-brain barrier is damaged, thereby permitting extravasation of serum proteins into extracellular spaces. Inflammatory cells can be seen in perivascular cuffs and throughout white matter. CD4+ T-cells, especially Th1, accumulate around postcapillary venules at the edge of the plaque and are also scattered in the white matter. In active lesions, up-regulation of adhesion molecules and markers of lymphocyte and monocyte activation, such as IL2-R and CD26 have also been observed. Demyelination in active lesions is not accompanied by destruction of oligodendrocytes. In contrast, during chronic phases of the disease, lesions are characterized by a loss of oligodendrocytes and hence, the presence of myelin oligodendrocyte glycoprotein (MOG) antibodies in the blood.

Diabetes can refer to either type 1 or type 2 diabetes. In some embodiments the degrader or its salt or composition as described herein is provided at an effective dose to treat a patient with type 1 diabetes. In one aspect the degrader or its salt or composition as described herein is provided at an effective dose to treat a patient with type 2 diabetes.

Type 1 diabetes is an autoimmune disease. An autoimmune disease results when the body's system for fighting infection (the immune system) turns against a part of the body. The pancreas then produces little or no insulin.

As examples, the degrader or its salt or composition as described herein is useful for treating or preventing a disorder selected from autoimmune oophoritis, endometriosis, autoimmune orchitis, Ord's thyroiditis, autoimmune enteropathy, coeliac disease, Hashimoto's encephalopathy, antiphospholipid syndrome (APLS) (Hughes syndrome), aplastic anemia, autoimmune lymphoproliferative syndrome (Canale-Smith syndrome), autoimmune neutropenia, Evans syndrome, pernicious anemia, pure red cell aplasia, thrombocytopenia, adipose dolorosa (Dercum's disease), adult onset Still's disease, ankylosing spondylitis, CREST syndrome, drug-induced lupus, eosinophilic fasciitis (Shulman's syndrome), Felty syndrome, IgG4-related disease, mixed connective tissue disease (MCTD), palindromic rheumatism (Hench-Rosenberg syndrome), Parry-Romberg syndrome, Parsonage-Turner syndrome, relapsing polychondritis (Meyenburg-Altherr-Uehlinger syndrome), retroperitonial fibrosis, rheumatic fever, Schnitzler syndrome, fibromyalgia, neuromyotonia (Isaac's disease), paraneoplastic degeneration, autoimmune inner ear disease, Meniere's disease, interstitial cystitis, autoimmune pancreatitis, zika virus-related disorders, chikungunya virus-related disorders, subacute bacterial endocarditis (SBE), IgA nephropathy, IgA vasculitis, polymyalgia rheumatic, rheumatoid vasculitis, alopecia areata, autoimmune progesterone dermatitis, dermatitis herpetiformis, erythema nodosum, gestational pemphigoid, hidradenitis suppurativa, lichen sclerosus, linear IgA disease (LAD), morphea, myositis, pityriasis lichenoides et varioliformis acuta, vitiligo post-myocardial infarction syndrome (Dressler's syndrome), post-pericardiotomy syndrome, autoimmune retinopathy, Cogan syndrome, Graves opthalmopathy, ligneous conjunctivitis, Mooren's ulcer, opsoclonus myoclonus syndrome, optic neuritis, retinocochleocerebral vasculopathy (Susac's syndrome), sympathetic opthalmia, Tolosa-Hunt syndrome, interstitial lung disease, antisynthetase syndrome, Addison's disease, autoimmune polyendocrine syndrome (APS) type I, autoimmune polyendocrine syndrome (APS) type II, autoimmune polyendocrine syndrome (APS) type III, disseminated sclerosis (multiple sclerosis, pattern II), rapidly progressing glomerulonephritis (RPGN), juvenile rheumatoid arthritis, enthesitis-related arthritis, reactive arthritis (Reiter's syndrome), autoimmune hepatitis or lupoid hepatitis, primary biliary cirrhosis (PBS), primary sclerosing cholangitis, microscopic colitis, latent lupus (undifferentiated connective tissue disease (UCTD)), acute disseminated encephalomyelitis (ADEM), acute motor axonal neuropathy, anti-n-methyl-D-aspartate receptor encephalitis, Balo concentric sclerosis (Schilders disease), Bickerstaff's encephalitis, chronic inflammatory demyelinating polyneuropathy, idiopathic inflammatory demyelinating disease, Lambert-Eaton mysathenic syndrome, Oshtoran syndrome, pediatric autoimmune neuropsychiatric disorder associated with streptococcus (PANDAS), progressive inflammatory neuropathy, restless leg syndrome, stiff person syndrome, Sydenhem syndrome, transverse myelitis, lupus vasculitis, leukocytoclastic vasculitis, Microscopic Polyangiitis, polymyositis or ischemic-reperfusion injury of the eye.

In certain aspects, an effective amount of the degrader or its salt or composition as described herein is used to treat a medical disorder mediated by the Targeted Extracellular Protein. For example, when the Targeted Extracellular Protein is a complement protein, for example complement factor B, factor D, factor H, C1s, C3, or C5, then the medical disorder to be treated may be an inflammatory or immune condition, a disorder mediated by the complement cascade (including a dysfunctional cascade), or alternative complement pathway-related disorder, a disorder or abnormality of a cell that adversely affects the ability of the cell to engage in or respond to normal complement activity, or an undesired complement-mediated response to a medical treatment, such as surgery or other medical procedure or a pharmaceutical or biopharmaceutical drug administration, a blood transfusion, or other allogenic tissue or fluid administration.

In some aspects, the disorder treated by the degrader or its salt or composition as described herein is selected from fatty liver and conditions stemming from fatty liver, such as nonalcoholic steatohepatitis (NASH), liver inflammation, cirrhosis and liver failure.

In other embodiments, the degrader or its salt or composition as described herein is used to modulate an immune response prior to or during surgery or other medical procedure. Non-limiting examples are the use in connection with acute or chronic graft versus host disease, which is a common complication as a result of allogeneic tissue transplant, and can also occur as a result of a blood transfusion.

In certain embodiments, the present invention provides a method of treating or preventing dermatomyositis by administering to a subject in need thereof an effective amount of the degrader or its salt or composition as described herein.

In certain embodiments, the present invention provides a method of treating or preventing amyotrophic lateral sclerosis by administering to a subject in need thereof an effective amount of the degrader or its salt or composition as described herein.

In certain embodiments, the present invention provides a method of treating or preventing abdominal aortic aneurysm, hemodialysis complications, hemolytic anemia, or hemodialysis by administering to a subject in need thereof an effective amount of the degrader or its salt or composition as described herein.

In certain embodiments, a method is provided for the treatment or prevention of cytokine or inflammatory reactions in response to the administration of pharmaceutical or biotherapeutic (e.g. CAR T-cell therapy or monoclonal antibody therapy) in a host by administering an effective amount of the degrader or its salt or composition as described herein. Various types of cytokine or inflammatory reactions may occur in response to a number of factors, such as the administrations of biotherapeutics. In one aspect, the cytokine or inflammatory reaction is cytokine release syndrome. In certain embodiments, the cytokine or inflammatory reaction is tumor lysis syndrome (which also leads to cytokine release). Symptoms of cytokine release syndrome range from fever, headache, and skin rashes to bronchospasm, hypotension and even cardiac arrest. Severe cytokine release syndrome is described as cytokine storm, and can be fatal.

In another embodiment, the disorder is episcleritis, idiopathic episcleritis, anterior episcleritis, or posterior episcleritis. In certain embodiments, the disorder is idiopathic anterior uveitis, HLA-B27 related uveitis, herpetic keratouveitis, Posner Schlossman syndrome, Fuch's heterochromic iridocyclitis, or cytomegalovirus anterior uveitis.

In another embodiment, the present invention provides a method of treating or preventing a C3 glomurenopathy by administering to a subject in need thereof an effective amount of the degrader or its salt or composition as described herein. In certain embodiments, the disorder is selected from dense deposit disease (DDD) and C3 glomerulonephritis (C3GN).

In yet another embodiment, the present invention provides a method of treating or preventing a IC-MPGN by administering to a subject in need thereof an effective amount of the degrader or its salt or composition as described herein.

In a further embodiment, the present invention provides a method of treating or preventing a paroxysmal nocturnal hemoglobinuria (PNH) by administering to a subject in need thereof an effective amount of the degrader or its salt or composition as described herein.

In another embodiment, the present invention provides a method of treating or preventing age-related macular degeneration (AMD) by administering to a subject in need thereof an effective amount of the degrader or its salt or composition as described herein.

In certain embodiments, the present invention provides a method of treating or preventing rheumatoid arthritis by administering to a subject in need thereof an effective amount of the degrader or its salt or composition as described herein.

In certain embodiments, the present invention provides a method of treating or preventing multiple sclerosis by administering to a subject in need thereof an effective amount of the degrader or its salt or composition as described herein.

In certain embodiments, the present invention provides a method of treating or preventing myasthenia gravis by administering to a subject in need thereof an effective amount of the degrader or its salt or composition as described herein.

In certain embodiments, the present invention provides a method of treating or preventing atypical hemolytic uremic syndrome (aHUS) by administering to a subject in need thereof an effective amount of the degrader or its salt or composition as described herein.

In certain embodiments, the present invention provides a method of treating or preventing neuromyelitis optica (NMO) by administering to a subject in need thereof an effective amount of the degrader or its salt or composition as described herein.

In yet other embodiments, the present invention provides a method of treating or preventing a disorder as described below by administering to a subject in need thereof an effective amount of the degrader or its salt or composition as described herein, including: for example: vitritis, sarcoidosis, syphilis, tuberculosis, or Lyme disease; retinal vasculitis, Eales disease, tuberculosis, syphilis, or toxoplasmosis; neuroretinitis, viral retinitis, or acute retinal necrosis; varicella zoster virus, herpes simplex virus, cytomegalovirus, Epstein-Barr virus, lichen planus, or Dengue-associated disease (e.g., hemorraghic Dengue Fever); Masquerade syndrome, contact dermatitis, trauma induced inflammation, UVB induced inflammation, eczema, granuloma annulare, or acne.

In additional embodiments, the disorder is selected from: acute myocardial infarction, aneurysm, cardiopulmonary bypass, dilated cardiomyopathy, complement activation during cardiopulmonary bypass operations, coronary artery disease, restenosis following stent placement, or percutaneous transluminal coronary angioplasty (PTCA); antibody-mediated transplant rejection, anaphylactic shock, anaphylaxis, allogenic transplant, humoral and vascular transplant rejection, graft dysfunction, graft-versus-host disease, Graves' disease, adverse drug reactions, or chronic graft vasculopathy; allergic bronchopulmonary aspergillosis, allergic neuritis, drug allergy, radiation-induced lung injury, eosinophilic pneumonia, radiographic contrast media allergy, bronchiolitis obliterans, or interstitial pneumonia; parkinsonism-dementia complex, sporadic frontotemporal dementia, frontotemporal dementia with Parkinsonism linked to chromosome 17, frontotemporal lobar degeneration, tangle only dementia, cerebral amyloid angiopathy, cerebrovascular disorder, certain forms of frontotemporal dementia, chronic traumatic encephalopathy (CTE), PD with dementia (PDD), argyrophilic grain dementia, dementia pugilistica, dementia with Lewy Bodies (DLB), or multi-infarct dementia; Creutzfeldt-Jakob disease, Huntington's disease, multifocal motor neuropathy (MMN), prion protein cerebral amyloid angiopathy, polymyositis, postencephalitic parkinsonism, subacute sclerosing panencephalitis, non-Guamanian motor neuron disease with neurofibrillary tangles, neural regeneration, or diffuse neurofibrillary tangles with calcification.

In certain embodiments the disorder is Graves' eye disease.

In certain embodiments the disorder is Graves' ophthalmopathy.

In certain embodiments the disorder is Graves' orbitopathy.

In certain embodiments the disorder is thyroid eye disease.

In certain embodiments the disorder is neuromyelitis optica spectrum disorder.

In certain embodiments the disorder is myelin oligodendrocyte glycoprotein antibody-associated disease (MOGAD).

In further embodiments, the disorder is selected from: atopic dermatitis, dermatitis, dermatomyositis bullous pemphigoid, scleroderma, sclerodermatomyositis, psoriatic arthritis, pemphigus vulgaris, Discoid lupus erythematosus, cutaneous lupus, chilblain lupus erythematosus, or lupus erythematosus-lichen planus overlap syndrome; cryoglobulinemic vasculitis, mesenteric/enteric vascular disorder, peripheral vascular disorder, antineutrophil cytoplasm antibody (ANCA)- associated vasculitis (AAV), IL-2 induced vascular leakage syndrome, or immune complex vasculitis;angioedema, low platelets (HELLP) syndrome, sickle cell disease, platelet refractoriness, red cell casts, or typical or infectious hemolytic uremic syndrome (tHUS); hematuria, hemorrhagic shock, drug-induced thrombocytopenia, autoimmune hemolytic anemia (AIHA), azotemia, blood vessel and/or lymph vessel inflammation, rotational atherectomy, or delayed hemolytic transfusion reaction; British type amyloid angiopathy, Buerger's disease, bullous pemphigoid, C1q nephropathy, cancer, or catastrophic antiphospholipid syndrome.

In other embodiments, the disorder is selected from: wet (exudative) AMD, dry (non-exudative) AMD, chorioretinal degeneration, choroidal neovascularization (CNV), choroiditis, loss of RPE function, loss of vision (including loss of visual acuity or visual field), loss of vision from AMD, retinal damage in response to light exposure, retinal degeneration, retinal detachment, retinal dysfunction, retinal neovascularization (RNV), retinopathy of prematurity, pathological myopia, or RPE degeneration; pseudophakic bullous keratopathy, symptomatic macular degeneration related disorder, optic nerve degeneration, photoreceptor degeneration, cone degeneration, loss of photoreceptor cells, pars planitis, scleritis, proliferative vitreoretinopathy, or formation of ocular drusen; chronic urticaria, Churg-Strauss syndrome, cold agglutinin disease (CAD), corticobasal degeneration (CBD), cryoglobulinemia, cyclitis, damage of the Bruch's membrane, Degos disease, diabetic angiopathy, elevated liver enzymes, endotoxemia, epidermolysis bullosa, or epidermolysis bullosa acquisita; essential mixed cryoglobulinemia, excessive blood urea nitrogen-BUN, focal segmental glomerulosclerosis, Gerstmann-Straussler-Scheinker disease, giant cell arteritis, gout, Hallervorden-Spatz disease, Hashimoto's thyroiditis; Henoch-Schonlein purpura nephritis, or abnormal urinary sediments; hepatitis, hepatitis A, hepatitis B, hepatitis C or human immunodeficiency virus (HIV), a viral infection more generally, for example selected from Flaviviridae, Retroviruses, Coronaviridae, Poxviridae, Adenoviridae, Herpesviridae, Caliciviridae, Reoviridae, Picornaviridae, Togaviridae, Orthomyxoviridae, Rhabdoviridae, or Hepadnaviridae; Neisseria meningitidis, shiga toxin E. coli-related hemolytic uremic syndrome (STEC-HUS), hemolytic uremic syndrome (HUS); Streptococcus, or poststreptococcal glomerulonephritis.

In further embodiments, the disorder is selected from: hyperlipidemia, hypertension, hypoalbuminemia, hypobolemic shock, hypocomplementemic urticarial vasculitis syndrome, hypophosphastasis, hypovolemic shock, idiopathic pneumonia syndrome, or idiopathic pulmonary fibrosis; inclusion body myositis, intestinal ischemia, iridocyclitis, iritis, juvenile chronic arthritis, Kawasaki's disease (arteritis), or lipiduria; membranoproliferative glomerulonephritis (MPGN) I, microscopic polyangiitis, mixed cryoglobulinemia, molybdenum cofactor deficiency (MoCD) type A, pancreatitis, panniculitis, Pick's disease, polyarteritis nodosa (PAN), progressive subcortical gliosis, proteinuria, reduced glomerular filtration rate (GFR), or renovascular disorder; multiple organ failure, multiple system atrophy (MSA), myotonic dystrophy, Niemann-Pick disease type C, chronic demyelinating diseases, or progressive supranuclear palsy; spinal cord injury, spinal muscular atrophy, spondyloarthropathies, Reiter's syndrome, spontaneous fetal loss, recurrent fetal loss, pre-eclampsia, synucleinopathy, Takayasu's arteritis, post-partum thryoiditis, thyroiditis, Type I cryoglobulinemia, Type II mixed cryoglobulinemia, Type III mixed cryoglobulinemia, ulcerative colitis, uremia, urticaria, venous gas embolus (VGE), or Wegener's granulomatosis; von Hippel-Lindau disease, histoplasmosis of the eye, hard drusen, soft drusen, pigment clumping, or photoreceptor and/or retinal pigmented epithelia (RPE) loss.

Examples of eye disorders that may be treated according to the compositions and methods disclosed herein include amoebic keratitis, fungal keratitis, bacterial keratitis, viral keratitis, onchorcercal keratitis, bacterial keratoconjunctivitis, viral keratoconjunctivitis, corneal dystrophic diseases, Fuchs' endothelial dystrophy, Sjogren's syndrome, Stevens-Johnson syndrome, autoimmune dry eye diseases, environmental dry eye diseases, corneal neovascularization diseases, post-corneal transplant rejection prophylaxis and treatment, autoimmune uveitis, infectious uveitis, posterior uveitis (including toxoplasmosis), pan-uveitis, an inflammatory disease of the vitreous or retina, endophthalmitis prophylaxis and treatment, macular edema, macular degeneration, age related macular degeneration, proliferative and non-proliferative diabetic retinopathy, hypertensive retinopathy, an autoimmune disease of the retina, primary and metastatic intraocular melanoma, other intraocular metastatic tumors, open angle glaucoma, closed angle glaucoma, pigmentary glaucoma and combinations thereof.

In other embodiments, the disorder is selected from glaucoma, diabetic retinopathy, blistering cutaneous diseases (including bullous pemphigoid, pemphigus, and epidermolysis bullosa), ocular cicatrical pemphigoid, uveitis, adult macular degeneration, diabetic retinopa retinitis pigmentosa, macular edema, diabetic macular edema, Behcet's uveitis, multifocal choroiditis, Vogt-Koyangi-Harada syndrome, imtermediate uveitis, birdshot retino-chorioditis, sympathetic ophthalmia, ocular dicatricial pemphigoid, ocular pemphigus, nonartertic ischemic optic neuropathy, postoperative inflammation, and retinal vein occlusion, or central retinal vein occulusion (CVRO).

Disorders that may be treated or prevented by the degrader or its salt or composition as described herein also include, but are not limited to: hereditary angioedema, capillary leak syndrome, hemolytic uremic syndrome (HUS), neurological disorders, Guillain Barre Syndrome, diseases of the central nervous system and other neurodegenerative conditions, glomerulonephritis (including membrane proliferative glomerulonephritis), SLE nephritis, proliferative nephritis, liver fibrosis, tissue regeneration and neural regeneration, or Barraquer-Simons Syndrome; inflammatory effects of sepsis, systemic inflammatory response syndrome (SIRS), disorders of inappropriate or undesirable complement activation, interleukin-2 induced toxicity during IL-2 therapy, inflammatory disorders, inflammation of autoimmune diseases, system lupus erythematosus (SLE), lupus nephritides, arthritis, immune complex disorders and autoimmune diseases, systemic lupus, or lupus erythematosus; ischemia/reperfusion injury (I/R injury), myocardial infarction, myocarditis, post-ischemic reperfusion conditions, balloon angioplasty, atherosclerosis, post-pump syndrome in cardiopulmonary bypass or renal bypass, renal ischemia, mesenteric artery reperfusion after aortic reconstruction, antiphospholipid syndrome, autoimmune heart disease, ischemia-reperfusion injuries, obesity, or diabetes; Alzheimer's dementia, stroke, schizophrenia, traumatic brain injury, trauma, Parkinson's disease, epilepsy, transplant rejection, prevention of fetal loss, biomaterial reactions (e.g. in hemodialysis, inplants), hyperacute allograft rejection, xenograft rejection, transplantation, psoriasis, burn injury, thermal injury including burns or frostbite, or crush injury; asthma, allergy, acute respiratory distress syndrome (ARDS), cystic fibrosis, adult respiratory distress syndrome, dyspnea, hemoptysis, chronic obstructive pulmonary disease (COPD), emphysema, pulmonary embolisms and infarcts, pneumonia, fibrogenic dust diseases, inert dusts and minerals (e.g., silicon, coal dust, beryllium, and asbestos), pulmonary fibrosis, organic dust diseases, chemical injury (due to irritant gases and chemicals, e.g., chlorine, phosgene, sulfur dioxide, hydrogen sulfide, nitrogen dioxide, ammonia, and hydrochloric acid), smoke injury, thermal injury (e.g., burn, freeze), bronchoconstriction, hypersensitivity pneumonitis, parasitic diseases, Goodpasture's Syndrome (anti-glomerular basement membrane nephritis), pulmonary vasculitis, Pauci-immune vasculitis, or immune complex- associated inflammation.

In another embodiment, a method for the treatment of sickle cell in a host is provided that includes the administration of an effective amount of the degrader or its salt or composition as described herein. In certain embodiments, a method for the treatment of immunothrombocytopenia purpura (ITP), thrombotic thrombocytopeniarpura (TTP), or idiopathic thrombocytopenia purpura (ITP) in a host is provided that includes the administration of an effective amount of the degrader or its salt or composition as described herein. In certain embodiments, a method for the treatment of ANCA-vasculitis in a host is provided that includes the administration of an effective amount of the degrader or its salt or composition as described herein. In certain embodiments, a method for the treatment of IgA nephropathy in a host is provided that includes the administration of an effective amount of the degrader or its salt or composition as described herein. In certain embodiments, a method for the treatment of rapidly progressing glomerulonephritis (RPGN), in a host is provided that includes the administration of an effective amount of the degrader or its salt or composition as described herein. In certain embodiments, a method for the treatment of lupus nephritis, in a host is provided that includes the administration of an effective amount of the degrader or its salt or composition as described herein. In certain embodiments, a method for the treatment of hemorraghic dengue fever, in a host is provided that includes the administration of an effective amount of the degrader or its salt or composition as described herein.

In another aspect, an effective amount of the degrader or its salt or composition as described herein is used to treat an abnormal proliferation disorder such as a tumor or cancer.

Non-limiting examples of cancers that can be treated according to the present invention include, but are not limited to, acoustic neuroma, adenocarcinoma, adrenal gland cancer, anal cancer, angiosarcoma (e.g., lymphangiosarcoma, lymphangioendotheliosarcoma, hemangiosarcoma), appendix cancer, benign monoclonal gammopathy, biliary cancer (e.g., cholangiocarcinoma), bladder cancer, breast cancer (e.g., adenocarcinoma of the breast, papillary carcinoma of the breast, mammary cancer, medullary carcinoma of the breast), brain cancer (e.g., meningioma; glioma, e.g., astrocytoma, oligodendroglioma; medulloblastoma), bronchus cancer, carcinoid tumor, cervical cancer (e.g., cervical adenocarcinoma), choriocarcinoma, chordoma, craniopharyngioma, colorectal cancer (e.g., colon cancer, rectal cancer, colorectal adenocarcinoma), epithelial carcinoma, ependymoma, endotheliosarcoma (e.g., Kaposi's sarcoma, multiple idiopathic hemorrhagic sarcoma), endometrial cancer (e.g., uterine cancer, uterine sarcoma), esophageal cancer (e.g., adenocarcinoma of the esophagus, Barrett's adenocarinoma), Ewing's sarcoma, eye cancer (e.g., intraocular melanoma, retinoblastoma), familiar hypereosinophilia, gall bladder cancer, gastric cancer (e.g., stomach adenocarcinoma), gastrointestinal stromal tumor (GIST), head and neck cancer (e.g., head and neck squamous cell carcinoma, oral cancer (e.g., oral squamous cell carcinoma (OSCC), throat cancer (e.g., laryngeal cancer, pharyngeal cancer, nasopharyngeal cancer, oropharyngeal cancer)), hematopoietic cancers (e.g., leukemia such as acute lymphocytic leukemia (ALL)—also known as acute lymphoblastic leukemia or acute lymphoid leukemia (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma (DLBCL)), follicular lymphoma, chronic lymphocytic leukemia/small lymphocytic lymphoma (CLL/SLL), mantle cell lymphoma (MCL), marginal zone B-cell lymphomas (e.g., mucosa- associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., “Waldenström's macroglobulinemia”), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma); a mixture of one or more leukemia/lymphoma as described above; and multiple myeloma (MM)), heavy chain disease (e.g., alpha chain disease, gamma chain disease, mu chain disease), hemangioblastoma, inflammatory myofibroblastic tumors, immunocytic amyloidosis, kidney cancer (e.g., nephroblastoma a.k.a. Wilms' tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), adenocarcinoma of the lung), leiomyosarcoma (LMS), mastocytosis (e.g., systemic mastocytosis), myelodysplastic syndrome (MDS), mesothelioma, myeloproliferative disorder (MPD) (e.g., polycythemia Vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)), neuroblastoma, neurofibroma (e.g., neurofibromatosis (NF) type 1 or type 2, schwannomatosis), neuroendocrine cancer (e.g., gastroenteropancreatic neuroendoctrine tumor (GEP-NET), carcinoid tumor), osteosarcoma, ovarian cancer (e.g., cystadenocarcinoma, ovarian embryonal carcinoma, ovarian adenocarcinoma), papillary adenocarcinoma, pancreatic cancer (e.g., pancreatic andenocarcinoma, intraductal papillary mucinous neoplasm (IPMN), Islet cell tumors), penile cancer (e.g., Paget's disease of the penis and scrotum), pinealoma, primitive neuroectodermal tumor (PNT), prostate cancer (e.g., prostate adenocarcinoma), rectal cancer, rhabdomyosarcoma, salivary gland cancer, skin cancer (e.g., squamous cell carcinoma (SCC), keratoacanthoma (KA), melanoma, basal cell carcinoma (BCC)), small bowel cancer (e.g., appendix cancer), soft tissue sarcoma (e.g., malignant fibrous histiocytoma (MFH), liposarcoma, malignant peripheral nerve sheath tumor (MPNST), chondrosarcoma, fibrosarcoma, myxosarcoma), sebaceous gland carcinoma, sweat gland carcinoma, synovioma, testicular cancer (e.g., seminoma, testicular embryonal carcinoma), thyroid cancer (e.g., papillary carcinoma of the thyroid, papillary thyroid carcinoma (PTC), medullary thyroid cancer), urethral cancer, vaginal cancer and vulvar cancer (e.g., Paget's disease of the vulva).

In another embodiment, the disorder is myelodysplastic syndrome (MDS).

In certain embodiments, the cancer is a hematopoietic cancer. In certain embodiments, the hematopoietic cancer is a lymphoma. In certain embodiments, the hematopoietic cancer is a leukemia. In certain embodiments, the leukemia is acute myelocytic leukemia (AML).

In certain embodiments, the proliferative disorder is a myeloproliferative neoplasm. In certain embodiments, the myeloproliferative neoplasm (MPN) is primary myelofibrosis (PMF).

In certain embodiments, the cancer is a solid tumor. A solid tumor, as used herein, refers to an abnormal mass of tissue that usually does not contain cysts or liquid areas. Different types of solid tumors are named for the type of cells that form them. Examples of classes of solid tumors include, but are not limited to, sarcomas, carcinomas, and lymphomas, as described above herein. Additional examples of solid tumors include, but are not limited to, squamous cell carcinoma, colon cancer, breast cancer, prostate cancer, lung cancer, liver cancer, pancreatic cancer, and melanoma.

Abnormal cellular proliferation, notably hyperproliferation, can occur as a result of a wide variety of factors, including genetic mutation, infection, exposure to toxins, autoimmune disorders, and benign or malignant tumor induction.

There are a number of skin disorders associated with cellular hyperproliferation. Psoriasis, for example, is a benign disease of human skin generally characterized by plaques covered by thickened scales. The disease is caused by increased proliferation of epidermal cells of unknown cause. Chronic eczema is also associated with significant hyperproliferation of the epidermis. Other diseases caused by hyperproliferation of skin cells include atopic dermatitis, lichen planus, warts, pemphigus vulgaris, actinic keratosis, basal cell carcinoma and squamous cell carcinoma.

Other hyperproliferative cell disorders include blood vessel proliferation disorders, fibrotic disorders, autoimmune disorders, graft-versus-host rejection, tumors and cancers.

Blood vessel proliferative disorders include angiogenic and vasculogenic disorders. Proliferation of smooth muscle cells in the course of development of plaques in vascular tissue cause, for example, restenosis, retinopathies and atherosclerosis. Both cell migration and cell proliferation play a role in the formation of atherosclerotic lesions.

Fibrotic disorders are often due to the abnormal formation of an extracellular matrix. Examples of fibrotic disorders include hepatic cirrhosis and mesangial proliferative cell disorders. Hepatic cirrhosis is characterized by the increase in extracellular matrix constituents resulting in the formation of a hepatic scar. Hepatic cirrhosis can cause diseases such as cirrhosis of the liver. An increased extracellular matrix resulting in a hepatic scar can also be caused by viral infection such as hepatitis. Lipocytes appear to play a major role in hepatic cirrhosis.

Mesangial disorders are brought about by abnormal proliferation of mesangial cells. Mesangial hyperproliferative cell disorders include various human renal diseases, such as glomerulonephritis, diabetic nephropathy, malignant nephrosclerosis, thrombotic micro-angiopathy syndromes, transplant rejection, and glomerulopathies.

Another disease with a proliferative component is rheumatoid arthritis. Rheumatoid arthritis is generally considered an autoimmune disease that is thought to be associated with activity of autoreactive T cells, and to be caused by autoantibodies produced against collagen and IgE.

Other disorders that can include an abnormal cellular proliferative component include Bechet's syndrome, acute respiratory distress syndrome (ARDS), ischemic heart disease, post-dialysis syndrome, leukemia, acquired immune deficiency syndrome, vasculitis, lipid histiocytosis, septic shock and inflammation in general.

In certain embodiments, the condition is associated with an immune response.

Cutaneous contact hypersensitivity and asthma are just two examples of immune responses that can be associated with significant morbidity. Others include atopic dermatitis, eczema, Sjogren's Syndrome, including keratoconjunctivitis sicca secondary to Sjogren's Syndrome, alopecia areata, allergic responses due to arthropod bite reactions, Crohn's disease, aphthous ulcer, iritis, conjunctivitis, keratoconjunctivitis, ulcerative colitis, cutaneous lupus erythematosus, scleroderma, vaginitis, proctitis, and drug eruptions. These conditions may result in any one or more of the following symptoms or signs: itching, swelling, redness, blisters, crusting, ulceration, pain, scaling, cracking, hair loss, scarring, or oozing of fluid involving the skin, eye, or mucosal membranes.

In atopic dermatitis, and eczema in general, immunologically mediated leukocyte infiltration (particularly infiltration of mononuclear cells, lymphocytes, neutrophils, and eosinophils) into the skin importantly contributes to the pathogenesis of these diseases. Chronic eczema also is associated with significant hyperproliferation of the epidermis. Immunologically mediated leukocyte infiltration also occurs at sites other than the skin, such as in the airways in asthma and in the tear producing gland of the eye in keratoconjunctivitis sicca.

In other non-limiting embodiments, degraders of the present invention are used as topical agents in treating contact dermatitis, atopic dermatitis, eczematous dermatitis, psoriasis, Sjogren's Syndrome, including keratoconjunctivitis sicca secondary to Sjogren's Syndrome, alopecia areata, allergic responses due to arthropod bite reactions, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, and drug eruptions. The novel method may also be useful in reducing the infiltration of skin by malignant leukocytes in diseases such as mycosis fungoides. These compounds can also be used to treat an aqueous-deficient dry eye state (such as immune mediated keratoconjunctivitis) in a patient suffering therefrom, by administering the compound topically to the eye.

Exemplary cancers which may be treated by the present disclosed compounds either alone or in combination with at least one additional anti-cancer agent include squamous-cell carcinoma, basal cell carcinoma, adenocarcinoma, hepatocellular carcinomas, and renal cell carcinomas, cancer of the bladder, bowel, breast, cervix, colon, esophagus, head, kidney, liver, lung, neck, ovary, pancreas, prostate, and stomach; leukemias; benign and malignant lymphomas, particularly Burkitt's lymphoma and Non-Hodgkin's lymphoma; benign and malignant melanomas; myeloproliferative diseases; sarcomas, including Ewing's sarcoma, hemangiosarcoma, Kaposi's sarcoma, liposarcoma, myosarcomas, peripheral neuroepithelioma, synovial sarcoma, gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas; bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, melanoma; carcinosarcoma, Hodgkin's disease, Wilms' tumor and teratocarcinomas. Additional cancers which may be treated using the disclosed compounds according to the present invention include, for example, acute granulocytic leukemia, acute lymphocytic leukemia (ALL), acute myelogenous leukemia (AML), adenocarcinoma, adenosarcoma, adrenal cancer, adrenocortical carcinoma, anal cancer, anaplastic astrocytoma, angiosarcoma, appendix cancer, astrocytoma, Basal cell carcinoma, B-Cell lymphoma, bile duct cancer, bladder cancer, bone cancer, bone marrow cancer, bowel cancer, brain cancer, brain stem glioma, breast cancer, triple (estrogen, progesterone and HER-2) negative breast cancer, double negative breast cancer (two of estrogen, progesterone and HER-2 are negative), single negative (one of estrogen, progesterone and HER-2 is negative), estrogen-receptor positive, HER2-negative breast cancer, estrogen receptor-negative breast cancer, estrogen receptor positive breast cancer, metastatic breast cancer, luminal A breast cancer, luminal B breast cancer, Her2-negative breast cancer, HER2-positive or negative breast cancer, progesterone receptor-negative breast cancer, progesterone receptor-positive breast cancer, recurrent breast cancer, carcinoid tumors, cervical cancer, cholangiocarcinoma, chondrosarcoma, chronic lymphocytic leukemia (CLL), chronic myelogenous leukemia (CML), colon cancer, colorectal cancer, craniopharyngioma, cutaneous lymphoma, cutaneous melanoma, diffuse astrocytoma, ductal carcinoma in situ (DCIS), endometrial cancer, ependymoma, epithelioid sarcoma, esophageal cancer, ewing sarcoma, extrahepatic bile duct cancer, eye cancer, fallopian tube cancer, fibrosarcoma, gallbladder cancer, gastric cancer, gastrointestinal cancer, gastrointestinal carcinoid cancer, gastrointestinal stromal tumors (GIST), germ cell tumor glioblastoma multiforme (GBM), glioma, hairy cell leukemia, head and neck cancer, hemangioendothelioma, Hodgkin lymphoma, hypopharyngeal cancer, infiltrating ductal carcinoma (IDC), infiltrating lobular carcinoma (ILC), inflammatory breast cancer (IBC), intestinal Cancer, intrahepatic bile duct cancer, invasive/infiltrating breast cancer, Islet cell cancer, jaw cancer, Kaposi sarcoma, kidney cancer, laryngeal cancer, leiomyosarcoma, leptomeningeal metastases, leukemia, lip cancer, liposarcoma, liver cancer, lobular carcinoma in situ, low-grade astrocytoma, lung cancer, lymph node cancer, lymphoma, male breast cancer, medullary carcinoma, medulloblastoma, melanoma, meningioma, Merkel cell carcinoma, mesenchymal chondrosarcoma, mesenchymous, mesothelioma metastatic breast cancer, metastatic melanoma metastatic squamous neck cancer, mixed gliomas, monodermal teratoma, mouth cancer mucinous carcinoma, mucosal melanoma, multiple myeloma, Mycosis Fungoides, myelodysplastic syndrome, nasal cavity cancer, nasopharyngeal cancer, neck cancer, neuroblastoma, neuroendocrine tumors (NETs), non-Hodgkin's lymphoma, non-small cell lung cancer (NSCLC), oat cell cancer, ocular cancer, ocular melanoma, oligodendroglioma, oral cancer, oral cavity cancer, oropharyngeal cancer, osteogenic sarcoma, osteosarcoma, ovarian cancer, ovarian epithelial cancer ovarian germ cell tumor, ovarian primary peritoneal carcinoma, ovarian sex cord stromal tumor, Paget's disease, pancreatic cancer, papillary carcinoma, paranasal sinus cancer, parathyroid cancer, pelvic cancer, penile cancer, peripheral nerve cancer, peritoneal cancer, pharyngeal cancer, pheochromocytoma, pilocytic astrocytoma, pineal region tumor, pineoblastoma, pituitary gland cancer, primary central nervous system (CNS) lymphoma, prostate cancer, rectal cancer, renal cell carcinoma, renal pelvis cancer, rhabdomyosarcoma, salivary gland cancer, soft tissue sarcoma, bone sarcoma, sarcoma, sinus cancer, skin cancer, small cell lung cancer (SCLC), small intestine cancer, spinal cancer, spinal column cancer, spinal cord cancer, squamous cell carcinoma, stomach cancer, synovial sarcoma, T-cell lymphoma, testicular cancer, throat cancer, thymoma/thymic carcinoma, thyroid cancer, tongue cancer, tonsil cancer, transitional cell cancer, tubal cancer, tubular carcinoma, undiagnosed cancer, ureteral cancer, urethral cancer, uterine adenocarcinoma, uterine cancer, uterine sarcoma, vaginal cancer, vulvar cancer, T-cell lineage acute lymphoblastic leukemia (T-ALL), T-cell lineage lymphoblastic lymphoma (T-LL), peripheral T-cell lymphoma, Adult T-cell leukemia, Pre-B ALL, Pre-B lymphomas, large B-cell lymphoma, Burkitts lymphoma, B-cell ALL, Philadelphia chromosome positive ALL, Philadelphia chromosome positive CML, juvenile myelomonocytic leukemia (JMML), acute promyelocytic leukemia (a subtype of AML), large granular lymphocytic leukemia, Adult T-cell chronic leukemia, diffuse large B cell lymphoma, follicular lymphoma; Mucosa-Associated Lymphatic Tissue lymphoma (MALT), small cell lymphocytic lymphoma, mediastinal large B cell lymphoma, nodal marginal zone B cell lymphoma (NMZL); splenic marginal zone lymphoma (SMZL); intravascular large B-cell lymphoma; primary effusion lymphoma; or lymphomatoid granulomatosis; B-cell prolymphocytic leukemia; splenic lymphoma/leukemia, unclassifiable, splenic diffuse red pulp small B-cell lymphoma; lymphoplasmacytic lymphoma; heavy chain diseases, for example, Alpha heavy chain disease, Gamma heavy chain disease, Mu heavy chain disease, plasma cell myeloma, solitary plasmacytoma of bone; extraosseous plasmacytoma; primary cutaneous follicle center lymphoma, T cell/histocyte rich large B-cell lymphoma, DLBCL associated with chronic inflammation; Epstein-Barr virus (EBV)+DLBCL of the elderly; primary mediastinal (thymic) large B-cell lymphoma, primary cutaneous DLBCL, leg type, ALK+large B-cell lymphoma, plasmablastic lymphoma; large B-cell lymphoma arising in HHV8- associated multicentric, Castleman disease; B-cell lymphoma, unclassifiable, with features intermediate between diffuse large B-cell lymphoma, or B-cell lymphoma, unclassifiable, with features intermediate between diffuse large B-cell lymphoma and classical Hodgkin lymphoma.

Nonlimiting general examples of disorders mediated by extracellular proteins also include, but are not limited to: AMD, macular edema, DME, diabetic retinopathy, mCNV; neurodegenerative disorders, metastatic colorectal cancer, non-squamous non-small-cell lung carcinoma, GMB, metastatic renal cell carcinoma, cervical cancer, AA amyloidosis, amyloid light chain (AL) amyloidosis, ankylosing spondylitis, antiphospholipid Ab syndrome, asthma, progression of parasite Schistosoma mansoni infection (IL-13), ATTR amyloidosis, Behcet syndrome, sepsis, inflammation, rheumatoid arthritis, atherosclerosis, ischemia/reperfusion injury; MGUS, Necrobiotic xanthogranuloma, JIA, psoriatic arthritis, plaque psoriasis, Crohn's disease, ulcerative colitis, Hidradenitis suppurativa uveitis; GvH disease; Castleman's disease, liver fibrosis, Still's Disease; cutaneous skin diseases including atopic dermatitis, transplant rejection, multiple myeloma, osteosclerotic multiple myeloma with peripheral neuropathy; pancreatic tumors; paraproteinemia (NR), prostate, gastric cancer; glioblastoma multiforme; acute coronary syndrome; hyperlipidemia (Rare/Broad), chronic urticaria, scleroderma, scleromyxedema, hereditary angioedema, clotting disorders, heparin-induced thrombocytopenia; Acquired Von Willebrand disease (AVWD), antiphospholipid antibody syndrome (APS or APLS); cryoglobulinemia; granulomatosis with polyangiitis (Wegener's)-sub-type of ANCA- associated vasculitis; idiopathic (Immune); thrombocytopenia purpura; IgG4-RD; Non-IgM MGUS; X-linked hypophosphatemia; Multiple System Atrophy (MSA), Parkinson's disease, Cachexia, Sarcopenia, Sporadic inclusion body myositis, muscular dystrophy, COPD; rhabdomyolysis; dialysis-related amyloidosis; focal segmental glomerulosclerosis (FSGS); IgA nephropathy (IgAN) and Henoch Schönlein Purpura (HSP); acute disseminated encephalomyelitis (ADEM); acute inflammatory demyelinating polyneuropathy (AIDP); Guillaine-Barre Syndrome; Alzheimer′ disease & FTD; chronic inflammatory demyelinating polyneuropathy (CIDP); Creutzfeldt-Jakob disease (CJD); Huntington's disease; Miller Fisher Syndrome; Neuromyelitis optica spectrum disorder (NMOSD); Opsoclonus-myoclonus syndrome; PANDAS syndrome (pediatric autoimmune neuropsychiatric disorders associated with Streptcoccal infections); Transverse myelitis; Emphysema, respiratory failure; Anthrax; Botulism; Sepsis; Staph. aureus toxic shock syndrome; Tetanus; Transplantation; Acromegaly; Cushing's disease; prion disease; secondary membranous nephropathy; and vasculitis.

### VII. Methods of Manufacture and Examples

### Example 1. HTRF-Based Ternary Complex Inhibition Assay for Determining Relative Affinity of IGF-1IR Ligands

The apparent binding affinity of Insulin-like growth factor 2 receptor (IGF-1IR, also called the cation- independent mannose-6-phosphate receptor (CI-MPR)) ligands was determined indirectly by competing with a probe molecule for binding to IGF-1IR. The probe is a Mannose 6-Phosphate Ligand (“Ligand”) and binds to hIGF-IIR and IgG1 to form a hIGF-IIR: Ligand: IgG1 ternary complex. The ternary complex was detected by an HTRF readout. The ternary complex is disrupted in the presence of IGF-1IR ligand, resulting in decreased HTRF signal. IGF-1IR ligands of 3-fold dilution series were prepared from 10 mM DMSO stock on a 384-well LDV microplate using Echo Acoustic Liquid Handler (Beckman Coulter Life Sciences) followed by transfer of 600 nL of the solutions to an OptiPlate 384-well microplate (PerkinElmer, 6007290). Manose-6-phosphate (M6P; J&K, 530168) and glucose-6-phosphate (G6P; Sigma, V900924) were prepared from 100 mM stock in the same manner and were used as positive and negative controls, respectively. The wells used as 0% and 100% inhibition controls were prepared by adding 600 nL DMSO. 20 nL of 50 μM MTAC probe was transferred to all wells except the wells used as 100% Inhibition control. 9.4 μL working solution containing 65.1 nM of Biotinylated-IgG1 (Viva 20211108) and 65.1 nM of His-tagged hIGF-IIR (R&D Systems, 6418-GR) in assay buffer 50 mM Tris, pH 7.5, 150 mM NaCl, 50 mM CaCl₂, 0.01% Tween-20 was transferred to all wells. The plate was incubated at room temperature for 60 min with gentle shaking to allow molecular interaction to reach equilibration. Following this, 10 μL of HTRF detection solution containing 15 nM Streptavidin-d2 (PerkinElmer, 610SADLA) and 0.7 nM MAb Anti 6HIS—Tb cryptate (PerkinElmer, 61HISTLA) in PPI Terbium detection buffer (PerkinElmer, 61DB10RDF) was added. The assay plate was incubated at room temperature with gentle shaking for 60 min to allow HTRF signal to develop. The plate was gently spun on a centrifuge then placed on a EnVision plate reader (Perkin Elmer, EnVision 2104) to read fluorescence ratio of 665 nm/615 nm. Percent inhibitions were calculated with this equation: % I=100* [1−(X−100% I/(0% I−100% I)] where X is the % I in the presence of IGF-1IR ligand. The apparent binding affinity (presented as IC₅₀) was calculated by fitting the % inhibition data with a non-linear 4-parameter inhibition equation using GraphPad Prism (GraphPad Software, LLC; version 9.4.1).

### Mannose 6-Phosphate Ligand Binding Data

| | | TCF INH |
|---|---|---|
| | | (HTRF) |
| Compound | | IC₅₀ |
| ID | Structure | (μM) |
| A1 | 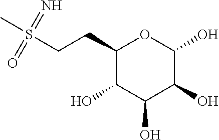 [SMILES: CS(=N)(=O)CC[C@H]1O[C@H](O)[C@@H](O)[C@@H](O)[C@@H]1O] | *** |
| A2 | 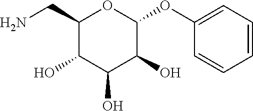 [SMILES: NC[C@H]1O[C@H](Oc2ccccc2)[C@@H](O)[C@@H](O)[C@@H]1O] | ** |
| A3 | 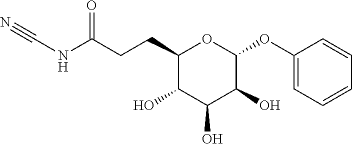 [SMILES: N#CNC(=O)CC[C@H]1O[C@H](Oc2ccccc2)[C@@H](O)[C@@H](O)[C@@H]1O] | ** |
| A4 | 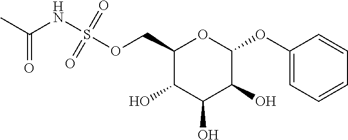 [SMILES: CC(=O)NS(=O)(=O)OC[C@H]1O[C@H](Oc2ccccc2)[C@@H](O)[C@@H](O)[C@@H]1O] | * |
| A7 | 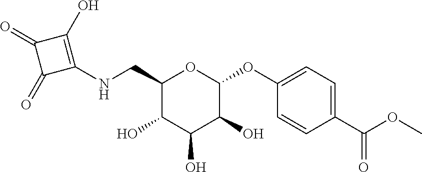 [SMILES: COC(=O)c1ccc(O[C@H]2O[C@H](CNc3c(O)c(=O)c3=O)[C@@H](O)[C@H](O)[C@@H]2O)cc1] | * |
| A8 | 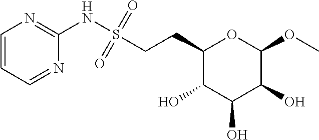 [SMILES: CO[C@@H]1O[C@H](CCS(=O)(=O)Nc2ncccn2)[C@@H](O)[C@H](O)[C@@H]1O] | ** |
| A9 | 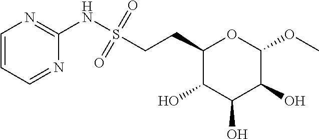 [SMILES: CO[C@H]1O[C@H](CCS(=O)(=O)Nc2ncccn2)[C@@H](O)[C@H](O)[C@@H]1O] | * |
| A11 | 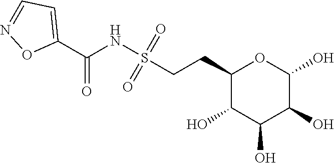 [SMILES: O=C(NS(=O)(=O)CC[C@H]1O[C@H](O)[C@@H](O)[C@@H](O)[C@@H]1O)c1ccno1] | ** |
| A12 | 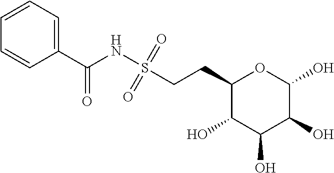 [SMILES: O=C(NS(=O)(=O)CC[C@H]1O[C@H](O)[C@@H](O)[C@@H](O)[C@@H]1O)c1ccccc1] | * |
| A13 | 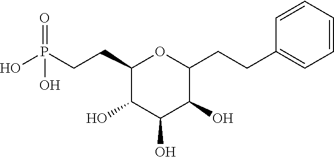 [SMILES: O=P(O)(O)CC[C@H]1OC(CCc2ccccc2)[C@@H](O)[C@@H](O)[C@@H]1O] | *** |
| A14 | 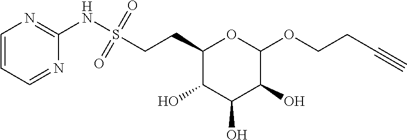 [SMILES: C#CCCOC1O[C@H](CCS(=O)(=O)Nc2ncccn2)[C@@H](O)[C@H](O)[C@@H]1O] | * |
| A15 | 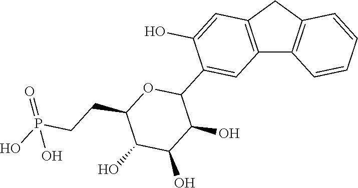 [SMILES: O=P(O)(O)CC[C@H]1OC(c2cc3c(cc2O)Cc2ccccc2-3)[C@@H](O)[C@@H](O)[C@@H]1O] | *** |
| A16 | 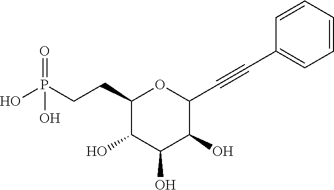 [SMILES: O=P(O)(O)CC[C@H]1OC(C#Cc2ccccc2)[C@@H](O)[C@@H](O)[C@@H]1O] | *** |
| A17 | 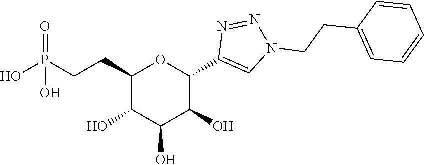 [SMILES: O=P(O)(O)CC[C@H]1O[C@H](c2cn(CCc3ccccc3)nn2)[C@@H](O)[C@@H](O)[C@@H]1O] | *** |
| A18 | 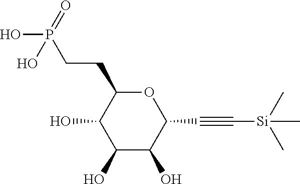 [SMILES: C[Si](C)(C)C#C[C@H]1O[C@H](CCP(=O)(O)O)[C@@H](O)[C@H](O)[C@@H]1O] | *** |
| A19 | 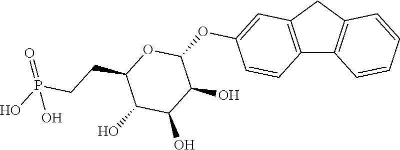 [SMILES: O=P(O)(O)CC[C@H]1O[C@H](Oc2ccc3c(c2)Cc2ccccc2-3)[C@@H](O)[C@@H](O)[C@@H]1O] | *** |
| A20 | 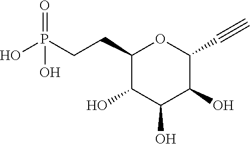 [SMILES: C#C[C@H]1O[C@H](CCP(=O)(O)O)[C@@H](O)[C@H](O)[C@@H]1O] | *** |
| A21 | 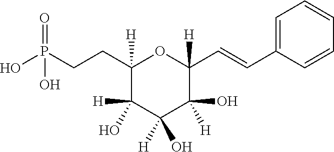 [SMILES: O=P(O)(O)CC[C@H]1O[C@H](/C=C/c2ccccc2)[C@@H](O)[C@@H](O)[C@@H]1O] | ** |
| A22 | 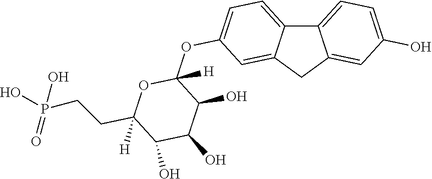 [SMILES: O=P(O)(O)CC[C@H]1O[C@H](Oc2ccc3c(c2)Cc2cc(O)ccc2-3)[C@@H](O)[C@@H](O)[C@@H]1O] | *** |
| A41 | 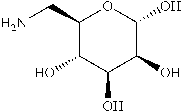 [SMILES: NC[C@H]1O[C@H](O)[C@@H](O)[C@@H](O)[C@@H]1O] | ** |
| A42 | 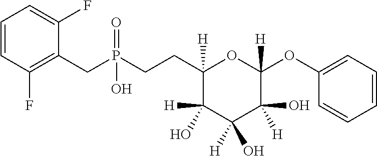 [SMILES: O=P(O)(CC[C@H]1O[C@H](Oc2ccccc2)[C@@H](O)[C@@H](O)[C@@H]1O)Cc1c(F)cccc1F] | *** |
| A43 | 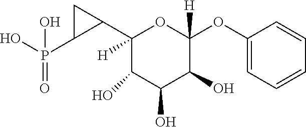 [SMILES: O=P(O)(O)C1CC1[C@H]1O[C@H](Oc2ccccc2)[C@@H](O)[C@@H](O)[C@@H]1O] | ** |
| A44 | 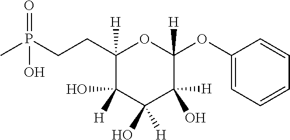 [SMILES: CP(=O)(O)CC[C@H]1O[C@H](Oc2ccccc2)[C@@H](O)[C@@H](O)[C@@H]1O] | ** |
| A45 | 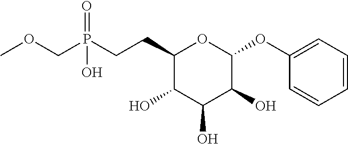 [SMILES: COCP(=O)(O)CC[C@H]1O[C@H](Oc2ccccc2)[C@@H](O)[C@@H](O)[C@@H]1O] | ** |
| A46 | 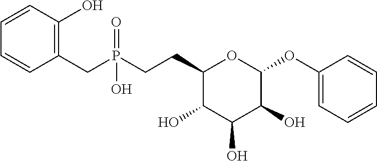 [SMILES: O=P(O)(CC[C@H]1O[C@H](Oc2ccccc2)[C@@H](O)[C@@H](O)[C@@H]1O)Cc1ccccc1O] | *** |
| A47 | 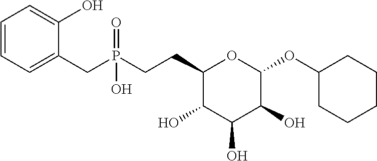 [SMILES: O=P(O)(CC[C@H]1O[C@H](OC2CCCCC2)[C@@H](O)[C@@H](O)[C@@H]1O)Cc1ccccc1O] | ** |
| A48 | 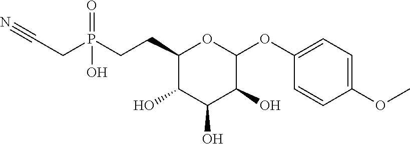 [SMILES: COc1ccc(OC2O[C@H](CCP(=O)(O)CC#N)[C@@H](O)[C@H](O)[C@@H]2O)cc1] | ** |
| A49 | 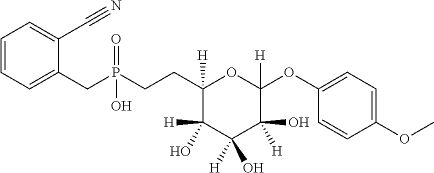 [SMILES: COc1ccc(OC2O[C@H](CCP(=O)(O)Cc3ccccc3C#N)[C@@H](O)[C@H](O)[C@@H]2O)cc1] | *** |
| A50 | 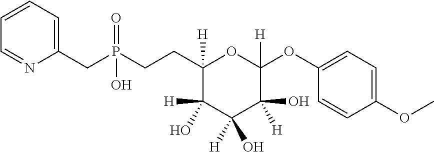 [SMILES: COc1ccc(OC2O[C@H](CCP(=O)(O)Cc3ccccn3)[C@@H](O)[C@H](O)[C@@H]2O)cc1] | *** |
| A51 | 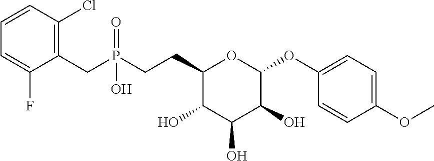 [SMILES: COc1ccc(O[C@H]2O[C@H](CCP(=O)(O)Cc3c(F)cccc3Cl)[C@@H](O)[C@H](O)[C@@H]2O)cc1] | *** |
| A52 | 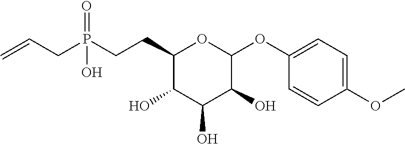 [SMILES: C=CCP(=O)(O)CC[C@H]1OC(Oc2ccc(OC)cc2)[C@@H](O)[C@@H](O)[C@@H]1O] | ** |
| A53 | 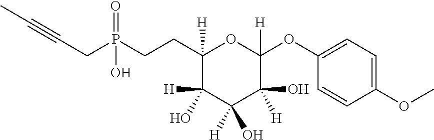 [SMILES: CC#CCP(=O)(O)CC[C@H]1OC(Oc2ccc(OC)cc2)[C@@H](O)[C@@H](O)[C@@H]1O] | ** |
| A54 | 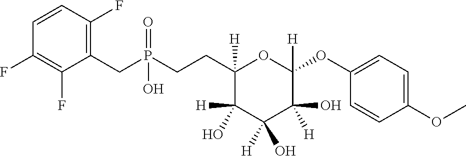 [SMILES: COc1ccc(O[C@H]2O[C@H](CCP(=O)(O)Cc3c(F)ccc(F)c3F)[C@@H](O)[C@H](O)[C@@H]2O)cc1] | *** |
| A55 | 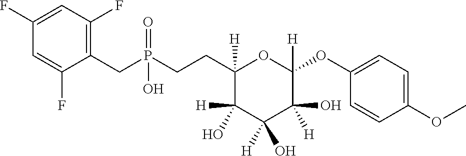 [SMILES: COc1ccc(O[C@H]2O[C@H](CCP(=O)(O)Cc3c(F)cc(F)cc3F)[C@@H](O)[C@H](O)[C@@H]2O)cc1] | ** |
| A56 | 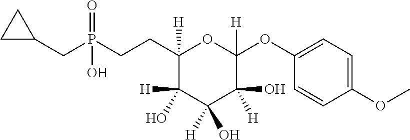 [SMILES: COc1ccc(OC2O[C@H](CCP(=O)(O)CC3CC3)[C@@H](O)[C@H](O)[C@@H]2O)cc1] | *** |
| A57 | 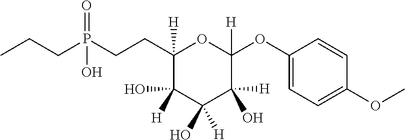 [SMILES: CCCP(=O)(O)CC[C@H]1OC(Oc2ccc(OC)cc2)[C@@H](O)[C@@H](O)[C@@H]1O] | *** |
| A58 | 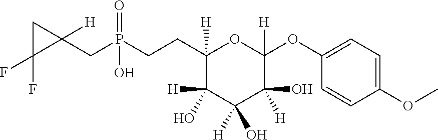 [SMILES: COc1ccc(OC2O[C@H](CCP(=O)(O)CC3CC3(F)F)[C@@H](O)[C@H](O)[C@@H]2O)cc1] | *** |
| A59 | 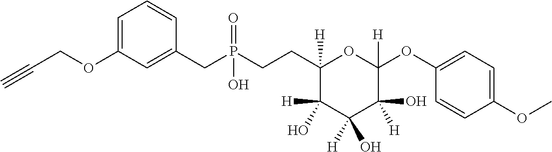 [SMILES: C#CCOc1cccc(CP(=O)(O)CC[C@H]2OC(Oc3ccc(OC)cc3)[C@@H](O)[C@@H](O)[C@@H]2O)c1] | *** |
| A60 | 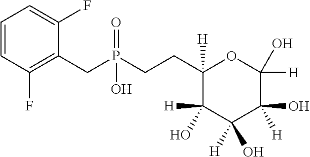 [SMILES: O=P(O)(CC[C@H]1OC(O)[C@@H](O)[C@@H](O)[C@@H]1O)Cc1c(F)cccc1F] | ** |
| A61 | 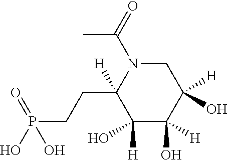 [SMILES: CC(=O)N1C[C@@H](O)[C@@H](O)[C@H](O)[C@H]1CCP(=O)(O)O] | ** |
| A62 | 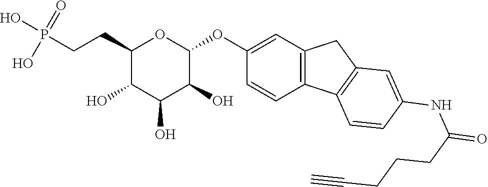 [SMILES: C#CCCCC(=O)Nc1ccc2c(c1)Cc1cc(O[C@H]3O[C@H](CCP(=O)(O)O)[C@@H](O)[C@H](O)[C@@H]3O)ccc1-2] | |
| A63 | 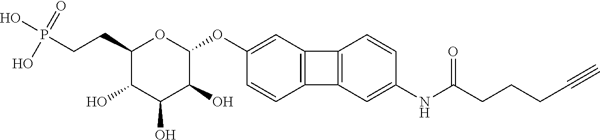 [SMILES: C#CCCCC(=O)Nc1ccc2c(c1)-c1ccc(O[C@H]3O[C@H](CCP(=O)(O)O)[C@@H](O)[C@H](O)[C@@H]3O)cc1-2] | |
| A64 | 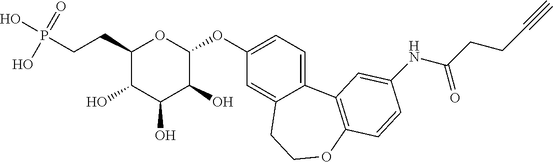 [SMILES: C#CCCC(=O)Nc1ccc2c(c1)-c1ccc(O[C@H]3O[C@H](CCP(=O)(O)O)[C@@H](O)[C@H](O)[C@@H]3O)cc1CCO2] | |
| A65 | 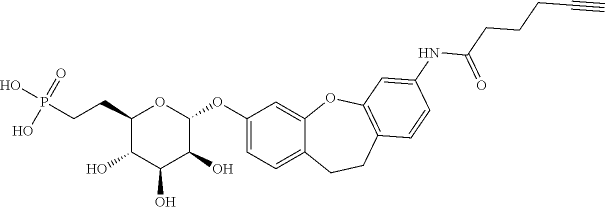 [SMILES: C#CCCCC(=O)Nc1ccc2c(c1)Oc1cc(O[C@H]3O[C@H](CCP(=O)(O)O)[C@@H](O)[C@H](O)[C@@H]3O)ccc1CC2] | |
| A66 | 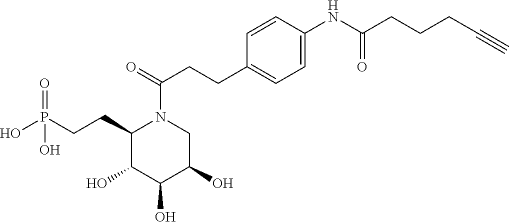 [SMILES: C#CCCCC(=O)Nc1ccc(CCC(=O)N2C[C@@H](O)[C@@H](O)[C@H](O)[C@H]2CCP(=O)(O)O)cc1] | |
| A67 | 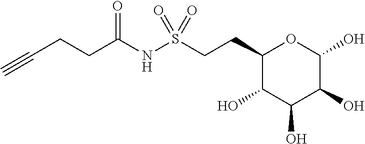 [SMILES: C#CCCC(=O)NS(=O)(=O)CC[C@H]1O[C@H](O)[C@@H](O)[C@@H](O)[C@@H]1O] | |
| A68 | 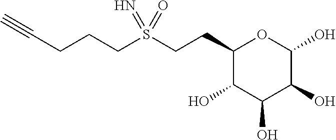 [SMILES: C#CCCCS(=N)(=O)CC[C@H]1O[C@H](O)[C@@H](O)[C@@H](O)[C@@H]1O] | |
| A69 | 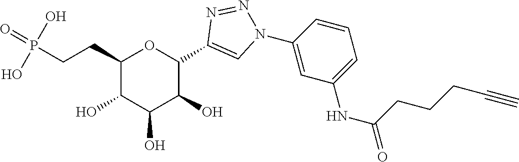 | |
| A70 | 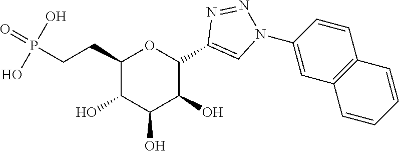 | |
| A71 | 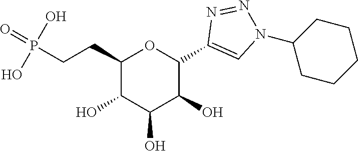 | |
| A72 | 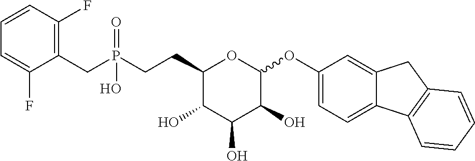 [SMILES: O=P(O)(CC[C@H]1OC(Oc2ccc3c(c2)Cc2ccccc2-3)[C@@H](O)[C@@H](O)[C@@H]1O)Cc1c(F)cccc1F] | |
| A73 | 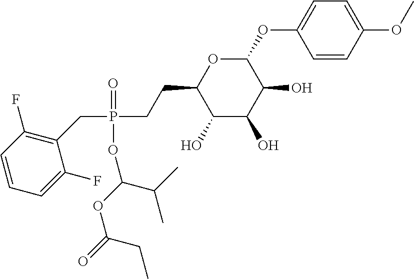 [SMILES: CCC(=O)OC(OP(=O)(CC[C@H]1O[C@H](Oc2ccc(OC)cc2)[C@@H](O)[C@@H](O)[C@@H]1O)Cc1c(F)cccc1F)C(C)C] | |
| A74 | 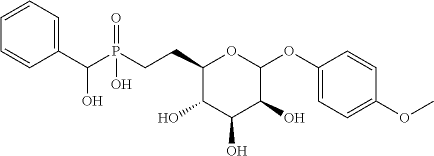 [SMILES: COc1ccc(OC2O[C@H](CCP(=O)(O)C(O)c3ccccc3)[C@@H](O)[C@H](O)[C@@H]2O)cc1] | *** |
| A75 | 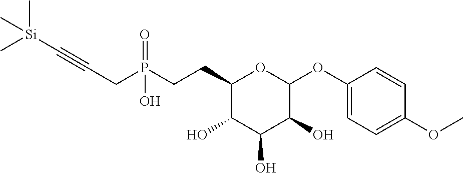 | ** |
| A76 | 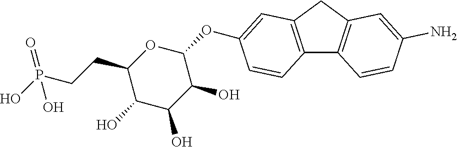 [SMILES: Nc1ccc2c(c1)Cc1cc(O[C@H]3O[C@H](CCP(=O)(O)O)[C@@H](O)[C@H](O)[C@@H]3O)ccc1-2] | *** |
| A77 | 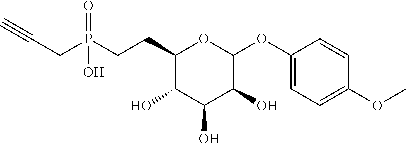 [SMILES: C#CCP(=O)(O)CC[C@H]1OC(Oc2ccc(OC)cc2)[C@@H](O)[C@@H](O)[C@@H]1O] | ** |
| A78 | 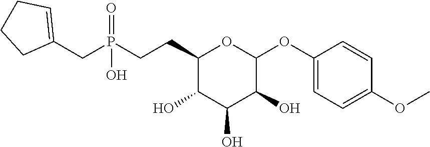 [SMILES: COc1ccc(OC2O[C@H](CCP(=O)(O)CC3=CCCC3)[C@@H](O)[C@H](O)[C@@H]2O)cc1] | *** |
| A79 | 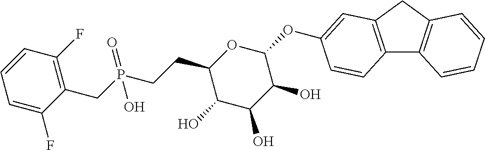 [SMILES: O=P(O)(CC[C@H]1O[C@H](Oc2ccc3c(c2)Cc2ccccc2-3)[C@@H](O)[C@@H](O)[C@@H]1O)Cc1c(F)cccc1F] | *** |
| A80 | 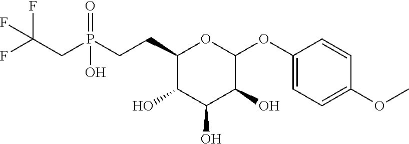 [SMILES: COc1ccc(OC2O[C@H](CCP(=O)(O)CC(F)(F)F)[C@@H](O)[C@H](O)[C@@H]2O)cc1] | ** |
| A81 | 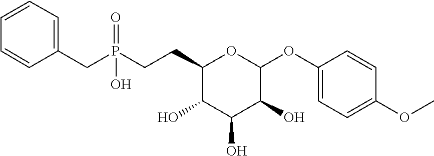 [SMILES: COc1ccc(OC2O[C@H](CCP(=O)(O)Cc3ccccc3)[C@@H](O)[C@H](O)[C@@H]2O)cc1] | *** |
| A82 | 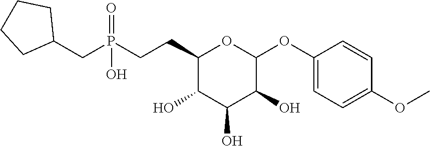 [SMILES: COc1ccc(OC2O[C@H](CCP(=O)(O)CC3CCCC3)[C@@H](O)[C@H](O)[C@@H]2O)cc1] | *** |
| A83 | 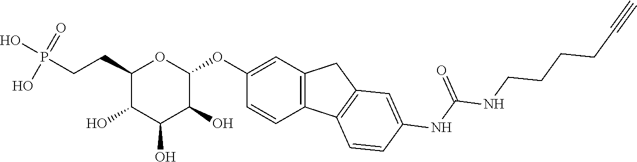 [SMILES: C#CCCCCNC(=O)Nc1ccc2c(c1)Cc1cc(O[C@H]3O[C@H](CCP(=O)(O)O)[C@@H](O)[C@H](O)[C@@H]3O)ccc1-2] | *** |
| A84 | 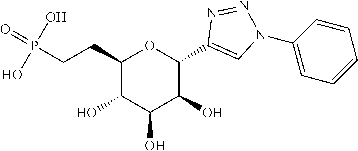 [SMILES: O=P(O)(O)CC[C@H]1O[C@H](c2cn(-c3ccccc3)nn2)[C@@H](O)[C@@H](O)[C@@H]1O] | *** |
| A85 | 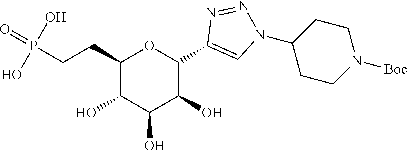 | *** |
| A86 | 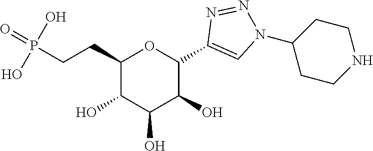 | *** |

***is IC₅₀ <10 μM
**is 10 μM <IC₅₀ < 100 μM
*is 100 μM < IC₅₀ < 300 μM

### Example 2. HTRF-based ternary complex formation assay for hIGF-IIR targeting Extracellular Protein Degraders

The ternary complex formation assay is designed to identify and determine the potency of Extracellular Protein Degrader molecules which can induce the formation of ternary complex between the Extracellular Protein Degrader hIGF-IIR. Titrating Extracellular Protein Degrader to fixed amount of hIGF-IIR results in a bell-shaped curve (except molecules whose binary complex affinity between Extracellular Protein Degrader and hIGF-IIR). The peak mean represents Extracellular Protein Degrader concentration at which maximum ternary complex is formed. Too much or too little Extracellular Protein Degrader leads to decreased ternary complex formation due to formation of binary complex (Hook effect). Dose response of Extracellular Protein Degrader compounds (16-point 3-fold dilution series, 100X) were prepared by Echo Acoustic Liquid Handler (Beckman Coulter Life Sciences) using 384-well LDV microplate. Then 200 nL of the solutions were transferred to an OptiPlate 384-well microplate (PerkinElmer, 6007290). To the assay plate, 10 μL working solution containing 60 nM of Biotinylated-IgG1 (Viva 20211108) and 60 nM of His-tagged hIGF-IIR (R&D Systems, 6418-GR) in assay buffer 50 mM Tris, pH 7.5, 150 mM NaCl, 50 mM CaCl₂, 0.01% Tween-20 were added. The plate was incubated for 60 min at room temperature with gentle shaking to allow molecular interaction to reach equilibration. Following this, 10 μL of HTRF detection solution containing 15 nM Streptavidin-d2 (PerkinElmer, 610SADLA) and 0.7 nM MAb Anti 6HIS—Tb cryptate (PerkinElmer, 61HISTLA) in PPI Terbium detection buffer (PerkinElmer, 61DB10RDF) were added. The assay plate was incubated at room temperature with gentle shaking for 60 min to allow HTRF signal to develop. The plate was gently spun on a centrifuge then placed on an EnVision plate reader (Perkin Elmer, EnVision 2104) to read fluorescence ratio of 665 nm/615 nm. Data were analyzed by fitting with Bell-shaped non-linear regression model using GraphPad Prism (GraphPad Software, LLC; version 9.4.1). Mean fluorescence intensity is graphed versus compound concentration and the EC50 for TCF is calculated from the inflection point of the curve (GraphPad).

### Compound Binding Data

| | | TCF |
|---|---|---|
| Com- | | (HTRF) |
| pound | | EC₅₀ |
| ID | Structure | (nM) |
| Com- pound 1 | 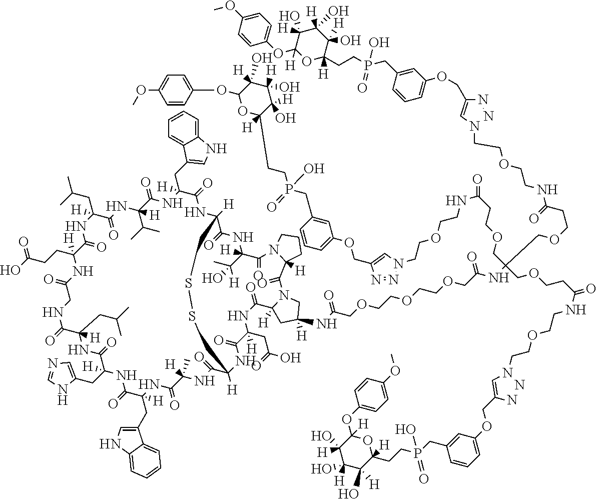 | *** |
| Com- pound 2 | 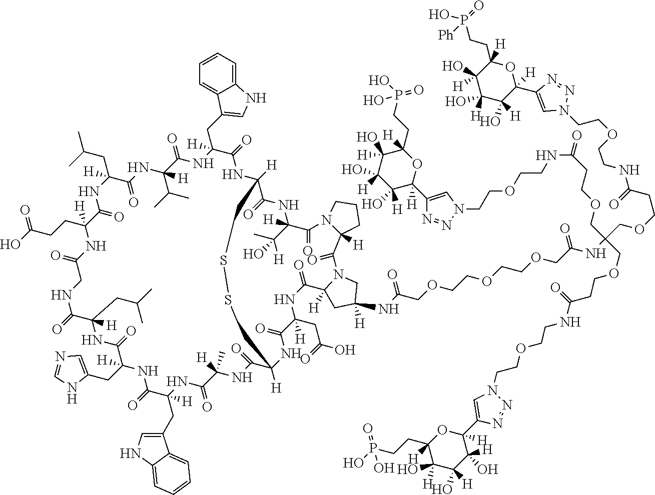 | *** |
| Com- pound 5 | 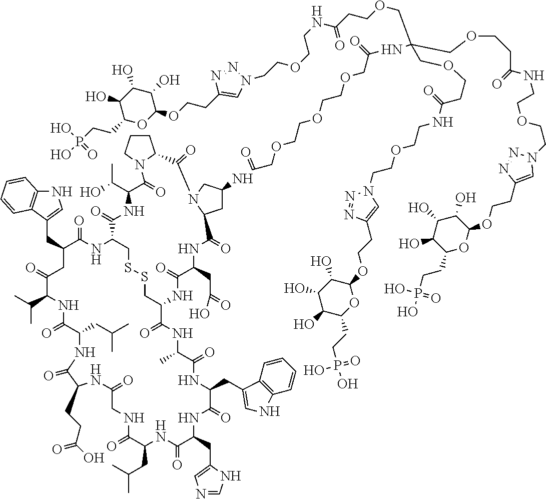 | *** |
| Com- pound 6 | 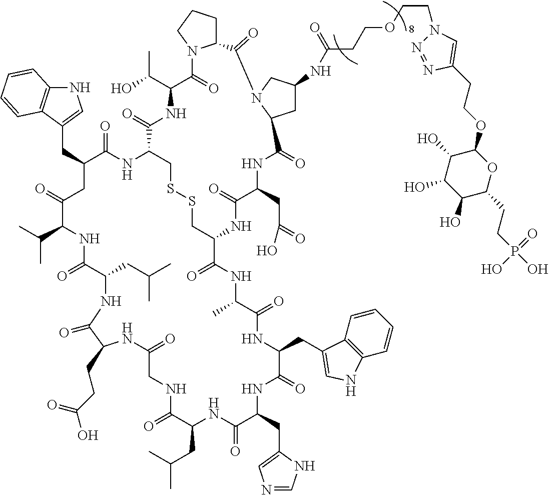 | *** |
| Com- pound 8 | 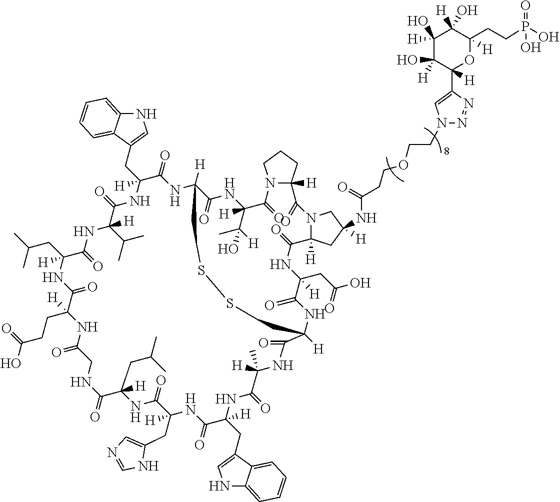 | *** |
| Com- pound 9 | 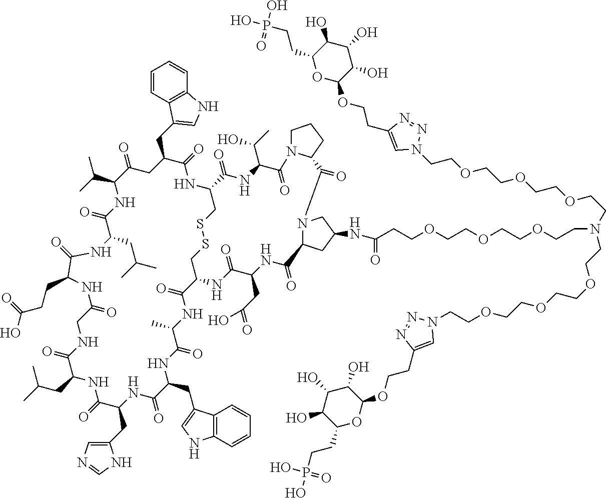 [SMILES: CC(C)C[C@@H]1NC(=O)[C@H](Cc2cnc[nH]2)NC(=O)[C@H](Cc2c[nH]c3ccccc23)NC(=O)[C@H](C)NC(=O)[C@@H]2CSSC[C@H](NC(=O)[C@H](Cc3c[nH]c4ccccc34)CC(=O)[C@H](C(C)C)NC(=O)[C@H](CC(C)C)NC(=O)[C@H](CCC(=O)O)NC(=O)CNC1=O)C(=O)N[C@@H]([C@@H](C)O)C(=O)N1CCC[C@@H]1C(=O)N1C[C@@H](NC(=O)CCOCCOCCOCCN(CCOCCOCCOCCn3cc(CCO[C@H]4O[C@H](CCP(=O)(O)O)[C@@H](O)[C@H](O)[C@@H]4O)nn3)CCOCCOCCOCCn3cc(CCO[C@H]4O[C@H](CCP(=O)(O)O)[C@@H](O)[C@H](O)[C@@H]4O)nn3)C[C@H]1C(=O)N[C@@H](CC(=O)O)C(=O)N2] | *** |
| Com- pound 10 | 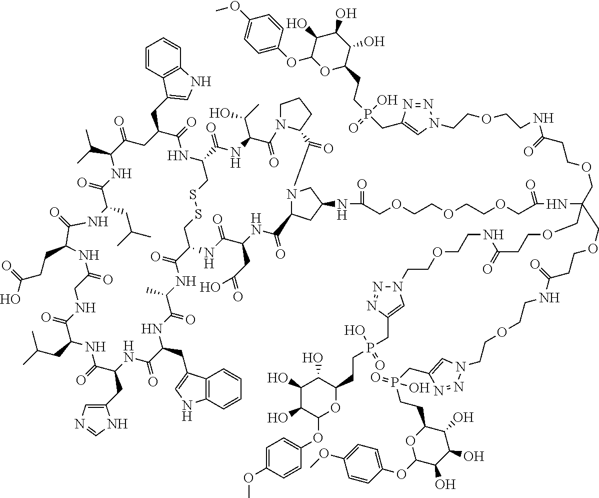 | *** |
| Com- pound 11 | 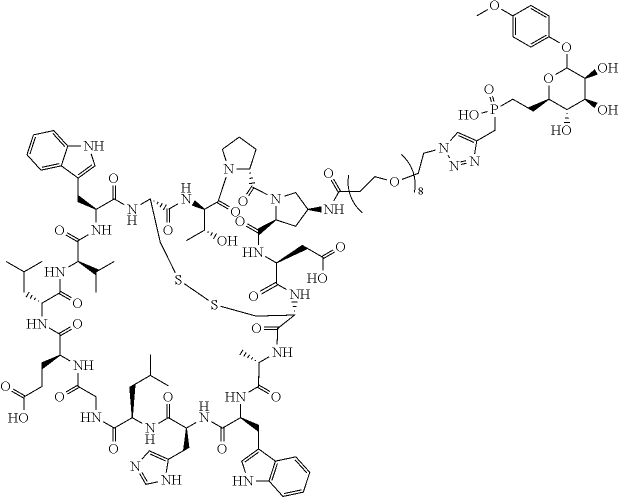 | *** |
| Com- pound 12 | 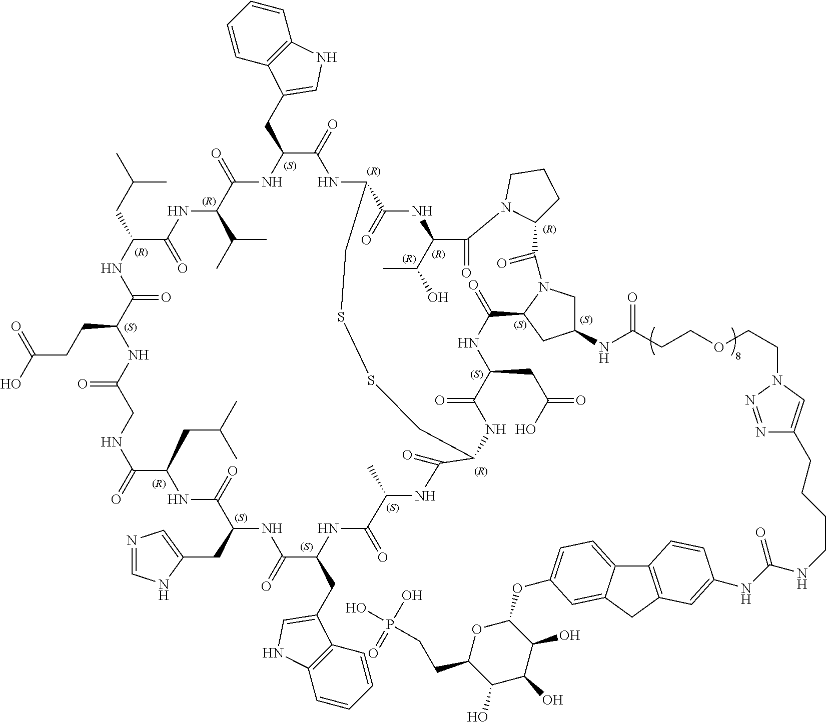 | *** |

<50 nM = ***
50 nM < IC50 < 250 nM = **
>250 nM = *

### Example 3. Dmpk Study

The dose formulation of IgG at 200 mg/kg was administered via IV injection to male SD rats, and Compound 5 was administered via IV injection an hour later (after IgG dosing). About 0.2 mL of blood was collected via jugular vein at each time point and was transferred into pre-cooled low binding EP tubes containing 4 μL of K2-EDTA (0.5 M) as anti-coagulant placed on wet ice. After blood was collected, the samples were processed for plasma by centrifugation at approximately 4° C., 3200 g for 10 minutes, the plasma was obtained within 30 minutes of blood collection. Then the plasma was transferred to two clean low binding EP tubes (50 μL each, one for PK, one for PD) and quickly frozen over dry ice and kept below −60° C. or lower for LC-MS/MS analysis. Plasma concentration versus time data was plotted in graph and analyzed by non-compartmental approaches using the Phoenix WinNonlin 6.3 software program. Related PK parameters were calculated according to dosing route, e.g. CL, Vdss and C₀ for intravenous administration, Cₘₐₓ, Tₘₐₓ or % F for extravascular administration, and T_{1/2}, AUC₍₀₋₁₎, AUC_{(0-inf)}, MRT₍₀₋₁₎, MRT_{(0-inf)} for all routes. The quantification of human IgG concentration in animal plasma was conducted using human IgG ELISA kit. Plotted pharmacokinetic and pharmacodynamic data can be found in FIG. 10, FIG. 11, FIG. 12, and FIG. 13.

The pharmacokinetic data in FIG. 9 showing the plasma concentration of Compound 12 over time was performed using a similar protocol to that of Compound 5 described above.

### EXAMPLE 4. TERNARY COMPLEX FORMATION (TCF) IN K562

K562 cells were seeded in 96-well assay plate using K562 cell medium, then compounds and AF488-labeled human IgG (final concentration of 300 nM) were added at 4° C. and incubated for 1 hour. Afterwards, the cells were washed once with 1×PBS+0.1% BSA (Cold) and centrifuged at 1000 rpm for 5 minutes. Next, cells were re-suspended in 150 μL 1×PBS+0.1% BSA and ran flow cytometry on iQue® Screener PLUS.

### Ternary Complex Assay K562 Cells

| Compound | EC₅₀ (μM) |
|---|---|
| Compound 2 | *** |
| Compound 5 | ** |
| Compound 8 | *** |
| Compound 12 | *** |

<50 nM = ***
50 nM < IC₅₀ < 250 nM = **

### EXAMPLE 5. ASSAY PROTOCOL OF UPTAKE AND DEGRADATION IN K562 CELLS

On day one, cells were harvested, counted, and seeded 1 million/well in 6 well plates for each cell line. On day two, cells were treated with compounds and IgG (Sigma, Cat #14506 dissolved in medium). Treated cells were washed three times with PBS, followed by addition of 200 μL of RIPA buffer (+HALT protease and phosphatase inhibitors) per well. Lysates were left on ice for 5 minutes then were collected into microcentrifuge tubes using a cell scraper. Lysates were then kept on a shaker for 30-60 minutes at 4° C. followed by centrifugation at 14,000 rpm at 4° C. for 15 minutes. Then, supernatants were transferred to fresh tubes and total protein concentration of each sample was determined using Pierce BCA kit. Next, 25 ug total protein of each sample was resolved on a 4-20% gels and transferred to PVDF membranes. Membranes were blocked in TBST+5% milk (made fresh) for one hour while shaking at room temperature. Then, the first antibody was added in TBST+5% milk at 4° C. overnight: a) Rabbit anti-human IgG, Abcam; Cat #ab181236-1:1000, b) anti-β-actin, Cell signaling Tech, Cat #3700S)-1:3000. On the next day, membranes were washed with TBST 10 minutes each for three times. Later, the second antibody was added in TBST+5% milk at room temperature over one hour: a) IRDye 800CW Goat anti Rabbit IgG, Licor, Cat #926-32211)-1:10,000; b) IRDye 680RD Goat anti Mouse IgG (Licor Cat #926-68078)-1:10,000. Then, membranes were washed with TBST 10 minutes each for three times. The results were detected by Licor. Data from the uptake and degradation assay is shown in FIG. 14, FIG. 15, FIG. 16, and FIG. 17.

### Preparation of imino(methyl) (2-((2R,3S,4S,5S,6S)-3,4,5,6-tetrahydroxytetrahydro-2H-pyran-2-yl)ethyl)-16-sulfanone (A1)

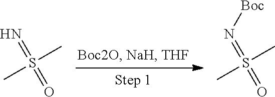

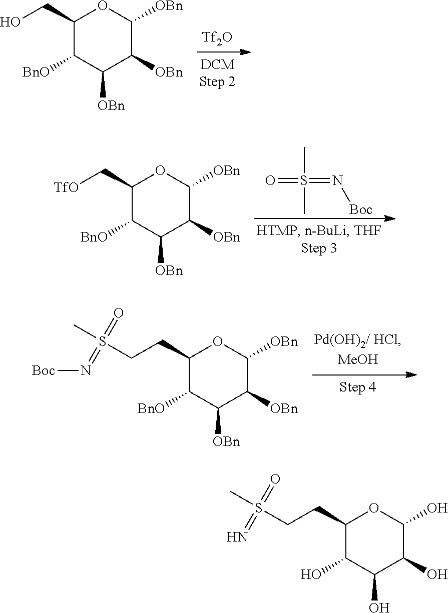 [SMILES: "Tf2O" ; OC[C@H]1O[C@H](OCc2ccccc2)[C@@H](OCc2ccccc2)[C@@H](OCc2ccccc2)[C@@H]1OCc1ccccc1 ; O=S(=O)(OC[C@H]1O[C@H](OCc2ccccc2)[C@@H](OCc2ccccc2)[C@@H](OCc2ccccc2)[C@@H]1OCc1ccccc1)C(F)(F)F ; CC(C)(C)OC(=O)N=S(C)(C)=O ; CC(C)(C)OC(=O)N=S(C)(=O)CC[C@H]1O[C@H](OCc2ccccc2)[C@@H](OCc2ccccc2)[C@@H](OCc2ccccc2)[C@@H]1OCc1ccccc1 ; CS(=N)(=O)CC[C@H]1O[C@H](O)[C@@H](O)[C@@H](O)[C@@H]1O]

Step 1: To a solution of iminodimethyl-16-sulfanone (1 g, 10.7 mmol) and Boc₂O (4.68 g, 21.4 mmol) in THF (10 mL) was added NaH (0.46 g, 19.3 mmol). The reaction mixture was stirred at room temperature overnight. The mixture was quenched by water (50 mL), then extracted with EA (10 mL×3). The combined organic layers were washed with brine (20 mL×2), dried with anhydrous Na₂SO₄, filtered and concentrated. The residue was purified by flash chromatography (PE/EA=1:1) to give tert-butyl(dimethyl(oxo)-16-sulfanylidene) carbamate (1.5 g, 72% yield) as a white solid. LC-MS (ESI) found: 194 [M+H]⁺.

Step 2: To a solution of ((2R,3R,4S,5S,6S)-3,4,5,6-tetrakis(benzyloxy)tetrahydro-2H-pyran-2-yl)methanol (1.5 g, 2.8 mmol) in DCM (12 mL) was added Tf₂O (1.14 g, 4.0 mmol) at −30° C. slowly. The mixture was stirred at −30° C. for 1 hour. The reaction mixture was quenched with water and extracted with DCM. The combined organic layers were dried with anhydrous Na₂SO₄, filtered and concentrated. The residue was purified by flash chromatography (PE/EA=10:1) to give ((2R,3R,4S,5S,6S)-3,4,5,6-tetrakis(benzyloxy)tetrahydro-2H-pyran-2-yl)methyltrifluoromethane sulfonate (1.6 g, 85% yield) as a colorless oil.

Step 3: Under N₂ atmosphere, at 0° C., to a solution of HTMP (203 mg, 1.4 mmol) in THF (5 mL) was added n-BuLi (0.5 mL, 1.2 mmol, 2.5N). The reaction was stirred at 0° C. for 10 min, then cooled to −78° C. To the above mixture, tert-butyl(dimethyl(oxo)-16-sulfanylidene) carbamate (198 mg, 1.0 mmol) was added. The resulting mixture was stirred at −78° C. for 10 min and then warmed to −10° C. for 30 min and then cooled to −78° C. To the above mixture, ((2R,3R,4S,5S,6S)-3,4,5,6-tetrakis(benzyloxy)tetrahydro-2H-pyran-2-yl)methyl trifluoromethanesulfonate (500 mg, 0.7 mmol) was added slowly. The reaction was stirred at room temperature for overnight. The mixture was quenched by saturated aq. solution of NH₄Cl (10 mL) and extracted with EA (10 mL×3). The combined organic layers were washed with brine (20 mL×3), dried with anhydrous Na₂SO₄, filtered and concentrated. The residue was purified by prep-TLC (EA/PE=1:1) to give tert-butyl(methyl(oxo) (2-((2R,3R,4S,5S,6S)-3,4,5,6-tetrakis(benzyloxy)tetrahydro-2H-pyran-2-yl)ethyl)-16-sulfanylidene) carbamate (300 mg, 40%) as a white solid. LC-MS (ESI) found: 716 [M+H]⁺.

Step 4: Under hydrogen atmosphere, a suspension of tert-butyl(methyl(oxo) (2-((2R,3R,4S,5S,6S)-3,4,5,6-tetrakis(benzyloxy)tetrahydro-2H-pyran-2-yl)ethyl)-16-sulfanylidene) carbamate (50 mg, 0.07 mmol) in aq. HCl (1 mL, 1 N) and MeOH (3 mL) was stirred at room temperature for overnight. The reaction mixture was filtered and concentrated to give imino(methyl) (2-((2R,3S,4S,5S,6S)-3,4,5,6-tetrahydroxytetrahydro-2H-pyran-2-yl)ethyl)-16-sulfanone (13 mg, 73% yield) as a brown solid. LC-MS (ESI) found: 256 [M+H]⁺. ¹H NMR (400 MHz, CD₃OD) δ 5.07 (s, 1H), 4.10-3.96 (m, 2H), 3.95-3.79 (m, 2H), 3.79-3.60 (m, 4H), 3.54-3.43 (m, 1H), 2.55-2.37 (m, 1H), 2.21-2.06 (m, 1H).

### Preparation of (2R,3S,4S,5S,6R)-2-(aminomethyl)-6-phenoxytetrahydro-2H-pyran-3,4,5-triol (A2)

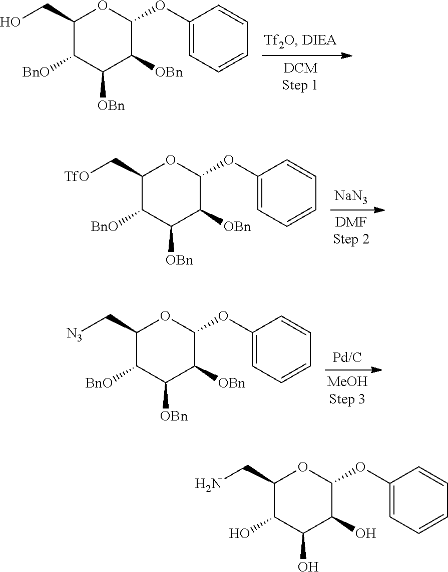

Step 1: To a solution of ((2R,3R,4S,5S,6R)-3,4,5-tris(benzyloxy)-6-phenoxytetrahydro-2H-pyran-2-yl)methanol (1 g, 2 mmol) and DIEA (740 mg, 6 mmol) in DCM (20 mL) was added Tf₂O (930 mg, 3 mmol) at −30° C. and the mixture was stirred at −30° C. for 1 h. After completion, the reaction mixture was quenched with NH₄Cl and extracted with DCM, the organic layers were combined and dried over anhydrous Na₂SO₄, filtered and concentrated. The residue was purified by silica gel chromatography (PE/EA=1/1) to give ((2R,3R,4S,5S,6R)-3,4,5-tris(benzyloxy)-6-phenoxytetrahydro-2H-pyran-2-yl)methyl trifluoromethanesulfonate (1 g, 80% yield) as colorless oil, which was used for next step immediately.

Step 2: To a mixture of ((2R,3R,4S,5S,6R)-3,4,5-tris(benzyloxy)-6-phenoxytetrahydro-2H-pyran-2-yl)methyl trifluoromethanesulfonate (1 g, 1.5 mmol) in DMF (20 mL) was added NaN₃ (1 g, 15 mmol) at rt and stirred at 100° C. for 48 h. After completion, the mixture was quenched with water and extracted with EA, the organic layers were combined and dried over anhydrous Na₂SO₄, filtered and concentrated, the residue was purified by silica gel chromatography (PE/EA=1/1) to give (2R,3R,4S,5S,6R)-2-(azidomethyl)-3,4,5-tris(benzyloxy)-6-phenoxytetrahydro-2H-pyran (580 mg, 70% yield) as yellow oil.

Step 3: To a mixture of (2R,3R,4S,5S,6R)-2-(azidomethyl)-3,4,5-tris(benzyloxy)-6-phenoxytetrahydro-2H-pyran (200 mg, 0.4 mmol) in MeOH (10 mL) was added Pd/C (100 mg, 10% purity) at rt and the resulting mixture was stirred at rt for 16 h under hydrogen atmosphere. After completion, the mixture was filtered and concentrated to give (2R,3S,4S,5S,6R)-2-(aminomethyl)-6-phenoxytetrahydro-2H-pyran-3,4,5-triol (49 mg, 53% yield) as colorless gel. ¹H NMR (400 MHz, CD₃OD) δ 7.32 (s, 2H), 7.19-6.96 (m, 3H), 5.57 (s, 1H), 4.07 (s, 1H), 3.96 (d, J=7.1 Hz, 1H), 3.83 (s, 1H), 3.68 (s, 1H), 3.34 (d, J=15.5 Hz, 1H), 3.13 (s, 1H).

### Preparation of N-cyano-3-((2R,3S,4S,5S,6R)-3,4,5-trihydroxy-6-phenoxytetrahydro-2H-pyran-2-yl)propanamide (A3)

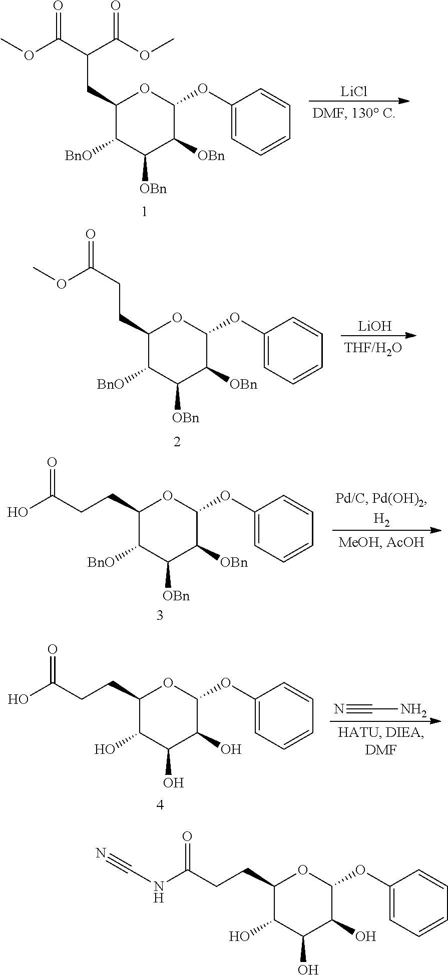

Step 1: The mixture of 1 (1.00 eq) and LiCL (2.00 eq) in DMF was stirred at 130° C. under N₂ overnight. The mixture was diluted with water, then extracted with EA, the organic phase was washed with brine, dried over anhydrous Na₂SO₄, filtered and concentrated, the residue was purified by silica gel chromatography (PE:EA=6:1) to afford 2 as a off-white solid. Chemical Formula: C₃₆H₃₈O₇. LCMS found: [M−H]⁻=581.

Step 2: To a mixture of 2 (1.00 eq) in THF and H₂O (4:1) was added lithium hydroxide (2.00 eq), the mixture was stirred at 25° C. overnight. The mixture was diluted with water, the pH of the aqueous was adjusted to 5 by HCl (1N), then extracted with EA, the organic layer was dried over anhydrous Na₂SO₄, filtered and concentrated, the residue was purified by silica gel chromatography (PE:EA=3:1) to afford 3 as a colorless oil. Chemical Formula: C₃₅H₃₆O₇, LCMS found: [M−H]⁻=567.

Step 3: To a mixture of 3 (1.00 eq) in MeOH was added Pd/C (10% purity), Pd(OH)₂ (10% purity) and AcOH (0.20 eq), the mixture was stirred at 25° C. under H₂ atmosphere (15 psi) overnight. The mixture was filtered and the filtrate was concentrated to afford 6 as a colorless oil. Chemical Formula: C₁₄H₁₈O₇. LCMS found: [M−H]⁻=297.

Step 4: The mixture of 4 (1.00 eq), HATU (1.50 eq), cyanamide (1.50 eq) and DIEA (3.00 eq) in DMF was stirred at 25° C. overnight. The mixture was concentrated under reduced pressure, the residue was purified by prep-HPLC (5-15% MeCN in water, 0.1% TFA) to afford A3 as a white solid. Chemical Formula: C₁₅H₁₈N₂O₆. ¹H NMR (400 MHz, CD₃OD): δ 7.33-7.26 (m, 2H), 7.08-6.97 (m, 3H), 5.45 (d, J=1.6 Hz, 1H), 4.00 (dd, J=3.2, 1.6 Hz, 1H), 3.87-3.82 (m, 1H), 3.56-3.48 (m, 2H), 2.43-2.30 (m, 1H), 2.18-2.08 (m, 2H), 1.76-1.68 (m, 1H).

### Preparation of ((2R,3S,4S,5S,6R)-3,4,5-trihydroxy-6-phenoxytetrahydro-2H-pyran-2-yl)methyl acetylsulfamate (A4)

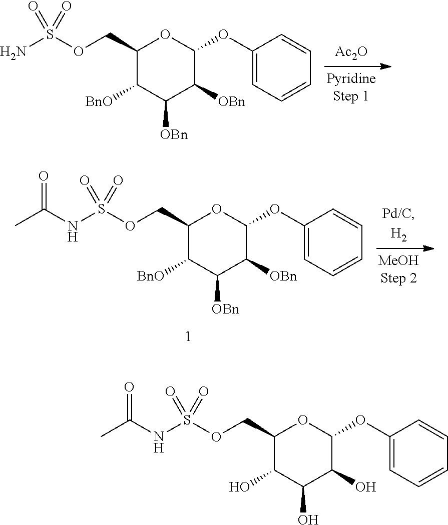 [SMILES: "Ac2O" ; NS(=O)(=O)OC[C@H]1O[C@H](Oc2ccccc2)[C@@H](OCc2ccccc2)[C@@H](OCc2ccccc2)[C@@H]1OCc1ccccc1 ; CC(=O)NS(=O)(=O)OC[C@H]1O[C@H](Oc2ccccc2)[C@@H](OCc2ccccc2)[C@@H](OCc2ccccc2)[C@@H]1OCc1ccccc1 ; CC(=O)NS(=O)(=O)OC[C@H]1O[C@H](Oc2ccccc2)[C@@H](O)[C@@H](O)[C@@H]1O]

Step 1: To a solution of ((2R,3R,4S,5S,6R)-3,4,5-tris(benzyloxy)-6-phenoxytetrahydro-2H-pyran-2-yl)methyl sulfamate (1.00 eq) in pyridine was added Ac₂O (2.00 eq) at 0° C. dropwise. The reaction was stirred at 25° C. for 2 h. The resulting mixture was concentrated in vacuo. The crude product was purified by flash chromatography (silica gel, 0˜40% EA in PE) to give 1 as colorless oil. Chemical Formula: C₃₅H₃₇NO₉S. LCMS found: [M+H]⁺=648.

Step 2: To a solution of 1 (1.00 eq) in MeOH was added Pd/C (10% purity), the mixture was degassed and purged with hydrogen several times, then stirred at 40° C. for 16 h under H₂ (15 psi) atmosphere. The mixture was filtered and the filtrate was concentrated in vacuo. The crude product was purified by prep-HPLC to give A4 as a white solid. Chemical Formula: C₁₄H₁₉NO₉S. LCMS found: [M+Na]⁺=400. ¹H NMR (400 MHz, CD₃OD): δ 7.32-7.27 (m, 2H), 7.11-7.07 (m, 2H), 7.02 (t, J=7.4 Hz, 1H), 5.43 (d, J=1.7 Hz, 1H), 4.46 (qd, J=11.0, 3.7 Hz, 2H), 4.01 (dd, J=3.4, 1.8 Hz, 1H), 3.89 (dd, J=9.3, 3.4 Hz, 1H), 3.83 (ddd, J=9.9, 5.2, 2.1 Hz, 1H), 3.77-3.70 (m, 1H), 2.07 (s, 3H).

### Preparation of N-((2-((2R,3S,4S,5S,6S)-6-(but-3-yn-1-yloxy)-3,4,5-trihydroxytetrahydro-2H-pyran-2-yl)ethyl)sulfonyl)acetamide (A5)

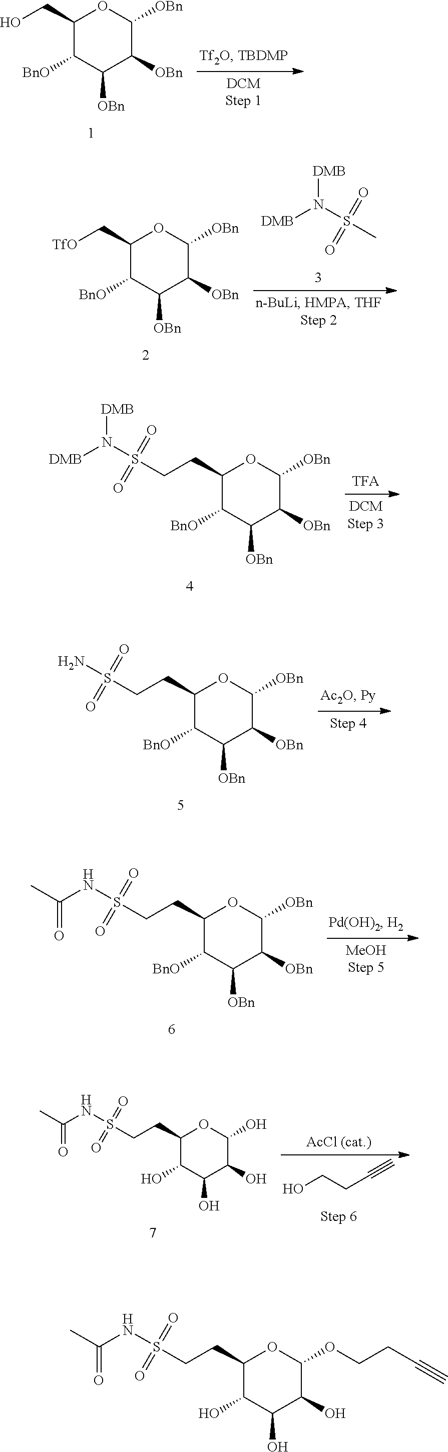

Step 1: To a solution of 1 (1.00 eq) and TBDMP (2.00 eq) in DCM (10 mL) was added Tf₂O (2.10 eq) at −30° C., the mixture was stirred at −30° C. for 0.5 hour. The mixture was quenched with NH₄Cl aq, then extracted with DCM, the organic layer was dried over anhydrous sodium sulfate, filtered and concentrated under reduced pressure, the residue was purified by silica gel column chromatography (0-10% ethyl acetate in petroleum ether) to afford 2 as a colorless oil. LCMS found: [M+H]⁺=673.

Step 2: To a solution of 3 (1.00 eq) and HMPA (3.00 eq) in THF (10 mL) was added n-BuLi (2.00 eq) at −78° C. The mixture was stirred at −78° C. for 1 hour, then 2 (1.00 eq) in THF (3 mL) was added slowly. The mixture was stirred at −78° C. for 1 hour, then quenched with NH₄Cl aq, extracted with EtOAc, the organic layer was dried over anhydrous sodium sulfate, filtered and concentrated under reduced pressure, the residue was purified by silica gel column chromatography (0-20% ethyl acetate in petroleum ether) to afford 4 as a colorless oil. LCMS found: [M+Na]⁺=940.

Step 3: To a solution of 4 (1.00 eq) in DCM (10 mL) was added TFA (4 mL) at 0° C. The mixture was stirred at 20° C. for 3 hours, then concentrated under reduced pressure. The residue was purified by reverse phase (C18, 5-90% MeCN in water, 0.2% TFA) to afford 5 as a colorless oil. LCMS found: [M+H]⁺=618.

Step 4: To a solution of 5 (1.00 eq) in Pyridine (3 mL) was added Ac₂₀ (2 mL) at 0° C. The mixture was stirred at 20° C. for 10 hours. The mixture was concentrated under reduced pressure. The residue was purified by reverse phase (C18, 5-90% MeCN in water, 0.2% TFA) to afford 6 as a colorless oil. LCMS found: [M+H]⁺=660.

Step 5: To a solution of 6 (1.00 eq) in MeOH (5 mL) and AcOH (0.1 mL) were added Pd/C (10% purity) and Pd(OH)₂ (10% purity), the mixture was degassed and purged with hydrogen several times, then stirred at 50° C. for 16 hours under hydrogen (15 psi) atmosphere. The mixture was filtered through a Celite pad, and the filtrate was concentrated to give 7 as colorless oil. LCMS found: [M+H]⁺=300.

Step 6: To a solution of 7 (1.00 eq) in but-3-yn-1-ol (2 mL) was added AcCl (0.03 mL), the mixture was stirred at 50° C. for 40 hours under nitrogen atmosphere. The mixture was concentrated under reduced pressure, and the residue was purified by mass-guided to give A5 as colorless oil. LCMS found: [M+H]⁺=352. ¹H NMR (400 MHz, CD₃OD): δ 4.75 (d, J=1.5 Hz, 1H), 3.83-3.72 (m, 2H), 3.69-3.60 (m, 2H), 3.58-3.51 (m, 2H), 3.51-3.39 (m, 2H), 2.51-2.42 (m, 2H), 2.36-2.20 (m, 2H), 2.07 (d, J=6.8 Hz, 3H), 1.98-1.78 (m, 1H).

### Preparation of tert-butyl 24-(4-(2-(((2S,3S,4S,5S,6R)-6-(2-(N-acetylsulfamoyl)ethyl)-3,4,5-trihydroxytetrahydro-2H-pyran-2-yl)oxy)ethyl)-1H-1,2,3-triazol-1-yl)-13,13-bis((3-((2-(2-(4-(2-(((2S,3S,4S,5S,6R)-6-(2-(N-acetylsulfamoyl)ethyl)-3,4,5-trihydroxytetrahydro-2H-pyran-2-yl)oxy)ethyl)-1H-1,2,3-triazol-1-yl)ethoxy)ethyl)amino)-3-oxopropoxy)methyl)-11,18-dioxo-3,6,9,15,22-pentaoxa-12,19-diazatetracosanoate (A6)

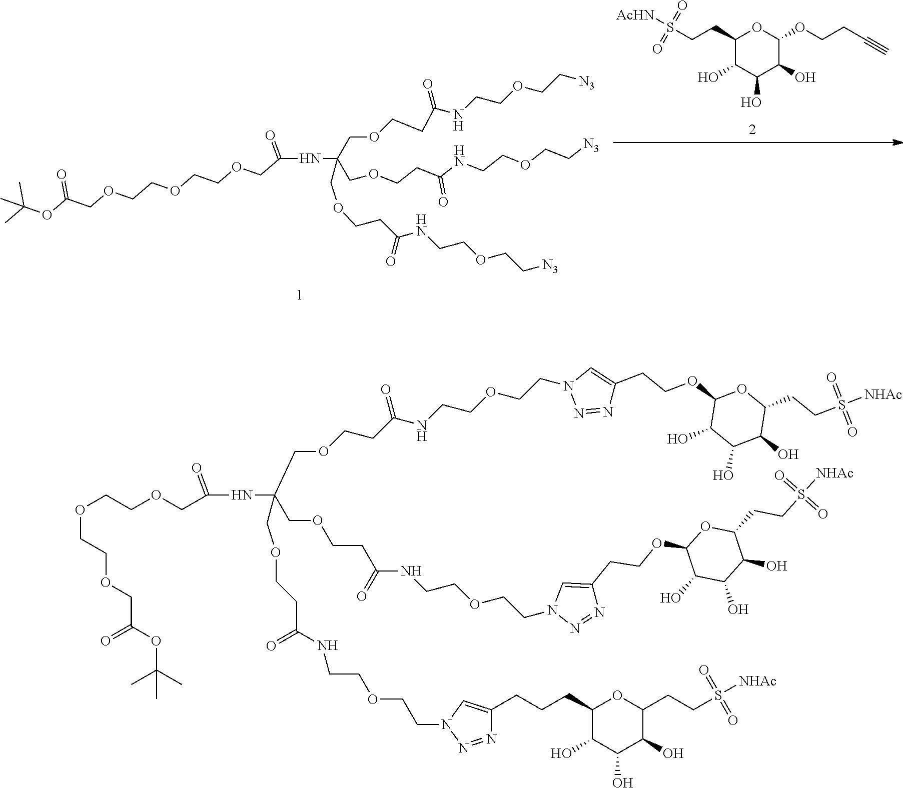 [SMILES: CC(C)(C)OC(=O)COCCOCCOCC(=O)NC(COCCC(=O)NCCOCCN=[N+]=[N-])(COCCC(=O)NCCOCCN=[N+]=[N-])COCCC(=O)NCCOCCN=[N+]=[N-] ; C#CCCO[C@H]1O[C@H](CCS(=O)(=O)NC(C)=O)[C@@H](O)[C@H](O)[C@@H]1O ; CC(=O)NS(=O)(=O)CCC1O[C@H](CCCc2cn(CCOCCNC(=O)CCOCC(COCCC(=O)NCCOCCn3cc(CCO[C@H]4O[C@H](CCS(=O)(=O)NC(C)=O)[C@@H](O)[C@H](O)[C@@H]4O)nn3)(COCCC(=O)NCCOCCn3cc(CCO[C@H]4O[C@H](CCS(=O)(=O)NC(C)=O)[C@@H](O)[C@H](O)[C@@H]4O)nn3)NC(=O)COCCOCCOCC(=O)OC(C)(C)C)nn2)[C@@H](O)[C@@H](O)[C@@H]1O]

To a solution of 1 (1.00 eq) and 2 (4.00 eq) in NMP (0.5 mL) was added Cu(MeCN)₄PF₆ (3.00 eq) at 20° C., the mixture was degassed and purged with nitrogen several times, then stirred at 20° C. for 2 hours under nitrogen atmosphere. The mixture was purified by reverse phase (C18, 5-50% MeCN in water, 0.2% TFA) to afford A6 as a colorless oil. LCMS found: [M+H]⁺=1988. ¹H NMR (400 MHz, CD₃OD): δ 7.90 (d, J=8.0 Hz, 3H), 4.91 (dd, J=16.1, 2.4 Hz, 3H), 4.72 (s, 1H), 4.58 (t, J=4.9 Hz, 6H), 4.28-4.18 (m, 1H), 4.10-3.83 (m, 16H), 3.80-3.73 (m, 3H), 3.69 (dd, J=11.6, 8.5 Hz, 22H), 3.59-3.49 (m, 12H), 3.38 (dt, J=9.1, 4.1 Hz, 11H), 3.00 (t, J=6.3 Hz, 6H), 2.43 (t, J=5.9 Hz, 2H), 2.36-2.28 (m, 2H), 2.24-2.11 (m, 2H), 2.12-2.01 (m, 9H), 1.89 (ddd, J=14.2, 10.2, 5.3 Hz, 2H), 1.47 (s, 9H).

### Preparation of methyl 4-(((3S,4S,5S,6R)-3,4,5-trihydroxy-6-(((2-hydroxy-3,4-dioxocyclobut-1-en-1-yl)amino)methyl)tetrahydro-2H-pyran-2-yl)oxy)benzoate (A7)

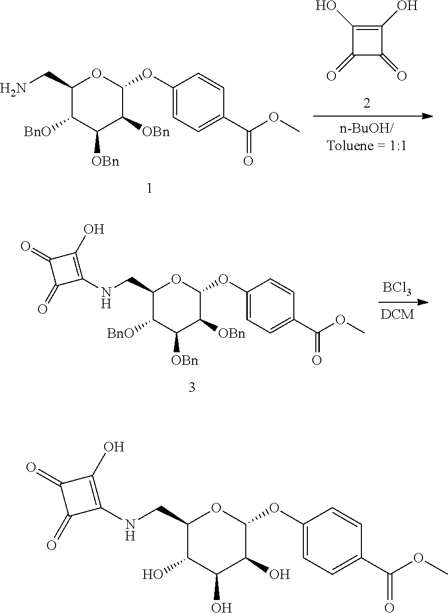 [SMILES: COC(=O)c1ccc(O[C@H]2O[C@H](CN)[C@@H](OCc3ccccc3)[C@H](OCc3ccccc3)[C@@H]2OCc2ccccc2)cc1 ; O=c1c(O)c(O)c1=O ; COC(=O)c1ccc(O[C@H]2O[C@H](CNc3c(O)c(=O)c3=O)[C@@H](OCc3ccccc3)[C@H](OCc3ccccc3)[C@@H]2OCc2ccccc2)cc1 ; "BCl3" ; COC(=O)c1ccc(O[C@H]2O[C@H](CNc3c(O)c(=O)c3=O)[C@@H](O)[C@H](O)[C@@H]2O)cc1]

Step 1: To a solution of 1 (1.00 eq) in n-BuOH (0.7 mL) and Toluene (0.7 mL) was added 2 (5.00 eq). The reaction mixture was stirred at 100° C. for 16 hours. The mixture was concentrated under reduced pressure, the residue was purified by reverse phase (C18, 5-90% MeCN in water, 0.3% TFA) to afford 3 as a colorless oil. LCMS found: [M+H]⁺=680.

Step 2: To a solution of 3 (1.00 eq) in DCM (3 mL) was added BCl₃ (5.00 eq) at −30° C. The reaction was stirred for at −30° C. for 20 min. The mixture was quenched by MeOH and then concentrated under reduced pressure, the residue was purified by prep-HPLC to afford A7 as a white solid. LCMS found: [M+H]⁺=410. ¹H NMR (400 MHz, CD₃OD): δ 7.92 (d, J=8.8 Hz, 2H), 7.12 (d, J=8.8 Hz, 2H), 5.58 (d, J=1.2 Hz, 1H), 4.02 (dd, J=3.2, 1.7 Hz, 1H), 3.89 (d, J=3.5 Hz, 1H), 3.87 (s, 3H), 3.81 (dd, J=14.0, 2.7 Hz, 1H), 3.73 (t, J=7.0 Hz, 1H), 3.67 (dd, J=15.9, 6.3 Hz, 1H), 3.62-3.56 (m, 1H).

### Preparation of N-(pyrimidin-2-yl)-2-((2R,3S,4S,5S,6R)-3,4,5-trihydroxy-6-methoxytetrahydro-2H-pyran-2-yl)ethane-1-sulfonamide (A8) and N-(pyrimidin-2-yl)-2-((2R,3S,4S,5S,6S)-3,4,5-trihydroxy-6-methoxytetrahydro-2H-pyran-2-yl)ethane-1-sulfonamide (A9)

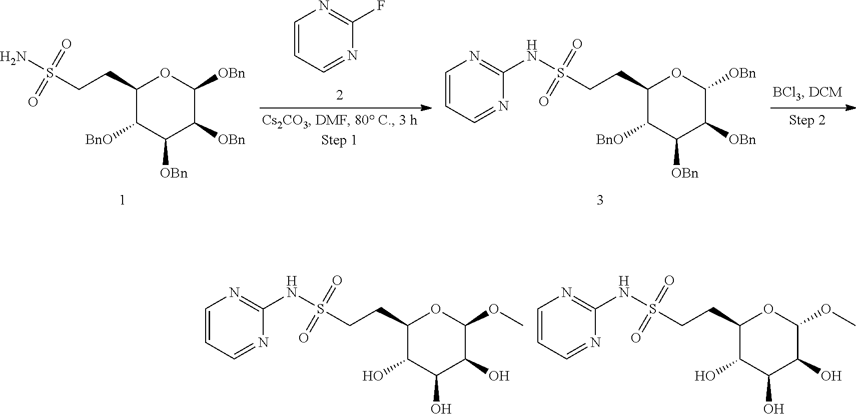

Step 1: To a solution of 1 (1.00 eq) in DMF (10 mL) was added Cs₂CO₃ (2.5 eq) and 2 (1.50 eq), the mixture was stirred at 80° C. for 3 hours. The mixture was diluted with water, extracted with ethyl acetate, the combined organic layers were washed with saturated solution of NH₄Cl, dried over anhydrous sodium sulfate, filtered and concentrated under reduced pressure. The residue was purified by silica gel column chromatography (10-50% EA in PE) to afford 3 as a white solid.

LCMS found: [M+H]⁺=696.

Step 2: To a solution of 3 (1.00 eq) in DCM (10 mL) was added BCl₃ (3.00 eq) at −20° C., the mixture was stirred at −20° C. for 1 h. The mixture was quenched with MeOH, then concentrated under reduced pressure. The residue was purified by prep-HPLC to afford A8 and A9 as a white solid. LCMS found: [M+H]⁺=350.

A16: ¹H NMR (400 MHz, CD₃OD): ò 8.54 (d, J=4.8 Hz, 2H), 7.07 (t, J=4.8 Hz, 1H), 4.34 (d, J=0.8 Hz, 1H), 3.85 (ddd, J=14.4, 10.4, 4.8 Hz, 1H), 3.82 (d, J=1.6 Hz, 1H), 3.70 (ddd, J=16.0, 10.4, 6.0 Hz, 1H), 3.45 (s, 3H), 3.39-3.34 (m, 2H), 3.26-2.97 (m, 1H), 2.45 (dddd, J=13.6, 10.4, 6.0, 2.8 Hz, 1H), 2.12-1.88 (m, 1H).

A17: ¹H NMR (400 MHz, CD₃OD): δ 8.54 (d, J=4.8 Hz, 2H), 7.06 (t, J=4.8 Hz, 1H), 4.55 (d, J=1.6 Hz, 1H), 3.82 (ddd, J=15.6, 10.8, 4.8 Hz, 1H), 3.75 (dd, J=3.2, 1.6 Hz, 1H), 3.65 (ddd, J=16.0, 10.4, 5.6 Hz, 1H), 3.59 (dd, J=9.2, 3.2 Hz, 1H), 3.52 (td, J=9.2, 2.8 Hz, 1H), 3.42 (dt, J=9.2, 8.8 Hz, 1H), 3.33 (s, 3H), 2.40 (dddd, J=13.6, 10.4, 5.6, 2.8 Hz, 1H), 2.09-1.81 (m, 1H).

### Preparation of methyl 4-(((3S,4S,5S,6R)-3,4,5-trihydroxy-6-((pyrimidin-2-ylamino)methyl)tetrahydro-2H-pyran-2-yl)oxy)benzoate (A10)

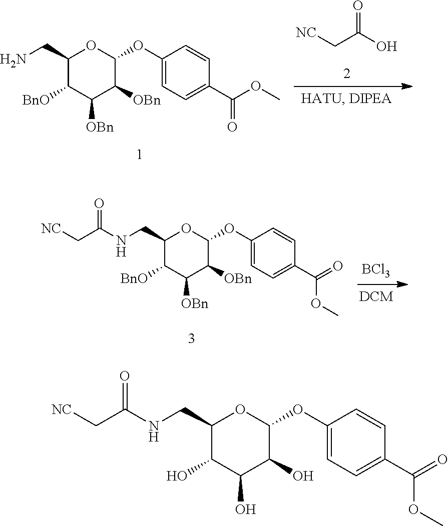

Step 1: A solution of 2 (1.00 eq), DIPEA (2.00 eq) and HATU (1.50 eq) in DMF (3 mL) was stirred at 25° C. for 10 min, then 1 (1.20 eq) was added. The mixture was stirred at 60° C. for 10 hours under N2 atmosphere. The mixture was concentrated and the purified by reverse phase (C18, 5-85% MeCN in water, 0.2% TFA) to afford 3 as colorless oil. LCMS found: [M+Na]⁺=673.

Step 2: To a solution of 3 (1.00 eq) in DCM (3 mL) was added BCl₃ (2.00 eq) at −40° C. The mixture was stirred at 25° C. for 10 min, then quenched with MeOH. The mixture was concentrated and then purified by reverse phase (C18, 5-40% MeCN in water, 0.3% TFA) to afford A10 as a white solid. LCMS found: [M+H]⁺=381. ¹H NMR (400 MHz, CD₃OD): δ 8.11-7.83 (m, 2H), 7.16 (d, J=8.8 Hz, 2ZH), 5.59 (d, J=1.6 Hz, 1H), 4.03 (dd, J=3.3, 1.8 Hz, 1H), 3.86 (d, J=6.8 Hz, 4H), 3.58 (d, J=3.1 Hz, 1H), 3.56 (d, J=3.6 Hz, 2H), 3.37 (d, J=30.3 Hz, 3H).

### Preparation of N-((2-((2R,3S,4S,5S,6S)-3,4,5,6-tetrahydroxytetrahydro-2H-pyran-2-yl)ethyl)sulfonyl)isoxazole-5-carboxamide (A11)

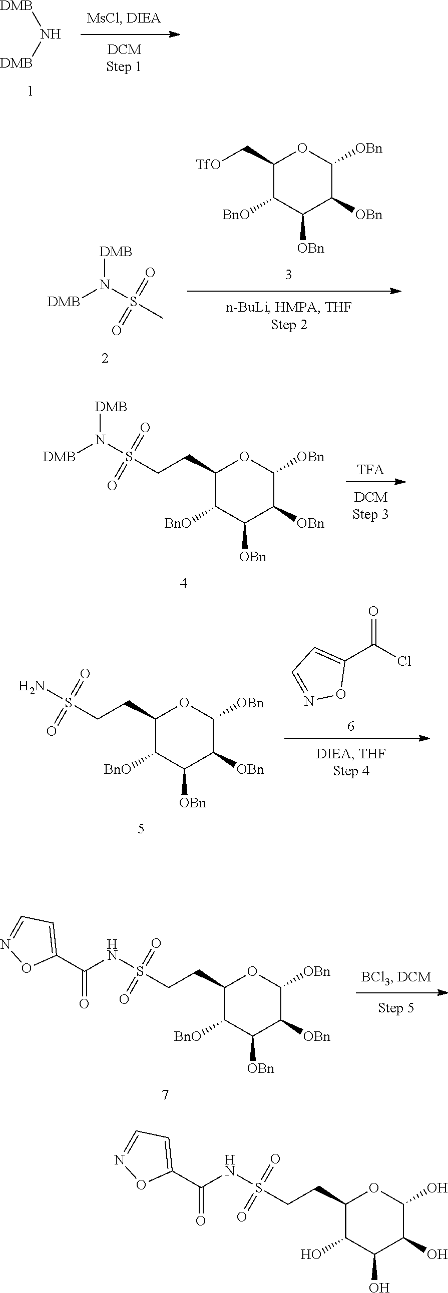

Step 1: To a solution of 1 (1.00 eq) in DCM (50 mL) was added DIEA (1.50 eq) and MsCl (1.50 eq) at 0° C. The reaction was stirred at 25° C. for 3 hours. TLC (PE/EA-2/1) showed reaction was completed. The reaction was extracted with EA and H₂O. The organic layer was concentrated and the residue was purified by silica gel chromatography (PE/EA=20/1 to 3/1) to give 2 as yellow oil. LCMS found: [M+H]⁺=332.

Step 2: To a solution of 2 (1.00 eq) in THF (20 mL) was added HMPA (1.50 eq) and n-BuLi (1.50 eq) at −78° C. The reaction was stirred for 2 hours, then 3 was added. The reaction was stirred at −78° C. for 1 hour, then quenched with NH₄Cl aq. The mixture was extracted with EA and H₂O. The organic layer was concentrated and the residue was purified by silica gel chromatography (PE/EA=10/1 to 3/1) to give 4 as yellow oil. LCMS found: [M+Na]⁺=940

Step 3: To a solution of 4 (1.00 eq) in DCM (10 mL) was added TFA (3 mL). The mixture was stirred at 25° C. for 18 hours. The reaction was concentrated and the residue was purified by silica gel chromatography (PE/EA=5/1 to 1/1) to give 5 as colorless oil. LCMS found: [M+H]⁺=618 Step 4: To a solution of 5 (1.00 eq) and DIPEA (3.00 eq) in DCM (10 mL) was added 6 (4.00 eq) at 0° C. The mixture was stirred at 25° C. for 18 hours. The mixture was extracted with EA and H₂O. The organic layer was concentrated and the residue was purified by silica chromatography (PE/EA=10/1 to 3/1) to give 7 as colorless oil. LCMS found: [M−H]+=711

Step 5: To a solution of 7 (1.00 eq) in DCM (10 mL) was added BCl₃ (10.0 eq) at −30° C. The reaction was stirred at 20° C. for 18 hours, then quenched with MeOH. The mixture was concentrated and purified by reverse phase (C18, 5-40% MeCN in water, 0.3% TFA) to afford A11 as colorless oil. LCMS found: [M−H]+=351. ¹H NMR (400 MHz, CD₃OD): δ 8.59 (d, J=1.6 Hz, 1H), 7.18 (d, J=1.6 Hz, 1H), 5.01 (d, J=1.6 Hz, 1H), 3.79-3.74 (m, 2H), 3.73-3.67 (m, 1H), 3.61-3.50 (m, 1H), 3.46-3.36 (m, 2H), 2.43-2.34 (m, 1H), 2.03-1.93 (m, 1H).

### Preparation of N-((2-((2R,3S,4S,5S,6S)-3,4,5,6-tetrahydroxytetrahydro-2H-pyran-2-yl)ethyl)sulfonyl)benzamide (A12)

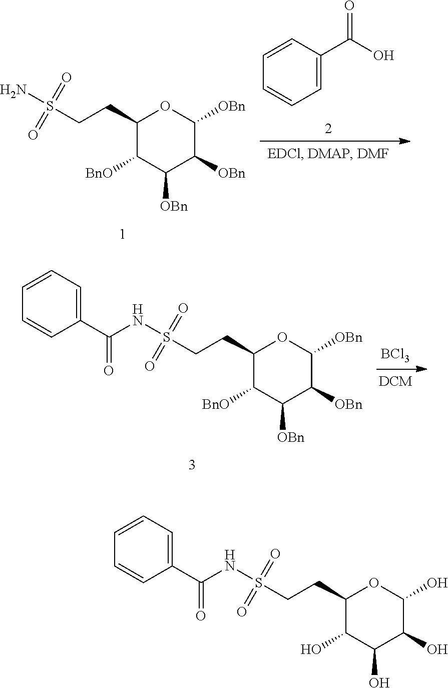

Step 1: To a solution of 1 (1.00 eq) in DMF (5 mL) was added EDCI (1.50 eq) and DMAP (1.50 eq) at 0° C. The reaction was stirred at 20° C. for 0.5 hour, then benzoic acid 2 (1.50 eq) was added. The mixture was stirred at 20° C. for 16 hours. The reaction was diluted with water, then extracted with EA, the organic layer was washed with brine, dried over anhydrous sodium sulfate, filtered and concentrated. The residue was purified by silica gel chromatography (PE/EA=10/1 to 3/1) to give 3 as white solid. LCMS found: [M+Na]⁺=744.

Step 2: To a solution of 3 (1.00 eq) in DCM (10 mL) was added BCl₃ (10.0 eq) at −20° C. The reaction was stirred at 20° C. for 18 hours, then quenched with MeOH. The reaction solution was concentrated, and the residue was purified by reverse phase (C18, 5-45% MeCN in water, 0.3% TFA) to afford A12 as yellow oil. LCMS found: [M−H]+=360. ¹H NMR (400 MHz, CD₃OD): δ 7.91 (d, J=8.0 Hz, 1H), 7.63-7.61 (m, 1H), 7.50 (t, J=8.0 Hz, 2H), 5.05 (s, 0.64H), 4.69 (s, 0.36H), 3.79-3.68 (m, 3H), 3.65-3.51 (m, 1H), 3.45-3.20 (m, 2H), 2.41-2.31 (m, 1H), 2.03-1.93 (m, 1H).

### Preparation of (2-((2R,3S,4R,5S)-3,4,5-trihydroxy-6-phenethyltetrahydro-2H-pyran-2-yl)ethyl)phosphonic acid (A13)

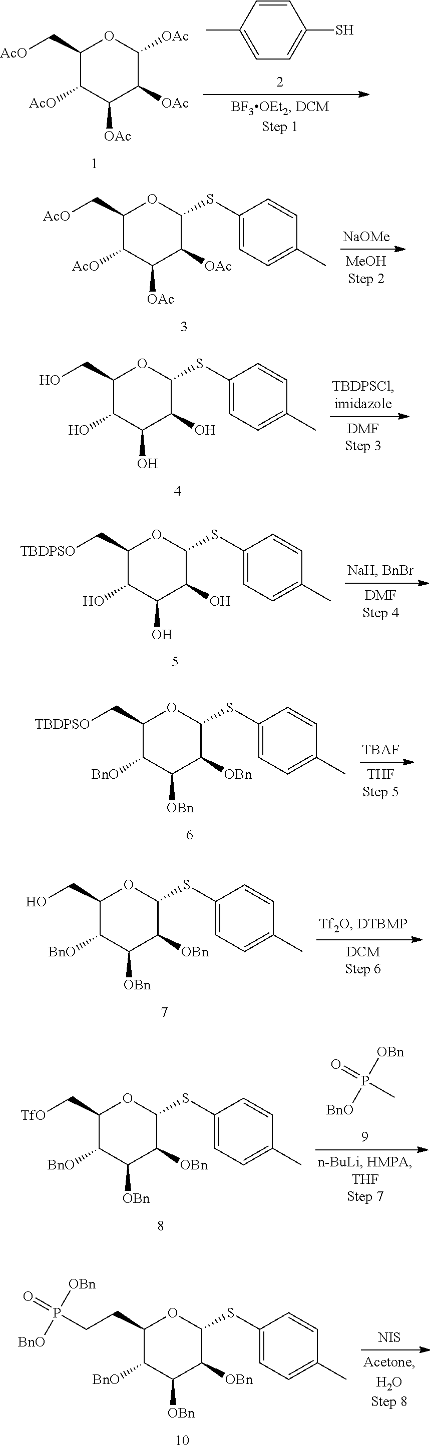

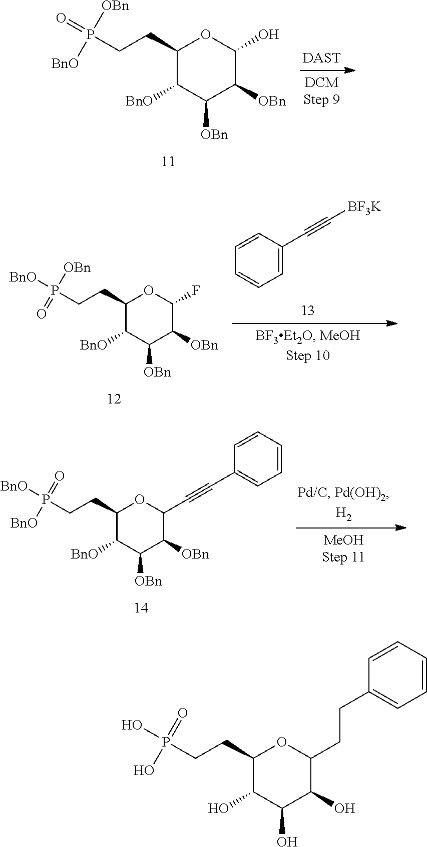

Step 1: To a solution of 1 (1.00 eq) in DCM (50 mL) was added 2 (1.50 eq) and BF₃·Et₂O(1.50 eq) at 25° C. The reaction was stirred at 25° C. for 16 hours. TLC (PE/EA=2/1) showed the reaction was completed. The reaction was quenched with NH₄HCO₃ aq, then the mixture was extracted with DCM. The organic layer was concentrated, and the residue was purified by silica gel chromatography (PE/EA=20/1 to 3/1) to give 3 as yellow oil. LCMS found: [M+Na]⁺=477.

Step 2: To a solution of 3 (1.00 eq) in MeOH (50 mL) was added NaOMe (0.10 eq) and stirred at 25° C. for 0.5 hour. TLC (PE/EA=2/1) showed the starting material was consumed completely. The pH of the reaction solution was adjusted to 6 with AmberliteI®120 H. The mixture was filtered through a Celite pad, and the filtrate was concentrated to give 4 as yellow solid. [M+Na]⁺=309.

Step 3: To a solution of 4 (1.00 eq) in DMF (20 mL) was added imidazole (1.50 eq) and TBDPSCl (1.50 eq) at 25° C. The reaction was stirred for 16 hours, then quenched with water. The mixture was extracted with EA. The organic layer was washed with brine, dried over anhydrous sodium sulfate, filtered and concentrated and the residue was purified by silica gel chromatography (PE/EA=5/1 to 1/1) to give 5 as yellow oil. LCMS found: [M+Na]⁺=547.

Step 4: To a solution of 5 (1.00 eq) in DMF (30 mL) was added NaH (4.00 eq). The mixture was stirred at 0° C. for 1 hour, then BnBr (4.00 eq) was added. The mixture was stirred 25° C. for 16 hours, then quenched with NH₄Cl aq. The mixture was extracted with EA. The organic layer was washed with brine, dried over anhydrous sodium sulfate, filtered and concentrated. The residue was purified by silica gel chromatography (PE/EA=20/1 to 5/1) to give 6 as colorless oil. LCMS found: [M+Na]⁺=817.

Step 5: To a solution of 6 (1.00 eq) in THF (10 mL) was added TBAF (1.00 eq) at 0° C. The mixture was stirred at 25° C. for 16 hours. The reaction was concentrated and purified by silica gel chromatography (PE/EA=5/1 to 2/1) to give 7 as colorless oil. LCMS found: [M+Na]⁺=579 Step 6: To a solution of 7 (1.00 eq) and 2,6-di-tert-butyl-4-methylpyridine (2.00 eq) in DCM was added Tf₂O (2.00 eq) dropwise at −30° C. The reaction was stirred at −30° C. for 0.5 hours, then quenched with saturated solution of sodium bicarbonate. The mixture was extracted with DCM. The organic layer was concentrated and the residue was purified by flash chromatography (silica gel, 0˜10% EA in PE) to give 8 as colorless oil. LCMS found: [M+Na]⁺=711.

Step 7: To a solution of 9 (2.00 eq) and HMPA (2.00 eq) in THF was added n-BuLi (2.00 eq) dropwise at −78° C. The reaction was stirred at −78° C. for 0.5 hour, then 8 (1.00 eq) was added at −78° C. over 1.5 hours. The mixture was stirred at 25° C. for 1 hour, then quenched with saturated solution of NH₄Cl. The solution was extracted with EA, the organic layer was concentrated and the crude product was purified by flash chromatography (silica gel, 0˜50% EA in PE) to give 10 as a colorless oil. LCMS found: [M+H]⁺=815. ¹H NMR (400 MHz, CDCl₃): δ 7.37-7.26 (m, 25H), 7.23 (d, J=8.1 Hz, 2H), 7.06 (d, J=8.0 Hz, 2H), 5.45 (d, J=1.4 Hz, 1H), 5.00-4.89 (m, 5H), 4.67 (q, J=12.4 Hz, 2H), 4.58 (d, J=12.1 Hz, 3H), 3.97-3.89 (m, 2H), 3.82 (dd,)=9.2, 3.0 Hz, 1H), 3.67 (t, J=9.3 Hz, 1H), 2.29 (s, 3H), 2.21 (dd, J=12.5, 7.7 Hz, 1H), 1.96-1.76 (m, 2H), 1.65-1.52 (m, 1H).

Step 8: To a solution of 10 (1.00 eq) in H₂O and Acetone was added NIS (1.50 eq) at 0° C. The mixture was stirred at 0° C. for 1.5 hours. The reaction was diluted with EA, washed with saturated solution of sodium bicarbonate and brine, dried over Na₂SO₄. The organic layer was separated and concentrated in vacuo. The crude product was purified by flash chromatography (silica gel, 0˜10% MeOH in DCM) to give 11 as a colorless oil. LCMS found: [M+H]⁺=709. ¹H NMR (400 MHz, CDCl₃): δ 7.36-7.29 (m, 16H), 7.26-7.18 (m, 9H), 5.17 (s, 1H), 5.00 (dd, J=7.4, 4.4 Hz, 2H), 4.96 (d, J=5.2 Hz, 1H), 4.93 (d, J=2.4 Hz, 1H), 4.90 (s, 1H), 4.76 (d, J=12.4 Hz, 1H), 4.71 (s, 1H), 4.61-4.56 (m, 3H), 4.01 (s, 1H), 3.95-3.88 (m, 2H), 3.82 (dd, J=6.3, 3.4 Hz, 1H), 3.64 (t, J=9.4 Hz, 1H), 2.21-2.14 (m, 1H), 1.95 (dt, J=18.4, 7.4 Hz, 2H), 1.85-1.76 (m, 1H).

Step 9: To a solution of 11 (1.00 eq) in THF was added DAST (1.30 eq) dropwise at −30° C. under N₂. The reaction was stirred at −30° C. for 0.5 hour, then quenched with methanol. The mixture was concentrated in vacuo. The crude product was purified by flash chromatography (silica gel, 0˜30% EA in PE) to give 12 as a colorless oil. LCMS found: [M+H]⁺=711. ¹H NMR (400 MHz, CDCl₃): δ 7.36-7.27 (m, 20H), 7.26-7.24 (m, 5H), 5.50-5.41 (m, 1H), 5.05-4.91 (m, 4H), 4.88 (d, J=10.8 Hz, 1H), 4.78 (d, J=12.2 Hz, 1H), 4.71-4.55 (m, 4H), 3.81 (dd, J=10.1, 3.8 Hz, 2H), 3.73-3.61 (m, 2H), 2.28-2.12 (m, 1H), 2.10-1.95 (m, 1H), 1.90-1.72 (m, 2H). ¹⁹F NMR (377 MHz, CDCl₃): 8-137.22 (s).

Step 10: To a solution of 12 (1.00 eq) and 13 (1.20 eq) in CH3CN was added BF₃·Et₂O(1.50 eq) dropwise at 0° C. The reaction was stirred at 0° C. for 20 min. The mixture was quenched with Et₃N, then concentrated in vacuo. The crude product was purified by flash chromatography (silica gel, 0˜30% EA in PE) to give 14 as a colorless oil. LCMS found: [M+H]⁺=793. ¹H NMR (400 MHz, CDCl₃): δ 7.42-7.26 (m, 30H), 5.06-4.89 (m, 6H), 4.74 (q, J=12.6 Hz, 2H), 4.65-4.55 (m, 3H), 4.03 (dd, J=9.2, 3.0 Hz, 1H), 3.89-3.86 (m, 1H), 3.75 (dd, J=11.8, 5.9 Hz, 1H), 3.66 (t, J=9.3 Hz, 1H), 2.27-2.05 (m, 2H), 1.93-1.74 (m, 2H). ³¹P NMR (162 MHz, CDCl₃): δ 33.65 (s).

Step 11: To a solution of 14 (1.00 eq) in MeOH was added Pd/C (10% purity), Pd(OH)₂ (10% purity) and AcOH (0.10 eq). The reaction was stirred at 50° C. under H₂ (15 Psi) for 12 hours. The mixture was filtered and concentrated in vacuo to give A13 as a yellow solid. LCMS found: [M+H]⁺=347. ¹H NMR (400 MHz, CD₃OD): δ 7.28-7.21 (m, 4H), 7.15 (ddd, J=6.2, 3.4, 1.6 Hz, 1H), 3.83-3.77 (m, 1H), 3.71-3.68 (m, 1H), 3.65 (dd, J=8.5, 3.4 Hz, 1H), 3.49 (t, J=8.5 Hz, 1H), 3.41 (td, J=8.7, 2.5 Hz, 1H), 2.78 (ddd, J=14.0, 9.3, 4.9 Hz, 1H), 2.62 (dt, J=13.7, 8.2 Hz, 1H), 2.19-2.00 (m, 3H), 1.77-1.67 (m, 3H). ³¹P NMR (162 MHz, CD₃OD): δ 28.22 (s).

### Preparation of 2-((2R,3S,4S,5S)-6-(but-3-yn-1-yloxy)-3,4,5-trihydroxytetrahydro-2H-pyran-2-yl)-N-(pyrimidin-2-yl)ethane-1-sulfonamide (A14)

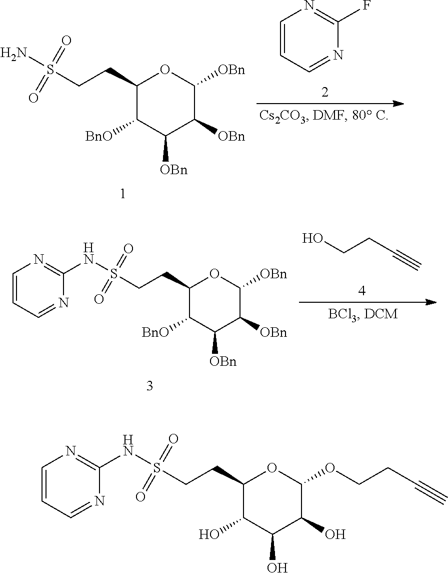

Step 1: To a solution of 1 (1.00 eq) in DMF (10 mL) was added Cs₂CO₃ (2.5 eq) and 2 (1.50 eq) at 25° C. The mixture was stirred at 80° C. for 3 hours. LCMS showed the starting material was consumed completely. The mixture was diluted with water, extracted with ethyl acetate, the combined organic layers were washed with saturated solution of NH₄Cl, dried over anhydrous sodium sulfate, filtered and concentrated under reduced pressure. The residue was purified by silica gel column chromatography (40-60% EA in PE) to afford 3 as a white solid. LCMS found: [M+H]⁺=696.

Step 2: To a solution of 3 (1.00 eq) in DCM (10 mL) was added BCl₃ (3.00 eq) at −30° C. The mixture was stirred at 25° C. for 3 hours. The mixture was quenched with 4 (20.0 eq) and stirred for 0.5 hour, then concentrated. The mixture was purified by prep-HPLC to afford A14 as a white solid. LCMS found: [M+H]⁺=388. ¹H NMR (400 MHz, CD₃OD): δ8.58 (d, J=4.9 Hz, 2H), 7.10 (t, J=4.9 Hz, 1H), 4.91-4.73 (m, 1H), 4.26-3.95 (m, 1H), 3.93-3.60 (m, 6H), 3.55 (dd, J=9.5, 6.4 Hz, 1H), 3.45 (t, J=9.5 Hz, 1H), 2.54-2.41 (m, 2H), 2.29-2.23 (m, 1H), 2.08-1.92 (m, 1H).

### Preparation of (2-((2R,3S,4S,5S)-3,4,5-trihydroxy-6-(2-hydroxy-9H-fluoren-3-yl)tetrahydro-2H-pyran-2-yl)ethyl)phosphonic acid (A15)

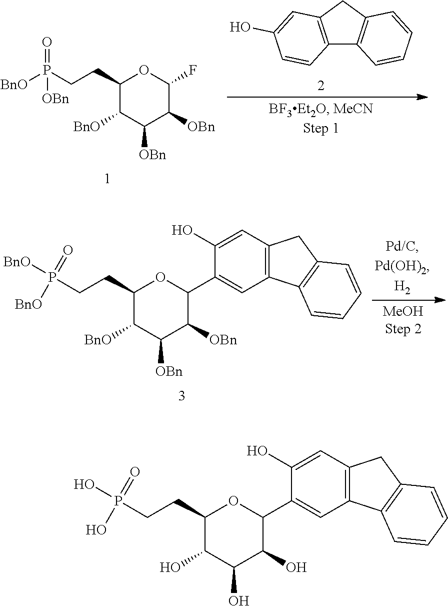 [SMILES: O=P(CC[C@H]1O[C@H](F)[C@@H](OCc2ccccc2)[C@@H](OCc2ccccc2)[C@@H]1OCc1ccccc1)(OCc1ccccc1)OCc1ccccc1 ; Oc1ccc2c(c1)Cc1ccccc1-2 ; O=P(CC[C@H]1OC(c2cc3c(cc2O)Cc2ccccc2-3)[C@@H](OCc2ccccc2)[C@@H](OCc2ccccc2)[C@@H]1OCc1ccccc1)(OCc1ccccc1)OCc1ccccc1 ; O=P(O)(O)CC[C@H]1OC(c2cc3c(cc2O)Cc2ccccc2-3)[C@@H](O)[C@@H](O)[C@@H]1O]

Step 1: To a solution of 1 (1.00 eq) and 2 (1.50 eq) in MeCN was added BF₃·Et₂O(2.00 eq) dropwised at 25° C. The reaction was stirred at 25° C. for 20 min. The resulting mixture was quenched with Et₃N and concentrated in vacuo. The crude product was purified by flash chromatography (silica gel, 0˜40% EtOAc in PE) to give 3 as a colorless oil. Chemical Formula: C₅₅H₅₃O₈P. LCMS found: [M+H]⁺=873.

Step 2: To a solution of 3 (1.00 eq) in MeOH was added Pd/C (10% purity) and Pd(OH)₂ (10% purity), the mixture was degassed and purged with H₂ several times, then stirred at 50° C. for 12 hr under H₂ (15 psi). The resulting mixture was filtered, the filtrate was concentrated in vacuo. The crude product was purified by prep-HPLC to give A15 as a white solid. Chemical Formula: C₂₀H₂₃O₈P. LCMS found: [M+H]⁺=423. ¹H NMR (400 MHz, CD₃OD): δ 7.84 (s, 1H), 7.67 (d, J=7.6 Hz, 1H), 7.45 (d, J=7.6 Hz, 1H), 7.28 (t, J=7.6 Hz, 1H), 7.16 (t, J=7.6 Hz, 1H), 6.97 (s, 1H), 4.89 (m, 0.5H), 4.83 (m, 0.5H), 4.17 (d, J=2.8 Hz, 1H), 3.78 (s, 2H), 3.67-3.62 (m, 1H), 3.58 (t, J=9.2 Hz, 1H), 3.33 (s, 1H), 2.35-2.24 (m, 1H), 2.11 (m, 1H), 1.95 (m, 1H), 1.86-1.74 (m, 1H).

### Preparation of (2-((2R,3S,4S,5S)-3,4,5-trihydroxy-6-(phenylethynyl)tetrahydro-2H-pyran-2-yl)ethyl)phosphonic acid (A16)

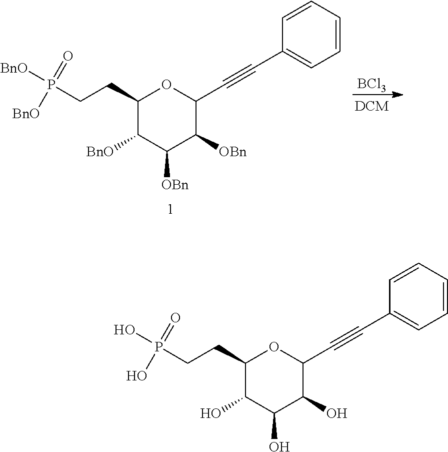

To a solution of 1 (1.00 eq) in DCM was added BCl₃ (10.00 eq) dropwise at −20° C. The mixture was stirred at 25° C. for 18 hr. The resulting mixture was quenched with methanol and concentrated in vacuo. The crude product was purified by prep-HPLC to give A16 as a white solid. Chemical Formula: C₁₅H₁₉O₇P. LCMS found: [M+H]⁺=343. ¹H NMR (400 MHz, CD₃OD): δ 7.47-7.42 (m, 2H), 7.38-7.32 (m, 3H), 4.81 (d, J=2.0 Hz, 1H), 3.99 (t, J=2.0 Hz, 1H), 3.89 (dd, J=9.2, 3.2 Hz, 1H), 3.77-3.69 (m, 1H), 3.51 (t, J=9.6 Hz, 1H), 2.27-2.12 (m, 1H), 2.03-1.88 (m, 1H), 1.86-1.73 (m, 1H), 1.72-1.57 (m, 1H).

### Preparation of (2-((2R,3S,4S,5S,6R)-3,4,5-trihydroxy-6-(1-phenethyl-1H-1,2,3-triazol-4-yl)tetrahydro-2H-pyran-2-yl)ethyl)phosphonic acid (A17)

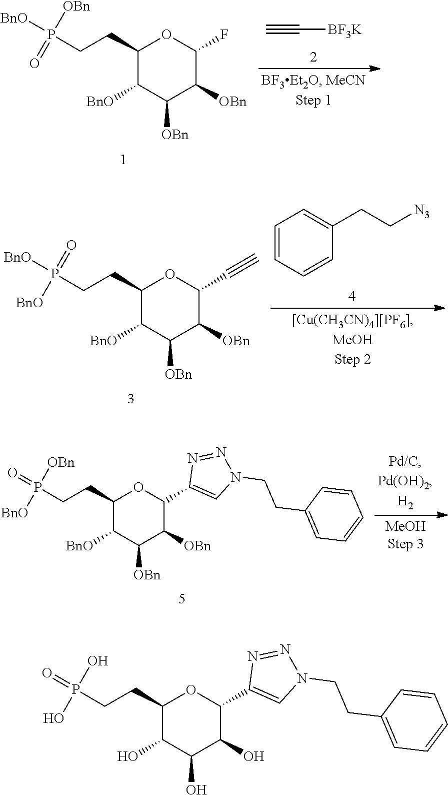

Step 1: To a solution of 1 (1.00 eq) and 2 (1.50 eq) in MeCN was added BF₃·Et₂O(2.00 eq) dropwise at 25° C. The mixture was stirred at 25° C. for 20 min. The resulting mixture was quenched with Et₃N and concentrated in vacuo. The crude product was purified by flash chromatography (silica gel, 0˜40% EtOAc in PE) to give 3 as a colorless oil. Chemical Formula: C₄₄H₄₅O₇P. LCMS found: [M+H]⁺=717. ¹H NMR (400 MHz, CDCl₃): δ 7.38-7.28 (m, 20H), 7.26 (s, 5H), 5.05-4.99 (m, 2H), 4.95 (ddd, J=11.8, 7.7, 2.1 Hz, 2H), 4.90 (d, J=10.7 Hz, 1H), 4.73-4.65 (m, 3H), 4.58 (d, J=10.9 Hz, 3H), 3.96 (dd, J=9.1, 3.0 Hz, 1H), 3.82-3.79 (m, 1H), 3.73-3.67 (m, 1H), 3.61 (t, J=9.3 Hz, 1H), 2.42 (d, J=2.3 Hz, 1H), 2.26-2.16 (m, 1H), 2.13-2.02 (m, 1H), 1.87-1.75 (m, 2H). ³¹P NMR (162 MHz, CDCl₃): δ 33.59 (s).

Step 2: To a solution of 3 (1.00 eq) and 4 (10.00 eq) in MeOH was added Cu(MeCN)₄PF₆ (2.00 eq) at 0° C. The reaction was stirred at 25° C. for 1 hr. The crude product was purified by flash chromatography (silica gel, 0˜50% EtOAc in PE) to give 5 as a colorless oil. Chemical Formula: C₅₂H₅₄N₃O₇P. LCMS found: [M+H]⁺=864.

Step 3: To a solution of 5 (1.00 eq) in MeOH was added Pd/C (10% purity) and Pd(OH)₂ (10% purity), the mixture was degassed and purged with H₂ several times, then stirred at 50° C. for 12 hr under H₂ (15 psi). The resulting mixture was filtered, the filtrate was concentrated in vacuo. The crude product was purified by prep-HPLC to give A17 as a white solid. Chemical Formula: C₁₇H₂₄N₃O₇P. LCMS found: [M+H]⁺=414. ¹H NMR (400 MHz, CD₃OD): δ 7.66 (s, 1H), 7.29-7.17 (m, 4H), 7.09 (d, J=7.1 Hz, 1H), 5.00 (s, 1H), 4.73-4.59 (m, 2H), 4.45 (s, 1H), 4.35-4.26 (m, 1H), 3.65-3.54 (m, 2H), 3.24-3.17 (m, 1H), 3.14-3.07 (m, 1H), 2.27-2.06 (m, 1H), 1.90-1.74 (m, 2H), 1.57-1.43 (m, 1H). ³¹P NMR (162 MHz, CD₃OD): δ 26.06 (s).

### Preparation of (2-((2R,3S,4S,5S,6R)-3,4,5-trihydroxy-6-((trimethylsilyl)ethynyl)tetrahydro-2H-pyran-2-yl)ethyl)phosphonic acid (A18)

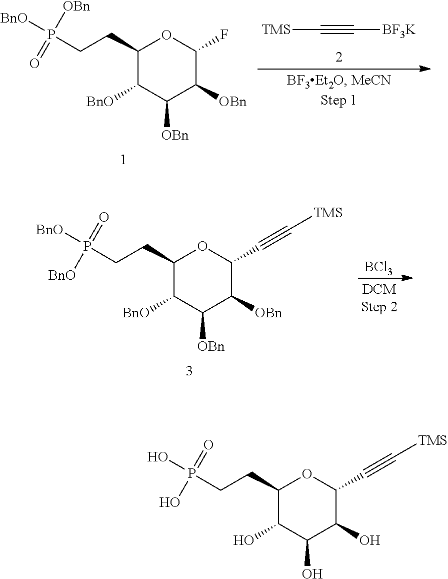

Step 1: To a solution of 1 (1.00 eq) and 2 (2.00 eq) in CH3CN was added BF₃·Et₂O(2.00 eq) dropwise at 0° C. The reaction was stirred at 25° C. for 30 min. The mixture was quenched with Et₃N and concentrated in vacuo. The crude product was purified by flash chromatography (silica gel, 0˜40% EtOAc in PE) to give 3 as a colorless oil. Chemical Formula: C₄₇H₅₃O₇PS. LCMS found: [M+H]⁺=789. ¹H NMR (400 MHz, CDCl₃): δ 7.34-7.22 (m, 20H), 7.21-7.18 (m, 5H), 4.99-4.83 (m, 5H), 4.68-4.59 (m, 3H), 4.56-4.49 (m, 3H), 3.87 (dd, J=8.8, 3.0 Hz, 1H), 3.73-3.70 (m, 1H), 3.63-3.53 (m, 2H), 2.17-1.98 (m, 2H), 1.81-1.62 (m, 2H), 0.06 (s, 9H). 31p NMR (162 MHz, CDCl₃): δ 33.70 (s).

Step 2: To a solution of 3 (1.00 eq) in DCM was added BCl₃ (10.00 eq) dropwise at −20° C. The reaction was stirred at 25° C. for 18 hr. The mixture was quenched with CH₃OH (0.5 mL) and concentrated in vacuo. The crude product was purified by prep-HPLC to give A18 as a white solid. Chemical Formula: Cl₂H₂₃O₇PSi. LCMS found: [M+H]⁺=339. ¹H NMR (400 MHz, CD₃OD): δ 4.59 (d, J=1.7 Hz, 1H), 3.91-3.86 (m, 1H), 3.79 (dd, J=9.2, 3.0 Hz, 1H), 3.63 (t, J=7.8 Hz, 1H), 3.44 (t, J=9.4 Hz, 1H), 2.23-2.09 (m, 1H), 2.07-1.89 (m, 1H), 1.83-1.58 (m, 2H), 0.18 (s, 9H). ³¹P NMR (162 MHz, CD₃OD): δ 30.28 (s).

### Preparation of (2-((2R,3S,4S,5S,6R)-6-((9H-fluoren-2-yl)oxy)-3,4,5-trihydroxytetrahydro-2H-pyran-2-yl)ethyl)phosphonic acid (A19)

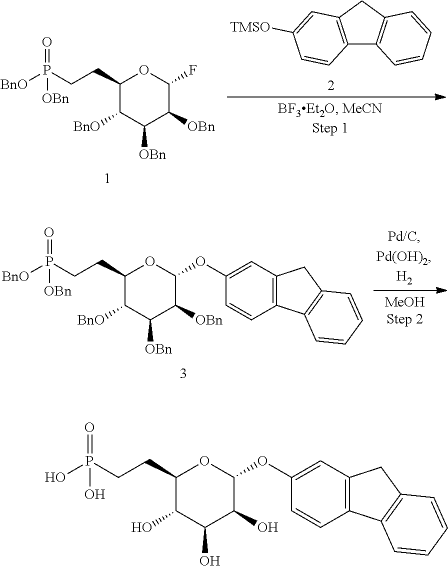

Step 1: To a solution of 1 (1.00 eq) and 2 (2.00 eq) in CH3CN was added BF₃·Et₂O(2.00 eq) dropwise at 0° C. The reaction was stirred at 25° C. for 30 min. The resulting mixture was quenched with Et₃N and concentrated in vacuo. The crude product was purified by flash chromatography (silica gel, 0˜50% EtOAc in PE) to give 3 as a colorless oil. Chemical Formula: C₅₅H₅₃O₈P. LCMS found: [M+H]⁺=873.

Step 2: A solution of 3 (1.00 eq) in MeOH was added Pd/C (10% purity) and Pd(OH)₂ (10% purity), the mixture was degassed and purged with hydrogen several times, then stirred at 50° C. for 12 hours under H₂ (15 Psi) atmosphere. The mixture was filtered, the filtrate was concentrated in vacuo. The crude product was purified by prep-HPLC to give A19 as a white solid. Chemical Formula: C₂₀H₂₃O₈P. LCMS found: [M+H]⁺=423. ¹H NMR (400 MHz, CD₃OD): δ 7.71 (d, J=8.2 Hz, 2H), 7.50 (d, J=7.6 Hz, 1H), 7.33-7.29 (m, 2H), 7.21 (t, J=7.4 Hz, 1H), 7.09 (dd, J=8.4, 2.2 Hz, 1H), 5.52 (d, J=1.4 Hz, 1H), 4.02 (dd, J=3.4, 1.7 Hz, 1H), 3.92-3.85 (m, 3H), 3.61-3.50 (m, 2H), 2.16-2.06 (m, 1H), 1.88-1.72 (m, 2H), 1.37-1.29 (m, 1H). ³¹P NMR (162 MHz, CD₃OD): δ 25.67 (s).

### Preparation of (2-((2R,3S,4S,5S,6R)-6-ethynyl-3,4,5-trihydroxytetrahydro-2H-pyran-2-yl)ethyl)phosphonic acid (A20)

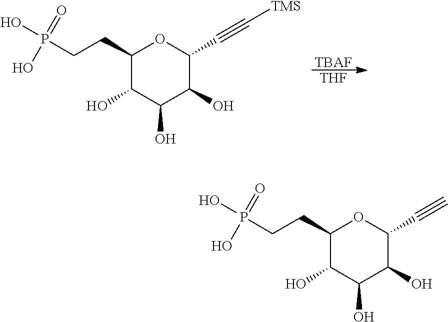

Step 1: To a solution of A26 (1.00 eq) in THF was added TBAF (1.30 eq) dropwise at 25° C. The reaction was stirred at 25° C. for 2 hr. The resulting mixture was concentrated in vacuo. The crude product was purified by prep-HPLC to give A20 as a white solid. Chemical Formula: C₉H₁₅O₇P. LCMS found: [M+H]⁺=267. ¹H NMR (400 MHz, CD₃OD): δ 4.61 (s, 1H), 3.91 (s, 1H), 3.81 (d, J=6.9 Hz, 1H), 3.69-3.59 (m, 1H), 3.49-3.39 (m, 1H), 3.07 (s, 1H), 2.24-2.08 (m, 1H), 2.03-1.87 (m, 1H), 1.82-1.58 (m, 2H). ³¹P NMR (162 MHz, CD₃OD): δ 29.91 (s).

### Preparation of (2-((2R,3S,4S,5S,6R)-3,4,5-trihydroxy-6-((E)-styryl)tetrahydro-2H-pyran-2-yl)ethyl)phosphonic acid (A21)

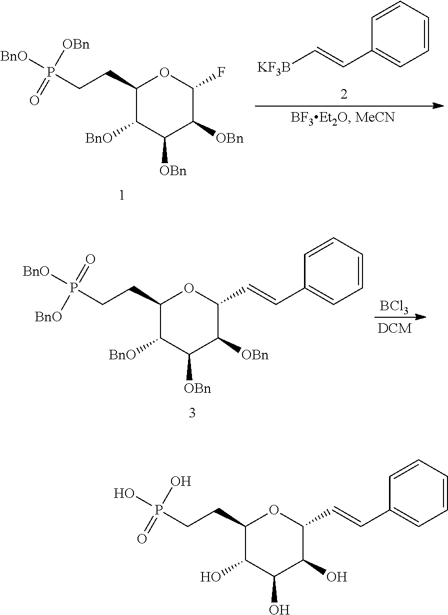

Step 1: To a solution of 1 (1.00 eq) and 2 (2.00 eq) in CH3CN (2 mL) was added BF₃·Et₂O(0.50 eq) dropwise at 25° C. The reaction was stirred at 25° C. for 0.5 hour. LCMS showed the desired mass was detected. The mixture was quenched with Et₃N (10.0 eq), then concentrated in vacuo. The crude product was purified by flash chromatography (silica gel, 0˜40% EtOAc in PE) to give 3 as a colorless oil. Yield: 35.8%. LC-MS (ESI) found: 795 [M+H]⁺.

Step 2: To a solution of 3 (1.00 eq) in DCM (3 mL) was added Boron trichloride (10.0 eq) dropwise at −20° C. The mixture was stirred at 25° C. for 2 hours. LCMS showed the desired mass was detected. The resulting mixture was quenched with MeOH (0.5 mL) and concentrated in vacuo. The crude product was purified by prep-HPLC to give A21 as a white solid. Yield: 10.0%. LCMS found: [M+H]⁺=345. ¹H NMR (400 MHz, CD₃OD): δ 7.44-7.40 (m, 2H), 7.32-7.27 (m, 2H), 7.24-7.19 (m, 1H), 6.65, 6.59 (d, J=16.0 Hz, 1H), 6.39, 6.27 (dd, J=16.0, 7.2 Hz, 1H), 4.46-4.33 (m, 1.5H), 4.29-4.23 (m, 1H), 4.02-3.93 (m, 1.5H), 3.86-3.81 (m, 0.5H), 3.69-3.62 (m, 0.5H), 2.15-1.95 (m, 2H), 1.85-1.66 (m, 2H).

### Preparation of (2-((2R,3S,4S,5S,6R)-3,4,5-trihydroxy-6-((7-hydroxy-9H-fluoren-2-yl)oxy)tetrahydro-2H-pyran-2-yl)ethyl)phosphonic acid (A22)

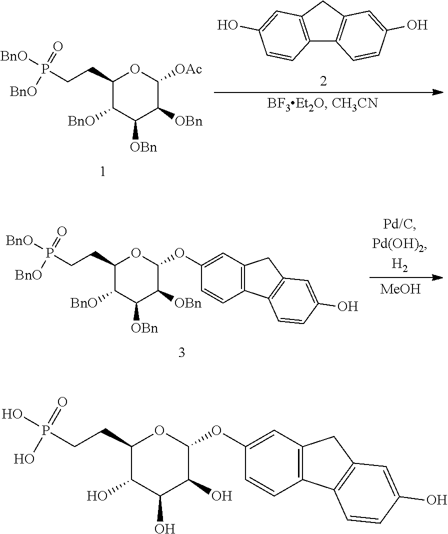 [SMILES: "MeOH" ; CC(=O)O[C@H]1O[C@H](CCP(=O)(OCc2ccccc2)OCc2ccccc2)[C@@H](OCc2ccccc2)[C@H](OCc2ccccc2)[C@@H]1OCc1ccccc1 ; Oc1ccc2c(c1)Cc1cc(O)ccc1-2 ; O=P(CC[C@H]1O[C@H](Oc2ccc3c(c2)Cc2cc(O)ccc2-3)[C@@H](OCc2ccccc2)[C@@H](OCc2ccccc2)[C@@H]1OCc1ccccc1)(OCc1ccccc1)OCc1ccccc1 ; O=P(O)(O)CC[C@H]1O[C@H](Oc2ccc3c(c2)Cc2cc(O)ccc2-3)[C@@H](O)[C@@H](O)[C@@H]1O]

Step 1: To a solution of 1 (1.00 eq) and 2 (2.00 eq) in CH3CN (5 mL) was added BF₃·Et₂O(0.50 eq) dropwise at 0° C. The reaction was stirred at 0° C. for 0.5 hour. The desired mass was observed by LCMS. The mixture was quenched with Et₃N (10.0 eq), then concentrated in vacuo. The crude product was purified by flash chromatography (silica gel, 10˜50% EtOAc in PE) to give 3 as a colorless oil. Yield: 47.9%. LC-MS (ESI) found: 911.5 [M+Na]⁺.

Step 2: To a solution of 3 (1.00 eq) in MeOH (5 mL) was added Pd/C (10% purity) and Pd(OH)₂ (10% purity), the mixture was degassed and purged with hydrogen for several times, then stirred at 50° C. for 12 hours under hydrogen (15 psi) atmosphere. The mixture was filtered, the filtrate was concentrated under reduced pressure, the residue was purified by prep-HPLC to afford A22 as a white solid. Yield: 71.6%. LCMS found: [M+H]⁺=439.3. ¹H NMR (400 MHz, CD₃OD): δ 7.55 (d, J=8.4 Hz, 1H), 7.50 (d, J=8.4 Hz, 1H), 7.22 (d, J=2.0 Hz, 1H), 7.02 (dd, J=8.4, 2.4 Hz, 1H), 6.95 (d, J=2.0 Hz, 1H), 6.77 (dd, J=8.4, 2.4 Hz, 1H), 5.50 (d, J=1.6 Hz, 1H), 4.03 (dd, J=3.6, 1.6 Hz, 1H), 3.88 (dd, J=9.2, 3.6 Hz, 1H), 3.77 (s, 2H), 3.58-3.50 (m, 2H), 2.20-2.07 (m, 1H), 1.96-1.80 (m, 1H), 1.81-1.65 (m, 1H), 1.48-1.34 (m, 1H).

### Preparation of N-hydroxy-N-(2-((2R,3S,4S,5S,6S)-3,4,5,6-tetrahydroxytetrahydro-2H-pyran-2-yl)ethyl)acetamide (A23)

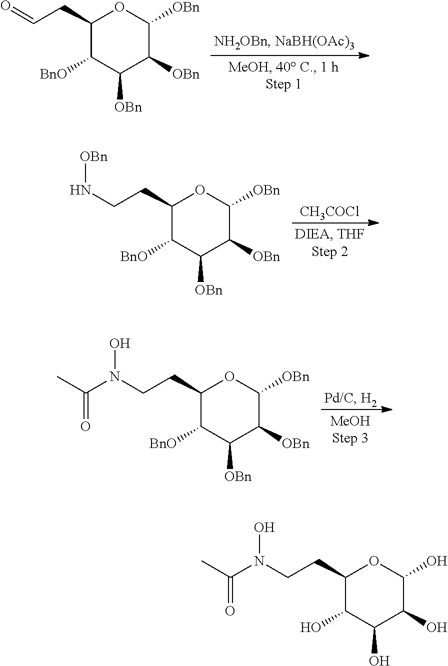

Step 1: To a mixture of N-(benzyloxy)-N-(2-((2R,3R,4S,5S,6S)-3,4,5,6-tetrakis(benzyloxy)tetrahydro-2H-pyran-2-yl)ethyl)acetamide (600 mg, 1.1 mmol) and benzylamine (174 mg, 1.65 mmol) in MeOH (5 mL) was added sodium triacetoxyborohydride (689 mg, 3.3 mmol) at rt, and the reaction mixture was stirred at 40 for 1 h under nitrogen. After completion, the mixture was concentrated and purified by flash (PE: EA=3:1) to give O-benzyl-N-(2-((2R,3R,4S,5S,6S)-3,4,5,6-tetrakis(benzyloxy)tetrahydro-2H-pyran-2-yl)ethyl) hydroxyl amine (400 mg, 55% yield) as colorless gel.

Step 2: To a mixture of O-benzyl-N-(2-((2R,3R,4S,5S,6S)-3,4,5,6-tetrakis(benzyloxy)tetrahydro-2H-pyran-2-yl)ethyl) hydroxylamine (400 mg, 0.6 mmol) in THF (5 mL) was added DIEA (220 mg, 1.8 mmol) and acetyl chloride (95 mg, 1.2 mmol) at rt, and the reaction mixture was stirred at rt for 1 h. After completion, the mixture was concentrated and purified by flash (PE: EA=5:1) to give N-(benzyloxy)-N-(2-((2R,3R,4S,5S,6S)-3,4,5,6-tetrakis(benzyloxy)tetrahydro-2H-pyran-2-yl)ethyl)acetamide (180 mg, 48% yield) as colorless gel.

Step 3: To a mixture of N-(benzyloxy)-N-(2-((2R,3R,4S,5S,6S)-3,4,5,6-tetrakis(benzyloxy)tetrahydro-2H-pyran-2-yl)ethyl)acetamide (180 mg, 0.25 mmol) in MeOH (5 mL) was added Pd/C (50 mg, 10% purity) at rt, and the reaction mixture was stirred at rt for 16 h under hydrogen atmosphere. After completion, the mixture was filtered, the filtrate was concentrated to give N-hydroxy-N-(2-((2R,3S,4S,5S,6S)-3,4,5,6-tetrahydroxytetrahydro-2H-pyran-2-yl)ethyl)acetamide (35 mg, 55% yield) as colorless gel. ¹H NMR (400 MHz, CD₃OD) δ 5.04 (s, 1H), 3.82-3.77 (m, 1H), 3.70 (ddd, J=8.9, 5.8, 3.1 Hz, 2H), 3.44 (t, J=9.5 Hz, 1H), 3.38-3.32 (m, 3H), 2.05 (ddd, J=10.2, 7.8, 4.1 Hz, 1H), 1.93 (s, 3H), 1.69-1.56 (m, 1H).

### Preparation of ((2R,3S,4S,5S,6R)-3,4,5-trihydroxy-6-phenoxytetrahydro-2H-pyran-2-yl)methyl sulfamate (A24)

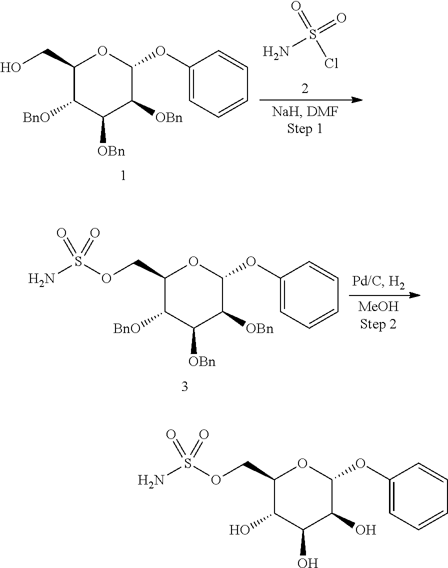

Step 1: To a solution of 1 (1.00 eq) in DMF was added NaH (2.00 eq, 60% purity) at 0° C. under N₂. The reaction was stirred at 0° C. for 0.5 hr. Then 2 (3.00 eq) was added. The reaction was stirred at 30° C. for 2.5 hr. The resulting mixture was quenched with saturated solution of ammonium chloride, extracted with EtOAc, the organic layer was washed with H₂O and brine, dried over Na₂SO₄, filtered and concentrated in vacuo. The crude product was purified by flash chromatography (silica gel, 10˜100% EA in PE) to give 3 as colorless oil. Chemical Formula: C₃₃H₃₅NO₈S. LCMS found: [M+H]⁺=606.

Step 2: To a solution of 3 (1.00 eq) in methanol was added Pd/C (10% purity), the mixture was stirred at 25° C. under H₂ (15 Psi) atmosphere for 12 h. The resulting mixture was filtered and the filtrate was concentrated in vacuo to give A24 as a white solid. Chemical Formula: Cl₂H₁₇NO₈S. LCMS found: [M+Na]⁺=358. ¹H NMR (400 MHz, CD₃OD): δ 7.33-7.25 (m, 2H), 7.10 (dd, J=8.6, 0.8 Hz, 2H), 7.01 (t, J=7.4 Hz, 1H), 5.45 (d, J=1.7 Hz, 1H), 4.35 (dd, J=10.8, 2.0 Hz, 1H), 4.29 (dd, J=10.8, 5.2 Hz, 1H), 4.02 (dd, J=3.3, 1.9 Hz, 1H), 3.91 (dd, J=9.1, 3.4 Hz, 1H), 3.84 (ddd, J=9.8, 5.1, 1.9 Hz, 1H), 3.80-3.74 (m, 1H).

### Preparation of methyl 4-(((3S,4S,5S,6R)-3,4,5-trihydroxy-6-((pyrimidin-2-ylamino)methyl)tetrahydro-2H-pyran-2-yl)oxy)benzoate (A25)

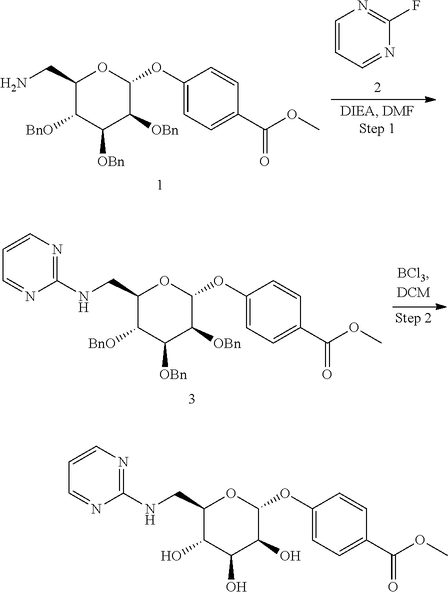 [SMILES: COC(=O)c1ccc(O[C@H]2O[C@H](CN)[C@@H](OCc3ccccc3)[C@H](OCc3ccccc3)[C@@H]2OCc2ccccc2)cc1 ; Fc1ncccn1 ; COC(=O)c1ccc(O[C@H]2O[C@H](CNc3ncccn3)[C@@H](OCc3ccccc3)[C@H](OCc3ccccc3)[C@@H]2OCc2ccccc2)cc1 ; COC(=O)c1ccc(O[C@H]2O[C@H](CNc3ncccn3)[C@@H](O)[C@H](O)[C@@H]2O)cc1]

Step 1: A solution of 1 (1.00 eq), 2 (1.50 eq) and DIPEA (2.00 eq) in DMF (2 mL) was stirred at 80° C. for 10 hours under nitrogen atmosphere. The mixture was diluted with water, extracted with EtOAc, the organic layer was dried over anhydrous sodium sulfate, filtered and concentrated under reduced pressure, the residue was purified by reverse phase (C18, 5-90% MeCN in water, 0.3% TFA) to afford 3 as colorless oil. LCMS found: [M+H]⁺=662.

Step 2: To a solution of 3 (1.00 eq) in DCM (3 mL) was added BCl₃ (2.00 eq) at −40° C. The mixture was stirred at rt for 10 min, then quenched with MeOH. The mixture was concentrated and then purified by reverse phase (C18, 5-50% MeCN in water, 0.3% TFA) to afford A25 as a white solid. Chemical Formula: C18H₂₁N₃O₇, LCMS found: [M+H]⁺=392. ¹H NMR (400 MHz, CD₃OD): δ 8.37 (s, 2H), 7.85 (dd, J=8.8, 1.6 Hz, 2H), 7.04 (d, J=8.8 Hz, 2H), 6.78 (d, J=5.2 Hz, 1H), 5.62 (s, 1H), 4.04 (dd, J=3.3, 1.8 Hz, 1H), 3.92-3.86 (m, 4H), 3.85-3.77 (m, 1H), 3.71 (dd, J=7.5, 2.4 Hz, 1H), 3.67 (d, J=9.1 Hz, 1H), 3.59 (dd, J=14.1, 7.6 Hz, 1H).

### Preparation of methyl 4-(((2R,3S,4S,5S,6R)-6-(((cyanomethyl)sulfonamido)methyl)-3,4,5-trihydroxytetrahydro-2H-pyran-2-yl)oxy)benzoate (A26)

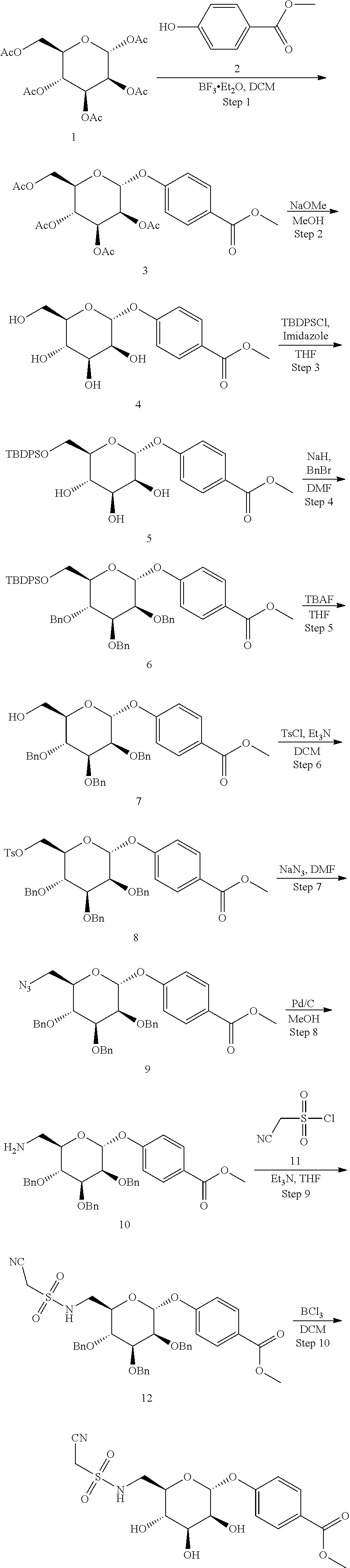

Step 1: To a solution of 1 (1.00 eq) and 2 (1.20 eq) in DCM (100 mL) was added Et₂O—BF₃ (2.00 eq) at 0° C. slowly. The reaction mixture was stirred at 25° C. for 16 hours. The reaction was diluted with water, extracted with ethyl acetate for three times. The combined organic layers were washed with saturated solution of sodium bicarbonate, dried over Na₂SO₄, filtered and concentrated under reduced pressure. The residue was purified by silica gel chromatography (10-40% ethyl acetate in petroleum ether) to afford 3 as a white solid. LCMS found: [M+Na]⁺=505.

Step 2: To a solution of 3 (1.00 eq) in MeOH (60 mL) was added NaOMe (0.1 eq), the mixture was stirred at 25° C. for 1 hour. The mixture was neutralized with (H) IR120, then filtered through a Celite pad, and the filtrate was concentrated to afford 4 as a colorless oil. LCMS found: [M+Na]⁺=337.

Step 3: To a solution of 4 (1.00 eq) in THF (40 mL) were added imidazole (2.00 eq) and TBDPSCl (1.50 eq) at 0° C. The mixture was stirred at 25° C. for 2 hours. The mixture was concentrated under reduced pressure. The residue was purified by silica gel chromatography (15-40% ethyl acetate in petroleum ether) to afford 5 as a colorless oil. LCMS found: [M+Na]⁺=575.

Step 4: To a solution of 5 (1.00 eq) in DMF (30 mL) was added BnBr (5.5 eq), followed by NaH (5.5 eq) slowly at 0° C. The resulting mixture was stirred at 25° C. for 2 hours, then quenched with water, extracted twice with ethyl acetate. The organic layer was washed with brine, dried over Na₂SO₄, filtered and concentrated. The residue was purified by silica gel column chromatography (5-20% ethyl acetate in petroleum ether) to afford 6 as a colorless oil.

Step 5: To a solution of 6 (1.00 eq) in THF (30 mL) was added TBAF (1.1 eq) at 0° C. The mixture was stirred at 25° C. for 16 hours. The mixture was concentrated under reduced pressure and the residue was purified by silica gel column chromatography (10-33% ethyl acetate in petroleum ether) to afford 7 as a yellow solid. LCMS found: [M+Na]⁺=607.

Step 6: To a solution of 7 (1.00 eq) in DCM (30 mL) were added TosCl (1.3 eq) and Et₃N (2.00 eq) at 0° C. The mixture was stirred at 25° C. for 16 hours, then concentrated under reduced pressure. The residue was purified by silica gel column chromatography (5-20% ethyl acetate in petroleum ether) to afford 8 as a colorless oil. LCMS found: [M+Na]⁺=761.

Step 7: To a solution of 8 (1.00 eq) in DMF (20 mL) was added NaN₃ (1.1 eq). The mixture was stirred at 50° C. for 16 hours, then diluted with water, extracted twice with ethyl acetate. The organic layer was washed with brine, dried over Na₂SO₄, filtered and concentrated. The residue was purified by silica gel column chromatography (5-20% ethyl acetate in petroleum ether) to afford 9 as a colorless oil. LCMS found: [M+Na]⁺=632.

Step 8: To a solution of 9 (1.00 eq) in MeOH (20 mL) was added Pd/C (10% purity) and NH₃.H₂O (0.05 mL). The mixture was degassed and purged with H₂ several times, then stirred at 25° C. for 1 hour under H₂ (15 psi) atmosphere. The mixture was filtered and the filtrate was concentrated under reduced pressure to afford 10 as a light yellow oil. LCMS found: [M+H]⁺=584.

Step 9: To a solution of 10 (1.00 eq) and Et₃N (2.00 eq) in THF (2.5 mL) was added 11 (1.20 eq) at 0° C. The reaction mixture was stirred at 25° C. for 2 hours. The mixture was concentrated under reduced pressure, the residue was purified by silica gel column chromatography (10-30% ethyl acetate in petroleum ether) to afford 12 as a pink solid. LCMS found: [M+Na]⁺=709.

Step 10: To a solution of 12 (1.00 eq) in DCM (3 mL) was added BCl₃ (5.00 eq) at −40° C. The reaction was stirred at −25° C. for 20 min. The mixture was quenched by MeOH slowly, then concentrated under reduced pressure, the residue was purified by reverse phase (C18, 5-50% MeCN in water, 0.3% TFA) to afford A26 as a colorless oil. LCMS found: [M+H]⁺=417. ¹H NMR (400 MHz, CD₃OD): δ 7.99 (d, J=8.9 Hz, 2H), 7.20 (d, J=8.9 Hz, 2H), 5.61 (d, J=1.6 Hz, 1H), 4.03 (dd, J=3.2, 1.9 Hz, 1H), 3.93-3.88 (m, 1H), 3.85 (s, 3H), 3.71 (t, J=9.6 Hz, 1H), 3.65-3.57 (m, 1H), 3.54 (dd, J=14.3, 2.4 Hz, 1H), 3.38 (dd, J=14.3, 6.1 Hz, 1H), 2.99 (s, 1H), 2.86 (s, 1H).

### Preparation of methyl 4-(((2R,3S,4S,5S,6R)-6-(((ethoxycarbonyl)amino)methyl)-3,4,5-trihydroxytetrahydro-2H-pyran-2-yl)oxy)benzoate (A27)

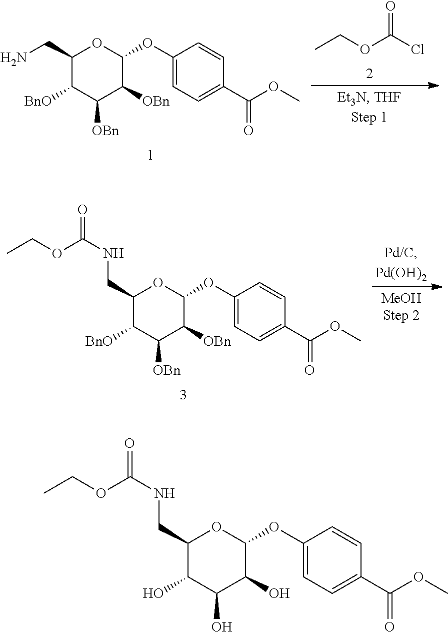

Step 1: To a solution of 1 (1.00 eq) and 2 (1.20 eq) in THF (2 mL) was added Et₃N (2.00 eq) at 0° C. The reaction mixture was stirred at 25° C. for 2 hours. The residue was concentrated under reduced pressure and then purified by silica gel column chromatography (5-20% ethyl acetate in petroleum ether) to afford 3 as a colorless oil. LCMS found: [M+Na]⁺=678.

Step 2: To a solution of 3 (1.00 eq) in MeOH (5 mL) were added Pd/C (10% purity) and Pd(OH)₂ (10% purity). The mixture was degassed and purged with H₂ several times, then stirred at 50° C. for 16 hours under H₂ (15 psi) atmosphere. The mixture was filtered, the filtrate was concentrated under reduced pressure, the residue was purified by the reverse phase (C18, 5-50% MeCN in water, 0.3% TFA) to afford A27 as a white solid. LCMS found: [M+H]⁺=386. ¹H NMR (400 MHz, CD₃OD): δ 7.96 (d, J=8.9 Hz, 2H), 7.17 (d, J=8.9 Hz, 2H), 5.56 (d, J=1.5 Hz, 1H), 4.02 (dd, J=3.4, 1.8 Hz, 1H), 3.98-3.82 (m, 6H), 3.63-3.44 (m, 3H), 3.21 (dd, J=14.1, 7.0 Hz, 1H), 1.13 (t, J=7.2 Hz, 3H).

### Preparation of dimethyl(methyl 4-{[(3S,4S,5S,6R)-6-[(benzoylamino)methyl]-3,4,5-trihydroxy-3,4,5,6-tetrahydro-2H-pyran-2-yl]oxy}benzoate (A28)

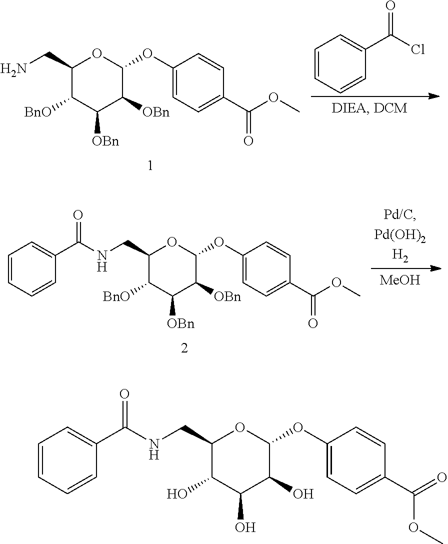

Step 1: To a solution of 1 (1.00 eq) and DIEA (2.00 eq) in DCM was added benzoyl chloride (1.20 eq) dropwise at 0° C., the mixture was stirred at 25° C. for 1 hour. The reaction system was quenched with water, then extracted with DCM, the organic layer was washed with saturated solution of sodium bicarbonate, dried over Na₂SO₄, filtered and concentrated under reduced pressure. The residue was purified by flash chromatography (5-40% ethyl acetate in petroleum ether) to give 2 as colorless oil. Chemical Formula: C₄₂H₄₁NO₈. LCMS found: [M+H]⁺=688.

Step 2: To a solution of 2 (1.00 eq) in MeOH was added Pd/C (10% purity) and Pd(OH)₂ (10% purity), then a drop of AcOH was added, the resulting mixture was degassed and purged with hydrogen several times, then stirred at 25° C. for 72 hours under H₂ (15 psi) atmosphere. The mixture was filtered, the filtrate was concentrated under reduced pressure, the residue was purified by prep-HPLC (0˜50% MeCN in H₂O) to give A28 as a grey solid. Chemical Formula: C21H23NO8. LCMS found: [M+H]⁺=418. ¹H NMR (400 MHz, MeOD) δ 7.78 (d, J=8.9 Hz, 2H), 7.59-7.55 (m, 2H), 7.49 (s, 1H), 7.36 (t, J=7.7 Hz, 2H), 7.12 (d, J=8.9 Hz, 2H), 5.61 (d, J=1.6 Hz, 1H), 4.58 (s, 1H), 4.04 (dd, J=3.4, 1.7 Hz, 1H), 3.88 (d, J=3.3 Hz, 1H), 3.85 (s, 3H), 3.69-3.60 (m, 2H), 3.42 (dd, J=13.8, 7.9 Hz, 1H).

### Preparation of methyl 4-(((3S,4S,5S,6R)-3,4,5-trihydroxy-6-(piperidin-1-ylmethyl)tetrahydro-2H-pyran-2-yl)oxy)benzoate (A29)

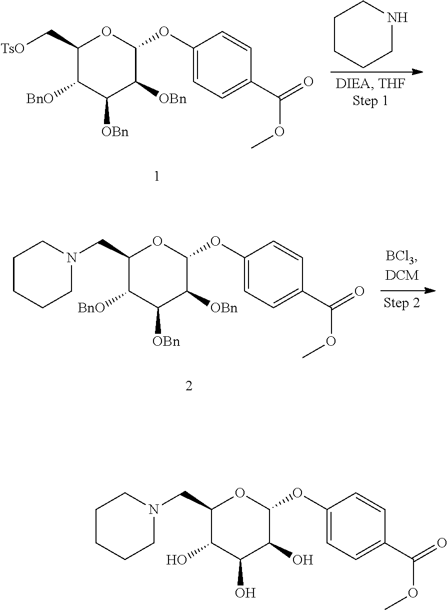

Step 1: To a solution of 1 (1.00 eq) in THF (1.00 mL) and DIEA (2.00 eq) were added piperidine (2.00 eq). The mixture was stirred at 70° C. for 2 h. The mixture was diluted with water, extracted with ethyl acetate, the organic layer was dried over anhydrous Na₂SO₄, filtered and concentrated under reduced pressure, the residue was purified by prep-TLC (DCM:MeOH=10:1) to afford 2 as a yellow oil. Chemical Formula: C₄₀H₄₅NO₇, LCMS found: [M+H]⁺=652.

Step 2: To a solution of 2 (1.00 eq) in DCM (11.00 mL) were added Boron trichloride (5.00 eq) at −30° C., the mixture was stirred at −30° C. for 1 h. The mixture was quenched with MeOH, then concentrated under reduced pressure, the residue was purified by prep-HPLC to afford A29 as a yellow solid. Chemical Formula: C₁₉H₂₇NO₇, LCMS found: [M+H]⁺=382. ¹H NMR (400 MHz, CD₃OD): δ8.10-7.97 (m, 2H), 7.34-7.09 (m, 2H), 5.79 (d, J=1.5 Hz, 1H), 4.13-4.07 (m, 1H), 3.97-3.85 (m, 4H), 3.62 (t, J=9.5 Hz, 1H), 3.45 (d, J=12.0 Hz, 1H), 3.39-3.33 (m, 2H), 3.30-3.25 (m, 1H), 2.95-2.80 (m, 2H), 2.72-2.59 (m, 1H), 1.86-1.65 (m, 3H), 1.56-1.26 (m, 3H).

### Preparation of methyl 4-(((2R,3S,4S,5S,6R)-3,4,5-trihydroxy-6-((oxetan-3-ylamino)methyl)tetrahydro-2H-pyran-2-yl)oxy)benzoate (A30)

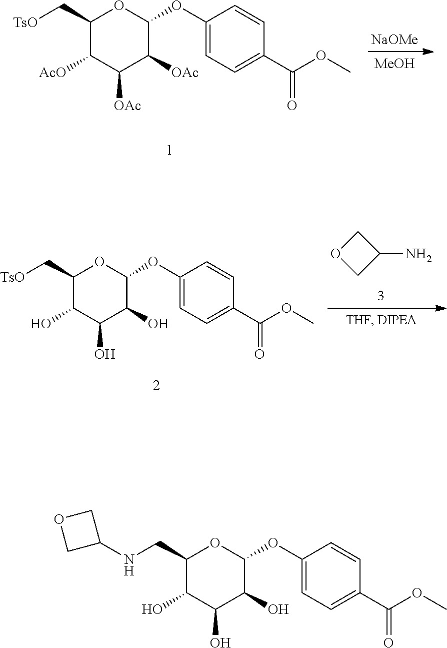 [SMILES: "MeOH" ; COC(=O)c1ccc(O[C@H]2O[C@H](COS(=O)(=O)c3ccc(C)cc3)[C@@H](OC(C)=O)[C@H](OC(C)=O)[C@@H]2OC(C)=O)cc1 ; COC(=O)c1ccc(O[C@H]2O[C@H](COS(=O)(=O)c3ccc(C)cc3)[C@@H](O)[C@H](O)[C@@H]2O)cc1 ; NC1COC1 ; "NaOMe" ; COC(=O)c1ccc(O[C@H]2O[C@H](CNC3COC3)[C@@H](O)[C@H](O)[C@@H]2O)cc1]

Step 1: To a solution of 1 (1.00 eq) in MeOH (3 mL) was added NaOMe (0.1 eq). The mixture was stirred at 0° C. for 1 hour under N₂ atmosphere. The mixture was adjusted to pH 7 with AmberliteIR120H resin. The mixture was filtered through a Celite pad, and the filtrate was concentrated to give 2 as a colorless oil. LCMS found: [M+Na]⁺=491.

Step 2: A solution of 2 (1.00 eq), 3 (3.00 eq) and DIPEA (2.00 eq) in THF (3 mL) was stirred at 100° C. for 16 hours in the sealed tube. The mixture was concentrated then purified by prep-HPLC to afford A30 as a white solid. LCMS found: [M+H]⁺=370. ¹H NMR (400 MHz, CD₃OD): δ 7.96 (d, J=8.8 Hz, 2H), 7.17 (d, J=8.8 Hz, 2H), 5.70-5.45 (m, 1H), 4.83-4.68 (m, 2H), 4.60-4.48 (m, 1H), 4.49-4.42 (m, 1H), 4.22-4.06 (m, 1H), 3.96-3.92 (m, 1H), 3.86 (s, 3H), 3.83-3.79 (m, 1H), 3.70 (d, J=7.6 Hz, 2H), 3.54-3.39 (m, 1H), 2.95-2.69 (m, 1H).

### Preparation of methyl 4-(((3S,4S,5S,6R)-3,4,5-trihydroxy-6-((4-methylpiperazin-1-yl)methyl)tetrahydro-2H-pyran-2-yl)oxy)benzoate (A31)

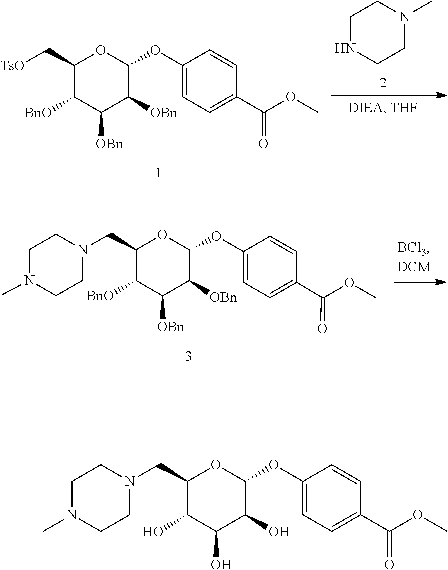

Step 1: A solution of 1 (1.00 eq), 2 (2.00 eq) and DIPEA (1.50 eq) in THF (10 mL) was stirred at 80° C. for 12 hours. The mixture was concentrated and the residue was was purified by silica gel column chromatography (1-5% PE in EA) to afford 3 as a yellow oil. LCMS found: [M+H]⁺=667. Step 2: To a solution of 3 (1.00 eq) in DCM (10.00 mL) was added BCl₃ (3.00 eq) at 0° C. for 2 h. LCMS showed the starting material was consumed completely and the major peak with desired mass was detected. The mixture was quenched with methanol, then concentrated under reduced pressure. The residue was purified by prep-HPLC to afford A31 as a white solid. LCMS found: [M+H]⁺=397. ¹H NMR (400 MHz, CD₃OD): δ8.03 (d, J=8.9 Hz, 2H), 7.20 (d, J=8.9 Hz, 2H), 5.66 (s, 1H), 4.07-4.04 (m, 1H), 3.91 (d, J=8.3 Hz, 1H), 3.88 (s, 3H), 3.76-3.58 (m, 2H), 3.32-3.25 (m, 2H), 3.04-2.30 (m, 11H).

### Preparation of methyl 4-(((3S,4S,5S,6R)-6-((cyclopropylamino)methyl)-3,4,5-trihydroxytetrahydro-2H-pyran-2-yl)oxy)benzoate (A32)

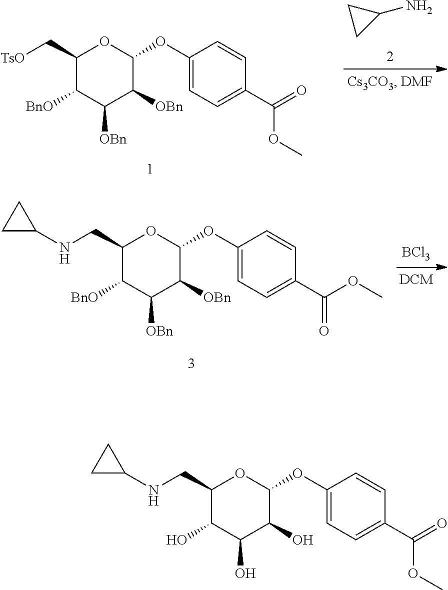

Step 1: A mixture of 1 (1.00 eq), 2 (2.00 eq) and Cs₂CO₃ (2.00 eq) in DMF was stirred at 80° C. for 16 hours. The mixture was diluted with water, then extracted with EA, the organic layer was washed with brine, dried over anhydrous sodium sulfate, filtered and concentrated. The residue was purified by flash chromatography (PE:EA=20:1 to 5:1) to afford 3 as a colorless solid. LCMS found: [M+H]⁺=624.

Step 2: To the solution of 3 (1.00 eq) in DCM was added BCl₃ (4.00 eq) at −30° C. and the mixture was stirred at −30° C. for 16 hours. The mixture was quenched with MeOH and concentrated. The crude product was purified by Prep-HPLC to afford A32 (formate) as a white solid. LCMS found: [M+H]⁺=354. ¹H NMR (400 MHz, CD₃OD): δ 8.44 (s, 1H), 8.02 (d, J=8.9 Hz, 2H), 7.22 (d, J=8.9 Hz, 2H), 5.70 (d, J=1.7 Hz, 1H), 4.08 (dd, J=3.2, 1.9 Hz, 1H), 3.93 (dd, J=9.4, 3.3 Hz, 1H), 3.90 (s, 3H), 3.83 (td, J=9.3, 2.8 Hz, 1H), 3.65 (t, J=9.6 Hz, 1H), 3.40 (dd, J=13.1, 2.9 Hz, 1H), 3.27-3.18 (m, 1H), 2.49-2.38 (m, 1H), 0.78-0.62 (m, 4H).

### Preparation of (2-((2R,3S,4S,5S,6S)-6-(but-3-yn-1-yloxy)-3,4,5-trihydroxytetrahydro-2H-pyran-2-yl)ethyl)(imino)(methyl)-16-sulfanone (A33)

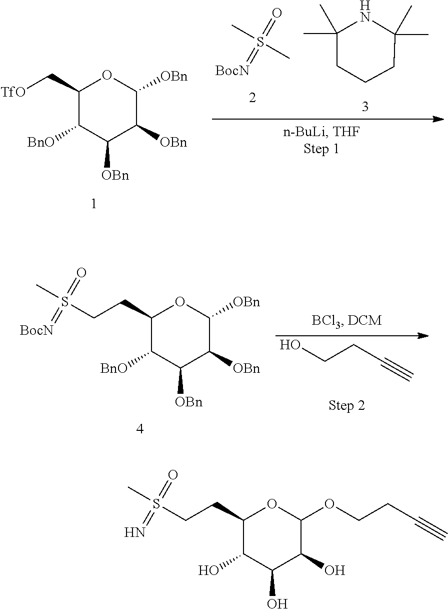

Step 1: To a solution of 3 (1.50 eq) in THF (10 mL) was added n-BuLi (1.70 eq) at 0° C. The mixture was stirred at 0° C. for 1 hour under N₂ atmosphere, then cooled to −78° C. 2 (1.20 eq) in THF (10 mL) was added slowly at −78° C. over 10 min. The mixture was stirred at −78° C. for 1 hour, then 1 (1.00 eq) in THF (10 mL) was added slowly at −78° C. over 30 min. The mixture was stirred at −78° C. for 1 hour, then quenched with NH₄Cl aq. The mixture was extracted with EA, the organic layer was dried over sodium sulfate, filtered and concentrated. The residue was purified by silica gel chromatography (PE/EA=20/1 to 3/1) to afford 4 as a colorless oil. LCMS found: [M+H]⁺=716.

Step 2: To a solution of 4 (1.00 eq) in DCM (3 mL) was added BCl₃ (10.0 eq) at −40° C. The mixture was stirred at 25° C. for 1 hour, then quenched with but-3-yn-1-ol (10.0 eq). The mixture was stirred at 25° C. for 1 hour, then concentrated. The residue was purified by prep-HPLC to afford A33 as a colorless oil. LCMS found: [M+H]⁺=308. ¹H NMR (400 MHz, CD₃OD): δ 4.93 (d, J=2.9 Hz, 0.5H), 4.76 (s, 0.5H), 4.32-4.21 (m, 0.5H), 4.07-3.98 (m, 0.5H), 3.86-3.79 (m, 1H), 3.79-3.71 (m, 1H), 3.66 (dd, J=9.4, 3.8 Hz, 1H), 3.55 (dt, J=9.6, 6.6 Hz, 1H), 3.51-3.33 (m, 2H), 3.25 (s, 1H), 3.03 (s, 3H), 2.52-2.34 (m, 3H), 2.28 (dd, J=9.0, 6.4 Hz, 1H), 2.07-1.91 (m, 1H).

### Preparation of tert-butyl(2-(2-(4-(2-(((2S,3S,4S,5S,6R)-3,4,5-trihydroxy-6-(2-(S-methylsulfonimidoyl)ethyl)tetrahydro-2H-pyran-2-yl)oxy)ethyl)-1H-1,2,3-triazol-1-yl)ethoxy)ethyl) carbamate (A34)

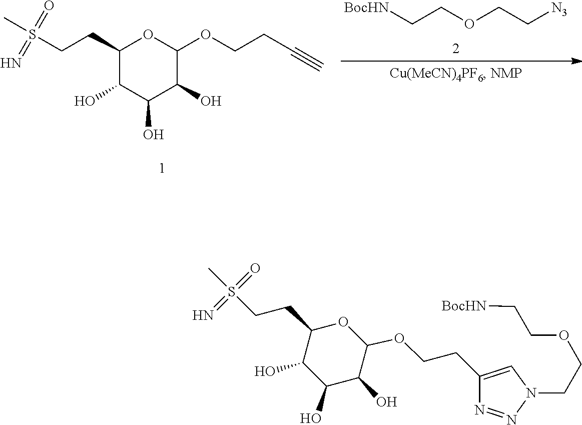 [SMILES: C#CCCOC1O[C@H](CCS(C)(=N)=O)[C@@H](O)[C@H](O)[C@@H]1O ; CC(C)(C)OC(=O)NCCOCCN=[N+]=[N-] ; CC(C)(C)OC(=O)NCCOCCn1cc(CCOC2O[C@H](CCS(C)(=N)=O)[C@@H](O)[C@H](O)[C@@H]2O)nn1]

Step 1: A solution of 1 (1.00 eq), 2 (1.10 eq) and Cu(MeCN)₄PF₆ (3.00 eq) in NMP was stirred at 25° C. under N₂ for 3 hours. The mixture was purified by reverse phase (C18 MeCN/0.1% TFA in H₂O) to afford A34 as colorless oil. LCMS found: [M+H]⁺=538. ¹H NMR (400 MHz, CD₃OD): δ 7.84 (d, J=3.8 Hz, 1H), 4.96-4.89 (m, 2H), 4.75 (s, 0.5H), 4.55 (dt, J=8.6, 4.2 Hz, 2H), 4.29 (s, 0.5H), 4.07-4.01 (m, 1H), 3.99-3.91 (m, 2H), 3.88-3.83 (m, 2H), 3.81-3.71 (m, 2H), 3.70-3.63 (m, 3H), 3.61-3.41 (m, 4H), 3.21 (dd, J=10.6, 5.5 Hz, 2H), 2.99 (q, J=6.7 Hz, 2H), 2.45-2.28 (m, 1H), 2.13-1.98 (m, 1H), 1.45 (s, 9H).

### Preparation of tert-butyl 11,18-dioxo-13,13-bis((3-oxo-3-((2-(2-(4-(2-(((2S,3S,4S,5S,6R)-3,4,5-trihydroxy-6-(2-(S-methylsulfonimidoyl)ethyl)tetrahydro-2H-pyran-2-yl)oxy)ethyl)-1H-1,2,3-triazol-1-yl)ethoxy)ethyl)amino) propoxy)methyl)-24-(4-(2-(((2S,3S,4S,5S,6R)-3,4,5-trihydroxy-6-(2-(S-methylsulfonimidoyl)ethyl)tetrahydro-2H-pyran-2-yl)oxy)ethyl)-1H-1,2,3-triazol-1-yl)-3,6,9,15,22-pentaoxa-12,19-diazatetracosanoate (A35)

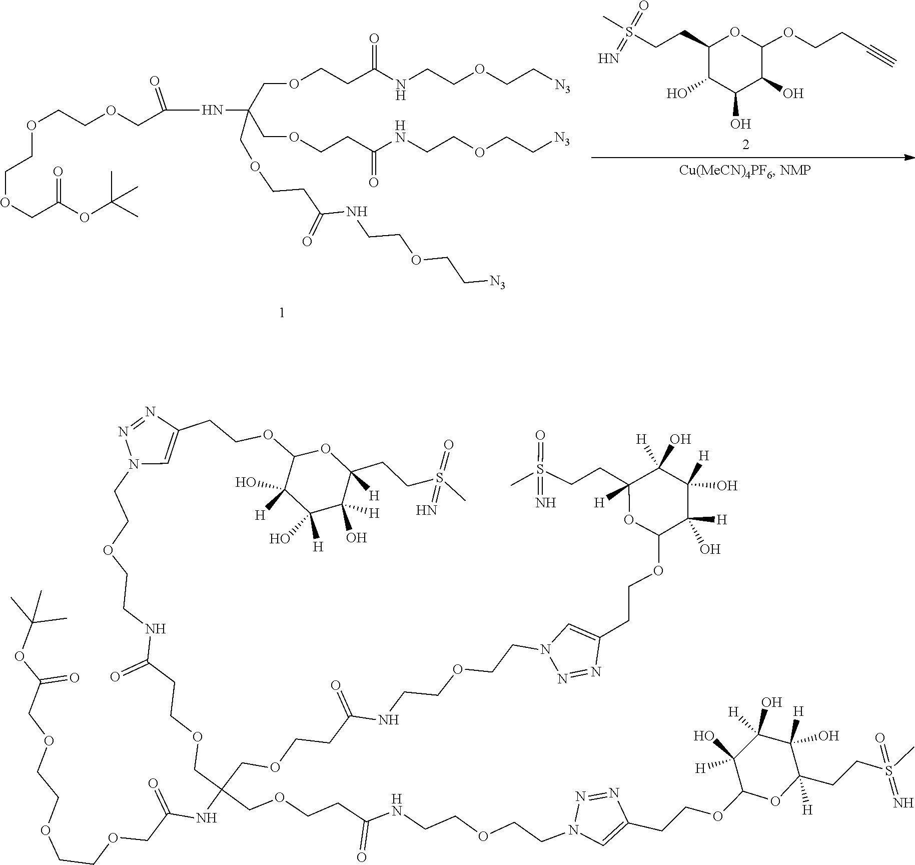 [SMILES: CC(C)(C)OC(=O)COCCOCCOCC(=O)NC(COCCC(=O)NCCOCCN=[N+]=[N-])(COCCC(=O)NCCOCCN=[N+]=[N-])COCCC(=O)NCCOCCN=[N+]=[N-] ; C#CCCOC1O[C@H](CCS(C)(=N)=O)[C@@H](O)[C@H](O)[C@@H]1O ; CC(C)(C)OC(=O)COCCOCCOCC(=O)NC(COCCC(=O)NCCOCCn1cc(CCOC2O[C@H](CCS(C)(=N)=O)[C@@H](O)[C@H](O)[C@@H]2O)nn1)(COCCC(=O)NCCOCCn1cc(CCOC2O[C@H](CCS(C)(=N)=O)[C@@H](O)[C@H](O)[C@@H]2O)nn1)COCCC(=O)NCCOCCn1cc(CCOC2O[C@H](CCS(C)(=N)=O)[C@@H](O)[C@H](O)[C@@H]2O)nn1]

Step 1: The solution of 1 (1.00 eq), 2 (3.30 eq) and Cu(MeCN)₄PF₆ (2.80 eq) in NMP was stirred at 25° C. under N₂ for 3 hours. The mixture was purified by reverse phase (C18 MeCN/0.1% TFA in H₂O) to afford A35 as colorless oil. LCMS found: [M+H]⁺=1856. ¹H NMR (400 MHz, CD₃OD): δ 7.84 (s, 3H), 4.93 (d, J=2.8 Hz, 2H), 4.75 (s, 1H), 4.58-4.52 (m, 6H), 4.27 (t, J=4.8 Hz, 1H), 4.06-3.99 (m, 6H), 3.98-3.91 (m, 6H), 3.90 (s, 2H), 3.86 (t, J=5.1 Hz, 6H), 3.79 (dd, J=3.2, 1.6 Hz, 2H), 3.76-3.72 (m, 2H), 3.71-3.64 (m, 32H), 3.59-3.43 (m, 12H), 3.38-3.32 (m, 7H), 3.04-2.93 (m, 6H), 2.57-2.49 (m, 1H), 2.43 (t, J=5.2 Hz, 6H), 2.35-2.27 (m, 1H), 2.19-1.99 (m, 4H), 1.47 (s, 9H).

### Preparation of (2-((2R,3S,4S,5S,6S)-6-(2-azidoethoxy)-3,4,5-trihydroxytetrahydro-2H-pyran-2-yl)ethyl)phosphonic acid (A36)

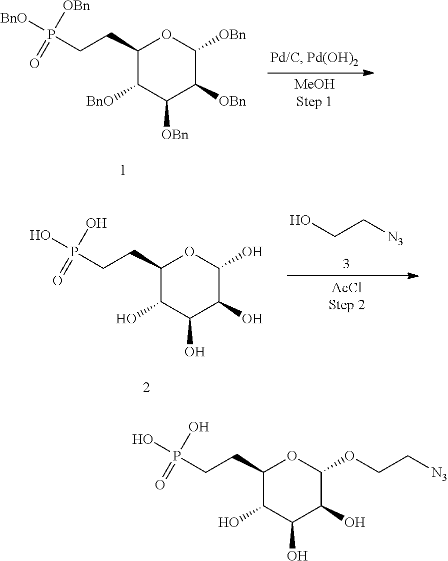 [SMILES: O=P(CC[C@H]1O[C@H](OCc2ccccc2)[C@@H](OCc2ccccc2)[C@@H](OCc2ccccc2)[C@@H]1OCc1ccccc1)(OCc1ccccc1)OCc1ccccc1 ; O=P(O)(O)CC[C@H]1O[C@H](O)[C@@H](O)[C@@H](O)[C@@H]1O ; [N-]=[N+]=NCCO ; [N-]=[N+]=NCCO[C@H]1O[C@H](CCP(=O)(O)O)[C@@H](O)[C@H](O)[C@@H]1O]

Step 1: To a solution of 1 (1.00 eq) in MeOH (5 mL) were added Pd/C (10% purity) and Pd(OH)₂ (10% purity), the mixture was degassed and purged with hydrogen several times, then stirred at 50° C. for 16 hours under hydrogen atmosphere (15 psi). The mixture was filtered through a Celite pad, and the filtrate was concentrated to give 2 as colorless oil. LCMS found: [M+H]⁺=259.

Step 2: To a solution of 2 (1.00 eq) in but-3-yn-1-ol (2 mL) was added AcCl (cat.), the mixture was stirred at 50° C. for 20 hours under nitrogen atmosphere. The mixture was concentrated under reduced pressure, and the residue was purified by reverse phase to give A36 as colorless oil. LCMS found: [M+H]⁺=328. ¹H NMR (400 MHz, CD₃OD): δ 4.77 (d, J=1.5 Hz, 1H), 3.85 (ddd, J=6.8, 5.0, 3.3 Hz, 2H), 3.73-3.68 (m, 1H), 3.67-3.58 (m, 1H), 3.52 (dd, J=11.7, 5.9 Hz, 1H), 3.50-3.43 (m, 1H), 3.39 (dd, J=16.5, 11.6 Hz, 2H), 2.27-2.09 (m, 1H), 2.07-1.92 (m, 1H), 1.82-1.69 (m, 2H).

### Preparation of tert-butyl(methyl(oxo) (2-((2R,3S,4S,5S,6S)-3,4,5,6-tetrahydroxytetrahydro-2H-pyran-2-yl)ethyl)-16-sulfanylidene) carbamate (A37)

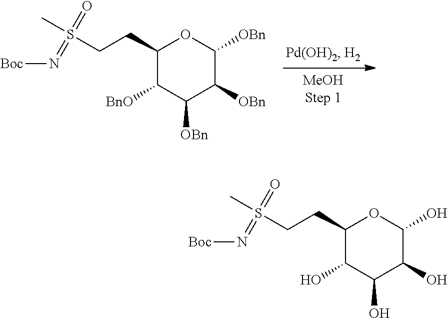 [SMILES: CC(C)(C)OC(=O)N=S(C)(=O)CC[C@H]1O[C@H](OCc2ccccc2)[C@@H](OCc2ccccc2)[C@@H](OCc2ccccc2)[C@@H]1OCc1ccccc1 ; CC(C)(C)OC(=O)N=S(C)(=O)CC[C@H]1O[C@H](O)[C@@H](O)[C@@H](O)[C@@H]1O]

Step 1: Under hydrogen atmosphere, a suspension of tert-butyl(methyl(oxo) (2-((2R,3R,4S,5S,6S)-3,4,5,6-tetrakis(benzyloxy)tetrahydro-2H-pyran-2-yl)ethyl)-16-sulfanylidene) carbamate (40 mg, 0.06 mmol) and Pd(OH)₂/C (10 mg, 10% purity) in MeOH (3 mL) was stirred at room temperature overnight. The reaction mixture was filtered and concentrated to give tert-butyl(methyl(oxo) (2-((2R,3S,4S,5S,6S)-3,4,5,6-tetrahydroxytetrahydro-2H-pyran-2-yl)ethyl)-16-sulfanylidene) carbamate (8.2 mg, 41% yield) as white solid. LC-MS (ESI) found: 356 [M+H]⁺. ¹H NMR (400 MHz, CD₃OD) δ 5.03 (d, J=1.4 Hz, 1H), 3.81-3.58 (m, 4H), 3.54-3.41 (m, 2H), 3.27 (S, 3H), 2.42-2.29 (m, 1H), 2.06-1.90 (m, 1H), 1.46 (s, 9H).

### Preparation of dimethyl(2-((2R,3S,4S,5S,6R)-3,4,5-trihydroxy-6-phenoxytetrahydro-2H-pyran-2-yl)ethyl)phosphine oxide (A38)

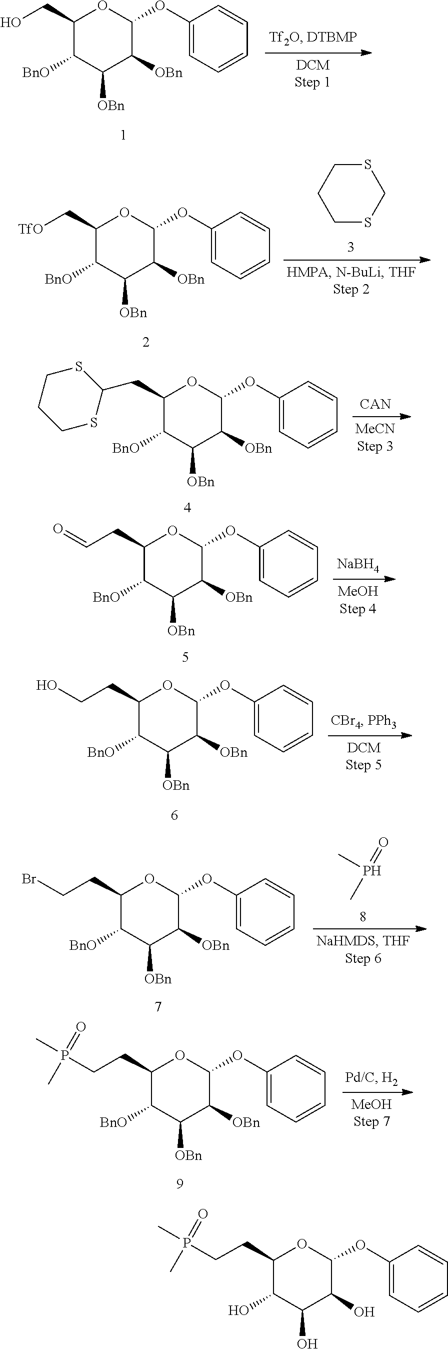

Step 1: To a solution of 1 (1.00 eq) and DTBMP (2.00 eq) in DCM was added Tf₂O (2.00 eq) dropwise at −20° C. The reaction was stirred at −20° C. for 0.5 h. The resulting mixture was quenched with saturated sodium bicarbonate solution, then extracted with DCM, the organic layer was washed with H₂O and brine, dried over Na₂SO₄ and concentrated in vacuo. The crude product was purified by flash chromatography (silica gel, 0˜10% EA in PE) to give 2 as colorless oil. Chemical Formula: C₃₄H₃₃F3O₈S.

Step 2: To a solution of 3 (2.00 eq) and HMPA (2.00 eq) in THF was added n-BuLi (2.00 eq) dropwise at −78° C. The reaction was stirred at −78° C. for 0.5 h. Then 2 (1.00 eq) in THF was added dropwise at −78° C., the resulting mixture was stirred at −78° C. for 1.5 h. The reaction was quenched with saturated solution of ammonium chloride, then extracted with EtOAc, the organic layer was washed with H₂O and brine, dried over Na₂SO₄ and concentrated in vacuo. The crude product was purified by flash chromatography (silica gel, 0˜10% EA in PE) to give 4 as colorless oil. Chemical Formula: C₃₇H₄₀O₅S2. LCMS found: [M+H]⁺=629.

Step 3: To a solution of 4 (1.00 eq) in MeCN was added CAN (1.20 eq) at 0° C. The reaction was stirred at 30° C. for 2 h. The mixture was diluted with H₂O, then extracted with EtOAc, the organic layer was washed with H₂O and brine, dried over Na₂SO₄ and concentrated in vacuo to afford 5 (crude) as a yellow oil. Chemical Formula: C₃₄H₃₄O₆. LCMS found: [M+H]⁺=539. Step 4: To a solution of 5 (1.00 eq) in MeOH was added NaBH₄ (3.00 eq) at 0° C., the mixture was stirred at 0° C. for 3 h. The resulting mixture was quenched with H₂O, then extracted with EtOAc, the organic layer was washed with H₂O and brine, dried over Na₂SO₄ and concentrated in vacuo.

The crude product was purified by flash chromatography (silica gel, 0˜10% EA in PE) to give 6 as a colorless oil. Chemical Formula: C₃₄H₃₆O₆. LCMS found: [M+H]⁺=541.

Step 5: To a solution of PPh₃ (1.50 eq) and CBr₄ (1.50 eq) in DCM was added 6 (1.00 eq) at 0° C., the mixture was stirred at 25° C. for 3 h. The resulting mixture was diluted with DCM, washed with H₂O and brine, dried over Na₂SO₄ and concentrated in vacuo. The crude product was purified by flash chromatography (silica gel, 0˜50% EA in PE) to give 7 as a colorless oil. Chemical Formula: C₃₄H₃₅BrO₅. LCMS found: [M+H]⁺=604. ¹H NMR (400 MHz, CDCl₃): δ 7.43-7.23 (m, 17H), 7.01 (t, J=7.4 Hz, 1H), 6.94 (t, J=6.8 Hz, 2H), 5.49 (d,J=1.8 Hz, 1H), 4.97 (d, J=10.9 Hz, 1H), 4.73 (ddd, J=30.7, 28.8, 11.7 Hz, 5H), 4.10 (dd, J=8.8, 3.0 Hz, 1H), 4.00-3.93 (m, 1H), 3.85-3.70 (m, 2H), 3.38 (td, J=9.2, 4.4 Hz, 1H), 3.21 (dd, J=17.7, 8.1 Hz, 1H), 2.39-2.25 (m, 1H), 2.17-1.98 (m, 1H).

Step 6: To a solution of 8 (1.00 eq) in THF was added NaHMDS (1.10 eq) dropwise at −78° C. The reaction was stirred at −78° C. for 0.5 h. Then 7 (0.13 eq) in THF was added dropwise at −78° C., the resulting mixture was stirred at −78° C. for 1.5 h. The reaction was quenched with saturated solution of ammonium chloride, then extracted with EtOAc, the organic layer was washed with H₂O and brine, dried over Na₂SO₄ and concentrated in vacuo. The crude product was purified by flash chromatography (silica gel, 0˜100% EA in PE) to give 9 as colorless oil. Chemical Formula: C₃₆H₄₁O₆P. LCMS found: [M+H]⁺=601.

Step 7: To a solution of 9 (1.00 eq) in MeOH was added Pd/C (10% purity), the mixture was degassed and purged with hydrogen several times, then stirred at 30° C. for 12 h under H₂ (15 Psi) atmosphere. The resulting mixture was filtered and the filtrate was concentrated in vacuo to give A38 as a colorless oil. Chemical Formula: C₁₅H₂₃O₆P. LCMS found: [M+H]⁺=331. ¹H NMR (400 MHz, CD₃OD): δ 7.33-7.25 (m, 2H), 7.11-6.97 (m, 3H), 5.51 (d, J=1.6 Hz, 1H), 4.01 (dd, J=3.2, 1.6 Hz, 1H), 3.87 (dd, J=8.8, 3.2 Hz, 1H), 3.53 (ddd, J=14.0, 12.0, 6.0 Hz, 2H), 2.13-2.03 (m, 1H), 1.95-1.84 (m, 1H), 1.72 (dtd, J=17.2, 8.8, 4.4 Hz, 1H), 1.62-1.53 (m, 1H), 1.45 (dd, J=13.2, 7.2 Hz, 6H). ³¹P NMR (162 MHz, CD₃OD): δ 50.83 (s).

### Preparation of imino(methyl) (((2S,3S,4S,5S,6R)-3,4,5-trihydroxy-6-phenoxytetrahydro-2H-pyran-2-yl)methyl)-16-sulfanone (A39)

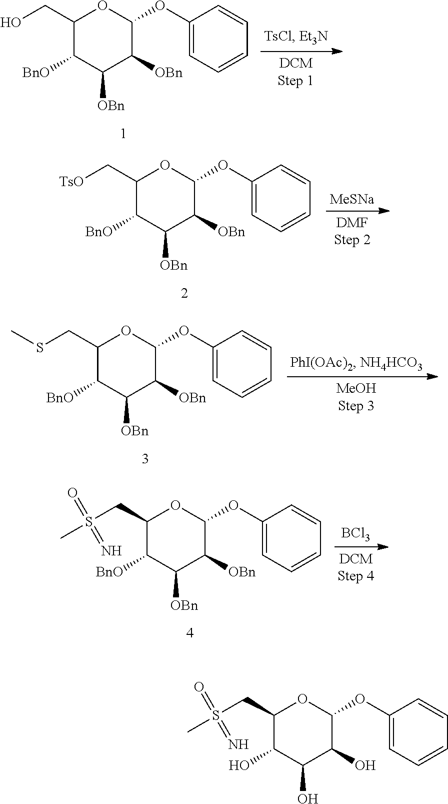

Step 1: The mixture of 1 (1.00 eq), TsCl (1.5 eq) and Et₃N (2.00 eq) in DCM was stirred at 25° C. for 12 h. The mixture was diluted with water, then extracted with ethyl acetate, the organic layer was dried over anhydrous sodium sulfate, filtered and concentrated. The residue was purified by flash chromatography (silica gel, PE: EA=20:1 to 5:1) to afford 2 as a colorless gel. Chemical Formula: C₄₀H₄₀O₈S, LCMS found: [M+H]⁺=681.

Step 2: The mixture of 2 (1.00 eq) and sodium methanethiolate (3.00 eq) in DMF was stirred at 60° C. for 12 h. The mixture was diluted with water, then extracted with ethyl acetate, the organic layer was dried over anhydrous sodium sulfate, filtered and concentrated. The residue was purified by flash chromatography (silica gel, PE: EA=50:1 to 10:1) to afford 3 as a colorless oil. Chemical Formula: C₃₄H₃₆O₅S. LCMS found: [M+H]⁺=557. ¹H NMR (400 MHz, CD₃OD) δ 7.48-7.22 (m, 17H), 7.09 (d, J=7.8 Hz, 2H), 7.02 (t, J=7.4 Hz, 1H), 5.55 (s, 1H), 4.94 (d, J=11.1 Hz, 1H), 4.82-4.75 (m, 2H), 4.73-4.58 (m, 3H), 4.12-4.07 (m, 2H), 3.94 (t, J=9.1 Hz, 1H), 3.88-3.80 (m, 1H), 2.82 (dd, J=14.1, 2.3 Hz, 1H), 2.63 (dd, J=14.0, 7.6 Hz, 1H), 2.01 (s, 3H).

Step 3: To a solution of 3 (1.00 eq) in MeOH was added NH₄HCO₃ (1.50 eq) and 1-[(acetoxyphenyl-23-iodanyl)oxy]ethan-1-one (2.30 eq), the mixture was stirred at 25° C. for 12 h. The mixture was concentrated and the residue was purified by flash chromatography (silica gel, DCM: MeOH=50:1 to 15:1) to afford 4 as a colorless gel. Chemical Formula: C₃₄H₃₇NO₆S. LCMS found: [M+H]⁺=588.

Step 4: To a solution of 4 (1.00 eq) in DCM was added BCl₃ (10.00 eq) at −30° C. and the mixture was stirred at 25° C. for 12 h. The mixture was quenched with MeOH and concentrated. The crude product was purified by prep-HPLC (FA condition) to afford A39 as a colorless oil. Chemical Formula: C₁₃H₁₉NO₆S. LCMS found: [M+H]⁺=318. ¹H NMR (400 MHz, CD₃OD) δ 7.36-7.25 (m, 2H), 7.13 (d, J=8.0 Hz, 1H), 7.09-6.95 (m, 2H), 5.58 (d, J=3.5 Hz, 1H), 4.45-4.30 (m, 1H), 4.19 (t, J=9.8 Hz, 1H), 4.12-4.05 (m, 1H), 3.96 (dd, J=9.3, 3.1 Hz, 1H), 3.67-3.51 (m, 2H), 3.13, 2.79 (d, J=7.6 Hz, 3H).

### Preparation of methyl(methylimino) (2-((2R,3S,4S,5S)-3,4,5,6-tetrahydroxytetrahydro-2H-pyran-2-yl)ethyl)-16-sulfanone (A40)

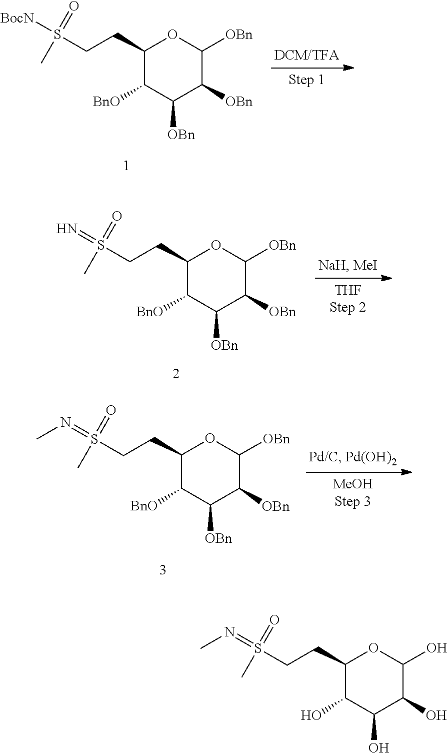

Step 1: To a solution of 1 (1.00 eq) in DCM (3 mL) was added TFA (1 mL). The mixture was stirred at 25° C. for 10 hours, then concentrated. The residue was purified by reverse phase (C18, 5-80% MeCN in water, 0.3% TFA) to afford 2 as colorless oil. LCMS found: [M+H]⁺=616.

Step 2: To a solution of 2 (1.00 eq) in THF (3 mL) was added NaH (60% purity, 1.50 eq) at 0° C. The mixture was stirred at 0° C. for 30 min, then Mel (1.20 eq) was added. The mixture was stirred at 25° C. for 5 hours, then quenched with saturated solution of NH₄Cl. The mixture was extracted with EA, the organic layer was dried over anhydrous sodium sulfate, filtered and concentrated. The residue was purified by reverse phase (C18, 5-85% MeCN in water, 0.3% TFA) to afford 3 as colorless oil. LCMS found: [M+H]⁺=630.

Step 3: To a solution of 3 (1.0 eq) in MeOH was added Pd/C (10% purity) and Pd(OH)₂ (10% purity) at 25° C., the mixture was degassed and purged with hydrogen several times, then stirred at 50° C. for 10 hours under hydrogen atmosphere (15 psi). The mixture was filtered and the filtrate was concentrated to give A40 as colorless oil. LCMS found: [M+H]⁺=270. ¹H NMR (400 MHz, CD₃OD): δ 5.09-4.99 (m, 1H), 4.09-4.00 (m, 2H), 3.86-3.79 (m, 2H), 3.76-3.72 (m, 3H), 3.70-3.55 (m, 1H), 3.48 (t, J=9.5 Hz, 1H), 2.97 (s, 3H), 2.48-2.32 (m, 1H), 2.16-2.05 (m, 1H).

### Preparation of (2S,3S,4S,5S,6R)-6-(aminomethyl)tetrahydro-2H-pyran-2,3,4,5-tetraol hydrochloride (A41)

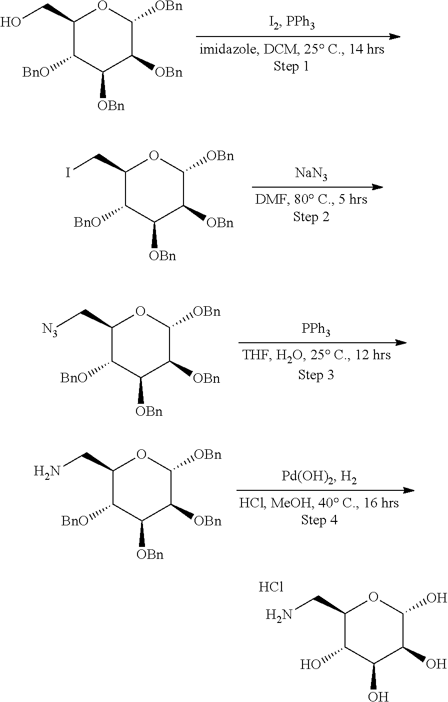

Step 1: To a solution of [(2R,3R,4S,5S,6S)-3,4,5,6-tetrabenzyloxytetrahydropyran-2-yl]methanol (17.8 g, 32.0 mmol) in THF (300 mL) was added PPh₃ (10.9 g, 41.7 mmol), imidazole (2.62 g, 38.4 mmol) and I₂ (40.7 g, 160 mmol). The mixture was stirred at 25° C. for 14 hrs. The mixture was quenched with a saturated Na₂S203 (200 mL) solution. The aqueous layer was extracted with ethyl acetate (500 mL). The organic layer was washed with brine (500 mL), dried over anhydrous Na₂SO₄, and concentrated in vacuo. The crude was 3 times diluted with cold diethyl ether, filtered and concentrated under reduced pressure. The residue was purified by column chromatography (SiO₂, petroleum ether:ethyl acetate=1:0 to 5:1) to afford (2S,3S,4S,5S,6S)-2,3,4,5-tetrabenzyloxy-6-(iodomethyl)tetrahydropyran (14.8 g, 69.0% yield) as yellow oil, LC-MS (ESI) found: 673 [M+Na]⁺.

¹H NMR (400 MHz, CDCl₃): δ 7.22-7.31 (m, 20H), 4.93 (t, J=10.8 Hz, 2H), 4.74 (t, J=11.6 Hz, 1H), 4.66 (t, J=6.40 Hz, 3H), 4.571 (s, 1H), 4.44 (d, J=12 Hz, 1H), 3.90-3.93 (m, 1H), 3.73-3.78 (m, 2H), 3.51-3.61 (m, 2H), 3.27-3.31 (m, 1H).

Step 2: To a solution of (2S,3S,4S,5S,6S)-2,3,4,5-tetrakis(benzyloxy)-6-(iodomethyl)tetrahydro-2H-pyran in DMF (10.0 mL) was added NaN₃ (1.48 g, 22.8 mmol). The mixture was stirred at 80° C. for 5 hrs. The mixture was diluted with H₂O (50.0 mL) and extracted with ethyl acetate (50.0 mL*3). The organic phases put together were washed with brine (50.0 mL), dried over anhydrous Na₂SO₄, filtered and concentrated under reduced pressure to afford (2R,3R,4S,5S,6S)-2-(azidomethyl)-3,4,5,6-tetrakis(benzyloxy)tetrahydro-2H-pyran (4.31 g, crude) as yellow oil.

Step 3: To a solution of (2R,3R,4S,5S,6S)-2-(azidomethyl)-3,4,5,6-tetrakis(benzyloxy)tetrahydro-2H-pyran (4.31 g, 7.62 mmol) in THF (10.0 mL) and H₂O (2.75 g, 152 mmol) was added PPh₃ (3.00 g, 11.4 mmol). The mixture was stirred at 25° C. for 12 hrs. The reaction mixture was filtered and purified by reversed-phase HPLC (0.10% NH₃·H₂O) to afford ((2R,3R,4S,5S,6S)-3,4,5,6-tetrakis(benzyloxy)tetrahydro-2H-pyran-2-yl)methanamine (3.00 g, 69.6% yield) as yellow oil.

LC-MS (ESI) found: 540 [M+H]⁺. ¹H NMR (400 MHz, CDCl₃): δ 7.18-7.42 (m, 20H), 4.85-5.02 (m, 2H), 4.37-4.68 (m, 8H), 3.99-4.15 (m, 1H), 3.71-3.91 (m, 4H).

Step 4: A solution of Pd(OH)₂/C (450 mg, 20% purity) in MeOH (5.00 mL) saturated with HCl (12 M, 66.3 μL) and ((2R,3R,4S,5S,6S)-3,4,5,6-tetrakis(benzyloxy)tetrahydro-2H-pyran-2-yl)methanamine (450 mg, 111.63 umol) (H₂) was stirred under 50 Psi at 40° C. for 16 hours in a 35 mL of sealed tube or autoclave. The reaction mixture was filtered and concentrated under reduced pressure to afford (2S,3S,4S,5S,6R)-6-(aminomethyl)tetrahydro-2H-pyran-2,3,4,5-tetraol hydrochloride (20 mg, 42.4% yield) as a black brown solid.

LC-MS (ESI) found: 180 [M+H]⁺. ¹H NMR (400 MHz, CDCl₃): δ 6.16-6.52 (m, 1H), 5.27-5.33 (m, 1H), 5.15-5.19 (m, 1H), 5.01-5.14 (m, 1H), 4.88-4.99 (m, 1H), 4.87-4.81 (m, 1H), 4.79-4.81 (m, 1H).

### Preparation of (2,6-difluorobenzyl) (2-((2R,3S,4S,5S,6R)-3,4,5-trihydroxy-6-phenoxytetrahydro-2H-pyran-2-yl)ethyl)phosphinic acid (A42)

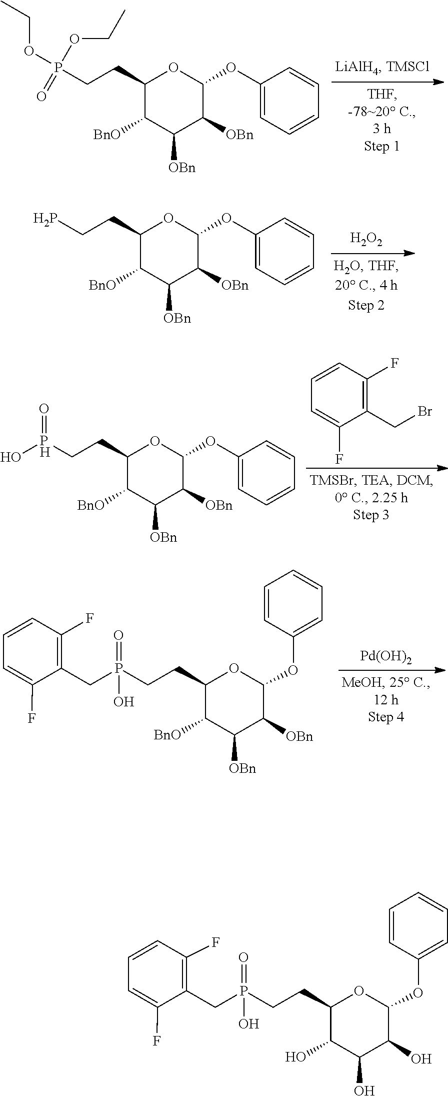

Step 1: To a solution of LAH (4.31 g, 113 mmol) in THF (30 mL) was degassed and purged with N₂ for 3 times, then TMSCl (9.87 g, 90.8 mmol, 11.5 mL) was added under −78° C. and the mixture was stirred at 20° C. for 2 hours under N₂ atmosphere. Then (2R,3R,4S,5S,6R)-3,4,5-tribenzyloxy-2-(2-diethoxyphosphorylethyl)-6-phenoxy-tetrahydropyran (5.00 g, 7.57 mmol) in THF (20 mL) was added under 0° C. The mixture was stirred at 20° C. for 1 h under N₂ atmosphere. LC-MS (ESI) found: 579 [M+Na]⁺. TLC (Petroleum ether: Ethyl acetate=10:1) showed (2R,3R,4S,5S,6R)-3,4,5-tribenzyloxy-2-(2-diethoxyphosphorylethyl)-6-phenoxy-tetrahydropyran was consumed completely. The reaction was quenched with H₂O (100 mL) at 0° C., then diluted with EtOAc (100 mL). After filtration, the mixture was extracted with EtOAc 300 mL (100 mL*3). The combined organic layers were washed with brine (100 mL), dried over anhydrous Na₂SO₄, filtered and concentrated under reduced pressure to give a residue. The residue was purified by column chromatography (SiO₂, Petroleum ether: Ethyl acetate=10:1) to afford 2-[(2R,3R,4S,5S,6R)-3,4,5-tribenzyloxy-6-phenoxy-tetrahydropyran-2-yl]ethylphosphane (3.2 g, 75.9% yield) as yellow oil. LC-MS (ESI) found: 579 [M+Na]⁺.

Step 2: To a solution of 2-[(2R,3R,4S,5S,6R)-3,4,5-tribenzyloxy-6-phenoxy-tetrahydropyran-2-yl]ethylphosphane (3.20 g, 5.75 mmol) in THF (10 mL) and H₂O (10 mL) was added H₂O₂ (5.21 g, 45.9 mmol, 4.42 mL, 30% purity). The mixture was stirred at 20° C. for 4 hours. LC-MS (ESI) found: 589 [M+H]⁺. The reaction mixture was quenched by addition saturated NaHSO₃ aqueous solution 20 mL at 0° C. and extracted with EtOAc (100 mL*3). The combined organic layers were washed with addition saturated NaHSO₃ aqueous solution (50 mL*3), dried over anhydrous Na₂SO₄, filtered and concentrated under reduced pressure to give a residue. The residue was purified by prep-HPLC (YMC Triart C18 70*250 mm*7 um; mobile phase: [water (TFA)-ACN]; B %: 42%-72%, 23 min) to afford 2-[(2R,3R,4S,5S,6R)-3,4,5-tribenzyloxy-6-phenoxy-tetrahydropyran-2-yl]ethylphosphinic acid (2.50 g, 73.8% yield) as yellow oil. LC-MS (ESI) found: 589 [M+H]⁺.

Step 3: A mixture of 2-[(2R,3R,4S,5S,6R)-3,4,5-tribenzyloxy-6-phenoxy-tetrahydropyran-2-yl]ethylphosphinic acid (200 mg, 339 umol), TMSBr (156 mg, 1.02 mmol) and TEA (103 mg, 1.02 mmol) in DCM (4 mL) was degassed and purged with N₂ for 3 times. Then the mixture was stirred at 0° C. for 15 min under N₂ atmosphere. Then 2-(bromomethyl)-1,3-difluoro-benzene (70.3 mg, 339 μmol) was added under 0° C. Then the mixture was stirred at 0° C. for 2 hours under N₂ atmosphere. LC-MS (ESI) found: 715 [M+H]⁺. The residue was purified by prep-HPLC (Welch Xtimate C18 150*25 mm*5 um; mobile phase: [water (TFA)−ACN]; B %: 60%-90%, 10 min) to afford (2,6-difluorophenyl)methyl-[2-[(2R,3R,4S,5S,6R)-3,4,5-tribenzyloxy-6-phenoxy-tetrahydropyran-2-yl]ethyl]phosphinic acid (20 mg, 8.24% yield) as colorless oil. ¹H NMR (400 MHz, MeOD): δ 7.35-6.90 (m, 20H), 6.74 (t, J=7.6 Hz, 3H), 5.45 (d, J=1.1 Hz, 1H), 5.00-4.50 (m, 6H), 4.04 (dd, J=2.9, 9.1 Hz, 1H), 3.91 (s, 1H), 3.70 (t, 1H, J=9.3 Hz), 3.60-3.50 (m, 1H), 3.01 (d, 2H, J=16.6 Hz), 2.20-2.00 (m, 1H), 1.90-1.70 (m, 2H), 1.50-1.30 (m, 1H) and LC-MS (ESI) found: 715 [M+H]⁺.

Step 4: A mixture of (2,6-difluorophenyl)methyl-[2-[(2R,3R,4S,5S,6R)-3,4,5-tribenzyloxy-6-phenoxy-tetrahydropyran-2-yl[ethyl]phosphinic acid (20 mg, 27.9 μmol) and Pd(OH)₂/C (19.6 mg, 20% purity) in MeOH (3 mL) was degassed and purged with H₂ (15 Psi) for 3 times, and then the mixture was stirred at 25° C. for 2 hours under H₂ (15 Psi) atmosphere. LC-MS (ESI) found: 445 [M+H]⁺. The residue was purified by prep-HPLC (Welch Xtimate C18 150*25 mm*5 μm; mobile phase: [water (TFA)−ACN]; B %: 15%-45%, 10 min) to afford (2,6-difluorophenyl)methyl-[2-[(2R,3S,4S,5S,6R)-3,4,5-trihydroxy-6-phenoxy-tetrahydropyran-2-yl]ethyl]phosphinic acid (4 mg, 30.8% yield) as a white solid. ¹H NMR (400 MHz, MeOD): δ 7.40-7.20 (m, 3H), 7.10-6.90 (m, 5H), 5.47 (d, J=1.5 Hz, 1H), 4.01 (dd, J=1.8, 3.3 Hz, 1H), 3.86 (dd, J=3.4, 9.1 Hz, 1H), 3.60-3.40 (m, 2H), 3.16 (d, J=16.8 Hz, 2H), 2.20-2.10 (m, 1H), 2.00-1.60 (m, 2H), 1.43 (d, J=11.0 Hz, 1H) and LC-MS (ESI) found: 445 [M+H]⁺.

### Preparation of methyl((2S)-2-((2R,3S,4S,5S,6R)-3,4,5-trihydroxy-6-phenoxytetrahydro-2H-pyran-2-yl)cyclopropyl)phosphonic acid (A43)

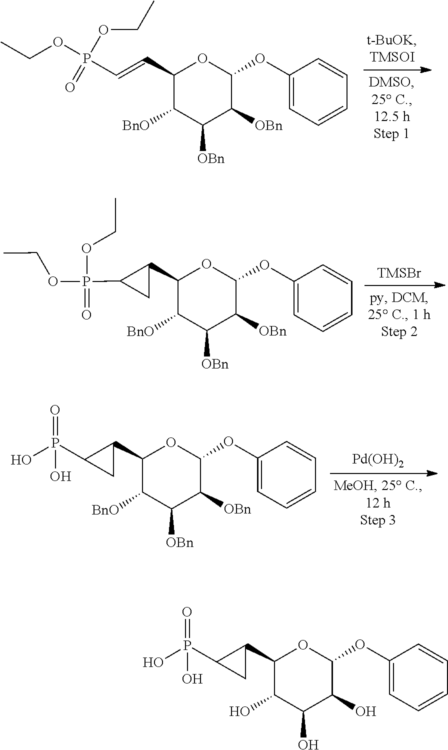

Step 1: To a solution of TMSOI (1.00 g, 4.55 mmol) in DMSO (10 mL) was added KOtBu (511 mg, 4.55 mmol). The mixture was stirred at 25° C. for 0.5 hour. Then (2R,3R,4S,5S,6R)-3,4,5-tribenzyloxy-2-[(E)-2-diethoxyphosphorylvinyl]-6-phenoxy-tetrahydropyran (1.00 g, 1.52 mmol) in DMSO (10 mL) was added. The mixture was stirred at 25° C. for 12 hours. LC-MS (ESI) found: 673 [M+H]⁺. The mixture was diluted with H₂O₄₀ mL and extracted with EtOAc 150 mL (50 mL* 3). The combined organic layers were washed with brine 50 mL, dried over anhydrous Na₂SO₄, filtered and concentrated under reduced pressure to give a residue. The residue was purified by prep-HPLC (TFA condition) to afford compound (2R,3R,4S,5S,6R)-3,4,5-tribenzyloxy-2-(2-diethoxyphosphorylcyclopropyl)-6-phenoxy-tetrahydropyran (0.12 g, 11.7% yield) as yellow oil. LC-MS (ESI) found: 673 [M+H]⁺.

Step 2: To a solution of (2R,3R,4S,5S,6R)-3,4,5-tribenzyloxy-2-(2-diethoxyphosphorylcyclopropyl)-6-phenoxy-tetrahydropyran (0.12 g, 178 μmol) in DCM (0.5 mL) was added Py (70.5 mg, 891 μmol) and TMSBr (136 mg, 891 μmol). The mixture was stirred at 25° C. for 1 hour. LC-MS (ESI) found: 617 [M+H]⁺. The reaction mixture was diluted with H₂O 10 mL and extracted with DCM 30 mL (10 mL*3), dried over anhydrous Na₂SO₄, filtered and concentrated under reduced pressure to give a residue. The residue was purified by prep-HPLC (Welch Xtimate C18 150*25 mm*5 μm; mobile phase: [water (TFA)−ACN]; B %: 47%-77%, 20 min) to afford compound [2-[(2R,4S,5S,6R)-3,4,5-tribenzyloxy-6-phenoxy-tetrahydropyran-2-yl]cyclopropyl]phosphonic acid (70 mg, 63.6% yield) as yellow oil. ¹H NMR (400 MHz, DMSO-d₆): δ 7.43-7.25 (m, 17H), 7.06-6.98 (m, 3H), 5.65 (d, J=1.6 Hz, 1H), 4.86-4.71 (m, 4H), 4.70-4.59 (m, 2H), 4.08-4.00 (m, 2H), 3.91 (dd, J=3.2, 9.6 Hz, 1H), 3.73 (t, J=9.2 Hz, 2H), 1.54-1.45 (m, 1H), 0.78-0.70 (m, 2H), 0.68-0.59 (m, 1H) and LC-MS (ESI) found: 617 [M+H]⁺.

Step 3: A mixture of [2-[(2R,4S,5S,6R)-3,4,5-tribenzyloxy-6-phenoxy-tetrahydropyran-2-yl]cyclopropyl]phosphonic acid (70 mg, 113 μmol) and Pd(OH)₂/C (79.7 mg, 56.7 μmol, 10% purity) in MeOH (2 mL) was degassed and purged with H₂ (15 Psi) for 3 times, and then the mixture was stirred at 25° C. for 12 hours under H₂ (15 Psi) atmosphere. LC-MS (ESI) found: 347 [M+H]⁺. The mixture was filtered and concentrated under reduced pressure to give a residue. The residue was purified by prep-HPLC (Welch Xtimate C18 150*25 mm*5 μm; mobile phase: [water (TFA)−ACN]; B %: 3%-33%, 10 min) to afford compound [2-[(2R,3S,4S,5S,6R)-3,4,5-trihydroxy-6-phenoxy-tetrahydropyran-2-yl]cyclopropyl]phosphonic acid (6 mg, 15.2% yield) as a white solid. ¹H NMR (400 MHz, MeOD): δ 7.29 (t, J=7.6 Hz, 2H), 7.13-6.96 (m, 3H), 5.43 (s, 1H), 3.99 (s, 1H), 3.87-3.84 (m, 1H), 3.70 (t, J=9.2 Hz, 1H), 3.25-3.21 (m, 1H), 1.68-1.50 (m, 1H), 1.02-0.74 (m, 3H) and LC-MS (ESI) found: 347 [M+H]⁺.

### Preparation of methyl(2-((2R,3S,4S,5S,6R)-3,4,5-trihydroxy-6-phenoxytetrahydro-2H-pyran-2-yl)ethyl)phosphinic acid (A44)

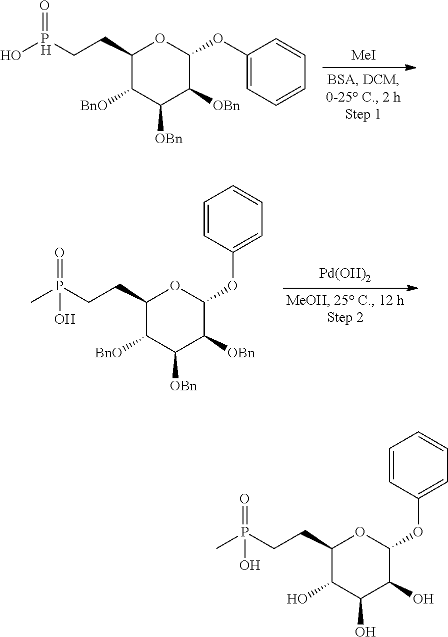

Step 1: A mixture of 2-[(2R,3R,4S,5S,6R)-3,4,5-tribenzyloxy-6-phenoxy-tetrahydropyran-2-yl]ethylphosphinic acid (200 mg, 339 μmol) and Mel (192 mg, 1.36 mmol) in DCM (4 mL) was degassed and purged with N₂ for 3 times, then BSA (483 mg, 2.38 mmol) was added under 0° C. Then the mixture was stirred at 25° C. for 12 hours under N₂ atmosphere. LC-MS (ESI) found: 603 [M+H]⁺. The 1N HCl (10 mL) was added into mixture. The mixture was extracted with EtOAc 60 mL (20 mL*3), and the organic layer was washed with brine (20 mL*3), dried over anhydrous Na₂SO₄, filtered and concentrated under reduced pressure to give a residue. The residue was purified by prep-TLC (SiO₂, DCM: MeOH=20:1) to afford compound methyl-[2-[(2R,3R,4S,5S,6R)-3,4,5-tribenzyloxy-6-phenoxy-tetrahydropyran-2-yl]ethyl]phosphinic acid (100 mg, 48.8% yield) as colorless oil. ¹H NMR (400 MHz, CDCl₃): δ 7.42-7.28 (m, 15H), 7.26-7.23 (m, 2H), 7.04-6.83 (m, 3H), 5.50 (d, J=1.6 Hz, 1H), 5.01-4.58 (m, 6H), 4.09-4.06 (m, 1H), 3.98-3.93 (m, 1H), 3.75 (t, J=9.2 Hz, 1H), 3.62-3.58 (m, 1H), 2.15-2.06 (m, 1H), 1.93-1.67 (m, 2H), 1.56-1.43 (m, 1H), 1.36-1.24 (m, 3H) and LC-MS (ESI) found: 603 [M+H]⁺.

Step 2: A mixture of methyl-[2-[(2R,3R,4S,5S,6R)-3,4,5-tribenzyloxy-6-phenoxy-tetrahydropyran-2-yl]ethyl]phosphinic acid (100 mg, 165 μmol) and Pd(OH)₂/C (116 mg, 20% purity) in MeOH (3 mL) was degassed and purged with H₂ (15 Psi) for 3 times, and then the mixture was stirred at 25° C. for 12 hours under H₂ (15 Psi) atmosphere. LC-MS (ESI) found: 333 [M+H]⁺. The mixture was filtered and concentrated under reduced pressure to afford compound methyl-[2-[(2R,3S,4S,5S,6R)-3,4,5-trihydroxy-6-phenoxy-tetrahydropyran-2-yl]ethyl]phosphinic acid (35 mg, 61.1% yield) as a white solid. ¹H NMR (400 MHz, MeOD): δ 7.27 (t, J=7.6 Hz, 2H), 7.06 (d, J=8.0 Hz, 2H), 6.98 (t, J=7.2 Hz, 1H), 5.46 (d, J=1.2 Hz, 1H), 3.99-3.98 (m, 1H), 3.86 (dd, J=3.6, 9.2 Hz, 1H), 3.57 (t, J=9.6 Hz, 1H), 3.53-3.45 (m, 1H), 2.12-1.99 (m, 1H), 1.78-1.64 (m, 2H), 1.29-1.26 (m, 1H), 1.38 (d, J=13.2 Hz, 3H) and LC-MS (ESI) found: 333 [M+H]⁺.

### Preparation of methyl(2-((2R,3S,4S,5S,6R)-3,4,5-trihydroxy-6-phenoxytetrahydro-2H-pyran-2-yl)ethyl)phosphinic acid (A45)

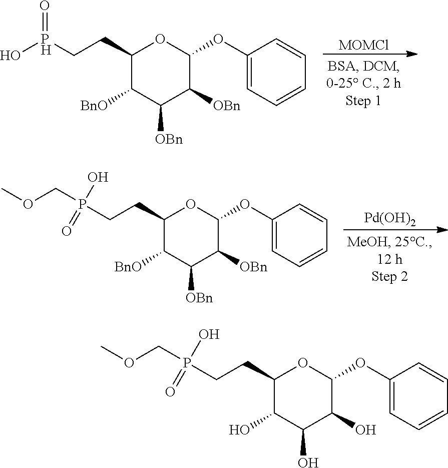

Step 1: A mixture of 2-[(2R,3R,4S,5S,6R)-3,4,5-tribenzyloxy-6-phenoxy-tetrahydropyran-2-yl]ethylphosphinic acid (100 mg, 169 μmol) and MOMCl (190 mg, 2.36 mmol) in DCM (4 mL) was degassed and purged with N₂ for 3 times, then BSA (241 mg, 1.19 mmol) was added under 0° C. Then the mixture was stirred at 25° C. for 2 hours under N₂ atmosphere. LC-MS (ESI) found: 633 [M+H]⁺. The 1 N HCl (10 mL) was added into mixture. The mixture was extracted with EtOAc 60 mL (20 mL*3), and the organic layer was washed with brine (20 mL*3), dried over anhydrous Na₂SO₄, filtered and concentrated under reduced pressure to give a residue. The residue was purified by prep-HPLC (Welch Xtimate C18 150*25 mm*5 μm; mobile phase: [water (TFA)-ACN]; B %: 47%-77%, 10 min) to afford methoxymethyl-[2-[(2R,3R,4S,5S,6R)-3,4,5-tribenzyloxy-6-phenoxy-tetrahydropyran-2-yl]ethyl]phosphinic acid (55 mg, 51.1% yield) as colorless oil. ¹H NMR (400 MHz, CDCl₃): δ 7.42-7.30 (m, 16H), 7.26-7.24 (m, 1H), 7.02-6.92 (m, 3H), 5.50 (d, J=1.2 Hz, 1H), 4.95-4.65 (m, 6H), 4.14-4.06 (m, 1H), 3.96 (s, 1H), 3.80-3.74 (m, 1H), 3.68-3.57 (m, 3H), 3.37 (s, 3H), 2.21-2.09 (m, 1H), 2.00-1.78 (m, 2H), 1.68-1.52 (m, 1H) and LC-MS (ESI) found: 633 [M+H]⁺.

Step 2: A mixture of methoxymethyl-[2-[(2R,3R,4S,5S,6R)-3,4,5-tribenzyloxy-6-phenoxy-tetrahydropyran-2-yl]ethyl]phosphinic acid (55 mg, 86.93 μmol) and Pd(OH)₂/C (30.52 mg, 20% purity) in MeOH (1 mL) was degassed and purged with H₂ (15 Psi) for 3 times, and then the mixture was stirred at 25° C. for 12 hours under H₂ (15 Psi) atmosphere. LC-MS (ESI) found: 363 [M+H]⁺. The mixture was filtered and concentrated under reduced pressure to afford compound methoxymethyl-[2-[(2R,3S,4S,5S,6R)-3,4,5-trihydroxy-6-phenoxy-tetrahydropyran-2-yl]ethyl]phosphinic acid (18 mg, 51.4% yield) as a yellow solid. ¹H NMR (400 MHz, MeOD): δ 7.34-7.24 (m, 2H), 7.07 (d, J=7.6 Hz, 2H), 6.99 (t, J=7.2 Hz, 1H), 5.48 (d, J=1.2 Hz, 1H), 4.00-3.99 (m, 1H), 3.86 (dd, J=3.6, 9.2 Hz, 1H), 3.74-3.60 (m, 2H), 3.62-3.54 (m, 2H), 3.49-3.43 (m, 1H), 3.38 (s, 3H), 2.14-2.04 (m, 1H), 1.90-1.74 (m, 2H), 1.49-1.40 (m, 1H) and LC-MS (ESI) found: 363 [M+H]⁺.

### Preparation of (2-hydroxybenzyl) (2-((2R,3S,4S,5S,6R)-3,4,5-trihydroxy-6-phenoxytetrahydro-2H-pyran-2-yl)ethyl)phosphinic acid (A46)

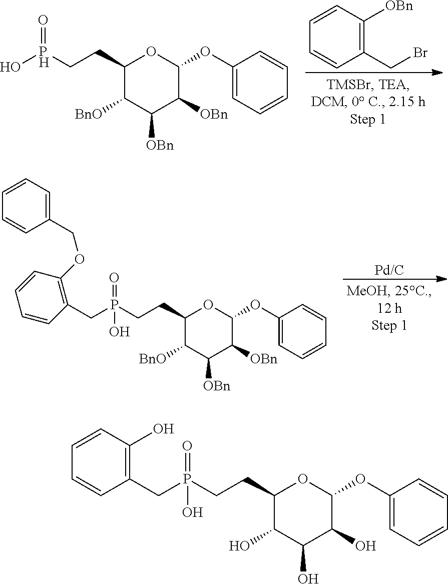

Step 1: A mixture of 2-[(2R,3R,4S,5S,6R)-3,4,5-tribenzyloxy-6-phenoxy-tetrahydropyran-2-yl]ethylphosphinic acid (100 mg, 169 μmol), TMSBr (78.0 mg, 509 μmol) and TEA (51.5 mg, 509 μmol) in DCM (4 mL) was degassed and purged with N₂ for 3 times. Then the mixture was stirred at 0° C. for 15 min under N₂ atmosphere. Then 1-(benzyloxy)-2-(bromomethyl)benzene (47.0 mg, 169 μmol) was added under 0° C. Then the mixture was stirred at 0° C. for 2 hours under N₂ atmosphere. LC-MS (ESI) found: 785 [M+H]⁺. The mixture was concentrated under reduced pressure to give a residue. The residue was purified by prep-HPLC (Welch Xtimate C18 150*25 mm*5 μm; mobile phase: [water (TFA)−ACN]; B %: 62%-92%, 10 min) to afford compound (2-benzyloxyphenyl)methyl-[2-[(2R,3R,4S,5S,6R)-3,4,5-tribenzyloxy-6-phenoxy-tetrahydropyran-2-yl]ethyl]phosphinic acid (50 mg, 37.5% yield) as yellow oil. ¹H NMR (400 MHz, CDCl₃): δ 7.40 -7.28 (m, 14H), 7.26-7.14 (m, 9H), 7.08 (t, J=7.6 Hz, 1H), 6.96 (t, J=7.6 Hz, 1H), 6.88-6.79 (m, 3H), 6.73 (d, J=8.4 Hz, 1H), 5.42 (d, J=1.6 Hz, 1H), 4.90-4.64 (m, 7H), 4.44 (d, J=10.8 Hz, 1H), 4.03-4.00 (m, 1H), 3.94-3.89 (m, 1H), 3.62 (t, J=9.2 Hz, 1H), 3.57-3.51 (m, 1H), 3.17-3.02 (m, 2H), 2.06-1.92 (m, 1H), 1.83-1.61 (m, 2H), 1.44-1.30 (m, 1H) and LC-MS (ESI) found: 785 [M+H]⁺.

Step 2: A mixture of (2-benzyloxyphenyl)methyl-[2-[(2R,3R,4S,5S,6R)-3,4,5-tribenzyloxy-6-phenoxy-tetrahydropyran-2-yl]ethyl]phosphinic acid (30 mg, 38.22 μmol), Pd/C (10 mg, 38.22 μmol, 10% purity) in MeOH (2 mL) was degassed and purged with H₂ (15 Psi) for 3 times, and then the mixture was stirred at 25° C. for 12 hours under H₂ (15 Psi) atmosphere. LC-MS (ESI) found: 425 [M+H]⁺. The mixture was filtered and concentrated under reduced pressure to give a residue. The residue was purified by prep-HPLC (Welch Xtimate C18 150*25 mm*5 μm; mobile phase: [water (TFA)−ACN]; B %: 15%-45%, 10 min) to afford (2-hydroxyphenyl)methyl-[2-[(2R,3S,4S,5S,6R)-3,4,5-trihydroxy-6-phenoxy-tetrahydropyran-2-yl]ethyl]phosphinic acid (6 mg, 36.5% yield) as a white solid. ¹H NMR (400 MHz, MeOD): δ 7.30-7.20 (m, 2H), 7.15 (d, J=7.2 Hz, 1H), 7.08 (t, J=7.6 Hz, 1H), 7.03-6.94 (m, 3H), 6.84-6.75 (m, 2H), 5.43 (d, J=1.6 Hz, 1H), 3.99-3.98 (m, 1H), 3.83 (dd, J=3.2, 9.2 Hz, 1H), 3.50 (t, J=9.6 Hz, 1H), 3.44-3.39 (m, 1H), 3.12 (d, J=18 Hz, 2H), 2.16-2.04 (m, 1H), 1.90-1.65 (m, 2H), 1.45-1.28 (m, 1H) and LC-MS (ESI) found: 425 [M+H]⁺.

### Preparation of (2-hydroxybenzyl) (2-((2R,3S,4S,5S,6R)-3,4,5-trihydroxy-6-phenoxytetrahydro-2H-pyran-2-yl)ethyl)phosphinic acid (A47)

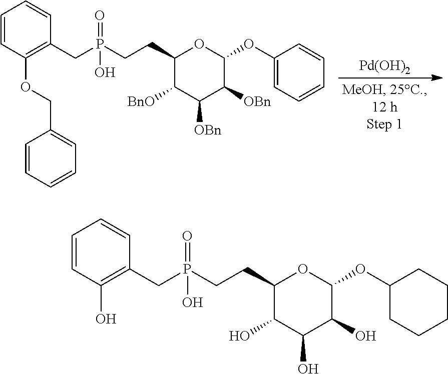

Step 1: A mixture of (2-benzyloxyphenyl)methyl-[2-[(2R,3R,4S,5S,6R)-3,4,5-tribenzyloxy-6-phenoxy-tetrahydropyran-2-yl]ethyl]phosphinic acid (20 mg, 25.4 μmol) and Pd(OH)₂/C (8.95 mg, 20% purity) in MeOH (2 mL) was degassed and purged with H₂ (15 Psi) for 3 times, and then the mixture was stirred at 25° C. for 12 hours under H₂ (15 Psi) atmosphere. LC-MS (ESI) found: 431 [M+H]⁺. The mixture was filtered and concentrated under reduced pressure to give a residue. The residue was purified by prep-HPLC (Welch Xtimate C18 150*25 mm*5 μm; mobile phase: [water (TFA)−ACN]; B %: 17%-47%, 10 min) to afford 2-[(2R,3S,4S,5S,6S)-6-(cyclohexoxy)-3,4,5-trihydroxy-tetrahydropyran-2-yl]ethyl-[(2-hydroxyphenyl)methyl]phosphinic acid (2 mg, 18.2% yield) as a white solid. ¹H NMR (400 MHz, MeOD): δ 7.20 (d, J=7.6 Hz, 1H), 7.07 (t, J=7.6 Hz, 1H), 6.87-6.73 (m, 2H), 4.82 (d, J=1.2 Hz, 1H), 3.74-3.71 (m, 1H), 3.63 (dd, J=3.2, 8.8 Hz, 1H), 3.56-3.39 (m, 3H), 3.19 (d, J=17.6 Hz, 2H), 2.21-2.09 (m, 1H), 2.03-1.91 (m, 1H), 1.84-1.67 (m, 5H), 1.62-1.48 (m, 2H), 1.35-1.23 (m, 5H) and LC-MS (ESI) found: 431 [M+H]⁺.

### Preparation of (cyanomethyl) (2-((2R,3S,4S,5S)-3,4,5-trihydroxy-6-(4-methoxyphenoxy)tetrahydro-2H-pyran-2-yl)ethyl)phosphinic acid (A48)

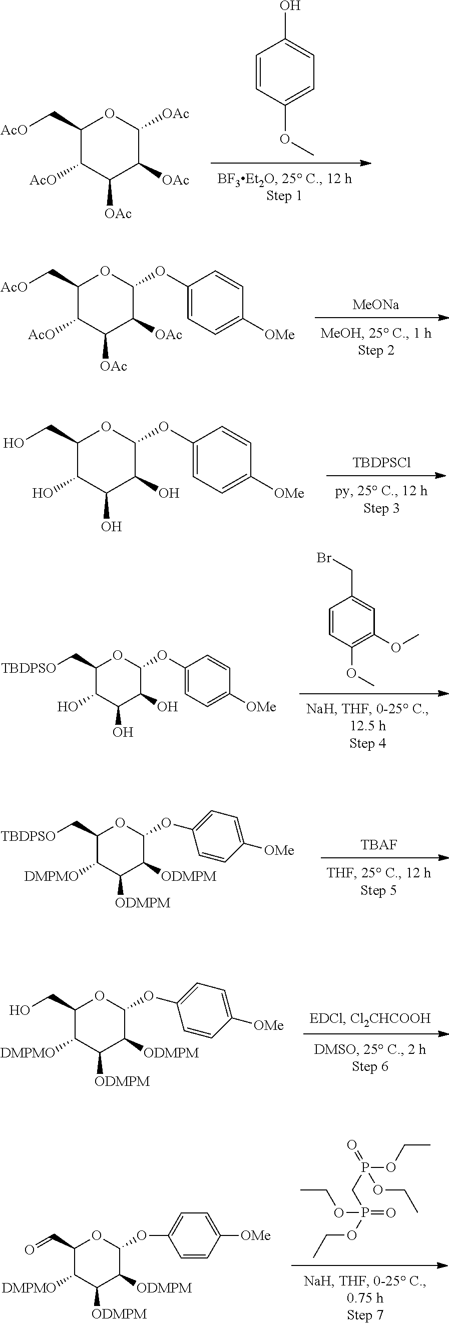

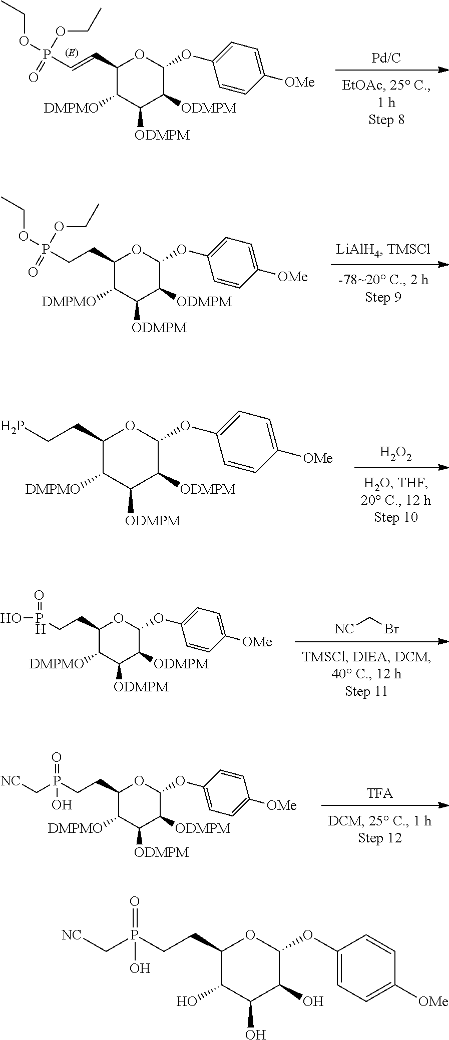

Step 1: To a solution of [(2R,3R,4S,5S,6R)-3,4,5,6-tetraacetoxytetrahydropyran-2-yl]methyl acetate (10.0 g, 25.6 mmol) in DCM (100 mL) was added 4-methoxyphenol (4.77 g, 38.4 mmol) and BF₃·Et₂O(9.09 g, 64.0 mmol). The mixture was stirred at 25° C. for 12 hours. LC-MS (ESI) found: 477 [M+Na]⁺. TLC (petroleum ether:ethyl acetate=2:1) showed [(2R,3R,4S,5S,6R)-3,4,5,6-tetraacetoxytetrahydropyran-2-yl]methyl acetate was consumed completely. Saturated aqueous sodium bicarbonate (100 mL) was added to the reaction mixture, and the reaction mixture was extracted with ethyl acetate (100 mL*3) and washed with brine 50 mL. The organic layer was dried over anhydrous Na₂SO₄ and the filtrate was concentrated in a vacuum evaporator. The residue was purified by column chromatography (SiO₂, petroleum ether:ethyl acetate=2:1) to afford (2R,3R,4S,5S,6R)-2-(acetoxymethyl)-6-(4-methoxyphenoxy)tetrahydro-2H-pyran-3,4,5-triyl triacetate (11.0 g, 94.5% yield) as yellow oil. ¹H NMR (400 MHz, CDCl₃): δ 7.08-6.98 (m, 2H), 6.88-6.77 (m, 2H), 5.56 (dd, J=3.6, 10.0 Hz, 1H), 5.46-5.32 (m, 3H), 4.29 (dd, J=5.2, 12.0 Hz, 1H), 4.19-4.09 (m, 2H), 3.78 (s, 3H), 2.20 (s, 3H), 2.09-2.02 (m, 9H) and LC-MS (ESI) found: 477 [M+Na]⁺.

Step 2: To a solution of (2R,3R,4S,5S,6R)-2-(acetoxymethyl)-6-(4-methoxyphenoxy)tetrahydro-2H-pyran-3,4,5-triyl triacetate (11.0 g, 24.2 mmol) in MeOH (100 mL) was added NaOMe (653 mg, 12.1 mmol). The mixture was stirred at 25° C. for 1 h. LC-MS (ESI) found: 309 [M+Na]⁺. The mixture was adjusted by 1 N HCl to Ph=7, and concentrated under reduced pressure to afford (2R,3S,4S,5S,6R)-2-(hydroxymethyl)-6-(4-methoxyphenoxy)tetrahydropyran-3,4,5-triol (6.30 g, 90.9% yield) as a white solid. LC-MS (ESI) found: 309 [M+H]⁺.

Step 3: To a solution of (2R,3S,4S,5S,6R)-2-(hydroxymethyl)-6-(4-methoxyphenoxy)tetrahydropyran-3,4,5-triol (10.0 g, 34.5 mmol) in Py (100 mL) was added TBDPSCl (14.2 g, 51.8 mmol). The mixture was stirred at 25° C. for 12 hours. LC-MS (ESI) found: 547 [M+Na]⁺. TLC (dichloromethane:methanol=10:1) showed the (2R,3S,4S,5S,6R)-2-(hydroxymethyl)-6-(4-methoxyphenoxy)tetrahydropyran-3,4,5-triol was consumed completely. The mixture was concentrated under reduced pressure to give a residue and the residue was purified by column chromatography (SiO₂, dichloromethane:methanol=10:1) to afford (2R,3S,4S,5S,6R)-2-[[tert-butyl(diphenyl) silyl]oxymethyl]-6-(4-methoxyphenoxy)tetrahydropyran-3,4,5-triol (16.1 g, 86.9% yield) as yellow oil. LC-MS (ESI) found: 547 [M+Na]⁺.

Step 4: To a mixture of (2R,3S,4S,5S,6R)-2-[[tert-butyl(diphenyl) silyl]oxymethyl]-6-(4-methoxyphenoxy)tetrahydropyran-3,4,5-triol (12.0 g, 22.4 mmol) in THF (100 mL) was added NaH (3.59 g, 89.6 mmol, 60% purity) at 0° C. The reaction mixture stirred at 0° C. for 15 min. Then 4-(bromomethyl)-1,2-dimethoxy-benzene (20.7 g, 89.6 mmol) in THF (100 ml) was added, the reaction mixture and stirred at 25° C. for 12 hours. LC-MS (ESI) found: 997 [M+Na]⁺. TLC (petroleum ether:ethyl acetate=1:1) showed (2R,3S,4S,5S,6R)-2-(hydroxymethyl)-6-(4-methoxyphenoxy)tetrahydropyran-3,4,5-triol was consumed completely. The mixture was quenched by addition NH₄Cl aqueous solution (100 mL), extracted with EtOAc (200 mL*3), the combined organic layers were washed with brine (100 mL), dried over anhydrous Na₂SO₄, filtered and concentrated under reduced pressure. The residue was purified by column chromatography (SiO₂, petroleum ether:ethyl acetate=1:1) to afford tert-butyl-diphenyl-[[(2R,3R,4S,5S,6R)-3,4,5-tris[(3,4-dimethoxyphenyl)methoxy]-6-(4-methoxyphenoxy)tetrahydropyran-2-yl]methoxy]silane (8.00 g, 36.6% yield) as yellow oil. ¹H NMR (400 MHz, CDCl₃): δ 7.73-7.64 (m, 4H), 7.43-7.29 (m, 6H), 6.97-6.90 (m, 6H), 6.84-6.70 (m, 7H), 5.39 (d, J=1.6 Hz, 1H), 4.89 (d, J=10.4 Hz, 1H), 4.79-4.72 (m, 1H), 4.71-4.63 (m, 3H), 4.58 (d, J=10.4 Hz, 1H), 4.11 (s, 1H), 3.98-3.91 (m, 3H), 3.89 (s, 4H), 3.86 (d, J=4.0 Hz, 6H), 3.83-3.77 (m, 7H), 3.74 (s, 3H), 3.70 (s, 3H), 1.03 (s, 9H)) and LC-MS (ESI) found: 997 [M+Na]⁺.

Step 5: To a solution of tert-butyl-diphenyl-[[(2R,3R,4S,5S,6R)-3,4,5-tris[(3,4-dimethoxyphenyl)methoxy]-6-(4-methoxyphenoxy)tetrahydropyran-2-yl]methoxy]silane (8.00 g, 8.20 mmol) in THF (50 mL) was added TBAF (1 M, 16.4 mL). The mixture was stirred at 25° C. for 12 hours. LC-MS (ESI) found: 759 [M+Na]⁺. TLC (petroleum ether:ethyl acetate=1:1) showed tert-butyl-diphenyl-[[(2R,3R,4S,5S,6R)-3,4,5-tris[(3,4-dimethoxyphenyl)methoxy]-6-(4-methoxyphenoxy)tetrahydropyran-2-yl]methoxy]silane was remained. The mixture was diluted with H₂O₂₀ mL and extracted with EtOAc 90 mL (30 mL*3). The combined organic layers were washed with brine 30 mL, dried over anhydrous Na₂SO₄, filtered and concentrated under reduced pressure to give a residue. The residue was purified by column chromatography (SiO₂, petroleum ether:ethyl acetate=1:1) to afford [(2R,3R,4S,5S,6R)-3,4,5-tris[(3,4-dimethoxyphenyl)methoxy]-6-(4-methoxyphenoxy)tetrahydropyran-2-yl]methanol (5.40 g, 89.3% yield) as yellow oil. LC-MS (ESI) found: 759 [M+Na]⁺.

Step 6: To a solution of [(2R,3R,4S,5S,6R)-3,4,5-tris[(3,4-dimethoxyphenyl)methoxy]-6-(4-methoxyphenoxy)tetrahydropyran-2-yl]methanol (9.60 g, 13.0 mmol) in DMSO (100 mL) was added EDCI (8.49 g, 44.3 mmol) and 2,2-dichloroacetic acid (840 mg, 6.51 mmol). The mixture was stirred at 25° C. for 2 hours. TLC (petroleum ether:ethyl acetate=1:2) showed [(2R,3R,4S,5S,6R)-3,4,5-tris[(3,4-dimethoxyphenyl)methoxy]-6-(4-methoxyphenoxy)tetrahydropyran-2-yl]methanol was consumed completely. The reaction mixture was quenched by addition H₂O₁₀₀ mL at 0° C., then extracted with EtOAc 600 mL (200 mL*3). The combined organic layers were washed with brine 200 mL, dried over anhydrous Na₂SO₄, filtered and concentrated under reduced pressure afford (2S,3S,4S,5S,6R)-3,4,5-tris[(3,4-dimethoxyphenyl)methoxy]-6-(4-methoxyphenoxy)tetrahydropyran-2-carbaldehyde (11.0 g, crude) as yellow oil.

Step 7: To a solution of 1-[diethoxyphosphorylmethyl(ethoxy)phosphoryl]oxyethane (6.47 g, 22.4 mmol) in THF (60 mL) was added NaH (898 mg, 22.4 mmol, 60% purity) under 0° C. The mixture was stirred at 0° C. for 15 min. Then (2S,3S,4S,5S,6R)-3,4,5-tris[(3,4-dimethoxyphenyl)methoxy]-6-(4-methoxyphenoxy)tetrahydropyran-2-carbaldehyde (11.0 g, 14.9 mmol) in THF (60 mL) was added. The mixture was stirred at 25° C. for 0.5 hour. LC-MS (ESI) found: 869 [M+H]⁺. TLC (petroleum ether:ethyl acetate=0:1) showed the desired spot was detected. The mixture was quenched by saturated NH₄Cl aqueous solution (100 mL) at 0° C., extracted with EtOAc (200 mL* 3), the combined organic phase washed with brine (200 mL), dried over anhydrous Na₂SO₄, filtered and concentrated under reduced pressure. The residue was purified by column chromatography (SiO₂, petroleum ether:ethyl acetate=0:1) to afford (2R,3R,4S,5S,6R)-2-[(E)-2-diethoxyphosphorylvinyl]-3,4,5-tris[(3,4-dimethoxyphenyl)methoxy]-6-(4-methoxyphenoxy)tetrahydropyran (6.10 g, 46.9% yield) as yellow oil. LC-MS (ESI) found: 869 [M+H]⁺.

Step 8: A mixture of (2R,3R,4S,5S,6R)-2-[(E)-2-diethoxyphosphorylvinyl]-3,4,5-tris[(3,4-dimethoxyphenyl)methoxy]-6-(4-methoxyphenoxy)tetrahydropyran (4.10 g, 4.72 mmol) and Pd/C (400 mg, 10% purity) in EtOAc (50 mL) was degassed and purged with H₂ (15 Psi) for 3 times, and then the mixture was stirred at 25° C. for 1 hour under H₂ (15 Psi) atmosphere. LC-MS (ESI) found: 871 [M+H]⁺. The mixture was filtered and concentrated under reduced pressure to afford (2R,3R,4S,5S,6R)-2-(2-diethoxyphosphorylethyl)-3,4,5-tris[(3,4-dimethoxyphenyl)methoxy]-6-(4-methoxyphenoxy)tetrahydropyran (4.00 g, 97.3% yield) as colorless oil.

Step 9: To a solution of LAH (2.61 g, 68.8 mmol) in THF (40 mL) was degassed and purged with N₂ for 3 times, then TMSCl (5.99 g, 55.1 mmol) was added under−78° C. and the mixture was stirred at 25° C. for 1 h under N₂ atmosphere. Then (2R,3R,4S,5S,6R)-2-(2-diethoxyphosphorylethyl)-3,4,5-tris[(3,4-dimethoxyphenyl)methoxy]-6-(4-methoxyphenoxy)tetrahydropyran (4.00 g, 4.59 mmol) in THF (40 mL) was added under 0° C. The mixture was stirred at 25° C. for 1 h under N₂ atmosphere. LC-MS (ESI) found: 767 [M+H] The reaction was quenched with H₂O (100 mL) at 0° C. and then diluted with EtOAc 100 mL and filtered. The mixture was extracted with EtOAc 600 mL (200 mL*3). The combined organic layers were washed with brine 200 mL, dried over anhydrous Na₂SO₄, filtered and concentrated under reduced pressure to afford 2-[(2R,3R,4S,5S,6R)-3,4,5-tris[(3,4-dimethoxyphenyl)methoxy]-6-(4-methoxyphenoxy)tetrahydropyran-2-yl]ethylphosphane (3.00 g, 85.18% yield) as colorless oil.

Step 10: To a solution of 2-[(2R,3R,4S,5S,6R)-3,4,5-tris[(3,4-dimethoxyphenyl)methoxy]-6-(4-methoxyphenoxy)tetrahydropyran-2-yl]ethylphosphane (3.00 g, 3.91 mmol) in THF (15 mL) and H₂O (15 mL) was added H₂O₂ (6.02 g, 53.09 mmol). The mixture was stirred at 25° C. for 12 hours. LC-MS (ESI) found: 799 [M+H]⁺. The reaction mixture was quenched by addition saturated NaHSO₃ aqueous solution 100 mL at 0° C. and extracted with EtOAc 300 mL (100 mL*3). The combined organic layers were washed with addition saturated NaHSO₃ aqueous solution (100 mL* 3), dried over anhydrous Na₂SO₄, filtered and concentrated under reduced pressure to afford 2-[(2R,3R,4S,5S,6R)-3,4,5-tris[(3,4-dimethoxyphenyl)methoxy]-6-(4-methoxyphenoxy)tetrahydropyran-2-yl]ethylphosphinic acid (2.50 g, 80.0% yield) as colorless oil.

Step 11: A mixture of 2-[(2R,3R,4S,5S,6R)-3,4,5-tris[(3,4-dimethoxyphenyl)methoxy]-6-(4-methoxyphenoxy)tetrahydropyran-2-yl]ethylphosphinic acid (50 mg, 62.5 μmol), TMSCl (13.6 mg, 125.1 μmol) and DIEA (16.1 mg, 125 μmol) in DCM (2 mL) was degassed and purged with N₂ for 3 times, then 2-bromoacetonitrile (0.11 g, 917 μmol) and DIEA (8.09 mg, 62.5 μmol) were added. Then the mixture was stirred at 40° C. for 12 hours under N₂ atmosphere. LC-MS (ESI) found: 838 [M+H]⁺. The reaction mixture was diluted with EtOAc 20 mL. The combined organic layers were washed with NH₄Cl aqueous solution 10 mL, H₂O₁₀ mL and brine 10 mL, dried over anhydrous Na₂SO₄, filtered and concentrated under reduced pressure to give a residue. The residue was purified by prep-HPLC (Welch Xtimate C18 150*25 mm*5 μm; mobile phase: [water (TFA)−ACN];gradient: 40%-70% B over 10 min) to afford cyanomethyl-[2-[(2R,3R,4S,5S)-3,4,5-tris[(3,4-dimethoxyphenyl)methoxy]-6-(4-methoxyphenoxy)tetrahydropyran-2-yl]ethyl]phosphinic acid (15 mg, 28.6% yield) as a yellow solid. ¹H NMR (400 MHz, CDCl₃): δ 6.95-6.79 (m, 13H), 5.35 (s, 1H), 4.93-4.62 (m, 6H), 4.06 (dd, J=2.4, 9.2 Hz, 1H), 3.92 (d, J=2.4 Hz, 1H), 3.90 (s, 3H), 3.87 (s, 3H), 3.85 (s, 3H), 3.82-3.78 (m, 9H), 3.75 (s, 4H), 3.66-3.60 (m, 1H), 2.72 (d, J=15.6 Hz, 2H), 2.13-1.99 (m, 1H), 1.94-1.81 (m, 1H), 1.79-1.59 (m, 2H) and LC-MS (ESI) found: 838 [M+H]⁺.

Step 12: To a solution of cyanomethyl-[2-[(2R,3R,4S,5S)-3,4,5-tris[(3,4-dimethoxyphenyl)methoxy]-6-(4-methoxyphenoxy)tetrahydropyran-2-yl]ethyl]phosphinic acid (15 mg, 17.9 μmol) in TFA (1 mL) and DCM (1 mL). The mixture was stirred at 25° C. for 1 hour. LC-MS (ESI) found: 388 [M+H]⁺. The mixture was concentrated under reduced pressure to give a residue. The residue was purified by prep-HPLC (Welch Xtimate C18 150*25 mm*5 μm; mobile phase: [water (TFA)−ACN]; gradient: 1%-31% B over 10 min) to afford cyanomethyl-[2-[(2R,3S,4S,5S)-3,4,5-trihydroxy-6-(4-methoxyphenoxy)tetrahydropyran-2-yl]ethyl]phosphinic acid (1.9 mg, 26.44% yield) was obtained as a white solid. ¹H NMR (400 MHz, MeOD): δ 7.06-6.95 (m, 2H), 6.90-6.82 (m, 2H), 5.38 (d, J=1.6 Hz, 1H), 4.00 (dd, J=1.6, 3.2 Hz, 1H), 3.89-3.81 (m, 1H), 3.75 (s, 3H), 3.61-3.52 (m, 2H), 2.99 (d, J=15.6 Hz, 2H), 2.23-2.10 (m, 1H), 2.08-1.95 (m, 1H), 1.83-1.54 (m, 2H) and LC-MS (ESI) found: 388 [M+H]⁺.

### Preparation of methyl(2-cyanobenzyl) (2-((2R,3S,4S,5S)-3,4,5-trihydroxy-6-(4-methoxyphenoxy)tetrahydro-2H-pyran-2-yl)ethyl)phosphinic acid (A49)

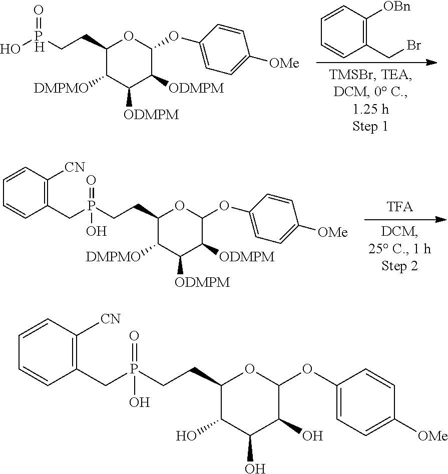

Step 1: A mixture of 2-[(2R,3R,4S,5S,6R)-3,4,5-tris[(3,4-dimethoxyphenyl)methoxy]-6-(4-methoxyphenoxy)tetrahydropyran-2-yl]ethylphosphinic acid (45.0 mg, 56.3 μmol), TMSBr (43.1 mg, 281 μmol) and TEA (28.5 mg, 281 μmol) in DCM (2 mL) was degassed and purged with N₂ for 3 times. Then the mixture was stirred at 0° C. for 15 min under N₂ atmosphere. Then 2-(bromomethyl)benzonitrile (11.0 mg, 56.37 μmol) was added under 0° C. Then the mixture was stirred at 0° C. for 1 hour under N₂ atmosphere. LC-MS (ESI) found: 914 [M+H]⁺. The mixture was diluted with H₂O₁₀ mL, extracted with DCM (20 mL*3), dried over anhydrous Na₂SO₄, filtered and concentrated under reduced pressure. The residue was purified by prep-HPLC (Welch Xtimate C18 150*25 mm*5 μm; mobile phase: [water (TFA)−ACN]; gradient: 40%-70% B over 10 min) to afford (2-cyanophenyl)methyl-[2-[(2R,3R,4S,5S)-3,4,5-tris[(3,4-dimethoxyphenyl)methoxy]-6-(4-methoxyphenoxy)tetrahydropyran-2-yl]ethyl]phosphinic acid (10 mg, 19.41% yield) as a white solid. ¹H NMR (400 MHz, CDCl₃): δ 7.47 (d, J=7.6 Hz, 1H), 7.43-7.30 (m, 2H), 7.16 (t, J=7.2 Hz, 1H), 7.08-6.62 (m, 12H), 5.34 (d, J=1.2 Hz, 1H), 4.89-4.54 (m, 6H), 4.04 (dd, J=2.8, 8.8 Hz, 1H), 3.97-3.64 (m, 23H), 3.59-3.54 (m, 1H), 3.15 (d, J=16.4 Hz, 2H), 2.11-1.94 (m, 1H), 1.84-1.58 (m, 2H), 1.42-1.26 (m, 1H) and LC-MS (ESI) found: 914 [M+H]⁺.

Step 2: A solution of (2-cyanophenyl)methyl-[2-[(2R,3R,4S,5S)-3,4,5-tris[(3,4-dimethoxyphenyl)methoxy]-6-(4-methoxyphenoxy)tetrahydropyran-2-yl]ethyl]phosphinic acid (20 mg, 21.8 μmol) in TFA (1 mL) and DCM (1 mL) was stirred at 25° C. for 1 hour. LC-MS (ESI) found: 493 [M+Na]⁺. The mixture was concentrated under reduced pressure to give a residue. The residue was purified by prep-HPLC (Welch Xtimate C18 150*25 mm*5 μm; mobile phase: [water (TFA)−ACN]; gradient: 15%-45% B over 10 min) to afford (2-cyanophenyl)methyl-[2-[(2R,3S,4S,5S)-3,4,5-trihydroxy-6-(4-methoxyphenoxy)tetrahydropyran-2-yl]ethyl]phosphinic acid (3 mg, 28.7% yield) as a white solid. ¹H NMR (400 MHz, MeOD): δ 7.69 (d, J=7.6 Hz, 1H), 7.61-7.56 (m, 1H), 7.52-7.47 (m, 1H), 7.45-7.37 (m, 1H), 6.97 (d, J=8.8 Hz, 2H), 6.85 (d, J=8.8 Hz, 2H), 5.36 (s, 1H), 3.99 (dd, J=1.6, 3.2 Hz, 1H), 3.83 (dd, J=3.2, 8.8 Hz, 1H), 3.76 (s, 3H), 3.54-3.46 (m, 2H), 3.35 (d, J=16.8 Hz, 2H), 2.20-2.06 (m, 1H), 1.95-1.66 (m, 2H), 1.54-1.38 (m, 1H) and LC-MS (ESI) found: 493 [M+Na]⁺.

### Preparation of (pyridin-2-ylmethyl) (2-((2R,3S,4S,5S)-3,4,5-trihydroxy-6-(4-methoxyphenoxy)tetrahydro-2H-pyran-2-yl)ethyl)phosphinic acid (A50)

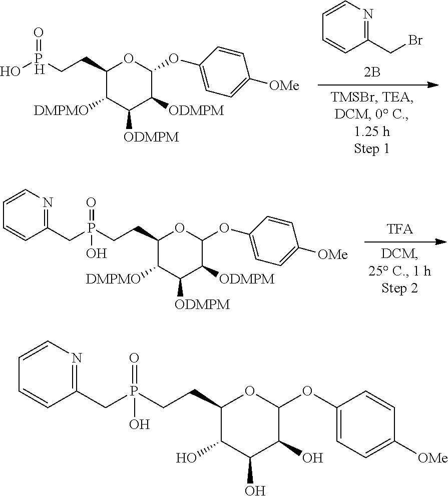

Step 1: A mixture of 2-[(2R,3R,4S,5S,6R)-3,4,5-tris[(3,4-dimethoxyphenyl)methoxy]-6-(4-methoxyphenoxy)tetrahydropyran-2-yl]ethylphosphinic acid (50 mg, 62.5 μmol), TMSBr (47.9 mg, 312 μmol) and TEA (31.6 mg, 312 μmol) in DCM (4 mL) was degassed and purged with N₂ for 3 times. Then the mixture was stirred at 0° C. for 15 min under N₂ atmosphere. Then 2-(bromomethyl)pyridine (21.5 mg, 125 μmol) was added under 0° C. Then the mixture was stirred at 0° C. for 1 hour under N₂ atmosphere. LC-MS (ESI) found: 890 [M+H]⁺. The mixture was diluted with H₂O₁₀ mL, extracted with DCM (20 mL*3), dried over anhydrous Na₂SO₄, filtered and concentrated under reduced pressure. The residue was purified by prep-HPLC (Phenomenex luna C18 150*25 mm*10 μm; mobile phase: [water (TFA)−ACN]; gradient: 30%-60% B over) to afford 2-pyridylmethyl-[2-[(2R,3R,4S,5S)-3,4,5-tris[(3,4-dimethoxyphenyl)methoxy]-6-(4-methoxyphenoxy)tetrahydropyran-2-yl]ethyl]phosphinic acid (33 mg, 59.2% yield) as a white solid. ¹H NMR (400 MHz, CDCl₃): δ 8.53 (d, J=5.2 Hz, 1H), 8.06 (t, J=7.2 Hz, 1H), 7.66-7.49 (m, 2H), 6.97-6.79 (m, 13H), 5.36 (s, 1H), 4.90-4.59 (m, 6H), 4.05 (dd, J=2.4, 8.8 Hz, 1H), 3.94-3.86 (m, 7H), 3.83-3.57 (m, 17H), 3.41 (d, J=14.8 Hz, 2H), 2.17-2.01 (m, 1H), 1.92-1.77 (m, 1H), 1.75-1.60 (m, 1H), 1.57-1.39 (m, 1H) and LC-MS (ESI) found: 890 [M+H]⁺.

Step 2: A solution of 2-pyridylmethyl-[2-[(2R,3R,4S,5S)-3,4,5-tris[(3,4-dimethoxyphenyl) methoxy]-6-(4-methoxyphenoxy)tetrahydropyran-2-yl]ethyl]phosphinic acid (33 mg, 37.0 μmol) in TFA (1 mL) and DCM (1 mL) was stirred at 25° C. for 1 hour. LC-MS (ESI) found: 440 [M+H]⁺. The mixture was concentrated under reduced pressure to give a residue. The residue was purified by prep-HPLC (Phenomenex luna C18 150*25 mm*10 μm; mobile phase: [water (TFA)−ACN]; gradient: 0%-28% B over 15 min) to afford 2-pyridylmethyl-[2-[(2R,3S,4S,5S)-3,4,5-trihydroxy-6-(4-methoxyphenoxy)tetrahydropyran-2-yl]ethyl]phosphinic acid (5 mg, 30.0% yield) as a white solid. ¹H NMR (400 MHz, MeOD): δ 8.63 (d, J=5.2 Hz, 1H), 8.34 (t, J=7.2 Hz, 1H), 7.86-7.72 (m, 2H), 7.05-6.92 (m, 2H), 6.87-6.76 (m, 2H), 5.37 (d, J=1.6 Hz, 1H), 4.00 (dd, J=1.6, 3.2 Hz, 1H), 3.84 (dd, J=3.2, 8.8 Hz, 1H), 3.75 (s, 3H), 3.58-3.51 (m, 2H), 3.43 (d, J=15.2 Hz, 2H), 2.19-2.05 (m, 1H), 1.87-1.65 (m, 2H), 1.56-1.36 (m, 1H) and LC-MS (ESI) found: 440 [M+H]⁺.

### Preparation of (2-chloro-6-fluorobenzyl) (2-((2R,3S,4S,5S,6R)-3,4,5-trihydroxy-6-(4-methoxyphenoxy)tetrahydro-2H-pyran-2-yl)ethyl)phosphinic acid (A51)

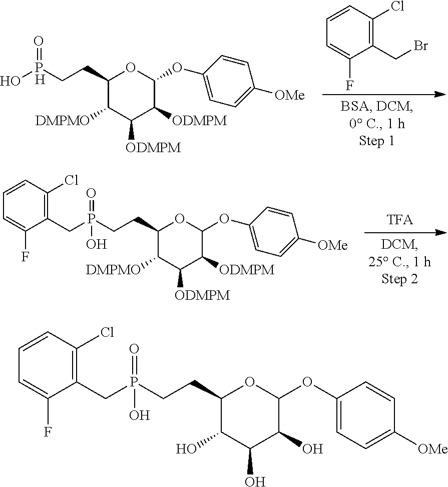

Step 1: A mixture of 2-[(2R,3R,4S,5S,6R)-3,4,5-tris[(3,4-dimethoxyphenyl)methoxy]-6-(4-methoxyphenoxy)tetrahydropyran-2-yl]ethylphosphinic acid (50 mg, 62.5 μmol), 2-(bromomethyl)-1-chloro-3-fluoro-benzene (20.9 mg, 93.8 μmol) and BSA (89.1 mg, 438 μmol) in DCM (2 mL) was degassed and purged with N₂ for 3 times, and then the mixture was stirred at 0° C. for 1 hour under N₂ atmosphere. LC-MS (ESI) found: 941 [M+H]⁺. The mixture was diluted with H₂O₁₀ mL, extracted with DCM (20 mL*3), dried over anhydrous Na₂SO₄, filtered and concentrated under reduced pressure. The residue was purified by prep-HPLC (Waters xbridge 150*25 mm 10 μm; mobile phase: [water (NH₄HCO₃)−ACN]; gradient: 30%-60% B over) to afford (2-chloro-6-fluoro-phenyl)methyl-[2-[(2R,3R,4S,5S)-3,4,5-tris[(3,4-dimethoxyphenyl)methoxy]-6-(4-methoxyphenoxy)tetrahydropyran-2-yl]ethyl]phosphinic acid (20 mg, crude) as a white solid ¹H NMR (400 MHz, CDCl₃): δ 7.12-6.98 (m, 2H), 6.93-6.76 (m, 14H), 5.34 (s, 1H), 4.89-4.57 (m, 6H), 4.04 (dd, J=2.4, 8.8 Hz, 1H), 3.91-3.85 (m, 7H), 3.82 (s, 3H), 3.80-3.67 (m, 13H), 3.63-3.57 (m, 1H), 3.21 (d, J=17.2 Hz, 2H), 2.20-2.09 (m, 1H), 1.92-1.72 (m, 2H), 1.50-1.38 (m, 1H) and LC-MS (ESI) found: 941 [M+H]⁺.

Step 2: A solution of (2-chloro-6-fluoro-phenyl)methyl-[2-[(2R,3R,4S,5S)-3,4,5-tris[(3,4-dimethoxyphenyl)methoxy]-6-(4-methoxyphenoxy)tetrahydropyran-2-yl]ethyl]phosphinic acid (60 mg, 63.7 μmol) in TFA (2 mL) and DCM (2 mL) was stirred at 25° C. for 1 hour. LC-MS (ESI) found: 491 [M+H]⁺. The mixture was concentrated under reduced pressure to give a residue. The residue was purified by prep-HPLC (Welch Xtimate C18 150*25 mm*5 μm; mobile phase: [water (TFA)−ACN]; gradient: 15%-45% B over 10 min) to afford (2-chloro-6-fluoro-phenyl)methyl-[2-[(2R,3S,4S,5S)-3,4,5-trihydroxy-6-(4-methoxyphenoxy)tetrahydropyran-2-yl]ethyl]phosphinic acid (14.2 mg, 45.3% yield) as a white solid. ¹H NMR (400 MHz, MeOD): δ 7.31-7.22 (m, 2H), 7.14-7.04 (m, 1H), 7.01-6.94 (m, 2H), 6.89-6.78 (m, 2H), 5.36 (d, J=1.6 Hz, 1H), 3.99 (dd, J=1.6, 3.2 Hz, 1H), 3.83 (dd, J=3.2, 8.8 Hz, 1H), 3.76 (s, 3H), 3.53-3.47 (m, 2H), 3.39-3.32 (m, 2H), 2.22-2.08 (m, 1H), 1.98-1.83 (m, 1H), 1.78-1.65 (m, 1H), 1.57-1.40 (m, 1H) and LC-MS (ESI) found: 491 [M+H]⁺.

### Preparation of allyl(2-((2R,3S,4S,5S)-3,4,5-trihydroxy-6-(4-methoxyphenoxy)tetrahydro-2H-pyran-2-yl)ethyl)phosphinic acid (A52)

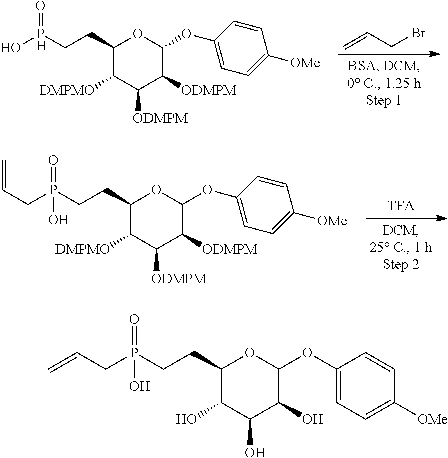

Step 1: A mixture of 2-[(2R,3R,4S,5S,6R)-3,4,5-tris[(3,4-dimethoxyphenyl)methoxy]-6-(4-methoxyphenoxy)tetrahydropyran-2-yl]ethylphosphinic acid (50 mg, 62.5 μmol) and 3-bromoprop-1-ene (22.7 mg, 187 μmol) in DCM (2 mL) was degassed and purged with N₂ for 3 times, then BSA (89.1 mg, 438 μmol) was added under 0° C. Then the mixture was stirred at 0° C. for 1 hour under N₂ atmosphere. LC-MS (ESI) found: 839 [M+H]⁺. The mixture was diluted with H₂O₁₀ mL, extracted with DCM (20 mL*3), dried over anhydrous Na₂SO₄, filtered and concentrated under reduced pressure. The residue was purified by prep-HPLC (Welch Xtimate C18 150*25 mm*5 μm; mobile phase: [water (TFA)−ACN]; gradient: 37%-67% B over 10 min) to afford allyl-[2-[(2R,3R,4S,5S)-3,4,5-tris[(3,4-dimethoxyphenyl)methoxy]-6-(4-methoxyphenoxy)tetrahydropyran-2-yl]ethyl]phosphinic acid (25 mg, 47.6% yield) as a white solid. ¹H NMR (400 MHz, CDCl₃): δ 6.96-6.91 (m, 3H), 6.88-6.79 (m, 10H), 5.80-5.57 (m, 1H), 5.35 (s, 1H), 5.22-5.12 (m, 2H), 4.89-4.58 (m, 6H), 4.08-4.02 (m, 1H), 3.92 (s, 1H), 3.90-3.86 (m, 10H), 3.80 (d, J=5.2 Hz, 9H), 3.76 (s, 4H), 3.65-3.61 (m, 1H), 2.55-2.48 (m, 2H), 2.15-2.07 (m, 1H), 1.86-1.73 (m, 2H), 1.53-1.46 (m, 1H) and LC-MS (ESI) found: 839 [M+H]⁺.

Step 2: A solution of allyl-[2-[(2R,3R,4S,5S)-3,4,5-tris[(3,4-dimethoxyphenyl)methoxy]-6-(4-methoxyphenoxy)tetrahydropyran-2-yl]ethyl]phosphinic acid (15 mg, 17.8 μmol) in TFA (1 mL) and DCM (1 mL) was stirred at 25° C. for 1 hour. LC-MS (ESI) found: 389 [M+H]⁺. The mixture was concentrated under reduced pressure to give a residue. The residue was purified by prep-HPLC (Welch Ultimate C18 150*25 mm*5 μm; mobile phase: [water (TFA)−ACN]; gradient: 8%-38% B over 10 min) to afford allyl-[2-[(2R,3S,4S,5S)-3,4,5-trihydroxy-6-(4-methoxyphenoxy)tetrahydropyran-2-yl]ethyl]phosphinic acid (6 mg, 79.4% yield) as a white solid.

¹H NMR (400 MHz, MeOD): δ 7.04-6.95 (m, 2H), 6.91-6.81 (m, 2H), 5.84-5.70 (m, 1H), 5.38 (d, J=1.6 Hz, 1H), 5.24-5.13 (m, 2H), 3.99 (dd, J=1.6, 3.2 Hz, 1H), 3.83 (dd, J=3.2, 8.8 Hz, 1H), 3.75 (s, 3H), 3.55-3.47 (m, 2H), 2.54 (dd, J=7.6, 17.6 Hz, 2H), 2.18-2.03 (m, 1H), 1.90-1.75 (m, 1H), 1.75-1.63 (m, 1H), 1.51-1.37 (m, 1H) and LC-MS (ESI) found: 389 [M+H]⁺.

### Preparation of but-2-yn-1-yl(2-((2R,3S,4S,5S)-3,4,5-trihydroxy-6-(4-methoxyphenoxy)tetrahydro-2H-pyran-2-yl)ethyl)phosphinic acid (A53)

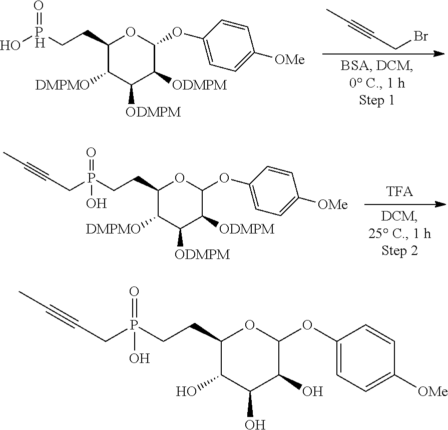

Step 1: A mixture of 2-[(2R,3R,4S,5S,6R)-3,4,5-tris[(3,4-dimethoxyphenyl)methoxy]-6-(4-methoxyphenoxy)tetrahydropyran-2-yl]ethylphosphinic acid (100 mg, 125 μmol) and 1-bromobut-2-yne (33.3 mg, 250 μmol) in DCM (4 mL) was degassed and purged with N₂ for 3 times, then BSA (178 mg, 876 μmol) was added under 0° C. Then the mixture was stirred at 25° C. for 1 hour under N₂ atmosphere. LC-MS (ESI) found: 851 [M+H]⁺. The mixture was diluted with H₂O₁₀ mL, extracted with DCM (20 mL*3), dried over anhydrous Na₂SO₄, filtered and concentrated under reduced pressure. The residue was purified by prep-HPLC (Welch Xtimate C18 150*25 mm*5 μm; mobile phase: [water (TFA)−ACN]; gradient: 38%-68% B over 10 min) to afford but-2-ynyl-[2-[(2R,3R,4S,5S)-3,4,5-tris[(3,4-dimethoxyphenyl)methoxy]-6-(4-methoxyphenoxy)tetrahydropyran-2-yl]ethyl]phosphinic acid (54 mg, 50.7% yield) as a white solid. ¹H NMR (400 MHz, CDCl₃): δ 6.95-6.78 (m, 13H), 5.35 (s, 1H), 4.92-4.59 (m, 6H), 4.09-4.03 (m, 1H), 3.91 (d, J=2.8 Hz, 1H), 3.90-3.84 (m, 9H), 3.80 (d, J=7.2 Hz, 9H), 3.76 (s, 4H), 3.67-3.62 (m, 1H), 2.64-2.53 (m, 2H), 2.28-1.76 (m, 4H), 1.70 (s, 3H) and LC-MS (ESI) found: 851 [M+H]⁺.

Step 2: To a solution of but-2-ynyl-[2-[(2R,3R,4S,5S)-3,4,5-tris[(3,4-dimethoxyphenyl)methoxy]-6-(4-methoxyphenoxy)tetrahydropyran-2-yl]ethyl]phosphinic acid (44 mg, 51.7 μmol) in TFA (2 mL) and DCM (2 mL). The mixture was stirred at 25° C. for 1 hour. LC-MS (ESI) found: 401 [M+H]⁺. The mixture was concentrated under reduced pressure to give a residue. The residue was purified by prep-HPLC (Phenomenex luna C18 150*25 mm*10 μm; mobile phase: [water (TFA)−ACN]; gradient: 8%-38% B over 15 min) to afford but-2-ynyl-[2-[(2R,3S,4S,5S)-3,4,5-trihydroxy-6-(4-methoxyphenoxy)tetrahydropyran-2-yl]ethyl]phosphinic acid (9.5 mg, 43.7% yield) as a white solid. ¹H NMR (400 MHz, MeOD): δ 7.05-6.96 (m, 2H), 6.89-6.81 (m, 2H), 5.38 (d, J=1.6 Hz, 1H), 4.00 (dd, J=1.6, 3.2 Hz, 1H), 3.84 (dd, J=3.6, 8.8 Hz, 1H), 3.75 (s, 3H), 3.57-3.51 (m, 2H), 2.60 (dd, J=2.4, 17.6 Hz, 2H), 2.22-2.08 (m, 1H), 2.06-1.95 (m, 1H), 1.77-1.52 (m, 5H) and LC-MS (ESI) found: 401 [M+H]⁺.

### Preparation of (2,3,6-trifluorobenzyl) (2-((2R,3S,4S,5S)-3,4,5-trihydroxy-6-(4-methoxyphenoxy)tetrahydro-2H-pyran-2-yl)ethyl)phosphinic acid (A54)

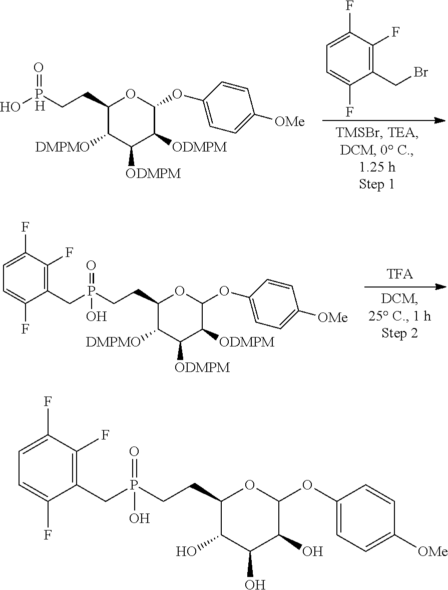

Step 1: A mixture of 2-[(2R,3R,4S,5S,6R)-3,4,5-tris[(3,4-dimethoxyphenyl)methoxy]-6-(4-methoxyphenoxy)tetrahydropyran-2-yl]ethylphosphinic acid (100 mg, 125 μmol), TMSBr (95.8 mg, 625 μmol) and TEA (63.3 mg, 625 μmol) in DCM (2 mL) was degassed and purged with N₂ for 3 times. Then the mixture was stirred at 0° C. for 15 min under N₂ atmosphere. Then 2-(bromomethyl)-1,3,4-trifluoro-benzene (56.3 mg, 250 μmol) was added. The mixture was stirred at 0° C. for 1 hour under N₂ atmosphere. LC-MS (ESI) found: 943 [M+H]⁺. The mixture was diluted with H₂O₁₀ mL, extracted with DCM (20 mL*3), dried over anhydrous Na₂SO₄, filtered and concentrated under reduced pressure. The residue was purified by prep-HPLC (Phenomenex C18 150*25 mm*10 μm; mobile phase: [water (NH₄HCO₃)−ACN]; gradient: 26%-56% B over 10 min) to afford (2,3,6-trifluorophenyl)methyl-[2-[(2R,3R,4S,5S)-3,4,5-tris[(3,4-dimethoxyphenyl)methoxy]-6-(4-methoxyphenoxy)tetrahydropyran-2-yl]ethyl]phosphinic acid (25 mg, 21.1% yield) as a white solid. LC-MS (ESI) found: 943 [M+H]⁺.

Step 2: To a solution of (2,3,6-trifluorophenyl)methyl-[2-[(2R,3R,4S,5S)-3,4,5-tris[(3,4-dimethoxyphenyl)methoxy]-6-(4-methoxyphenoxy)tetrahydropyran-2-yl]ethyl]phosphinic acid (25 mg, 26.5 μmol) in TFA (1 mL) and DCM (1 mL). The mixture was stirred at 25° C. for 1 hour. LC-MS (ESI) found: 493 [M+H]⁺. The mixture was concentrated under reduced pressure to give a residue. The residue was purified by prep-HPLC (Phenomenex luna C18 150*25 mm*10 μm; mobile phase: [water (TFA)−ACN]; gradient: 18%-48% B over 15 min) to afford (2,3,6-trifluorophenyl)methyl-[2-[(2R,3S,4S,5S)-3,4,5-trihydroxy-6-(4-methoxyphenoxy)tetrahydropyran-2-yl]ethyl]phosphinic acid (4.8 mg, 9.67 μmol, 99.2% purity) as a white solid. ¹H NMR (400 MHz, MeOD): δ 7.26-7.15 (m, 1H), 7.02-6.94 (m, 3H), 6.89-6.81 (m, 2H), 5.36 (d, J=1.6 Hz, 1H), 3.99 (dd, J=1.6, 3.2 Hz, 1H), 3.84 (dd, J=3.2, 9.2 Hz, 1H), 3.76 (s, 3H), 3.57-3.48 (m, 2H), 3.20 (d, J=16.4 Hz, 2H), 2.22-2.10 (m, 1H), 1.98-1.84 (m, 1H), 1.75-1.69 (m 1H), 1.57-1.42 (m, 1H) and LC-MS (ESI) found: 493 [M+H]⁺.

### Preparation of (2,4,6-trifluorobenzyl) (2-((2R,3S,4S,5S)-3,4,5-trihydroxy-6-(4-methoxyphenoxy)tetrahydro-2H-pyran-2-yl)ethyl)phosphinic acid (A55)

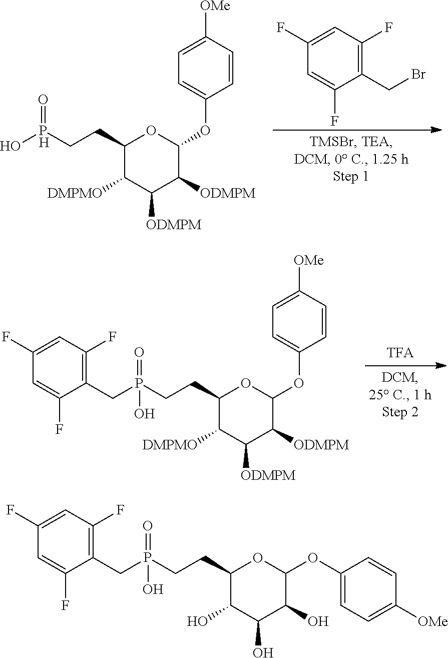

Step 1: A mixture of 2-[(2R,3R,4S,5S,6R)-3,4,5-tris[(3,4-dimethoxyphenyl)methoxy]-6-(4-methoxyphenoxy)tetrahydropyran-2-yl]ethylphosphinic acid (100 mg, 125 μmol), TMSBr (95.8 mg, 625 μmol) and TEA (63.3 mg, 625 μmol) in DCM (2 mL) was degassed and purged with N₂ for 3 times, then the mixture was stirred at 0° C. for 15 min under N₂ atmosphere. Then 2-(bromomethyl)-1,3,5-trifluoro-benzene (56.34 mg, 250 μmol) was added. The mixture was stirred at 0° C. for 1 hour under N₂ atmosphere. LC-MS (ESI) found: 943 [M+H]⁺. The mixture was diluted with H₂O₁₀ mL, extracted with DCM (20 mL*3), dried over anhydrous Na₂SO₄, filtered and concentrated under reduced pressure. The residue was purified by prep-HPLC (Phenomenex C18 150*25 mm*10 μm; mobile phase: [water (NH₄HCO₃)−ACN]; gradient: 26%-56% B over 10 min) to afford (2,4,6-trifluorophenyl)methyl-[2-[(2R,3R,4S,5S)-3,4,5-tris[(3,4-dimethoxyphenyl)methoxy]-6-(4-methoxyphenoxy)tetrahydropyran-2-yl]ethyl]phosphinic acid (45 mg, 38.1% yield) as a white solid. LC-MS (ESI) found: 943 [M+H]⁺.

Step 2: To a solution of (2,4,6-trifluorophenyl)methyl-[2-[(2R,3R,4S,5S)-3,4,5-tris[(3,4-dimethoxyphenyl)methoxy]-6-(4-methoxyphenoxy)tetrahydropyran-2-yl]ethyl]phosphinic acid (45 mg, 47.7 μmol) in TFA (1 mL) and DCM (1 mL). The mixture was stirred at 25° C. for 1 hour. LC-MS (ESI) found: 493 [M+H]⁺. The mixture was concentrated under reduced pressure to give a residue. The residue was purified by prep-HPLC (Phenomenex luna C18 150*25 mm*10 μm; mobile phase: [water (TFA)−ACN];gradient: 20%-50% B over 15 min) to afford (2,4,6-trifluorophenyl)methyl-[2-[(2R,3S,4S,5S)-3,4,5-trihydroxy-6-(4-methoxyphenoxy)tetrahydropyran-2-yl]ethyl]phosphinic acid (3 mg, 12.4% yield) as a white solid. ¹H NMR (400 MHz, MeOD): δ 7.03-6.94 (m, 2H), 6.90-6.79 (m, 4H), 5.37 (d, J=1.6 Hz, 1H), 3.99 (dd, J=1.6, 3.2 Hz, 1H), 3.83 (dd, J=3.4, 9.2 Hz, 1H), 3.76 (s, 3H), 3.54-3.47 (m, 2H), 3.11 (d, J=16.4 Hz, 2H), 2.20-2.06 (m, 1H), 1.95-1.79 (m, 1H), 1.77-1.64 (m, 1H), 1.52-1.36 (m, 1H) and LC-MS (ESI) found: 493 [M+H]⁺.

### Preparation of (cyclopropylmethyl) (2-((2R,3S,4S,5S)-3,4,5-trihydroxy-6-(4-methoxyphenoxy)tetrahydro-2H-pyran-2-yl)ethyl)phosphinic acid (A56)

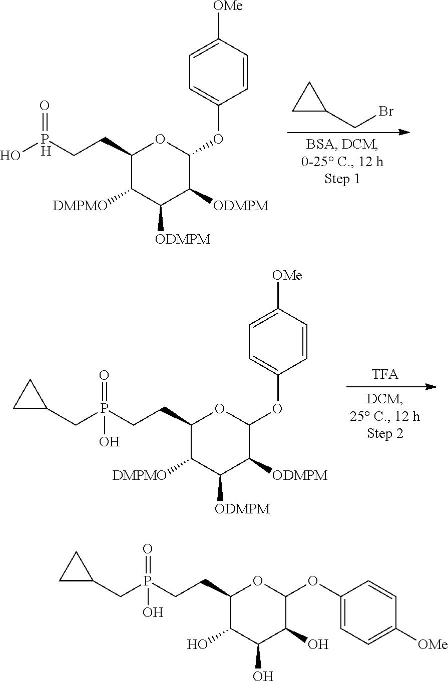

Step 1: A mixture of 2-[(2R,3R,4S,5S,6R)-3,4,5-tris[(3,4-dimethoxyphenyl)methoxy]-6-(4-methoxyphenoxy)tetrahydropyran-2-yl]ethylphosphinic acid (50 mg, 62.5 μmol), bromomethylcyclopropane (16.9 mg, 125 μmol) and BSA (89.1 mg, 438 μmol) in DCM (2 mL) was degassed and purged with N₂ for 3 times, and then the mixture was stirred at 0° C. for 12 hours under N₂ atmosphere. LC-MS (ESI) found: 853 [M+H]⁺. The mixture was diluted with H₂O₂₀ mL, extracted with DCM (30 mL*3), dried over anhydrous Na₂SO₄, filtered and concentrated under reduced pressure. The residue was purified by prep-HPLC (Welch Xtimate C18 150*25 mm*5 μm; mobile phase: [water (TFA)−ACN]; gradient: 42%-72% B over 10 min) to afford cyclopropylmethyl-[2-[(2R,3R,4S,5S)-3,4,5-tris[(3,4-dimethoxyphenyl)methoxy]-6-(4-methoxyphenoxy)tetrahydropyran-2-yl]ethyl]phosphinic acid (15 mg, 28.1% yield) as a white solid. ¹H NMR (400 MHz, CDCl₃): δ 6.94-6.75 (m, 13H), 5.36 (s, 1H), 4.91-4.58 (m, 6H), 4.05 (dd, J=2.4, 9.2 Hz, 1H), 3.93-3.84 (m, 10H), 3.79 (d, J=8.4 Hz, 9H), 3.76-3.70 (m, 4H), 3.65-3.57 (m, 1H), 2.16 (d, J=4.4 Hz, 1H), 1.93-1.56 (m, 6H), 0.48 (d, J=7.2 Hz, 2H), 0.09 (d, J=4.4 Hz, 2H) and LC-MS (ESI) found: 853 [M+H]⁺.

Step 2: A solution of cyclopropylmethyl-[2-[(2R,3R,4S,5S)-3,4,5-tris[(3,4-dimethoxyphenyl)methoxy]-6-(4-methoxyphenoxy)tetrahydropyran-2-yl]ethyl]phosphinic acid (13 mg, 15.2 μmol) in TFA (1 mL) and DCM (1 mL) was stirred at 25° C. for 1 hour. LC-MS (ESI) found: 403 [M+H]⁺. The mixture was concentrated under reduced pressure to give a residue. The residue was purified by prep-HPLC (Phenomenex luna C18 150*25 mm*10 μm; mobile phase: [water (TFA)−ACN]; gradient: 12%-42% B over 15 min) to afford cyclopropylmethyl-[2-[(2R,3S,4S,5S)-3,4,5-trihydroxy-6-(4-methoxyphenoxy)tetrahydropyran-2-yl]ethyl]phosphinic acid (1 mg, 15.6% yield) as a white solid. ¹H NMR (400 MHz, MeOD): δ 7.08-6.97 (m, 2H), 6.85 (d, J=9.2 Hz, 2H), 5.39 (d, J=1.6 Hz, 1H), 4.00 (dd, J=1.6, 3.4 Hz, 1H), 3.87-3.81 (m, 1H), 3.75 (s, 3H), 3.53 (d, J=6.4 Hz, 2H), 2.18-2.07 (m, 1H), 1.96-1.82 (m, 1H), 1.74-1.43 (m, 4H), 0.87-0.79 (m, 1H), 0.53 (d, J=7.6 Hz, 2H), 0.16 (d, J=3.6 Hz, 2H) and LC-MS (ESI) found: 403 [M+H]⁺.

### Preparation of propyl(2-((2R,3S,4S,5S)-3,4,5-trihydroxy-6-(4-methoxyphenoxy)tetrahydro-2H-pyran-2-yl)ethyl)phosphinic acid (A57)

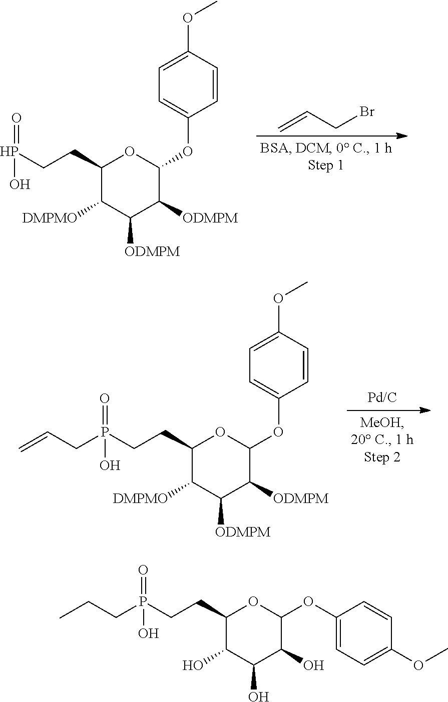

Step 1: A mixture of 2-[(2R,3R,4S,5S,6R)-3,4,5-tris[(3,4-dimethoxyphenyl)methoxy]-6-(4-methoxyphenoxy)tetrahydropyran-2-yl]ethylphosphinic acid (50 mg, 62.5 μmol), 3-bromoprop-1-ene (22.7 mg, 187 μmol) and BSA (89 mg, 438 μmol) in DCM (2 mL) was degassed and purged with N₂ for 3 times, and then the mixture was stirred at 0° C. for 1 hour under N₂ atmosphere. LC-MS (ESI) found: 839 [M+H]⁺. The mixture was concentrated under reduced pressure to give a residue. The residue was purified by prep-HPLC (Welch Xtimate C18 150*25 mm*5 μm; mobile phase: [water (TFA)−ACN]; gradient: 41%-71% B over 10 min) to afford allyl-[2-[(2R,3R,4S,5S)-3,4,5-tris[(3,4-dimethoxyphenyl)methoxy]-6-(4-methoxyphenoxy)tetrahydropyran-2-yl]ethyl]phosphinic acid (35 mg, 66.6% yield) as colorless oil. LC-MS (ESI) found: 853 [M+H]⁺.

Step 2: A mixture of allyl-[2-[(2R,3R,4S,5S)-3,4,5-tris[(3,4-dimethoxyphenyl)methoxy]-6-(4-methoxyphenoxy)tetrahydropyran-2-yl]ethyl]phosphinic acid (35 mg, 41.7 μmol) and Pd/C (22 mg, 10% purity) in MeOH (2 mL) was degassed and purged with H₂ (15 Psi) for 3 times, and then the mixture was stirred at 20° C. for 12 hours under H₂ (15 Psi) atmosphere. LC-MS (ESI) found: 391 [M+H]⁺. The mixture was filtered and concentrated under reduced pressure to give a residue. The residue was purified by prep-HPLC (Welch Xtimate C18 150*25 mm*5 μm; mobile phase: [water (TFA)−ACN]; gradient: 10%-40% B over 10 min) to afford propyl-[2-[(2R,3S,4S,5S)-3,4,5-trihydroxy-6-(4-methoxyphenoxy)tetrahydropyran-2-yl]ethyl]phosphinic acid (14 mg, 78.2% yield) as a white solid. ¹H NMR (400 MHz, MeOD): δ 7·O₇-6.94 (m, 2H), 6.91-6.81 (m, 2H), 5.39 (d, J=1.6 Hz, 1H), 4.00 (dd, J=1.6, 3.2 Hz, 1H), 3.88-3.82 (m, 1H), 3.75 (s, 3H), 3.55-3.49 (m, 2H), 2.14-2.02 (m, 1H), 1.90-1.75 (m, 1H), 1.71-1.42 (m, 6H), 1.05-0.97 (m, 3H) and LC-MS (ESI) found: 391 [M+H]⁺.

### Preparation of ((2,2-difluorocyclopropyl)methyl) (2-((2R,3S,4S,5S)-3,4,5-trihydroxy-6-(4-methoxyphenoxy)tetrahydro-2H-pyran-2-yl)ethyl)phosphinic acid (A58)

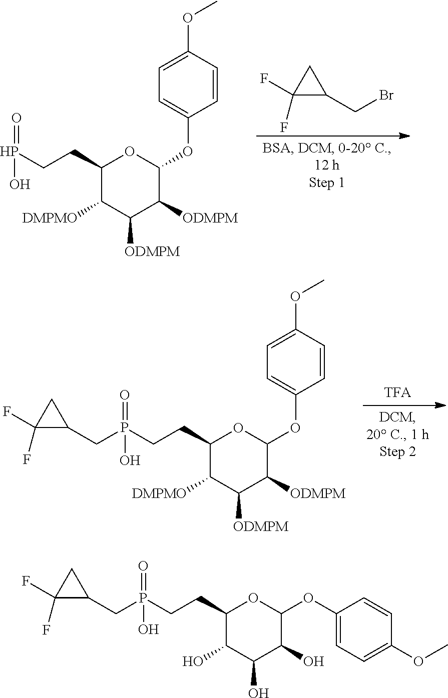

Step 1: A mixture of 2-[(2R,3R,4S,5S,6R)-3,4,5-tris[(3,4-dimethoxyphenyl)methoxy]-6-(4-methoxyphenoxy)tetrahydropyran-2-yl]ethylphosphinic acid (200 mg, 250 μmol), BSA (356 mg, 1.75 mmol) and 2-(bromomethyl)-1,1-difluoro-cyclopropane (85 mg, 500 μmol) in DCM (4 mL) was degassed and purged with N₂ for 3 times, and then the mixture was stirred at 0° C. for 1 hour under N₂ atmosphere. The mixture was stirred at 20° C. for 11 hours under N₂ atmosphere. LC-MS (ESI) found: 889 [M+H]⁺. The mixture was concentrated under reduced pressure to give a residue. The residue was purified by prep-HPLC (Phenomenex luna C18 150*25 mm*10 μm; mobile phase: [water (TFA)−ACN]; gradient: 42%-72% B over 10 min) to afford (2,2-difluorocyclopropyl)methyl-[2-[(2R,3R,4S,5S)-3,4,5-tris[(3,4-dimethoxyphenyl)methoxy]-6-(4-methoxyphenoxy)tetrahydropyran-2-yl]ethyl]phosphinic acid (40 mg, 17.9% yield) as a white solid. ¹H NMR (400 MHz, CDCl₃): δ 6.98-6.76 (m, 13H), 5.35 (s, 1H), 4.94-4.58 (m, 6H), 4.09-4.02 (m, 1H), 3.93-3.89 (m, 4H), 3.86 (d, J=10 Hz, 6H), 3.80 (s, 9H), 3.77-3.69 (m, 4H), 3.63 (d, J=8.4 Hz, 1H), 2.06 (dd, J=3.2, 6.4 Hz, 1H), 1.94-1.60 (m, 5H), 1.54-1.38 (m, 2H), 1.02 (d, J=6.4 Hz, 1H) and LC-MS (ESI) found: 889 [M+H]⁺.

Step 2: To a solution of (2,2-difluorocyclopropyl)methyl-[2-[(2R,3R,4S,5S)-3,4,5-tris[(3,4-dimethoxyphenyl)methoxy]-6-(4-methoxyphenoxy)tetrahydropyran-2-yl]ethyl]phosphinic acid (30 mg, 33.7 μmol) in DCM (1.5 mL) was added TFA (1.5 mL). The mixture was stirred at 20° C. for 1 hour. LC-MS (ESI) found: 439 [M+H]⁺. The mixture was concentrated under reduced pressure to give a residue. The residue was purified by prep-HPLC (Phenomenex Luna C18 150*25 mm*10 μm; mobile phase: [water (TFA)−ACN];gradient: 14%-44% B over 10 min) to afford (2,2-difluorocyclopropyl)methyl-[2-[(2R,3S,4S,5S)-3,4,5-trihydroxy-6-(4-methoxyphenoxy)tetrahydropyran-2-yl]ethyl]phosphinic acid (3.1 mg, 19.5% yield) as a white solid. ¹H NMR (400 MHz, MeOD): δ 7.00 (d, J=8.8 Hz, 2H), 6.84 (d, J=8.8 Hz, 2H), 5.37 (s, 1H), 4.01-3.95 (m, 1H), 3.84 (dd, J=3.2, 8.8 Hz, 1H), 3.74 (s, 3H), 3.57-3.47 (m, 2H), 2.16-2.04 (m, 1H), 1.96-1.81 (m, 2H), 1.76-1.56 (m, 3H), 1.55-1.41 (m, 2H), 1.15-1.01 (m, 1H) and LC-MS (ESI) found: 439 [M+H]⁺.

### Preparation of ((2,2-difluorocyclopropyl)methyl) (2-((2R,3S,4S,5S)-3,4,5-trihydroxy-6-(4-methoxyphenoxy)tetrahydro-2H-pyran-2-yl)ethyl)phosphinic acid (A59)

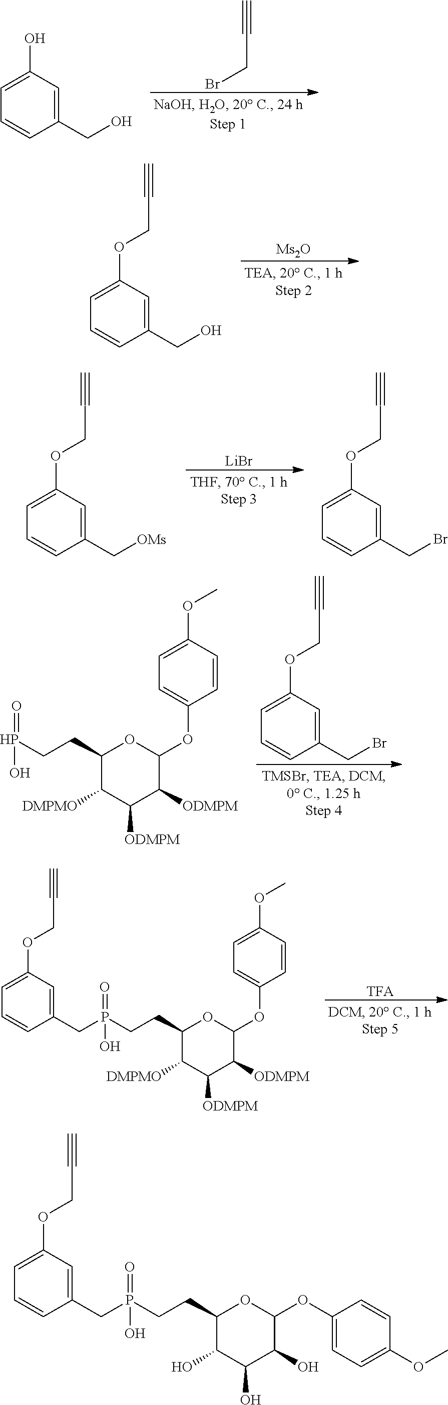

Step 1: To a solution of 3-(hydroxymethyl) phenol (2.00 g, 16.1 mmol) in H₂O (35 mL) was added NaOH (2.30 g, 57.5 mmol) and 3-bromoprop-1-yne (2.64 g, 17.7 mmol, 1.91 mL). The mixture was stirred at 20° C. for 24 hours. TLC showed 3-(hydroxymethyl) phenol was consumed completely. The mixture was extracted with EtOAc 300 mL (100 mL*3). The combined organic layers were washed with brine 100 mL, dried over anhydrous Na₂SO₄, filtered and concentrated under reduced pressure to give a residue. The residue was purified by column chromatography (SiO₂, petroleum ether:ethyl acetate=1:1, R_{f}=0.4) to afford (3-prop-2-ynoxyphenyl)methanol (720 mg, 27.5% yield) as yellow oil. ¹H NMR (400 MHz, CDCl₃): δ 7.36-7.23 (m, 1H), 7.05-6.97 (m, 2H), 6.92 (d, J=8.0 Hz, 1H), 4.95-4.42 (m, 4H), 2.53 (s, 1H), 1.70 (s, 1H) Step 2: To a solution of (3-prop-2-ynoxyphenyl)methanol (720 mg, 4.44 mmol) in DCM (10 mL) was added TEA (673 mg, 6.66 mmol) and methylsulfonyl methanesulfonate (850 mg, 4.88 mmol). The mixture was stirred at 20° C. for 1 hour. TLC (petroleum ether:ethyl acetate=1:1) showed (3-prop-2-ynoxyphenyl)methanol was consumed completely. The mixture was quenched with 1 N HCl (20 mL) and extracted with DCM mL (20 mL*3). The combined organic layers were washed with brine 20 mL, dried over anhydrous Na₂SO₄, filtered and concentrated under reduced pressure to afford (3-prop-2-ynoxyphenyl)methyl methanesulfonate (800 mg, crude) as yellow oil.

Step 3: To a solution of (3-prop-2-ynoxyphenyl)methyl methanesulfonate (700 mg, 2.91 mmol) in THF (10 mL) was added LiBr (632 mg, 7.28 mmol). The mixture was stirred at 70° C. for 1 hour. TLC (petroleum ether:ethyl acetate=10:1) showed (3-prop-2-ynoxyphenyl)methyl methanesulfonate was consumed completely. The mixture was diluted with H₂O₂₀ mL and extracted with EtOAc 60 mL (20 mL*3). The combined organic layers were washed with 1 N HCl 20 mL and brine 20 mL, dried over anhydrous Na₂SO₄, filtered and concentrated under reduced pressure to afford 1-(bromomethyl)-3-prop-2-ynoxy-benzene (500 mg, 76.2% yield) as yellow oil. ¹H NMR (400 MHz, CDCl₃): δ 7.33-7.26 (m, 1H), 7.07-7.00 (m, 2H), 6.96-6.90 (m, 1H), 4.71 (d, J=2.4 Hz, 2H), 4.48 (s, 2H), 2.55 (t, J=2.4 Hz, 1H)

Step 4: A mixture of 2-[(2R,3R,4S,5S,6R)-3,4,5-tris[(3,4-dimethoxyphenyl)methoxy]-6-(4-methoxyphenoxy)tetrahydropyran-2-yl]ethylphosphinic acid (100 mg, 125 μmol), TEA (63.3 mg, 625 μmol) and TMSBr (95.8 mg, 625 μmol) in DCM (1 mL) was degassed and purged with N₂ for 3 times, Then the mixture was stirred at 0° C. for 15 min under N₂ atmosphere. Then 1-(bromomethyl)-3-prop-2-ynoxy-benzene (56.3 mg, 250 μmol) was added. The mixture was stirred at 0° C. for 1 h under N₂ atmosphere. LC-MS (ESI) found: 943 [M+H]⁺. The mixture was adjusted by 1 N NaOH to pH=7. The mixture was concentrated under reduced pressure to give a residue. The residue was purified by prep-HPLC (TFA condition) to afford (3-prop-2-ynoxyphenyl)methyl-[2-[(2R,3R,4S,5S)-3,4,5-tris[(3,4-dimethoxyphenyl)methoxy]-6-(4-methoxyphenoxy)tetrahydropyran-2-yl]ethyl]phosphinic acid (30 mg, 25.4% yield) as colourless oil.

Step 5: To a solution of (3-prop-2-ynoxyphenyl)methyl-[2-[(2R,3R,4S,5S)-3,4,5-tris[(3,4-dimethoxyphenyl)methoxy]-6-(4-methoxyphenoxy)tetrahydropyran-2-yl]ethyl]phosphinic acid (20 mg, 21.2 μmol) in DCM (0.9 mL) and TFA (0.1 mL). The mixture was stirred at 20° C. for 1 hour. LC-MS (ESI) found: 493 [M+H]⁺. The mixture was concentrated under reduced pressure to give a residue. The residue was purified by prep-HPLC (Phenomenex luna C18 150*25 mm*10 μm; mobile phase: [water (TFA)−ACN]; gradient: 20%-50% B over 10 min) to afford (3-prop-2-ynoxyphenyl)methyl-[2-[(2R,3S,4S,5S)-3,4,5-trihydroxy-6-(4-methoxyphenoxy)tetrahydropyran-2-yl]ethyl]phosphinic acid (2.8 mg, 26.0% yield) as a white solid. ¹H NMR (400 MHz, MeOD): δ 7.22 (t, J=8.0 Hz, 1H), 6.97-6.80 (m, 7H), 5.35 (d, J=1.6 Hz, 1H), 4.71 (d, J=2.4 Hz, 2H), 3.98 (dd, J=1.6, 3.2 Hz, 1H), 3.81 (dd, J=3.2, 8.8 Hz, 1H), 3.75 (s, 3H), 3.56-3.43 (m, 2H), 3.13-3.03 (m, 2H), 2.92 (t, J=2.4 Hz, 1H), 2.16-2.05 (m, 1H), 1.83-1.63 (m, 2H), 1.46-1.28 (m, 1H) and LC-MS (ESI) found: 493 [M+H]⁺.

### Preparation of (2,6-difluorobenzyl) (2-((2R,3S,4S,5S)-3,4,5,6-tetrahydroxytetrahydro-2H-pyran-2-yl)ethyl)phosphinic acid (A60)

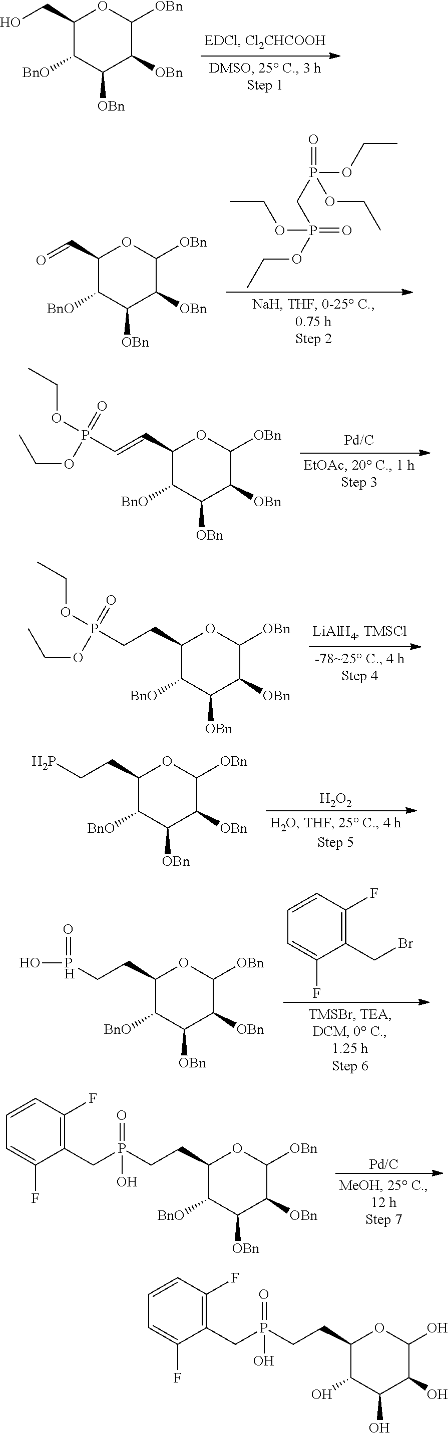

Step 1: To a solution of [(2R,3R,4S,5S,6S)-3,4,5,6-tetrabenzyloxytetrahydropyran-2-yl]methanol (10.0 g, 18.5 mmol) in DMSO (100 mL) was added EDCI (12.0 g, 62.8 mmol) and 2,2-dichloroacetic acid (1.19 g, 9.25 mmol). The mixture was stirred at 25° C. for 3 hours. TLC (petroleum ether:ethyl acetate=3:1) showed [(2R,3R,4S,5S,6S)-3,4,5,6-tetrabenzyloxytetrahydropyran-2-yl]methanol was consumed completely. The reaction mixture was quenched by H₂O₂₀₀ mL, and extracted with EtOAc (200 mL*3). The combined organic layers were washed with brine (100 mL*3), dried over anhydrous Na₂SO₄, filtered and concentrated under reduced pressure to afford (2S,3S,4S,5S)-3,4,5,6-tetrabenzyloxytetrahydropyran-2-carbaldehyde (10 g, crude) as colorless oil.

Step 2: To a solution of 1-[diethoxyphosphorylmethyl(ethoxy)phosphoryl]oxyethane (5.89 g, 20.4 mmol) in THF (50 mL) was added NaH (965 mg, 24.1 mmol, 60% purity) under 0° C. The mixture was stirred at 0° C. for 15 min. Then (2S,3S,4S,5S)-3,4,5,6-tetrabenzyloxytetrahydropyran-2-carbaldehyde (10.0 g, 18.5 mmol) in THF (50 mL) was added. The mixture was stirred at 0° C. for 0.5 hour. LC-MS (ESI) found: 673 [M+H]⁺. TLC showed the desired spot was detected. The mixture was quenched by addition saturated NH₄Cl aqueous solution (200 mL) at 0° C., extracted with EtOAc (200 mL*3), the combined organic layers were washed with brine (200 mL), dried over anhydrous Na₂SO₄, filtered and concentrated under reduced pressure. The residue was purified by column chromatography (SiO₂, petroleum ether:ethyl acetate=1:1, R_{f}=0.2) to afford (3S,4S,5R,6R)-2,3,4,5-tetrabenzyloxy-6-[(E)-2-diethoxyphosphorylvinyl]tetrahydropyran (5.00 g, 40.0% yield) as colorless oil. LC-MS (ESI) found: 673 [M+H]⁺.

Step 3: A mixture of (3S,4S,5R,6R)-2,3,4,5-tetrabenzyloxy-6-[(E)-2-diethoxyphosphorylvinyl]tetrahydropyran (2.00 g, 2.97 mmol) and Pd/C (632 mg, 10% purity) in EtOAc (20 mL) was degassed and purged with H₂ (15 Psi) for 3 times, and then the mixture was stirred at 25° C. for 1 hour under H₂ (15 Psi) atmosphere. LC-MS (ESI) found: 675 [M+H], The mixture was filtered and concentrated under reduced pressure to afford compound (3S,4S,5R,6R)-2,3,4,5-tetrabenzyloxy-6-(2-diethoxyphosphorylethyl)tetrahydropyran (1.90 g, 94.7% yield) as yellow oil. LC-MS (ESI) found: 675 [M+H]⁺.

Step 4: To a solution of LAH (1.60 g, 42.2 mmol) in THF (25 mL) was degassed and purged with N₂ for 3 times, then TMSCl (3.67 g, 33.7 mmol) was added under −78° C. and the mixture was stirred at 25° C. for 1 hour under N₂ atmosphere. Then (3S,4S,5R,6R)-2,3,4,5-tetrabenzyloxy-6-(2-diethoxyphosphorylethyl)tetrahydropyran (1.90 g, 2.82 mmol) in THF (25 mL) was added under 0° C. The mixture was stirred at 25° C. for 3 hours under N₂ atmosphere. LC-MS (ESI) found: 593 [M+Na]⁺. TLC showed (3S,4S,5R,6R)-2,3,4,5-tetrabenzyloxy-6-(2-diethoxyphosphorylethyl)tetrahydropyran was consumed completely. The reaction was quenched with H₂O (50 mL) at 0° C., and then diluted with EtOAc 100 mL and filtered. The mixture was extracted with EtOAc 600 mL (200 mL*3). The combined organic layers were washed with brine 200 mL, dried over anhydrous Na₂SO₄, filtered and concentrated under reduced pressure to give a residue. The residue was purified by column chromatography (SiO₂, petroleum ether:ethyl acetate=10:1, R_{f}=0.5) to afford 2-[(2R,3R,4S,5S)-3,4,5,6-tetrabenzyloxytetrahydropyran-2-yl]ethylphosphane (1.00 g, 62.2% yield) as colorless oil. LC-MS (ESI) found: 593 [M+Na]⁺.

Step 5: To a solution of 2-[(2R,3R,4S,5S)-3,4,5,6-tetrabenzyloxytetrahydropyran-2-yl]ethylphosphane (1.00 g, 1.76 mmol) in THF (5 mL) and H₂O (5 mL) was added H₂O₂ (1.36 g, 12.0 mmol, 30% purity). The mixture was stirred at 20° C. for 4 hours. LC-MS (ESI) found: 603 [M+H]⁺. The reaction mixture was quenched by addition saturated NaHSO₃ aqueous solution 20 mL at 0° C., and extracted with EtOAc (100 mL*3). The combined organic layers were washed with addition saturated NaHSO₃ aqueous solution (50 mL*3), dried over anhydrous Na₂SO₄, filtered and concentrated under reduced pressure to give a residue. The residue was purified by prep-HPLC (Phenomenex C18 250*50 mm *10 μm; mobile phase: [water (NH₄HCO₃)−ACN]; gradient: 33%-63% B over 10 min) to afford 2-[(2R,3R,4S,5S)-3,4,5,6-tetrabenzyloxytetrahydropyran-2-yl]ethylphosphinic acid (220 mg, 365 μmol) as colorless oil. LC-MS (ESI) found: 603 [M+H]⁺.

Step 6: A mixture of 2-[(2R,3R,4S,5S)-3,4,5,6-tetrabenzyloxytetrahydropyran-2-yl]ethylphosphinic acid (200 mg, 331 μmol), TEA (167 mg, 1.66 mmol) and TMSBr (254 mg, 1.66 mmol) in DCM (2 mL) was degassed and purged with N₂ for 3 times, then the mixture was stirred at 0° C. for 15 min under N₂ atmosphere. Then 2-(bromomethyl)-1,3-difluoro-benzene (137 mg, 663 μmol) was added. The mixture was stirred at 0° C. for 1 hour under N₂ atmosphere. LC-MS (ESI) found: 729 [M+H]⁺. The reaction mixture was diluted with H₂O₁₀ mL and extracted with DCM 60 mL (20 mL*3). The combined organic layers were dried over anhydrous Na₂SO₄, filtered and concentrated under reduced pressure to give a residue. The residue was combined with the first batch (EC8492-320-P1), then purified by prep-HPLC (Welch Xtimate C18 150*25 mm*5 μm; mobile phase: [water (TFA)−ACN]; gradient: 55%-85% B over 10 min) to afford (2,6-difluorophenyl)methyl-[2-[(2R,3R,4S,5S)-3,4,5,6-tetrabenzyloxytetrahydropyran-2-yl]ethyl]phosphinic acid (100 mg, 41.3% yield) as yellow oil. ¹H NMR (400 MHz, CDCl₃): δ 7.40 -7.27 (m, 17H), 7.26-7.21 (m, 3H), 7.14-7.07 (m, 1H), 6.81 (t, J=7.6 Hz, 2H), 4.94-4.54 (m, 8H), 4.37 (d, J=12.0 Hz, 1H), 3.92 (dd, J=2.8, 8.8 Hz, 1H), 3.80 (d, J=1.6 Hz, 1H), 3.70 (t, J=9.2 Hz, 1H), 3.65-3.57 (m, 1H), 3.15 (d, J=16.4 Hz, 2H), 2.28-2.14 (m, 1H), 2.08-1.93 (m, 1H), 1.90-1.78 (m, 1H), 1.75-1.59 (m, 1H) and LC-MS (ESI) found: 729 [M+H]⁺. Step 7: A mixture of (2,6-difluorophenyl)methyl-[2-[(2R,3R,4S,5S)-3,4,5,6-tetrabenzyloxytetrahydropyran-2-yl]ethyl]phosphinic acid (20 mg, 27.4 μmol) and Pd/C (14.6 mg, 10% purity) in MeOH (2 mL) was degassed and purged with H₂ (15 Psi) for 3 times, then the mixture was stirred at 20° C. for 12 hours under H₂ (15 Psi) atmosphere. LC-MS (ESI) found: 369 [M+H]⁺. The mixture was filtered and concentrated under reduced pressure to afford compound (2,6-difluorophenyl)methyl-[2-[(2R,3S,4S,5S)-3,4,5,6-tetrahydroxytetrahydropyran-2-yl]ethyl]phosphinic acid (2 mg, 19.2% yield) as a white solid. ¹H NMR (400 MHz, MeOD): δ 7.27 -7.14 (m, 1H), 6.96-6.86 (m, 2H), 5.02 (s, 1H), 3.84-3.63 (m, 3H), 3.48-3.40 (m, 1H), 3.04 (d, J=16.4 Hz, 2H), 2.25-2.08 (m, 1H), 1.93-1.68 (m, 2H), 1.64-1.52 (m, 1H) and LC-MS (ESI) found: 369 [M+H]⁺.

### Preparation of (2-((2R,3R,4R,5R)-1-acetyl-3,4,5-trihydroxypiperidin-2-yl)ethyl)phosphonic acid (A61)

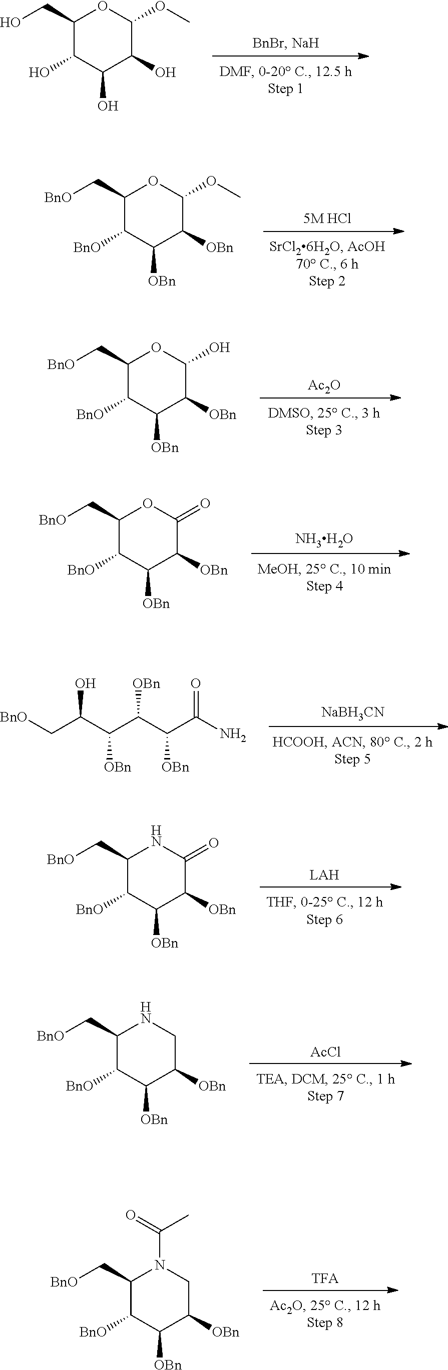

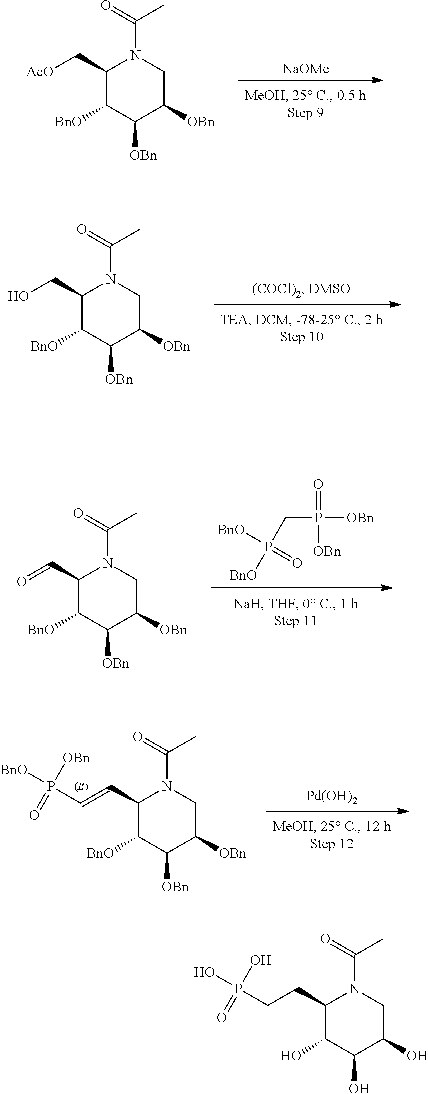

Step 1: To a stirred solution of (2R,3S,4S,5S,6S)-2-(hydroxymethyl)-6-methoxy-tetrahydropyran-3,4,5-triol (100 g, 514 mmol) in THF (500 mL) was added NaH (123.58 g, 3.09 mol, 60% purity) in portions at 0° C. After addition, the reaction mixture was stirred at this temperature for 30 min. BnBr (528 g, 3.09 mol) was added dropwise to the reaction mixture. After addition, the reaction mixture was stirred at 20° C. for 12 hours. LC-MS (ESI) found: 577 [M+Na]⁺. Under N₂ atmosphere, the reaction was quenched with ice sat. NH₄Cl (12 L). The residue was extracted with CH₂Cl₂ (1000 mL*2), and the combined organic layers were washed with water (1000 mL*3) and brine 1000 mL (500 mL*2), dried over anhydrous Na₂SO₄, filtered and concentrated under reduced pressure to give a residue. The residue was purified by column chromatography (SiO₂, Petroleum ether: Ethyl acetate=5:1) to afford (2R,3R,4S,5S,6S)-3,4,5-tribenzyloxy-2-(benzyloxymethyl)-6-methoxy-tetrahydropyran (158 g, 49.23% yield) as yellow oil. LC-MS (ESI) found: 577 [M+Na]⁺

Step 2: A solution of (2R,3R,4S,5S,6S)-3,4,5-tribenzyloxy-2-(benzyloxymethyl)-6-methoxy-tetrahydropyran (83.1 g, 133 mmol) in acetic acid (444 mL) was stirred and heated to 70° C. Then to the mixture was added HCl (5 M, 74 mL), followed by the addition of dichlorostrontium (2.11 g, 13.3 mmol). The mixture was stirred at 70° C. for 6 hours. LC-MS (ESI) found: 563 [M+Na]⁺. The reaction mixture was diluted with ice-water 1000 mL and extracted with DCM 500 mL (250 mL*2). The combined organic layers were washed with brine 100 mL (50 mL*2), dried over anhydrous Na₂SO₄, filtered and concentrated under reduced pressure to give a residue. The residue was purified by reversed-phase HPLC (TFA condition) to afford (2S,3S,4S,5R,6R)-3,4,5-tribenzyloxy-6-(benzyloxymethyl)tetrahydropyran-2-ol (53 g, 70.54% yield) as yellow oil. 1H NMR (400 MHz, CDCl₃): δ 7.35-7.29 (m, 18H), 7.18 (s, 2H), 5.27 (s, 3H), 4.90-4.92 (m, 8H), 4.13-4.12 (m, 1H), 3.96-3.82 (m, 3H), 3.74-3.32 (m, 3H) and LC-MS (ESI) found: 563 [M+Na]⁺.

Step 3: To a solution of (2S,3S,4S,5R,6R)-3,4,5-tribenzyloxy-6-(benzyloxymethyl)tetrahydropyran-2-ol (32 g, 56.8 mmol) in DMSO (320 mL) was added acetic anhydride (112 mL). The mixture was stirred at 25° C. for 3 hours. LC-MS (ESI) found: 561 [M+Na]⁺. The reaction mixture was diluted with ice H₂O₁₀₀₀ mL and extracted with MTBE 2000 mL (500 mL*4). The combined organic layers were washed with brine 1000 mL (500 mL*2), dried over anhydrous Na₂SO₄, filtered and concentrated under reduced pressure to afford (3S,4S,5R,6R)-3,4,5-tribenzyloxy-6-(benzyloxymethyl)tetrahydropyran-2-one (33 g, crude) as a yellow solid. LC-MS (ESI) found: 561 [M+Na]⁺.

Step 4: A mixture of (3S,4S,5R,6R)-3,4,5-tribenzyloxy-6-(benzyloxymethyl)tetrahydropyran-2-one (21 g, 38.9 mmol) in NH₃/MeOH (7 M, 5.57 mL) was degassed and purged with N₂ for 3 times, and then the mixture was stirred at 25° C. for 10 min under N₂ atmosphere. LC-MS (ESI) found: 556 [M+H]⁺. The reaction mixture was concentrated under reduced pressure to afford (2R,3S,4R,5R)-2,3,4,6-tetrabenzyloxy-5-hydroxy-hexanamide (18 g, crude) as a white solid. LC-MS (ESI) found: 556 [M+H]⁺.

Step 5: To a solution of (2R,3S,4S)-2,3,4,6-tetrabenzyloxy-5-oxo-hexanamide (20.0 g, 36.1 mmol) in ACN (150 mL) and HCOOH (30 mL) was added NaBH₃CN (4.54 g, 72.2 mmol). The mixture was stirred at 80° C. for 2 hours. LC-MS (ESI) found: 538 [M+H]⁺. The mixture was then cooled by ice and the reaction was quenched with 0.1 M HCl 200 mL. After stirring for 15 minutes, the mixture was poured into a mixture of ethyl acetate and saturated aqueous NaHCO₃ solution (1/1, v/v, 1 L). The water layer was separated and extracted with ethyl acetate (500 ml*3), the combined organic layers were washed with brine 300 mL and dried over anhydrous Na₂SO₄, filtered and concentrated under reduced pressure to give a residue. The residue was purified by prep-HPLC (Phenomenex luna C18 (250 * 70 mm, 10 μm); mobile phase: [water (TFA)−ACN]; B %: 45%-85%, 23 min) to afford (3S,4S,5R,6R)-3,4,5-tribenzyloxy-6-(benzyloxymethyl) piperidin-2-one (6.40 g, 32.9% yield) as red oil. ¹H NMR (400 MHz, CDCl₃): δ 7.47-6.98 (m, 20H), 5.20-5.00 (m, 1H), 4.93-4.19 (m, 9H), 4.11-3.78 (m, 2H), 3.76-3.29 (m, 3H) and LC-MS (ESI) found: 538 [M+H]Step 6: A mixture of (3S,4S,5R,6R)-3,4,5-tribenzyloxy-6-(benzyloxymethyl) piperidin-2-one (2.00 g, 3.72 mmol) in THF (20 mL) was degassed and purged with N₂ for 3 times, then LAH (395 mg, 10.4 mmol) was added under 0° C., and then the mixture was stirred at 25° C. for 12 hours under N₂ atmosphere. LC-MS (ESI) found: 524 [M+H]⁺. The reaction was quenched with H₂O (50 mL) at 0° C., then diluted with EtOAc 50 mL and filtered. The mixture was extracted with EtOAc 150 mL (50 mL*3). The combined organic layers were washed with brine 50 mL, dried over Na₂SO₄, filtered and concentrated under reduced pressure to give a residue. The residue was purified by prep-HPLC (TFA condition) to afford (2R,3R,4R,5R)-3,4,5-tribenzyloxy-2-(benzyloxymethyl) piperidine (1.00 g, 51.3% yield) yellow oil. LC-MS (ESI) found: 524 [M+H]⁺.

Step 7: To a solution of (2R,3R,4R,5R)-3,4,5-tribenzyloxy-2-(benzyloxymethyl) piperidine (1.00 g, 1.91 mmol) in DCM (10 mL) was added TEA (386 mg, 3.82 mmol) and acetyl chloride (299 mg, 3.82 mmol). The mixture was stirred at 25° C. for 1 hour. LC-MS (ESI) found: 566 [M+H]⁺. The mixture was diluted with H₂O₃₀ mL and extracted with DCM 90 mL (30 mL*3). The combined organic layers were washed with 0.5N HCl 20 mL and brine 30 mL, dried over anhydrous Na₂SO₄, filtered and concentrated under reduced pressure to afford compound 1-[(2R,3R,4R,5R)-3,4,5-tribenzyloxy-2-(benzyloxymethyl)-1-piperidyl]ethanone (1.10 g, crude) as yellow oil. LC-MS (ESI) found: 566 [M+H]⁺.

Step 8: A solution of 1-[(2R,3R,4R,5R)-3,4,5-tribenzyloxy-2-(benzyloxymethyl)-1-piperidyl]ethanone (1.10 g, 1.94 mmol) in TFA (2 mL) and Ac₂₀ (8 mL) was stirred at 25° C. for 12 hours. LC-MS (ESI) found: 518 [M+H]⁺. The mixture was adjusted by saturated aqueous sodium bicarbonate to pH=7, and the reaction mixture was then extracted with ethyl acetate (50 mL*3) and washed with brine 50 mL. The organic layer was dried over anhydrous Na₂SO₄ and the filtrate was concentrated in a vacuum evaporator. The residue was purified by prep-HPLC (Phenomenex Luna C18 200*40 mm*10 μm; mobile phase: [water (TFA)−ACN]; B %: 53%-83%, 10 min) to afford [(2R,3R,4R,5R)-1-acetyl-3,4,5-tribenzyloxy-2-piperidyl]methyl acetate (0.55 g, 54.6% yield) as yellow oil. LC-MS (ESI) found: 518 [M+H]⁺.

Step 9: To a solution of [(2R,3R,4R,5R)-1-acetyl-3,4,5-tribenzyloxy-2-piperidyl]methyl acetate (550 mg, 1.06 mmol) in MeOH (10 mL) was added NaOMe (28.7 mg, 531 μmol). The mixture was stirred at 25° C. for 0.5 hour. LC-MS (ESI) found: 476 [M+H]⁺. Saturated aqueous sodium bicarbonate (5 mL) was added to the reaction mixture, and the reaction mixture was then extracted with ethyl acetate (20 mL*3) and washed with brine 20 mL. The organic layers were dried over anhydrous Na₂SO₄ and the filtrate was concentrated in a vacuum evaporator to afford 1-[(2R,3R,4R,5R)-3,4,5-tribenzyloxy-2-(hydroxymethyl)-1-piperidyl]ethanone (0.50 g, crude) as yellow oil. LC-MS (ESI) found: 476 [M+H]⁺.

Step 10: To a solution of DMSO (49.2 mg, 630 μmol) in DCM (2 mL), the mixture was cooled to −75° C. Then (COCl)₂ (53.3 mg, 420 μmol) was added, and 1-[(2R,3R,4R,5R)-3,4,5-tribenzyloxy-2-(hydroxymethyl)-1-piperidyl]ethanone (100 mg, 210 μmol) in DCM (2 mL) was added. The mixture was stirred at −75° C. for 1 hour. Then TEA (170 mg, 1.68 mmol) was added at −75° C. The mixture was stirred at 25° C. for 1 hour, LC-MS (ESI) found: 474 [M+H]⁺. The reaction mixture was quenched by addition H₂O₁₀ mL at 20° C., and extracted with DCM 30 mL (10 mL*3). The combined organic layers were washed with brine 10 mL, dried over anhydrous Na₂SO₄, filtered and concentrated under reduced pressure to afford compound (2S,3R,4R,5R)-1-acetyl-3,4,5-tribenzyloxy-piperidine-2-carbaldehyde (0.10 g, crude) as yellow oil. LC-MS (ESI) found: 474 [M+H]⁺.

Step 11: To a solution of tetrabenzyl methylenebis(phosphonate) (169 mg, 316 μmol) in THF (2 mL) was added NaH (12.67 mg, 316 μmol, 60% purity) under 0° C. The mixture was stirred at 0° C. for 15 min. Then (2S,3R,4R,5R)-1-acetyl-3,4,5-tribenzyloxy-piperidine-2-carbaldehyde (100 mg, 211 μmol) in THF (2 mL) was added. The mixture was stirred at 25° C. for 0.5 hour. LC-MS (ESI) found: 732 [M+H]⁺. The mixture was quenched by addition saturated NH₄Cl aqueous solution (10 mL) at 0° C., extracted with EtOAc (20 mL*3), the combined organic layers were washed with brine (10 mL), dried over anhydrous Na₂SO₄, filtered and concentrated under reduced pressure. The residue was purified by prep-HPLC (Phenomenex luna C18 150*25 mm*10 μm; mobile phase: [water (TFA)−ACN]; gradient: 66%-96% B over 15 min) to afford 1-[(2R,3R,4R,5R)-3,4,5-tribenzyloxy-2-[(E)-2-dibenzyloxyphosphorylvinyl]-1-piperidyl]ethanone (30 mg, crude) as yellow oil. LC-MS (ESI) found: 732 [M+H]⁺.

Step 12 A mixture of 1-[(2R,3R,4R,5R)-3,4,5-tribenzyloxy-2-[(E)-2-dibenzyloxyphosphorylvinyl]-1-piperidyl]ethanone (20 mg, 27.3 μmol) and Pd(OH)₂/C (19.1 mg, 20% purity) in MeOH (2 mL) was degassed and purged with H₂ (15 Psi) for 3 times, and then the mixture was stirred at 25° C. for 12 hours under H₂ (15 Psi) atmosphere. LC-MS (ESI) found: 284 [M+H]⁺. The mixture was filtered and concentrated under reduced pressure to afford 2-[(2R,3R,4R,5R)-1-acetyl-3,4,5-trihydroxy-2-piperidyl]ethylphosphonic acid (4 mg, 51.68% yield) as a white solid. LC-MS (ESI) found: 284 [M+H]⁺.

### Preparation of (2-((2R,3S,4S,5S,6R)-6-((7-(hex-5-ynamido)-9H-fluoren-2-yl)oxy)-3,4,5-trihydroxytetrahydro-2H-pyran-2-yl)ethyl)phosphonic acid (A62)

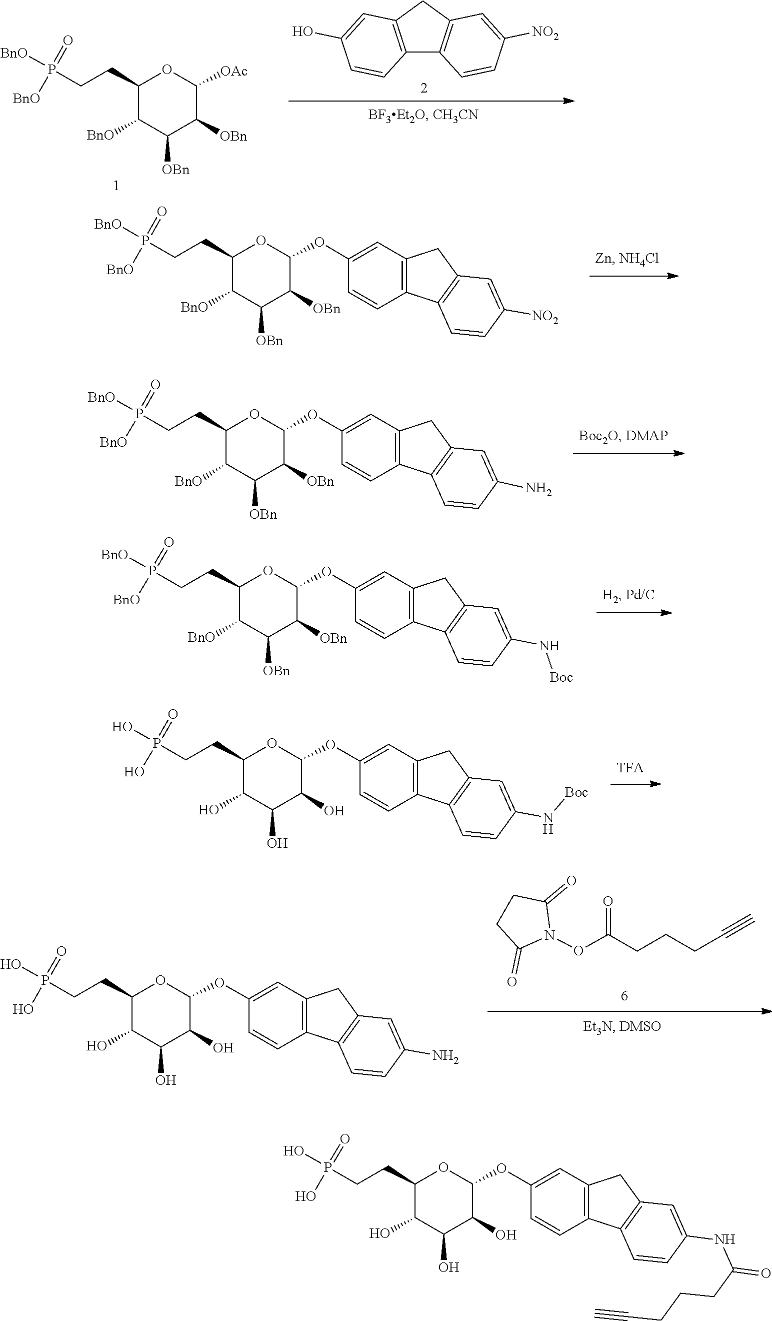

### Preparation of (2-((2R,3S,4S,5S,6R)-6-((6-(hex-5-ynamido) biphenylen-2-yl)oxy)-3,4,5-trihydroxytetrahydro-2H-pyran-2-yl)ethyl)phosphonic acid (A63)

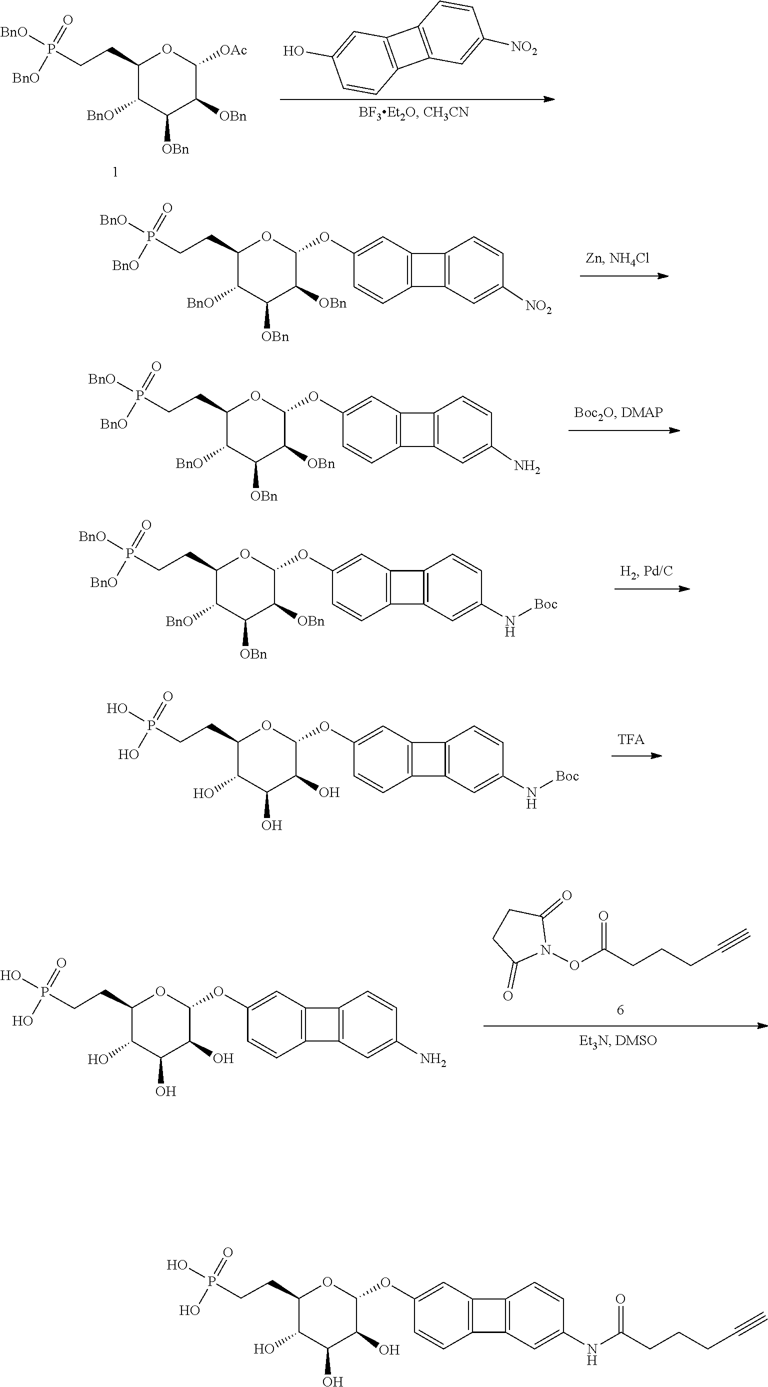

### Preparation of (2-((2R,3S,4S,5S,6R)-3,4,5-trihydroxy-6-((2-(pent-4-ynamido)-6,7-dihydrodibenzo[b,d]oxepin-9-yl)oxy)tetrahydro-2H-pyran-2-yl)ethyl)phosphonic acid (A64)

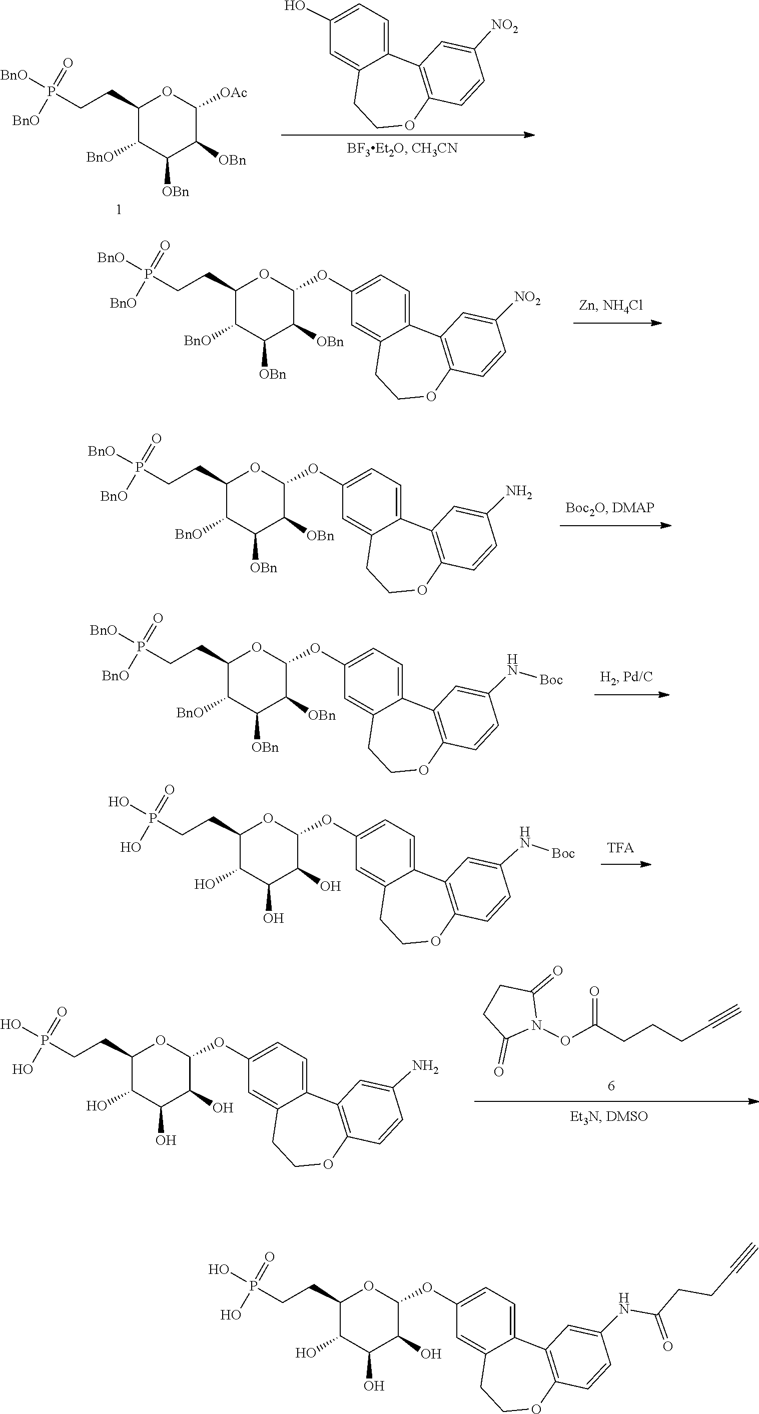

### Preparation of (2-((2R,3S,4S,5S,6R)-6-((7-(hex-5-ynamido)-10,11-dihydrodibenzo[b,f]oxepin-3-yl)oxy)-3,4,5-trihydroxytetrahydro-2H-pyran-2-yl)ethyl)phosphonic acid (A65)

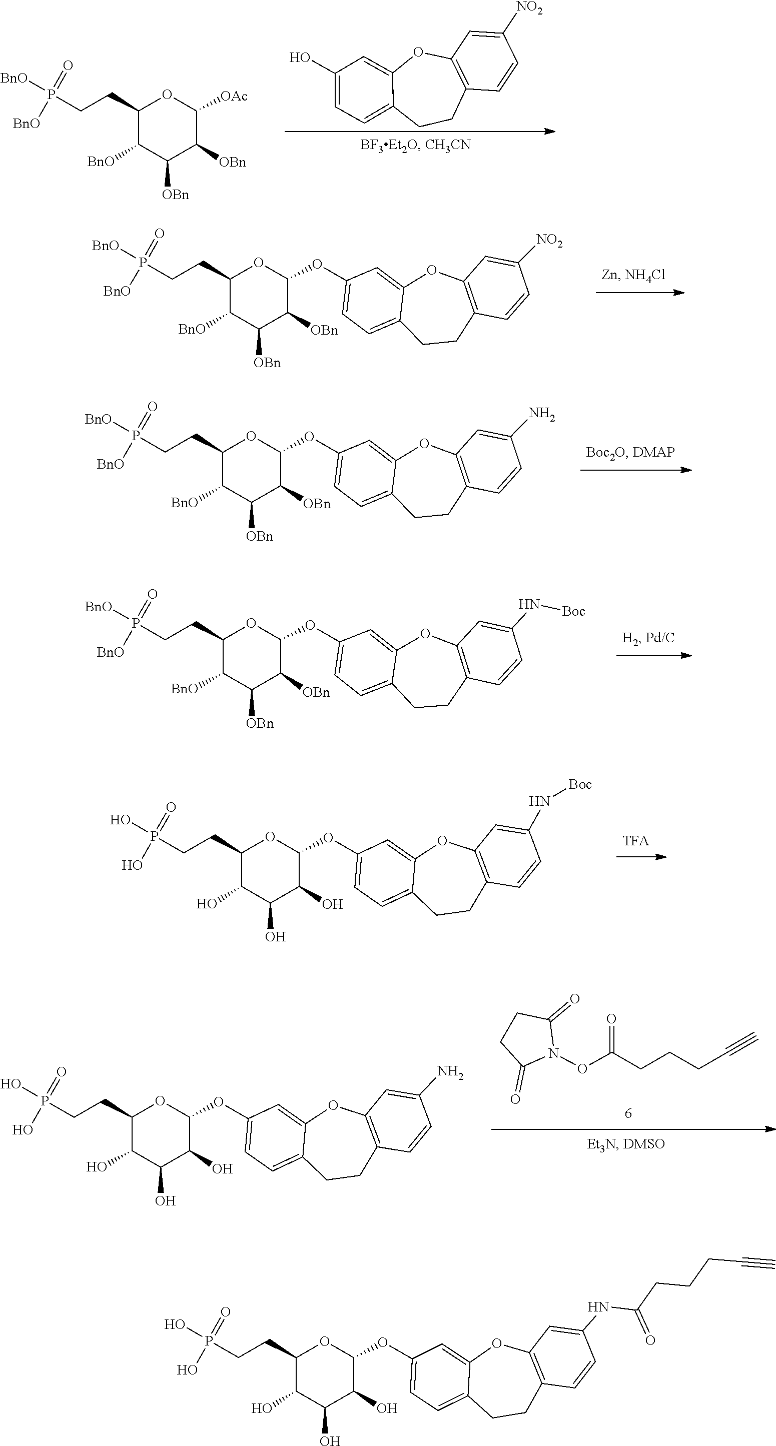

### Preparation of (2-((2R,3R,4R,5R)-1-(3-(4-(hex-5-ynamido)phenyl)propanoyl)-3,4,5-trihydroxypiperidin-2-yl)ethyl)phosphonic acid (A66)

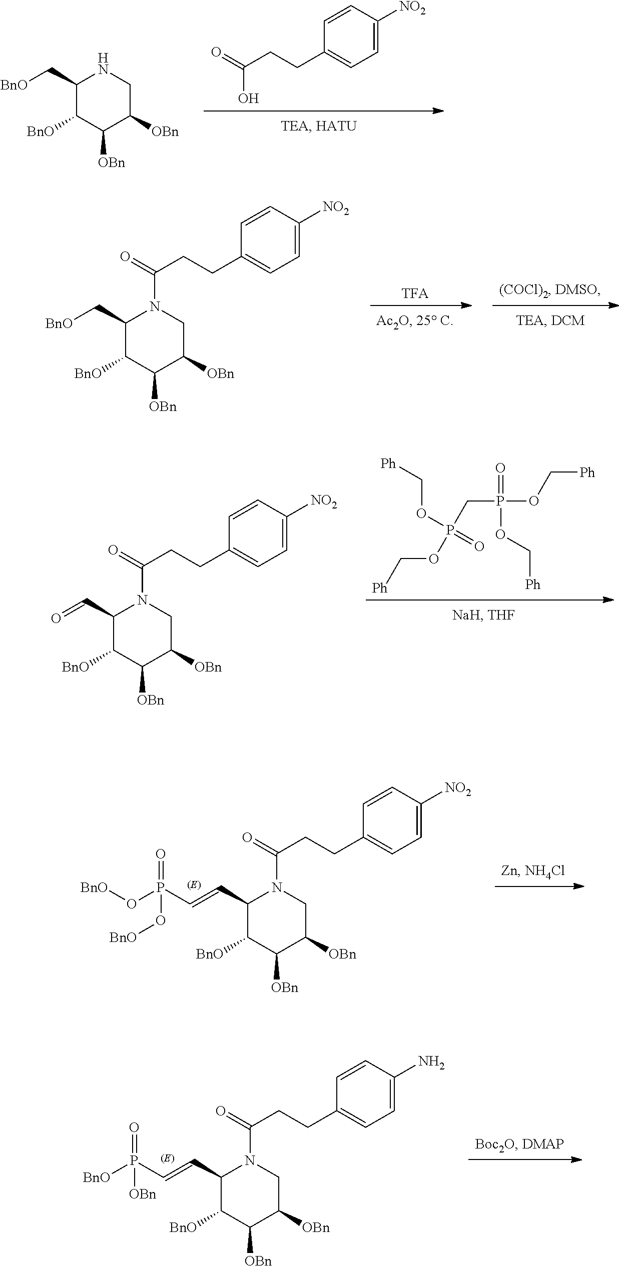

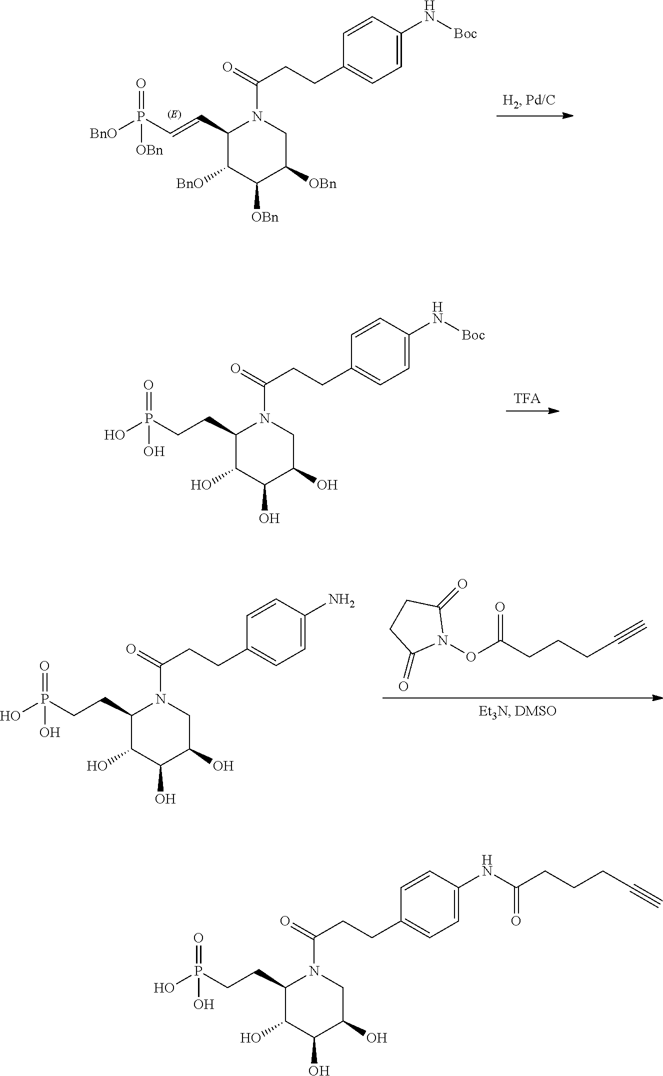

### Preparation of N-((2-((2R,3S,4S,5S)-3,4,5,6-tetrahydroxytetrahydro-2H-pyran-2-yl)ethyl)sulfonyl) pent-4-ynamide (A67)

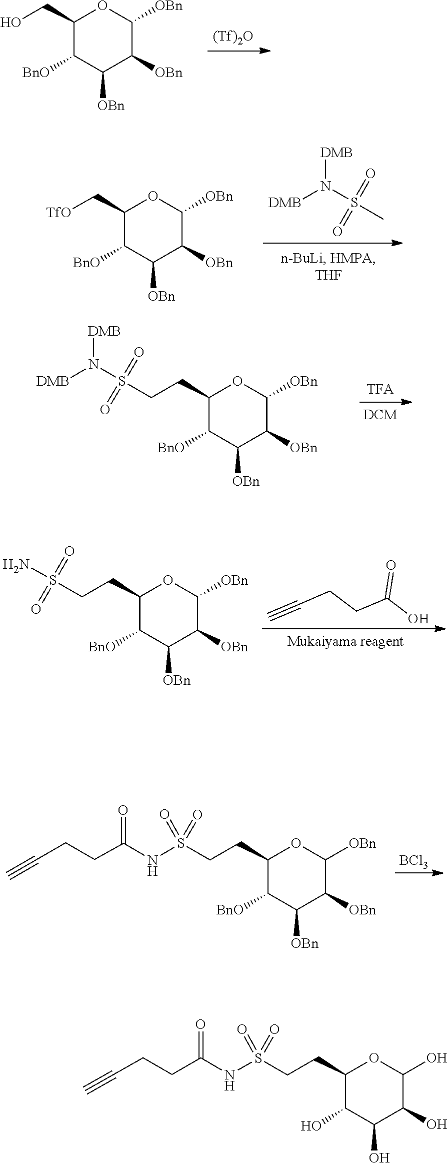

### Preparation of imino (pent-4-yn-1-yl) (2-((2R,3S,4S,5S,6S)-3,4,5,6-tetrahydroxytetrahydro-2H-pyran-2-yl)ethyl)-16-sulfanone (A68)

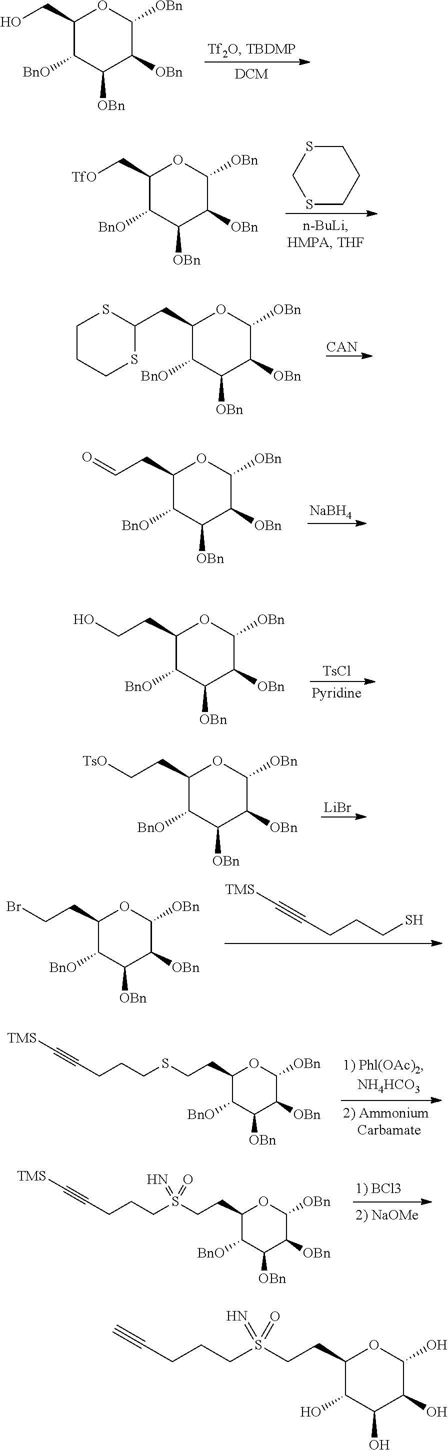

### Preparation of (2-((2R,3S,4S,5S,6R)-6-(1-(3-(hex-5-ynamido)phenyl)-1H-1,2,3-triazol-4-yl)-3,4,5-trihydroxytetrahydro-2H-pyran-2-yl)ethyl)phosphonic acid (A69)

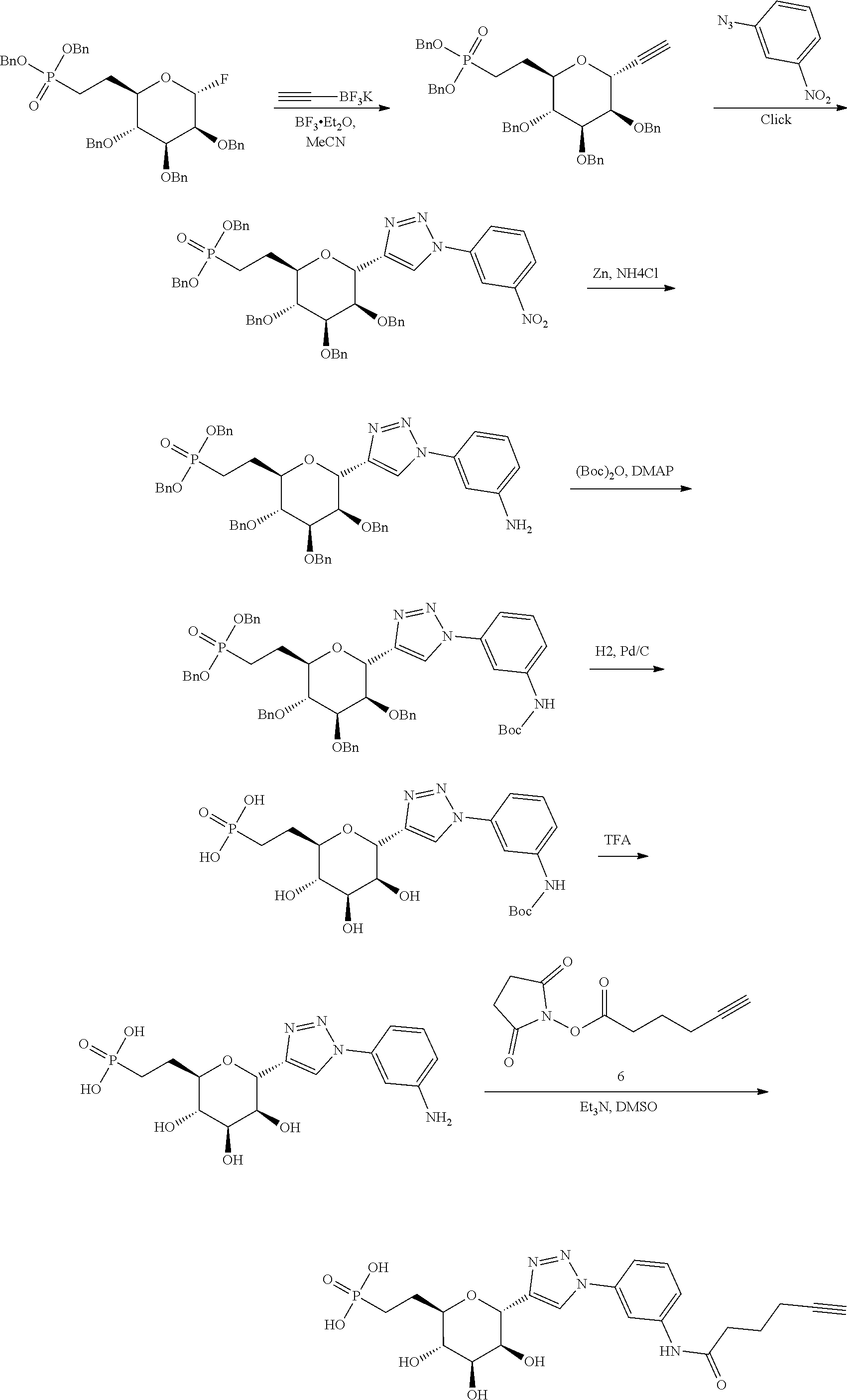

### Preparation of (2-((2R,3S,4S,5S,6R)-3,4,5-trihydroxy-6-(1-(naphthalen-2-yl)-1H-1,2,3-triazol-4-yl)tetrahydro-2H-pyran-2-yl)ethyl)phosphonic acid (A70)

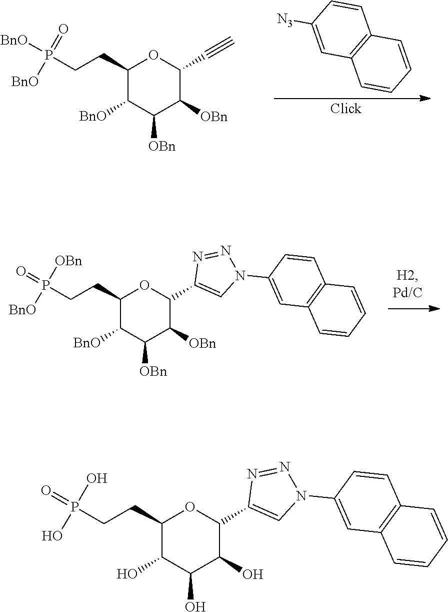

### Preparation of (2-((2R,3S,4S,5S,6R)-6-(1-cyclohexyl-1H-1,2,3-triazol-4-yl)-3,4,5-trihydroxytetrahydro-2H-pyran-2-yl)ethyl)phosphonic acid (A71)

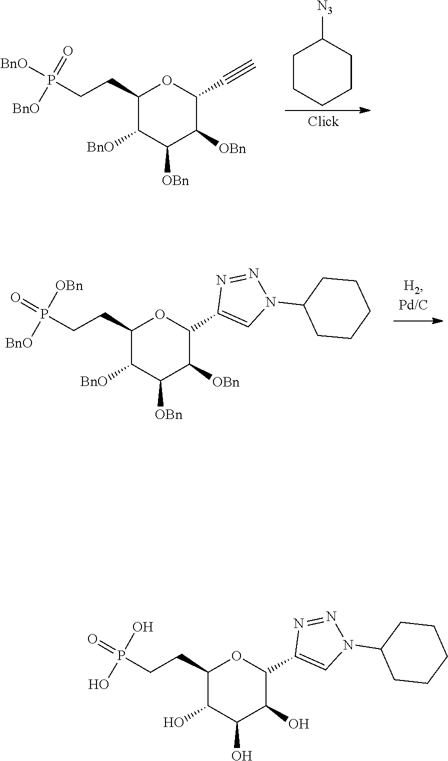

### Preparation of (2-((2R,3S,4S,5S)-6-((9H-fluoren-2-yl)oxy)-3,4,5-trihydroxytetrahydro-2H-pyran-2-yl)ethyl) (2,6-difluorobenzyl)phosphinic acid (A72)

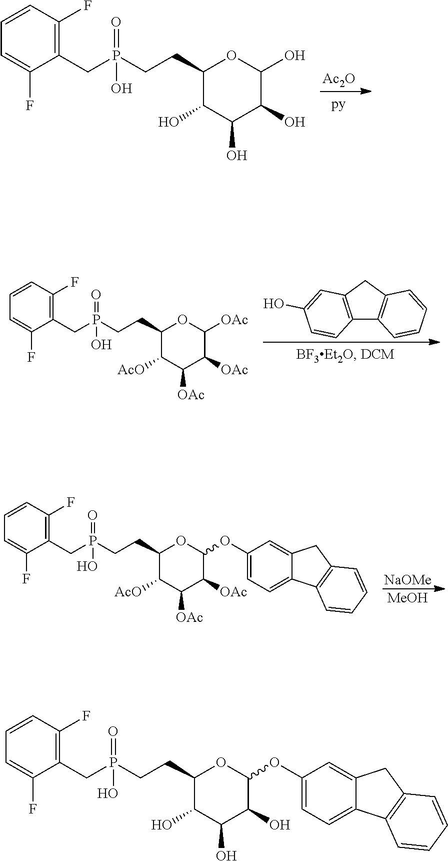

Step 1: To a solution of 1 (1.00 eq) in pyridine (1 mL) was added Ac₂O (10.00 eq) dropwise at 0° C. The reaction was stirred at 25° C. for 18 hours. LCMS showed the desired mass was detected. The resulting mixture was concentrated in vacuo. The crude product was purified by reverse phase (5-40% MeCN in water, 0.1% TFA) to give 2 as a colorless oil. Yield: 31.6%. LC-MS (ESI) found: 537 [M+H]⁺.

Step 2: To a solution of 2 (1.00 eq) and 3 (2.00 eq) in CH₃CN (1 mL) was added BF₃·Et₂O(0.50 eq) dropwise at 25° C. The reaction was stirred at 25° C. for 0.5 hour. LCMS showed the desired mass was detected. The mixture was quenched with Et₃N (10.0 eq), then concentrated in vacuo.

The crude product was purified by reverse phase (5-50% MeCN in water, 0.1% TFA) to give 4 as a colorless oil. Yield: 35.4%. LC-MS (ESI) found: 659 [M+H]⁺.

Step 3: To a solution of 4 (1.00 eq) in MeOH (1 mL) was added NaOMe (0.50 eq) at 0° C. The mixture was stirred at 25° C. for 0.5 hour. LCMS showed the desired mass was detected. The resulting mixture was neutralized by AMBERLITE IR-120, then filtered, the filtrate was concentrated in vacuo. The crude product was purified by prep-HPLC to give A108 as a white solid. Yield: 28.5%. LCMS found: [M+H]⁺=533. ¹H NMR (400 MHz, CD₃OD): δ 7.73 (t, J=7.8 Hz, 2H), 7.52 (d, J=7.4 Hz, 1H), 7.34 (t, J=7.2 Hz, 1H), 7.28-7.21 (m, 2H), 7.17-7.10 (m, 1H), 7.07 (dd, J=8.4, 2.3 Hz, 1H), 6.79 (t, J=7.8 Hz, 2H), 5.55 (d, J=1.6 Hz, 1H), 4.04 (dd, J=3.4, 1.8 Hz, 1H), 3.88 (dd, J=8.8, 3.2 Hz, 1H), 3.85 (s, 2H), 3.57-3.47 (m, 2H), 3.11 (d, J=16.7 Hz, 2H), 2.20-2.08 (m, 1H), 1.93-1.82 (m, 1H), 1.77-1.66 (m, 1H), 1.48-1.36 (m, 1H). ¹⁹F NMR (377 MHz, CD₃OD): 8-114.61 (d, J=3.6 Hz). ³¹P NMR (162 MHz, CD₃OD): δ 48.83 (s).

### Preparation of 1-(((2,6-difluorobenzyl) (2-((2R,3R,4S,5S,6R)-3,4,5-tris((3,4-dimethoxybenzyl)oxy)-6-(4-methoxyphenoxy)tetrahydro-2H-pyran-2-yl)ethyl)phosphoryl)oxy)-2-methylpropyl propionate (A73)

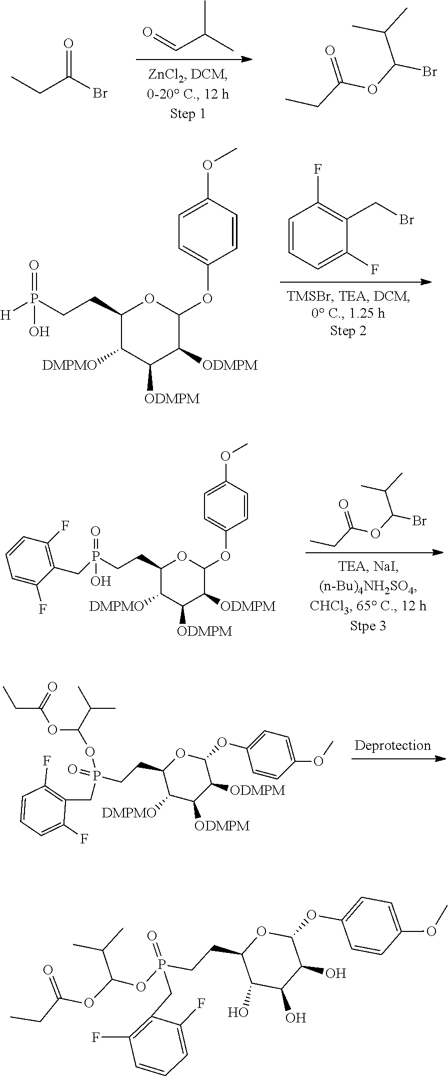

Step 1: To a solution of propanoyl bromide (2 g, 14.6 mmol) in DCM (20 mL) was added ZnCl₂ (995 mg, 7.30 mmol). Then 2-methylpropanal (1.05 g, 14.6 mmol) was added under 0° C. The mixture was stirred at 20° C. for 12 hours. The reaction mixture was quenched by addition addition saturated NaHCO₃ aqueous solution 40 mL at 20° C., and extracted with DCM (40 mL*3). The combined organic layers were washed with brine 40 mL, dried over anhydrous Na₂SO₄, filtered and concentrated under reduced pressure to afford (1-bromo-2-methyl-propyl)propanoate (4 g, crude) as yellow oil.

Step 2: A mixture of 2-[(2R,3R,4S,5S,6R)-3,4,5-tris[(3,4-dimethoxyphenyl)methoxy]-6-(4-methoxyphenoxy)tetrahydropyran-2-yl]ethylphosphinic acid (200 mg, 250 μmol), TEA (126 mg, 1.25 mmol) and TMSBr (191 mg, 1.25 mmol) in DCM (4 mL) was degassed and purged with N₂ for 3 times. Then the mixture was stirred at 0° C. for 15 min under N₂ atmosphere. Then 2-(bromomethyl)-1,3-difluoro-benzene (103 mg, 500 μmol) was added. The mixture was stirred at 0° C. for 1 hour under N₂ atmosphere. LC-MS (ESI) found: 925 [M+H]⁺. The mixture was concentrated under reduced pressure to give a residue. The residue was purified by prep-HPLC (Welch Xtimate C18 150*25 mm*5 μm; mobile phase: [water (TFA)−ACN]; gradient: 52%-72% B over 3 min) to afford (2,6-difluorophenyl)methyl-[2-[(2R,3R,4S,5S)-3,4,5-tris[(3,4-dimethoxyphenyl)methoxy]-6-(4-methoxyphenoxy)tetrahydropyran-2-yl]ethyl]phosphinic acid (140 mg, 60.4% yield) as a white solid. LC-MS (ESI) found: 925 [M+H]⁺.

Step 3: To a solution of (2,6-difluorophenyl)methyl-[2-[(2R,3R,4S,5S)-3,4,5-tris[(3,4-dimethoxyphenyl)methoxy]-6-(4-methoxyphenoxy)tetrahydropyran-2-yl]ethyl]phosphinic acid (20 mg, 21.6 μmol) in CHCl₃ (2 mL) was added (1-bromo-2-methyl-propyl)propanoate (22.6 mg, 108 μmol), NaI (1.62 mg, 10.8 μmol), hydrogen sulfate; tetrabutylammonium (3.67 mg, 10.8 μmol) and TEA (10.9 mg, 108 μmol). The mixture was stirred at 65° C. for 12 hours. LC-MS (ESI) found: 1054 [M+H]⁺. The reaction mixture was diluted with H₂O₁₀ mL at 0° C., and extracted with EtOAc 30 mL (10 mL*3). The combined organic layers were washed with brine 10 mL, dried over Na₂SO₄, filtered and concentrated under reduced pressure to give a residue. The residue was purified by prep-HPLC (Welch Xtimate C18 150*25 mm*5 μm; mobile phase: [water (TFA)-ACN]; gradient: 58%-88% B over 10 min) to afford [1-[(2,6-difluorophenyl)methyl-[2-[(2R,3R,4S,5S,6R)-3,4,5-tris[(3,4-dimethoxyphenyl)methoxy]-6-(4-methoxyphenoxy)tetrahydropyran-2-yl]ethyl]phosphoryl]oxy-2-methyl-propyl]propanoate (5 mg, 21.9% yield) as a white solid. LC-MS (ESI) found: 1054 [M+H]⁺.

### Preparation of (hydroxy (phenyl)methyl) (2-((2R,3S,4S,5S)-3,4,5-trihydroxy-6-(4-methoxyphenoxy)tetrahydro-2H-pyran-2-yl)ethyl)phosphinic acid (A74)

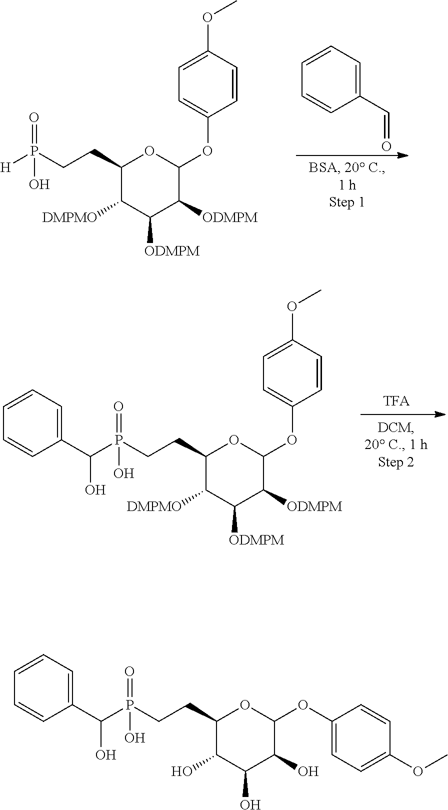

Step 1: A mixture of 2-[(2R,3R,4S,5S,6R)-3,4,5-tris[(3,4-dimethoxyphenyl)methoxy]-6-(4-methoxyphenoxy)tetrahydropyran-2-yl]ethylphosphinic acid (100 mg, 125.1 μmol), benzaldehyde (26.5 mg, 250 μmol) and BSA (178 mg, 876 μmol) in DCM (2 mL) was degassed and purged with N₂ for 3 times, and then the mixture was stirred at 0° C. for 1 hour under N₂ atmosphere. LC-MS (ESI) found: 905 [M+H]⁺. The mixture was concentrated under reduced pressure to give a residue.

The residue was purified by prep-HPLC (Welch Xtimate C18 150*25 mm*5 μm; mobile phase: [water (TFA)−ACN]; gradient: 43%-73% B over 3 min) to afford [hydroxy (phenyl)methyl]-[2-[(2R,3R,4S,5S)-3,4,5-tris[(3,4-dimethoxyphenyl)methoxy]-6-(4-methoxyphenoxy)tetrahydropyran-2-yl]ethyl]phosphinic acid (70 mg, 61.7% yield) as a white solid. LC-MS (ESI) found: 905 [M+H]⁺.

Step 2: To a solution of [hydroxy (phenyl)methyl]-[2-[(2R,3R,4S,5S)-3,4,5-tris[(3,4-dimethoxyphenyl)methoxy]-6-(4-methoxyphenoxy)tetrahydropyran-2-yl]ethyl]phosphinic acid (60 mg, 66.3 μmol) in DCM (1 mL) was added TFA (1 mL). The mixture was stirred at 20° C. for 1 hour. LC-MS (ESI) found: 455 [M+H]⁺. The reaction mixture was concentrated under reduced pressure to give a residue. The residue was purified by prep-HPLC (Welch Ultimate C18 150*25 mm*5 μm; mobile phase: [water (TFA)−ACN];gradient: 10%-40% B over 10 min) to afford [hydroxy (phenyl)methyl]-[2-[(2R,3S,4S,5S)-3,4,5-trihydroxy-6-(4-methoxyphenoxy)tetrahydropyran-2-yl]ethyl]phosphinic acid (12 mg, 39.8% yield) as a white solid. ¹H NMR (400 MHz, MeOD): δ 7.45-7.23 (m, 5H), 7.00-6.92 (m, 2H), 6.87-6.80 (m, 2H), 5.34 (dd, J=1.2, 11.2 Hz, 1H), 4.85-4.79 (m, 1H), 3.98 (td, J=1.6, 3.2 Hz, 1H), 3.82 (td, J=2.8, 9.2 Hz, 1H), 3.76 (s, 3H), 3.57-3.39 (m, 2H), 1.84-1.64 (m, 1H), 2.19-1.24 (m, 2H), 1.58-1.22 (m, 1H) and LC-MS (ESI) found: 455 [M+H]⁺.

### Preparation of (cyclopent-1-en-1-ylmethyl) (2-((2R,3S,4S,5S)-3,4,5-trihydroxy-6-(4-methoxyphenoxy)tetrahydro-2H-pyran-2-yl)ethyl)phosphinic acid (A75)

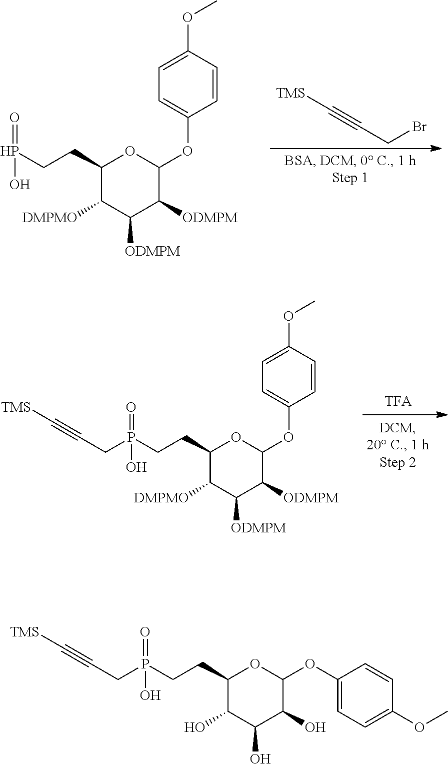

Step 1: A mixture of 2-[(2R,3R,4S,5S,6R)-3,4,5-tris[(3,4-dimethoxyphenyl)methoxy]-6-(4-methoxyphenoxy)tetrahydropyran-2-yl]ethylphosphinic acid (100 mg, 125 μmol), 3-bromoprop-1-ynyl(trimethyl) silane (47.8 mg, 250 μmol) and BSA (178 mg, 876 μmol) in DCM (2 mL) was degassed and purged with N₂ for 3 times, and then the mixture was stirred at 0° C. for 1 hour under N₂ atmosphere. LC-MS (ESI) found: 910 [M+H]⁺. The mixture was concentrated under reduced pressure to give a residue. The residue was purified by prep-HPLC (Welch Xtimate C18 150*25 mm*5 μm; mobile phase: [water (TFA)−ACN]; gradient: 56%-76% B over 3 min) to afford 3-trimethylsilylprop-2-ynyl-[2-[(2R,3R,4S,5S)-3,4,5-tris[(3,4-dimethoxyphenyl)methoxy]-6-(4-methoxyphenoxy)tetrahydropyran-2-yl]ethyl]phosphinic acid (60 mg, 52.7% yield) as a white solid. ¹H NMR (400 MHz, CDCl₃): δ 6.94-6.79 (m, 13H), 5.35 (d, J=1.6 Hz, 1H), 4.92-4.61 (m, 6H), 4.06 (dd, J=2.8, 8.8 Hz, 1H), 3.91-3.89 (m, 4H), 3.87 (s, 3H), 3.85 (s, 3H), 3.80 (d, J=2.4 Hz, 6H), 3.78 (s, 3H), 3.76-3.71 (m, 4H), 3.69-3.62 (m, 1H), 2.69 (d, J=18.4 Hz, 2H), 2.21-2.10 (m, 1H), 2.04-2.00 (m, 1H), 1.89-1.76 (m, 1H), 1.73-1.61 (m, 1H), 0.00 (s, 9H) and LC-MS (ESI) found: 910 [M+H]⁺.

Step 2: To a solution of 3-trimethylsilylprop-2-ynyl-[2-[(2R,3R,4S,5S)-3,4,5-tris[(3,4-dimethoxyphenyl)methoxy]-6-(4-methoxyphenoxy)tetrahydropyran-2-yl]ethyl]phosphinic acid (50 mg, 55.0 μmol) in DCM (0.5 mL) was added TFA (0.5 mL). The mixture was stirred at 20° C. for 1 hour. LC-MS (ESI) found: 459 [M+H]⁺. The reaction mixture concentrated under reduced pressure to give a residue. The residue was purified by prep-HPLC (Welch Xtimate C18 150*25 mm*5 μm; mobile phase: [water (TFA)−ACN]; gradient: 27%-47% B over 3 min) to afford 2-[(2R,3S,4S,5S)-3,4,5-trihydroxy-6-(4-methoxyphenoxy)tetrahydropyran-2-yl]ethyl-(3-trimethylsilylprop-2-ynyl)phosphinic acid (6 mg, 23.3% yield) as a white solid. ¹H NMR (400 MHz, MeOD): δ 7.06-6.96 (m, 2H), 6.90-6.81 (m, 2H), 5.37 (d, J=1.6 Hz, 1H), 4.00 (dd, J=1.6, 3.2 Hz, 1H), 3.89-3.82 (m, 1H), 3.75 (s, 3H), 3.57-3.49 (m, 2H), 2.75 (d, J=18 Hz, 2H), 2.22-1.99 (m, 2H), 1.80-1.57 (m, 2H), 0.00 (s, 9H) and LC-MS (ESI) found: 459 [M+H]⁺.

### Preparation of (2-((2R,3S,4S,5S,6R)-6-((7-amino-9H-fluoren-2-yl)oxy)-3,4,5-trihydroxytetrahydro-2H-pyran-2-yl)ethyl)phosphonic acid (A76)

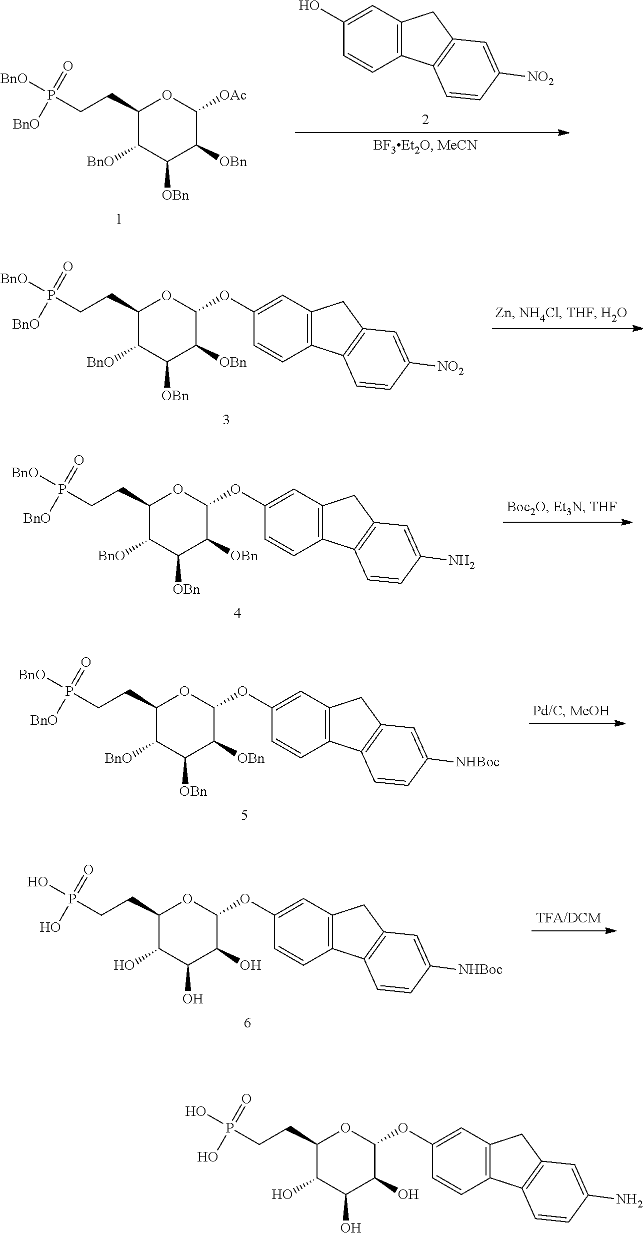

Step 1: To a solution of 1 (1.00 eq) and 2 (1.20 eq) in MeCN (2 mL) was added BF₃·Et₂O(0.1 mL) at 0° C. The mixture was stirred at 25° C. for 3 hours, then quenched with TEA. The mixture was concentrated and the residue was purified by flash (PE/EA=1:1) to afford 3 as yellow oil. Yield: 41%. LCMS found: [M+Na]⁺=940.

Step 2: A solution of 3 (1.00 eq), Zn (10.0 eq) and NH₄Cl (10.0 eq) in THF (5 mL) and H₂O (2 mL) was stirred at 60° C. for 10 hours. The mixture was filtered through a Celite pad, and the filtrate was concentrated to afford 4 as yellow oil. Yield: 83%. LCMS found: [M+H]⁺=888. Step 3: A solution of 4 (1.00 eq), Et₃N (3.00 eq) and (Boc)₂₀ (5.00 eq) in THF (2 mL) was stirred at 20° C. for 10 hours. The mixture was concentrated and the residue was purified by flash (PE/EA=2:1) to afford 5 as yellow oil. Yield: 91%. LCMS found: [M+H]⁺=988.

Step 4: To a solution of 5 (1.00 eq) in MeOH (5 mL) was added Pd/C (0.1 eq) at 25° C. The mixture was stirred at 25° C. under a H₂ balloon for 16 hours. The mixture was filtered through a Celite pad, and the filtrate was concentrated to afford 6 as yellow oil. Yield: 71%. LCMS found: [M+H]⁺=538.

Step 5: To a solution of 6 (1.00 eq) in DCM (3 mL) was added TFA (1 mL) at 25° C. The mixture was stirred at 25° C. for 16 hours, then concentrated to afford A76 as yellow oil. Yield: 83%. LCMS found: [M+H]⁺=438. ¹H NMR (400 MHz, CD₃OD): δ 7.84 (d, J=8.0 Hz, 1H), 7.77 (d, J=8.0 Hz, 1H), 7.53 (s, 1H), 7.36 (dd, J=8.0, 1.6 Hz, 1H), 7.33 (d, J=1.6 Hz, 1H), 7.12 (dd, J=8.4, 2.0 Hz, 1H), 5.56 (d, J=1.2 Hz, 1H), 4.05 (dd, J=3.6, 1.6 Hz, 1H), 3.98-3.93 (m, 2H), 3.89 (dd, J=8.8, 3.2 Hz, 1H), 3.57 (t, J=9.6 Hz, 1H), 3.54-3.48 (m, 1H), 2.18-2.12 (m, 1H), 1.80-1.71 (m, 1H), 1.43-1.30 (m, 2H).

### Preparation of prop-2-yn-1-yl(2-((2R,3S,4S,5S)-3,4,5-trihydroxy-6-(4-methoxyphenoxy)tetrahydro-2H-pyran-2-yl)ethyl)phosphinic acid (A77)

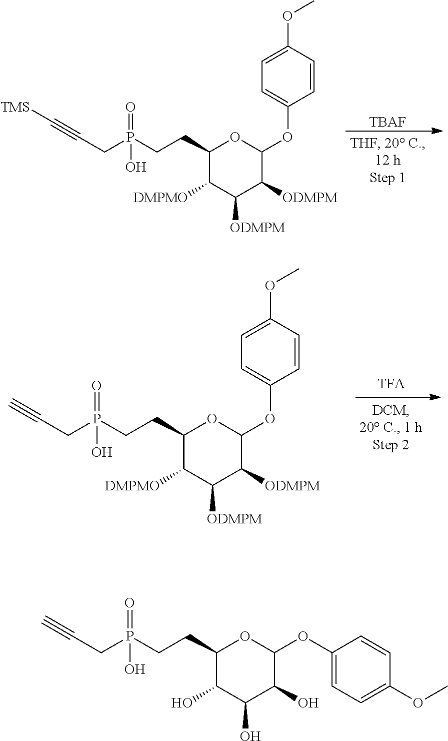

Step 1: To a solution of 3-trimethylsilylprop-2-ynyl-[2-[(2R,3R,4S,5S)-3,4,5-tris[(3,4-dimethoxyphenyl)methoxy]-6-(4-methoxyphenoxy)tetrahydropyran-2-yl]ethyl]phosphinic acid (100 mg, 110 μmol) in THF (2 mL) was added TBAF (1 M, 330.02 μL). The mixture was stirred at 20° C. for 12 hours. LC-MS (ESI) found: 837 [M+H]⁺. The reaction mixture was concentrated under reduced pressure to afford prop-2-ynyl-[2-[(2R,3R,4S,5S)-3,4,5-tris[(3,4-dimethoxyphenyl)methoxy]-6-(4-methoxyphenoxy)tetrahydropyran-2-yl]ethyl]phosphinic acid (120 mg, crude) as yellow oil. LC-MS (ESI) found: 837 [M+H]⁺.

Step 2: To a solution of prop-2-ynyl-[2-[(2R,3R,4S,5S)-3,4,5-tris[(3,4-dimethoxyphenyl)methoxy]-6-(4-methoxyphenoxy)tetrahydropyran-2-yl]ethyl]phosphinic acid (120 mg, 143 μmol) in DCM (1 mL) was added TFA (1 mL). The mixture was stirred at 20° C. for 1 hour. LC-MS (ESI) found: 387 [M+H]⁺. The reaction mixture was concentrated under reduced pressure to give a residue. The residue was purified by prep-HPLC (Welch Xtimate C18 150*25 mm*5 μm; mobile phase: [water (TFA)−ACN]; gradient: 7%-37% B over 3 min) to afford prop-2-ynyl-[2-[(2R,3S,4S,5S)-3,4,5-trihydroxy-6-(4-methoxyphenoxy)tetrahydropyran-2-yl]ethyl]phosphinic acid (11 mg, 18.0% yield) as a white solid. ¹H NMR (400 MHz, MeOD): δ 7.04-6.97 (m, 2H), 6.89-6.80 (m, 2H), 5.37 (d, J=1.6 Hz, 1H), 3.99 (dd, J=1.6, 3.2 Hz, 1H), 3.84 (dd, J=3.6, 8.8 Hz, 1H), 3.75 (s, 3H), 3.58-3.48 (m, 2H), 2.63 (dd, J=2.4, 18 Hz, 2H), 2.41 (td, J=2.8, 5.6 Hz, 1H), 2.21-1.93 (m, 2H), 1.79-1.52 (m, 2H) and LC-MS (ESI) found: 387 [M+H]⁺.

### Preparation of (cyclopent-1-en-1-ylmethyl) (2-((2R,3S,4S,5S)-3,4,5-trihydroxy-6-(4-methoxyphenoxy)tetrahydro-2H-pyran-2-yl)ethyl)phosphinic acid (A78)

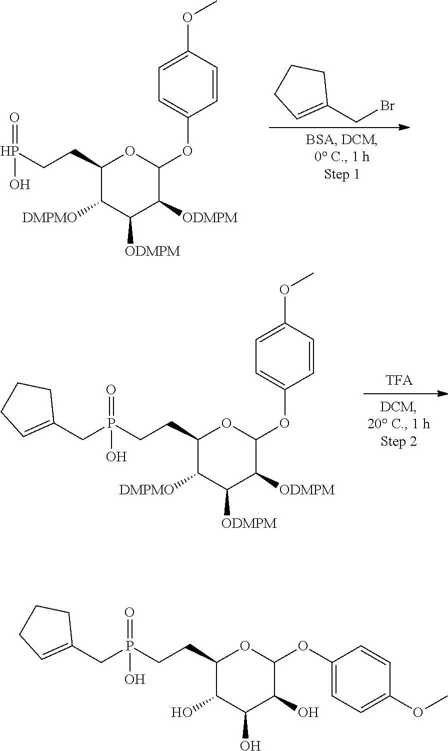

Step 1: A mixture of 2-[(2R,3R,4S,5S,6R)-3,4,5-tris[(3,4-dimethoxyphenyl)methoxy]-6-(4-methoxyphenoxy)tetrahydropyran-2-yl]ethylphosphinic acid (100 mg, 125 μmol), 1-(bromomethyl)cyclopentene (60.4 mg, 375 μmol) and BSA (178 mg, 876 μmol) in DCM (2 mL) was degassed and purged with N₂ for 3 times, and then the mixture was stirred at 0° C. for 1 hour under N₂ atmosphere. LC-MS (ESI) found: 879 [M+H]⁺. The mixture was concentrated under reduced pressure to give a residue. The residue was purified by prep-HPLC (Welch Xtimate C18 150*25 mm*5 μm; mobile phase: [water (TFA)−ACN]; gradient: 53%-73% B over 3 min) to afford cyclopenten-1-ylmethyl-[2-[(2R,3R,4S,5S)-3,4,5-tris[(3,4-dimethoxyphenyl)methoxy]-6-(4-methoxyphenoxy)tetrahydropyran-2-yl]ethyl]phosphinic acid (50 mg, 45.4% yield) as a white solid. ¹H NMR (400 MHz, CDCl₃): δ 6.95-6.74 (m, 14H), 5.36 (s, 1H), 4.91-4.60 (m, 6H), 4.05 (dd, J=2.7, 9.2 Hz, 1H), 3.92-3.87 (m, 7H), 3.84 (s, 3H), 3.82-3.77 (m, 9H), 3.76-3.69 (m, 4H), 3.63-3.57 (m, 1H), 2.55 (d, J=17.6 Hz, 2H), 2.33-2.20 (m, 4H), 2.18-2.07 (m, 1H), 1.93-1.71 (m, 4H), 1.50-1.40 (m, 1H) and LC-MS (ESI) found: 879 [M+H]⁺.

Step 2: To a solution of cyclopenten-1-ylmethyl-[2-[(2R,3R,4S,5S)-3,4,5-tris[(3,4-dimethoxyphenyl)methoxy]-6-(4-methoxyphenoxy)tetrahydropyran-2-yl]ethyl]phosphinic acid (40 mg, 45.5 μmol) in DCM (0.5 mL) was added TFA (0.5 mL). The mixture was stirred at 20° C. for 1 hour. LC-MS (ESI) found: 429 [M+H]⁺. The reaction mixture was concentrated under reduced pressure to give a residue. The residue was purified by prep-HPLC (Welch Xtimate C18 150*25 mm*5 μm; mobile phase: [water (TFA)−ACN]; gradient: 20%-40% B over 10 min) to afford cyclopenten-1-ylmethyl-[2-[(2R,3S,4S,5S)-3,4,5-trihydroxy-6-(4-methoxyphenoxy)tetrahydropyran-2-yl]ethyl]phosphinic acid (12 mg, 61.5% yield) as a white solid. ¹H NMR (400 MHz, CDCl₃): δ 7.04-6.94 (m, 2H), 6.88-6.79 (m, 2H), 5.53 (s, 1H), 5.38 (d, J=1.2 Hz, 1H), 3.99 (dd, J=1.6, 3.2 Hz, 1H), 3.83 (dd, J=3.6, 8.8 Hz, 1H), 3.75 (s, 3H), 3.56-3.45 (m, 2H), 2.60 (d, J=18.0 Hz, 2H), 2.42-2.29 (m, 4H), 2.19-2.03 (m, 1H), 1.90-1.79 (m, 3H), 1.72-1.61 (m, 1H), 1.51-1.33 (m, 1H) and LC-MS (ESI) found: 429 [M+H]⁺.

### Preparation of (2-((2R,3S,4S,5S,6R)-6-((9H-fluoren-2-yl)oxy)-3,4,5-trihydroxytetrahydro-2H-pyran-2-yl)ethyl) (2,6-difluorobenzyl)phosphinic acid (A79)

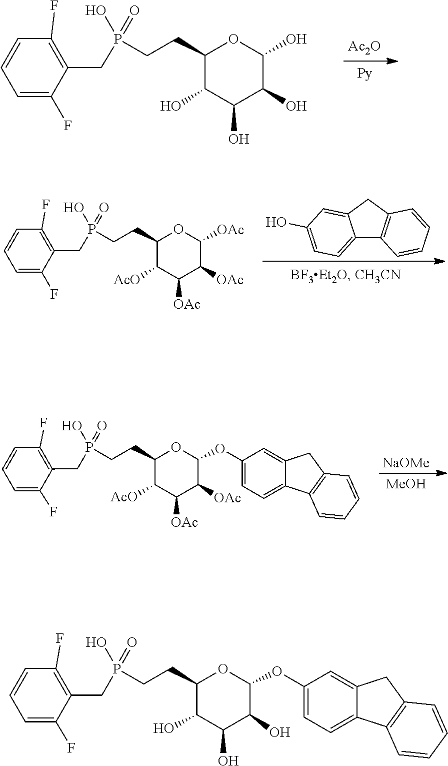

Step 1: To a solution of (2,6-difluorobenzyl) (2-((2R,3S,4S,5S,6S)-3,4,5,6-tetrahydroxytetrahydro-2H-pyran-2-yl)ethyl)phosphinic acid (1.00 eq) in pyridine (1 mL) was added Ac₂₀ (10.00 eq) dropwise at 0° C. The reaction was stirred at 25° C. for 18 hours. LCMS showed the desired mass was detected. The resulting mixture was concentrated in vacuo. The crude product was purified by reverse phase (5-40% MeCN in water, 0.1% TFA) to give 2 as a colorless oil. Yield: 31.6%. LC-MS (ESI) found: 537 [M+H]⁺.

Step 2: To a solution of (2,6-difluorobenzyl) (2-((2R,3R,4S,5S,6R)-3,4,5,6-tetraacetoxytetrahydro-2H-pyran-2-yl)ethyl)phosphinic acid (1.00 eq) and 9H-fluoren-2-ol (2.00 eq) in CH₃CN (1 mL) was added BF₃·Et₂O(0.50 eq) dropwise at 25° C. The reaction was stirred at 25° C. for 0.5 hour. LCMS showed the desired mass was detected. The mixture was quenched with Et₃N (10.0 eq), then concentrated in vacuo. The crude product was purified by reverse phase (5-50% MeCN in water, 0.1% TFA) to give (2-((2R,3R,4S,5S,6R)-6-((9H-fluoren-2-yl)oxy)-3,4,5-triacetoxytetrahydro-2H-pyran-2-yl)ethyl) (2,6-difluorobenzyl)phosphinic acid as a colorless oil. Yield: 35.4%. LC-MS (ESI) found: 659 [M+H]⁺.

Step 3: To a solution of (2-((2R,3R,4S,5S,6R)-6-((9H-fluoren-2-yl)oxy)-3,4,5-triacetoxytetrahydro-2H-pyran-2-yl)ethyl) (2,6-difluorobenzyl)phosphinic acid (1.00 eq) in MeOH (1 mL) was added NaOMe (0.50 eq) at 0° C. The mixture was stirred at 25° C. for 0.5 hour. LCMS showed the desired mass was detected. The resulting mixture was neutralized by AMBERLITE IR-120, then filtered, the filtrate was concentrated in vacuo. The crude product was purified by prep-HPLC to give A79 as a white solid. Yield: 28.5%. LCMS found: [M+H]⁺=533. ¹H NMR (400 MHz, CD₃OD): δ 7.73 (t, J=7.8 Hz, 2H), 7.52 (d, J=7.4 Hz, 1H), 7.34 (t, J=7.2 Hz, 1H), 7.28-7.21 (m, 2H), 7.17-7.10 (m, 1H), 7.07 (dd, J=8.4, 2.3 Hz, 1H), 6.79 (t, J=7.8 Hz, 2H), 5.55 (d, J=1.6 Hz, 1H), 4.04 (dd, J=3.4, 1.8 Hz, 1H), 3.88 (dd, J=8.8, 3.2 Hz, 1H), 3.85 (s, 2H), 3.57-3.47 (m, 2H), 3.11 (d, J=16.7 Hz, 2H), 2.20-2.08 (m, 1H), 1.93-1.82 (m, 1H), 1.77-1.66 (m, 1H), 1.48-1.36 (m, 1H). ¹⁹F NMR (377 MHz, CD₃OD): 8-114.61 (d, J=3.6 Hz). 31p NMR (162 MHz, CD₃OD): δ 48.83 (s).

### Preparation of (2,2,2-trifluoroethyl) (2-((2R,3S,4S,5S)-3,4,5-trihydroxy-6-(4-methoxyphenoxy)tetrahydro-2H-pyran-2-yl)ethyl)phosphinic acid (A80)

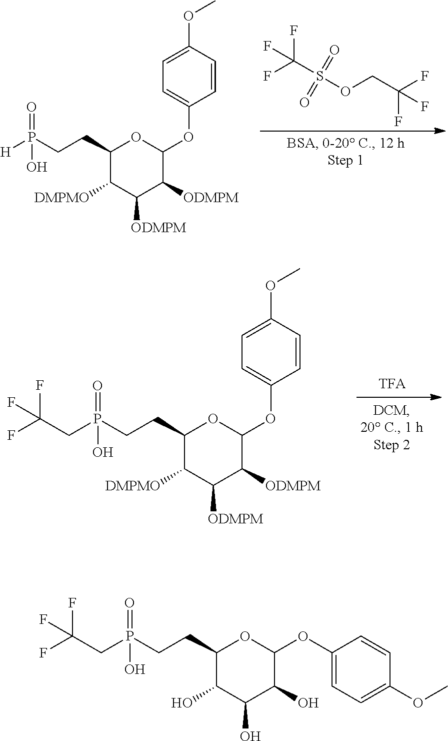

Step 1: A mixture of 2-[(2R,3R,4S,5S,6R)-3,4,5-tris[(3,4-dimethoxyphenyl)methoxy]-6-(4-methoxyphenoxy)tetrahydropyran-2-yl]ethylphosphinic acid (100 mg, 125 μmol), 2,2,2-trifluoroethyl trifluoromethanesulfonate (58.1 mg, 250 μmol) and BSA (178 mg, 876 μmol) in DCM (2 mL) was degassed and purged with N₂ for 3 times, and then the mixture was stirred at 0° C. for 1 hour under N₂ atmosphere. Then the mixture was stirred at 20° C. for 11 hours under N₂ atmosphere. LC-MS (ESI) found: 881 [M+H]⁺. The reaction mixture was concentrated under reduced pressure to give a residue. The residue was purified by prep-HPLC (Welch Xtimate C18 150*25 mm*5 μm; mobile phase: [water (TFA)−ACN];gradient: 42%-72% B over 10 min) to afford 2,2,2-trifluoroethyl-[2-[(2R,3R,4S,5S)-3,4,5-tris[(3,4-dimethoxyphenyl)methoxy]-6-(4-methoxyphenoxy)tetrahydropyran-2-yl]ethyl]phosphinic acid (12 mg, 10.8% yield) as a yellow solid. ¹H NMR (400 MHz, CDCl₃): δ 6.97-6.77 (m, 13H), 5.34 (s, 1H), 4.92-4.59 (m, 6H), 4.06 (dd, J=2.8, 8.8 Hz, 1H), 3.93-3.89 (m, 4H), 3.87 (s, 3H), 3.85 (s, 3H), 3.83-3.78 (m, 9H), 3.76-3.69 (m, 4H), 3.67-3.63 (m, 1H), 2.71-2.49 (m, 2H), 2.13-1.99 (m, 1H), 1.90-1.47 (m, 3H) and LC-MS (ESI) found: 881 [M+H]⁺.

Step 2: To a solution of 2,2,2-trifluoroethyl-[2-[(2R,3R,4S,5S)-3,4,5-tris[(3,4-dimethoxyphenyl)methoxy]-6-(4-methoxyphenoxy)tetrahydropyran-2-yl]ethyl]phosphinic acid (12 mg, 13.6 μmol) in DCM (0.5 mL) was added TFA (0.5 mL). The mixture was stirred at 20° C. for 1 hour. LC-MS (ESI) found: 431 [M+H]⁺. The reaction mixture was concentrated under reduced pressure to give a residue. The residue was purified by prep-HPLC (Welch Ultimate C18 150*25 mm*5 μm; mobile phase: [water (TFA)−ACN]; gradient: 10%-40% B over 10 min) to afford 2,2,2-trifluoroethyl-[2-[(2R,3S,4S,5S)-3,4,5-trihydroxy-6-(4-methoxyphenoxy)tetrahydropyran-2-yl]ethyl]phosphinic acid (1 mg, 15.8% yield) as a white solid.

¹H NMR (400 MHz, MeOD): δ 6.99 (d, J=9.2 Hz, 2H), 6.89-6.80 (m, 2H), 5.37 (d, J=1.6 Hz, 1H), 3.99 (dd, J=1.6, 3.2 Hz, 1H), 3.84 (dd, J=3.2, 8.8 Hz, 1H), 3.75 (s, 3H), 3.58-3.47 (m, 2H), 2.83-2.68 (m, 2H), 2.19-2.06 (m, 1H), 2.03-1.89 (m, 1H), 1.80-1.64 (m, 1H), 1.61-1.45 (m, 1H) and LC-MS (ESI) found: 431 [M+H]⁺.

### Preparation of benzyl(2-((2R,3S,4S,5S)-3,4,5-trihydroxy-6-(4-methoxyphenoxy)tetrahydro-2H-pyran-2-yl)ethyl)phosphinic acid (A81)

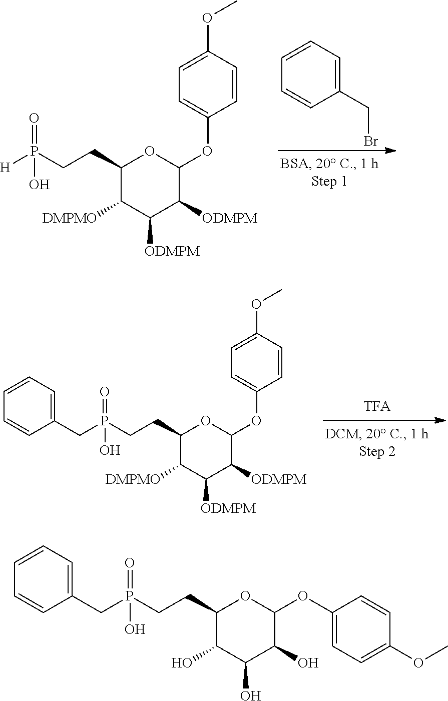

Step 1: A mixture of 2-[(2R,3R,4S,5S,6R)-3,4,5-tris[(3,4-dimethoxyphenyl)methoxy]-6-(4-methoxyphenoxy)tetrahydropyran-2-yl]ethylphosphinic acid (100 mg, 125 μmol), bromomethylbenzene (42.8 mg, 250 μmol) and BSA (178 mg, 876 μmol) in DCM (2 mL) was degassed and purged with N₂ for 3 times, and then the mixture was stirred at 0° C. for 1 hour under N₂ atmosphere. LC-MS (ESI) found: 889 [M+H]⁺. The reaction mixture was concentrated under reduced pressure to give a residue. The residue was purified by prep-HPLC (Welch Xtimate C18 150*25 mm*5 μm; mobile phase: [water (TFA)−ACN]; gradient: 50%-70% B over 3 min) to afford benzyl-[2-[(2R,3R,4S,5S)-3,4,5-tris[(3,4-dimethoxyphenyl)methoxy]-6-(4-methoxyphenoxy)tetrahydropyran-2-yl]ethyl]phosphinic acid (80 mg, 71.8% yield) as a white solid. ¹H NMR (400 MHz, CDCl₃): δ 7.24-7.11 (m, 5H), 6.94-6.78 (m, 13H), 5.33 (d, J=1.2 Hz, 1H), 4.91-4.61 (m, 6H), 4.08-4.01 (m, 1H), 3.91-3.87 (m, 7H), 3.84 (s, 3H), 3.81-3.76 (m, 12H), 3.69 (t, J=9.4 Hz, 1H), 3.61-3.53 (m, 1H), 2.93 (d, J=16.4 Hz, 2H), 2.14-1.99 (m, 1H), 1.83-1.63 (m, 2H), 1.45-1.31 (m, 1H) and LC-MS (ESI) found: 889 [M+H]⁺.

Step 2: To a solution of benzyl-[2-[(2R,3R,4S,5S)-3,4,5-tris[(3,4-dimethoxyphenyl)methoxy]-6-(4-methoxyphenoxy)tetrahydropyran-2-yl]ethyl]phosphinic acid (60 mg, 67.5 μmol) in DCM (1 mL) was added TFA (1 mL). The mixture was stirred at 20° C. for 1 hour. LC-MS (ESI) found: 439 [M+H]⁺. The reaction mixture was concentrated under reduced pressure to give a residue. The residue was purified by prep-HPLC (Welch Ultimate C18 150*25 mm*5 μm; mobile phase: [water (TFA)−ACN]; gradient: 17%-47% B over 10 min) to afford benzyl-[2-[(2R,3S,4S,5S)-3,4,5-trihydroxy-6-(4-methoxyphenoxy)tetrahydropyran-2-yl]ethyl]phosphinic acid (10 mg, 33.4% yield) as a white solid. ¹H NMR (400 MHz, MeOD): δ 7.36-7.19 (m, 5H), 7.00-6.89 (m, 2H), 6.86-6.77 (m, 2H), 5.35 (d, J=1.2 Hz, 1H), 3.98 (dd, J=1.6, 3.2 Hz, 1H), 3.81 (dd, J=3.2, 9.0 Hz, 1H), 3.76 (s, 3H), 3.56-3.41 (m, 2H), 3.09 (dd, J=2.8, 17.2 Hz, 2H), 2.16-2.03 (m, 1H), 1.86-1.60 (m, 2H), 1.42-1.27 (m, 1H) and LC-MS (ESI) found: 439 [M+H]⁺.

### Preparation of (cyclopentylmethyl) (2-((2R,3S,4S,5S)-3,4,5-trihydroxy-6-(4-methoxyphenoxy)tetrahydro-2H-pyran-2-yl)ethyl)phosphinic acid (A82)

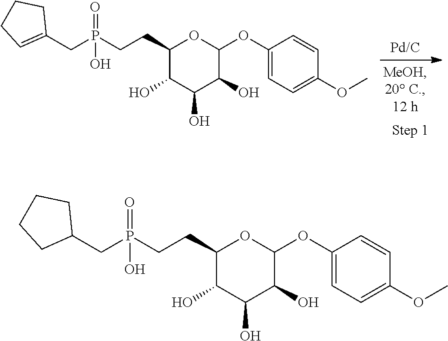 [SMILES: COc1ccc(OC2O[C@H](CCP(=O)(O)CC3=CCCC3)[C@@H](O)[C@H](O)[C@@H]2O)cc1 ; COc1ccc(OC2O[C@H](CCP(=O)(O)CC3CCCC3)[C@@H](O)[C@H](O)[C@@H]2O)cc1]

Step 1: A mixture of cyclopenten-1-ylmethyl-[2-[(2R,3S,4S,5S)-3,4,5-trihydroxy-6-(4-methoxyphenoxy)tetrahydropyran-2-yl]ethyl]phosphinic acid (5 mg, 11.6 μmol) and Pd/C (12.4 mg, 10% purity) in MeOH (1 mL) was degassed and purged with H₂ (15 Psi) for 3 times, and then the mixture was stirred at 20° C. for 12 hours under H₂ (15 Psi) atmosphere. LC-MS (ESI) found: 431 [M+H]⁺. The mixture filtered and concentrated under reduced pressure to afford cyclopentylmethyl-[2-[(2R,3S,4S,5S)-3,4,5-trihydroxy-6-(4-methoxyphenoxy)tetrahydropyran-2-yl]ethyl]phosphinic acid (4 mg, 79.6% yield) as a white solid. ¹H NMR (400 MHz, MeOD): δ 7.00 (d, J=8.8 Hz, 2H), 6.84 (d, J=8.8 Hz, 2H), 5.37 (s, 1H), 3.99 (s, 1H), 3.83 (d, J=5.6 Hz, 1H), 3.74 (s, 3H), 3.62-3.45 (m, 2H), 2.06 (d, J=6.0 Hz, 2H), 1.95-1.78 (m, 3H), 1.66-1.65 (m, 5H), 1.59-1.46 (m, 2H), 1.42-1.33 (m, 1H), 1.18-1.17 (m, 2H) and LC-MS (ESI) found: 431 [M+H]⁺.

### Preparation of (2-((2R,3S,4S,5S,6R)-6-((7-(3-(hex-5-yn-1-yl) ureido)-9H-fluoren-2-yl)oxy)-3,4,5-trihydroxytetrahydro-2H-pyran-2-yl)ethyl)phosphonic acid (A83)

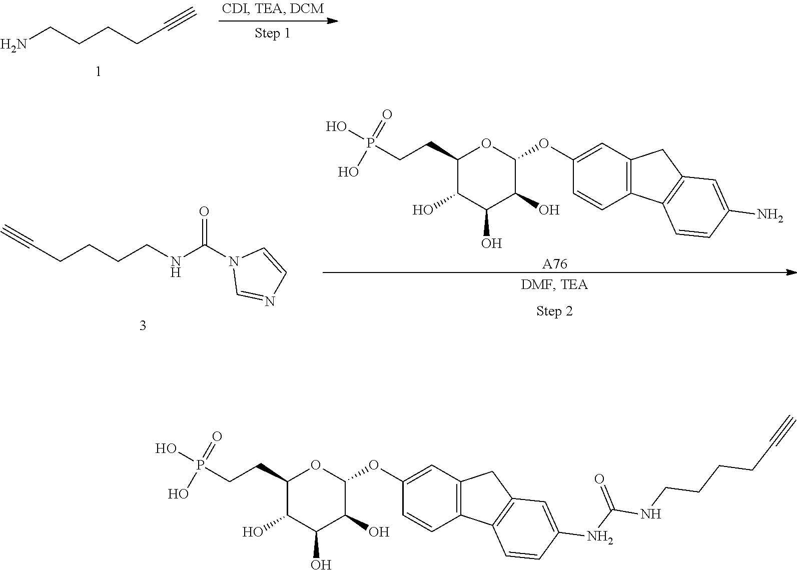

Step 1: To a solution of 1 (1.00 eq) in DCM (2 mL) were added Et₃N (1.50 eq) and CDI (1.30 eq) at 0° C. The mixture was stirred at 25° C. for 3 hours, then concentrated. The residue was purified by flash (DCM/MeOH=12:1) to afford 3 as yellow oil. Yield: 76%. LCMS found: [M+H]⁺=192. ¹H NMR (400 MHz, CDCl₃): δ 8.13 (s, 1H), 7.35 (s, 1H), 7.09 (s, 1H), 6.16 (brs, 1H), 3.50-3.45 (m, 2H), 2.29-2.25 (m, 2H), 2.00 (t, J=2.8 Hz, 1H), 1.80-1.75 (m, 2H), 1.65-1.61 (m, 2H).

Step 2: To a solution of 3 (1.20 eq) and A76 (1.00 eq) in DMF (0.5 mL) was added Et₃N (2.00 eq), the mixture was stirred at 70° C. for 1 hour. The mixture was dried by lyophilization to give crude product. The crude product was washed with MeCN (2 mL×5). The solid was dried in vacuo to afford the crude A83 (60% purity) as a light-yellow solid. Yield: 52%. LCMS found: [M−H]⁺=559. The crude product was then purified by prep-HPLC (10˜30% MeCN in H₂O, 0.1% FA). ¹H NMR (400 MHz, DMSO-d₆, 95% purity)δ 8.43 (s, 1H), 7.67 (s, 1H), 7.65 (d, J=8.4 Hz, 1H), 7.61 (d, J=8.0 Hz, 1H), 7.28 (dd, J=8.4, 1.6 Hz, 1H), 7.22 (d, J=2.0 Hz, 1H), 7.01 (dd, J=8.0, 2.0 Hz, 1H), 6.15 (t, J=5.6 Hz, 1H), 5.40 (d, J=1.2 Hz, 1H), 5.10-4.70 (m, 2H), 3.85 (dd, J=3.2, 1.6 Hz, 1H), 3.81 (s, 2H), 3.66 (dd, J=8.4, 3.2 Hz, 1H), 3.38-3.31 (m, 2H), 3.13-3.06 (m, 2H), 2.77 (t, J=2.4 Hz, 1H), 2.19 (td, J=6.8, 2.4 Hz, 2H), 2.03-1.88 (m, 1H), 1.65-1.46 (m, 6H), 1.23-1.13 (m, 1H). ³¹P NMR (162 MHz, CD₃OD) δ 26.41.

### Preparation of (2-((2R,3S,4S,5S,6R)-3,4,5-trihydroxy-6-(1-phenyl-1H-1,2,3-triazol-4-yl)tetrahydro-2H-pyran-2-yl)ethyl)phosphonic acid (A84)

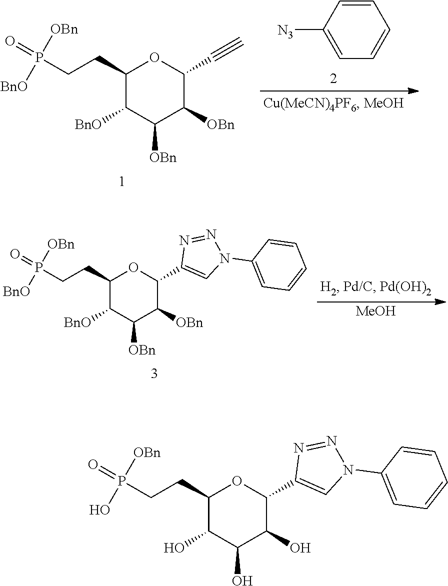 [SMILES: "MeOH" ; C#C[C@H]1O[C@H](CCP(=O)(OCc2ccccc2)OCc2ccccc2)[C@@H](OCc2ccccc2)[C@H](OCc2ccccc2)[C@@H]1OCc1ccccc1 ; [N-]=[N+]=Nc1ccccc1 ; O=P(CC[C@H]1O[C@H](c2cn(-c3ccccc3)nn2)[C@@H](OCc2ccccc2)[C@@H](OCc2ccccc2)[C@@H]1OCc1ccccc1)(OCc1ccccc1)OCc1ccccc1 ; O=P(O)(CC[C@H]1O[C@H](c2cn(-c3ccccc3)nn2)[C@@H](O)[C@@H](O)[C@@H]1O)OCc1ccccc1]

Step 1: To a solution of 1 (1.00 eq) and 2 (2.00 eq) in MeOH (2 mL) was added Cu(MeCN)₄PF₆ (2.00 eq) at 25° C., the mixture was stirred at 25° C. for 1 hour under N₂ atmosphere. LCMS showed the desired mass was detected. The mixture was concentrated and the residue was dissolved in EtOAc (10 mL), the organic layer was washed with saturated solution of NaHCO₃ and brine, dried with anhydrous Na₂SO₄, filtered and concentrated under reduced pressure. The crude product was purified by flash chromatography (silica gel, 0˜80% EA in PE) to give 3 as a yellow oil. Yield: 42.9%. LC-MS (ESI) found: 836 [M+H]⁺. Step 2: To a solution of 3 (1.00 eq) in MeOH (5 mL) was added Pd/C (10% purity) and Pd(OH)₂ (10% purity) at 25° C., the mixture was degassed and purged with hydrogen for 3 times, then stirred at 25° C. for 2 hours under hydrogen atmosphere (15 psi). LCMS showed the desired mass was detected. The mixture was filtered, the filtrate was concentrated in vacuo. The crude product was purified by prep-HPLC to give A84 as a white solid. Yield: 20.6%. LC-MS (ESI) found: 386 [M+H]⁺. ¹H NMR (400 MHz, CD₃OD): δ 8.55 (s, 1H), 7.87 (d, J=7.8 Hz, 2H), 7.59 (t, J=7.7 Hz, 2H), 7.50 (t, J=7.4 Hz, 1H), 5.16 (d, J=1.6 Hz, 1H), 4.52 (t, J=2.8 Hz, 1H), 3.84 (dd, J=8.7, 3.2 Hz, 1H), 3.62 (t, J=8.7 Hz, 1H), 3.46-3.38 (m, 1H), 2.20-2.09 (m, 1H), 2.05-1.84 (m, 2H), 1.81-1.68 (m, 1H).

### Preparation of (2-((2R,3S,4S,5S,6R)-6-(1-(1-(tert-butoxycarbonyl) piperidin-4-yl)-1H-1,2,3-triazol-4-yl)-3,4,5-trihydroxytetrahydro-2H-pyran-2-yl)ethyl)phosphonic acid (A85)

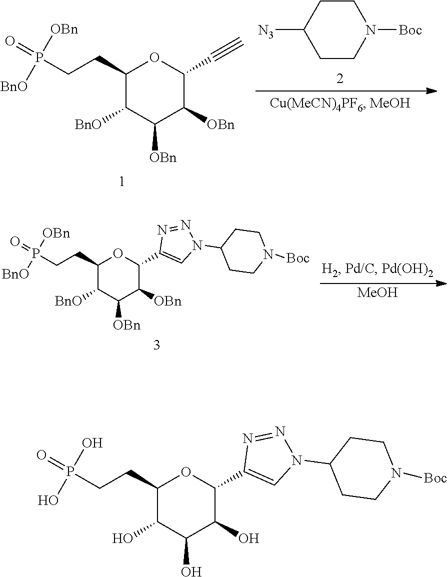

Step 1: To a solution of 1 (1.00 eq) and 2 (2.00 eq) in MeOH (2 mL) was added Cu(MeCN)₄PF₆ (3.00 eq) at 25° C., the mixture was stirred at 25° C. for 3 hours under N₂ atmosphere. LCMS showed the desired mass was detected. The mixture was concentrated and the residue was diluted in EtOAc (10 mL), the organic layer was washed with saturated solution of NaHCO₃ and brine, dried with anhydrous Na₂SO₄, filtered and concentrated under reduced pressure. The crude product was purified by flash chromatography (silica gel, 0˜60% EA in PE) to give 3 as a yellow oil. Yield: 57.0%. LC-MS (ESI) found: 943.5 [M+H]⁺.

Step 2: To a solution of 3 (1.00 eq) in MeOH (5 mL) was added Pd/C (10% purity) and Pd(OH)₂ (10% purity), the reaction was stirred at 25° C. for 2 hours under H₂ atmosphere (15 psi). LCMS showed the desired mass was detected. The mixture was filtered, the filtrate was concentrated in vacuo. The crude product was purified by prep-HPLC to give A85 as a white solid. Yield: 19.2%. LC-MS (ESI) found: 493 [M+H]⁺. ¹H NMR (400 MHz, CD₃OD): δ 8.06 (s, 1H), 5.07 (d, J=2.4 Hz, 1H), 4.76-4.69 (m, 1H), 4.44 (t, J=2.9 Hz, 1H), 4.26-4.17 (m, 2H), 3.74 (dd, J=8.8, 3.3 Hz, 1H), 3.57 (t, J=8.8 Hz, 1H), 3.30-3.26 (m, 1H), 3.06-2.96 (m, 2H), 2.20-2.09 (m, 3H), 2.04-1.93 (m, 3H), 1.90-1.82 (m, 1H), 1.77-1.66 (m, 1H), 1.48 (s, 9H).

### Preparation of (2-((2R,3S,4S,5S,6R)-3,4,5-trihydroxy-6-(1-(piperidin-4-yl)-1H-1,2,3-triazol-4-yl)tetrahydro-2H-pyran-2-yl)ethyl)phosphonic acid (A86)

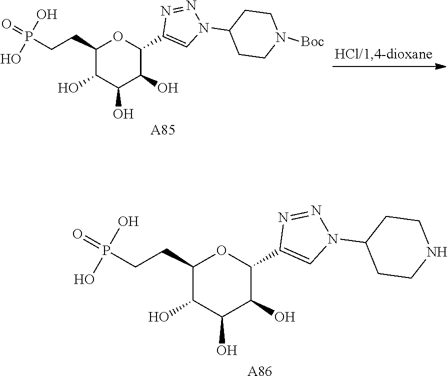 [SMILES: CC(C)(C)OC(=O)N1CCC(n2cc([C@H]3O[C@H](CCP(=O)(O)O)[C@@H](O)[C@H](O)[C@@H]3O)nn2)CC1 ; "A85" ; O=P(O)(O)CC[C@H]1O[C@H](c2cn(C3CCNCC3)nn2)[C@@H](O)[C@@H](O)[C@@H]1O ; "A86"]

The solution of A85 (1.00 eq) in HCl/1,4-dioxane (2 mL) was stirred at 25° C. for 1 hour. LCMS showed the desired mass was detected. The mixture was concentrated in vacuo. The crude product was purified by prep-HPLC to give A86 as a yellow solid. Yield: 92.1%. LCMS found: [M+H]⁺=393. ¹H NMR (400 MHz, CD₃OD): δ 8.17 (s, 1H), 5.09 (s, 1H), 4.44 (s, 1H), 3.74-3.67 (m, 1H), 3.63-3.53 (m, 4H), 3.42-3.35 (m, 1H), 3.27-3.22 (m, 2H), 2.47-2.30 (m, 4H), 1.99-1.71 (m, 4H).

### Synthesis of Peptide

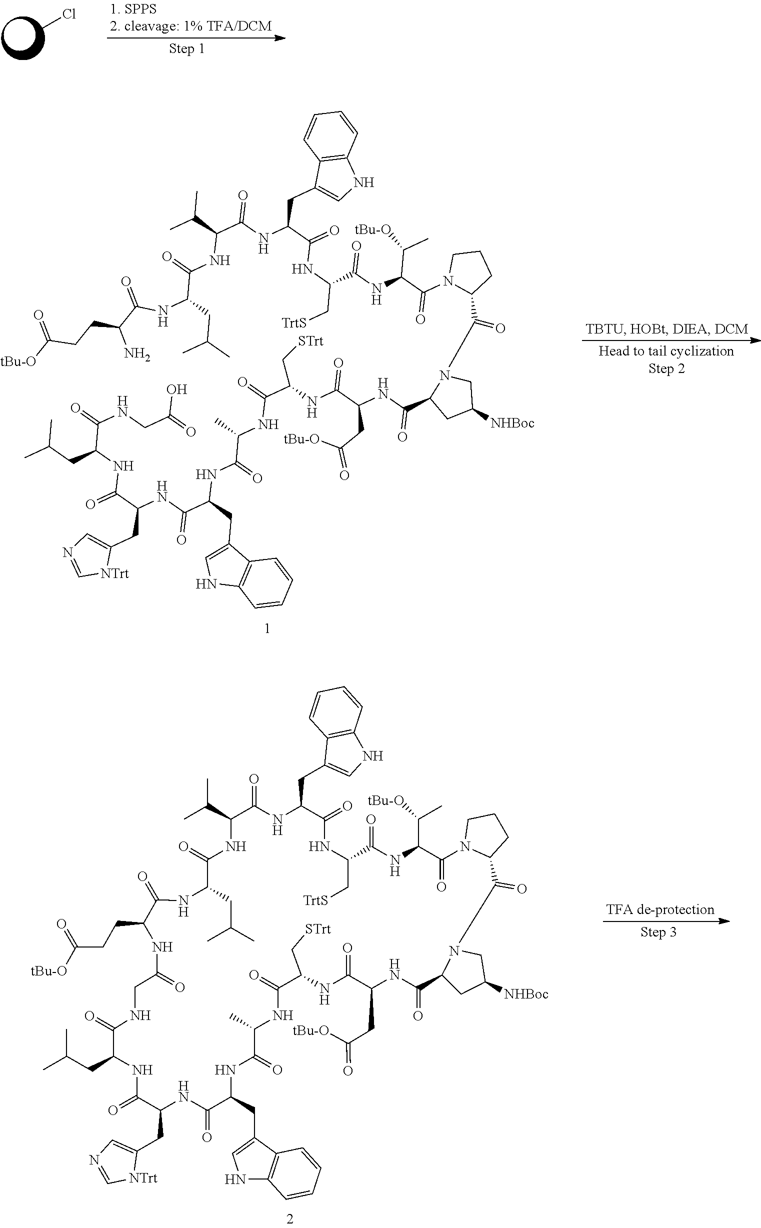

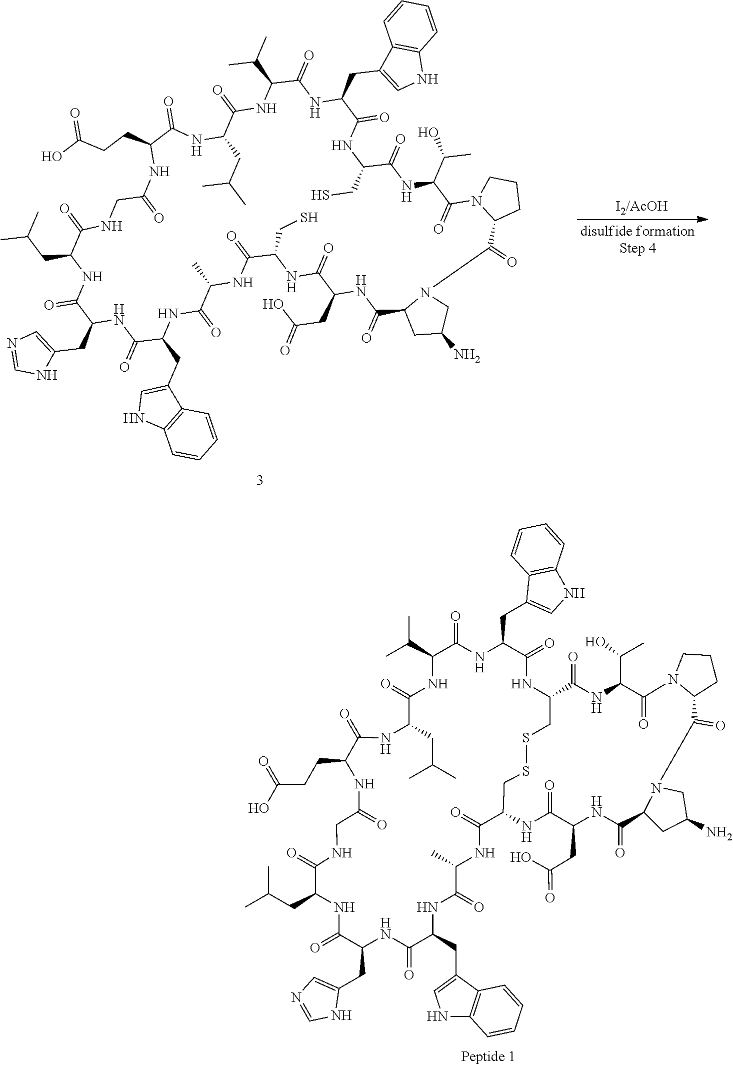

### Solid Phase Peptide Synthesis (SPPS)

The peptide was synthesized using standard Fmoc chemistry.
1)Resin preparation: To the vessel containing CTC Resin (100.00 g, 100.00 mmol, 1.00 mmol/g) and Fmoc-Gly-OH (29.70 g, 100.00 mmol, 1.00 eq) in DCM (1.5 L) was added DIEA (51.60 g, 69.73 mL, 400.00 mmol, 4.00 eq) dropwise and mixed for 2 hours with N₂ bubbling at 15° C. Then MeOH (100.00 mL) was added and the mixture was bubbled with N₂ for another 30 minutes. The resin was washed with DMF (3 L)*5. Then 20% piperidine in DMF (3 L) was added and the mixture was bubbled with N₂ for 30 minutes at 15° C. The mixture was filtered to obtain the resin. The resin was washed with DMF (3 L)*5 before proceeding to the next step.
2)Coupling: A solution of Fmoc-Leu-OH (105.90 g, 300.00 mmol, 3.00 eq), HBTU (108.15 g, 285.00 mmol, 2.85 eq) in DMF (1.5 L) was added to the resin with N₂ bubbling. Then DIEA (77.40 g, 104.58 mL, 600.00 mmol, 6.00 eq) was added to the mixture dropwise and bubbled with N₂ for 30 minutes at 15° C. The coupling reaction was monitored by ninhydrin test, if it showed colorless, the coupling was completed. The resin was then washed with DMF (3 L)*5.
3) De-protection: 20% piperidine in DMF (3 L) was added to the resin and the mixture was bubbled with N₂ for 30 minutes at 15° C. The resin was then washed with DMF (3 L)*5. The de-protection reaction was monitored by ninhydrin test, if it showed blue or other brownish red, the reaction was completed.
4)₂) and 3) were repeated for all other amino acids: Number #2-15 in Table 1.
5) After all steps were completed, the resin was washed with DMF (3 L)*5, MeOH (3 L)*5, then dried under reduced pressure to afford resin-bound peptide resin (100 mmol).

TABLE 1

| # | Materials | Coupling reagents |
|---|---|---|
| 1 | Fmoc-Gly-OH (3.00 eq) | HBTU (2.85 eq) and DIEA (6.00 eq) |
| 2 | Fmoc-Leu-OH (3.00 eq) | HBTU (2.85 eq) and DIEA (6.00 eq) |
| 3 | Fmoc-His(Trt)-OH (2.00 eq) | HBTU (2.85 eq) and DIEA (6.00 eq) |
| 4 | Fmoc-Trp-OH (3.00 eq) | HBTU (2.85 eq) and DIEA (6.00 eq) |
| 5 | Fmoc-Ala-OH (3.00 eq) | HBTU (2.85 eq) and DIEA (6.00 eq) |
| 6 | Fmoc-Cys(Trt)-OH (3.00 eq) | HBTU (2.85 eq) and DIEA (6.00 eq) |
| 7 | Fmoc-Asp(OtBu)-OH (3.00 eq) | HBTU (2.85 eq) and DIEA (6.00 eq) |
| 8 | Boc-(2S,4S)-4-amino-1-Fmoc- | HATU (1.42 eq) and DIEA (3.00 eq) |
| | pyrrolidine-2-carboxylic acid (1.50 eq) | |
| 9 | Fmoc-D-Pro-OH (1.00 eq) | DIEA (4.00 eq) |
| 10 | Fmoc-Thr(tBu)-OH (3.00 eq) | HBTU (2.85 eq) and DIEA (6.00 eq) |
| 11 | Fmoc-Cys(Trt)-OH (3.00 eq) | HBTU (2.85 eq) and DIEA (6.00 eq) |
| 12 | Fmoc-Trp-OH (3.00 eq) | HBTU (2.85 eq) and DIEA (6.00 eq) |
| 13 | Fmoc-Val-OH (3.00 eq) | HBTU (2.85 eq) and DIEA (6.00 eq) |
| 14 | Fmoc-Leu-OH (3.00 eq) | HBTU (2.85 eq) and DIEA (6.00 eq) |
| 15 | Fmoc-Glu(OtBu)-OH (3.00 eq) | HBTU (2.85 eq) and DIEA (6.00 eq) |

### Peptide Cleavage, Head to Tail Cyclization, TFA De-Protection, Disulfide Formation and Purification

1)Cleavage: Cleavage solution (1% TFA/DCM, v/v, 1.0 L) was added into the flask containing the side chain protected crude peptide resin (20 mmol). The mixture was stirred for 10 minutes at room temperature and the solution was collected after filtration. The cleavage step was repeated for another time. The combined solution afforded 1 (20 mmol) in cleavage solution.
2) Head to tail cyclization: The combined solution (1, 20 mmol) was diluted to 20 L with DCM. Then TBTU (12.84 g, 40.00 mmol, 2.00 eq), and HOBt (5.40 mg, 40.00 mmol, 2.00 eq) were added and the mixture was basified using DIEA to pH=8 at 25° C. The reaction mixture was stirred at 15° C. for 2 hours. LCMS indicated one main peak with the desired MS. Then the mixture was washed with 1M HCl (5 L), H₂O (5 L), brine (5 L), dried over anhydrous Na₂SO₄, filtered and concentrated under vacuum to afford 2 (38.10 g, crude) as a white solid. Chemical Formula: C₁₅₃H₁₈₄N₂₀₀₂₂S2, LCMS found: [M+2H]²⁺=1360.77;
3) TFA de-protection: A mixture of 2 (38.10 g, crude) in TFA/TIS/H₂O/3-mercaptopropanoic acid (92.5/2.5/2.5/2.5, v/v/v/v, 400 mL) was stirred at 15° C. for 1.5 hours. The mixture was precipitated with cold isopropyl ether (2 L). After filtration, the solid was dried under vacuum for 2 hours to afford 3 (28.40 g, crude, TFA salt) as a white solid.
4) Disulfide formation: 3 (28.40 g, crude, TFA salt) was dissolved in MeCN/H₂O (1/1, v/v, 70 L). 0.1 M I₂/HOAc was added to the mixture dropwise at 15° C. until the light yellow persisted, then the mixture was quenched with 0.1 M Na₂S203 dropwise until the light yellow disappeared.
5) Purification: After filtration, the filtrate was purified by prep-HPLC (acid condition, TFA) directly and followed by lyophilization to afford Peptide 1 (6.25 g, 18.1% yield, 88.3% purity, TFA salt) as a white solid. Chemical Formula: C₇₉H₁₀₈N₂₀₀₂₀S2, LCMS found: [M+H]⁺=1722.8, [M+2H]²⁺=861.6.
6)Repeat step 1-5 above, total 4 batches (20 mmol*4) were performed to afford total Peptide 1 (25.00 g, TFA salt) as a white solid.

### Synthesis of Heterobifunctional Compounds

### Preparation of Compound 1:3-((2S,5aR,11S,14R,17S,20S,23S,26S,32S,35S,38S, 41S,44R,47S,49aS)-35-((1H-imidazol-5-yl)methyl)-17,38-bis((1H-indol-3-yl)methyl)-47-(carboxymethyl)-2-(24-(4-((3-((hydroxy (2-((2R,3S,4S,5S)-3,4,5-trihydroxy-6-(4-methoxyphenoxy)tetrahydro-2H-pyran-2-yl)ethyl)phosphoryl)methyl) phenoxy)methyl)-1H-1,2,3-triazol-1-yl)-13,13-bis((3-((2-(2-(4-((3-((hydroxy (2-((2S,3R,4R,5R)-3,4,5-trihydroxy-6-(4-methoxyphenoxy)tetrahydro-2H-pyran-2-yl)ethyl)phosphoryl)methyl) phenoxy)methyl)-1H-1,2,3-triazol-1-yl)ethoxy)ethyl)amino)-3-oxopropoxy)methyl)-11,18-dioxo-3,6,9,15,22-pentaoxa-12,19-diazatetracosanamido)-11-((R)-1-hydroxyethyl)-23,32-diisobutyl-20-isopropyl-41-methyl-5,10,13,16,19,22,25,28,31,34,37,40,43,46,49-pentadecaoxooctatetracontahydro-5H-14,44-(methanodithiomethano)dipyrrolo[1,2-a:1′,2′-d][1,4,7,10,13,16,19,22,25,28,31,34,37,40,43]pentadecaazacyclopentatetracontin-26-yl)propanoic acid

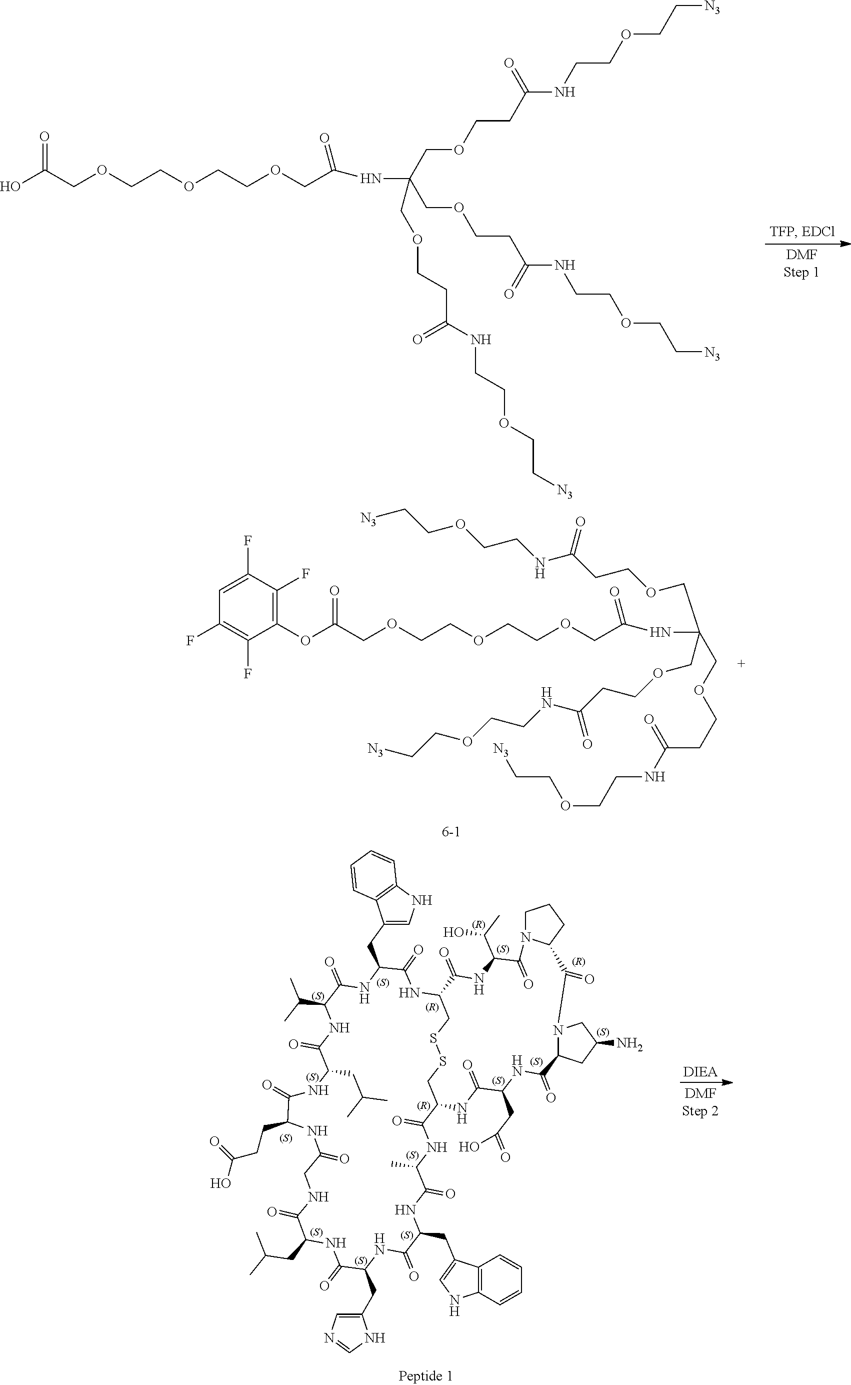

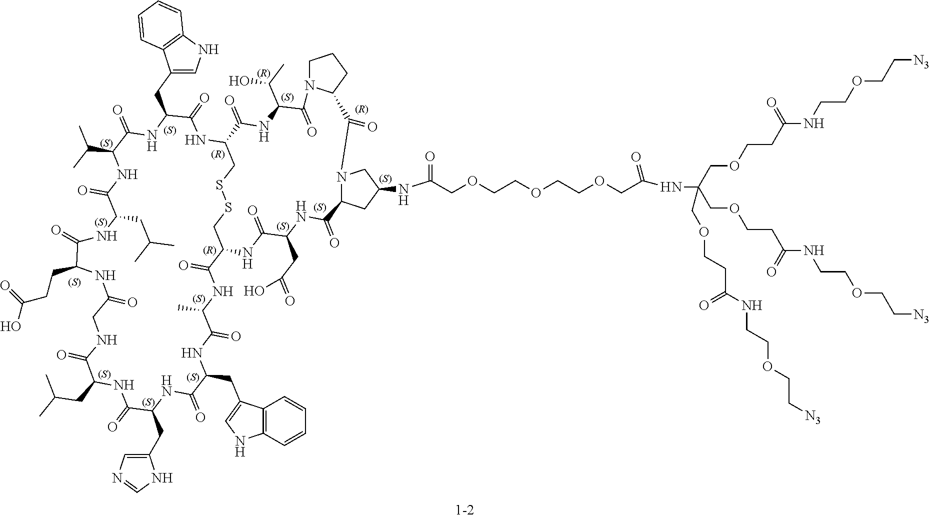

### Step 1

To a mixture of 24-azido-13,13-bis((3-((2-(2-azidoethoxy)ethyl)amino)-3-oxopropoxy)methyl)-11,18-dioxo-3,6,9,15,22-pentaoxa-12,19-diazatetracosanoic acid (70.0 mg, 79.7 μmol, 1.0 eq) and tetrafluorophenol (19.8 mg, 120 μmol, 1.5 eq) in DMF (1.0 mL) was added EDCI (22.9 mg, 120 μmol, 1.5 eq) and HOBt (16.1 mg, 120 μmol, 1.5 eq) at 20° C. The reaction was stirred at 20° C. for 1 hour. The mixture was purified by prep-HPLC (TFA condition) directly to afford compound 1-1 (22.0 mg, 26.9% yield) as colorless oil. Chemical Formula: C₃₉H₅₉F4N₁₃₀₁₅. LCMS found: [M+H]⁺=1026.5, [M+2H]²⁺=513.8.

### Step 2

To a mixture of 1-1 (22.0 mg, 22.4 μmol, 1.0 eq) and Peptide 1 (77.2 mg, 44.9 μmol, 2.0 eq) in DMF (1 mL) was added DIEA (5.70 mg, 44.9 μmol, 74.0 μL 2.0 eq) at 20° C. The reaction was stirred at 20° C. for 1 hour. The mixture was purified by prep-HPLC (TFA condition) directly to afford 1-2 (44.7 mg, 69.5% yield) as a white solid. Chemical Formula: C₁₁₂H₁₆₅N₃₃O₃₄S2. LCMS found: [M+2H]²⁺=1291.4, [M+3H]³⁺=861.3.

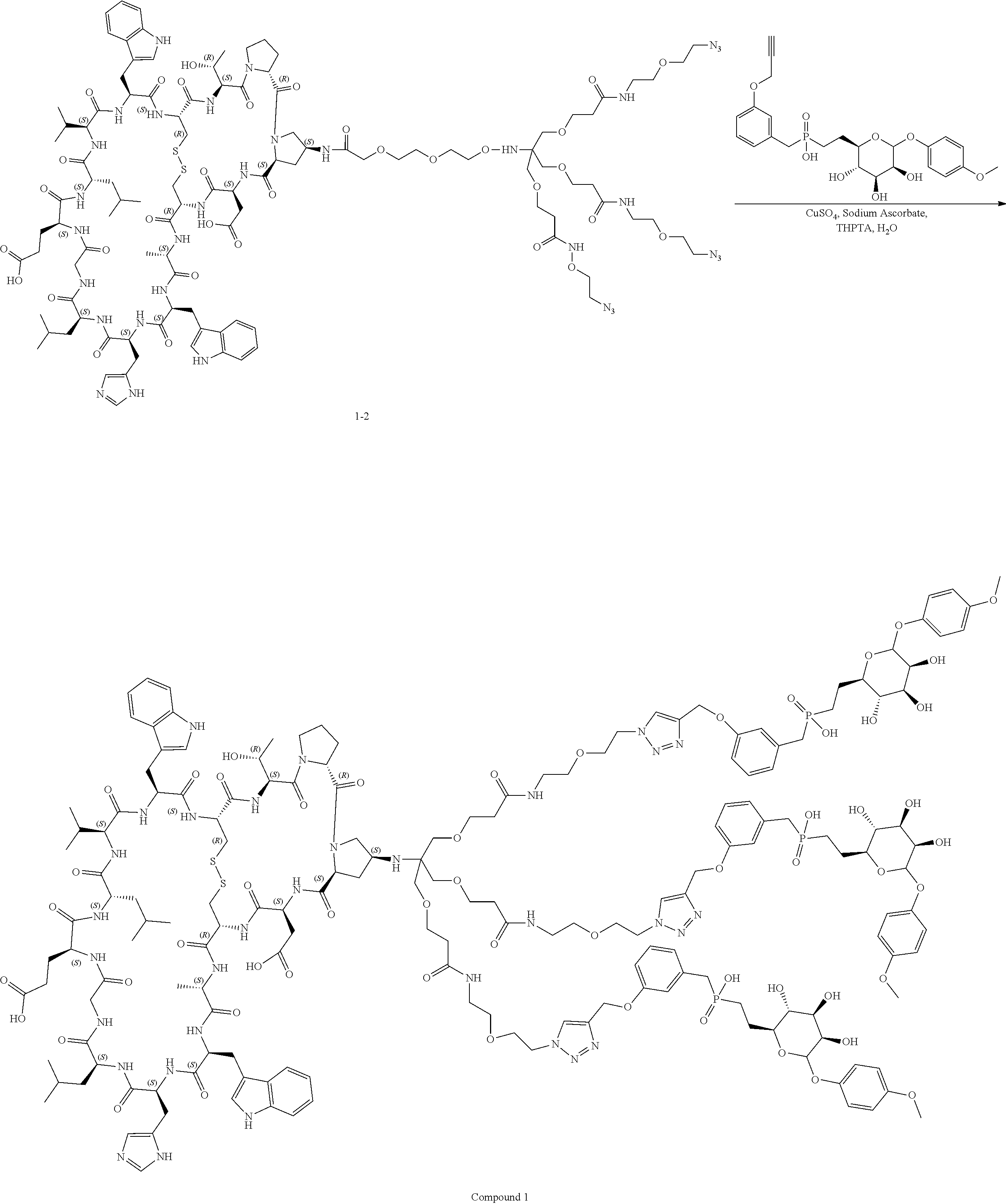

To a mixture of 1-2 (10.0 mg, 3.87 μmol, 1.0 eq) and (3-(prop-2-yn-1-yloxy)benzyl) (2-((2R,3S,4S,5S)-3,4,5-trihydroxy-6-(4-methoxyphenoxy)tetrahydro-2H-pyran-2-yl)ethyl)phosphinic acid (7.60 mg, 15.5 μmol, 4.0 eq) in DMF (0.2 mL) was added was added a freshly prepared solution of CuSO₄ (0.4 M, 38.7 μL, 15.5 μmol, 4.0 eq) in H₂O, sodium ascorbate (6.90 mg, 34.9 μmol, 9.0 eq), and THPTA (6.86 mg, 15.5 μmol, 4.0 eq) under N₂ at 0° C. The mixture was warmed up to 20° C. and stirred at 20° C. for 2 hours. LCMS revealed one major peak with desired mass. The crude was purified by prep-HPLC (TFA condition) directly to afford Compound 1 (10.0 mg, 61.9% yield) as a white solid. Chemical Formula: C184H₂₅₂N₃₃O₆₁P3S2. LCMS found: [M+3H]³⁺=1354.0, [M+4H]4+=1015.9.

Compound 2, Compound 3, and Compound 4 were synthesized similarly as described for Compound 1.

| ID | Starting material | Analytical data |
|---|---|---|
| Compound 2 | 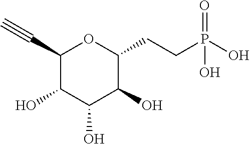 | Yield: 13.3 mg, 97.4% purity, 49.5% yield, white solid. LC-MS found: [M + 2H]²⁺ = 1691.1, [M + 3H]³⁺ = 1127.7, [M + 4H]⁴⁺ = 846.2. |
| | A20 | |
| Compound 3 | 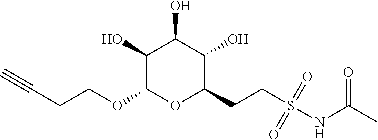 | Yield: 1.2 mg with 95.2% purity, 8.1% yield, white solid. LC-MS found: [M + 2H]²⁺ = 1819.1, [M + 3H]³⁺ = 1212.9, [M + 4H]⁴⁺ = 909.9. |
| Compound 4 | 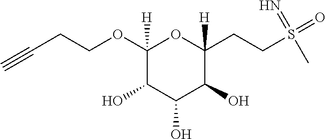 | Yield: 7.0 mg with 99.3% purity, 42.4% yield, white solid. LC-MS found: [M + 2H]²⁺ = 1752.8, [M + 3H]³⁺ = 1168.6, [M + 4H]⁴⁺ = 877.0. |

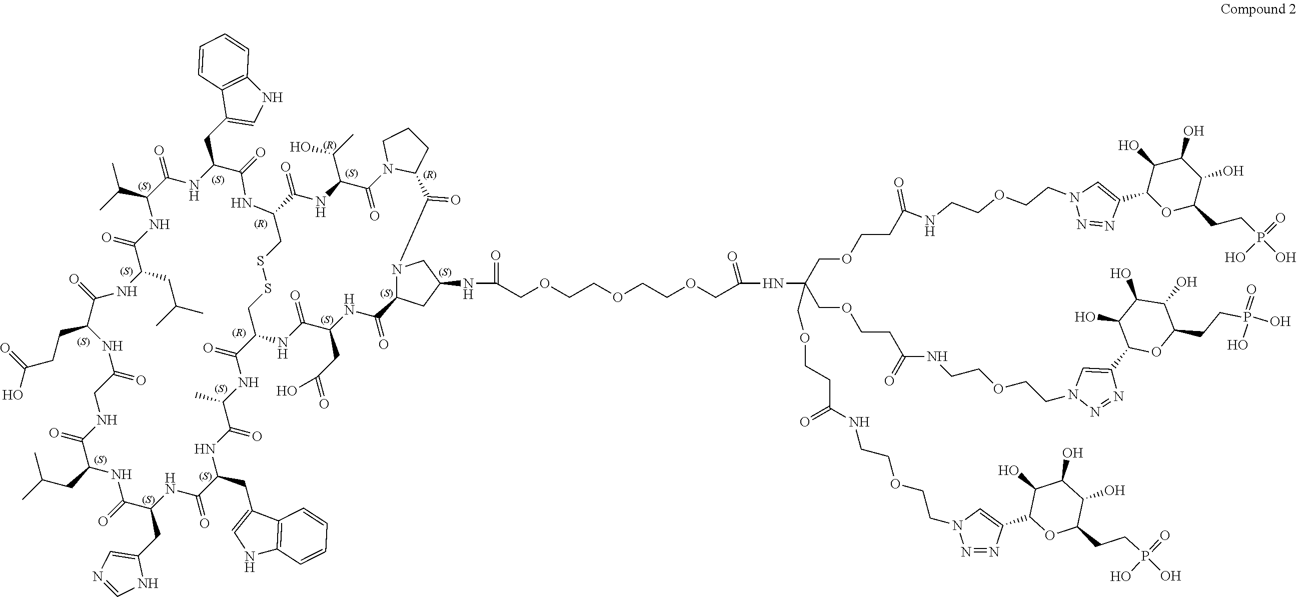

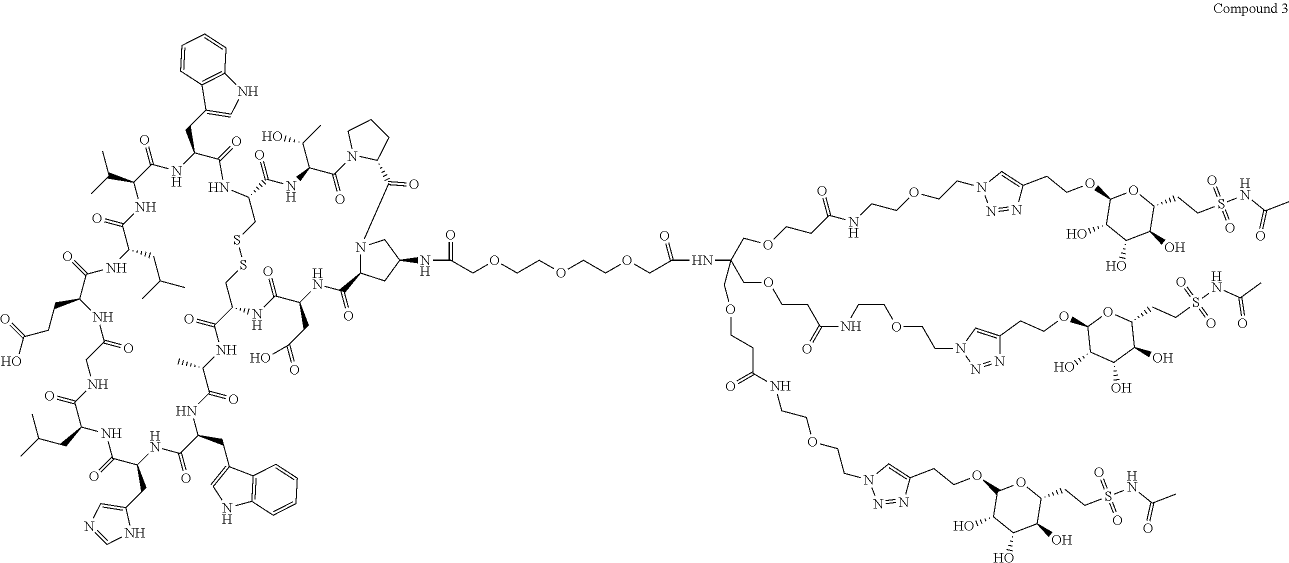

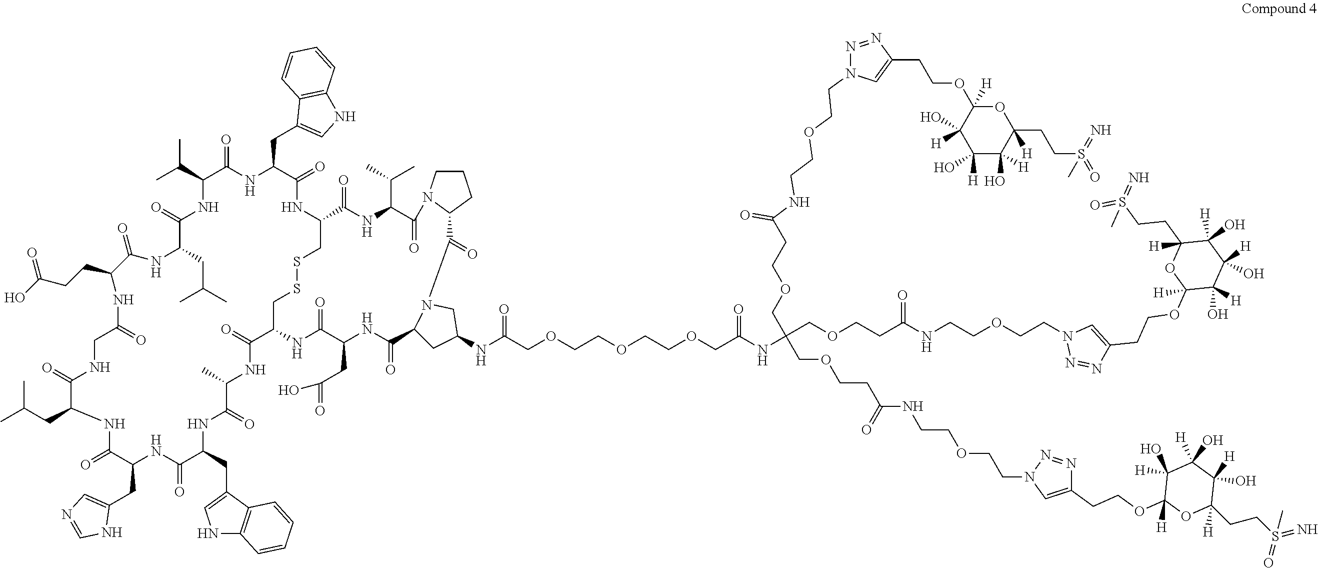

### Preparation of Compound 5:3-((6S,9R,12S,15S,18S,21S,27S,30S,33S,36S,39R,42S,44aS,46S,49aR)-30-((1H-imidazol-5-yl)methyl)-12,33-bis((1H-indol-3-yl)methyl)-42-(carboxymethyl)-46-(11,18-dioxo-13,13-bis((3-oxo-3-((2-(2-(4-(2-(((2S,3S,4S,5S,6R)-3,4,5-trihydroxy-6-(2-phosphonoethyl)tetrahydro-2H-pyran-2-yl)oxy)ethyl)-1H-1,2,3-triazol-1-yl)ethoxy)ethyl)amino) propoxy)methyl)-24-(4-(2-(((2S,3S,4S,5S,6R)-3,4,5-trihydroxy-6-(2-phosphonoethyl)tetrahydro-2H-pyran-2-yl)oxy)ethyl)-1H-1,2,3-triazol-1-yl)-3,6,9,15,22-pentaoxa-12,19-diazatetracosanamido)-6-((R)-1-hydroxyethyl)-18,27-diisobutyl-15-isopropyl-36-methyl-5,8,11,14,17,20,23,26,29,32,35,38,41,44,49-pentadecaoxooctatetracontahydro-1H-9,39-(methanodithiomethano)dipyrrolo[1,2-a:1′,2′-d][1,4,7,10,13,16,19,22,25,28,31,34,37,40,43]pentadecaazacyclopentatetracontin-21-yl)propanoic acid

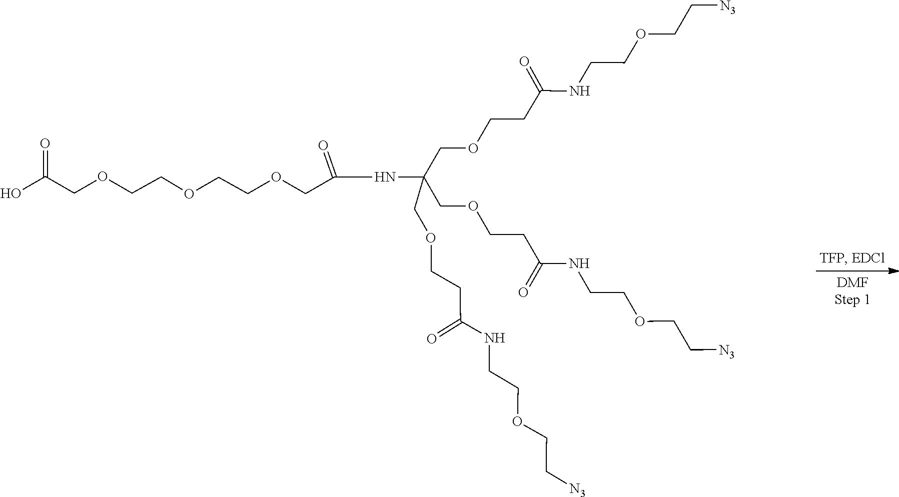 [SMILES: [N-]=[N+]=NCCOCCNC(=O)CCOCC(COCCC(=O)NCCOCCN=[N+]=[N-])(COCCC(=O)NCCOCCN=[N+]=[N-])NC(=O)COCCOCCOCC(=O)O]

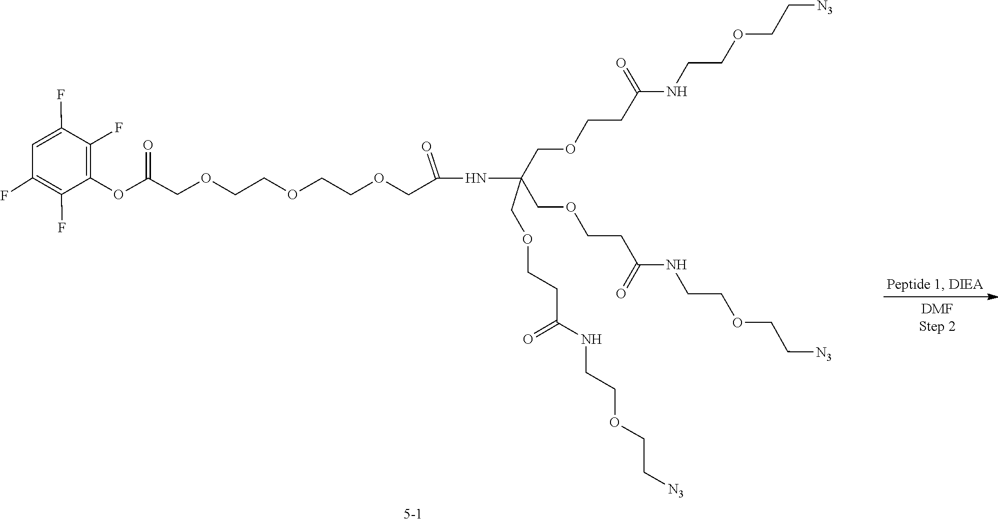 [SMILES: [N-]=[N+]=NCCOCCNC(=O)CCOCC(COCCC(=O)NCCOCCN=[N+]=[N-])(COCCC(=O)NCCOCCN=[N+]=[N-])NC(=O)COCCOCCOCC(=O)Oc1c(F)c(F)cc(F)c1F]

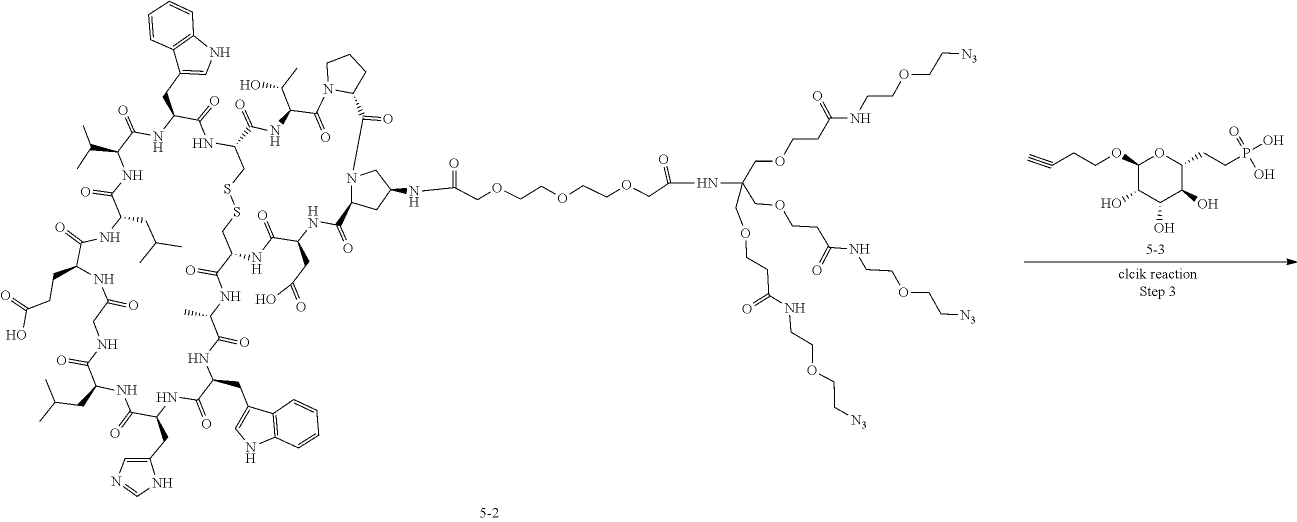 [SMILES: CC(C)C[C@@H]1NC(=O)[C@H](Cc2cnc[nH]2)NC(=O)[C@H](Cc2c[nH]c3ccccc23)NC(=O)[C@H](C)NC(=O)[C@@H]2CSSC[C@H](NC(=O)[C@H](Cc3c[nH]c4ccccc34)NC(=O)[C@H](C(C)C)NC(=O)[C@H](CC(C)C)NC(=O)[C@H](CCC(=O)O)NC(=O)CNC1=O)C(=O)N[C@@H]([C@@H](C)O)C(=O)N1CCC[C@@H]1C(=O)N1C[C@@H](NC(=O)COCCOCCOCC(=O)NC(COCCC(=O)NCCOCCN=[N+]=[N-])(COCCC(=O)NCCOCCN=[N+]=[N-])COCCC(=O)NCCOCCN=[N+]=[N-])C[C@H]1C(=O)N[C@@H](CC(=O)O)C(=O)N2 ; C#CCCO[C@H]1O[C@H](CCP(=O)(O)O)[C@@H](O)[C@H](O)[C@@H]1O]

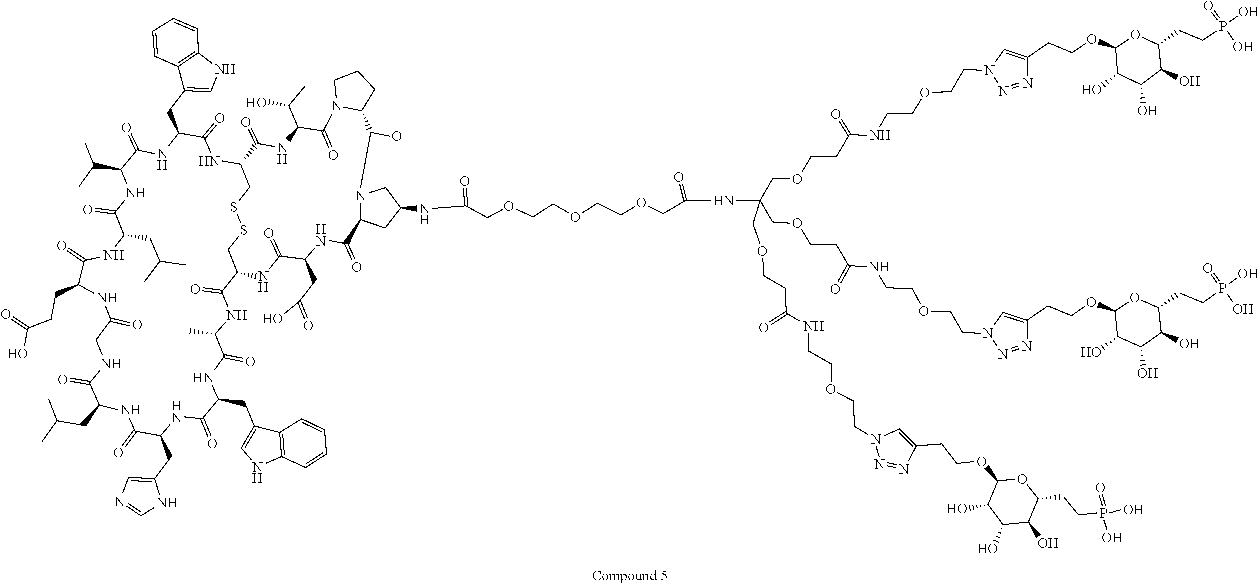

### Step 1: Preparation of TFP Ester (5-1)

To a mixture of 24-azido-13,13-bis((3-((2-(2-azidoethoxy)ethyl)amino)-3-oxopropoxy)methyl)-11,18-dioxo-3,6,9,15,22-pentaoxa-12,19-diazatetracosanoic acid (110.0 mg, 125.30 μmol, 1.00 eq), 2,3,5,6-tetrafluorophenol (124.8 mg, 751.80 μmol, 6.00 eq) in DMF (1 mL) was added EDCI (72.0 mg, 375.90 μmol, 3.00 eq) at 0° C. Then the mixture was stirred at 0° C. for 3 hours. The reaction mixture was purified by prep-HPLC (acid condition, TFA) directly followed by lyophilization to afford 5-1 (TFP ester, 61.0 mg, 47.4% yield, 90% purity) as a white solid. Chemical Formula: C₃₉H₅₉F4N₁₃₀₁₅, LCMS found: [M+H]⁺=1026.50, [M+Na]⁺=1048.40,.

### Step 2: Preparation of 5-2

A mixture of 5-1 (61.0 mg, 59.46 μmol, 1.00 eq), Peptide 1 (112.62 mg, 65.40 μmol, 1.10 eq), DIEA (38.42 mg, 297.28 μmol, 51.78 μL, 5.00 eq) in DMF (1 mL) was stirred at 20° C. for 3 hours. The reaction mixture was cooled to room temperature, acidified by 1 M HCl to pH=5, purified by prep-HPLC (acid condition, TFA) directly and followed by lyophilization to afford compound (83.0 mg, 54.0% yield, 90.0% purity) as a white solid. Chemical Formula: C₁₁₂H₁₆₅N₃₃O₃₄S2, LCMS found: [M+2H]²⁺=1292.79, [M+3H]³⁺=861.5.

### Step 3: Preparation of Compound 5

A mixture of 5-2 (83.0 mg, 32.15 μmol, 1.00 eq), 5-3 (34.9 mg, 112.52 μmol, 3.50 eq) in DMF (1 mL) was cooled to 0° C. Then a fresh solution of CuSO₄ (0.4 M, 241.77 μL, 3.00 eq), sodium ascorbate (0.4 M, 725.31 μL, 9.00 eq), 3-[4-[[bis [[1-(3-hydroxypropyl)triazol-4-yl]methyl]amino]methyl]triazol-1-yl]propan-1-ol (41.9 mg, 96.45 μmol, 3.00 eq) was added to the mixture and stirred at 0° C. for 1 hour under N₂ atmosphere. Then the mixture was purified by prep-HPLC (acid condition, TFA) directly and followed by lyophilization to afford Compound 5 (52.0 mg, 42.96% yield, 93.3% purity) as a white solid. Chemical Formula: C₁₄₅H₂₂₂N₃₃O₅₈P3S2, LCMS found: [M+2H]²⁺=1765.7, [M+2H+Na]³⁺=1177.9, [M+3H]³⁺=1171.5.

### Preparation of Compound 6:23,32-diisobutyl-20-isopropyl-41-methyl-5,10,13,16,19,22,25,28,31,34,37,40,43,46,49-pentadecaoxo-2-(1-(4-(2-(((2S,3S,4S,5S,6R)-3,4,5-trihydroxy-6-(2-phosphonoethyl)tetrahydro-2H-pyran-2-yl)oxy)ethyl)-1H-1,2,3-triazol-1-yl)-3,6,9,12,15,18,21,24-octaoxaheptacosan-27-amido)octatetracontahydro-5H-14,44-(methanodithiomethano)dipyrrolo[1,2-a:1′,2′-d][1,4,7,10,13,16,19,22,25,28,31,34,37,40,43]pentadecaazacyclopentatetracontin-26-yl)propanoic acid

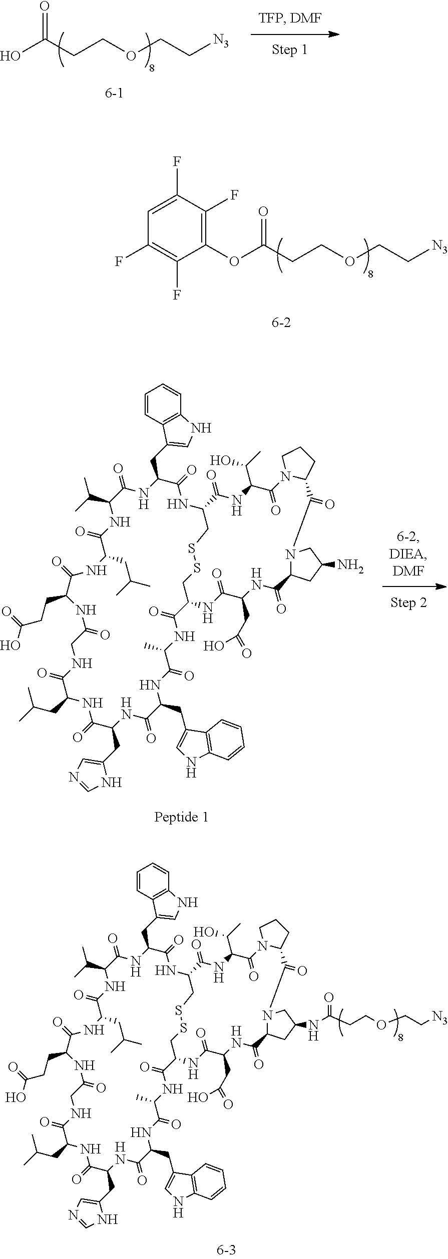

### Step 1: Preparation of Compound 6-3

To a mixture of 6-1 (370 mg, 0.79 mmol, 1.0 eq) and tetrafluorophenol (197 mg, 1.19 mmol, 1.5 eq) in DMF (5.0 mL) was added EDCI (227 mg, 1.19 mmol, 1.5 eq) and HOBt (161 mg, 1.19 mmol, 1.5 eq) at 20° C. The reaction was stirred at 20° C. for 1 hour. The mixture was purified by prep-HPLC (TFA condition) directly to afford 6-2 (445 mg, 91.5% yield) as colorless oil. Chemical Formula: C₂₅H₃₇F₄N₃O₁₀. LCMS found: [M+H]⁺=616.3 [M+2H]+=308.1. To a mixture of 6-2 (445 mg, 0.73 mmol, 1.0 eq) and Peptide 1 (2.51 g, 1.46 mmol, 2.0 eq) in DMF (20 mL) was added DIEA (118 mg, 1.46 mmol, 2.0 eq) at 20° C. The reaction was stirred at 20 10° C. for 1 hr. The mixture was purified by prep-HPLC (TFA condition) directly to afford 6-3 (640 mg, 40.4% yield) as a white solid. Chemical Formula: C₉₈H₁₄₃N₂₃O₂₉S2. LCMS found: [M+2H]2+=1086.4, [M+3H]³⁺=715.5.

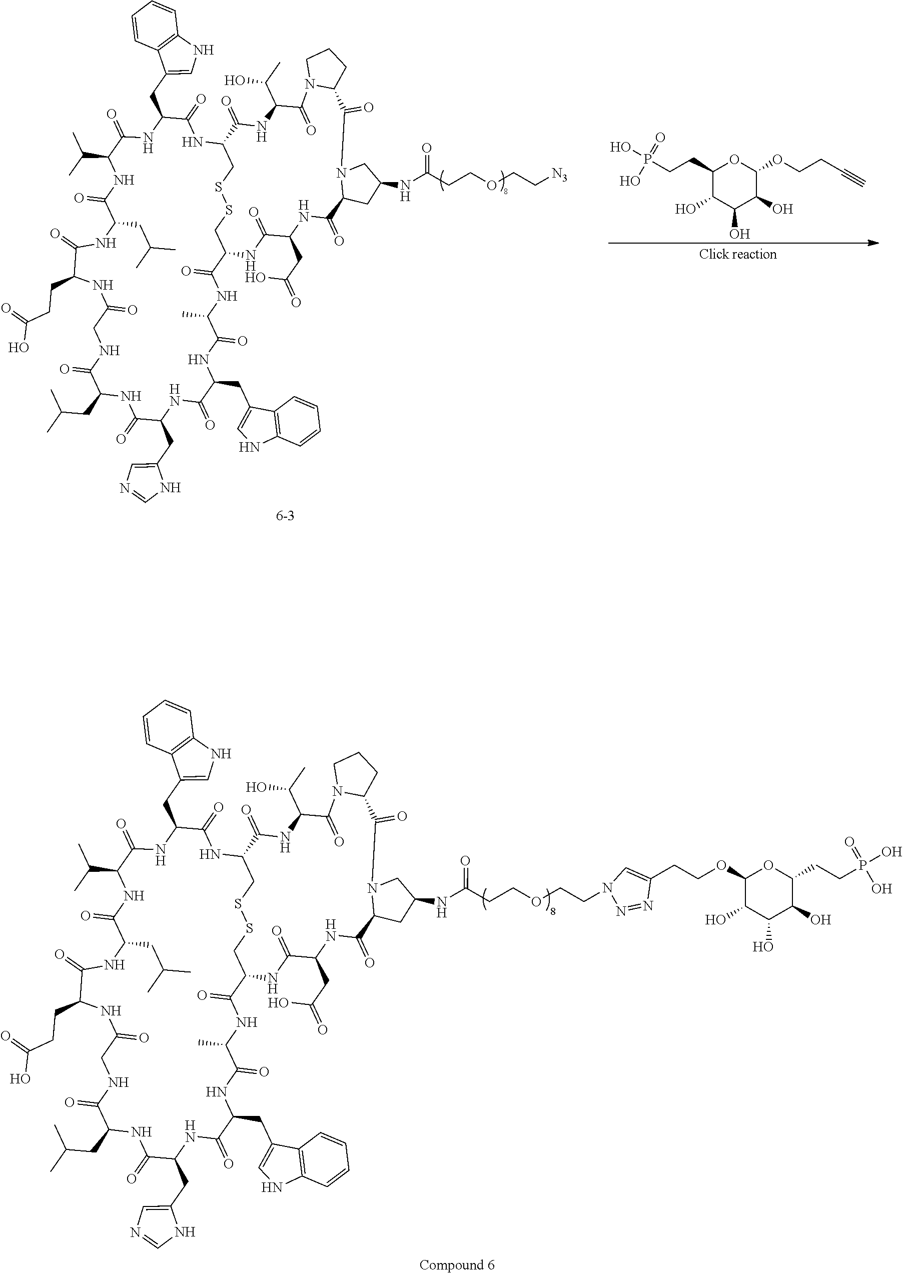

To a mixture of 6-3 (90.0 mg, 41.5 μmol, 1.0 eq) and (2-((2R,3S,4S,5S,6S)-6-(but-3-yn-1-yloxy)-3,4,5-trihydroxytetrahydro-2H-pyran-2-yl)ethyl)phosphonic acid (15.4 mg, 49.7 μmol, 1.2 eq) in DMF (1.0 mL) was added a freshly prepared solution of CuSO₄ (0.4 M, 104 μL, 41.5 μmol, 1.0 eq) in H₂O, sodium ascorbate (32.8 mg, 166 μmol, 4.0 eq), and THPTA (18.4 mg, 41.5 μmol, 1.0 eq) under N₂ at 0° C. The mixture was warmed up to 20° C. and stirred at 20° C. for 2 hours. LCMS revealed one major peak with desired mass. The crude was purified by prep-HPLC (TFA condition) directly to afford Compound 6 (48.5 mg, 44.9% yield) as a white solid. Chemical Formula: C₁₀₉H₁₆₂N₂₃O₃₇PS₂. LCMS found: [M+2H]²⁺=1241.9, [M+3H]³⁺=828.2.

Compound 7 and Compound 8 were synthesized similarly as described for Compound 6.

| ID | Starting material | Analytical data |
|---|---|---|
| Compound 7 | 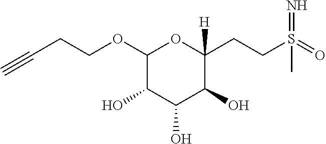 | Yield: 3.5 mg, 96.8% purity, 30.7% yield, white solid. LC-MS found: [M + 2H]²⁺ = 1240.0, [M + 3H]³⁺ = 826.9. |
| | A33 | |

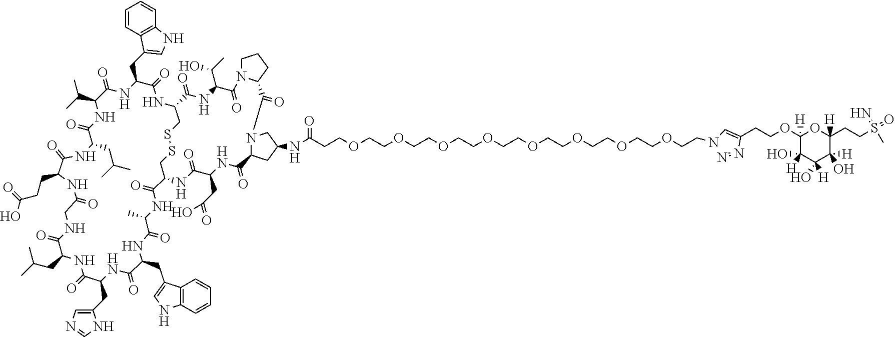 [SMILES: CC(C)C[C@@H]1NC(=O)[C@H](Cc2cnc[nH]2)NC(=O)[C@H](Cc2c[nH]c3ccccc23)NC(=O)[C@H](C)NC(=O)[C@@H]2CSSC[C@H](NC(=O)[C@H](Cc3c[nH]c4ccccc34)NC(=O)[C@H](C(C)C)NC(=O)[C@H](CC(C)C)NC(=O)[C@H](CCC(=O)O)NC(=O)CNC1=O)C(=O)N[C@@H]([C@@H](C)O)C(=O)N1CCC[C@@H]1C(=O)N1C[C@@H](NC(=O)CCOCCOCCOCCOCCOCCOCCOCCOCCn3cc(CCO[C@H]4O[C@H](CCS(C)(=N)=O)[C@@H](O)[C@H](O)[C@@H]4O)nn3)C[C@H]1C(=O)N[C@@H](CC(=O)O)C(=O)N2]

Compound 7

| | | |
|---|---|---|
| Compound 8 | 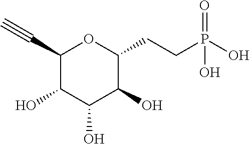 | Yield: 11.5 mg, 99.0% purity, 51.3% yield, white solid. LC-MS found: [M + 2H]²⁺ = 1219.4, [M + 3H]³⁺ = 813.4. |
| | A20 | |

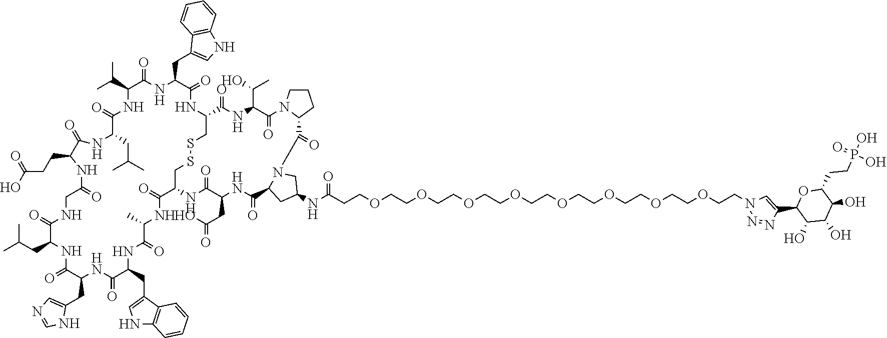 [SMILES: CC(C)C[C@@H]1NC(=O)[C@H](Cc2cnc[nH]2)NC(=O)[C@H](Cc2c[nH]c3ccccc23)NC(=O)[C@H](C)NC(=O)[C@@H]2CSSC[C@H](NC(=O)[C@H](Cc3c[nH]c4ccccc34)NC(=O)[C@H](C(C)C)NC(=O)[C@H](CC(C)C)NC(=O)[C@H](CCC(=O)O)NC(=O)CNC1=O)C(=O)N[C@@H]([C@@H](C)O)C(=O)N1CCC[C@@H]1C(=O)N1C[C@@H](NC(=O)CCOCCOCCOCCOCCOCCOCCOCCOCCn3cc([C@H]4O[C@H](CCP(=O)(O)O)[C@@H](O)[C@H](O)[C@@H]4O)nn3)C[C@H]1C(=O)N[C@@H](CC(=O)O)C(=O)N2]

Compound 8

### Preparation of Compound 9:3-((6S,9R,12S,15S,18S,21S,27S,30S,33S,36S,39R,42S,44aS,46S,49aR)-30-((1H-imidazol-5-yl)methyl)-12,33-bis((1H-indol-3-yl)methyl)-42-(carboxymethyl)-6-((R)-1-hydroxyethyl)-18,27-diisobutyl-15-isopropyl-36-methyl-5,8,11,14,17,20,23,26,29,32,35,38,41,44,49-pentadecaoxo-46-(1-(4-(2-(((2S,3S,4S,5S,6R)-3,4,5-trihydroxy-6-(2-phosphonoethyl)tetrahydro-2H-pyran-2-yl)oxy)ethyl)-1H-1,2,3-triazol-1-yl)-12-(2-(2-(2-(2-(4-(2-(((2S,3S,4S,5S,6R)-3,4,5-trihydroxy-6-(2-phosphonoethyl)tetrahydro-2H-pyran-2-yl)oxy)ethyl)-1H-1,2,3-triazol-1-yl)ethoxy)ethoxy)ethoxy)ethyl)-3,6,9,15,18,21-hexaoxa-12-azatetracosanamido)octatetracontahydro-1H-9,39-(methanodithiomethano)dipyrrolo[1,2-a:1′,2′-d][1,4,7,10,13,16,19,22,25,28,31,34,37,40,43]pentadecaazacyclopentatetracontin-21-yl)propanoic acid

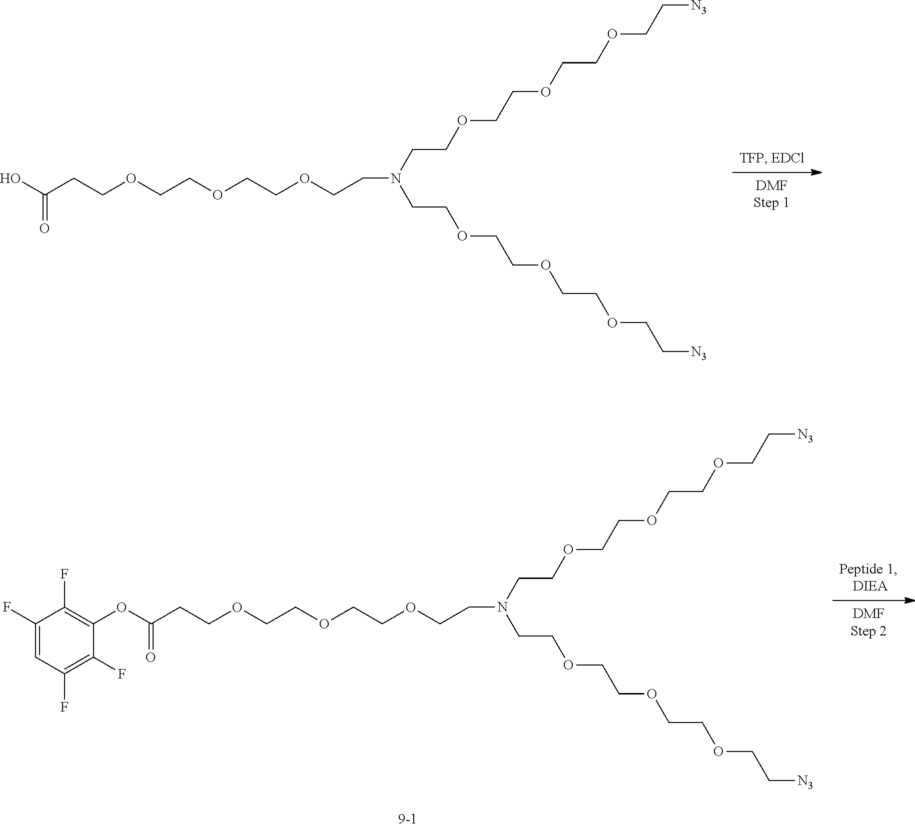 [SMILES: [N-]=[N+]=NCCOCCOCCOCCN(CCOCCOCCOCCN=[N+]=[N-])CCOCCOCCOCCC(=O)O ; [N-]=[N+]=NCCOCCOCCOCCN(CCOCCOCCOCCN=[N+]=[N-])CCOCCOCCOCCC(=O)Oc1c(F)c(F)cc(F)c1F]

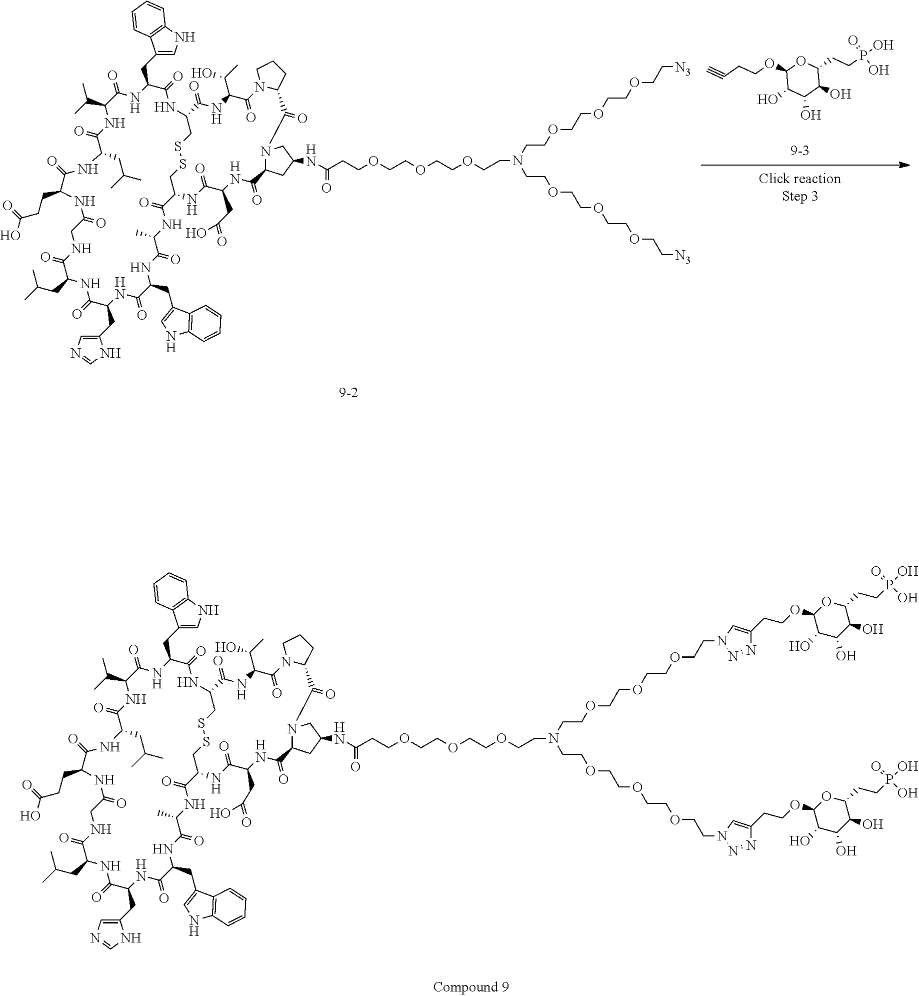

### Step 1: Preparation of TFP Ester (9-1)

To a mixture of 1-azido-12-(2-(2-(2-(2-azidoethoxy)ethoxy)ethoxy)ethyl)-3,6,9,15,18,21-hexaoxa-12-azatetracosan-24-oic acid (80.0 mg, 128.27 μmol, 1.00 eq), 2,3,5,6-tetrafluorophenol (127.81 mg, 769.61 μmol, 6.00 eq) in DMF (0.5 mL) was added EDCI (73.77 mg, 384.80 μmol, 2.50 eq) at 0° C. Then the mixture was stirred at 0° C. for 3 hours. The reaction mixture was purified by prep-HPLC (acid condition, TFA) directly and followed by lyophilization to afford 9-1 (TFP ester, 21.00 mg, 21.2% yield, 95.0% purity) as a white solid. Chemical Formula: C₃₁H₄₉F₄N₇₀₁₁, LCMS found: [M+H]⁺=772.48, [M+Na]⁺=794.40,.

### Step 2: Preparation of 9-2

A mixture of 9-1 (21.0 mg, 27.21 μmol, 1.00 eq), Peptide 1 (51.5 mg, 29.93 μmol, 1.10 eq), DIEA (14.0 mg, 108.84 μmol, 18.96 μL, 4.00 eq) in DMF (0.8 mL) was stirred at 20° C. for 3 hours. The reaction mixture was cooled to room temperature, acidified by 1 M HCl to pH=5, purified by prep-HPLC (acid condition, TFA) directly followed by lyophilization to afford 9-2 (34.5 mg, 54.4% yield, 95.0% purity) as a white solid. Chemical Formula: C₁₀₄H₁₅₅N₂₇O₃₀S2, LCMS found: [M+2H]²⁺=1164.40, [M+3H]³⁺=776.71

### Step 3: Preparation of Compound 9

A mixture of 9-2 (34.5 mg, 14.82 μmol, 1.00 eq), (2-((2R,3S,4S,5S,6S)-6-(but-3-yn-1-yloxy)-3,4,5-trihydroxytetrahydro-2H-pyran-2-yl)ethyl)phosphonic acid 9-3 (11.5 mg, 37.05 μmol, 2.50 eq) in DMF (0.5 mL) was cooled to 0° C. Then a fresh solution of CuSO₄ (0.4 M, 74.11 μL, 2.00 eq), sodium ascorbate (0.4 M, 222.33 μL, 6.00 eq), 3-[4-[[bis [[1-(3-hydroxypropyl)triazol-4-yl]methyl]amino]methyl]triazol-1-yl]propan-1-ol (12.8 mg, 29.64 μmol, 2.00 eq) was added to the mixture and stirred at 0° C. for 1 hour under N₂ atmosphere. Then the mixture was purified by prep-HPLC (acid condition, TFA) directly and followed by lyophilization to afford Compound 9 (23.5 mg, 52.7% yield, 98.0% purity) as a white solid. Chemical Formula: Cl₂₆H₁₉₃N₂₇O₄₆P₂S2, LCMS found: [M+2H]²⁺=1474.50, [M+3H]³⁺=983.90.

### Preparation of Compound 10:3-((2S,5aR,11S,14R,17S,20S,23S,26S,32S,35S,38S,41S,44R,47S,49aS)-35-((1H-imidazol-5-yl)methyl)-17,38-bis((1H-indol-3-yl)methyl)-47-(carboxymethyl)-2-(24-(4-((hydroxy (2-((2R,3S,4S,5S)-3,4,5-trihydroxy-6-(4-methoxyphenoxy)tetrahydro-2H-pyran-2-yl)ethyl)phosphoryl)methyl)-1H-1,2,3-triazol-1-yl)-13-((3-((2-(2-(4-((hydroxy (2-((2R,3S,4S,5S)-3,4,5-trihydroxy-6-(4-methoxyphenoxy)tetrahydro-2H-pyran-2-yl)ethyl)phosphoryl)methyl)-1H-1,2,3-triazol-1-yl)ethoxy)ethyl)amino)-3-oxopropoxy)methyl)-13-((3-((2-(2-(4-((hydroxy (2-((2S,3R,4R,5R)-3,4,5-trihydroxy-6-(4-methoxyphenoxy)tetrahydro-2H-pyran-2-yl)ethyl)phosphoryl)methyl)-1H-1,2,3-triazol-1-yl)ethoxy)ethyl)amino)-3-oxopropoxy)methyl)-11,18-dioxo-3,6,9,15,22-pentaoxa-12,19-diazatetracosanamido)-11-((R)-1-hydroxyethyl)-23,32-diisobutyl-20-isopropyl-41-methyl-5,10,13,16,19,22,25,28,31,34,37,40,43,46,49-pentadecaoxooctatetracontahydro-5H-14,44-(methanodithiomethano)dipyrrolo[1,2-a:1′,2′-d][1,4,7,10,13,16,19,22,25,28,31,34,37,40,43]pentadecaazacyclopentatetracontin-26-yl)propanoic acid

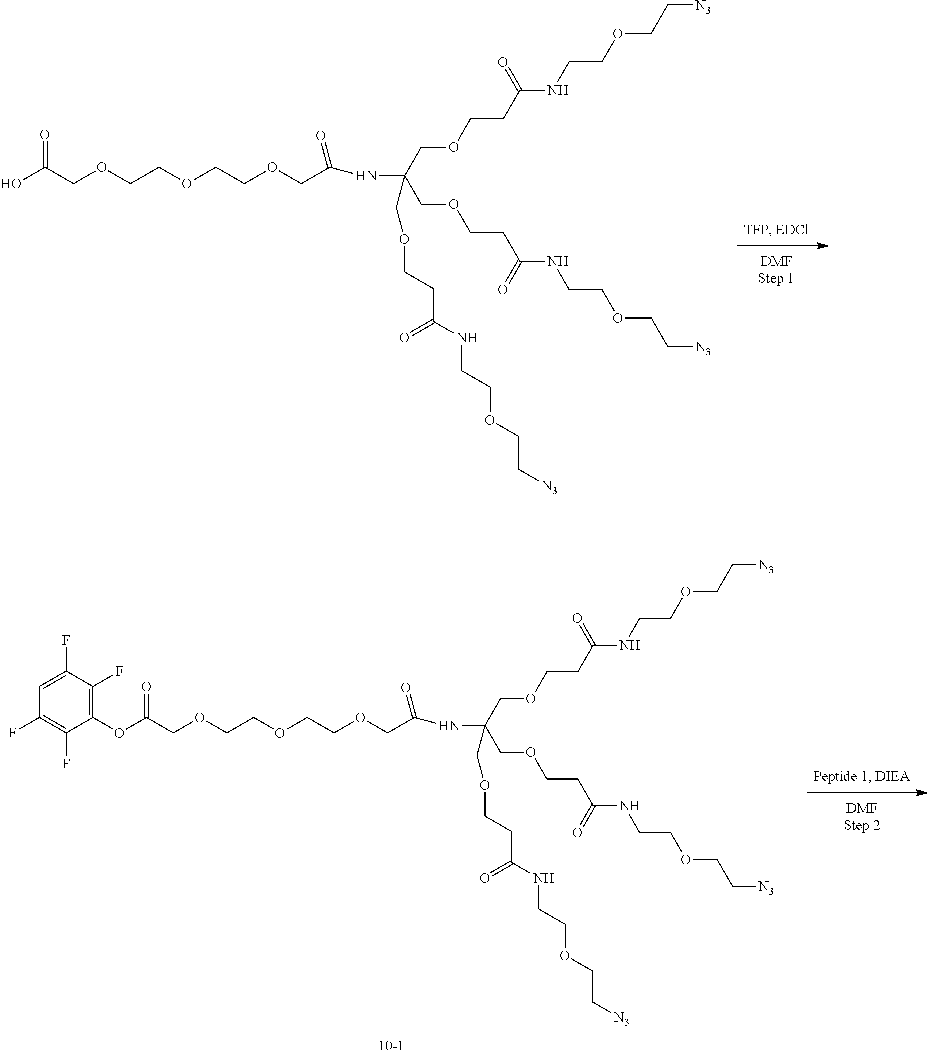 [SMILES: [N-]=[N+]=NCCOCCNC(=O)CCOCC(COCCC(=O)NCCOCCN=[N+]=[N-])(COCCC(=O)NCCOCCN=[N+]=[N-])NC(=O)COCCOCCOCC(=O)O ; [N-]=[N+]=NCCOCCNC(=O)CCOCC(COCCC(=O)NCCOCCN=[N+]=[N-])(COCCC(=O)NCCOCCN=[N+]=[N-])NC(=O)COCCOCCOCC(=O)Oc1c(F)c(F)cc(F)c1F]

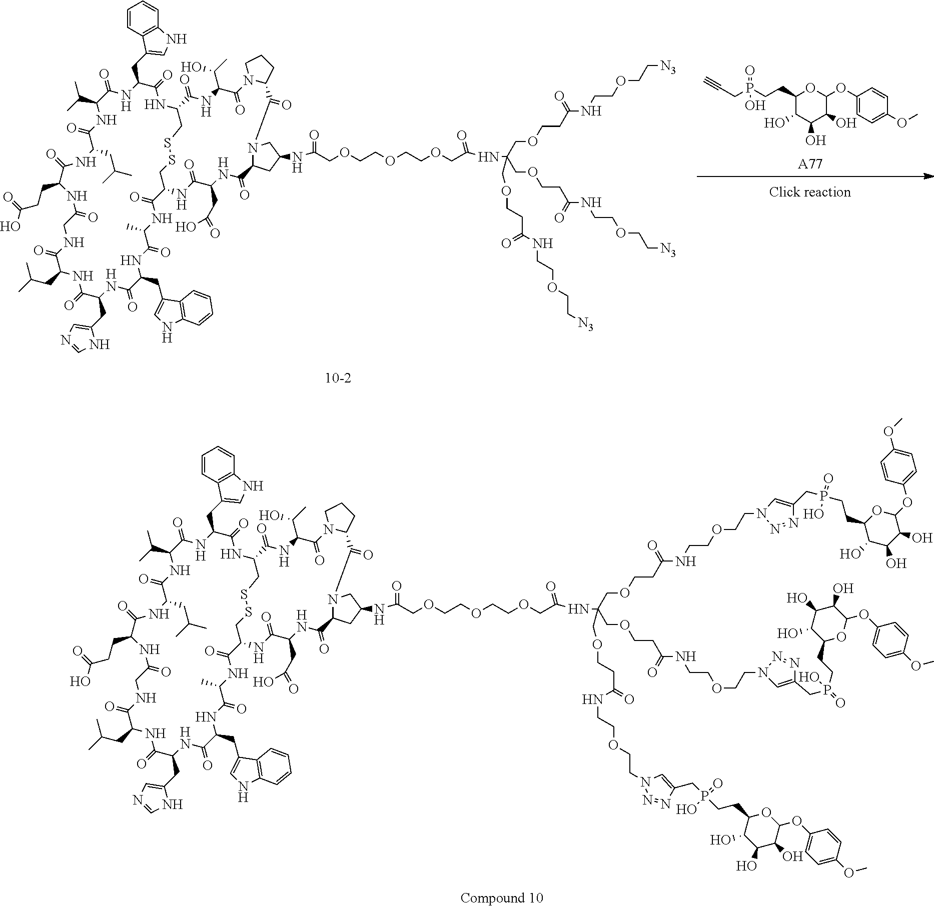

### Step 1: Preparation of 10-1

To a mixture of 24-azido-13,13-bis((3-((2-(2-azidoethoxy)ethyl)amino)-3-oxopropoxy)methyl)-11,18-dioxo-3,6,9,15,22-pentaoxa-12,19-diazatetracosanoic acid (70.0 mg, 79.7 μmol, 1.0 eq) and tetrafluorophenol (19.8 mg, 120 μmol, 1.5 eq) in DMF (1.0 mL) was added EDCI (22.9 mg, 120 μmol, 1.5 eq) and HOBt (16.1 mg, 120 μmol, 1.5 eq) at 20° C. The reaction was stirred at 20° C. for 1 hour. The mixture was purified by prep-HPLC (TFA condition) directly to afford 10-1 (22.0 mg, 26.9% yield) as colorless oil. Chemical Formula: C₃₉H₅₉F₄N₁₃₀₁₅. LCMS found: [M+H]⁺=1026.5, [M+2H]²⁺=513.8.

### Step 2: Preparation of 10-2

To a mixture of 10-1 (22.0 mg, 22.4 μmol, 1.0 eq) and Peptide 1 (77.2 mg, 44.9 μmol, 2.0 eq) in DMF (1 mL) was added DIEA (5.70 mg, 44.9 μmol, 74.0 μL 2.0 eq) at 20° C. The reaction was stirred at 20° C. for 1 hour. The mixture was purified by prep-HPLC (TFA condition) directly to afford 10-2 (44.7 mg, 69.5% yield) as a white solid. Chemical Formula: C₁₁₂H₁₆₅N₃₃O₃₄S2. LCMS found: [M+2H]²⁺=1291.4, [M+3H]³⁺=861.3.

### Step 3: Preparation of Compound 10

To a mixture of 10-2 (10.0 mg, 3.87 μmol, 1.0 eq) and A77 (5.99 mg, 15.5 μmol, 4.0 eq) in DMF (0.2 mL) was added a freshly prepared solution of CuSO₄ (0.4 M, 38.7 μL, 15.5 μmol, 4.0 eq) in H₂O, sodium ascorbate (6.90 mg, 34.9 μmol, 9.0 eq), and THPTA (6.86 mg, 15.5 μmol, 4.0 eq) under N₂ at 0° C. The mixture was warmed up to 20° C. and stirred at 20° C. for 2 hours. LCMS revealed one major peak with desired mass. The crude was purified by prep-HPLC (TFA condition) directly to afford Compound 10 (3.7 mg, 24.5% yield 95.2% purity) as a white solid. Chemical Formula: C₁₆₃H₂₃₄N₃₃O₅₈P₃S2. Molecular Weight: 3740.84. LCMS found: [M+3H]³⁺=1247.9, [M+2H]²⁺=1871.8.

### Preparation of Compound 11:3-((2S,5aR,11R,14R,17S,20R,23R,26S,32R,35S,38S,41S,44R,47S,49aS)-35-((1H-imidazol-5-yl)methyl)-17,38-bis((1H-indol-3-yl)methyl)-47-(carboxymethyl)-2-(1-(4-((hydroxy (2-((2R,3S,4S,5S)-3,4,5-trihydroxy-6-(4-methoxyphenoxy)tetrahydro-2H-pyran-2-yl)ethyl)phosphoryl)methyl)-1H-1,2,3-triazol-1-yl)-3,6,9,12,15,18,21,24-octaoxaheptacosan-27-amido)-11-((R)-1-hydroxyethyl)-23,32-diisobutyl-20-isopropyl-41-methyl-5,10,13,16,19,22,25,28,31,34,37,40,43,46,49-pentadecaoxooctatetracontahydro-5H-14,44-(methanodithiomethano)dipyrrolo[1,2-a:1′,2′-d][1,4,7,10,13,16,19,22,25,28,31,34,37,40,43]pentadecaazacyclopentatetracontin-26-yl)propanoic acid

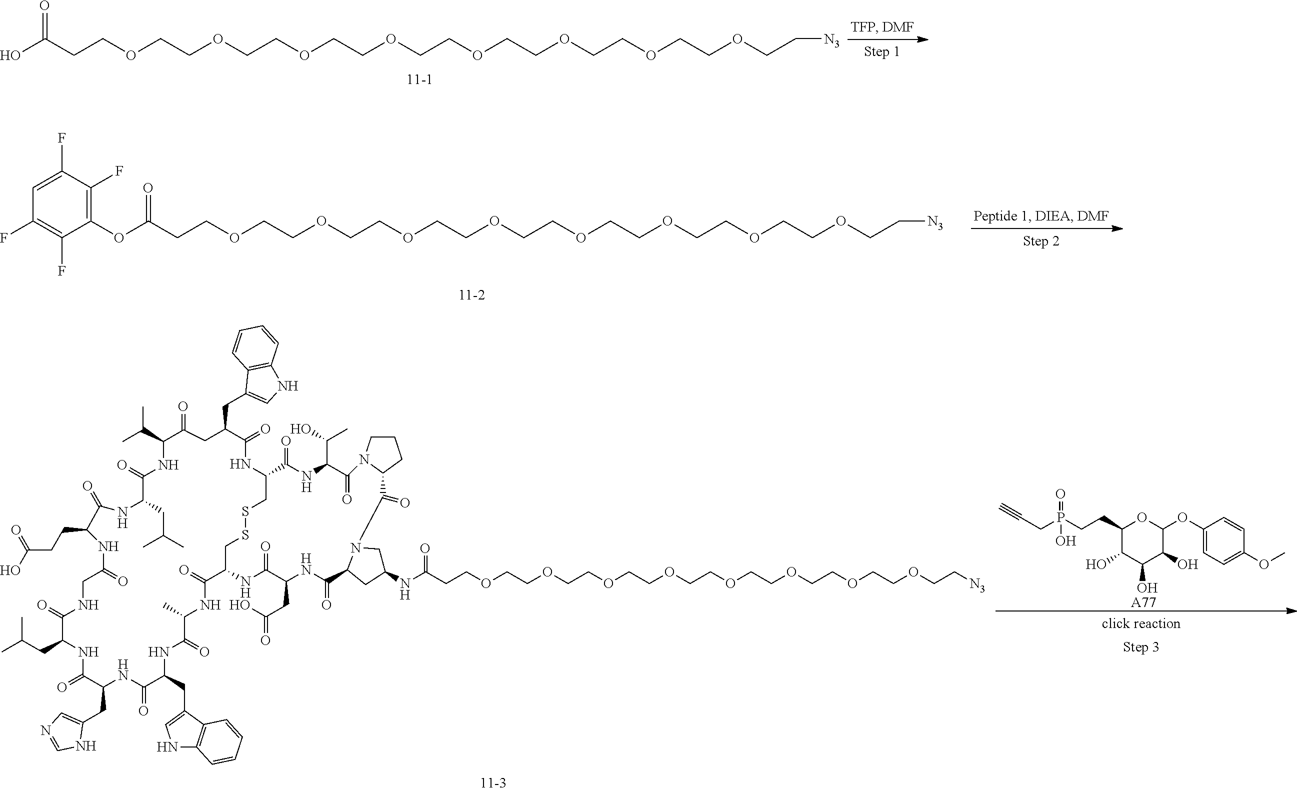

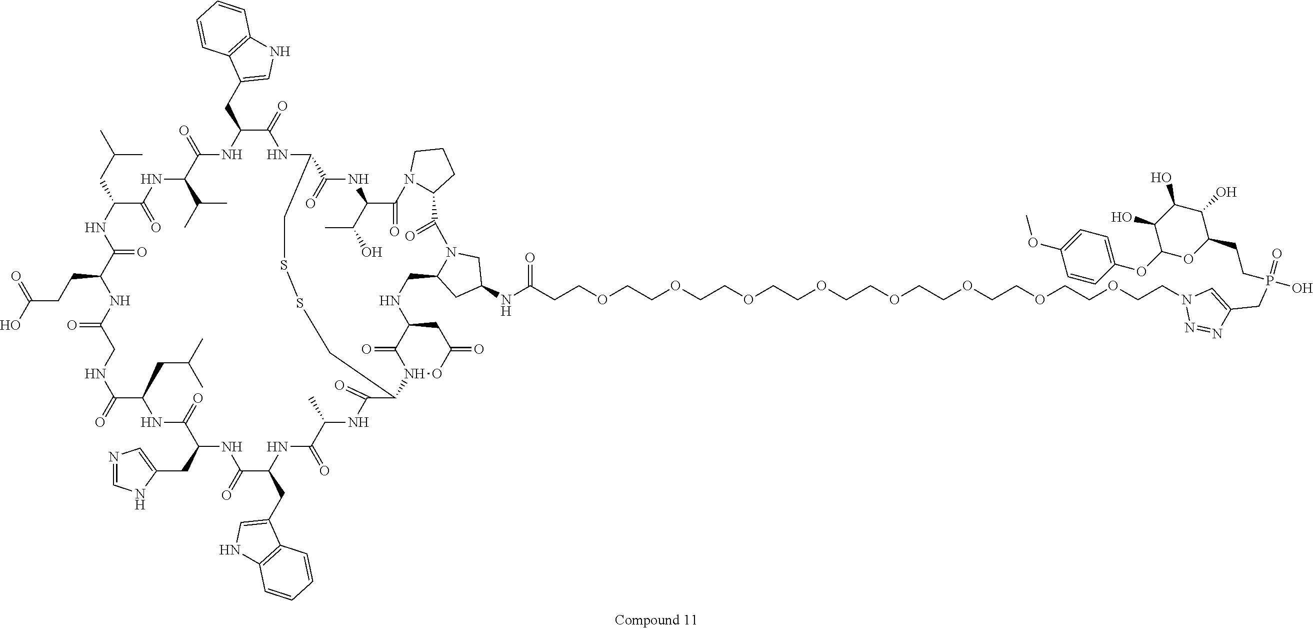 [SMILES: COc1ccc(OC2O[C@H](CCP(=O)(O)Cc3cn(CCOCCOCCOCCOCCOCCOCCOCCOCCC(=O)N[C@H]4C[C@H]5CN[C@H]6CC(=O)ON(C6=O)[C@H]6CSSC[C@H](NC(=O)[C@H](Cc7c[nH]c8ccccc78)NC(=O)[C@@H](C(C)C)NC(=O)[C@@H](CC(C)C)NC(=O)[C@H](CCC(=O)O)NC(=O)CNC(=O)[C@@H](CC(C)C)NC(=O)[C@H](Cc7cnc[nH]7)NC(=O)[C@H](Cc7c[nH]c8ccccc78)NC(=O)[C@H](C)NC6=O)C(=O)N[C@H]([C@@H](C)O)C(=O)N6CCC[C@@H]6C(=O)N5C4)nn3)[C@@H](O)[C@H](O)[C@@H]2O)cc1]

### Step 1: Preparation of 11-2

To a mixture of 11-1 1-azido-3,6,9, 12, 15, 18,21,24-octaoxaheptacosan-27-oic acid (370 mg, 0.79 mmol, 1.0 eq) and tetrafluorophenol (197 mg, 1.19 mmol, 1.5 eq) in DMF (5.0 mL) was added EDCI (227 mg, 1.19 mmol, 1.5 eq) and HOBt (161 mg, 1.19 mmol, 1.5 eq) at 20° C. The reaction was stirred at 20° C. for 1 hour. The mixture was purified by prep-HPLC (TFA condition) directly to afford 11-2 (445 mg, 91.5% yield) as colorless oil. Chemical Formula: C₂₅H₃₇F₄N₃O₁₀. LCMS found: [M+H]⁺=616.3 [M+2H]=308.1.

### Step 2: Preparation of 11-3

To a mixture of 11-2 (445 mg, 0.73 mmol, 1.0 eq) and Peptide 1 (2.51 g, 1.46 mmol, 2.0 eq) in DMF (20 mL) was added DIEA (118 mg, 1.46 mmol, 2.0 eq) at 20° C. The reaction was stirred at 20° C. for 1 hour. The mixture was purified by prep-HPLC (TFA condition) directly to afford 11-3 (640 mg, 40.4% yield) as a white solid. Chemical Formula: C₉₈H₁₄₃N₂₃O₂₉S2. LCMS found: [M+2H]²⁺=1086.4, [M+3H]³⁺=715.5.

### Step 3: Preparation of Compound 11

To a mixture of 11-3 (20.0 mg, 9.2 μmol, 1.0 eq) and A77 (3.6 mg, 9.2 μmol, 1.0 eq) in DMF (1.0 mL) was added a freshly prepared solution of CuSO₄ (0.4 M, 23.0 μL, 9.2 μmol, 1.0 eq) in H₂O, sodium ascorbate (7.3 mg, 36.8 μmol, 4.0 eq), and THPTA (4.1 mg, 9.2 μmol, 1.0 eq) under N₂ at 0° C. The mixture was warmed up to 20° C. and stirred at 20° C. for 2 hours. LCMS revealed one major peak with desired mass. The crude was purified by prep-HPLC (TFA condition) directly to afford Compound 11 (13.6 mg, 56.4% yield 97.7% purity) as a white solid. Chemical Formula: C₁₁₅H₁₆₆N₂₃O₃₇PS₂. Molecular Weight: 2557.78. LCMS found: [M+2H]²⁺=1279.9, [M+3H]³⁺=853.4.

### Preparation of Compound ((6R,9R,12S,15R,18R,21S,27R,30S,33S,36S,39R,42S,44aS,46S,49aR)-30-((1H-imidazol-5-yl)methyl)-12,33-bis((1H-indol-3-yl)methyl)-42-(carboxymethyl)-6-((R)-1-hydroxyethyl)-18,27-diisobutyl-15-isopropyl-36-methyl-5,8,11,14,17,20,23,26,29,32,35,38,41,44,49-12:3-pentadecaoxo-46-(1-(4-(4-(3-(7-(((2R,3S,4S,5S,6R)-3,4,5-trihydroxy-6-(2-phosphonoethyl)tetrahydro-2H-pyran-2-yl)oxy)-9H-fluoren-2-yl) ureido)butyl)-1H-1,2,3-triazol-1-yl)-3,6,9,12,15,18,21,24-octaoxaheptacosanamido)octatetracontahydro-1H-9,39-(methanodithiomethano)dipyrrolo[1,2-a:1′,2′-d][1,4,7,10,13,16,19,22,25,28,31,34,37,40,43]pentadecaazacyclopentatetracontin-21-yl)propanoic acid

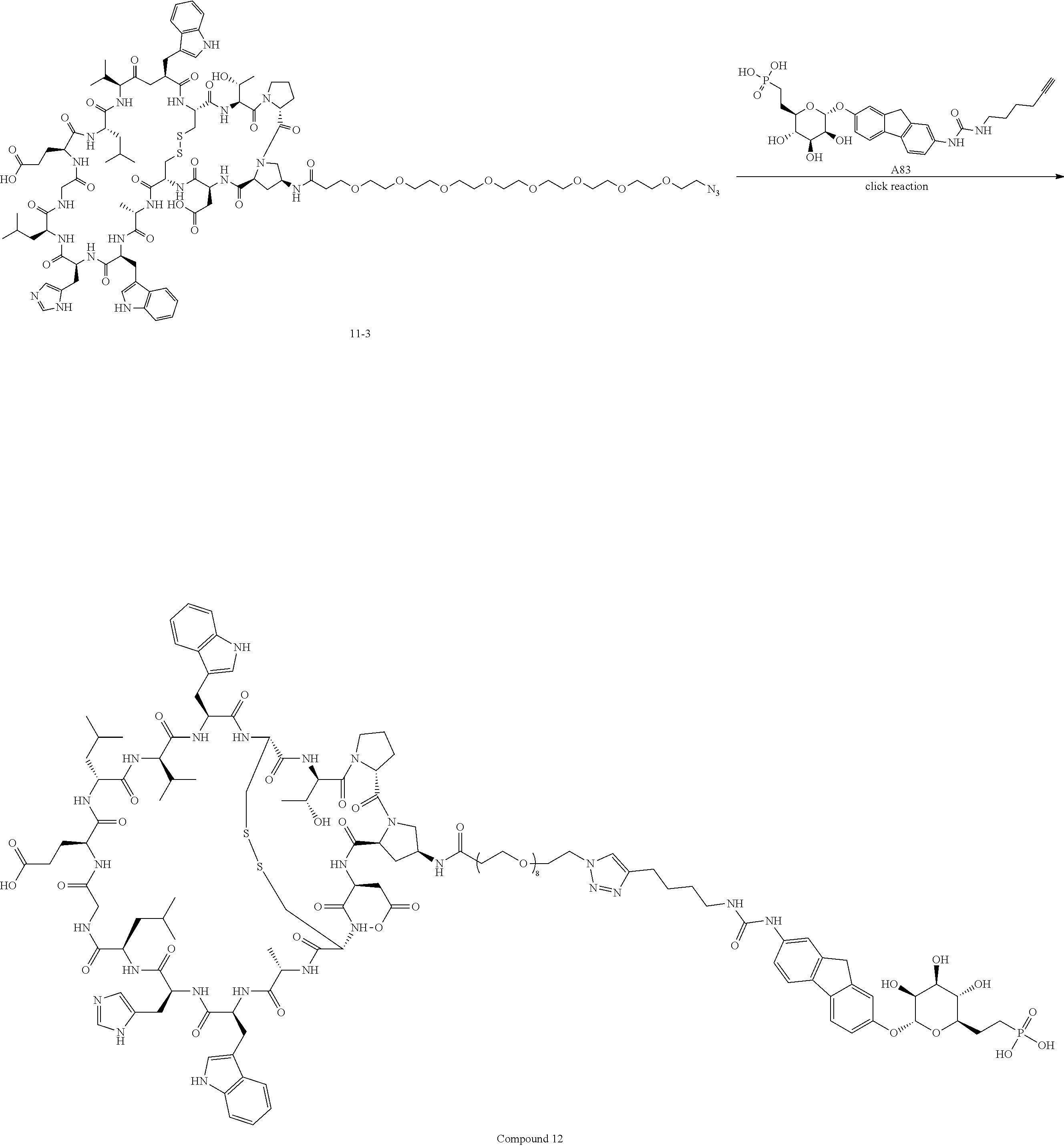

To a mixture of 11-3 (25.2 mg, 11.6 μmol, 1.0 eq) and A83 (6.5 mg, 11.6 μmol, 1.0 eq) in DMSO (0.2 mL) was added Cu(MeCN)₄PF₆ (8.6 mg, 23.2 μmol, 2.0 eq) and DIEA (8.1 μL, 46.4 μmol, 4.0 eq) under N₂ at 0° C. The mixture was warmed up to 20° C. and stirred at 20° C. for 2 hours. LCMS revealed one peak with desired mass. The crude was purified by prep-HPLC (TFA condition) directly to afford Compound 12 (1.5 mg, 4.8% yield 97.3% purity) as a white solid. Chemical Formula: Cl₂₅H₁₇₆N₂₅₀₃₈PS₂. Molecular Weight: 2731.98. LCMS found: [M+2H]²⁺=1367.0, [M+3H]³⁺=911.7

All publications and patent applications cited in this specification are herein incorporated by reference as if each individual publication or patent application were specifically and individually indicated to be incorporated by reference.

Although the foregoing invention has been described in some detail by way of illustration and example for the purpose of clarity of understanding, it will be readily apparent to one of ordinary skill in the art in light of the teaching of this invention that certain changes or modifications may be made thereto without departing from the spirit or scope of the invention as defined in the appended claims. Additionally, those skilled in the art will recognize, or be able to ascertain using no more than routine experimentation, many equivalents to the specific embodiments and methods described herein. Such equivalents are intended to be encompassed by the scope of the present application.

We claim:

1. A compound of Formula 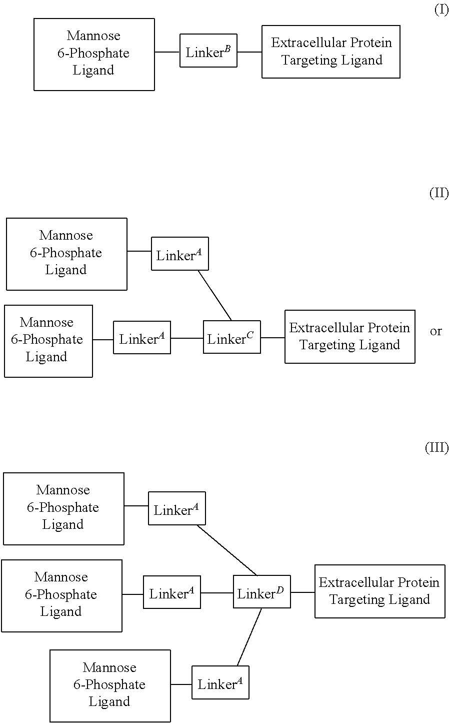
or a pharmaceutically acceptable salt thereof;
wherein:
Mannose 6-Phosphate Ligand is selected from: 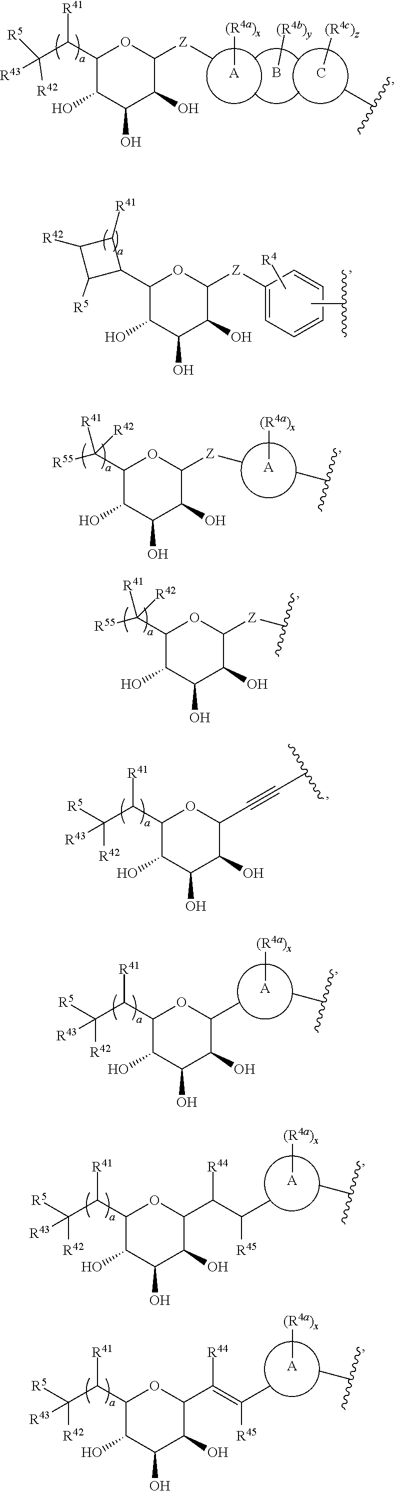 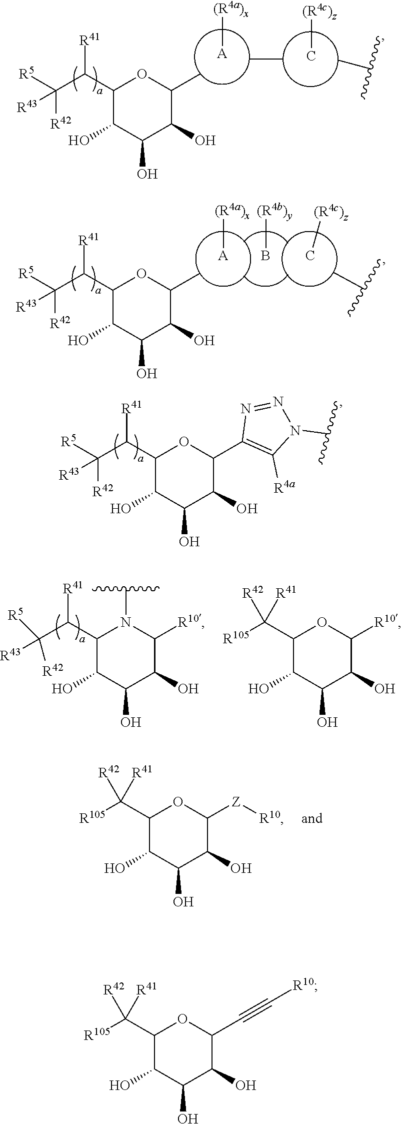
a, x, y, and z are independently 0, 1, 2, or 3;
Z is NR⁸, O, S, NC(O)R³, or CR⁴R⁸;
\textcircled{A} is cycloalkyl, heterocycle, aryl or heteroaryl;
\textcircled{B} is a nonaromatic cycloalkyl or heterocycle;
\textcircled{C} is aryl or heteroaryl;
R³ at each occurrence is independently selected from hydrogen, alkyl, haloalkyl, arylalkyl, heteroarylalkyl, alkenyl, alkynyl, aryl, heteroaryl, heterocycle, —OR⁸, and —NR⁸R⁹;
R⁴, R^{4a}, R^{4b}, and R^{4c} are independently selected at each occurrence from hydrogen, F, Cl, Br, I, alkyl, haloalkyl, arylalkyl, heteroarylalkyl, alkenyl, alkynyl, aryl, heteroaryl, heterocycle, cyano, —OR⁶, —NR⁶R⁷, C(O)R³, S(O)R³, C(S)R³, and S(O)₂R³;
R⁴¹, R⁴², R⁴³, R⁴⁴, and R⁴⁵ are independently selected at each occurrence from hydrogen, alkyl, haloalkyl, F, arylalkyl, heteroarylalkyl, alkenyl, alkynyl, aryl, heteroaryl, and heterocycle;
R⁵⁰ is hydrogen, alkyl, haloalkyl, arylalkyl, heteroarylalkyl, alkenyl, alkynyl, aryl, heteroaryl, heterocycle, —OR⁶, or —NR⁶R⁷;
R⁵ is selected from 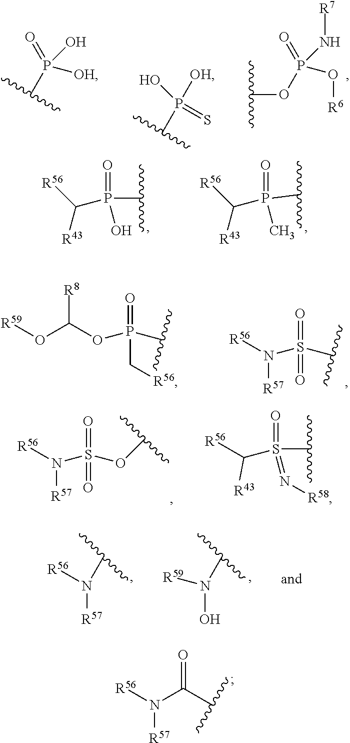
R⁵⁵ is selected from 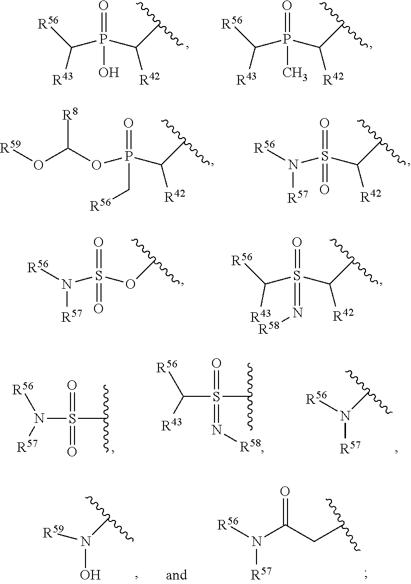
R⁵⁶ is independently selected from hydrogen, alkyl, alkenyl, alkynyl, haloalkyl, —O-alkyl, hydroxyl, cyano, heterocyclyl, aryl, and heteroaryl wherein each group except for hydrogen, hydroxyl, and cyano may optionally be substituted with 1, 2, or 3 independently selected R^{9a} substituents as allowed by valence;
R⁵⁷ is selected from hydrogen, alkyl, alkenyl, alkynyl, haloalkyl, —O-alkyl, aryl, heteroaryl, C(O)H, C(O)alkyl, C(O)alkenyl, C(O)alkynyl, C(O)haloalkyl, C(O) aryl, C(O)heteroaryl, C(O)O-alkyl, C(O)O-alkenyl, C(O)O-alkynyl, C(O)O-haloalkyl, C(O)O-aryl, C(O)O-heteroaryl and 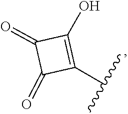 [SMILES: *c1c(O)c(=O)c1=O]
wherein each group except for hydrogen or C(O)H may optionally be substituted with 1, 2, or 3 independently selected R^{9b} substituents as allowed by valence;
or R⁵⁶, R⁵⁷, and the nitrogen to which they are attached form a 5 to 7 membered heterocyclic ring which can be optionally substituted by alkyl, heterocycle, aryl, and heteroaryl;
R⁵⁸ is selected from hydrogen, alkyl, C(O)alkyl, C(O)O-alkyl, C(O) aryl, C(O)heteroaryl, C(O)O-aryl, C(O)O-arylalkyl, and C(O)O-heteroaryl, wherein each group may optionally be substituted with 1, 2, or 3 independently selected R⁹° C. substituents as allowed by valence;
R⁵⁹ is selected from C(O)alkyl, C(O)O-alkyl, C(O) aryl, C(O)heteroaryl, C(O)O-aryl, and C(O)O-heteroaryl, wherein each group may optionally be substituted with 1, 2, or 3 independently selected R^{9d} substituents as allowed by valence;
R⁶ and R⁷ are independently selected at each occurrence from hydrogen, alkyl, arylalkyl, heteroarylalkyl, alkenyl, alkynyl, aryl, haloalkyl, heteroaryl, heterocycle,-alkyl-OR⁸, -alkyl-NR⁸R⁹, C(O)R³, S(O)R³, C(S)R³, and S(O)₂R³;
R⁸ is independently selected at each occurrence from hydrogen, alkyl, arylalkyl, heteroarylalkyl, alkenyl, alkynyl, aryl, heteroaryl, and heterocycle;
R⁹ is independently selected at each occurrence from hydrogen, alkyl, arylalkyl, heteroarylalkyl, alkenyl, alkynyl, aryl, heteroaryl, and heterocycle;
R^{9a}, R^{9b}b, R^{9c}, and R^{9d} are independently selected at each occurrence from hydrogen, F, Cl, Br, I, alkyl, haloalkyl, arylalkyl, heteroarylalkyl, alkenyl, alkynyl, aryl, heteroaryl, heterocycle, cyano, —OR⁶, —NR⁶R⁷, C(O)R³, S(O)R³, C(S)R³, and S(O)₂R³;
R¹⁰ is selected from hydrogen, alkyl, aryl, heteroaryl, arylalkyl, heteroarylalkyl, alkyl-O-alkyl, C(O) aryl, C(O)alkyl, C(O) arylalkyl, C(O)heteroarylalkyl, each of which R¹⁰ except hydrogen is optionally substituted with 1, 2, 3, or 4 substituents selected from alkyl, alkenyl, alkynyl, haloalkyl, —OR⁶, F, Cl, Br, I, —NR⁶R⁷, heterocycle, heteroaryl, aryl, cyano, nitro, hydroxyl, amide, —SR³, —S(O)(NR⁶)R³, —NRC(O)R³, —C(O)NR⁶R⁷, —C(O)OR³, and —C(O)R³;
or R¹⁰ is 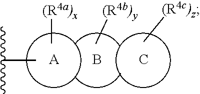
R¹⁰′ is selected from hydrogen, alkyl, heteroaryl, arylalkyl, heteroarylalkyl, each of which R¹⁰′ except hydrogen is optionally substituted with 1, 2, 3, or 4 substituents selected from alkyl, alkenyl, alkynyl, haloalkyl, —OR⁶, F, Cl, Br, I, —NR⁶R⁷, heterocycle, heteroaryl, aryl, cyano, nitro, hydroxyl, amide, —SR³, —S(O)(NR⁶)R³, —NR⁸° C. (O)R³, —C(O)NR⁶R⁷, —C(O)OR³, and —C(O)R³;
R¹⁰⁵ is selected from 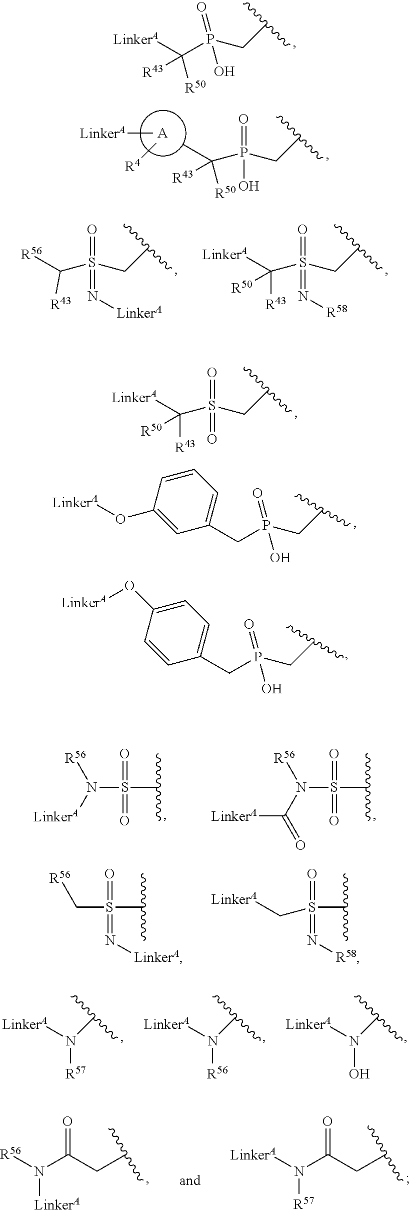
Linker^{A} and Linker^{B} are independently selected from: 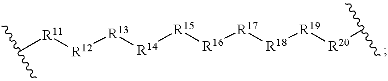
wherein:
R¹¹, R¹², R¹³, R¹⁴, R¹⁵, R¹⁶, R¹⁷, R¹⁸, R¹⁹, and R²⁰ are independently at each occurrence selected from the group consisting of a bond, alkyl, —C(O)—, —C(O)O—, —OC(O)—, —SO₂—, —S(O)—, —C(S)—, —C(O)NR⁶—, —NR⁹C(O)—, —C(O)N(OH)—, —N(OH)C(O)—, —O—, —S—, —NR⁶—, —C(R²¹R²¹)—, —S(O)(═N—R⁴⁴)—, —S(O)(═NR), —P(O)(R³)O—, —P(O)(R³)—, a divalent residue of a natural or unnatural amino acid, alkenyl, alkynyl, haloalkyl, alkoxy, aryl, heterocycle, heteroaryl, —CH₂CH₂—[O—(CH₂)₂]ₙ—O—, —CH₂CH₂—[O—(CH₂)₂], —NR⁶—, —CH₂CH₂—[O—(CH₂)₂]ₙ—, -[—(CH₂)₂—O-]ₙ-,
[O—(CH₂)₂]ₙ—, —[O—CH(CH₃)C(O)]ₙ-, —[C(O)—CH(CH₃)—O]ₙ, —[O—CH₂C(O)]ₙ—, —[C(O)—CH₂—O]ₙ, a divalent residue of a fatty acid, a divalent residue of an unsaturated or saturated mono- or di-carboxylic acid; each of which is optionally substituted with 1, 2, 3, or 4 substituents independently selected from R²¹;
Linker^{C} is selected from: 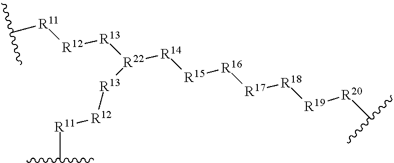
wherein:
R²² is independently at each occurrence selected from the group consisting of alkyl, —C(O)N—, —NC(O)—, —N—, —C(R²¹)—, —P(O)O—, —P(O)—, —P(O)(NR⁶R⁷)N—, alkenyl, haloalkyl, aryl, heterocycle, and heteroaryl, each of which is optionally substituted with 1, 2, 3, or 4 substituents independently selected from R²¹;
Linker^{D} is selected from: 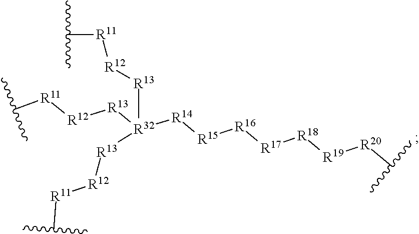
wherein:
R³² is independently at each occurrence selected from the group consisting of alkyl, N⁺ X⁻, —C—, alkenyl, haloalkyl, aryl, heterocycle, and heteroaryl, each of which except N′X; and —C— is optionally substituted with 1, 2, 3, or 4 substituents independently selected from R²¹;
X⁻ is an anionic group, for example Br⁻ or Cl⁻;
n is independently selected at each instance from 0, 1, 2, 3, 4, 5, 6, 7, 8, 9, or 10;
R²¹ is independently at each occurrence selected from the group consisting of hydrogen, alkyl, alkenyl, alkynyl, F, Cl, Br, I, hydroxyl, alkoxy, azide, amino, cyano, —NR⁶R⁷, —NR⁸SO₂R³, —NR⁸S(O)R³, haloalkyl, aryl, heteroaryl, and heterocycle; and
Extracellular Protein Targeting Ligand is a means for binding a targeted disease-mediating extracellular protein.

2. The compound of claim 1, wherein the compound is of Formula: 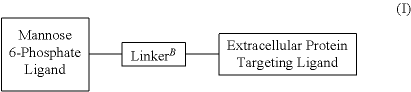
or a pharmaceutically acceptable salt thereof.

3. The compound of claim 1, wherein the compound is of Formula: 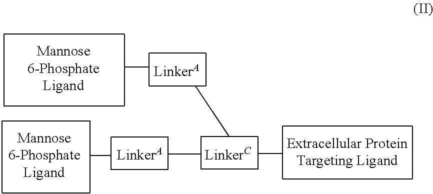
or a pharmaceutically acceptable salt thereof.

4. The compound of claim 1, wherein the compound is of Formula: 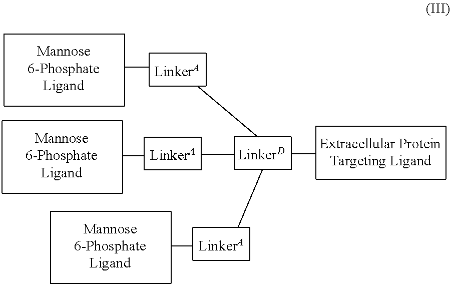
or a pharmaceutically acceptable salt thereof.

5. The compound of claim 2, wherein Mannose 6-Phosphate Ligand is selected from:

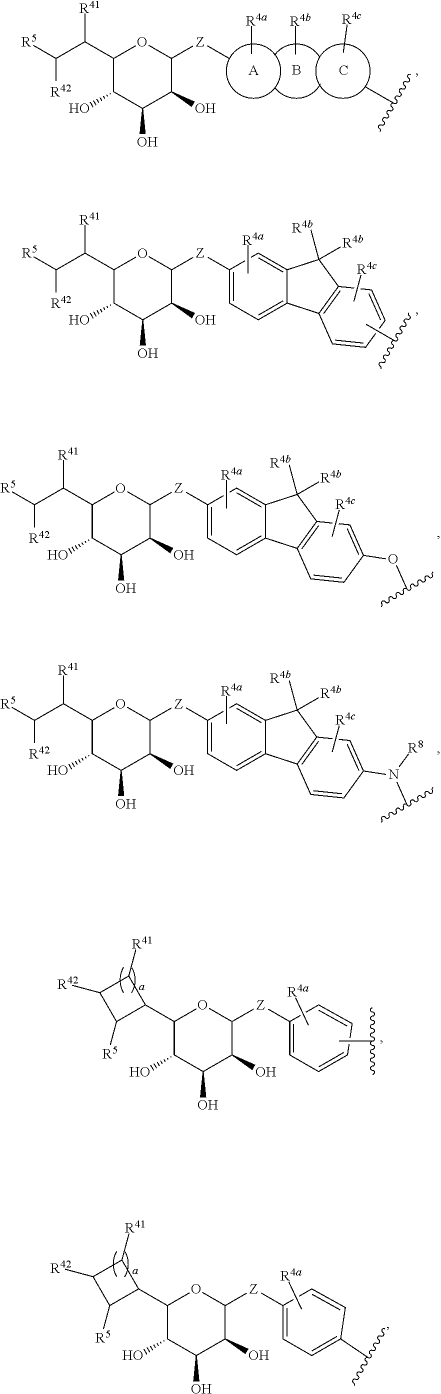

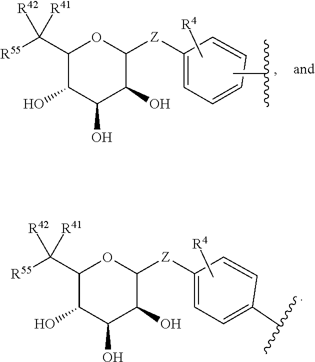

6. The compound of claim 2, wherein the Mannose 6-Phosphate Ligand is selected from:

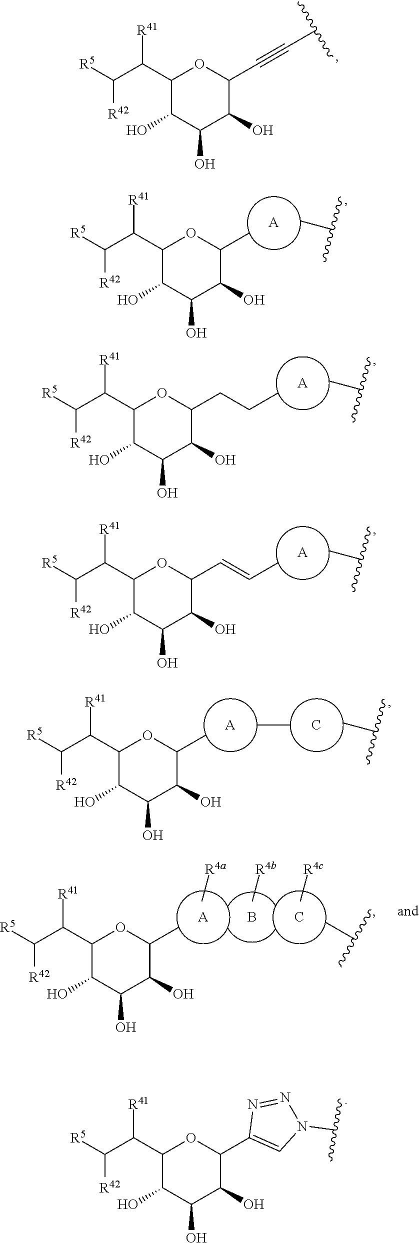

7. The compound of claim 2, wherein Mannose 6-Phosphate Ligand is

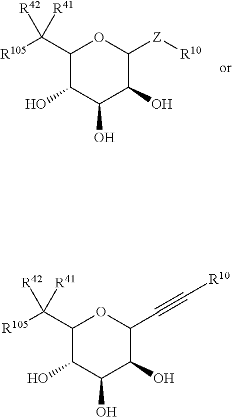

8. The compound of claim 2, wherein Mannose 6-Phosphate Ligand is

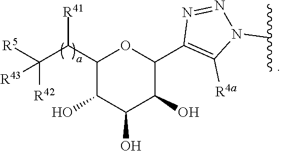

9. The compound of claim 2, wherein Mannose 6-Phosphate Ligand is

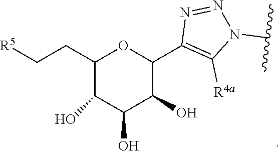

10. The compound of claim 2, wherein Mannose 6-Phosphate Ligand is

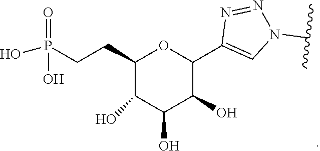 [SMILES: *n1cc(C2O[C@H](CCP(=O)(O)O)[C@@H](O)[C@H](O)[C@@H]2O)nn1]

11. The compound of claim 1, wherein R⁴, R^{4a}, R^{4b}, and R^{4c} are hydrogen.

12. The compound of claim 11, wherein R⁵ is selected from:

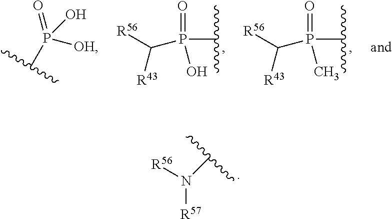

13. The compound of claim 11, wherein R⁵⁵ is selected from:

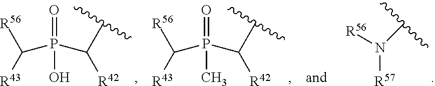

14. The compound of claim 1, wherein Linker^{B} is: 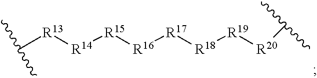
wherein:
R¹³, R¹⁴, R¹⁵, R¹⁶, R¹⁷, R¹⁸, R¹⁹, and R²⁰ are independently at each occurrence selected from the group consisting of a bond, alkyl, —C(O)—, —C(O)O—, —OC(O)—, —SO₂—, —S(O)—, —C(S)—, —C(O)NR⁶—, —NR⁶C(O)—, —O—, —S—, —NR⁶, —P(O)(R³)O—, —P(O)(R³)—, alkenyl, alkynyl, haloalkyl, alkoxy, aryl, heterocycle, heteroaryl, —CH₂CH₂—[O—(CH₂)₂]ₙ—O—, —CH₂CH₂—[O—(CH₂)₂]ₙ—NR⁶—, —CH₂CH₂—[O—(CH₂)₂]ₙ—, -[—(CH₂)₂—O—]n—, —[O—(CH₂)₂]ₙ—, —[O—CH(CH₃)C(O)]ₙ—, —[C(O)—CH(CH₃)—O]ₙ—, —[O—CH₂C(O)]ₙ—, —[C(O)—CH₂—O]ₙ—, or an amino acid; each of which is optionally substituted with 1 substituent independently selected from R²¹.

15. The compound of claim 14, wherein R²¹ is independently at each occurrence selected from the group consisting of hydrogen, alkyl, alkenyl, alkynyl, F, Cl, Br, I, hydroxyl, alkoxy, azide, amino, cyano, —NR⁶R⁷, —NR⁸SO₂R³, —NR⁸S(O)R³, haloalkyl, aryl, heteroaryl, and heterocycle.

16. The compound of claim 1, wherein the Extracellular Protein Targeting Ligand targets an immunoglobin.

17. The compound of claim 2, wherein the Extracellular Protein Targeting Ligand targets an immunoglobin.

18. The compound of claim 1, wherein the Extracellular Protein Targeting Ligand is:

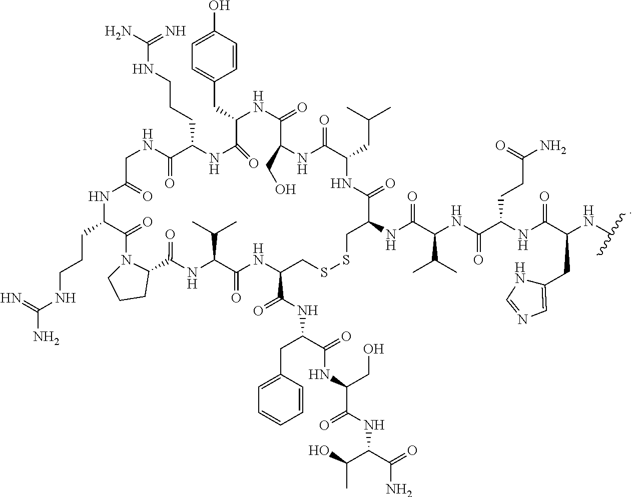 [SMILES: *N[C@@H](Cc1cnc[nH]1)C(=O)N[C@@H](CCC(N)=O)C(=O)N[C@H](C(=O)N[C@H]1CSSC[C@@H](C(=O)N[C@@H](Cc2ccccc2)C(=O)N[C@@H](CO)C(=O)N[C@H](C(N)=O)[C@@H](C)O)NC(=O)[C@H](C(C)C)NC(=O)[C@@H]2CCCN2C(=O)[C@H](CCCNC(=N)N)NC(=O)CNC(=O)[C@H](CCCNC(=N)N)NC(=O)[C@H](Cc2ccc(O)cc2)NC(=O)[C@H](CO)NC(=O)[C@H](CC(C)C)NC1=O)C(C)C]

19. A compound selected from: 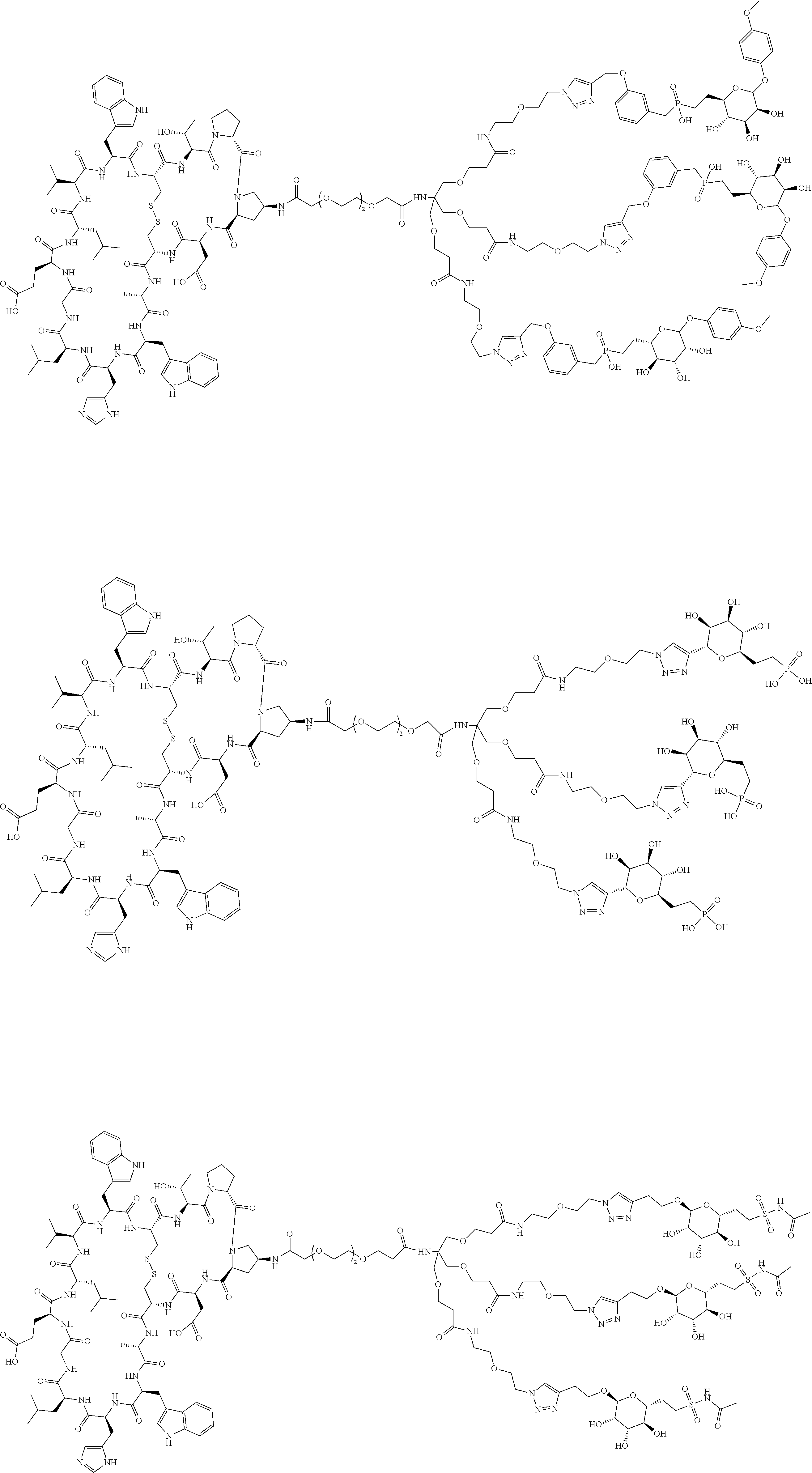 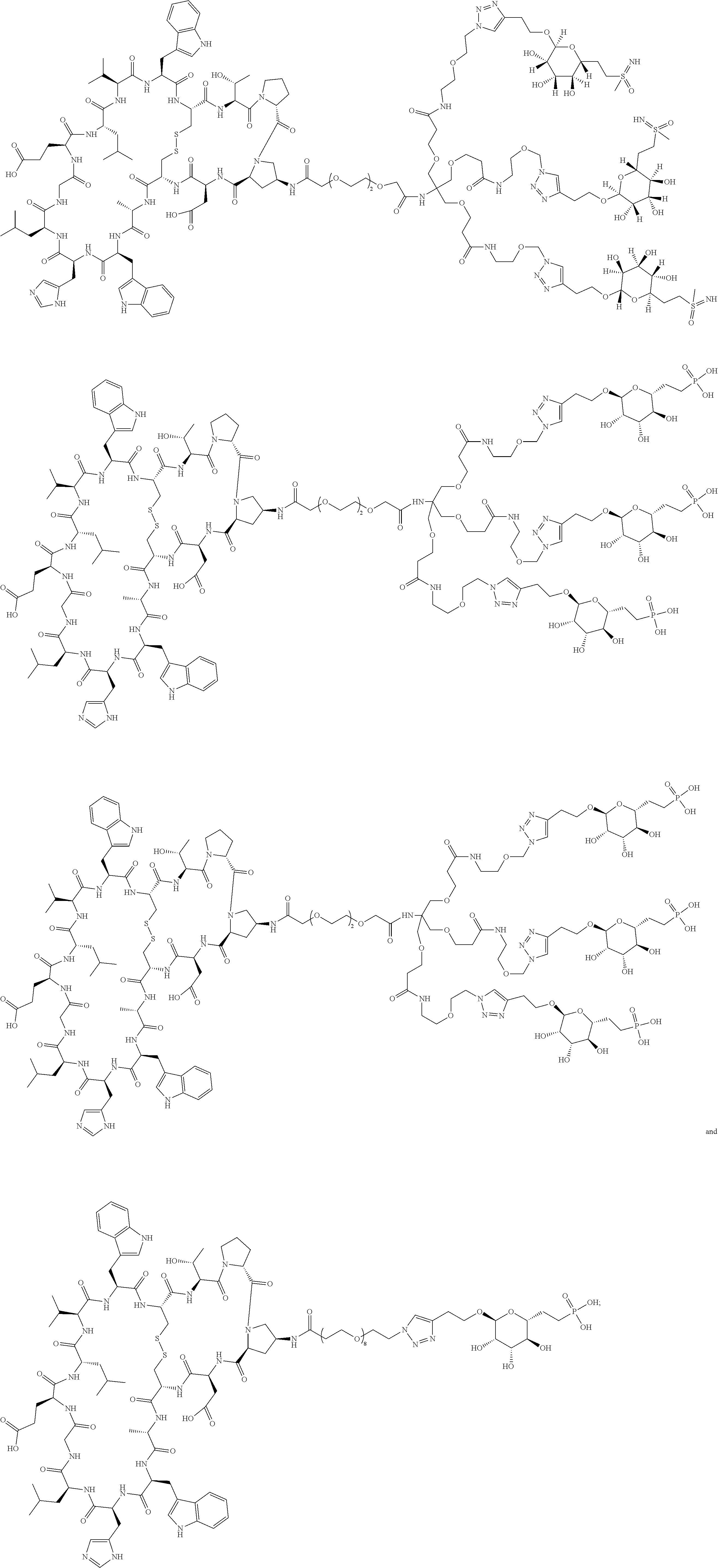
or a pharmaceutically acceptable salt thereof.

20. A compound selected from: 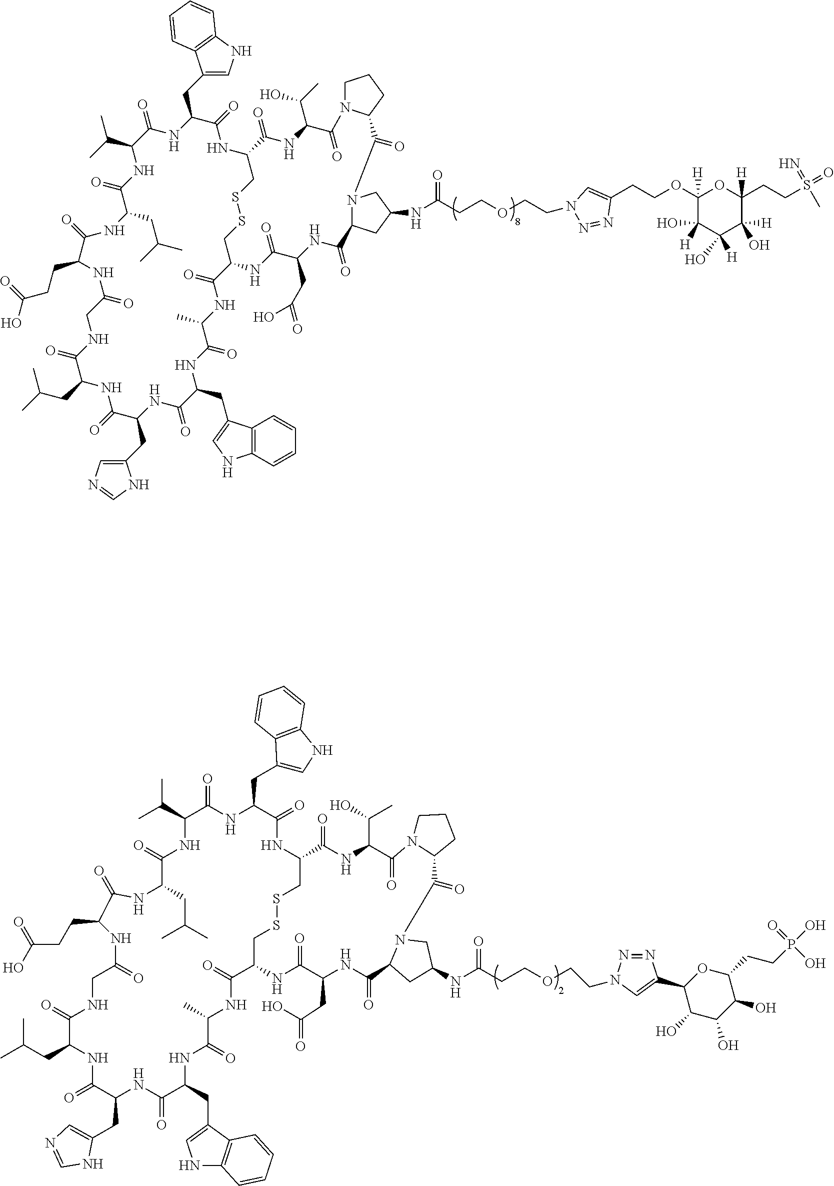 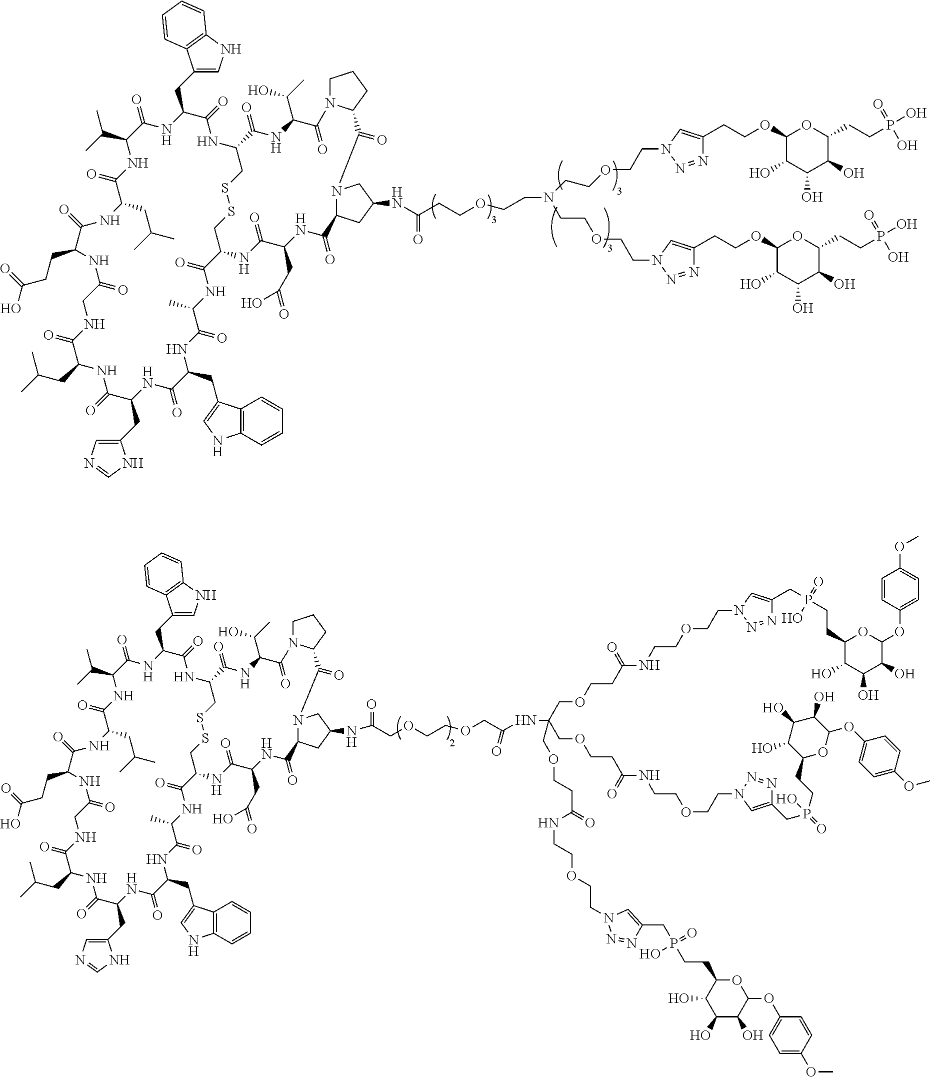 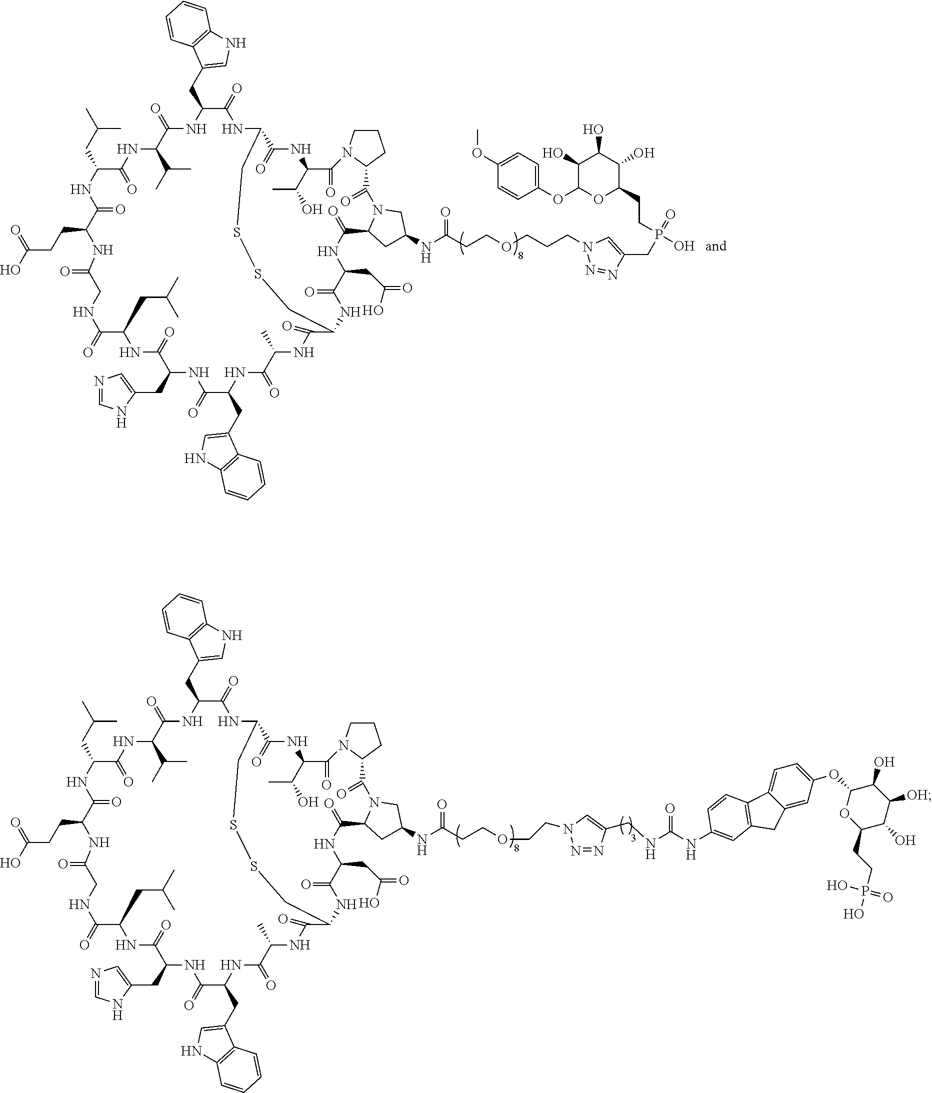
or a pharmaceutically acceptable salt thereof.


SEQUENCE LISTING (504 sequences)

SEQ ID NO: 1 - protein, 6 aa, synthetic construct
MLKKIE

SEQ ID NO: 2 - protein, 19 aa, synthetic construct
VEPNCDIHVMWEWECFERL

SEQ ID NO: 3 - protein, 13 aa, synthetic construct
TVFTSWEEYLDWV

SEQ ID NO: 4 - protein, 13 aa, synthetic construct
  MOD_RES 7: note = N-Methylation
  MOD_RES 9: note = N-Methylation
  CROSSLNK 1..13: note = N-Terminal Acetyl to C-terminal Cysteine
  SITE 1: note = N-Terminal Acetyl
  SITE 6: note = L-4-phenyl-phenylalanine
  SITE 8: note = L-4-phenyl-phenylalanine
FVPTTXAXAEAPC

SEQ ID NO: 5 - protein, 13 aa, synthetic construct
  MOD_RES 7: note = N-Methylation
  MOD_RES 9: note = N-Methylation
  SITE 8: note = L-4-phenyl-phenylalanine
  CROSSLNK 1..13: note = N-Terminal Acetyl to C-Terminal Cysteine
  SITE 1: note = N-Terminal Acetylation
FVDTTSFXENSPC

SEQ ID NO: 6 - protein, 13 aa, synthetic construct
  SITE 1: note = N-Terminal Acetyl
  SITE 6: note = L-4-phenyl-phenylalanine
  MOD_RES 7: note = N-Methylation
  SITE 8: note = L-4-phenyl-phenylalanine
  MOD_RES 9: note = N-Methylation
  CROSSLNK 1..13: note = N-Terminal Acetyl to C-terminal Cysteine
FVSTTXAXADRPC

SEQ ID NO: 7 - protein, 13 aa, synthetic construct
  SITE 1: note = N-Terminal Acetyl
  MOD_RES 7: note = N-Methylation
  SITE 8: note = L-4-phenyl-phenylalanine
  MOD_RES 9: note = N-Methylation
  CROSSLNK 1..13: note = N-Terminal Acetyl to C-terminal Cysteine
FVDTTSFXANSPC

SEQ ID NO: 8 - protein, 13 aa, synthetic construct
  MOD_RES 7: note = N-Methylation
  SITE 8: note = L-4-phenyl-phenylalanine
  MOD_RES 9: note = N-Methylation
  SITE 1: note = N-Terminal Acetyl
  CROSSLNK 1..13: note = N-Terminal Acetyl to C-terminal Cysteine
FVDSTTAXANHPC

SEQ ID NO: 9 - protein, 13 aa, synthetic construct
  SITE 1: note = N-Terminal Acetyl
  MOD_RES 7: note = N-Methylation
  MOD_RES 9: note = N-Methylation
  SITE 8: note = L-4-phenyl-phenylalanine
  CROSSLNK 1..13: note = N-Terminal Acetyl to C-Terminal Cysteine
FVDTTSFXAESPC

SEQ ID NO: 10 - protein, 13 aa, synthetic construct
  SITE 1: note = N-Terminal Acetyl
  CROSSLNK 1..13: note = N-Terminal Acetyl to C-Terminal Cysteine
  MOD_RES 7: note = N-Methylation
  MOD_RES 8: note = 4-(Trifluoromethyl)-L-phenylalanine
  MOD_RES 9: note = N-Methylation
FVDTTSFFAESPC

SEQ ID NO: 11 - protein, 13 aa, synthetic construct
  SITE 1: note = N-Terminal Acetyl
  MOD_RES 7: note = N-Methylation
  SITE 8: note = L-4-phenyl-phenylalanine
  MOD_RES 9: note = N-Methylation
  CROSSLNK 1..13: note = N-Terminal Acetyl to C-Terminal Cysteine
FVDTTSAXAKSPC

SEQ ID NO: 12 - protein, 13 aa, synthetic construct
  SITE 1: note = N-Terminal Acetyl
  MOD_RES 7: note = N-Methylation
  SITE 6: note = L-4-phenyl-phenylalanine
  SITE 8: note = L-4-phenyl-phenylalanine
  MOD_RES 9: note = L-4-phenyl-phenylalanine
  CROSSLNK 1..13: note = N-Terminal Acetyl to C-Terminal Cysteine
FVSTTXAXADSPC

SEQ ID NO: 13 - protein, 13 aa, synthetic construct
  MOD_RES 7: note = N-Methylation
  SITE 1: note = N-Terminal Acetyl
  SITE 8: note = L-4-phenyl-phenylalanine
  MOD_RES 9: note = N-Methylation
  CROSSLNK 1..13: note = N-Terminal Acetyl to C-Terminal Cysteine
FVSTTSFXADRPC

SEQ ID NO: 14 - protein, 13 aa, synthetic construct
  SITE 1: note = N-Terminal Acetyl
  MOD_RES 7: note = N-Methylation
  SITE 8: note = L-4-phenyl-phenylalanine
  MOD_RES 9: note = N-Methylation
  CROSSLNK 1..13: note = N-Terminal Acetyl to C-Terminal Cysteine
FVDTTXAXAESPC

SEQ ID NO: 15 - protein, 13 aa, synthetic construct
  SITE 1: note = N-Terminal Acetyl
  MOD_RES 6: note = 4-(Trifluoromethyl)-L-phenylalanine
  MOD_RES 7: note = N-Methylation
  MOD_RES 8: note = 4-(Trifluoromethyl)-L-phenylalanine
  MOD_RES 9: note = N-Methylation
  CROSSLNK 1..13: note = N-Terminal Acetyl to C-Terminal Cysteine
FVSTTFAFAERPC

SEQ ID NO: 16 - protein, 13 aa, synthetic construct
  SITE 1: note = N-Terminal Acetyl
  MOD_RES 7: note = N-Methylation
  SITE 8: note = L-4-phenyl-phenylalanine
  MOD_RES 9: note = N-Methylation
  CROSSLNK 1..13: note = N-Terminal Acetyl to C-Terminal Cysteine
FVSTTSFXAESPC

SEQ ID NO: 17 - protein, 13 aa, synthetic construct
  SITE 1: note = N-Terminal Acetyl
  SITE 6: note = L-4-phenyl-phenylalanine
  MOD_RES 7: note = N-Methylation
  SITE 8: note = L-4-phenyl-phenylalanine
  MOD_RES 9: note = N-Methylation
  CROSSLNK 1..13: note = N-Terminal Acetyl to C-Terminal Cysteine
FVSTTXFXAESPC

SEQ ID NO: 18 - protein, 13 aa, synthetic construct
  SITE 1: note = N-Terminal Acetylation
  SITE 6: note = L-4-phenyl-phenylalanine
  MOD_RES 7: note = N-Methylation
  MOD_RES 8: note = 3,4-Dichloro-L-phenylalanine
  MOD_RES 9: note = N-Methylation
  CROSSLNK 1..13: note = N-Terminal Acetyl to C-Terminal Cysteine
FVSTTXAFADRPC

SEQ ID NO: 19 - protein, 13 aa, synthetic construct
  SITE 1: note = N-Terminal Acetyl
  SITE 6: note = L-4-phenyl-phenylalanine
  MOD_RES 7: note = N-Methylation
  MOD_RES 8: note = 3-Fluoro-L-phenylalanine
  MOD_RES 9: note = N-Methylation
  CROSSLNK 1..13: note = N-Terminal Acetyl to C-Terminal Cysteine
FVSTTXAFADRPC

SEQ ID NO: 20 - protein, 13 aa, synthetic construct
  SITE 1: note = N-Terminal Acetyl
  MOD_RES 7: note = N-Methylation
  SITE 8: note = L-4-phenyl-phenylalanine
  MOD_RES 9: note = N-Methylation
  CROSSLNK 1..13: note = N-Terminal Acetyl to C-Terminal Cysteine
FVDTTAFXAEAPC

SEQ ID NO: 21 - protein, 13 aa, synthetic construct
  SITE 1: note = N-Terminal Acetyl
  MOD_RES 7: note = N-Methylation
  SITE 8: note = L-4-phenyl-phenylalanine
  MOD_RES 9: note = L-4-phenyl-phenylalanine
  CROSSLNK 1..13: note = N-Terminal Acetyl to C-Terminal Cysteine
FVDTTFAXAESPC

SEQ ID NO: 22 - protein, 13 aa, synthetic construct
  SITE 1: note = N-Terminal Acetyl
  MOD_RES 7: note = N-Methylation
  SITE 8: note = L-4-phenyl-phenylalanine
  MOD_RES 9: note = N-Methylation
  CROSSLNK 1..13: note = N-Terminal Acetyl to C-Terminal Cysteine
FVDTTAFXAQAPC

SEQ ID NO: 23 - protein, 13 aa, synthetic construct
  SITE 1: note = N-Terminal Acetyl
  SITE 6: note = L-4-phenyl-phenylalanine
  MOD_RES 7: note = N-Methylation
  SITE 8: note = L-4-phenyl-phenylalanine
  MOD_RES 9: note = N-Methylation
  CROSSLNK 1..13: note = N-Terminal Acetyl to C-Terminal Cysteine
FVPTTXAXADRPC

SEQ ID NO: 24 - protein, 13 aa, synthetic construct
  SITE 1: note = N-Terminal Acetyl
  SITE 6: note = L-4-phenyl-phenylalanine
  MOD_RES 7: note = N-Methylation
  SITE 8: note = L-4-phenyl-phenylalanine
  MOD_RES 9: note = N-Methylation
  CROSSLNK 1..13: note = N-Terminal Acetyl to C-Terminal Cysteine
FVSTTXAXAPSPC

SEQ ID NO: 25 - protein, 13 aa, synthetic construct
  SITE 1: note = N-Terminal Acetyl
  SITE 8: note = L-4-phenyl-phenylalanine
  MOD_RES 7: note = N-Methylation
  MOD_RES 9: note = N-Methylation
  CROSSLNK 1..13: note = N-Terminal Acetyl to C-Terminal Cysteine
FVPTTAFXAEAPC

SEQ ID NO: 26 - protein, 13 aa, synthetic construct
  SITE 1: note = N-Terminal Acetyl
  MOD_RES 7: note = N-Methylation
  SITE 8: note = L-4-phenyl-phenylalanine
  MOD_RES 9: note = N-Methylation
  CROSSLNK 1..13: note = N-Terminal Acetyl to C-Terminal Cysteine
FVATTFAXAKAPC

SEQ ID NO: 27 - protein, 13 aa, synthetic construct
  SITE 1: note = N-Terminal Acetyl
  MOD_RES 7: note = N-Methylation
  SITE 8: note = L-4-phenyl-phenylalanine
  MOD_RES 9: note = N-Methylation
  CROSSLNK 1..13: note = N-Terminal Acetyl to C-Terminal Cysteine
FVNTTFAXAKAPC

SEQ ID NO: 28 - protein, 13 aa, synthetic construct
  SITE 1: note = N-Terminal Acetyl
  MOD_RES 7: note = N-Methylation
  SITE 8: note = L-4-phenyl-phenylalanine
  MOD_RES 9: note = N-Methylation
  CROSSLNK 1..13: note = N-Terminal Acetyl to C-Terminal Cysteine
FVSTTFAXAEAPC

SEQ ID NO: 29 - protein, 13 aa, synthetic construct
  SITE 1: note = N-Terminal Acetyl
  SITE 6: note = L-4-phenyl-phenylalanine
  MOD_RES 7: note = N-Methylation
  SITE 8: note = L-4-phenyl-phenylalanine
  MOD_RES 9: note = N-Methylation
  CROSSLNK 1..13: note = N-Terminal Acetyl to C-Terminal Cysteine
FVSTTXAXAESPC

SEQ ID NO: 30 - protein, 13 aa, synthetic construct
  SITE 1: note = N-Terminal Acetyl
  SITE 6: note = L-4-phenyl-phenylalanine
  MOD_RES 7: note = N-Methylation
  MOD_RES 8: note = 3,4-Dichloro-L-phenylalanine
  MOD_RES 9: note = N-Methylation
  CROSSLNK 1..13: note = N-Terminal Acetyl to C-Terminal Cysteine
FVDTTXAFAESPC

SEQ ID NO: 31 - protein, 13 aa, synthetic construct
  SITE 1: note = N-Terminal Acetyl
  MOD_RES 7: note = N-Methylation
  SITE 8: note = L-4-phenyl-phenylalanine
  MOD_RES 9: note = N-Methylation
  CROSSLNK 1..13: note = N-Terminal Acetyl to C-Terminal Cysteine
FENTTFAXAASPC

SEQ ID NO: 32 - protein, 13 aa, synthetic construct
  SITE 1: note = N-Terminal Acetyl
  MOD_RES 7: note = N-Methylation
  MOD_RES 8: note = 4-Chloro-L-phenylalanine
  MOD_RES 9: note = N-Methylation
  CROSSLNK 1..13: note = N-Terminal Acetyl to C-Terminal Cysteine
FVPTTFAFAPAPC

SEQ ID NO: 33 - protein, 13 aa, synthetic construct
  SITE 1: note = N-Terminal Acetyl
  MOD_RES 7: note = N-Methylation
  MOD_RES 8: note = 4-Chloro-L-phenylalanine
  MOD_RES 9: note = N-Methylation
  CROSSLNK 1..13: note = N-Terminal Acetyl to C-Terminal Cysteine
FVPTTFAFADAPC

SEQ ID NO: 34 - protein, 13 aa, synthetic construct
  SITE 1: note = N-Terminal Acetyl
  SITE 3: note = L-Homoserine
  MOD_RES 7: note = N-Methylation
  MOD_RES 8: note = 4-Chloro-L-phenylalanine
  MOD_RES 9: note = N-Methylation
  CROSSLNK 1..13: note = N-Terminal Acetyl to C-Terminal Cysteine
FVXTTFAFADAPC

SEQ ID NO: 35 - protein, 13 aa, synthetic construct
  SITE 1: note = N-Terminal Acetyl
  MOD_RES 7: note = N-Methylation
  MOD_RES 8: note = 4-Chloro-L-phenylalanine
  MOD_RES 9: note = N-Methylation
  CROSSLNK 1..13: note = N-Terminal Acetyl to C-Terminal Cysteine
FVATTFAFANAPC

SEQ ID NO: 36 - protein, 13 aa, synthetic construct
  SITE 1: note = N-Terminal Acetyl
  MOD_RES 7: note = N-Methylation
  MOD_RES 8: note = 4-Chloro-L-phenylalanine
  MOD_RES 9: note = N-Methylation
  CROSSLNK 1..13: note = N-Terminal Acetyl to C-Terminal Cysteine
FVPTTVAFAEAPC

SEQ ID NO: 37 - protein, 13 aa, synthetic construct
  SITE 1: note = N-Terminal Acetyl
  MOD_RES 7: note = N-Methylation
  SITE 8: note = L-4-phenyl-phenylalanine
  MOD_RES 9: note = N-Methylation
  MOD_RES 10: note = N-Methylation
  CROSSLNK 1..13: note = N-Terminal Acetyl to C-Terminal Cysteine
FVNTTFAXAEAPC

SEQ ID NO: 38 - protein, 13 aa, synthetic construct
  SITE 1: note = N-Terminal Acetyl
  MOD_RES 7: note = N-Methylation
  SITE 8: note = L-4-phenyl-phenylalanine
  MOD_RES 9: note = N-Methylation
  CROSSLNK 1..13: note = N-Terminal Acetyl to C-Terminal Cysteine
FVPTTFAXAEAPC

SEQ ID NO: 39 - protein, 13 aa, synthetic construct
  SITE 1: note = N-Terminal Acetyl
  SITE 6: note = L-4-phenyl-phenylalanine
  MOD_RES 7: note = N-Methylation
  SITE 8: note = L-4-phenyl-phenylalanine
  MOD_RES 9: note = N-Methylation
  CROSSLNK 1..13: note = N-Terminal Acetyl to C-Terminal Cysteine
FVPTTXAXAAAPC

SEQ ID NO: 40 - protein, 16 aa, synthetic construct
HMVCLAYRGRPVCFAL

SEQ ID NO: 41 - protein, 16 aa, synthetic construct
HMVCLSYRGRPVCFSL

SEQ ID NO: 42 - protein, 16 aa, synthetic construct
HQVCLSYRGRPVCFST

SEQ ID NO: 43 - protein, 16 aa, synthetic construct
QMRCLSYKGRRVCLWL

SEQ ID NO: 44 - protein, 16 aa, synthetic construct
KRLCLQYKGSKVCFRL

SEQ ID NO: 45 - protein, 16 aa, synthetic construct
RMRCLTYRGRRVCLEL

SEQ ID NO: 46 - protein, 16 aa, synthetic construct
SMRCLQYRGSRVCLTL

SEQ ID NO: 47 - protein, 16 aa, synthetic construct
HLRCLRYKGTRVCFSL

SEQ ID NO: 48 - protein, 16 aa, synthetic construct
HVRCLSYKGREVCVQL

SEQ ID NO: 49 - protein, 17 aa, synthetic construct
PRMCLFIYKGRRVCIPY

SEQ ID NO: 50 - protein, 16 aa, synthetic construct
HMRCLHYKGRRVCFLL

SEQ ID NO: 51 - protein, 16 aa, synthetic construct
HKRCLHYRGRMVCFLI

SEQ ID NO: 52 - protein, 16 aa, synthetic construct
QKRCLKYKGSRVCFFL

SEQ ID NO: 53 - protein, 16 aa, synthetic construct
HVRCLRYRGKNVCFLL

SEQ ID NO: 54 - protein, 16 aa, synthetic construct
SDVCLRYRGRPVCFQV

SEQ ID NO: 55 - protein, 16 aa, synthetic construct
RDVCLRYRGRPVCFQV

SEQ ID NO: 56 - protein, 16 aa, synthetic construct
HDVCLRYRGRPVCFQV

SEQ ID NO: 57 - protein, 16 aa, synthetic construct
SMVCLRYRGRPVCFQV

SEQ ID NO: 58 - protein, 16 aa, synthetic construct
SAVCLRYRGRPVCFQV

SEQ ID NO: 59 - protein, 16 aa, synthetic construct
SDVCLNYRGRPVCFQV

SEQ ID NO: 60 - protein, 16 aa, synthetic construct
SDVCLHYRGRPVCFQV

SEQ ID NO: 61 - protein, 16 aa, synthetic construct
SDVCLAYRGRPVCFQV

SEQ ID NO: 62 - protein, 16 aa, synthetic construct
SDVCLRYRGRPVCFAV

SEQ ID NO: 63 - protein, 16 aa, synthetic construct
SDVCLRYRGRPVCFQL

SEQ ID NO: 64 - protein, 16 aa, synthetic construct
SDVCLRYRGRPVCFQA

SEQ ID NO: 65 - protein, 14 aa, synthetic construct
HMVCLSYRGRPVCF

SEQ ID NO: 66 - protein, 15 aa, synthetic construct
HMVCLSYRGRPVCFS

SEQ ID NO: 67 - protein, 16 aa, synthetic construct
HQVCLSYRGQPVCFSL

SEQ ID NO: 68 - protein, 16 aa, synthetic construct
HQVCLSYRGRPTCFSL

SEQ ID NO: 69 - protein, 16 aa, synthetic construct
HQVCLSYRGRPVCYSL

SEQ ID NO: 70 - protein, 16 aa, synthetic construct
HQVCLSYRGQPVCFST

SEQ ID NO: 71 - protein, 16 aa, synthetic construct
HQVCLSYRGRPTCFST

SEQ ID NO: 72 - protein, 16 aa, synthetic construct
HQVCLSYRGQPTCFST

SEQ ID NO: 73 - protein, 16 aa, synthetic construct
  CROSSLNK 3..7: note = Tyr-Lys
  CROSSLNK 7..12: note = Lys-Lys
  CROSSLNK 11..16: note = Lys-Tyr
RTYRTYKRTYKKGRTY

SEQ ID NO: 74 - protein, 16 aa, synthetic construct
  SITE 1..16: note = D-amino acids
  CROSSLNK 3..7: note = Tyr-Lys
  CROSSLNK 11..16: note = Lys-Tyr
  CROSSLNK 7..12: note = Lys-Lys
RTYRTYKRTYKKGRTY

SEQ ID NO: 75 - protein, 16 aa, synthetic construct
  SITE 1..16: note = D-Amino Acids
  CROSSLNK 3..7: note = Tyr-Lys
  CROSSLNK 11..16: note = Lys-Tyr
  CROSSLNK 7..12: note = Lys-Lys
  MOD_RES 1: note = Phenylacetic acid
  MOD_RES 4: note = Phenylacetic acid
  MOD_RES 8: note = Phenylacetic acid
  MOD_RES 14: note = Phenylacetic acid
RTYRTYKRTYKKGRTY

SEQ ID NO: 76 - protein, 10 aa, synthetic construct
TWKTSRISIF

SEQ ID NO: 77 - protein, 10 aa, synthetic construct
FGRLVSSIRY

SEQ ID NO: 78 - protein, 13 aa, synthetic construct
  CROSSLNK 2..12: note = Cys-Cys
  SITE 13: note = C-Terminal Amide
DCAWHLGELVWCT

SEQ ID NO: 79 - protein, 12 aa, synthetic construct
DSAWHLGELWST

SEQ ID NO: 80 - protein, 13 aa, synthetic construct
DCHKRSFWADNCT

SEQ ID NO: 81 - protein, 13 aa, synthetic construct
DCRTQFRPNQTCT

SEQ ID NO: 82 - protein, 13 aa, synthetic construct
DCQLCDFWRTRCT

SEQ ID NO: 83 - protein, 13 aa, synthetic construct
DCFEDFNEQRTCT

SEQ ID NO: 84 - protein, 13 aa, synthetic construct
DCLAKFLKGKDCT

SEQ ID NO: 85 - protein, 13 aa, synthetic construct
DCWHRRTHKTFCT

SEQ ID NO: 86 - protein, 11 aa, synthetic construct
DCRTIQTRSCT

SEQ ID NO: 87 - protein, 13 aa, synthetic construct
DCIKLAQLHSVCT

SEQ ID NO: 88 - protein, 13 aa, synthetic construct
DCWRHRNATEWCT

SEQ ID NO: 89 - protein, 13 aa, synthetic construct
DCQNWIKDVHKCT

SEQ ID NO: 90 - protein, 13 aa, synthetic construct
DCAWHLGELVWCT

SEQ ID NO: 91 - protein, 13 aa, synthetic construct
DCAFHLGELVWCT

SEQ ID NO: 92 - protein, 13 aa, synthetic construct
DCAYHLGELVWCT

SEQ ID NO: 93 - protein, 11 aa, synthetic construct
  CROSSLNK 1..11: note = Pro-Pro
  SITE 11: note = D-Phenylalanine
PAWHLGELVWP

SEQ ID NO: 94 - protein, 15 aa, synthetic construct
  CROSSLNK 1..15: note = Pro-Pro
  CROSSLNK 3..13: note = Cys-Cys
  SITE 15: note = D-Amino Acid
PDCAWHLGELVWCTP

SEQ ID NO: 95 - protein, 15 aa, synthetic construct
  CROSSLNK 3..13: note = Cys-Cys
  CROSSLNK 1..15: note = Cys-Cys
CDCAWHLGELVWCTC

SEQ ID NO: 96 - protein, 12 aa, synthetic construct
EPIHRSTLTALL

SEQ ID NO: 97 - protein, 4 aa, synthetic construct
APAR

SEQ ID NO: 98 - protein, 6 aa, synthetic construct
  REPEAT 1..4: note = repeated twice
  CROSSLNK 4..5: note = His-Lys
  CROSSLNK 1: note = Cys-Cys
CFHHKG

SEQ ID NO: 99 - protein, 6 aa, synthetic construct
HWRGWV

SEQ ID NO: 100 - protein, 6 aa, synthetic construct
HYFKFD

SEQ ID NO: 101 - protein, 6 aa, synthetic construct
HFRRHL

SEQ ID NO: 102 - protein, 6 aa, synthetic construct
  NON_STD 3: note = Citrulline
HWXGWV

SEQ ID NO: 103 - protein, 6 aa, synthetic construct
  NON_STD 3: note = Citrulline
  MOD_RES 2: note = MeTrp
  MOD_RES 5: note = MeTrp
HWXGWV

SEQ ID NO: 104 - protein, 4 aa, synthetic construct
  REPEAT 1: note = repeated twice
  CROSSLNK 1..2: note = Asp-Ala
DAAG

SEQ ID NO: 105 - protein, 4 aa, synthetic construct
DAAG

SEQ ID NO: 106 - protein, 8 aa, synthetic construct
  NON_STD 1: note = (Nalpha-Ac)-S(A)
  NON_STD 8: note = Lact-E
  CROSSLNK 1..8: note = Nalpha-Ac-Lact-E
XRWHYFKX

SEQ ID NO: 107 - protein, 8 aa, synthetic construct
  CROSSLNK 1..8: note = Nalpha-Ac-Lact-E
  NON_STD 1: note = (Nalpha-Ac)-Dap(A)
  NON_STD 8: note = Lact-E
XRWHYFKX

SEQ ID NO: 108 - protein, 7 aa, synthetic construct
  CROSSLNK 1..7: note = Link M-Lys
  NON_STD 1: note = Link M
XWFRHYK

SEQ ID NO: 109 - protein, 8 aa, synthetic construct
NKFRGKYK

SEQ ID NO: 110 - protein, 8 aa, synthetic construct
NARKFYKG

SEQ ID NO: 111 - protein, 8 aa, synthetic construct
FYWHCLDE

SEQ ID NO: 112 - protein, 8 aa, synthetic construct
FYCHWALE

SEQ ID NO: 113 - protein, 8 aa, synthetic construct
FYCHTIDE

SEQ ID NO: 114 - protein, 16 aa, synthetic construct
FYWHCLDEFYCHTIDE

SEQ ID NO: 115 - protein, 4 aa, synthetic construct
RRGW

SEQ ID NO: 116 - protein, 7 aa, synthetic construct
KHRFNKD

SEQ ID NO: 117 - protein, 7 aa, synthetic construct
CPSTHWK

SEQ ID NO: 118 - protein, 7 aa, synthetic construct
NVQYFAV

SEQ ID NO: 119 - protein, 7 aa, synthetic construct
ASHTQKS

SEQ ID NO: 120 - protein, 7 aa, synthetic construct
QPQMSHM

SEQ ID NO: 121 - protein, 7 aa, synthetic construct
TNIESLK

SEQ ID NO: 122 - protein, 7 aa, synthetic construct
NCHKCWN

SEQ ID NO: 123 - protein, 7 aa, synthetic construct
SHLSKNF

SEQ ID NO: 124 - protein, 8 aa, synthetic construct
FDLLEHFY

SEQ ID NO: 125 - protein, 9 aa, synthetic construct
DLLHHFDYF

SEQ ID NO: 126 - protein, 9 aa, synthetic construct
  CROSSLNK 1..9: note = Cyclic [2-(3-Methylcyclohexyl)ethynyl]benzene Linker between Pro-1 and Ser-9
PFDLFIRKS

SEQ ID NO: 127 - protein, 9 aa, synthetic construct
  CROSSLNK 1..9: note = Cyclic 1-Methyl-4-[2-(4-methylcyclohexyl)ethynyl]benzene Linker between Pro-1 and Ser-9
PFDLFIRKS

SEQ ID NO: 128 - protein, 9 aa, synthetic construct
  CROSSLNK 1..9: note = Cyclic 1-(2-Cyclohexylethynyl)-3-methylbenzene Linker between Pro-1 and Ser-9
PFDLFIRKS

SEQ ID NO: 129 - protein, 9 aa, synthetic construct
  CROSSLNK 1..9: note = Cyclic 1-(2-Cyclohexylethynyl)-4-methylbenzene Linker between Pro-1 and Ser-9
PFDLFIRKS

SEQ ID NO: 130 - protein, 21 aa, synthetic construct
  DISULFID 3..19: note = Disulfide bond between Cys-3 and Cys-19
  DISULFID 7..15: note = Disulfide bond between Cys-7 and Cys-15
  MOD_RES 19: note = Amidation
VQCPHFCYELDYELCPDVCYV

SEQ ID NO: 131 - protein, 13 aa, synthetic construct
  MOD_RES 1: note = Amidation
  MOD_RES 13: note = Acetylation
  SITE 6: note = Lys-6 side chain links to Histidine
GYWYEKAWNRWAW

SEQ ID NO: 132 - protein, 13 aa, synthetic construct
  MOD_RES 1: note = Methylation
  MOD_RES 13: note = Acetylation
  SITE 6: note = Lys-6 side chain links to Histidine
GYWYEKAWNRWAW

SEQ ID NO: 133 - protein, 13 aa, synthetic construct
  MOD_RES 13: note = Methylation
  SITE 6: note = Lys-6 side chain links to Histidine
GYWYEKAWNRWAW

SEQ ID NO: 134 - protein, 13 aa, synthetic construct
GIFVIQSESLKKC

SEQ ID NO: 135 - protein, 9 aa, synthetic construct
SVLTLENCK

SEQ ID NO: 136 - protein, 8 aa, synthetic construct
SVLTLENC

SEQ ID NO: 137 - protein, 9 aa, synthetic construct
SVLTLDNCK

SEQ ID NO: 138 - protein, 8 aa, synthetic construct
SVLTEENC

SEQ ID NO: 139 - protein, 8 aa, synthetic construct
SVLTEENS

SEQ ID NO: 140 - protein, 8 aa, synthetic construct
SVLTDENC

SEQ ID NO: 141 - protein, 8 aa, synthetic construct
SVLTDENS

SEQ ID NO: 142 - protein, 9 aa, unidentified
PIQSESLKK

SEQ ID NO: 143 - protein, 9 aa, synthetic construct
VIDSESLKK

SEQ ID NO: 144 - protein, 9 aa, synthetic construct
PIDSESLKK

SEQ ID NO: 145 - protein, 9 aa, synthetic construct
VIQSESLKK

SEQ ID NO: 146 - protein, 17 aa, synthetic construct
  SITE 7: note = PEG
  SITE 9: note = PEG
PIESESXKXSVLTEENC

SEQ ID NO: 147 - protein, 17 aa, synthetic construct
  SITE 7: note = PEG
  SITE 9: note = PEG
VIQSESXKXSVLTLENC

SEQ ID NO: 148 - protein, 17 aa, synthetic construct
  SITE 7: note = PEG
  SITE 9: note = PEG
VIQSESXKXSVLTEENC

SEQ ID NO: 149 - protein, 17 aa, synthetic construct
  SITE 7: note = PEG
  SITE 9: note = PEG
PIDDESXKXSVLTLENC

SEQ ID NO: 150 - protein, 17 aa, synthetic construct
  SITE 7: note = PEG
  SITE 9: note = PEG
PIDDESXKXSVLTEENC

SEQ ID NO: 151 - protein, 19 aa, synthetic construct
VIQSESLKKCKSVLTLENC

SEQ ID NO: 152 - protein, 19 aa, synthetic construct
PIQSESLKKCKSVLTLENC

SEQ ID NO: 153 - protein, 19 aa, synthetic construct
VIESESLKKCKSVLTLENC

SEQ ID NO: 154 - protein, 19 aa, synthetic construct
VIDSESLKKCKSVLTLENC

SEQ ID NO: 155 - protein, 19 aa, synthetic construct
PIESESLKKCKSVLTLENC

SEQ ID NO: 156 - protein, 19 aa, synthetic construct
VIQSESLKKCIQAGKLENC

SEQ ID NO: 157 - protein, 19 aa, synthetic construct
PIQSESLKKCIQAGKLENC

SEQ ID NO: 158 - protein, 19 aa, synthetic construct
VIESESLKKCIQAGKLENC

SEQ ID NO: 159 - protein, 19 aa, synthetic construct
VIDSESLKKCIQAGKLENC

SEQ ID NO: 160 - protein, 19 aa, synthetic construct
PIESESLKKCIQAGKLENC

SEQ ID NO: 161 - protein, 19 aa, synthetic construct
PIQSESLKKCKSVLTLENK

SEQ ID NO: 162 - protein, 19 aa, synthetic construct
VIESESLKKCKSVLTLENK

SEQ ID NO: 163 - protein, 19 aa, synthetic construct
VIDSESLKKCKSVLTLENK

SEQ ID NO: 164 - protein, 19 aa, synthetic construct
PIESESLKKCKSVLTLENK

SEQ ID NO: 165 - protein, 19 aa, synthetic construct
VIQSESLKKCIQAGKLENK

SEQ ID NO: 166 - protein, 19 aa, synthetic construct
PIQSESLKKCIQAGKLENK

SEQ ID NO: 167 - protein, 19 aa, synthetic construct
VIESESLKKCIQAGKLENK

SEQ ID NO: 168 - protein, 19 aa, synthetic construct
VIDSESLKKCIQAGKLENK

SEQ ID NO: 169 - protein, 19 aa, synthetic construct
PIESESLKKCIQAGKLENK

SEQ ID NO: 170 - protein, 16 aa, synthetic construct
PIESESGSVLTLENCK

SEQ ID NO: 171 - protein, 17 aa, synthetic construct
PIESESGGSVLTLENCK

SEQ ID NO: 172 - protein, 18 aa, synthetic construct
PIESESGGGSVLTLENCK

SEQ ID NO: 173 - protein, 19 aa, synthetic construct
PIESESGGGGSVLTLENCK

SEQ ID NO: 174 - protein, 20 aa, synthetic construct
PIESESGGGGGSVLTLENCK

SEQ ID NO: 175 - protein, 16 aa, synthetic construct
VIQSESGSVLTLENCK

SEQ ID NO: 176 - protein, 17 aa, synthetic construct
VIQSESGGSVLTLENCK

SEQ ID NO: 177 - protein, 18 aa, synthetic construct
VIQSESGGGSVLTLENCK

SEQ ID NO: 178 - protein, 19 aa, synthetic construct
VIQSESGGGGSVLTLENCK

SEQ ID NO: 179 - protein, 20 aa, synthetic construct
VIQSESGGGGGSVLTLENCK

SEQ ID NO: 180 - protein, 28 aa, synthetic construct
KGCFVIQSESLKKSIQAGKSVLTLENCK

SEQ ID NO: 181 - protein, 21 aa, synthetic construct
LKKCIQAGKSVLTLENCKQAN

SEQ ID NO: 182 - protein, 22 aa, synthetic construct
WQDKGIFVIQSESLKKCIQAGK

SEQ ID NO: 183 - protein, 28 aa, synthetic construct
KGIFVIQSESLKKCIQAGKSVLTLENCK

SEQ ID NO: 184 - protein, 13 aa, synthetic construct
GIFVIQSESLKKC

SEQ ID NO: 185 - protein, 10 aa, synthetic construct
WSVLTLENCK

SEQ ID NO: 186 - protein, 31 aa, synthetic construct
WQDKGIFVIQSESLKKCIQAGKSVLTLENCK

SEQ ID NO: 187 - protein, 9 aa, synthetic construct
YDWIPSSAW

SEQ ID NO: 188 - protein, 9 aa, synthetic construct
AGAIWQRDW

SEQ ID NO: 189 - protein, 9 aa, synthetic construct
AGAIWQKDW

SEQ ID NO: 190 - protein, 8 aa, synthetic construct
VIQSESLK

SEQ ID NO: 191 - protein, 8 aa, synthetic construct
PIQSESLK

SEQ ID NO: 192 - protein, 8 aa, synthetic construct
PIESESLK

SEQ ID NO: 193 - protein, 9 aa, synthetic construct
SVLTEENCK

SEQ ID NO: 194 - protein, 8 aa, synthetic construct
  SITE 5: note = Any Naturally-Occurring Amino Acid
  SITE 8: note = Any Naturally-Occurring Amino Acid
SVLTXENX

SEQ ID NO: 195 - protein, 9 aa, synthetic construct
  SITE 5: note = Any Naturally-Occurring Amino Acid
SVLTXENCK

SEQ ID NO: 196 - protein, 8 aa, synthetic construct
  SITE 1: note = Any Naturally-Occurring Amino Acid
  SITE 3: note = Any Naturally-Occurring Amino Acid
  SITE 4: note = Any Naturally-Occurring Amino Acid
  SITE 6: note = Any Naturally-Occurring Amino Acid
XIXXEXLK

SEQ ID NO: 197 - protein, 14 aa, synthetic construct
  MOD_RES 5: note = N-Methylation
  MOD_RES 14: note = N-Methylation
  DISULFID 3..13: note = Cys-Cys
  SITE 1: note = D-Amino Acid
  MOD_RES 9: note = MeGly
YICVWQDWGAHRCI

SEQ ID NO: 198 - protein, 26 aa, synthetic construct
  DISULFID 2..12: note = Cys-Cys
ICVVQDWGHHRCTAGMANLTSHASAI

SEQ ID NO: 199 - protein, 13 aa, synthetic construct
  DISULFID 2..12: note = Cys-Cys
ICVVQDWGHHRCT

SEQ ID NO: 200 - protein, 11 aa, synthetic construct
  DISULFID 1..11: note = Cys-Cys
CVVQDWGHHAC

SEQ ID NO: 201 - protein, 13 aa, synthetic construct
  DISULFID 2..12: note = Cys-Cys
  SITE 1: note = N-Terminal Acetyl
  SITE 13: note = C-Terminal Amide
ICVVQDWGHHRCT

SEQ ID NO: 202 - protein, 12 aa, synthetic construct
  DISULFID 1..11: note = Cys-Cys
  SITE 12: note = C-Terminal Amide
CVVQDWGHHRCT

SEQ ID NO: 203 - protein, 11 aa, synthetic construct
  DISULFID 1..11: note = Cys-Cys
  SITE 11: note = C-Terminal Amide
CVVQDWGHHRC

SEQ ID NO: 204 - protein, 13 aa, synthetic construct
  SITE 1: note = N-Terminal Acetyl
  DISULFID 2..12: note = Cys-Cys
  SITE 13: note = C-Terminal Amide
ICVVGDWGHHRCT

SEQ ID NO: 205 - protein, 13 aa, synthetic construct
  SITE 1: note = N-Terminal Acetyl
  DISULFID 2..12: note = Cys-Cys
  SITE 13: note = C-Terminal Amide
ICVVQPWGHHRCT

SEQ ID NO: 206 - protein, 17 aa, synthetic construct
  DISULFID 6..16: note = Cys-Cys
  SITE 1: note = N-Terminal Biotin
  SITE 17: note = C-Terminal Amide
KYSSICVVQDWGHHRCT

SEQ ID NO: 207 - protein, 27 aa, synthetic construct
  SITE 1: note = N-Terminal Acetyl
  DISULFID 2..12: note = Cys-Cys
  SITE 27: note = C-Terminal Biotin
ICVVQDWGHHRCTAGHMANLTSHASAK

SEQ ID NO: 208 - protein, 13 aa, synthetic construct
  SITE 1: note = N-Terminal Acetyl
  MOD_RES 4: note = N-Methylation
ICVWQDWGAHRCT

SEQ ID NO: 209 - protein, 14 aa, synthetic construct
  MOD_RES 9: note = MeGly
  MOD_RES 14: note = N-Methylation
  MOD_RES 5: note = 1-Methylation
YICVWQDWGAHRCI

SEQ ID NO: 210 - protein, 14 aa, synthetic construct
  SITE 1: note = PEG40K
  DISULFID 3..13: note = Cys-Cys
  MOD_RES 5: note = N-Methylation
  MOD_RES 9: note = MeGly
  MOD_RES 14: note = N-Methylation
YICVWQDWGAHRCI

SEQ ID NO: 211 - protein, 15 aa, synthetic construct
  SITE 1: note = N-Terminal Acetyl
  MOD_RES 5: note = N-Methylation
  MOD_RES 9: note = MeGly
  MOD_RES 14: note = N-Methylation
  DISULFID 3..13: note = Cys-Cys
YICVWQDWGAHRCIK

SEQ ID NO: 212 - protein, 15 aa, synthetic construct
  SITE 1: note = N-Terminal Acetyl
  DISULFID 3..13: note = Cys-Cys
  MOD_RES 5: note = N-Methylation
  MOD_RES 9: note = MeGly
  SITE 15: note = C-terminal PEG40K
YICVWQDWGAHRCIK

SEQ ID NO: 213 - protein, 13 aa, synthetic construct
  DISULFID 2..12: note = Cys-Cys
  SITE 1: note = N-Terminal Acetyl
  SITE 13: note = C-Terminal Amide
ICVWQDWGAHRCT

SEQ ID NO: 214 - protein, 13 aa, synthetic construct
  DISULFID 2..12: note = Cys-Cys
  SITE 1: note = N-Terminal Acetyl
  MOD_RES 5: note = N-Methylation
  MOD_RES 8: note = MeGly
  SITE 13: note = C-Terminal Amide
ICVWQDWGAHRCI

SEQ ID NO: 215 - protein, 13 aa, synthetic construct
  SITE 1: note = N-Terminal Acetyl
  MOD_RES 4: note = N-Methylation
  MOD_RES 8: note = MeGly
  MOD_RES 13: note = N-Methylation
  DISULFID 2..12: note = Cys-Cys
ICVWQDWGAHRCI

SEQ ID NO: 216 - protein, 13 aa, synthetic construct
  DISULFID 2..12: note = Cys-Cys
  SITE 1: note = N-Terminal Acetyl
  MOD_RES 4: note = N-Methylation
  SITE 13: note = C-Terminal Amide
ICVWQDWGAHRCT

SEQ ID NO: 217 - protein, 13 aa, synthetic construct
  DISULFID 2..13: note = Cys-Cys
  SITE 1: note = N-Terminal Acetyl
XCVXQDWGXXXCT

SEQ ID NO: 218 - protein, 13 aa, synthetic construct
  DISULFID 2..12: note = Cys-Cys
  SITE 1: note = N-Terminal Acetyl
  SITE 13: note = C-Terminal Amide
ICVVQDWGHHRCT

SEQ ID NO: 219 - protein, 13 aa, synthetic construct
  SITE 1: note = N-Terminal Acetyl
  DISULFID 2..12: note = Cys-Cys
  SITE 13: note = C-Terminal Amide
ICVVQDWGAHRCT

SEQ ID NO: 220 - protein, 13 aa, synthetic construct
  SITE 1: note = N-Terminal Acetyl
  DISULFID 2..12: note = Cys-Cys
  SITE 13: note = C-Terminal Amide
ICVTQDWGHHRCT

SEQ ID NO: 221 - protein, 13 aa, synthetic construct
  SITE 1: note = N-Terminal Acetyl
  DISULFID 2..12: note = Cys-Cys
  SITE 13: note = C-Terminal Amide
ICVSQDWGHHRCT

SEQ ID NO: 222 - protein, 13 aa, synthetic construct
  SITE 1: note = N-Terminal Acetyl
  DISULFID 2..12: note = Cys-Cys
  SITE 13: note = C-Terminal Amide
ICVHQDWGHHRCT

SEQ ID NO: 223 - protein, 13 aa, synthetic construct
  SITE 1: note = N-Terminal Acetyl
  DISULFID 2..12: note = Cys-Cys
  SITE 13: note = C-Terminal Amide
ICVFQDWGHHRCT

SEQ ID NO: 224 - protein, 13 aa, synthetic construct
  SITE 1: note = N-Terminal Acetyl
  DISULFID 2..12: note = Cys-Cys
  SITE 13: note = C-Terminal Amide
ICVYQDWGAHRCT

SEQ ID NO: 225 - protein, 13 aa, synthetic construct
  SITE 1: note = N-Terminal Acetyl
  DISULFID 2..12: note = Cys-Cys
  SITE 13: note = C-Terminal Amide
ICVWQDWGWHRCT

SEQ ID NO: 226 - protein, 13 aa, synthetic construct
  SITE 1: note = N-Terminal Acetyl
  DISULFID 2..12: note = Cys-Cys
  SITE 13: note = C-Terminal Amide
ICVWQDWGHHRCT

SEQ ID NO: 227 - protein, 13 aa, synthetic construct
  SITE 1: note = N-Terminal Acetyl
  DISULFID 2..12: note = Cys-Cys
ICVWQDWGAHRCT

SEQ ID NO: 228 - protein, 13 aa, synthetic construct
  SITE 1: note = N-Terminal Acetyl
  DISULFID 2..12: note = Cys-Cys
  SITE 13: note = C-Terminal Amide
ICVWQDWGAHRCT

SEQ ID NO: 229 - protein, 13 aa, synthetic construct
  SITE 1: note = N-Terminal Acetyl
  DISULFID 2..12: note = Cys-Cys
  SITE 10: note = D-Amino Acid
ICVWQDWGAHRCT

SEQ ID NO: 230 - protein, 13 aa, synthetic construct
  SITE 1: note = N-Terminal Acetyl
  DISULFID 2..12: note = Cys-Cys
  SITE 9: note = D-Amino Acid
ICVWQDWGAHRCT

SEQ ID NO: 231 - protein, 13 aa, synthetic construct
  SITE 1: note = D-Amino Acid, N-Terminal Acetyl
  DISULFID 2..12: note = Cys-Cys
ICVWQDWGAHRCT

SEQ ID NO: 232 - protein, 13 aa, synthetic construct
  DISULFID 2..12: note = Cys-Cys
  SITE 1: note = N-Terminal Acetyl
  SITE 13: note = D-Amino Acid
ICVWQDWGAHRCT

SEQ ID NO: 233 - protein, 13 aa, synthetic construct
  DISULFID 2..12: note = Cys-Cys
  SITE 1: note = N-Terminal Acetyl
  SITE 13: note = C-Terminal Amide
ICVWQDWGAHRCT

SEQ ID NO: 234 - protein, 13 aa, synthetic construct
  DISULFID 2..12: note = Cys-Cys
  SITE 13: note = C-Terminal Amide
WCVWQDWGTNRCW

SEQ ID NO: 235 - protein, 13 aa, synthetic construct
  SITE 1: note = N-Terminal Acetyl
  DISULFID 2..12: note = Cys-Cys
  SITE 13: note = C-Terminal Amide
DCVWQDWGTNKCW

SEQ ID NO: 236 - protein, 13 aa, synthetic construct
  DISULFID 2..12: note = Cys-Cys
  SITE 13: note = C-Terminal Amide
QCVWQDWGQNQCW

SEQ ID NO: 237 - protein, 13 aa, synthetic construct
  DISULFID 2..12: note = Cys-Cys
  SITE 1: note = N-Terminal Acetyl
  SITE 13: note = C-Terminal Amide
ICVWQDWGAHRCW

SEQ ID NO: 238 - protein, 13 aa, synthetic construct
  SITE 1: note = N-Terminal Acetyl
  DISULFID 2..12: note = Cys-Cys
  SITE 13: note = C-Terminal Amide
WCVWQDWGAHRCT

SEQ ID NO: 239 - protein, 13 aa, synthetic construct
  SITE 1: note = N-Terminal Acetyl
  DISULFID 2..12: note = Cys-Cys
  SITE 13: note = C-Terminal Amide
WCVWQDWGAHRCW

SEQ ID NO: 240 - protein, 13 aa, synthetic construct
  SITE 12: note = Homocysteine
  SITE 1: note = N-Terminal Acetyl
  CROSSLNK 2..12: note = Ala-Hcy
  SITE 13: note = C-Terminal Amide
IAVWQDWGAHRXT

SEQ ID NO: 241 - protein, 13 aa, synthetic construct
  SITE 1: note = N-Terminal Acetyl
  DISULFID 2..12: note = Cys-Cys
  MOD_RES 13: note = N-Methylation
  SITE 13: note = C-Terminal Amide
ICVWQDWGAHRCI

SEQ ID NO: 242 - protein, 13 aa, synthetic construct
  SITE 1: note = N-Terminal Acetyl
  DISULFID 2..12: note = Ala-Hcy
  MOD_RES 13: note = N-Methylation
  SITE 12: note = Homocysteine
  SITE 13: note = C-Terminal Amide
IAVWQDWGAHRXI

SEQ ID NO: 243 - protein, 13 aa, synthetic construct
  SITE 1: note = N-Terminal Acetyl
  MOD_RES 4: note = 5-Fluoro-L-tryptophan
  DISULFID 2..12: note = Cys-Cys
  SITE 13: note = C-Terminal Amide
ICVWQDWGAHRCT

SEQ ID NO: 244 - protein, 13 aa, synthetic construct
  SITE 1: note = N-Terminal Acetyl
  MOD_RES 4: note = 5-Methylation
  SITE 13: note = C-Terminal Amide
  DISULFID 2..12: note = Cys-Cys
ICVWQDWGAHRCT

SEQ ID NO: 245 - protein, 13 aa, synthetic construct
  SITE 4: note = Naphthylalanine
  SITE 1: note = N-Terminal Acetyl
  DISULFID 2..12: note = Cys-Cys
ICVXQDWGAHRCT

SEQ ID NO: 246 - protein, 13 aa, synthetic construct
  SITE 1: note = N-Terminal Acetyl
  MOD_RES 7: note = 5-Fluoro-L-tryptophan
  SITE 13: note = C-Terminal Acetyl
  DISULFID 2..12: note = Cys-Cys
ICVWQDWGAHRCT

SEQ ID NO: 247 - protein, 13 aa, synthetic construct
  SITE 1: note = N-Terminal Acetyl
  MOD_RES 7: note = 5-Methylation
  SITE 13: note = C-Terminal Amide
  DISULFID 2..12: note = Cys-Cys
ICVWQDWGAHRCT

SEQ ID NO: 248 - protein, 13 aa, synthetic construct
  SITE 1: note = N-Terminal Acetyl
  MOD_RES 7: note = 1-Methylation
  SITE 13: note = C-Terminal Amide
  DISULFID 2..12: note = Cys-Cys
ICVWQDWGAHRCT

SEQ ID NO: 249 - protein, 13 aa, synthetic construct
  SITE 1: note = N-Terminal Acetyl
  DISULFID 2..12: note = Cys-Cys
  SITE 13: note = C-Terminal Amide
ICVYQDWGAHRCT

SEQ ID NO: 250 - protein, 13 aa, synthetic construct
  SITE 1: note = N-Terminal Acetyl
  DISULFID 2..12: note = Cys-Cys
ICVWQDWGAHRCT

SEQ ID NO: 251 - protein, 13 aa, synthetic construct
  SITE 1: note = N-Terminal Acetyl
  DISULFID 2..12: note = Cys-Cys
ICVWQDWGAHRCT

SEQ ID NO: 252 - protein, 13 aa, synthetic construct
  SITE 1: note = N-Terminal Acetyl
  SITE 13: note = D-Amino Acid
  DISULFID 2..12: note = Cys-Cys
ICVWQDWGAHRCT

SEQ ID NO: 253 - protein, 13 aa, synthetic construct
  SITE 1: note = N-Terminal Acetyl
  SITE 4: note = Naphthylalanine
  DISULFID 2..12: note = Cys-Cys
ICVXQDWGAHRCT

SEQ ID NO: 254 - protein, 13 aa, synthetic construct
  SITE 1: note = N-Terminal Acetyl
  SITE 4: note = Naphthylalanine
  DISULFID 2..12: note = Cys-Cys
ICVXQDWGAHRCT

SEQ ID NO: 255 - protein, 13 aa, synthetic construct
  SITE 1: note = N-Terminal Acetyl
  SITE 4: note = Naphthylalanine
  DISULFID 2..12: note = Cys-Cys
ICVXQDWGAHRCT

SEQ ID NO: 256 - protein, 13 aa, synthetic construct
  SITE 1: note = N-Terminal Acetyl
  SITE 4: note = Lysoalanine
  DISULFID 2..12: note = Cys-Cys
ICVXQDWGAHRCT

SEQ ID NO: 257 - protein, 13 aa, synthetic construct
  SITE 1: note = N-Terminal Acetyl
  SITE 4: note = Lysoalanine
  DISULFID 2..12: note = Cys-Cys
ICVXQDWGAHRCT

SEQ ID NO: 258 - protein, 13 aa, synthetic construct
  SITE 1: note = N-Terminal Acetyl
  MOD_RES 4: note = dihydrotryptophan
  DISULFID 2..12: note = Cys-Cys
ICVXQDWGAHRCT

SEQ ID NO: 259 - protein, 13 aa, synthetic construct
  SITE 1: note = N-Terminal Acetyl
  SITE 4: note = 4-Benzoyl-L-phenyl alanine
  DISULFID 2..12: note = Cys-Cys
ICVXQDWGAHRCT

SEQ ID NO: 260 - protein, 13 aa, synthetic construct
  SITE 1: note = N-Terminal Acetyl
  SITE 4: note = 4-Benzoylphenylalanine
  DISULFID 2..12: note = Cys-Cys
ICVXQDWGAHRCT

SEQ ID NO: 261 - protein, 13 aa, synthetic construct
  SITE 1: note = N-Terminal Acetyl
  SITE 4: note = L-3-Benzothienylalanine
  DISULFID 2..12: note = Cys-Cys
ICVXQDWGAHRCT

SEQ ID NO: 262 - protein, 13 aa, synthetic construct
  SITE 1: note = N-Terminal Acetyl
  SITE 4: note = L-3-Benzothienylalanine
  DISULFID 2..12: note = Cys-Cys
ICVXQDWGAHRCT

SEQ ID NO: 263 - protein, 13 aa, synthetic construct
  SITE 1: note = N-Terminal Acetyl
  SITE 9: note = Abu
  DISULFID 2..12: note = Cys-Cys
ICVWQDWGXHRCT

SEQ ID NO: 264 - protein, 16 aa, synthetic construct
  MOD_RES 1: note = H-Gly
  DISULFID 3..13: note = Cys-Cys
GICVWQDWGAHRCTAN

SEQ ID NO: 265 - protein, 13 aa, synthetic construct
  SITE 1: note = N-Terminal Acetyl
  MOD_RES 4: note = 5-Fluoro-L-tryptophan
  DISULFID 2..12: note = Cys-Cys
ICVWQDWGAHRCT

SEQ ID NO: 266 - protein, 13 aa, synthetic construct
  SITE 1: note = N-Terminal Acetyl
  MOD_RES 4: note = 5-Methyl-L-tryptophan
  DISULFID 2..12: note = Cys-Cys
ICVWQDWGAHRCT

SEQ ID NO: 267 - protein, 13 aa, synthetic construct
  SITE 1: note = N-Terminal Acetyl
  MOD_RES 4: note = 1-Methyl-L-tryptophan
  DISULFID 2..12: note = Cys-Cys
ICVWQDWGAHRCT

SEQ ID NO: 268 - protein, 13 aa, synthetic construct
  SITE 1: note = N-Terminal Acetyl
  MOD_RES 7: note = 5-Fluoro-L-tryptophan
  DISULFID 2..12: note = Cys-Cys
ICVWQDWGAHRCT

SEQ ID NO: 269 - protein, 13 aa, synthetic construct
  SITE 1: note = N-Terminal Acetyl
  MOD_RES 4: note = 5-Fluoro-L-tryptophan
  MOD_RES 7: note = 5-Fluoro-L-tryptophan
  DISULFID 2..12: note = Cys-Cys
ICVWQDWGAHRCT

SEQ ID NO: 270 - protein, 13 aa, synthetic construct
  SITE 1: note = N-Terminal Acetyl
  MOD_RES 4: note = 5-Methyl-L-tryptophan
  MOD_RES 7: note = 5-Fluoro-L-tryptophan
  DISULFID 2..12: note = Cys-Cys
ICVWQDWGAHRCT

SEQ ID NO: 271 - protein, 13 aa, synthetic construct
  SITE 1: note = N-Terminal Acetyl
  MOD_RES 4: note = 1-Methyl-L-tryptophan
  MOD_RES 7: note = 5-Fluoro-L-tryptophan
  DISULFID 2..12: note = Cys-Cys
ICVWQDWGAHRCT

SEQ ID NO: 272 - protein, 15 aa, synthetic construct
  SITE 1: note = H-Gly, N-Terminal Acetyl
  MOD_RES 4: note = 6-Fluorotryptophan
  MOD_RES 7: note = 6-Fluorotryptophan
  DISULFID 2..12: note = Cys-Cys
GICVWQDWGAHRCTN

SEQ ID NO: 273 - protein, 13 aa, synthetic construct
  SITE 1: note = N-Terminal Acetyl
  MOD_RES 4: note = 1-Formyl-L-tryptophan
  DISULFID 2..12: note = Cys-Cys
ICVWQDWGAHRCT

SEQ ID NO: 274 - protein, 13 aa, synthetic construct
  SITE 1: note = N-Terminal Acetyl
  MOD_RES 4: note = 1-methoxy-Tryptophan
  DISULFID 2..12: note = Cys-Cys
ICVWQDWGAHRCT

SEQ ID NO: 275 - protein, 15 aa, synthetic construct
  MOD_RES 1: note = H-Gly
  MOD_RES 5: note = 5-Fluoro-L-tryptophan
  MOD_RES 8: note = 5-Fluoro-L-tryptophan
  DISULFID 3..13: note = Cys-Cys
GICVWQDWGAHRCTN

SEQ ID NO: 276 - protein, 13 aa, synthetic construct
  DISULFID 2..12: note = Cys-Cys
  SITE 1: note = N-Terminal Acetyl
  MOD_RES 4: note = Methylation
  SITE 13: note = C-terminal 8-Amino-3,6-dioxaoctanoic acid
ICVWQDWGAHRCT

SEQ ID NO: 277 - protein, 7 aa, synthetic construct
  SITE 1: note = N-Terminal Acetyl
AEAKAKA

SEQ ID NO: 278 - protein, 15 aa, synthetic construct
IALILEPICCQERAA

SEQ ID NO: 279 - protein, 15 aa, synthetic construct
  SITE 15: note = C-Terminal dPEG24
IALILEPICCQERAA

SEQ ID NO: 280 - protein, 15 aa, synthetic construct
  SITE 1: note = N-Terminal dPEG24
IALILEPICCQERAA

SEQ ID NO: 281 - protein, 15 aa, synthetic construct
RALILEPICCQERAA

SEQ ID NO: 282 - protein, 15 aa, synthetic construct
IRLILEPICCQERAA

SEQ ID NO: 283 - protein, 15 aa, synthetic construct
IARILEPICCQERAA

SEQ ID NO: 284 - protein, 15 aa, synthetic construct
IALIREPICCQERAA

SEQ ID NO: 285 - protein, 15 aa, synthetic construct
IALILEPICCRERAA

SEQ ID NO: 286 - protein, 15 aa, synthetic construct
IALILEPICCQRRAA

SEQ ID NO: 287 - protein, 15 aa, synthetic construct
IELILEPICCQERAA

SEQ ID NO: 288 - protein, 15 aa, synthetic construct
IAEILEPICCQERAA

SEQ ID NO: 289 - protein, 15 aa, synthetic construct
IALILEPICCQEEAA

SEQ ID NO: 290 - protein, 15 aa, synthetic construct
IALILEPICCQEREA

SEQ ID NO: 291 - protein, 15 aa, synthetic construct
IALILEEICCQERAA

SEQ ID NO: 292 - protein, 15 aa, synthetic construct
IALILEPECCQERAA

SEQ ID NO: 293 - protein, 30 aa, synthetic construct
PAICQRATATLGTVGSNTSGTTAIEACILL

SEQ ID NO: 294 - protein, 15 aa, synthetic construct
  DISULFID 1..12: note = Cys-Cys
CEGPFGPRHDLTFCW

SEQ ID NO: 295 - protein, 14 aa, synthetic construct
  SITE 1: note = N-terminal bromoacetyl
  CROSSLNK 1..13: note = peptide is cyclized through a reaction of N-terminal bromoacetyl moiety with the side chain of cysteine at position 13
EGPFGPRHDLTFCW

SEQ ID NO: 296 - protein, 7 aa, synthetic construct
QYYPFSX

SEQ ID NO: 297 - protein, 7 aa, synthetic construct
NPFNLAR

SEQ ID NO: 298 - protein, 12 aa, synthetic construct
QLQDMTSSPFWL

SEQ ID NO: 299 - protein, 12 aa, synthetic construct
NPFVIGRWHPPH

SEQ ID NO: 300 - protein, 12 aa, synthetic construct
SLAKFLNPFLYR

SEQ ID NO: 301 - protein, 12 aa, synthetic construct
ASTPRFEPFQLD

SEQ ID NO: 302 - protein, 12 aa, synthetic construct
SLHSQPYSPFML

SEQ ID NO: 303 - protein, 12 aa, synthetic construct
NILSSWSSPFVF

SEQ ID NO: 304 - protein, 12 aa, synthetic construct
NLPSSWTNPFYL

SEQ ID NO: 305 - protein, 7 aa, synthetic construct
SPFMLHP

SEQ ID NO: 306 - protein, 7 aa, synthetic construct
PSPFMLT

SEQ ID NO: 307 - protein, 7 aa, synthetic construct
IGPFHLH

SEQ ID NO: 308 - protein, 7 aa, synthetic construct
TNPFMLN

SEQ ID NO: 309 - protein, 7 aa, synthetic construct
NTTFLYP

SEQ ID NO: 310 - protein, 7 aa, synthetic construct
SHYTQYL

SEQ ID NO: 311 - protein, 7 aa, synthetic construct
NHHPNYW

SEQ ID NO: 312 - protein, 7 aa, synthetic construct
VHYPLSW

SEQ ID NO: 313 - protein, 12 aa, synthetic construct
HHLKYSDTSPPI

SEQ ID NO: 314 - protein, 13 aa, synthetic construct
SHMHERWDTSPPI

SEQ ID NO: 315 - protein, 12 aa, synthetic construct
SHMHERWDTSYQ

SEQ ID NO: 316 - protein, 12 aa, synthetic construct
SHIHSNAAWRIT

SEQ ID NO: 317 - protein, 7 aa, synthetic construct
WHYPHWQ

SEQ ID NO: 318 - protein, 7 aa, synthetic construct
SHYLYTQ

SEQ ID NO: 319 - protein, 7 aa, synthetic construct
AHYSFTQ

SEQ ID NO: 320 - protein, 7 aa, synthetic construct
THYPTFY

SEQ ID NO: 321 - protein, 7 aa, synthetic construct
EHNTSFW

SEQ ID NO: 322 - protein, 7 aa, synthetic construct
NHYKLTW

SEQ ID NO: 323 - protein, 7 aa, synthetic construct
NHSPYFQ

SEQ ID NO: 324 - protein, 7 aa, synthetic construct
SHYQHYQ

SEQ ID NO: 325 - protein, 30 aa, synthetic construct
PAICQRATATLGTVGSNTSGTTEIEACILL

SEQ ID NO: 326 - protein, 10 aa, synthetic construct
WLGLGGGYGW

SEQ ID NO: 327 - protein, 15 aa, synthetic construct
FYGPFFLNDSLRGIW

SEQ ID NO: 328 - protein, 15 aa, synthetic construct
LRFLNPFSLDGSGFW

SEQ ID NO: 329 - protein, 15 aa, synthetic construct
HSPFCLGVLECFGLV

SEQ ID NO: 330 - protein, 15 aa, synthetic construct
TCGAFYLYHDPFICG

SEQ ID NO: 331 - protein, 15 aa, synthetic construct
MQHCLASHELYLPWC

SEQ ID NO: 332 - protein, 15 aa, synthetic construct
FFVFGSGDAFAFSDM

SEQ ID NO: 333 - protein, 15 aa, synthetic construct
PCVIIDTGSSRWCYL

SEQ ID NO: 334 - protein, 15 aa, synthetic construct
HSPFCLGVLECFGLV

SEQ ID NO: 335 - protein, 15 aa, synthetic construct
HAAFEPRGDVRHTLL

SEQ ID NO: 336 - protein, 12 aa, synthetic construct
CRWDGSWGEVRC

SEQ ID NO: 337 - protein, 12 aa, synthetic construct
CYWVGTWGEAVC

SEQ ID NO: 338 - protein, 16 aa, synthetic construct
RWFPCPNKEGCCSISV

SEQ ID NO: 339 - protein, 16 aa, synthetic construct
RSTYCNKNKDSCHIPE

SEQ ID NO: 340 - protein, 16 aa, synthetic construct
QPPQCIKDGGFVICRV

SEQ ID NO: 341 - protein, 16 aa, synthetic construct
KGKKCKPEEHPCNEPM

SEQ ID NO: 342 - protein, 16 aa, synthetic construct
NKMTCSDDGKLCWEHL

SEQ ID NO: 343 - protein, 14 aa, synthetic construct
PLGRPCPTCPLAPS

SEQ ID NO: 344 - protein, 14 aa, synthetic construct
QRMRPCPSCPLAPW

SEQ ID NO: 345 - protein, 14 aa, synthetic construct
WPSRPCPSCPEVPP

SEQ ID NO: 346 - protein, 14 aa, synthetic construct
SCTKDCPTCPLVPV

SEQ ID NO: 347 - protein, 30 aa, synthetic construct
PAIAQRATATLGTVGSNTSGTTEIEACILL

SEQ ID NO: 348 - protein, 30 aa, synthetic construct
PAICQRATATLGTVGSNTSGTTEIEAAILL

SEQ ID NO: 349 - protein, 30 aa, synthetic construct; identical to SEQ ID NO: 293

SEQ ID NO: 350 - protein, 30 aa, synthetic construct
PAICQRATATLGTVGSNTSGTTEIAACILL

SEQ ID NO: 351 - protein, 15 aa, synthetic construct
PAICQRAEIEACILL

SEQ ID NO: 352 - protein, 15 aa, synthetic construct
PAIAQRAEIEAAILL

SEQ ID NO: 353 - protein, 27 aa, synthetic construct
PAICQRATATLGTNTSGTTEIEACILL

SEQ ID NO: 354 - protein, 23 aa, synthetic construct
PAICQRATATLSGTTEIEACILL

SEQ ID NO: 355 - protein, 19 aa, synthetic construct
PAICQRATATTEIEACILL

SEQ ID NO: 356 - protein, 29 aa, synthetic construct
AICQRATATLGTVGSNTSGTTEIEACILL

SEQ ID NO: 357 - protein, 28 aa, synthetic construct
ICQRATATLGTVGSNTSGTTEIEACILL

SEQ ID NO: 358 - protein, 27 aa, synthetic construct
CQRATATLGTVGSNTSGTTEIEACILL

SEQ ID NO: 359 - protein, 29 aa, synthetic construct
PAICQRATATLGTVGSNTSGTTEIEACIL

SEQ ID NO: 360 - protein, 28 aa, synthetic construct
PAICQRATATLGTVGSNTSGTTEIEACI

SEQ ID NO: 361 - protein, 27 aa, synthetic construct
PAICQRATATLGTVGSNTSGTTEIEAC

SEQ ID NO: 362 - protein, 15 aa, synthetic construct
  SITE 1: note = N-Terminal Acetyl
IALILEPICCQERAA

SEQ ID NO: 363 - protein, 30 aa, synthetic construct; identical to SEQ ID NO: 325
  SITE 1: note = N-Terminal Acetyl

SEQ ID NO: 364 - protein, 30 aa, synthetic construct; identical to SEQ ID NO: 347
  SITE 1: note = N-Terminal Acetyl

SEQ ID NO: 365 - protein, 30 aa, synthetic construct; identical to SEQ ID NO: 348
  SITE 1: note = N-Terminal Acetyl

SEQ ID NO: 366 - protein, 30 aa, synthetic construct; identical to SEQ ID NO: 293
  SITE 1: note = N-Terminal Acetyl

SEQ ID NO: 367 - protein, 30 aa, synthetic construct; identical to SEQ ID NO: 350
  SITE 1: note = N-Terminal Acetyl

SEQ ID NO: 368 - protein, 15 aa, synthetic construct
  SITE 1: note = N-Terminal Acetyl
PAICQRAEIEACILL

SEQ ID NO: 369 - protein, 15 aa, synthetic construct
  SITE 1: note = N-Terminal Acetyl
PAIAQRAEIEAAILL

SEQ ID NO: 370 - protein, 27 aa, synthetic construct
  SITE 1: note = N-Terminal Acetyl
PAICQRATATLGTNTSGTTEIEACILL

SEQ ID NO: 371 - protein, 23 aa, synthetic construct
  SITE 1: note = N-Terminal Acetyl
PAICQRATATLSGTTEIEACILL

SEQ ID NO: 372 - protein, 19 aa, synthetic construct
  SITE 1: note = N-Terminal Acetyl
PAICQRATATTEIEACILL

SEQ ID NO: 373 - protein, 28 aa, synthetic construct
  SITE 1: note = N-Terminal Acetyl
ICQRATATLGTVGSNTSGTTEIEACILL

SEQ ID NO: 374 - protein, 27 aa, synthetic construct
  SITE 1: note = N-Terminal Acetyl
CQRATATLGTVGSNTSGTTEIEACILL

SEQ ID NO: 375 - protein, 29 aa, synthetic construct
  SITE 1: note = N-Terminal Acetyl
PAICQRATATLGTVGSNTSGTTEIEACIL

SEQ ID NO: 376 - protein, 28 aa, synthetic construct
  SITE 1: note = N-Terminal Acetyl
PAICQRATATLGTVGSNTSGTTEIEACI

SEQ ID NO: 377 - protein, 27 aa, synthetic construct
  SITE 1: note = N-Terminal Acetyl
PAICQRATATLGTVGSNTSGTTEIEAC

SEQ ID NO: 378 - protein, 15 aa, synthetic construct
IVVTAPADLWDWIRA

SEQ ID NO: 379 - protein, 15 aa, synthetic construct
ITVTMPADLWDWIRA

SEQ ID NO: 380 - protein, 15 aa, synthetic construct
IVVTIPADLWDWIRA

SEQ ID NO: 381 - protein, 15 aa, synthetic construct
IVVTLPADLWDWIRA

SEQ ID NO: 382 - protein, 15 aa, synthetic construct
IVVTVPADLWDWIRA

SEQ ID NO: 383 - protein, 15 aa, synthetic construct
IVVTMPADLWDWIMA

SEQ ID NO: 384 - protein, 15 aa, synthetic construct
IVVTMPADLWDWINA

SEQ ID NO: 385 - protein, 15 aa, synthetic construct
IVVTMPADLWDWIQA

SEQ ID NO: 386 - protein, 15 aa, synthetic construct
IHVTIPADLWDWINK

SEQ ID NO: 387 - protein, 14 aa, synthetic construct
IHVTIPADLWDWIN

SEQ ID NO: 388 - protein, 36 aa, synthetic construct
  SITE 4: note = cyclohexyl-L-alanine
  SITE 14: note = 4-fluoro-L-phenylalanine
  SITE 29: note = gamma-carboxyglutamic acid
QSDXDCIHRLLEAXLDPNLTEEQRWEKIXKINDECE

SEQ ID NO: 389 - protein, 37 aa, synthetic construct
  SITE 4: note = cyclohexyl-L-alanine
  SITE 14: note = 4-fluoro-L-phenylalanine
  SITE 29: note = gamma-carboxyglutamic acid
  SITE 31: note = PEG30L
QSDXDCIHRLLEAXLDPNLTEEQRWERIXKXINDECE

SEQ ID NO: 390 - protein, 37 aa, synthetic construct; identical to SEQ ID NO: 389
  SITE 4: note = cyclohexyl-L-alanine
  SITE 14: note = 4-fluoro-L-phenylalanine
  SITE 29: note = gamma-carboxyglutamic acid
  SITE 31: note = PEG20Br

SEQ ID NO: 391 - protein, 37 aa, synthetic construct; identical to SEQ ID NO: 389
  SITE 4: note = cyclohexyl-L-alanine
  SITE 14: note = 4-fluoro-L-phenylalanine
  SITE 29: note = gamma-carboxyglutamic acid
  SITE 31: note = PEG40Br

SEQ ID NO: 392 - protein, 35 aa, synthetic construct
  SITE 3: note = homoleucine
FDXDCIHRLLEAFLDPNLTEQQRWEKIDKINDECE

SEQ ID NO: 393 - protein, 36 aa, synthetic construct
  SITE 4: note = cyclohexyl-L-alanine
  SITE 14: note = 4-fluoro-L-phenylalanine
  SITE 29: note = gamma-carboxyglutamic acid
QSDXDCIHRLLEAXLDPNLTEEQRWERIXKINDECE

SEQ ID NO: 394 - protein, 38 aa, synthetic construct
  SITE 6: note = cyclohexyl-L-alanine
  SITE 19: note = N-epsilon-acetyl-L-Lys
SWQSDXDCIHRLLEAFLDXNLTEEQRWERIDKINDECE

SEQ ID NO: 395 - protein, 38 aa, synthetic construct
  SITE 6: note = cyclohexyl-L-alanine
  MOD_RES 19: note = 40 kDa branched PEG-Lys
SWQSDXDCIHRLLEAFLDKNLTEEQRWERIDKINDECE

SEQ ID NO: 396 - protein, 29 aa, synthetic construct
  VARIANT 3: note = K, Q, R, S, W and Y
  VARIANT 4: note = selected from A, D, E, F, H, I, K, L, M, N, Q, R, S, T, V and Y
  VARIANT 7: note = selected from F, H, I, K, L, M, N, R, S, T, V, W and Y
  VARIANT 11: note = selected from A, I, K, L, M, N, R, S, T and V
  VARIANT 16: note = selected from N and T
  VARIANT 17: note = selected from A, I, T and V
  VARIANT 20: note = selected from I, L, M, R, T and V
  VARIANT 21: note = selected from A, S, T and V
  VARIANT 25: note = selected from I, M, Q, S, T, V and W
  VARIANT 26: note = selected from K and S
  VARIANT 28: note = selected from F, L, M and Y
  VARIANT 29: note = selected from D and R
  VARIANT 18: note = selected from D, E, G, H, K, N, Q, R, S and T
EEXXAWXEIHXLPNLXXXQXXAFIXXLXX

SEQ ID NO: 397 - protein, 29 aa, synthetic construct
  VARIANT 3: note = selected from A, F, H, K, Q, R, S, W and Y
  VARIANT 4: note = selected from A, D, E, F, H, I, K, L, M, N, Q, R, S, T, V and Y
  VARIANT 7: note = selected from F, H, I, K, L, M, N, R, S, T, V, W and Y
  VARIANT 11: note = selected from A, I, K, L, M, N, R, S, T and V
  VARIANT 16: note = selected from N and T
  VARIANT 17: note = selected from A, I, T and V
  VARIANT 18: note = selected from D, E, G, H, K, N, Q, R, S and T
  VARIANT 20: note = selected from I, L, M, R, T and V
  VARIANT 21: note = selected from A, S, T and V
  VARIANT 25: note = selected from I, M, Q, S, T, V and W
  VARIANT 26: note = selected from K and S
  VARIANT 28: note = selected from F, L, M and Y
  VARIANT 29: note = selected from D and R
EEXXAWXEIHXLPNLXXXQXXAFIXXLXX

SEQ ID NO: 398 - protein, 49 aa, synthetic construct
YYALSDAKEEEPRYKALRGENQDLREKERKYQDKIKKLEEKEKNLEKKS

SEQ ID NO: 399 - protein, 17 aa, synthetic construct
DEARRCYNDPKCSDFVQ

SEQ ID NO: 400 - DNA, 15 bp, synthetic construct
  source 1..15: mol_type = other DNA
ggttggtgtggttgg

SEQ ID NO: 401 - protein, 7 aa, synthetic construct
SVWIWYE

SEQ ID NO: 402 - protein, 10 aa, synthetic construct
DVWIINKKLK

SEQ ID NO: 403 - protein, 19 aa, synthetic construct
MLRTKDLIWTLFFLGTAVS

SEQ ID NO: 404 - protein, 25 aa, synthetic construct
MLRTKDLIWTLFFLGTAVSKKRPKP

SEQ ID NO: 405 - protein, 25 aa, synthetic construct
MLRTKDLIWTLFFLGTAVSKKLVFF

SEQ ID NO: 406 - protein, 7 aa, synthetic construct
STPTRYS

SEQ ID NO: 407 - protein, 9 aa, synthetic construct
CALWHWWHC

SEQ ID NO: 408 - protein, 9 aa, synthetic construct
  VARIANT 2: note = Threonine or Serine
CXWLHWWAC

SEQ ID NO: 409 - protein, 25 aa, synthetic construct
  SITE 1..25: note = D-amino acids
DDDEKQLKERWYKRWLEYLDEFKKN

SEQ ID NO: 410 - protein, 25 aa, synthetic construct
  SITE 1..25: note = D-amino acids
TEEEKQLKEWWYKHWQEYLEEFKKN

SEQ ID NO: 411 - protein, 20 aa, synthetic construct
GACPPCLWQVLCGGSGSGSG

SEQ ID NO: 412 - protein, 12 aa, synthetic construct
HIHDDLLRYYGW

SEQ ID NO: 413 - skipped

SEQ ID NO: 414 - protein, 7 aa, synthetic construct
KRWSRYF

SEQ ID NO: 415 - protein, 12 aa, synthetic construct
HIHDDLLRYYGW

SEQ ID NO: 416 - protein, 9 aa, synthetic construct
YCWSQYLCY

SEQ ID NO: 417 - protein, 15 aa, synthetic construct
DFLPHYKNTSLGHRP

SEQ ID NO: 418 - protein, 9 aa, synthetic construct
YCLYQSWCY

SEQ ID NO: 419 - protein, 18 aa, synthetic construct
  MOD_RES 1: note = Alanine Side Chain Modified with Linker
  CROSSLNK 1..18: note = Cyclic Crosslink Bond formed between Alanine-1 and Glutamine-18
  DISULFID 7..13: note = Disulfide Bond between Cysteine-7 and Cysteine-13
ADEARRCYNDPKCSDFVQ

SEQ ID NO: 420 - protein, 18 aa, synthetic construct
ADEARRCYNDPKCSDFVQ

SEQ ID NO: 421 - protein, 10 aa, synthetic construct
  SITE 1: note = D-Aspartate
  SITE 2: note = D-Cysteine
  SITE 3: note = D-Cysteine
  SITE 4: note = D-Lysine
  SITE 5: note = D-Proline
  SITE 6: note = D-Aspartate
  SITE 7: note = D-Asparagine
  SITE 8: note = D-Tyrosine
  SITE 9: note = D-Cysteine
  SITE 10: note = D-Arginine
DCCKPDNYCR

SEQ ID NO: 422 - protein, 5 aa, Synthetic construct
SYAMS

SEQ ID NO: 423 - protein, 5 aa, synthetic construct
DYSMS

SEQ ID NO: 424 - protein, 5 aa, synthetic construct
DYSAS

SEQ ID NO: 425 - protein, 5 aa, synthetic construct
DYSLS

SEQ ID NO: 426 - protein, 16 aa, synthetic construct
AISGSGGSTYYADSVK

SEQ ID NO: 427 - protein, 16 aa, synthetic construct
AISWNGDSTYYAESMK

SEQ ID NO: 428 - protein, 16 aa, synthetic construct
AISWNGDSTYYAESLK

SEQ ID NO: 429 - protein, 16 aa, synthetic construct
AISWNGDSTYYAESAK

SEQ ID NO: 430 - protein, 16 aa, synthetic construct
AISWNGDSTYYAESVK

SEQ ID NO: 431 - protein, 16 aa, synthetic construct
AITWSGDSTYYAESVK

SEQ ID NO: 432 - protein, 16 aa, synthetic construct
AISWSGDSTYYAESLK

SEQ ID NO: 433 - protein, 16 aa, synthetic construct
AISWSGDSTYYAESVK

SEQ ID NO: 434 - protein, 16 aa, synthetic construct
AISWQGDSTYYAESAK

SEQ ID NO: 435 - protein, 16 aa, synthetic construct
AISWNADSTYYAESAK

SEQ ID NO: 436 - skipped

SEQ ID NO: 437 - protein, 16 aa, synthetic construct
SWATPIESLYYYGMDY

SEQ ID NO: 438 - protein, 16 aa, synthetic construct
SWATPIESLYYYGSDY

SEQ ID NO: 439 - protein, 16 aa, synthetic construct
SWATPIESLYYYGTDY

SEQ ID NO: 440 - protein, 11 aa, synthetic construct
GGNNIGSKNVH

SEQ ID NO: 441 - protein, 11 aa, synthetic construct
GGNNLGYKSVH

SEQ ID NO: 442 - protein, 11 aa, synthetic construct
GGNNIGSQTAQ

SEQ ID NO: 443 - protein, 7 aa, synthetic construct
RDSNRPS

SEQ ID NO: 444 - protein, 7 aa, synthetic construct
RDNNRPS

SEQ ID NO: 445 - protein, 7 aa, synthetic construct
ANNRRPS

SEQ ID NO: 446 - protein, 4 aa, synthetic construct
QVVV

SEQ ID NO: 447 - protein, 11 aa, synthetic construct
QVWDGSTQAIV

SEQ ID NO: 448 - protein, 11 aa, synthetic construct
QVWEDSTQAIV

SEQ ID NO: 449 - protein, 11 aa, synthetic construct
QVWDESTQAIV

SEQ ID NO: 450 - protein, 11 aa, synthetic construct
QVWAASTQAIV

SEQ ID NO: 451 - protein, 11 aa, synthetic construct
QVWDDSTQAIV

SEQ ID NO: 452 - protein, 11 aa, synthetic construct
QVWEASTQAIV

SEQ ID NO: 453 - protein, 11 aa, synthetic construct
QVWDASTQAIV

SEQ ID NO: 454 - protein, 11 aa, synthetic construct
QVWEESTQAIV

SEQ ID NO: 455 - protein, 11 aa, synthetic construct
QVWEGSTQAIV

SEQ ID NO: 456 - protein, 5 aa, synthetic construct
DDYMN

SEQ ID NO: 457 - protein, 16 aa, synthetic construct
WIDPENGDTEYASKFQ

SEQ ID NO: 458 - protein, 9 aa, synthetic construct
GYDYFPFVY

SEQ ID NO: 459 - protein, 16 aa, synthetic construct
RSSQNIVHSNGNTYLE

SEQ ID NO: 460 - protein, 7 aa, synthetic construct
KVSNRFS

SEQ ID NO: 461 - protein, 9 aa, synthetic construct
FQGSHVPFT

SEQ ID NO: 462 - protein, 5 aa, synthetic construct
TSGMS

SEQ ID NO: 463 - protein, 16 aa, synthetic construct
WINTYSGEPTYADDFK

SEQ ID NO: 464 - protein, 10 aa, synthetic construct
SRNNYEGFAY

SEQ ID NO: 465 - protein, 16 aa, synthetic construct
KSSQSLLYSSNQKNYL

SEQ ID NO: 466 - protein, 7 aa, synthetic construct
WASTRES

SEQ ID NO: 467 - protein, 9 aa, synthetic construct
QQYYLYPLT

SEQ ID NO: 468 - protein, 23 aa, synthetic construct
  NON_STD 12: note = citrulline
  DISULFID 1..23: note = Cys-Cys
CHHPGIAEFPSXGKSYSYSKQFC

SEQ ID NO: 469 - protein, 27 aa, synthetic construct
  NON_STD 16: note = citrulline
  DISULFID 5..27: note = Cys-Cys
EEEECHHPGIAEFPSXGKSYSYSKQFC

SEQ ID NO: 470 - protein, 23 aa, synthetic construct
  NON_STD 12: note = citrulline
  DISULFID 1..23: note = Cys-Cys
  MOD_RES 1: note = N-hexyl
CHHPGIAEFPSXGKSYSYSKQFC

SEQ ID NO: 471 - protein, 21 aa, synthetic construct
  NON_STD 11: note = citrulline
HHPGIAEFPSXGKSSSYSKQF

SEQ ID NO: 472 - protein, 21 aa, synthetic construct
  NON_STD 11: note = citrulline
HHPGIAEFPSXGKSYSYSKQF

SEQ ID NO: 473 - protein, 21 aa, synthetic construct
  NON_STD 11: note = citrulline
HGPGIAEFPSXGPSYSYSKQF

SEQ ID NO: 474 - protein, 13 aa, synthetic construct
  NON_STD 7: note = citrulline
AEGGGVXGPRVVE

SEQ ID NO: 475 - protein, 13 aa, synthetic construct
  NON_STD 7: note = citrulline
ASSGGVXGPRIVE

SEQ ID NO: 476 - protein, 16 aa, synthetic construct
  NON_STD 7: note = citrulline
  NON_STD 9: note = citrulline
KDLLPSXDXQHLPLIK

SEQ ID NO: 477 - protein, 23 aa, synthetic construct
  NON_STD 8: note = citrulline
  NON_STD 16: note = citrulline
QMRMELEXPGGNEITXGGSTSYG

SEQ ID NO: 478 - protein, 14 aa, synthetic construct
  NON_STD 7: note = citrulline
  NON_STD 8: note = citrulline
NVSPGTXXEYHTEK

SEQ ID NO: 479 - protein, 16 aa, synthetic construct
  NON_STD 3: note = citrulline
  NON_STD 11: note = citrulline
  NON_STD 12: note = citrulline
STXSVSSSSYXXMFGG

SEQ ID NO: 480 - protein, 16 aa, synthetic construct
  NON_STD 5: note = citrulline
  NON_STD 10: note = citrulline
  NON_STD 12: note = citrulline
VYATXSSAVXLXSSVP

SEQ ID NO: 481 - protein, 16 aa, synthetic construct
  NON_STD 3: note = citrulline
  NON_STD 8: note = citrulline
AXTKQTAXKSTGGKAP

SEQ ID NO: 482 - protein, 16 aa, synthetic construct
  NON_STD 3: note = citrulline
AAXKSAPSTGGVKKPH

SEQ ID NO: 483 - protein, 17 aa, synthetic construct
  NON_STD 2: note = citrulline
  NON_STD 9: note = citrulline
YXPGTVALXEIKKYQKS

SEQ ID NO: 484 - protein, 19 aa, synthetic construct
  NON_STD 3: note = citrulline
  NON_STD 9: note = citrulline
  NON_STD 12: note = citrulline
LIXKLPFQXLVXEIAQDFK

SEQ ID NO: 485 - protein, 16 aa, synthetic construct
  NON_STD 8: note = citrulline
LCAIHAKXVTIMPKDI

SEQ ID NO: 486 - protein, 11 aa, synthetic construct
  NON_STD 2: note = citrulline
  NON_STD 7: note = citrulline
AXGLTGXPGDA

SEQ ID NO: 487 - protein, 19 aa, synthetic construct
  NON_STD 7: note = citrulline
  NON_STD 9: note = citrulline
  NON_STD 11: note = citrulline
SHQESTXGXSXGRSGRSGS

SEQ ID NO: 488 - protein, 5 aa, synthetic construct
  NON_STD 2: note = citrulline
TXGRS

SEQ ID NO: 489 - skipped

SEQ ID NO: 490 - protein, 5 aa, synthetic construct
  NON_STD 4: note = citrulline
TRGXS

SEQ ID NO: 491 - protein, 21 aa, synthetic construct
HHPGIAEFPSRGKSSSYSKQF

SEQ ID NO: 492 - protein, 21 aa, synthetic construct
  NON_STD 11: note = citrulline
HHPGIAEFPSXGKSSSYSKQF

SEQ ID NO: 493 - protein, 21 aa, synthetic construct
SKQFTSSTSYNRGDSTFESKS

SEQ ID NO: 494 - protein, 21 aa, synthetic construct
  NON_STD 12: note = citrulline
SKQFTSSTSYNXGDSTFESKS

SEQ ID NO: 495 - protein, 20 aa, synthetic construct
APPPISGGGYRARPAKAAAT

SEQ ID NO: 496 - protein, 20 aa, synthetic construct
  NON_STD 11: note = citrulline
APPPISGGGYXARPAKAAAT

SEQ ID NO: 497 - protein, 20 aa, synthetic construct
  NON_STD 13: note = citrulline
APPPISGGGYRAXPAKAAAT

SEQ ID NO: 498 - protein, 22 aa, synthetic construct
  NON_STD 12: note = citrulline
  REGION 1: note = circular
CHHPGIAEFPSXGKSSSYSKQF

SEQ ID NO: 499 - protein, 15 aa, synthetic construct
  NON_STD 8: note = citrulline
GIAEFPSXGKSSSYS

SEQ ID NO: 500 - protein, 11 aa, synthetic construct
  NON_STD 6: note = citrulline
AEFPSXGKSSS

SEQ ID NO: 501 - protein, 560 aa, synthetic construct
AWITAPVALREGEDLSKKNPIAKIHSDLAEERGLKITYKYTGKGITEPPFGIFVFNKDTGELNVTSILDREETPFFLLTGYALDARGNNVEKPLELRIKVLDINDNEPVFTQDVFVGSVEELSAAHTLVMKINATDADEPNTLNSKISYRIVSLEPAYPPVFYLNKDTGEIYTTSVTLDREEHSSYTLTVEARDGNGEVTDKPVKQAQVQIRILDVNDNIPVVENKVLEGMVEENQVNVEVTRIKVFDADEIGSDNWLANFTFASGNEGGYFHIETDAQTNEGIVTLIKEVDYEEMKNLDFSVIVANKAAFHKSIRSKYKPTPIPIKVKVKNVKEGIHFKSSVISIYVSESMDRSSKGQIIGNFQAFDEDTGLPAHARYVKLEDRDNWISVDSVTSEIKLAKLPDFESRYVQNGTYTVKIVAISEDYPRKTITGTVLINVEDINDNCPTLIEPVQTICHDAEYVNVTAEDLDGHPNSGPFSFSVIDKPPGMAEKWKIARQESTSVLLQQSEKKLGRSEIQFLISDNQGFSCPEKQVLTLTVCECLHGSGCREAQHDSYVG

SEQ ID NO: 502 - protein, 12 aa, synthetic construct
  SITE 12: note = C-terminal amino acid is linked to another sequence
HIHFFLLRYYGW

SEQ ID NO: 503 - protein, 13 aa, synthetic construct
  SITE 13: note = C-terminal amino acid is linked to another sequence
  SITE 13: note = side chain of lysine is linked via an amide linkage to another sequence
HIHFFLLRYYGWK

SEQ ID NO: 504 - protein, 14 aa, synthetic construct
  SITE 13: note = side chain of lysine is linked via an amide linkage to another sequence
  SITE 14: note = side chain of lysine is linked via an amide linkage to another sequence
  SITE 14: note = C-terminal amide
HIHFFLLRYYGWKK